(19) United States
(12) Patent Application Publication
(10) Pub. No.: US 2018/0057505 A1
(43) Pub. Date: Mar. 1, 2018

(54) BICYCLIC HETEROARYL COMPOUNDS USEFUL AS INHIBITORS OF THE PAR-2 SIGNALING PATHWAY

(71) Applicant: VERTEX PHARMACEUTICALS INCORPORATED, Boston, MA (US)

(72) Inventors: Camil Elie SAYEGH, Dorval (CA); Claudio STURINO, Ile Bizard (CA); Pierre-Andre FOURNIER, Vaudreuil-Dorion (CA); Jean-Eric LACOSTE, Laval (CA); Evelyne DIETRICH, Laval (CA); Julien MARTEL, Montreal (CA); Frederic VALLEE, Montreal (CA); Janek SZYCHOWSKI, Montreal (CA); Miguel ST-ONGE, Vaudreuil-Dorion (CA); Monica BUBENIK, Mascouche (CA); Stephanie LESSARD, St-Jean-sur-Richelieu (CA); Yeeman RAMTOHUL, Pierrefonds (CA)

(21) Appl. No.: 15/559,761

(22) PCT Filed: Mar. 19, 2016
(86) PCT No.: PCT/US2016/023324

§ 371 Date: Sep. 19, 2017

Related U.S. Application Data
(60) Provisional application No. 62/135,919, filed on Mar. 20, 2015.

(51) Int. Cl. C07D 491/052; C07D 498/04; C07D 513/04; C07H 15/26; C07F 9/6561

(52) U.S. Cl. CPC C07D 491/052; C07H 15/26; C07F 9/6561; C07D 513/04; C07D 498/04

(57) ABSTRACT

The compounds of formula I, wherein the variables are as defined herein, and pharmaceutically acceptable salts thereof are useful as inhibitors of the PAR-2 signaling pathway. The compounds of formula I and pharmaceutically acceptable compositions comprising such compounds can be employed for treating various diseases, disorders, and conditions.

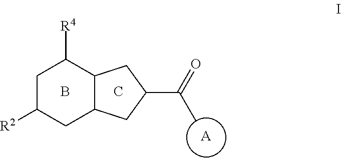

### RELATED APPLICATIONS

The present application claims priority to United States Provisional Application Nos. 62/135,919 filed on Mar. 20, 2015. The entire contents of this application are incorporated herein by reference.

### BACKGROUND OF THE INVENTION

Protease-Activated Receptors (PARs) are a family of G-protein coupled receptors (GPCRs) comprising PAR-1, 2, 3, and 4. PARs are typically activated when enzymes (such as thrombin or trypsin) proteolytically cleave a portion of their N-terminal sequence. This cleavage exposes a region of the N-terminal extracellular domain (called the “tethered ligand”) which is believed to bind to residues contained within the second extracellular loop of the PAR receptors, resulting in the stabilization of an active conformation. Short synthetic peptides mimicking the tethered ligand sequence have been successfully used to activate all of the PAR receptors, except PAR-3.

PAR-2 is activated by several host and pathogen-derived serine proteases, including trypsin, mast cell tryptase, certain tissue kallikreins, and members of the coagulation cascade TF-FVIIa and FVa-FXa. Synthetic ligands such as SLIGKV-NH₂ can selectively activate human PAR-2, although modified PAR-2 synthetic agonists such as 2-fluoryl-LIGRLO-NH2 have been reported to be more potent activators of this receptor.

PAR-2 has been shown to be an important receptor in mediating inflammation, pain and itch. For example, PAR-2 activation results in inflammatory cytokine and chemokine release from keratinocytes, endothelial cells and from human epithelial cell lines such as A549. Moreover, the administration of PAR-2 activating proteases and synthetic agonists in vivo induce inflammatory responses. In particular, several studies have shown that intraplantar administration of PAR-2 agonists in rodents results in an edema response that is dependent in part on neuronal PAR-2 activation.

Similar studies have implicated PAR-2 as a mediator of neurogenic inflammation, nociception and in transmission of pain. This is mediated in part by the activation of PAR-2 dependent signaling pathways in dorsal root ganglia, the release of neuropeptides from C-fibers in peripheral tissues and spinal cord and the potentiation of transient receptor potential vaniloid 1 and 4 receptors in sensory neurons.

Several studies have demonstrated a role for PAR-2 activation in pruritus. Both direct activation of PAR-2 on nerve endings and indirect effects of PAR-2 on resident cells including keratinocytes are thought to contribute to itch.

Further, both in vitro and in vivo studies have demonstrated a role for PAR-2 activation in tissue remodeling. First, activation of PAR-2 can promote fibroblast and myofibroblast proliferation, and the secretion of growth factors such as CTGF and extracellular matrix components including collagen. In addition. PAR-2 activation was shown to be implicated in cellular migration and activation of this pathway has recently been shown to promote tumor growth and metastasis.

Numerous studies relying on the use of PAR-2 deficient mice, blocking PAR-2 antibodies or PAR-2 antagonists such as GB88 revealed an important role for PAR-2 activation in the pathophysiology of a variety of diseases including asthma, chronic pain, rheumatoid arthritis, periodontitis, inflammatory bowel diseases, irritable bowel syndrome, skin diseases, cancer, fibrotic diseases and neurological disease (reviewed in Yau et al, Journal of Medicinal Chemistry, July 2013). Other studies have shown that diet-induced obesity, adipose inflammation, and metabolic dysfunction correlating with PAR-2 expression are attenuated by PAR-2 antagonism.

For all of these reasons, there is a need for the development of potent and selective inhibitors of the PAR-2 signaling pathway as novel therapeutic agents.

### SUMMARY OF THE INVENTION

The present invention relates to compounds useful as inhibitors of the PAR-2 signaling pathway. The invention also relates to pharmaceutically acceptable compositions comprising the compounds of this invention; methods of treating of various diseases, disorders, and conditions using the compounds of this invention; processes for preparing the compounds of this invention; intermediates for the preparation of the compounds of this invention; and methods of using the compounds in in vitro applications.

One aspect of the invention provides a compound of Formula I:

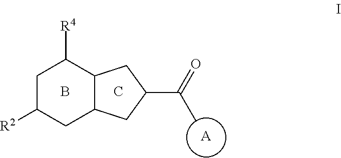

or a pharmaceutically acceptable salt thereof, wherein
Ring B-C is selected from the group consisting of

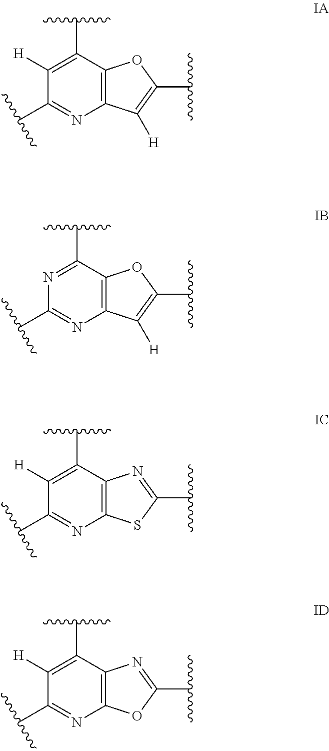 [SMILES: *c1cc(*)c2oc(*)cc2n1 ; *c1nc(*)c2oc(*)cc2n1 ; *c1cc(*)c2nc(*)sc2n1 ; *c1cc(*)c2nc(*)oc2n1]

In one embodiment,

### Ring A is

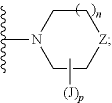

wherein
n is 1 or 2:
Z is —O—, —CH₂—, —NX—, or —CRX³:
X is R⁵, —C(O)R⁵, or —S(O)₂R:
X³ is —(CR₂)ᵣC(O)OR⁶, —(CR₂)ᵣ—N(R)R⁶, —(CR₂)ᵣC(O)N(R)R⁶ or —(CR₂)—C(O)N(R)S(O)₂R⁶; or optionally X³ and J, together with the atoms to which they are bound, form a 5-6 membered aromatic monocyclic ring having 0-2 heteroatoms selected from oxygen, nitrogen, or sulfur; wherein said 5-6 membered ring forms a fused ring together with Ring A, and is optionally substituted with 1-4 occurrences of substituents selected from oxo, halogen, —CN, —OH, —O(C₁₋₄ alkyl), —O(haloC₁₋₄ alkyl), C₁₋₄ alkyl, or haloC₁₋₄ alkyl;
J is CN, oxo, a C₁₋₆aliphatic group wherein up to three carbon units of said C₁₋₆-aliphatic can each be optionally and independently replaced with —O—, —NR—, —S—, or —C(O)—; or a 3-7 membered, saturated, partially unsaturated or aromatic, monocyclic ring having 0-4 heteroatoms selected from oxygen, nitrogen, or sulfur: wherein said J is optionally and independently substituted with 1-4 occurrences of halogen, —CN, or C₁₋₄alkyl, wherein up to one methylene unit of said C₁₋₄alkyl is optionally replaced with —O—, —NR—, or —S—, and wherein said C₁₋₄alkyl is optionally substituted with 1-4 occurrences of halogen or —CN;
or two J groups on the same or different atom(s), together with the atom(s) to which they are bound, form a 3-6 membered, saturated monocyclic ring having 0-2 heteroatoms selected from oxygen, nitrogen, or sulfur: wherein said 3-6 membered ring is optionally substituted with one occurrence of oxo;
p is 0, 1, 2, 3, or 4;
each r is independently 0, 1 or 2;
each of R⁵ and R⁶ is independently —(V)ₐ—Y; wherein
   V is C₁₋₆aliphatic wherein up to three carbon units of said C₁₋₆aliphatic can each be optionally and independently replaced with —O—, —NR—, —S—, —C(O)— or —S(O)₂—; wherein V is optionally substituted with 1-4 occurrences of J^{V};
   J^{V} is halogen, CN, haloC₁₋₄alkyl, or C₁₋₄alkyl, wherein up to one methylene unit of each of said C₁₋₄alkyl and haloC₁₋₄alkyl is optionally replaced with —O—, —NR—, —S—, or —C(O)—;
   Y is H, —CN, a 3-7 membered, saturated, partially unsaturated or aromatic, monocyclic ring having 0-4 heteroatoms selected from oxygen, nitrogen, or sulfur; or a 6-10 membered, saturated, partially unsaturated or aromatic, bicyclic ring having 0-6 heteroatoms selected from oxygen, nitrogen, or sulfur: wherein Y is optionally substituted with 1-4 occurrences of J^{Y};
   J^{Y} is H: oxo: halogen; CN: phenyl; 5-6-membered heteroaryl having 1-4 heteteroatoms selected from oxygen, nitrogen, or sulfur: or C₁₋₆aliphatic, wherein up to three carbon units of said C₁₋₆aliphatic can each be optionally and independently replaced with —O—, —NR—, —S—, —C(O)—, or —S(O)₂—; and wherein each of the phenyl, 5-6 membered heteroaryl and the C₁₋₆aliphatic is optionally and independently substituted with 1-4 occurrences of substituents selected from the group consisting of halogen, —CN, —OH, —OCH₃, —C(O)OH, —OP(O)(OH)₂, —P(O)(R)OH), or

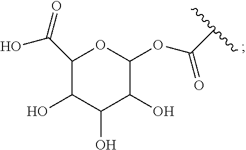 [SMILES: *C(=O)OC1OC(C(=O)O)C(O)C(O)C1O]

and
each R is independently H or C₁₋₄alkyl;
R² is —(V²)_{b}—Y²; wherein
   V² is a C₁₋₄aliphatic;
   Y² is halogen; C₁₋₆aliphatic; a 3-7 membered, saturated, partially unsaturated or aromatic, monocyclic ring having 0-4 heteroatoms selected from oxygen, nitrogen, or sulfur; or a 6-10 membered, saturated, partially unsaturated or aromatic, bicyclic ring having 0-6 heteroatoms selected from oxygen, nitrogen, or sulfur; wherein Y² is optionally substituted with 1-4 occurrences of J^{Y}; and
a and b are each independently 0 or 1;
R⁴ is halogen; CN; C₁₋₆aliphatic wherein up to three carbon units of said C₁₋₆aliphatic can each be optionally and independently replaced with —O—, —NR—, —S— or —C(O)—; a 3-7 membered saturated, partially saturated, or aromatic monocyclic ring having 0-4 heteroatoms selected from oxygen, nitrogen, or sulfur; or a 6-10 membered, saturated, partially unsaturated or aromatic, bicyclic ring having 0-6 heteroatoms selected from oxygen, nitrogen, or sulfur; wherein said R⁴ is optionally and independently substituted with 1-4 occurrences of oxo, halogen, CN, or C₁₋₆aliphatic wherein up to three carbon units of said C₁₋₆aliphatic can each be optionally and independently replaced with —O—, —NR—, —S— or —C(O)—.

In another embodiment,

### Ring A is

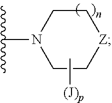

wherein
n is 1 or 2;
Z is —O—, —CH₂—, or —NX—;
X is R⁵, —C(O)R⁵, or —S(O)₂R⁵:
J is —CN, oxo, a C₁₋₆aliphatic group wherein up to three carbon units of said C₁₋₆aliphatic can each be optionally and independently replaced with —O—, —NR—, —S— or —C(O)—; or a 3-7 membered saturated, partially unsaturated, or aromatic monocyclic ring having 0-4 heteroatoms selected from oxygen, nitrogen, or sulfur; wherein said J is optionally and independently substituted with 1-4 occurrences of halogen, —CN, or C₁₋₄alkyl, wherein up to one methylene unit of said C₁₋₄alkyl is optionally replaced with —O—, —NR—, or —S—, and wherein said C₁₋₄alkyl is optionally substituted with 1-4 occurrences of halogen or CN;
or two J groups on the same or different atom(s), together with the atom(s) to which they are bound, form a 3-6 membered saturated monocyclic ring having 0-2 heteroatoms selected from oxygen, nitrogen, or sulfur: wherein said 3-6 membered ring is optionally substituted with one occurrence of oxo;
p is 0-4;
each R⁵ is independently —(V)ₐ—Y; wherein
   V is C₁₋₆aliphatic wherein up to three carbon units of said C₁₋₆aliphatic can each be optionally and independently replaced with —O—, —NR—, —S—, or —C(O)—; wherein V is optionally substituted with 1-4 occurrences of J^{V};
   J^{V} is halogen, —CN, or C₁₋₄alkyl, wherein up to one methylene unit of said C₁₋₄alkyl is optionally replaced with —O—, —NR—, or —S—;
   Y is H, a 3-7 membered saturated, partially unsaturated, or aromatic monocyclic ring having 0-4 heteroatoms selected from oxygen, nitrogen, or sulfur; or a 6-10 membered saturated, partially unsaturated, or aromatic bicyclic ring having 0-6 heteroatoms selected from oxygen, nitrogen, or sulfur: wherein Y is optionally substituted with 1-4 occurrences of J^{Y};
   J^{Y} is H, oxo, halogen, CN, phenyl, or C₁₋₆aliphatic, wherein up to three carbon units of said C₁₋₆aliphatic can each be optionally and independently replaced with —O—, —NR—, —S—, or —C(O)—; and wherein each of the phenyl and the C₁₋₆aliphatic is optionally and independently substituted with 1-4 occurrences of halogen or CN; and
each R is independently H or C₁₋₄alkyl;
R² is —(V²)_{b}—Y²; wherein
   V² is a C₁₋₄aliphatic;
   Y² is halogen; C₁₋₆aliphatic: or a 3-7 membered saturated, partially unsaturated, or aromatic monocyclic ring having 0-4 heteroatoms selected from oxygen, nitrogen, or sulfur; wherein Y² is optionally substituted with 1-4 occurrences of J^{Y}; and
a and b are each independently 0 or 1;
R⁴ is halogen: CN; C₁₋₆aliphatic wherein up to three carbon units of said C₁₋₆aliphatic can each be optionally and independently replaced with —O—, —NR—, —S— or —C(O)—; a 3-7 membered saturated, partially saturated, or aromatic monocyclic ring having 0-4 heteroatoms selected from oxygen, nitrogen, or sulfur; or a 6-10 membered saturated, partially unsaturated, or aromatic bicyclic ring having 0-6 heteroatoms selected from oxygen, nitrogen, or sulfur; wherein said R⁴ is optionally and independently substituted with 1-4 occurrences of oxo, halogen, —CN, or C₁₋₆aliphatic wherein up to three carbon units of said C₁₋₆aliphatic can each be optionally and independently replaced with —O—, —NR—, —S— or —C(O)—.

Another aspect of the invention provides a pharmaceutical composition comprising a compound described herein and a pharmaceutically acceptable carrier, adjuvant, or excipient.

The invention also provides a method for treating a PAR-2 mediated disease in a patient. The method comprises administering to the patient a compound described herein or a pharmaceutically acceptable salt thereof.

The invention also provides a method for treating, preventing, or reducing inflammation or nociception (pain) in a patient comprising administering to the patient a compound described herein or a pharmaceutically acceptable salt thereof.

The invention also provides a method for treating inflammatory bowel disease, Crohn's disease, irritable bowel syndrome, ulcerative colitis, asthma, rheumatoid arthritis, osteoarthritis, fibrosis, gingivitis, atopic dermatitis, psoriasis, systemic lupus erythematosus (SLE), scleroderma, interstitial lung disease, polymyositis, periodontitis, vasculitis, Netherton syndrome, atopic dermatitis, dermatomyositis, uveitis, Alzheimer's disease, Parkinson's disease, multiple sclerosis, inflammatory pain, post-operative incision pain, neuropathic pain, fracture pain, osteroporotic fracture pain, gout joint pain, cancer, diet-induced obesity, adipose inflammation, and metabolic dysfunction correlating with PAR2 expression in a patient comprising administering a compound described herein or a pharmaceutically acceptable salt thereof.

The invention also provides a method of inhibiting proteolytic activation of PAR-2 in a cell comprising administering to a patient or to a biological sample a compound described herein or a pharmaceutically acceptable salt thereof.

The invention also provides a method of inhibiting PAR-2 activity in a cell comprising administering to a patient or to a biological sample a compound of described herein or a pharmaceutically acceptable salt thereof.

The invention also provide use of the compounds of the invention for treating the diseases and conditions disclosed herein. Use of the compounds of the invention in the manufacture of a medicament for treating the diseases and conditions disclosed herein is also included in the invention.

Another aspect of the invention includes a method of preparing a compound represented by Structural Formula (I) or a pharmaceutically acceptable salt thereof, wherein the variables of formula (I) are each and independently as described herein. The method comprises reacting Compound (X-1) with Compound (Y-1) to form a compound of formula (I) or a pharmaceutically acceptable salt thereof:

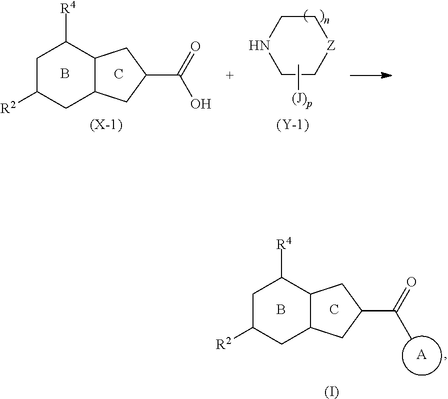

wherein the variables of Compound (X-1) with Compound (Y-1) are each and independently as described for formula (I).

Another aspect of the invention includes a method of preparing a compound represented by Structural Formula (I) or pharmaceutically acceptable salt thereof, wherein Rings B and C, and R² and R⁴ are each and independently as defined herein, and Ring A is

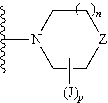

wherein Z is —NX—. The method comprises reacting Compound (X-2) with X-L¹ to form a compound of formula (I) or a pharmaceutically acceptable salt thereof:

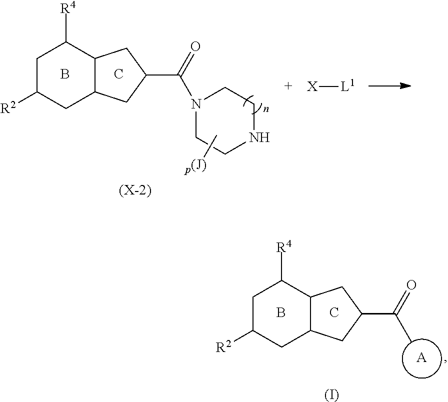

wherein X of X-L¹ is as described for formula (I) and L¹ of X-L¹ is from halo or —OH, and wherein the variables of Compound (X-2) are each and independently as described for Formula (I).

Another aspect of the invention includes a method of preparing a compound of formula (I) or pharmaceutically acceptable salt thereof, wherein the variables of formula (I) are each and independently as described herein. The method comprises reacting Compound (X-3) with R²-L³ to form a compound of formula (I) or a pharmaceutically acceptable salt thereof:

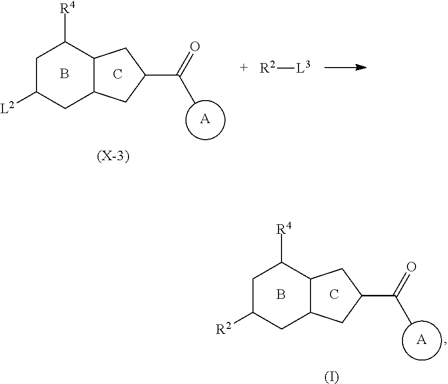

wherein L² of Compound (X-3) is halo, and the remaining variables of Compound (X-3) are each and independently as described for formula (I), and wherein L³ of R²-L³ is —B(OR^{a})₂, wherein R^{a} is —H or two R^{a} together with the atom to which they are attached form a dioxaborolane optionally substituted with C₁₋₂alkyl, and R² of R²-L³ is as described for formula (I).

The compounds of the invention are potent inhibitors of the PAR-2 signaling pathway. These compounds can be used for reduction in inflammation in in vivo models of inflammation.

### DETAILED DESCRIPTION OF THE INVENTION

In one embodiment, the invention is directed to compounds represented by formula (I) and pharmaceutically acceptable salts thereof:

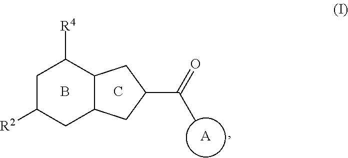

wherein the variables of formula (I) are each and independently as described herein.

In the first set, the variables of formula (I) are as described below:

### Ring A is

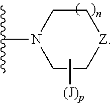

In a specific embodiment, Ring A is

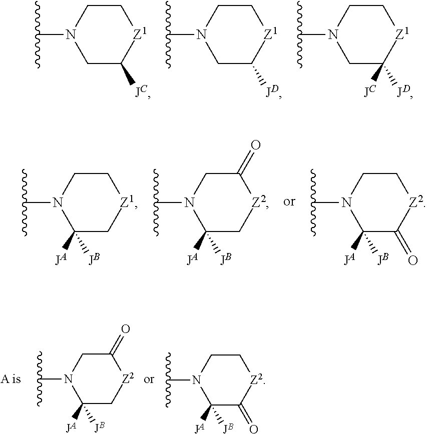

In another specific embodiment, Ring A is

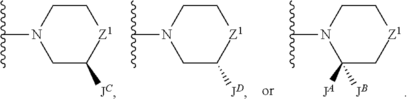

In yet another specific embodiment, Ring A is

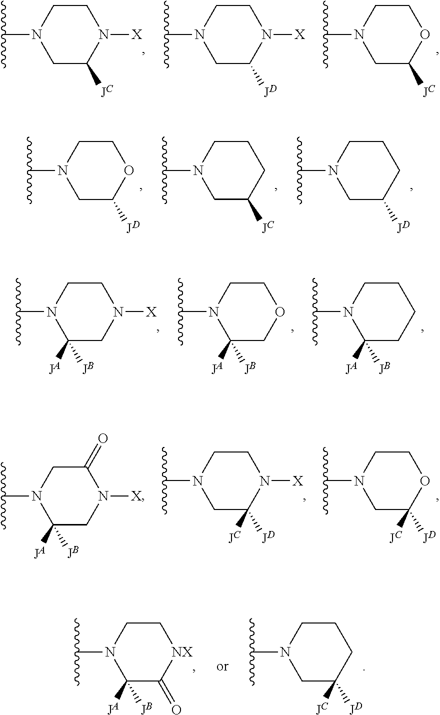

In yet another specific embodiment, Ring A or is

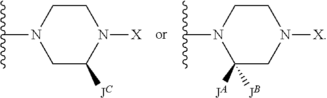

In yet another specific embodiment, Ring A is A is

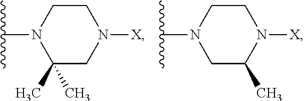

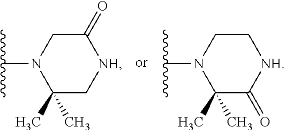

In yet another specific embodiment, Ring A is

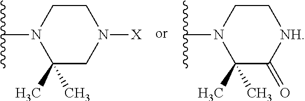

In yet another specific embodiment, Ring A is

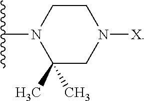

In yet another specific embodiment, Ring A is

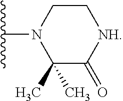

In yet another specific embodiment, Ring A is

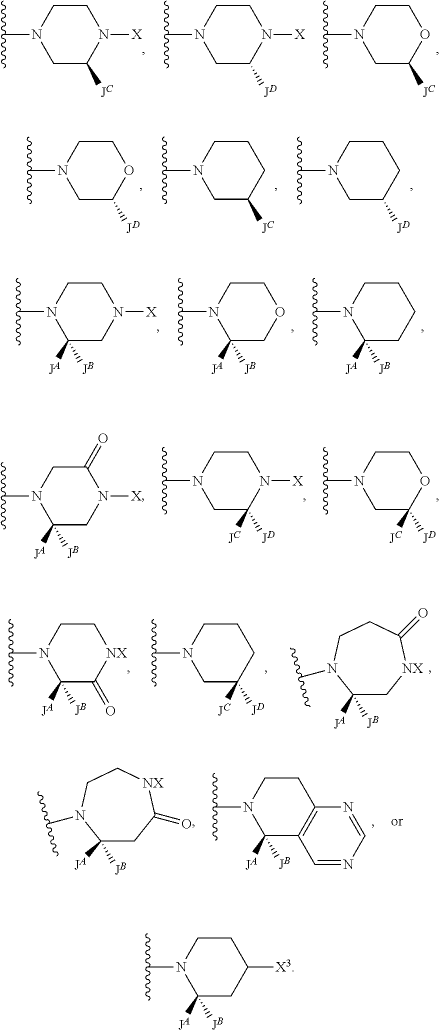

In yet another specific embodiment, Ring A is

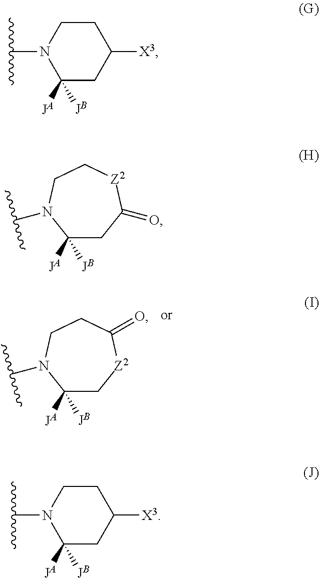

In yet another specific embodiment, Ring A is

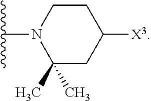

Ring B-C is selected from Ring IA, IB, IC, or ID:

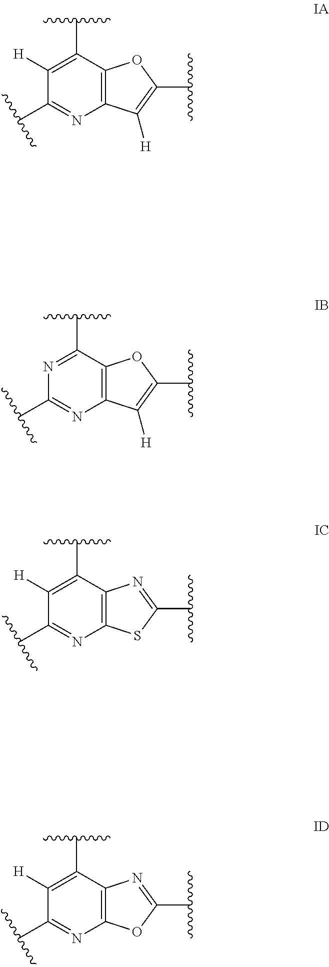 [SMILES: *c1cc(*)c2oc(*)cc2n1 ; *c1nc(*)c2oc(*)cc2n1 ; *c1cc(*)c2nc(*)sc2n1 ; *c1cc(*)c2nc(*)oc2n1]

In a specific embodiment, Ring B-C is Ring IA.

Z is —O—, —CH₂—, —NX—, or —CRX³. Alternatively, Z is —O—, —CH₂—, or —NX—. In one specific embodiment, Z is —NX—. In another specific embodiment, Z is —CRX³.

Z¹ is —O—, —CH₂—, or —NX—, and X is X¹.

Z² is —CH₂— or —NX—, and X is X².

X is R⁵, —C(O)R⁵, or —S(O)₂R⁵. In a specific embodiment, X is —R⁵ or C(O)R⁵. In another specific embodiment, X is C₁₋₆alkyl, —Y, —C(O)—C₁₋₆ alkyl, —S(O)₂—C₁₋₃ alkyl, —C(O)—(CH₂)_{q}—C₁₋₃ alkyl, —C(O)N(R)S(O)₂—C₁₋₃ alkyl, —C(O)—(CH₂)_{q}—N(R)S(O)₂—C₁₋₃ alkyl, —C(O)N(R)S(O)₂—C₁₋₃ alkyl, —C(O)—(CH₂)_{q}—S(O)₂—N(R)—C₁₋₂ alkyl, —C(O)N(R)—C₁₋₃ alkyl, —C(O)—(CH₂)_{q}—S(O)₂—C₁₋₃ alkyl. —C(O)Y; —S(O)₂Y, —C(O)—(CH₂)_{q}—Y, —C(O)N(R)S(O)₂Y, —C(O)—(CH₂)_{q}—N(R)S(O)₂Y, —C(O)N(R)S(O)₂Y, —C(O)—(CH₂)_{q}—S(O)₂—N(R)Y, —C(O)N(R)Y, —C(O)—(CH₂)_{q}—S(O)₂Y, wherein each q is independently 1 or 2; each of said C₁₋₆ alkyl, C₁₋₃ alkyl and C₁₋₂ alkyl of X is optionally and independently substituted with 1-4 occurrences of J^{V}, wherein each J^{V} is independently selected from CN, halogen, C₁₋₄alkyl, haloC₁₋₄ alkyl, —OH, —O(C₁₋₂alkyl), —NH(C₁₋₂alkyl), —N(C₁₋₂alkyl)₂, —S(O)₂(C₁₋₂alkyl), —NH₂, —C(O)OH, —CH₂—C(O)OH, —(CH₂)₂—C(O)OH, —CH₂—C(O)OCH₃, or —C(O)O(C₁₋₂ alkyl); and each Y is independently optionally substituted with 1-4 occurrences of J^{Y}, wherein each J^{Y} is independently selected from oxo, CN, halogen, C₁₋₄alkyl, haloC₁₋₄alkyl, —OH, —O(C₁₋₄alkyl), NH(C₁₋₄alkyl), N(C₁₋₄alkyl)₂, S(O)₂(C₁₋₄alkyl), phenyl, NH₂, tetrazole, (CH₂)—C(O)OH, (CH₂)ₜ—C(O)O(C₁₋₂ alkyl), (CH(C₁₋₂ alkyl))ₜ—C(O)OH, (CH(C₁₋₂ alkyl))ₜ—C(O)O(C₁₋₂ alkyl), (C(C₁₋₂ alkyl)₂)ₜ−C(O)OH, (C(C₁₋₂ alkyl)₂)ₜ—C(O)O(C₁₋₂ alkyl), or —C(O)O—CH₂—O—P(O)(OH)₂, wherein each t is independently 0 or 1.

In yet another specific embodiment, X is selected from the group consisting of:

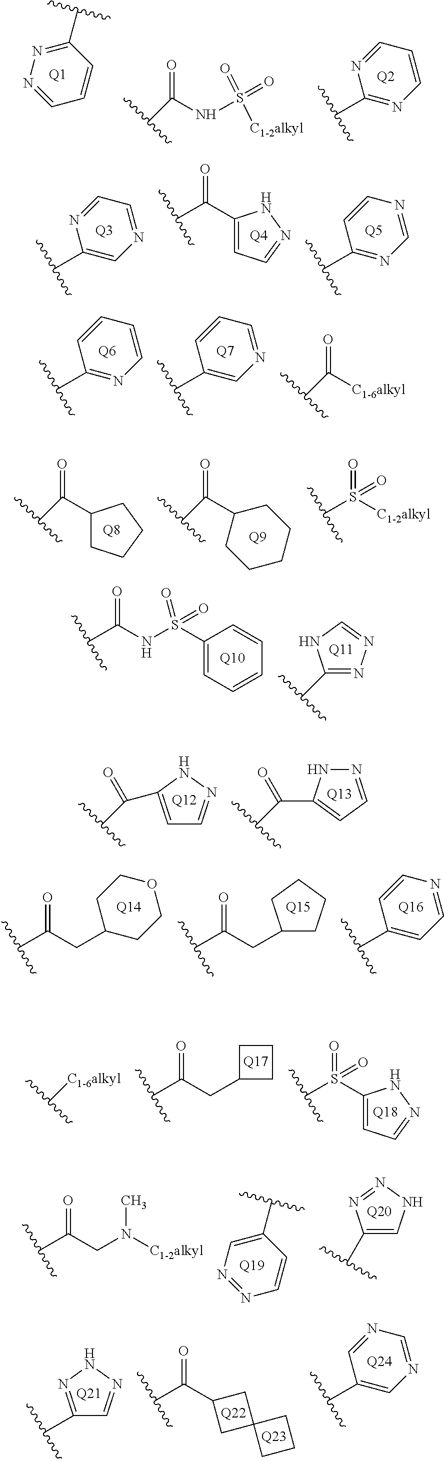

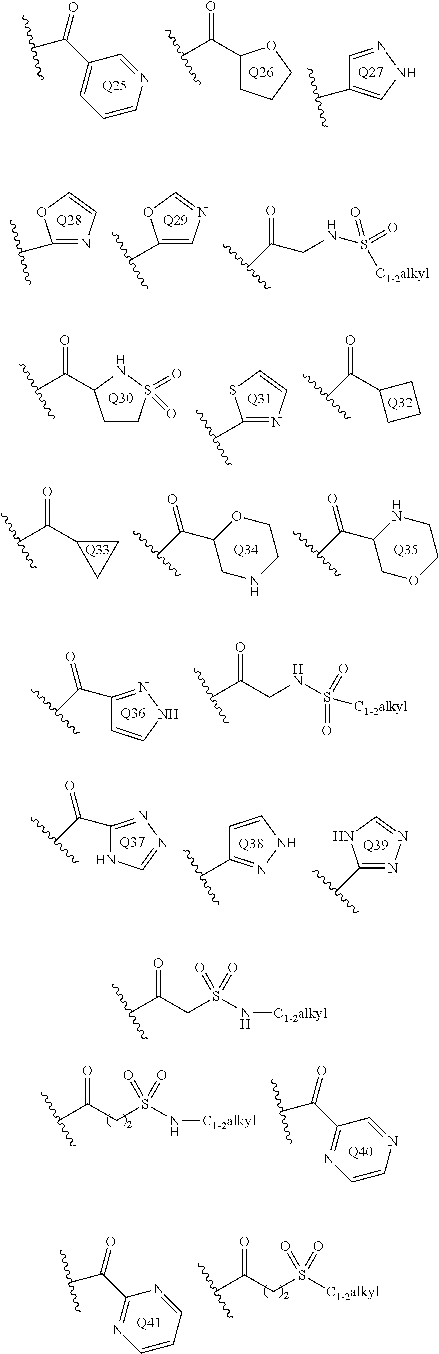

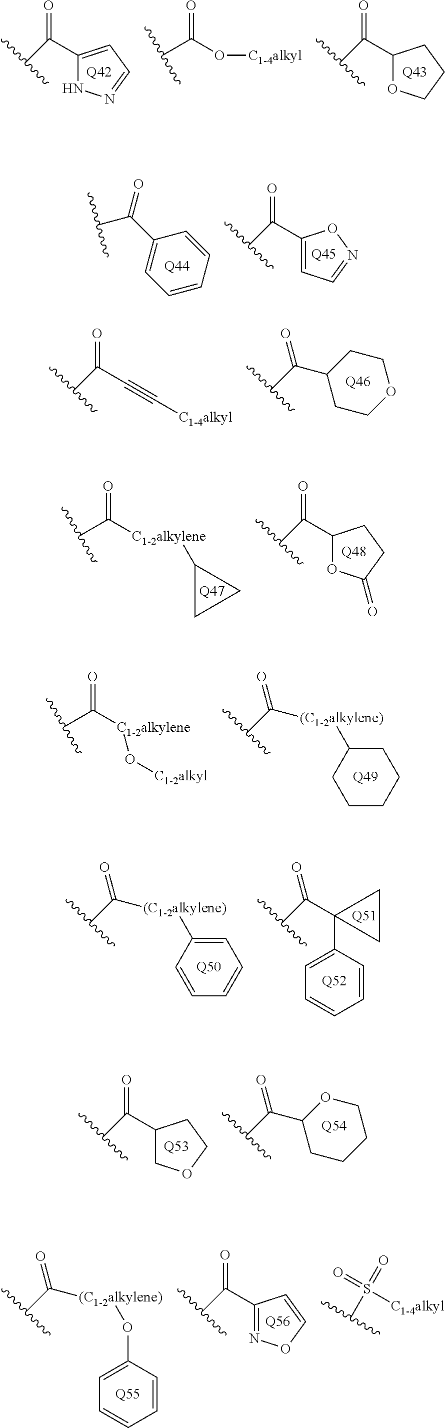

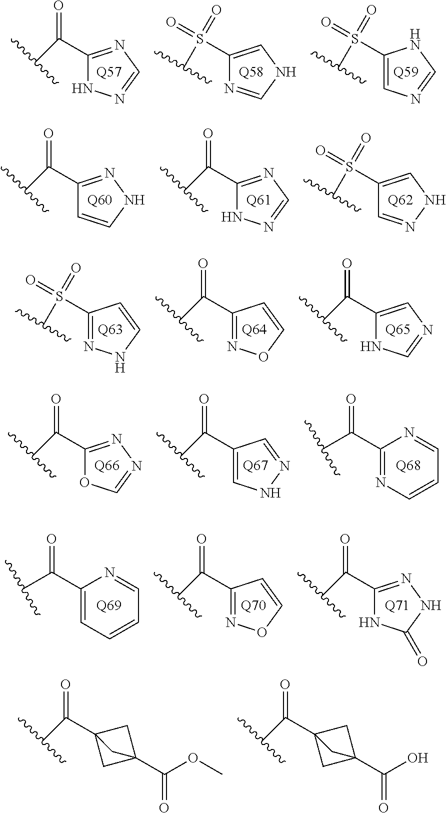

wherein each of Rings Q1-Q71 is optionally and independently substituted with 1-4 occurrences of J^{Y}; and each of said C₁₋₂alkyl, C₁₋₂alkylene, C₁₋₄alkyl, and C₁₋₆alkyl of X is optionally and independently substituted with 1-4 occurrences of J^{V}. In some specific embodiment, J^{Y} is —CN, halo, —CH₃, —CF₃, —OH, —OCH₃, —NH₂, —NHCH₃, N(CH₃)₂, —C(O)OH, or —C(O)O(C₁₋₂ alkyl); and J^{V} is —CN, halo, —CH₃, —CF₃, —OH, —OCH₃, —NH₂, —NHCH₃, N(CH₃)₂, —C(O)OH, or —C(O)O(C₁₋₂ alkyl). In yet another specific embodiment, X is:

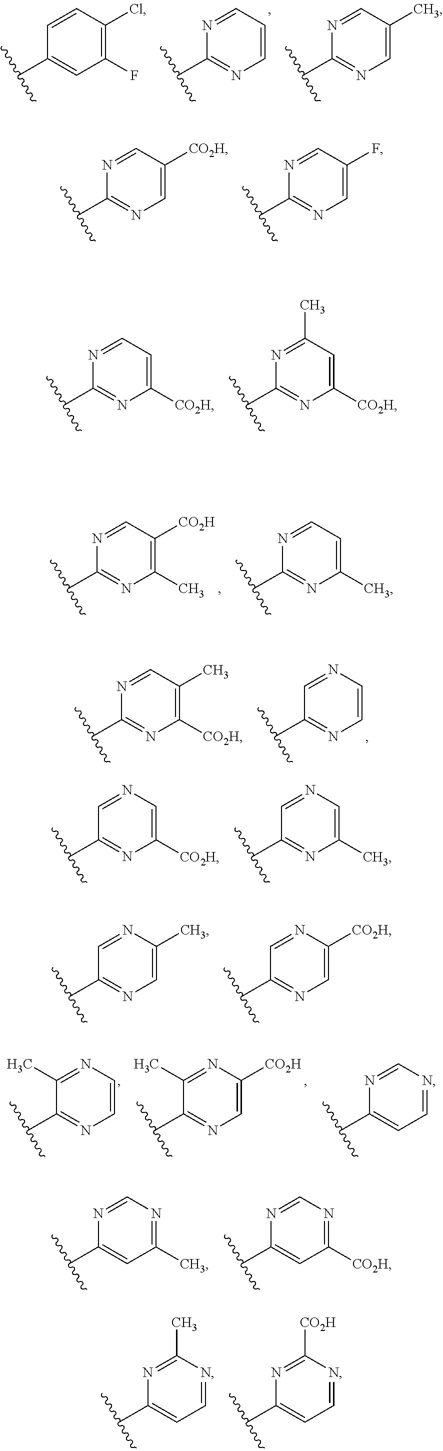

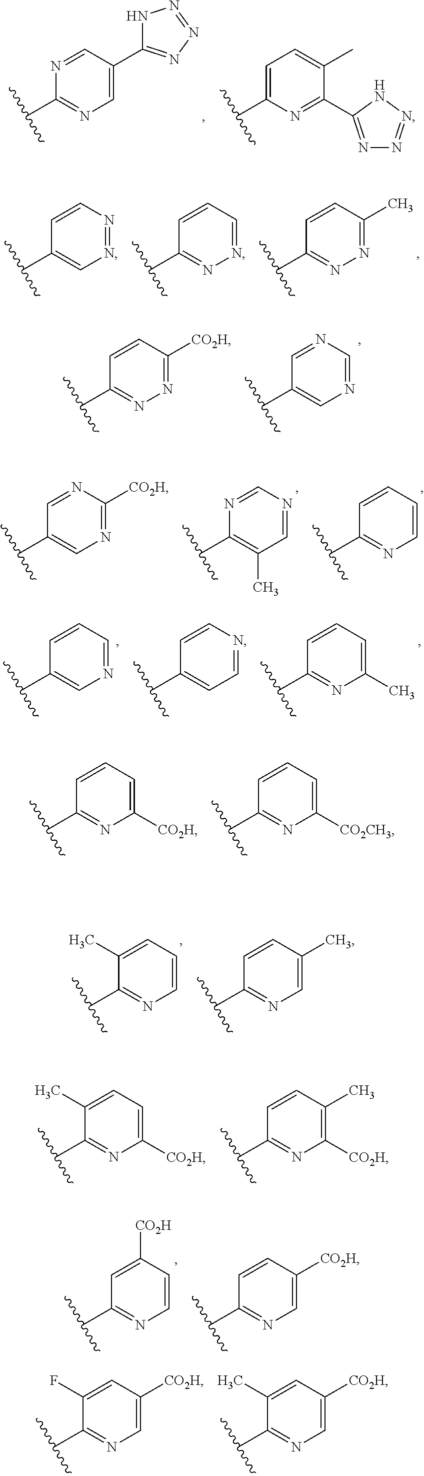

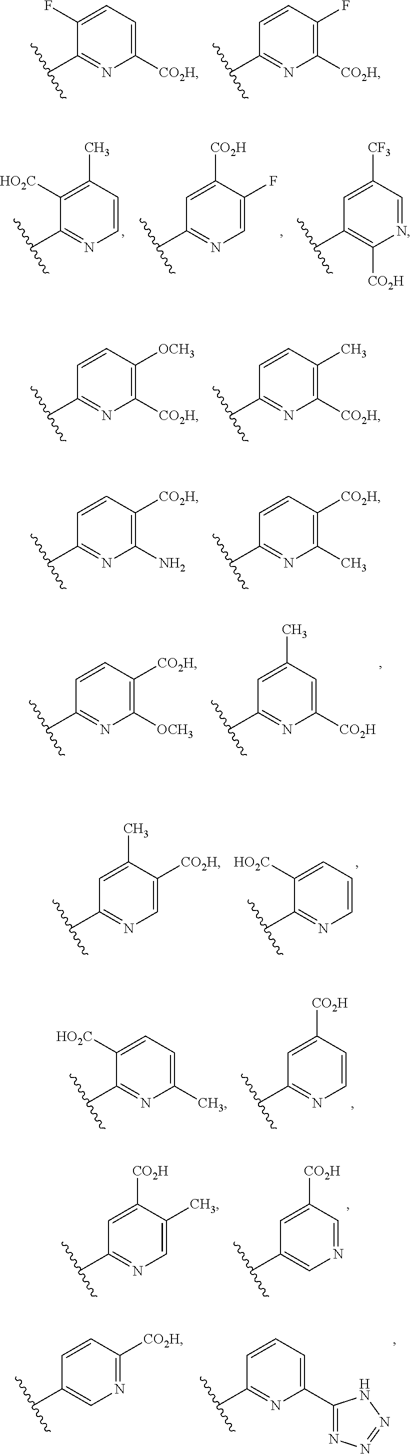

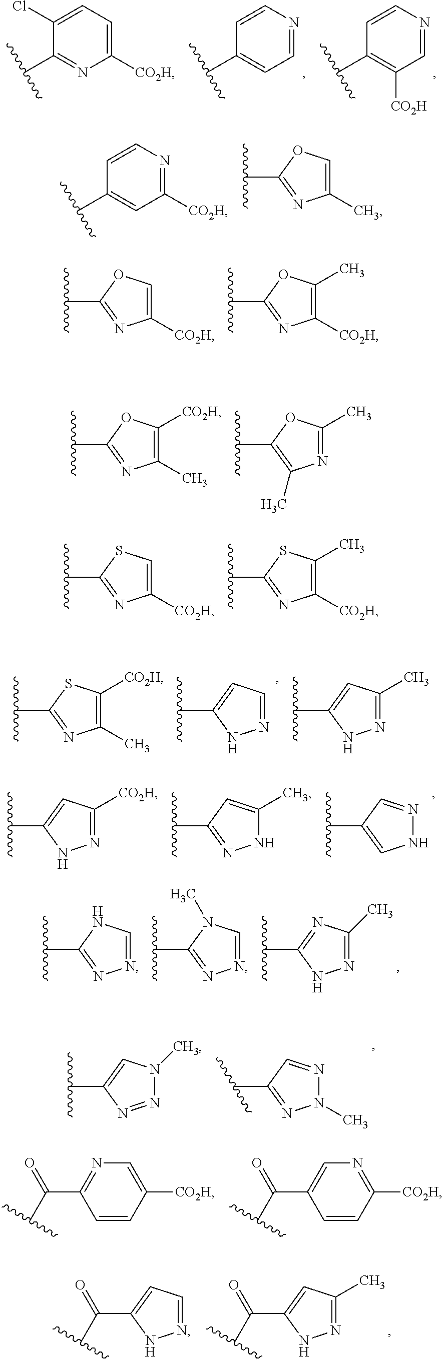

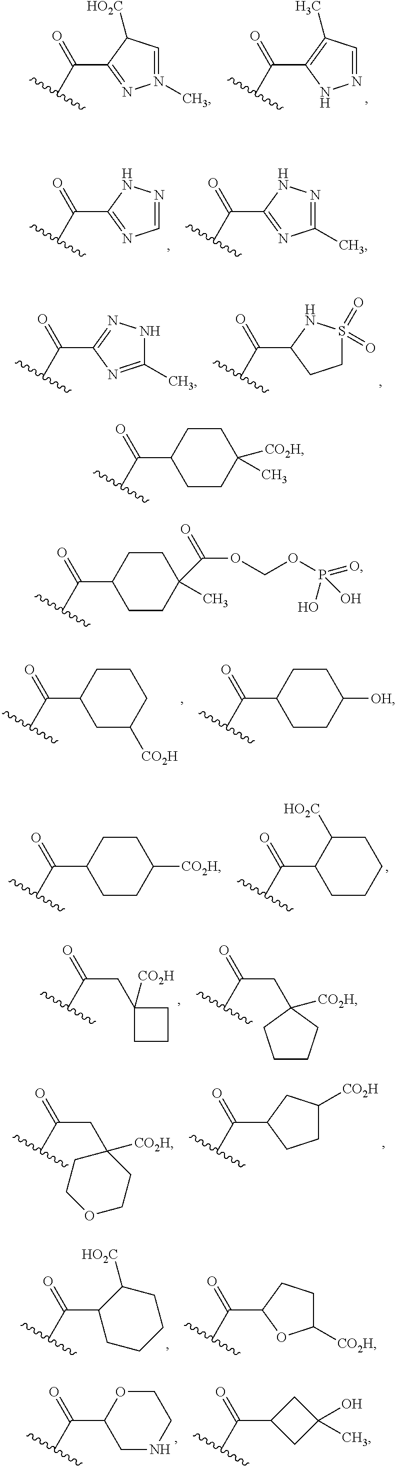

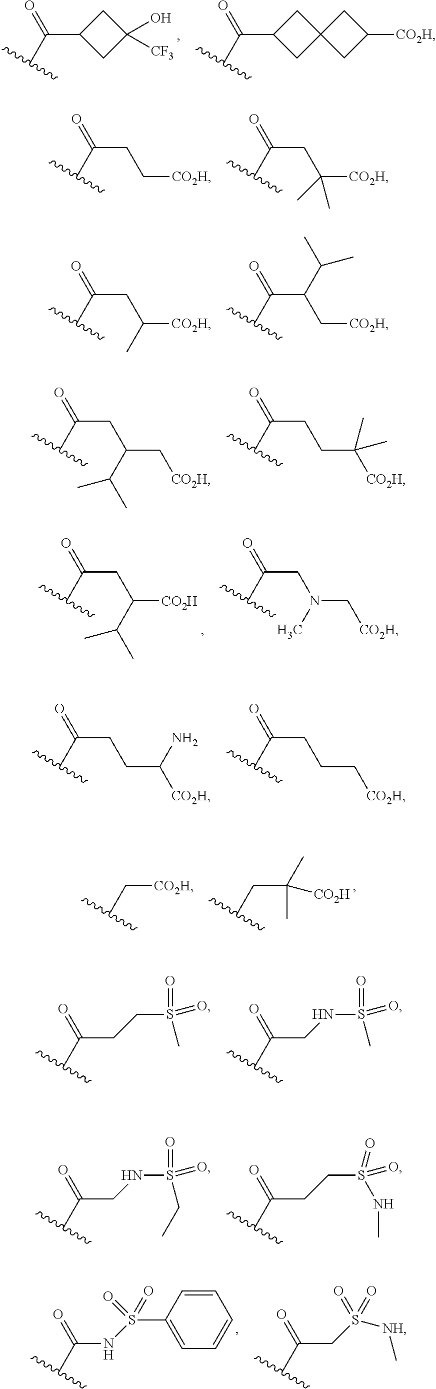

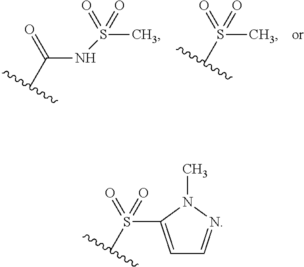

In yet another embodiment, X is: —C(O)CH(CH₃)OH, —C(O)OC(CH₃)₃, —C(O)C(CH₃)₂OH, —C(O)C(OH)(cyclobutyl), —C(O)CH₂CN, —C(O)tetrahydrofuranyl, —C(O)phenyl, —C(O)isoxazolyl, —C(O)CH(OH)CH(CH₃)₂, —C(O)CH(CH₃)₃, —C(O)CH(OH)CH₃, —C(O)C(CH₃)₂F, —C(O)CH₂OCH₃, —C(O)CH(OH)CH₂C(CH₃)₃, —C(O)methylcyclopropyl, —C(O)dimethylcyclopropyl, —C(O)gem dimethylcyclopropyl, —C(O)C═CCH₃, —C(O)CH₂C(CH₃)₃, —C(O)hydroxyl-tetrahydropyranyl, —C(O)CH₃, —C(O)CH(OH)CH₂CH₃, —C(O)CH(OCH₃)CH₃, —C(O)tetrahydrofuranyl. —C(O)CH(OH)cyclopropyl),

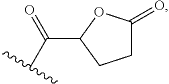 [SMILES: *C(=O)C1CCC(=O)O1]

C(O)CH(OH)(CH₂)₃CH₃, —C(O)C(CH₃)(CH₂CH₃)OH, —C(O)CH(OH)CH₂CH(CH₃)₂, —C(O)CH₂OCH₂CH₂OCH₃, —C(O)CH(OH)CH₂(CH₃)₃, —C(O)C(CH₃)(CF₃)OH, —CH(OH)(cyclohexyl), —CH(OH)CH₂phenyl, —C(O)cyclohexyl, —C(O)(methylcyclohexyl), —C(O)CH(CH₃)CH₂CH₃, —C(O)CH(phenyl)CH₂CH₃, —C(O)cyclobutyl, —C(O)(cyclopropyl), —C(O)(tetramiethylcyclopropyl), —C(O)(cyanocyclopropyl), —C(O)C(CH₃)₂CH₂CH₃,

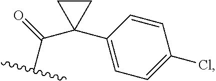 [SMILES: *C(=O)C1(c2ccc(Cl)cc2)CC1]

-
C(O)(tetrahydropyranyl), —C(O)(CF₃-cyclopropyl), —C(O)(hydroxycyclobutyl), —C(O)(hydroxytetrahydropyranyl),

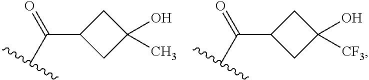

-
C(O)CH₂C(CH₃)₂OH, —C(O)pyrazolyl, —C(O)CH(OCH₃)(phenyl), —C(O)CH(CH₃)(phenyl), —C(O)C(CF₃)(OH)CH₃, —C(O)(OH)CH₂CH₃)₂,

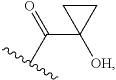 [SMILES: *C(=O)C1(O)CC1]

—C(O)cyclopentyl, —C(O)(difluorocyclopropyl), —C(O)isoxazolyl, —C(O)C(CH₃)₃, —C(O)CH(OH)(4-fluorphenyl), —C(O)OCH₂CH₃, —C(CH₃)₂CH₂OCH₃, or —C(O)dimethylpyrazolyl. In yet another specific embodiment, X is:

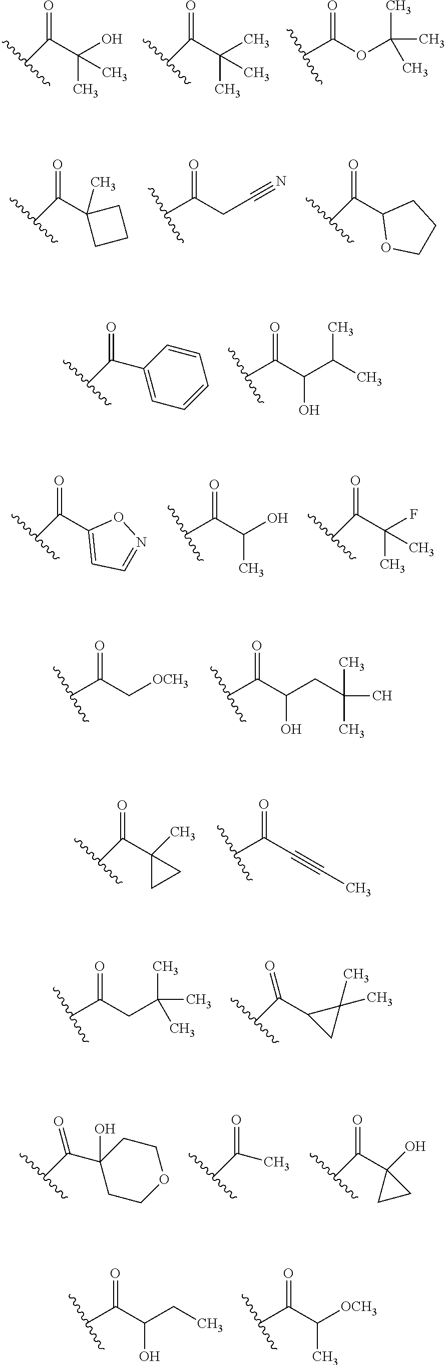

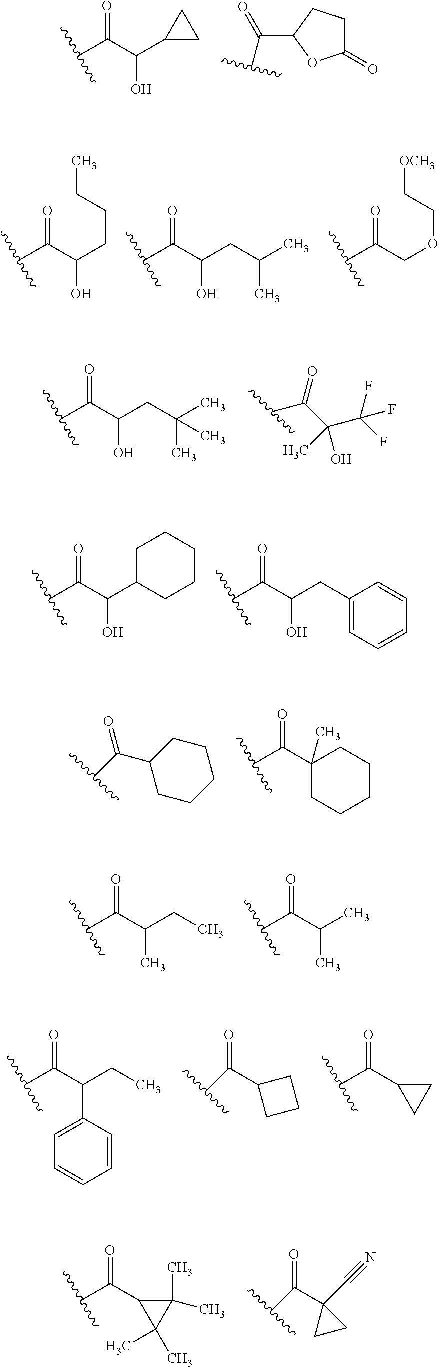

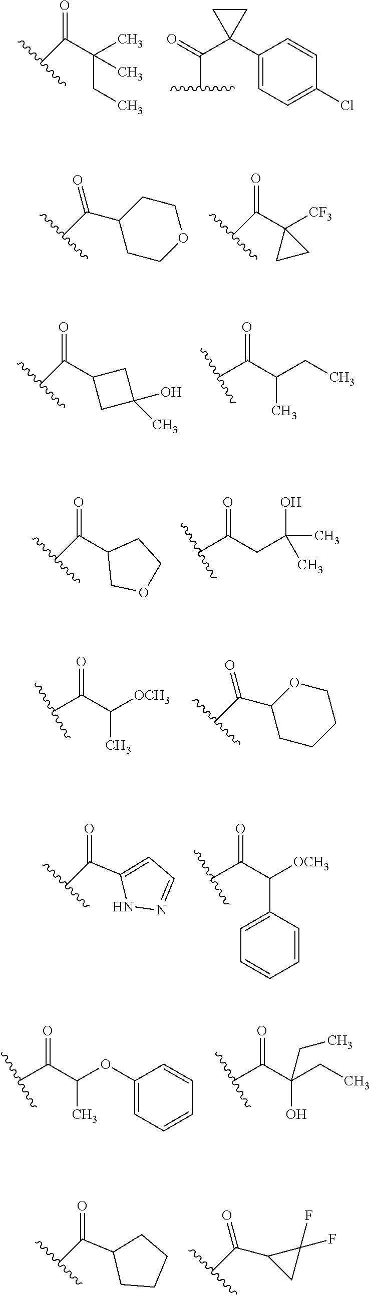

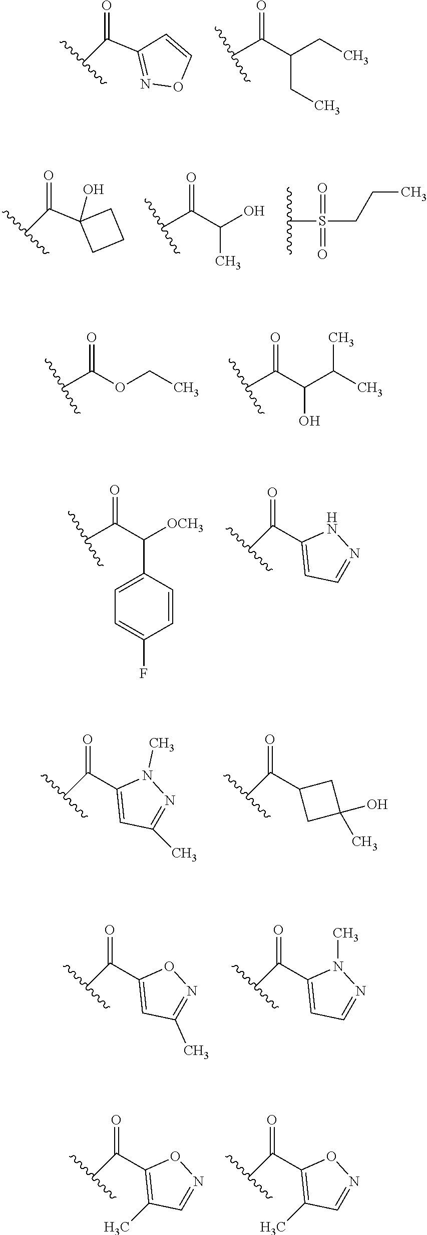

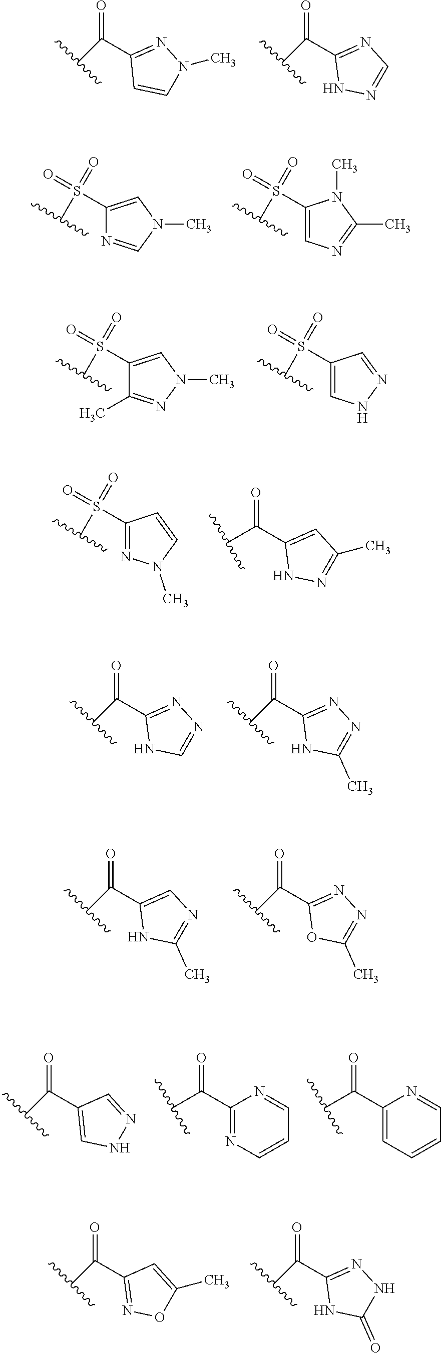

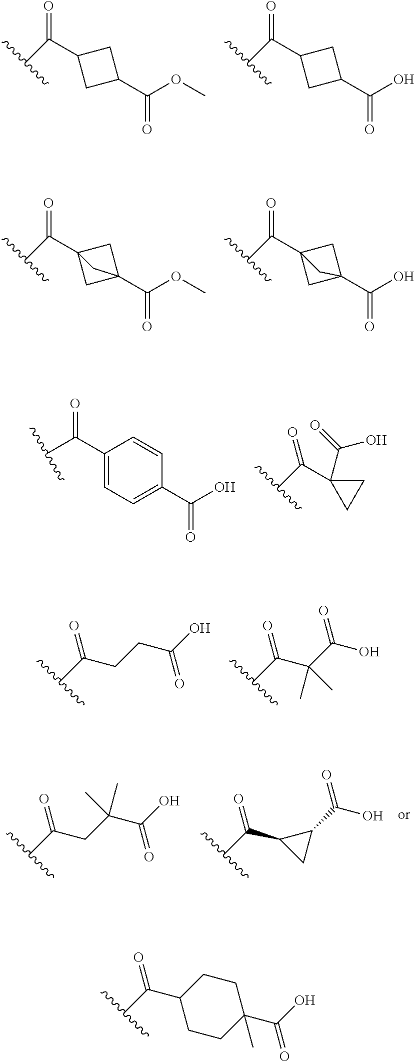

In yet another specific embodiment, X is

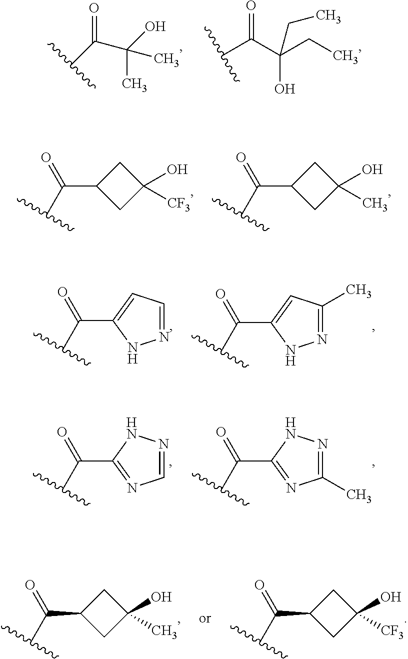

In yet another specific embodiment, X is

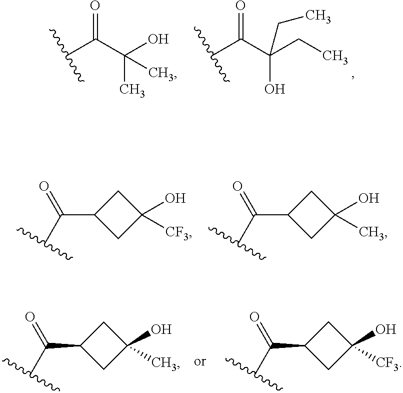

In yet another specific embodiment, X is

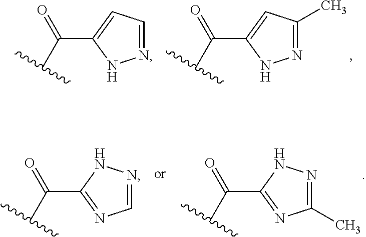

In yet another specific embodiment, X is

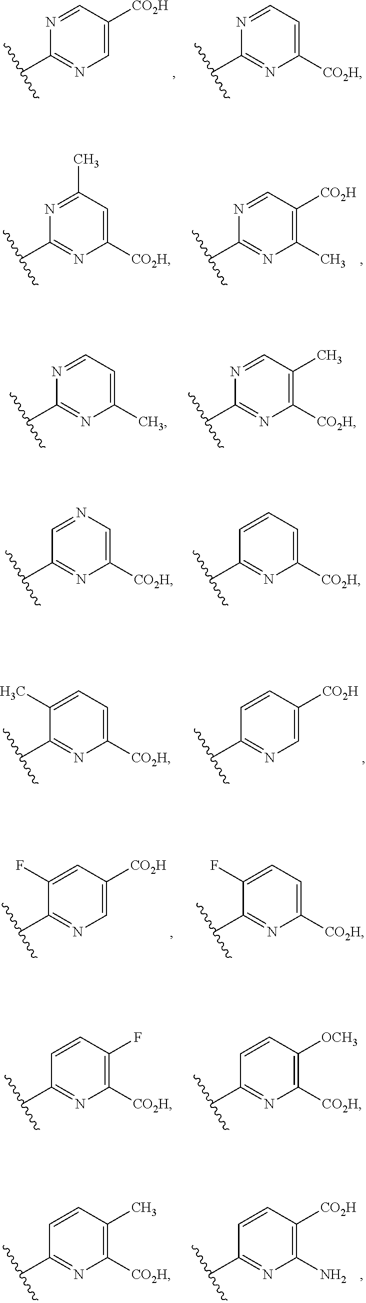

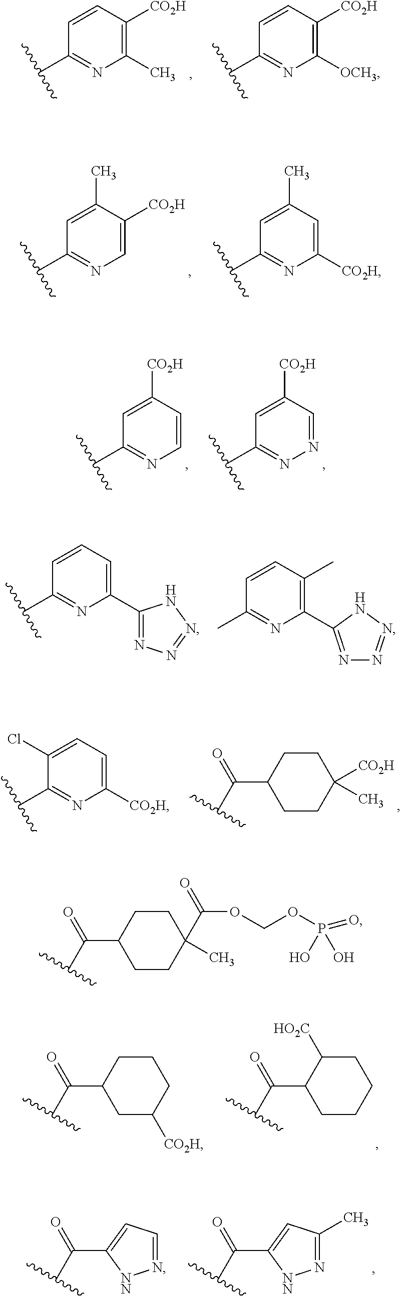

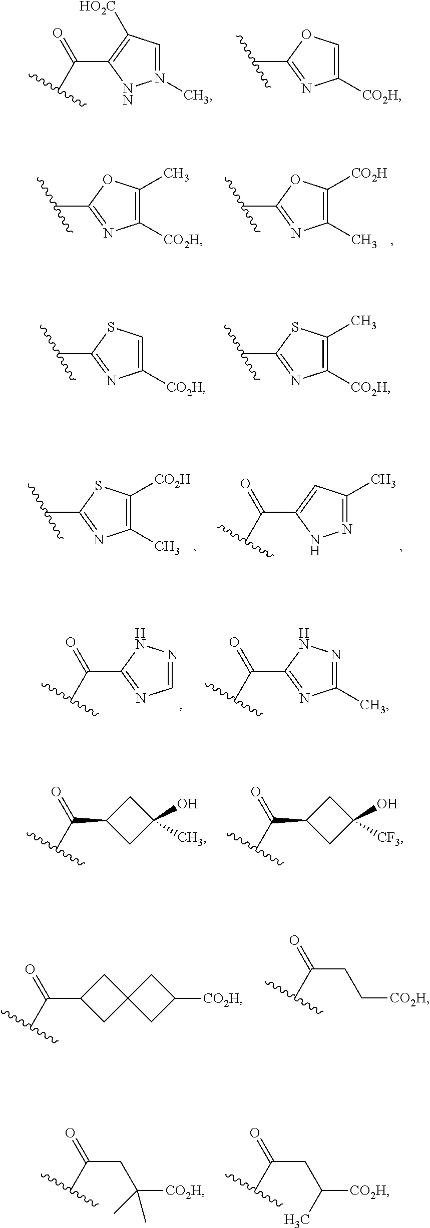

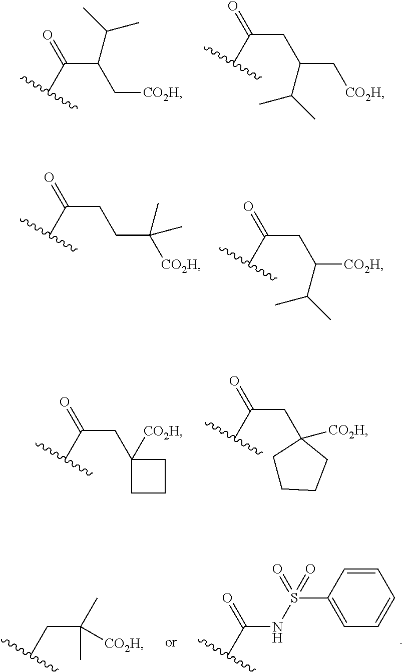

X¹ is R⁵, —C(O)R⁵, or —S(O)₂R⁵.

X² is R⁵.

X³ is —(CR₂)ᵣC(O)OR⁶, —(CR₂)ᵣ—N(R)R⁶, —(CR₂)ᵣC(O)N(R)R⁶ or —(CR₂)—C(O)N(R)S(O)₂R⁶, wherein each r is independently 0, 1, or 2; or optionally X³ and J, together with the atoms to which they are bound, form a 5-6 membered aromatic monocyclic ring having 0-2 heteroatoms selected from oxygen, nitrogen, or sulfur; wherein said 5-6 membered ring forms a fused ring together with Ring A, and is optionally substituted with 1-4 occurrences of substituents selected from oxo, halogen, —CN, —OH, —O(C₁₋₄alkyl), —O(haloC₁₋₄alkyl), C₁₋₄ alkyl, or haloC₁₋₄ alkyl. In one specific embodiment, optionally X³ and J, together with the atoms to which they are bound, form a 5-6 membered aromatic monocyclic ring having 1-2 heteroatoms selected from oxygen, nitrogen, or sulfur; wherein said 5-6 membered ring forms a fused ring together with Ring A. and is optionally substituted with 1-4 occurrences of substituents selected from oxo, halogen, —CN, —OH, —O(C₁₋₄ alkyl), —O(haloC₁₋₄ alkyl), C₁₋₄ alkyl, or haloC₁₋₄ alkyl. Specific examples of 5-6 membered aromatic monocyclic ring that can be formed by X³ and J include phenyl, pyridine, pyrimidine, pyridazine, and pyrazine. In another specific embodiment, X³ is —CR₂—C(O)OR⁶, —N(R)R⁶, —CR₂—C(O)N(R)R⁶ or —CR₂—C(O)N(R)S(O)₂R⁶. In another specific embodiment. X³ is —CR₂—C(O)OR⁶, —N(R)R⁶, —CR₂—C(O)N(R)R⁶ or —CR₂—C(O)N(R)S(O)₂R⁶; and each R⁶ is independently —H or C₁₋₆ alkyl optionally substituted with 1-4 occurrences of substituents selected from CN, halogen, C₁₋₄ alkyl, haloC₁₋₄alkyl, —OH, —O(C₁₋₄alkyl), NH(C₁₋₄alkyl), N(C₁₋₄alkyl)₂, S(O)₂(C₁₋₄alkyl), NH₂, (CH₂)ₜ—C(O)OH, (CH₂)ₜ—C(O)O(C₁₋₂ alkyl), (CH(C₁₋₂ alkyl))ₜ—C(O)OH, (CH(C₁₋₂alkyl))ₜ—C(O)O(C₁₋₂ alkyl), (C(C₁₋₂ alkyl)₂)ₜ—C(O)OH, or (C(C₁₋₂ alkyl)₂)ₜ—C(O)O(C₁₋₂ alkyl), wherein each t is independently 0 or 1. In yet another specific embodiment, X³ is —CH₂—C(O)OH, —NH₂, —NH(C₁₋₆ alkyl), —CH₂—C(O)NH—C₁₋₆ alkyl, —CH₂—C(O)NHS(O)₂C₁₋₆ alkyl, or —CH₂—C(O)NH—CN, wherein each of said C₁₋₆ alkyl is optionally and independently substituted with 1-4 occurrences. In yet another specific embodiment, X³ is —CH₂—C(O)OH, —NH₂, —NH(C₁₋₂alkyl), —CH₂—C(O)NH—C₁₋₂ alkyl, —CH₂—C(O)NHS(O)₂C₁₋₂ alkyl, or —CH₂—C(O)NH—CN, wherein each of said C₁₋₂ alkyl is optionally and independently substituted with 1-4 substitutents selected from CN, halogen, —OH, —OCH₃, or —C(O)OH. In yet another specific embodiment, X³ is —CH₂—C(O)OH, —CH₂—C(O)NH—C₁₋₂alkyl, —CH₂—C(O)NHS(O)₂C₁₋₂ alkyl, or —CH₂—C(O)NH—CN, wherein each of said C₁₋₂ alkyl is optionally and independently substituted with 1-4 substitutents selected from CN, halogen, —OH, —OCH₃, or —C(O)OH.

J is CN, oxo, a C₁₋₆aliphatic group wherein up to three carbon units of said C₁₋₆aliphatic can each be optionally and independently replaced with —O—, —NR—, —S— or —C(O)—; or a 3-7 membered saturated, partially unsaturated, or aromatic monocyclic ring having 0-4 heteroatoms selected from oxygen, nitrogen, or sulfur; wherein said J is optionally and independently substituted with 1-4 occurrences of halogen, CN, or C₁₋₄alkyl, wherein up to one methylene unit of said C₁₋₄alkyl is optionally replaced with —O—, —NR—, or —S—, and wherein said C₁₋₄alkyl is optionally substituted with 1-4 occurrences of halogen or CN; or two J groups on the same or different atom(s), together with the atom(s) to which they are bound, form a 3-6 membered saturated monocyclic ring having 0-2 heteroatoms selected from oxygen, nitrogen, or sulfur; wherein said 3-6 membered ring is optionally substituted with one occurrence of oxo. In a specific embodiment. J is methyl.

J^{A} is C₁₋₄alkyl, J^{B} is C₁₋₄alkyl. J^{C} is methyl. J^{D} is methyl.

R is H or C₁₋₄alkyl.

R² is —(V)_{b}—Y². In one specific embodiment, R² is Y².

R⁴ is halogen; CN; C₁₋₆aliphatic wherein up to three carbon units of said C₁₋₆ aliphatic can each be optionally and independently replaced

with —O—, —NR—, —S— or —C(O)—; a 3-7 membered saturated, partially saturated, or aromatic monocyclic ring having 0-4 heteroatoms selected from oxygen, nitrogen, or sulfur; or a 6-10 membered saturated, partially unsaturated, or aromatic bicyclic ring having 0-6 heteroatoms selected from oxygen, nitrogen, or sulfur; wherein said R⁴ is optionally and independently substituted with 1-4 occurrences of oxo, halogen, CN, or C₁₋₆aliphatic wherein up to three carbon units of said C₁₋₆aliphatic can each be optionally and independently replaced with —O—, —NR—, —S— or —C(O)—. In a specific embodiment, R⁴ is optionally substituted C₁₋₆alkyl, optionally substituted cyclopropyl, or halogen, such as —F or —Cl. In another specific embodiment, the C₁₋₆alkyl is optionally substituted with 1-3 occurrence of —CN, —OCH₃, —OH, or halogen; and the cyclopropyl is optionally substituted with one occurrence of —CN or CH₃. In yet another specific embodiment, R⁴ is C₁₋₆alkyl optionally substituted with 1-3 occurrence of —OCH₃ or halogen, such as —F or —Cl; or cyclopropyl optionally substituted with one occurrence of —CN or —CH₃. In yet another specific embodiment, R⁴ is iso-propyl, tert-butyl, or cyclopropyl optionally substituted with one occurrence of CN or CH₃. In yet another specific embodiment, R⁴ is iso-propyl, tert-butyl, methylcyclopropyl, or cyanocyclopropyl. In yet another specific embodiment, R⁴ is iso-propyl, tert-butyl, methylcyclopropyl, or —C(CH₃)₂CH₂OCH₃.

Each of R⁵ and R⁶ is independently —(V)ₐ—Y. In one embodiment, R⁶ is —H, C₁₋₆ aliphatic or a 3-7 membered saturated, partially unsaturated, or aromatic monocyclic ring having 0-4 heteroatoms selected from oxygen, nitrogen, or sulfur, wherein said C₁₋₆ aliphatic is optionally substituted with 1-4 occurrences of J^{V}, and wherein said monocyclic ring is optionally substituted with 1-4 occurrences of J^{Y}. In another embodiment, R⁶ is —H or C₁₋₆ alkyl optionally substituted with with 1-4 substituents selected from CN, halogen, C₁₋₄alkyl, haloC₁₋₄alkyl, —OH, —O(C₁₋₄alkyl), NH(C₁₋₄alkyl), N(C₁₋₄alkyl)₂, S(O)₂(C₁₋₄alkyl), NH₂, (CH₂)ₜ—C(O)OH, (CH₂)ₜ—C(O)O(C₁₋₂ alkyl), (CH(C₁₋₂ alkyl))ₜ—C(O)OH, (CH(C₁₋₂ alkyl))ₜ—C(O)O(C₁₋₂ alkyl), (C(C₁₋₂ alkyl)₂)ₜ—C(O)OH, or (C(C₁₋₂ alkyl)₂)ₜ—C(O)O(C₁₋₂ alkyl), wherein each t is independently 0, 1 or 2.

V is C₁₋₆aliphatic wherein up to three carbon units of said C₁₋₆aliphatic can each be optionally and independently replaced with —O—, —NR—, —S—, —C(O)—, or —S(O)₂—; wherein V is optionally substituted with 1-4 occurrences of J^{V}. Alternatively, V is C₁₋₆ aliphatic wherein up to three carbon units of said C₁₋₆aliphatic can each be optionally and independently replaced with —O—, —NR—, —S—, or —C(O)—; wherein V is optionally substituted with 1-4 occurrences off J^{V}. In a specific embodiment, V is C₁₋₆aliphatic wherein up to three carbon units of said C₁₋₆aliphatic can each be optionally and independently replaced with —O—, —NR—, —S—, —C(O)—, or —S(O)₂; wherein V is optionally substituted with 1-3 occurrences of halogen, C₁₋₄alkyl, OH, NH₂, or —NRC(O)C₁₋₄alkyl. In yet another specific embodiment, V is C₁₋₆aliphatic wherein up to two carbon units of said C₁₋₆aliphatic can each be optionally and independently replaced with —O— or —C(O)—. In yet another specific embodiment, V is C₁₋₆aliphatic wherein up to two carbon units of said C₁₋₆aliphatic can each be optionally and independently replaced with —O— or —S(O)₂—. In yet another specific embodiment, V is optionally substituted with 1-3 occurrences of J^{V}, wherein J^{V} halogen, CN, C₁₋₄alkyl, OH, O(C₁₋₄alkyl), NH₂, or —NHC(O)C₁₋₄alkyl. In another specific embodiment, V is optionally substituted with 1-3 occurrences of J^{V}, wherein J^{V} is halogen, C₁₋₄alkyl, OH, NH₂, or —NRC(O)C₁₋₄alkyl.

J^{V} is halogen, CN, haloC₁₋₄alkyl, or C₁₋₄alkyl, wherein up to one methylene unit of each of said C₁₋₄alkyl and haloC₁₋₄alkyl is optionally replaced with —O—, —NR—, —S—, or —C(O)—. In one specific embodiment, J^{V} is halogen, CN, or C₁₋₄alkyl, wherein up to one methylene unit of said C₁₋₄alkyl is optionally replaced with —O—, —NR—, or —S—. In another specific embodiment, J^{V} is halogen, CN, C₁₋₄alkyl, OH, O(C₁₋₄alkyl), NH₂, or —NHC(O)C₁₋₄alkyl. In another specific embodiment, J^{V} is halogen, C₁₋₄alkyl, OH, NH₂, or —NRC(O)C₁₋₄alkyl.

Y is H, —CN, a 3-7 membered saturated, partially unsaturated, or aromatic monocyclic ring having 0-4 heteroatoms selected from oxygen, nitrogen, or sulfur; or a 6-10 membered saturated, partially unsaturated, or aromatic bicyclic ring having 0-6 heteroatoms selected from oxygen, nitrogen, or sulfur: wherein Y is optionally substituted with 1-4 occurrences of J^{Y}. In one specific embodiment, Y is H, a 3-7 membered saturated, partially unsaturated, or aromatic monocyclic ring having 0-4 heteroatoms selected from oxygen, nitrogen, or sulfur; or a 6-10 membered saturated, partially unsaturated, or aromatic bicyclic ring having 0-6 heteroatoms selected from oxygen, nitrogen, or sulfur; wherein Y is optionally substituted with 1-4 occurrences of J^{Y}. In another specific embodiment, Y is H or a 3-7 membered saturated, partially unsaturated, or aromatic monocyclic ring having 0-4 heteroatoms selected from oxygen, nitrogen, or sulfur; wherein Y is optionally substituted with 1-4 occurrences of J^{Y}. In another specific embodiment, Y is an optionally substituted, 3-7 membered cycloalkyl or heterocyclic group or an optionally substituted, 5-6 membered aryl or heteroaryl group having 0-4 heteroatoms selected from oxygen, nitrogen, or sulfur. In another specific embodiment, Y is H, cyclopropyl, cyclobutyl, cyclopentyl, cxyclohexyl, oxetanyl, azetidinyl, tetrahydrofuranyl, pyrrolidinyl, tetrahydropyranyl, piperidinyl, piperazinyl, thiomorpholinyl, morpholinyl, imidazolyl, isoxazolyl, pyrazolyl, pyridinyl, pyrazinyl, pyridazinyl, or phenyl: wherein Y is optionally substituted with 1-4 occurrences of J^{Y}. In yet another specific embodiment, Y is H, a 3-6 membered cycloalkyl, isoxazolyl, oxadiazolyl, pyrazolyl, imidazolyl, triazolyl, thienyl, pyrazolyl, tetrahydrofuranyl, tetrahydropyranyl, pyridinyl, pyrimidinyl, or phenyl; wherein Y is optionally substituted with 1-4 occurrences of J^{Y}. In yet another specific embodiment, Y is H, cyclopropyl, cyclobutyl, cyclopentyl, cxyclohexyl, oxetanyl, azetidinyl, tetrahydrofuranyl, pyrrolidinyl, tetrahydropyranyl, piperidinyl, piperazinyl, thiomorpholinyl, morpholinyl, imidazolyl, isoxazolyl, pyrazolyl, pyridinyl, pyrazinyl, pyridazinyl, phenyl, oxadiazolyl, thienyl, pyrimidinyl, triazolyl, tetrazolyl, oxazolyl, thiazolyl, spiro[3,3]hepatanyl, or 1,1-dioxide-isothiazolidinyl; wherein Y is optionally substituted with 1-4 occurrences of J^{Y}. In yet another specific embodiment, Y is H, cyclopropyl, cyclobutyl, cyclopentyl, cyclohexyl, oxetanyl, azetidinyl, tetrahydrofuranyl, pyrrolidinyl, tetrahydropyranyl, piperidinyl, piperazinyl, thiomorpholinyl, morpholinyl, imidazolyl, isoxazolyl, pyrazolyl, pyridinyl, pyrazinyl, pyridazinyl, phenyl, pyrimidinyl, triazolyl, tetrazolyl, oxazolyl, thiazolyl, spiro[3,3]hepatanyl, or 1,1-dioxide-isothiazolidinyl; wherein Y is optionally substituted with 1-4 occurrences of J^{Y}.

J^{Y} is H: oxo: halogen; CN; phenyl; 5-6-membered heteroaryl having 1-4 heteteroatoms selected from oxygen, nitrogen, or sulfur: or C₁₋₆aliphatic, wherein up to three carbon units of said C₁₋₆aliphatic can each be optionally and independently replaced with —O—, —NR—, —S—, —C(O)—, or —S(O)₂—; and wherein each of the phenyl, 5-6 membered heteroaryl and the C₁₋₆aliphatic is optionally and independently substituted with 1-4 occurrences of substituents selected from the group consisting of halogen, —CN, —OH, —OCH₃, —C(O)OH, —OP(O)(OH)₂, —P(O)R(OH), or

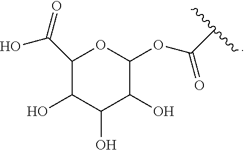 [SMILES: *C(=O)OC1OC(C(=O)O)C(O)C(O)C1O]

In one specific embodiment, J^{Y} is H, oxo, halogen, CN, phenyl, or C₁₋₆aliphatic, wherein up to three carbon units of said C₁₋₆aliphatic can each be optionally and independently replaced with —O—, —NR—, —S—, or —C(O)—; and wherein each of the phenyl and the C₁₋₆aliphatic is optionally and independently substituted with 1-4 occurrences of halogen or CN. In another specific embodiment, J^{Y} is H, oxo, CN, halogen, phenyl, or C₁₋₆aliphatic, wherein up to three carbon units of said C₁₋₆aliphatic can each be optionally and independently replaced with —O—, —NR—, —S— or —C(O)—; and wherein the C₁₋₆aliphatic is optionally substituted with 1-4 halogen. In yet another specific embodiment, J^{Y} is H, oxo, CN, halogen, C₁₋₄alkyl, haloC₁₋₄alkyl, OH, O(C₁₋₄alkyl), NH(C₁₋₄alkyl), N(C₁₋₄alkyl)₂, S(O)₂(C₁₋₄alkyl) or phenyl. In yet another specific embodiment. J^{Y} is oxo, CN, halogen, C₁₋₄alkyl, haloC₁₋₄alkyl, —OH, —O(C₁₋₄alkyl), NH(C₁₋₄alkyl), N(C₁₋₄alkyl)₂, S(O)₂(C₁₋₄alkyl), phenyl, NH₂, tetrazole, (CH₂)ₜ—C(O)OH, (CH₂)ₜ—C(O)O(C₁₋₂ alkyl), (CH(C₁₋₂ alkyl))ₜ—C(O)OH, (CH(C₁₋₂ alkyl))-C(O)O(C₁₋₂ alkyl), (C(C₁₋₂ alkyl)₂)—C(O)OH, (C(C₁₋₂ alkyl)₂)ₜ—C(O)O(C₁₋₂ alkyl), or —C(O)O—CH₂—O—P(O)(OH)₂, wherein each t is independently 0 or 1. In yet another specific embodiment, J^{Y} is —CN, halogen, —CH₃, —CF₃, —OH, —OCH₃, —NH₂, —NHCH₃, N(CH₃)₂, —C(O)OH, or —C(O)O(C₁₋₂ alkyl). In yet another specific embodiment, J^{Y} is H, halogen, CN, C₁₋₄alkyl, haloC₁₋₄alkyl, or OH. In yet another specific embodiment, J^{Y} is H, halogen, C₁₋₄alkyl, or haloC₁₋₄alkyl. In yet another specific embodiment, J^{Y} is H, Cl, F, CH₃, or CF₃.

V² is a C₁₋₄aliphatic group. In a specific embodiment, V² is a C₁₋₄alkyl group.

Y² is halogen; C₁₋₆aliphatic; a 3-7 membered, saturated, partially unsaturated or aromatic, monocyclic ring having 0-4 heteroatoms selected from oxygen, nitrogen, or sulfur: or or a 6-10 membered, saturated, partially unsaturated or aromatic, bicyclic ring having 0-6 heteroatoms selected from oxygen, nitrogen, or sulfur; wherein Y² is optionally substituted with 1-4 occurrences of J^{Y}. In one specific embodiment, Y² is halogen; C₁₋₆aliphatic; or a 3-7 membered saturated, partially unsaturated, or aromatic monocyclic ring having 0-4 heteroatoms selected from oxygen, nitrogen, or sulfur: wherein Y² is optionally substituted with 1-4 occurrences of J^{Y}. In another specific embodiment, Y² is a 3-7 membered saturated, partially unsaturated, or aromatic monocyclic ring having 0-4 heteroatoms selected from oxygen, nitrogen, or sulfur; wherein Y² is optionally substituted with 1-4 occurrences of J^{Y}. In another specific embodiment. Y² is a C₅₋₇ cycloalkyl group or 5-6 membered saturated, partially unsaturated, or aromatic monocyclic ring having 0-4 heteroatoms selected from oxygen, nitrogen, or sulfur: wherein Y² is optionally substituted with 1-4 occurrences of J^{Y}. In yet another specific embodiment, Y² is phenyl optionally substituted with 1-4 occurrences of J^{Y}. In yet another specific embodiment, Y² is a C₅₋₇ cycloalkyl group optionally substituted with 1-4 occurrences of J^{Y}. In yet another specific embodiment, Y² is a non-aromatic, 5-7-membered, heterocycylic ring having 1-2 heteroatoms selected from oxygen, nitrogen, or sulfur, and optionally substituted with 1-4 occurrences of J^{Y}. In yet another specific embodiment, Y² is C₅₋₇ cycloalkyl (e.g., cyclopentyl, cyclohexyl, spiro[3,3]heptanyl), phenyl, or a heterocyclic ring containing 1-2 nitrogen atoms (e.g., pyrrolidine, 6λ²-azaspiro[2.5]octane, 2λ²-azaspiro[3.3]heptane, 1λ²-azetidine, or piperidine), wherein Y² is optionally substituted with 1-4 occurrences of J^{Y}. In yet another specific embodiment, Y² is cyclopentyl, cyclohexyl,

spiro[3,3]heptanyl, phenyl,

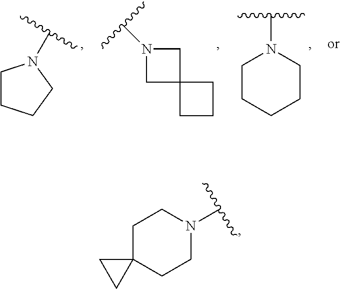

wherein Y is optionally substituted with 1-4 occurrences of J^{Y}. In some embodiments, J^{Y} for Y² is halogen, CN, or C₁₋₄alkyl. In some embodiments, J^{Y} for Y² is halogen or C₁₋₄alkyl. In some embodiments, J^{Y} for Y² is halogen.

Each of a and b is independently 0 or 1. In a specific embodiment, b is 0.

n is 1 or 2.

p is 0-4. In a specific embodiment, p is 1 or 2.

t is 0, 1 or 2.

The aforementioned monocyclic ring and bicyclic rings for J, R⁴, Y, and Y² can each independently be cycloalphatic, heterocyclic, aryl or heteroary groups. In some embodiments, monocyclic ring and bicyclic rings are cycloalkyl, heterocyclic, aryl or heteroaryl groups.

In the second set of variables of formula (I). Z is —NX—; and the other variables of formula (I) are each and independently as described above in the first set of variables of formula (I).

In the third set of variables of formula (I), Z is —NX—; J is methyl; and the other variables of formula (I) are each and independently as described above in the first set of variables of formula (I).

In the fourth set of variables of formula (I), Z is —NX—; p is 1 or 2; and the other variables of formula (I) are each and independently as described above in the first set of variables of formula (I).

In the fifth set of variables of formula (I), Z is —NX—; J is methyl; p is 1 or 2; and the other variables of formula (I) are each and independently as described above in the first set of variables of formula (I).

In the sixth set of variables of formula (I), Z, J and p are each independently as defined in any of the second through fifth sets of variables of formula (I); n is 1; and the other variables of formula (I) are each and independently as described above in the first set of variables of formula (I).

In the seventh set of variables of formula (I), Ring A is

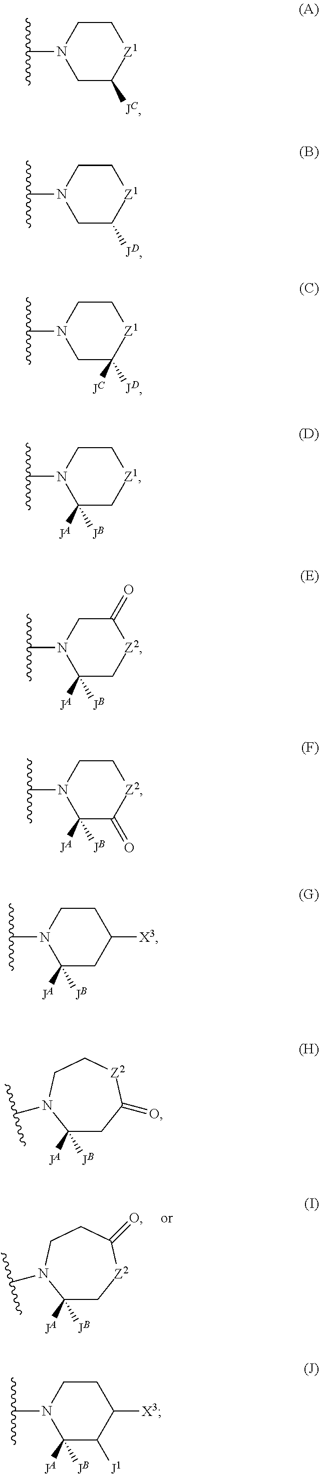

wherein
each Z¹ is independently-O—, —CH₂—, or —NX—; wherein X is X¹;
each Z² is independently —CH₂— or —NX—; wherein X is X²;
X¹ is R⁵, —C(O)R⁵, or —S(O)₂R⁵;
X² is R⁵;
J^{A} is C₁₋₄alkyl;
J^{B} is C₁₋₄alkyl;
or J^{A} and J^{B}, together with the carbon atom to which they are bound, form a 3-6 membered saturated monocyclic ring having 0-1 heteroatom selected from oxygen, nitrogen, or sulfur; and
X³ in formula (G) is —(CR₂)ᵣC(O)OR⁶, —(CR₂)ᵣ—N(R)R⁶, —(CR₂)ᵣC(O)N(R)R⁶ or —(CR₂)ᵣ—C(O)N(R)S(O)₂R⁶;
each r is independently 0, 1 or 2:
J¹ and X³ in formula (J), together with the atoms to which they are bound, form a 5-6 membered, aromatic, monocyclic ring having 1-2 heteroatoms selected from oxygen, nitrogen, or sulfur, wherein said 5-6 membered ring forma a fused ring together with Ring A. and is optionally substituted with 1-4 occurrences of substituents selected from oxo, halogen, CN. —OH, —O(C₁₋₄ alkyl), —O(haloC₁₋₄ alkyl), C₁₋₄ alkyl, or haloC₁₋₄alkyl;
each R⁶ is —H, C₁₋₆ aliphatic or a 3-7 membered saturated, partially unsaturated, or aromatic monocyclic ring having 0-4 heteroatoms selected from oxygen, nitrogen, or sulfur, wherein said C₁₋₆ aliphatic is optionally substituted with 1-4 occurrences of J^{V}, and wherein said monocyclic ring is optionally substituted with 1-4 occurrences of J^{Y};
J^{C} is methyl; and
J^{D} is methyl.

The other variables are each and independently as described above in any one of the first through sixth sets of variables of formula (I).

In the eighth set of variables of formula (I), Ring A is

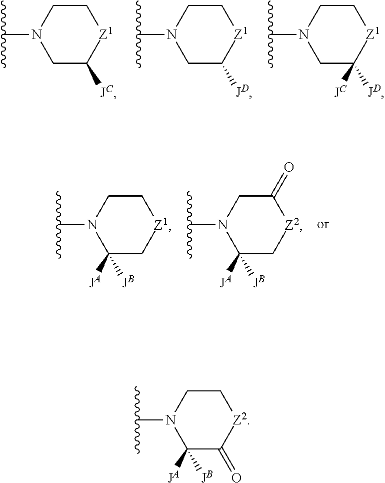

Each Z¹ is independently —O—, —CH₂—, or —NX—, wherein X is X¹. Each Z² is independently —CH₂— or —NX—, wherein X is X². X¹ is R⁵, —C(O)R⁵, or —S(O)₂R⁵. X² is R⁵. R⁵ is —(V)ₐ—Y. V is C₁₋₆aliphatic wherein up to three carbon units of said C₁₋₆aliphatic can each be optionally and independently replaced
with —O—, —NR—, —S—, or —C(O)—, wherein V is optionally substituted with 1-3 occurrences of halogen. C₁₋₄alkyl, OH, NH₂, or —NRC(O)C₁₋₄alkyl. Y is H or a 3-7 membered saturated, partially unsaturated, or aromatic monocyclic ring having 0-4 heteroatoms selected from oxygen, nitrogen, or sulfur, wherein Y is optionally substituted with 1-4 occurrences of J^{Y}. J^{Y} is H, oxo, CN, halogen, phenyl, or C₁₋₆aliphatic, wherein up to three carbon units of said C₁₋₆aliphatic can each be optionally and independently replaced with —O—, —NR—, —S— or —C(O)—, wherein the C₁₋₆aliphatic group is optionally substituted with 1-4 occurrences of halogen. R is H or C₁₋₄alkyl. b is 0 or 1. J^{A} is C₁₋₄alkyl; J^{B} is C₁₋₄alkyl; or J^{A} and J^{B}, together with the carbon atom to which they are bound, form a 3-6 membered saturated monocyclic ring having 0-1 heteroatom selected from oxygen, nitrogen, or sulfur. J^{C} is methyl. J^{D} is methyl. The other variables are each and independently as described above in any one of the first through sixth sets of variables of formula (I).

In the ninth set of variables of formula (I), Ring A is

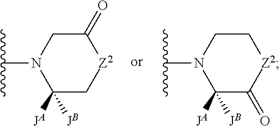

and the other variables of formula (I) are each and independently as described above in any one of the first through eighth sets of variables of formula (I).

In the tenth set of variables of formula (I), Ring A is

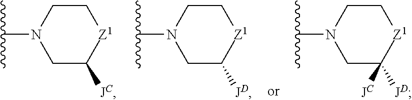

and the other variables of formula (I) are each and independently as described above in any one of the first through eighth sets of variables of formula (I).

In the eleventh set of variables of formula (I), Ring A is

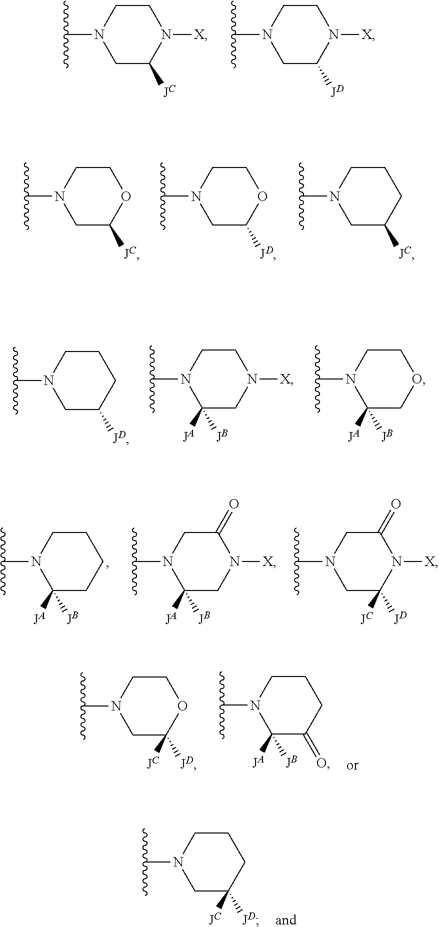

the other variables of formula (I) are each and independently as described above in any one of the first through the eighth sets of variables of formula (I). Alternatively,

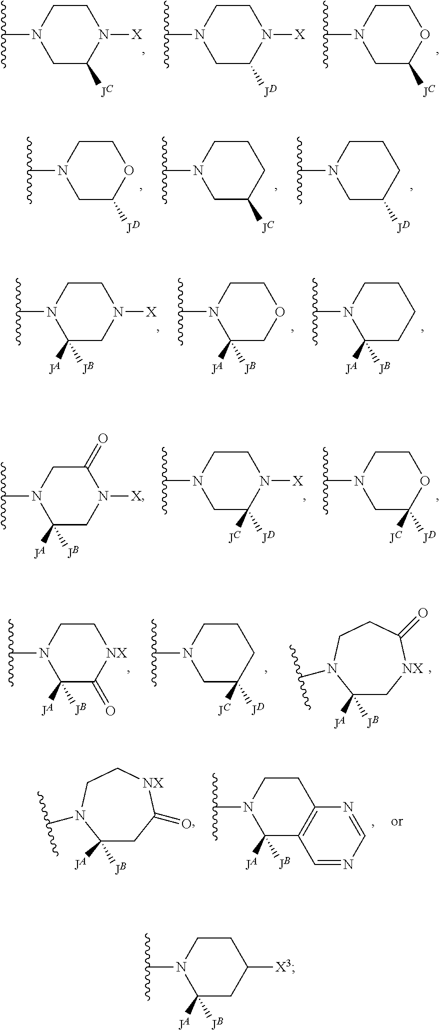

the other variables of formula (I) are each and independently as described above in any one of the first through the eighth sets of variables of formula (I).

In the twelfth set of variables of formula (I), Ring A is

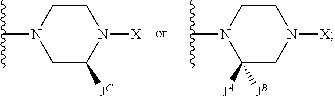

and the other variables of formula (I) are each and independently as described above in any one of the first through eighth sets of variables of formula (I).

In the thirteenth set of variables of formula (I), X is —R⁵ or C(O)R⁵; Ring A is as defined in any one of the ninth through twelfth sets of variables of formula (I); and the other variables of formula (I) are each and independently as described above in the first set of variables of formula (I).

In the fourteenth set of variables of formula (I), X is —R⁵ or C(O)R; Ring A is as defined in any one of the ninth through twelfth sets of variables of formula (I); and the other variables of formula (I) are each and independently as described above in the first through eighth sets of variables of formula (I).

In the fifteenth set of variables of formula (I), X is —R⁵ or C(O)R⁵; Ring A is as defined in any one of the ninth through twelfth sets of variables of formula (I): R⁵ is —(V)ₐ—Y: V is C₁₋₆aliphatic wherein up to three carbon units of said C₁₋₆aliphatic can each be optionally and independently replaced with —O—, —NR—, —S—, or —C(O)—, wherein V is optionally substituted with 1-3 occurrences of halogen, C₁₋₄alkyl, OH, NH₂, or —NRC(O)C₁₋₄alkyl; Y is H, cyclopropyl, cyclobutyl, cyclopentyl, cxyclohexyl, oxetanyl, azetidinyl, tetrahydrofuranyl, pyrrolidinyl, tetrahydropyranyl, piperidinyl, piperazinyl, thiomorpholinyl, morpholinyl, imidazolyl, isoxazolyl, pyrazolyl, pyridinyl, pyrazinyl, pyridazinyl, or phenyl, wherein Y is optionally substituted with 1-4 occurrences of J^{Y}; wherein J^{Y} is H, oxo, CN, halo, C₁₋₄alkyl, haloC₁₋₄alkyl, OH, O(C₁₋₄alkyl), NH(C₁₋₄alkyl), N(C₁₋₄alkyl)₂, S(O)₂(C₁₋₄alkyl) or phenyl; a is 0 or 1; and the other variables of formula (I) are each and independently as described above in any one of the first through eighth sets variables of formula (I).

In the sixteenth set of variables of formula (I), X is —R⁵ or C(O)R⁵; Ring A is as defined in any one of the ninth through twelfth sets of variables of formula (I); R⁵ is —(V)ₐ—Y: V is C₁₋₆aliphatic wherein up to three carbon units of said C₁₋₆aliphatic can each be optionally and independently replaced with —O—, —NR—, —S—, or —C(O)—, wherein V is optionally substituted with 1-3 occurrences of halogen, C₁₋₄alkyl, OH, NH₂, or —NRC(O)C₁₋₄alkyl; Y is H, cyclopropyl, cyclobutyl, cyclopentyl, cxyclohexyl, oxetanyl, azetidinyl, tetrahydrofuranyl, pyrrolidinyl, tetrahydropyranyl, piperidinyl, piperazinyl, thiomorpholinyl, morpholinyl, imidazolyl, isoxazolyl, pyrazolyl, pyridinyl, pyrazinyl, pyridazinyl, or phenyl, wherein Y is optionally substituted with 1-4 occurrences of J^{Y}; wherein J^{Y} is H, oxo, CN, halo, C₁₋₄alkyl, haloC₁₋₄alkyl, OH, O(C₁₋₄alky), NH(C₁₋₄alkyl), N(C₁₋₄alkyl)₂, S(O)₂(C₁₋₄alkyl) or phenyl; a is 0 or 1; and the other variables of formula (I) are each and independently as described above in any one of the first through eighth sets of variables of formula (I).

In the seventeenth set of variables of formula (I), Ring A is as defined in the seventh or eleventh set of variables of formula (I); X³ is —CR₂—C(O)OR⁶, —N(R)R⁶. —CR₂—C(O)N(R)R⁶ or —CR₂—C(O)N(R)S(O)₂R⁶; and the other variables of formula (I) are each and independently as described above in the seventh or eleventh set of variables of formula (I).

In the eighteenth set of variables of formula (I), Ring A is as defined in the seventh or eleventh set of variables of formula (I); X³ is —CH₂—C(O)OH. —NH₂, —NH(C₁₋₆ alkyl), —CH₂—C(O)NH—C₁₋₆ alkyl, —CH₂—C(O)NHS(O)₂C₁₋₆ alkyl, or —CH₂—C(O)NH—CN, wherein each of said C₁₋₆ alkyl is optionally and independently substituted with 1-4 occurrences of substituents selected from CN, halogen, C₁₋₄alkyl, haloC₁₋₄alkyl, —OH, —O(C-₁₋₄alkyl), NH(C₁₋₄alkyl), N(C₁₋₄alkyl)₂, S(O)₂(C₁₋₄alkyl), NH₂, (CH₂)—C(O)OH, (CH₂)ₜ—C(O)O(C₁₋₂ alkyl), (CH(C₁₋₂ alkyl))ₜ—C(O)OH, (CH(C₁₋₂ alkyl)), —C(O)O(C₁₋₂ alkyl), (C(C₁₋₂ alkyl)₂)ₜ—C(O)OH, or (C(C₁₋₂ alkyl)₂)ₜ—C(O)O(C₁₋₂ alkyl), wherein each t is independently 0 or 1; and the other variables of formula (I) are each and independently as described above in the seventh or eleventh set of variables of formula (I).

In the nineteenth set of variables of formula (I):
X is C₁₋₆alkyl, —Y, —C(O)—C₁₋₆ alkyl, —S(O)₂—C₁₋₃ alkyl, —C(O)—(CH₂)_{q}—C₁₋₃ alkyl, —C(O)N(R)S(O)₂—C₁₋₃ alkyl, —C(O)—(CH₂)_{q}—N(R)S(O)₂—C₁ alkyl, —C(O)N(R)S(O)₂—C₁₋₃ alkyl, —C(O)—(CH₂)_{q}—S(O)₂—N(R)—C₁₋₂ alkyl, —C(O)N(R)—C₁₋₃ alkyl, —C(O)—(CH₂)_{q}—S(O)₂—C₁₋₃ alkyl, —C(O)Y, —S(O)₂Y, —C(O)—(CH₂)_{q}—Y, —C(O)N(R)S(O)₂Y, —C(O)—(CH₂)_{q}—N(R)S(O)₂Y, —C(O)N(R)S(O)₂Y, —C(O)—(CH₂)_{q}—S(O)₂—N(R)Y, —C(O)N(R)Y, —C(O)—(CH₂)_{q}—S(O)₂Y, wherein each q is independently 1 or 2;
wherein each of said C₁₋₆ alkyl, C₁₋₃ alkyl and C₁₋₂ alkyl is optionally and independently substituted with 1-4 occurrences of J^{V} selected from CN, halogen, C₁₋₄alkyl, haloC₁₋₄alkyl, —OH, —O(C₁₋₂alkyl), —NH(C₁₋₂alkyl), —N(C₁₋₂alkyl)₂, —S(O)₂(C₁₋₂alkyl), —NH₂, —C(O)OH, —CH₂—C(O)OH. —(CH₂)₂—C(O)OH, —CH₂—C(O)OCH₃, or —C(O)O(C₁₋₂ alkyl);

each Y is optionally substituted with 1-4 occurrences of J^{Y} selected from oxo, CN, halogen, C₁₋₄alkyl, haloC₁₋₄alkyl, —OH, —O(C₁₋₄alkyl), NH(C₁₋₄alkyl), N(C₁₋₄alkyl)₂, S(O)₂(C₁₋₄alkyl), phenyl, NH₂, tetrazole, (CH₂)ₜ—C(O)OH, (CH₂)ₜ—C(O)O(C₁₋₂ alkyl), (CH(C₁₋₂ alkyl))ₜ—C(O)OH, (CH(C₁₋₂ alkyl))ₜ—C(O)O(C₁₋₂ alkyl), (C(C₁₋₂ alkyl)₂)ₜ—C(O)OH, (C(C₁₋₂ alkyl)₂)ₜ—C(O)O(C₁₋₂ alkyl), or —C(O)O—CH₂—O—P(O)(OH)₂, wherein each t is independently 0 or 1; and

the other variables of formula (I) are each and independently as described above in any one of the first through eighteenth sets of variables of formula (I).

In the twentieth set of variables of formula (I), Y is H, cyclopropyl, cyclobutyl, cyclopentyl, cyclohexyl, oxetanyl, azetidinyl, tetrahydrofuranyl, pyrrolidinyl, tetrahydropyranyl, piperidinyl, piperazinyl, thiomorpholinyl, morpholinyl, imidazolyl, isoxazolyl, pyrazolyl, pyridinyl, pyrazinyl, pyridazinyl, phenyl, pyrimidinyl, triazolyl, tetrazolyl, oxazolyl, thiazolyl, spiro[3,3]hepatanyl, or 1,1-dioxide-isothiazolidinyl; wherein Y is optionally substituted with 1-4 occurrences of J^{Y}; and the other variables of formula (I) are each and independently as described above in any one the first through nineteenth sets of variables of formula (I).

In the twenty first set of variables of formula (I), Y is H, cyclopropyl, cyclobutyl, cyclopentyl, cyclohexyl, oxetanyl, azetidinyl, tetrahydrofuranyl, pyrrolidinyl, tetrahydropyranyl, piperidinyl, piperazinyl, thiomorpholinyl, morpholinyl, imidazolyl, isoxazolyl, pyrazolyl, pyridinyl, pyrazinyl, pyridazinyl, phenyl, pyrimidinyl, triazolyl, tetrazolyl, oxazolyl, thiazolyl, spiro[3,3]hepatanyl, or 1,1-dioxide-isothiazolidinyl; wherein Y is optionally substituted with 1-4 occurrences of J^{Y}; J^{Y} is —CN, halogen, —CH₃, —CF₃, —OH, —OCH₃, —NH₂, —NHCH₃, N(CH₃)₂, —C(O)OH, or —C(O)O(C₁₋₂ alkyl); and J^{V} is —CN, halogen, —CH₃, —CF₃, —OH, —OCH₃, —NH₂, —NHCH₃, N(CH₃)₂, —C(O)OH, or —C(O)O(C₁₋₂ alkyl); and the other variables of formula (I) are each and independently as described above in any one the first through nineteenth sets of variables of formula (I).

In the twenty second set of variables of formula (I): X is —R⁵ or C(O)R⁵: Ring A is as defined in any one of the ninth through twelfth sets of variables of formula (I); R⁵ is —(V)ₐ—Y: V is C₁₋₆aliphatic wherein up to three carbon units of said C₁₋₆aliphatic can each be optionally and independently replaced with —O—, —NR—, —S—, —C(O)—, or —S(O)₂; wherein V is optionally substituted with 1-3 occurrences of halogen, C₁₋₄alkyl, OH, NH₂, or —NRC(O)C₁₋₄alkyl; and Y is H, cyclopropyl, cyclobutyl, cyclopentyl, cxyclohexyl, oxetanyl, azetidinyl, tetrahydrofuranyl, pyrrolidinyl, tetrahydropyranyl, piperidinyl, piperazinyl, thiomorpholinyl, morpholinyl, imidazolyl, isoxazolyl, pyrazolyl, pyridinyl, pyrazinyl, pyridazinyl, phenyl, oxadiazolyl, thienyl, pyrimidinyl, triazolyl, tetrazolyl, oxazolyl, thiazolyl, spiro[3,3]hepatanyl, or 1,1-dioxide-isothiazolidinyl; wherein Y is optionally substituted with 1-4 occurrences of J^{Y}; J^{Y} is H, oxo, CN, halogen, C₁₋₄alkyl, haloC₁₋₄alkyl, —OH, —O(C₁₋₄alkyl), NH(C₁₋₄alkyl), N(C₁₋₄alkyl)₂, S(O)₂(C₁₋₄alkyl), phenyl, NH₂, tetrazole, (CH₂)ₜ—C(O)OH, (CH₂)ₜ—C(O)O(C₁₋₂ alkyl), (CH(C₁₋₂ alkyl))ₜ—C(O)OH, (CH(C₁₋₂ alkyl))ₜ—C(O)O(C₁₋₂ alkyl), (C(C₁₋₂ alkyl)₂)ₜ—C(O)OH, (C(C₁₋₂ alkyl)₂)ₜ—C(O)O(C₁₋₂alkyl), or —C(O)O—CH₂—O—P(O)(OH)₂: a is 0 or 1: each t is independently 0 or 1; and the other variables of formula (I) are each and independently as described above in any one the first through nineteenth sets of variables of formula (I).

In the twenty third set of variables of formula (I): X is —R⁵ or C(O)R⁵; Ring A is as defined in any one of the ninth through twelfth sets of variables of formula (I); R⁵ is —(V)ₐ—Y; V is C₁₋₆aliphatic wherein up to three carbon units of said C₁₋₆aliphatic can each be optionally and independently replaced with —O—, —NR—, —S—, or —C(O)—; wherein V is optionally substituted with 1-3 occurrences of halogen, C₁₋₄alkyl, OH, NH₂, or —NRC(O)C₁₋₄alkyl; and Y is H, cyclopropyl, cyclobutyl, cyclopentyl, cxyclohexyl, oxetanyl, azetidinyl, tetrahydrofuranyl, pyrrolidinyl, tetrahydropyranyl, piperidinyl, piperazinyl, thiomorpholinyl, morpholinyl, imidazolyl, isoxazolyl, pyrazolyl, pyridinyl, pyrazinyl, pyridazinyl, or phenyl; wherein Y is optionally substituted with 1-4 occurrences of J^{Y}; J^{Y} is H, oxo, CN, halogen, C₁₋₄alkyl, haloC₁₋₄alkyl, OH, O(C₁₋₄alkyl), NH(C₁₋₄ alkyl), N(C₁₋₄alkyl)₂, or phenyl: a is 0 or 1; and the other variables of formula (I) are each and independently as described above in any one the first through nineteenth sets of variables of formula (I).

In the twenty fourth set of variables of formula (I), R² is —(V²)_{b}—Y²; b is 0; and the other variables are each and independently as described above in any one of the first through twenty third sets of variables of formula (I).

In the twenty fifth set of variables of formula (I), R² is —(V²)_{b}—Y²; b is 0: Y² is a C₅₋₇ cycloalkyl group or 5-6 membered saturated, partially unsaturated, or aromatic monocyclic ring having 0-4 heteroatoms selected from oxygen, nitrogen, or sulfur, wherein Y² is optionally substituted with 1-4 occurrences of J^{Y}; and the other variables are each and independently as described above in any one of the first through twenty third sets of variables of formula (I).

In the twenty sixth set of variables of formula (I), R² is —(V²)_{b}—Y²; b is 0; Y² is phenyl optionally substituted with 1-4 occurrences of halogen; and the other variables are each and independently as described above in any one of the first through twenty third sets of variables of formula (I).

In the twenty seventh set of variables of formula (I), R² is —(V²)_{b}—Y²; b is 0: Y² is a C₅₋₇ cycloalkyl group optionally substituted with 1-4 occurrences of halogen or C₁₋₄alkyl; and the other variables are each and independently as described above in any one of the first through twenty third sets of variables of formula (I).

In the twenty eighth set of variables of formula (I). R² is —(V²)_{b}—Y²; b is 0; Y² is a non-aromatic, 5-7-membered, heterocycylic ring having 1-2 heteroatoms selected from oxygen, nitrogen, or sulfur, and optionally substituted with 1-4 occurrences of halogen or C₁₋₄alkyl; and the other variables are each and independently as described above in any one of the first through twenty third sets of variables of formula (I).

In the twenty ninth set of variables of formula (I), R⁴ is optionally substituted C₁₋₆alkyl, optionally substituted cyclopropyl, or halogen, such as —F, or —Cl, wherein specific examples of substituents for the C₁₋₆alkyl and cyclopropyl are independently as described in the first set of variables of formula (I); and the other variables are each and independently as described above in any one of the first through the twenty third sets of variables of formula (I).

In the thirtieth set of variables of formula (I). R² is —(V²)_{b}—Y²; b is 0; R⁴ is optionally substituted C₁₋₆alkyl, optionally substituted cyclopropyl, or halogen, such as —F, or —Cl, wherein specific examples of substituents for the C₁₋₆alkyl and cyclopropyl are independently as described in the first set of variables of formula (I); and the other variables are each and independently as described above in any one of the first through twenty third sets of variables of formula (I).

In the thirty first set of variables of formula (I), R² is —(V²)—Y²; b is 0; Y² is a C₅₋₇ cycloalkyl group or 5-6 membered saturated, partially unsaturated, or aromatic monocyclic ring having 0-4 heteroatoms selected from oxygen, nitrogen, or sulfur, wherein Y² is optionally substituted with 1-4 occurrences of J^{Y}: R⁴ is optionally substituted C₁₋₆alkyl, optionally substituted cyclopropyl, or halogen, such as —F, or —Cl, wherein specific examples of substituents for the C₁₋₆alkyl and cyclopropyl are independently as described in the first set of variables of formula (I); and the other variables are each and independently as described above in any one of the first through twenty third sets of variables of formula (I).

In the thirty second set of variables of formula (I), R² is —(V²)_{b}—Y²; b is 0: Y² is phenyl optionally substituted with 1-4 occurrences of halogen: R⁴ is optionally substituted C₁₋₆alkyl, optionally substituted cyclopropyl, or halogen, such as —F, or —Cl, wherein specific examples of substituents for the C₁₋₆alkyl and cyclopropyl are independently as described in the first set of variables of formula (I); and the other variables are each and independently as described above in any one of the first through twenty third sets of variables of formula (I).

In the thirty third set of variables of formula (I), R² is —(V)_{b}—Y²; b is 0: Y² is a C₅₋₇ cycloalkyl group optionally substituted with 1-4 occurrences of halogen or C₁₋₄alkyl; R⁴ is optionally substituted C₁₋₆alkyl, optionally substituted cyclopropyl, or halogen, such as —F, or —Cl, wherein specific examples of substituents for the C₁₋₆alkyl and cyclopropyl are independently as described in the first set of variables of formula (I); and the other variables are each and independently as described above in any one of the first through twenty third sets of variables of formula (I).

In the thirty fourth set of variables of formula (I), R⁴ iso-propyl, tert-butyl, or cyclopropyl optionally substituted with one occurrence of CN or CH₃; and the other variables are each and independently as described above in any one of the first through the twenty third sets of variables of formula (I).

In the thirty fifth set of variables of formula (I), R² is —(V²)_{b}—Y²; b is 0; R⁴ iso-propyl, tert-butyl, or cyclopropyl optionally substituted with one occurrence of CN or CH₃; and the other variables are each and independently as described above in any one of the first through twenty third sets of variables of formula (I).

In the thirty sixth set of variables of formula (I), R² is —(V²)_{b}—Y²; b is 0; Y² is a C₅₋₇ cycloalkyl group or 5-6 membered saturated, partially unsaturated, or aromatic monocyclic ring having 0-4 heteroatoms selected from oxygen, nitrogen, or sulfur, wherein Y² is optionally substituted with 1-4 occurrences of J^{Y}: R⁴ iso-propyl, tert-butyl, or cyclopropyl optionally substituted with one occurrence of CN or CH₃; and the other variables are each and independently as described above in any one of the first through twenty third sets of variables of formula (I).

In the thirty seventh set of variables of formula (I), R² is —(V²)_{b}—Y; b is 0; Y² is phenyl optionally substituted with 1-4 occurrences of halogen; R⁴ iso-propyl, tert-butyl, or cyclopropyl optionally substituted with one occurrence of CN or CH₃; and the other variables are each and independently as described above in any one of the first through twenty third sets of variables of formula (I).

In the thirty eighth set of variables of formula (I), R² is —(V²)_{b}—Y²; b is 0; Y² is a C₅₋₇ cycloalkyl group optionally substituted with 1-4 occurrences of halogen or C₁₋₄alkyl; R⁴ iso-propyl, tert-butyl, or cyclopropyl optionally substituted with one occurrence of CN or CH₃; and the other variables are each and independently as described above in any one of the first through twenty third sets of variables of formula (I).

In the thirty ninth set of variables of formula (I), R² is —(V²)_{b}—Y²; b is 0; Y² is a non-aromatic, 5-7-membered, heterocycylic ring having 1-2 heteroatoms selected from oxygen, nitrogen, or sulfur, and optionally substituted with 1-4 occurrences of halogen or C₁₋₄alkyl; R⁴ is optionally substituted C₁₋₆alkyl, optionally substituted cyclopropyl, or halogen, such as —F, or —Cl, wherein specific examples of substituents for the C₁₋₆alkyl and cyclopropyl are independently as described in the first set of variables of formula (I); and the other variables are each and independently as described above in any one of the first through twenty third sets of variables of formula (I).

In the fourtieth set of variables of formula (I), R² is —(V²)_{b}—Y²; b is 0; Y² is a non-aromatic, 5-7-membered, heterocycylic ring having 1-2 heteroatoms selected from oxygen, nitrogen, or sulfur, and optionally substituted with 1-4 occurrences of halogen or C₁₋₄alkyl; R⁴ iso-propyl, tert-butyl, or cyclopropyl optionally substituted with one occurrence of CN or CH₃; and the other variables are each and independently as described above in any one of the first through twenty third sets of variables of formula (I).

In another embodiment, the invention is directed to compounds represented by formula (II) and pharmaceutically acceptable salts thereof:

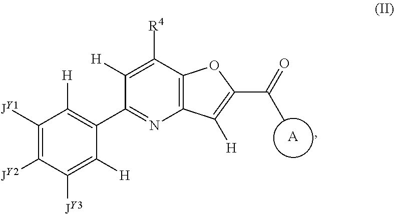

wherein the variables of formula (II) are each and independently as described herein.

In the first set of variables of formula (II), each of J^{Y}, J^{Y2}, and J^{Y3} is independently as defined for J^{Y} in the first set of variables formula (I); and J^{Y}, R⁴, and Ring A variables of formula (II) are each and independently as described above in the first set of variables formula (I).

In the second set of variables of formula (II), Ring A

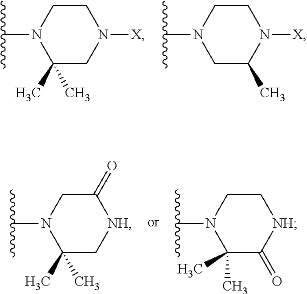

R⁴ is iso-propyl, tert-butyl, methylcyclopropyl, or cyanocyclopropyl;
X is R⁵ or —C(O)R⁵;
R⁵ is (V)ₐ—Y; wherein
V is C₁₋₆aliphatic wherein up to two carbon units of said C₁₋₆aliphatic can each be optionally and independently replaced with —O— or —C(O)—; wherein V is optionally substituted with 1-3 occurrences of halogen, CN. C₁₋₄alkyl, OH, O(C₁₋₄alkyl), NH₂, or —NHC(O)C₁₋₄alkyl;
Y is H, C₁₋₄ aliphatic, a 3-6 membered cycloalkyl, isoxazolyl, oxadiazolyl, pyrazolyl, imidazolyl, triazolyl, thienyl, pyrazolyl, tetrahydrofuranyl, tetrahydropyranyl, pyridinyl, pyrimidinyl, phenyl, oxetanyl, azetidinyl, pyrrolidinyl, piperidinyl, piperazinyl, thiomorpholinyl, morpholinyl, pyrazinyl, pyridazinyl, tetrazolyl, oxazolyl, thiazolyl, spiro[3,3]hepatanyl, or 1,1-dioxide-isothiazolidinyl; wherein Y is optionally substituted with 1-4 occurrences of J^{Y}; and
a is 0 or 1; and
   each J^{Y1}, J^{Y2}, and J^{Y3} is independently H, halogen, CN, C₁₋₄alkyl, haloC₁₋₄alkyl, or OH; and J^{Y} and any other variables are each independently as described in the first set of variables of formula (II).

In the third set of variables of formula (II), Ring A is

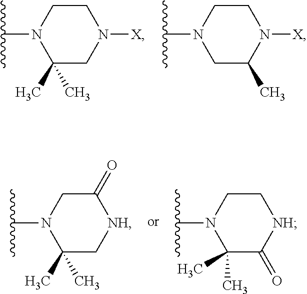

R is iso-propyl, tert-butyl, methylcyclopropyl, or cyanocyclopropyl;
X is R⁵ or —C(O)R⁵;
R⁵ is (V)ₐ—Y;
V is C₁₋₆aliphatic wherein up to two carbon units of said C₁₋₆aliphatic can each be optionally and independently replaced with —O— or —S(O)₂—; wherein V is optionally substituted with 1-3 occurrences of halogen, CN, C₁₋₄alkyl, OH, O(C₁₋₄ alkyl), NH₂, or —NHC(O)C₁₋₄alkyl;
Y is H, C₁₋₄aliphatic, a 3-6 membered cycloalkyl, isoxazolyl, oxadiazolyl, pyrazolyl, imidazolyl, triazolyl, thienyl, pyrazolyl, tetrahydrofuranyl, tetrahydropyranyl, pyridyl, pyrimidyl, or phenyl: wherein Y is optionally substituted with 1-4 occurrences of J^{Y}; and
a is 0 or 1; and
each J^{Y1}, J^{Y2}, and J^{Y3} is independently H, halogen, CN, C₁₋₄alkyl, haloC₁₋₄alkyl, or OH; and J^{Y} and any other variables are each independently as described in the first set of variables of formula (II).

Alternatively, V is C₁₋₆aliphatic wherein up to two carbon units of said C₁₋₆aliphatic can each be optionally and independently replaced with —O— or —C(O)—; wherein V is optionally substituted with 1-3 occurrences of halogen, CN, C₁₋₄alkyl, OH, O(C₁₋₄alkyl), NH₂, or —NHC(O)C₁₋₄alkyl; and Y is H, a 3-6 membered cycloalkyl, isoxazolyl, oxadiazolyl, pyrazolyl, imidazolyl, triazolyl, thienyl, pyrazolyl, tetrahydrofuranyl, tetrahydropyranyl, pyridyl, pyrimidyl, or phenyl; wherein Y is optionally substituted with 1-4 occurrences of J^{Y}.

In the fourth set of variables of formula (II), Ring A is as described in any one of the first through third sets of variables of formula (II); R⁴ is iso-propyl, tert-butyl, methylcyclopropyl, or cyanocyclopropyl; each J^{Y1}, J^{Y2}, and J^{Y3} is independently H, halogen, CN, C₁₋₄alkyl, haloC₁₋₄alkyl, or OH: X is selected from the group consisting of —C(O)CH(CH₃)OH, —C(O)OC(CH₃)₃, —C(O)C(CH₃)₂OH, —C(O)C(OH)(cyclobutyl), —C(O)CH₂CN, —C(O)tetrahydrofuranyl, —C(O)phenyl, —C(O)isoxazolyl, —C(O)CH(OH)CH(CH₃)₂, —C(O)CH(CH₃)₃, —C(O)CH(OH)CH₃, —C(O)C(CH₃)₂F, —C(O)CH₂OCH₃, —C(O)CH(OH)CH₂C(CH₃)₃, —C(O)methylcyclopropyl, —C(O)dimethylcyclopropyl, —C(O)gem dimethylcyclopropyl. —C(O)C═CCH₃, —C(O)CH₂C(CH₃)₃, —C(O)hydroxyl-tetrahydropyranyl, —C(O)CH₃, —C(O)CH(OH)CH₂CH₃, —C(O)CH(OCH₃)CH₃, —C(O)tetrahydrofuranyl, —C(O)CH(OH)(cyclopropyl),

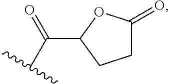 [SMILES: *C(=O)C1CCC(=O)O1]

C(O)CH(OH)(CH₂)₃CH₃, —C(O)C(CH₃)(CH₂CH₃)OH, —C(O)CH(OH)CH₂CH(CH₃)₂, —C(O)CH₂OCH₂CH₂OCH₃, —C(O)CH(OH)CH₂(CH₃)₃, —C(O)C(CH₃)(CF₃)OH, —CH(OH)(cyclohexyl), —CH(OH)CH₂phenyl, —C(O)cyclohexyl, —C(O)(methylcyclohexyl), —C(O)CH(CH₃)CH₂CH₃, —C(O)CH(phenyl)CH₂CH₃, —C(O)cyclobutyl, —C(O)cyclopropyl), —C(O)(tetramethylcyclopropyl), —C(O)(cyanocyclopropyl), —C(O)C(CH₃)₂CH₂CH₃,

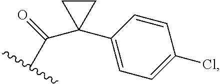 [SMILES: *C(=O)C1(c2ccc(Cl)cc2)CC1]

C(O)(tetrahydropyranyl), —C(O)(CF₃-cyclopropyl), —C(O)(hydroxycyclobutyl), —C(O)(hydroxytetrahydropyranyl),

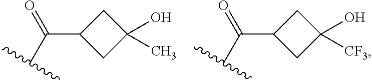

C(O)CH₂C(CH₃)₂OH, —C(O)pyrazolyl, —C(O)CH(OCH₃)(phenyl), —C(O)CH(CH₃)(phenyl), —C(O)C(CF₃)(OH)CH₃, —C(O)(OH)CH₂CH₃)₂,

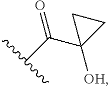 [SMILES: *C(=O)C1(O)CC1]

—C(O)cyclopentyl, —C(O)(difluorocyclopropyl), —C(O)isoxazolyl, —C(O)C(CH₃)₃, —C(O)CH(OH)(4-fluorphenyl), —C(O)OCH₂CH₃, —C(CH₃)₂CH₂OCH₃, and —C(O)dimethylpyrazolyl; and any other variables are each independently as described in the first set of variables of formula (II)

In the fifth set of variables of formula (II), Ring A, R⁴, J^{Y1}, J^{Y2}, and J^{Y3} are each independently as described in any one of the first through third sets of variables of formula (II); and X is selected from the group consisting of:

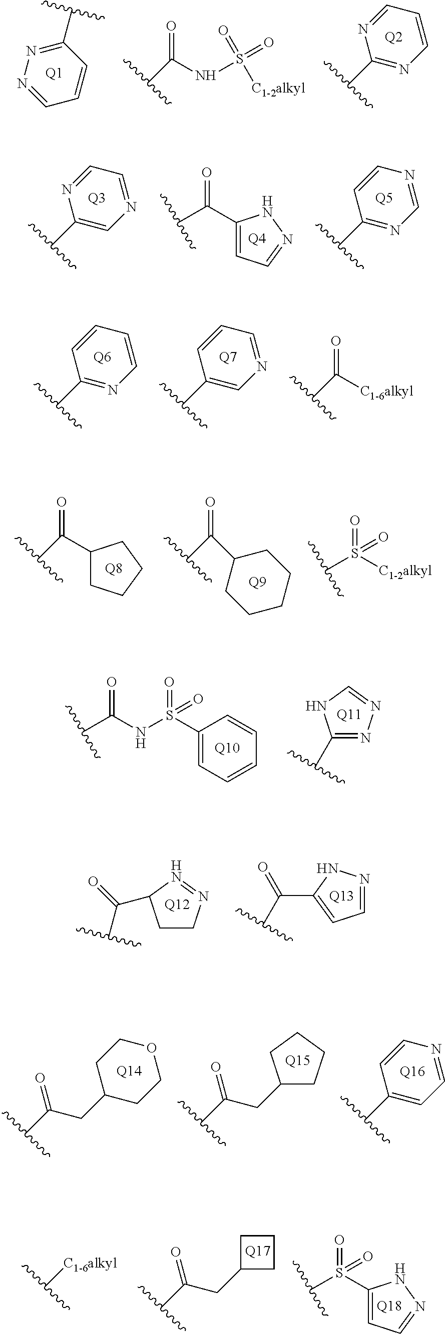

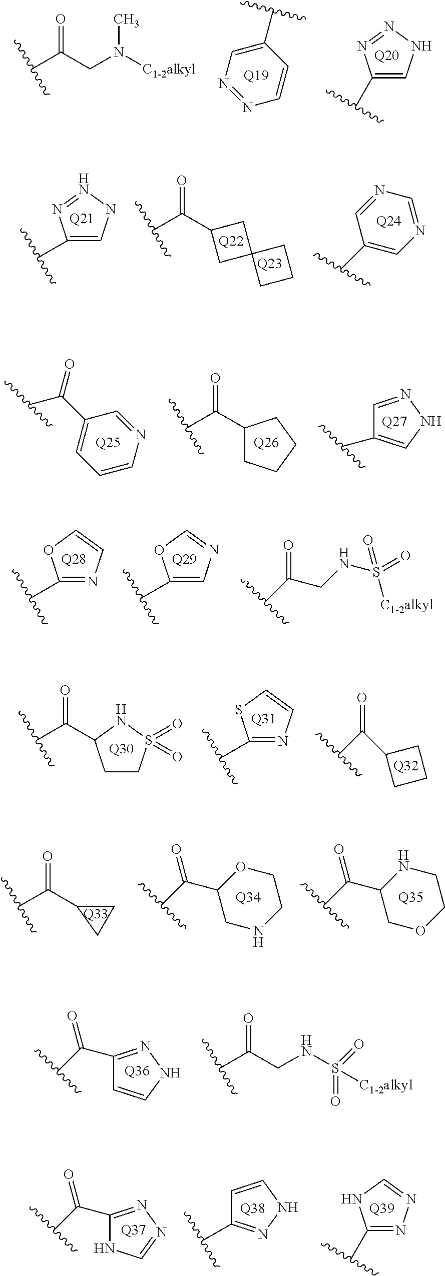

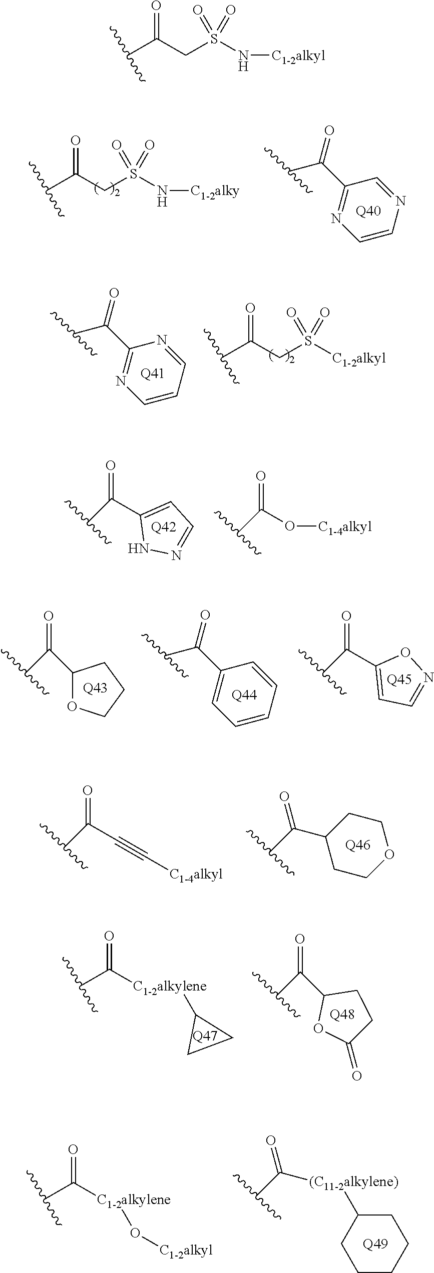

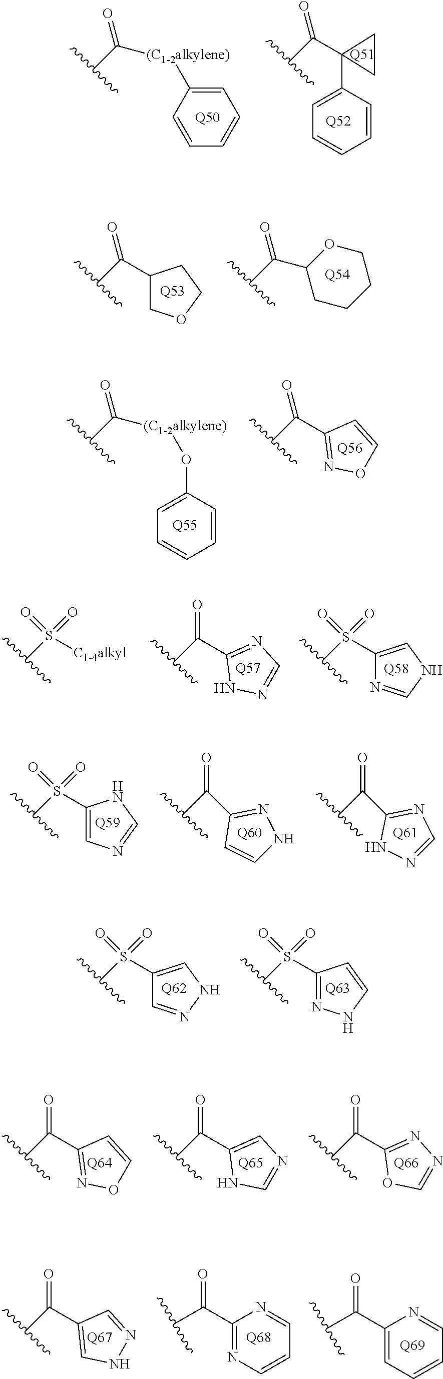

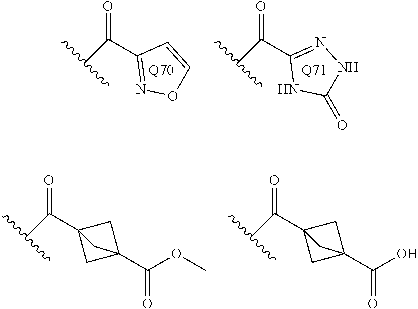

each of Rings Q1-Q71 is optionally and independently substituted with 1-4 occurrences of J^{Y}, wherein J^{Y} is each independently selected from —CN, halogen, —CH₃, —CF₃, —OH, —OCH₃, —NH₂, —NHCH₃, N(CH₃)₂, —C(O)OH, or —C(O)O(C₁₋₂alkyl);

each of said C₁₋₂alkyl, C₁₋₂alkylene, C₁₋₄alkyl, and C₁₋₆alkyl of X is optionally and independently substituted with 1-4 occurrences of J^{V}, where J^{V} is each independently selected from —CN, halogen, —CH₃, —CF₃, —OH, —OCH₃, —NH₂, —NHCH₃, N(CH₃)₂, —C(O)OH, or —C(O)O(C₁₋₂ alkyl); and

any other variables are each independently as described in the first set of variables of formula (II).

In the sixth set of variables of formula (II). Ring A is as described in any one of the first through third sets of variables of formula (II); R⁴ is iso-propyl, tert-butyl, methylcyclopropyl, or cyanocyclopropyl; each J^{Y1}, J^{Y2} and J^{Y3} is independently H, halogen, CN, C₁₋₄alkyl, haloC₁₋₄alkyl, or OH; and X is selected from the group consisting of:

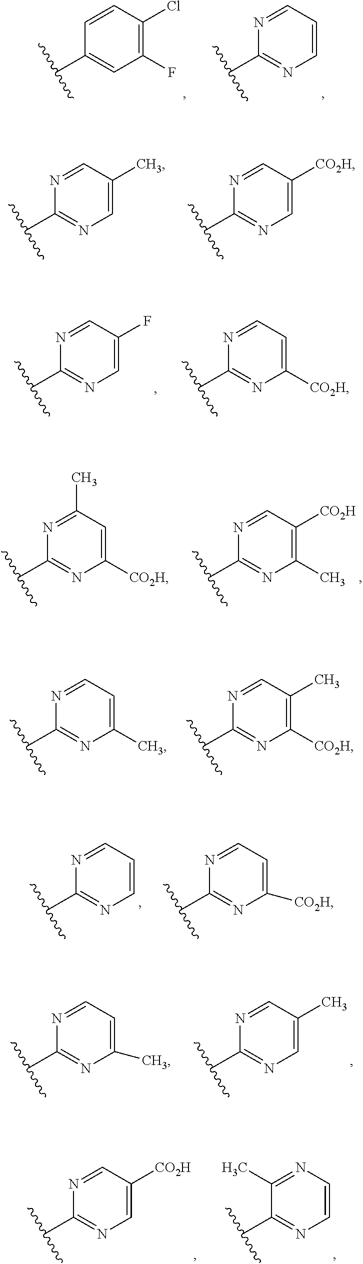

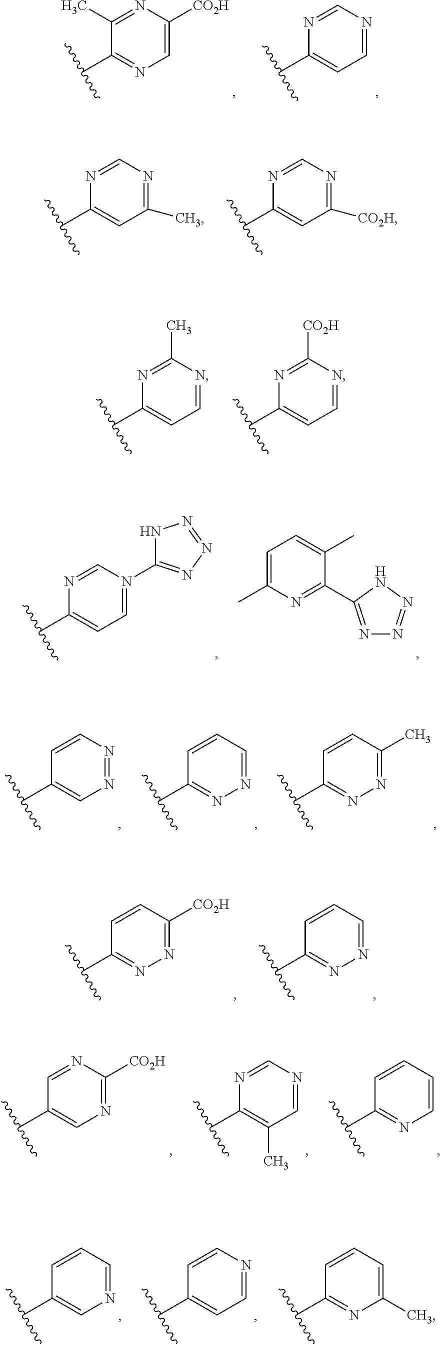

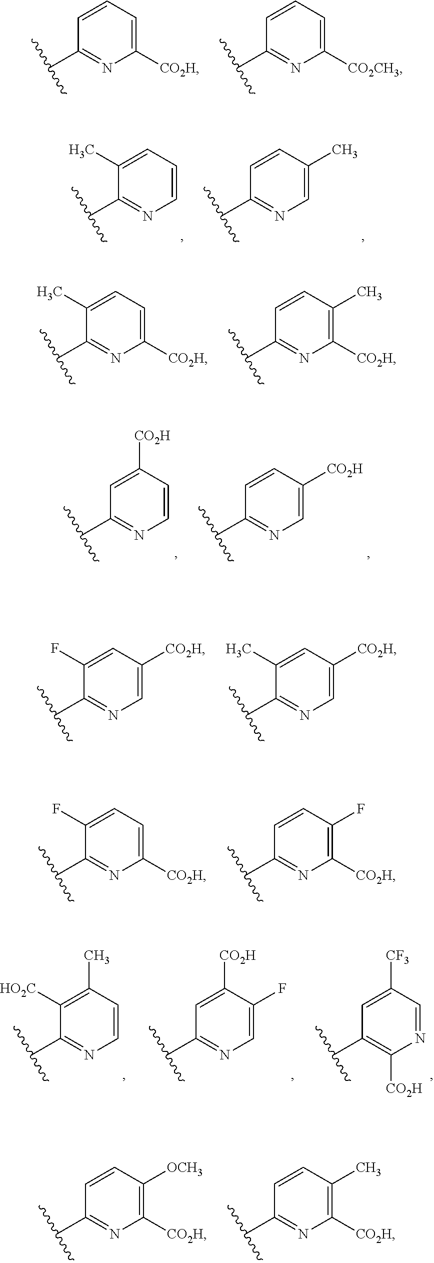

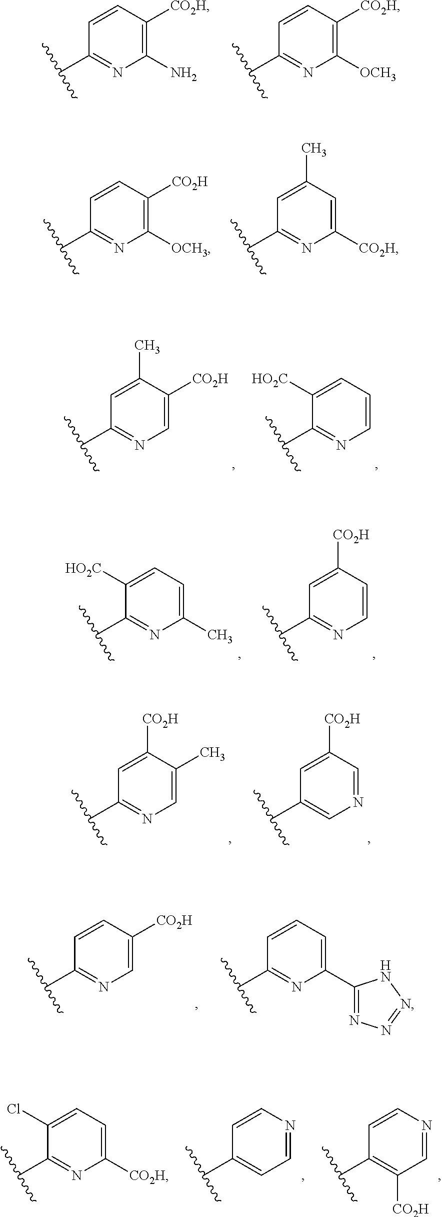

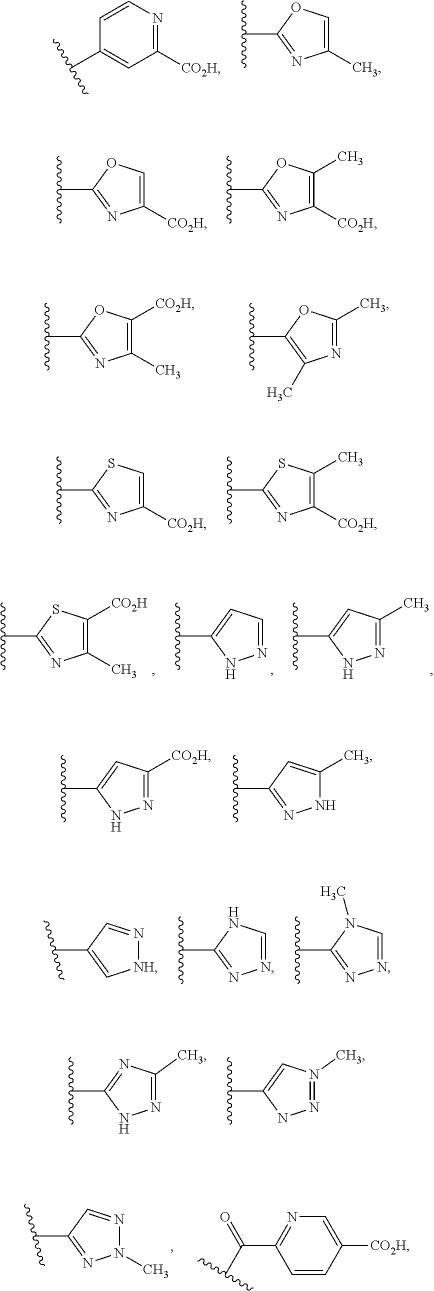

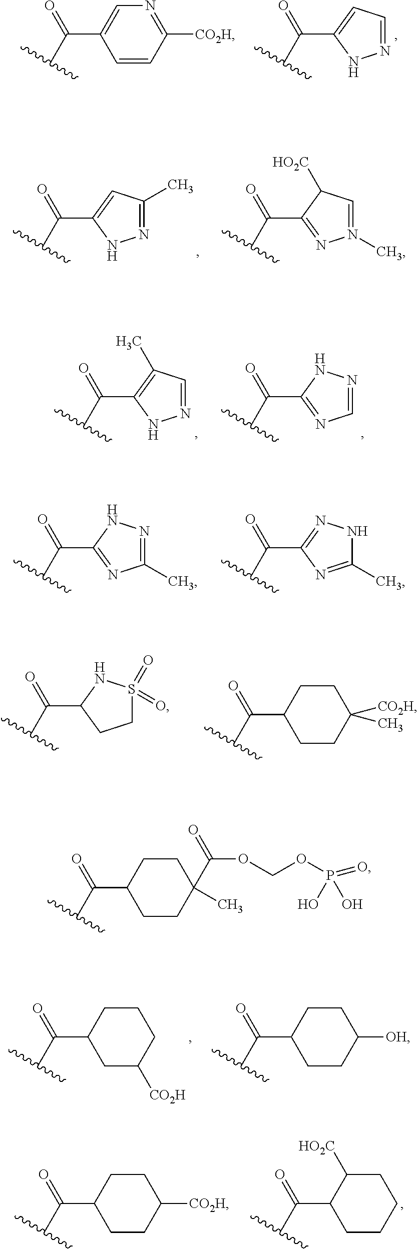

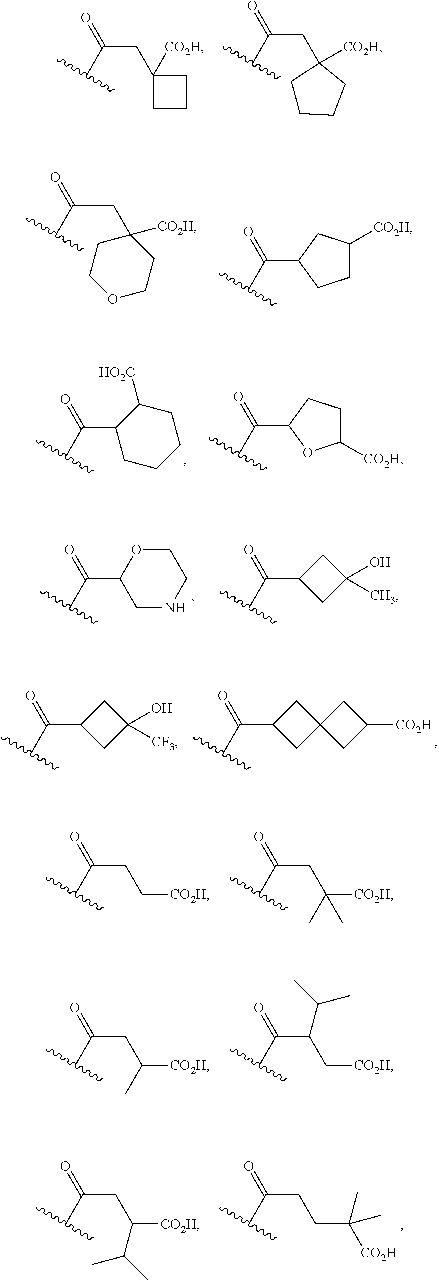

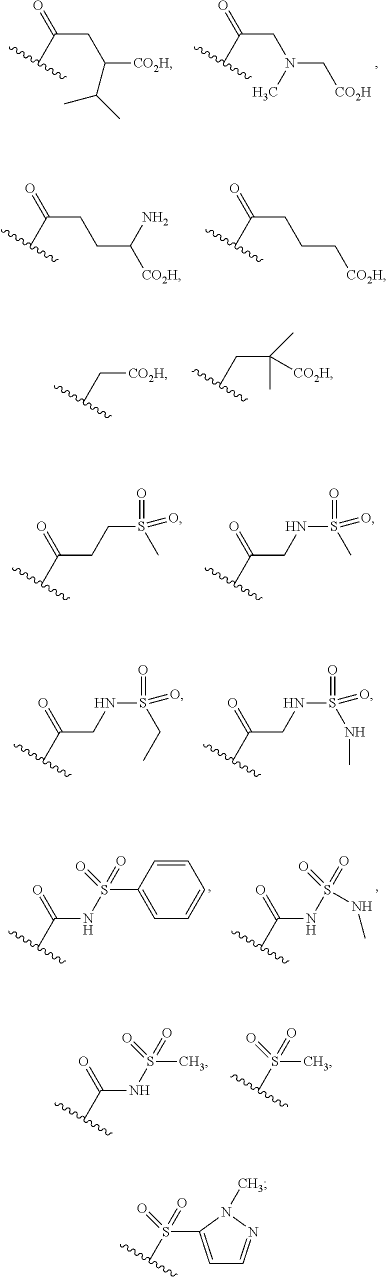

any other variables are each independently as described in the first set of variables of formula (II).

In the seventh set of variables of formula (II), Ring A is as described in any one of the first through third sets of variables of formula (II): R⁴ is iso-propyl, tert-butyl, methylcyclopropyl, or cyanocyclopropyl: each J^{Y1}, J^{Y2}, and J^{Y3} is independently H, halogen, CN, C₁₋₄alkyl, haloC₁₋₄alkyl, or OH; X is selected from the group consisting of:

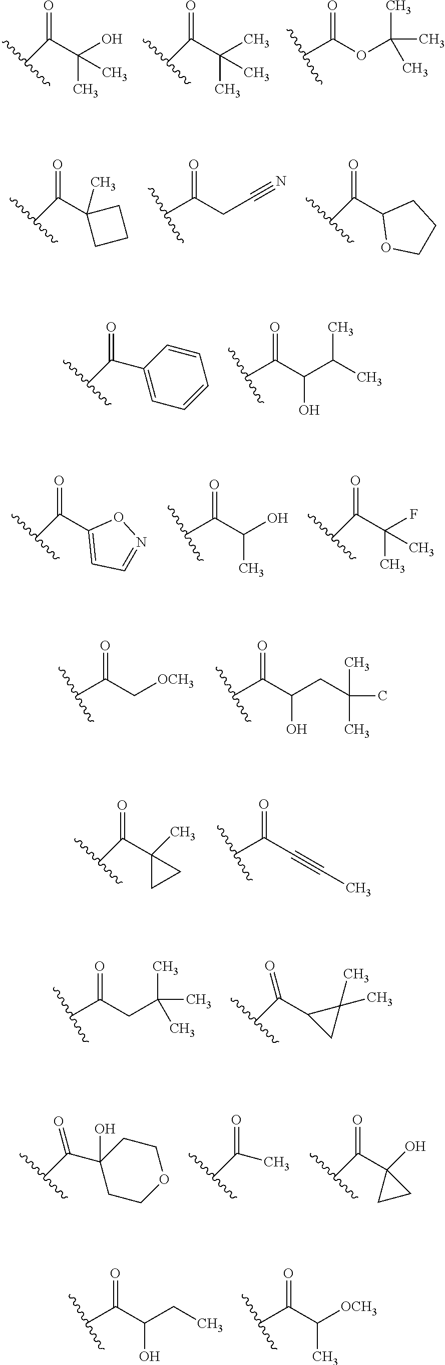

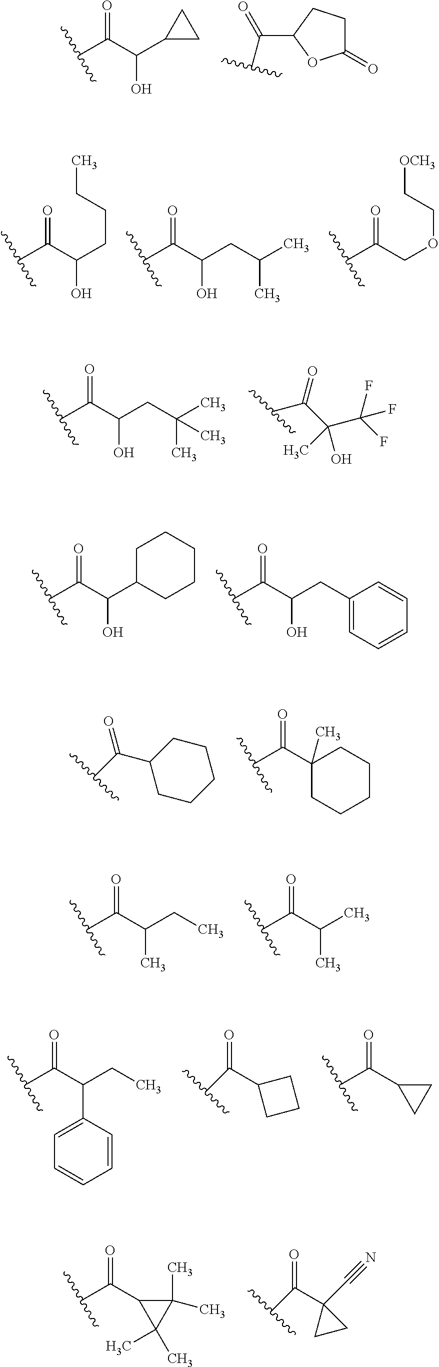

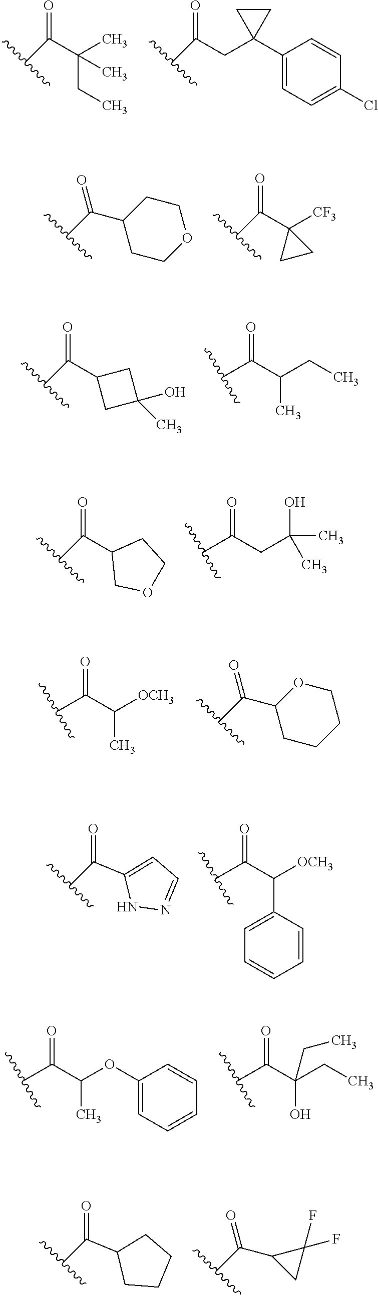

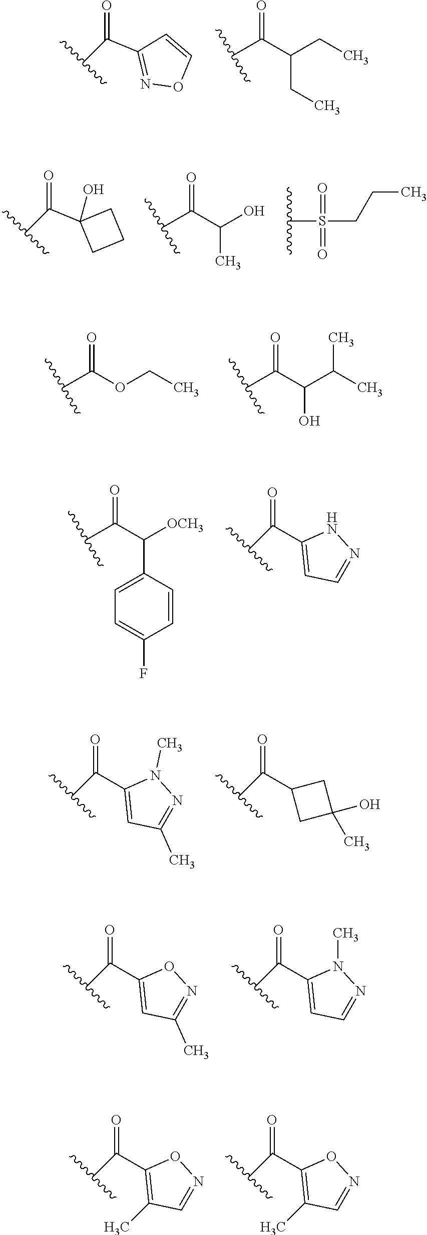

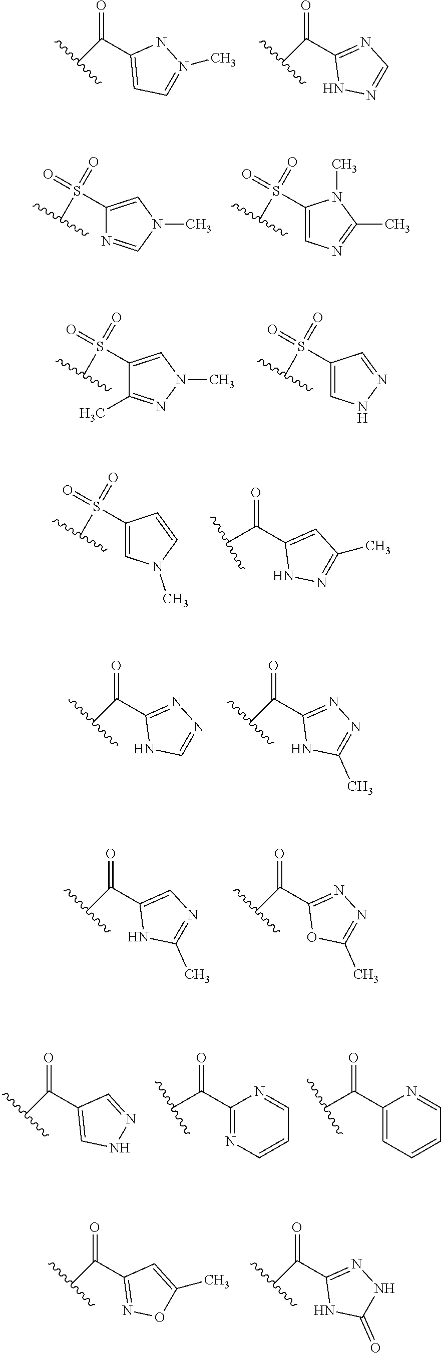

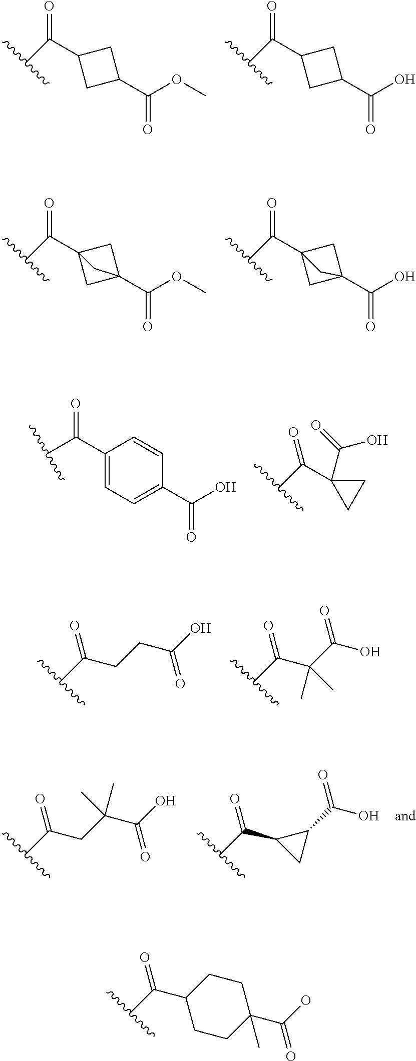

and other variables are each independently as described in the first set of variables of formula (II).

In the ninth set of variables of formula (II), each of J^{Y1}, J^{Y2} and J^{Y3} is independently H, Cl, F, CH₃, or CF₃; and the other variables are each and independently as described in the first through eighth sets of variables of formula (II)

In the tenth set of variables of formula (II), Ring A is

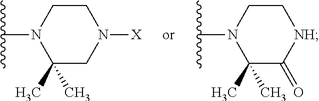

R⁴ is iso-propyl, tert-butyl, methylcyclopropyl, or C(CH₃)₂CH₂OCH₃;

### X is

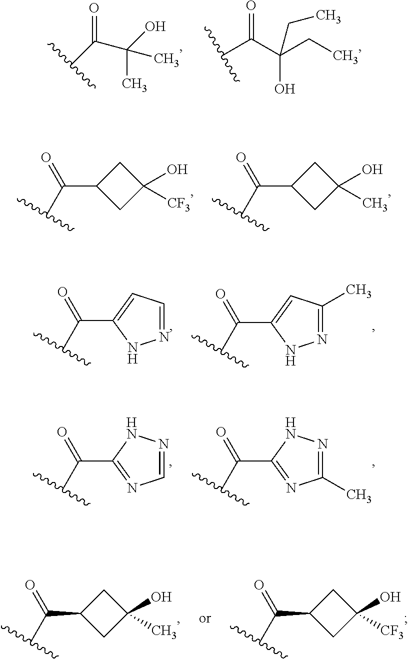

and each J^{Y1}, J^{Y2}, and J^{Y3} is independently H, halogen, C₁₋₄alkyl, or haloC₁₋₄alkyl. The other variables are each and independently as described in the first set of variables of formula (II).

In the eleventh set of variables of formula (II), Ring A is

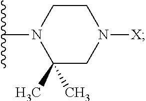

and the other variables are each and independently as described in the first set of variables of formula (II).

In the twelfth set of variables of formula (II), Ring A is

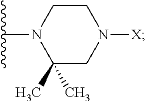

R⁴ is iso-propyl, tert-butyl, methylcyclopropyl, or C(CH₃)₂CH₂OCH₃;

### X is

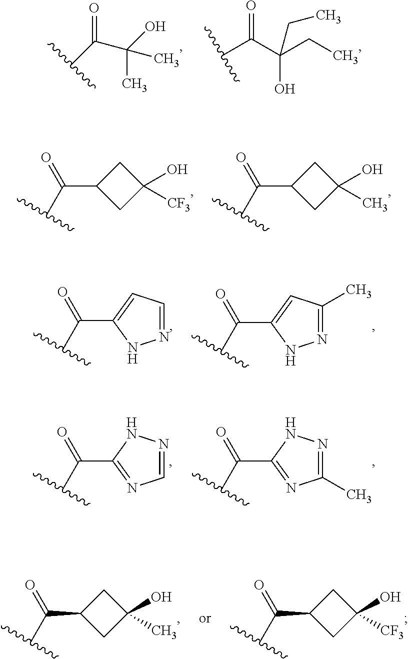

each J^{Y1}, J^{Y2}, and J^{Y3} is independently H, halogen. C₁₋₄alkyl, or haloC₁₋₄alkyl; and the other variables are each and independently as described in the first set of variables of formula (II).

In the thirteenth set of variables of formula (II). J^{Y1} is H, F, or CH₃; J^{Y3} is H; J^{Y2} is Cl, F, CH₃, or CF₃; and the other variables are each and independently as described in any one of the first through the twelfth sets of variables of formula (II).

In the fourteenth set of variables of formula (II). Ring A is

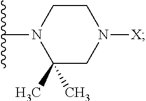

R⁴ is iso-propyl, tert-butyl, methylcyclopropyl, or C(CH₃)₂CH₂OCH₃;

### X is

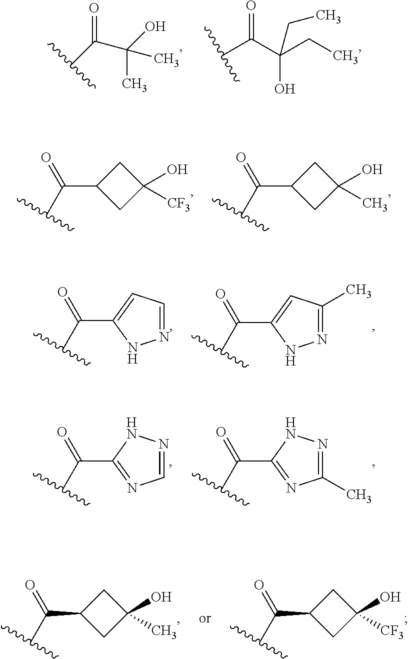

J^{Y1} is H, F, or CH₃; J^{Y3} is H; J^{Y2} is Cl, F, CH₃, or CF₃; and the other variables are each and independently as described in the first set of variables of formula (II).

In the fifteenth set of variables of formula (II), Ring A is

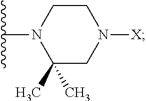

### X is

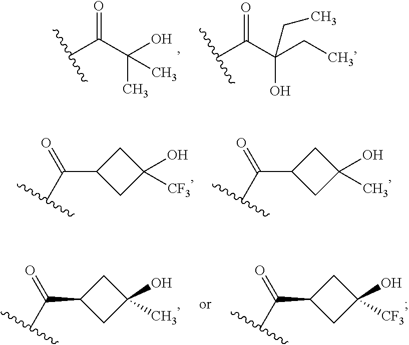

and the other variables are each and independently as described in the first set of variables of formula (II).

In the sixteenth set of variables of formula (II), Ring A is

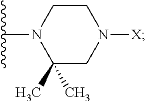

R⁴ is iso-propyl, tert-butyl, methylcyclopropyl, or C(CH₃)₂CH₂OCH₃; X is

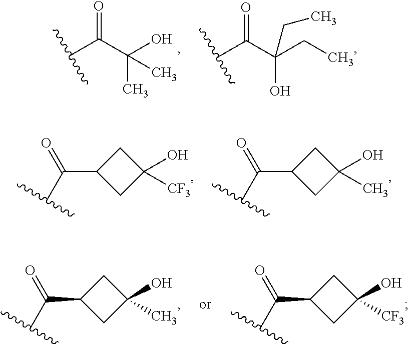

each J^{Y1}, J^{Y2} and J^{Y3} is independently H, halogen, C₁₋₄alkyl, or haloC₁₋₄alkyl; and the other variables are each and independently as described in the first set of variables of formula (II).

In the seventeenth set of variables of formula (II), Ring A is

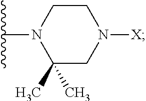

R⁴ is iso-propyl, tert-butyl, methylcyclopropyl, or C(CH₃)₂CH₂OCH₃; X is

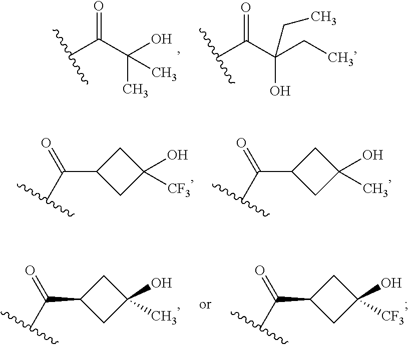

J^{Y1} is H, F, or CH₃; J^{Y3} is H; J^{Y2} is Cl, F, CH₃, or CF₃; and the other variables are each and independently as described in the first set of variables of formula (II).

In the eighteenth set of variables of formula (II), Ring A is

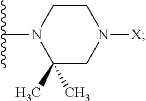

### X is

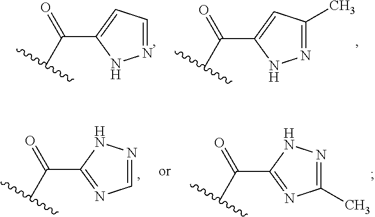

and the other variables are each and independently as described in the first set of variables of formula (II).

In the nineteenth set of variables of formula (II), Ring A is

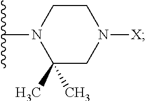

R⁴ is iso-propyl, tert-butyl, methylcyclopropyl, or C(CH₃)₂CH₂OCH₃; X is

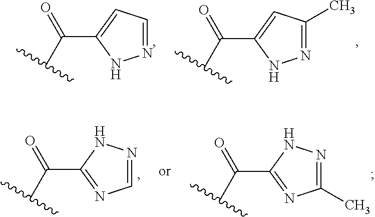

each J^{Y1}, J^{Y2}, and J^{Y3} is independently H, halogen, C₁₋₄alkyl, or haloC₁₋₄alkyl and the other variables are each and independently as described in the first set of variables of formula (II).

In the twentieth set of variables of formula (II), Ring A is

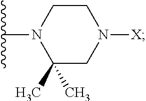

R⁴ is iso-propyl, tert-butyl, methylcyclopropyl, or C(CH₃)₂CH₂OCH; X is

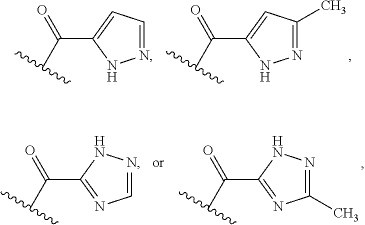

J^{Y1} is H, F, or CH₃; J^{Y3} is H; J^{Y2} is Cl, F, CH₃, or CF₃; and the other variables are each and independently as described in the first set of variables of formula (II).

In the twenty first set of variables of formula (II), Ring A is

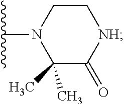

and the other variables are each and independently as described in the first set of variables of formula (II).

In the twenty second set of variables of formula (II), Ring A is

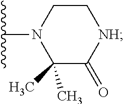

R⁴ is iso-propyl, tert-butyl, methylcyclopropyl, or C(CH₃)₂CH₂OCH₃; each J^{Y1}, J^{Y2}, and J^{Y3} is independently H, halogen, C₁₋₄alkyl, or haloC₁₋₄alkyl; and the other variables are each and independently as described in the first set of variables of formula (II).

In the twenty third set of variables of formula (II), Ring A is

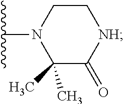

R⁴ is iso-propyl, tert-butyl, methylcyclopropyl, or C(CH₃)₂CH₂OCH₃; J^{Y1} is H, F, or CH₃; J^{Y3} is H; J^{Y2} is Cl, F, CH₃, or CF₃; and the other variables are each and independently as described in the first set of variables of formula (II).

In the twenty fourth set of variables of formula (II), Ring A is

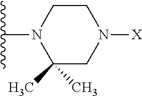

R⁴ is iso-propyl, tert-butyl, methylcyclopropyl, or C(CH₃)₂CH₂OCH₃;

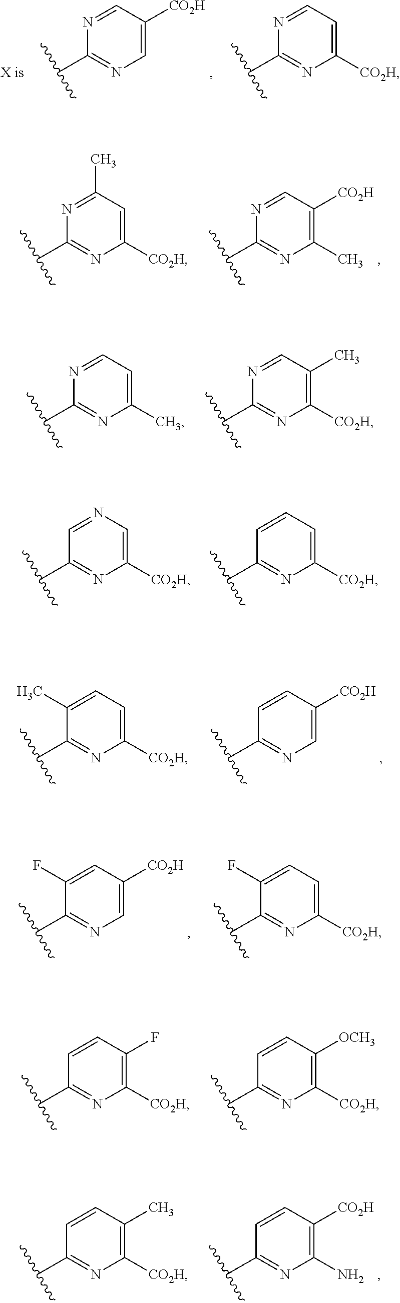

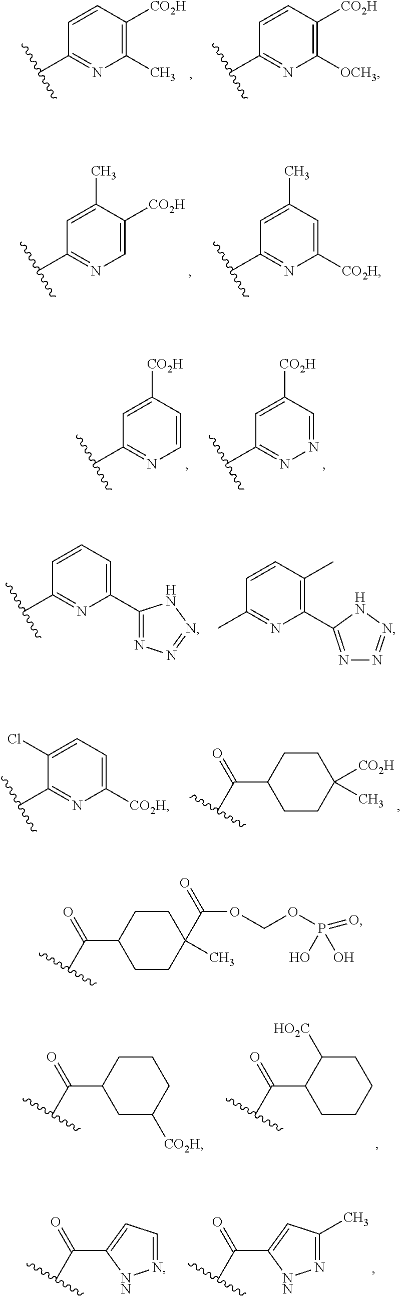

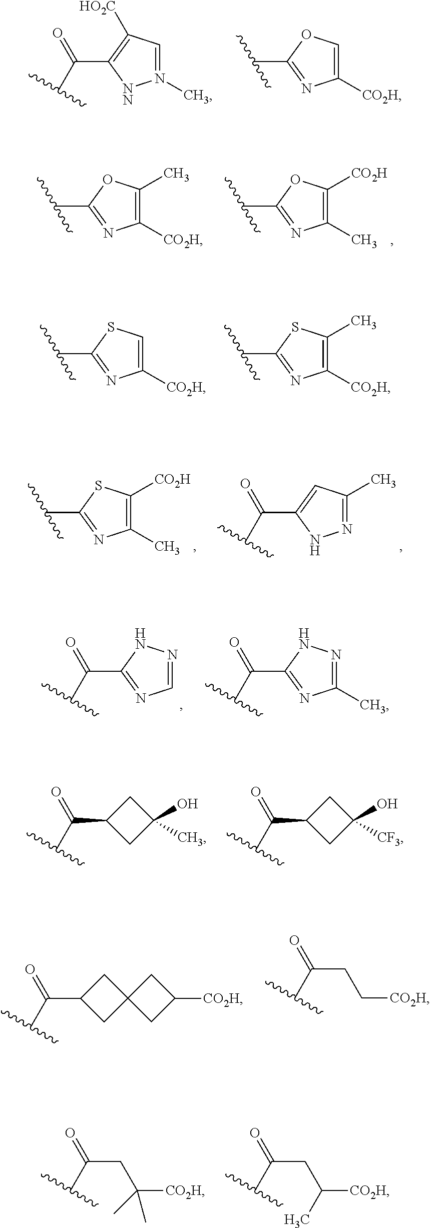

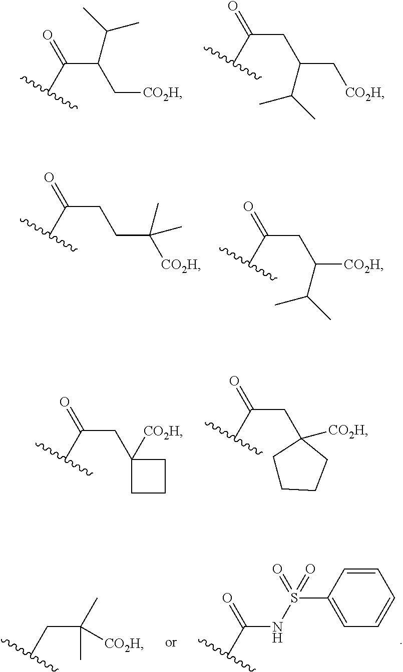

and

each J^{Y1}, J^{Y2}, and J^{Y3} is independently H, halogen, C₁₋₄alkyl, or haloC₁₋₄alkyl;

and the other variables of formula (II) are independently as described in the first set of variables of formula (II).

In the twenty fifth set of variables of formula (II), Ring A is

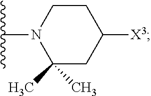

X³ is —CR₂—C(O)OR⁶, —N(R)R⁶, —CR₂—C(O)N(R)R⁶ or —CR₂—C(O)N(R)S(O)₂R⁶; each R⁶ is independently —H, C₁₋₆ alkyl optionally substituted with 1-4 occurrences of substituents selected from CN, halogen, C₁₋₄alkyl, haloC₁₋₄alkyl, —OH, —O(C₁₋₄alkyl). NH(C₁₋₄alkyl), N(C₁₋₄alkyl)₂, S(O)₂(C₁₋₄alkyl), NH₂, (CH₂)ₜ—C(O)OH, (CH₂)ₜ—C(O)O(C₁₋₂ alkyl), (CH(C₁₋₂ alkyl))ₜ—C(O)OH, (CH(C₁₋₂ alkyl))ₜ—C(O)O(C₁₋₂ alkyl), (C(C₁₋₂ alkyl)₂)ₜ—C(O)OH, or (C(C₁₋₂ alkyl)₂)ₜ—C(O)O(C₁₋₂ alkyl), wherein each t is independently 0 or 1; and the other variables of formula (II) are independently as described in the first set of variables of formula (II).

In the twenty sixth set of variables of formula (II), Ring A is

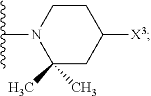

R⁴ is iso-propyl, tert-butyl, methylcyclopropyl, or C(CH₃)₂CH₂OCH₃; X³ is —CH₂—C(O)OH, —NH₂, —NH(C₁₋₂ alkyl), —CH₂—C(O)NH—C₁₋₂ alkyl, —CH₂—C(O)NHS(O)₂C₁₋₂ alkyl, or —CH₂—C(O)NH—CN, wherein each of said C₁₋₂ alkyl is optionally and independently substituted with 1-4 substitutents selected from CN, halogen, —OH, —OCH₃, or —C(O)OH; each J^{Y1}, J^{Y2}, and J^{Y3} is independently H, halogen, C₁₋₄alkyl, or haloC₁₋₄alkyl; and the other variables of formula (II) are independently as described in the first set of variables of formula (II).

In yet another embodiment, the invention is directed to compounds represented by formula (III) and pharmaceutically acceptable salts thereof:

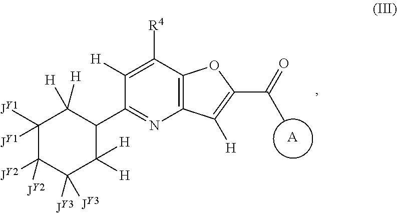

wherein the variables of formula (III) are each and independently as described herein.

In the first set of variables of formula (III), each of J^{Y1}, J^{Y2}, and J^{Y3} is independently as defined for J^{Y}; and J^{Y}, R⁴, and Ring A variables of formula (III) are each and independently as described above in the first set of variables formula (I).

The second set to eighth set of variables of formula (III) are each and independently as described in any one of the sets of variables for formula (II).

In the third set of variables of formula (III), Ring A is

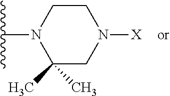

R⁴ is iso-propyl, tert-butyl, methylcyclopropyl, or C(CH₃)₂CH₂OCH₃;

### X is

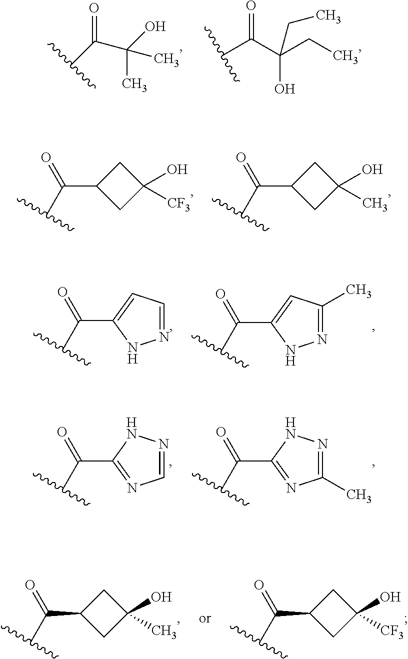

and each J^{Y1}, J^{Y2}, and J^{Y3} is independently H, halogen. C₁₋₄alkyl, or haloC₁₋₄alkyl. The other variables are each and independently as described above in the first set of variables of formula (III).

In the fourth set of variables of formula (III), Ring A is

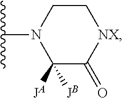

R⁴ is iso-propyl, tert-butyl, methylcyclopropyl, or C(CH₃)₂CH₂OCH₃;

### X is

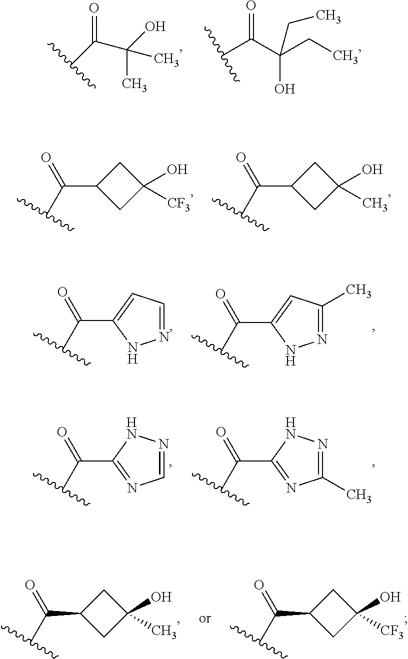

each of J^{Y1}, J^{Y2} and J^{Y3} is independently H, Cl, F, CH₃, or CF₃. The other variables are each and independently as described above in the first set of variables of formula (III).

In the fifth set of variables of formula (III), Ring A is

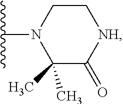

R⁴ is iso-propyl, tert-butyl, methylcyclopropyl, or C(CH₃)₂CH₂OCH₃; each J^{Y1}, J^{Y2}, and J^{Y3} is independently H, halogen, C₁₋₄alkyl, or haloC₁₋₄alkyl; and the other variables are each and independently as described above in the first set of variables of formula (III).

In the sixth set of variables of formula (III), Ring A is

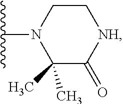

R⁴ is iso-propyl, tert-butyl, methylcyclopropyl, or C(CH₃)₂CH₂OCH₃; each of J^{Y1}, J^{Y2} and J^{Y3} is independently H, Cl, F, CH₃, or CF₃; and the other variables are each and independently as described above in the first set of variables of formula (III).

In yet another embodiment, the invention is directed to compounds represented by formula (II) and pharmaceutically acceptable salts thereof, wherein the variables are each and independently as described in any one of the sets of variables of formula (I).

In yet another embodiment, the invention is directed to compounds represented by formula (III) and pharmaceutically acceptable salts thereof, wherein the variables are each and independently as described in any one of the sets of variables of formula (I).

In another aspect, the invention is directed to compounds represented by any one of the following structural formulae and pharmaceutically acceptable salts thereof:

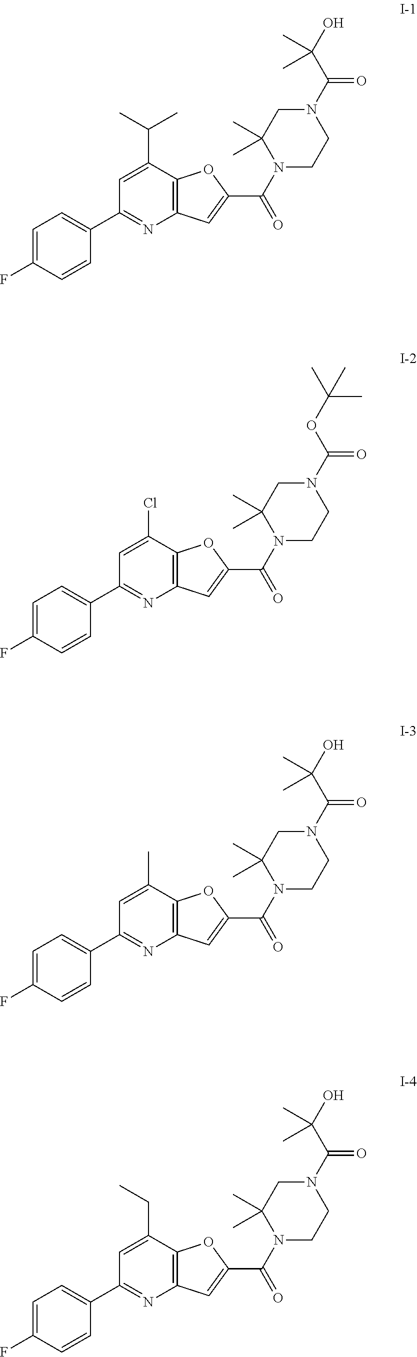 [SMILES: CC(C)c1cc(-c2ccc(F)cc2)nc2cc(C(=O)N3CCN(C(=O)C(C)(C)O)CC3(C)C)oc12 ; CC(C)(C)OC(=O)N1CCN(C(=O)c2cc3nc(-c4ccc(F)cc4)cc(Cl)c3o2)C(C)(C)C1 ; Cc1cc(-c2ccc(F)cc2)nc2cc(C(=O)N3CCN(C(=O)C(C)(C)O)CC3(C)C)oc12 ; CCc1cc(-c2ccc(F)cc2)nc2cc(C(=O)N3CCN(C(=O)C(C)(C)O)CC3(C)C)oc12]

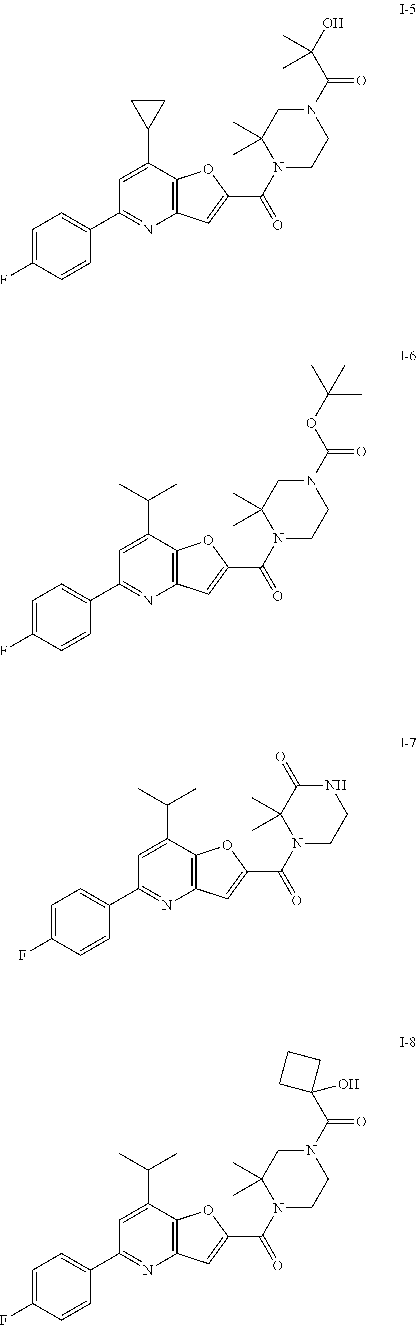 [SMILES: CC(C)(O)C(=O)N1CCN(C(=O)c2cc3nc(-c4ccc(F)cc4)cc(C4CC4)c3o2)C(C)(C)C1 ; CC(C)c1cc(-c2ccc(F)cc2)nc2cc(C(=O)N3CCN(C(=O)OC(C)(C)C)CC3(C)C)oc12 ; CC(C)c1cc(-c2ccc(F)cc2)nc2cc(C(=O)N3CCNC(=O)C3(C)C)oc12 ; CC(C)c1cc(-c2ccc(F)cc2)nc2cc(C(=O)N3CCN(C(=O)C4(O)CCC4)CC3(C)C)oc12]

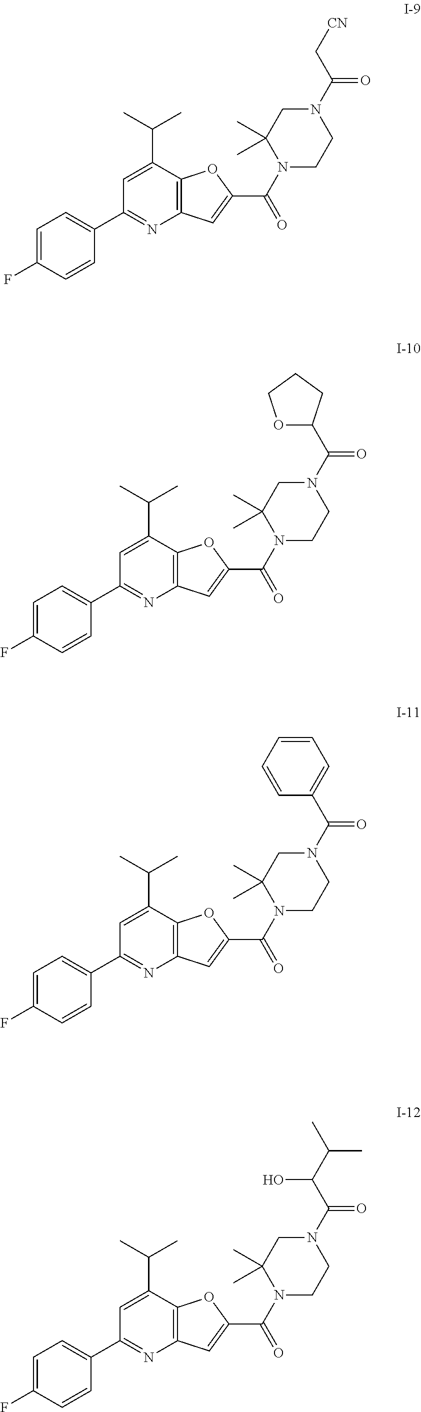 [SMILES: CC(C)c1cc(-c2ccc(F)cc2)nc2cc(C(=O)N3CCN(C(=O)CC#N)CC3(C)C)oc12 ; CC(C)c1cc(-c2ccc(F)cc2)nc2cc(C(=O)N3CCN(C(=O)C4CCCO4)CC3(C)C)oc12 ; CC(C)c1cc(-c2ccc(F)cc2)nc2cc(C(=O)N3CCN(C(=O)c4ccccc4)CC3(C)C)oc12 ; CC(C)c1cc(-c2ccc(F)cc2)nc2cc(C(=O)N3CCN(C(=O)C(O)C(C)C)CC3(C)C)oc12]

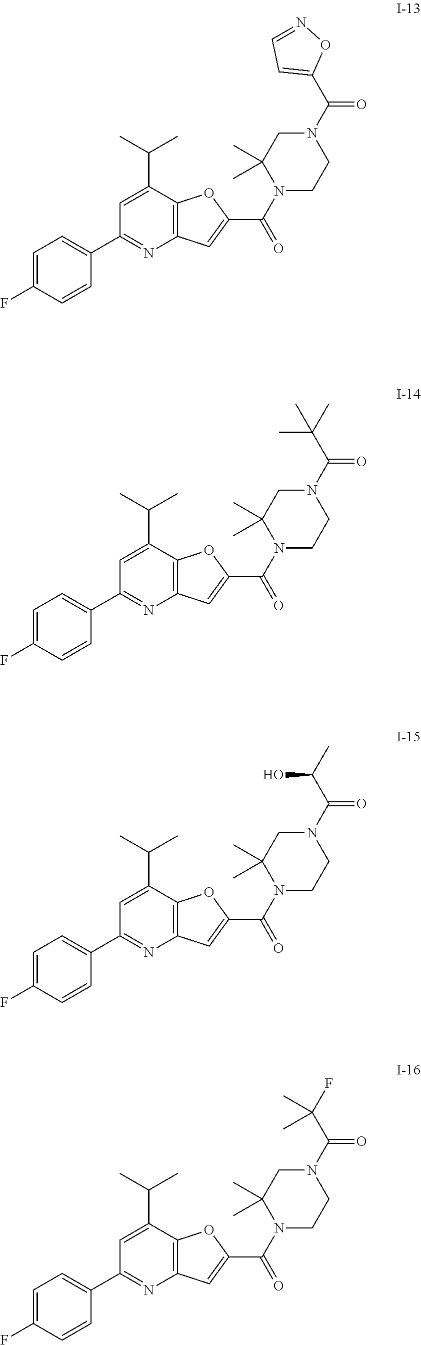 [SMILES: CC(C)c1cc(-c2ccc(F)cc2)nc2cc(C(=O)N3CCN(C(=O)c4ccno4)CC3(C)C)oc12 ; CC(C)c1cc(-c2ccc(F)cc2)nc2cc(C(=O)N3CCN(C(=O)C(C)(C)C)CC3(C)C)oc12 ; CC(C)c1cc(-c2ccc(F)cc2)nc2cc(C(=O)N3CCN(C(=O)[C@H](C)O)CC3(C)C)oc12 ; CC(C)c1cc(-c2ccc(F)cc2)nc2cc(C(=O)N3CCN(C(=O)C(C)(C)F)CC3(C)C)oc12]

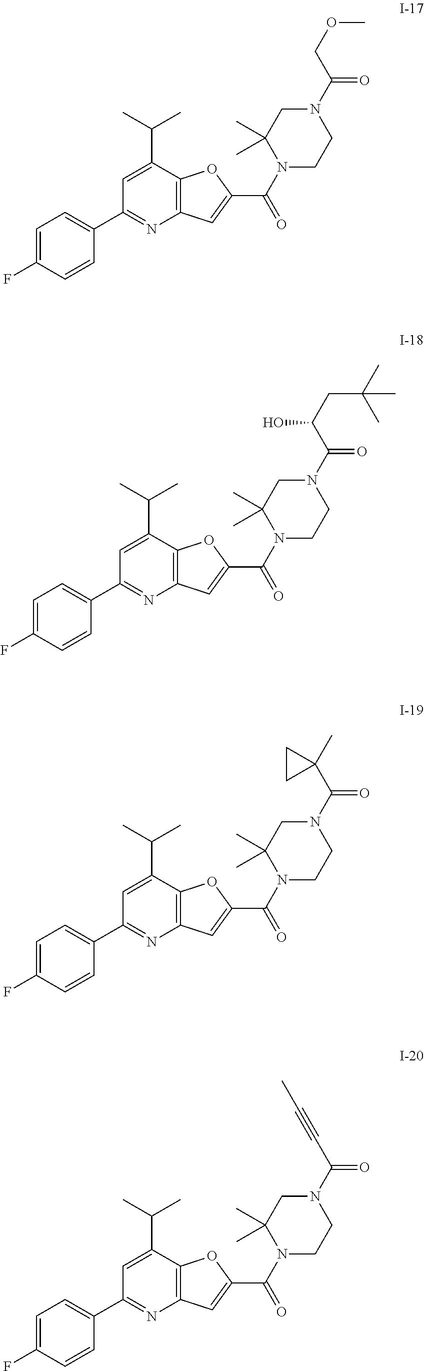 [SMILES: COCC(=O)N1CCN(C(=O)c2cc3nc(-c4ccc(F)cc4)cc(C(C)C)c3o2)C(C)(C)C1 ; CC(C)c1cc(-c2ccc(F)cc2)nc2cc(C(=O)N3CCN(C(=O)[C@H](O)CC(C)(C)C)CC3(C)C)oc12 ; CC(C)c1cc(-c2ccc(F)cc2)nc2cc(C(=O)N3CCN(C(=O)C4(C)CC4)CC3(C)C)oc12 ; CC#CC(=O)N1CCN(C(=O)c2cc3nc(-c4ccc(F)cc4)cc(C(C)C)c3o2)C(C)(C)C1]

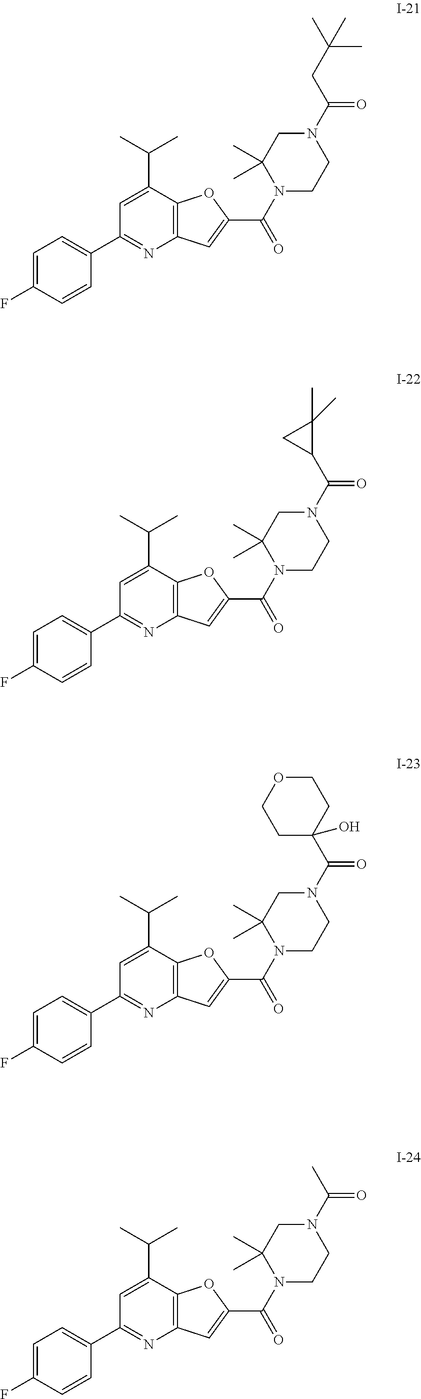 [SMILES: CC(C)c1cc(-c2ccc(F)cc2)nc2cc(C(=O)N3CCN(C(=O)CC(C)(C)C)CC3(C)C)oc12 ; CC(C)c1cc(-c2ccc(F)cc2)nc2cc(C(=O)N3CCN(C(=O)C4CC4(C)C)CC3(C)C)oc12 ; CC(C)c1cc(-c2ccc(F)cc2)nc2cc(C(=O)N3CCN(C(=O)C4(O)CCOCC4)CC3(C)C)oc12 ; CC(=O)N1CCN(C(=O)c2cc3nc(-c4ccc(F)cc4)cc(C(C)C)c3o2)C(C)(C)C1]

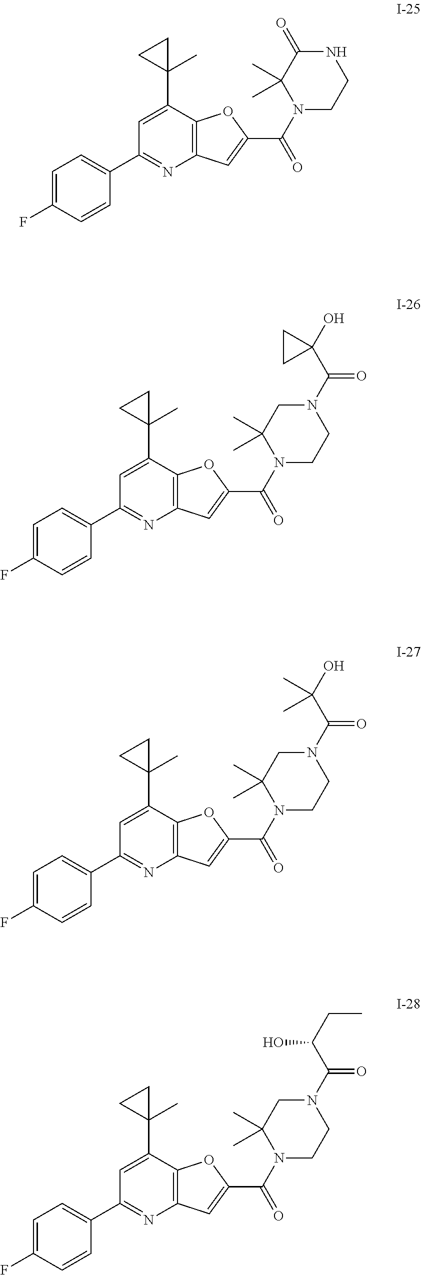 [SMILES: CC1(c2cc(-c3ccc(F)cc3)nc3cc(C(=O)N4CCNC(=O)C4(C)C)oc23)CC1 ; CC1(c2cc(-c3ccc(F)cc3)nc3cc(C(=O)N4CCN(C(=O)C5(O)CC5)CC4(C)C)oc23)CC1 ; CC(C)(O)C(=O)N1CCN(C(=O)c2cc3nc(-c4ccc(F)cc4)cc(C4(C)CC4)c3o2)C(C)(C)C1 ; CC[C@@H](O)C(=O)N1CCN(C(=O)c2cc3nc(-c4ccc(F)cc4)cc(C4(C)CC4)c3o2)C(C)(C)C1]

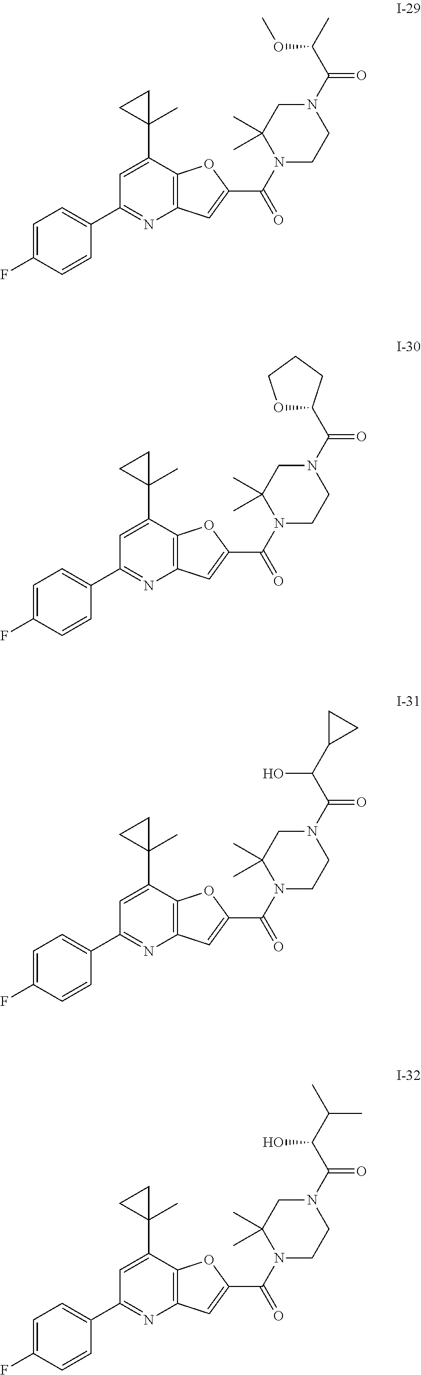 [SMILES: CO[C@H](C)C(=O)N1CCN(C(=O)c2cc3nc(-c4ccc(F)cc4)cc(C4(C)CC4)c3o2)C(C)(C)C1 ; CC1(c2cc(-c3ccc(F)cc3)nc3cc(C(=O)N4CCN(C(=O)[C@H]5CCCO5)CC4(C)C)oc23)CC1 ; CC1(c2cc(-c3ccc(F)cc3)nc3cc(C(=O)N4CCN(C(=O)C(O)C5CC5)CC4(C)C)oc23)CC1 ; CC(C)[C@@H](O)C(=O)N1CCN(C(=O)c2cc3nc(-c4ccc(F)cc4)cc(C4(C)CC4)c3o2)C(C)(C)C1]

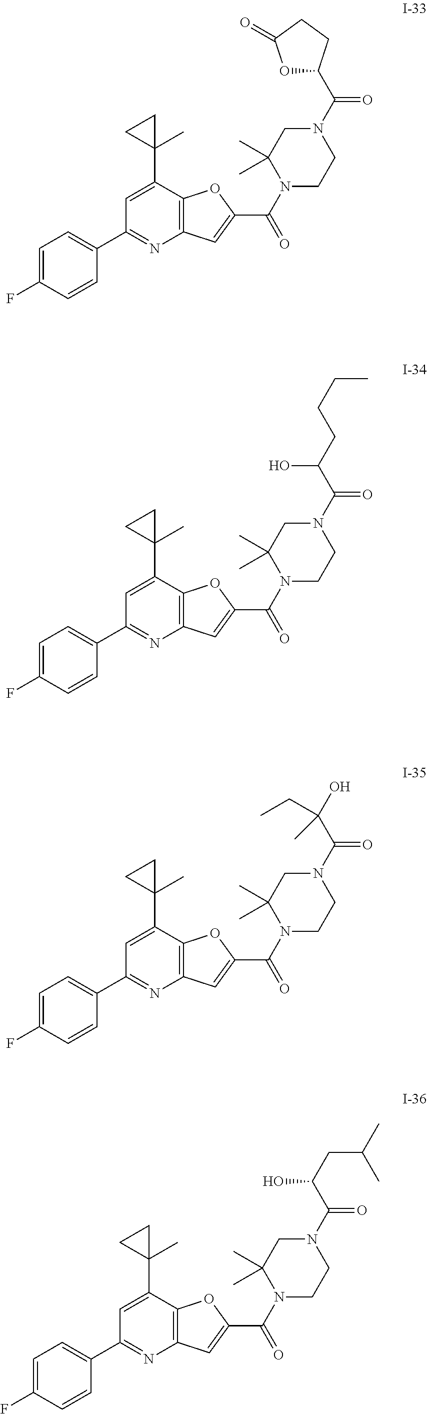 [SMILES: CC1(c2cc(-c3ccc(F)cc3)nc3cc(C(=O)N4CCN(C(=O)[C@H]5CCC(=O)O5)CC4(C)C)oc23)CC1 ; CCCCC(O)C(=O)N1CCN(C(=O)c2cc3nc(-c4ccc(F)cc4)cc(C4(C)CC4)c3o2)C(C)(C)C1 ; CCC(C)(O)C(=O)N1CCN(C(=O)c2cc3nc(-c4ccc(F)cc4)cc(C4(C)CC4)c3o2)C(C)(C)C1 ; CC(C)C[C@@H](O)C(=O)N1CCN(C(=O)c2cc3nc(-c4ccc(F)cc4)cc(C4(C)CC4)c3o2)C(C)(C)C1]

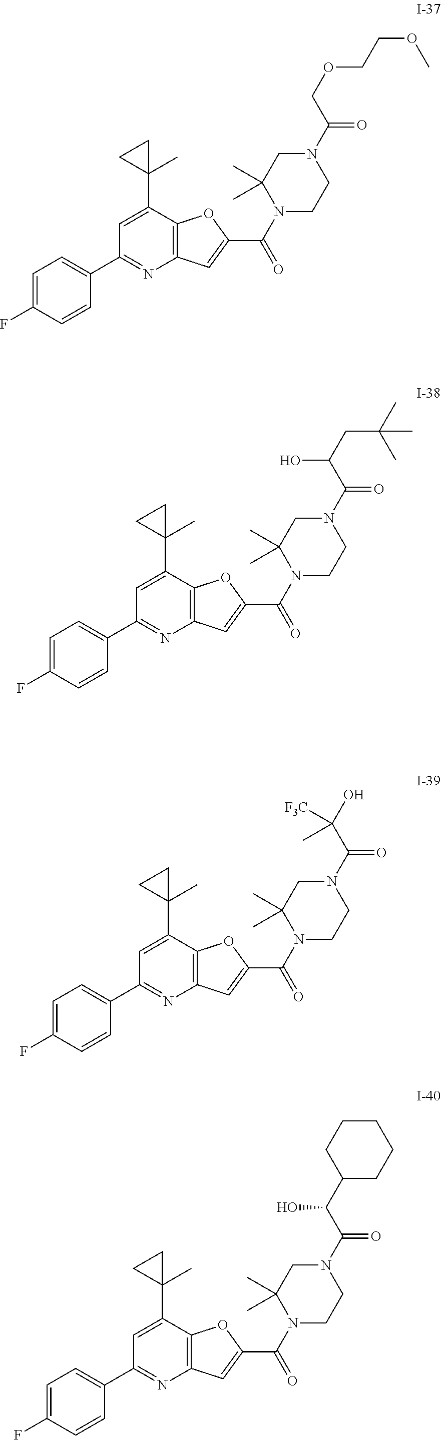 [SMILES: COCCOCC(=O)N1CCN(C(=O)c2cc3nc(-c4ccc(F)cc4)cc(C4(C)CC4)c3o2)C(C)(C)C1 ; CC(C)(C)CC(O)C(=O)N1CCN(C(=O)c2cc3nc(-c4ccc(F)cc4)cc(C4(C)CC4)c3o2)C(C)(C)C1 ; CC1(c2cc(-c3ccc(F)cc3)nc3cc(C(=O)N4CCN(C(=O)C(C)(O)C(F)(F)F)CC4(C)C)oc23)CC1 ; CC1(c2cc(-c3ccc(F)cc3)nc3cc(C(=O)N4CCN(C(=O)[C@H](O)C5CCCCC5)CC4(C)C)oc23)CC1]

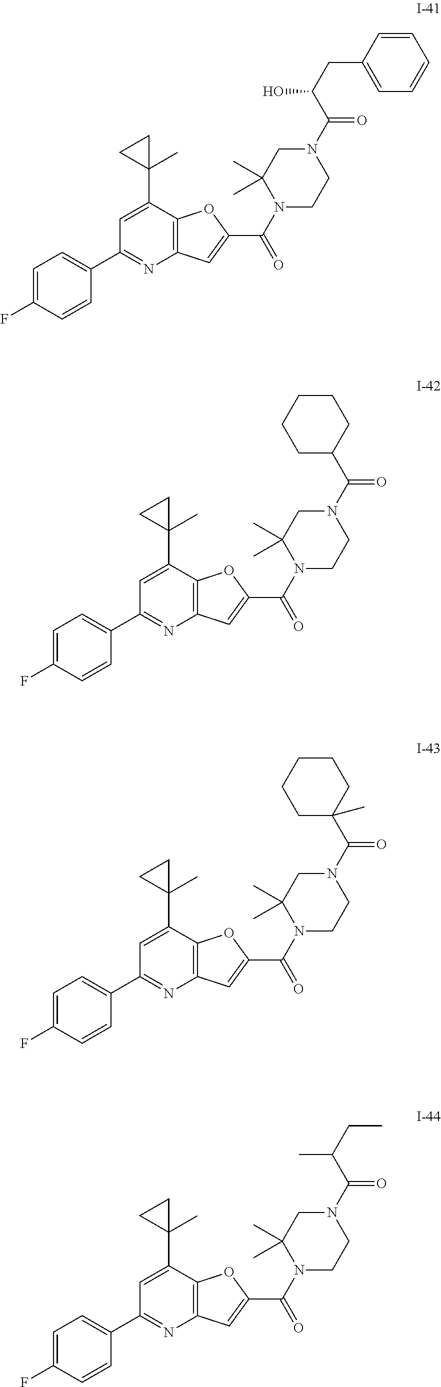 [SMILES: CC1(c2cc(-c3ccc(F)cc3)nc3cc(C(=O)N4CCN(C(=O)[C@H](O)Cc5ccccc5)CC4(C)C)oc23)CC1 ; CC1(c2cc(-c3ccc(F)cc3)nc3cc(C(=O)N4CCN(C(=O)C5CCCCC5)CC4(C)C)oc23)CC1 ; CC1(C(=O)N2CCN(C(=O)c3cc4nc(-c5ccc(F)cc5)cc(C5(C)CC5)c4o3)C(C)(C)C2)CCCCC1 ; CCC(C)C(=O)N1CCN(C(=O)c2cc3nc(-c4ccc(F)cc4)cc(C4(C)CC4)c3o2)C(C)(C)C1]

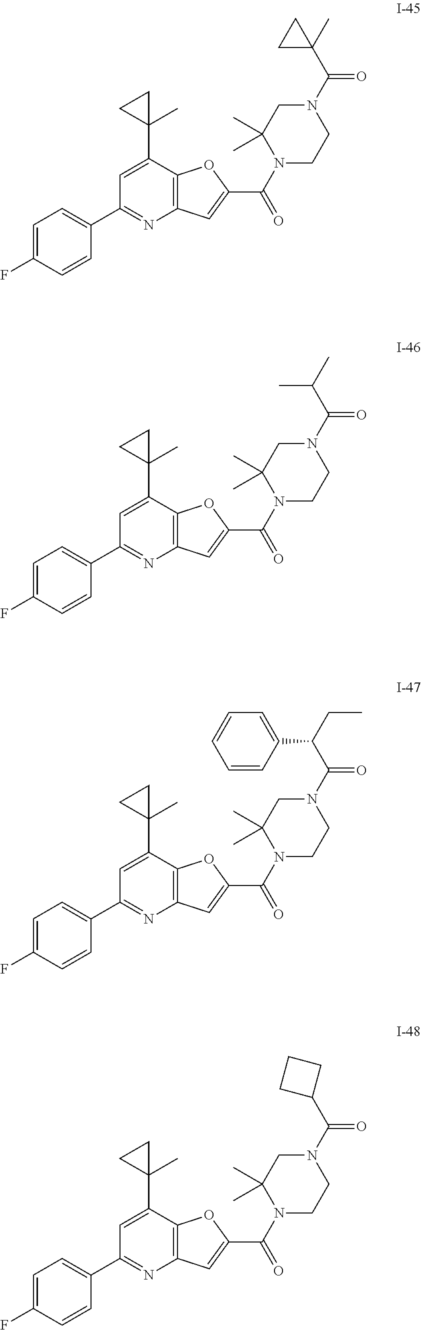 [SMILES: CC1(C(=O)N2CCN(C(=O)c3cc4nc(-c5ccc(F)cc5)cc(C5(C)CC5)c4o3)C(C)(C)C2)CC1 ; CC(C)C(=O)N1CCN(C(=O)c2cc3nc(-c4ccc(F)cc4)cc(C4(C)CC4)c3o2)C(C)(C)C1 ; CC[C@H](C(=O)N1CCN(C(=O)c2cc3nc(-c4ccc(F)cc4)cc(C4(C)CC4)c3o2)C(C)(C)C1)c1ccccc1 ; CC1(c2cc(-c3ccc(F)cc3)nc3cc(C(=O)N4CCN(C(=O)C5CCC5)CC4(C)C)oc23)CC1]

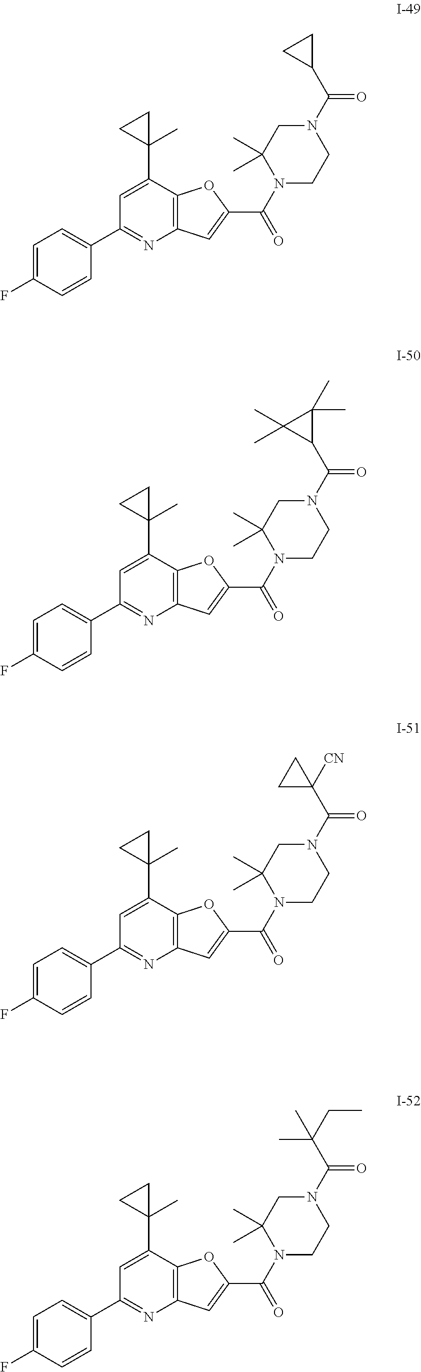 [SMILES: CC1(c2cc(-c3ccc(F)cc3)nc3cc(C(=O)N4CCN(C(=O)C5CC5)CC4(C)C)oc23)CC1 ; CC1(c2cc(-c3ccc(F)cc3)nc3cc(C(=O)N4CCN(C(=O)C5C(C)(C)C5(C)C)CC4(C)C)oc23)CC1 ; CC1(c2cc(-c3ccc(F)cc3)nc3cc(C(=O)N4CCN(C(=O)C5(C#N)CC5)CC4(C)C)oc23)CC1 ; CCC(C)(C)C(=O)N1CCN(C(=O)c2cc3nc(-c4ccc(F)cc4)cc(C4(C)CC4)c3o2)C(C)(C)C1]

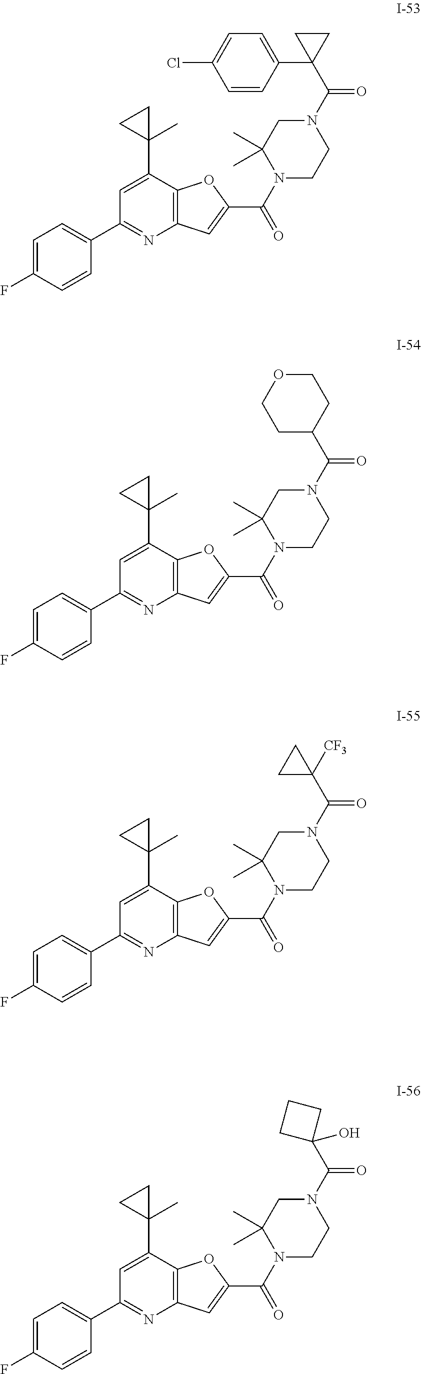 [SMILES: CC1(c2cc(-c3ccc(F)cc3)nc3cc(C(=O)N4CCN(C(=O)C5(c6ccc(Cl)cc6)CC5)CC4(C)C)oc23)CC1 ; CC1(c2cc(-c3ccc(F)cc3)nc3cc(C(=O)N4CCN(C(=O)C5CCOCC5)CC4(C)C)oc23)CC1 ; CC1(c2cc(-c3ccc(F)cc3)nc3cc(C(=O)N4CCN(C(=O)C5(C(F)(F)F)CC5)CC4(C)C)oc23)CC1 ; CC1(c2cc(-c3ccc(F)cc3)nc3cc(C(=O)N4CCN(C(=O)C5(O)CCC5)CC4(C)C)oc23)CC1]

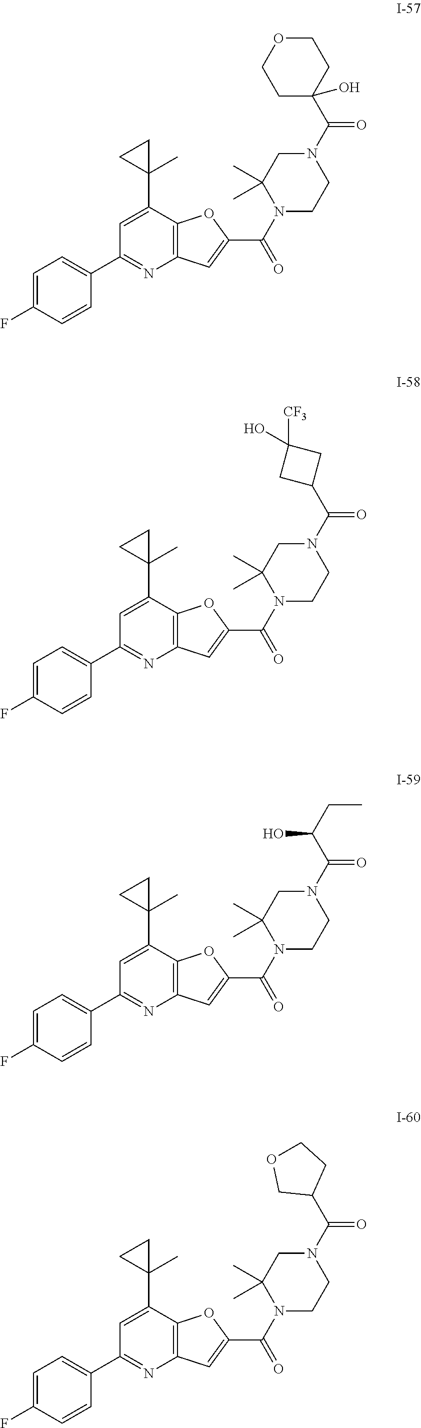 [SMILES: CC1(c2cc(-c3ccc(F)cc3)nc3cc(C(=O)N4CCN(C(=O)C5(O)CCOCC5)CC4(C)C)oc23)CC1 ; CC1(c2cc(-c3ccc(F)cc3)nc3cc(C(=O)N4CCN(C(=O)C5CC(O)(C(F)(F)F)C5)CC4(C)C)oc23)CC1 ; CC[C@H](O)C(=O)N1CCN(C(=O)c2cc3nc(-c4ccc(F)cc4)cc(C4(C)CC4)c3o2)C(C)(C)C1 ; CC1(c2cc(-c3ccc(F)cc3)nc3cc(C(=O)N4CCN(C(=O)C5CCOC5)CC4(C)C)oc23)CC1]

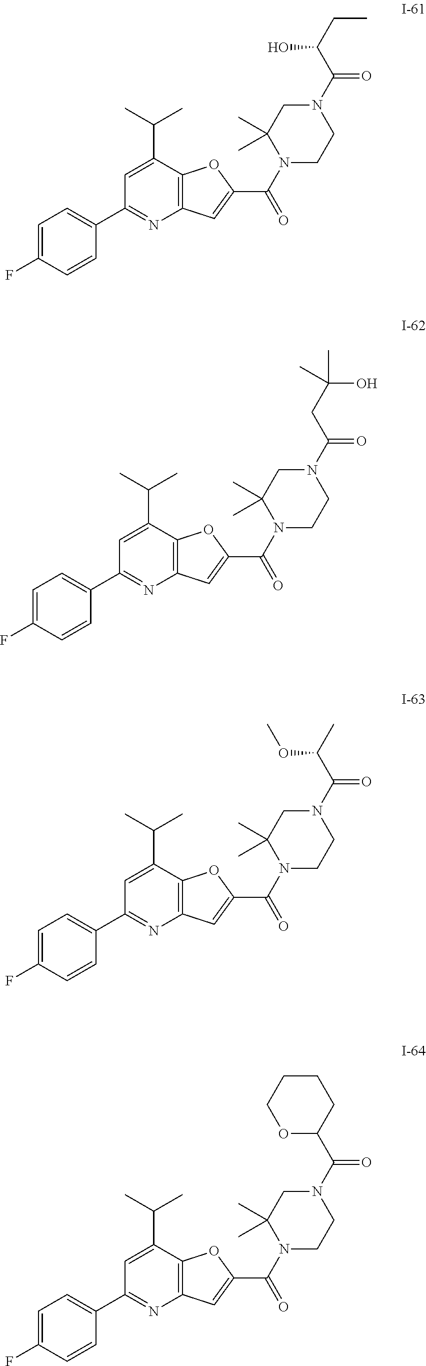 [SMILES: CC[C@@H](O)C(=O)N1CCN(C(=O)c2cc3nc(-c4ccc(F)cc4)cc(C(C)C)c3o2)C(C)(C)C1 ; CC(C)c1cc(-c2ccc(F)cc2)nc2cc(C(=O)N3CCN(C(=O)CC(C)(C)O)CC3(C)C)oc12 ; CO[C@H](C)C(=O)N1CCN(C(=O)c2cc3nc(-c4ccc(F)cc4)cc(C(C)C)c3o2)C(C)(C)C1 ; CC(C)c1cc(-c2ccc(F)cc2)nc2cc(C(=O)N3CCN(C(=O)C4CCCCO4)CC3(C)C)oc12]

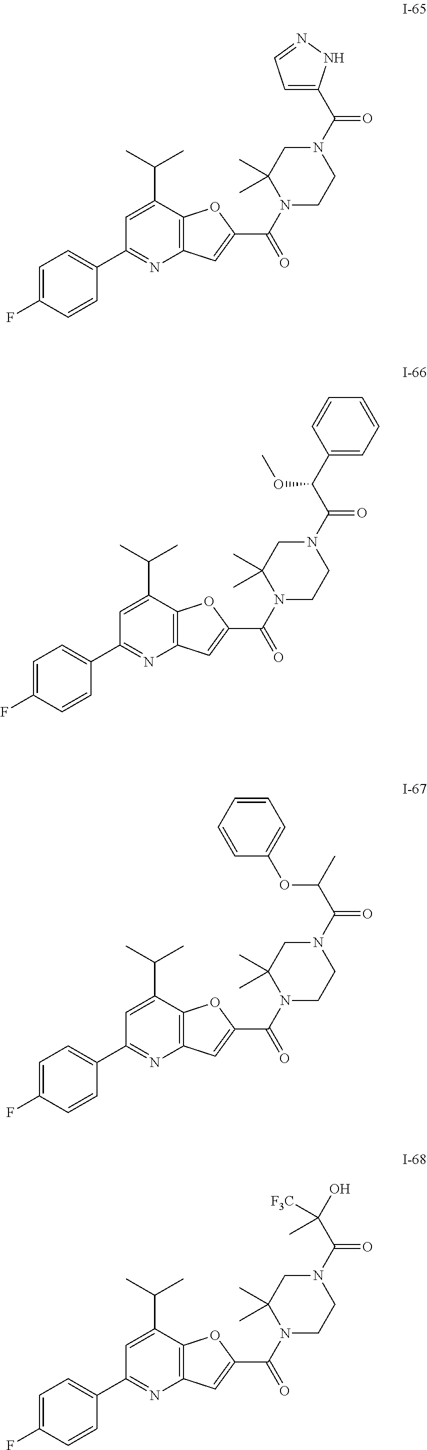 [SMILES: CC(C)c1cc(-c2ccc(F)cc2)nc2cc(C(=O)N3CCN(C(=O)c4ccn[nH]4)CC3(C)C)oc12 ; CO[C@@H](C(=O)N1CCN(C(=O)c2cc3nc(-c4ccc(F)cc4)cc(C(C)C)c3o2)C(C)(C)C1)c1ccccc1 ; CC(Oc1ccccc1)C(=O)N1CCN(C(=O)c2cc3nc(-c4ccc(F)cc4)cc(C(C)C)c3o2)C(C)(C)C1 ; CC(C)c1cc(-c2ccc(F)cc2)nc2cc(C(=O)N3CCN(C(=O)C(C)(O)C(F)(F)F)CC3(C)C)oc12]

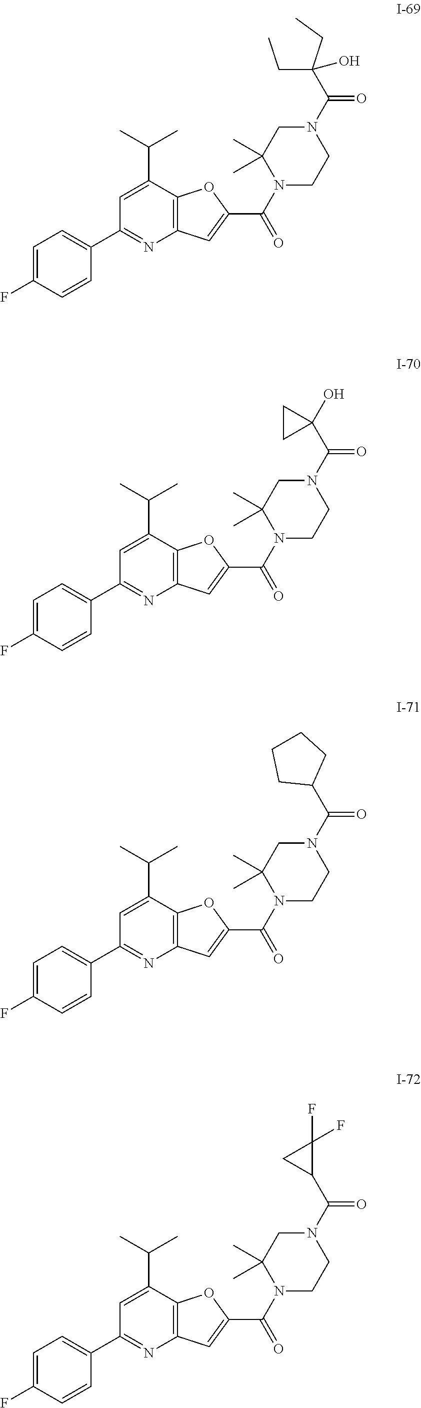 [SMILES: CCC(O)(CC)C(=O)N1CCN(C(=O)c2cc3nc(-c4ccc(F)cc4)cc(C(C)C)c3o2)C(C)(C)C1 ; CC(C)c1cc(-c2ccc(F)cc2)nc2cc(C(=O)N3CCN(C(=O)C4(O)CC4)CC3(C)C)oc12 ; CC(C)c1cc(-c2ccc(F)cc2)nc2cc(C(=O)N3CCN(C(=O)C4CCCC4)CC3(C)C)oc12 ; CC(C)c1cc(-c2ccc(F)cc2)nc2cc(C(=O)N3CCN(C(=O)C4CC4(F)F)CC3(C)C)oc12]

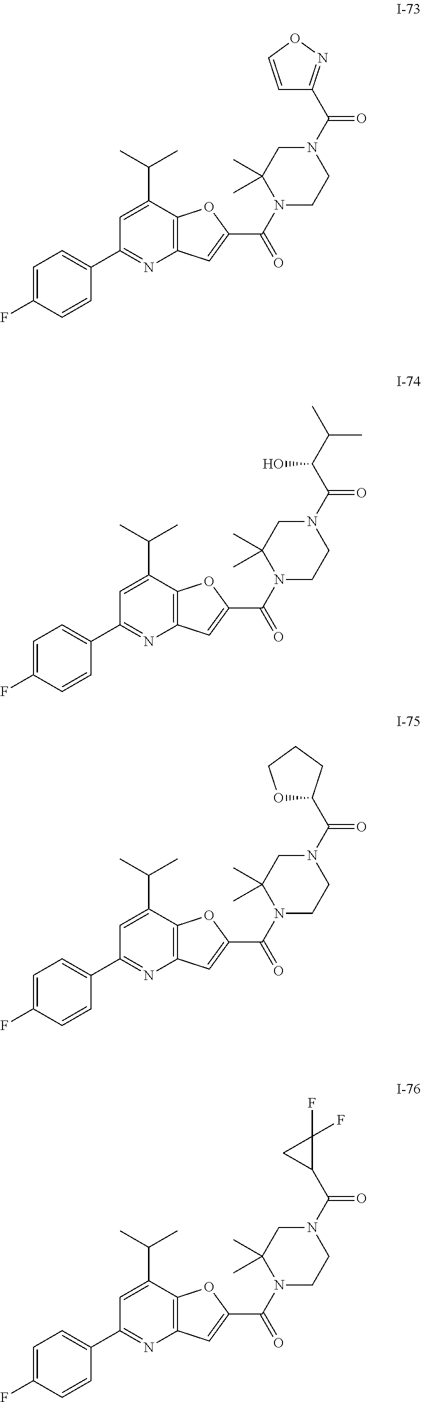 [SMILES: CC(C)c1cc(-c2ccc(F)cc2)nc2cc(C(=O)N3CCN(C(=O)c4ccon4)CC3(C)C)oc12 ; CC(C)c1cc(-c2ccc(F)cc2)nc2cc(C(=O)N3CCN(C(=O)[C@H](O)C(C)C)CC3(C)C)oc12 ; CC(C)c1cc(-c2ccc(F)cc2)nc2cc(C(=O)N3CCN(C(=O)[C@H]4CCCO4)CC3(C)C)oc12 ; CC(C)c1cc(-c2ccc(F)cc2)nc2cc(C(=O)N3CCN(C(=O)C4CC4(F)F)CC3(C)C)oc12]

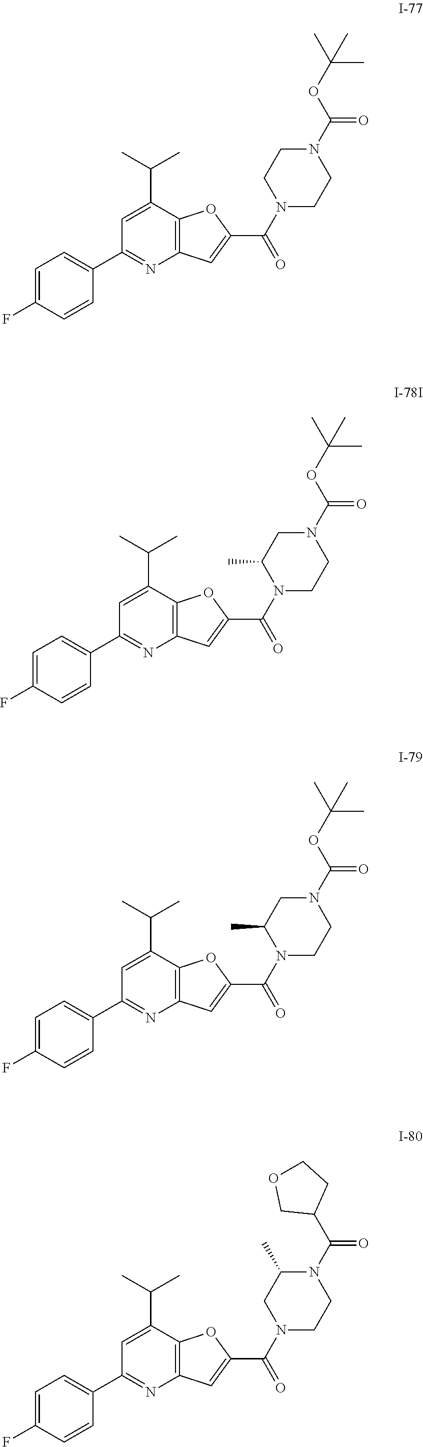 [SMILES: CC(C)c1cc(-c2ccc(F)cc2)nc2cc(C(=O)N3CCN(C(=O)OC(C)(C)C)CC3)oc12 ; CC(C)c1cc(-c2ccc(F)cc2)nc2cc(C(=O)N3CCN(C(=O)OC(C)(C)C)C[C@H]3C)oc12 ; CC(C)c1cc(-c2ccc(F)cc2)nc2cc(C(=O)N3CCN(C(=O)OC(C)(C)C)C[C@@H]3C)oc12 ; CC(C)c1cc(-c2ccc(F)cc2)nc2cc(C(=O)N3CCN(C(=O)C4CCOC4)[C@@H](C)C3)oc12]

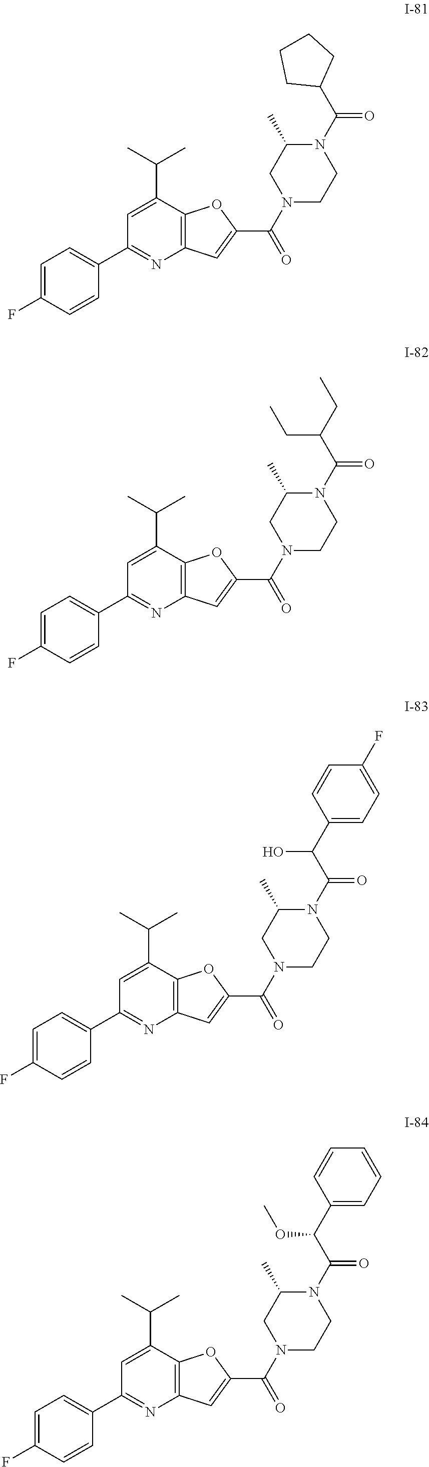 [SMILES: CC(C)c1cc(-c2ccc(F)cc2)nc2cc(C(=O)N3CCN(C(=O)C4CCCC4)[C@@H](C)C3)oc12 ; CCC(CC)C(=O)N1CCN(C(=O)c2cc3nc(-c4ccc(F)cc4)cc(C(C)C)c3o2)C[C@@H]1C ; CC(C)c1cc(-c2ccc(F)cc2)nc2cc(C(=O)N3CCN(C(=O)C(O)c4ccc(F)cc4)[C@@H](C)C3)oc12 ; CO[C@@H](C(=O)N1CCN(C(=O)c2cc3nc(-c4ccc(F)cc4)cc(C(C)C)c3o2)C[C@@H]1C)c1ccccc1]

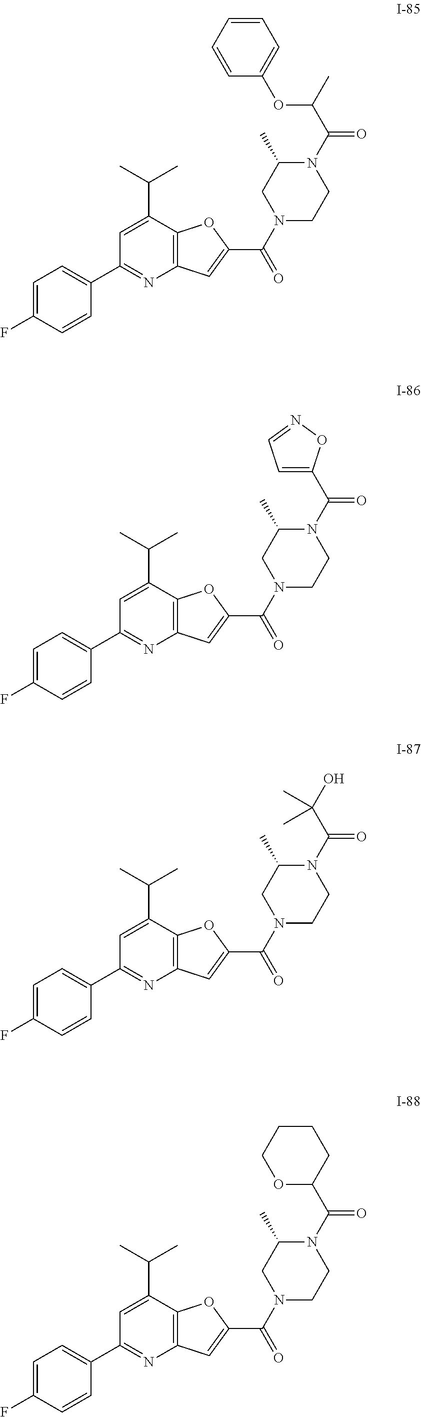 [SMILES: CC(Oc1ccccc1)C(=O)N1CCN(C(=O)c2cc3nc(-c4ccc(F)cc4)cc(C(C)C)c3o2)C[C@@H]1C ; CC(C)c1cc(-c2ccc(F)cc2)nc2cc(C(=O)N3CCN(C(=O)c4ccno4)[C@@H](C)C3)oc12 ; CC(C)c1cc(-c2ccc(F)cc2)nc2cc(C(=O)N3CCN(C(=O)C(C)(C)O)[C@@H](C)C3)oc12 ; CC(C)c1cc(-c2ccc(F)cc2)nc2cc(C(=O)N3CCN(C(=O)C4CCCCO4)[C@@H](C)C3)oc12]

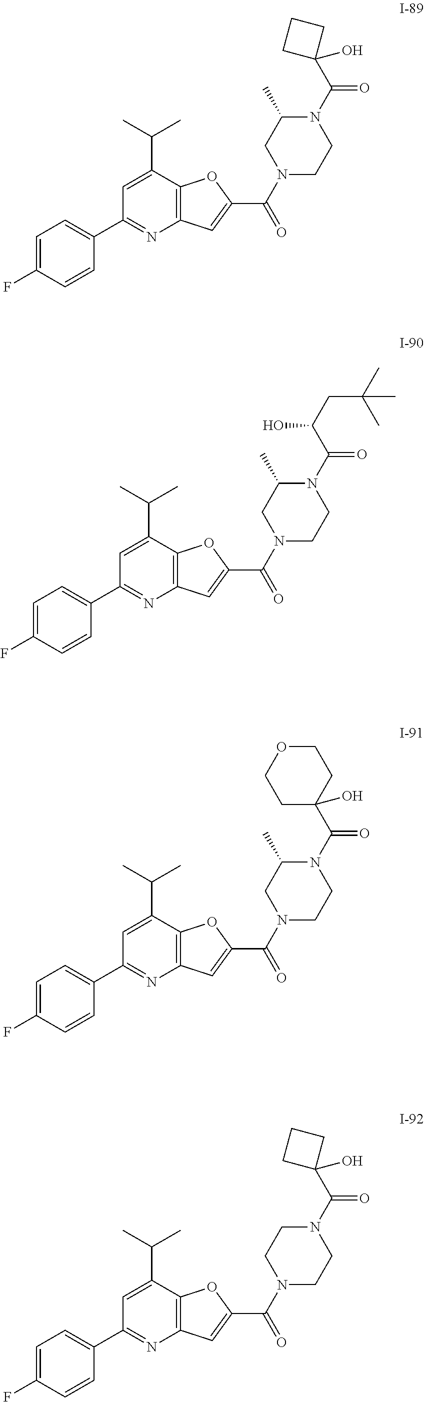 [SMILES: CC(C)c1cc(-c2ccc(F)cc2)nc2cc(C(=O)N3CCN(C(=O)C4(O)CCC4)[C@@H](C)C3)oc12 ; CC(C)c1cc(-c2ccc(F)cc2)nc2cc(C(=O)N3CCN(C(=O)[C@H](O)CC(C)(C)C)[C@@H](C)C3)oc12 ; CC(C)c1cc(-c2ccc(F)cc2)nc2cc(C(=O)N3CCN(C(=O)C4(O)CCOCC4)[C@@H](C)C3)oc12 ; CC(C)c1cc(-c2ccc(F)cc2)nc2cc(C(=O)N3CCN(C(=O)C4(O)CCC4)CC3)oc12]

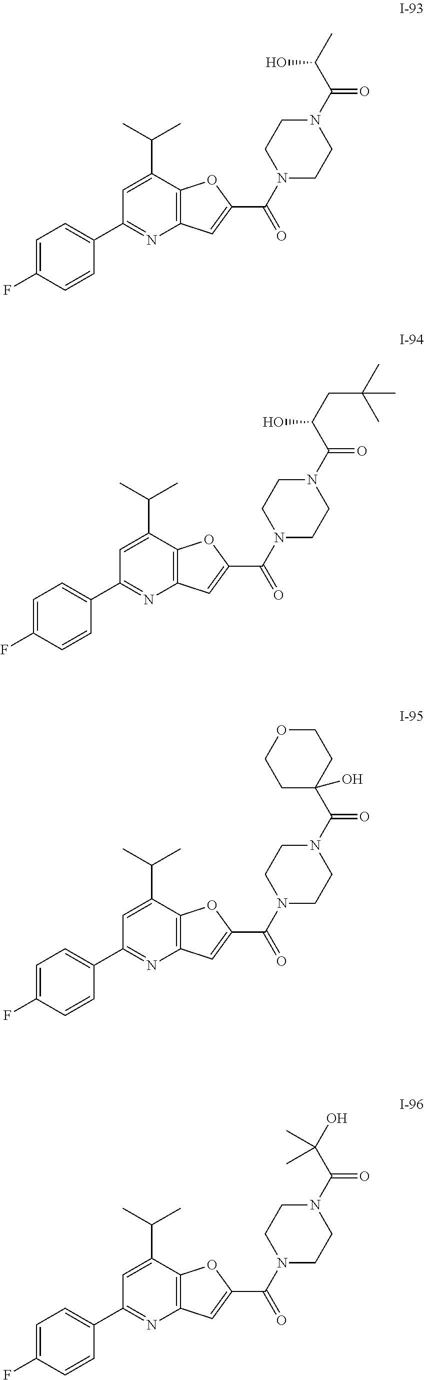 [SMILES: CC(C)c1cc(-c2ccc(F)cc2)nc2cc(C(=O)N3CCN(C(=O)[C@@H](C)O)CC3)oc12 ; CC(C)c1cc(-c2ccc(F)cc2)nc2cc(C(=O)N3CCN(C(=O)[C@H](O)CC(C)(C)C)CC3)oc12 ; CC(C)c1cc(-c2ccc(F)cc2)nc2cc(C(=O)N3CCN(C(=O)C4(O)CCOCC4)CC3)oc12 ; CC(C)c1cc(-c2ccc(F)cc2)nc2cc(C(=O)N3CCN(C(=O)C(C)(C)O)CC3)oc12]

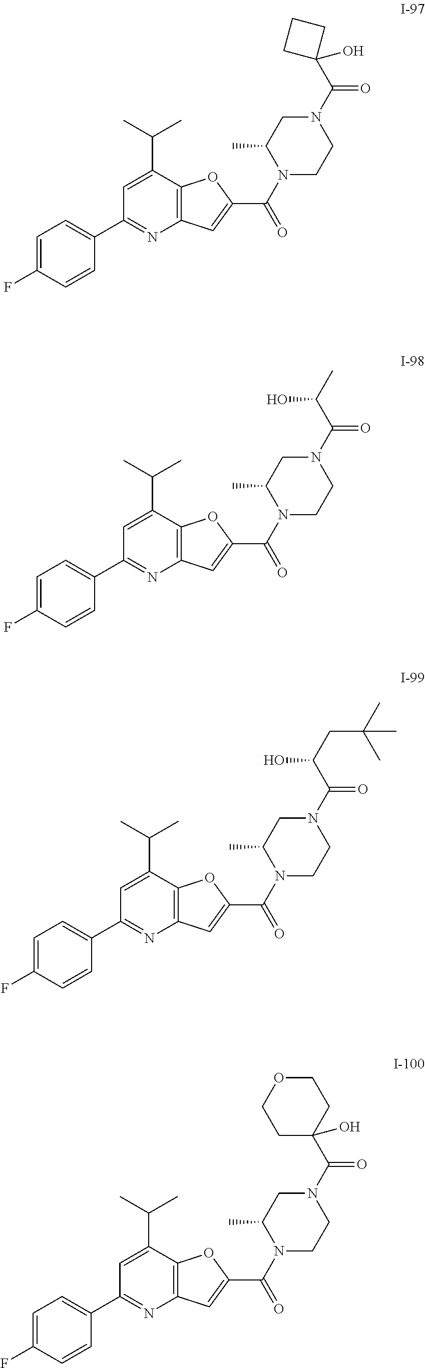 [SMILES: CC(C)c1cc(-c2ccc(F)cc2)nc2cc(C(=O)N3CCN(C(=O)C4(O)CCC4)C[C@H]3C)oc12 ; CC(C)c1cc(-c2ccc(F)cc2)nc2cc(C(=O)N3CCN(C(=O)[C@@H](C)O)C[C@H]3C)oc12 ; CC(C)c1cc(-c2ccc(F)cc2)nc2cc(C(=O)N3CCN(C(=O)[C@H](O)CC(C)(C)C)C[C@H]3C)oc12 ; CC(C)c1cc(-c2ccc(F)cc2)nc2cc(C(=O)N3CCN(C(=O)C4(O)CCOCC4)C[C@H]3C)oc12]

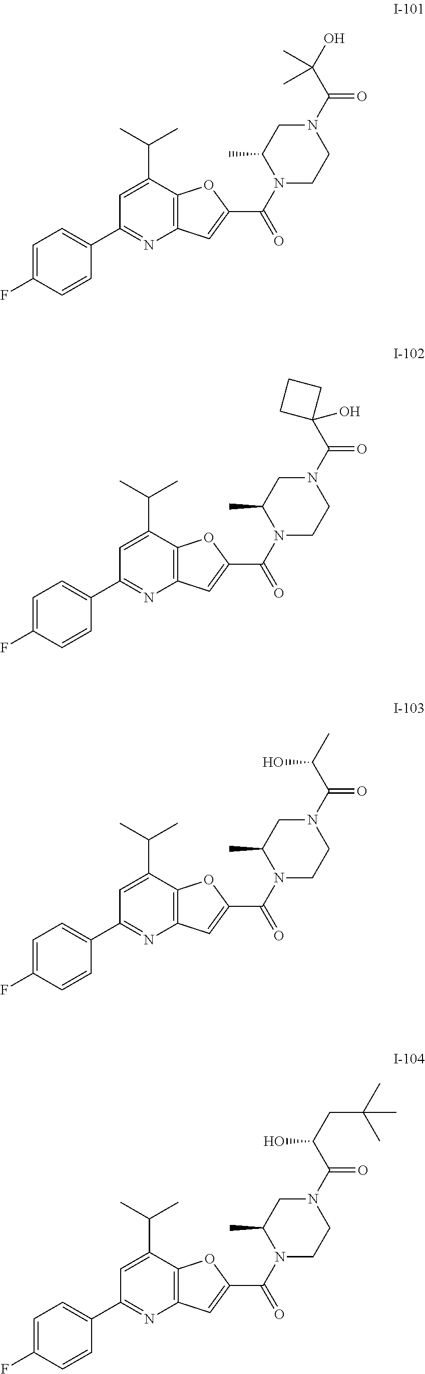 [SMILES: CC(C)c1cc(-c2ccc(F)cc2)nc2cc(C(=O)N3CCN(C(=O)C(C)(C)O)C[C@H]3C)oc12 ; CC(C)c1cc(-c2ccc(F)cc2)nc2cc(C(=O)N3CCN(C(=O)C4(O)CCC4)C[C@@H]3C)oc12 ; CC(C)c1cc(-c2ccc(F)cc2)nc2cc(C(=O)N3CCN(C(=O)[C@@H](C)O)C[C@@H]3C)oc12 ; CC(C)c1cc(-c2ccc(F)cc2)nc2cc(C(=O)N3CCN(C(=O)[C@H](O)CC(C)(C)C)C[C@@H]3C)oc12]

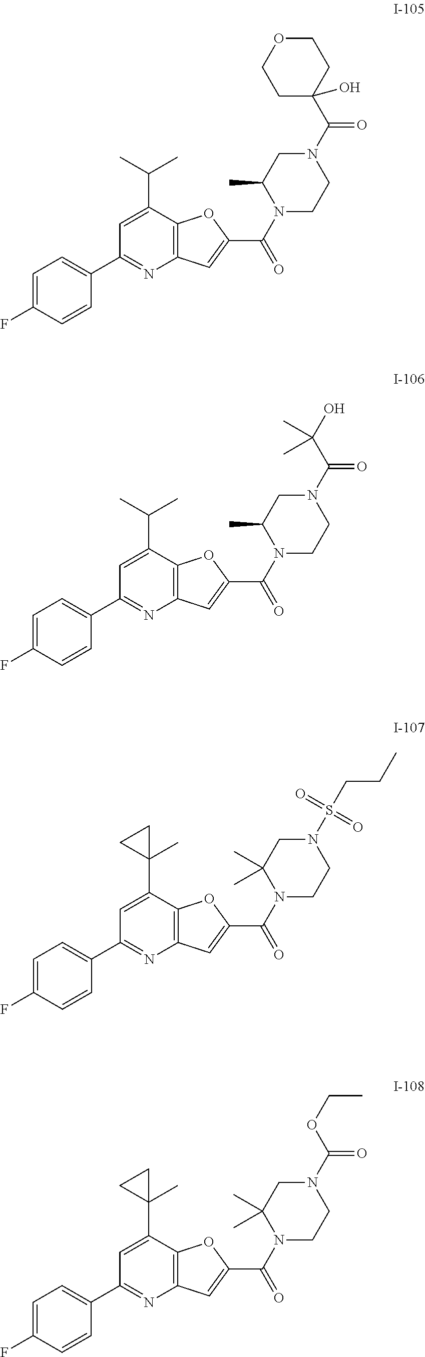 [SMILES: CC(C)c1cc(-c2ccc(F)cc2)nc2cc(C(=O)N3CCN(C(=O)C4(O)CCOCC4)C[C@@H]3C)oc12 ; CC(C)c1cc(-c2ccc(F)cc2)nc2cc(C(=O)N3CCN(C(=O)C(C)(C)O)C[C@@H]3C)oc12 ; CCCS(=O)(=O)N1CCN(C(=O)c2cc3nc(-c4ccc(F)cc4)cc(C4(C)CC4)c3o2)C(C)(C)C1 ; CCOC(=O)N1CCN(C(=O)c2cc3nc(-c4ccc(F)cc4)cc(C4(C)CC4)c3o2)C(C)(C)C1]

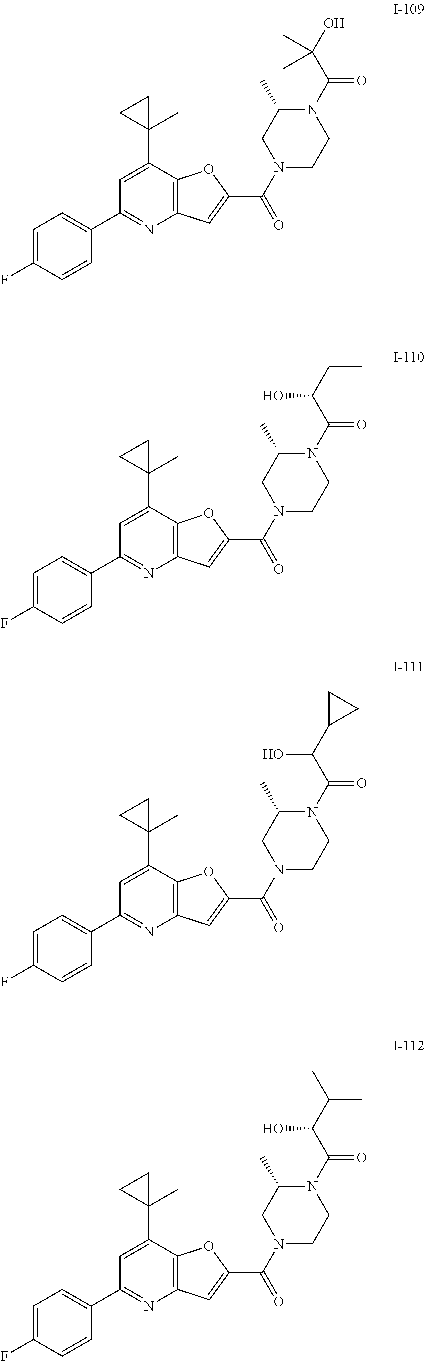 [SMILES: C[C@H]1CN(C(=O)c2cc3nc(-c4ccc(F)cc4)cc(C4(C)CC4)c3o2)CCN1C(=O)C(C)(C)O ; CC[C@@H](O)C(=O)N1CCN(C(=O)c2cc3nc(-c4ccc(F)cc4)cc(C4(C)CC4)c3o2)C[C@@H]1C ; C[C@H]1CN(C(=O)c2cc3nc(-c4ccc(F)cc4)cc(C4(C)CC4)c3o2)CCN1C(=O)C(O)C1CC1 ; CC(C)[C@@H](O)C(=O)N1CCN(C(=O)c2cc3nc(-c4ccc(F)cc4)cc(C4(C)CC4)c3o2)C[C@@H]1C]

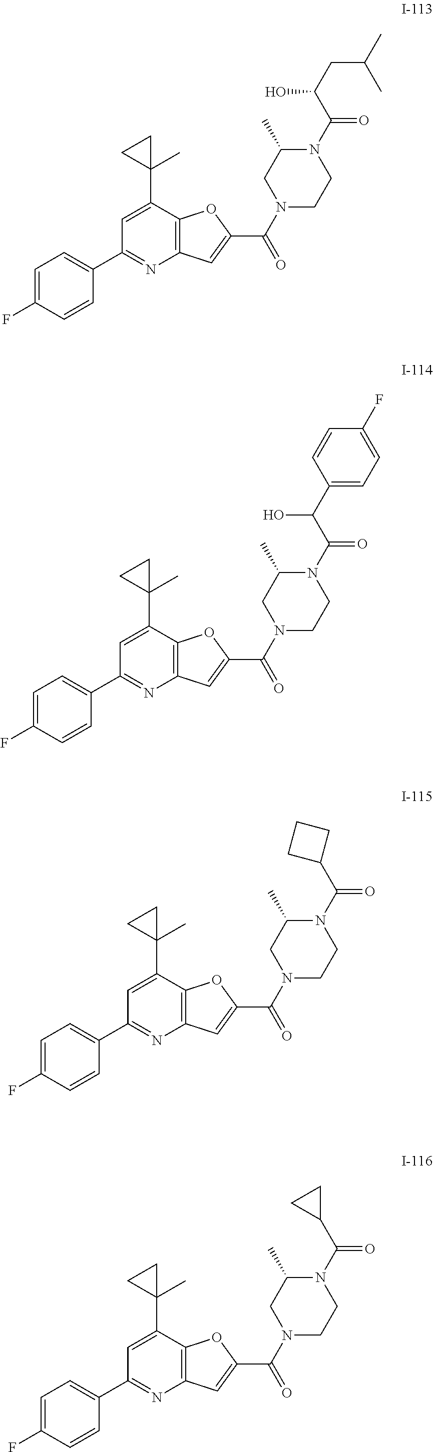 [SMILES: CC(C)C[C@@H](O)C(=O)N1CCN(C(=O)c2cc3nc(-c4ccc(F)cc4)cc(C4(C)CC4)c3o2)C[C@@H]1C ; C[C@H]1CN(C(=O)c2cc3nc(-c4ccc(F)cc4)cc(C4(C)CC4)c3o2)CCN1C(=O)C(O)c1ccc(F)cc1 ; C[C@H]1CN(C(=O)c2cc3nc(-c4ccc(F)cc4)cc(C4(C)CC4)c3o2)CCN1C(=O)C1CCC1 ; C[C@H]1CN(C(=O)c2cc3nc(-c4ccc(F)cc4)cc(C4(C)CC4)c3o2)CCN1C(=O)C1CC1]

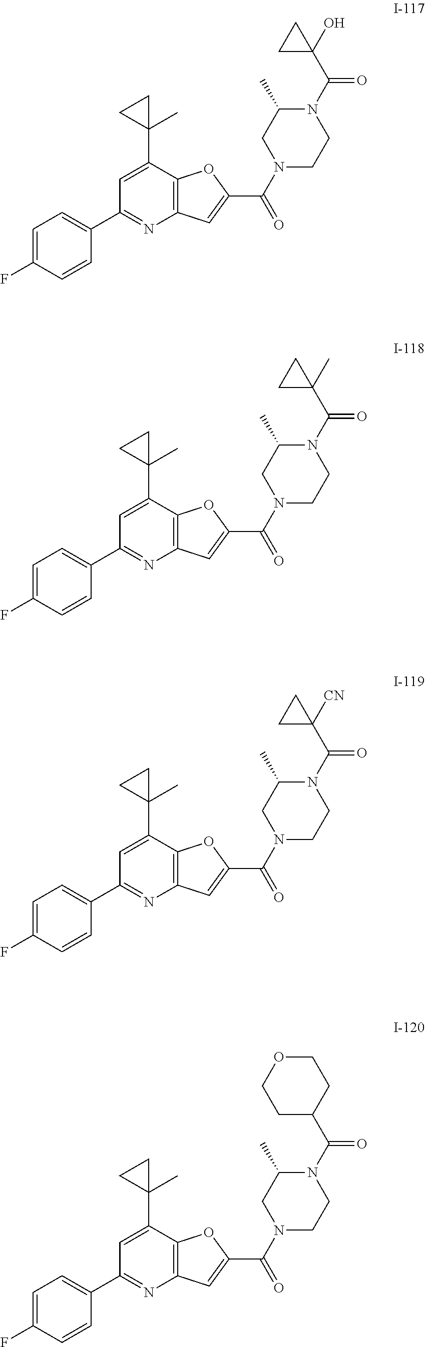 [SMILES: C[C@H]1CN(C(=O)c2cc3nc(-c4ccc(F)cc4)cc(C4(C)CC4)c3o2)CCN1C(=O)C1(O)CC1 ; C[C@H]1CN(C(=O)c2cc3nc(-c4ccc(F)cc4)cc(C4(C)CC4)c3o2)CCN1C(=O)C1(C)CC1 ; C[C@H]1CN(C(=O)c2cc3nc(-c4ccc(F)cc4)cc(C4(C)CC4)c3o2)CCN1C(=O)C1(C#N)CC1 ; C[C@H]1CN(C(=O)c2cc3nc(-c4ccc(F)cc4)cc(C4(C)CC4)c3o2)CCN1C(=O)C1CCOCC1]

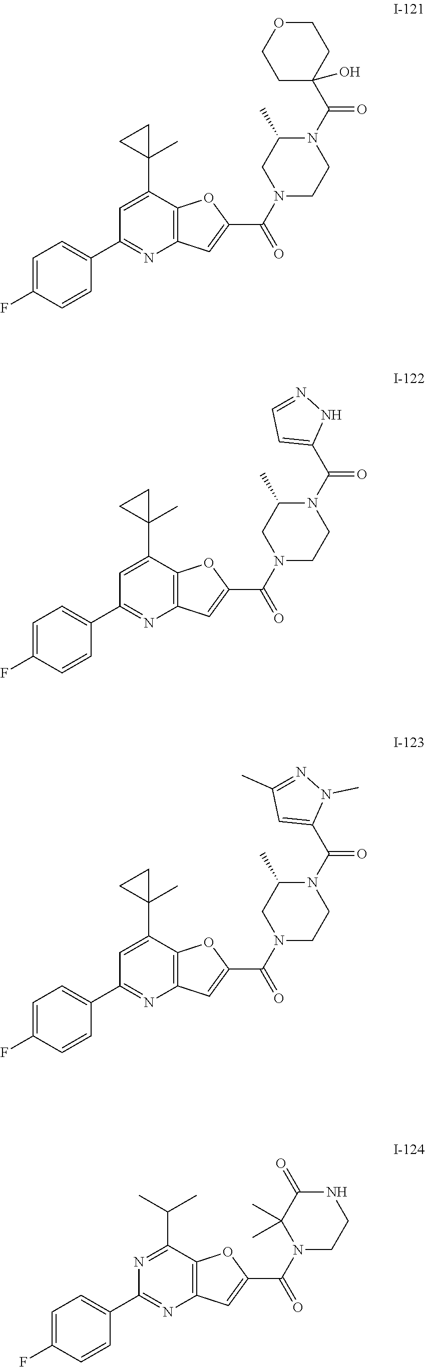 [SMILES: C[C@H]1CN(C(=O)c2cc3nc(-c4ccc(F)cc4)cc(C4(C)CC4)c3o2)CCN1C(=O)C1(O)CCOCC1 ; C[C@H]1CN(C(=O)c2cc3nc(-c4ccc(F)cc4)cc(C4(C)CC4)c3o2)CCN1C(=O)c1ccn[nH]1 ; Cc1cc(C(=O)N2CCN(C(=O)c3cc4nc(-c5ccc(F)cc5)cc(C5(C)CC5)c4o3)C[C@@H]2C)n(C)n1 ; CC(C)c1nc(-c2ccc(F)cc2)nc2cc(C(=O)N3CCNC(=O)C3(C)C)oc12]

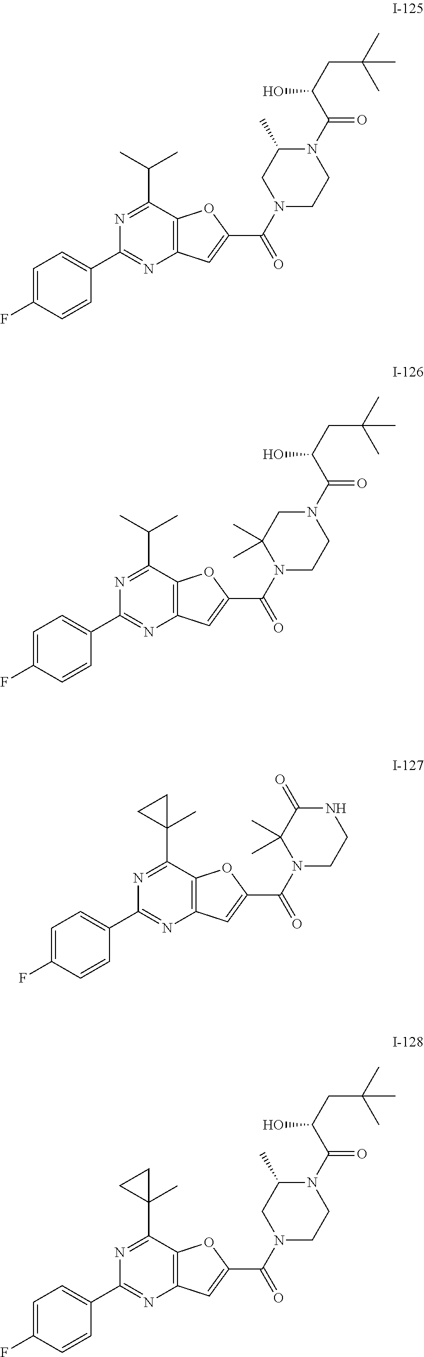 [SMILES: CC(C)c1nc(-c2ccc(F)cc2)nc2cc(C(=O)N3CCN(C(=O)[C@H](O)CC(C)(C)C)[C@@H](C)C3)oc12 ; CC(C)c1nc(-c2ccc(F)cc2)nc2cc(C(=O)N3CCN(C(=O)[C@H](O)CC(C)(C)C)CC3(C)C)oc12 ; CC1(c2nc(-c3ccc(F)cc3)nc3cc(C(=O)N4CCNC(=O)C4(C)C)oc23)CC1 ; C[C@H]1CN(C(=O)c2cc3nc(-c4ccc(F)cc4)nc(C4(C)CC4)c3o2)CCN1C(=O)[C@H](O)CC(C)(C)C]

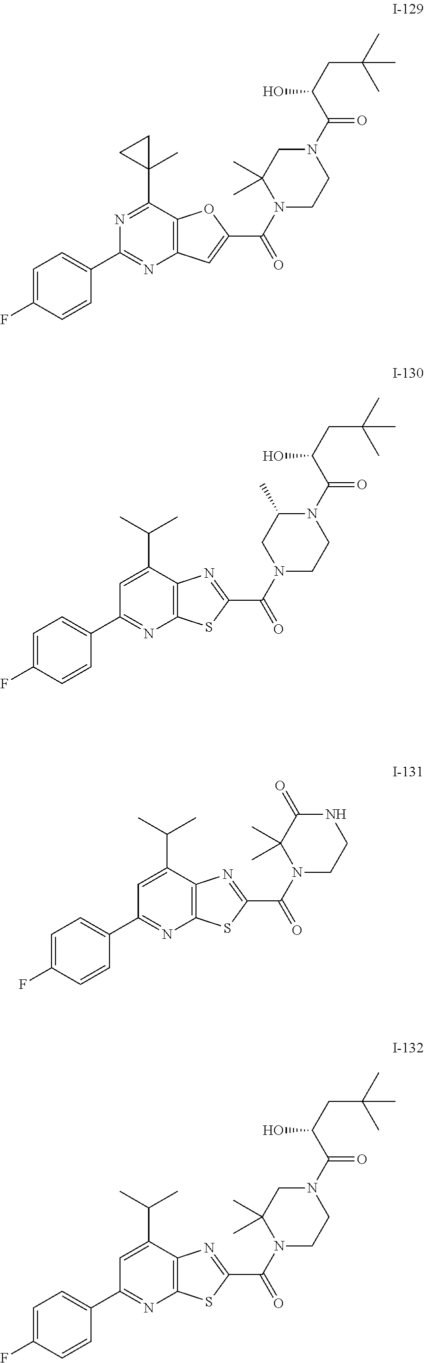 [SMILES: CC(C)(C)C[C@@H](O)C(=O)N1CCN(C(=O)c2cc3nc(-c4ccc(F)cc4)nc(C4(C)CC4)c3o2)C(C)(C)C1 ; CC(C)c1cc(-c2ccc(F)cc2)nc2sc(C(=O)N3CCN(C(=O)[C@H](O)CC(C)(C)C)[C@@H](C)C3)nc12 ; CC(C)c1cc(-c2ccc(F)cc2)nc2sc(C(=O)N3CCNC(=O)C3(C)C)nc12 ; CC(C)c1cc(-c2ccc(F)cc2)nc2sc(C(=O)N3CCN(C(=O)[C@H](O)CC(C)(C)C)CC3(C)C)nc12]

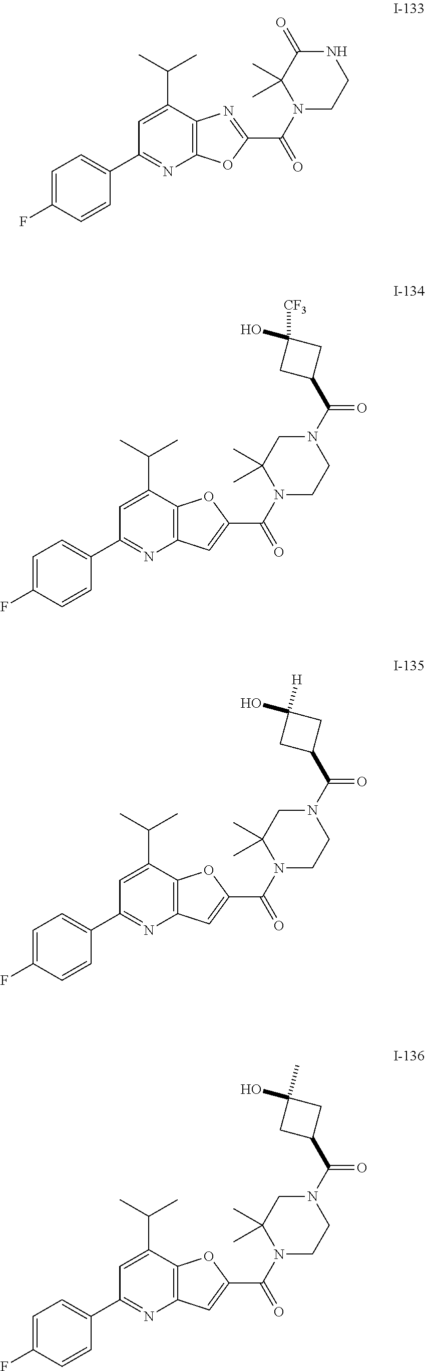

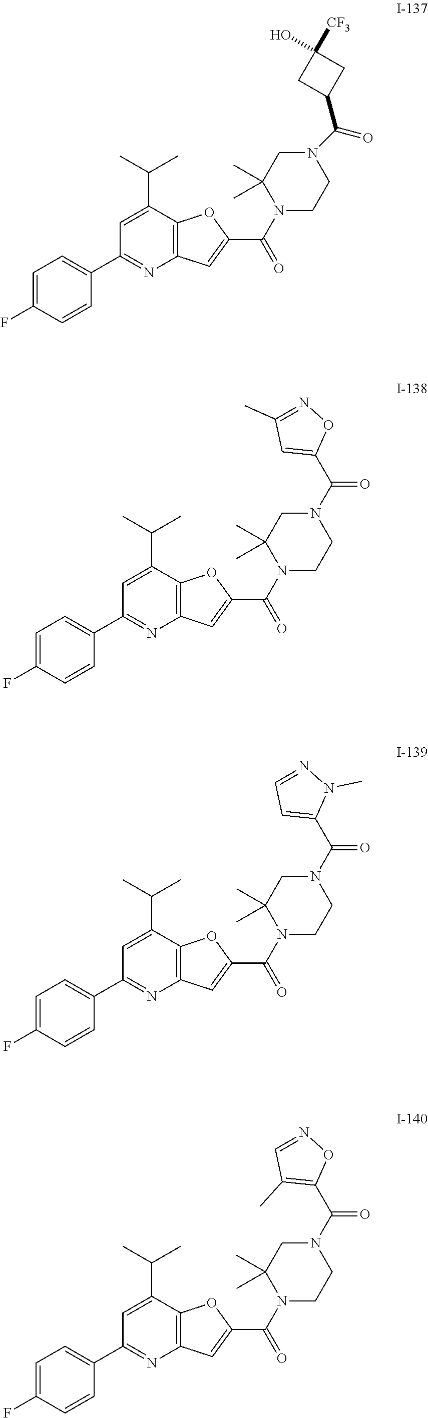

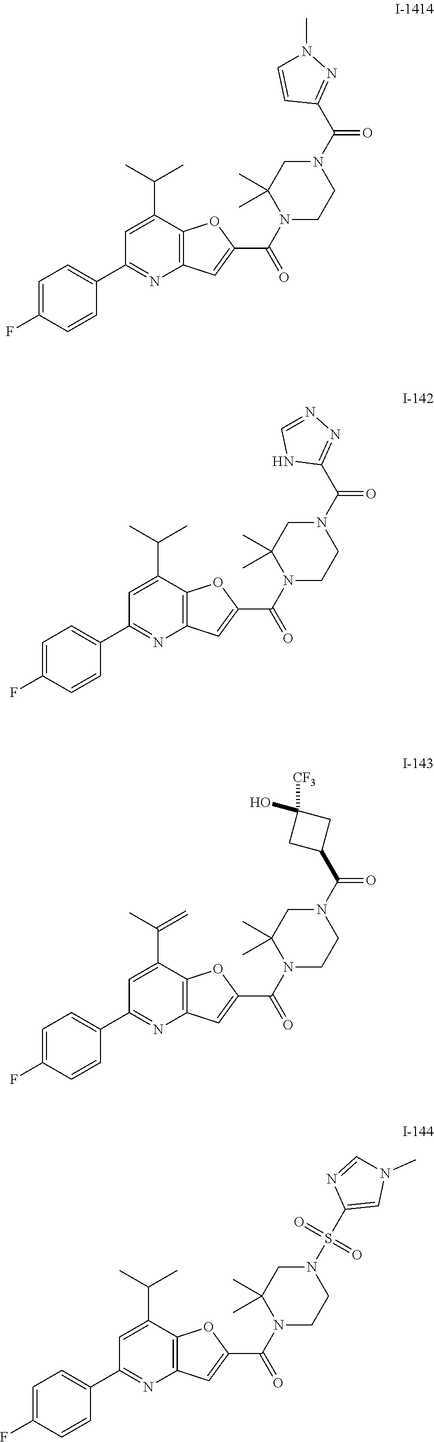

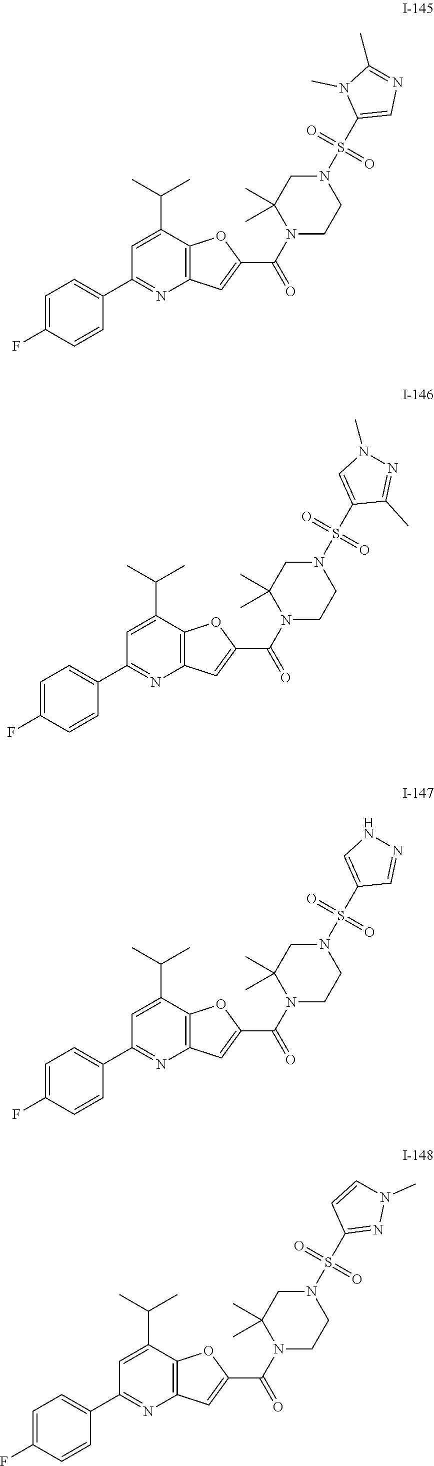 [SMILES: Cc1ncc(S(=O)(=O)N2CCN(C(=O)c3cc4nc(-c5ccc(F)cc5)cc(C(C)C)c4o3)C(C)(C)C2)n1C ; Cc1nn(C)cc1S(=O)(=O)N1CCN(C(=O)c2cc3nc(-c4ccc(F)cc4)cc(C(C)C)c3o2)C(C)(C)C1 ; CC(C)c1cc(-c2ccc(F)cc2)nc2cc(C(=O)N3CCN(S(=O)(=O)c4cn[nH]c4)CC3(C)C)oc12 ; CC(C)c1cc(-c2ccc(F)cc2)nc2cc(C(=O)N3CCN(S(=O)(=O)c4ccn(C)n4)CC3(C)C)oc12]

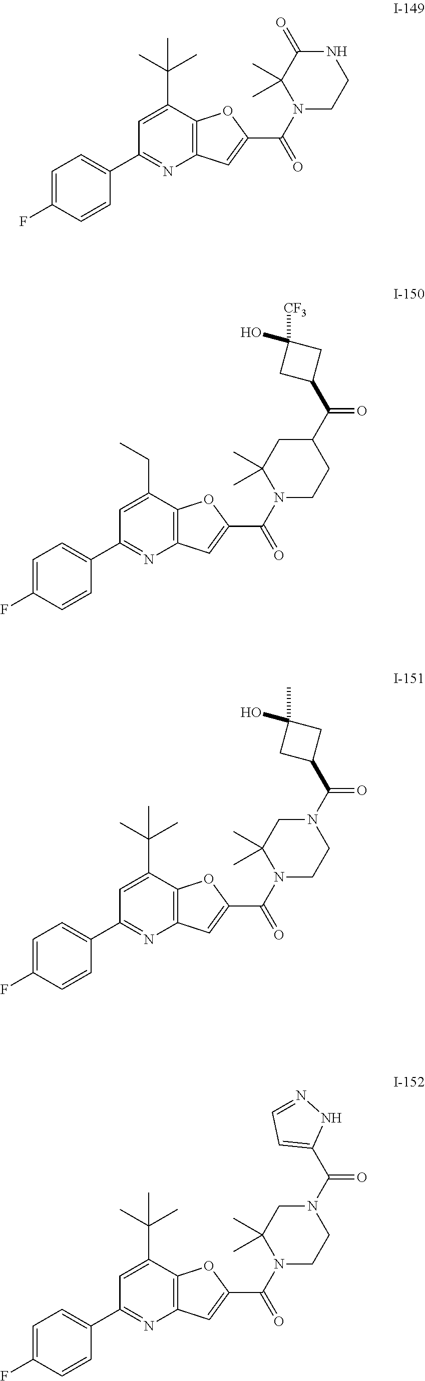

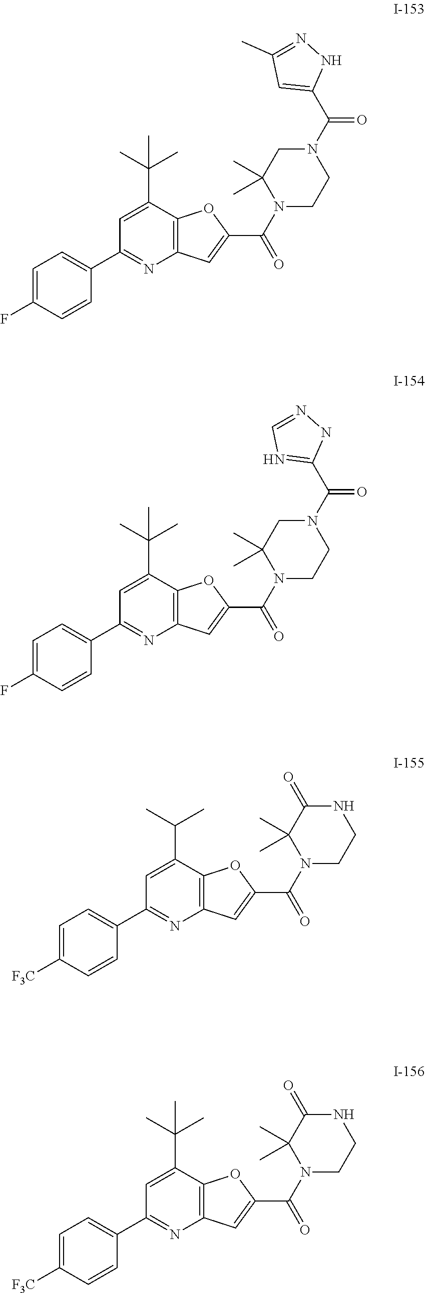 [SMILES: Cc1cc(C(=O)N2CCN(C(=O)c3cc4nc(-c5ccc(F)cc5)cc(C(C)(C)C)c4o3)C(C)(C)C2)[nH]n1 ; CC(C)(C)c1cc(-c2ccc(F)cc2)nc2cc(C(=O)N3CCN(C(=O)c4ncn[nH]4)CC3(C)C)oc12 ; CC(C)c1cc(-c2ccc(C(F)(F)F)cc2)nc2cc(C(=O)N3CCNC(=O)C3(C)C)oc12 ; CC(C)(C)c1cc(-c2ccc(C(F)(F)F)cc2)nc2cc(C(=O)N3CCNC(=O)C3(C)C)oc12]

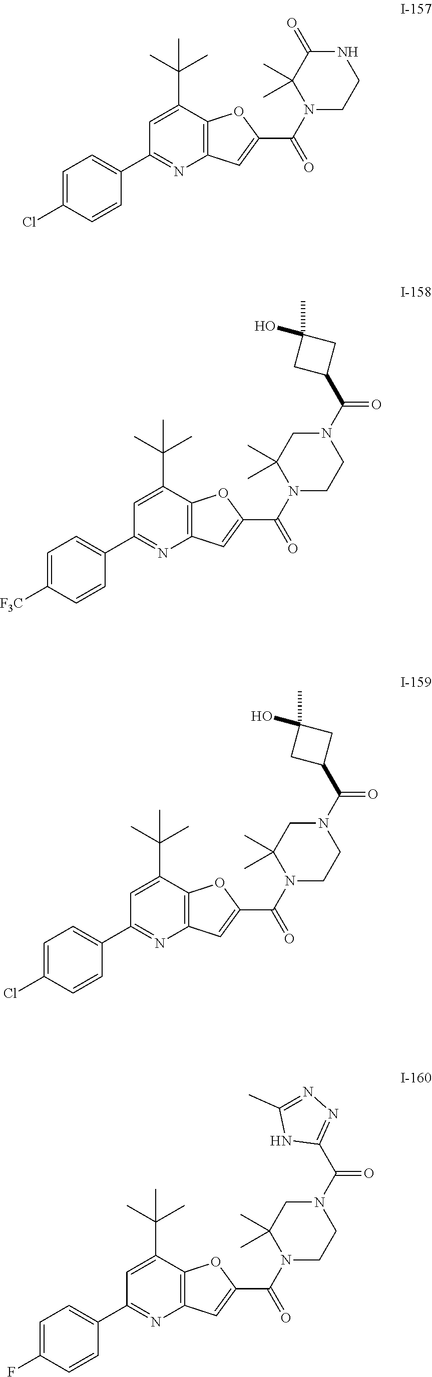

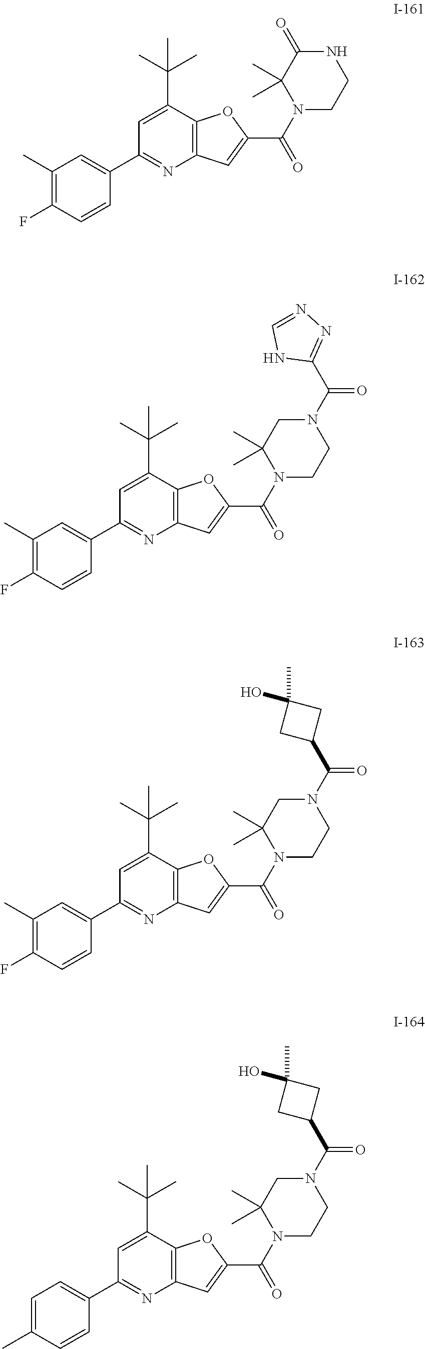

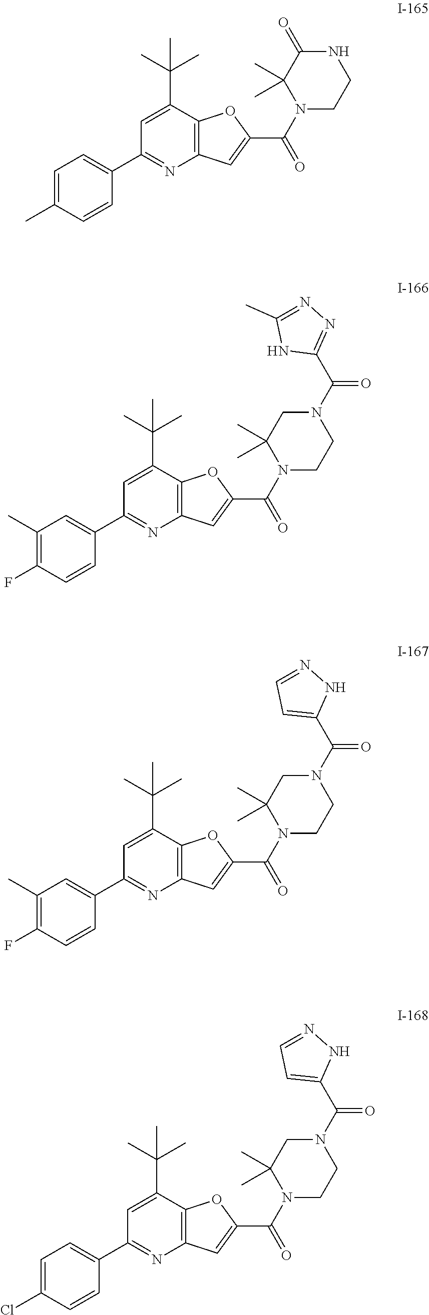 [SMILES: Cc1ccc(-c2cc(C(C)(C)C)c3oc(C(=O)N4CCNC(=O)C4(C)C)cc3n2)cc1 ; Cc1nnc(C(=O)N2CCN(C(=O)c3cc4nc(-c5ccc(F)c(C)c5)cc(C(C)(C)C)c4o3)C(C)(C)C2)[nH]1 ; Cc1cc(-c2cc(C(C)(C)C)c3oc(C(=O)N4CCN(C(=O)c5ccn[nH]5)CC4(C)C)cc3n2)ccc1F ; CC(C)(C)c1cc(-c2ccc(Cl)cc2)nc2cc(C(=O)N3CCN(C(=O)c4ccn[nH]4)CC3(C)C)oc12]

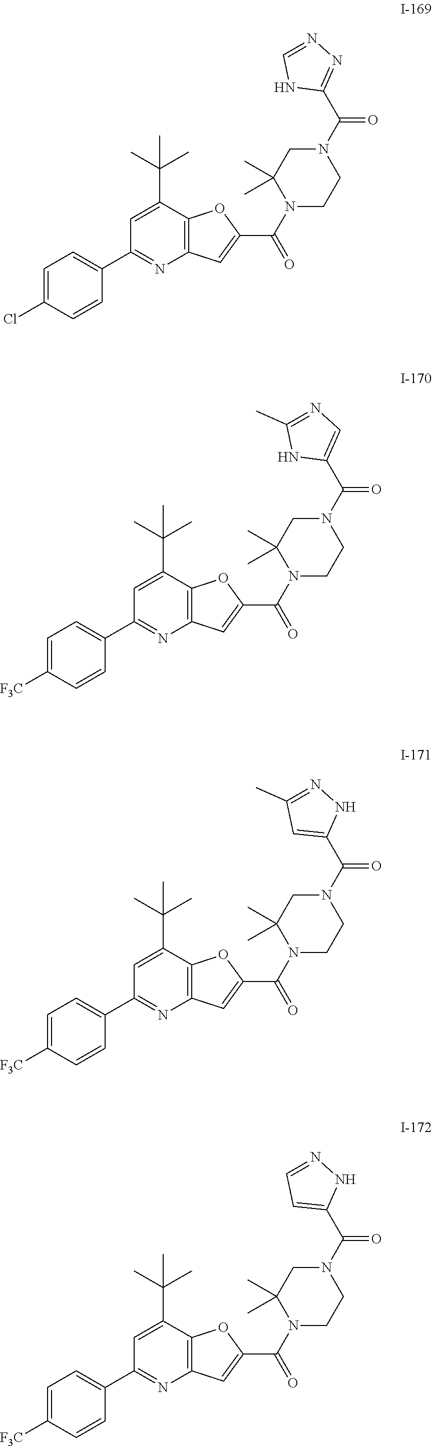 [SMILES: CC(C)(C)c1cc(-c2ccc(Cl)cc2)nc2cc(C(=O)N3CCN(C(=O)c4nnc[nH]4)CC3(C)C)oc12 ; Cc1ncc(C(=O)N2CCN(C(=O)c3cc4nc(-c5ccc(C(F)(F)F)cc5)cc(C(C)(C)C)c4o3)C(C)(C)C2)[nH]1 ; Cc1cc(C(=O)N2CCN(C(=O)c3cc4nc(-c5ccc(C(F)(F)F)cc5)cc(C(C)(C)C)c4o3)C(C)(C)C2)[nH]n1 ; CC(C)(C)c1cc(-c2ccc(C(F)(F)F)cc2)nc2cc(C(=O)N3CCN(C(=O)c4ccn[nH]4)CC3(C)C)oc12]

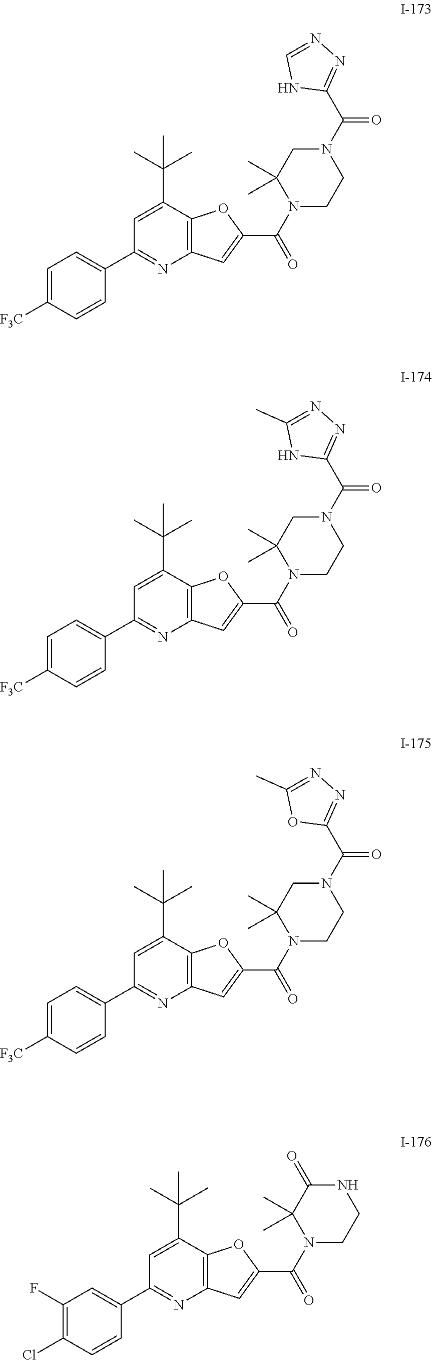 [SMILES: CC(C)(C)c1cc(-c2ccc(C(F)(F)F)cc2)nc2cc(C(=O)N3CCN(C(=O)c4nnc[nH]4)CC3(C)C)oc12 ; Cc1nnc(C(=O)N2CCN(C(=O)c3cc4nc(-c5ccc(C(F)(F)F)cc5)cc(C(C)(C)C)c4o3)C(C)(C)C2)[nH]1 ; Cc1nnc(C(=O)N2CCN(C(=O)c3cc4nc(-c5ccc(C(F)(F)F)cc5)cc(C(C)(C)C)c4o3)C(C)(C)C2)o1 ; CC(C)(C)c1cc(-c2ccc(Cl)c(F)c2)nc2cc(C(=O)N3CCNC(=O)C3(C)C)oc12]

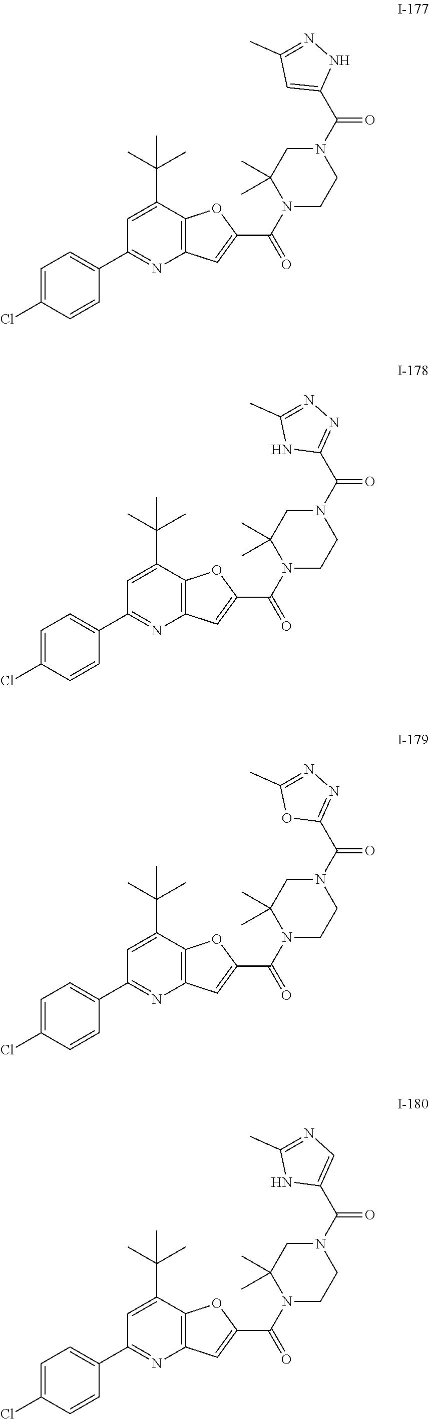 [SMILES: Cc1cc(C(=O)N2CCN(C(=O)c3cc4nc(-c5ccc(Cl)cc5)cc(C(C)(C)C)c4o3)C(C)(C)C2)[nH]n1 ; Cc1nnc(C(=O)N2CCN(C(=O)c3cc4nc(-c5ccc(Cl)cc5)cc(C(C)(C)C)c4o3)C(C)(C)C2)[nH]1 ; Cc1nnc(C(=O)N2CCN(C(=O)c3cc4nc(-c5ccc(Cl)cc5)cc(C(C)(C)C)c4o3)C(C)(C)C2)o1 ; Cc1ncc(C(=O)N2CCN(C(=O)c3cc4nc(-c5ccc(Cl)cc5)cc(C(C)(C)C)c4o3)C(C)(C)C2)[nH]1]

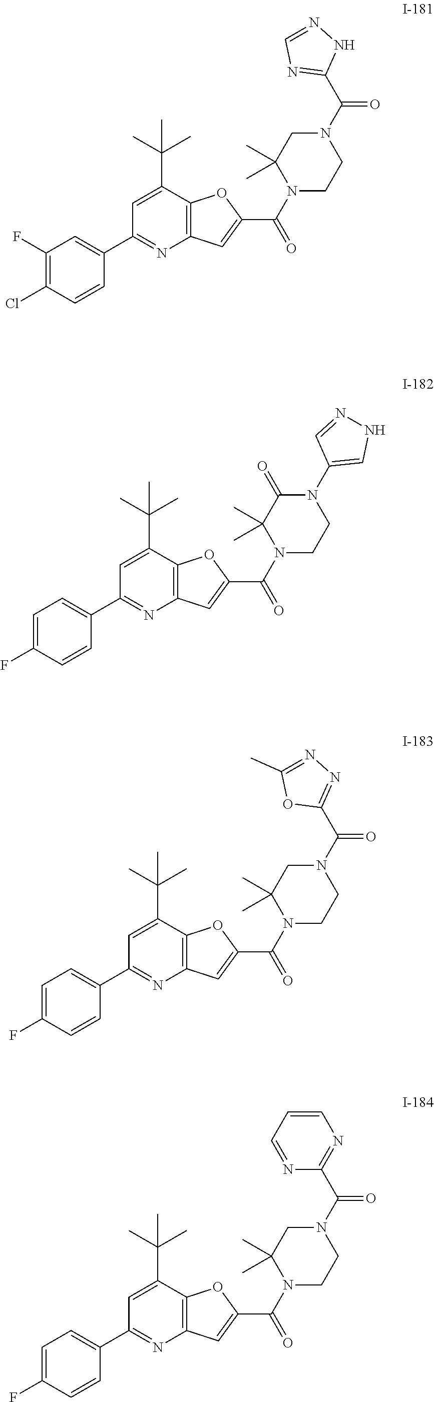 [SMILES: CC(C)(C)c1cc(-c2ccc(Cl)c(F)c2)nc2cc(C(=O)N3CCN(C(=O)c4ncn[nH]4)CC3(C)C)oc12 ; CC(C)(C)c1cc(-c2ccc(F)cc2)nc2cc(C(=O)N3CCN(c4cn[nH]c4)C(=O)C3(C)C)oc12 ; Cc1nnc(C(=O)N2CCN(C(=O)c3cc4nc(-c5ccc(F)cc5)cc(C(C)(C)C)c4o3)C(C)(C)C2)o1 ; CC(C)(C)c1cc(-c2ccc(F)cc2)nc2cc(C(=O)N3CCN(C(=O)c4ncccn4)CC3(C)C)oc12]

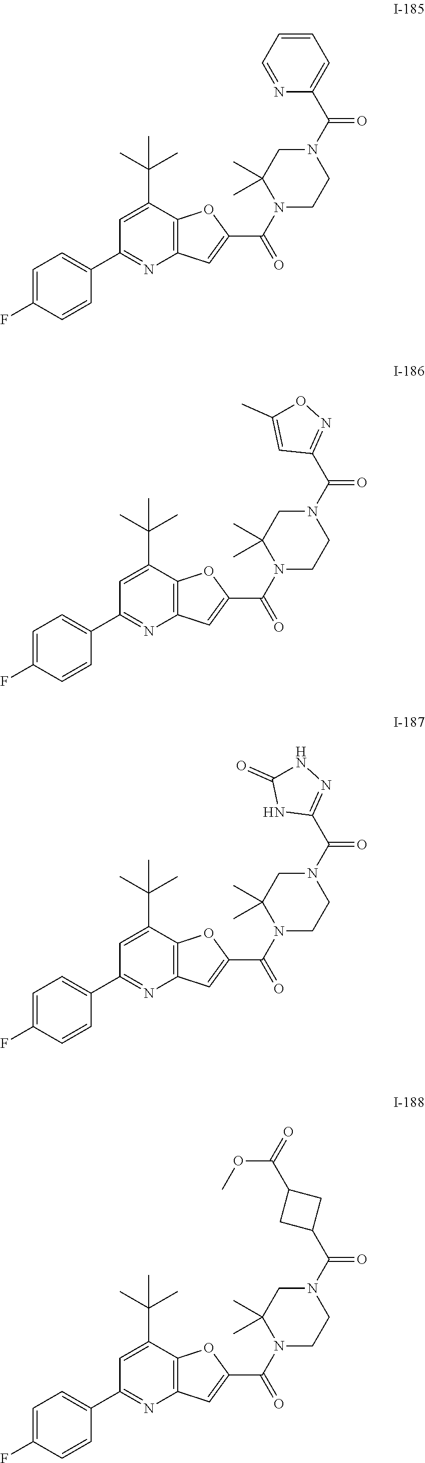 [SMILES: CC(C)(C)c1cc(-c2ccc(F)cc2)nc2cc(C(=O)N3CCN(C(=O)c4ccccn4)CC3(C)C)oc12 ; Cc1cc(C(=O)N2CCN(C(=O)c3cc4nc(-c5ccc(F)cc5)cc(C(C)(C)C)c4o3)C(C)(C)C2)no1 ; CC(C)(C)c1cc(-c2ccc(F)cc2)nc2cc(C(=O)N3CCN(C(=O)c4n[nH]c(=O)[nH]4)CC3(C)C)oc12 ; COC(=O)C1CC(C(=O)N2CCN(C(=O)c3cc4nc(-c5ccc(F)cc5)cc(C(C)(C)C)c4o3)C(C)(C)C2)C1]

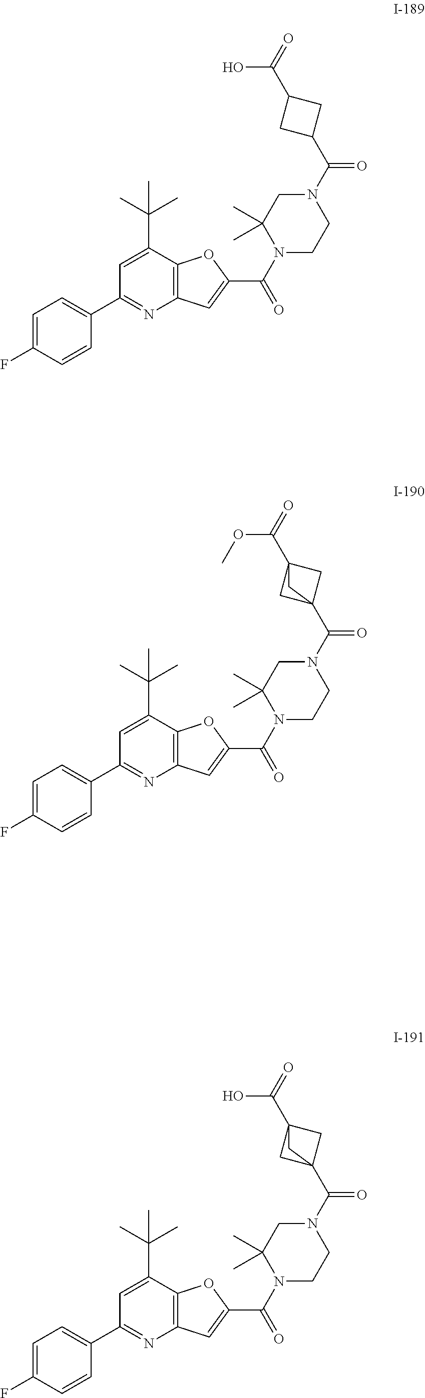 [SMILES: CC(C)(C)c1cc(-c2ccc(F)cc2)nc2cc(C(=O)N3CCN(C(=O)C4CC(C(=O)O)C4)CC3(C)C)oc12 ; COC(=O)C12CC(C(=O)N3CCN(C(=O)c4cc5nc(-c6ccc(F)cc6)cc(C(C)(C)C)c5o4)C(C)(C)C3)(C1)C2 ; CC(C)(C)c1cc(-c2ccc(F)cc2)nc2cc(C(=O)N3CCN(C(=O)C45CC(C(=O)O)(C4)C5)CC3(C)C)oc12]

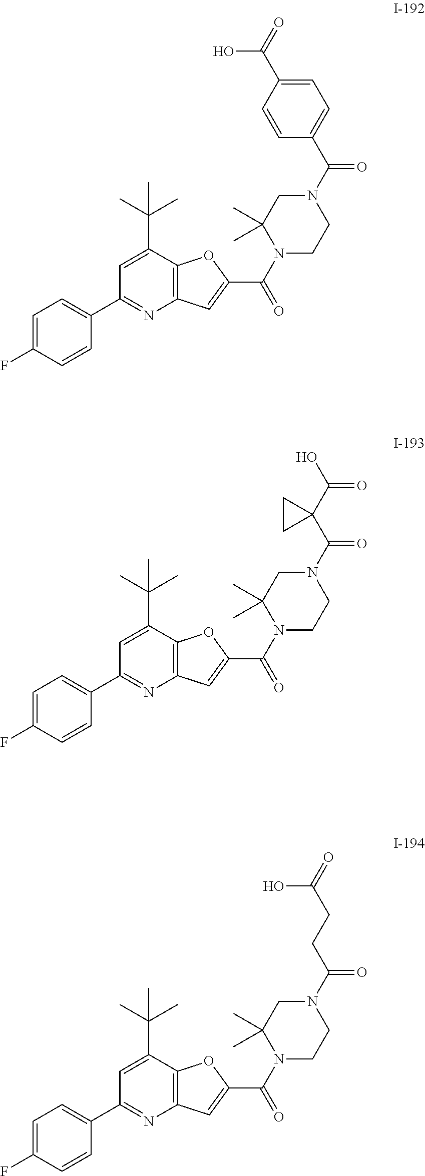 [SMILES: CC(C)(C)c1cc(-c2ccc(F)cc2)nc2cc(C(=O)N3CCN(C(=O)c4ccc(C(=O)O)cc4)CC3(C)C)oc12 ; CC(C)(C)c1cc(-c2ccc(F)cc2)nc2cc(C(=O)N3CCN(C(=O)C4(C(=O)O)CC4)CC3(C)C)oc12 ; CC(C)(C)c1cc(-c2ccc(F)cc2)nc2cc(C(=O)N3CCN(C(=O)CCC(=O)O)CC3(C)C)oc12]

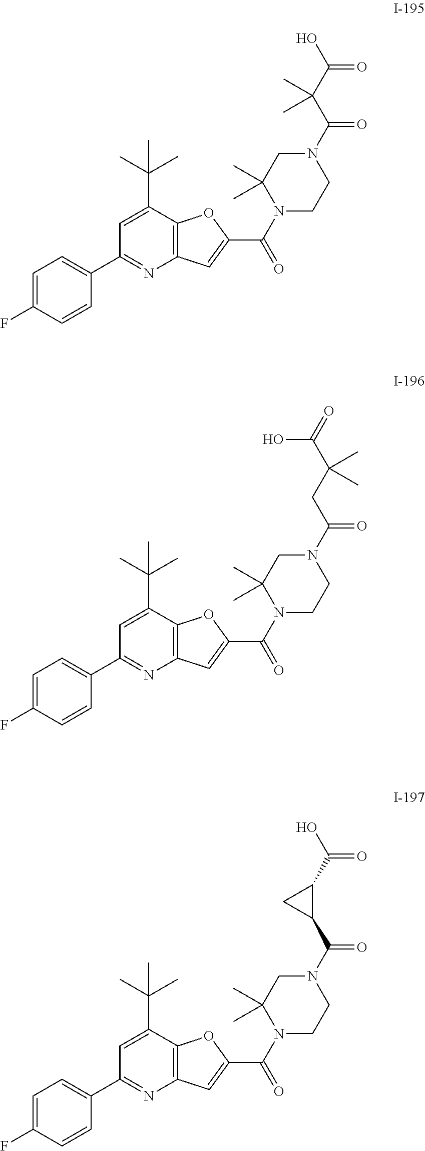 [SMILES: CC(C)(C(=O)O)C(=O)N1CCN(C(=O)c2cc3nc(-c4ccc(F)cc4)cc(C(C)(C)C)c3o2)C(C)(C)C1 ; CC(C)(CC(=O)N1CCN(C(=O)c2cc3nc(-c4ccc(F)cc4)cc(C(C)(C)C)c3o2)C(C)(C)C1)C(=O)O ; CC(C)(C)c1cc(-c2ccc(F)cc2)nc2cc(C(=O)N3CCN(C(=O)[C@H]4C[C@@H]4C(=O)O)CC3(C)C)oc12]

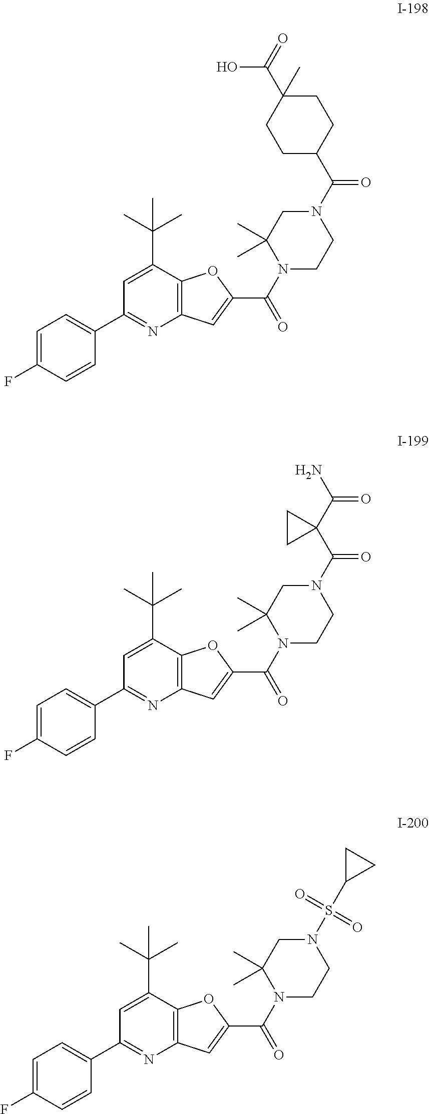 [SMILES: CC1(C(=O)O)CCC(C(=O)N2CCN(C(=O)c3cc4nc(-c5ccc(F)cc5)cc(C(C)(C)C)c4o3)C(C)(C)C2)CC1 ; CC(C)(C)c1cc(-c2ccc(F)cc2)nc2cc(C(=O)N3CCN(C(=O)C4(C(N)=O)CC4)CC3(C)C)oc12 ; CC(C)(C)c1cc(-c2ccc(F)cc2)nc2cc(C(=O)N3CCN(S(=O)(=O)C4CC4)CC3(C)C)oc12]

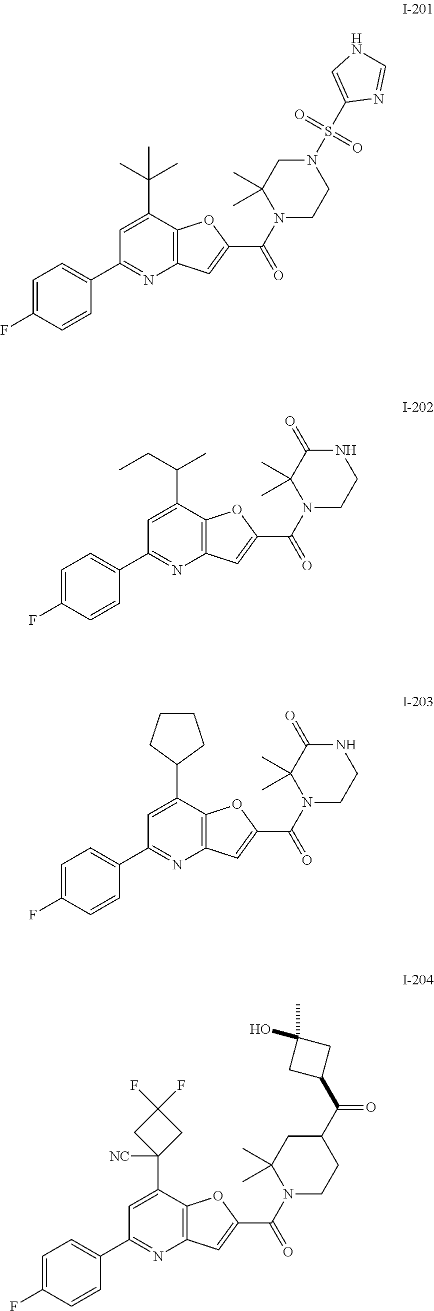

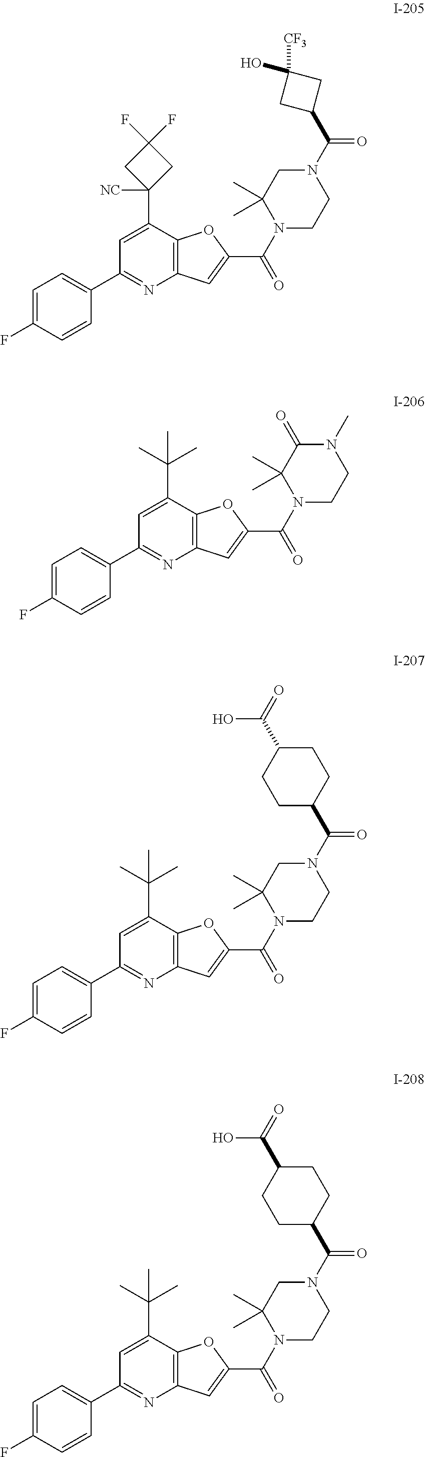

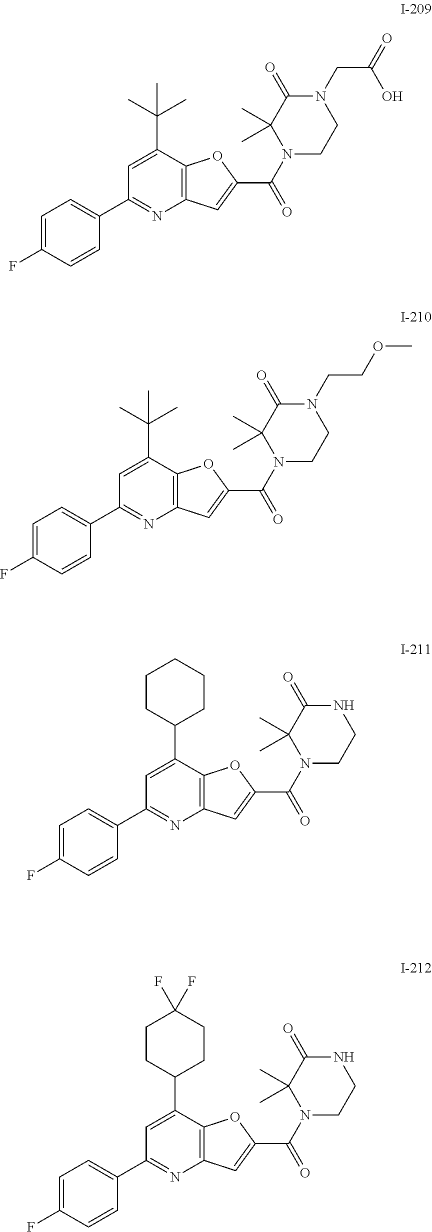 [SMILES: CC(C)(C)c1cc(-c2ccc(F)cc2)nc2cc(C(=O)N3CCN(CC(=O)O)C(=O)C3(C)C)oc12 ; COCCN1CCN(C(=O)c2cc3nc(-c4ccc(F)cc4)cc(C(C)(C)C)c3o2)C(C)(C)C1=O ; CC1(C)C(=O)NCCN1C(=O)c1cc2nc(-c3ccc(F)cc3)cc(C3CCCCC3)c2o1 ; CC1(C)C(=O)NCCN1C(=O)c1cc2nc(-c3ccc(F)cc3)cc(C3CCC(F)(F)CC3)c2o1]

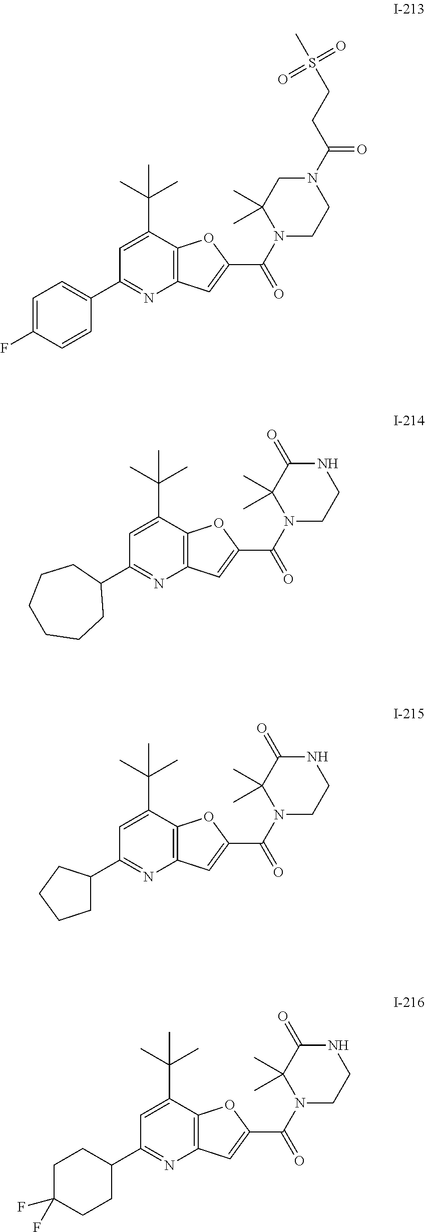 [SMILES: CC(C)(C)c1cc(-c2ccc(F)cc2)nc2cc(C(=O)N3CCN(C(=O)CCS(C)(=O)=O)CC3(C)C)oc12 ; CC(C)(C)c1cc(C2CCCCCC2)nc2cc(C(=O)N3CCNC(=O)C3(C)C)oc12 ; CC(C)(C)c1cc(C2CCCC2)nc2cc(C(=O)N3CCNC(=O)C3(C)C)oc12 ; CC(C)(C)c1cc(C2CCC(F)(F)CC2)nc2cc(C(=O)N3CCNC(=O)C3(C)C)oc12]

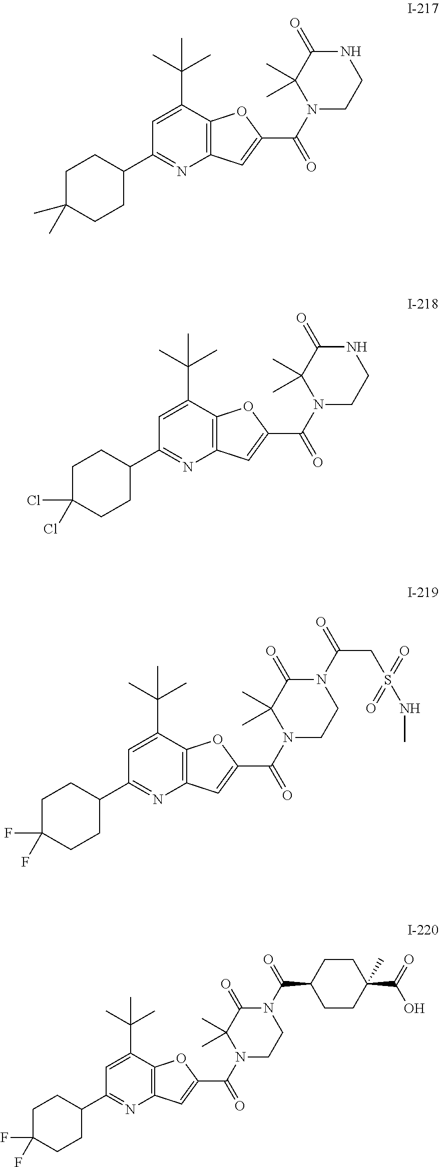 [SMILES: CC1(C)CCC(c2cc(C(C)(C)C)c3oc(C(=O)N4CCNC(=O)C4(C)C)cc3n2)CC1 ; CC(C)(C)c1cc(C2CCC(Cl)(Cl)CC2)nc2cc(C(=O)N3CCNC(=O)C3(C)C)oc12 ; CNS(=O)(=O)CC(=O)N1CCN(C(=O)c2cc3nc(C4CCC(F)(F)CC4)cc(C(C)(C)C)c3o2)C(C)(C)C1=O ; CC(C)(C)c1cc(C2CCC(F)(F)CC2)nc2cc(C(=O)N3CCN(C(=O)[C@H]4CC[C@@](C)(C(=O)O)CC4)C(=O)C3(C)C)oc12]

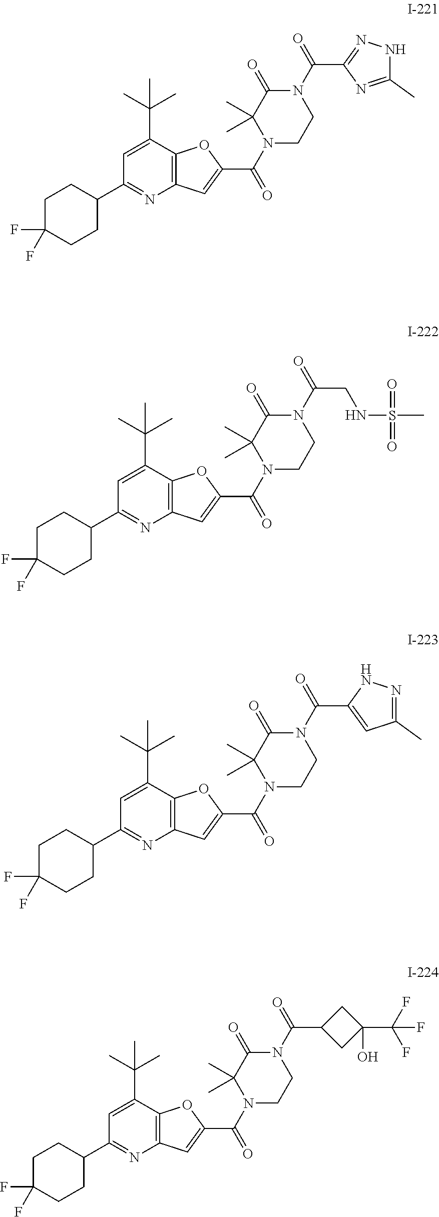 [SMILES: Cc1nc(C(=O)N2CCN(C(=O)c3cc4nc(C5CCC(F)(F)CC5)cc(C(C)(C)C)c4o3)C(C)(C)C2=O)n[nH]1 ; CC(C)(C)c1cc(C2CCC(F)(F)CC2)nc2cc(C(=O)N3CCN(C(=O)CNS(C)(=O)=O)C(=O)C3(C)C)oc12 ; Cc1cc(C(=O)N2CCN(C(=O)c3cc4nc(C5CCC(F)(F)CC5)cc(C(C)(C)C)c4o3)C(C)(C)C2=O)[nH]n1 ; CC(C)(C)c1cc(C2CCC(F)(F)CC2)nc2cc(C(=O)N3CCN(C(=O)C4CC(O)(C(F)(F)F)C4)C(=O)C3(C)C)oc12]

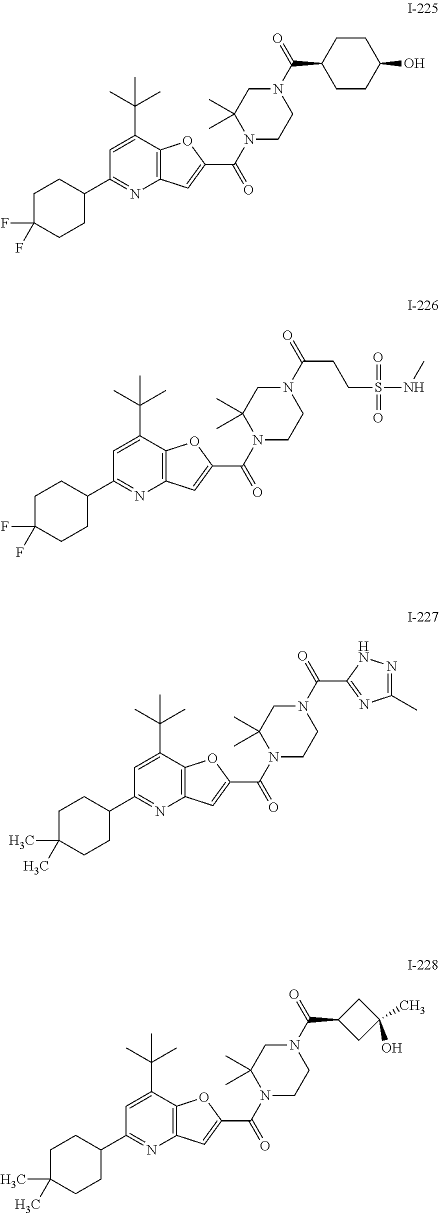

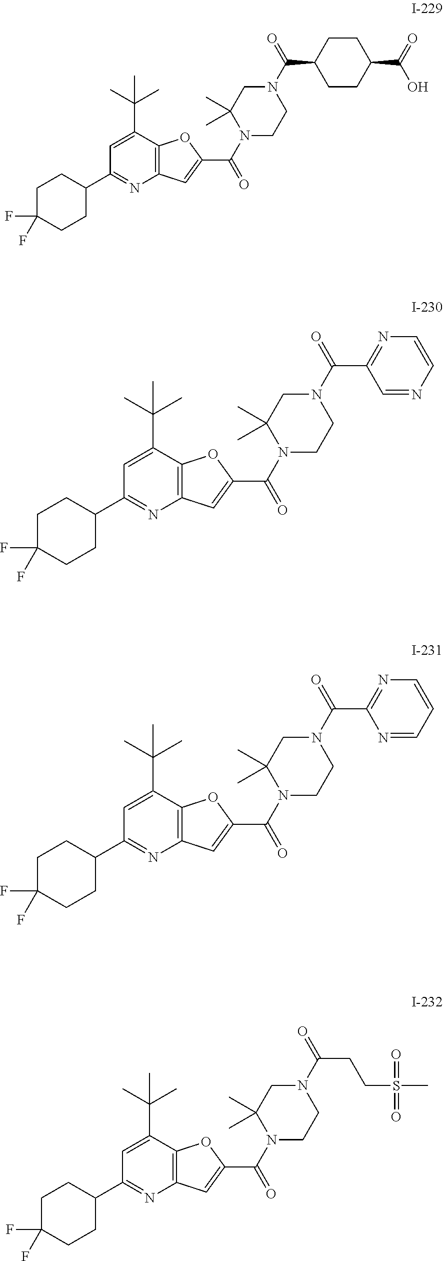 [SMILES: CC(C)(C)c1cc(C2CCC(F)(F)CC2)nc2cc(C(=O)N3CCN(C(=O)[C@H]4CC[C@@H](C(=O)O)CC4)CC3(C)C)oc12 ; CC(C)(C)c1cc(C2CCC(F)(F)CC2)nc2cc(C(=O)N3CCN(C(=O)c4cnccn4)CC3(C)C)oc12 ; CC(C)(C)c1cc(C2CCC(F)(F)CC2)nc2cc(C(=O)N3CCN(C(=O)c4ncccn4)CC3(C)C)oc12 ; CC(C)(C)c1cc(C2CCC(F)(F)CC2)nc2cc(C(=O)N3CCN(C(=O)CCS(C)(=O)=O)CC3(C)C)oc12]

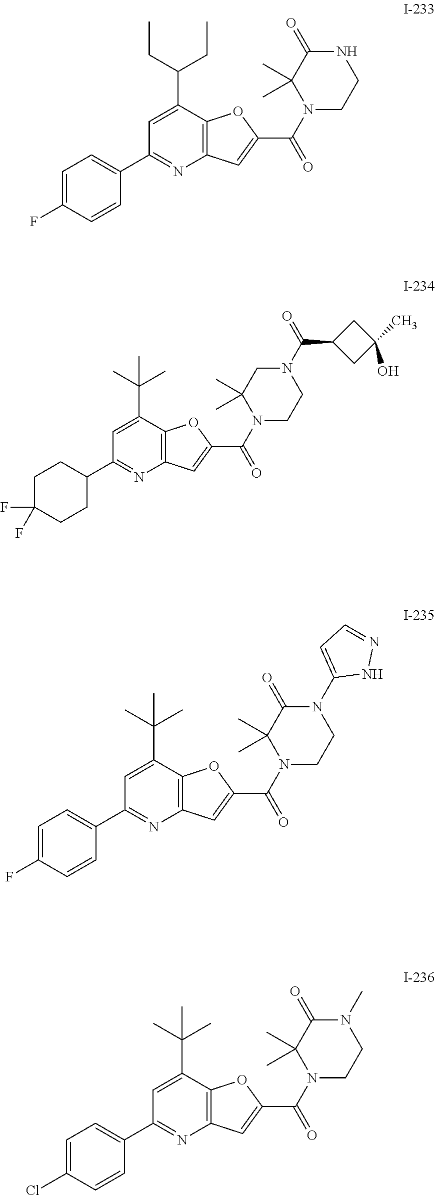 [SMILES: CCC(CC)c1cc(-c2ccc(F)cc2)nc2cc(C(=O)N3CCNC(=O)C3(C)C)oc12 ; CC(C)(C)c1cc(C2CCC(F)(F)CC2)nc2cc(C(=O)N3CCN(C(=O)[C@H]4C[C@@](C)(O)C4)CC3(C)C)oc12 ; CC(C)(C)c1cc(-c2ccc(F)cc2)nc2cc(C(=O)N3CCN(c4ccn[nH]4)C(=O)C3(C)C)oc12 ; CN1CCN(C(=O)c2cc3nc(-c4ccc(Cl)cc4)cc(C(C)(C)C)c3o2)C(C)(C)C1=O]

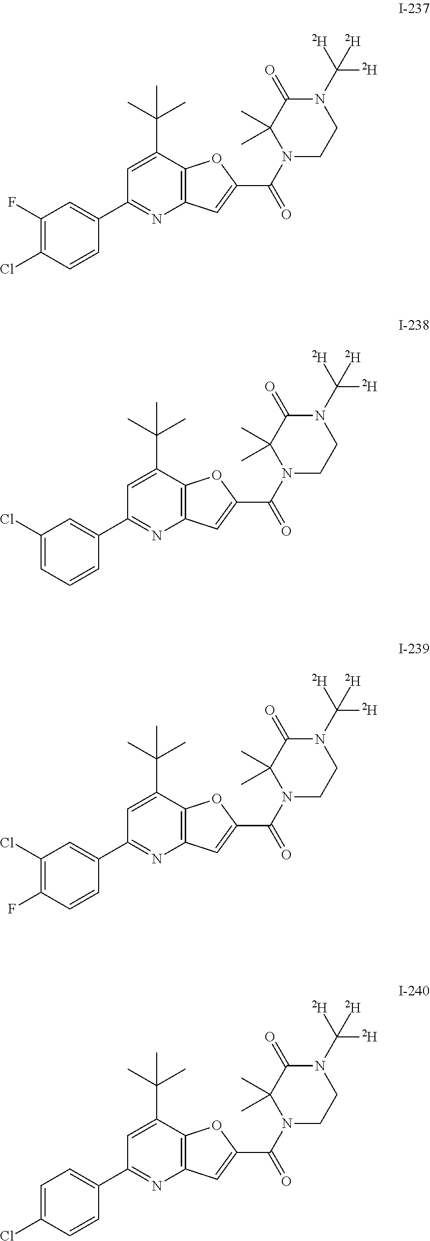 [SMILES: [2H]C([2H])([2H])N1CCN(C(=O)c2cc3nc(-c4ccc(Cl)c(F)c4)cc(C(C)(C)C)c3o2)C(C)(C)C1=O ; [2H]C([2H])([2H])N1CCN(C(=O)c2cc3nc(-c4cccc(Cl)c4)cc(C(C)(C)C)c3o2)C(C)(C)C1=O ; [2H]C([2H])([2H])N1CCN(C(=O)c2cc3nc(-c4ccc(F)c(Cl)c4)cc(C(C)(C)C)c3o2)C(C)(C)C1=O ; [2H]C([2H])([2H])N1CCN(C(=O)c2cc3nc(-c4ccc(Cl)cc4)cc(C(C)(C)C)c3o2)C(C)(C)C1=O]

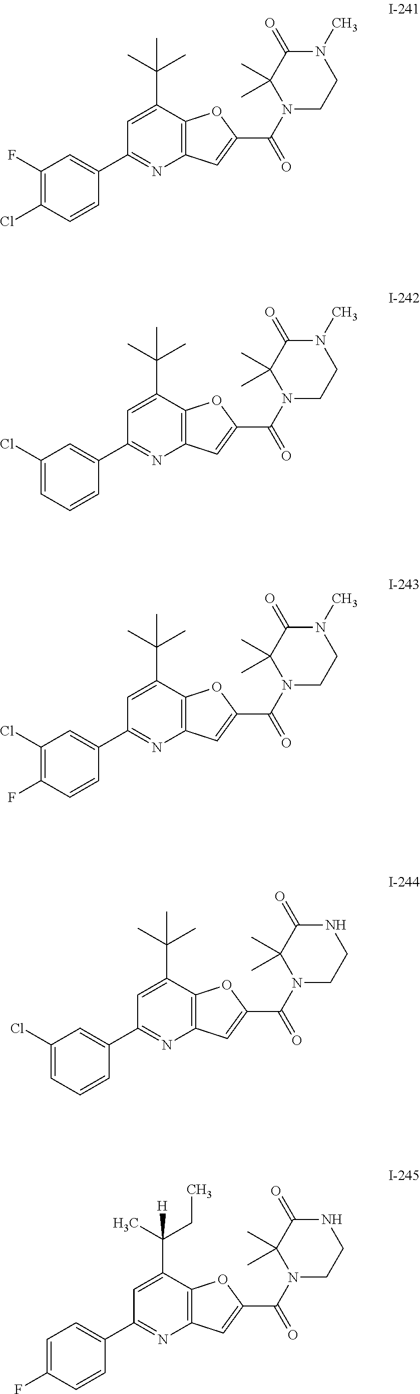 [SMILES: CN1CCN(C(=O)c2cc3nc(-c4ccc(Cl)c(F)c4)cc(C(C)(C)C)c3o2)C(C)(C)C1=O ; CN1CCN(C(=O)c2cc3nc(-c4cccc(Cl)c4)cc(C(C)(C)C)c3o2)C(C)(C)C1=O ; CN1CCN(C(=O)c2cc3nc(-c4ccc(F)c(Cl)c4)cc(C(C)(C)C)c3o2)C(C)(C)C1=O ; CC(C)(C)c1cc(-c2cccc(Cl)c2)nc2cc(C(=O)N3CCNC(=O)C3(C)C)oc12 ; CC[C@@H](C)c1cc(-c2ccc(F)cc2)nc2cc(C(=O)N3CCNC(=O)C3(C)C)oc12]

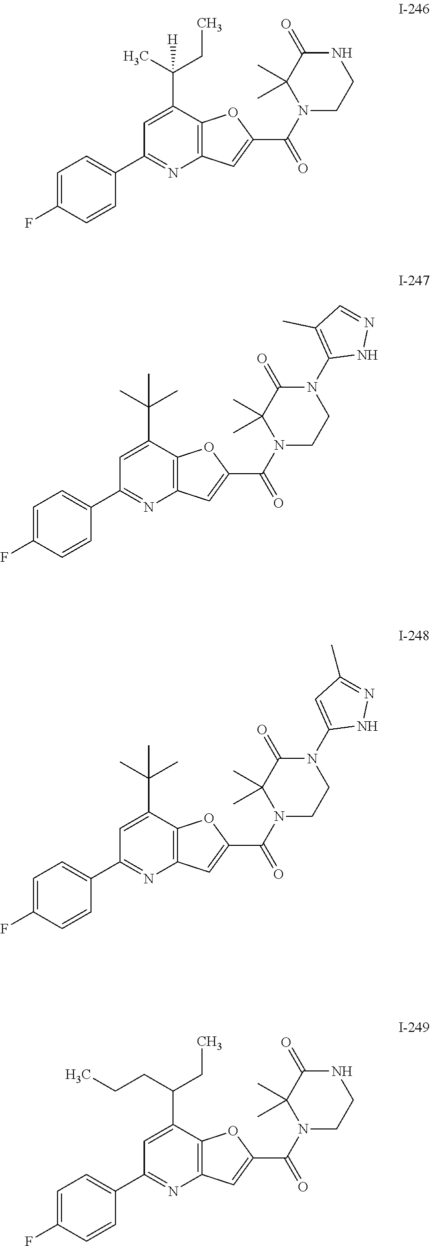 [SMILES: CC[C@H](C)c1cc(-c2ccc(F)cc2)nc2cc(C(=O)N3CCNC(=O)C3(C)C)oc12 ; Cc1cn[nH]c1N1CCN(C(=O)c2cc3nc(-c4ccc(F)cc4)cc(C(C)(C)C)c3o2)C(C)(C)C1=O ; Cc1cc(N2CCN(C(=O)c3cc4nc(-c5ccc(F)cc5)cc(C(C)(C)C)c4o3)C(C)(C)C2=O)[nH]n1 ; CCCC(CC)c1cc(-c2ccc(F)cc2)nc2cc(C(=O)N3CCNC(=O)C3(C)C)oc12]

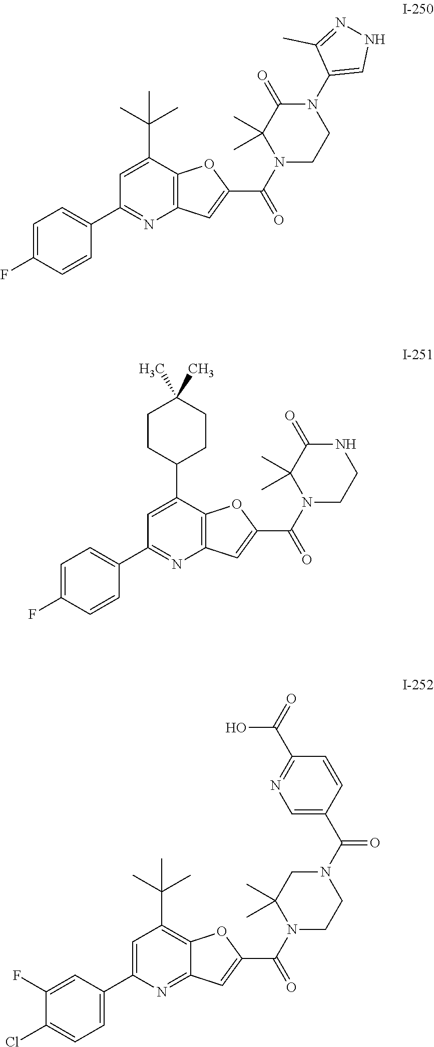

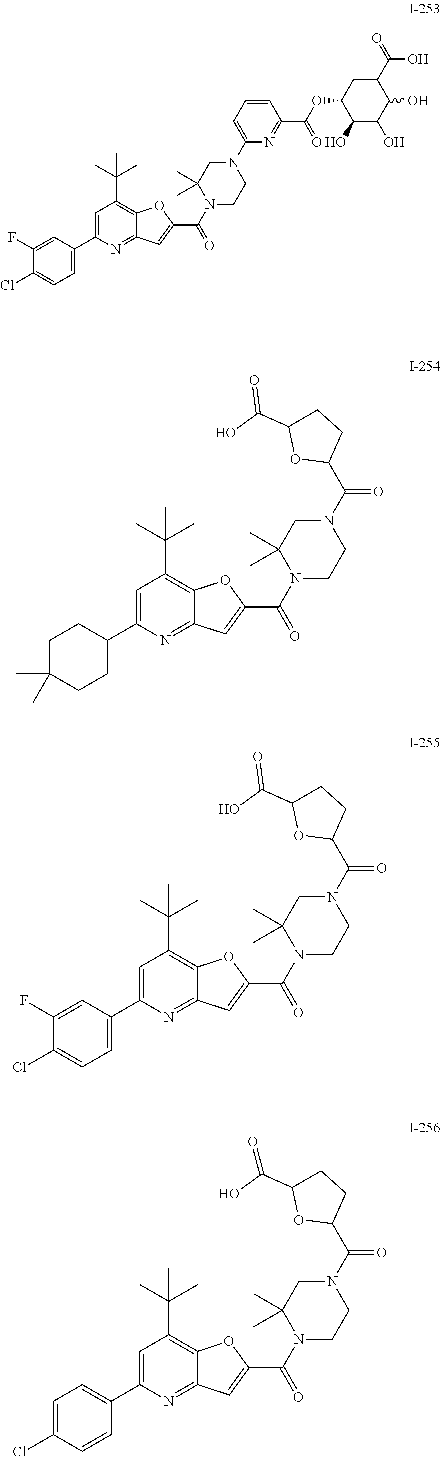 [SMILES: CC(C)(C)c1cc(-c2ccc(Cl)c(F)c2)nc2cc(C(=O)N3CCN(c4cccc(C(=O)O[C@@H]5CC(C(=O)O)C(O)C(O)[C@H]5O)n4)CC3(C)C)oc12 ; CC1(C)CCC(c2cc(C(C)(C)C)c3oc(C(=O)N4CCN(C(=O)C5CCC(C(=O)O)O5)CC4(C)C)cc3n2)CC1 ; CC(C)(C)c1cc(-c2ccc(Cl)c(F)c2)nc2cc(C(=O)N3CCN(C(=O)C4CCC(C(=O)O)O4)CC3(C)C)oc12 ; CC(C)(C)c1cc(-c2ccc(Cl)cc2)nc2cc(C(=O)N3CCN(C(=O)C4CCC(C(=O)O)O4)CC3(C)C)oc12]

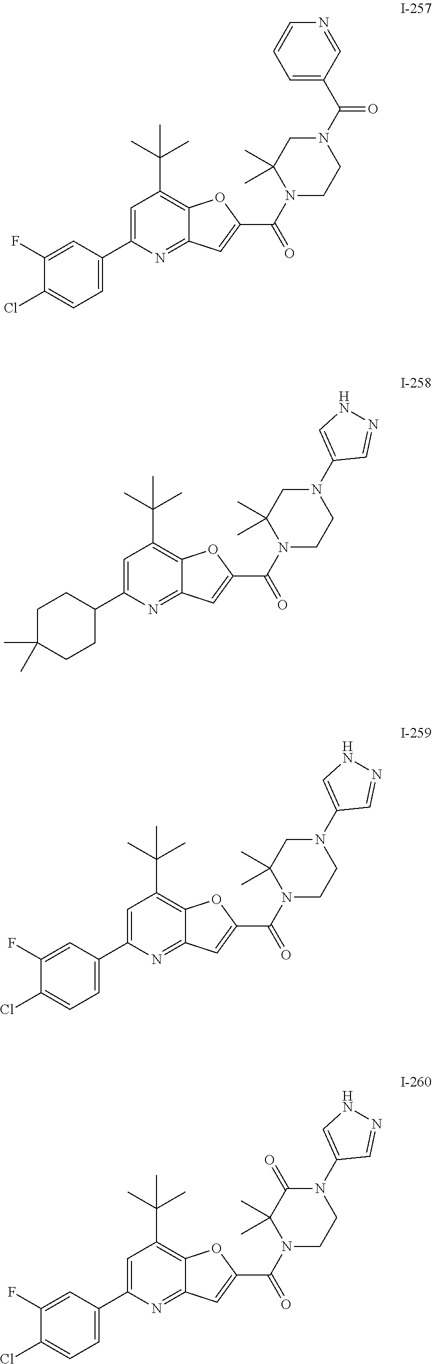 [SMILES: CC(C)(C)c1cc(-c2ccc(Cl)c(F)c2)nc2cc(C(=O)N3CCN(C(=O)c4cccnc4)CC3(C)C)oc12 ; CC1(C)CCC(c2cc(C(C)(C)C)c3oc(C(=O)N4CCN(c5cn[nH]c5)CC4(C)C)cc3n2)CC1 ; CC(C)(C)c1cc(-c2ccc(Cl)c(F)c2)nc2cc(C(=O)N3CCN(c4cn[nH]c4)CC3(C)C)oc12 ; CC(C)(C)c1cc(-c2ccc(Cl)c(F)c2)nc2cc(C(=O)N3CCN(c4cn[nH]c4)C(=O)C3(C)C)oc12]

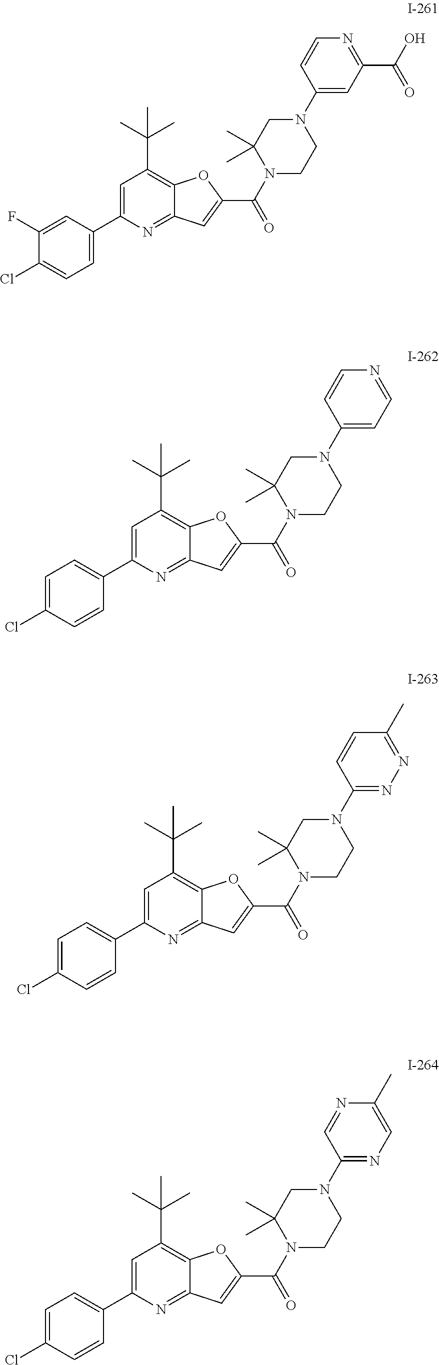 [SMILES: CC(C)(C)c1cc(-c2ccc(Cl)c(F)c2)nc2cc(C(=O)N3CCN(c4ccnc(C(=O)O)c4)CC3(C)C)oc12 ; CC(C)(C)c1cc(-c2ccc(Cl)cc2)nc2cc(C(=O)N3CCN(c4ccncc4)CC3(C)C)oc12 ; Cc1ccc(N2CCN(C(=O)c3cc4nc(-c5ccc(Cl)cc5)cc(C(C)(C)C)c4o3)C(C)(C)C2)nn1 ; Cc1cnc(N2CCN(C(=O)c3cc4nc(-c5ccc(Cl)cc5)cc(C(C)(C)C)c4o3)C(C)(C)C2)cn1]

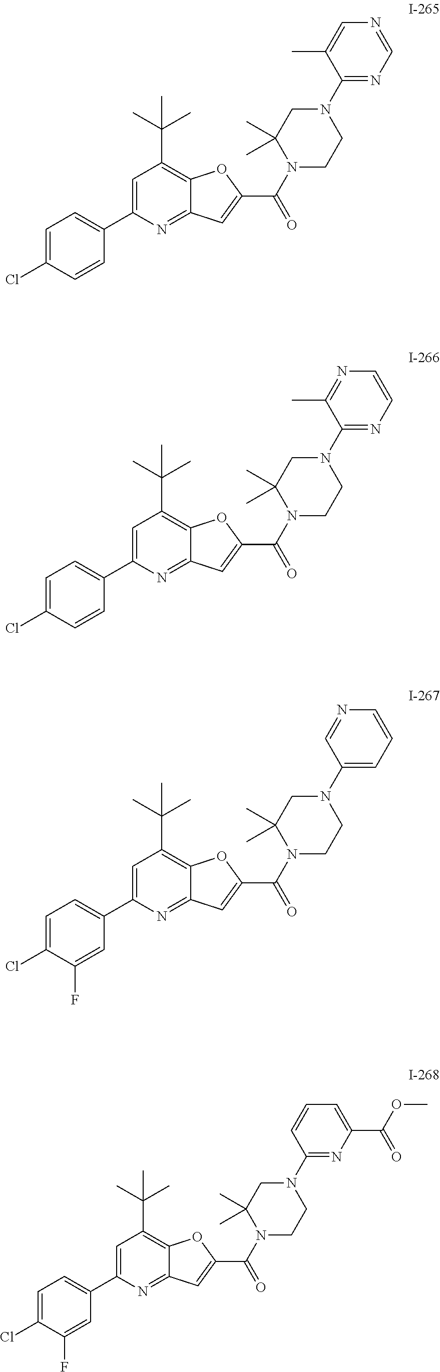 [SMILES: Cc1cncnc1N1CCN(C(=O)c2cc3nc(-c4ccc(Cl)cc4)cc(C(C)(C)C)c3o2)C(C)(C)C1 ; Cc1nccnc1N1CCN(C(=O)c2cc3nc(-c4ccc(Cl)cc4)cc(C(C)(C)C)c3o2)C(C)(C)C1 ; CC(C)(C)c1cc(-c2ccc(Cl)c(F)c2)nc2cc(C(=O)N3CCN(c4cccnc4)CC3(C)C)oc12 ; COC(=O)c1cccc(N2CCN(C(=O)c3cc4nc(-c5ccc(Cl)c(F)c5)cc(C(C)(C)C)c4o3)C(C)(C)C2)n1]

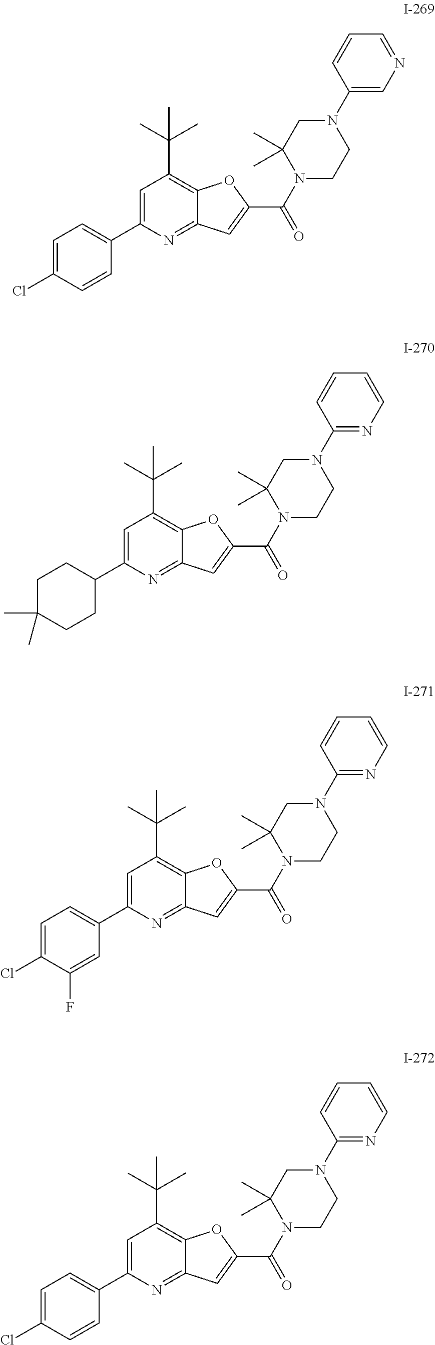 [SMILES: CC(C)(C)c1cc(-c2ccc(Cl)cc2)nc2cc(C(=O)N3CCN(c4cccnc4)CC3(C)C)oc12 ; CC1(C)CCC(c2cc(C(C)(C)C)c3oc(C(=O)N4CCN(c5ccccn5)CC4(C)C)cc3n2)CC1 ; CC(C)(C)c1cc(-c2ccc(Cl)c(F)c2)nc2cc(C(=O)N3CCN(c4ccccn4)CC3(C)C)oc12 ; CC(C)(C)c1cc(-c2ccc(Cl)cc2)nc2cc(C(=O)N3CCN(c4ccccn4)CC3(C)C)oc12]

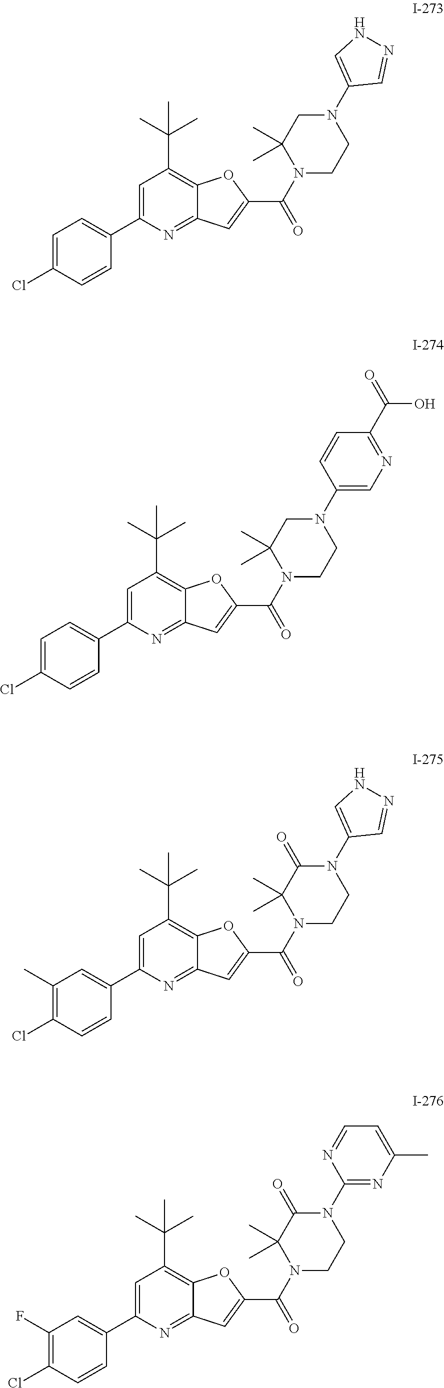 [SMILES: CC(C)(C)c1cc(-c2ccc(Cl)cc2)nc2cc(C(=O)N3CCN(c4cn[nH]c4)CC3(C)C)oc12 ; CC(C)(C)c1cc(-c2ccc(Cl)cc2)nc2cc(C(=O)N3CCN(c4ccc(C(=O)O)nc4)CC3(C)C)oc12 ; Cc1cc(-c2cc(C(C)(C)C)c3oc(C(=O)N4CCN(c5cn[nH]c5)C(=O)C4(C)C)cc3n2)ccc1Cl ; Cc1ccnc(N2CCN(C(=O)c3cc4nc(-c5ccc(Cl)c(F)c5)cc(C(C)(C)C)c4o3)C(C)(C)C2=O)n1]

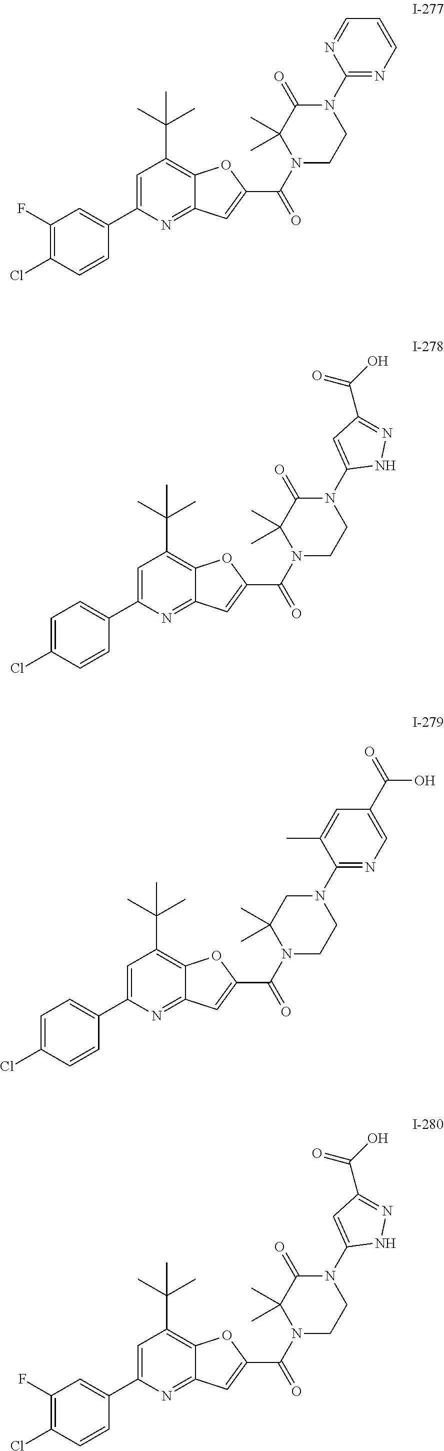 [SMILES: CC(C)(C)c1cc(-c2ccc(Cl)c(F)c2)nc2cc(C(=O)N3CCN(c4ncccn4)C(=O)C3(C)C)oc12 ; CC(C)(C)c1cc(-c2ccc(Cl)cc2)nc2cc(C(=O)N3CCN(c4cc(C(=O)O)n[nH]4)C(=O)C3(C)C)oc12 ; Cc1cc(C(=O)O)cnc1N1CCN(C(=O)c2cc3nc(-c4ccc(Cl)cc4)cc(C(C)(C)C)c3o2)C(C)(C)C1 ; CC(C)(C)c1cc(-c2ccc(Cl)c(F)c2)nc2cc(C(=O)N3CCN(c4cc(C(=O)O)n[nH]4)C(=O)C3(C)C)oc12]

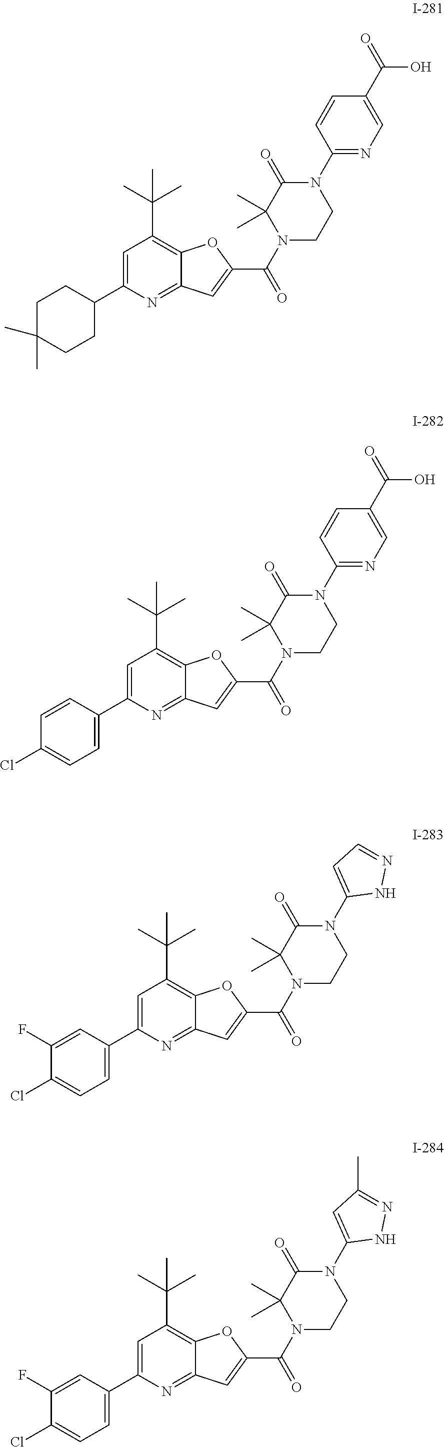 [SMILES: CC1(C)CCC(c2cc(C(C)(C)C)c3oc(C(=O)N4CCN(c5ccc(C(=O)O)cn5)C(=O)C4(C)C)cc3n2)CC1 ; CC(C)(C)c1cc(-c2ccc(Cl)cc2)nc2cc(C(=O)N3CCN(c4ccc(C(=O)O)cn4)C(=O)C3(C)C)oc12 ; CC(C)(C)c1cc(-c2ccc(Cl)c(F)c2)nc2cc(C(=O)N3CCN(c4ccn[nH]4)C(=O)C3(C)C)oc12 ; Cc1cc(N2CCN(C(=O)c3cc4nc(-c5ccc(Cl)c(F)c5)cc(C(C)(C)C)c4o3)C(C)(C)C2=O)[nH]n1]

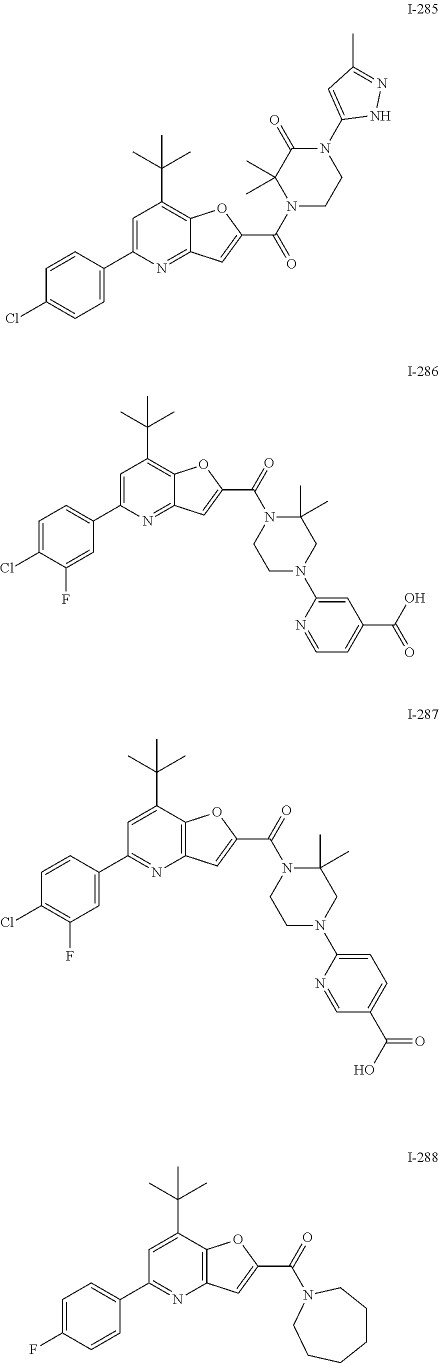 [SMILES: Cc1cc(N2CCN(C(=O)c3cc4nc(-c5ccc(Cl)cc5)cc(C(C)(C)C)c4o3)C(C)(C)C2=O)[nH]n1 ; CC(C)(C)c1cc(-c2ccc(Cl)c(F)c2)nc2cc(C(=O)N3CCN(c4cc(C(=O)O)ccn4)CC3(C)C)oc12 ; CC(C)(C)c1cc(-c2ccc(Cl)c(F)c2)nc2cc(C(=O)N3CCN(c4ccc(C(=O)O)cn4)CC3(C)C)oc12 ; CC(C)(C)c1cc(-c2ccc(F)cc2)nc2cc(C(=O)N3CCCCCC3)oc12]

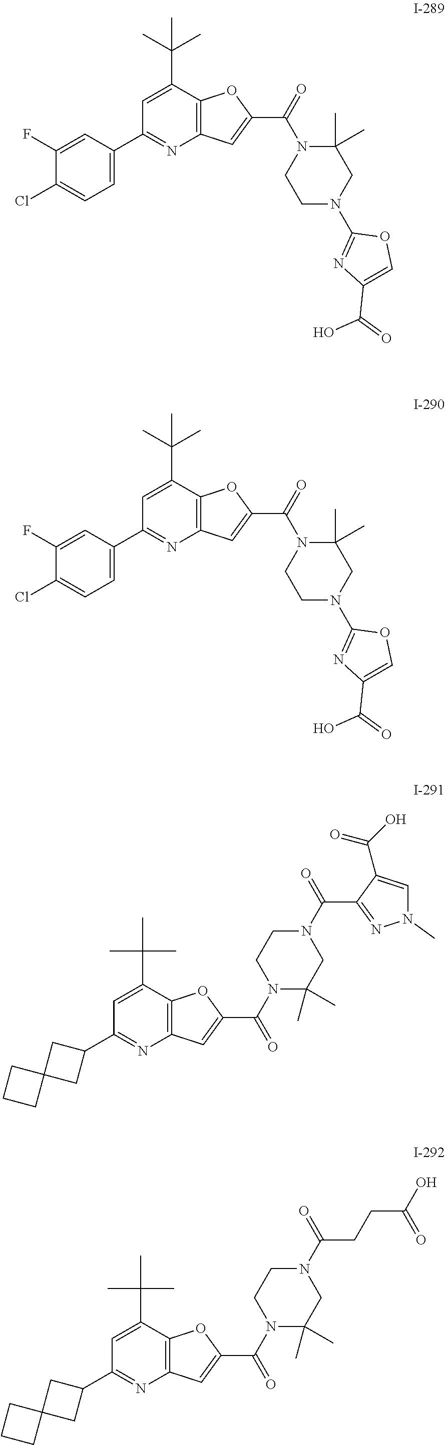 [SMILES: CC(C)(C)c1cc(-c2ccc(Cl)c(F)c2)nc2cc(C(=O)N3CCN(c4nc(C(=O)O)co4)CC3(C)C)oc12 ; CC(C)(C)c1cc(-c2ccc(Cl)c(F)c2)nc2cc(C(=O)N3CCN(c4nc(C(=O)O)co4)CC3(C)C)oc12 ; Cn1cc(C(=O)O)c(C(=O)N2CCN(C(=O)c3cc4nc(C5CC6(CCC6)C5)cc(C(C)(C)C)c4o3)C(C)(C)C2)n1 ; CC(C)(C)c1cc(C2CC3(CCC3)C2)nc2cc(C(=O)N3CCN(C(=O)CCC(=O)O)CC3(C)C)oc12]

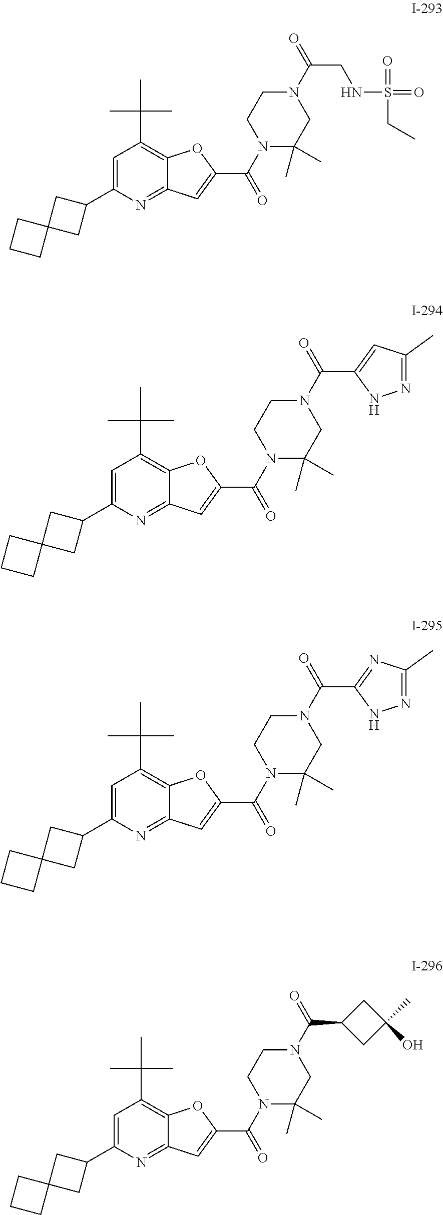 [SMILES: CCS(=O)(=O)NCC(=O)N1CCN(C(=O)c2cc3nc(C4CC5(CCC5)C4)cc(C(C)(C)C)c3o2)C(C)(C)C1 ; Cc1cc(C(=O)N2CCN(C(=O)c3cc4nc(C5CC6(CCC6)C5)cc(C(C)(C)C)c4o3)C(C)(C)C2)[nH]n1 ; Cc1n[nH]c(C(=O)N2CCN(C(=O)c3cc4nc(C5CC6(CCC6)C5)cc(C(C)(C)C)c4o3)C(C)(C)C2)n1 ; CC(C)(C)c1cc(C2CC3(CCC3)C2)nc2cc(C(=O)N3CCN(C(=O)[C@H]4C[C@@](C)(O)C4)CC3(C)C)oc12]

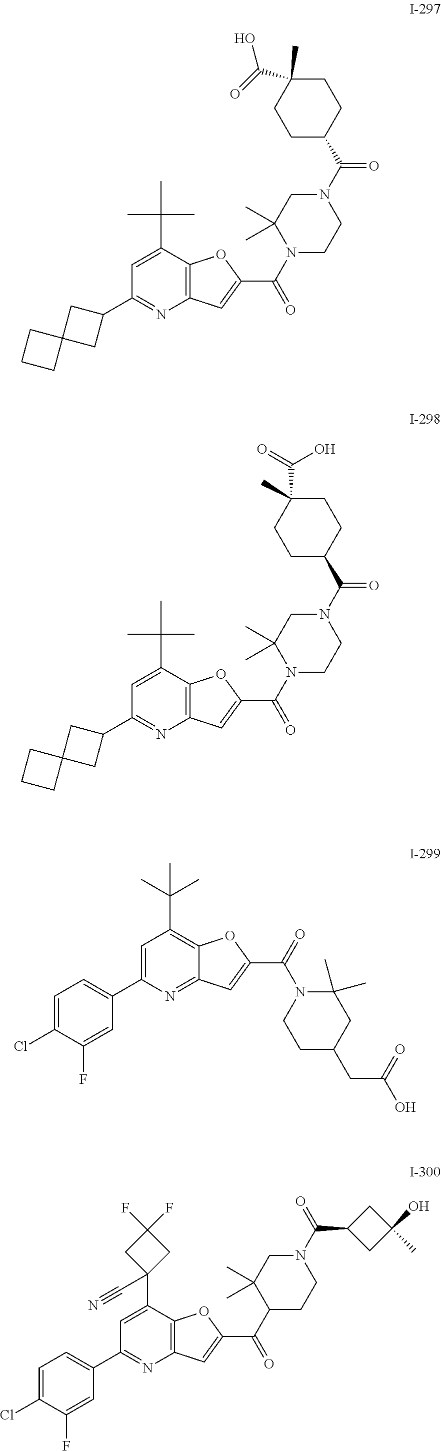 [SMILES: CC(C)(C)c1cc(C2CC3(CCC3)C2)nc2cc(C(=O)N3CCN(C(=O)[C@H]4CC[C@@](C)(C(=O)O)CC4)CC3(C)C)oc12 ; CC(C)(C)c1cc(C2CC3(CCC3)C2)nc2cc(C(=O)N3CCN(C(=O)[C@H]4CC[C@](C)(C(=O)O)CC4)CC3(C)C)oc12 ; CC(C)(C)c1cc(-c2ccc(Cl)c(F)c2)nc2cc(C(=O)N3CCC(CC(=O)O)CC3(C)C)oc12 ; CC1(C)CN(C(=O)[C@H]2C[C@@](C)(O)C2)CCC1C(=O)c1cc2nc(-c3ccc(Cl)c(F)c3)cc(C3(C#N)CC(F)(F)C3)c2o1]

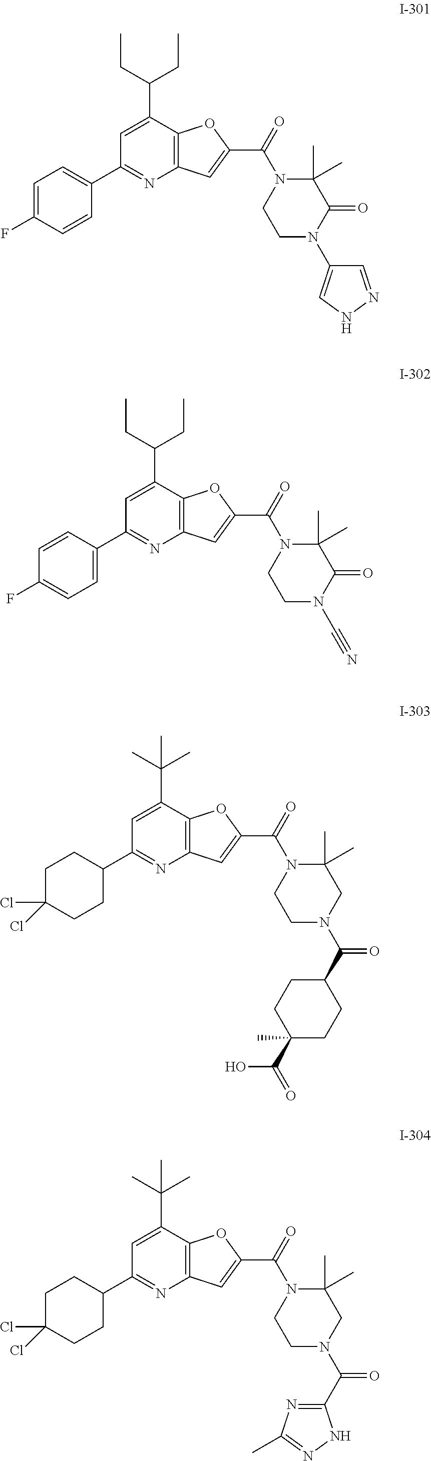 [SMILES: CCC(CC)c1cc(-c2ccc(F)cc2)nc2cc(C(=O)N3CCN(c4cn[nH]c4)C(=O)C3(C)C)oc12 ; CCC(CC)c1cc(-c2ccc(F)cc2)nc2cc(C(=O)N3CCN(C#N)C(=O)C3(C)C)oc12 ; CC(C)(C)c1cc(C2CCC(Cl)(Cl)CC2)nc2cc(C(=O)N3CCN(C(=O)[C@H]4CC[C@@](C)(C(=O)O)CC4)CC3(C)C)oc12 ; Cc1n[nH]c(C(=O)N2CCN(C(=O)c3cc4nc(C5CCC(Cl)(Cl)CC5)cc(C(C)(C)C)c4o3)C(C)(C)C2)n1]

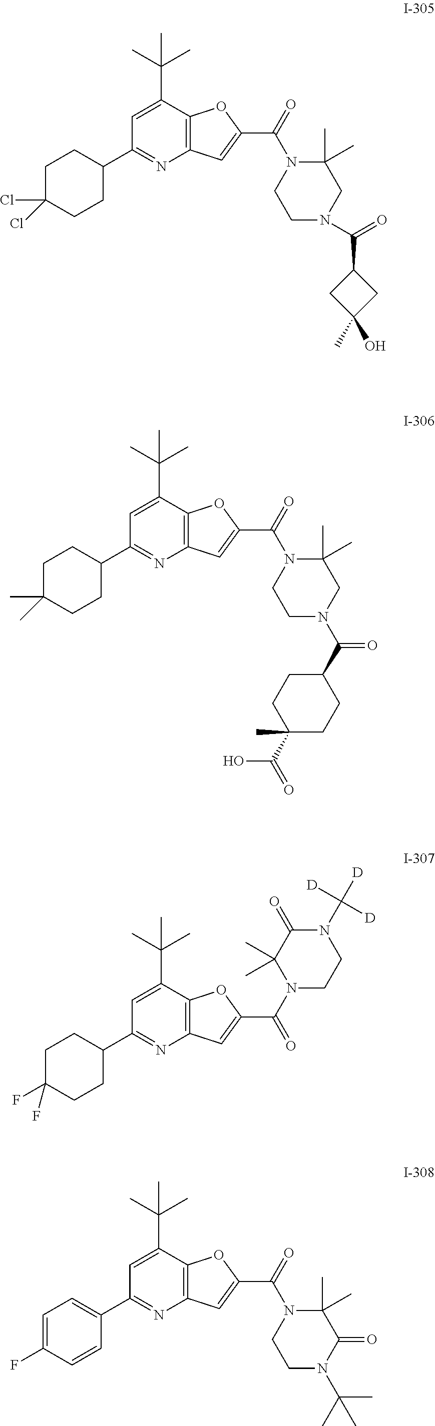 [SMILES: CC(C)(C)c1cc(C2CCC(Cl)(Cl)CC2)nc2cc(C(=O)N3CCN(C(=O)[C@H]4C[C@@](C)(O)C4)CC3(C)C)oc12 ; CC1(C)CCC(c2cc(C(C)(C)C)c3oc(C(=O)N4CCN(C(=O)[C@H]5CC[C@](C)(C(=O)O)CC5)CC4(C)C)cc3n2)CC1 ; [2H]C([2H])([2H])N1CCN(C(=O)c2cc3nc(C4CCC(F)(F)CC4)cc(C(C)(C)C)c3o2)C(C)(C)C1=O ; CC(C)(C)c1cc(-c2ccc(F)cc2)nc2cc(C(=O)N3CCN(C(C)(C)C)C(=O)C3(C)C)oc12]

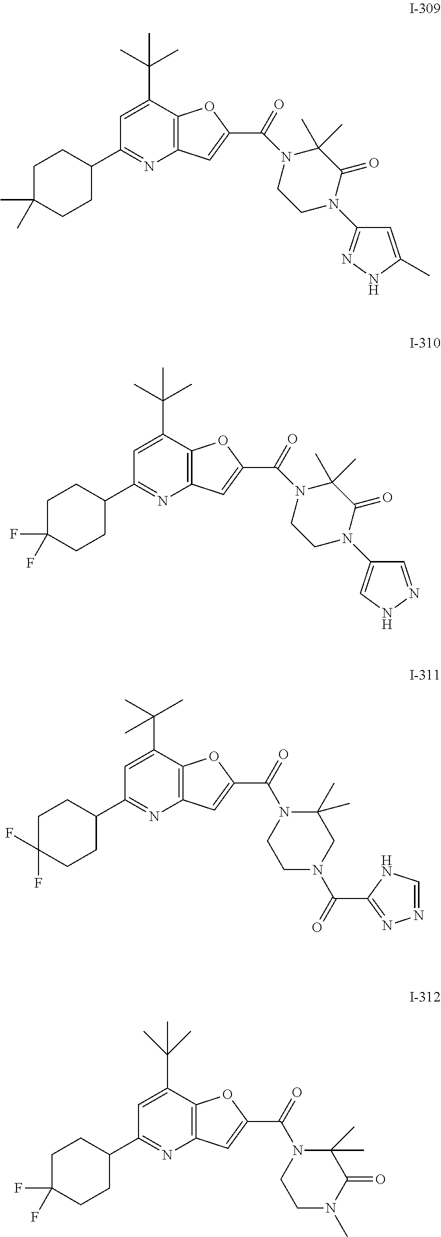

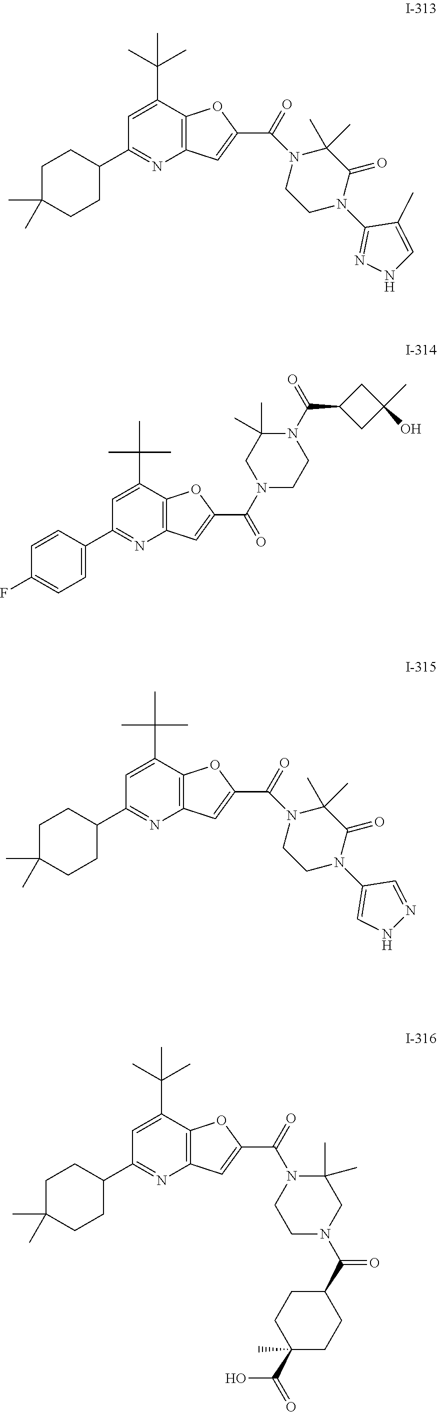

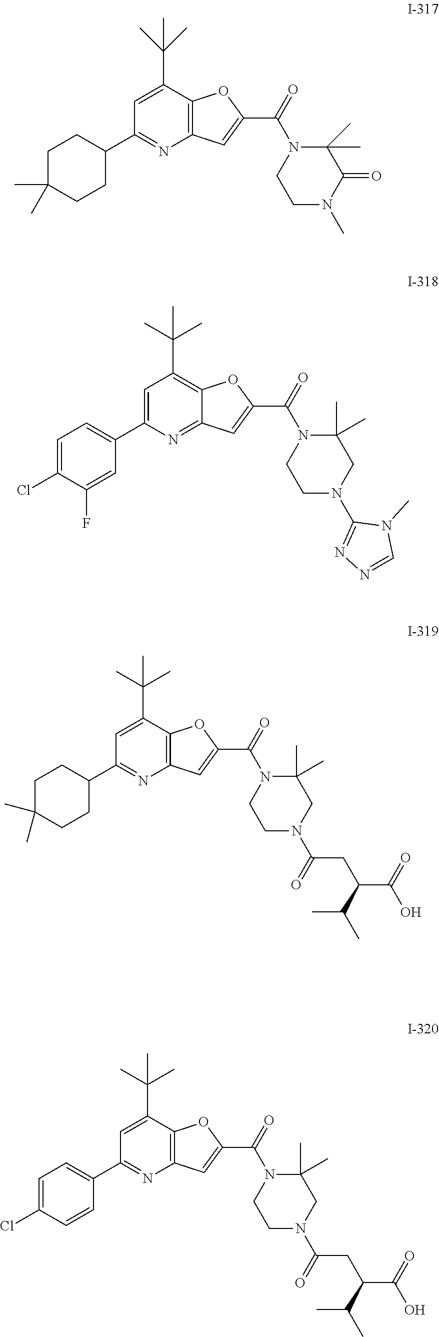

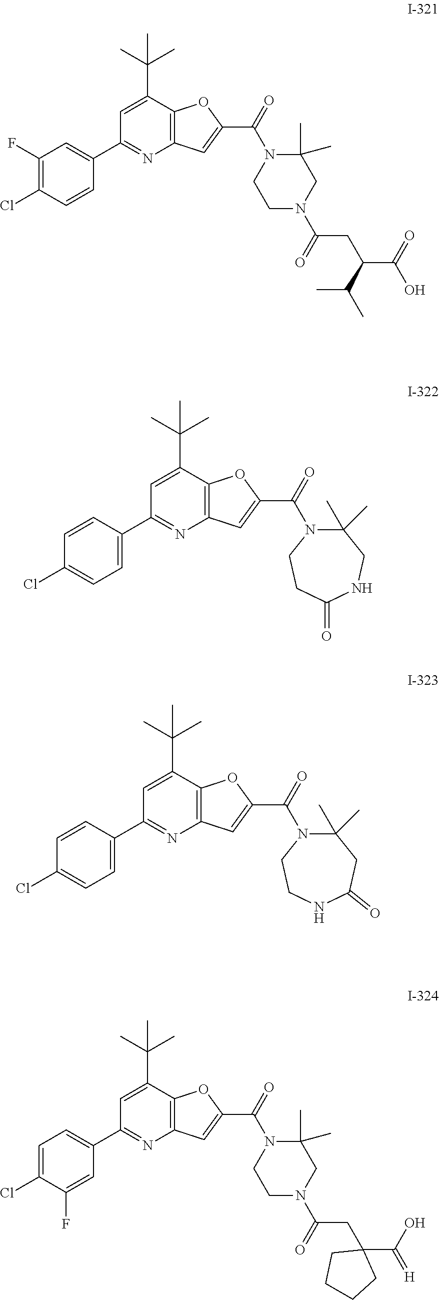

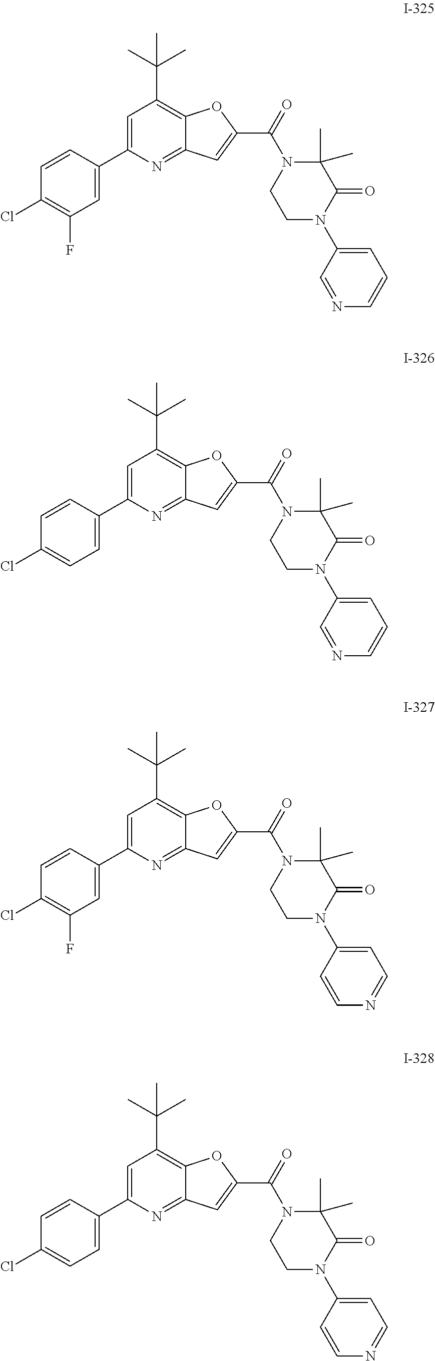 [SMILES: CC(C)(C)c1cc(-c2ccc(Cl)c(F)c2)nc2cc(C(=O)N3CCN(c4cccnc4)C(=O)C3(C)C)oc12 ; CC(C)(C)c1cc(-c2ccc(Cl)cc2)nc2cc(C(=O)N3CCN(c4cccnc4)C(=O)C3(C)C)oc12 ; CC(C)(C)c1cc(-c2ccc(Cl)c(F)c2)nc2cc(C(=O)N3CCN(c4ccncc4)C(=O)C3(C)C)oc12 ; CC(C)(C)c1cc(-c2ccc(Cl)cc2)nc2cc(C(=O)N3CCN(c4ccncc4)C(=O)C3(C)C)oc12]

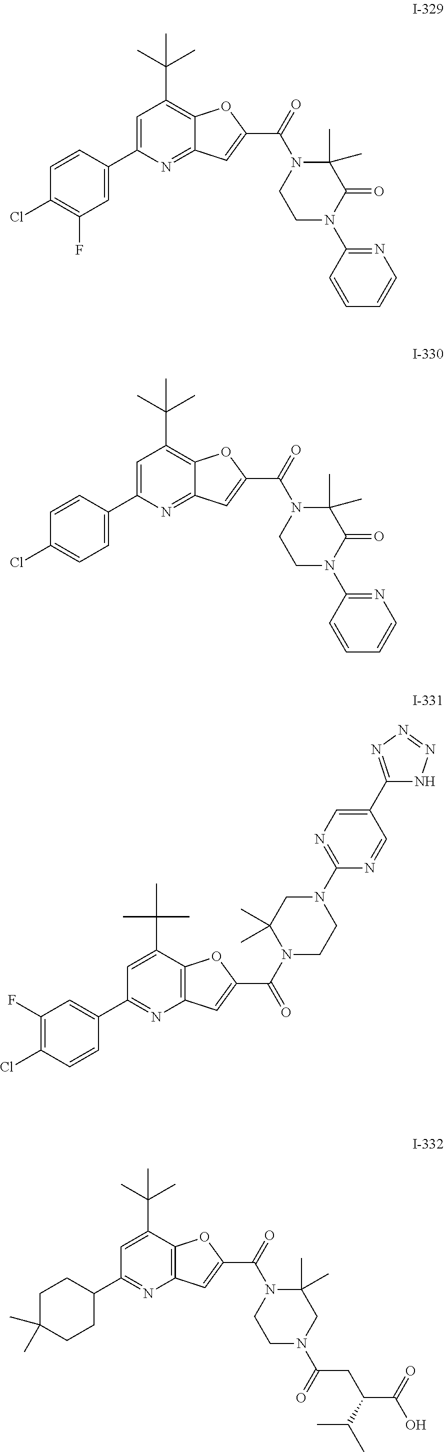 [SMILES: CC(C)(C)c1cc(-c2ccc(Cl)c(F)c2)nc2cc(C(=O)N3CCN(c4ccccn4)C(=O)C3(C)C)oc12 ; CC(C)(C)c1cc(-c2ccc(Cl)cc2)nc2cc(C(=O)N3CCN(c4ccccn4)C(=O)C3(C)C)oc12 ; CC(C)(C)c1cc(-c2ccc(Cl)c(F)c2)nc2cc(C(=O)N3CCN(c4ncc(-c5nnn[nH]5)cn4)CC3(C)C)oc12 ; CC(C)[C@H](CC(=O)N1CCN(C(=O)c2cc3nc(C4CCC(C)(C)CC4)cc(C(C)(C)C)c3o2)C(C)(C)C1)C(=O)O]

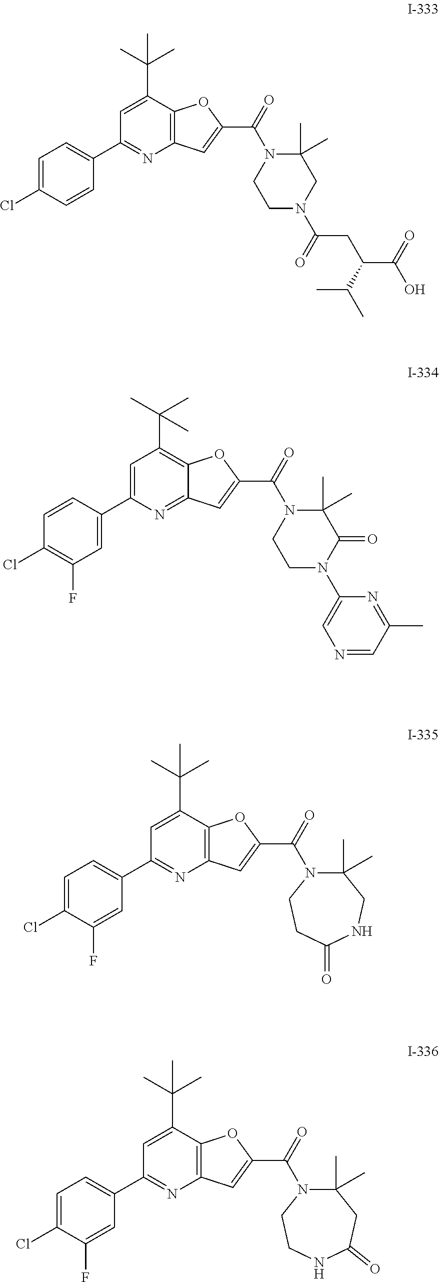 [SMILES: CC(C)[C@H](CC(=O)N1CCN(C(=O)c2cc3nc(-c4ccc(Cl)cc4)cc(C(C)(C)C)c3o2)C(C)(C)C1)C(=O)O ; Cc1cncc(N2CCN(C(=O)c3cc4nc(-c5ccc(Cl)c(F)c5)cc(C(C)(C)C)c4o3)C(C)(C)C2=O)n1 ; CC(C)(C)c1cc(-c2ccc(Cl)c(F)c2)nc2cc(C(=O)N3CCC(=O)NCC3(C)C)oc12 ; CC(C)(C)c1cc(-c2ccc(Cl)c(F)c2)nc2cc(C(=O)N3CCNC(=O)CC3(C)C)oc12]

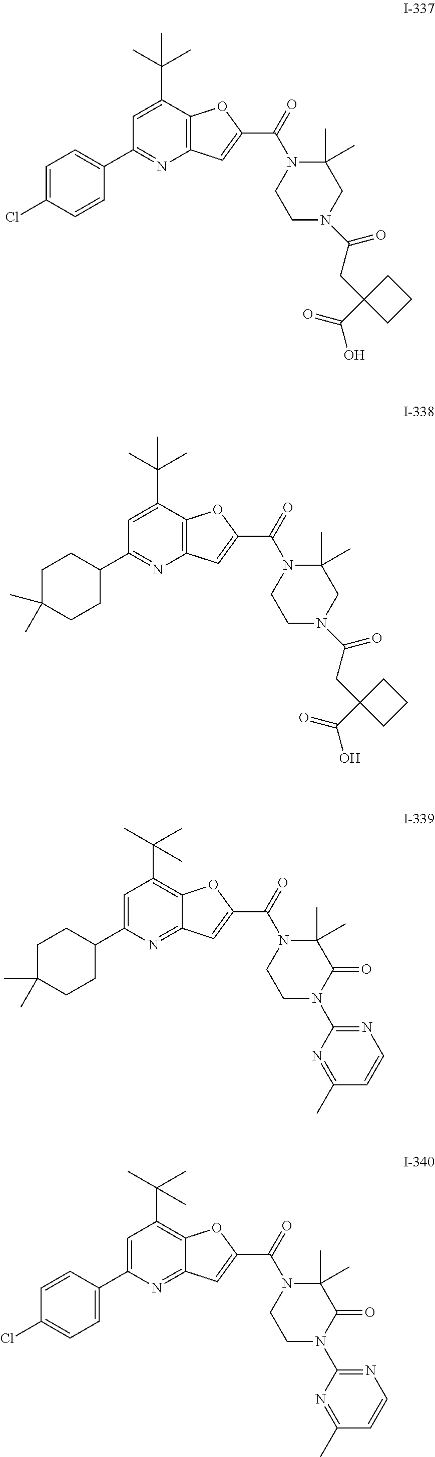 [SMILES: CC(C)(C)c1cc(-c2ccc(Cl)cc2)nc2cc(C(=O)N3CCN(C(=O)CC4(C(=O)O)CCC4)CC3(C)C)oc12 ; CC1(C)CCC(c2cc(C(C)(C)C)c3oc(C(=O)N4CCN(C(=O)CC5(C(=O)O)CCC5)CC4(C)C)cc3n2)CC1 ; Cc1ccnc(N2CCN(C(=O)c3cc4nc(C5CCC(C)(C)CC5)cc(C(C)(C)C)c4o3)C(C)(C)C2=O)n1 ; Cc1ccnc(N2CCN(C(=O)c3cc4nc(-c5ccc(Cl)cc5)cc(C(C)(C)C)c4o3)C(C)(C)C2=O)n1]

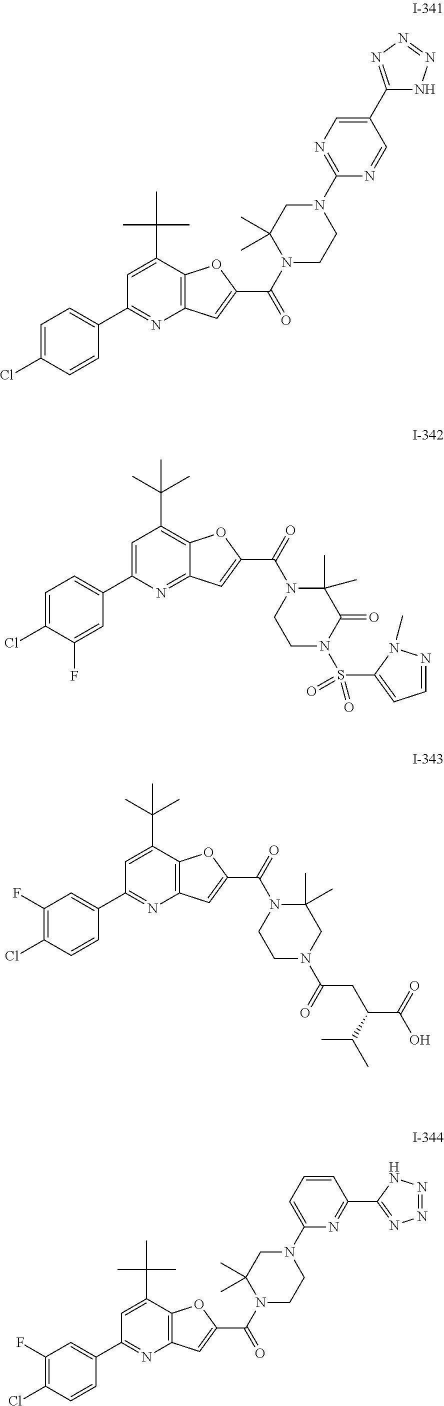 [SMILES: CC(C)(C)c1cc(-c2ccc(Cl)cc2)nc2cc(C(=O)N3CCN(c4ncc(-c5nnn[nH]5)cn4)CC3(C)C)oc12 ; Cn1nccc1S(=O)(=O)N1CCN(C(=O)c2cc3nc(-c4ccc(Cl)c(F)c4)cc(C(C)(C)C)c3o2)C(C)(C)C1=O ; CC(C)[C@H](CC(=O)N1CCN(C(=O)c2cc3nc(-c4ccc(Cl)c(F)c4)cc(C(C)(C)C)c3o2)C(C)(C)C1)C(=O)O ; CC(C)(C)c1cc(-c2ccc(Cl)c(F)c2)nc2cc(C(=O)N3CCN(c4cccc(-c5nnn[nH]5)n4)CC3(C)C)oc12]

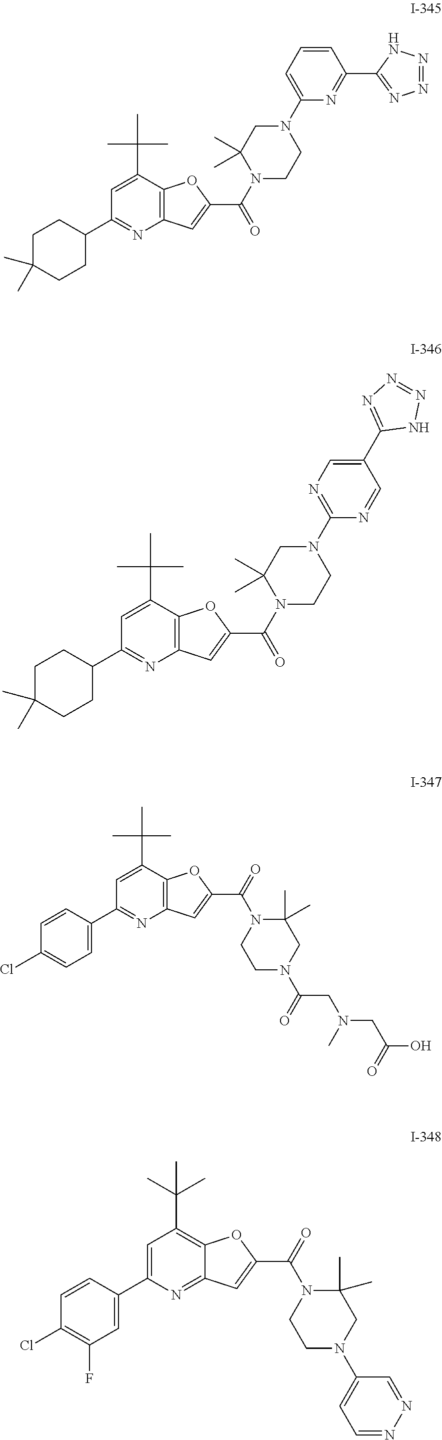 [SMILES: CC1(C)CCC(c2cc(C(C)(C)C)c3oc(C(=O)N4CCN(c5cccc(-c6nnn[nH]6)n5)CC4(C)C)cc3n2)CC1 ; CC1(C)CCC(c2cc(C(C)(C)C)c3oc(C(=O)N4CCN(c5ncc(-c6nnn[nH]6)cn5)CC4(C)C)cc3n2)CC1 ; CN(CC(=O)O)CC(=O)N1CCN(C(=O)c2cc3nc(-c4ccc(Cl)cc4)cc(C(C)(C)C)c3o2)C(C)(C)C1 ; CC(C)(C)c1cc(-c2ccc(Cl)c(F)c2)nc2cc(C(=O)N3CCN(c4ccnnc4)CC3(C)C)oc12]

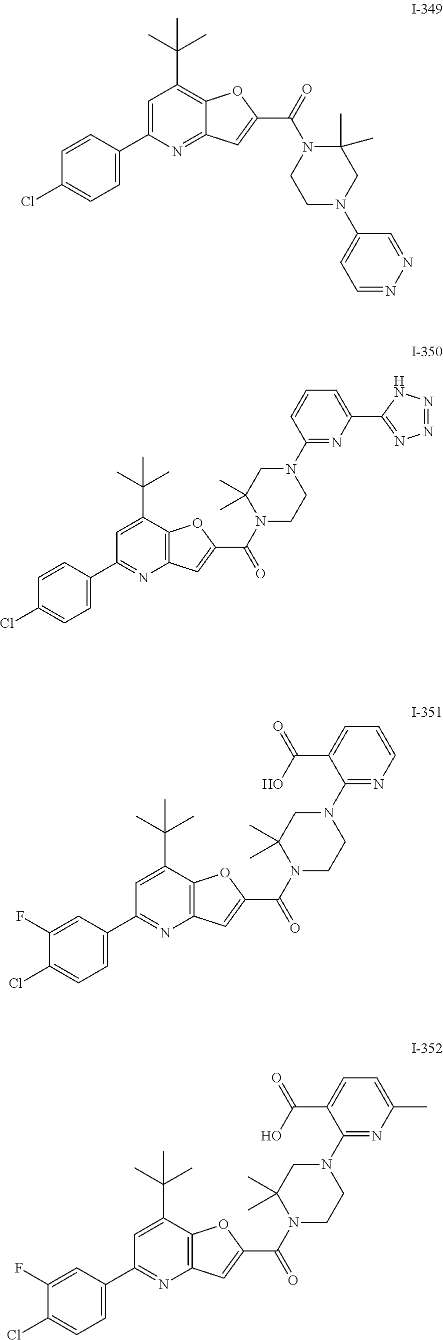 [SMILES: CC(C)(C)c1cc(-c2ccc(Cl)cc2)nc2cc(C(=O)N3CCN(c4ccnnc4)CC3(C)C)oc12 ; CC(C)(C)c1cc(-c2ccc(Cl)cc2)nc2cc(C(=O)N3CCN(c4cccc(-c5nnn[nH]5)n4)CC3(C)C)oc12 ; CC(C)(C)c1cc(-c2ccc(Cl)c(F)c2)nc2cc(C(=O)N3CCN(c4ncccc4C(=O)O)CC3(C)C)oc12 ; Cc1ccc(C(=O)O)c(N2CCN(C(=O)c3cc4nc(-c5ccc(Cl)c(F)c5)cc(C(C)(C)C)c4o3)C(C)(C)C2)n1]

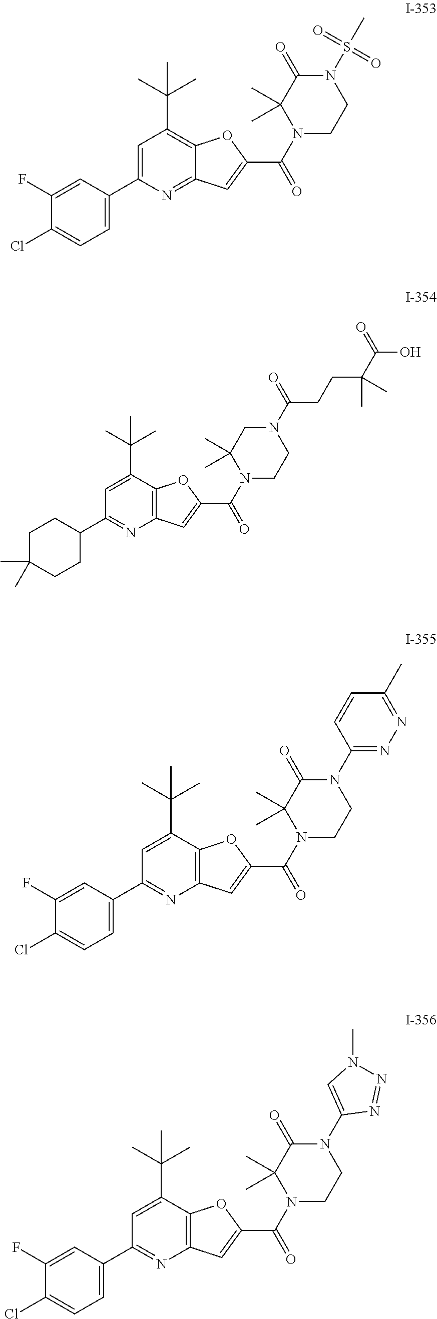 [SMILES: CC(C)(C)c1cc(-c2ccc(Cl)c(F)c2)nc2cc(C(=O)N3CCN(S(C)(=O)=O)C(=O)C3(C)C)oc12 ; CC1(C)CCC(c2cc(C(C)(C)C)c3oc(C(=O)N4CCN(C(=O)CCC(C)(C)C(=O)O)CC4(C)C)cc3n2)CC1 ; Cc1ccc(N2CCN(C(=O)c3cc4nc(-c5ccc(Cl)c(F)c5)cc(C(C)(C)C)c4o3)C(C)(C)C2=O)nn1 ; Cn1cc(N2CCN(C(=O)c3cc4nc(-c5ccc(Cl)c(F)c5)cc(C(C)(C)C)c4o3)C(C)(C)C2=O)nn1]

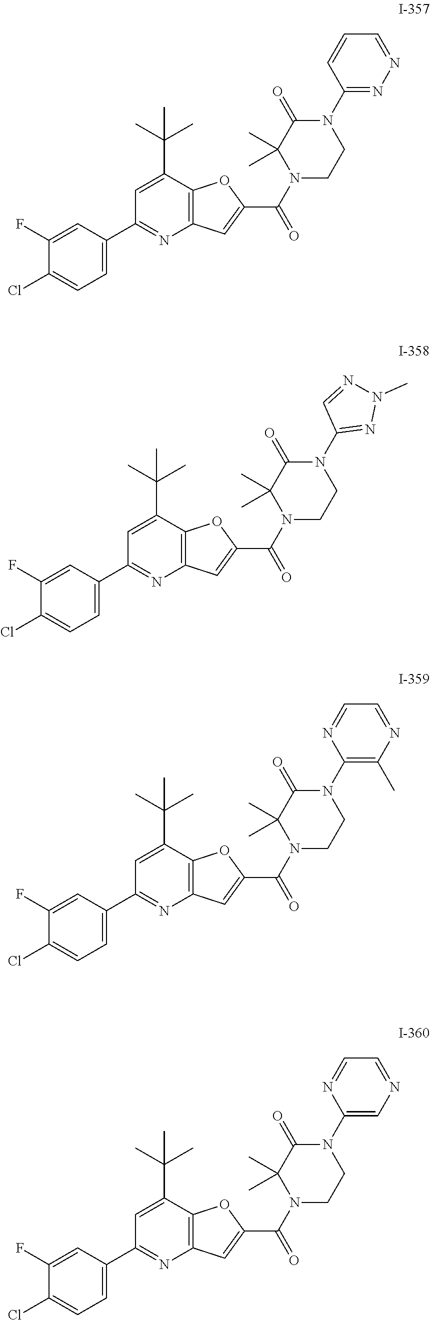 [SMILES: CC(C)(C)c1cc(-c2ccc(Cl)c(F)c2)nc2cc(C(=O)N3CCN(c4cccnn4)C(=O)C3(C)C)oc12 ; Cn1ncc(N2CCN(C(=O)c3cc4nc(-c5ccc(Cl)c(F)c5)cc(C(C)(C)C)c4o3)C(C)(C)C2=O)n1 ; Cc1nccnc1N1CCN(C(=O)c2cc3nc(-c4ccc(Cl)c(F)c4)cc(C(C)(C)C)c3o2)C(C)(C)C1=O ; CC(C)(C)c1cc(-c2ccc(Cl)c(F)c2)nc2cc(C(=O)N3CCN(c4cnccn4)C(=O)C3(C)C)oc12]

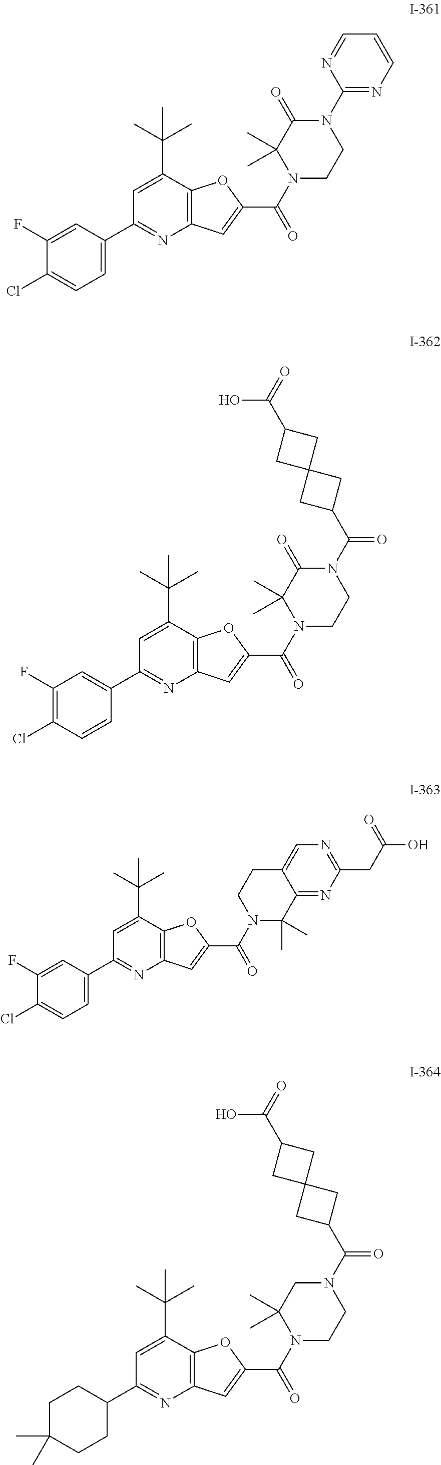 [SMILES: CC(C)(C)c1cc(-c2ccc(Cl)c(F)c2)nc2cc(C(=O)N3CCN(c4ncccn4)C(=O)C3(C)C)oc12 ; CC(C)(C)c1cc(-c2ccc(Cl)c(F)c2)nc2cc(C(=O)N3CCN(C(=O)C4CC5(CC(C(=O)O)C5)C4)C(=O)C3(C)C)oc12 ; CC(C)(C)c1cc(-c2ccc(Cl)c(F)c2)nc2cc(C(=O)N3CCc4cnc(CC(=O)O)nc4C3(C)C)oc12 ; CC1(C)CCC(c2cc(C(C)(C)C)c3oc(C(=O)N4CCN(C(=O)C5CC6(CC(C(=O)O)C6)C5)CC4(C)C)cc3n2)CC1]

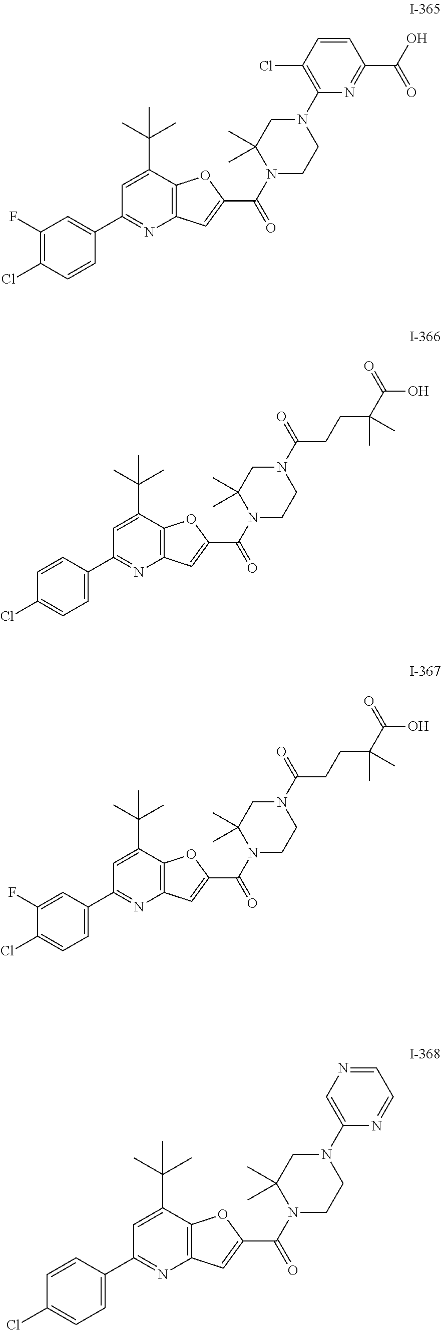 [SMILES: CC(C)(C)c1cc(-c2ccc(Cl)c(F)c2)nc2cc(C(=O)N3CCN(c4nc(C(=O)O)ccc4Cl)CC3(C)C)oc12 ; CC(C)(CCC(=O)N1CCN(C(=O)c2cc3nc(-c4ccc(Cl)cc4)cc(C(C)(C)C)c3o2)C(C)(C)C1)C(=O)O ; CC(C)(CCC(=O)N1CCN(C(=O)c2cc3nc(-c4ccc(Cl)c(F)c4)cc(C(C)(C)C)c3o2)C(C)(C)C1)C(=O)O ; CC(C)(C)c1cc(-c2ccc(Cl)cc2)nc2cc(C(=O)N3CCN(c4cnccn4)CC3(C)C)oc12]

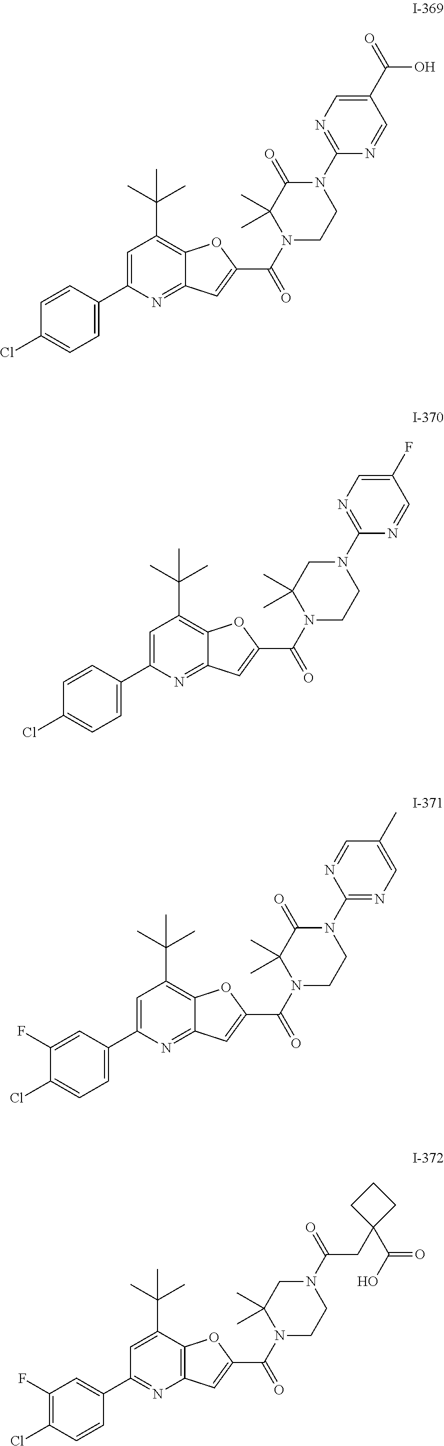 [SMILES: CC(C)(C)c1cc(-c2ccc(Cl)cc2)nc2cc(C(=O)N3CCN(c4ncc(C(=O)O)cn4)C(=O)C3(C)C)oc12 ; CC(C)(C)c1cc(-c2ccc(Cl)cc2)nc2cc(C(=O)N3CCN(c4ncc(F)cn4)CC3(C)C)oc12 ; Cc1cnc(N2CCN(C(=O)c3cc4nc(-c5ccc(Cl)c(F)c5)cc(C(C)(C)C)c4o3)C(C)(C)C2=O)nc1 ; CC(C)(C)c1cc(-c2ccc(Cl)c(F)c2)nc2cc(C(=O)N3CCN(C(=O)CC4(C(=O)O)CCC4)CC3(C)C)oc12]

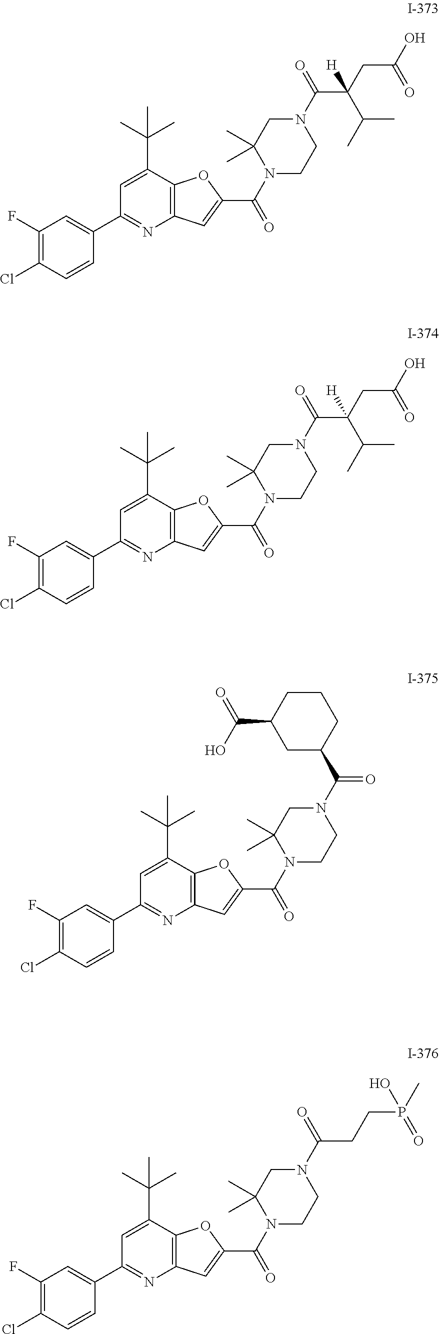 [SMILES: CC(C)[C@@H](CC(=O)O)C(=O)N1CCN(C(=O)c2cc3nc(-c4ccc(Cl)c(F)c4)cc(C(C)(C)C)c3o2)C(C)(C)C1 ; CC(C)[C@H](CC(=O)O)C(=O)N1CCN(C(=O)c2cc3nc(-c4ccc(Cl)c(F)c4)cc(C(C)(C)C)c3o2)C(C)(C)C1 ; CC(C)(C)c1cc(-c2ccc(Cl)c(F)c2)nc2cc(C(=O)N3CCN(C(=O)[C@@H]4CCC[C@H](C(=O)O)C4)CC3(C)C)oc12 ; CC(C)(C)c1cc(-c2ccc(Cl)c(F)c2)nc2cc(C(=O)N3CCN(C(=O)CCP(C)(=O)O)CC3(C)C)oc12]

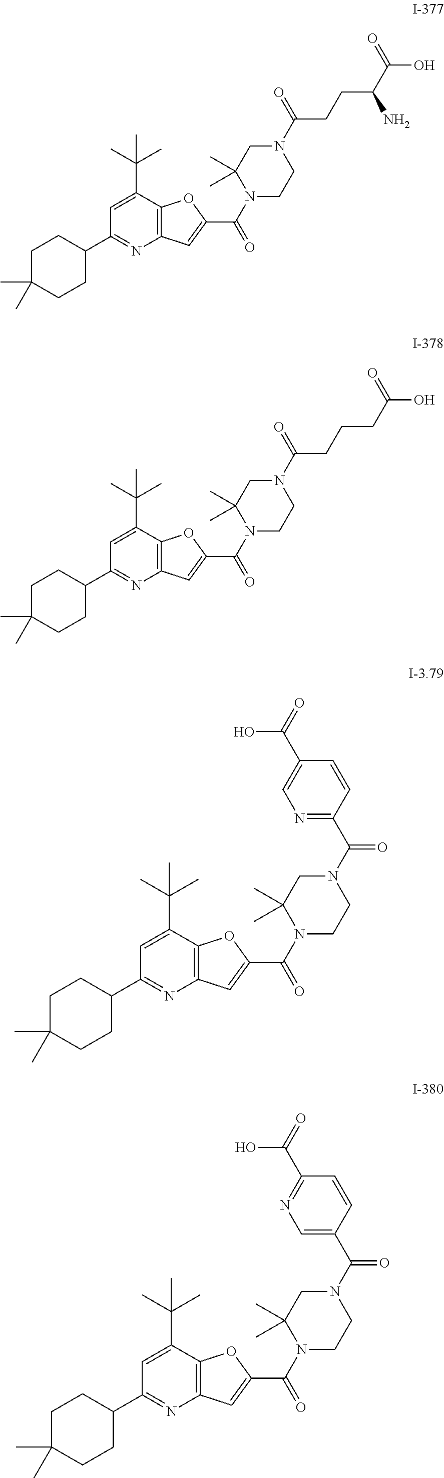 [SMILES: CC1(C)CCC(c2cc(C(C)(C)C)c3oc(C(=O)N4CCN(C(=O)CC[C@H](N)C(=O)O)CC4(C)C)cc3n2)CC1 ; CC1(C)CCC(c2cc(C(C)(C)C)c3oc(C(=O)N4CCN(C(=O)CCCC(=O)O)CC4(C)C)cc3n2)CC1 ; CC1(C)CCC(c2cc(C(C)(C)C)c3oc(C(=O)N4CCN(C(=O)c5ccc(C(=O)O)cn5)CC4(C)C)cc3n2)CC1 ; CC1(C)CCC(c2cc(C(C)(C)C)c3oc(C(=O)N4CCN(C(=O)c5ccc(C(=O)O)nc5)CC4(C)C)cc3n2)CC1]

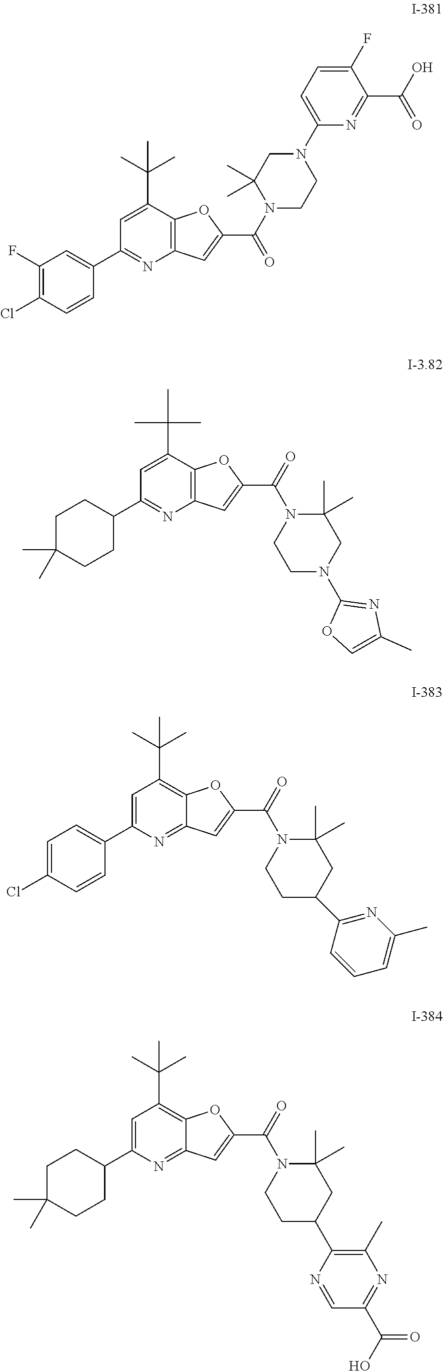

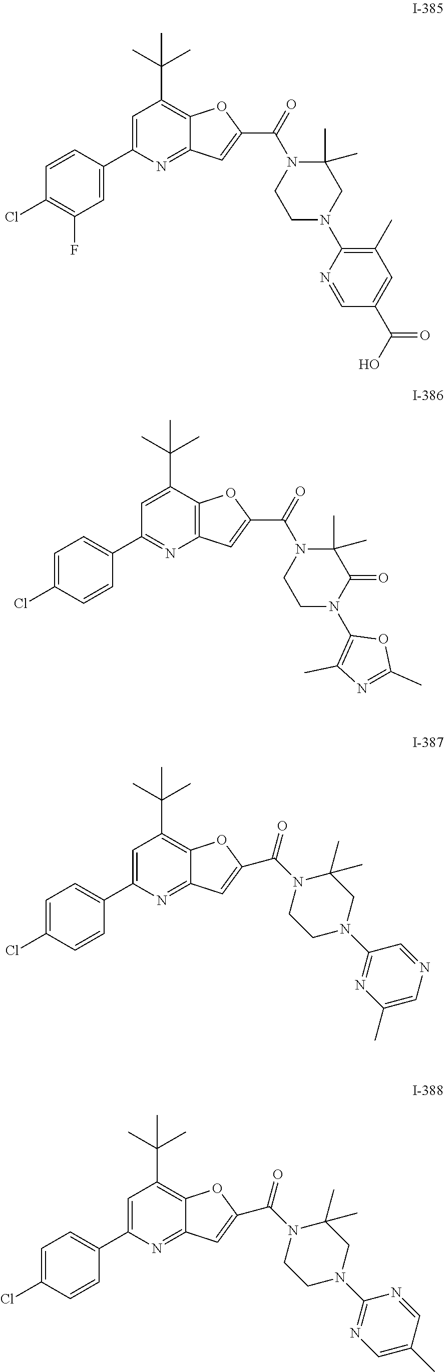 [SMILES: Cc1cc(C(=O)O)cnc1N1CCN(C(=O)c2cc3nc(-c4ccc(Cl)c(F)c4)cc(C(C)(C)C)c3o2)C(C)(C)C1 ; Cc1nc(C)c(N2CCN(C(=O)c3cc4nc(-c5ccc(Cl)cc5)cc(C(C)(C)C)c4o3)C(C)(C)C2=O)o1 ; Cc1cncc(N2CCN(C(=O)c3cc4nc(-c5ccc(Cl)cc5)cc(C(C)(C)C)c4o3)C(C)(C)C2)n1 ; Cc1cnc(N2CCN(C(=O)c3cc4nc(-c5ccc(Cl)cc5)cc(C(C)(C)C)c4o3)C(C)(C)C2)nc1]

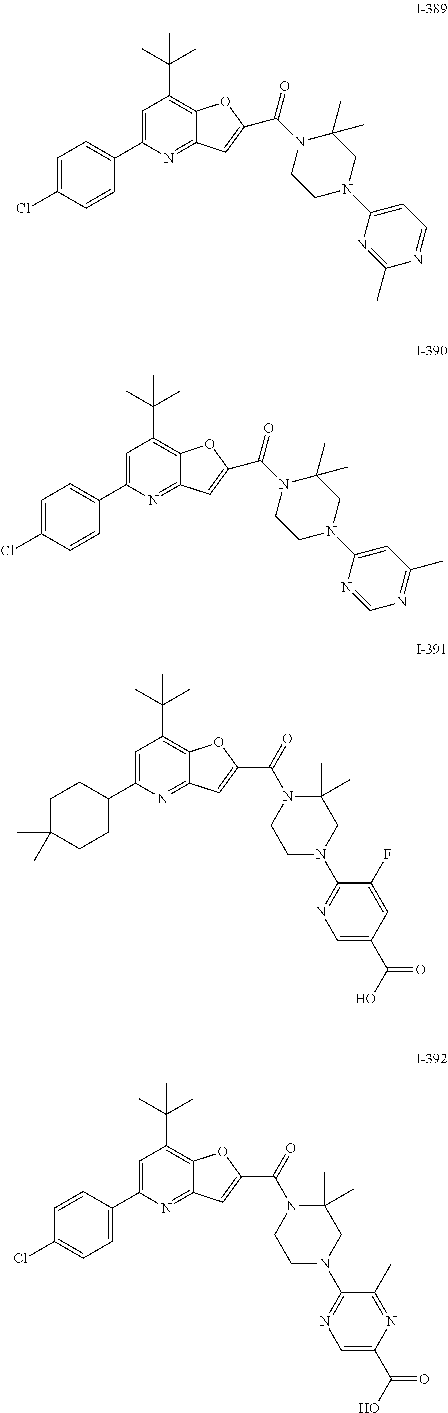

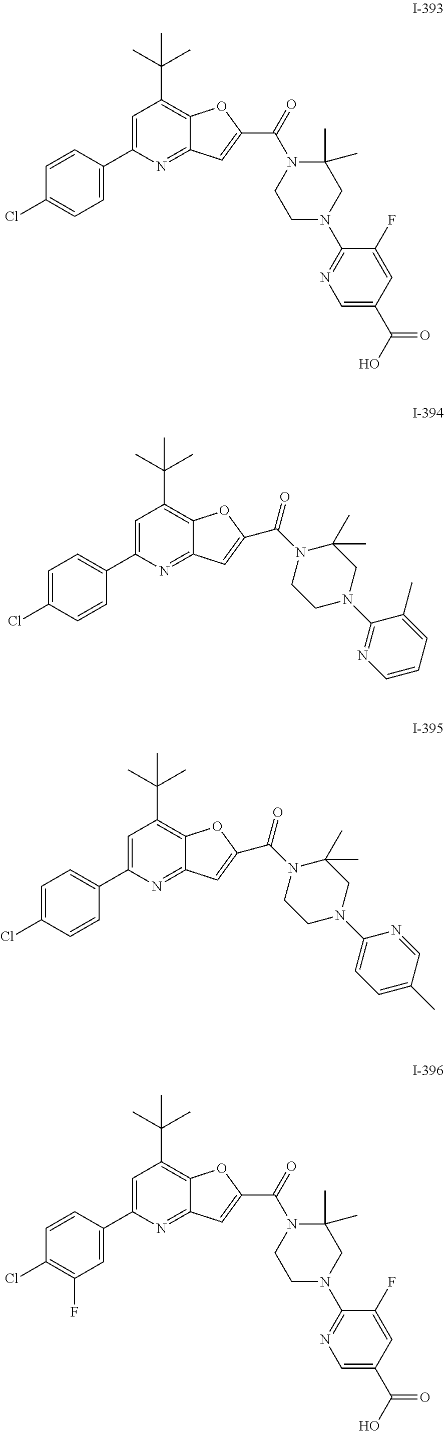 [SMILES: CC(C)(C)c1cc(-c2ccc(Cl)cc2)nc2cc(C(=O)N3CCN(c4ncc(C(=O)O)cc4F)CC3(C)C)oc12 ; Cc1cccnc1N1CCN(C(=O)c2cc3nc(-c4ccc(Cl)cc4)cc(C(C)(C)C)c3o2)C(C)(C)C1 ; Cc1ccc(N2CCN(C(=O)c3cc4nc(-c5ccc(Cl)cc5)cc(C(C)(C)C)c4o3)C(C)(C)C2)nc1 ; CC(C)(C)c1cc(-c2ccc(Cl)c(F)c2)nc2cc(C(=O)N3CCN(c4ncc(C(=O)O)cc4F)CC3(C)C)oc12]

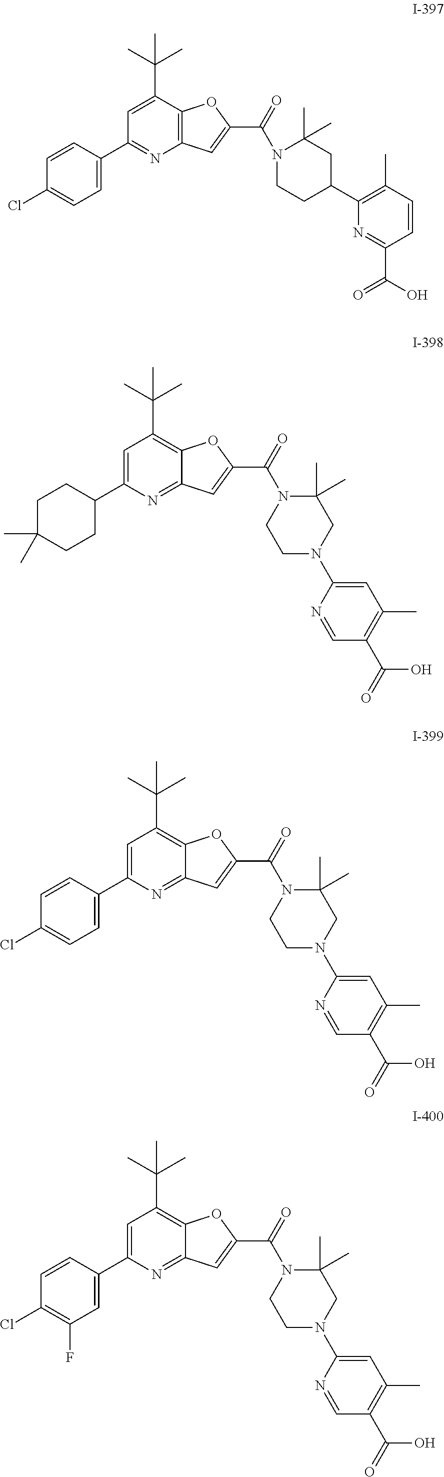 [SMILES: Cc1ccc(C(=O)O)nc1C1CCN(C(=O)c2cc3nc(-c4ccc(Cl)cc4)cc(C(C)(C)C)c3o2)C(C)(C)C1 ; Cc1cc(N2CCN(C(=O)c3cc4nc(C5CCC(C)(C)CC5)cc(C(C)(C)C)c4o3)C(C)(C)C2)ncc1C(=O)O ; Cc1cc(N2CCN(C(=O)c3cc4nc(-c5ccc(Cl)cc5)cc(C(C)(C)C)c4o3)C(C)(C)C2)ncc1C(=O)O ; Cc1cc(N2CCN(C(=O)c3cc4nc(-c5ccc(Cl)c(F)c5)cc(C(C)(C)C)c4o3)C(C)(C)C2)ncc1C(=O)O]

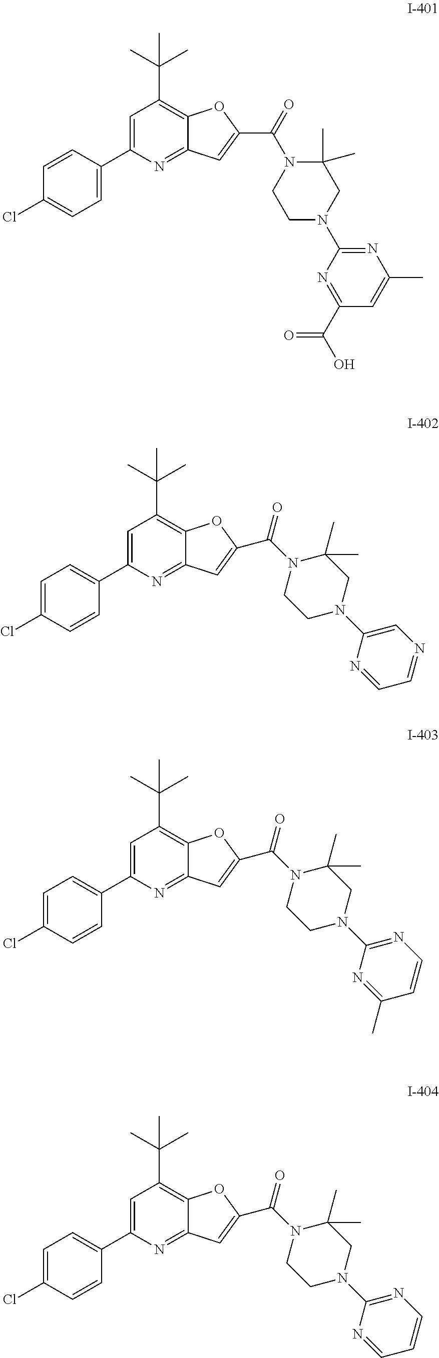 [SMILES: Cc1cc(C(=O)O)nc(N2CCN(C(=O)c3cc4nc(-c5ccc(Cl)cc5)cc(C(C)(C)C)c4o3)C(C)(C)C2)n1 ; CC(C)(C)c1cc(-c2ccc(Cl)cc2)nc2cc(C(=O)N3CCN(c4cnccn4)CC3(C)C)oc12 ; Cc1ccnc(N2CCN(C(=O)c3cc4nc(-c5ccc(Cl)cc5)cc(C(C)(C)C)c4o3)C(C)(C)C2)n1 ; CC(C)(C)c1cc(-c2ccc(Cl)cc2)nc2cc(C(=O)N3CCN(c4ncccn4)CC3(C)C)oc12]

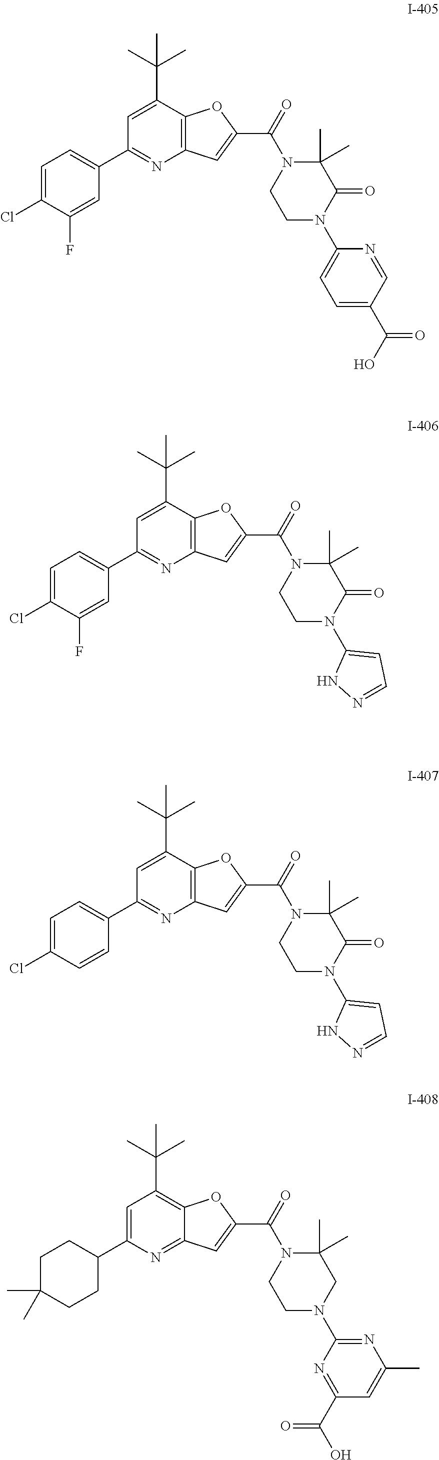

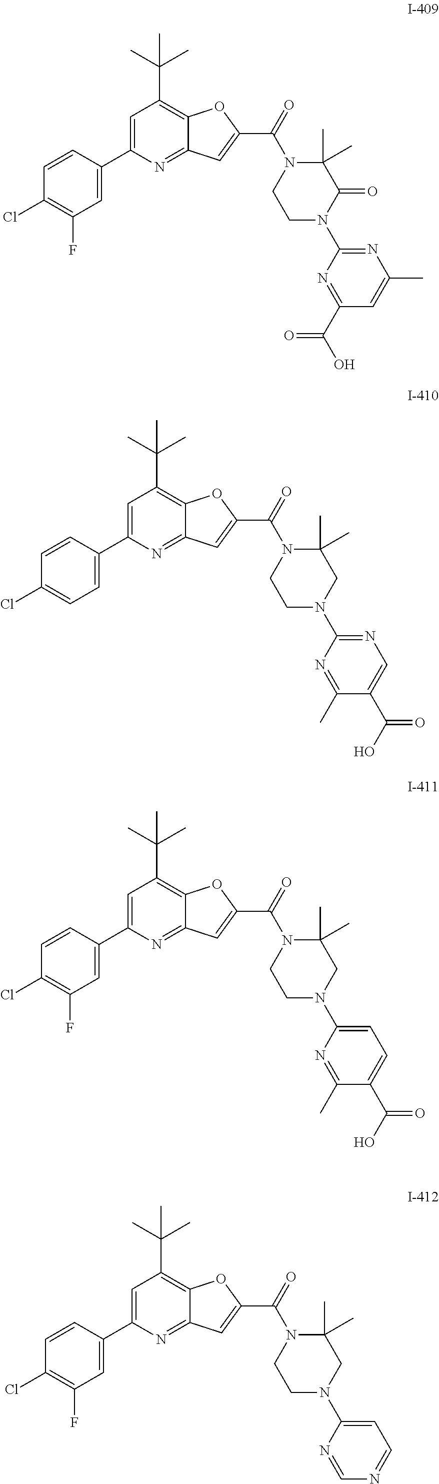 [SMILES: Cc1cc(C(=O)O)nc(N2CCN(C(=O)c3cc4nc(-c5ccc(Cl)c(F)c5)cc(C(C)(C)C)c4o3)C(C)(C)C2=O)n1 ; Cc1nc(N2CCN(C(=O)c3cc4nc(-c5ccc(Cl)cc5)cc(C(C)(C)C)c4o3)C(C)(C)C2)ncc1C(=O)O ; Cc1nc(N2CCN(C(=O)c3cc4nc(-c5ccc(Cl)c(F)c5)cc(C(C)(C)C)c4o3)C(C)(C)C2)ccc1C(=O)O ; CC(C)(C)c1cc(-c2ccc(Cl)c(F)c2)nc2cc(C(=O)N3CCN(c4ccncn4)CC3(C)C)oc12]

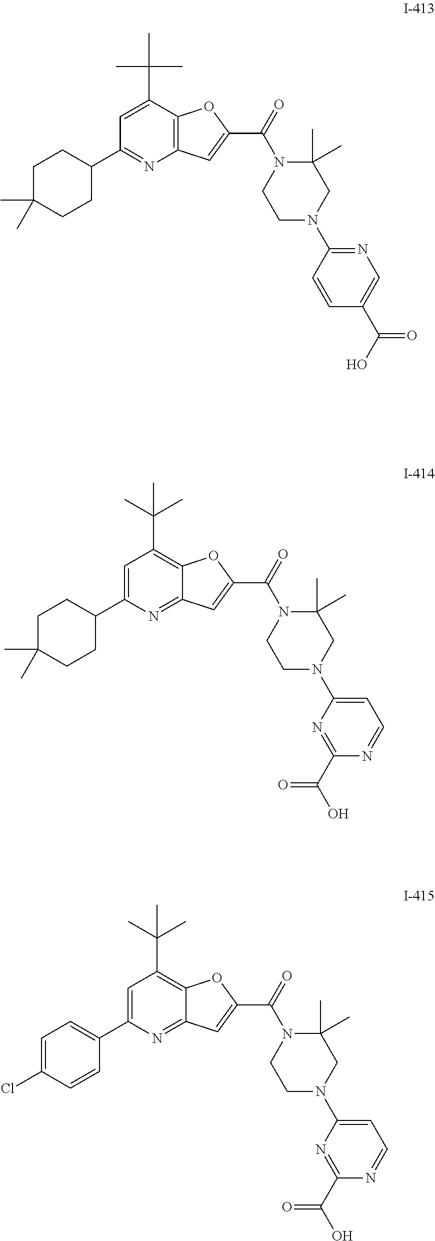 [SMILES: CC1(C)CCC(c2cc(C(C)(C)C)c3oc(C(=O)N4CCN(c5ccc(C(=O)O)cn5)CC4(C)C)cc3n2)CC1 ; CC1(C)CCC(c2cc(C(C)(C)C)c3oc(C(=O)N4CCN(c5ccnc(C(=O)O)n5)CC4(C)C)cc3n2)CC1 ; CC(C)(C)c1cc(-c2ccc(Cl)cc2)nc2cc(C(=O)N3CCN(c4ccnc(C(=O)O)n4)CC3(C)C)oc12]

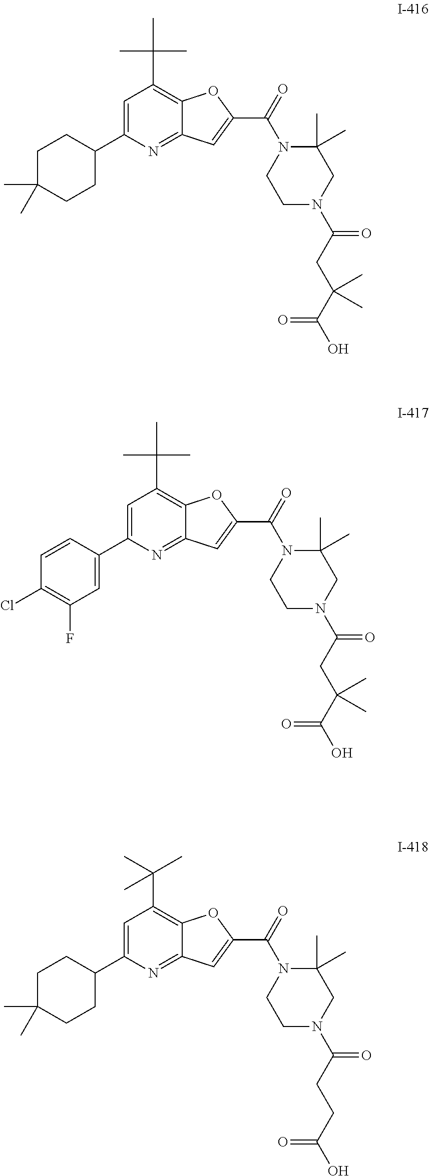 [SMILES: CC1(C)CCC(c2cc(C(C)(C)C)c3oc(C(=O)N4CCN(C(=O)CC(C)(C)C(=O)O)CC4(C)C)cc3n2)CC1 ; CC(C)(CC(=O)N1CCN(C(=O)c2cc3nc(-c4ccc(Cl)c(F)c4)cc(C(C)(C)C)c3o2)C(C)(C)C1)C(=O)O ; CC1(C)CCC(c2cc(C(C)(C)C)c3oc(C(=O)N4CCN(C(=O)CCC(=O)O)CC4(C)C)cc3n2)CC1]

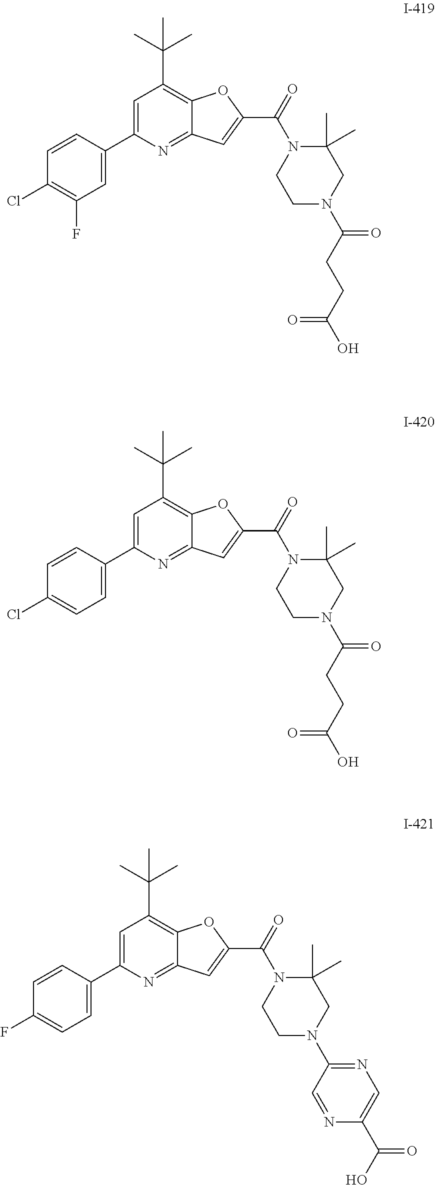 [SMILES: CC(C)(C)c1cc(-c2ccc(Cl)c(F)c2)nc2cc(C(=O)N3CCN(C(=O)CCC(=O)O)CC3(C)C)oc12 ; CC(C)(C)c1cc(-c2ccc(Cl)cc2)nc2cc(C(=O)N3CCN(C(=O)CCC(=O)O)CC3(C)C)oc12 ; CC(C)(C)c1cc(-c2ccc(F)cc2)nc2cc(C(=O)N3CCN(c4cnc(C(=O)O)cn4)CC3(C)C)oc12]

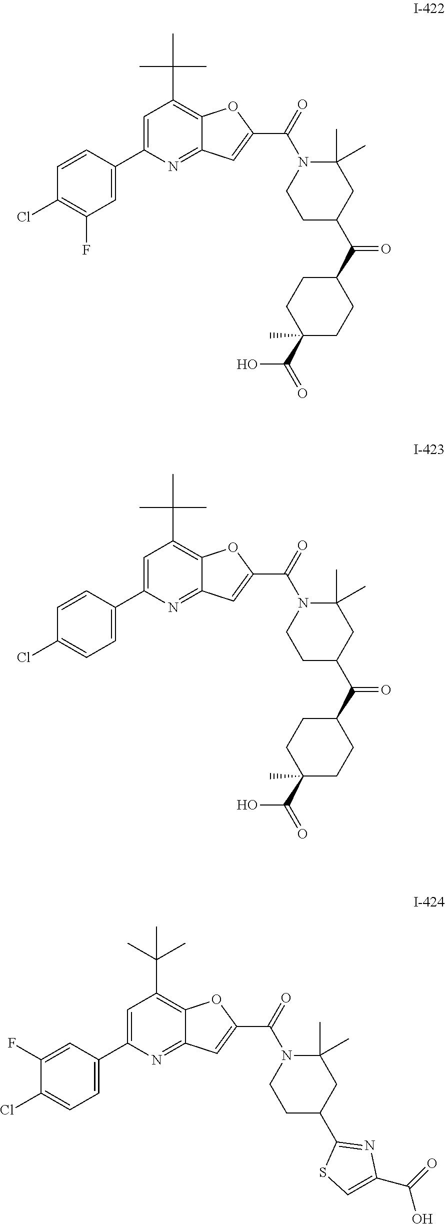 [SMILES: CC(C)(C)c1cc(-c2ccc(Cl)c(F)c2)nc2cc(C(=O)N3CCC(C(=O)[C@H]4CC[C@@](C)(C(=O)O)CC4)CC3(C)C)oc12 ; CC(C)(C)c1cc(-c2ccc(Cl)cc2)nc2cc(C(=O)N3CCC(C(=O)[C@H]4CC[C@@](C)(C(=O)O)CC4)CC3(C)C)oc12 ; CC(C)(C)c1cc(-c2ccc(Cl)c(F)c2)nc2cc(C(=O)N3CCC(c4nc(C(=O)O)cs4)CC3(C)C)oc12]

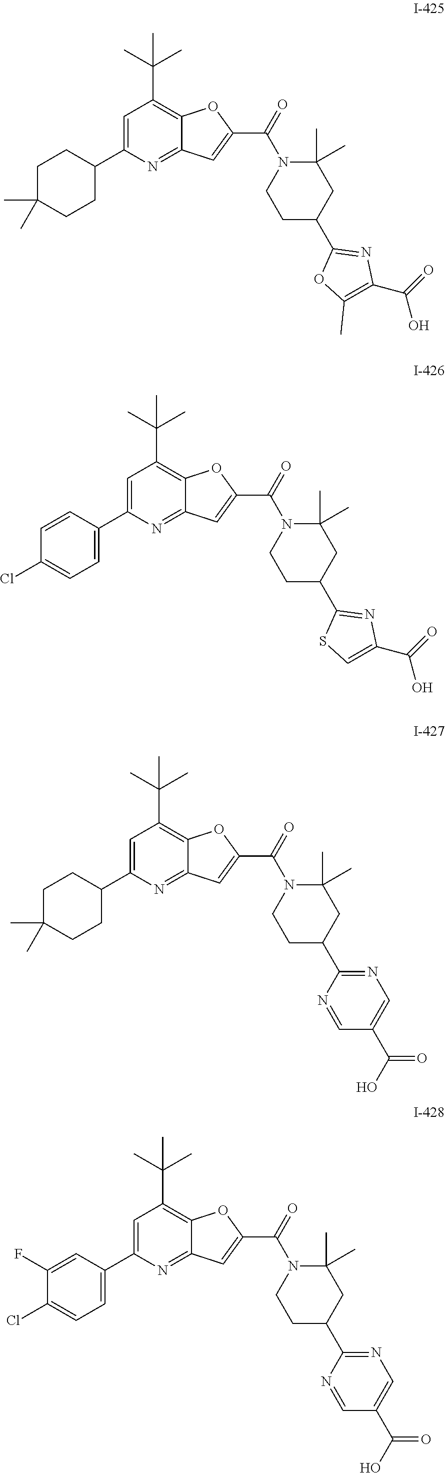 [SMILES: Cc1oc(C2CCN(C(=O)c3cc4nc(C5CCC(C)(C)CC5)cc(C(C)(C)C)c4o3)C(C)(C)C2)nc1C(=O)O ; CC(C)(C)c1cc(-c2ccc(Cl)cc2)nc2cc(C(=O)N3CCC(c4nc(C(=O)O)cs4)CC3(C)C)oc12 ; CC1(C)CCC(c2cc(C(C)(C)C)c3oc(C(=O)N4CCC(c5ncc(C(=O)O)cn5)CC4(C)C)cc3n2)CC1 ; CC(C)(C)c1cc(-c2ccc(Cl)c(F)c2)nc2cc(C(=O)N3CCC(c4ncc(C(=O)O)cn4)CC3(C)C)oc12]

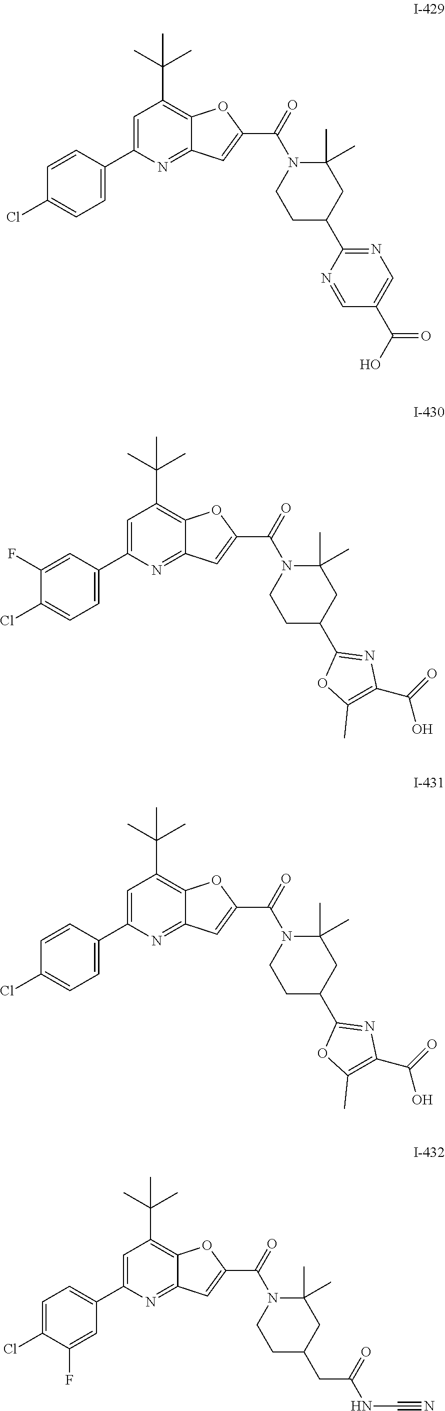 [SMILES: CC(C)(C)c1cc(-c2ccc(Cl)cc2)nc2cc(C(=O)N3CCC(c4ncc(C(=O)O)cn4)CC3(C)C)oc12 ; Cc1oc(C2CCN(C(=O)c3cc4nc(-c5ccc(Cl)c(F)c5)cc(C(C)(C)C)c4o3)C(C)(C)C2)nc1C(=O)O ; Cc1oc(C2CCN(C(=O)c3cc4nc(-c5ccc(Cl)cc5)cc(C(C)(C)C)c4o3)C(C)(C)C2)nc1C(=O)O ; CC(C)(C)c1cc(-c2ccc(Cl)c(F)c2)nc2cc(C(=O)N3CCC(CC(=O)NC#N)CC3(C)C)oc12]

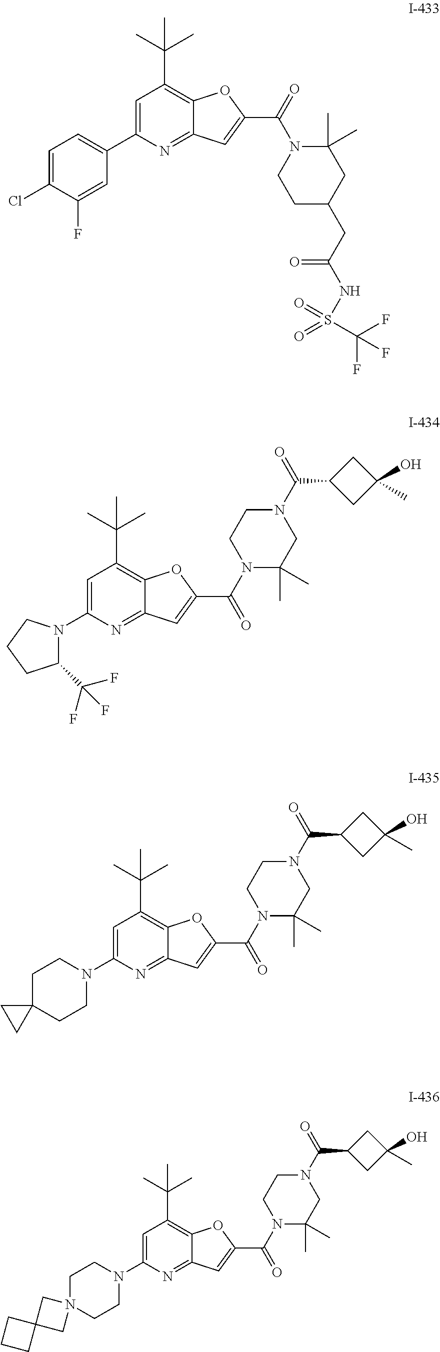

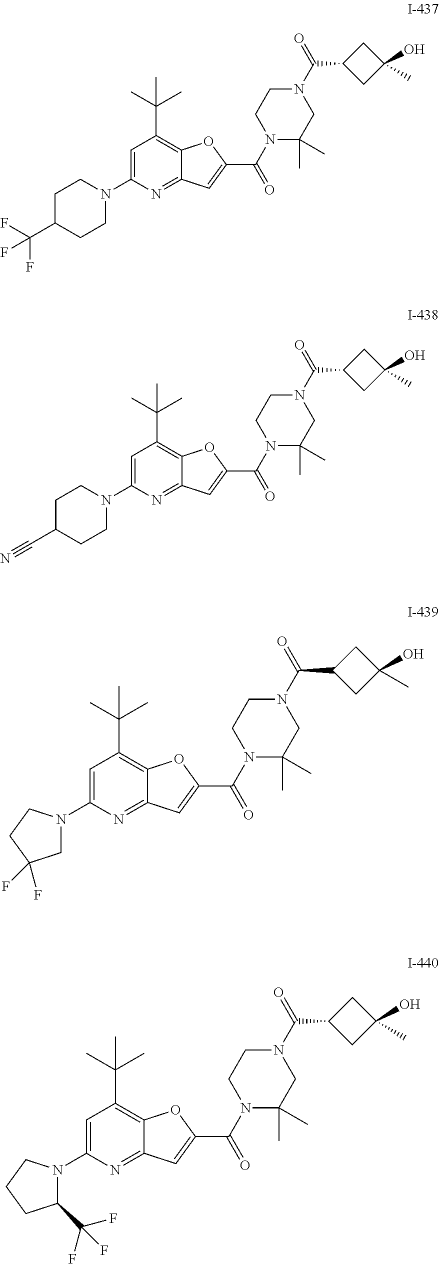

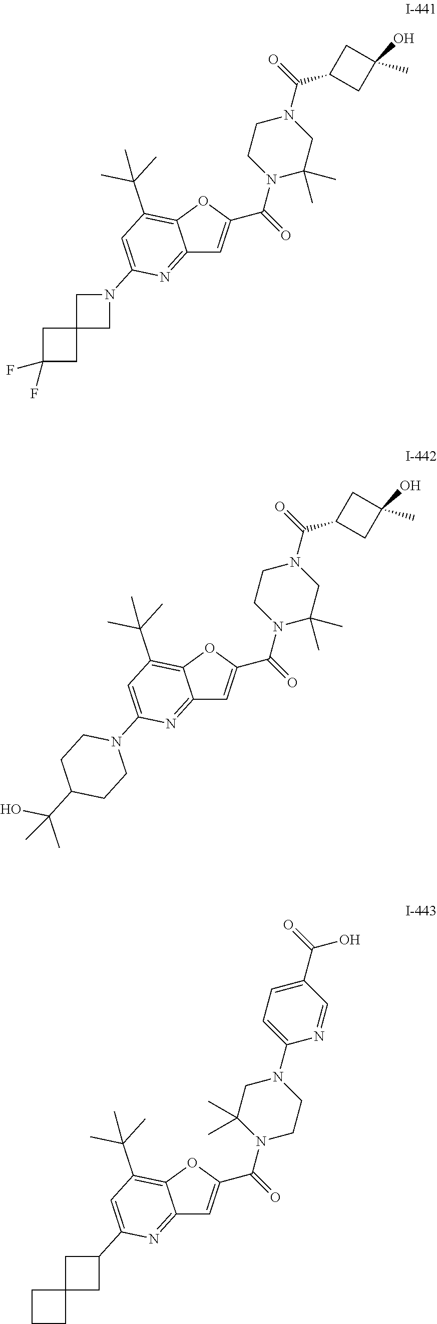 [SMILES: CC(C)(C)c1cc(N2CC3(C2)CC(F)(F)C3)nc2cc(C(=O)N3CCN(C(=O)[C@H]4C[C@](C)(O)C4)CC3(C)C)oc12 ; CC(C)(C)c1cc(N2CCC(C(C)(C)O)CC2)nc2cc(C(=O)N3CCN(C(=O)[C@H]4C[C@](C)(O)C4)CC3(C)C)oc12 ; CC(C)(C)c1cc(C2CC3(CCC3)C2)nc2cc(C(=O)N3CCN(c4ccc(C(=O)O)cn4)CC3(C)C)oc12]

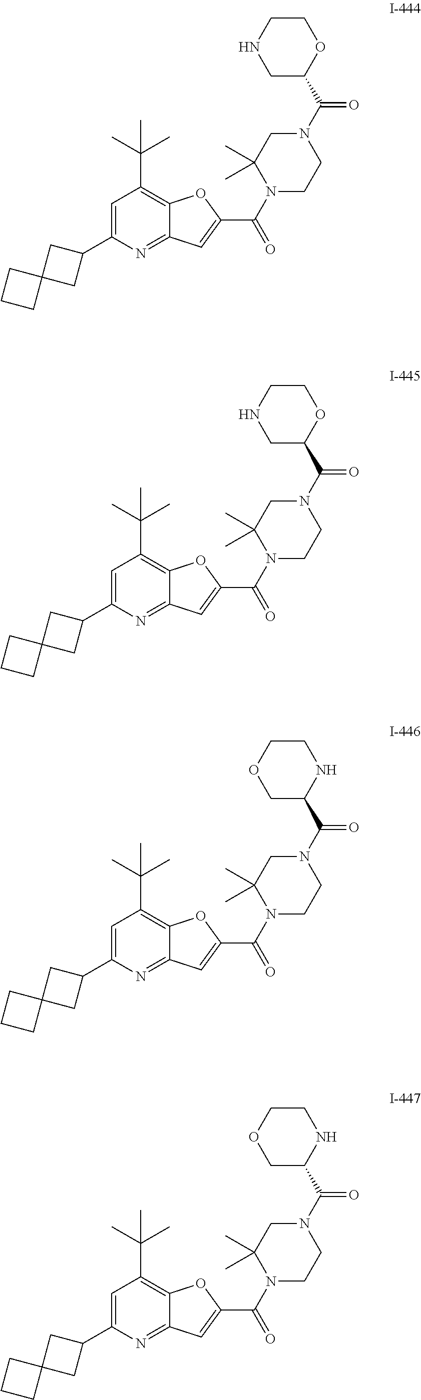 [SMILES: CC(C)(C)c1cc(C2CC3(CCC3)C2)nc2cc(C(=O)N3CCN(C(=O)[C@@H]4CNCCO4)CC3(C)C)oc12 ; CC(C)(C)c1cc(C2CC3(CCC3)C2)nc2cc(C(=O)N3CCN(C(=O)[C@H]4CNCCO4)CC3(C)C)oc12 ; CC(C)(C)c1cc(C2CC3(CCC3)C2)nc2cc(C(=O)N3CCN(C(=O)[C@H]4COCCN4)CC3(C)C)oc12 ; CC(C)(C)c1cc(C2CC3(CCC3)C2)nc2cc(C(=O)N3CCN(C(=O)[C@@H]4COCCN4)CC3(C)C)oc12]

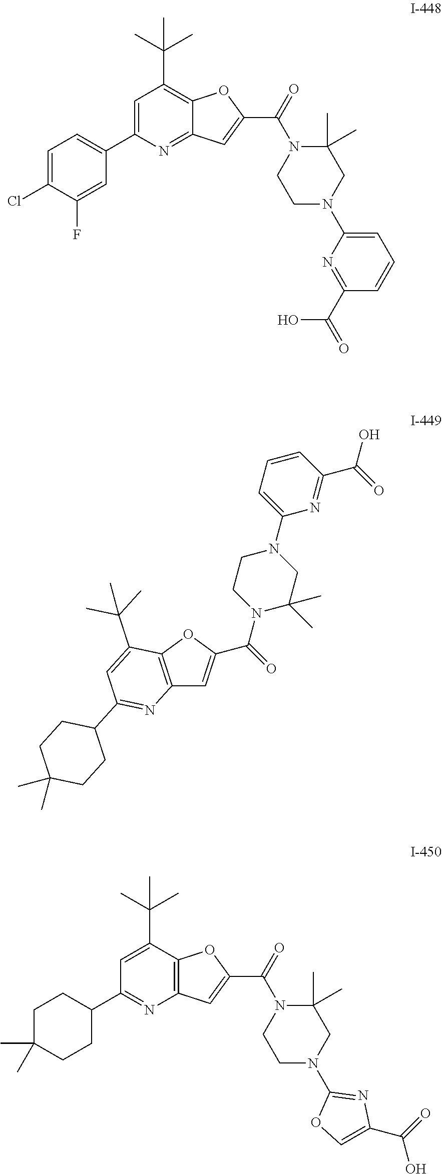 [SMILES: CC(C)(C)c1cc(-c2ccc(Cl)c(F)c2)nc2cc(C(=O)N3CCN(c4cccc(C(=O)O)n4)CC3(C)C)oc12 ; CC1(C)CCC(c2cc(C(C)(C)C)c3oc(C(=O)N4CCN(c5cccc(C(=O)O)n5)CC4(C)C)cc3n2)CC1 ; CC1(C)CCC(c2cc(C(C)(C)C)c3oc(C(=O)N4CCN(c5nc(C(=O)O)co5)CC4(C)C)cc3n2)CC1]

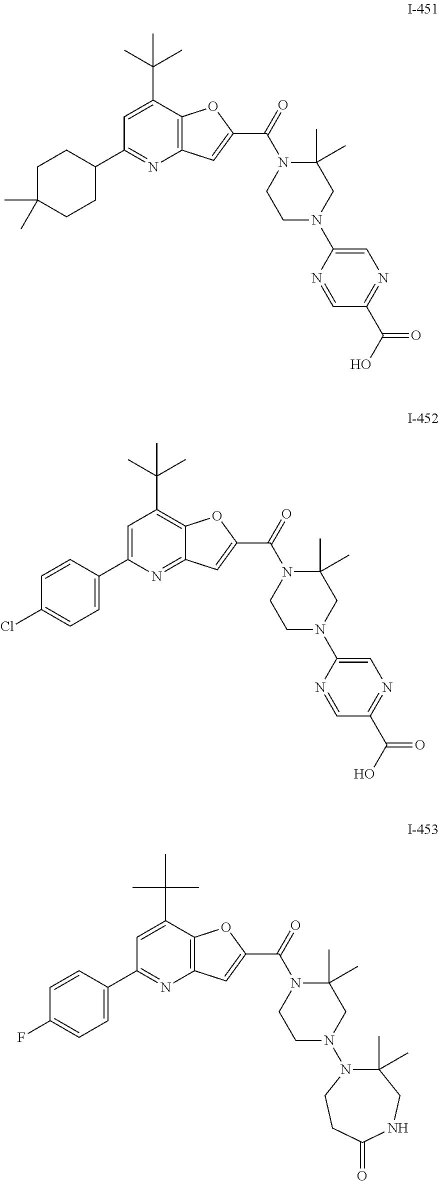 [SMILES: CC1(C)CCC(c2cc(C(C)(C)C)c3oc(C(=O)N4CCN(c5cnc(C(=O)O)cn5)CC4(C)C)cc3n2)CC1 ; CC(C)(C)c1cc(-c2ccc(Cl)cc2)nc2cc(C(=O)N3CCN(c4cnc(C(=O)O)cn4)CC3(C)C)oc12 ; CC(C)(C)c1cc(-c2ccc(F)cc2)nc2cc(C(=O)N3CCN(N4CCC(=O)NCC4(C)C)CC3(C)C)oc12]

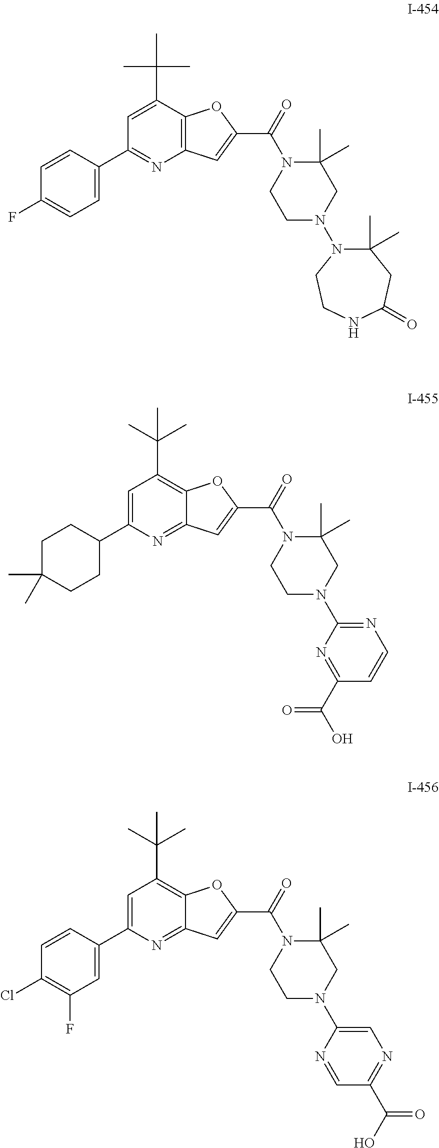 [SMILES: CC(C)(C)c1cc(-c2ccc(F)cc2)nc2cc(C(=O)N3CCN(N4CCNC(=O)CC4(C)C)CC3(C)C)oc12 ; CC1(C)CCC(c2cc(C(C)(C)C)c3oc(C(=O)N4CCN(c5nccc(C(=O)O)n5)CC4(C)C)cc3n2)CC1 ; CC(C)(C)c1cc(-c2ccc(Cl)c(F)c2)nc2cc(C(=O)N3CCN(c4cnc(C(=O)O)cn4)CC3(C)C)oc12]

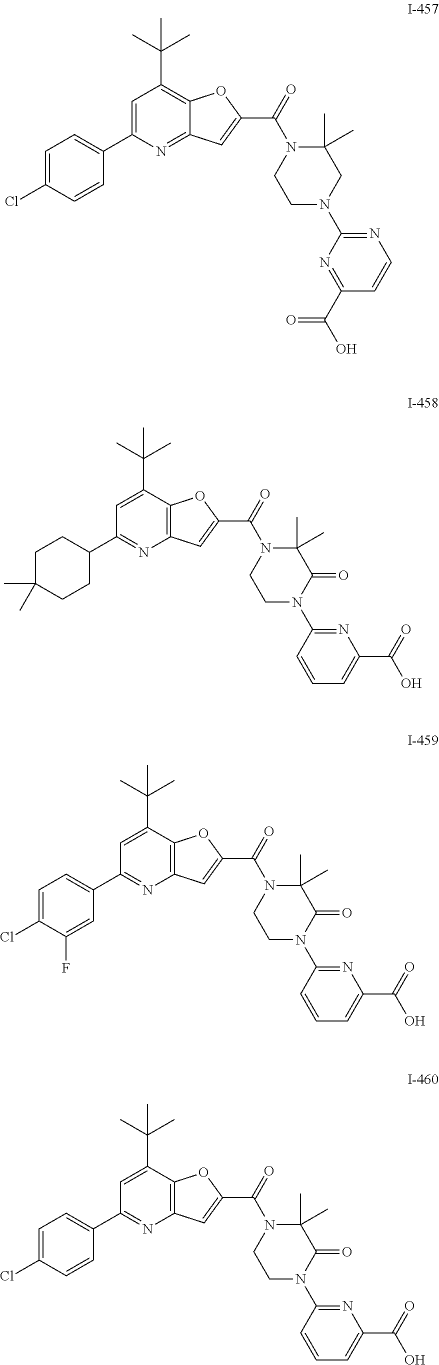 [SMILES: CC(C)(C)c1cc(-c2ccc(Cl)cc2)nc2cc(C(=O)N3CCN(c4nccc(C(=O)O)n4)CC3(C)C)oc12 ; CC1(C)CCC(c2cc(C(C)(C)C)c3oc(C(=O)N4CCN(c5cccc(C(=O)O)n5)C(=O)C4(C)C)cc3n2)CC1 ; CC(C)(C)c1cc(-c2ccc(Cl)c(F)c2)nc2cc(C(=O)N3CCN(c4cccc(C(=O)O)n4)C(=O)C3(C)C)oc12 ; CC(C)(C)c1cc(-c2ccc(Cl)cc2)nc2cc(C(=O)N3CCN(c4cccc(C(=O)O)n4)C(=O)C3(C)C)oc12]

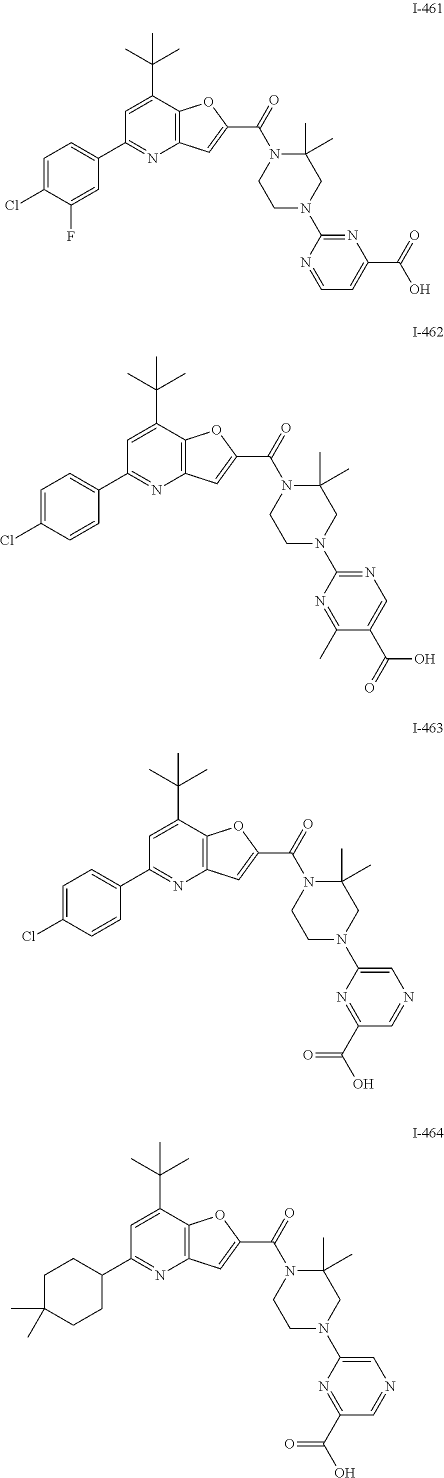 [SMILES: CC(C)(C)c1cc(-c2ccc(Cl)c(F)c2)nc2cc(C(=O)N3CCN(c4nccc(C(=O)O)n4)CC3(C)C)oc12 ; Cc1nc(N2CCN(C(=O)c3cc4nc(-c5ccc(Cl)cc5)cc(C(C)(C)C)c4o3)C(C)(C)C2)ncc1C(=O)O ; CC(C)(C)c1cc(-c2ccc(Cl)cc2)nc2cc(C(=O)N3CCN(c4cncc(C(=O)O)n4)CC3(C)C)oc12 ; CC1(C)CCC(c2cc(C(C)(C)C)c3oc(C(=O)N4CCN(c5cncc(C(=O)O)n5)CC4(C)C)cc3n2)CC1]

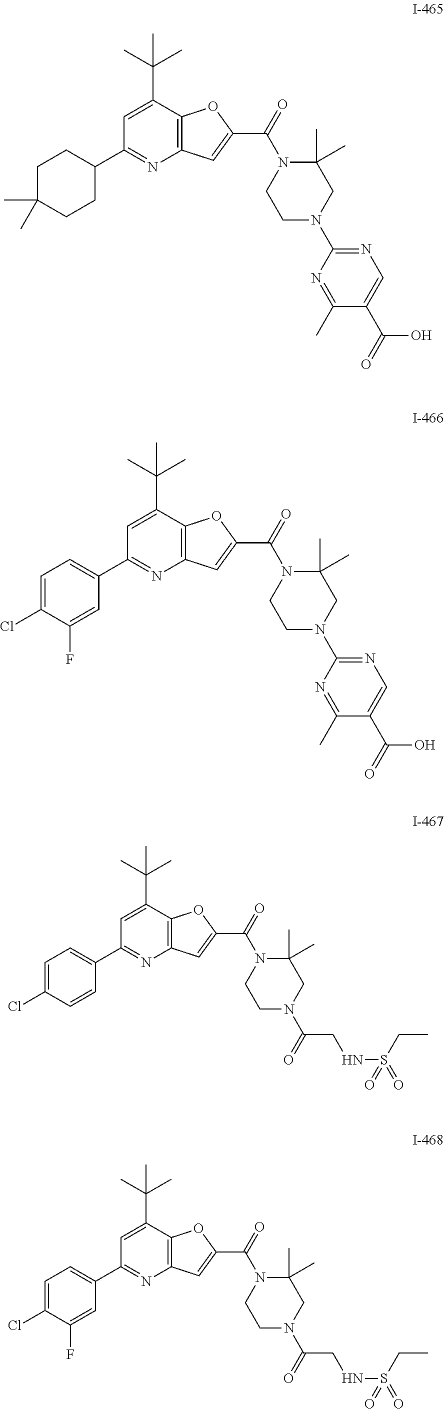 [SMILES: Cc1nc(N2CCN(C(=O)c3cc4nc(C5CCC(C)(C)CC5)cc(C(C)(C)C)c4o3)C(C)(C)C2)ncc1C(=O)O ; Cc1nc(N2CCN(C(=O)c3cc4nc(-c5ccc(Cl)c(F)c5)cc(C(C)(C)C)c4o3)C(C)(C)C2)ncc1C(=O)O ; CCS(=O)(=O)NCC(=O)N1CCN(C(=O)c2cc3nc(-c4ccc(Cl)cc4)cc(C(C)(C)C)c3o2)C(C)(C)C1 ; CCS(=O)(=O)NCC(=O)N1CCN(C(=O)c2cc3nc(-c4ccc(Cl)c(F)c4)cc(C(C)(C)C)c3o2)C(C)(C)C1]

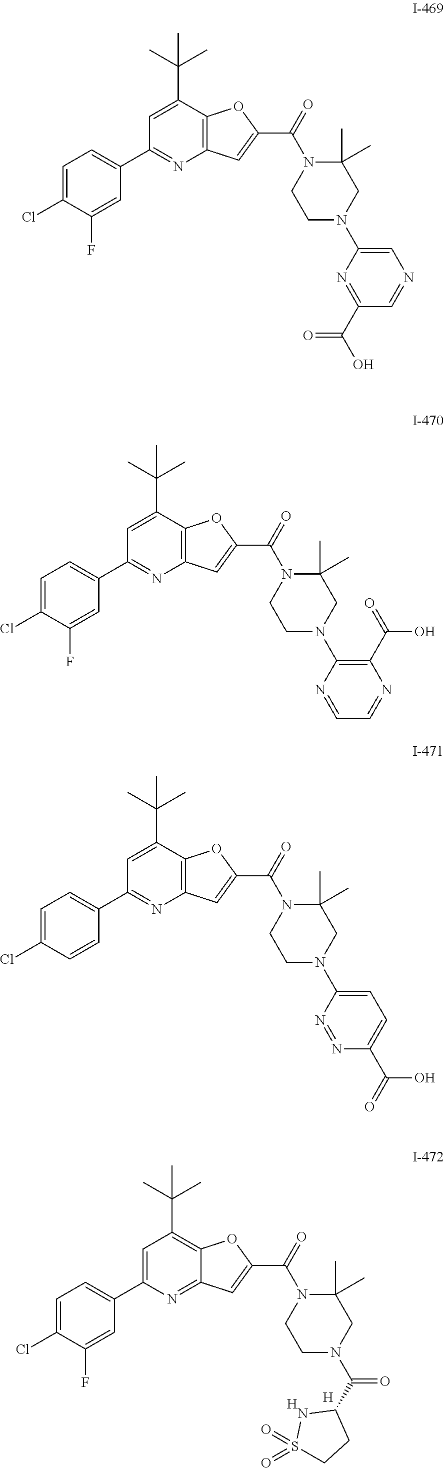

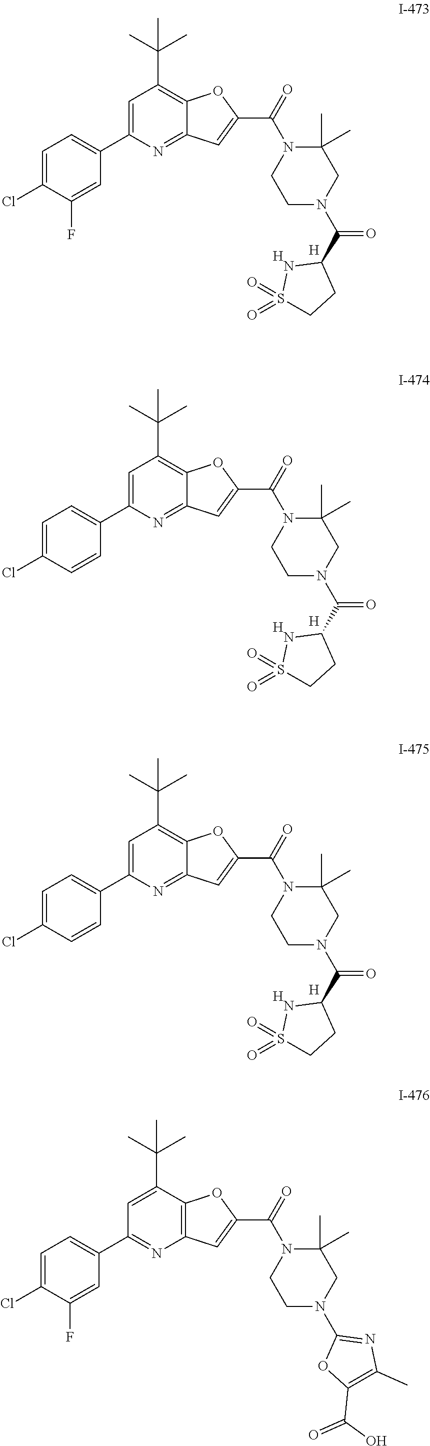

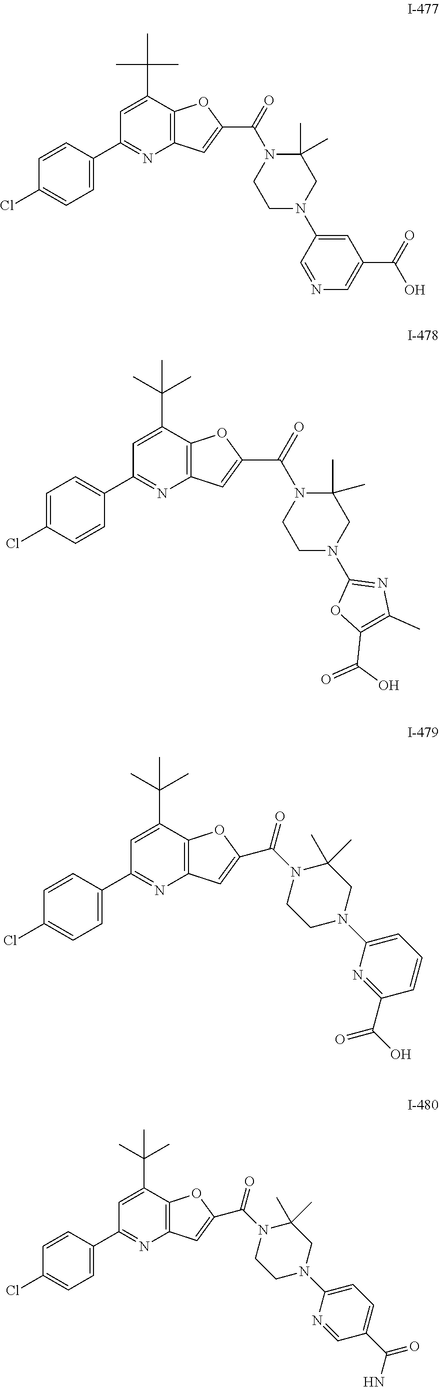 [SMILES: CC(C)(C)c1cc(-c2ccc(Cl)cc2)nc2cc(C(=O)N3CCN(c4cncc(C(=O)O)c4)CC3(C)C)oc12 ; Cc1nc(N2CCN(C(=O)c3cc4nc(-c5ccc(Cl)cc5)cc(C(C)(C)C)c4o3)C(C)(C)C2)oc1C(=O)O ; CC(C)(C)c1cc(-c2ccc(Cl)cc2)nc2cc(C(=O)N3CCN(c4cccc(C(=O)O)n4)CC3(C)C)oc12 ; CC(C)(C)c1cc(-c2ccc(Cl)cc2)nc2cc(C(=O)N3CCN(c4ccc(C(N)=O)cn4)CC3(C)C)oc12]

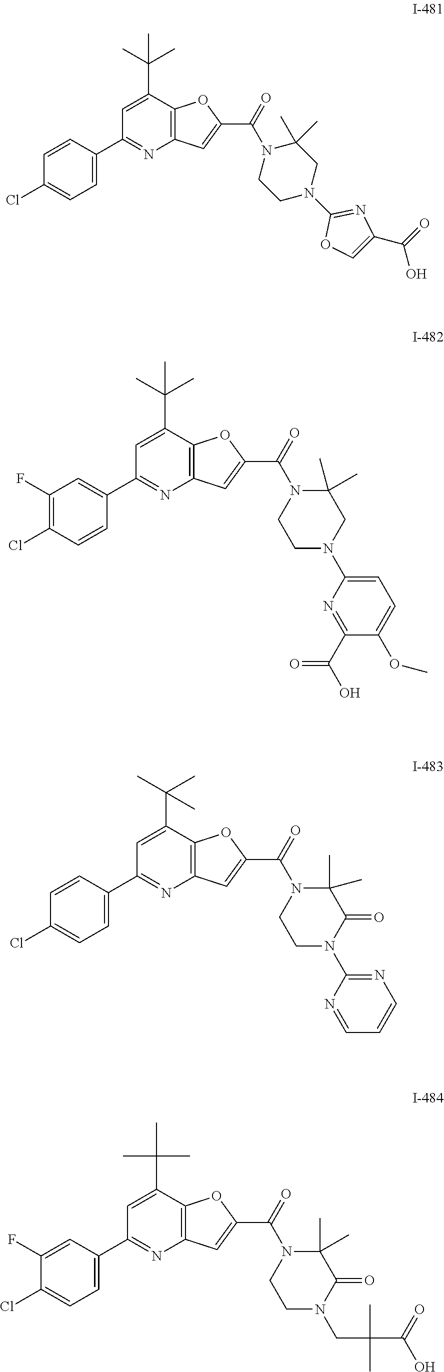 [SMILES: CC(C)(C)c1cc(-c2ccc(Cl)cc2)nc2cc(C(=O)N3CCN(c4nc(C(=O)O)co4)CC3(C)C)oc12 ; COc1ccc(N2CCN(C(=O)c3cc4nc(-c5ccc(Cl)c(F)c5)cc(C(C)(C)C)c4o3)C(C)(C)C2)nc1C(=O)O ; CC(C)(C)c1cc(-c2ccc(Cl)cc2)nc2cc(C(=O)N3CCN(c4ncccn4)C(=O)C3(C)C)oc12 ; CC(C)(CN1CCN(C(=O)c2cc3nc(-c4ccc(Cl)c(F)c4)cc(C(C)(C)C)c3o2)C(C)(C)C1=O)C(=O)O]

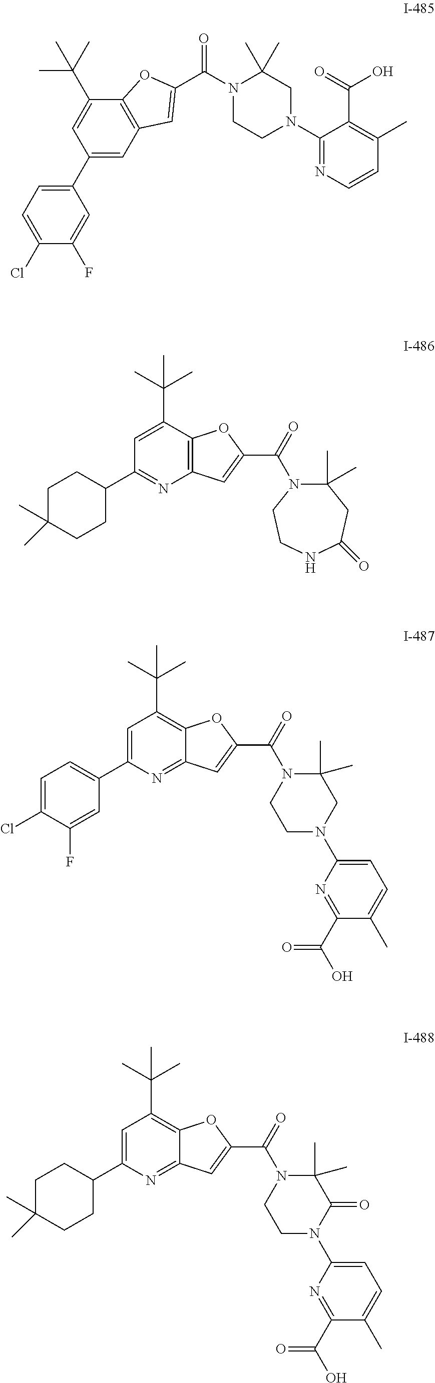

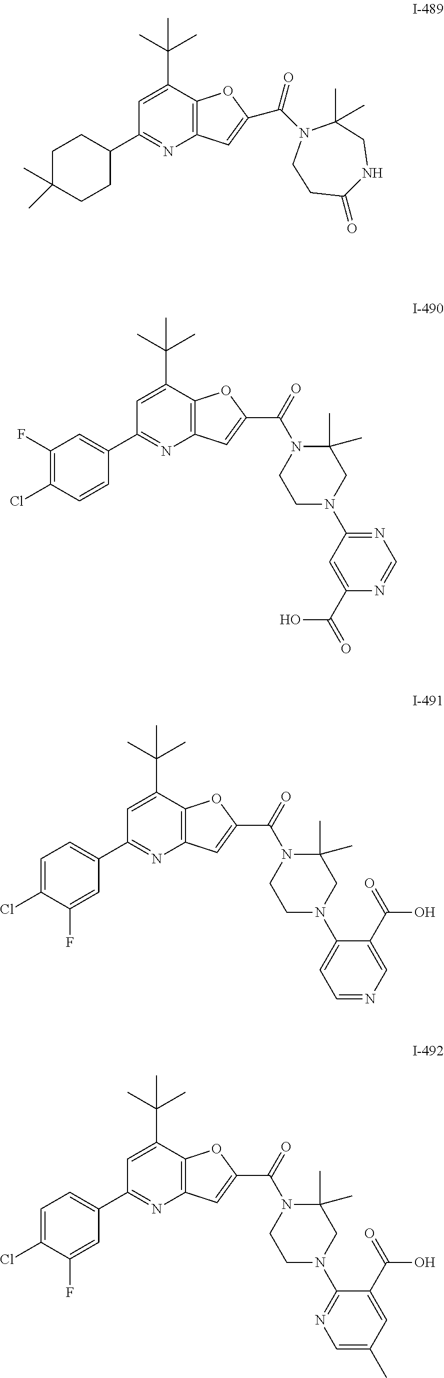 [SMILES: CC1(C)CCC(c2cc(C(C)(C)C)c3oc(C(=O)N4CCC(=O)NCC4(C)C)cc3n2)CC1 ; CC(C)(C)c1cc(-c2ccc(Cl)c(F)c2)nc2cc(C(=O)N3CCN(c4cc(C(=O)O)ncn4)CC3(C)C)oc12 ; CC(C)(C)c1cc(-c2ccc(Cl)c(F)c2)nc2cc(C(=O)N3CCN(c4ccncc4C(=O)O)CC3(C)C)oc12 ; Cc1cnc(N2CCN(C(=O)c3cc4nc(-c5ccc(Cl)c(F)c5)cc(C(C)(C)C)c4o3)C(C)(C)C2)c(C(=O)O)c1]

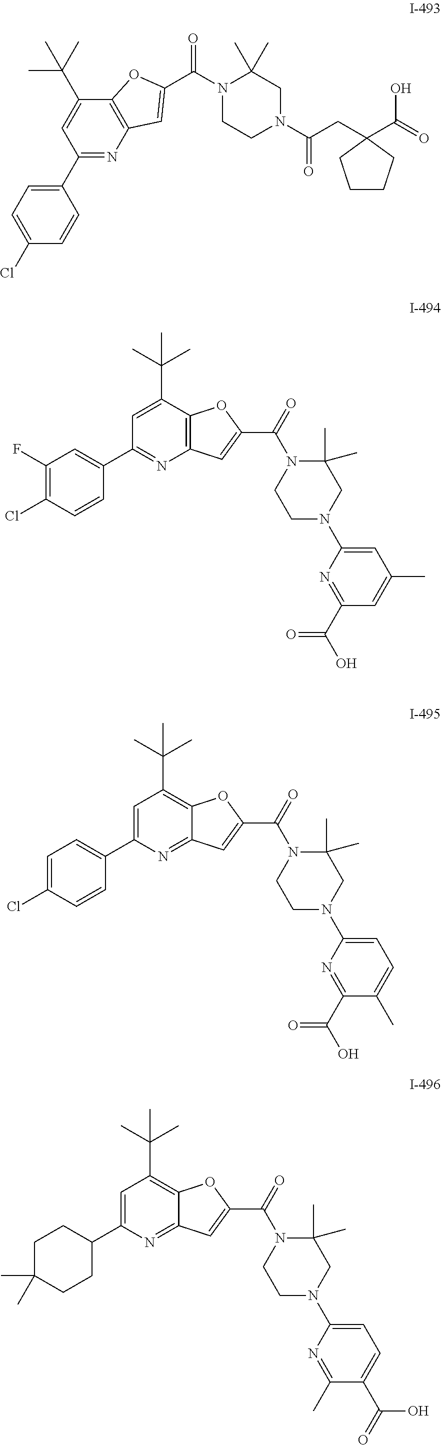

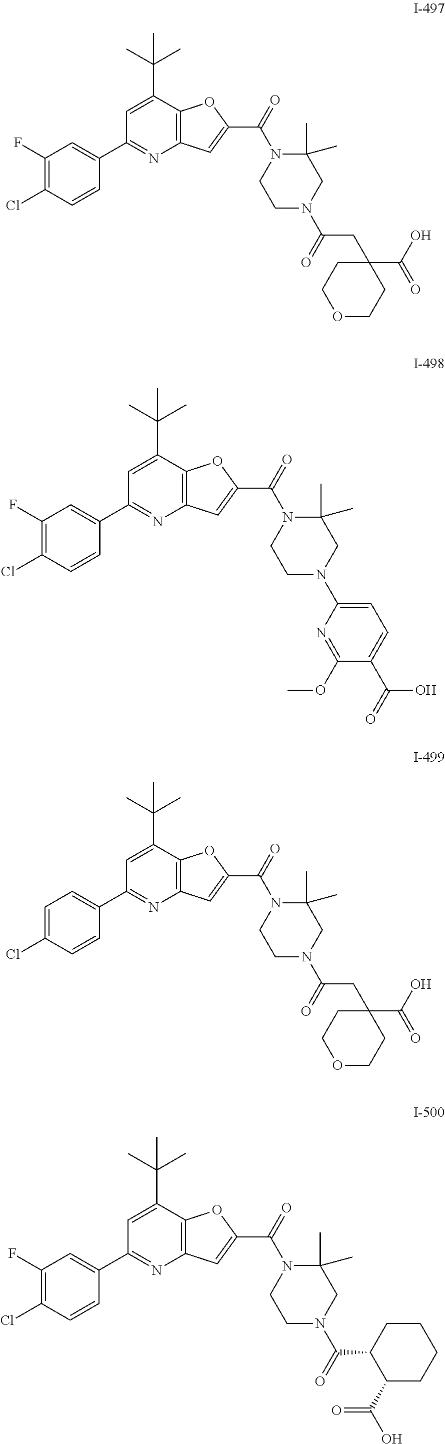 [SMILES: CC(C)(C)c1cc(-c2ccc(Cl)c(F)c2)nc2cc(C(=O)N3CCN(C(=O)CC4(C(=O)O)CCOCC4)CC3(C)C)oc12 ; COc1nc(N2CCN(C(=O)c3cc4nc(-c5ccc(Cl)c(F)c5)cc(C(C)(C)C)c4o3)C(C)(C)C2)ccc1C(=O)O ; CC(C)(C)c1cc(-c2ccc(Cl)cc2)nc2cc(C(=O)N3CCN(C(=O)CC4(C(=O)O)CCOCC4)CC3(C)C)oc12 ; CC(C)(C)c1cc(-c2ccc(Cl)c(F)c2)nc2cc(C(=O)N3CCN(C(=O)[C@@H]4CCCC[C@@H]4C(=O)O)CC3(C)C)oc12]

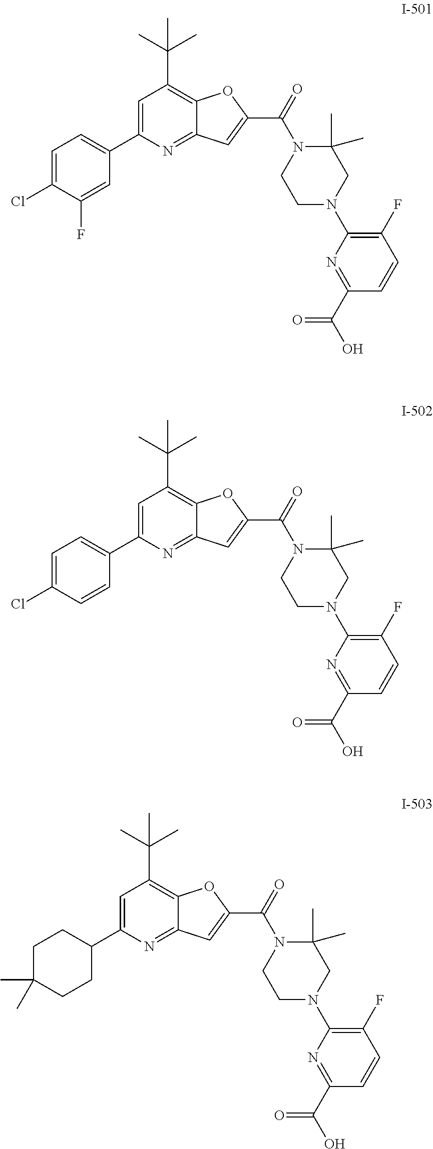 [SMILES: CC(C)(C)c1cc(-c2ccc(Cl)c(F)c2)nc2cc(C(=O)N3CCN(c4nc(C(=O)O)ccc4F)CC3(C)C)oc12 ; CC(C)(C)c1cc(-c2ccc(Cl)cc2)nc2cc(C(=O)N3CCN(c4nc(C(=O)O)ccc4F)CC3(C)C)oc12 ; CC1(C)CCC(c2cc(C(C)(C)C)c3oc(C(=O)N4CCN(c5nc(C(=O)O)ccc5F)CC4(C)C)cc3n2)CC1]

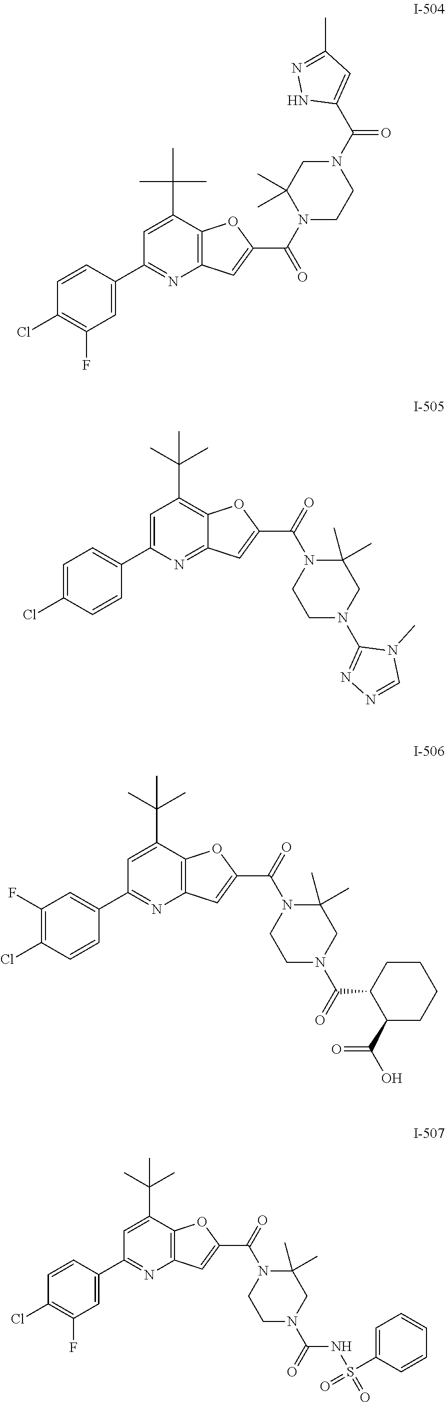 [SMILES: Cc1cc(C(=O)N2CCN(C(=O)c3cc4nc(-c5ccc(Cl)c(F)c5)cc(C(C)(C)C)c4o3)C(C)(C)C2)[nH]n1 ; Cn1cnnc1N1CCN(C(=O)c2cc3nc(-c4ccc(Cl)cc4)cc(C(C)(C)C)c3o2)C(C)(C)C1 ; CC(C)(C)c1cc(-c2ccc(Cl)c(F)c2)nc2cc(C(=O)N3CCN(C(=O)[C@@H]4CCCC[C@H]4C(=O)O)CC3(C)C)oc12 ; CC(C)(C)c1cc(-c2ccc(Cl)c(F)c2)nc2cc(C(=O)N3CCN(C(=O)NS(=O)(=O)c4ccccc4)CC3(C)C)oc12]

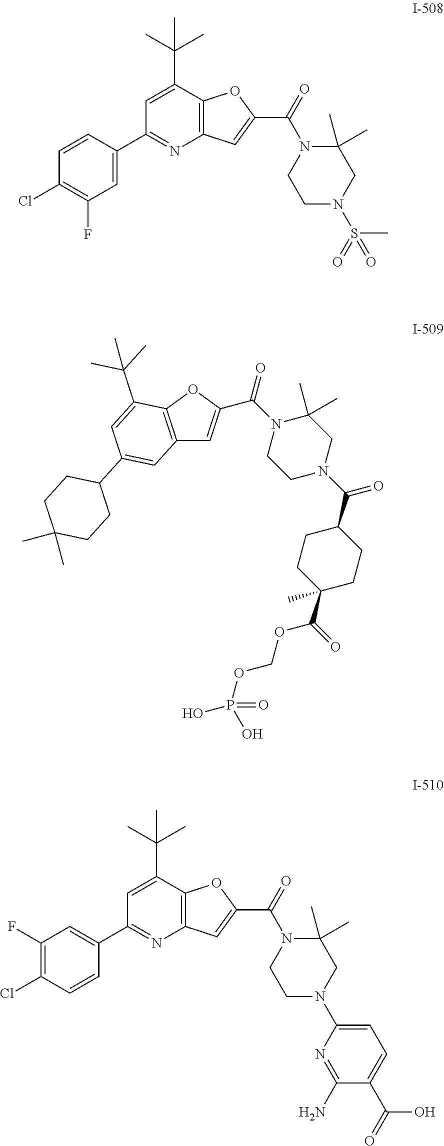

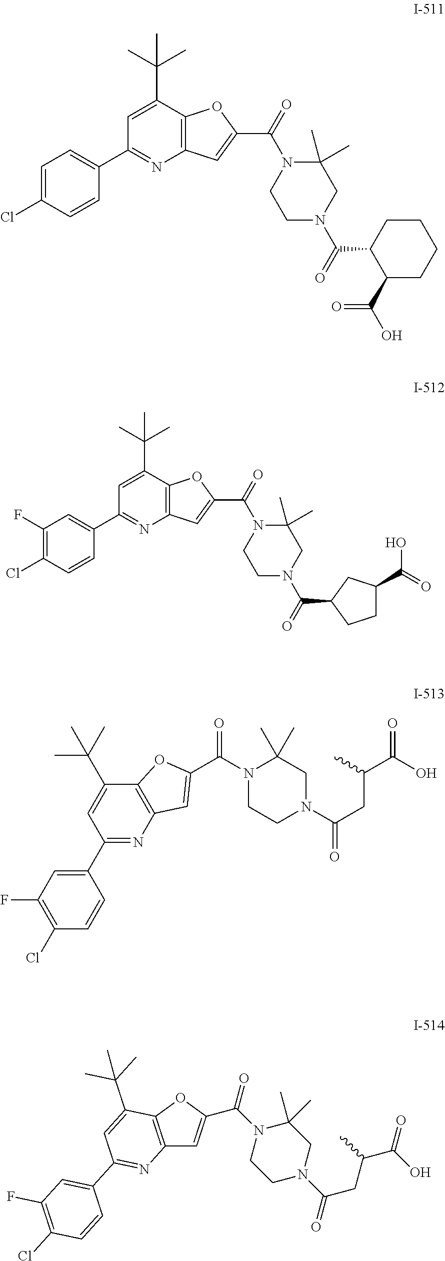

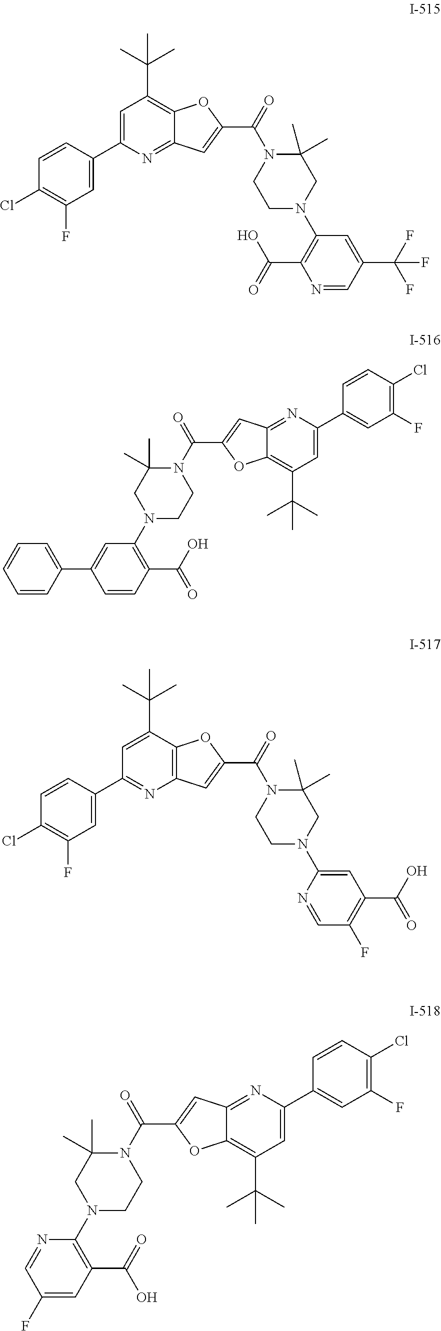 [SMILES: CC(C)(C)c1cc(-c2ccc(Cl)c(F)c2)nc2cc(C(=O)N3CCN(c4cc(C(F)(F)F)cnc4C(=O)O)CC3(C)C)oc12 ; CC(C)(C)c1cc(-c2ccc(Cl)c(F)c2)nc2cc(C(=O)N3CCN(c4cc(-c5ccccc5)ccc4C(=O)O)CC3(C)C)oc12 ; CC(C)(C)c1cc(-c2ccc(Cl)c(F)c2)nc2cc(C(=O)N3CCN(c4cc(C(=O)O)c(F)cn4)CC3(C)C)oc12 ; CC(C)(C)c1cc(-c2ccc(Cl)c(F)c2)nc2cc(C(=O)N3CCN(c4ncc(F)cc4C(=O)O)CC3(C)C)oc12]

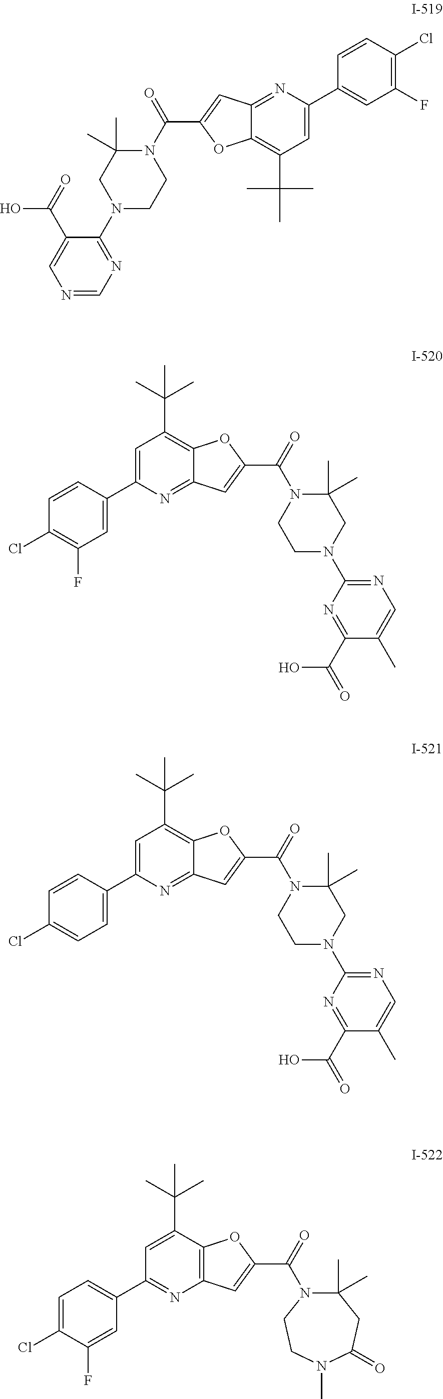 [SMILES: CC(C)(C)c1cc(-c2ccc(Cl)c(F)c2)nc2cc(C(=O)N3CCN(c4ncncc4C(=O)O)CC3(C)C)oc12 ; Cc1cnc(N2CCN(C(=O)c3cc4nc(-c5ccc(Cl)c(F)c5)cc(C(C)(C)C)c4o3)C(C)(C)C2)nc1C(=O)O ; Cc1cnc(N2CCN(C(=O)c3cc4nc(-c5ccc(Cl)cc5)cc(C(C)(C)C)c4o3)C(C)(C)C2)nc1C(=O)O ; CN1CCN(C(=O)c2cc3nc(-c4ccc(Cl)c(F)c4)cc(C(C)(C)C)c3o2)C(C)(C)CC1=O]

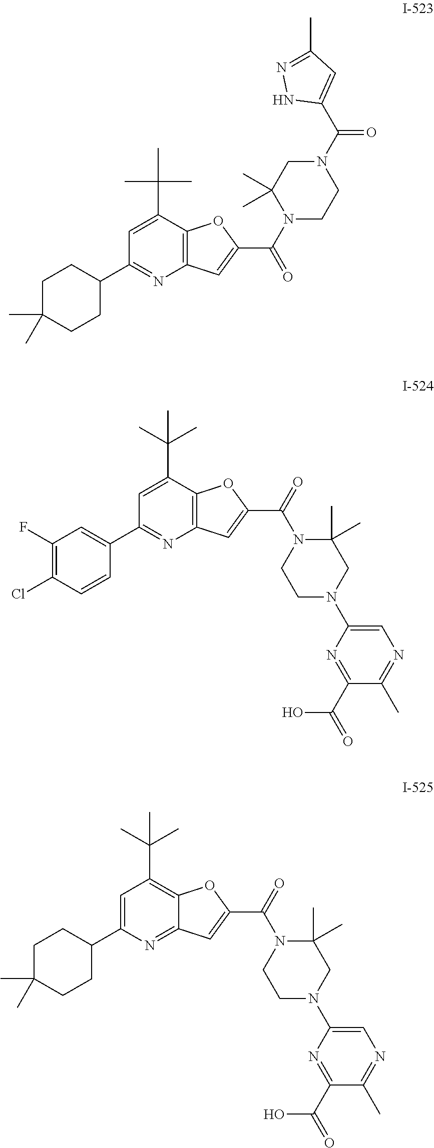

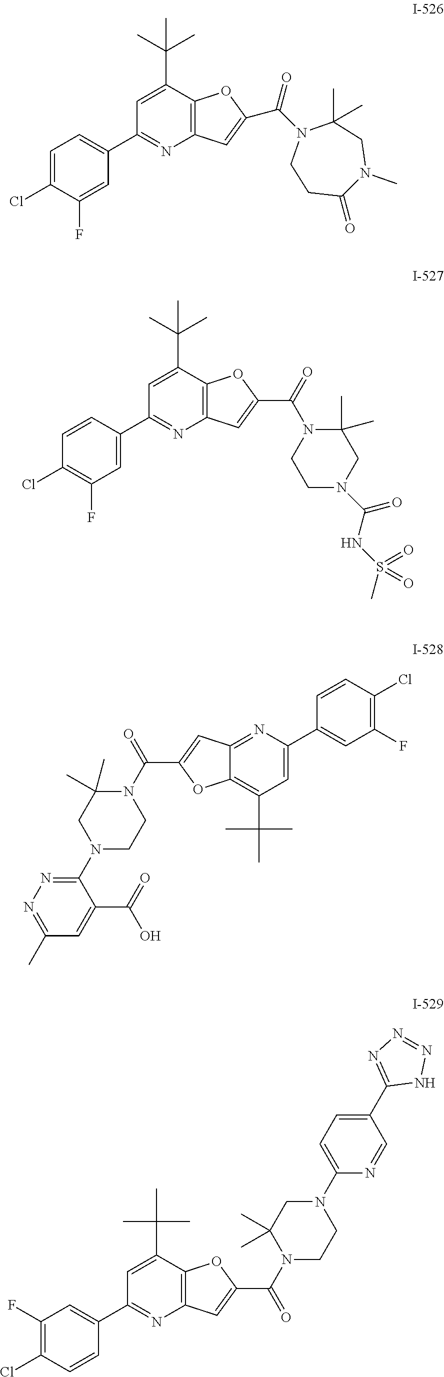 [SMILES: CN1CC(C)(C)N(C(=O)c2cc3nc(-c4ccc(Cl)c(F)c4)cc(C(C)(C)C)c3o2)CCC1=O ; CC(C)(C)c1cc(-c2ccc(Cl)c(F)c2)nc2cc(C(=O)N3CCN(C(=O)NS(C)(=O)=O)CC3(C)C)oc12 ; Cc1cc(C(=O)O)c(N2CCN(C(=O)c3cc4nc(-c5ccc(Cl)c(F)c5)cc(C(C)(C)C)c4o3)C(C)(C)C2)nn1 ; CC(C)(C)c1cc(-c2ccc(Cl)c(F)c2)nc2cc(C(=O)N3CCN(c4ccc(-c5nnn[nH]5)cn4)CC3(C)C)oc12]

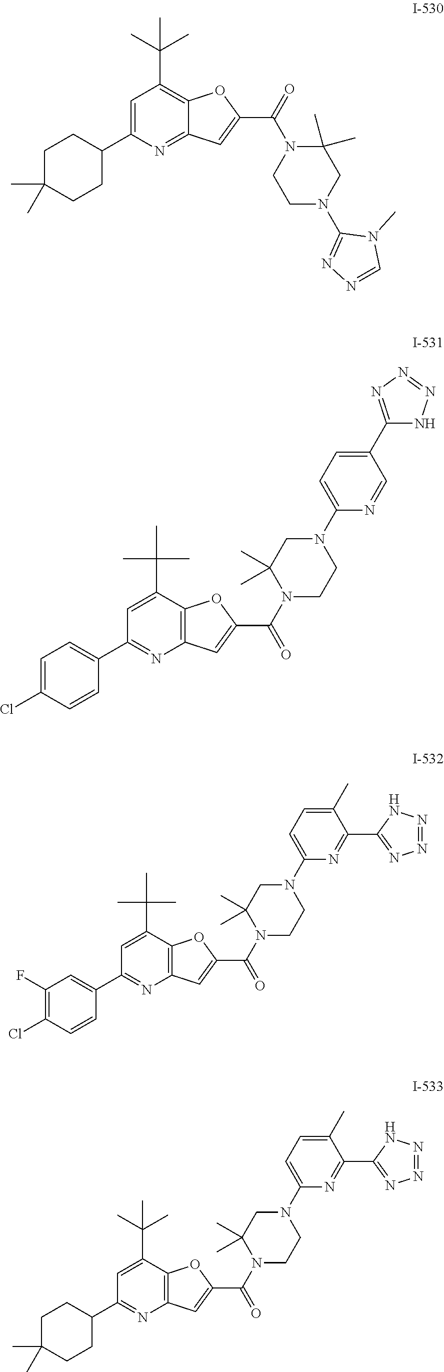

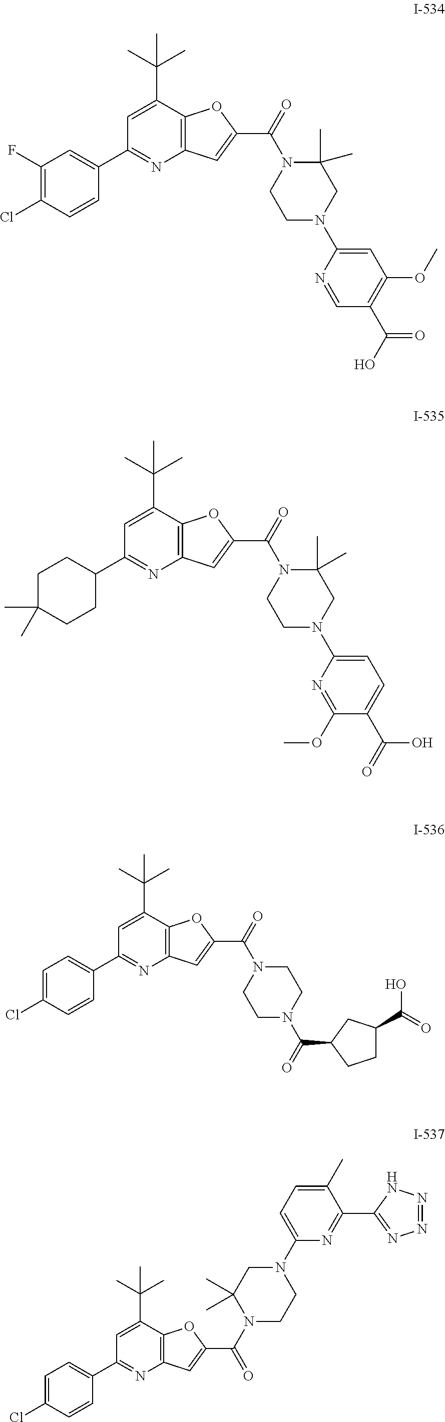

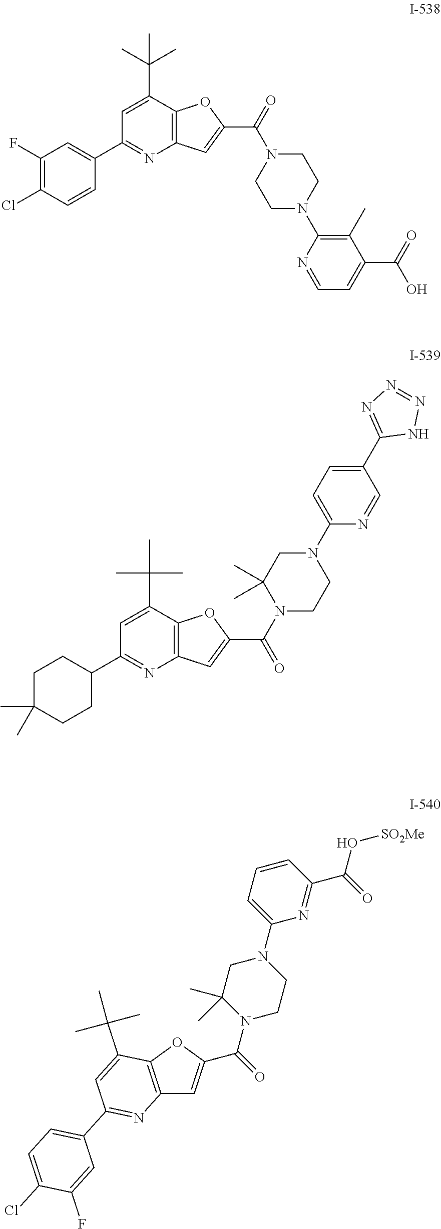 [SMILES: Cc1c(C(=O)O)ccnc1N1CCN(C(=O)c2cc3nc(-c4ccc(Cl)c(F)c4)cc(C(C)(C)C)c3o2)CC1 ; CC1(C)CCC(c2cc(C(C)(C)C)c3oc(C(=O)N4CCN(c5ccc(-c6nnn[nH]6)cn5)CC4(C)C)cc3n2)CC1 ; CC(C)(C)c1cc(-c2ccc(Cl)c(F)c2)nc2cc(C(=O)N3CCN(c4cccc(C(=O)OS(C)(=O)=O)n4)CC3(C)C)oc12]

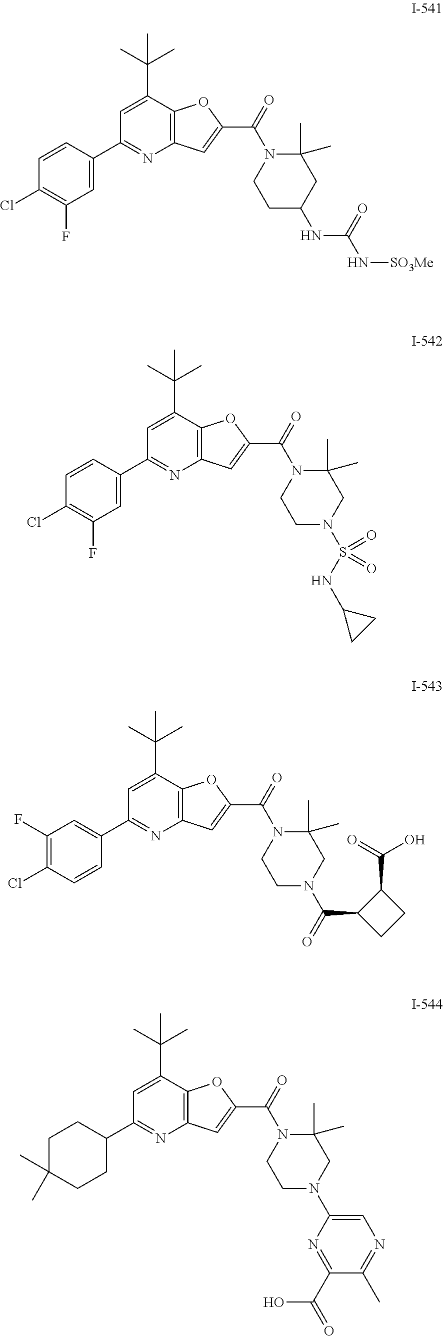

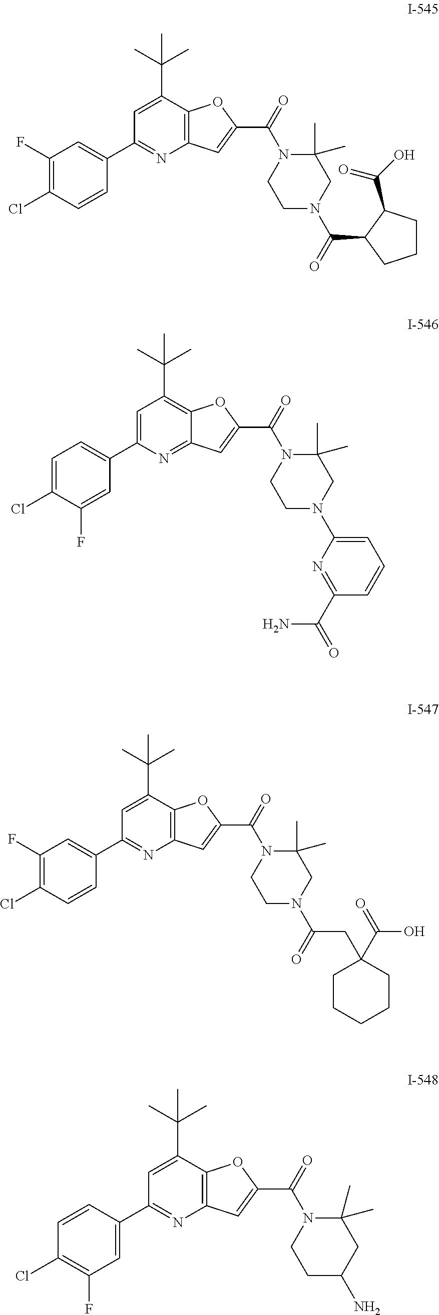 [SMILES: CC(C)(C)c1cc(-c2ccc(Cl)c(F)c2)nc2cc(C(=O)N3CCN(C(=O)[C@@H]4CCC[C@@H]4C(=O)O)CC3(C)C)oc12 ; CC(C)(C)c1cc(-c2ccc(Cl)c(F)c2)nc2cc(C(=O)N3CCN(c4cccc(C(N)=O)n4)CC3(C)C)oc12 ; CC(C)(C)c1cc(-c2ccc(Cl)c(F)c2)nc2cc(C(=O)N3CCN(C(=O)CC4(C(=O)O)CCCCC4)CC3(C)C)oc12 ; CC(C)(C)c1cc(-c2ccc(Cl)c(F)c2)nc2cc(C(=O)N3CCC(N)CC3(C)C)oc12]

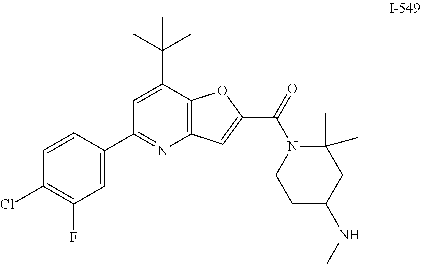 [SMILES: CNC1CCN(C(=O)c2cc3nc(-c4ccc(Cl)c(F)c4)cc(C(C)(C)C)c3o2)C(C)(C)C1]

In one embodiment, the invention are directed to the compounds represented by any one of the following structural formulae and pharmaceutically acceptable salts thereof:

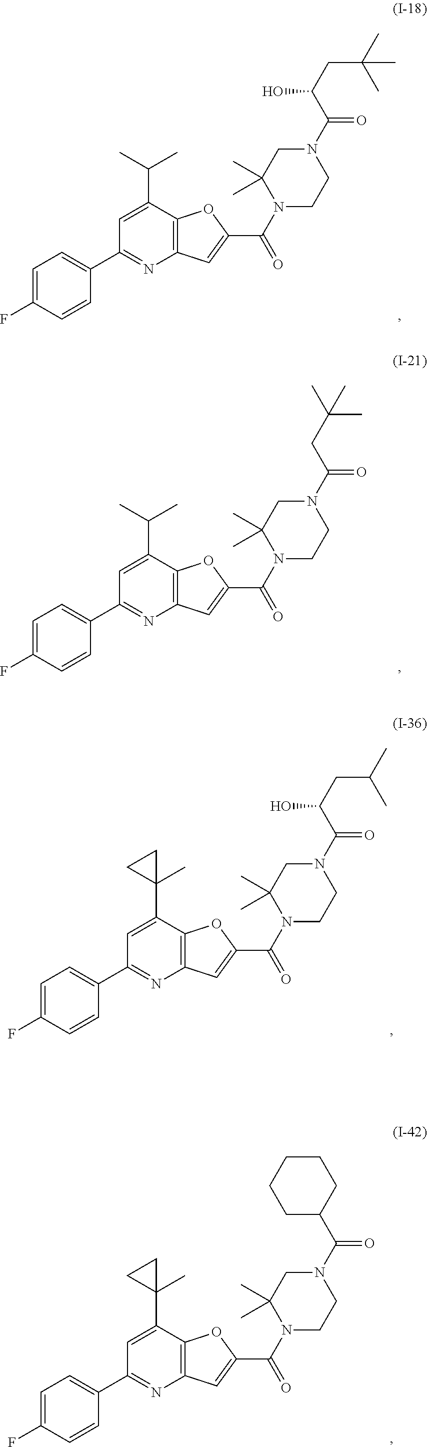 [SMILES: CC(C)c1cc(-c2ccc(F)cc2)nc2cc(C(=O)N3CCN(C(=O)[C@H](O)CC(C)(C)C)CC3(C)C)oc12 ; CC(C)c1cc(-c2ccc(F)cc2)nc2cc(C(=O)N3CCN(C(=O)CC(C)(C)C)CC3(C)C)oc12 ; CC(C)C[C@@H](O)C(=O)N1CCN(C(=O)c2cc3nc(-c4ccc(F)cc4)cc(C4(C)CC4)c3o2)C(C)(C)C1 ; CC1(c2cc(-c3ccc(F)cc3)nc3cc(C(=O)N4CCN(C(=O)C5CCCCC5)CC4(C)C)oc23)CC1]

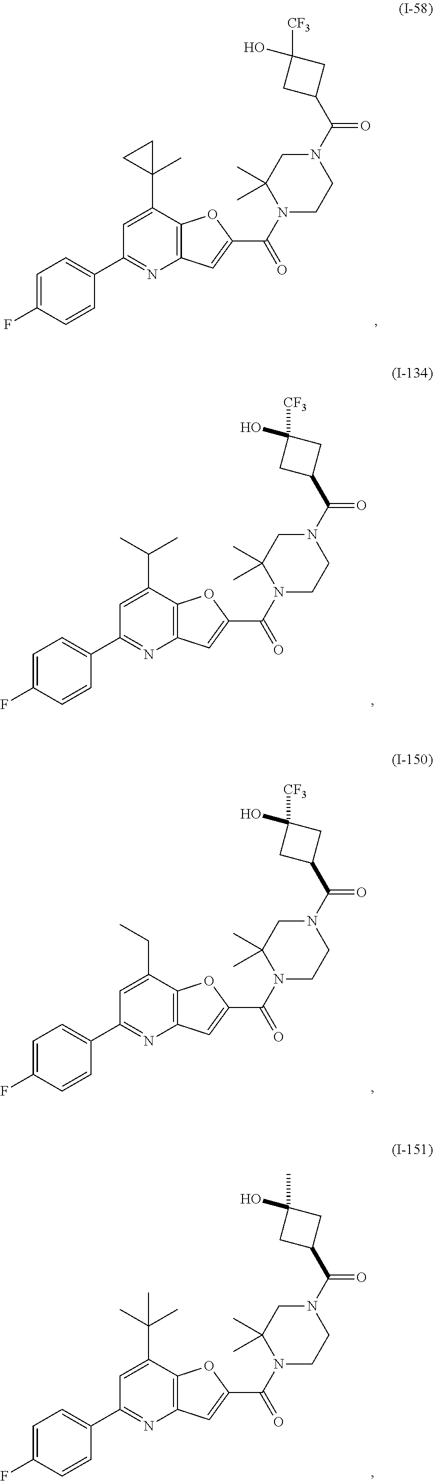

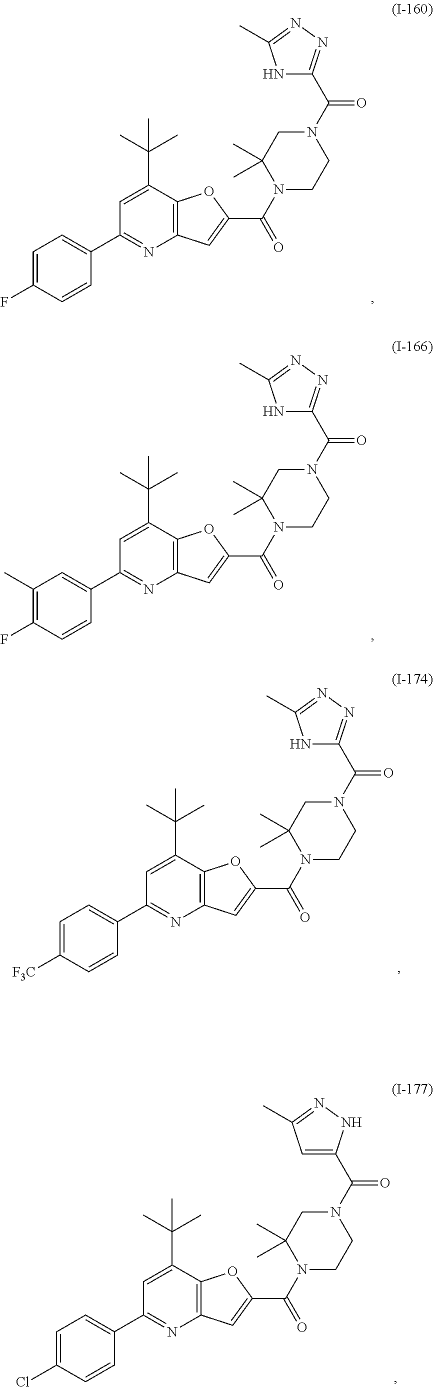 [SMILES: Cc1nnc(C(=O)N2CCN(C(=O)c3cc4nc(-c5ccc(F)cc5)cc(C(C)(C)C)c4o3)C(C)(C)C2)[nH]1 ; Cc1nnc(C(=O)N2CCN(C(=O)c3cc4nc(-c5ccc(F)c(C)c5)cc(C(C)(C)C)c4o3)C(C)(C)C2)[nH]1 ; Cc1nnc(C(=O)N2CCN(C(=O)c3cc4nc(-c5ccc(C(F)(F)F)cc5)cc(C(C)(C)C)c4o3)C(C)(C)C2)[nH]1 ; Cc1cc(C(=O)N2CCN(C(=O)c3cc4nc(-c5ccc(Cl)cc5)cc(C(C)(C)C)c4o3)C(C)(C)C2)[nH]n1]

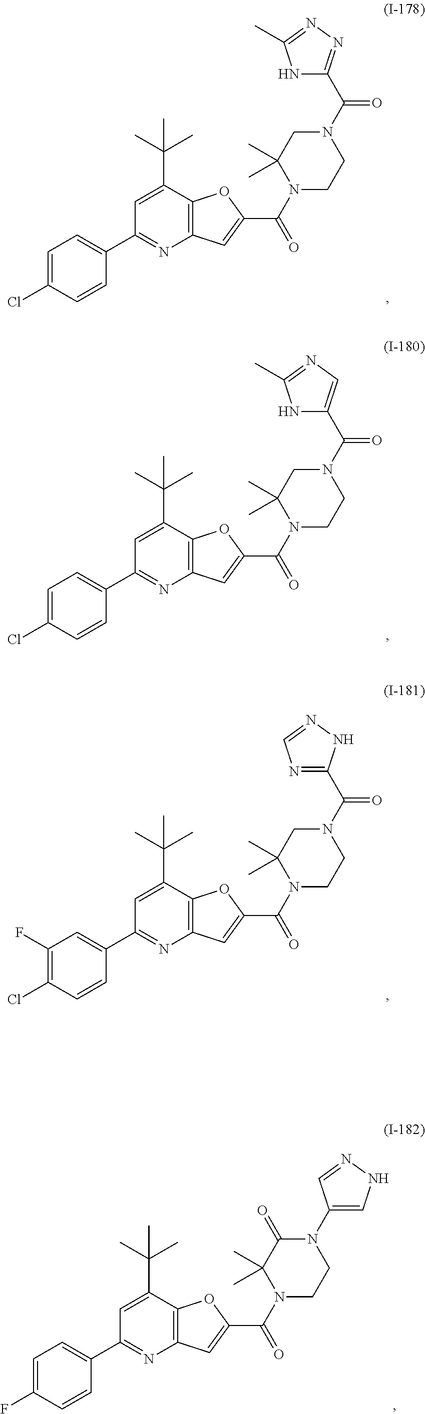 [SMILES: Cc1nnc(C(=O)N2CCN(C(=O)c3cc4nc(-c5ccc(Cl)cc5)cc(C(C)(C)C)c4o3)C(C)(C)C2)[nH]1 ; Cc1ncc(C(=O)N2CCN(C(=O)c3cc4nc(-c5ccc(Cl)cc5)cc(C(C)(C)C)c4o3)C(C)(C)C2)[nH]1 ; CC(C)(C)c1cc(-c2ccc(Cl)c(F)c2)nc2cc(C(=O)N3CCN(C(=O)c4ncn[nH]4)CC3(C)C)oc12 ; CC(C)(C)c1cc(-c2ccc(F)cc2)nc2cc(C(=O)N3CCN(c4cn[nH]c4)C(=O)C3(C)C)oc12]

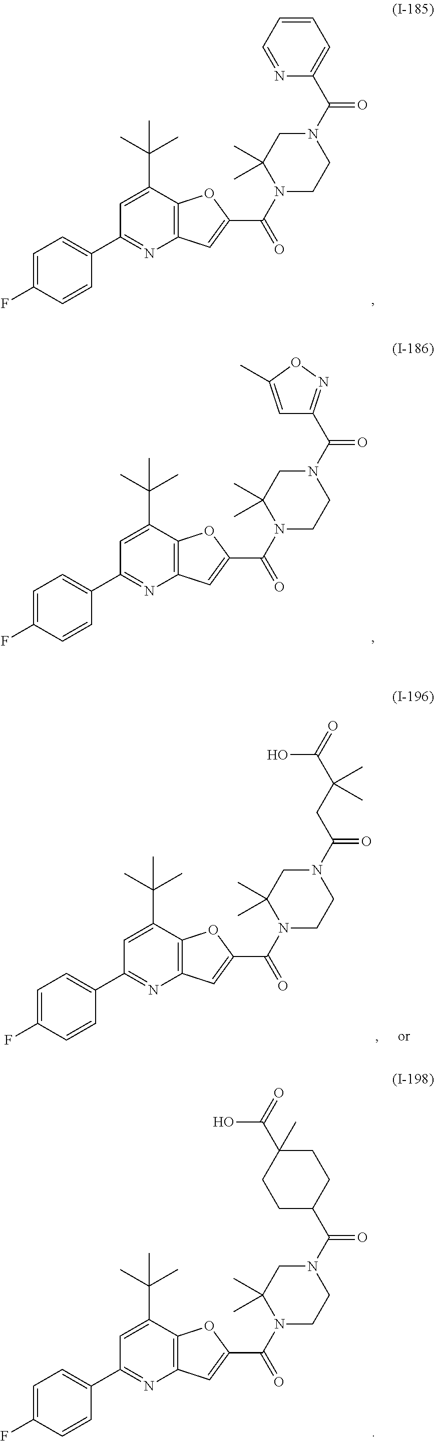 [SMILES: CC(C)(C)c1cc(-c2ccc(F)cc2)nc2cc(C(=O)N3CCN(C(=O)c4ccccn4)CC3(C)C)oc12 ; Cc1cc(C(=O)N2CCN(C(=O)c3cc4nc(-c5ccc(F)cc5)cc(C(C)(C)C)c4o3)C(C)(C)C2)no1 ; CC(C)(CC(=O)N1CCN(C(=O)c2cc3nc(-c4ccc(F)cc4)cc(C(C)(C)C)c3o2)C(C)(C)C1)C(=O)O ; CC1(C(=O)O)CCC(C(=O)N2CCN(C(=O)c3cc4nc(-c5ccc(F)cc5)cc(C(C)(C)C)c4o3)C(C)(C)C2)CC1]

In another embodiment, the invention are directed to the compounds represented by any one of the following structural formulae and pharmaceutically acceptable salts thereof:

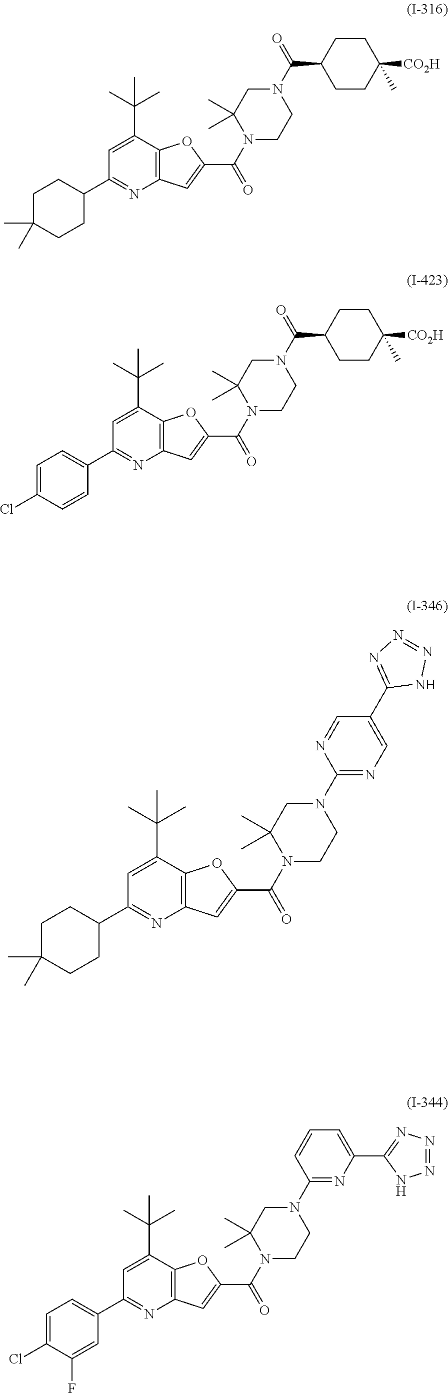 [SMILES: CC1(C)CCC(c2cc(C(C)(C)C)c3oc(C(=O)N4CCN(C(=O)[C@H]5CC[C@@](C)(C(=O)O)CC5)CC4(C)C)cc3n2)CC1 ; CC(C)(C)c1cc(-c2ccc(Cl)cc2)nc2cc(C(=O)N3CCN(C(=O)[C@H]4CC[C@@](C)(C(=O)O)CC4)CC3(C)C)oc12 ; CC1(C)CCC(c2cc(C(C)(C)C)c3oc(C(=O)N4CCN(c5ncc(-c6nnn[nH]6)cn5)CC4(C)C)cc3n2)CC1 ; CC(C)(C)c1cc(-c2ccc(Cl)c(F)c2)nc2cc(C(=O)N3CCN(c4cccc(-c5nnn[nH]5)n4)CC3(C)C)oc12]

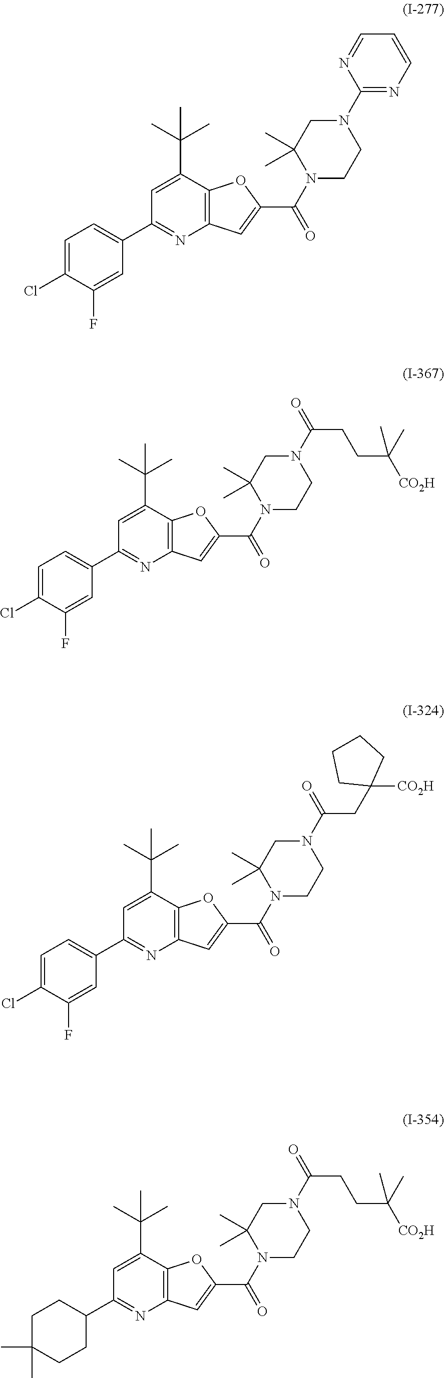 [SMILES: CC(C)(C)c1cc(-c2ccc(Cl)c(F)c2)nc2cc(C(=O)N3CCN(c4ncccn4)CC3(C)C)oc12 ; CC(C)(CCC(=O)N1CCN(C(=O)c2cc3nc(-c4ccc(Cl)c(F)c4)cc(C(C)(C)C)c3o2)C(C)(C)C1)C(=O)O ; CC(C)(C)c1cc(-c2ccc(Cl)c(F)c2)nc2cc(C(=O)N3CCN(C(=O)CC4(C(=O)O)CCCC4)CC3(C)C)oc12 ; CC1(C)CCC(c2cc(C(C)(C)C)c3oc(C(=O)N4CCN(C(=O)CCC(C)(C)C(=O)O)CC4(C)C)cc3n2)CC1]

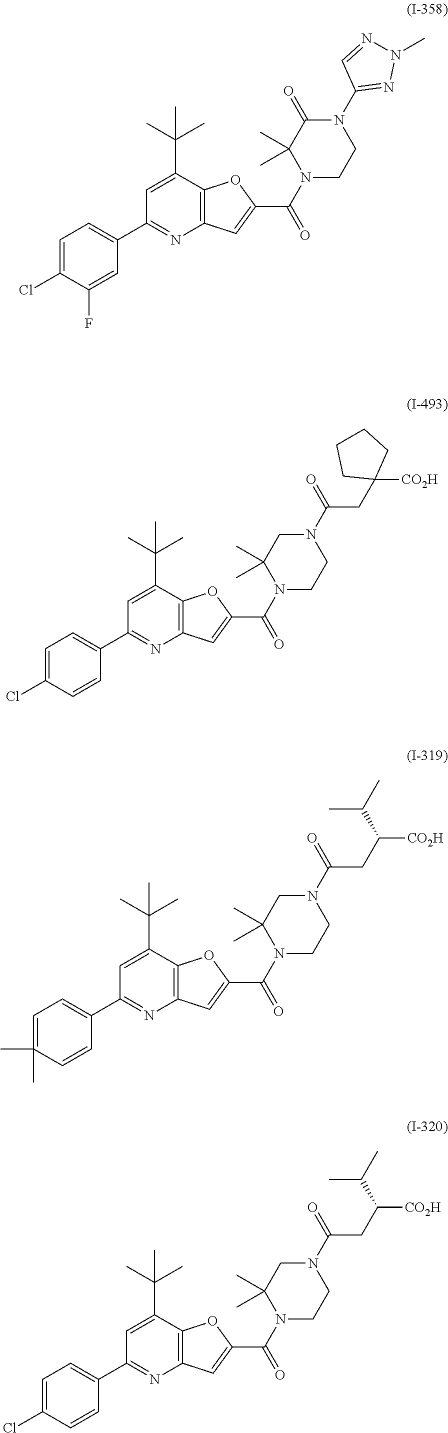

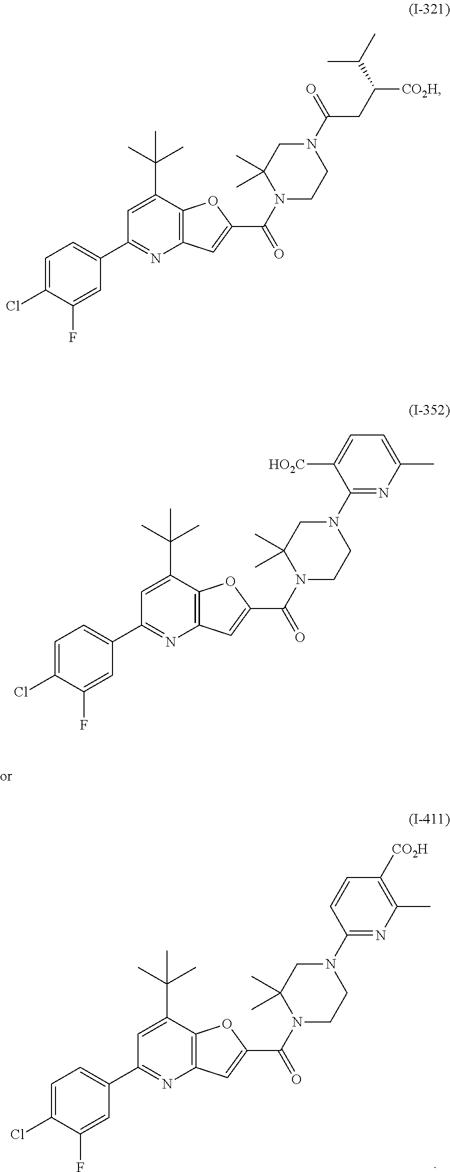 [SMILES: CC(C)[C@@H](CC(=O)N1CCN(C(=O)c2cc3nc(-c4ccc(Cl)c(F)c4)cc(C(C)(C)C)c3o2)C(C)(C)C1)C(=O)O ; Cc1ccc(C(=O)O)c(N2CCN(C(=O)c3cc4nc(-c5ccc(Cl)c(F)c5)cc(C(C)(C)C)c4o3)C(C)(C)C2)n1 ; Cc1nc(N2CCN(C(=O)c3cc4nc(-c5ccc(Cl)c(F)c5)cc(C(C)(C)C)c4o3)C(C)(C)C2)ccc1C(=O)O]

In some embodiments, the variables of formulae (I), (II), and (III) are each and independently as depicted in the compounds of the disclosure including the specific compounds depicted above.

In general, the compounds of the invention can be prepared by methods described herein or by other methods known to those skilled in the art. Specific exemplary preparations of the compounds of the invention are described in the Exemplification section below.

In one embodiment, the methods of preparing compounds represented by Formula (I) or pharmaceutically acceptable salts thereof employ the step of reacting Compound (X-1) with Compound (Y-1) under suitable conditions to form a compound represented by formula (I) or a pharmaceutically acceptable salt thereof:

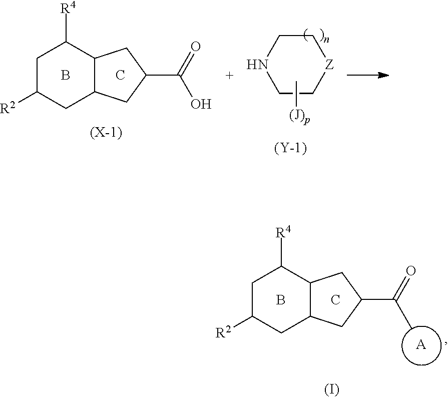

wherein the variables of Compound (X-1) with Compound (Y-1) are each and independently as described above for formula (I). Any suitable conditions known in the art to effectuate the reaction, such as those for amidations, can be used. Specific suitable conditions are as described in the Exemplification section below.

In another embodiment, the methods of preparing compounds represented by Formula (I) or pharmaceutically acceptable salts thereof employ the step of reacting Compound (X-2) with X-L¹ under suitable conditions to form a compound of formula (I) or a pharmaceutically acceptable salt thereof, wherein A is

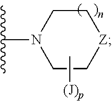

Z is —NX—; and the remaining variables of formula (I) are each and independently as described above:

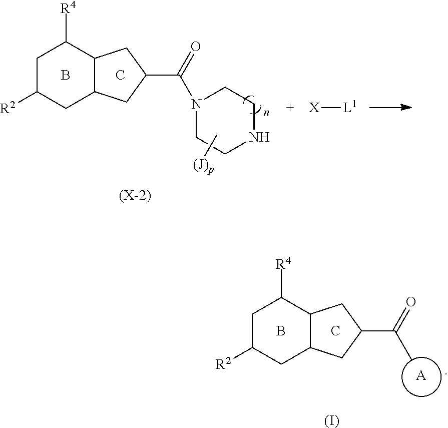

X of X-L¹ is as described above for formula (I) and L¹ of X-L¹ is halogen (e.g., —Cl) or —OH, and the variables of Compound (X-2) are each and independently as described for above Formula (I). Any suitable conditions known in the art to effectuate the reaction, such as those for carbon-nitrogen coupling reactions (e.g., nucleophilic substitution, amidation, etc.), can be used. Specific suitable conditions are as described in the Exemplification section below.

In another embodiment, the methods of preparing compounds represented by Formula (I) or pharmaceutically acceptable salts thereof employ the step of reacting Compound (X-3) with R²-L³ under suitable conditions to form a compound of formula (I) or a pharmaceutically acceptable salt thereof:

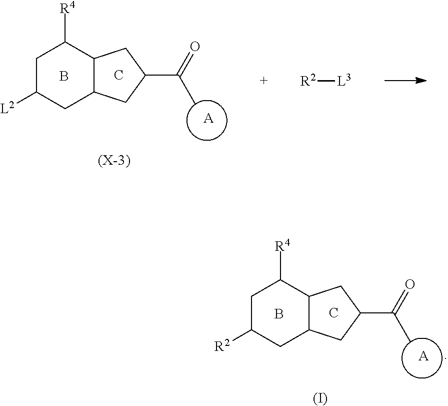

L² of Compound (X-3) is halogen (e.g., —Cl), and the remaining variables of Compound (X-3) are each and independently as described above for formula (I), and wherein L³ of R²-L³ is —B(OR^{a})₂, wherein R^{a} is —H or two R^{a} together with the atom to which they are attached form a dioxaborolane optionally substituted with C₁₋₂alkyl, and R² of R²-L³ is as described above for formula (I). In one specific embodiment, two R^{a} together with the atom to which they are attached form 4,4,5,5-tetramethyl-1,3,2-dioxaborolane. Any suitable conditions known in the art to effectuate the reaction, such as those for Suzuki coupling reactions, can be used. Specific suitable conditions are as described in the Exemplification section below.

Compounds of this invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

As described herein, a specified number range of atoms includes any integer therein. For example, a group having from 1-4 atoms could have 1, 2, 3, or 4 atoms.

As described herein, compounds of the invention may optionally be substituted with one or more substituents, such as are illustrated generally herein, or as exemplified by particular classes, subclasses, and species of the invention. It will be appreciated that the phrase “optionally substituted” is used interchangeably with the phrase “substituted or unsubstituted.” In general, the term “substituted”, whether preceded by the term “optionally” or not, refers to the replacement of hydrogen radicals in a given structure with the radical of a specified substituent. Unless otherwise indicated, an optionally substituted group may have a substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds.

Unless otherwise indicated, a substituent connected by a bond drawn from the center of a ring means that the substituent can be bonded to any position in the ring. In example i below, for instance, J¹ can be bonded to any position on the pyridyl ring. For bicyclic rings, a bond drawn through both rings indicates that the substituent can be bonded from any position of the bicyclic ring. In example ii below, for instance, J¹ can be bonded to the 5-membered ring (on the nitrogen atom, for instance), and to the 6-membered ring.

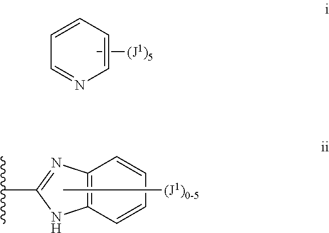

The term “stable”, as used herein, refers to compounds that are not substantially altered when subjected to conditions to allow for their production, detection, recovery, purification, and use for one or more of the purposes disclosed herein. In some embodiments, a stable compound or chemically feasible compound is one that is not substantially altered when kept at a temperature of 40° C., or less, in the absence of moisture or other chemically reactive conditions, for at least a week.

The term “aliphatic” or “aliphatic group”, as used herein, means a straight-chain (i.e., unbranched) or branched, substituted or unsubstituted, hydrocarbon chain that is completely saturated or that contains one or more units of unsaturation that has a single point of attachment to the rest of the molecule.

Unless otherwise specified, aliphatic groups contain 1-20 aliphatic carbon atoms. In some embodiments, aliphatic groups contain 1-10 aliphatic carbon atoms. In other embodiments, aliphatic groups contain 1-8 aliphatic carbon atoms. In still other embodiments, aliphatic groups contain 1-6 aliphatic carbon atoms, and in yet other embodiments, aliphatic groups contain 1-4 aliphatic carbon atoms. Aliphatic groups may be linear or branched, substituted or unsubstituted alkyl, alkenyl, or alkynyl groups. Specific examples include, but are not limited to, methyl, ethyl, isopropyl, n-propyl, sec-butyl, vinyl, n-butenyl, ethynyl, and tert-butyl.

The term “cycloaliphatic” (or “carbocycle” or “carbocyclyl”) refers to a monocyclic C₃-C₈ hydrocarbon or bicyclic C₈-C₁₂ hydrocarbon that is completely saturated or that contains one or more units of unsaturation, but which is not aromatic, that has a single point of attachment to the rest of the molecule wherein any individual ring in said bicyclic ring system has 3-7 members. Examples of cycloaliphatic groups include, but are not limited to, cycloalkyl and cycloalkenyl groups. Specific examples include, but are not limited to, cyclohexyl, cyclopropenyl, and cyclobutyl.

The term “heterocycle”, “heterocyclyl”, or “heterocyclic” as used herein means non-aromatic, monocyclic, bicyclic, or tricyclic ring systems in which one or more ring members are an independently selected heteroatom. In some embodiments, the “heterocycle”, “heterocyclyl”, or “heterocyclic” group has three to fourteen ring members in which one or more ring members is a heteroatom independently selected from oxygen, sulfur, nitrogen, or phosphorus, and each ring in the system contains 3 to 7 ring members.

Examples of heterocycles include, but are not limited to, 3-1H-benzimidazol-2-one, 3-(1-alkyl)-benzimidazol-2-one, 2-tetrahydrofuranyl, 3-tetrahydrofuranyl, 2-tetrahydrothiophenyl, 3-tetrahydrothiophenyl, 2-morpholino, 3-morpholino, 4-morpholino, 2-thiomorpholino, 3-thiomorpholino, 4-thiomorpholino, 1-pyrrolidinyl, 2-pyrrolidinyl, 3-pyrrolidinyl, 1-tetrahydropiperazinyl, 2-tetrahydropiperazinyl, 3-tetrahydropiperazinyl, 1-piperidinyl, 2-piperidinyl, 3-piperidinyl, 1-pyrazolinyl, 3-pyrazolinyl, 4-pyrazolinyl, 5-pyrazolinyl, 1-piperidinyl, 2-piperidinyl, 3-piperidinyl, 4-piperidinyl, 2-thiazolidinyl, 3-thiazolidinyl, 4-thiazolidinyl, 1-imidazolidinyl, 2-imidazolidinyl, 4-imidazolidinyl, 5-imidazolidinyl, indolinyl, tetrahydroquinolinyl, tetrahydroisoquinolinyl, benzothiolane, benzodithiane, and 1,3-dihydro-imidazol-2-one.

Cyclic groups, (e.g. cycloaliphatic and heterocycles), can be linearly fused, bridged, or spirocyclic.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated”, as used herein, means that a moiety has one or more units of unsaturation. As would be known by one of skill in the art, unsaturated groups can be partially unsaturated or fully unsaturated. Examples of partially unsaturated groups include, but are not limited to, butene, cyclohexene, and tetrahydropyridine. Fully unsaturated groups can be aromatic, anti-aromatic, or non-aromatic. Examples of fully unsaturated groups include, but are not limited to, phenyl, cyclooctatetraene, pyridyl, thienyl, and 1-methylpyridin-2(1H)-one.

The term “alkoxy”, or “thioalkyl”, as used herein, refers to an alkyl group, as previously defined, attached through an oxygen (“alkoxy”) or sulfur (“thioalkyl”) atom.

The terms “haloalkyl” (e.g., haloC₁₋₄alkyl), “haloalkenyl”, “haloaliphatic”, and “haloalkoxy” mean alkyl, alkenyl or alkoxy, as the case may be, substituted with one or more halogen atoms. This term includes perfluorinated alkyl groups, such as —CF₃ and —CF₂CF₃.

The terms “halogen”, “halo”, and “hal” mean F, Cl, Br, or I.

The term “aryl” used alone or as part of a larger moiety as in “aralkyl”, “aralkoxy”, or “aryloxyalkyl”, refers to carbocyclic aromatic ring systems. The term includes monocyclic, bicyclic, and tricyclic ring systems having a total of five to fourteen ring members, wherein at least one ring in the system is aromatic and wherein each ring in the system contains 3 to 7 ring members. The term “aryl” may be used interchangeably with the term “aryl ring”.

The term “heteroaryl”, used alone or as part of a larger moiety as in “heteroaralkyl” or “heteroarylalkoxy”, refers to monocyclic, bicyclic, and tricyclic ring systems having a total of five to fourteen ring members, wherein at least one ring in the system is aromatic, at least one ring in the system contains one or more heteroatoms, and wherein each ring in the system contains 3 to 7 ring members. The term “heteroaryl” may be used interchangeably with the term “heteroaryl ring” or the term “heteroaromatic”. Examples of heteroaryl rings include, but are not limited to, 2-furanyl, 3-furanyl, N-imidazolyl, 2-imidazolyl, 4-imidazolyl, 5-imidazolyl, benzimidazolyl, 3-isoxazolyl, 4-isoxazolyl, 5-isoxazolyl, 2-oxazolyl, 4-oxazolyl, 5-oxazolyl, N-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, pyridazinyl (e.g., 3-pyridazinyl). 2-thiazolyl, 4-thiazolyl, 5-thiazolyl, tetrazolyl (e.g., 5-tetrazolyl), triazolyl (e.g., 2-triazolyl and 5-triazolyl), 2-thienyl. 3-thienyl, benzofuryl, benzothiophenyl, indolyl (e.g., 2-indolyl), pyrazolyl (e.g., 2-pyrazolyl), isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, purinyl, pyrazinyl, 1,3,5-triazinyl, quinolinyl (e.g., 2-quinolinyl, 3-quinolinyl, 4-quinolinyl), and isoquinolinyl (e.g., 1-isoquinolinyl, 3-isoquinolinyl, or 4-isoquinolinyl).

It shall be understood that the term “heteroaryl” includes certain types of heteroaryl rings that exist in equilibrium between two different forms. More specifically, for example, species such hydropyridine and pyridinone (and likewise hydroxypyrimidine and pyrimidinone) are meant to be encompassed within the definition of“heteroaryl.”

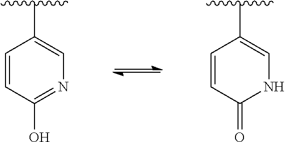 [SMILES: *c1ccc(O)nc1 ; *c1ccc(=O)[nH]c1]

The term “protecting group” and “protective group” as used herein, are interchangeable and refer to an agent used to temporarily block one or more desired functional groups in a compound with multiple reactive sites. In certain embodiments, a protecting group has one or more, or preferably all, of the following characteristics: a) is added selectively to a functional group in good yield to give a protected substrate that is b) stable to reactions occurring at one or more of the other reactive sites; and c) is selectively removable in good yield by reagents that do not attack the regenerated, deprotected functional group. As would be understood by one skilled in the art, in some cases, the reagents do not attack other reactive groups in the compound. In other cases, the reagents may also react with other reactive groups in the compound. Examples of protecting groups are detailed in Greene, T. W., Wuts, P. G in “Protective Groups in Organic Synthesis”, Third Edition, John Wiley & Sons, New York: 1999 (and other editions of the book), the entire contents of which are hereby incorporated by reference. The term “nitrogen protecting group”, as used herein, refers to an agent used to temporarily block one or more desired nitrogen reactive sites in a multifunctional compound. Preferred nitrogen protecting groups also possess the characteristics exemplified for a protecting group above, and certain exemplary nitrogen protecting groups are also detailed in Chapter 7 in Greene, T. W., Wuts, P. G in “Protective Groups in Organic Synthesis”, Third Edition, John Wiley & Sons, New York: 1999, the entire contents of which are hereby incorporated by reference.

In some embodiments, a methylene or carbon unit of an alkyl or aliphatic chain is optionally replaced with another atom or group. Examples of such atoms or groups include, but are not limited to, nitrogen, oxygen, sulfur, —C(O)—, —C(═N—CN)—, —C(═NR)—, —C(═NOR)—, —SO—, and —SO₂—. These atoms or groups can be combined to form larger groups. Examples of such larger groups include, but are not limited to, —OC(O)—, —C(O)CO—, —CO₂—, —C(O)NR—, —C(═N—CN), —NRCO—, —NRC(O)O—, —SO₂NR—, —NRSO₂—, —NRC(O)NR—, —OC(O)NR—, and —NRSO₂NR—, wherein R is, for example, H or C₁₋₆aliphatic. It should be understood that these groups can be bonded to the methylene or carbon units of the aliphatic chain via single, double, or triple bonds. An example of an optional replacement (nitrogen atom in this case) that is bonded to the aliphatic chain via a double bond would be —CH₂CH═N—CH₃. In some cases, especially on the terminal end, an optional replacement can be bonded to the aliphatic group via a triple bond. One example of this would be CH₂CH₂CH₂C≡N. It should be understood that in this situation, the terminal nitrogen is not bonded to another atom.

It should also be understood that, the term “methylene unit” or “carbon unit” can also refer to branched or substituted methylene or carbon units. For example, in an isopropyl moiety [—CH(CH₃)₂], a nitrogen atom (e.g. NR) replacing the first recited “methylene unit” would result in dimethylamine [—N(CH₃)₂]. In instances such as these, one of skill in the art would understand that the nitrogen atom will not have any additional atoms bonded to it, and the “R” from “NR” would be absent in this case.

Unless otherwise indicated, the optional replacements form a chemically stable compound. Optional replacements can occur both within the chain and/or at either end of the chain; i.e. both at the point of attachment and/or also at the terminal end. Two optional replacements can also be adjacent to each other within a chain so long as it results in a chemically stable compound. For example, a C₃ aliphatic can be optionally replaced by 2 nitrogen atoms to form —C—N≡N. The optional replacements can also completely replace all of the carbon atoms in a chain. For example, a C₃ aliphatic can be optionally replaced by —NR—, —C(O)—, and —NR— to form —NRC(O)NR— (a urea).

Unless otherwise indicated, if the replacement occurs at the terminal end, the replacement atom is bound to a hydrogen atom on the terminal end. For example, if a methylene unit of —CH₂CH₂CH₃ were optionally replaced with —O—, the resulting compound could be —OCH₂CH₃, —CH₂OCH₃, or —CH₂CH₂OH. It should be understood that if the terminal atom does not contain any free valence electrons, then a hydrogen atom is not required at the terminal end (e.g., —CH₂CH₂CH═O or —CH₂CH₂C≡N).

Unless otherwise indicated, structures depicted herein are also meant to include all isomeric (e.g., enantiomeric, diastereomeric, geometric, conformational, and rotational) forms of the structure. For example, the R and S configurations for each asymmetric center, (Z) and (E) double bond isomers, and (Z) and (E) conformational isomers are included in this invention. As would be understood to one skilled in the art, a substituent can freely rotate around any rotatable bonds. For example, a substituent drawn as

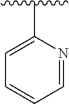 [SMILES: *c1ccccn1]

also represents.

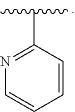 [SMILES: *c1ccccn1]

Therefore, single stereochemical isomers as well as enantiomeric, diastereomeric, geometric, conformational, and rotational mixtures of the present compounds are within the scope of the invention.

Unless otherwise indicated, all tautomeric forms of the compounds of the invention are within the scope of the invention.

Unless otherwise indicated, the term “compound of claim” in the Claims below is also meant to include a pharmaceutically acceptable salt thereof.

Unless otherwise indicated, when multiple recitations of a particular group (R⁵, R, X, J^{Y}, etc.) are made, the particular group in such multiple recitations is being defined independently, with or without “each” and/or “independently” terms. For example, each R for —(CR₂)ₜ—C(O)OR⁶ is being defined independently with or without “each” and/or “independently” terms being used in such definition. Also, when multiple substitutents and/or replacements are listed for a particular group, such substituents and/or replacements are made independently with or without “each” and/or “independently” terms. For example, the phrase “substituted with one or more J^{Y}” means substitution is being made independently for each J^{Y} with or without the terms “each” and/or “independently” being used.

In some aspects, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, for therapeutics and/or analytical tools or probes in biological assays. Especially deuterium (²H)-labelled compounds can also be used for therapeutic purposes.

In some embodiments, the invention is directed to isotope-labelled compounds of Structural Formula (I′) or pharmaceutically acceptable salts thereof, wherein the formula and variables of Structural Formula (I′) are each and independently as described above for Formula (I) or any other embodiments described above, provided that one or more atoms therein have been replaced by an atom or atoms having an atomic mass or mass number which differs from the atomic mass or mass number of the atom which usually occurs naturally (isotope labelled). Examples of isotopes which are commercially available and suitable for the invention include isotopes of hydrogen, carbon, nitrogen, oxygen, phosphorus, fluorine and chlorine, for example ²H, ³H, ¹³C, ¹⁴C, ¹⁵N, ¹⁸O, ¹⁷O, ³¹P, ³²P, ³⁵S, ¹⁸F and ³⁶Cl, respectively.

The isotope-labelled compounds of the invention, including pharmaceutically acceptable salts thereof, can be used in a number of beneficial ways. They can be suitable for medicaments and/or various types of assays, such as substrate tissue distribution assays. For example, tritium (³H)— and/or carbon-14 (¹⁴C)-labelled compounds are particularly useful for various types of assays, such as substrate tissue distribution assays, due to relatively simple preparation and excellent detectability. For example, deuterium (²H)-labelled compounds are therapeutically useful with potential therapeutic advantages over the non-²H-labelled compounds. In general, deuterium (²H)-labelled compounds can have higher metabolic stability as compared to those compounds that are not isotope-labelled owing to the kinetic isotope effect described below. Higher metabolic stability translates directly into an increased in vivo half-life or lower dosages, which under most circumstances would represent a preferred embodiment of the present invention. The isotope-labelled compounds of the invention can usually be prepared by carrying out the procedures disclosed in the synthesis schemes and the related description, in the example part and in the preparation part in the present text, replacing a non-isotope-labelled reactant by a readily available isotope-labelled reactant.

In some embodiments, the isotope-labelled compounds of the invention are deuterium (²H)-labelled compounds. In some specific embodiments, the invention is directed to deuterium (²H)-labelled compounds of Structural Formula (I′) or pharmaceutically acceptable salts thereof, wherein the formula and variables of Structural Formula (I′) are each and independently as described above for Formula (I) or any other embodiments described above, wherein one or more hydrogen atoms therein have been replaced by deuterium. In a further specific embodiment, up to six (e.g., one, two, three, four, five, or six) hydrogen atoms are replaced by deuterium. In some specific embodiments, the invention is directed to deuterium (²H)-labelled compounds of Structural Formula (I′) or pharmaceutically acceptable salts thereof, wherein Ring B—C is

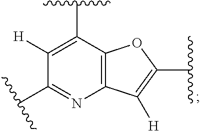 [SMILES: *c1cc(*)c2oc(*)cc2n1]

### Ring A is

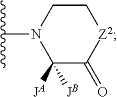

both J^{A} and J^{B} are methyl, and Z² is CD₃, and the other variables are each and independently as described above for Formula (I) above.

Deuterium (²H)-labelled compounds of the invention can manipulate the oxidative metabolism of the compound by way of the primary kinetic isotope effect. The primary kinetic isotope effect is a change of the rate for a chemical reaction that results from exchange of isotopic nuclei, which in turn is caused by the change in ground state energies necessary for covalent bond formation after this isotopic exchange. Exchange of a heavier isotope usually results in a lowering of the ground state energy for a chemical bond and thus causes a reduction in the rate-limiting bond breakage. If the bond breakage occurs in or in the vicinity of a saddle-point region along the coordinate of a multi-product reaction, the product distribution ratios can be altered substantially. For explanation: if deuterium is bonded to a carbon atom at a non-exchangeable position, rate differences of k_{M/}k_{D}=2-7 are typical. If this rate difference is successfully applied to, for example, a compound of Formula (I′), the profile of this compound in vivo can be drastically modified and result in improved pharmacokinetic properties. For a further discussion, see S. L. Harbeson and R. D. Tung, Deuterium In Drug Discovery and Development, Ann. Rep. Med. Chem. 2011, 46, 403-417, incorporated in its entirety herein by reference.

The concentration of the isotope(s) (e.g., deuterium) incorporated into the isotope-labelled compounds of the invention may be defined by the isotopic enrichment factor. The term “isotopic enrichment factor” as used herein means the ratio between the isotopic abundance and the natural abundance of a specified isotope. In some embodiments, if a substituent in a compound of the invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

When discovering and developing therapeutic agents, the person skilled in the art attempts to optimize pharmacokinetic parameters while retaining desirable in vitro properties. It may be reasonable to assume that many compounds with poor pharmacokinetic profiles are susceptible to oxidative metabolism. In vitro liver microsomal assays currently available provide valuable information on the course of oxidative metabolism of this type, which in turn permits the rational design of the deuterium (²H)-labelled compounds of the invention which can have improved stability through resistance to such oxidative metabolism. Significant improvements in the pharmacokinetic profiles of such compounds can thereby be obtained, and can be expressed quantitatively in terms of increases in the in vivo half-life (t_{1/2}), concentration at maximum therapeutic effect (Cₘₐₓ), area under the dose response curve (AUC), and bioavailability; and in terms of reduced clearance, dose and materials costs.

The following is intended to illustrate the above: a deuterium (²H)-labelled compound of the invention, which has multiple potential sites of attack for oxidative metabolism, for example benzylic hydrogen atoms and hydrogen atoms bonded to a nitrogen atom, is prepared as a series of analogues in which various combinations of hydrogen atoms are replaced by deuterium atoms, so that some, most or all of these hydrogen atoms have been replaced by deuterium atoms. Half-life determinations enable favorable and accurate determination of the extent to which the improvement in resistance to oxidative metabolism has improved. In this way, it is determined that the half-life of the parent compound can be extended by up to 100% as the result of deuterium-hydrogen exchange of this type.

Deuterium-hydrogen exchange in a deuterium (²H)-labelled compound of the invention can also be used to achieve a favorable modification of the metabolite spectrum of the starting compound in order to diminish or eliminate undesired toxic metabolites. For example, if a toxic metabolite arises through oxidative carbon-hydrogen (C—H) bond cleavage, it can reasonably be assumed that the deuterated analogue will greatly diminish or eliminate production of the unwanted metabolite, even if the particular oxidation is not a rate-determining step. Further information on the state of the art with respect to deuterium-hydrogen exchange may be found, for example in Hanzlik et al., J. Org. Chem. 55, 3992-3997, 1990, Reider et al., J. Org. Chem. 52, 3326-3334, 1987, Foster, Adv. Drug Res. 14, 1-40, 1985, Gillette et al, Biochemistry 33(10) 2927-2937, 1994, and Jarman et al. Carcinogenesis 16(4), 683-688, 1993.

### Pharmaceutically Acceptable Salts

The compounds of this invention can exist in free form for treatment, or where appropriate, as a pharmaceutically acceptable salt.

A “pharmaceutically acceptable salt” means any non-toxic salt of a compound of this invention that, upon administration to a recipient, is capable of providing, either directly or indirectly, a compound of this invention or an inhibitorily active metabolite or residue thereof. As used herein, the term “inhibitorily active metabolite or residue thereof” means that a metabolite or residue thereof is also an inhibitors of the PAR-2 signaling pathway.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. These salts can be prepared in situ during the final isolation and purification of the compounds. Acid addition salts can be prepared by 1) reacting the purified compound in its free-based form with a suitable organic or inorganic acid and 2) isolating the salt thus formed.

Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, valerate salts, and the like.

Base addition salts can be prepared by 1) reacting the purified compound in its acid form with a suitable organic or inorganic base and 2) isolating the salt thus formed. Salts derived from appropriate bases include alkali metal (e.g., sodium, lithium, and potassium), alkaline earth metal (e.g., magnesium and calcium), ammonium and N⁺ (C₁₋₄alkyl)₄ salts. This invention also envisions the quaternization of any basic nitrogen-containing groups of the compounds disclosed herein. Water or oil-soluble or dispersible products may be obtained by such quaternization.

Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Other acids and bases, while not in themselves pharmaceutically acceptable, may be employed in the preparation of salts useful as intermediates in obtaining the compounds of the invention and their pharmaceutically acceptable acid or base addition salts.

### Pharmaceutically Acceptable Derivatives or Prodrugs

In addition to the compounds of this invention, pharmaceutically acceptable derivatives or prodrugs of the compounds of this invention may also be employed in compositions to treat or prevent the diseases, conditions and disorders. Specific examples are described below.

The compounds of this invention can also exist as pharmaceutically acceptable derivatives. A “pharmaceutically acceptable derivative” is an adduct or derivative which, upon administration to a patient in need, is capable of providing, directly or indirectly, a compound as otherwise described herein, or a metabolite or residue thereof. Examples of pharmaceutically acceptable derivatives include, but are not limited to, esters and salts of such esters.

A “pharmaceutically acceptable derivative or prodrug” means any pharmaceutically acceptable ester, salt of an ester or other derivative or salt thereof of a compound, of this invention which, upon administration to a recipient, is capable of providing, either directly or indirectly, a compound of this invention or an inhibitorily active metabolite or residue thereof. Particularly favoured derivatives or prodrugs are those that increase the bioavailability of the compounds of this invention when such compounds are administered to a patient (e.g., by allowing an orally administered compound to be more readily absorbed into the blood) or which enhance delivery of the parent compound to a biological compartment (e.g., the brain or lymphatic system) relative to the parent species.

Pharmaceutically acceptable prodrugs of the compounds of this invention include, without limitation, esters, amino acid esters, phosphate esters, metal salts and sulfonate esters.

### Pharmaceutical Compositions

The present invention also provides compounds and compositions that are useful as inhibitors of the PAR-2 signaling pathway.

One aspect of this invention provides pharmaceutically acceptable compositions that comprise any of the compounds as described herein, and optionally comprise a pharmaceutically acceptable carrier, adjuvant or excipient.

The pharmaceutically acceptable carrier, adjuvant, or excipient, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention.

Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch: cellulose and its derivatives such as sodium carboxymethyl cellulose, ethyl cellulose and cellulose acetate: powdered tragacanth; malt; gelatin: talc; excipients such as cocoa butter and suppository waxes; oils such as peanut oil, cottonseed oil; safflower oil; sesame oil; olive oil: corn oil and soybean oil; glycols; such a propylene glycol or polyethylene glycol: esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The compounds of the invention can be formulated into pharmaceutical compositions for administration to animals or humans. In some embodiments, these pharmaceutical compositions comprise an amount of the PAR-2 signaling pathway inhibitor effective to treat or prevent the diseases or conditions described herein and a pharmaceutically acceptable carrier, adjuvant, or excipient.

The exact amount of compound required for treatment will vary from subject to subject, depending on the species, age, and general condition of the subject, the severity of the infection, the particular agent, its mode of administration, and the like. The compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

In some embodiments, these compositions optionally further comprise one or more additional therapeutic agents. Some embodiments provide a simultaneous, separate or sequential use of a combined preparation.

### Uses and Methods of Treatment

One aspect of this invention provides compounds that are inhibitors of the PAR-2 signaling pathway and composition comprising such compounds, as described above. Another aspect of the invention provides methods and uses for treating or lessening the severity of a disease, condition, or disorder where PAR-2 is implicated in the disease, condition, or disorder, which employ administering a compound of the invention, such as a compound of formula I or a pharmaceutically acceptable salt thereof, or a pharmaceutical composition of the invention comprising such compound or a pharmaceutically acceptable salt thereof. Such methods and uses typically employ administering an effective amount of a compound or pharmaceutical composition of the invention to a patient or subject.

The terms, “disease”. “disorder”, and “condition” may be used interchangeably here to refer to medical or pathological condition which is mediated by PAR-2 or modulated the PAR-2 signaling pathway, or where PAR-2 is implicated in the disease state.

As used herein, the terms “subject” and “patient” are used interchangeably. The terms “subject” and “patient” refer to an animal (e.g., a bird such as a chicken, quail or turkey, or a mammal), specifically a “mammal” including a non-primate (e.g., a cow, pig, horse, sheep, rabbit, guinea pig, rat, cat, dog, and mouse) and a primate (e.g., a monkey, chimpanzee and a human), and more specifically a human. In one embodiment, the subject is a non-human animal such as a farm animal (e.g., a horse, cow, pig or sheep), or a pet (e.g., a dog, cat, guinea pig or rabbit). In a preferred embodiment, the subject is a “human”.

As used herein, an “effective amount” refers to an amount sufficient to elicit the desired biological response. In the present invention, certain examples of the desired biological response is to treat, prevent, or lessen the severity of a disease, condition, or disorder where PAR-2 is implicated in the disease state, to treat a PAR-2 mediated disease, condition, or disorder, to modulate the PAR-2 signaling pathway, to inhibit the PAR-2 signaling pathway, or to enhance or improve the prophylactic or therapeutic effect(s) of another therapy used against a PAR-2 impicated or mediated disease, condition, or disorder, or a disease, condition, or disorder modulated the PAR-2 signaling pathway. The precise amount of compound administered to a subject will depend on the mode of administration, the type and severity of the disease, condition, or disorder and on the characteristics of the patient, such as general health, age, sex, body weight and tolerance to drugs. The skilled artisan will be able to determine appropriate dosages depending on these and other factors. When co-administered with other agents, an “effective amount” of the second agent will depend on the type of drug used. Suitable dosages are known for approved agents and can be adjusted by the skilled artisan according to the condition of the patient, the type of condition(s) being treated and the amount of a compound described herein being used. In cases where no amount is expressly noted, an effective amount should be assumed. For example, compounds described herein can be administered to a subject in a dosage range from between approximately 0.01 to 100 mg/kg body weight/day for therapeutic or prophylactic treatment. The compounds and compositions, according to the method of the present invention, may be administered using any amount and any route of administration effective for eliciting the desired biological response.

As used herein, the terms “treat,” “treatment” and “treating” refer to both therapeutic and prophylactic treatments. For example, therapeutic treatments includes the reduction or amelioration of the progression, severity and/or duration of conditions, diseases or disorders, or the amelioration of one or more symptoms (specifically, one or more discernible symptoms) of conditions, diseases or disorders, resulting from the administration of one or more therapies (e.g., one or more therapeutic agents such as a compound or composition of the invention).

In one embodiment, the invention provides a method of treating a PAR-2 mediated disease, condition, or disorder in a subject in need thereof.

In another embodiment, the invention provides a method for treating or lessening the severity of a disease, condition, or disorder where PAR-2 is implicated in the disease state.

In another embodiment, the invention provides a method for treating or lessening the severity of a disease, condition, or disorder where inhibition of PAR-2 signaling pathway is implicated in the treatment of the disease.

In yet another embodiment, the invention provides a method for treating or lessening the severity of a disease, condition, or disorder by modulating the PAR-2 signaling pathway.

In some embodiments, said disease, condition, or disorder is selected from such diseases, conditions, or disordersin which inhibitors of the PAR-2 signaling pathway may show therapeutic benefit. In some embodiments, said disease, condition, or disorder is selected from inflammatory disease or nociception (pain). In some embodiments, the nociception is caused by inflammation, cancer or injury. In some embodiments, said disease, condition, or disorder is selected from inflammatory bowel disease (e.g., Crohn's disease or ulcerative colitis), irritable bowel syndrome, asthma, rheumatoid arthritis, osteoarthritis, fibrosis (liver fibrosis, pulmonary fibrosis, cystic fibrosis, renal fibrosis, peritoneal fibrosis, pancreatic fibrosis, scleroderma, cardiac fibrosis, skin fibrosis, or intestinal fibrosis), gingivitis, periodontitis, vasculitis (e.g., Wegener's granulomatosis), atopic dermatitis, psoriasis. Netherton syndrome, systemic lupus erythematosus (SLE), scleroderma, interstitial lung disease, polymyositis, dermatomyositis, uveitis, Alzheimer's disease, Parkinson's disease, multiple sclerosis, inflammatory pain, post-operative incision pain, neuropathic pain, fracture pain, osteoporotic fracture pain, and gout joint pain in a patient. Additional diseases that show an increased proteolytic activity may benefit from inhibitors of the PAR-2 signaling pathway.

In some embodiments, said disease, condition, or disorder is selected from diet-induced obesity, adipose inflammation, or metabolic dysfunction. In some embodiments, the metabolic dysfunction correlates with PAR-2 expression.

In some embodiments, said disease, condition, or disorder is selected from cancers. The term “cancer” means a disease characterized by unregulated cell growth. Examples of cancer for which the compounds of the invention can be used include, but not limited to, colorectal cancer, pancreatic cancer, breast cancer, gastric cancer, ovarian cancer, squamous cell carcinoma, uterine endometrial cancer, nasopharyngeal carcinoma, esophageal adenocarcinoma, renal cell carcinoma and glioblastoma. Additional cancers that show an increased proteolytic activity or involvement of tissue factor and the coagulation cascade may benefit from inhibitors of the PAR-2 signaling pathway.

In some embodiments, said disease, condition, or disorder is selected from defects of excessive angiogenesis as manifested in solid tumor growth, tumor metastasis, multiple myeloma, lymphoma, ocular angiogenesis-mediated disorders (diabetic retinopathy, macular degeneration, and other ocular angiogenesis disorders), and angiogenesis-mediated inflammatory disorders.

In some embodiments, said disease, condition, or disorder is fibrosis. Examples of fibrosis includes, but is not limited to, liver fibrosis, pulmonary fibrosis, cystic fibrosis, renal fibrosis, peritoneal fibrosis, pancreatic fibrosis, scleroderma, and cardiac fibrosis.

In some embodiments, the invention provides a method for treating, preventing, or reducing inflammation, nociception (pain) or pruritus in a patient. Yet another embodiment provides a method for treating inflammatory bowel disease (e.g., Crohn's disease or ulcerative colitis), irritable bowel syndrome, asthma, rheumatoid arthritis, osteoarthritis, fibrosis (liver fibrosis, pulmonary fibrosis, cystic fibrosis, renal fibrosis, peritoneal fibrosis, pancreatic fibrosis, scleroderma, cardiac fibrosis, skin fibrosis, or intestinal fibrosis), gingivitis, periodontitis, vasculitis (e.g., Wegener's granulomatosis), atopic dermatitis, psoriasis, Netherton syndrome, systemic lupus erythematosus (SLE), scleroderma, interstitial lung disease, polymyositis, dermatomyositis, uveitis, Alzheimer's disease, Parkinson's disease, multiple sclerosis, inflammatory pain, post-operative incision pain, neuropathic pain, fracture pain, osteoporotic fracture pain, and gout joint pain in a patient. Yet another embodiment provides a method for treating inflammatory bowel disease (e.g., Crohn's disease or ulcerative colitis) and osteoarthritis.

In some embodiments, the invention provides a method for treating or preventing inflammation or pain. Another embodiment provides a method for reducing inflammation.

Another embodiment of this invention provides a method for treating, preventing, or lessening the severity of an inflammatory disease.

Another embodiment of the invention relates to a method of inhibiting the PAR-2 signaling pathway in a patient. In another embodiment, the invention provides a method for inhibiting PAR-2 in a patient.

Another embodiment of the invention provides a method for inhibiting proteolytic activation of PAR-2 in a cell. Another embodiment of the invention provides a to a method of inhibiting PAR-2 signaling pathway activity in a cell.

The invention also provides uses of a compound or composition of the invention for the methods described above. In one embodiment, the invention provides uses in the manufacture of a medicament for such uses, for example, for treating a PAR-2 mediated disease in a patient, for treating, preventing or reducing inflammation or nociception (pain) in a patient, and for treating inflammatory bowel disease (e.g., Crohn's disease or ulcerative colitis), irritable bowel syndrome, asthma, rheumatoid arthritis, osteoarthritis, fibrosis (liver fibrosis, pulmonary fibrosis, cystic fibrosis, renal fibrosis, peritoneal fibrosis, pancreatic fibrosis, scleroderma, cardiac fibrosis, skin fibrosis, or intestinal fibrosis), gingivitis, periodontitis, vasculitis (e.g., Wegener's granulomatosis), atopic dermatitis, psoriasis. Netherton syndrome, systemic lupus erythematosus (SLE), scleroderma, interstitial lung disease, polymyositis, dermatomyositis, uveitis, Alzheimer's disease, Parkinson's disease, multiple sclerosis, inflammatory pain, post-operative incision pain, neuropathic pain, fracture pain, osteoporotic fracture pain, and gout joint pain, fibrosis, cancer, diet-induced obesity, adipose inflammation, and metabolic dysfunction correlating with PAR2 expression in a patient. Yet another embodiment provides uses in the manufacture of a medicament for treating inflammatory bowel disease (e.g., Crohn's disease or ulcerative colitis) and osteoarthritis. Yet another embodiment provides uses in the manufacture of a medicament for use in treating inflammation or pain.

Another aspect provides the use of a compound or composition of the invention in the manufacture of a medicament for use in inhibiting proteolytic activation of PAR-2 in a cell. Another aspect provides a compound or composition of of the invention in the manufacture of a medicament for inhibiting PAR-2 activity in a cell.

Some embodiments comprising co-administering to a patient an additional therapeutic agent, wherein said additional therapeutic agent is appropriate for the disease, condition or disorder being treated; and said additional therapeutic agent is administered together with a compound of the invention as a single dosage form, or separately from said compound as part of a multiple dosage form.

As used herein, the terms “in combination” or “co-administration” can be used interchangeably to refer to the use of more than one therapy (e.g., one or more prophylactic and/or therapeutic agents). The use of the terms does not restrict the order in which therapies (e.g., prophylactic and/or therapeutic agents) are administered to a patient.

Coadministration encompasses administration of the first and second amounts of the compounds of the coadministration in an essentially simultaneous manner, such as in a single pharmaceutical composition, for example, capsule or tablet having a fixed ratio of first and second amounts, or in multiple, separate capsules or tablets for each. In addition, such coadministration also encompasses use of each compound in a sequential manner in either order.

### Modes of Administration and Dosage Forms

The pharmaceutically acceptable compositions of this invention can be administered to humans and other animals orally, rectally, parenterally, intracistemally, intravaginally, intraperitoneally, topically (as by powders, ointments, or drops), bucally, as an oral or nasal spray or via inhalation, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

The compositions of the present invention may be administered orally, parenterally, by inhalation spray, topically, rectally, nasally, buccally, vaginally or via an implanted reservoir. The term “parenteral” as used herein includes, but is not limited to, subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, capsules, tablets, aqueous suspensions or solutions. In the case of tablets for oral use, carriers commonly used include, but are not limited to, lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavouring or colouring agents may also be added.

Alternatively, the pharmaceutical compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include, but are not limited to, cocoa butter, beeswax and polyethylene glycols.

The pharmaceutical compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of inhibitor that may be combined with the carrier materials to produce a single dosage form will vary depending upon the host treated, the particular mode of administration. Preferably, the compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of inhibitor will also depend upon the particular compound in the composition.

Administering with Another Agent

Depending upon the particular conditions to be treated or prevented, additional drugs, which are normally administered to treat or prevent that condition, may be administered together with the compounds of this invention.

Those additional agents may be administered separately, as part of a multiple dosage regimen. Alternatively, those agents may be part of a single dosage form, mixed together with the inhibitor of the PAR-2 signaling pathway in a single composition.

### Biological Samples

As inhibitors of the PAR-2 signaling pathway, the compounds and compositions of this invention are also useful in biological samples. One aspect of the invention relates to inhibiting PAR-2 activity in a biological sample, which method comprises contacting said biological sample with a compound described herein or a composition comprising said compound. The term “biological sample”, as used herein, means an in vitro or an ex vivo sample, including, without limitation, cell cultures or extracts thereof, biopsied material obtained from a mammal or extracts thereof; and blood, saliva, urine, feces, semen, tears, or other body fluids or extracts thereof. The term “compounds described herein” includes compounds of formula I.

Inhibition of PAR-2 signaling pathway activity in a biological sample is useful for a variety of purposes that are known to one of skill in the art. Examples of such purposes include, but are not limited to, blood transfusion, organ-transplantation, and biological specimen storage.

### Study of GPCRs

Another aspect of this invention relates to the study of GPCRs in biological and pathological phenomena; the study of pathways mediated by such GPCRs; and the comparative evaluation of new GPCRs. Examples of such uses include, but are not limited to, biological assays such as enzyme assays and cell-based assays.

The activity of the compounds as inhibitors of the PAR-2 signaling pathway may be assayed in vitro, in vivo or in a cell line. In vitro assays include assays that determine inhibition of either synthetic activators of PAR-2 such as SLIGKV-NH₂ or protease-dependent activators such as trypsin activation of PAR-2.

Another aspect of the invention provides a method for modulating PAR-2 activation by contacting a compound described herein with PAR-2.

### SCHEMES AND EXAMPLES

The compounds of the disclosure may be prepared in light of the specification according to the schemes below as well as according to steps generally known to those of ordinary skill in the art. The compounds may be analyzed by known methods, including but not limited to LCMS (liquid chromatography mass spectrometry) and NMR (nuclear magnetic resonance).

Mass spectrometry samples are analyzed on a Waters UPLC Acquity mass spectrometer operated in single MS mode with electrospray ionization. Samples are introduced into the mass spectrometer using chromatography. Mobile phase for the mass spectrometry analyses consisted of 0.1% formic acid and acetonitrile-water mixture. As used herein, the term “Rt time” refers to the LC-MS retention time, in minutes, associated with the compound. Unless otherwise indicated, the LC-MS methods utilized to obtain the reported retention time are as detailed below:
Method A: 5%-85% acetonitrile-water over 6 minutes run time, Waters AcquityHSS T3 1.8 μm, 2.1 mm ID×50 mm. Flow rate is 1.0 mL/min.
Method B: 50%-100% acetonitrile-water over 6 minutes run time, Waters AcquityHSS T3 1.8 μm, 2.1 mm ID×50 mm. Flow rate is 1.0 mL/min.
Method C: 5%-85% acetonitrile-water over 3 minutes run time, Waters AcquityHSS T3 1.8 μm, 2.1 mm ID×50 mm. Flow rate is 1.0 mL/min.
Method D: 10%-100% acetonitrile-water over 6 minutes run time, Waters XSelect CHS™ C18 2.5 μm, 4.6 mm ID×30 mm Column XP. Flow rate is 1.0 mL/min.
Method E: 5° %-85% acetonitrile-water over 6 minutes run time, Waters Acquity UPLC® HSS C18 SB 1.8 μm, 2.1 mm ID×50 mm. Flow rate is 1.0 mL/min.
Method F: 5-85% acetonitrile-water over 2 minutes run time, Waters AcquityHSS T3 1.8 μm, 2.1 mm ID×50 mm. Flow rate is 1.0 mL/min.
Method G: 50%-90% acetonitrile-water over 6 minutes run time, Waters AcquityHSS T3 1.8 μm, 2.1 mm ID×50 mm. Flow rate is 1.0 mL/min.
Method H: 10%-100% acetonitrile-water over 5 minutes run time, Waters XSelect CHS™ C18 2.5 μm, 4.6 mm ID×30 mm Column XP. Flow rate is 1.0 mL/min.
Method I: 50-100% acetonitrile-water over 2 minutes run time, Waters AcquityHSS T3 1.8 μm, 2.1 mm ID×50 mm. Flow rate is 1.0 mL/min.
Method J: 15%-98% acetonitrile-water over 1.5 minutes run time, Waters AcquityHSS T3 1.8 μm, 2.1 mm ID×30 mm. Flow rate is 1.5 mL/min.
Method K: 0%-50% acetonitrile-water over 6 minutes run time, Waters AcquityHSS T3 1.8 μm, 2.1 mm ID×50 mm. Flow rate is 1.0 mL/min.
Method L: 0%⁰-50% acetonitrile-water over 3 minutes run time, Waters AcquityHSS T3 1.8 μm, 2.1 mm ID×50 mm. Flow rate is 1.0 mL/min.
Method M: 15-98% acetonitrile-10 mM ammonium bicarbonate, pH 10 in water over 1.5 minutes run time, Waters CSHC18, 1.7 um, 2.1 mm ID×30 mm. Flow rate is 1.3 mL/min.

Purification by reverse phase HPLC is carried out under standard conditions using a Phenomenex Gemini 21.2 mm ID×250 mm column, 5μ and Gemini 21.2 mm ID×75 mm column, 5μ, 110 Å. Elution is performed using a linear gradient CH₃CN—H₂O (with or without 0.01% TFA or formic acid buffer) as mobile phase. Solvent system is tailored according to the polarity of the compound, Flow rate, 20 mL/min. Compounds are collected either by UV or Waters 3100 Mass Detector, ESI Positive Mode. Fractions containing the desired compound are combined, concentrated (rotary evaporator) to remove excess CH₃CN and the resulting aqueous solution is lyophilized to afford the desired material.

Nuclear magnetic resonance (NMR) spectra are recorded on INOVA 400 MHz Varian instrument. The residual solvent protons (¹H) are used as internal standards. The following solvents are used: chloroform-d, methanol-d₄, DMSO-d₆. ¹H NMR data are presented as follows: chemical shift in ppm downfield from tetramethylsilane (multiplicity, coupling constant, integration). The following abbreviations are used in reporting NMR data: s, singlet; d, doublet; t, triplet; q, quartet; p, pentuplet; h, hextuplet; dd, doublet of doublets; ddd, doublet of doublets of doublets; dddd, doublet of doublets of doublets of doublets; dt, doublet of triplets; dtd, doublet of triplets of doublets; ddt, doublet of doublets of triplets; dq, doublet of quartets: dp, doublet of pentuplets; td, triplet of doublets; qd, quintet of doublets; m, multiplet.

Purification by flash chromatography on silica gel is carried out under standard conditions using, but not restricted to, either of the following instruments and supplies: Biotage® SP1 or SP2 purification system with Biotage® SNAP Cartridge KP-Sil column 10 g, 25 g, 50 g, 100 g or 340 g or. Biotage® SNAP Cartridge KP-C18-HS 12 g, 30 g, 60 g or 120 g and, CombiFlash®Rf Teledyne Isco purification system with Silica RediSep®Rf normal phase column 12 g, 24 g, 40 g, 80 g, 120 g, 220 g or 330 g. Solvent system is tailored according to the polarity of the compound. Fractions containing the desired compound are combined and concentrated (rotary evaporator) to remove the solvent and to afford the desired material.

### List of Abbreviations

The following abbreviations are used in the examples below:
Ac acetyl
AcOH acetic acid
Ac₂O acetic anhydride
aq aqueous
ATP adenosine triphosphate
BF₃.OEt₂ boron trifluoride diethyl ether
Bn benzyl
Br₂ Bromine
ACN, CH₃CN acetonitrile
CD₃OD methanol-d4
CDCl₃ chloroform-d
Conc. concentrate
Cs₂CO₃ cesium carbonate
CuI copper(I) iodide
CuSO₄ copper(II) sulfate
CV column volume
° C. degree Celcius
DBU 1,8-diazabicyclo[5.4.0]undec-7-ene
DCM methylene chloride or dichloromethane
DIPEA N,N-diisopropylethylamine
DMAP 4-dimethylaminopyridine
DMF dimethylformamide
DMSO dimethylsulfoxide
DMSO-d6 deutered dimethylsulfoxide
dppf diphenylphosphinoferrocene
Equiv equivalent
EtOAc ethyl acetate
g gram(s)
HATU O-(7-azabenzotriazol-1-yl),N,N,N″,N″-tetramethyluroniumhexafluorophosphate
h hour(s)
HCl hydrochloric acid
Hex hexanes
HPLC high pressure liquid chromatography
LCMS liquid chromatography mass spectrometry
LiOH lithium hydroxide
M molar
MHz megahertz
mg milligram(s)
mL milliliter(s)
mM millimolar
MeOH methanol
MeONa sodium methoxide
MgSO₄ magnesium sulfate
min minute(s)
MS mass spectrometer
MTBE methyl tert-butyl ether
μM micromolar
N normal (molar) concentration
NaHCO₃ sodium bicarbonate
Na₂CO₃ sodium carbonate
NaIO₄ sodium periodate
Na₂SO₄ sodium sulfate
Na₂S₂O₃ sodium thiosulfate
NH₄Cl ammonium chloride
¹HNMR proton nuclear magnetic resonance
NMO N-methylmorpholine-N-oxide
OsO₄ osmium tetroxide
ON overnight
Pd₂(dba)₃ Tris(dibenzylideneacetone)dipalladium(0)
Pd/C palladium on carbon
PdCl₂ palladium (II)chloride
Pd(OAc)₂ palladium(II) acetate
PdCl₂(dppf). DCM (1,1′-Bis-(diphenylphosphino)-ferrocene)palladium (II) dichloride
Pd(OH)₂ dihydroxy palladium
Pd(PPh₃)₄ tetrakis(triphenylphospine)palladium (0)
psi pound per square inch
Py pyridine
r.b.f. (rbf) round bottom flask
RT (rt or r. t.) room temperature
S-Phos 2-Dicyclohexylphosphino-2′,6′-dimethoxybiphenyl
T3P Propylphosphonic anhydride
TBAF tetrabutylammonium fluoride
TBDMSOTf tert-butyldimethylsilyl trifluoromethanesulfonate
TBS tert-butyldimethylsilyl
TEA triethylamine
Tf trifluoromethanesulfonyl
TFA trifluoroacetic acid
THF tetrahydrofuran
TLC thin layer chromatography
TMS trimethylsilyl
TMSI trimethylsilyl iodide
TMSN₃ trimethylsilyl azide
TMSOTf trimethylsilyl trifluoromethanesulfonate
TPAP Tetrapropylammonium perruthenate
UPLC ultra performance liquid chromatography
   The following generic schemes and examples illustrate how to prepare the compounds of the present disclosure

### General Synthetic Route 1

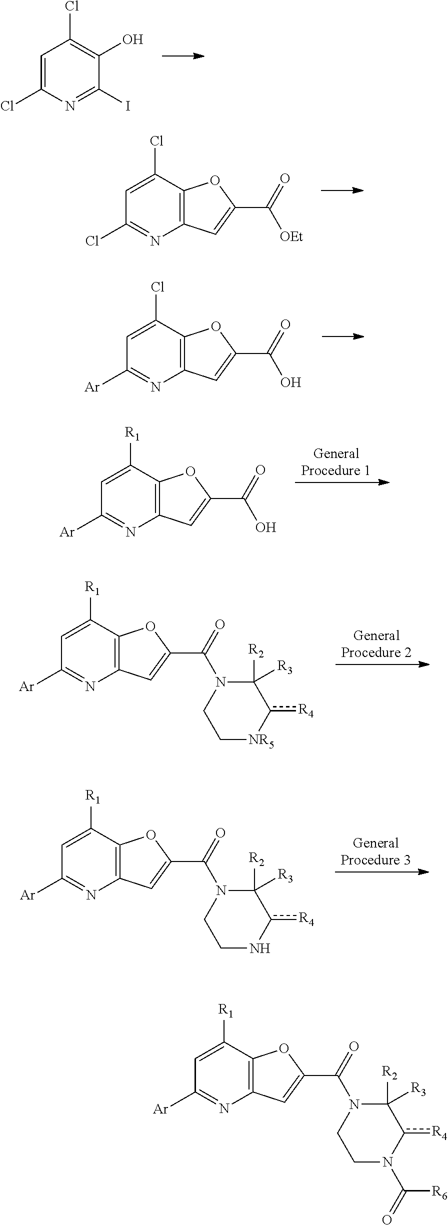

### General Synthetic Route 2

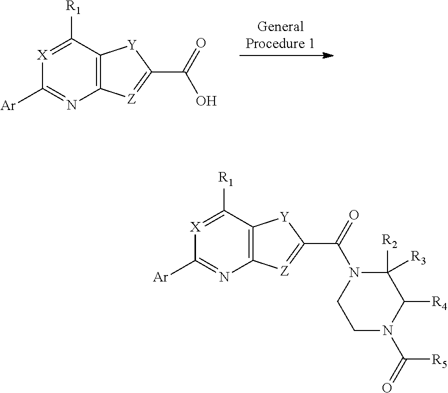

### General Synthetic Route

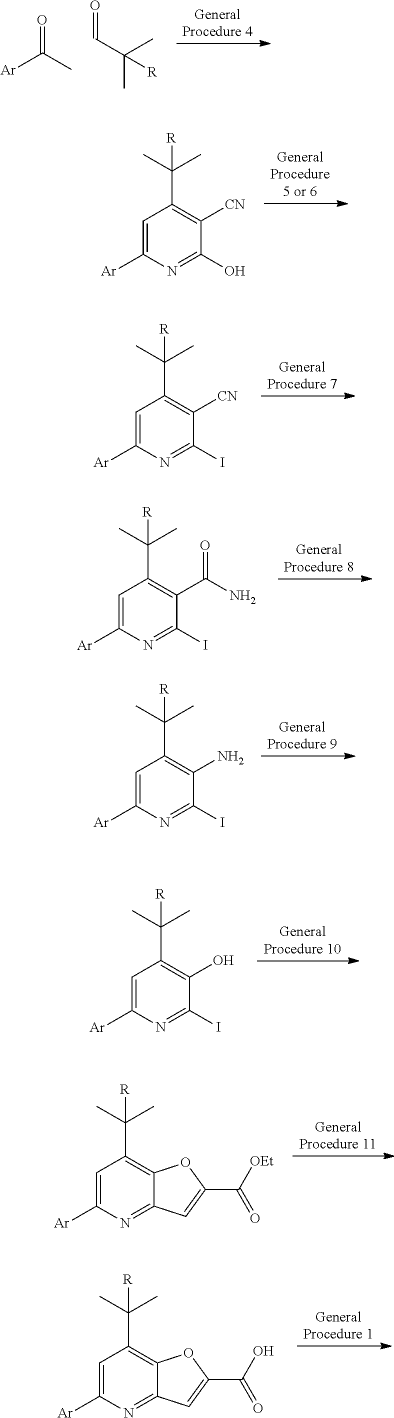

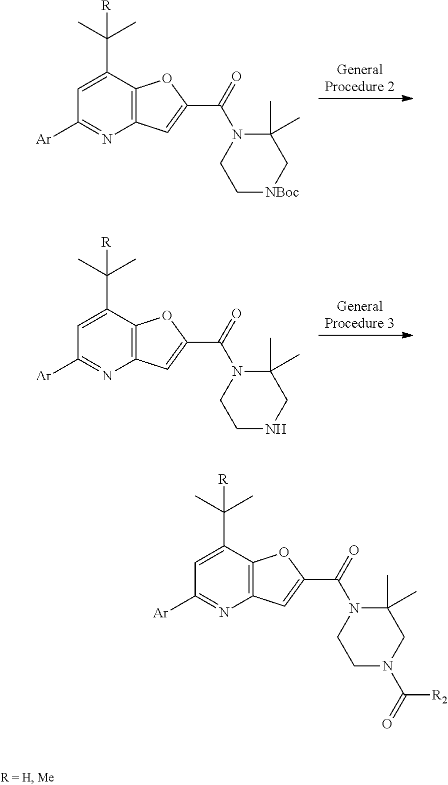

### General Synthetic Route 4

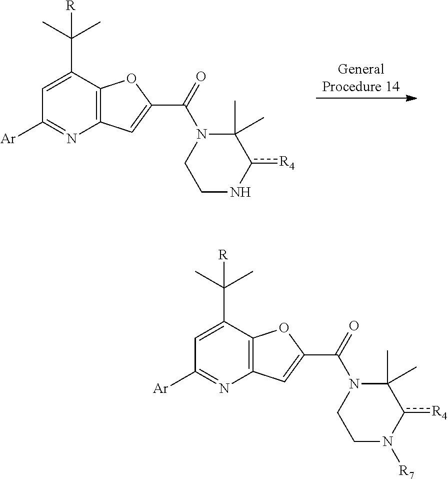

### General Synthetic Route 5

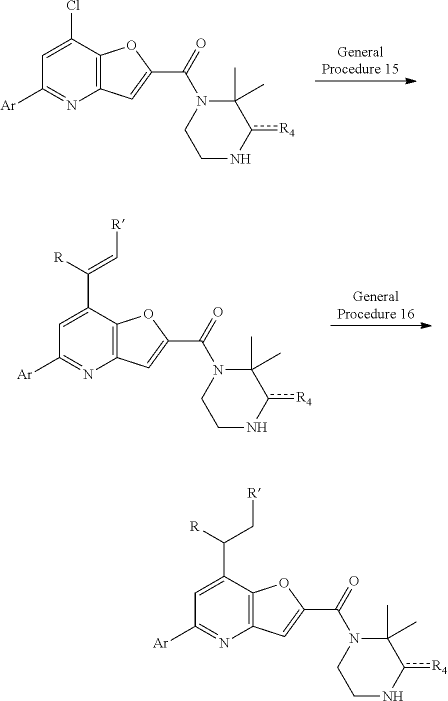

### General Synthetic Route 6

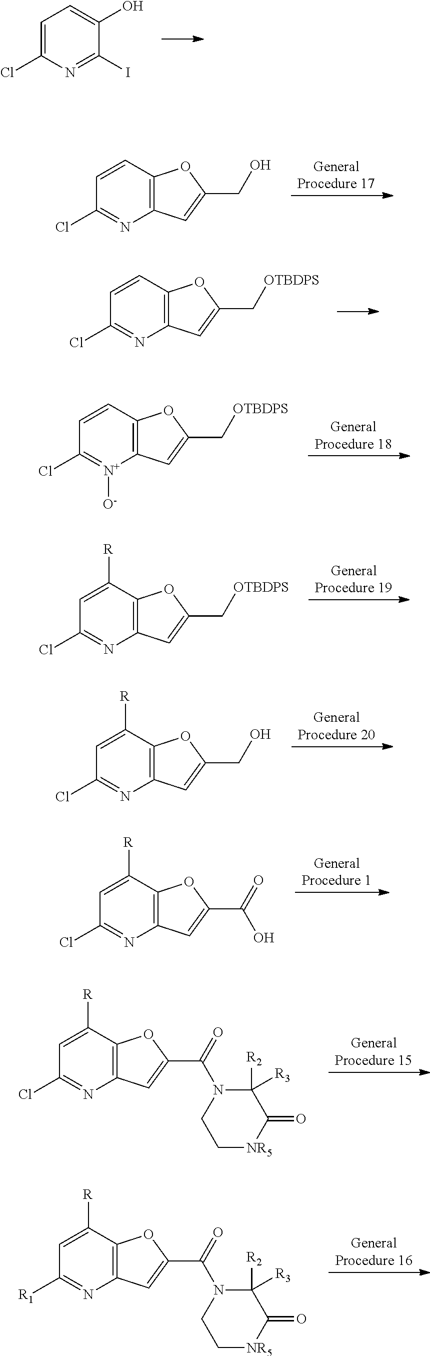

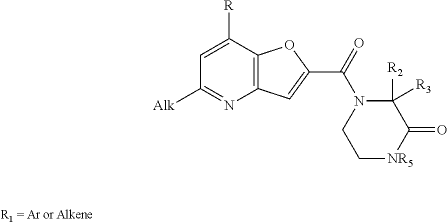

### General Synthetic Route 7

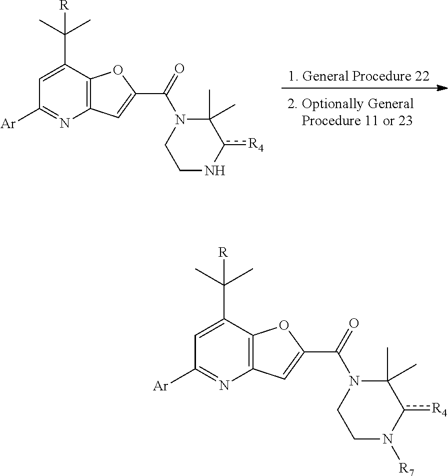

### General Synthetic Route 8

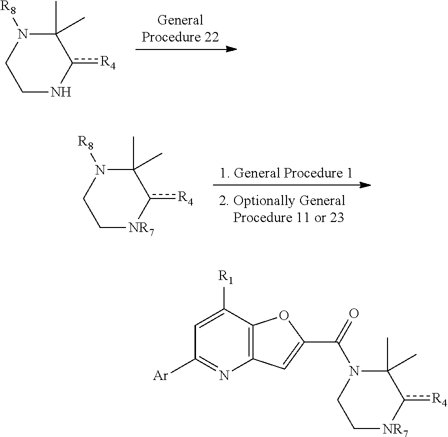

### General Procedure 1: Amide Formation

The appropriate carboxylic acid (1.0 equiv) is dissolved in DMF or NMP (0.03 to 0.4M) before HATU (1.1 to 1.5 equiv), or T3P (1 to 5 equiv) the corresponding amine (1.0 to 1.5 equiv) and Hünig's base (3.0 to 5.0 equiv) are added (the addition order of the reagents may vary) The mixture is stirred at room temperature for 45 min. to 72 h. Either one of these 3 work-up procedures can be employed:

1. Water or aq. ammonium chloride is added and the aq. phase is extracted with EtOAc. The combined organic phase is washed with brine, dried over MgSO₄, filtered and the filtrate evaporated under reduced pressure affording the title compound which is used in the subsequent step without further purification.

2. Water or aq. ammonium chloride is added and the aq. phase is extracted with EtOAc. The combined organic phase is washed with 1N HCl, sat. aq. NaHCO₃ and brine, dried over Na₂SO₄, filtered and the filtrate evaporated under reduced pressure affording the title compound which is used in the subsequent step without further purification.

3. The volatiles are removed under reduced pressure and the residue is purified by flash chromatography on silica gel or by mass-directed reverse phase HPLC, affording the title compound.

### General Procedure 2: Tert-Butyl Carbamate Deprotection

The appropriate boc-protected amine (1.0 equiv) is dissolved in an appropriate solvent such as DCM, 1,4-dioxane or MeOH (or combinations thereof) (0.1 M) before an acid such as 4N HCl solution in 1,4-dioxane or TFA (20.0 to 30.0 equiv) are added. The solution is stirred at room temperature for 1 to 4 h before the volatiles are removed under reduced pressure, affording the title compound which is used in the subsequent step without further purification. In some cases, MeOH is added to solubilize the salts and the resulting solution is allowed to stir for an additional 1 h prior to concentration under reduced pressure.

### General Procedure 3: Amide Formation

A solution of the appropriate piperazine (1.0 equiv) and HATU (1.3 equiv) in NMP or DMF (400 μL to 800 μL) is added to either the corresponding NMP solution of carboxylic acid (200 μL, 0.1 mM), or the corresponding neat acid (1.0 to 1.1 equiv). Hünig's base (3.0 to 5.0 equiv) is added and the solution stirred at room temperature for 20 minutes to 16 hours. Either one of these 3 work-up procedures can be employed:

1. Aq. ammonium chloride is added and the aq. phase is extracted with EtOAc. The combined organic phase is washed with brine, dried over MgSO₄, filtered and the filtrate evaporated under reduced pressure affording the title compound which is used in the subsequent step without further purification.

2. Aq. ammonium chloride is added and the aq. phase is extracted with EtOAc. The combined organic phase is washed with 1N HCl, sat. aq. NaHCO₃ and brine, dried over Na₂SO₄, filtered and the filtrate evaporated under reduced pressure affording the title compound which is used in the subsequent step without further purification.

3. The volatiles are removed under reduced pressure and the residue is purified by flash chromatography on silica gel or by mass-directed reverse phase HPLC, affording the title compound.

### General Procedure 4: Pyridine Synthesis

To a solution of the appropriate acetophenone (1.05 to 1.3 equiv), aldehyde (1.0 equiv) and ethyl 2-cyanoacetate (1.0 equiv) in ethanol (1.0 M) is added ammonium acetate (7.0 to 10 equiv) and the solution is stirred at 80° C. for 6 to 18 h. The solution is cooled to room temperature before water is added and the resulting precipitate recovered by filtration and dried under reduced pressure. If only partial aromatization is observed, the solid is dissolved in DCM (0.25 to 0.9 M) and DDQ (0.5 to 1.0 equiv) is added. The mixture is stirred at room temperature for 4 to 96 h, filtered and the filtrate evaporated under reduced pressure. The residue obtained is triturated from hot ethanol and water, or methanol at room temperature affording the title product as a solid.

### General Procedure 5: Iodination

To a stirring suspension of the appropriate 2-pyridone (1 equiv) and pyridine (1.1 to 1.3 equiv) in acetonitrile (0.5 to 1 M) at 0° C. is added dropwise triflic anhydride (1.05 to 1.25 equiv) and the resulting solution is stirred at room temperature for 30 minutes. NaI (2 to 10 equiv) and HCl 12N (1.1 to 5 equiv) or triflic acid (1 to 1.25 equiv) are added and the solution stirred at 90° C. for 16 h. Water and aq. NaHCO₃ is added and the aq. phase is extracted with EtOAc or DCM. The combined organic phase is washed with brine, dried over MgSO₄, filtered, and the filtrate is evaporated under reduced pressure. The residue is purified either by flash chromatography on silica gel, or by trituration affording the title compound as a solid.

### General Procedure 6: Alternative Iodination Conditions

To a stirring suspension of the appropriate 2-pyridone (1 equiv) and pyridine (1.1 to 1.3 equiv) in acetonitrile (0.5 to 1 M) at 0° C. is added dropwise triflic anhydride (1.05 to 1.25 equiv) and the resulting solution is stirred at room temperature for 30 minutes. The volatiles are removed under reduced pressure and the residue dissolved in NMP (0.5 to 1M) before NaI (2 to 10 equiv) and HCl 12N (1.5 to 5 equiv), or triflic acid (1.5 to 5 equiv) are added and the solution stirred at 80° C., or 90° C. for 16 h. Water and aq. NaHCO₃ are added, and the aq. phase is extracted with EtOAc. The combined organic phase is washed with brine, dried over MgSO₄, filtered and the filtrate evaporated under reduced pressure. The residue is purified either flash chromatography on silica gel or trituration affording the title compound as a solid.

### General Procedure 7: Nitrile Hydrolysis

To a round bottom flask containing the appropriate 2-iodo-pyridine-3-carbonitrile (1 equiv) is added sulfuric acid (10 equiv) and the mixture is stirred at 80° C. to 90° C. for 4 to 72 h. The solution is cooled to room temperature and added dropwise to a saturated solution of sodium carbonate or potassium carbonate in water (10 to 20 equiv). The resulting white precipitate is collected by filtration, washed with water and dried under reduced pressure, affording the product as a white solid.

### General Procedure 8: Hofmann Rearrangement

To a solution of KOH (7 equiv) in water (2.6 volume equiv) at 0° C. is added bromine (1.3 equiv) dropwise and the solution stirred for 5 minutes before the appropriate 2-iodo-pyridine-3-carboxamide (1 equiv) is added portion wise. The suspension is stirred at 0° C. for 5 minutes before careful addition of water (0.9 volume equiv) and THF (0.9 volume equiv) and stirring is pursued for 15 minutes. The solution is stirred at room temperature for 10 minutes to 2 hours. Water is added and the aq. phase is extracted with EtOAc. The combined organic phase is washed with sat. aqueous Na₂SO₃ and brine, and dried over MgSO₄ and filtered. The filtrate is evaporated under reduced pressure affording the title compound which is used in the subsequent step without further purification or purified by flash chromatography on silica gel.

### General Procedure 9: Sandmeyer Reaction

To a solution of the appropriate 3-aminopyridine (1.0 equiv) in TFA (4.7 volume equiv) is added dropwise isopentyl nitrite (1.5 to 6.0 equiv) and the solution stirred for 30 minutes. The solution is diluted with methanol (10 to 20 volume equiv) and K₂CO₃ is added until pH 12 is obtained. The solution is stirred for 1 to 16 h before water is added, the pH is adjusted to 5 using 12N HCl and the aq. phase is extracted with EtOAc or DCM. The combined organic phase is washed with brine, dried over MgSO₄ and filtered. The filtrate is evaporated under reduced pressure affording the title product compound which is used in the subsequent step without further purification or purified by flash chromatography on silica gel.

### General Procedure 10: Sonogashira Cross-Coupling

To a round bottom flask containing a solution of the appropriate 2-iodo-3-hydroxy-pyridine (1.0 equiv) in THF (5.1 volume equiv) is added potassium carbonate (7.0 equiv), PdCl₂(PPh₃)₂ (0.04 equiv) and CuI (0.08 equiv) and the solution is warmed to 80° C. A solution of ethyl prop-2-ynoate (2.5 to 4.0 equiv) in THF (2.6 volume equiv) is added dropwise over 30 minutes to 1 hour. The solution is cooled to room temperature and acidified using 12N HCl. Water is added and the aq. phase is extracted with EtOAc. The combined organic phase is washed with brine, dried over MgSO₄, filtered and the filtrate evaporated under reduced pressure. The residue is purified by flash chromatography on silica gel affording the title compound as a solid.

### General Procedure 11: Ester Hydrolysis

To a solution of the appropriate ester (1.0 equiv) (1.0 equiv) in solvents selected from ethanol, methanol, dioxane, THF, NMP, DMF and DMSO, or combinations thereof (0.15 to 0.3 M) is added a 2M aqueous solution of LiOH (2.0-5.0 equiv), NaOH (2.0-5.0 equiv), or KOH (2.0-5.0 equiv), and the solution is stirred for 1 h to 3 days at temperature ranging from r.t. to 80° C. Either one of these 4 work-up procedures can be employed:

1. Water is added and the solution acidified to pH 2-4 using an appropriate acid such as 1 to 12N HCl or aqueous citric acid. The precipitate is collected by filtration, washed with water and dried under reduced pressure, affording the title compound as a solid. Alternatively the compound can be dissolved in DMSO and purified by mass-directed reverse-phase preparative hplc affording the desired compound.

2. Water is added, the solution acidified to to pH 2-4 using an appropriate acid such as 1 to 12N HCl or aqueous citric acid and the aq. phase is extracted with EtOAc. The combined organic phase is washed with brine, dried over MgSO₄, filtered and the filtrate evaporated under reduced pressure affording the title compound as a solid. Alternatively the compound can be dissolved in DMSO or NMP and purified by mass-directed reverse-phase preparative hplc affording the desired compound.

3. Water is added and the solution acidified using 12N HCl. The precipitate is collected by filtration, washed with water and dried under reduced pressure affording the title compound as a solid. Alternatively the compound can be dissolved in DMSO and purified by mass-directed reverse-phase preparative hplc affording the desired compound.

4. The reaction mixture is filtered and purified by mass-directed reverse-phase preparative hplc affording the desired compound.

### General Procedure 12: Sulfonamide Formation

To a solution of the appropriate amine (1.0 equiv) in DMF (0.05-0.25M) is added triethylamine (2.5 to 5.0 equiv) followed by the appropriate sulfonyl chloride (1.2 to 2.4 equiv). The resulting solution is stirred at a temperature ranging from room temperature to 50° C. for 4 to 16 hours. Upon reaction completion, the crude mixture is purified directly by mass-directed reverse-phase HPLC affording the desired compound.

### General Procedure 13: Ester Hydrolysis

To a solution of the appropriate piperazine amide (1.0 equiv) in dioxane (0.15 to 0.3 M) is added a 2M aqueous solution of LiOH or NaOH (2.0-4.0 equiv) and the solution is stirred for 1-22 h at rt or reflux. Either one of these 2 work-up procedures can be employed: 1. Water is added, the solution acidified to pH 4 using 12 N HCl and the aq. phase is extracted with EtOAc. The combined organic phase is washed with brine, dried over MgSO₄, filtered and the filtrate evaporated under reduced pressure affording the title compound as a solid.

2. The solution is acidified to pH 4 using 4N HCl in dioxane and evaporated in vacuo. The residue is dissolved in DMSO and purified by mass-directed reverse-phase preparative hplc affording the desired compound.

### General Procedure 14: Alkylation or Arylation of Amides

To a solution of the appropriate piperazinone (1.0 equiv) in NMP (0.1-0.25M) is added 60% sodium hydride (1.2 equiv). The resulting solution is stirred at a room temperature for 15-30 minutes before the appropriate haloalkyl or haloaryl/haloheteroaryl (1.0 to 5.0 equiv) is added. The mixture is stirred at a temperature from RT to 150° C. for 1 to 16 hours. Upon completion of the reaction, the crude mixture is purified by mass-directed reverse-phase affording the desired compound.

### General Procedure 15: Suzuki Cross-Coupling

To a rbf containing a solution of the appropriate aryl halide (1 equiv) in dioxane (0.03 to 0.4M) is added Pd₂dba₃.CHCl₃ (0.02 to 0.25 equiv) and S-Phos (0.05 to 0.5, 2 equiv of Pd atom). The resulting solution is degassed with nitrogen for 5 minutes prior to addition of respectively K₃PO₄ (2 to 5 equiv. of a 2M aq. solution) and associated boronic acid or boronate ester (1 to 2.5 equiv). It is then stirred between 60 and 105° C. for 2 to 18 h. DCM or EtOAc and NaHCO₃ are added to the mixture, the layers are separated and the aqueous phase is extracted with DCM or EtOAc twice. The combined organic layers are dried with MgSO₄, filtered and concentrated under reduced pressure to afford a crude product which is used in the subsequent step without further purification or purified by filtration over silica gel or mass-directed reverse-phase HPLC.

### General Procedure 16: Hydrogenation

To a flask containing palladium on carbon or platinum oxide (0.05 to 0.2 equiv) and the appropriate olefin (1 equiv) in EtOH or MeOH (0.03 to 0.1 M) or MeOH:DCM (under a nitrogen atmosphere is evacuated under reduced pressure and re-filled with hydrogen 3 times and the suspension is stirred under hydrogen pressure at room temperature overnight. The suspension is filtered over celite, washed with DCM and the volatiles are removed under reduced pressure. The residue is purified by mass-directed reverse phase HPLC to afford upon lyophilization the desired compound.

### General Procedure 17: Silyl Ether Protection

To a solution of the appropriate primary alcohol (1 equiv) and trialkyl chlorosilane (1.5 equiv) in DMF (0.6 M) is added imidazole (2.5 equiv) at rt and the solution is stirred at rt for 18 h.

Aq. sat. NaHCO: is added and the aq. phase is extracted with EtOAc. The combined organic phase is washed with 1N HCl, sat. aq. NaHCO₃ and brine, dried over Na₂SO₄, filtered and the filtrate evaporated under reduced pressure affording the title compound which is used in the subsequent step without further purification.

Aq. and EtOAc are added to the reaction, the organic phase is extracted with EtOAc to afford the title compound used as is for the next step without further purification.

### General Procedure 18: C-7 Selective Addition

To a round bottom flask containing a solution of tert-butyl-[(5-chloro-4-oxido-furo[3,2-b]pyridin-4-ium-2-yl)methoxy]-diphenyl-silane (1 equiv) and iodocopper (0.4 equiv) in THF (0.5 M) at −5° C. is added trifluoro-tetrahydrofuran-1-ium-1-yl-boron (2.2 equiv) and the solution is allowed to stir for 30 minutes. The mixture is kept at −5° C. and to this is slowly added tert-butyl(chloro)magnesium (2.4 equiv) over 60 minutes. Aq. ammonium chloride is added and the aq. phase is extracted with DCM. The phases are seperated, DDQ (0.11 equiv) is directly added to the organic phase and the mixture is allowed to stir for 20 minutes at room temperature. DDQ (0.10 equiv) is added again and the mixture is allowed to stir for 4 h at it. Water is added, the phases are seperated and the volatiles are removed under reduced pressure affording the title compound which is used in the subsequent step without further purification

### General Procedure 19: Silyl Ether Deprotection

To a round bottom flask containing a solution of tert-butyl-[(7-tert-butyl-5-chloro-furo[3,2-b]pyridin-2-yl)methoxy]-diphenyl-silane (1 equiv) in THF (0.4 M) at rt is added TBAF (2 equiv) and the solution is allowed to stir for 3 h. Water along with DCM is added and the phases are seperated. The solvents are evaporated under reduced pressure and the residue purified by flash chromatography on silica gel affording the title compound as a solid.

### General Procedure 20: Ley Oxidation

To a solution of the appropriate primary alcohol (1 equiv) and NMO monohydrate (10 equiv) in acetonitrile (0.15 to 0.2 M) in a water bath is added TPAP (0.05 equiv to 0.1 equiv) portionwise and the solution is stirred at room temperature for 1 h. The solution is cooled to 0° C. i-PrOH (10 equiv) is added slowly and the reaction is stirred for 15 to 30 minutes. The reaction mixture is diluted with water and the volatiles are removed under reduced pressure. The aq. phase is basified to pH 13 using 2N NaOH and washed with MTBE and the organic phase discarded. The aq. phase is acidified to pH 3 with 2N HCl, the product formed is collected by filtration, washed with water and heptane and dried under reduced pressure affording the title compound as a solid.

### General Procedure 21: TFA Deprotection of PMB Protected Amine

A solution of the appropriate para-methoxybenzyl protected amine (1 equiv) in trifluoroacetic acid (0.06 to 0.2 M) is heated in the microwave or thermally at temperatures ranging from RT to 130° C. for the appropriate time to complete the reaction. The trifluoroacetic acid is removed under reduced pressure. The residue is purified by mass-directed reverse phase HPLC to afford upon lyophilization the desired compound.

### General Procedure 22: C—N Aryl Bond Formation

Method A:

The appropriate piperazine (1.0 equiv) is dissolved in DMF, dioxane, DMSO or NMP (0.01 to 0.4M), treated with a base selected from Cs₂CO₃, DBU and Hünig's base (2.0 to 5.0 equiv) and the appropriate aryl halide (1.0 to 5.0 equiv). The mixture is stirred at a temperature ranging from r.t. to 150° C. for 30 min to 24 h, in the microwave of thermally.

Method B:

The appropriate piperazine or piperazinone (1.0 equiv) is dissolved in DMF, dioxane, DMSO or NMP (0.01 to 0.4M), treated with a base selected from NaOtBu, K₃PO₄ and K₂CO₃ (2.0 to 5.0 equiv), the appropriate aryl halide (1.0 to 5.0 equiv) and the solution is flushed with nitrogen prior to adding a palladium catalyst selected from palladium acetate, RuPhos(IV), and a ligand selected from RuPhos, JohnPhos, XPhos and XantPhos. The mixture is stirred at a temperature ranging from r.t. to 150° C. for 30 min to 24 h, in the microwave of thermally.

Method C:

The appropriate piperazinone (1.0 equiv) is dissolved in toluene, DMF, dioxane or NMP (0.01 to 0.4M), treated with a base selected from Cs₂CO₃, K₃PO₄ and K₂CO₃ (2.0 to 5.0 equiv), added the appropriate aryl halide (1.0 to 5.0 equiv) and CuI (1.0 equiv to 3 equiv), optionally with ligands such as N,N′-dimethylethylenediamine or N,N,N′N′-tetramethylethylenediamine (0.1 equiv to 3 equiv). The mixture is stirred at a temperature ranging from r.t. to 150° C. for 30 min to 24 h, in the microwave of thermally.

Method D:

Under nitrogen, sodium hydride 60% dispersed in mineral oil (1.15 eq) is added to a stirred mixture of the appropriate piperazinone (1.0 eq) in DMF. The mixture is stirred for 15 min-1 hour at rt and then, the appropriate aryl halide (1.5 eq) is added. The reaction mixture is stirred for 2-16 hours at 60° C. thermally.

Either one of these 3 work-up procedures can be employed:

1. Water is added, and the aq. phase is extracted with EtOAc. The combined organic phase is washed with brine, dried over MgSO₄, filtered and the filtrate evaporated under reduced pressure affording the title compound as a solid.

2. The volatiles are removed under reduced pressure and the residue is purified by flash chromatography on silica gel or by mass-directed reverse phase HPLC, affording the title compound.

3. Water is added (a solid crashed out) and the resulting precipitate is allowed to stir for 1 h. The heterogenous mixture is filtered on Buchner and the desired solid is washed with water, followed by heptane to give the desired product.

In the case where the product is substituted with an ester functional group, the ester group may be hydrolyzed by either of General Procedure 11 or General Procedure 23.

### General Procedure 23: Acidic Ester Hydrolysis

A solution of the appropriate tert-butylester (1.0 equiv) in solvents selected from methanol, dioxane, THF, NMP, DCM or DMF (or combinations thereof) (0.15 to 0.3 M) is treated with an appropriate acid such as HCl in dioxane (1.0 equiv to 10 equiv) or TFA and stirred at a temperature ranging from r.t. to 80° C. for 30 min to 24 h.

### General Procedure 24: Preparation of Tetrazoles

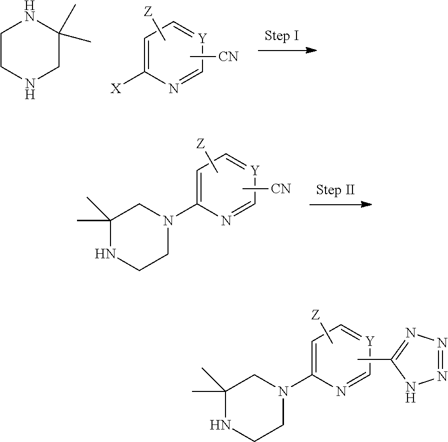

### Step I

Reaction is carried out using the appropriate heteroaryl nitrile and 2,2-dimethylpiperazine using General Procedure 22A, to afford the desired product.

### Step II

A mixture of the appropriate nitrile (1 eq.), sodium azide (3 eq) and triethylamine hydrochloride (3 eq.) in DMF (0.1 to 0.5M) is heated in the microwave for 2 to 3 hours at 130° C. The cooled mixture is evaporated to dryness and dried under vacuum. The residue is purified by reverse phase chromatography, using 0 to 30% water in ACN gradient to afford the desired tetrazole.

### List of Intermediates

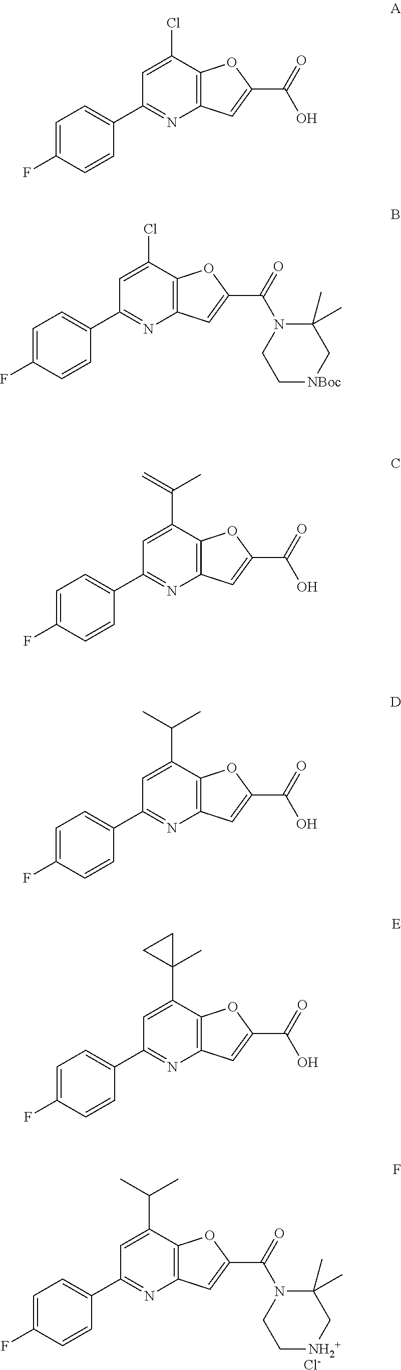

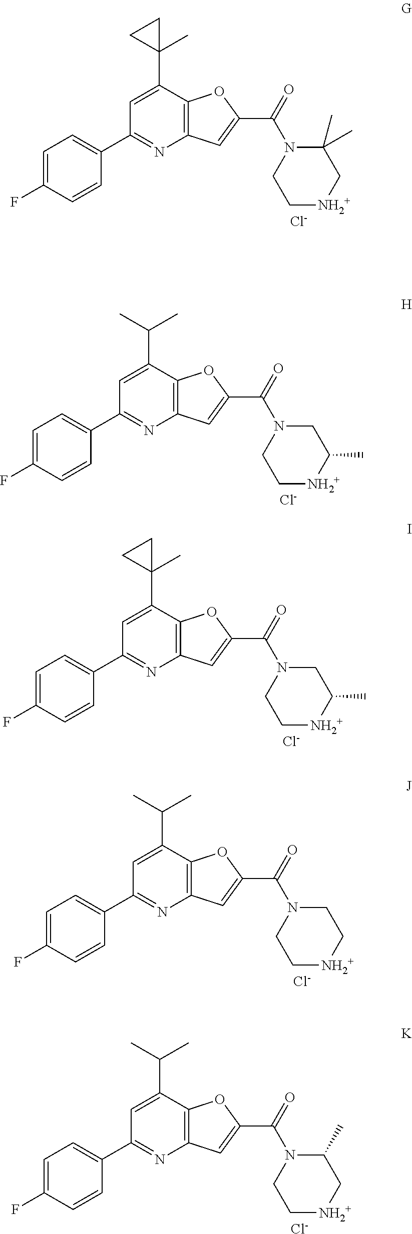

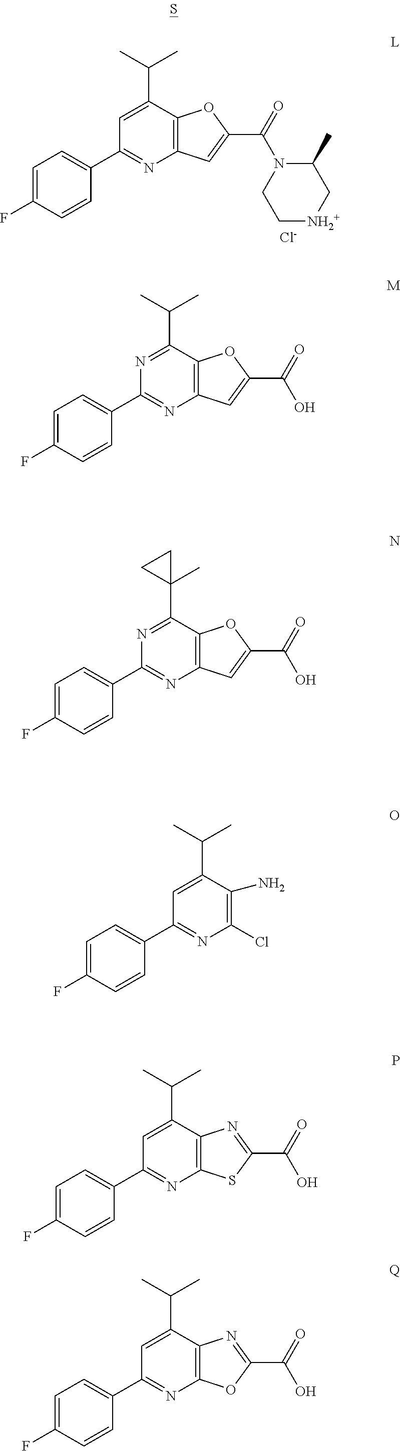

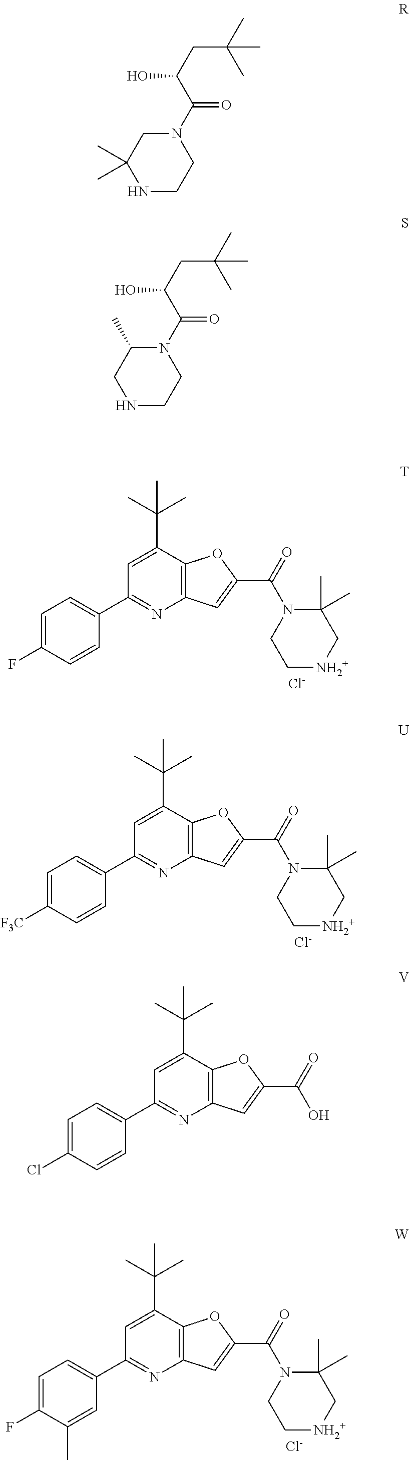

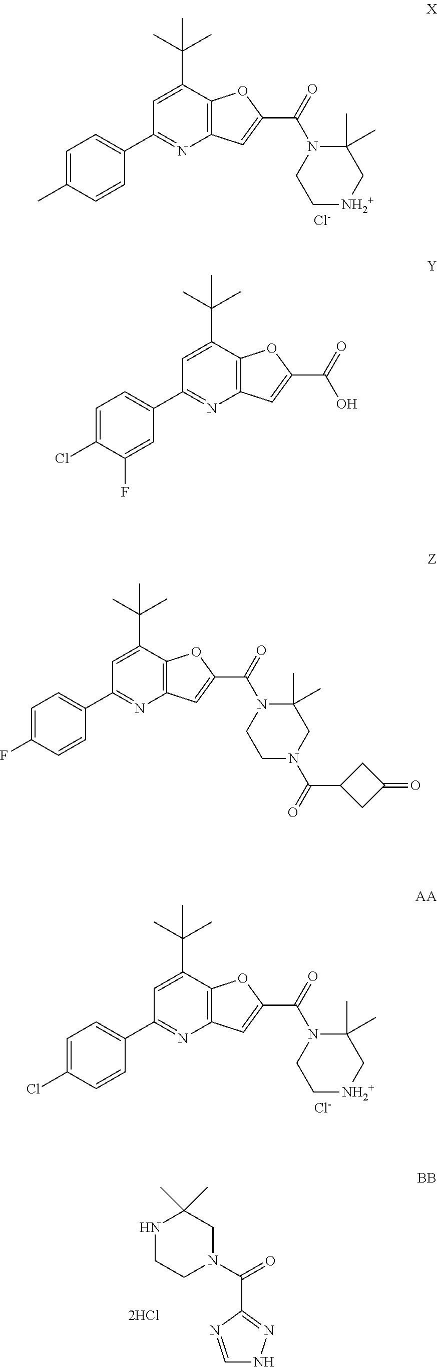

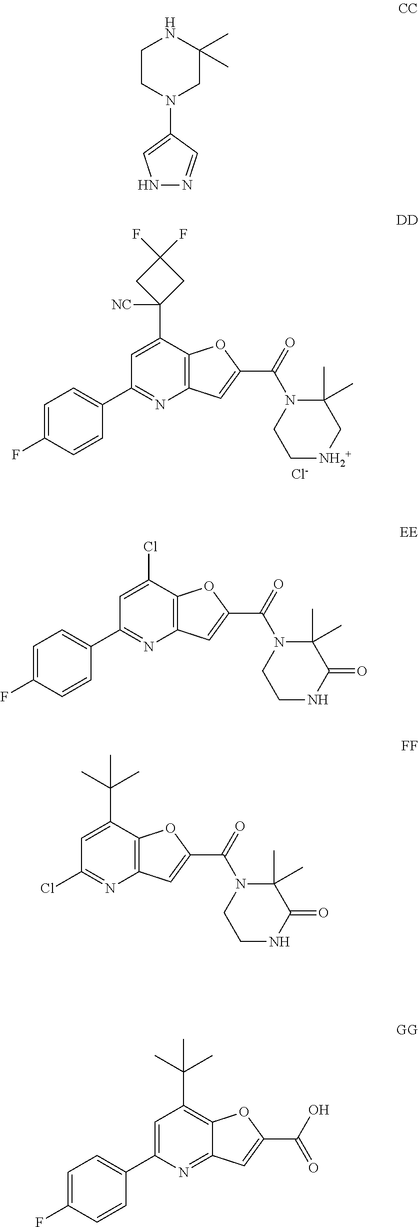 [SMILES: CC1(C)CN(c2cn[nH]c2)CCN1 ; CC1(C)C[NH2+]CCN1C(=O)c1cc2nc(-c3ccc(F)cc3)cc(C3(C#N)CC(F)(F)C3)c2o1 ; [Cl-] ; CC1(C)C(=O)NCCN1C(=O)c1cc2nc(-c3ccc(F)cc3)cc(Cl)c2o1 ; CC(C)(C)c1cc(Cl)nc2cc(C(=O)N3CCNC(=O)C3(C)C)oc12 ; CC(C)(C)c1cc(-c2ccc(F)cc2)nc2cc(C(=O)O)oc12]

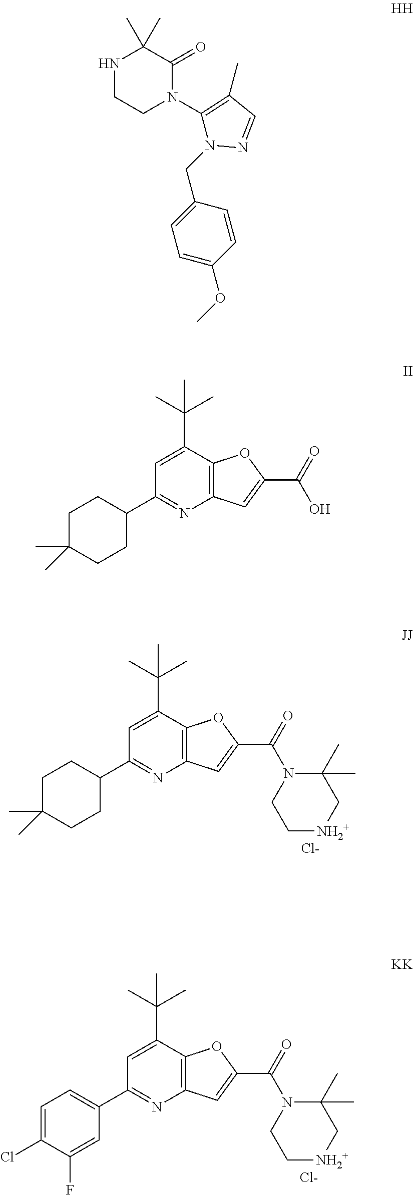

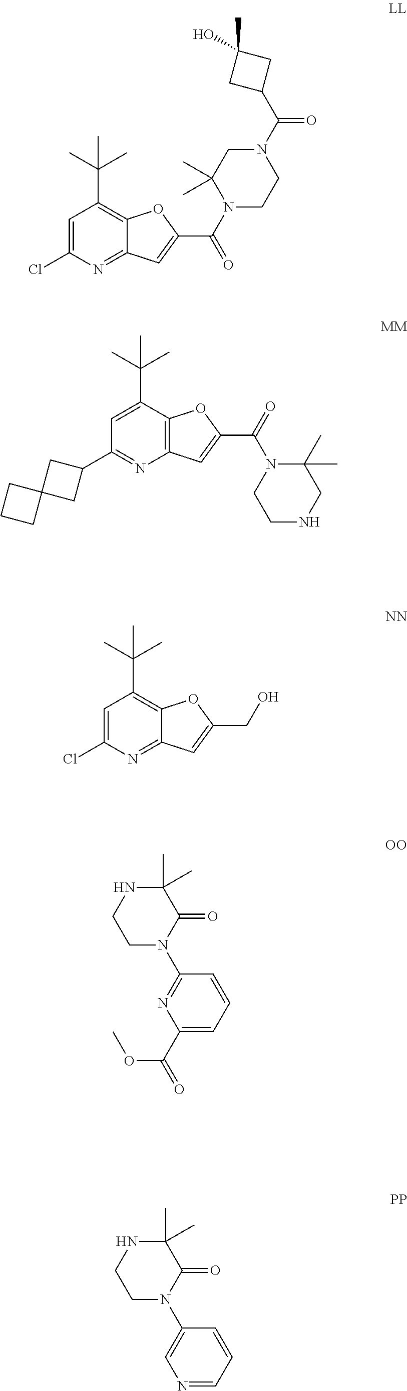

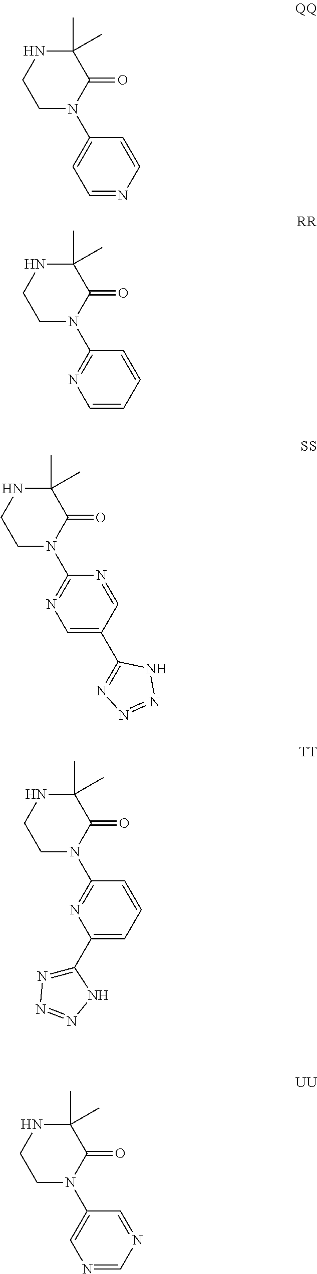

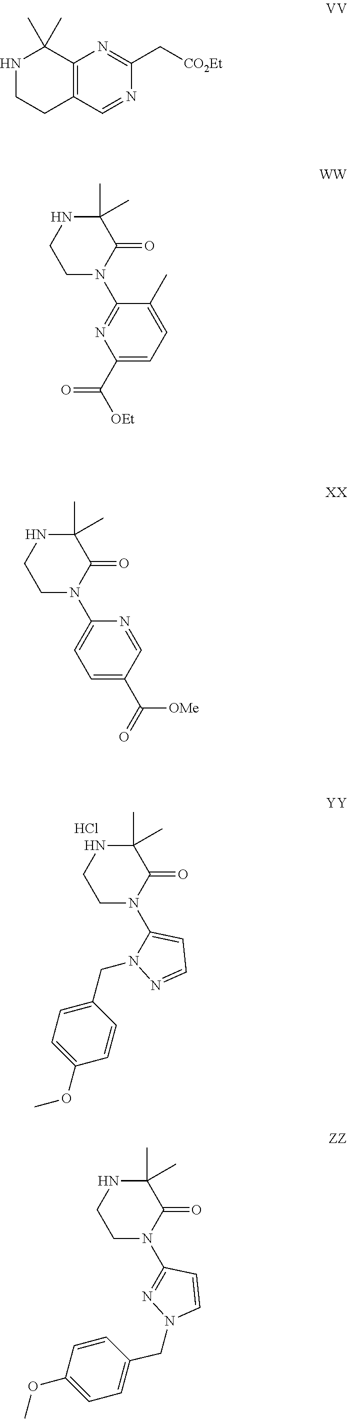

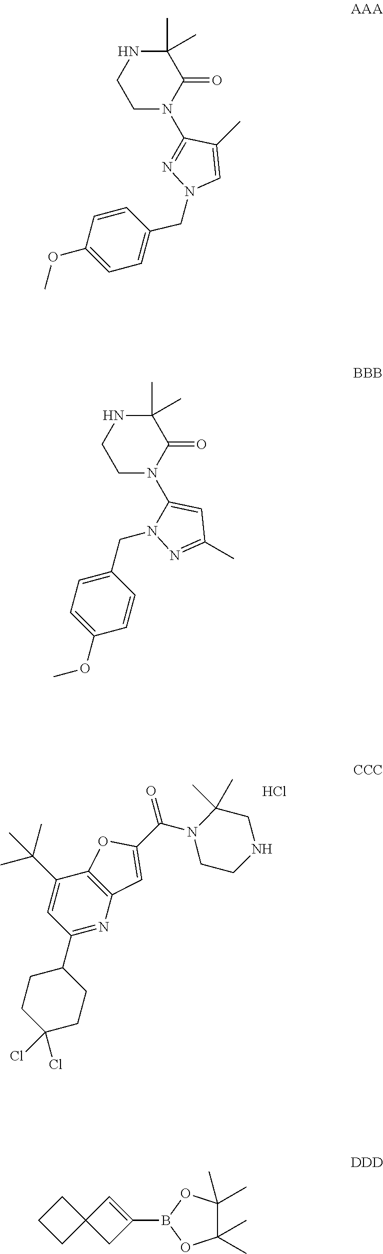

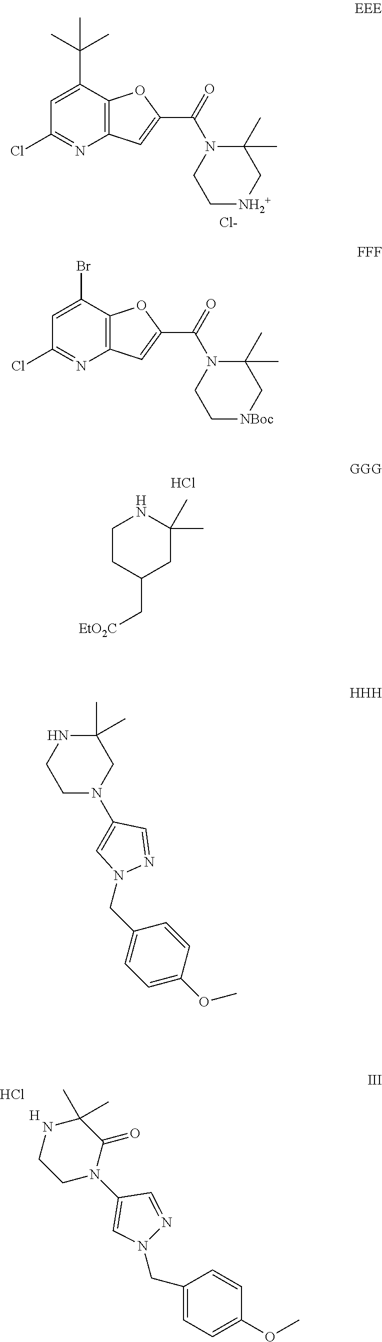

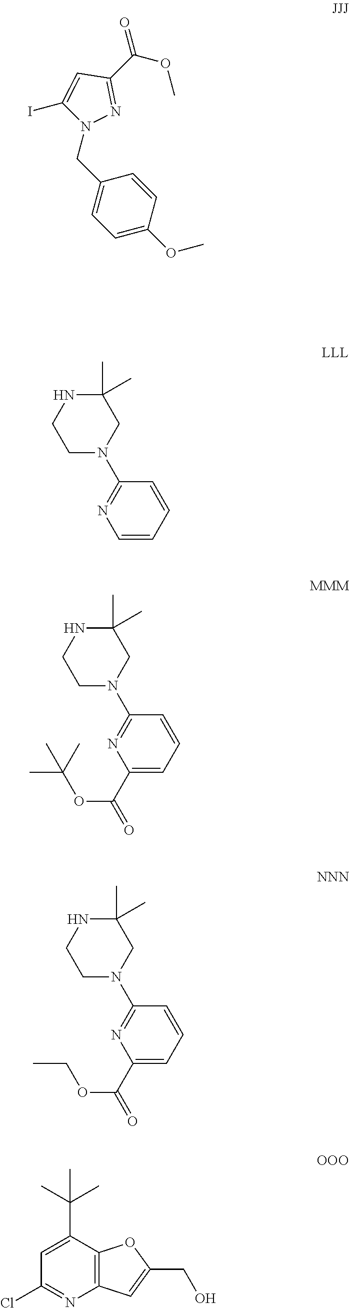

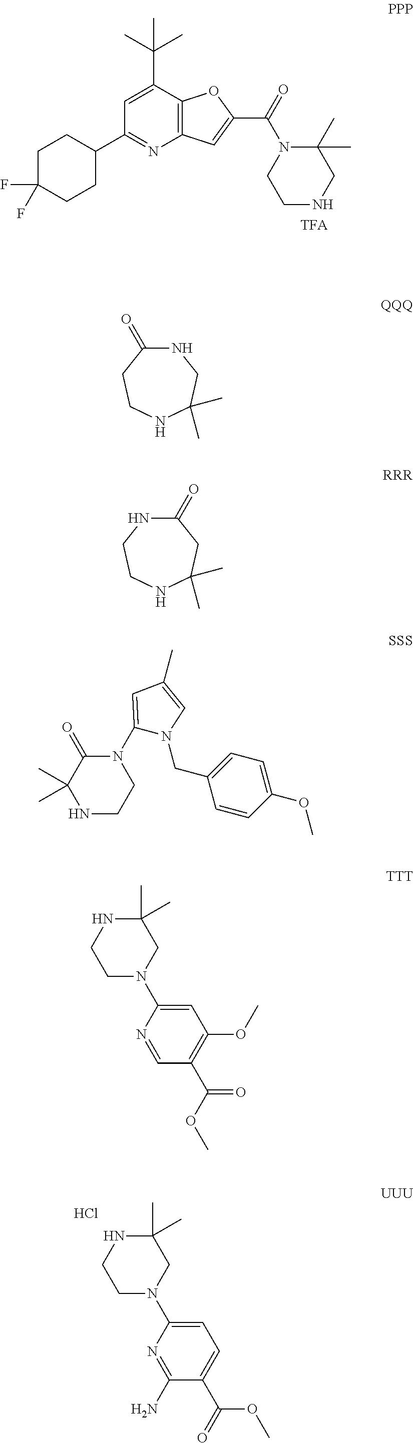

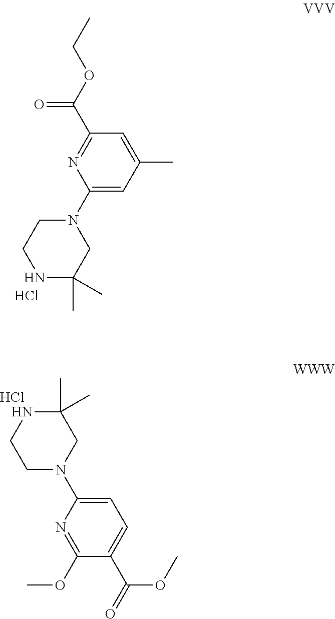 [SMILES: Cl ; CCOC(=O)c1cc(C)cc(N2CCNC(C)(C)C2)n1 ; Cl ; COC(=O)c1ccc(N2CCNC(C)(C)C2)nc1OC]

### Preparation of Intermediate A: 7-Chloro-5-(4-fluorophenyl)furo[3,2-b]pyridine-2-carboxylic acid

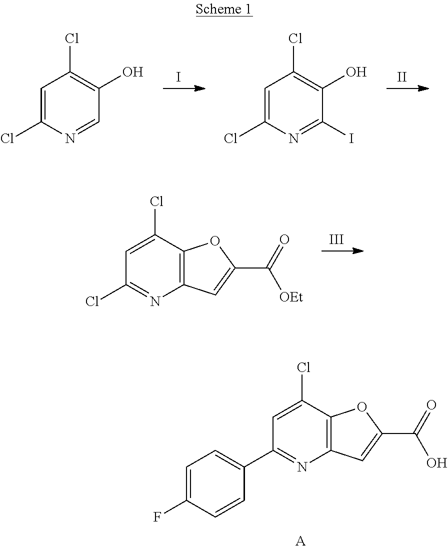

### Step I: 4,6-Dichloro-2-iodo-pyridin-3-ol

To a solution of 4,6-dichloropyridin-3-ol (5.00 g, 30.5 mmol) and K₂CO₃ (8.43 g, 61.0 mmol) in water (80 mL) was added iodine (8.43 g, 1.71 mL, 33.2 mmol) and the solution was stirred for 3 h. To the brown solution was added sodium sulfite portionwise until the dark brown color vanished and the solution was acidified to pH 1 using 12N HCl. The white precipitate was collected by filtration and washed with water, affording the title compound. 4,6-Dichloro-2-iodo-pyridin-3-ol (7.81 g, 880/yield). ¹H NMR (400 MHz, Chloroform-d) δ 7.28 (s, 1H), 5.80 (s, 1H). ESI-MS m/z calc. 288.8558, found 290.06 (M+H)⁺; Retention time: 1.9 minutes using method C.

### Step II: Ethyl 5,7-dichlorofuro[3,2-b]pyridine-2-carboxylate

To a round bottom flask containing a solution of 4,6-dichloro-2-iodo-pyridin-3-ol (6.38 g, 22.01 mmol) in THF (33 mL) was added potassium carbonate (21.30 g, 154.1 mmol), PdCl₂(PPh₃)₂ (618 mg, 0.88 mmol) and CuI (335 mg, 1.76 mmol) and the solution was warmed to 80° C. A solution of ethyl prop-2-ynoate (8.92 mL, 88.0 mmol) in THF (16 mL) was added dropwise over one hour and stirring is pursued for 2 h Another portion of ethyl prop-2-ynoate (2.23 mL, 22.0 mmol) in THF (5 mL) was added dropwise over 30 minutes and the solution stirred at 80° C. for further 2 h. The solution was cooled to room temperature and acidified using 12N HCl. Water was added and the aq. phase was extracted with EtOAc. The combined organic phase was washed with brine, dried over MgSO₄, filtered and the filtrate evaporated under reduced pressure. The residue was purified by flash chromatography on silica gel eluting with 0-40% EtOAc: Hexanes affording the title compound. Ethyl 5,7-dichlorofuro[3,2-b]pyridine-2-carboxylate (2.80 g. 49% yield). ¹H NMR (400 MHz, DMSO-d6) δ 8.02-7.93 (m, 2H), 4.39 (q, J=7.1 Hz. 2H), 1.32 (t, J=7.1 Hz, 3 Hz). ESI-MS m/z calc. 258.9803, found 260.11 (M+1)⁺; Retention time: 1.57 minutes using method C.

### Step III: 7-Chloro-5-(4-fluorophenyl)furo[3,2-b]pyridine-2-carboxylic acid (Intermediate A)

To a solution of ethyl 5,7-dichlorofuro[3,2-b]pyridine-2-carboxylate (3.50 g, 13.46 mmol) in 1,4-dioxane (135 mL) was added an aqueous solution of LiOH (10.10 mL of 2 M, 20.19 mmol) and the solution stirred at room temperature for 1 h before Na₂CO₃ (20.19 mL of 2 M, 40.38 mmol), Pd(PPh₃)₄ (778 mg, 0.67 mmol) and 4-fluorophenylboronic acid (1.81 g, 12.92 mmol) are added. Nitrogen was bubbled in the reaction mixture for 5 minutes before it was heated to 60° C. for 18 h. Water (300 mL) was added, the solution was acidified using 12N HCl and the precipitate formed collected by filtration. The solid was washed with water and dried under reduced pressure, affording the title compound which was used in the subsequent step without further purification. 7-Chloro-5-(4-fluorophenyl)furo[3,2-b]pyridine-2-carboxylic acid (3.93 g, 100% yield). ESI-MS m/z calc. 291.00986, found 292.17 (M+1)⁺; Retention time: 1.36 minutes using method C.

### Preparation of Intermediate B: tert-Butyl 4-[7-chloro-5-(4-fluorophenyl)furo[3,2-b]pyridine-2-carbonyl]-3,3-dimethyl-piperazine-1-carboxylate

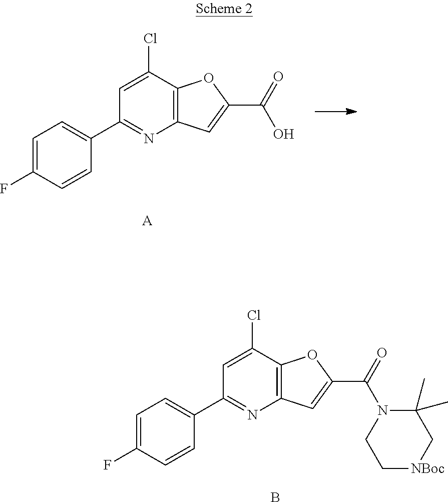

7-Chloro-5-(4-fluorophenyl)furo[3,2-b]pyridine-2-carboxylic acid (Intermediate A) (390 mg, 1.34 mmol) was dissolved in DMF (6 mL) before HATU (661 mg, 1.74 mmol), tert-butyl 3,3-dimethylpiperazine-1-carboxylate (287 mg, 1.34 mmol) and Hünig's base (932 μL, 5.35 mmol) were added. The solution was stirred at room temperature for 3 h, and then saturated aqueous NH₄Cl is added. The aq. phase was extracted twice with EtOAc. The combined organic phase was washed with 1N HCl, saturated aqueous NaHCO₃ and brine. The organic phase was then dried over MgSO₄, filtered and the filtrate evaporated under reduced pressure. The crude product was used in the subsequent step without further purification. tert-Butyl 4-[7-chloro-5-(4-fluorophenyl)furo[3,2-b]pyridine-2-carbonyl]-3,3-dimethyl-piperazine-1-carboxylate (170 mg, 24% yield). ESI-MS m/z calc. 487.16742, found 488.39 (M+1)⁺; Retention time: 2.16 minutes using method C.

### Preparation of Intermediate C: 5-(4-Fluorophenyl)-7-isopropenyl-furo[3,2-b]pyridine-2-carboxylic acid

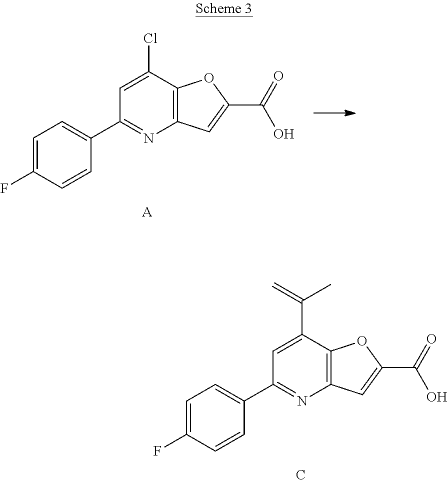

To a round bottom flask containing a solution of 7-chloro-5-(4-fluorophenyl)furo[3,2-b]pyridine-2-carboxylic acid (Intermediate A) (7.50 g, 25.71 mmol) in dioxane (159 mL) was added Pd₂dba₃ (235 mg, 0.26 mmol) and S-Phos (401 mg, 0.98 mmol) and the solution was degassed with nitrogen for 5 minutes before K₃PO₄ (51.40 mL of 2 M, 102.8 mmol) and 2-isopropenyl-4,4,5,5-tetramethyl-1,3,2-dioxaborolane (8.64 g, 51.42 mmol) were added and the solution stirred at 90° C. for 16 h. The solution was cooled to room temperature and water was added (300 mL). The dark solid formed was removed by filtration and the filtrate acidified with 12N HCl. The precipitate formed was collected by filtration, was washed with water and dried under reduced pressure, affording the title compound. 5-(4-Fluorophenyl)-7-isopropenyl-furo[3,2-b]pyridine-2-carboxylic acid (4.60 g, 60% yield). ESI-MS m/z calc. 297.0801, found 298.23 (M+1)⁺; Retention time: 1.45 minutes using method C.

### Preparation of Intermediate D: 5-(4-Fluorophenyl)-7-isopropyl-furo[3,2-b]pyridine-2-carboxylic acid

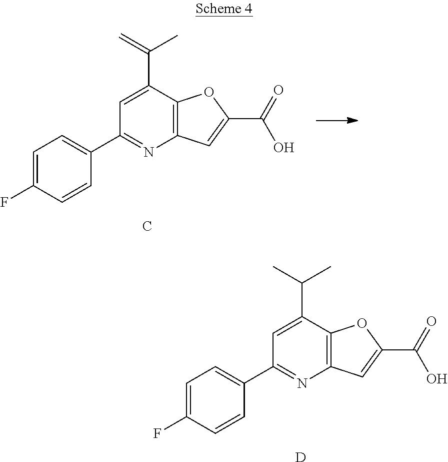

To a Parr flask containing 10% w/w palladium on carbon (372 mg, 0.35 mmol) under a nitrogen atmosphere was added of solution of 5-(4-fluorophenyl)-7-isopropenyl-furo[3,2-b]pyridine-2-carboxylic acid (Intermediate C) (2.08 g, 7.00 mmol) in EtOH (87 mL). The flask was placed on the Parr shaker and the air is removed by vacuum and replaced with nitrogen twice. The flask was then evacuated under reduced pressure and re-filled with hydrogen 3 times and the suspension was shaken under a 40 PSI pressure of hydrogen for 48 h. Hydrogen was evacuated and the flask backfilled with nitrogen before the catalyst is filtered on Celite® and washed with DCM. The filtrate was evaporated under reduced pressure affording the title compound. 5-(4-Fluorophenyl)-7-isopropyl-furo[3,2-b]pyridine-2-carboxylic acid (1.83 g, 87% yield). ESI-MS m/z calc. 299.09576, found 300.24 (M+1)⁺; Retention time: 2.8 minutes using method A.

### Preparation of Intermediate E: 5-(4-Fluorophenyl)-7-(l-methylcyclopropyl)furo[3,2-b]pyridine-2-carboxylic acid

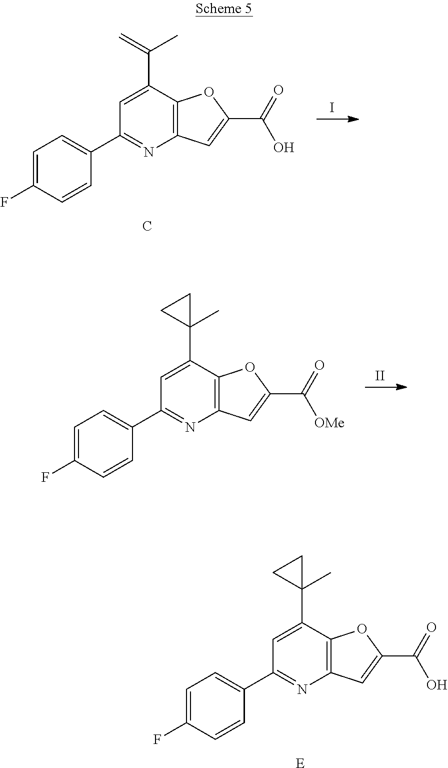

### Step I: Methyl 5-(4-fluorophenyl)-7-(1-methylcyclopropyl)furo[3,2-b]pyridine-2-carboxylate

To a solution of 5-(4-fluorophenyl)-7-isopropenyl-furo[3,2-b]pyridine-2-carboxylic acid (Intermediate C) (2.46 g, 8.28 mmol) in DCM (62 mL) was added a solution of diazomethane (25.00 mL of 1M in diethyl ether, 25.00 mmol) to form the methyl ester. Palladium acetate (186 mg, 0.83 mmol) was added and the solution cooled to 0° C. before dropwise addition of a solution of diazomethane (496.5 mL of 1 M in diethyl ether, 496.5 mmol) over 1 h. The solution was filtered over silica and the filtrate evaporated under reduced pressure. The product was re-dissolved in DCM (62 mL) and palladium acetate (186 mg, 0.83 mmol) was added. The solution was cooled to 0° C. before diazomethane (496.5 mL of 1 M in diethyl ether, 496.5 mmol) was added dropwise over 1 h. The solution was filtered over silica and the filtrate evaporated under reduced pressure affording the desired product. Methyl 5-(4-fluorophenyl)-7-(1-methylcyclopropyl)furo[3,2-b]pyridine-2-carboxylate (2.70 g, 100/yield). ESI-MS m/z calc. 325.11142, found 326.3 (M+1)⁺; Retention time: 3.93 minutes using method A.

### Step II: 5-(4-Fluorophenyl)-7-(1-methylcyclopropyl)furo[3,2-b]pyridine-2-carboxylic acid (Intermediate E)

To a solution of methyl 5-(4-fluorophenyl)-7-(1-methylcyclopropyl)furo[3,2-b]pyridine-2-carboxylate (2.70 g, 8.30 mmol) in dioxane (42 mL) was added an aqueous solution of LiOH (8.30 mL of 2 M, 16.60 mmol) and the solution was stirred at room temperature for 16 h. Water was added and the solution acidified using 12N HCl. The solid formed was collected by filtration, washed with water and dried under reduced pressure, affording the title compound (2.01 g). The filtrate was extracted with EtOAc and the combined organic phases were washed with brine, dried over MgSO₄, filtered and the filtrate was evaporated under reduced pressure affording the title compound (460 mg). The two crops were combined, affording the title compound. 5-(4-Fluorophenyl)-7-(1-methylcyclopropyl)furo[3,2-b]pyridine-2-carboxylic acid (2.47 g, 96% yield). ESI-MS m/z calc. 311.09576, found 312.61 (M+1)⁺; Retention time: 2.73 minutes using method A.

### Preparation of Intermediate F: (2,2-Dimethylpiperazin-1-yl)-[5-(4-fluorophenyl)-7-isopropyl-furo[3,2-b]pyridin-2-yl]methanone hydrochloride

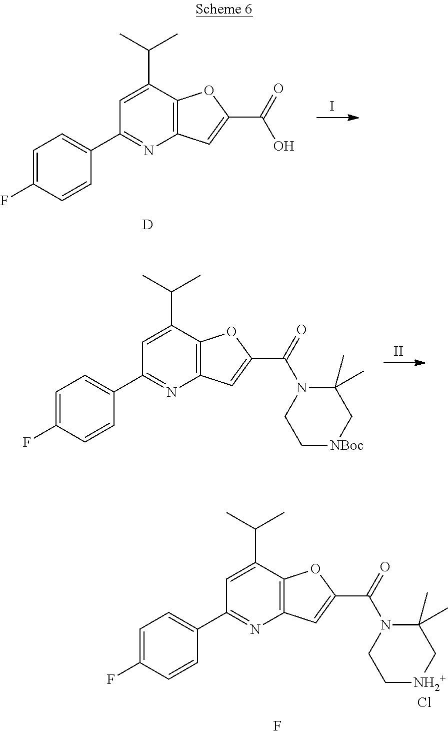

### Step I: tert-Butyl 4-[5-(4-fluorophenyl)-7-isopropyl-furo[3,2-b]pyridine-2-carbonyl]-3,3-dimethyl-piperazine-1-carboxylate

The product was prepared according to General Procedure 1 using Intermediate D (750 mg, 2.51 mmol), HATU (1.24 g, 3.26 mmol), DMF (12 mL), Hünig's base (1.31 mL, 7.52 mmol) and tert-butyl 3,3-dimethylpiperazine-1-carboxylate (591 mg, 2.76 mmol) affording the title compound. tert-Butyl 4-[5-(4-fluorophenyl)-7-isopropyl-furo[3,2-b]pyridine-2-carbonyl]-3,3-dimethyl-piperazine-1-carboxylate (1.24 g, 100% yield). An aliquot of the crude product was purified by mass-directed reverse phase HPLC affording the title compound, the remaining product was used in the subsequent step without further purification. ¹H NMR (400 MHz, DMSO-d6) δ 8.23-8.14 (m, 2H), 7.88 (s, 1H), 7.50 (s, 1H), 7.33 (t, J=8.9 Hz, 2H), 3.86 (s, 2H), 3.58-3.39 (m, 5H), 1.49 (s, 6H), 1.42 (d, J=7.7 Hz, 15H). ESI-MS m/z calc. 495.25333, found 496.79 (M+1)⁺; Retention time: 2.02 minutes using method C.

### Step II: (2,2-Dimethylpiperazin-1-yl)-[5-(4-fluorophenyl)-7-isopropyl-furo[3,2-b]pyridin-2-yl]methanone hydrochloride (Intermediate F)

The product was prepared according to General Procedure 2 using tert-butyl 4-[5-(4-fluorophenyl)-7-isopropyl-furo[3,2-b]pyridine-2-carbonyl]-3,3-dimethyl-piperazine-1-carboxylate (1.24 g, 2.51 mmol), DCM (25 mL) and HCl in dioxane (6.27 mL of 4 M, 25.06 mmol) affording the title compound. (2,2-Dimethylpiperazin-1-yl)-[5-(4-fluorophenyl)-7-isopropyl-furo[3,2-b]pyridin-2-yl]methanone hydrochloride (1.08 g, 100% yield). ESI-MS m/z calc. 395.20091, found 396.36 (M+1)⁺; Retention time: 1.09 minutes using method C.

### Preparation of Intermediate G: (2,2-Dimethylpiperazin-1-yl)-[5-(4-fluorophenyl)-7-(1-methylcyclopropyl)furo[3,2-b]pyridin-2-yl]methanone hydrochloride

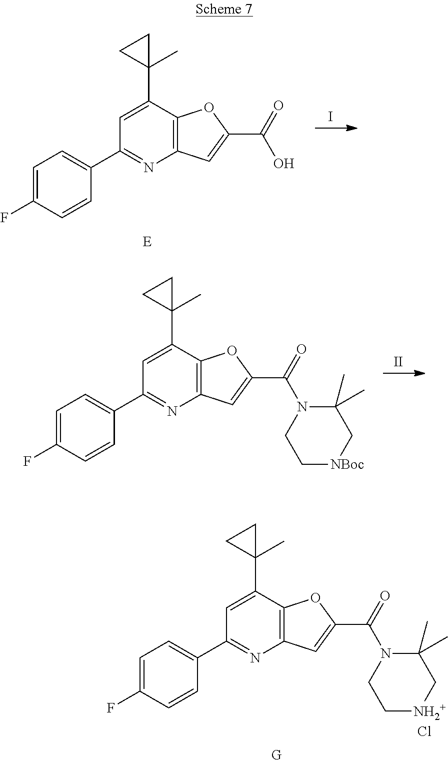

### Step I: tert-Butyl 4-[5-(4-fluorophenyl)-7-(1-methylcyclopropyl)furo[3,2-b]pyridine-2-carbonyl]-3,3-dimethyl-piperazine-1-carboxylate

The product was prepared according to General Procedure 1 using Intermediate E (1.50 g, 4.83 mmol), HATU (2.39 g, 6.28 mmol), DMF (25 mL), Hünig's base (2.95 mL, 16.92 mmol) and tert-butyl 3,3-dimethylpiperazine-1-carboxylate (1.19 g. 5.56 mmol) affording the title compound. 4-[5-(4-Fluorophenyl)-7-(1-methylcyclopropyl)furo[3,2-b]pyridine-2-carbonyl]-3,3-dimethyl-piperazine-1-carboxylate (2.50 g, 100% yield). ESI-MS m/z calc. 507.25333, found 508.73 (M+1)⁺; Retention time: 2.04 minutes using method C.

### Step II: (2,2-Dimethylpiperazin-1-yl)-[5-(4-fluorophenyl)-7-(1-methylcyclopropyl)furo[3,2-b]pyridin-2-yl]methanone hydrochloride (Intermediate G)

The product was prepared according to General Procedure 2 using 4-[5-(4-fluorophenyl)-7-(l 1-methylcyclopropyl)furo[3,2-b]pyridine-2-carbonyl]-3,3-dimethyl-piperazine-1-carboxylate (2.50 g, 4.92 mmol), DCM (53 mL) and HCl in dioxane (24.6 mL of 4 M, 98.5 mmol) affording the title compound. (2,2-Dimethylpiperazin-1-yl)-[5-(4-fluorophenyl)-7-(1-methylcyclopropyl)furo[3,2-b]pyridin-2-yl]methanone hydrochloride (2.20 g, 100% yield). ESI-MS m/z calc. 407.2009, found 408.36 (M+1)⁺; Retention time: 1.23 minutes using method C.

### Preparation of Intermediate H: [5-(4-Fluorophenyl)-7-isopropyl-furo[3,2-b]pyridin-2-yl]-[(3S)-3-methylpiperazin-1-yl]methanone hydrochloride

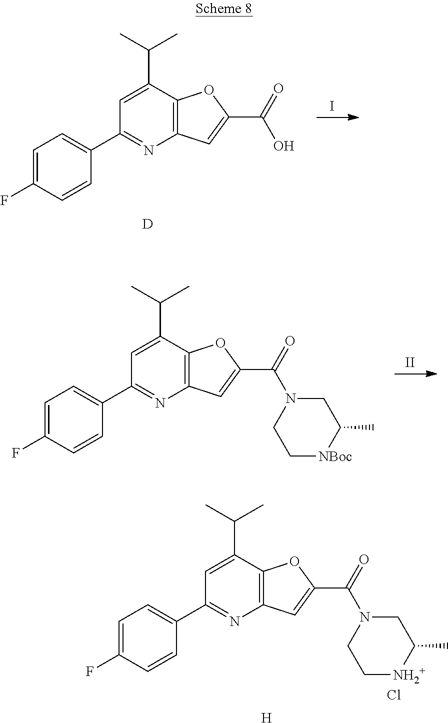

### Step I: tert-Butyl (2S)-4-[5-(4-fluorophenyl)-7-isopropyl-furo[3,2-b]pyridine-2-carbonyl]-2-methyl-piperazine-1-carboxylate

The product was prepared according to General Procedure 1 using Intermediate D (537 mg, 1.80 mmol), HATU (888 mg, 2.33 mmol), DMF (9 mL), Hünig's base (938 μL, 5.39 mmol) and tert-butyl (2S)-2-methylpiperazine-1-carboxylate (396 mg. 1.98 mmol) affording the title compound tert-Butyl (2S)-4-[5-(4-fluorophenyl)-7-isopropyl-furo[3,2-b]pyridine-2-carbonyl]-2-methyl-piperazine-1-carboxylate. (864 mg, 100% yield). ESI-MS m/z calc. 481.23768, found 482.37 (M+1)⁺; Retention time: 1.95 minutes using method C.

### Step II: [5-(4-Fluorophenyl)-7-isopropyl-furo[3,2-b]pyridin-2-yl]-[(3S)-3-methylpiperazin-1-yl]methanone hydrochloride (Intermediate H)

The product was prepared according to General Procedure 2 using tert-butyl (2S)-4-[5-(4-fluorophenyl)-7-isopropyl-furo[3,2-b]pyridine-2-carbonyl]-2-methyl-piperazine-1-carboxylate (864 mg, 1.80 mmol), DCM (17 mL) and HCl in dioxane (4.49 mL of 4 M, 17.95 mmol) affording the title compound. [5-(4-Fluorophenyl)-7-isopropyl-furo[3,2-b]pyridin-2-yl]-[(3S)-3-methylpiperazin-1-yl]methanone hydrochloride (750 mg, 100% yield). ESI-MS m/z calc. 381.18526, found 382.41 (M+1)⁺; Retention time: 1.11 minutes using method C.

### Preparation of Intermediate I: [5-(4-Fluorophenyl)-7-(l-methylcyclopropyl)furo[3,2-b]pyridin-2-yl]-[(3S)-3-methylpiperazin-1-yl]methanone hydrochloride

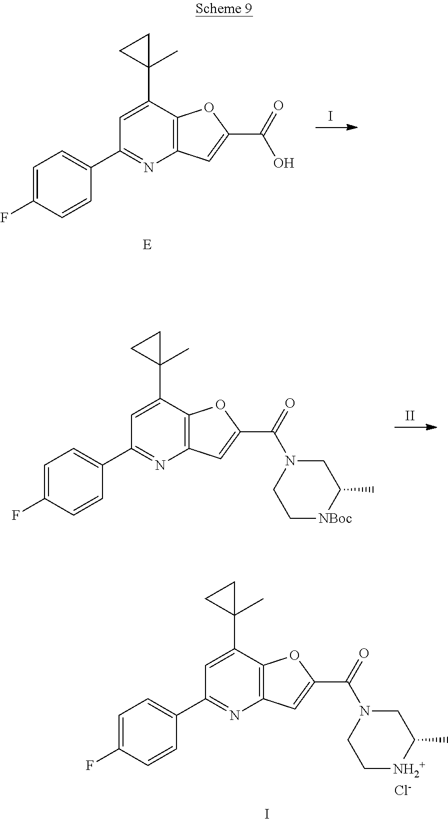

### Step I: [5-(4-Fluorophenyl)-7-(1-methylcyclopropyl)furo[3,2-b]pyridin-2-yl]-[(3S)-3-methylpiperazin-1-yl]methanone hydrochloride

The product was prepared according to General Procedure 1 using Intermediate E (550 mg, 1.77 mmol), HATU (873 mg, 2.30 mmol), DMF (9.3 mL), Hünig's base (1.08 mL, 6.18 mmol) and tert-butyl (2S)-2-methylpiperazine-1-carboxylate (407 mg, 2.03 mmol) affording the title compound. (2S)-4-[5-(4-Fluorophenyl)-7-(1-methylcyclopropyl)furo[3,2-b]pyridine-2-carbonyl]-2-methyl-piperazine-1-carboxylate (870 mg, 100% yield). ESI-MS m/z calc. 493.23767, found 494.73 (M+1)⁺; Retention time: 1.96 minutes using method C.

### Step II: [5-(4-Fluorophenyl)-7-(1-methylcyclopropyl)furo[3,2-b]pyridin-2-yl]-[(3S)-3-methylpiperazin-1-yl]methanone hydrochloride (Intermediate I)

The product was prepared according to General Procedure 2 using (2S)-4-[5-(4-fluorophenyl)-7-(1-methylcyclopropyl)furo[3,2-b]pyridine-2-carbonyl]-2-methyl-piperazine-1-carboxylate (870 mg, 1.76 mmol), DCM (19 mL) and HCl in dioxane (8.82 mL of 4 M, 35.26 mmol) affording the title compound. [5-(4-Fluorophenyl)-7-(1-methylcyclopropyl)furo[3,2-b]pyridin-2-yl]-[(3S)-3-methylpiperazin-1-yl]methanone hydrochloride (753 mg, 99% yield). ESI-MS m/z calc. 393.1853, found 394.36 (M+1); Retention time: 1.14 minutes using method C.

### Preparation of Intermediate J: [5-(4-Fluorophenyl)-7-isopropyl-furo[3,2-b]pyridin-2-yl]-piperazin-1-yl-methanone hydrochloride

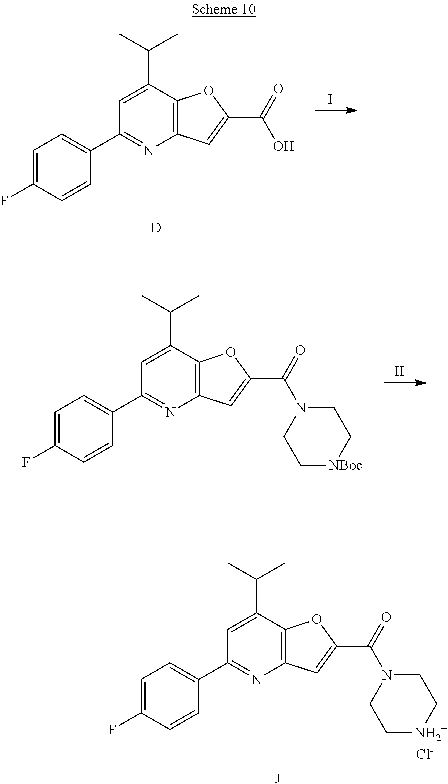

### Step I: tert-Butyl 4-[5-(4-fluorophenyl)-7-isopropyl-furo[3,2-b]pyridine-2-carbonyl]piperazine-carboxylate

The product was prepared according to General Procedure 1 using Intermediate D (219 mg, 0.73 mmol), HATU (361 mg, 0.95 mmol), DMF (3.7 mL), Hünig's base (381 μL, 2.19 mmol) and tert-butyl piperazine-1-carboxylate (150 mg, 0.80 mmol) affording the title compound. tert-Butyl 4-[5-(4-fluorophenyl)-7-isopropyl-furo[3,2-b]pyridine-2-carbonyl]piperazine-1-carboxylate (319 mg 93% yield). 20 mg of the crude product are purified by mass-directed reverse phase HPLC affording the title compound, the remaining product was used in the subsequent step without further purification. ¹H NMR (400 MHz, Chloroform-d) δ 8.00-7.94 (m, 2H), 7.54 (s, 1H), 7.47 (s, 1H), 7.20-7.13 (m, 2H), 3.87-3.75 (m, 4H), 3.62-3.53 (m, 4H), 3.49-3.38 (m, 1H), 1.49 (s, 9H), 1.45 (d, J=6.9 Hz, 6H). ESI-MS m/z calc. 467.22203, found 468.37 (M+1)⁺; Retention time: 1.89 minutes using method C.

### Step II: [5-(4-Fluorophenyl)-7-isopropyl-furo[3,2-b]pyridin-2-yl]-piperazin-1-yl-methanone hydrochloride (Intermediate J)

The product was prepared according to General Procedure 2 using tert-butyl 4-[5-(4-fluorophenyl)-7-isopropyl-furo[3,2-b]pyridine-2-carbonyl]piperazine-1-carboxylate (300 mg, 0.64 mmol), DCM (6.4 mL) and HCl in dioxane (1.60 mL of 4 M, 6.42 mmol) affording the title compound. [5-(4-Fluorophenyl)-7-isopropyl-furo[3,2-b]pyridin-2-yl]-piperazin-1-yl-methanone hydrochloride (259 mg, 100% yield). ESI-MS m/z calc. 367.16961, found 368.32 (M+1)⁺; Retention time: 1.04 minutes using method C.

### Preparation of Intermediate K: [5-(4-Fluorophenyl)-7-isopropyl-furo[3,2-b]pyridin-2-yl]-[(2R)-2-methylpiperazin-1-yl]methanone hydrochloride

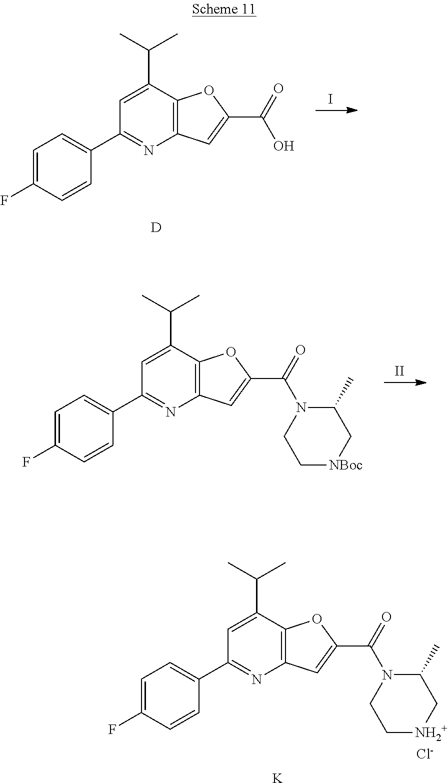

### Step I: tert-Butyl (3R)-4-[S-(4-fluorophenyl)-7-isopropyl-furo[3,2-b]pyridine-2-carbonyl]-3-methyl-piperazine-1-carboxylate

The product was prepared according to General Procedure 1 using Intermediate D (219 mg, 0.73 mmol), HATU (361 mg, 0.95 mmol), DMF (3.7 mL), Hünig's base (381 μL, 2.19 mmol) and tert-butyl (3R)-3-methylpiperazine-1-carboxylate (161 mg, 0.80 mmol) affording the title compound. tert-Butyl (3R)-4-[5-(4-fluorophenyl)-7-isopropyl-furo[3,2-b]pyridine-2-carbonyl]-3-methyl-piperazine-1-carboxylate (332 mg, 94% yield). 20 mg of the crude product are purified by mass-directed reverse phase HPLC affording the title compound, the remaining product was used in the subsequent step without further purification. ¹H NMR (400 MHz, Chloroform-d) δ 8.03-7.93 (m, 2H), 7.54 (s, 1H), 7.45 (s, 1H), 7.23-7.13 (m, 2H), 5.00-3.85 (m, 4H), 3.57-2.87 (m, 4H), 1.58-1.35 (m, 18H). ESI-MS m/z calc. 481.23768, found 482.09 (M+1)⁺; Retention time: 1.95 minutes using method C.

### Step II: [5-(4-Fluorophenyl)-7-isopropyl-furo[3,2-b]pyridin-2-yl]-[(2R)-2-methylpiperazin-1-yl]methanone hydrochloride (Intermediate K)

The product was prepared according to General Procedure 2 using tert-butyl (3R)-4-[5-(4-fluorophenyl)-7-isopropyl-furo[3,2-b]pyridine-2-carbonyl]-3-methyl-piperazine-1-carboxylate (310 mg, 0.64 mmol), DCM (6.4 mL) and HCl in dioxane (1.61 mL of 4 M, 6.44 mmol) affording the title compound. [5-(4-Fluorophenyl)-7-isopropyl-furo[3,2-b]pyridin-2-yl]-[(2R)-2-methylpiperazin-1-yl]methanone hydrochloride (269 mg, 100% yield). ESI-MS m/z calc. 381.18526, found 382.37 (M+1)⁺; Retention time: 1.13 minutes using method C.

### Preparation of Intermediate L: [5-(4-Fluorophenyl)-7-isopropyl-furo[3,2-b]pyridin-2-yl]-[(2S)-2-methylpiperazin-1-yl]methanone hydrochloride

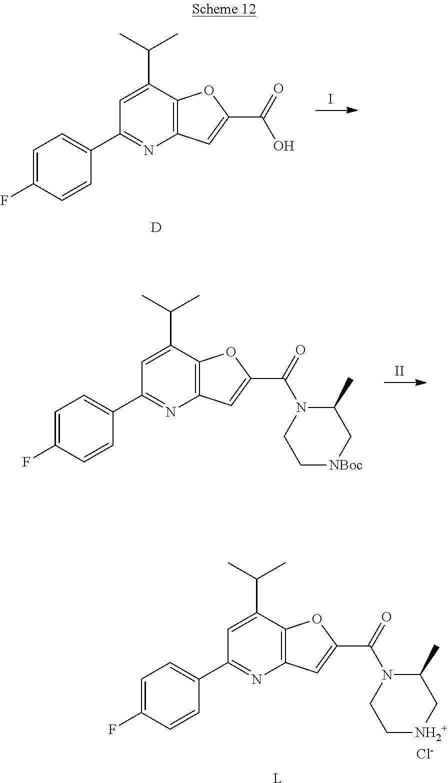

### Step I: tert-Butyl (3S)-4-[5-(4-fluorophenyl)-7-isopropyl-furo[3,2-b]pyridine-2-carbonyl]-3-methyl-piperazine-1-carboxylate

The product was prepared according to General Procedure 1 using Intermediate D (219 mg, 0.73 mmol), HATU (361 mg, 0.95 mmol), DMF (3.7 mL), Hünig's base (381 μL, 2.19 mmol) and tert-butyl (3S)-3-methylpiperazine-1-carboxylate (161 mg, 0.80 mmol) affording the title compound. tert-Butyl (3S)-4-[5-(4-fluorophenyl)-7-isopropyl-furo[3,2-b]pyridine-2-carbonyl]-3-methyl-piperazine-1-carboxylate (351 mg, 100% yield). 20 mg of the crude product are purified by mass-directed reverse phase HPLC affording the title compound, the remaining product was used in the subsequent step without further purification. ¹H NMR (400 MHz, Chloroform-d) δ 8.07-7.92 (m, 2H), 7.54 (s, 1H), 7.45 (s, 1H), 7.23-7.10 (m, 2H), 4.84-3.86 (m, 4H), 3.55-2.86 (m, 4H), 1.77-1.18 (m, 18H). ESI-MS m/z calc. 481.23768, found 482.13 (M+1)⁺; Retention time: 1.95 minutes using method C.

### Step II: [5-(4-Fluorophenyl)-7-isopropyl-furo[3,2-b]pyridin-2-yl]-[(2S)-2-methylpiperazin-1-yl]methanone hydrochloride (Intermediate L)

The product was prepared according to General Procedure 2 using tert-butyl (3S)-4-[5-(4-fluorophenyl)-7-isopropyl-furo[3,2-b]pyridine-2-carbonyl]-3-methyl-piperazine-1-carboxylate (330 mg, 0.69 mmol), DCM (7 mL) and HCl in dioxane (1.71 mL of 4 M, 6.85 mmol) affording the title compound. [5-(4-Fluorophenyl)-7-isopropyl-furo[3,2-b]pyridin-2-yl]-[(2S)-2-methylpiperazin-1-yl]methanone hydrochloride (286 mg, 100% yield). ESI-MS m/z calc. 381.18526, found 382.37 (M+1)⁺; Retention time: 1.12 minutes using method C.

### Preparation of Intermediate M: 2-(4-Fluorophenyl)-4-isopropyl-furo[3,2-d]pyrimidine-6-carboxylic acid

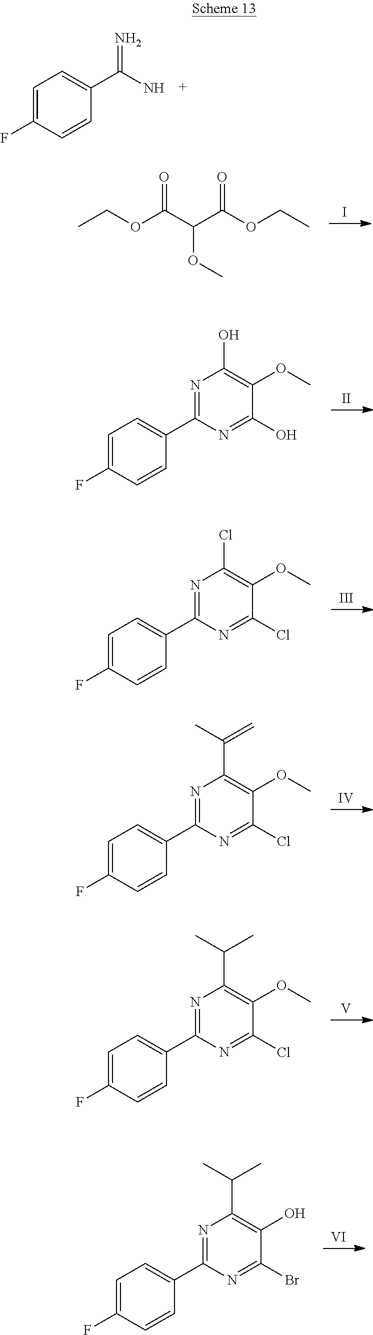

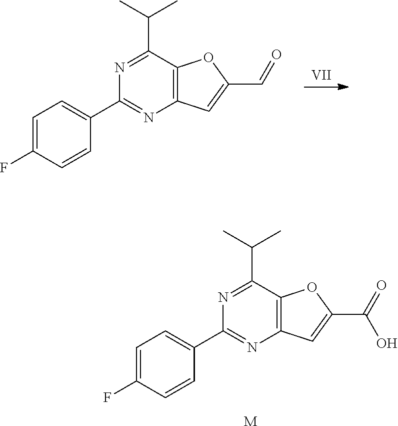

### Step I: 2-(4-Fluorophenyl)-5-methoxy-pyrimidine-4,6-diol

To a solution of 4-fluorobenzamidine hydrochloride (1.00 g, 5.73 mmol) in EtOH (10 mL) were added diethyl 2-methoxypropanedioate (970 μL, 5.73 mmol) and 21% wt. NaOEt in EtOH (5.00 mL, 63.8 mmol) and the solution was stirred at 78° C. for 16 h. The volatiles were removed under reduced pressure and the residue was dissolved in 10 mL of hot water. Then the solution was acidified with 12N HCl and the precipitate formed collected by filtration. The solid was washed with water and dried under reduced pressure affording the title compound. 2-(4-Fluorophenyl)-5-methoxy-pyrimidine-4,6-diol (1.13 g, 83% yield). ¹H NMR (400 MHz, DMSO-d6) δ 8.21-7.99 (m, 2H), 7.32 (t, J=8.9 Hz, 2H), 3.65 (s, 3H).

### Step II: 4,6-Dichloro-2-(4-fluorophenyl)-5-methoxy-pyrimidine

To a mixture of 2-(4-fluorophenyl)-5-methoxy-pyrimidine-4,6-diol (1.13 g, 4.76 mmol) in POCl₃ (6.00 mL, 64.37 mmol) was added N,N-dimethylaniline (600.0 μL, 4.73 mmol) and the resulting solution was stirred at 106° C. for 4 h. The volatiles were removed under reduced pressure and a mixture of ice/water was added to the residue. The solution was neutralized with sat. aq. NaHCO₃ and extracted with DCM. The combined organic extracts were washed with water and brine, dried over Na₂SO₄, filtered and the filtrate concentrated under reduced pressure. The residue was purified by flash chromatography on silica gel eluting with 0-35% DCM: Hexanes affording the title compound. 4,6-Dichloro-2-(4-fluorophenyl)-5-methoxy-pyrimidine (1.15 g, 89% yield). ¹H NMR (400 MHz, Chloroform-d) δ 8.36 (dd, J=9.0, 5.5 Hz, 2H), 7.13 (dd, J=9.0, 8.4 Hz, 2H), 3.98 (s, 3H).

### Step III: 4-Chloro-2-(4-fluorophenyl)-6-isopropenyl-5-methoxy-pyrimidine

To a solution of 4,6-dichloro-2-(4-fluorophenyl)-5-methoxy-pyrimidine (1.00 g, 3.66 mmol) in dioxane (10 mL) were added 2-isopropenyl-4,4,5,5-tetramethyl-1,3,2-dioxaborolane (800 μL, 4.26 mmol), Pd(dppf)Cl₂-DCM (150 mg, 0.18 mmol) and aq. Na₂CO₃ (3.70 mL of 2M, 7.40 mmol) and the solution was degassed with nitrogen. The solution was heated at 100° C. under a nitrogen atmosphere for 2 h, cooled to room temperature and diluted with EtOAc. The organic phase was washed with water and brine and dried over Na₂SO₄, filtered and the filtrate concentrated under reduced pressure. The residue was purified by flash chromatography on silica gel eluting with 0-25% DCM: Hexanes affording the title compound. 4-chloro-2-(4-fluorophenyl)-6-isopropenyl-5-methoxy-pyrimidine (600.0 mg, 59% yield). ¹H NMR (400 MHz, Chloroform-d) δ 8.38 (dd, J=8.9, 5.6 Hz, 2H), 7.11 (dd, J=9.0, 8.4 Hz, 2H), 6.06 (dd, J=1.6, 0.9 Hz, 1H), 5.77-5.41 (m, 1H), 3.82 (s, 3H), 2.27 (dd, J=1.6, 0.9 Hz, 3H).

### Step IV: 4-Chloro-2-(4-fluorophenyl)-6-isopropyl-5-methoxy-pyrimidine

To a solution of 4-chloro-2-(4-fluorophenyl)-6-isopropenyl-5-methoxy-pyrimidine (820 mg, 2.94 mmol) in MeOH (4 mL) and DCM (4 mL) was added PtO₂ (40 mg, 0.18 mmol). The flask was evacuated under reduced pressure and re-filled with hydrogen 3 times. The suspension was stirred under a hydrogen atmosphere at room temperature for 20 minutes. Hydrogen was evacuated and the flask backfilled with nitrogen. The catalyst was filtered on Celite® and the volatiles were removed under reduced pressure affording the title compound. 4-Chloro-2-(4-fluorophenyl)-6-isopropyl-5-methoxy-pyrimidine (810 mg, 98% yield). ¹H NMR (400 MHz, Chloroform-d) δ 8.56-8.29 (m, 2H), 7.14-6.99 (m, 2H), 3.89 (s, 3H), 3.46 (p, J=6.8 Hz, 1H), 1.31 (d, J=6.8 Hz, 6H).

### Step V: 4-Bromo-2-(4-fluorophenyl)-6-isopropyl-pyrimidin-5-ol

A solution of 4-chloro-2-(4-fluorophenyl)-6-isopropyl-5-methoxy-pyrimidine (100 mg, 0.36 mmol) in 33% HBr/AcOH (1.00 mL, 6.08 mmol) was heated at 100° C. for 2 h. The volatiles were removed under reduced pressure and the residue was purified by flash chromatography on silica gel eluting with 0-25% DCM: Hexanes affording the title compound. 4-Bromo-2-(4-fluorophenyl)-6-isopropyl-pyrimidin-5-ol (56 mg, 51% yield). ¹H NMR (400 MHz, Chloroform-d) δ 8.42-8.23 (m, 2H), 7.16-6.96 (m, 2H), 5.42 (s, 1H), 3.45 (p. J=6.8 Hz, 1H), 1.32 (d, J=6.8 Hz, 6H).

### Step VI: 2-(4-Fluorophenyl)-4-isopropyl-furo[3,2-d]pyrimidine-6-carbaldehyde

To a solution of 4-bromo-2-(4-fluorophenyl)-6-isopropyl-pyrimidin-5-ol (40 mg, 0.13 mmol) in DMF (3 mL) were added Pd(dppf)Cl₂-DCM (10.5 mg, 0.013 mmol). CuI (3.7 mg, 0.019 mmol) and Hünig's base (67 μL, 0.38 mmol) and the solution was degassed with nitrogen. The solution was stirred at 100° C. under nitrogen for 2 h, cooled to room temperature and diluted with EtOAc. The organic phase was washed with water and brine and dried over Na₂SO₄, filtered and the filtrate concentrated under reduced pressure. The residue was diluted in TFA (1.00 mL) and the solution stirred at room temperature for 30 min. The volatiles were removed under reduced pressure and the residue was purified by flash chromatography on silica gel eluting with 0-15% EtOAc: Hexanes affording the title compound. 2-(4-Fluorophenyl)-4-isopropyl-furo[3,2-d]pyrimidine-6-carbaldehyde (11.5 mg, 31% yield). ¹H NMR (400 MHz, Chloroform-d) δ 10.04 (s, 1H), 8.70-8.31 (m, 2H), 7.67 (s, 1H), 7.19-6.95 (m, 2H), 3.66 (p, J=6.9 Hz, 1H), 1.50 (d, J=6.9 Hz, 7H).

### Step VII: 2-(4-Fluorophenyl)-4-isopropyl-furo[3,2-d]pyrimidine-6-carboxylic acid (Intermediate M)

To a solution of 2-(4-fluorophenyl)-4-isopropyl-furo[3,2-d]pyrimidine-6-carbaldehyde (46 mg, 0.16 mmol) in acetonitrile (6 mL) and water (2 mL) were added NaH₂PO₄ (58 mg, 0.49 mmol) and NaClO₂ (55 mg, 0.49 mmol). The resulting solution was stirred at room temperature for 1 h. The solution was diluted with EtOAc and the pH adjusted to 3 using 2N HCl. The organic phase was washed with water and brine, dried over Na₂SO₄, and filtered. The filtrate was concentrated under reduced pressure affording the title compound which is used directly in the next step without further purification. 2-(4-Fluorophenyl)-4-isopropyl-furo[3,2-d]pyrimidine-6-carboxylic acid (48 mg, 99% yield). ¹H NMR (400 MHz, Methanol-d4) δ 8.65-8.40 (m, 2H), 7.64 (s, 1H), 7.30-7.10 (m, 2H), 3.64 (p, J=6.9 Hz; 1H), 1.50 (d, J=6.9 Hz, 6H).

### Preparation of Intermediate N: 2-(4-Fluorophenyl)-4-isopropyl-furo[3,2-d]pyrimidine-6-carboxylic acid

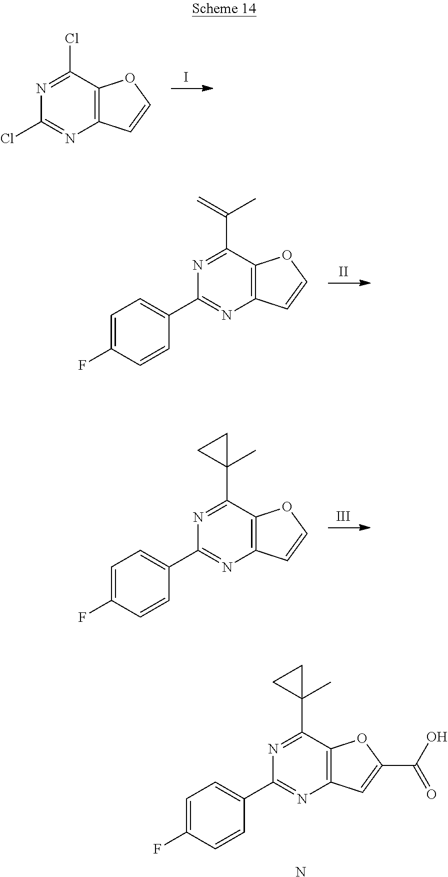

### Step I: 2-(4-Fluorophenyl)-4-isopropenyl-furo[3,2-d]pyrimidine

To a solution of 2,4-dichlorofuro[3,2-d]pyrimidine (509 mg, 2.69 mmol) in dioxane (15 mL) were added 2-isopropenyl-4,4,5,5-tetramethyl-1,3,2-dioxaborolane (524 mg, 2.96 mmol), Pd(dppf)Cl₂-DCM (110 mg, 0.13 mmol) and K₃PO₄ (1.14 g, 5.37 mmol). The resulting solution was degassed with nitrogen. The solution was heated at 100° C. under a nitrogen atmosphere for 16 h before (4-fluorophenyl)boronic acid (427 mg, 2.96 mmol), aq. Na₂CO₃ (2.70 mL of 2M, 5.40 mmol) and Pd(dppf)Cl₂-DCM (110 mg, 0.13 mmol) were added and the solution stirred at 100° C. for another 5 h. The solution was cooled to room temperature, diluted with EtOAc and water, and filtered over Celite®. Phases were separated and the aq. phase washed twice with EtOAc. The combined organic phase was washed with water and brine, dried over Na₂SO₄, and filtered. The filtrate was concentrated under reduced pressure. The residue was purified by flash chromatography on silica gel eluting with 0-50% EtOAc: Hexanes affording the title compound. 2-(4-Fluorophenyl)-4-isopropenyl-furo[3,2-d]pyrimidine (480 mg, 70% yield). ¹H NMR (400 MHz, Chloroform-d) δ 8.64-8.33 (m, 2H), 7.95 (d, J=2.2 Hz, 1H), 7.20-7.08 (m, 2H), 7.01 (d, J=2.3 Hz, 1H), 6.56 (p. J=0.9 Hz, 1H), 5.97-5.76 (m, 1H), 2.41 (dd, J=1.6, 0.9 Hz, 3H).

### Step II: 2-(4-Fluorophenyl)-4-(1-methylcyclopropyl)furo[3,2-d]pyrimidine

To a solution of 2-(4-fluorophenyl)-4-isopropenyl-furo[3,2-d]pyrimidine (480 mg, 1.89 mmol) in DCM (20 mL) was added Pd(OAc)₂ (21.2 mg, 0.094 mmol) and the solution cool to 0° C. A solution of diazomethane (113.4 mL of 1 M in diethyl ether, 113.45 mmol) was added dropwise over 1 h or until the complete consumption of the starting material. The volatiles were removed under reduced pressure and the residue is purified by flash chromatography on silica gel eluting with 0-10% EtOAc: Hexanes affording the title compound. 2-(4-Fluorophenyl)-4-(1-methylcyclopropyl)furo[3,2-d]pyrimidine (410 mg, 81% yield). ¹H NMR (400 MHz, Chloroform-d) δ 8.54-8.36 (m, 2H), 7.83 (d, J=2.3 Hz, 1H), 7.18-7.05 (m, 2H), 6.91 (d, J=2.2 Hz, 1H), 1.75 (s, 3H), 1.65 (q, J=3.9 Hz, 2H), 1.03-0.93 (m, 2H).

### Step II: 2-(4-Fluorophenyl)-4-(1-methylcyclopropyl)furo[3,2-d]pyrimidine-6-carboxylic acid (Intermediate N)

A solution of 2-(4-fluorophenyl)-4-(1-methylcyclopropyl)furo[3,2-d]pyrimidine (100 mg, 0.37 mmol) in THF (4 mL) under nitrogen was cooled to −78° C. before n-BuLi (200 μL of 2.5 M in hexanes, 0.50 mmol). After stirring for 1 h at −78° C., CO₂ gas was bubbled through the solution for 10 min and warmed up to 0° C. over 1 h. 2N HCl is added and the aq. phase extracted with EtOAc. The organic phase was washed with water and brine, dried over Na₂SO₄ and filtered. The filtrate was concentrated under reduced pressure affording the title compound which was used in the subsequent step without further purification. 2-(4-Fluorophenyl)-4-(1-methylcyclopropyl)furo[3,2-d]pyrimidine-6-carboxylic acid (100 mg, 86% yield). ¹H NMR (400 MHz, CD₃OD) δ 8.62-8.41 (m, 2H), 7.59 (s, 1H), 7.27-7.10 (m, 2H), 1.82 (s, 3H), 1.76-1.68 (m, 2H), 1.14-1.05 (m, 2H). ESI-MS m/z calc. 312.09102, found 313.62 (M+1)⁺; Retention time: 3.58 minutes using method A.

### Preparation of Intermediate O: 2-Chloro-6-(4-fluorophenyl)-4-isopropyl-pyridin-3-amine

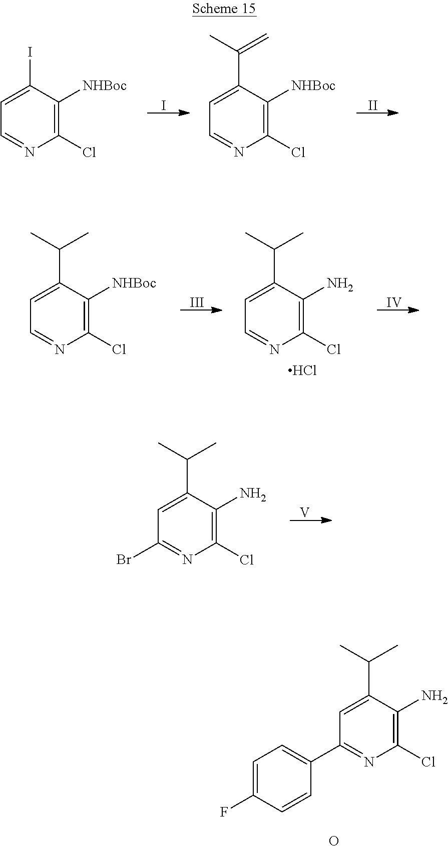

### Step I: tert-Butyl N-(2-chloro-4-isopropenyl-3-pyridyl)carbamate

To a solution of tert-butyl N-(2-chloro-4-iodo-3-pyridyl)carbamate (500 mg, 1.41 mmol) in dioxane (10 mL) were added 2-isopropenyl-4,4,5,5-tetramethyl-1,3,2-dioxaborolane (274 mg, 1.55 mmol), Pd(dppf)Cl₂-DCM (58 mg, 0.071 mmol) and aq. Na₂CO₃ (1.40 mL of 2M, 2.82 mmol). The resulting solution was degassed with nitrogen. The solution was stirred at 80° C. for 5 h, cooled to room temperature and diluted with EtOAc. Phases were separated and the organic phase washed with water and brine consecutively, dried with Na₂SO₄ and filtered. The filtrate was concentrated under reduced pressure. The residue was purified by flash chromatography on silica gel eluting with 0-25% EtOAc: Hexanes affording the title compound. tert-Butyl N-(2-chloro-4-isopropenyl-3-pyridyl)carbamate (278 mg, 73% yield). ¹H NMR (400 MHz. Chloroform-d) δ 8.18 (d, J=5.0 Hz, 1H), 7.09 (d, J=5.0 Hz, 1H), 6.06 (s, 1H), 5.23 (p, J=1.5 Hz, 1H), 5.08 (p, J=1.0 Hz, 1H), 2.06 (dd, J=1.6, 0.9 Hz, 3H), 1.46 (s, 9H).

### Step II: tert-Butyl N-(2-chloro-4-isopropyl-3-pyridyl)carbamate

To a solution of tert-butyl N-(2-chloro-4-isopropenyl-3-pyridyl)carbamate (278 mg, 1.03 mmol) in MeOH (5 mL) and DCM (2 mL) was added Pd/C 10 wt. % (30 mg, 0.028 mmol). The flask was evacuated under reduced pressure and re-filled with hydrogen 3 times and the suspension was stirred under a hydrogen atmosphere at room temperature for 30 minutes. Hydrogen was evacuated and the flask backfilled with nitrogen. The catalyst was filtered on Celite® and the volatiles were removed under reduced pressure. The residue was purified by flash chromatography on silica gel eluting with 0-25% EtOAc: Hexanes affording the title compound. tert-Butyl N-(2-chloro-4-isopropyl-3-pyridyl)carbamate (134 mg, 48% yield). ¹H NMR (400 MHz. Chloroform-d) δ 8.21 (d, J=5.1 Hz, 1H), 7.17 (d, J=5.2 Hz, 1H), 5.97 (s, 1H), 3.24 (s, 1H), 1.48 (s, 9H), 1.21 (d, J=6.9 Hz 6H).

### Step III: 2-Chloro-4-isopropyl-pyridin-3-amine hydrochloride

To a solution of tert-butyl N-(2-chloro-4-isopropyl-3-pyridyl)carbamate (134 mg, 0.49 mmol) in DCM (1 mL) was added HCl in dioxane (1 mL of 4 M, 4 mmol). The solution was stirred at room temperature for 1 h. Upon reaction completion, the volatiles were removed under reduced pressure affording the crude product which was used in the subsequent step without further purification. 2-Chloro-4-isopropyl-pyridin-3-amine hydrochloride (103 mg, 100% yield). ESI-MS m/z calc. 170.06108, found 171.10 (M+1)⁺; Retention time: 1.78 minutes using method A.

### Step IV: 6-Bromo-2-chloro-4-isopropyl-pyridin-3-amine

To a solution of 2-chloro-4-isopropyl-pyridin-3-amine hydrochloride (683 mg, 3.30 mmol) in acetonitrile (15 mL) was added 1,3-dibromo-5,5-dimethyl-imidazolidine-2,4-dione (755 mg, 2.64 mmol). The solution was stirred at room temperature for 20 min. Upon completion, the reaction mixture was diluted with EtOAc. The organic phase was washed with a saturated aq. Na₂S₂O₃ solution, water and brine, dried over Na₂SO₄ and filtered. The filtrate was concentrated under reduced pressure. The residue was purified by flash chromatography on silica gel eluting with 0-30% EtOAc: Hexanes affording the title compound. 6-Bromo-2-chloro-4-isopropyl-pyridin-3-amine (444 mg, 54% yield). ¹H NMR (400 MHz, Chloroform-d) δ 7.09 (d, J=0.6 Hz, 1H), 4.12 (s, 2H), 2.81 (pd, J=6.8, 0.6 Hz, 1H), 1.22 (dd, J=6.8, 1.5 Hz, 6H).

### Step V: 2-Chloro-6-(4-fluorophenyl)-4-isopropyl-pyridin-3-amine (Intermediate O)

To a solution of 6-bromo-2-chloro-4-isopropyl-pyridin-3-amine (100 mg, 0.40 mmol) in dioxane (3 mL) were added (4-fluorophenyl)boronic acid (63 mg, 0.44 mmol). Pd(dppf)Cl₂-DCM (16 mg, 0.020 mmol) and aq. Na₂CO₃ (400 μL of 2M, 0.80 mmol). The solution was degassed with nitrogen, and then stirred at 80° C. for 5 h. Upon completion the reaction mixture was cooled to room temperature and diluted with EtOAc. Phases were separated and the organic phase washed with water and brine consecutively, dried with Na₂SO₄ and filtered. The filtrate was concentrated under reduced pressure. The residue was purified by flash chromatography on silica gel eluting with 0-25% EtOAc: Hexanes affording the title compound. 2-Chloro-6-(4-fluorophenyl)-4-isopropyl-pyridin-3-amine (83 mg, 78% yield). ¹H NMR (400 MHz, Chloroform-d) δ 7.92-7.75 (m, 2H), 7.37 (s, 1H), 7.13-7.02 (m, 2H), 4.13 (s, 2H), 2.89 (pd, J=6.8, 0.5 Hz, 1H), 1.30 (d, J=6.8 Hz, 6H).

### Preparation of Intermediate P: 5-(4-Fluorophenyl)-7-isopropyl-thiazolo[5,4-b]pyridine-2-carboxylic acid

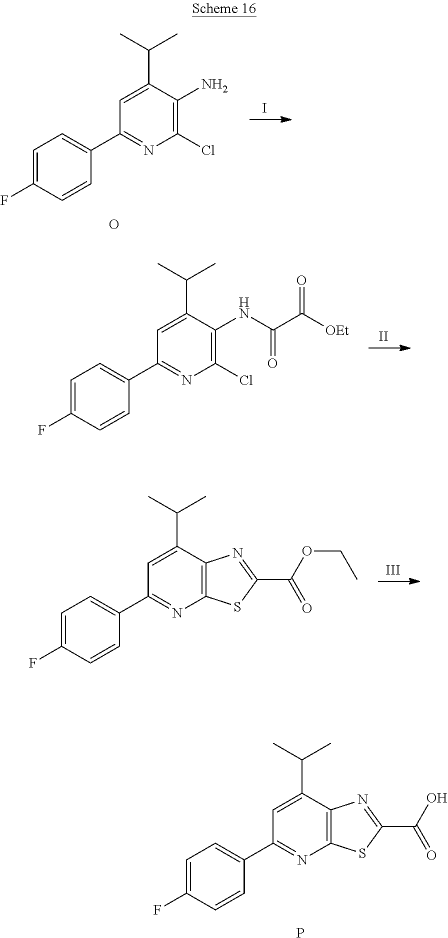

### Step I: Ethyl 5-(4-fluorophenyl)-7-isopropyl-thiazolo[5,4-b]pyridine-2-carboxylate

To a solution of Intermediate 0 (83 mg, 0.31 mmol) and 2,6-lutidine (101 mg, 109 μL, 0.94 mmol) in DCM (4 mL) was added ethyl 2-chloro-2-oxo-acetate (43 mg. 0.31 mmol) at 0° C., and the solution is stirred at room temperature for 1 h. The solution was diluted with DCM and the organic phase washed with sat. aq. NaHCO₃, water and brine, dried over Na₂SO₄ and filtered. The filtrate was concentrated under reduced pressure affording the desired compound which was used in the subsequent step without further purification. Ethyl 2-[[2-chloro-6-(4-fluorophenyl)-4-isopropyl-3-pyridyl]amino]-2-oxo-acetate (120 mg, 100% yield). ESI-MS m/z calc. 364.09900, found 365.57 (M+1)⁺; Retention time: 3.19 minutes using method A.

### Step II: Ethyl 5-(4-fluorophenyl)-7-isopropyl-thiazolo[5,4-b]pyridine-2-carboxylate

To a solution of ethyl 2-[[2-chloro-6-(4-fluorophenyl)-4-isopropyl-3-pyridyl]amino]-2-oxo-acetate (120 mg, 0.33 mmol) in toluene (4 mL) was added Lawesson's reagent (127 mg, 0.31 mmol) and the solution was stirred at 110° C. for 1 h. The volatiles were removed under reduced pressure and the residue was purified by flash chromatography on silica gel eluting with 0-40% EtOAc: Hexanes affording the title compound. Ethyl 5-(4-fluorophenyl)-7-isopropyl-thiazolo[5,4-b]pyridine-2-carboxylate (100 mg, 88% yield). ¹H NMR (400 MHz, Chloroform-d) δ 8.15-7.91 (m, 2H), 7.60 (s. 1H), 7.19-6.98 (m, 2H), 4.46 (q, J=7.1 Hz, 2H), 3.04-2.85 (m, 1H), 1.47 (t, J=7.1 Hz, 3H), 1.25 (broad s, 6H).

### Step III: 5-(4-Fluorophenyl)-7-isopropyl-thiazolo[5,4-b]pyridine-2-carboxylic acid (Intermediate P)

To a solution of ethyl 5-(4-fluorophenyl)-7-isopropyl-thiazolo[5,4-b]pyridine-2-carboxylate (80 mg, 0.23 mmol) in MeOH (3 mL) and water (1 mL) was added LiOH hydrate (20 mg, 0.46 mmol) and the solution was heated at 60° C. for 2 h. The solution was cooled to room temperature, neutralized with resin Amberlite IR 120-H and filtered. The filtrate was concentrated under reduced pressure affording the title compound which was used in the subsequent step without further purification. 5-(4-Fluorophenyl)-7-isopropyl-thiazolo[5,4-b]pyridine-2-carboxylic acid (72 mg, 98% yield). ¹H NMR (400 MHz, Methanol-d4) δ 8.13-8.00 (m, 2H), 7.81 (s, 1H), 7.27-7.15 (m, 2H), 3.07-2.95 (m, 1H), 1.28 (t, J=6.6 Hz, 6H)

### Preparation of Intermediate Q: 5-(4-Fluorophenyl)-7-isopropyl-oxazolo[5,4-b]pyridine-2-carboxylic acid

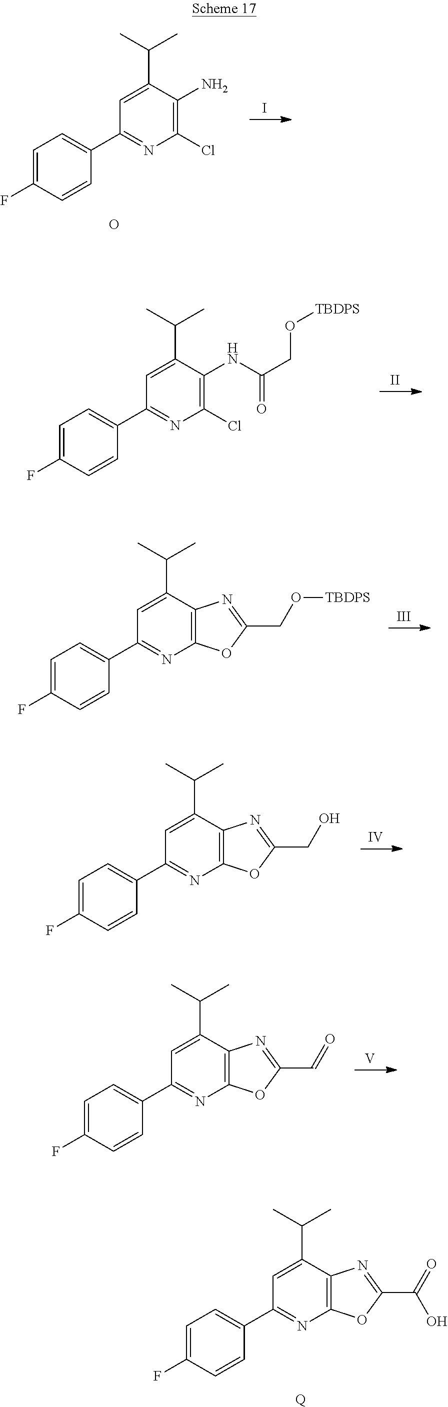

### Step I: 2-[tert-Butyl(diphenyl)sily]oxy-N-[2-chloro-6-(4-fluorophenyl)-4-isopropyl-3-pyridyl]acetamide

To a solution of 2-[tert-butyl(diphenyl)silyl]oxyacetic acid (412 mg, 1.28 mmol) in DCM (10 mL) at 0° C. was added oxalyl chloride (1.20 mL of 2M in DCM, 2.40 mmol) followed by a drop of DMF (10 μL) and the solution was stirred at room temperature for 1 h. The volatiles were removed under reduced pressure and co-evaporated with DCM. The residue was dissolved in DCM (2 mL) and added to a solution of Intermediate O (170 mg, 0.64 mmol) in DCM (5 mL) at 0° C. Hünig's base (210 μL, 1.21 mmol) was added and the solution was stirred at room temperature for 1 h. The volatiles were removed under reduced pressure and the residue was purified by flash chromatography on silica gel eluting with 0-10% EtOAc: Hexanes affording the title compound. 2-[tert-Butyl(diphenyl)silyl]oxy-N-[2-chloro-6-(4-fluorophenyl)-4-isopropyl-3-pyridyl]acetamide (300 mg, 83% yield). ¹H NMR (400 MHz, Chloroform-d) δ 8.43 (s, 1H), 8.21-7.92 (m, 2H), 7.77-7.67 (m, 4H), 7.57 (s, 1H), 7.53-7.36 (m, 6H), 7.20-7.05 (m, 2H), 4.35 (s, 2H), 3.23-3.00 (m, 1H), 1.29 (d, J=7.0 Hz, 6H), 1.16 (s, 9H).

### Step II: tert-Butyl-[[5-(4-fluorophenyl)-7-isopropyl-oxazolo[5,4-b]pyridin-2-yl]methoxy]-diphenyl-silane

To a solution of 2-[tert-butyl(diphenyl)silyl]oxy-N-[2-chloro-6-(4-fluorophenyl)-4-isopropyl-3-pyridyl]acetamide (300 mg, 0.53 mmol) in THF (5 mL) were added CuI (20 mg, 0.11 mmol), Cs₂CO₃ (261 mg, 0.80 mmol) and 1,10-phenanthroline (19 mg, 0.11 mmol). The solution was heated in a microwave reactor at 140° C. for 1 h. The volatiles were removed under reduced pressure and the residue was purified by flash chromatography on silica gel eluting with 0-10% EtOAc: Hexanes affording the title compound. tert-Butyl-[[5-(4-fluorophenyl)-7-isopropyl-oxazolo[5,4-b]pyridin-2-yl]methoxy]-diphenyl-silane (184 mg, 66% yield). ¹H NMR (400 MHz, Chloroform-d) δ 8.17-7.88 (m, 2H), 7.85-7.68 (m, 4H), 7.55 (d, J=0.6 Hz, 1H), 7.48-7.31 (m, 6H), 7.21-7.03 (m, 2H), 4.92 (s, 2H), 3.66-3.27 (m, 1H), 1.43 (d, J=6.9 Hz, 6H), 1.10 (s, 9H).

### Step III: [5-(4-Fluorophenyl)-7-isopropyl-oxazolo[5,4-b]pyridin-2-yl]methanol

To a solution of tert-butyl-[[5-(4-fluorophenyl)-7-isopropyl-oxazolo[5,4-b]pyridin-2-yl]methoxy]-diphenyl-silane (184 mg, 0.35 mmol) in THF (2 mL) was added TBAF (400 μL of 1M in THF, 0.40 mmol). The solution was stirred at room temperature for 10 min. The volatiles were removed under reduced pressure and the residue was purified by flash chromatography on silica gel eluting with 20-80% EtOAc: Hexanes affording the title compound. [5-(4-Fluorophenyl)-7-isopropyl-oxazolo[5,4-b]pyridin-2-yl]methanol (95 mg, 95% yield). ¹H NMR (400 MHz, Chloroform-d) δ 8.25-7.87 (m, 2H), 7.56 (s, 1H), 7.18-6.84 (m, 2H), 4.94 (s, 2H), 3.55 (p, J=6.9 Hz, 1H), 1.43 (d, J=6.9 Hz, 6H).

### Step IV: 5-(4-Fluorophenyl)-7-isopropyl-oxazolo[5,4-b]pyridine-2-carbaldehyde

To a solution of [5-(4-fluorophenyl)-7-isopropyl-oxazolo[5,4-b]pyridin-2-yl]methanol (90 mg, 0.31 mmol) in DCM (2 mL) was added PCC (203 mg, 0.94 mmol). The solution was stirred at room temperature for 5 h. The crude mixture was purified by flash chromatography on silica gel eluting with 0-40% EtOAc: Hexanes affording the title compound. 5-(4-Fluorophenyl)-7-isopropyl-oxazolo[5,4-b]pyridine-2-carbaldehyde (40 mg, 45% yield). ¹H NMR (400 MHz. Chloroform-d) δ 9.97 (s, 1H), 8.27-7.94 (m, 2H), 7.72 (d, J=0.7 Hz, 1H), 7.21-7.00 (m, 2H), 3.62 (ddd, J=13.9, 7.1, 6.5 Hz, 1H), 1.49 (d, J=6.9 Hz, 6H).

### Step V: 5-(4-Fluorophenyl)-7-isopropyl-oxazolo[5,4-b]pyridine-2-carboxylic acid (Intermediate Q)

To a solution of 5-(4-fluorophenyl)-7-isopropyl-oxazolo[5,4-b]pyridine-2-carbaldehyde (40 mg, 0.14 mmol) in acetonitrile (3 mL) and water (1 mL) were added NaH₂PO₄ (51 mg, 0.42 mmol) and NaClO₂ (48 mg, 0.42 mmol). The solution is stirred at room temperature for 1 h. The solution was diluted with EtOAc and the pH adjusted to 3 using 2N HCl. The organic phase was washed with water and brine, dried over Na₂SO₄ and filtered. The filtrate was concentrated under reduced pressure affording the title compound which was used directly in the next step without further purification. 5-(4-Fluorophenyl)-7-isopropyl-oxazolo[5,4-b]pyridine-2-carboxylic acid (35 mg, 83% yield). ESI-MS m/z calc. 300.09102, found 301.27 (M+1)⁺; Retention time: 2.32 minutes using method A.

### Preparation of Intermediate R: (2R)-1-(3,3-Dimethylpiperazin-1-yl)-2-hydroxy-4,4-dimethyl-pentan-1-one hydrochloride

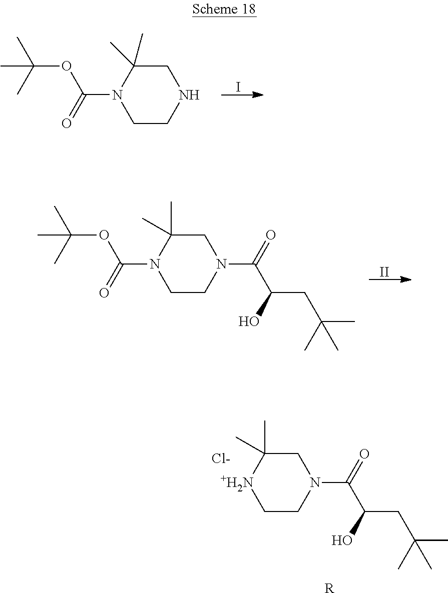

### Step I: tert-Butyl 4-[(2R)-2-hydroxy-4,4-dimethyl-pentanoyl]-2,2-dimethyl-piperazine-1-carboxylate

To a solution of (2R)-2-hydroxy-4,4-dimethyl-pentanoic acid (100 mg, 0.68 mmol) in DMF (2 mL) were added tert-butyl 2,2-dimethylpiperazine-1-carboxylate (176 mg, 0.82 mmol), HATU (338 mg, 0.89 mmol) and Hünig's base (360 μL, 2.05 mmol). The solution was stirred at room temperature overnight. The solution was diluted with EtOAc and the organic phase washed with water and brine, dried over Na₂SO₄ and filtered. The filtrate was concentrated under reduced pressure. The residue was purified by flash chromatography on silica gel eluting with 0-50% EtOAc: Hexanes affording the title compound. tert-Butyl 4-[(2R)-2-hydroxy-4,4-dimethyl-pentanoyl]-2,2-dimethyl-piperazine-1-carboxylate (130 mg, 55% yield). ¹H NMR (400 MHz, Methanol-d4) δ 4.48 (dt, J=8.7, 3.4 Hz, 1H), 3.86-3.37 (m, 6H), 1.46 (s, 11H), 1.42 (d, J=3.9 Hz, 3H), 1.37 (d, J=7.5 Hz, 3H), 1.00 (d, J=2.0 Hz; 9H).

### Step II: (2R)-1-(3,3-Dimethylpiperazin-1-yl)-2-hydroxy-4,4-dimethyl-pentan-1-one hydrochloride (Intermediate R)

To a solution of tert-butyl 4-[(2R)-2-hydroxy-4,4-dimethyl-pentanoyl]-2,2-dimethyl-piperazine-1-carboxylate (130 mg) in DCM (1 mL) was added 4N HCl in dioxane (1.00 mL, 4.00 mmol). The solution was stirred at room temperature for 1 h. The volatiles were removed under reduced pressure affording the title compound which was used directly in the subsequent step without further purification. (2R)-1-(3,3-Dimethylpiperazin-1-yl)-2-hydroxy-4,4-dimethyl-pentan-1-one hydrochloride (105 mg, quantitative yield).

### Preparation of Intermediate S: (2R)-2-Hydroxy-4,4-dimethyl-1-[(2S)-2-methylpiperazin-1-yl]pentan-1-one

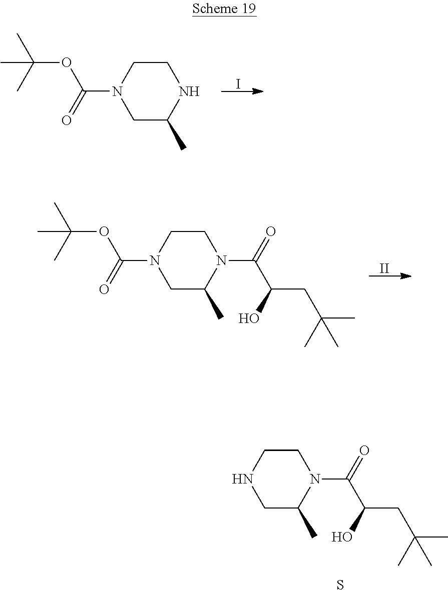

### Step I: (3S)-4-[(2R)-2-Hydroxy-4,4-dimethyl-pentanoyl]-3-methyl-piperazine-1-carboxylate

To the solution of (2R)-2-hydroxy-4,4-dimethyl-pentanoic acid (0.40 g, 2.74 mmol) in DMF (10 mL) were added tert-butyl (3S)-3-methylpiperazine-1-carboxylate (712 mg, 3.56 mmol), HATU (1.35 g, 3.56 mmol) and Hünig's base (950 μL, 5.47 mmol). The solution was stirred at room temperature overnight. The solution was diluted with EtOAc and the organic phase washed with water and brine, dried over Na₂SO₄ and filtered. The filtrate was concentrated under reduced pressure. The residue was purified by flash chromatography on silica gel eluting with 0-40% EtOAc: Hexanes affording the title compound. tert-Butyl (3S)-4-[(2R)-2-hydroxy-4,4-dimethyl-pentanoyl]-3-methyl-piperazine-1-carboxylate (620 mg, 69% yield). ¹H NMR (400 MHz, Chloroform-d) δ 4.86-3.71 (m, 4H), 3.60 (dd, J=31.5, 7.9 Hz, 1H), 3.43-3.18 (m, 1H), 3.13-2.55 (m, 2H), 1.46 (s, 9H), 1.39-1.07 (m, 5H), 1.05-0.93 (m, 9H).

### Step II: (2R)-2-Hydroxy-4,4-dimethyl-1-[(2S)-2-methylpiperazin-1-yl]pentan-1-one (Intermediate S)

To a solution of tert-butyl (3S)-4-[(2R)-2-hydroxy-4,4-dimethyl-pentanoyl]-3-methyl-piperazine-1-carboxylate (620 mg, 1.89 mmol) in DCM (2 mL) was added TFA (1.00 mL, 12.98 mmol). The solution was stirred at room temperature for 2 h. The reaction mixture was neutralized with a saturated aq. NaHCO₃ solution, diluted with DCM and the phases are separated. The aqueous phase extracted twice with DCM and the combined organic phase was washed with brine, dried over Na₂SO₄ and filtered. The filtrate was concentrated under reduced pressure affording the title compound which was used directly in the subsequent step without further purification. (2R)-2-Hydroxy-4,4-dimethyl-1-[(2S)-2-methylpiperazin-1-yl]pentan-1-one (310 mg, 72% yield). ESI-MS m/z calc. 228.18378, found 229.66 (M+1)⁺; Retention time: 0.86 minutes using method A.

### Preparation of Intermediate T: [7-tert-Butyl-5-(4-fluoro-phenyl)furo[3,2-b]pyridin-2-yl]-(2,2-dimethylpiperazin-1-yl)methanone hydrochloride

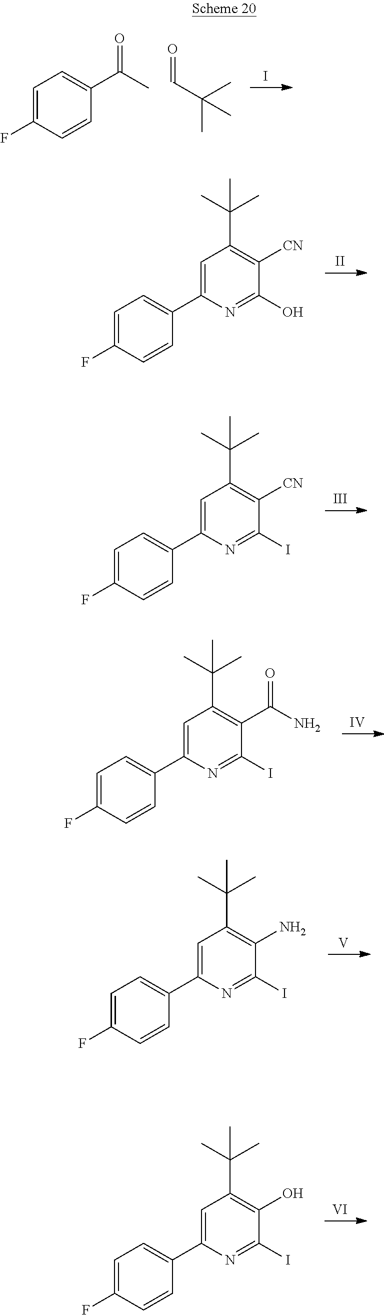

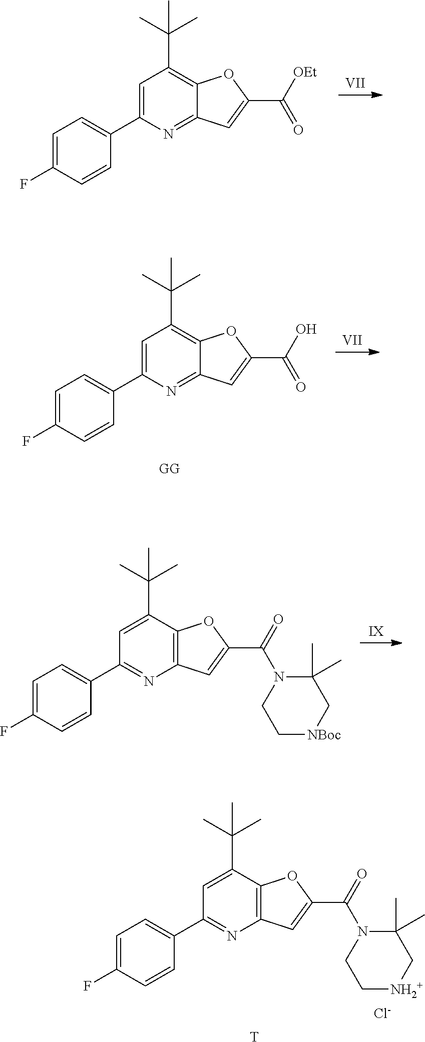

### Step I: 4-tert-Butyl-6-(4-fluorophenyl)-2-hydroxy-pyridine-3-carbonitrile

The intermediate was prepared according to General Procedure 4 using 1-(4-fluorophenyl)ethanone (19.84 g, 143.6 mmol), 2,2-dimethylpropanal (12 mL, 110.5 mmol), ethyl 2-cyanoacetate (11.78 mL, 110.5 mmol), ethanol (110 mL) and ammonium acetate (80.94 g, 1.05 mol). Then DCM (130 mL) and DDQ (20.07 g, 88.40 mmol) were added affording the title compound. 4-(tert-Butyl)-6-(4-fluorophenyl)-2-hydroxynicotinonitrile (14.8 g, 50% yield). ESI-MS m/z calc. 270.11685, found 271.27 (M+1)+; Retention time: 1.43 minutes using method C.

### Step II: 4-tert-Butyl-6-(4-fluorophenyl)-2-iodo-pyridine-3-carbonitrile

The intermediate was prepared according to General Procedure 5 using 4-(tert-butyl)-6-(4-fluorophenyl)-2-hydroxynicotinonitrile (10.00 g, 37.00 mmol) and pyridine (3.89 mL, 48.10 mmol) in acetonitrile (37 mL) at 0° C. and using triflic anhydride (7.47 mL, 44.40 mmol). Then NaI (27.73 g, 185.0 mmol) and HCl (4.63 mL of 12 M, 55.50 mmol) were added affording the title compound. 4-tert-Butyl-6-(4-fluorophenyl)-2-iodo-pyridine-3-carbonitrile (9.5 g, 67% yield). ESI-MS m/z calc. 380.0186, found 381.15 (M+1)⁺; Retention time: 2.19 minutes using method C.

### Step III: 4-tert-Butyl-6-(4-fluorophenyl)-2-iodo-pyridine-3-carboxamide

The intermediate was prepared according to General Procedure 7 using 4-(tert-butyl)-6-(4-fluorophenyl)-2-iodonicotinonitrile (800 mg, 2.10 mmol) and sulfuric acid (1.12 mL, 21.0 mmol) affording the title compound. 4-tert-Butyl-6-(4-fluorophenyl)-2-iodo-pyridine-3-carboxamide (843 mg, 100% yield). ESI-MS m/z calc. 398.02914, found 399.16 (M+1)⁺; Retention time: 1.57 minutes using method C.

### Step IV: 4-tert-Butyl-6-(4-fluorophenyl)-2-iodo-pyridin-3-amine

The intermediate was prepared according to General Procedure 8 using 4-tert-butyl-6-(4-fluorophenyl)-2-iodo-pyridine-3-carboxamide (20.00 g, 50.22 mmol), KOH (19.72 g, 351.5 mmol), water (55 mL) and bromine (3.36 mL, 65.29 mmol), then water (19 mL) and THF (19 mL) affording the title compound. 4-tert-Butyl-6-(4-fluorophenyl)-2-iodo-pyridin-3-amine (17.21 g, 93% yield). ESI-MS m/z calc. 370.0342. found 371.17 (M+1)⁺; Retention time: 2.13 minutes using method C.

### Step V: 4-tert-Butyl-6-(4-fluorophenyl)-2-iodo-pyridin-3-ol

The intermediate was prepared according to General Procedure 9 using 4-tert-butyl-6-(4-fluorophenyl)-2-iodo-pyridin-3-amine (4.99 g, 13.5 mmol) in TFA (24 mL) and isopentyl nitrite (2.72 mL, 20.2 mmol) affording the title compound. 4-tert-Butyl-6-(4-fluorophenyl)-2-iodo-pyridin-3-ol (5.1 g, 100% yield). ESI-MS m/z calc. 371.0182, found 372.29 (M+1)⁺; Retention time: 2.14 minutes using method C.

### Step VI: Ethyl 7-tert-butyl-5-(4-fluorophenyl)furo[3,2-b]pyridine-2-carboxylate

The intermediate was prepared according to General Procedure 10 using 4-tert-butyl-6-(4-fluorophenyl)-2-iodo-pyridin-3-ol (5.1 g, 13.7 mmol) in THF (26 mL), potassium carbonate (13.29 g, 96.2 mmol), PdCl₂(PPh₃)₂ (386 mg, 0.55 mmol) and CuI (209 mg, 1.10 mmol). Then ethyl prop-2-ynoate (5.57 mL, 55.0 mmol) in THF (13 mL) was added affording the title compound. Ethyl 7-tert-butyl-5-(4-fluorophenyl)furo[3,2-b]pyridine-2-carboxylate (348 mg, 62% yield). ESI-MS m/z calc. 341.1427, found 342.61 (M+1)⁺; Retention time: 2.21 minutes using method C.

### Step VII: 7-tert-Butyl-5-(4-fluorophenyl)furo[3,2-b]pyridine-2-carboxylic acid Intermediate GG)

The intermediate was prepared according to General Procedure 11 using ethyl 7-tert-butyl-5-(4-fluorophenyl)furo[3,2-b]pyridine-2-carboxylate (970 mg, 2.84 mmol) and aqueous LiOH (2.84 mL of 2 M, 5.68 mmol) in dioxane (15 mL) affording the title compound. 7-tert-Butyl-5-(4-fluorophenyl)furo[3,2-b]pyridine-2-carboxylic acid (867 mg, 97% yield). ¹H NMR (400 MHz, DMSO-d6) δ 8.23-8.13 (m, 2H), 7.83-7.77 (m, 2H), 7.39-7.28 (m, 2H), 1.54 (s, 9H). ESI-MS m/z calc. 313.1114, found 314.52 (M+1)⁺; Retention time 1.64 minutes using method C.

### Step VIII: tert-Butyl 4-[7-tert-butyl-5-(4-fluorophenyl)furo[3,2-b]pyridine-2-carbonyl]-3,3-dimethyl-piperazine-1-carboxylate

The intermediate was prepared according to General Procedure 1 using 7-tert-butyl-5-(4-fluorophenyl)furo[3,2-b]pyridine-2-carboxylic acid (620 mg, 1.98 mmol), DMF (10 mL), HATU (941 mg, 2.47 mmol), tert-butyl 3,3-dimethylpiperazine-1-carboxylate (488 mg, 2.28 mmol) and Hünig's base (1.21 mL, 6.93 mmol) affording the title compound. tert-Butyl 4-[7-tert-butyl-5-(4-fluorophenyl)furo[3,2-b]pyridine-2-carbonyl]-3,3-dimethyl-piperazine-1-carboxylate (1.0 g, 99% yield). ESI-MS m/z calc. 509.2690, found 510.51 (M+1)⁺; Retention time: 2.25 minutes using method C.

### Step IX: [7-tert-Butyl-5-(4-fluorophenyl)furo[3,2-b]pyridin-2-yl]-(2,2-dimethylpiperazin-1-yl)methanone hydrochloride (Intermediate T)

The intermediate was prepared according to General Procedure 2 using tert-butyl 4-[7-tert-butyl-5-(4-fluorophenyl)furo[3,2-b]pyridine-2-carbonyl]-3,3-dimethyl-piperazine-1-carboxylate (439 mg, 0.8614 mmol). HCl solution in 1,4-dioxane (4.31 mL of 4 M, 17.23 mmol) and DCM (9.4 mL) affording the title compound. [7-tert-Butyl-5-(4-fluorophenyl)furo[3,2-b]pyridin-2-yl]-(2,2-dimethylpiperazin-1-yl)methanone hydrochloride (384 mg, 100% yield). ESI-MS m/z calc. 409.2166, found 410.36 (M+1)*; Retention time: 1.48 minutes using method C.

### Preparation of Intermediate U: [7-tert-Butyl-5-(4-(trifluoromethyl)phenyl)furo[3,2-b]pyridin-2-yl]-(2,2-dimethylpiperazin-1-yl)methanone hydrochloride

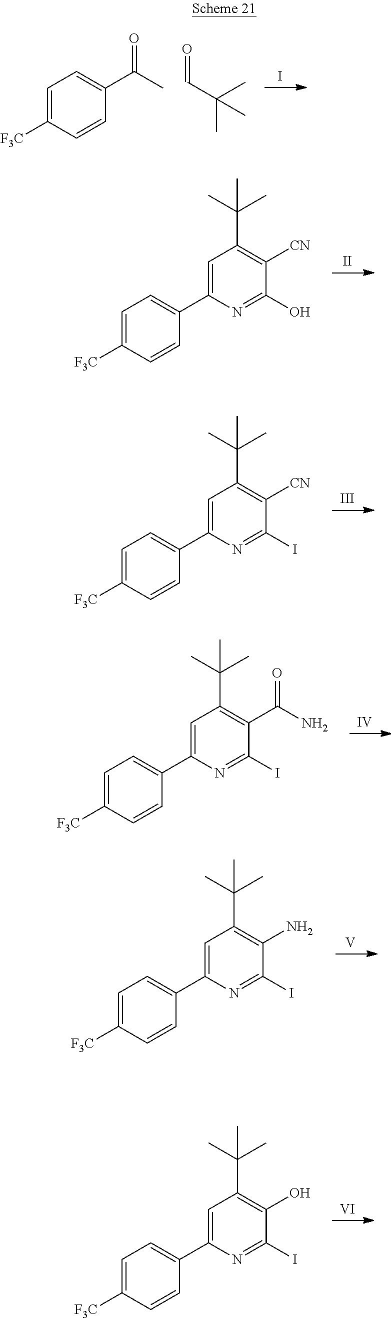

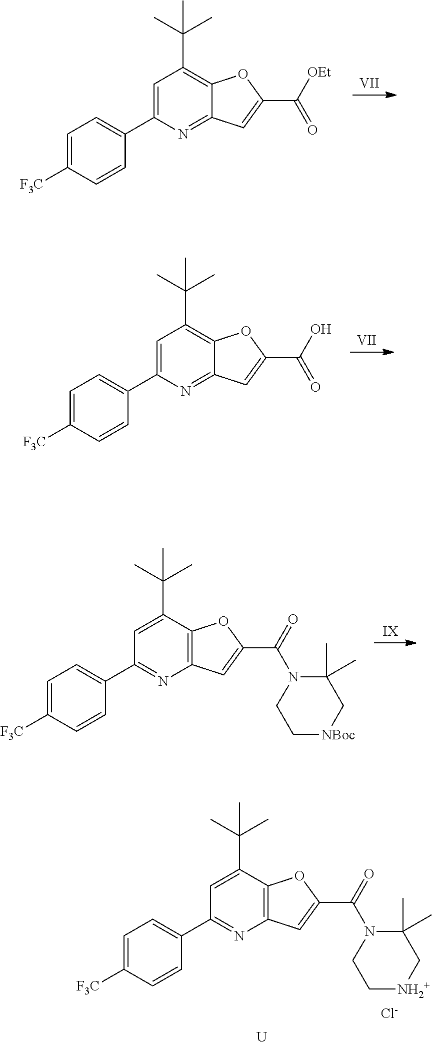

### Step I: 4-tert-Butyl-2-hydroxy-6-[4-(trifluoromethyl)phenyl]pyridine-3-carbonitrile

The intermediate was prepared according to General Procedure 4 using 1-[4-(trifluoromethyl)phenyl]ethanone (30.27 g, 160.9 mmol). 2,2-dimethylpropanal (10.0 g. 116.1 mmol), ethyl 2-cyanoacetate (12.61 mL, 118.3 mmol), ethanol (126.1 mL) and ammonium acetate (88.26 g, 1.145 mol) then DCM (462 mL) and DDQ (20.96 g, 92.32 mmol) were added affording the title compound. 4-tert-Butyl-2-hydroxy-6-[4-(trifluoromethyl)phenyl]pyridine-3-carbonitrile (14.00 g, 38% yield). ESI-MS m/z calc. 320.11365, found 321.30 (M+1)+; Retention time: 1.80 minutes using method C.

### Step II: 4-tert-Butyl-2-iodo-6-[4-(trifluoromethyl)phenyl]pyridine-3-carbonitrile

The intermediate was prepared according to General Procedure 5 using 4-tert-butyl-2-hydroxy-6-[4-(trifluoromethyl)phenyl]pyridine-3-carbonitrile (10.0 g, 31.22 mmol) and pyridine (2.88 mL, 35.65 mmol) in acetonitrile (62.44 mL) at 0° C. and triflic anhydride (5.77 mL, 34.28 mmol). Then NaI (23.40 g, 156.1 mmol) and triflic acid (3.04 mL, 34.34 mmol) were added affording the title compound. 4-tert-Butyl-2-iodo-6-[4-(trifluoromethyl)phenyl]pyridine-3-carbonitrile (6.00 g, 45% yield). ESI-MS m/z calc. 430.01538, found 431.21 (M+1)⁺; Retention time: 2.98 minutes using method B.

### Step III: 4-tert-Butyl-6-(4-fluorophenyl)-2-iodo-pyridine-3-carboxamide

To a round bottom flask containing 4-tert-butyl-2-iodo-6-[4-(trifluoromethyl)phenyl]pyridine-3-carbonitrile (2.6 g, 6.04 mmol) was added sulfuric acid (3.36 mL of 18 M, 60.44 mmol) and water (0.10 mL, 6.04 mmol) the suspension was stirred at 80° C. for 72 h. The solution was cooled to room temperature and added dropwise to a saturated solution of sodium carbonate in water. The resulting white precipitate was collected by filtration, washed with water and dried under reduced pressure, affording the title product as a white solid. 4-tert-Butyl-2-iodo-6-[4-(trifluoromethyl)phenyl]pyridine-3-carboxamide (2.30 g. 85% yield). ESI-MS m/z calc. 448.02594, found 449.28 (M+1)⁺; Retention time: 1.96 minutes using method C.

### Step IV: 4-tert-Butyl-2-iodo-6-[4-(trifluoromethyl)phenyl]pyridin-3-amine

The intermediate was prepared according to General Procedure 8 using 4-tert-butyl-2-iodo-6-[4-(trifluoromethyl)phenyl]pyridine-3-carboxamide (2.3 g, 5.13 mmol), KOH (2.02 g, 35.92 mmol), water (6.0 mL) and bromine (344 μL, 6.67 mmol), and then water (2.0 mL) and THF (2.0 mL) were added affording the title compound. 4-tert-Butyl-2-iodo-6-[4-(trifluoromethyl)phenyl]pyridin-3-amine (2.80 g, quantitative yield). ESI-MS m/z calc. 420.03103, found 421.27 (M+1)⁺; Retention time: 2.47 minutes using method C.

### Step V: 4-tert-Butyl-2-iodo-6-[4-(trifluoromethyl)phenyl]pyridin-3-ol

The intermediate was prepared according to General Procedure 9 using 4-tert-butyl-2-iodo-6-[4-(trifluoromethyl)phenyl]pyridin-3-amine (2.8 g, 6.66 mmol) in TFA (13.1 mL) and isopentyl nitrite (5.4 mL, 40.0 mmol) affording the title compound. 4-tert-Butyl-2-iodo-6-[4-(trifluoromethyl)phenyl]pyridin-3-ol (3.5 g, quantitative yield). ESI-MS m/z calc. 421.01504, found 422.48 (M+1)⁺; Retention time: 1.75 minutes using method I.

### Step VI: Ethyl 7-tert-butyl-5-[4-(trifluoromethyl)phenyl]furo[3,2-b]pyridine-2-carboxylate

The intermediate was prepared according to General Procedure 10 using 4-tert-butyl-2-iodo-6-[4-(trifluoromethyl)phenyl]pyridin-3-ol (2.80 g, 6.65 mmol), ethyl prop-2-ynoate (1.68 mL, 16.62 mmol) in THF (7.7 mL), potassium carbonate (4.59 g, 33.2 mmol), PdCl₂(PPh₃)₂ (187 mg, 0.27 mmol) and CuI (101 mg, 0.53 mmol) in THF (15.3 mL). Ethyl 7-tert-butyl-5-[4-(trifluoromethyl)phenyl]furo[3,2-b]pyridine-2-carboxylate. (1.0 g, 38% yield). ESI-MS m/z calc. 391.13953, found 392.65 (M+1)⁺; Retention time: 1.85 minutes using method I.

### Step VII: 7-tert-Butyl-5-[4-(trifluoromethyl)phenyl]furo[3,2-b]pyridine-2-carboxylic acid

The intermediate was prepared according to General Procedure 11 using ethyl 7-tert-butyl-5-[4-(trifluoromethyl)phenyl]furo[3,2-b]pyridine-2-carboxylate (1 g. 2.56 mmol) in dioxane (8.5 mL) and an aqueous solution of LiOH (2.56 mL of 2 M, 5.11 mmol) affording the title compound. 7-tert-Butyl-5-[4-(trifluoromethyl)phenyl]furo[3,2-b]pyridine-2-carboxylic acid (910 mg, 98% yield). ESI-MS m/z calc. 363.10823, found 344.54 (M+1)⁺; Retention time 2.08 minutes using method C.

### Step VIII: tert-Butyl 4-[7-tert-butyl-5-[4-(trifluoromethyl)phenyl]furo[3,2-b]pyridine-2-carbonyl]-3,3-dimethyl-piperazine-1-carboxylate

The intermediate was prepared according to General Procedure 1 using 7-tert-butyl-5-[4-(trifluoromethyl)phenyl]furo[3,2-b]pyridine-2-carboxylic acid (1.0 g, 2.75 mmol), DMF (5.5 mL), HATU (1.36 g, 3.58 mmol), tert-butyl 3,3-dimethylpiperazine-1-carboxylate (590 mg, 2.75 mmol) and Hünig's base (1.68 mL, 9.63 mmol) affording the title compound. tert-Butyl 4-[7-tert-butyl-5-[4-(trifluoromethyl)phenyl]furo[3,2-b]pyridine-2-carbonyl]-3,3-dimethyl-piperazine-1-carboxylate (1.4 g, 91% yield). ESI-MS m/z calc. 559.26579, found 560.22 (M+1)⁺; Retention time: 1.87 minutes using method 1.

### Step IX: [7-tert-Butyl-5-[4-(trifluoromethyl)phenyl]furo[3,2-b]pyridin-2-yl]-(2,2-dimethylpiperazin-1-yl)methanone hydrochloride (Intermediate U)

The intermediate was prepared according to General Procedure 2 using tert-butyl 4-[7-tert-butyl-5-[4-(trifluoromethyl)phenyl]furo[3,2-b]pyridine-2-carbonyl]-3,3-dimethyl-piperazine-1-carboxylate (1.4 g, 2.50 mmol), 4N HCl solution in 1,4-dioxane (6.26 mL, 25.0 mmol) and dioxane (17.9 mL) affording the title compound. [7-tert-Butyl-5-[4-(trifluoromethyl)phenyl]furo[3,2-b]pyridin-2-yl]-(2,2-dimethylpiperazin-1-yl)methanone hydrochloride (1.0 g, 85% yield). ESI-MS m/z calc. 459.21336, found 460.49 (M+1)⁺; Retention time: 1.58 minutes using method C.

### Preparation of Intermediate V: 7-(tert-butyl)-5-(4-chlorophenyl)furo[3,2-b]pyridine-2-carboxylic acid

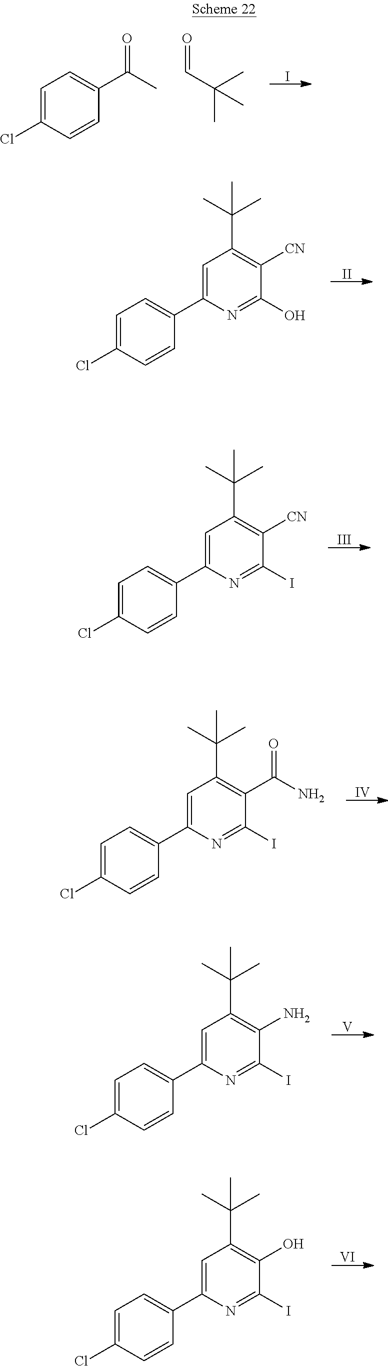

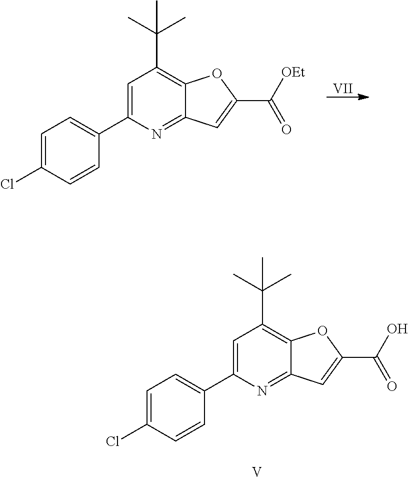

### Step I: 4-tert-Butyl-6-(4-chlorophenyl)-2-hydroxy-pyridine-3-carbonitrile

Using 1-(4-chlorophenyl)ethanone (18.50 g, 15.5 mL, 119.7 mmol) as the starting material and following General Procedure 4, the title compound was obtained as a pink solid (9.09 g, 34% yield) after a final trituration in hot EtOH. ¹H NMR (400 MHz, Chloroform-d) δ 7.88-7.78 (m, 2H), 7.61-7.54 (m, 2H), 6.70 (s, 1H), 1.55 (s, 9H). ESI-MS m/z calc. 286.08728, found 287.35 (M+1)⁺, Retention time: 1.72 minutes using method C.

### Step II: 4-tert-Butyl-6-(4-chlorophenyl)-2-iodo-pyridine-3-carbonitrile

Using 4-tert-butyl-6-(4-chlorophenyl)-2-hydroxy-pyridine-3-carbonitrile (9.07 g, 31.63 mmol) as the starting material and following General Procedure 5 using triflic acid (3.36 mL, 38.0 mmol) in the second step, the crude product was obtained. The residue was purified by flash chromatography on silica gel eluting with 0-80% DCM: Hexanes. The title compound (6.58 g, 52% yield) was obtained as a light yellow solid. ¹H NMR (400) MHz, CDCl₃) δ 8.00-7.90 (m, 2H), 7.72 (s, 1H), 7.52-7.41 (m, 2H), 1.56 (s, 9H). ESI-MS m/z calc. 395.98902, found 397.22 (M+1)⁺; Retention time: 2.93 minutes using method B.

### Step III: 4-tert-Butyl-6-(4-chlorophenyl)-2-iodo-pyridine-3-carboxamide

Using 4-tert-butyl-6-(4-chlorophenyl)-2-iodo-pyridine-3-carbonitrile (6.60 g, 16.64 mmol) as the starting material and following General Procedure 7, the reaction mixture was neutralized using potassium carbonate in water (42.00 g in 250 mL). The resulting white precipitate is extracted with CHCl₃/iPrOH mixture (4:1, 3×100 mL). The combined organic extracts were washed with H₂O (100 mL), brine (100 mL), dried over MgSO₄, filtered and concentrated, affording the title compound (6.97 g, 100% yield) as an off-white solid. ¹H NMR (400 MHz, CDCl₃) δ 7.94-7.83 (m, 2H), 7.70 (s, 1H), 7.49-7.39 (m, 2H), 6.03 (broad s, 1H), 5.80 (broad s, 1H), 1.49 (s, 9H). ESI-MS m/z calc. 413.99957, found 415.25 (M+1)⁺; Retention time: 1.9 minutes using method C.

### Step IV: 4-tert-Butyl-6-(4-chlorophenyl)-2-iodo-pyridin-3-amine

Using 4-tert-butyl-6-(4-chlorophenyl)-2-iodo-pyridine-3-carboxamide (3.03 g. 7.31 mmol) as the starting material and following General Procedure 8, the title compound (2.66 g, 94% yield) was obtained as a dark purple solid which was used in the next step without purification. ¹H NMR (400 MHz. CDCl₃) δ 7.84-7.77 (m, 2H), 7.45 (s, 1H), 7.39-7.34 (m, 2H), 4.45 (s, 2H), 1.45 (s, 9H). ESI-MS m/z calc. 386.00467, found 387.23 (M+1)⁺; Retention time: 2.46 minutes using method C.

### Step V: 4-tert-Butyl-6-(4-chlorophenyl)-2-iodo-pyridin-3-ol

Using 4-tert-butyl-6-(4-chlorophenyl)-2-iodo-pyridin-3-amine (2.99 g, 7.73 mmol) as the starting material and following General Procedure 9, the reaction mixture after the hydrolysis step was diluted with EtOAc and H₂O (150 mL each) and the layers are separated. The aqueous layer was extracted with EtOAc (2×75 mL). The combined organic extracts were washed with brine (150 mL), dried over MgSO₄, filtered and concentrated. The residue was purified by flash chromatography on silica gel eluting with 0-30% DCM: Hexanes. The title compound (2.35 g, 78% yield) was obtained as a white solid. ¹H NMR (400 MHz, CDCl₃) δ 7.85-7.79 (m, 2H), 7.48 (s, 1H), 7.43-7.36 (m, 2H), 5.60 (broad s, 1H), 1.43 (s, 9H). ESI-MS m/z calc. 386.98868, found 388.16 (M+1)⁺; Retention time: 1.74 minutes using method I.

### Step VI: Ethyl 7-tert-butyl-5-(4-chlorophenyl)furo[3,2-b]pyridine-2-carboxylate

Using 4-tert-butyl-6-(4-chlorophenyl)-2-iodo-pyridin-3-ol (3.13 g, 8.07 mmol) as the starting material and following General Procedure 10, the reaction mixture was poured carefully into a flask containing 1N HCl (120 mL) and EtOAc (100 mL). The layers were separated and the aqueous layer was extracted with EtOAc (100 mL, 2×50 mL). The combined organic extracts were washed with half-saturated brine (100 mL), dried over MgSO₄ and filtered through Celite®, rinsing with EtOAc and concentrated. The residue was purified by flash chromatography on silica gel eluting with 0-10% EtOAc: Hexanes. The title compound (1.08 g, 37% yield) was obtained as a pale yellow solid. ¹H NMR (400 MHz, CDCl₃) δ 8.00-7.86 (m, 2H), 7.68 (s, 1H), 7.62 (s, 1H), 7.53-7.39 (m, 2H), 4.47 (q, J=7.1 Hz, 2H), 1.58 (s, 9H), 1.45 (t, J=7.1 Hz, 3H). ESI-MS m/z calc. 357.11316, found 358.61 (M+1)⁺; Retention time: 1.84 minutes using method I.

### Step VII: 7-tert-Butyl-5-(4-chlorophenyl)furo[3,2-b]pyridine-2-carboxylic acid (Intermediate V)

Using ethyl 7-tert-butyl-5-(4-chlorophenyl)furo[3,2-b]pyridine-2-carboxylate (1.06 g, 2.96 mmol) as the starting material and following General Procedure 11, the reaction mixture was neutralized with 1N HCl (6 mL) and extracted with DCM (20 mL, 2×10 mL). The combined organic extracts washed with brine (10 mL), and the brine layer was further extracted with DCM (2×10 mL). The combined organic extracts were dried over MgSO₄, filtered and concentrated, affording the title compound (1.14 g, 100% yield) as a pale yellow solid. ¹H NMR (400 MHz, CDCl₃ and DMSO-d6) δ 7.81-7.67 (m, 2H), 7.44 (s, 1H), 7.42 (s, 1H), 7.32-7.20 (m, 2H), 1.38 (s, 9H). ESI-MS m/z calc. 329.08188. found 330.31 (M+1)⁺; Retention time: 1.07 minutes using method I.

### Preparation of Intermediate W: [7-tert-Butyl-5-(4-fluoro-3-methyl-phenyl)furo[3,2-b]pyridin-2-yl]-(2,2-dimethylpiperazin-1-yl)methanone hydrochloride

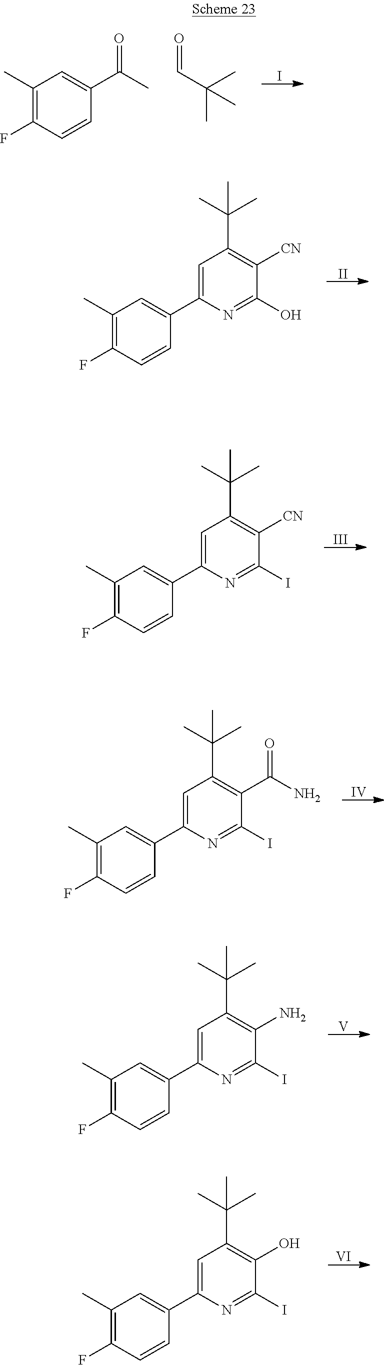

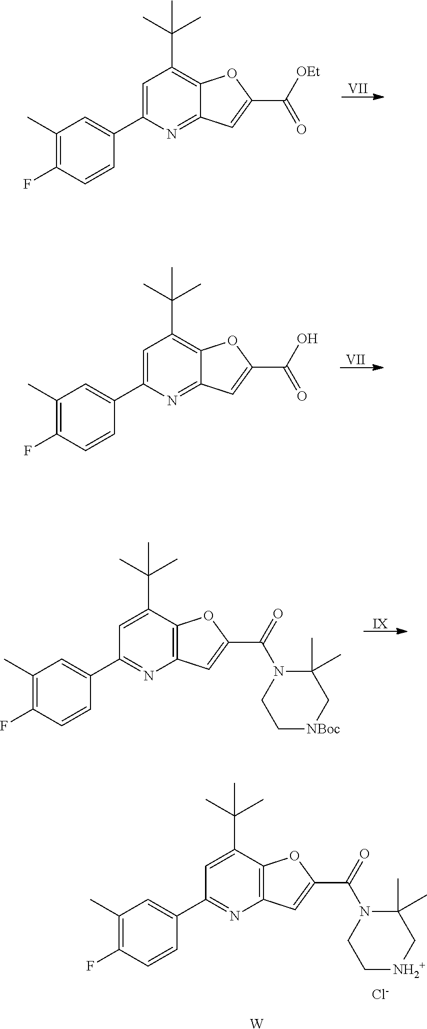

### Step I: 4-(tert-Butyl)-6-(4-fluoro-3-methylphenyl)-2-hydroxynicotinonitrile

The intermediate was prepared according to General Procedure 4 using 1-(4-fluoro-3-methyl-phenyl)ethanone (10.00 g, 63.8 mmol), 2,2-dimethylpropanal (5.32 mL, 49.0 mmol), ethyl 2-cyanoacetate (5.32 mL, 49.9 mmol), ethanol (53 mL) and ammonium acetate (37.3 g, 483.4 mmol). Then DCM (105 mL) and DDQ (8.34 g, 36.8 mmol) were added affording the title compound. 4-(tert-Butyl)-6-(4-fluoro-3-methylphenyl)-2-hydroxynicotinonitrile (6.22 g, 45% yield). ¹H NMR (400 MHz, DMSO-d6) δ 12.66 (s, 1H), 7.82 (dd, J=7.5, 2.6 Hz, 1H), 7.72 (ddd, J=7.9, 4.8, 2.4 Hz, 1H), 7.30 (dd, J=9.6, 8.6 Hz, 1H), 6.70 (s, 1H), 2.30 (d, J=2.0 Hz, 3H), 1.45 (s, 9H). ESI-MS m/z calc. 284.1325, found 285.3 (M+1)⁺; Retention time: 1.57 minutes using method C.

### Step II: 4-(tert-Butyl)-6-(4-fluoro-3-methylphenyl)-2-iodonicotinonitrile

The intermediate was prepared according to General Procedure 5 using 4-(tert-butyl)-6-(4-fluoro-3-methylphenyl)-2-hydroxynicotinonitrile (6.22 g, 21.9 mmol) and pyridine (2.04 mL, 25.2 mmol) in acetonitrile (37 mL) at 0° C. and triflic anhydride (4.05 mL, 24.1 mmol). Then NaI (16.4 g. 109.4 mmol) and HCl (2 mL of 12N, 24.1 mmol) were added affording the title compound. 4-(tert-Butyl)-6-(4-fluoro-3-methylphenyl)-2-iodonicotinonitrile (4.11 g, 48% yield). ESI-MS m/z calc. 394.0342 found 395.2 (M+1)⁺; Retention time: 1.51 minutes using method I.

### Step III: 4-tert-Butyl-6-(4-fluoro-3-methyl-phenyl)-2-iodo-pyridine-3-carboxamide

The intermediate was prepared according to General Procedure 7 using 4-(tert-butyl)-6-(4-fluoro-3-methylphenyl)-2-iodonicotinonitrile (4.65 g, 11.8 mmol) and sulfuric acid (9.43 mL, 177 mmol) affording the title compound. 4-tert-Butyl-6-(4-fluoro-3-methyl-phenyl)-2-iodo-pyridine-3-carboxamide (4.75 g. 98% yield). ESI-MS m/z calc. 412.0448, found 413.2 (M+1)⁺; Retention time: 0.68 minutes using method I.

### Step IV: 4-tert-Butyl-6-(4-fluoro-3-methyl-phenyl)-2-iodo-pyridin-3-amine

The intermediate was prepared according to General Procedure 8 using 4-tert-butyl-6-(4-fluoro-3-methyl-phenyl)-2-iodo-pyridine-3-carboxamide (4.75 g, 11.5 mmol), KOH (4.49 g, 80.0 mmol), water (13.2 mL) and bromine (772 μL, 15 mmol), then water (4.4 mL) and THF (4.4 mL) affording the title compound. 4-tert-Butyl-6-(4-fluoro-3-methyl-phenyl)-2-iodo-pyridin-3-amine (2.68 g, 60% yield). ESI-MS m/z calc. 384.0499, found 385.2 (M+1)⁺; Retention time: 1.40 minutes using method I.

### Step V: 4-tert-Butyl-6-(4-fluoro-3-methyl-phenyl)-2-iodo-pyridin-3-ol

The intermediate was prepared according to General Procedure 9 using 4-tert-butyl-6-(4-fluoro-3-methyl-phenyl)-2-iodo-pyridin-3-amine (2.68 g, 6.98 mmol) in TFA (12.5 mL) and isopentyl nitrite (5.6 mL, 41.9 mmol) affording the title compound. 4-tert-Butyl-6-(4-fluoro-3-methyl-phenyl)-2-iodo-pyridin-3-ol (2.29 g, 85% yield). ESI-MS m/z calc. 385.0339, found 386.2 (M+1)⁺; Retention time: 1.44 minutes using method I.

### Step VI: Ethyl 7-tert-butyl-5-(4-fluoro-3-methyl-phenyl)furo[3,2-b]pyridine-2-carboxylate

The intermediate was prepared according to General Procedure 10 using 4-tert-butyl-6-(4-fluoro-3-methyl-phenyl)-2-iodo-pyridin-3-ol (2.29 g, 5.95 mmol) in THF (12 mL), potassium carbonate (5.75 g, 41.6 mmol), PdCl₂(PPh₃)₂ (167 mg, 0.24 mmol) and CuI (91 mg, 0.48 mmol). Then ethyl prop-2-ynoate (2.4 mL, 23.8 mmol) in THF (6 mL) was added affording the title compound. Ethyl 7-tert-butyl-5-(4-fluoro-3-methyl-phenyl)furo[3,2-b]pyridine-2-carboxylate (610 mg, 29% yield). ESI-MS m/z calc. 355.1584, found 357.3 (M+1)⁺; Retention time: 1.53 minutes using method I.

### Step VII: 7-tert-Butyl-5-(4-fluoro-3-methyl-phenyl)furo[3,2-b]pyridine-2-carboxylic acid

The intermediate was prepared according to General Procedure 11 using ethyl 7-tert-butyl-5-(4-fluoro-3-methyl-phenyl)furo[3,2-b]pyridine-2-carboxylate (610 mg, 1.72 mmol) in dioxane (9.4 mL, 0.2M) and LiOH (1.72 mL of 2M, 3.43 mmol) affording the title compound. 7-tert-Butyl-5-(4-fluoro-3-methyl-phenyl)furo[3,2-b]pyridine-2-carboxylic acid (520 mg, 92% yield). ¹H NMR (400 MHz, DMSO-d6) δ 8.06 (ddd, J=7.6, 2.4, 1.0 Hz 1H), 7.97 (ddd, J=8.4, 4.0, 1.5 Hz, 1H), 7.76 (d, J=11.3 Hz, 2H), 7.26 (dd, J=9.6, 8.6 Hz, 1H), 2.34 (d, J=1.9 Hz, 3H), 1.54 (s, 9H). ESI-MS m/z calc. 327.1271, found 328.3 (M+1)⁺; Retention time: 3.41 minutes using method A.

### Step VIII: tert-Butyl 4-[7-tert-butyl-5-(4-fluoro-3-methyl-phenyl)furo[3,2-b]pyridine-2-carbonyl]-3,3-dimethyl-piperazine-1-carboxylate

The intermediate was prepared according to General Procedure 1 using 7-tert-butyl-5-(4-fluoro-3-methyl-phenyl)furo[3,2-b]pyridine-2-carboxylic acid (100 mg, 0.31 mmol), DMF (1 mL, 0.3M), HATU (141 mg, 0.37 mmol), tert-butyl 3,3-dimethylpiperazine-1-carboxylate (73 mg, 0.34 mmol) and Hünig's base (187 μL, 1.07 mmol) affording the title compound. tert-Butyl 4-[7-tert-butyl-5-(4-fluoro-3-methyl-phenyl)furo[3,2-b]pyridine-2-carbonyl]-3,3-dimethyl-piperazine-1-carboxylate (160 mg, 100% yield). ESI-MS m/z calc. 523.2846, found 525.5 (M+1)⁺; Retention time: 1.56 minutes using method 1.

### Step IX: [7-tert-Butyl-5-(4-fluoro-3-methyl-phenyl)furo[3,2-b]pyridin-2-yl]-(2,2-dimethylpiperazin-1-yl)methanone hydrochloride (Intermediate W)

The intermediate was prepared according to General Procedure 2 using tert-butyl 4-[7-tert-butyl-5-(4-fluoro-3-methyl-phenyl)furo[3,2-b]pyridine-2-carbonyl]-3,3-dimethyl-piperazine-1-carboxylate (155 mg, 0.30 mmol) and 4N HCl solution in 1,4-dioxane (2.2 mL, 8.8 mmol) affording the title compound. [7-tert-Butyl-5-(4-fluoro-3-methyl-phenyl)furo[3,2-b]pyridin-2-yl]-(2,2-dimethylpiperazin-1-yl)methanone hydrochloride (147 mg, 100% yield). ¹H NMR (400 MHz, DMSO-d6) δ 8.19 (s, 1H), 8.05 (ddd, J=7.6, 2.3, 0.9 Hz, 1H), 7.96 (ddd, J=7.9, 5.0, 2.4 Hz, 1H), 7.73 (s, 1H), 7.46 (s, 1H), 7.29-7.20 (m, 1H), 3.49-3.44 (m, 2H), 2.87 (dd, J=5.9, 4.4 Hz, 2H), 2.65 (s, 2H), 2.34 (d, J=1.9 Hz, 3H), 1.52 (s, 9H), 1.44 (s, 6H). ESI-MS m/z calc. 423.23221, found 424.41 (M+1)⁺; Retention time: 2.87 minutes using method A.

### Preparation of Intermediate X: [7-tert-Butyl-5-(p-tolyl)furo[3,2-b]pyridin-2-yl]-(2,2-dimethylpiperazin-1-yl)methanone hydrochloride

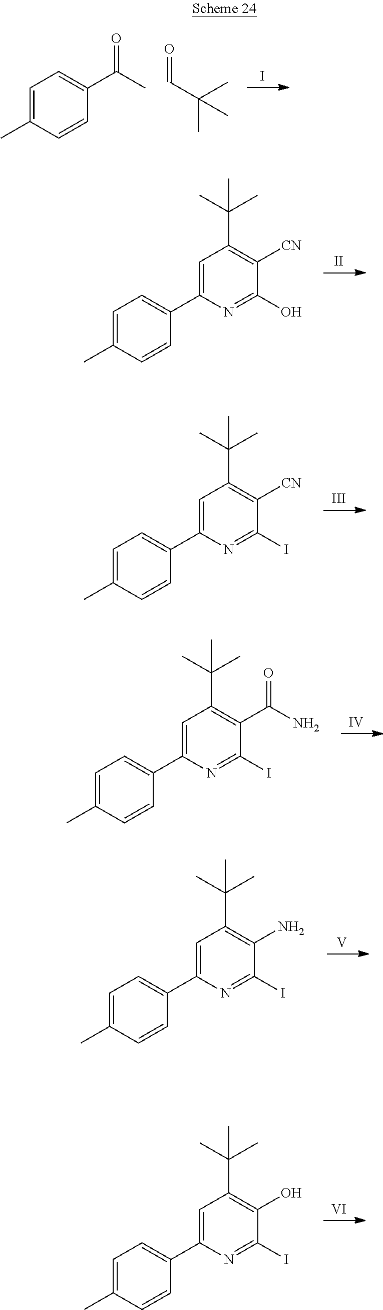

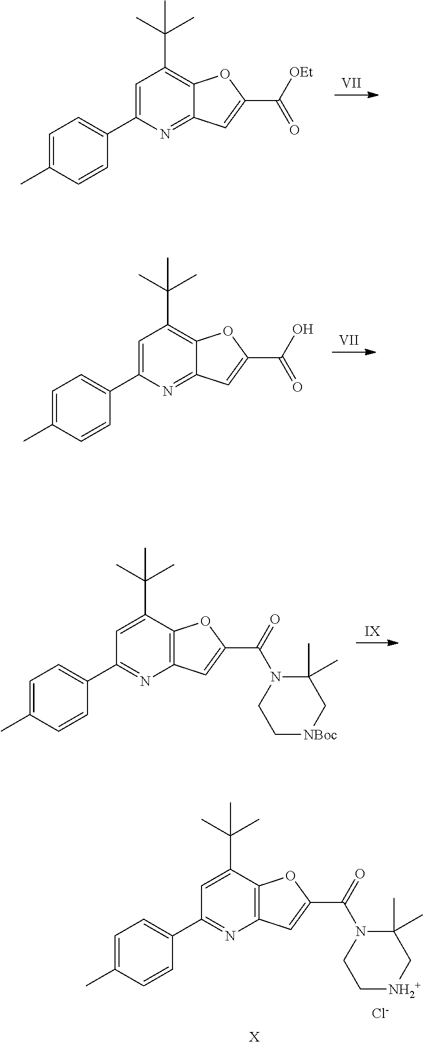

### Step I: 4-(tert-Butyl)-6-(p-tolyl)-2-hydroxynicotinonitrile

The intermediate was prepared according to General Procedure 4 using 1-(4-methyl-phenyl)ethanone (24.0 mL, 179.6 mmol), 2,2-dimethylpropanal (15.0 mL, 138.1 mmol), ethyl 2-cyanoacetate (15.0 mL, 140.7 mmol), ethanol (150 mL) and ammonium acetate (105 g, 1.36 mol). Then DCM (300 mL) and DDQ (23.52 g, 103.6 mmol) were added affording the title compound. 4-(tert-Butyl)-6-(p-tolyl)-2-hydroxynicotinonitrile (17.6 g, 48% yield). ¹H NMR (400 MHz, DMSO-d6) δ 12.68 (s, 1H), 7.74 (d, J=7.8 Hz, 2H), 7.40-7.28 (m, 2H), 6.66 (s, 1H), 2.37 (s, 3H), 1.45 (s, 9H). ESI-MS m/z calc. 266.1419, found 267.3 (M+1)⁺; Retention time: 1.52 minutes using method C.

### Step II: tert-Butyl 4-tert-butyl-2-iodo-6-(4-methylphenyl)pyridine-3-carbonitrile

The intermediate was prepared according to General Procedure 6 using 4-(tert-butyl)-6-(4-methylphenyl)-2-hydroxynicotinonitrile (3 g, 11.26 mmol) and pyridine (1.05 mL, 12.95 mmol) in acetonitrile (18 mL) at 0° C. and triflic anhydride (2.09 mL, 12.39 mmol). Evaporated to a residue after 30 minutes. Then NaI (16.88 g, 112.6 mmol), NMP (18 mL) and HCl (1.03 mL of 12N, 12.39 mmol) were added affording the title compound. 4-tert-Butyl-2-iodo-6-(4-methylphenyl)pyridine-3-carbonitrile (2.0 g, 47% yield). ESI-MS m/z calc. 376.0436, found 377.2 (M+1)⁺; Retention time: 1.51 minutes using method I.

### Step III: 4-tert-Butyl-6-(4-methylphenyl)-2-iodo-pyridine-3-carboxamide

The intermediate was prepared according to General Procedure 7 using 4-(tert-butyl)-6-(4-methylphenyl)-2-iodonicotinonitrile (2.00 g, 5.32 mmol) and sulfuric acid (4.25 mL, 79.74 mmol) affording the title compound. 4-tert-Butyl-6-(4-methylphenyl)-2-iodo-pyridine-3-carboxamide (1.87 g, 89% yield). ESI-MS m/z calc. 394.0542, found 395.3 (M+1)⁺; Retention time: 0.65 minutes using method I.

### Step IV: 4-tert-Butyl-6-(4-methylphenyl)-2-iodo-pyridin-3-amine

The intermediate was prepared according to General Procedure 8 using 4-tert-butyl-6-(4-methylphenyl)-2-iodo-pyridine-3-carboxamide (1.75 g, 4.44 mmol), KOH (1.73 g, 30.83 mmol), water (4.85 mL) and bromine (297 μL, 5.77 mmol) then water (1.61 mL) and THF (1.61 mL) affording the title compound. 4-tert-Butyl-6-(4-methylphenyl)-2-iodo-pyridin-3-amine (1.20 g, 74% yield). ESI-MS m/z calc. 366.0593, found 368.2 (M+1)⁺; Retention time: 1.35 minutes using method I.

### Step V: 4-tert-Butyl-6-(4-methylphenyl)-2-iodo-pyridin-3-ol

The intermediate was prepared according to General Procedure 9 using 4-tert-butyl-6-(4-methylphenyl)-2-iodo-pyridin-3-amine (1.20 g, 3.28 mmol) in TFA (5.6 mL) and isopentyl nitrite (2.64 mL, 19.66 mmol) affording the title compound. 4-tert-Butyl-6-(4-methylphenyl)-2-iodo-pyridin-3-ol (1.20 g, 99% yield). ESI-MS m/z calc. 367.0433. found 366.0 (M−1)⁻; Retention time: 1.41 minutes using method I.

### Step VI: Ethyl 7-tert-butyl-5-(p-tolyl)furo[3,2-b]pyridine-2-carboxylate

The intermediate was prepared according to General Procedure 10 using 4-tert-butyl-6-(4-methylphenyl)-2-iodo-pyridin-3-ol (1.20 g, 3.27 mmol) in THF (6.13 mL), potassium carbonate (3.16 g, 22.90 mmol), PdCl₂(PPh₃)₂ (92 mg, 0.13 mmol) and CuI (50 mg, 0.26 mmol). Then ethyl prop-2-ynoate (1.32 mL, 13.07 mmol) in THF (3.1 mL) was added affording the title compound. Ethyl 7-tert-butyl-5-(p-tolyl)furo[3,2-b]pyridine-2-carboxylate (260 mg, 24% yield). ESI-MS m/z calc. 337.1678, found 338.3 (M+1)⁺; Retention time: 1.48 minutes using method I.

### Step VII: 7-(tert-Butyl)-5-(p-tolyl)furo[3,2-b]pyridine-2-carboxylic acid

The intermediate was prepared according to General Procedure 11 using 7-tert-butyl-5-(p-tolyl)furo[3,2-b]pyridine-2-carboxylate (60 mg, 0.18 mmol) in dioxane (920 μL) and LiOH (178 mL of 2M, 0.36 mmol) affording the title compound. 7-(tert-Butyl)-5-(p-tolyl)furo[3,2-b]pyridine-2-carboxylic acid (54 mg, 98% yield). ¹H NMR (400 MHz, DMSO-d6) δ 8.05-7.97 (m, 2H), 7.74 (s, 1H), 7.68 (s, 1H), 7.35-7.29 (m, 2H), 2.37 (s, 3H), 1.53 (s, 9H). ESI-MS m/z calc. 309.1365, found 311.07 (M+1)⁺; Retention time: 3.22 minutes using method A.

### Step VIII: tert-Butyl 4-[7-tert-butyl-5-(p-tolyl)furo[3,2-b]pyridine-2-carbonyl]-3,3-dimethyl-piperazine-1-carboxylate

The intermediate was prepared according to General Procedure 1 using 7-tert-butyl-5-(p-tolyl)furo[3,2-b]pyridine-2-carboxylic acid (50 mg, 0.16 mmol), DMF (0.5 mL), HATU (74 mg, 0.20 mmol), tert-butyl 3,3-dimethylpiperazine-1-carboxylate (39 mg, 0.18 mmol) and Hünig's base (99 μL, 0.57 mmol) affording the title compound. tert-Butyl 4-[7-tert-butyl-5-(p-tolyl)furo[3,2-b]pyridine-2-carbonyl]-3,3-dimethyl-piperazine-1-carboxylate (80 mg, 98% yield). ESI-MS m/z calc. 505.2941, found 506.3 (M+1)⁺; Retention time: 1.52 minutes using method I.

### Step IX: [7-tert-Butyl-5-(p-tolyl)furo[3,2-b]pyridin-2-yl]-(2,2-dimethylpiperazin-1-yl)methanone hydrochloride (Intermediate X)

The intermediate was prepared according to General Procedure 2 using tert-butyl 4-[7-tert-butyl-5-(p-tolyl)furo[3,2-b]pyridine-2-carbonyl]-3,3-dimethyl-piperazine-1-carboxylate (80 mg, 0.156 mmol) and 4N HCl solution in 1,4-dioxane (1.18 mL, 4.70 mmol) affording the title compound. [7-tert-Butyl-5-(p-tolyl)furo[3,2-b]pyridin-2-yl]-(2,2-dimethylpiperazin-1-yl)methanone hydrochloride (75 mg, 99% yield). ¹H NMR (400 MHz, DMSO-d6) δ 8.00 (d, J=8.2 Hz, 2H), 7.71 (s, 1H), 7.46 (s, 1H), 7.34-7.28 (m, 2H), 3.49-3.43 (m, 2H), 2.89-2.83 (m, 2H), 2.64 (s, 2H), 2.37 (s, 3H), 1.52 (s, 9H), 1.44 (s, 6H). ESI-MS m/z calc. 405.24163, found 406.4 (M+1)⁺; Retention time: 2.74 minutes using method A.

### Preparation of Intermediate Y: 7-tert-Butyl-5-(4-chloro-3-fluoro-phenyl)furo[3,2-b]pyridine-2-carboxylic acid

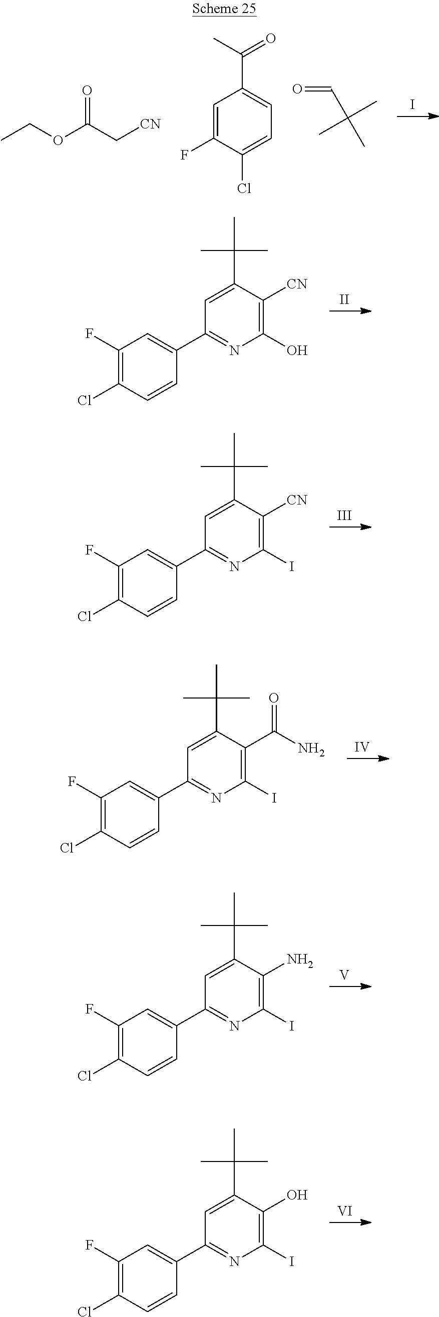

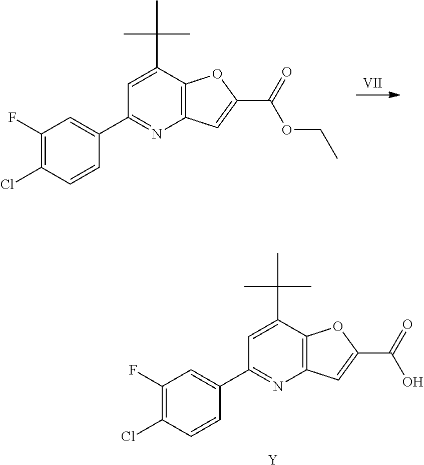

### Step I: 4-tert-Butyl-6-(4-chloro-3-fluoro-phenyl)-2-hydroxy-pyridine-3-carbonitrile

Using 1-(4-chloro-3-fluoro-phenyl)ethanone (11.1 g, 64.32 mmol) as the starting material and following General Procedure 4, the title compound (7.16 g, 47% yield) was obtained as a yellow solid after a final trituration in MeOH (40 mL). ¹H NMR (400 MHz. CDCl₃) δ 7.73 (dd, J=8.6, 2.1 Hz, 1H), 7.69-7.61 (m, 2H), 6.70 (s, 1H), 1.55 (s, 9H). ¹⁹F NMR (376 MHz, CDCl₃) δ −112.44 (dd, J=9.6, 7.3 Hz). ESI-MS m/z calc. 304.07788, found 305.24 (M+1)⁺; Retention time: 1.79 minutes using method C.

### Step II: 4-tert-Butyl-6-(4-chloro-3-fluoro-phenyl)-2-iodo-pyridine-3-carbonitrile

Using 4-tert-butyl-6-(4-chloro-3-fluoro-phenyl)-2-hydroxy-pyridine-3-carbonitrile (6.59 g, 21.62 mmol) as the starting material in MeCN (66 mL) and following General Procedure 5 using triflic acid (2.30 mL, 25.94 mmol) in the second step, the crude product was obtained. The residue was purified by flash chromatography on silica gel eluting with 0-60% DCM: Hexanes. The title compound (4.52 g, 50% yield) was obtained as a white solid. ¹H NMR (400 MHz, CDCl₃) δ 7.85 (dd, J=10.1, 2.1 Hz, 1H), 7.74 (ddd, J=8.4, 2.1, 0.8 Hz, 1H), 7.71 (s, 1H), 7.52 (dd, J=8.4, 7.4 Hz, 1H), 1.56 (s, 9H). ¹⁹F NMR (376 MHz, CDCl₃) δ −113.81 (dd, J=10.1, 7.4 Hz). ESI-MS m/z calc. 413.97958, found 415.2 (M+1)⁺; Retention time: 3.03 minutes using method B.

### Step III: 4-tert-Butyl-6-(4-chloro-3-fluoro-phenyl)-2-iodo-pyridine-3-carboxamide

Using 4-tert-butyl-6-(4-chloro-3-fluoro-phenyl)-2-iodo-pyridine-3-carbonitrile (4.47 g, 10.78 mmol) as the starting material and following General Procedure 7, the reaction mixture was neutralized using potassium carbonate in water (27.0 g in 170 mL). The resulting white precipitate was extracted with CHCl₃/iPrOH mixture (4:1, 3×120 mL). The combined organic extracts were washed with brine (60 mL), dried over MgSO₄, filtered and concentrated, affording the title compound (4.64 g, 99% yield) as an off-white solid. ¹H NMR (400 MHz, CDCl₃) δ 7.79 (dd, J=10.2, 2.0 Hz, 1H), 7.71-7.65 (m, 2H), 7.49 (dd, J=8.4, 7.4 Hz, 1H), 6.01 (broad s, 1H), 5.78 (broad s, 1H), 1.49 (s, 9H). ¹⁹F NMR (376 MHz, CDCl₃) δ −114.49 (dd, J=10.2, 7.4 Hz). ESI-MS m/z calc. 431.99017, found 433.22 (M+1)⁺; Retention time: 1.89 minutes using method C.

### Step IV: 4-tert-Butyl-6-(4-chloro-3-fluoro-phenyl)-2-iodo-pyridin-3-amine

Using 4-tert-butyl-6-(4-chloro-3-fluoro-phenyl)-2-iodo-pyridine-3-carboxamide (1.00 g, 2.31 mmol) as the starting material and following General Procedure 8, the title compound (915 mg, 98% yield) was obtained as a light brown solid which was used in the next step without purification. ¹H NMR (400 MHz, CDCl₃) δ 7.70 (dd, J=10.6, 2.1 Hz. 1H), 7.60 (ddd, J=8.4, 2.1, 0.8 Hz, 1H), 7.44 (s, 1H), 7.40 (dd, J=8.3, 7.6 Hz 1H), 4.51 (broad s, 2H), 1.45 (s, 9H). ¹⁹F NMR (376 MHz, CDCl₃) δ −115.55 (dd, J=10.6, 7.5 Hz). ESI-MS m/z calc. 403.99524, found 405.21 (M+1)⁺; Retention time: 2.52 minutes using method C.

### Step V: 4-tert-Butyl-6-(4-chloro-3-fluoro-phenyl)-2-iodo-pyridin-3-ol

The reaction was performed using 4-tert-butyl-6-(4-chloro-3-fluoro-phenyl)-2-iodo-pyridin-3-amine (2.27 g, 5.61 mmol) as the starting material and following General Procedure 9 with isopentyl nitrite (2.01 g, 17.12 mmol). After the hydrolysis step, the reaction mixture was diluted with EtOAc and H₂O (10 mL each) and the layers were separated. The aqueous layer was extracted with EtOAc (2×50 mL). The combined organic extracts are washed with brine (50 mL), dried over MgSO₄, filtered and concentrated under reduced pressure. The residue was purified by flash chromatography on silica gel eluting with 0-60% DCM: Hexanes. The title compound (1.17 g, 51% yield) was obtained as a white solid. ¹H NMR (400 MHz, CDCl₃) δ 7.72 (dd, J=10.5, 2.0 Hz, 1H), 7.65-7.58 (m, 1H), 7.47 (s, 1H), 7.46-7.39 (m, 1H), 5.65 (s, 1H), 1.43 (s, 9H). ¹⁹F NMR (376 MHz, CDCl₃) δ −115.16 (dd, J=10.4, 7.6 Hz). ESI-MS m/z calc. 404.97928. found 406.19 (M+1)⁺; Retention time: 1.75 minutes using method I.

### Step VI: Ethyl 7-tert-butyl-5-(4-chloro-3-fluoro-phenyl)furo[3,2-b]pyridine-2-carboxylate

Using 4-tert-butyl-6-(4-chloro-3-fluoro-phenyl)-2-iodo-pyridin-3-ol (1.21 g, 2.98 mmol) as the starting material and following General Procedure 10, the reaction mixture was poured carefully into a flask containing 1N HCl (42 mL) and EtOAc (75 mL). The layers were separated and the aqueous layer was extracted with EtOAc (2×25 mL). The combined organic extracts were washed with half-saturated brine (50 mL), dried over MgSO₄ and filtered through Celite®, rinsing with EtOAc and concentrated. The residue was purified by flash chromatography on silica gel eluting with 0-10% EtOAc: Hexanes. The mixed fractions were combined, concentrated and purified again by flash chromatography on silica gel eluting with 0-60% DCM: Hexanes. The title compound (408 mg, 36% yield) was obtained as a pale yellow solid. ¹H NMR (400 MHz, CDCl₃) δ 7.85 (dd, J=10.4, 2.0 Hz, 1H), 7.74 (dd, J=8.4, 1.7 Hz, 1H), 7.67 (s, 1H), 7.61 (s, 1H), 7.56-7.46 (m, 1H), 4.47 (q, J=7.1 Hz, 2H), 1.58 (s, 9H), 1.45 (t, J=7.1 Hz, 3H). ¹⁹F NMR (376 MHz, CDCl₃) δ −115.06 (dd, J=10.3, 7.6 Hz). ESI-MS m/z calc. 375.10376, found 376.02 (M+1)⁺; Retention time: 1.64 minutes using method I.

### Step VII: 7-tert-Butyl-5-(4-chloro-3-fluoro-phenyl)furo[3,2-b]pyridine-2-carboxylic acid

Using ethyl 7-tert-butyl-5-(4-chloro-3-fluoro-phenyl)furo[3,2-b]pyridine-2-carboxylate (398 mg, 1.06 mmol) as the starting material and following General Procedure 11, the reaction mixture was neutralized with 1N HCl (2.2 mL), and diluted with DCM (20 mL) and H₂O (10 mL). The layers were separated. The aqueous layer was further diluted with H₂O (10 mL), acidified with 1N HCl (0.2 mL) and extracted with DCM (20 mL, 2×10 mL). The combined organic extracts were dried over MgSO₄, filtered and concentrated, affording the title compound (417 mg, 100% yield) as a pale yellow solid. ¹H NMR (400 MHz, CDCl₃ and DMSO-d6) δ 7.72 (dd, J=10.4, 2.0 Hz, 1H), 7.61 (dd, J=8.4, 2.1 Hz, 1H), 7.51 (s, 1H), 7.47 (s, 1H), 7.41-7.32 (m, 1H), 1.44 (s, 9H). ¹⁹F NMR (376 MHz, CDCl₃ and DMSO-d6) δ −115.37 (dd, J=10.4, 7.6 Hz). ESI-MS m/z calc. 347.07245, found 348.34 (M+1)⁺; Retention time: 1.18 minutes using method 1.

### Preparation of Intermediate Z: 3-[4-[5-(4-Fluorophenyl)-7-isopropyl-furo[3,2-b]pyridine-2-carbonyl]-3,3-dimethyl-piperazine-1-carbonyl]cyclobutanone

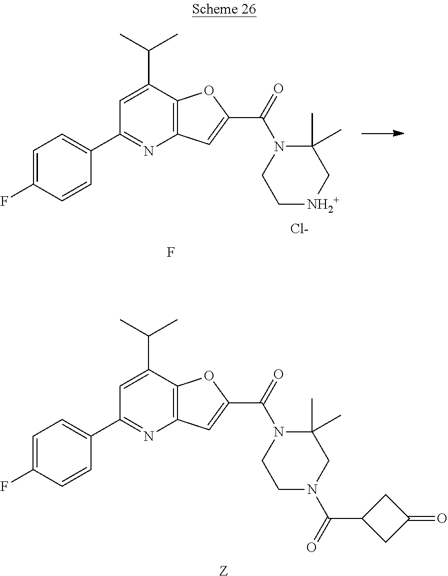

The intermediate was prepared according to General Procedure 3 using (2,2-dimethylpiperazin-1-yl)-[5-(4-fluorophenyl)-7-isopropropyl-furo[3,2-b]pyridin-2-yl]methanone hydrochloride (300 mg, 0.70 mmol), HATU (317 mg, 0.83 mmol), DMF (2.8 mL). Hünig's base (484 μL, 2.78 mmol) and 3-oxocyclobutanecarboxylic acid (87 mg, 0.76 mmol) affording the title compound. 3-[4-[5-(4-Fluorophenyl)-7-isopropyl-furo[3,2-b]pyridine-2-carbonyl]-3,3-dimethyl-piperazine-1-carbonyl]cyclobutanone (332 mg, 97% yield). ESI-MS m/z calc. 491.22205, found 492.77 (M+1)⁺; Retention time: 1.56 minutes using method C.

### Preparation of Intermediate AA: [7-tert-Butyl-5-(4-chlorophenyl)furo[3,2-b]pyridin-2-yl]-(2,2-dimethylpiperazin-1-yl)methanone hydrochloride

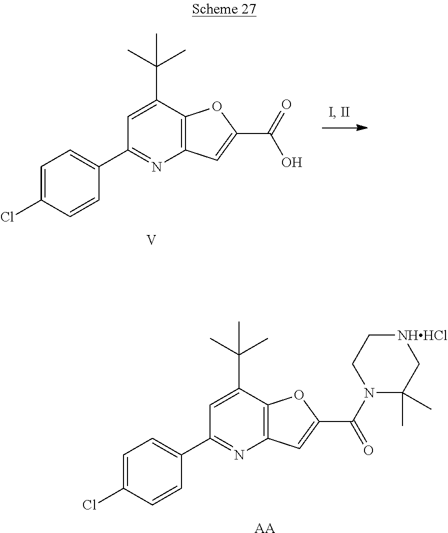

### Step I: tert-Butyl 4-[7-tert-butyl-5-(4-chlorophenyl)furo[3,2-b]pyridine-2-carbonyl]-3,3-dimethyl-piperazine-1-carboxylate

The intermediate was prepared according to General Procedure 1 using a solution of Intermediate V (396 mg, 1.20 mmol) in DMF (5 mL), HATU (548 mg, 1.441 mmol), tert-butyl 3,3-dimethylpiperazine-1-carboxylate (283 μL, 1.32 mmol) and DIPEA (732 μL, 4.20 mmol) affording the title compound (609 mg, 96% yield) as a beige solid, which was used directly in the next step. ESI-MS m/z calc. 525.23944, found 526.55 (M+1)⁺; Retention time: 3.26 minutes using method B.

### Step II: [7-tert-Butyl-5-(4-chlorophenyl)furo[3,2-b]pyridin-2-yl]-(2,2-dimethylpiperazin-1-yl)methanone hydrochloride (Intermediate AA)

Using tert-butyl 4-[7-tert-butyl-5-(4-chlorophenyl)furo[3,2-b]pyridine-2-carbonyl]-3,3-dimethyl-piperazine-1-carboxylate (609 mg, 1.16 mmol) as the starting material and following General Procedure 2 with the exception that the reaction was performed without DCM and using HCl in dioxane (8.7 mL of 4 M, 34.80 mmol). The title compound (642 mg, 100% yield) was obtained as a pale yellow solid, which was used directly in the next step. ESI-MS m/z calc. 425.187, found 426.45 (M+1)⁺; Retention time: 1.77 minutes using method C.

### Preparation of Intermediate BB: (3,3-Dimethylpiperazin-1-yl)-(1H-1,2,4-triazol-3-yl)methanone (bis hydrochloride)

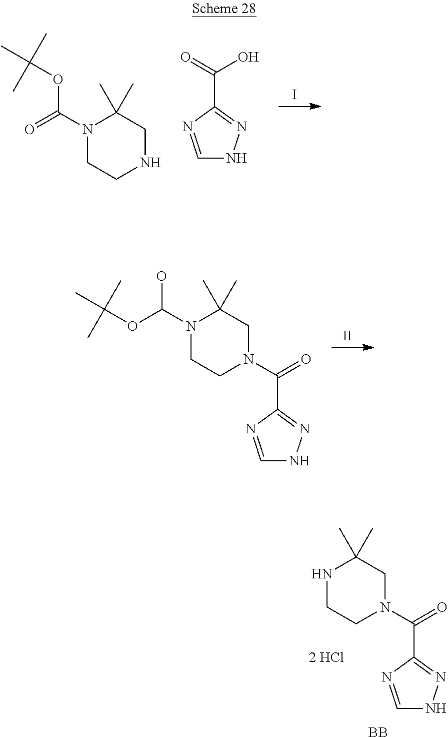

### Step I: tert-Butyl 2,2-dimethyl-4-(1H-1,2,4-triazole-3-carbonyl)piperazine-1-carboxylate

To a mixture of 1H-1,2,4-triazole-3-carboxylic acid (118 mg, 1.044 mmol) and HATU (429 mg, 1.13 mmol) in DMF (2.2 mL) was added DIPEA (325 μL, 1.87 mmol) and the mixture was stirred for 15 min. Then tert-butyl 2,2-dimethylpiperazine-1-carboxylate (200 mg, 0.93 mmol) is added followed by DIPEA (240 μL, 1.38 mmol) and the mixture was stirred for another 90 min. Saturated NH₄Cl was added (5 mL), followed by EtOAc (10 mL). The layers were separated and the aqueous layer extracted with EtOAc (2×5 mL). The combined organic extracts were washed with brine (5 mL), dried over Na₂SO₄, filtered and concentrated. The residue was purified by flash chromatography on silica gel eluting with 0-15% MeOH: DCM affording the title compound (263 mg, 91% yield) as an off-white solid. ESI-MS m/z calc. 309.18008, found 310.35 (M+1)⁺ Retention time: 1.22 minutes using method C.

### Step II: (3,3-Dimethylpiperazin-1-yl)-(1H-1,2,4-triazol-3-yl)methanone bis-hydrochloride (Intermediate BB)

tert-Butyl 2,2-dimethyl-4-(1H-1,2,4-triazole-3-carbonyl)piperazine-1-carboxylate (263 mg, 0.85 mmol) was stirred in a solution of HCl in dioxane (6.38 mL of 4 M, 25.51 mmol) for 1 h. Upon completion, the reaction was concentrated to dryness, affording the title compound (310 mg, 100% yield) as yellow solid which was used in the next step without further purification. ESI-MS m/z calc. 209.12766, found 210.30 (M+1)⁺ Retention time: 0.30 minutes using method C.

### Preparation of Intermediate CC: 3,3-Dimethyl-1-(1H-pyrazol-4-yl)piperazin-2-one

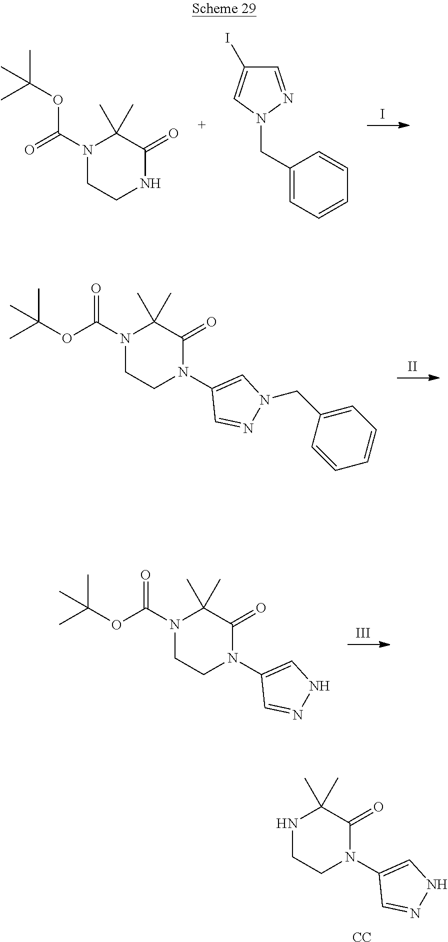

### Step I: tert-Butyl 4-(1-benzylpyrazol-4-yl)-2,2-dimethyl-3-oxo-piperazine-1-carboxylate

To a solution of tert-butyl 2,2-dimethyl-3-oxo-piperazine-1-carboxylate (1.00 g, 4.38 mmol) in toluene (20 mL) were added N,N′-dimethylethane-1,2-diamine (140 μL, 1.32 mmol), 1-benzyl-4-iodo-pyrazole (1.87 g, 6.58 mmol), CuI (125 mg, 0.66 mmol) and tert-butoxypotassium (983 mg, 8.76 mmol). The mixture was degassed and then heated under nitrogen atmosphere at 120° C. for 2 days. Then the mixture was diluted with EtOAc (100 mL), washed with water and brine consecutively, filtered and concentrated to dryness. The residue was purified by flash chromatography on silica gel eluting with 0-80% EtOAc: Hexanes affording tert-butyl 4-(1-benzylpyrazol-4-yl)-2,2-dimethyl-3-oxo-piperazine-1-carboxylate (1.15 g, 68% yield) as a white solid. ¹H NMR (400 MHz, Chloroform-d) δ 8.05 (d, J=0.8 Hz, 1H), 7.51 (d, J=0.8 Hz, 1H), 7.35-7.27 (m, 3H), 7.24-7.20 (m, 2H), 5.24 (s, 2H), 3.75 (ddd, J=5.8, 3.4, 1.2 Hz, 2H), 3.70-3.56 (m, 1H), 1.69 (s, 6H), 1.49 (s, 9H).

### Step II: tert-Butyl 2,2-dimethyl-3-oxo-4-(1H-pyrazol-4-yl)piperazine-1-carboxylate

To a solution of tert-butyl 4-(1-benzylpyrazol-4-yl)-2,2-dimethyl-3-oxo-piperazine-1-carboxylate (1.15 g, 2.99 mmol) in EtOH (10 mL) was added 20% Pd(OH)₂/C (100 mg, 0.14 mmol) and heated at 85° C. under H₂ (1 atm) overnight. After cooling to room temperature, the reaction mixture was filtered, and the solvent was removed under reduced pressure affording tert-butyl 2,2-dimethyl-3-oxo-4-(1H-pyrazol-4-yl)piperazine-1-carboxylate (880 mg, 100% yield) as a white solid. ¹H NMR (400 MHz. Chloroform-d) δ 7.91 (s, 2H), 3.87-3.76 (m, 2H), 3.76-3.70 (m, 2H), 1.72 (s, 6H), 1.50 (s, 9H).

### Step III: 3,3-Dimethyl-1-(1H-pyrazol-4-yl)piperazin-2-one (Intermediate CC)

To a solution of tert-butyl 2,2-dimethyl-3-oxo-4-(1H-pyrazol-4-yl)piperazine-1-carboxylate (880 mg, 2.99 mmol) in DCM (4 mL) was added with 4N HCl/dioxane (3 mL of 4 M, 12.00 mmol). The mixture was stirred at room temperature for 2 h. Upon completion, the volatiles were removed under reduced pressure. The resulting residue was dissolved in water (30 mL), neutralized with sat. aq. NaHCO₃ and extracted with DCM (30 mL×10). The combined organic extracts were washed with brine, dried over sodium sulfate, filtered and concentrated under reduced pressure affording 3,3-dimethyl-1-(1H-pyrazol-4-yl)piperazin-2-one (260 mg, 45% yield) as a white solid. ¹H NMR (400 MHz, Methanol-d4) δ 7.96 (d, J=10.0 Hz, 1H), 7.75 (s, 1H), 3.74 (t, J=5.6 Hz, 2H), 3.18 (t, J=5.6 Hz, 2H), 1.40 (s, 6H).

### Preparation of Intermediate DD: 3,3-Dimethyl-1-(1H-pyrazol-4-yl)piperazin-2-one

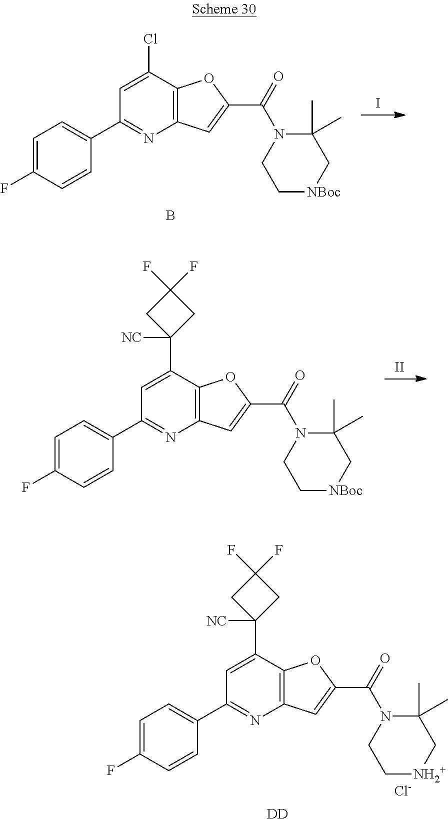

### Step I: tert-butyl 4-[7-(1-cyano-3,3-difluoro-cyclobutyl)-5-(4-fluorophenyl)furo[3,2-b]pyridine-2-carbonyl]-3,3-dimethyl-piperazine-1-carboxylate

To a solution of tert-Butyl 4-[7-chloro-5-(4-fluorophenyl)furo[3,2-b]pyridine-2-carbonyl]-3,3-dimethyl-piperazine-1-carboxylate (Intermediate B) (460 mg, 0.94 mmol) in tetrahydrofuran (4.715 mL) under a nitrogen atmosphere was added 3,3-difluorocyclobutanecarbonitrile (115 mg, 0.98 mmol) and the solution was cooled to −78° C. A solution of (bis(trimethylsilyl)amino)lithium (1.320 mL of 1 M. 1.320 mmol) was added dropwise and the solution was stirred and warmed slowly to room temperature over 16 h. The reaction mixture was diluted with water and EtOAc, the phases were separated and the aqueous phase was extracted twice with EtOAc. The combined organic phase was washed with brine, dried over MgSO4, filtered and the filtrate evaporated under reduced pressure. The residue was purified by silica gel chromatography affording the title compound. tert-butyl 4-[7-(1-cyano-3,3-difluoro-cyclobutyl)-5-(4-fluorophenyl)furo[3,2-b]pyridine-2-carbonyl]-3,3-dimethyl-piperazine-1-carboxylate (270 mg, 0.47 mmol, 50%) ESI-MS m/z calc. 568.22974, found 1.82 (M+1)⁺; Retention time: 569.37 minutes using method C.

### Step II: tert-butyl 4-[7-(1-cyano-3,3-difluoro-cyclobutyl)-5-(4-fluorophenyl)furo[3,2-b]pyridine-2-carbonyl]-3,3-dimethyl-piperazine-1-carboxylate (Intermediate DD)

The intermediate was prepared according to General Procedure 2 using tert-butyl 4-[7-(1-cyano-3,3-difluoro-cyclobutyl)-5-(4-fluorophenyl)furo[3,2-b]pyridine-2-carbonyl]-3,3-dimethyl-piperazine-1-carboxylate (270 mg, 0.4749 mmol) and 4N HCl solution in 1,4-dioxane (2.4 mL, 9.5 mmol) affording the title compound. [7-tert-Butyl-5-(p-tolyl)furo[3,2-b]pyridin-2-yl]-(2,2-dimethylpiperazin-1-yl)methanone hydrochloride (75 mg, 99% yield). ¹H NMR (400 MHz, DMSO-d6) δ 8.00 (d, J=8.2 Hz, 2H), 7.71 (s, 1H), 7.46 (s, 1H), 7.34-7.28 (m, 2H), 3.49-3.43 (m, 2H), 2.89-2.83 (m, 2H), 2.64 (s. 2H), 2.37 (s, 3H), 1.52 (s, 9H), 1.44 (s, 6H). ESI-MS m/z calc. 405.24163, found 406.4 (M+1)⁺; Retention time: 2.74 minutes using method C.

### Preparation of Intermediate EE: 4-[7-chloro-5-(4-fluorophenyl)furo[3,2-b]pyridine-2-carbonyl]-3,3-dimethyl-piperazin-2-one

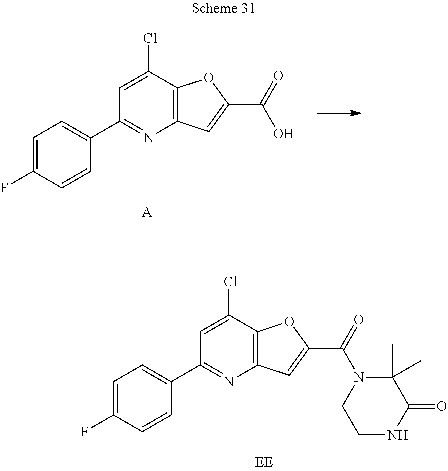

7-Chloro-5-(4-fluorophenyl)furo[3,2-b]pyridine-2-carboxylic acid (Intermediate A) (2.28 g, 7.817 mmol) was dissolved in DMF (39 mL) before HATU (3.567 g, 9.380 mmol), 3,3-dimethylpiperazin-2-one (1.152 g, 8.990 mmol) and Hünig's base (4.765 mL, 27.36 mmol) were added and the solution was stirred at room temperature for 1 h. The reaction mixture was diluted with water, the phases were separated and the aqueous phase was extracted twice with EtOAc. The combined organic phase was washed with brine, dried over MgSO4, filtered and the filtrate evaporated under reduced pressure. The residue was purified by silica gel chromatography affording the title compound. 4-[7-chloro-5-(4-fluorophenyl)furo[3,2-b]pyridine-2-carbonyl]-3,3-dimethyl-piperazin-2-one (3.2 g, 7.964 mmol, 101.9%) ESI-MS m/z calc. 401.09424, found 402.29 (M+1)⁺; Retention time: 2.89 minutes using method C.

### Preparation of Intermediate FF

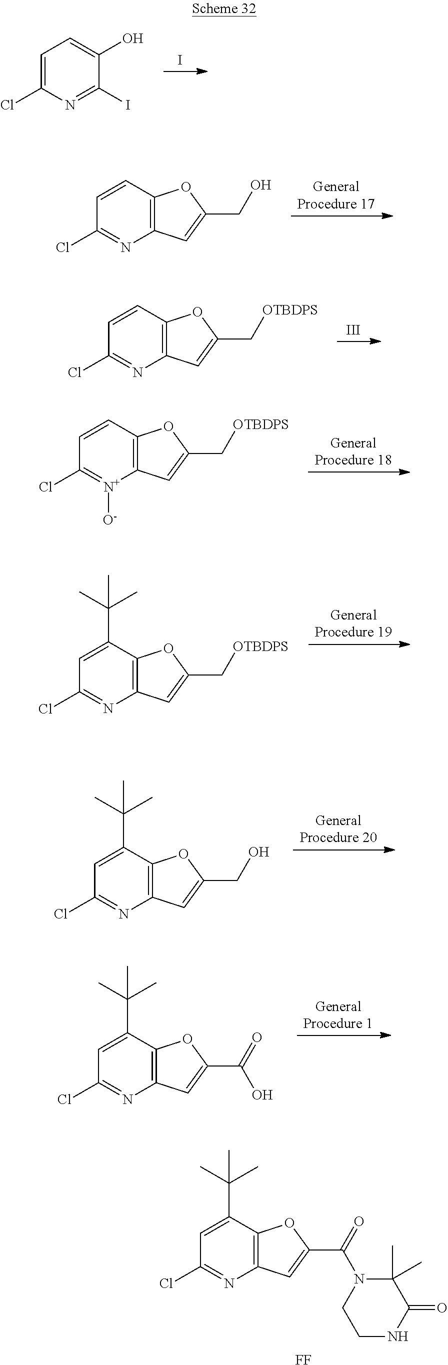

### Step I: (5-chlorofuro[3,2-b]pyridin-2-yl)methanol

To a round bottom flask containing a solution of 6-chloro-2-iodo-pyridin-3-ol (50 g, 195.7 mmol) in 1,4-dioxane (150.0 mL) was added DIPEA (63.2 g, 85.2 mL, 489.2 mmol), PdCl₂(PPh₃)₂ (6.87 g, 9.79 mmol) and CuI (3.73 g. 19.6 mmol). A solution of prop-2-yn-1-ol (14.3 g, 14.8 mL, 254.4 mmol) in 1,4-dioxane (100 mL) was added dropwise over 45 minutes at 75° C. and stirring was pursued for 18 h. The solution was cooled to room temperature, filtered on a Celite pad and the filtrate concentrated under reduced pressure. The filtrate was diluted with water and ethyl acetate and the phases were separated. The aqueous phase was washed twice with ethyl acetate and the combined organic phase was washed with brine, dried over MgSO₄, filtered and the filtrate evaporated under reduced pressure. The residue was purified by silica gel chromatography affording the title compound as a tan solid. (5-chlorofuro[3,2-b]pyridin-2-yl)methanol (27.04 g, 147.3 mmol, 75.3%) ¹H NMR (400 MHz. CDCl₃) δ 7.66 (d, J=8.6 Hz, 1H), 7.20 (d, J=8.6 Hz, 1H), 6.81 (s, 1H), 4.81 (d, J=6.2 Hz, 2H), 2.26 (t, J=6.3 Hz, 1H). ESI-MS m/z calc. 183.00871, found 184.16 (M+1)⁺; Retention time: 0.86 minutes using method C.

### Step II: tert-butyl-[(5-chlorofuro[3,2-b]pyridin-2-yl)methoxy]-diphenyl-silane

The product was prepared according to General Procedure 17 using (5-chlorofuro[3,2-b]pyridin-2-yl)methanol (12 g, 65.4 mmol), imidazole (11.1 g, 162.9 mmol), tert-butyl-chloro-diphenyl-silane (26.95 g, 98.0 mmol) and DMF (108.9 mL) affording the title compound. tert-butyl-[(5-chlorofuro[3,2-b]pyridin-2-yl)methoxy]-diphenyl-silane (27.58 g, 65.4 mmol, 100%). ESI-MS m/z calc. 421.13, found 422.28 (M+1)⁺; Retention time: 2.34 minutes using method C.

### Step III: tert-butyl-[(5-chloro-4-oxido-furo[3,2-b]pyridin-4-ium-2-yl)methoxy]-diphenyl-silane

tert-butyl-[(5-chlorofuro[3,2-b]pyridin-2-yl)methoxy]-diphenyl-silane (12.0 g, 28.4 mmol) was dissolved in DCM (95 mL). The solution was cooled to 0° C. and m-CPBA (21.03 g, 85.32 mmol) is added. The resulting solution was allowed to warm up to room temperature and stirred overnight. The solution was cooled back to 0° C. and more m-CPBA (4.91 g, 28.4 mmol) was added. The resulting solution was allowed to warm up to room temperature and stirred for 36 h. The solution was cooled back to 0° C. and more m-CPBA (2.45 g, 14.2 mmol) was added. The resulting solution was allowed to warm up to room temperature and stirred overnight. The solution was neutralized with a saturated solution of NaHCO₃, the phase are separated and the aqueous phase was extracted twice with DCM. The combined organic layers were dried over sodium sulfate, filtered and concentrated under reduced pressure affording the title compound which was used in the subsequent step without further purification. tert-butyl-[(5-chlorofuro[3,2-b]pyridin-2-yl)methoxy]-diphenyl-silane (12.46 g, 28.5 mmol. 100%). ESI-MS m/z calc. 437.12, found 438.25 (M+1)⁺; Retention time: 1.05 minutes using method I.

### Step IV: tert-butyl-[(7-tert-butyl-5-chloro-furo[3,2-b]pyridin-2-yl)methoxy]-diphenyl-silane

The product was prepared according to General Procedure 18 using tert-butyl-[(5-chloro-4-oxido-furo[3,2-b]pyridin-4-ium-2-yl)methoxy]-diphenyl-silane (3.32 g, 7.580 mmol), iodocopper (577.4 mg, 3.032 mmol), THF (15.16 mL), trifluoro-tetrahydrofuran-1-ium-1-yl-boron (2.334 g, 16.68 mmol), tert-butyl(chloro)magnesium (15.10 g, 18.19 mL of 1 M, 18.19 mmol), DDQ (189.3 mg, 0.8338 mmol) and DDQ (172.1 mg, 0.7580 mmol) affording the title compound which was used directly in the subsequent step without further purification. tert-butyl-[(7-tert-butyl-5-chloro-furo[3,2-b]pyridin-2-yl)methoxy]-diphenyl-silane (3.62 g. 7.58 mmol, 100%). ESI-MS m/z calc. 477.19, found 478.35 (M+1)⁺; Retention time: 2.16 minutes using method I.

### Step V: (7-tert-butyl-5-chloro-furo[3,2-b]pyridin-2-yl)methanol

The product was prepared according to General Procedure 19 using tert-butyl-[(7-tert-butyl-5-chloro-furo[3,2-b]pyridin-2-yl)methoxy]-diphenyl-silane (3.62 g, 7.58 mmol), TBAF (15.14 mL of 1 M, 15.14 mmol) and THF (18.2 mL) affording the title compound. (7-tert-butyl-5-chloro-furo[3,2-b]pyridin-2-yl)methanol (800 mg, 3.338 mmol, 44.08%). ESI-MS m/z calc. 239.07, found 340.05 (M+1)⁺; Retention time: 1.43 minutes using method C.

### Step VI: 7-tert-butyl-5-chloro-furo[3,2-b]pyridine-2-carboxylic acid (Intermediate FF)

The product was prepared according to General Procedure 20 using (7-tert-butyl-5-chloro-furo[3,2-b]pyridin-2-yl)methanol (800 mg, 3.338 mmol), NMO monohydrate (5.112 g, 33.37 mmol), TPAP (117.3 mg, 0.334 mmol), MeCN (11.2 mL) and iPrOH (2.58 mL, 33.7 mmol) affording the title compound. 7-tert-butyl-5-chloro-furo[3,2-b]pyridine-2-carboxylic acid (710 mg, 84%). ESI-MS m/z calc. 253.05, found 254.27 (M+1)⁺; Retention time: 1.39 minutes using method C.

### Preparation of Intermediate GG: 7-tert-Butyl-5-(4-fluorophenyl)furo[3,2-b]pyridine-2-carboxylic acid

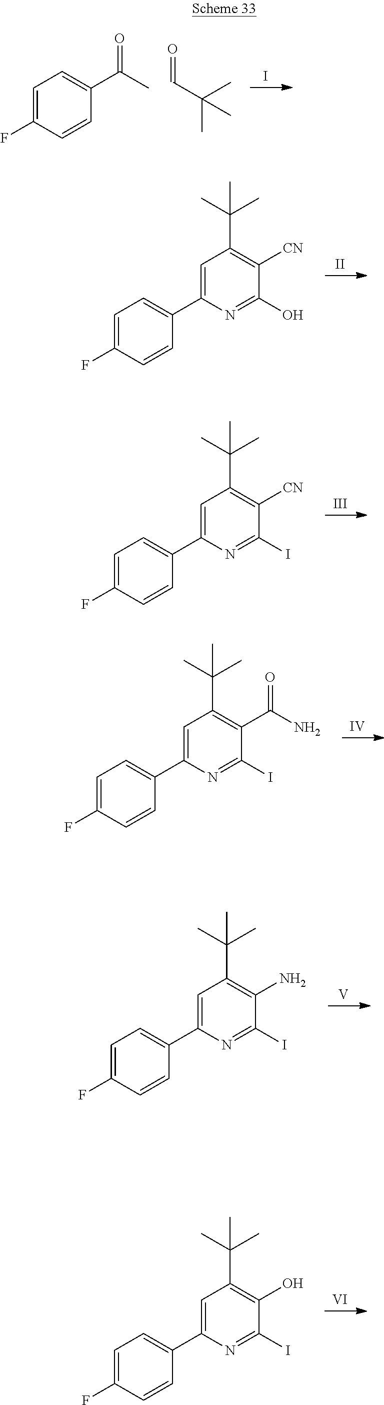

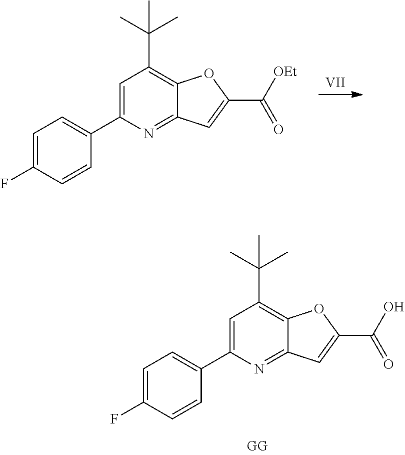

### Step I: 4-tert-Butyl-6-(4-fluorophenyl)-2-hydroxy-pyridine-3-carbonitrile

The intermediate was prepared according to General Procedure 4 using 1-(4-fluorophenyl)ethanone (19.84 g, 143.6 mmol), 2,2-dimethylpropanal (12 mL, 110.5 mmol), ethyl 2-cyanoacetate (11.78 mL, 110.5 mmol), ethanol (110 mL) and ammonium acetate (80.94 g, 1.05 mol). Then DCM (130 mL) and DDQ (20.07 g, 88.40 mmol) were added affording the title compound. 4-(tert-Butyl)-6-(4-fluorophenyl)-2-hydroxynicotinonitrile (14.8 g, 50% yield). ESI-MS m/z calc. 270.11685, found 271.27 (M+1)⁺; Retention time: 1.43 minutes using method C.

### Step II: 4-tert-Butyl-6-(4-fluorophenyl)-2-iodo-pyridine-3-carbonitrile

The intermediate was prepared according to General Procedure 5 using 4-(tert-butyl)-6-(4-fluorophenyl)-2-hydroxynicotinonitrile (10.00 g, 37.00 mmol) and pyridine (3.89 mL, 48.10 mmol) in acetonitrile (37 mL) at 0° C. and using triflic anhydride (7.47 mL, 44.40 mmol). Then NaI (27.73 g, 185.0 mmol) and HCl (4.63 mL of 12 M, 55.50 mmol) were added affording the title compound. 4-tert-Butyl-6-(4-fluorophenyl)-2-iodo-pyridine-3-carbonitrile (9.5 g, 67% yield). ESI-MS m/z calc. 380.0186, found 381.15 (M+1)⁺; Retention time: 2.19 minutes using method C.

### Step III: 4-tert-Butyl-6-(4-fluorophenyl)-2-iodo-pyridine-3-carboxamide

The intermediate was prepared according to General Procedure 7 using 4-(tert-butyl)-6-(4-fluorophenyl)-2-iodonicotinonitrile (800 mg, 2.10 mmol) and sulfuric acid (1.12 mL, 21.04 mmol) affording the title compound. 4-tert-Butyl-6-(4-fluorophenyl)-2-iodo-pyridine-3-carboxamide (843 mg, 100%, yield). ESI-MS m/z calc. 398.02914, found 399.16 (M+1)⁺; Retention time: 1.57 minutes using method C.

### Step IV: 4-tert-Butyl-6-(4-fluorophenyl)-2-iodo-pyridin-3-amine

The intermediate was prepared according to General Procedure 8 using 4-tert-butyl-6-(4-fluorophenyl)-2-iodo-pyridine-3-carboxamide (20.00 g, 50.22 mmol). KOH (19.72 g, 351.5 mmol), water (55 mL) and bromine (3.36 mL, 65.29 mmol), then water (19 mL) and THF (19 mL) affording the title compound. 4-tert-Butyl-6-(4-fluorophenyl)-2-iodo-pyridin-3-amine (17.21 g, 93% yield). ESI-MS m/z calc. 370.0342, found 371.17 (M+1)⁺; Retention time: 2.13 minutes using method C.

### Step V: 4-tert-Butyl-6-(4-fluorophenyl)-2-iodo-pyridin-3-ol

The intermediate was prepared according to General Procedure 9 using 4-tert-butyl-6-(4-fluorophenyl)-2-iodo-pyridin-3-amine (4.99 g, 13.48 mmol) in TFA (24 mL) and isopentyl nitrite (2.72 mL, 20.22 mmol) affording the title compound. 4-tert-Butyl-6-(4-fluorophenyl)-2-iodo-pyridin-3-ol (5.1 g, 100% yield). ESI-MS m/z calc. 371.0182, found 372.29 (M+1)⁺; Retention time: 2.14 minutes using method C.

### Step VI: Ethyl 7-tert-butyl-5-(4-fluorophenyl)furo[3,2-b]pyridine-2-carboxylate

The intermediate was prepared according to General Procedure 10 using 4-tert-butyl-6-(4-fluorophenyl)-2-iodo-pyridin-3-ol (5.1 g, 13.74 mmol) in THF (26 mL), potassium carbonate (13.29 g, 96.18 mmol), PdCl₂(PPh₃)₂ (386 mg, 0.55 mmol) and CuI (209 mg, 1.10 mmol). Then ethyl prop-2-ynoate (5.57 mL, 54.96 mmol) in THF (13 mL) was added affording the title compound. Ethyl 7-tert-butyl-5-(4-fluorophenyl)furo[3,2-b]pyridine-2-carboxylate (348 mg, 62% yield). ESI-MS m/z calc. 341.1427, found 342.61 (M+1)⁺; Retention time: 2.21 minutes using method C.

### Step VII: 7-tert-Butyl-5-(4-fluorophenyl)furo[3,2-b]pyridine-2-carboxylic acid (Intermediate GG)

The intermediate was prepared according to General Procedure 11 using ethyl 7-tert-butyl-5-(4-fluorophenyl)furo[3,2-b]pyridine-2-carboxylate (970 mg, 2.84 mmol) and aqueous LiOH (2.84 mL of 2 M, 5.68 mmol) in dioxane (15 mL) affording the title compound. 7-tert-Butyl-5-(4-fluorophenyl)furo[3,2-b]pyridine-2-carboxylic acid (867 mg, 97% yield). ¹H NMR (400 MHz, DMSO-d6) δ 8.23-8.13 (m, 2H), 7.83-7.77 (m, 2H), 7.39-7.28 (m, 2H), 1.54 (s, 9H). ESI-MS m/z calc. 313.1114, found 314.52 (M+1)⁺; Retention time 1.64 minutes using method C.

### Preparation of Intermediate HH: 3,3-Dimethyl-1-(1H-pyrazol-4-yl)piperazin-2-one

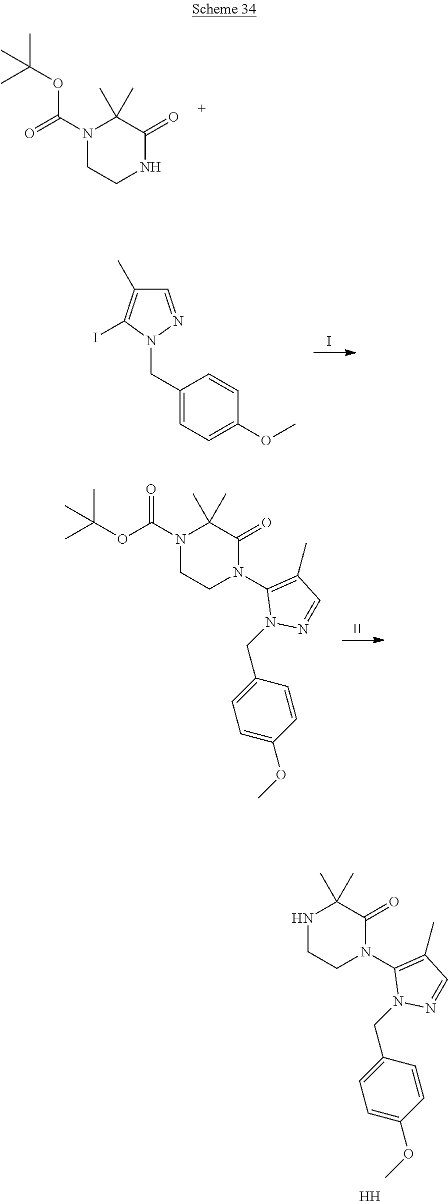

### Step I: tert-butyl 4-[2-[(4-methoxyphenyl)methyl]-4-methyl-pyrazol-3-yl]-2,2-dimethyl-3-oxo-piperazine-1-carboxylate

To a solution of tert-butyl 2,2-dimethyl-3-oxo-piperazine-1-carboxylate (915 mg, 4.01 mmol), 5-iodo-1-[(4-methoxyphenyl)methyl]-4-methyl-pyrazole (1.58 g, 4.81 mmol), iodocopper (381.7 mg, 2.004 mmol), N,N′-dimethylethane-1,2-diamine (353.3 mg, 426.7 μL, 4.008 mmol), potassium phosphate (1.70 g, 8.02 mmol) in DMF (18 mL) was stirred for 3.5 h reaction at 120° C. The reaction mixture was cooled and filtered through Celite. The filtrate was diluted with water and extracted twice with EtOAc. The Combined organic phases were washed with brine, dried over Na₂SO₄ and evaporated in vacuo. The residue was then purified by silica gel chromatorgraphy to afford 4-[2-[(4-methoxyphenyl)methyl]-4-methyl-pyrazol-3-yl]-2,2-dimethyl-3-oxo-piperazine-1-carboxylate (1.44 g, 84%) as a pure white solid compound. 1H NMR (400 MHz, DMSO-d6) δ 7.50-7.45 (m, 1H), 7.21-7.14 (m, 2H), 6.90-6.83 (m, 2H), 5.07 (s, 2H), 3.70 (s, 3H), 3.68-3.56 (m, 4H), 1.77-1.70 (m, 3H), 1.59 (s, 6H), 1.41 (s, 9H). ESI-MS m/z calc. 428.24, found 429.4 (M+1)⁺; Retention time 1.78 minutes using method C.

### Step II: 1-[2-[(4-methoxyphenyl)methyl]-4-methyl-pyrazol-3-yl]-3,3-dimethyl-piperazin-2-one (Hydrochloride salt) (Intermediate HH)

To a solution of tert-butyl 4-[2-[(4-methoxyphenyl)methyl]-4-methyl-pyrazol-3-yl]-2,2-dimethyl-3-oxo-piperazine-1-carboxylate (1.44 g, 3.37 mmol) in DCM (15 mL) was added with 4N HCl/dioxane (4.2 mL of 4 M, 16.8 mmol). The solution was sonicated and Et₂O is added dropwise until complete precipitation of the desired product. The white solid was then filtered and dried under vacuum to give 1-[2-[(4-methoxyphenyl)methyl]-4-methyl-pyrazol-3-yl]-3,3-dimethyl-piperazin-2-one (Hydrochloride salt) (1.09 g, 88.69%). ¹H NMR (400 MHz, DMSO-d6) δ 10.16 (s, 2H), 7.52 (s, 1H), 7.21-7.14 (m, 2H), 6.90-6.83 (m, 2H), 5.08 (s, 2H), 3.86-3.78 (m, 2H), 3.70 (s, 3H), 3.60-3.47 (m, 2H), 1.75 (s, 3H), 1.56 (s, 6H). ESI-MS m/z calc. 328.42, found 329.36 (M+1-HCl)⁺; Retention time 0.77 minutes using method C.

### Preparation of Intermediate II: 7-(tert-butyl)-5-(4,4-dimethylcyclohexyl)furo[3,2-b]pyridine-2-carboxylic acid

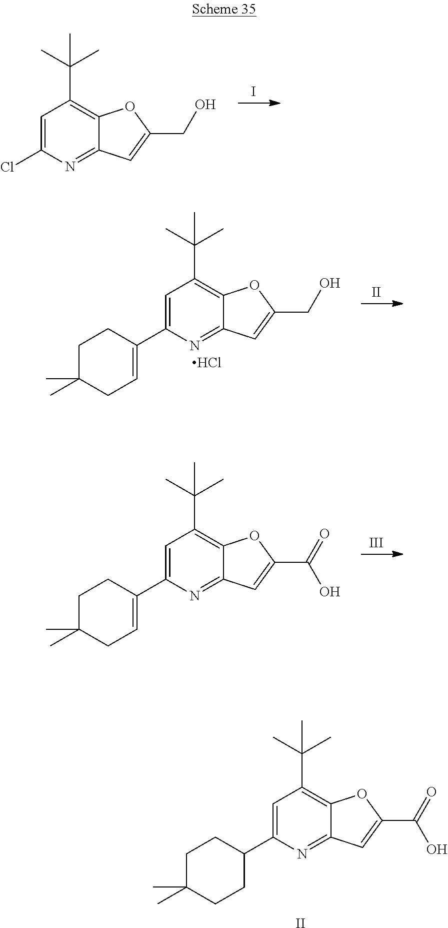

### Step I: (7-(tert-butyl)-5-(4,4-dimethylcyclohex-1-en-1-yl)furo[3,2-b]pyridin-2-yl)methanol

A mixture of (7-tert-butyl-5-chloro-furo[3,2-b]pyridin-2-yl)methanol (100 g, 417.2 mmol), 2-(4,4-dimethylcyclohexen-1-yl)-4,4,5,5-tetramethyl-1,3,2-dioxaborolane (108.4 g, 458.9 mmol), and Na₂CO₃ (134.6 g, 1.270 mol) was suspended in dioxane (2 L) and water (667 mL). The resulting solution was degassed with nitrogen for 5 minutes prior to adding [1,1′-Bis(diphenylphosphino)ferrocene]dichloropalladium(II) complex with dichloromethane (17.04 g, 20.86 mmol). It was then stirred at 90° C. for 18 h. The solvent was distilled, and to the cooled residue is water (500 mL) and EtOAc (2.5 L). The biphasic mixture was filtered through Celite and the layers were separated. The aqueous layer was back-extracted EtOAc (1000 mL). The combined organic layers were washed with brine then dried over MgSO4, filtered then concentrated under reduced pressure to approximately 2 L. The residue was treated with a solution of HCl in dioxane (4.0 M, 160 mL, 640 mmol). Heptane (300.0 mL) was added and the mixture was stirred at RT for 16 h. The solid was filtered, washed with EtOAc and dried under reduced pressure to afford the title compound (118.5 g, 81%) as a brown-beige solid, which was used directly in the next step. ESI-MS m/z calc. 313.2042, found 313.76 (M+1)⁺; Retention time: 1.55 minutes using method C.

### Step II: 7-(tert-butyl)-5-(4,4-dimethylcyclohex-1-en-1-yl)furo[3,2-b]pyridine-2-carboxylic acid

To a mixture of NMO hydrate (118.28 g. 875.18) and TPAP (2.28 g, 6.49 mmol) in acetonitrile (710 mL) was added portionwise (7-(tert-butyl)-5-(4,4-dimethylcyclohex-1-en-1-yl)furo[3,2-b]pyridin-2-yl)methanol (38.7 g, 101.4 mmol) while maintaining the temperature below 35° C. The reaction mixture was stirred at room temperature for 1 h. The solution was cooled to 0° C., i-PrOH (78 mL, 1.0 mol) was added slowly and the reaction was stirred for 15 minutes. The reaction mixture was diluted with water and the volatiles are removed under reduced pressure. The aq. phase was basified to pH 13 using 2N NaOH and extracted with MTBE three times. The combined organic phases were concentrated under reduced pressure and the residue was diluted with water (535 mL). The resulting aqueous solution was acidified to pH 3 with 2N HCl, the product formed was collected by filtration, washed with water and dried under reduced pressure affording the title compound (20.03 g, 60%) as a beige solid, which was used directly in the next step. ESI-MS m/z calc. 327.1834, found 328.2 (M+1)⁺; Retention time: 3.47 minutes using method A.

### Step III: 7-(tert-butyl)-5-(4,4-dimethylcyclohexyl)furo[3,2-b]pyridine-2-carboxylic acid (Intermediate II)

In a Büchi pressure vessel purged with N2 was charged with Pd on carbon (wet base, 5.11 g, 4.80 mmol) followed by a solution of 7-tert-butyl-5-(4,4-dimethylcyclohexen-1-yl)furo[3,2-b]pyridine-2-carboxylic acid (15.59 g, 45.66 mmol) and AcOH (5.5 mL, 96.72 mmol) in THF (100 mL) and then ethanol (400 mL). Nitrogen atmosphere was evacuated under reduced pressure and re-filled with hydrogen 3 times and the suspension was stirred under 80 psi hydrogen pressure at room temperature for 4 days. The suspension was filtered over celite, washed with THF and the volatiles were removed under reduced pressure. This solid was suspended MeOH:Water (1:1) then heated to reflux. The mixture was cooled to RT then stirred for 1 hr in an ice-bath (5-10° C.). The product formed was collected by filtration, washed with water then heptane, and dried under reduced pressure affording the title compound (12.60 g, 80%) as a brown-beige solid, which was used directly in the next step. ESI-MS m/z calc. 329.1991, found 329.77 (M+1)⁺; Retention time: 3.26 minutes using method A.

### Preparation of Intermediate JJ: (7-(tert-butyl)-5-(4,4-dimethylcyclohexyl)furo[3,2-b]pyridin-2-yl)(2,2-dimethylpiperazin-1-yl)methanone hydrochloride

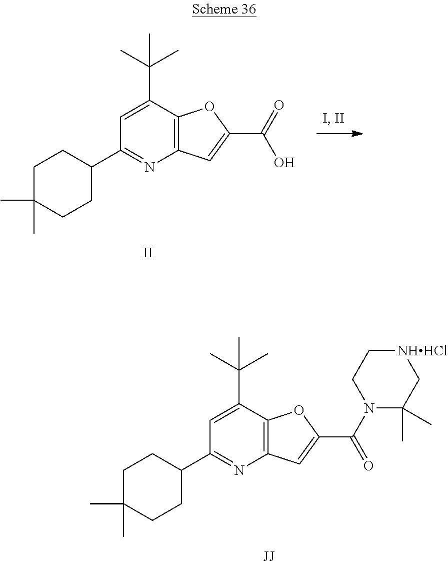

### Step I: tert-butyl 4-(7-(tert-butyl)-5-(4,4-dimethylcyclohexyl)furo[3,2-b]pyridine-2-carbonyl)-3,3-dimethylpiperazine-1-carboxylate

tert-butyl 3,3-dimethylpiperazine-1-carboxylate (19.90 g, 92.86 mmol) and (2,2-dimethylpiperazin-1-yl)methanone hydrochloride (Intermediate II) (27.87 g, 84.60 mmol) were dissolved in DMF (300 mL) before HATU (35.45 g, 93.23 mmol) and DIPEA (27.72 g, 37.36 mL, 214.5 mmol) were added. The solution was stirred at room temperature for 3 h, and then water was added dropwise. The aq. phase was extracted three times with EtOAc. The combined organic phase was washed with water and brine. The organic phase was then dried over Na₂SO₄, filtered and the filtrate evaporated under reduced pressure. The crude product was purified passing through a pad of silica gel (25%-33% EtOAc/hexanes affording the title product (34.28 g, 91% yield) as a yellow orange foamy solid, which was used directly in the next step. 1H NMR (400 MHz, Chloroform-d) δ 7.34 (s, 1H), 7.06 (s, 1H), 3.84 (dd, J=6.6, 4.8 Hz, 2H), 3.67-3.45 (m, 4H), 2.71 (tt, J=10.3, 5.5 Hz, 1H), 1.85-1.77 (m, 4H), 1.77-1.71 (m, 1H), 1.57-1.51 (m, 2H), 1.49 (s, 9H), 1.49 (s, 9H), 1.43-1.31 (m, 3H), 1.01 (s, 3H), 0.97 (s, 3H). ESI-MS m/z calc. 525.3567, found 526.61 (M+1)⁺; Retention time: 3.15 minutes using method A.

### Step II: (7-(tert-butyl)-5-(4,4-dimethylcyclohexyl)furo[3,2-b]pyridin-2-yl)(2,2-dimethylpiperazin-1-yl)methanone hydrochloride (Intermediate JJ)

Using tert-butyl 4-[7-tert-butyl-5-(4,4-dimethylcyclohexyl)furo[3,2-b]pyridine-2-carbonyl]-3,3-dimethyl-piperazine-1-carboxylate (34.2 g, 65.05 mmol) as the starting material and following General Procedure 2 with the exception that the reaction was performed in dioxane instead of DCM and using HCl in dioxane (162.6 mL of 4.0 M, 650.5 mmol). The titoule compound (31.4 g. quantitative) was obtained as an orange foam, which was used directly in the next step. ESI-MS m/z calc. 425.30423, found 426.53 (M+1)⁺; Retention time: 3.14 minutes using method A.

### Preparation of Intermediate KK: [7-tert-Buty-5-(4-chloro-3-fluorophenyl)furo[3,2-b]pyridin-2-yl]-(2,2-dimethylpiperazin-1-yl)methanone hydrochloride

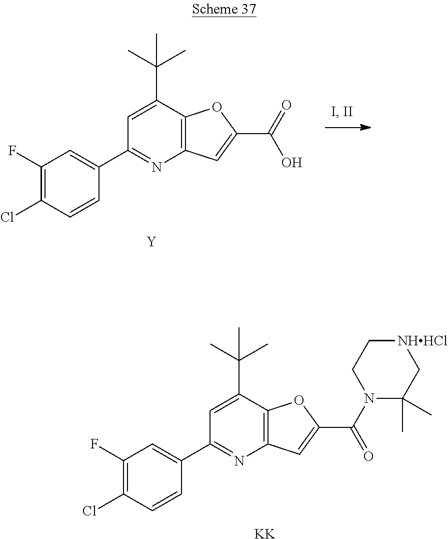

### Step I: tert-Butyl 4-[7-tert-butyl-5-(4-chloro-3-fluorophenyl)furo[3,2-b]pyridine-2-carbonyl]-3,3-dimethyl-piperazine-1-carboxylate

The intermediate was prepared according to General Procedure 1 using a solution of Intermediate Y (10.0 g, 28.3 mmol) in DMF (110 mL), HATU (12.0 g, 31.6 mmol), tert-butyl 3,3-dimethylpiperazine-1-carboxylate (6.64, 31.0 mmol) and DIPEA (12.5 ml, 71.8 mmol) affording the title compound (15.38 g, quantitative yield) as a pale yellow solid, which was used directly in the next step. 1H NMR (400 MHz, DMSO-d) δ 8.16 (dd, J=11.0, 2.0 Hz, 1H), 8.07-7.97 (m, 1H), 7.82 (s, 1H), 7.76-7.64 (m, 1H), 7.51 (s, 1H), 3.85 (s, 2H), 3.57-3.38 (m, 4H), 1.52 (s, 10H), 1.48 (s, 6H), 1.42 (s, 9H). ESI-MS m: calc. 543.2300, found 546.10 (M+1)⁺; Retention time: 1.23 minutes using method J.

### Step II: 17-tert-Butyl-5-(4-chloro-3-fluorophenyl)furo[3,2-b]pyridin-2-yl-(2,2-dimethylpiperazin-1-yl)methanone hydrochloride (Intermediate KK)

Using tert-butyl 4-[7-tert-butyl-5-(4-chlorophenyl)furo[3,2-b]pyridine-2-carbonyl]-3,3-dimethyl-piperazine-1-carboxylate (15.38 g, 28.27 mmol) as the starting material and following General Procedure 2 with the exception that the reaction was performed in dioxane instead of DCM and using HCl in dioxane (35 mL of 4 M, 140 mmol). The title compound (12.80 g, 88% yield) was obtained as a pale yellow solid, which was used directly in the next step. 1H NMR (400 MHz, DMSO-d₆) δ 9.37 (broad s, 2H), 8.17 (dd, J=11.0, 1.9 Hz, 1H), 8.03 (dd, J=8.4, 1.8 Hz, 1H), 7.85 (s, 1H), 7.72 (t, J=8.2 Hz, 1H), 7.62 (s, 1H), 3.90-3.74 (m, 2H), 3.38-3.27 (m, 2H), 3.26-3.15 (m, 2H), 1.58 (s, 6H), 1.53 (s, 9H). ESI-MS m/z calc. 443.1776, found 443.73 (M+1)⁺; Retention time: 0.66 minutes using method J.

### Preparation of Intermediate LL: (7-tert-butyl-5-chloro-furo[3,2-b]pyridin-2-yl-[4-(3-hydroxy-3-methyl-cyclobutanecarbonyl)-2,2-dimethyl-piperazin-1-yl]methanone

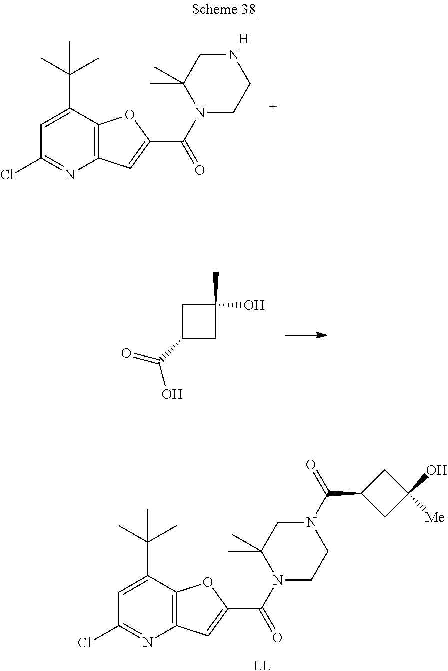

To a solution of (7-tert-butyl-5-chloro-furo[3,2-b]pyridin-2-yl)-(2,2-dimethylpiperazin-1-yl)methanone (dihydrochloride salt) (960 mg, 2.271 mmol) and 3-hydroxy-3-methyl-cyclobutanecarboxylic acid (319.2 mg, 2.453 mmol) in DMF (10.24 mL) was added N-methyl morpholine (1.608 g, 1.75 mL, 15.90 mmol) at ambient temperature. T3P (1.95 g, 3.07 mmol) 50% W/W in DMF was added dropwise and the solution was stirred at room temperature for 30 minutes. A saturated solution of Na_{H}CO3 was added and the aqueous phase was extracted 3 times with EtOAc. The combined organic phase was washed with aq. sat. NH₄Cl and brine, dried over MgSO4 and filtered. The filtrate was evaporated under reduced pressure affording the title compound as a white solid. (7-tert-butyl-5-chloro-furo[3,2-b]pyridin-2-yl)-[4-(3-hydroxy-3-methyl-cyclobutanecarbonyl)-2,2-dimethyl-piperazin-1-yl]methanone (958 mg, 91%) ESI-MS m/z calc. 461.20813, found 462.49 (M+1)+; Retention time: 1.45 minutes using method C.

### Preparation of Intermediate MM: (7-(tert-butyl)-5-(spiro[3.3]heptan-2-yl)furo[3,2-b]pyridin-2-yl)(2,2-dimethylpiperazin-1-yl)methanone hydrochloride salt

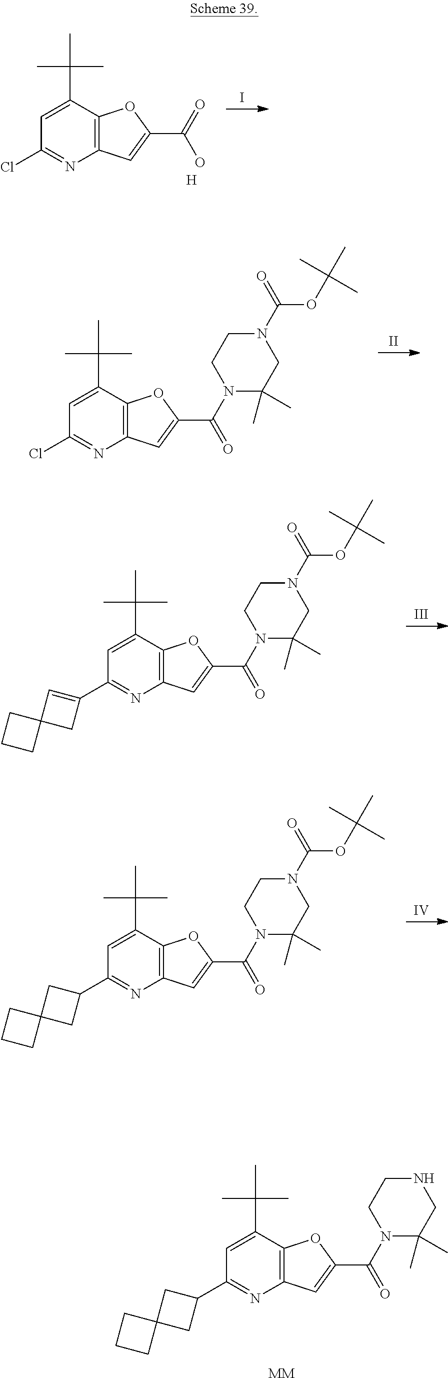

### Step I: tert-butyl-4-(7-tert-butyl-5-chloro-furo[3,2-b]pyridine-2-carbonyl)-3,3-dimethyl-piperazine-1-carboxylate

According to general procedure 1, 7-tert-butyl-5-chloro-furo[3,2-b]pyridine-2-carboxylic acid (5.3 g, 20.9 mmol) in NMP (55 mL) containing DIPEA (9.1 mL, 52.2 mmol) was treated with tert-butyl 3,3-dimethylpiperazine-1-carboxylate (5.03 g, 23.0 mmol) and HATU (8.74 g, 23.0 mmol) and the resulting dark brown clear solution was stirred at RT for 4 h. Water (25 ml) was added and the mixture was stirred for 30 min and filtered. The solid was washed with water (50 ml) and air dried to afford an off-white solid (8.49 g). ¹H NMR (400 MHz, DMSO-d6) δ 7.43 (s, 1H), 7.29 (s, 1H), 3.80 (s, 2H), 3.47 (m, 4H), 1.44 (s, 6H), 1.43 (s, 9H), 1.39 (s, 9H).

### Step II: tert-butyl 4-(7-tert-butyl-5-spiro[3,3]hept-2-en-2-yl-furo[3,2-b]pyridine-2-carbonyl)-3,3-dimethyl-piperazine-1-carboxylate

A suspension of tert-butyl 4-(7-tert-butyl-5-chloro-furo[3,2-b]pyridine-2-carbonyl)-3,3-dimethyl-piperazine-1-carboxylate (5.0 g, 11.1 mmol), 4,4,5,5-tetramethyl-2-spiro[3,3]hept-2-en-2-yl-1,3,2-dioxaborolane (2.93 g, 13.3 mmol) and Na₂CO₃ (13.9 mL of 2 M, 27.8 mmol) in DMF (125 mL) and water (20 mL) was treated with PdCl₂(dppf)₂-DCM (453.6 mg, 0.556 mmol) and heated at 90° C. for 3.5 h. The mixture was allowed to cool to RT, water was added (250 ml) and then stirred for 30 min before filtering. The resulting solid was washed with water (100 ml). The solid was taken up in EtOAc (250 ml), dried (MgSO₄), filtered and concentrated invacuo to afford a black gum. The residue was dissolved in DCM (20 ml) and columned on Rf companion (120 g cartridge), eluting with a Pet ether:EtOAc gradient of 100-0 to 50:50. ¹H NMR (400 MHz, DMSO-d6) δ 7.45 (s, 1H), 7.36 (s, 1H), 6.78 (s, 1H), 3.85 (d, J=6.3 Hz, 2H), 3.51 (d, J⁼7.2 Hz, 4H), 2.84 (s, 2H), 2.26-2.12 (m, 4H), 1.92 (dd, J=9.2, 6.3 Hz, 2H), 1.48 (s, 6H), 1.46 (s, 9H), 1.43 (s, 9H).

### Step III: tert-butyl 4-(7-tert-butyl-5-spiro[3.3]heptan-2-yl-furo[3,2-b]pyridine-2-carbonyl)-3,3-dimethyl-piperazine-1-carboxylate

A mixture of tert-butyl 4-(7-tert-butyl-5-spiro[3,3]hept-2-en-2-yl-furo[3,2-b]pyridine-2-carbonyl)-3,3-dimethyl-piperazine-1-carboxylate (5.2 g, 9.53 mmol) in EtOH (60 mL) and EtOAc (20 mL) was hydrogenated (balloon) with 10% Pd—C (600 mg, 5.638 mmol) overnight. The mixture was filtered on celite and the solvent removed in vacuo to give 4-(7-tert-butyl-5-spiro[3.3]heptan-2-yl-furo[3,2-b]pyridine-2-carbonyl)-3,3-dimethyl-piperazine-1-carboxylate 5.27 g. ESI-MS m/z calc. 509.32535, found: 510.5.

### Step IV: (7-tert-butyl-5-spiro[3.3]heptan-2-yl-furo[3,2-b]pyridin-2-yl)-(2,2-dimethylpiperazin-1-yl)methanone

A solution of tert-butyl 4-(7-tert-butyl-5-spiro[3.3]heptan-2-yl-furo[3,2-b]pyridine-2-carbonyl)-3,3-dimethyl-piperazine-1-carboxylate (6.189 g, 10.20 mmol) in DCM (100 mL) was treated with HCl in Dioxane (25.5 mL of 4 M, 102.0 mmol) and the solution was stirred for 1.5 h. The solution was concentrated in vacuo to afford a brown foam. The residue was partitioned carefully with EtOAc and saturated NaHCO₃. The layers were separated and the organic phase dried (MgSO4), filtered and concentrated in vacuo. The residue was purified by flash column chromatography on silica gel to afford the title compound 2.38 g. 1H NMR (400 MHz, DMSO-d6) δ 7.38 (s, 1H), 7.08 (s, 1H), 3.62-3.50 (m, 1H), 3.47-3.37 (m, 2H), 2.83 (t, J=5.2 Hz, 2H), 2.62 (s, 2H), 2.38-2.22 (m, 4H), 2.18-2.08 (m, 2H), 1.94-1.86 (m, 2H), 1.86-1.75 (m, 2H), 1.42 (d, J=4.2 Hz, 15H).

### Preparation of Intermediate NN: (7-tert-butyl-5-chloro-furo[3,2-b]pyridin-2-yl)methanol

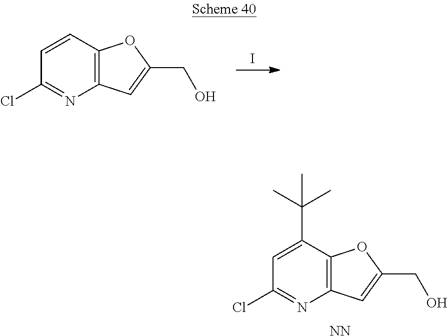

### Step I: (7-tert-butyl-5-chloro-furo[3,2-b]pyridin-2-yl)methanol

To a mixture of (5-chlorofuro[3,2-b]pyridin-2-yl)methanol (2.78 g, 15.1 mmol), 2,2-dimethyvlpropanoic acid (6.19 g, 3.48 mL, 60.6 mmol), water (13.9 mL), TFA (2.59 g, 1.75 mL, 22.7 mmol) and acetonitrile (28 mL) was added siver nitrate (1.29 g, 7.57 mmol). The reaction mixture was heated at 80° C. in the dark. A solution of ammonium persulfate (5.75 g, 27.3 mmol) in water (14 mL) was added dropwise to the reaction mixture and it was stirred at 80° C. for 30 minutes. Another portion of ammonium persulfate (5.75 g, 27.3 mmol) in water (14 mL) was added dropwise to the reaction mixture and it was stirred at 80° C. for an extra 30 minutes. The reaction mixture was cooled to room temperature and diluted with isopropyl acetate. The resulting bi-phasic mixture was allowed to cool down to room temperature, whereupon it was filtered over celite. The filtrate was cooled to 0° C. and basified to pH 9 by charging NH₄OH dropwise under strong stirring. The solution was stirred for 30 minutes and the layers were separated. The organic layer was washed with aqueous NaOH 1N and brine. The organic phase was concentrated under reduced pressure and the residue was purified by flash chromatography on silica gel eluting with 10-40% EtOAc: Hexanes affording the title compound (833 mg, 23% yield). ¹H NMR (400 MHz, DMSO-d6) δ 7.15 (s, 1H), 6.90-6.85 (m, 1H), 5.66 (t, J=6.0 Hz, 1H), 4.64 (dd, J=6.0, 0.9 Hz, 2H), 1.45 (s, 9H) ESI-MS m/z calc. 239.07, found 239.91 (M+1)⁺; Retention time: 1.43 minutes using method C.

### Preparation of Intermediate 00: methyl 6-(3,3-dimethyl-2-oxo-piperazin-1-yl)pyridine-2-carboxylate

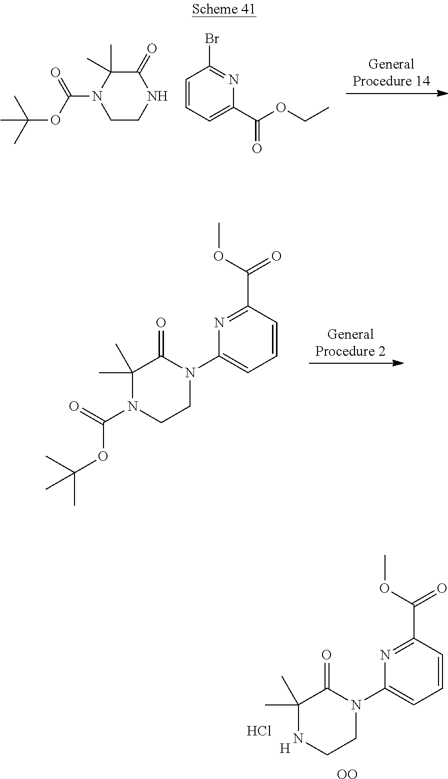

Under nitrogen, sodium hydride (53 mg, 1.3 mmol) was added to a stirred mixture of tert-butyl 2,2-dimethyl-3-oxo-piperazine-1-carboxylate (250 mg, 1.09 mmol) in DMF. The mixture was stirred for 20 min at rt and then ethyl 6-bromopyridine-2-carboxylate (378 mg, 1.64 mmol) is added. The reaction mixture was stirred for 3 h at 60° C. (in a seal tube, behind a protective shield) without success. The temperature was raised to 90° C. overnight, then 120° C. for 4 h. After cooling down to r.t., the solution was quenched with NH4Cl and extracted with EtOAc. Water was added along with EtOAc and the phases were separated. The organic phase was washed 2 other times with water and brine (1:1 mixture), dried over MgSO4, filtered and evaporated under reduced pressure. Crude product was dissolved in DCM and diazomethane (69 mg, 1.641 mmol) was added at r.t. and stirred for 15 min. Excess was quenched carefully with MeOH and evaporated under reduced pressure. Crude reaction mixture was purified using a 12 g silica gel column and a gradient of 0 to 40% EtOAc/Hexanes in to afford tert-butyl 4-(6-methoxycarbonyl-2-pyridyl)-2,2-dimethyl-3-oxo-piperazine-1-carboxylate (130 mg, 33%) 1H NMR (400 MHz, DMSO-d6) δ 8.08-7.99 (m, 2H), 7.90 (dd, J=7.1, 1.3 Hz, 1H), 4.08-4.01 (m. 2H), 3.89 (s, 3H), 3.77-3.68 (m, 2H), 1.67 (s, 6H), 1.46 (s, 9H). ESI-MS m/z calc. 363.1794, found 365.22 (M+1)+; Retention time: 0.83 minutes as a yellow solid. To tert-butyl 4-(6-methoxycarbonyl-2-pyridyl)-2,2-dimethyl-3-oxo-piperazine-1-carboxylate (130 mg, 0.358 mmol) was added a mixture of dioxane (2.6 mL) and MeOH (260 μL) while stirring until dissolution. HCl in dioxane (1 mL of 4 M, 4.0 mmol) was added and stirred for 18 h. Reaction mixture was evaporated under reduced pressure. A think yellow oil was obtained as a mixture of product containing methyl 6-(3,3-dimethyl-2-oxo-piperazin-1-yl)pyridine-2-carboxylate (Dihydrochloride salt) (120 mg, 0.3569 mmol, 99.75%) ESI-MS m/z calc. 263.12698, found 262.32 (M+1)+; Retention time: 0.15 minutes.

### Preparation of Intermediate PP: 3,3-dimethyl-1-(3-pyridyl)piperazin-2-one

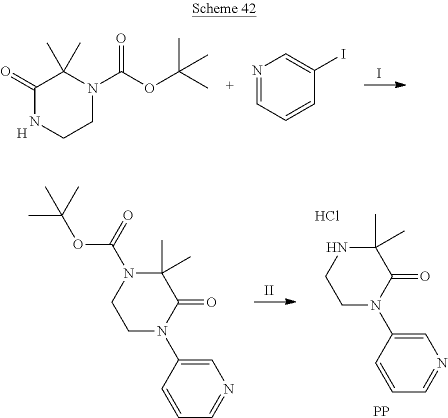

### Step I: tert-butyl 2,2-dimethyl-3-oxo-4-(3-pyridyl)piperazine-1-carboxylate

A pressure vessel was charged with tert-butyl 2,2-dimethyl-3-oxo-piperazine-1-carboxylate (1.36 g, 5.96 mmol), 3-iodopyridine (1.07 g. 5.22 mmol), K₃PO₄ (2.25 g, 10.6 mmol) and CuI (55 mg, 0.29 mmol). Toluene (11 mL) and N,N,N′,N′-tetramethylethane-1,2-diamine (80 μL, 0.5 mmol) are added, the tube was capped and transferred to a preheated (110° C.) oil bath and the reaction the mixture was stirred for 15 h then cooled to rt. Water and aq. sat. NH₄Cl were added to the solution and the aqueous phase was extracted 2 times with EtOAc. The combined organic phase was washed with aq. sat. NH₄Cl, brine dried over MgSO₄ and filtered. The filtrate was evaporated under reduced pressure. The residue was redisolved in EtOAc and the solution was filtered over silica and the filtrate evaporated under reduced pressure. The residue was then purified by flash chromatography on silica gel eluting with 50-100% EtOAc: Hexanes then 0-10% MeOH/EtOAc affording the title compound. tert-butyl 2,2-dimethyl-3-oxo-4-(3-pyridyl)piperazine-1-carboxylate (0.907 g, 57% yield) as an off-white solid. ESI-MS m/z calc. 305.17395, found 307.48 (M+1)+; Retention time: 0.54 minutes using method M.

### Step II: 3,3-dimethyl-1-(3-pyridyl)piperazin-2-one

A suspension of tert-butyl 2,2-dimethyl-3-oxo-4-(3-pyridyl)piperazine-1-carboxylate (906 mg, 2.97 mmol) in HCl in dioxane (7.5 mL of 4 M. 30.0 mmol) was sonicated then charged with MeOH (3.0 mL). The reaction mixture was sonicated again and stirred for 45 min at rt. The solid was filtered, washed with dioxane and dried in vacuo overnight affording the title compound 3,3-dimethyl-1-(3-pyridyl)piperazin-2-one (Dihydrochloride salt) (660 mg, 80% yield) as a white solid. ESI-MS m/z calc. 205.1215, found 206.5 (M+1)+; Retention time: 0.15 minutes using method J.

### Preparation of Intermediate SS: 2-(3,3-dimethylpiperazin-1-yl)-5-(1H-tetrazol-5-yl)pyrimidine

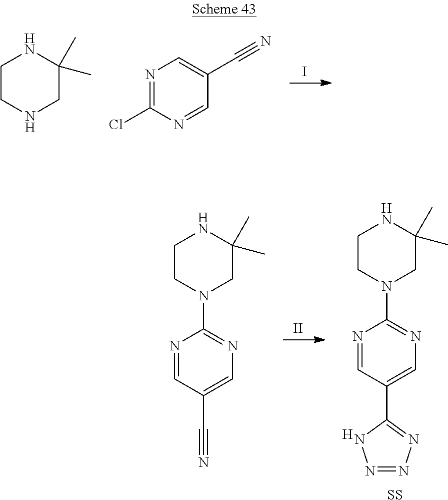

### Step I: 2-(3,3-dimethylpiperazin-1-yl)pyrimidine-5-carbonitrile

To a solution of 2,2-dimethylpiperazine (245 mg, 2.146 mmol) in dioxane (3.7 mL) was added 2-chloropyrimidine-5-carbonitrile (249.5 mg, 1.79 mmol) and the mixture is stirred overnight at room temperature. The mixture was diluted with ether and the solid is filtered off to afford 2-(3,3-dimethylpiperazin-1-yl)pyrimidine-5-carbonitrile (Hydrochloride salt) (458 mg, 100%)

### Step II: 2-(3,3-dimethylpiperazin-1-yl)-5-(1H-tetrazol-5-yl)pyrimidine (intermediate SS)

2-(3,3-dimethylpiperazin-1-yl)pyrimidine-5-carbonitrile (Hydrochloric Acid (1)) (63.7 mg, 0.251 mmol) was dissolved in DMF (1.1 mL), sodium azide (49 mg, 0.75 mmol) was added followed by N,N-diethylethanamine (Hydrochloric Acid (1)) (104 mg, 0.76 mmol). The reaction mixture was heated in the microwave for three hours at 130° C. The cooled reaction mixture was evaporated to dryness and the residue is purified by reverse phase chromatography, using 0 to 30% water in ACN gradient to afford 2-(3,3-dimethylpiperazin-1-yl)-5-(1H-tetrazol-5-yl)pyrimidine (Hydrochloride salt) (55.6 mg, 75%) 1H NMR (400 MHz, Methanol-d4) δ 8.93 (s, 2H), 4.09 (t, J=5.5 Hz, 2H), 3.93 (s. 2H), 3.32 (dd, J=6.2, 4.8 Hz, 2H), 1.39 (s, 6H). ESI-MS m/z calc. 260.14978, found 261.21 (M+1)+; Retention time: 1.08 minutes using Method K.

### Preparation of Intermediate TT: 6-(3,3-dimethylpiperazin-1-yl)pyridine-2-carbonitrile

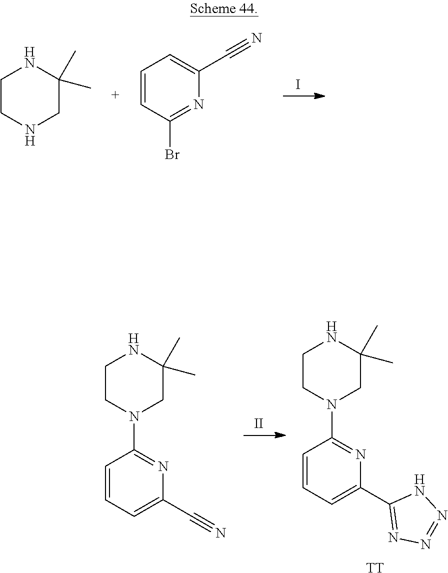

### Step I: 6-(3,3-dimethylpiperazin-1-yl)pyridine-2-carbonitrile

A mixture of 6-bromopyridine-2-carbonitrile (129.5 mg, 0.708 mmol), 2,2-dimethylpiperazine (97.0 mg, 0.849 mmol) and cesium carbonate (265.2 mg, 0.814 mmol) in NMP (1.3 mL) was heated to 100° C. for 16 hours. Water is then added (13 mL) and the mixture was extracted 3 times with 10 mL of ether. The organic layer was washed with water (3×5 mL) brine (5 mL) dried over Na₂SO₄, filtered and evaporated to dryness to afford 6-(3,3-dimethylpiperazin-1-yl)pyridine-2-carbonitrile (104 mg, 68%) 1H NMR (400 MHz, Chloroform-d) δ 7.49 (dd, J=8.8, 7.2 Hz, 1H), 6.93 (d, J=7.1 Hz, 1H), 6.79 (d, J=8.8 Hz, 1H), 3.57-3.53 (m, 2H), 3.03-3.00 (m, 2H), 1.18 (s, 6H). ESI-MS m/z calc. 216.1375, found 218.3 (M+1)⁺; Retention time: 0.74 minutes (method K).

### Step II

6-(3,3-dimethylpiperazin-1-yl)pyridine-2-carbonitrile (148.7 mg, 0.251 mmol) was dissolved in DMF (2.5 mL), sodium azide (134.2 mg, 2.064 mmol) was added followed by N,N-diethylethanamine (Hydrochloric Acid (1)) (284 mg, 2.06 mmol). The reaction mixture was heated in the microwave for three hours at 130° C. The cooled reaction mixture was evaporated to dryness and the residue was purified by reverse phase chromatography, using 0 to 30% water in ACN gradient to afford 3,3-dimethyl-1-[6-(1H-tetrazol-5-yl)-2-pyridyl]piperazine (155.3 mg, 0.5612 mmol, 81.61%) 1H NMR (400 MHz, Methanol-d4) δ 7.63 (dd, J=8.5, 7.4 Hz, 1H), 7.43 (d, J=7.4 Hz, 1H), 6.83 (d, J=8.5 Hz, 1H), 3.89 (t, J=5.4 Hz, 2H), 3.76 (s, 2H), 3.34-3.31 (m, 2H), 1.41 (s, 6H). ESI-MS m/z calc. 259.15454, found 260.23 (M+1)⁺; Retention time: 1.39 minutes (method K).

### Preparation of Intermediate UU: 5-(3,3-dimethylpiperazin-1-yl)pyrimidine

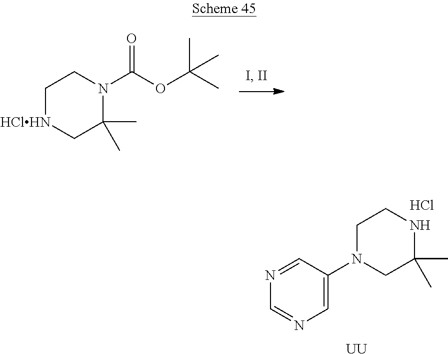

### Step I: tert-butyl 2,2-dimethyl-4-pyrimidin-5-yl-piperazine-1-carboxylate

A degassed mixture of tert-butyl 2,2-dimethylpiperazine-1-carboxylate hydrochloride (300 mg, 1.196 mmol), 5-bromopyrimidine (288 mg, 1.81 mmol), diacetoxypalladium(II) (29 mg, 0.129 mmol), [1-(2-diphenylphosphanyl-1-naphthyl)-2-naphthyl]-diphenyl-phosphane (75 mg, 0.120 mmol) and Cs₂CO₃ (980 mg, 3.0 mmol) in toluene was heated at 90° C. for 6 h. The mixture was then cooled to rt, diluted with DCM water and filtered. Layers were separated and the aqueous layer was back-extracted twice with DCM. The combined organic extracts were concentrated under reduced pressure. The crude product was purified by flash chromatography eluting with 0-100% EtOAc/hexanes to afford the title product tert-butyl 2,2-dimethyl-4-pyrimidin-5-yl-piperazine-1-carboxylate (214 mg, 61%) as an off-white solid. 1H NMR (400 MHz, dmso) a 8.45 (s, 1H), 8.31 (s, 2H), 3.74 (t, J=5.7 Hz, 2H), 3.52 (s, 2H), 3.36 (t, J=5.7 Hz, 2H), 1.41 (s, 9H), 1.33 (s, 6H). ESI-MS m/n calc. 292.1899, found 294.48 (M+1)⁺; Retention time: 0.64 minutes using method J.

### Step II: 5-(3,3-dimethylpiperazin-1-yl)pyrimidine (Intermediate UU)

A mixture of tert-butyl 2,2-dimethyl-4-pyrimidin-5-yl-piperazine-1-carboxylate (214 mg, 0.732 mmol) in HCl in dioxane (4M, 3.65 mL, 14.60 mmol) was stirred at rt for 2 h. The reaction mixture was concentrated under reduced pressure to afford the title 5-(3,3-dimethylpiperazin-1-yl)pyrimidine dihydrochloride salt (206 mg, quantitative yield), which was used in the subsequent step without further treatment. ESI-MS m/z calc. 192.1375, found 193.63 (M+1)⁺; Retention time: 0.57 minutes using method L.

### Preparation of Intermediate VV: ethyl 2-(8,8-dimethyl-5,6,7,8-tetrahydropyrido[3,4-d]pyrimidin-2-yl)acetate

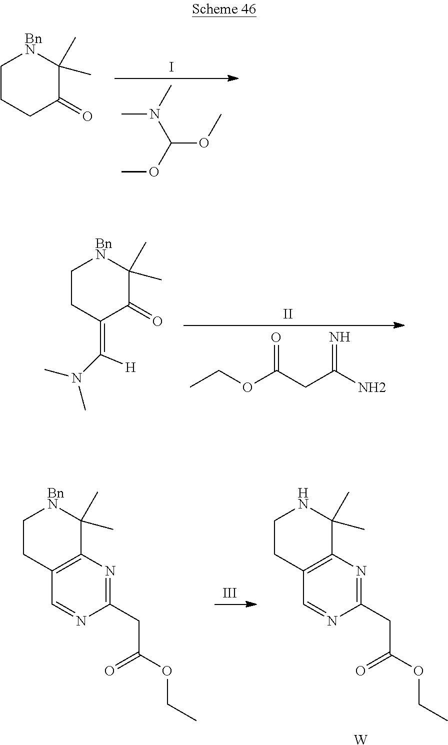

### Step I: (4E)-1-benzyl-4-(dimethylaminomethylene)-2,2-dimethyl-piperidin-3-one

The solution of 1-benzyl-2,2-dimethyl-piperidin-3-one (1.55 g, 7.13 mmol) in 1,1-dimethoxy-N,N-dimethyl-methanamine (4.7 mL, 35 mmol) was heated at 100° C. under nitrogen for 18 h. The volatiles were removed under reduced pressure to afford the title compound (1.55 g, 95%) as a brown solid, which was used in the subsequent step without further treatment. ESI-MS m/z calc. 272.18887, found 273.37 (M+1)⁺; Retention time: 0.74 minutes using method L.

### Step II: ethyl 2-(7-benzyl-8,8-dimethyl-5,6-dihydropyrido[3,4-d]pyrimidin-2-yl)acetate

To a solution of (4E)-1-benzyl-4-(dimethylaminomethylene)-2,2-dimethyl-piperidin-3-one (200 mg, 0.734 mmol) in EtOH (4 mL) were added ethyl 3-amino-3-imino-propanoate (191 mg, 1.47 mmol) and sodium ethoxide (410 μL of 2.67 M, 1.1 mmol). The mixture was heated at 80° C. for 12 h. After cooling to rt, the mixture was acidified with 2M HCl to pH 3, then basified with sat. NaHCO₃. The mixture was diluted with water and extracted with EtOAc (2×20 mL). The combined organic extracts were washed with water and brine, filtered and concentrated to dryness. The crude product was purified by flash chromatography eluting with 0-30% EtOAc/hexanes to afford the title product ethyl 2-(7-benzyl-8,8-dimethyl-5,6-dihydropyrido[3,4-d]pyrimidin-2-yl)acetate (140 mg, 56%) as a white solid. ¹H NMR (400 MHz, Chloroform-d) δ 7.83 (s, 1H), 7.44 (d, J=7.7 Hz, 2H), 7.35 (t, J=7.4 Hz, 2H), 7.26 (m, 1H), 6.14 (s, 2H), 4.35 (q. J=7.1 Hz, 2H), 3.75 (s, 2H), 2.69 (t, J=5.7 Hz, 2H), 2.60 (t, J=5.6 Hz, 2H), 1.52 (s, 6H), 1.39 (t, J=7.1 Hz, 3H). ESI-MS m/z calc. 339.19467, found 340.37 (M+1)⁺; Retention time: 0.89 minutes using method C.

### Step III: ethyl 2-(8,8-dimethyl-6,7-dihydro-5H-pyrido[3,4-d]pyrimidin-2-yl)acetate (Intermediate VV)

To a solution ethyl 2-(7-benzyl-8,8-dimethyl-5,6-dihydropyrido[3,4-d]pyrimidin-2-yl)acetate (140 mg, 0.412 mmol) in EtOH (4 mL) and THF (1 mL) was added 20% Pd(OH)₂/C (35 mg, 0.050 mmol). The nitrogen atmosphere was evacuated under reduced pressure and re-filled with hydrogen 3 times, and the reaction mixture was stirred under a hydrogen atmosphere (normal pressure) at rt for 2 h. The reaction mixture was filtered and the volatiles were removed under reduced pressure to afford the title compound ethyl 2-(8,8-dimethyl-6,7-dihydro-5H-pyrido[3,4-d]pyrimidin-2-yl)acetate (102 mg, quantitative yield) as a white solid, which was used in the subsequent step without further treatment. 1H NMR (400 MHz, Chloroform-d) δ 7.80 (d, J=0.9 Hz, 1H), 6.12 (s, 2H), 4.30 (q, J=7.1 Hz, 2H), 3.10 (t, J=5.9 Hz, 2H), 2.70 (td, J=6.0, 0.9 Hz, 2H), 1.44 (s, 6H), 1.35 (t, J=7.1 Hz, 3H). ESI-MS m/z calc. 249.14772, found 250.32 (M+1)⁺; Retention time: 0.63 minutes using method C.

### Preparation of Intermediate WW: 6-(3,3-dimethylpiperazin-1-yl)-5-methyl-pyridine-2-carboxylate

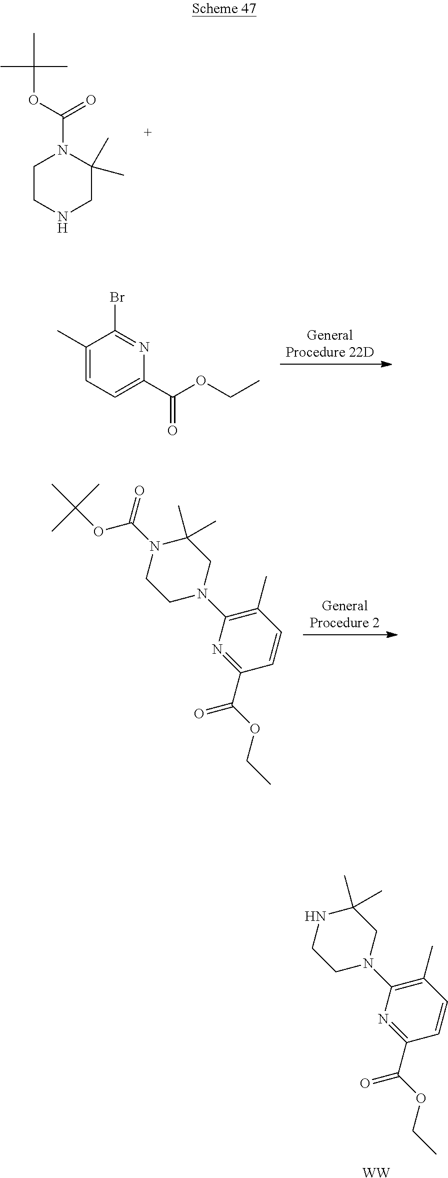

### Step 1

A mixture of tert-butyl 2,2-dimethylpiperazine-1-carboxylate (HCl salt) (420 mg, 1.67 mmol), ethyl 6-bromo-5-methyl-pyridine-2-carboxylate (613.1 mg, 2.51 mmol) and cesium carbonate (1.67 g, 5.11 mmol) in NMP (8.4 mL) was heated to 110° C. for 16 h. An extra 0.5 equivalent of ethyl 6-bromo-5-methyl-pyridine-2-carboxylate was added and, the reaction was stirred for two days at 110° C. The mixture was cooled to RT and water was added. The aqueous phase was extracted twice with EtOAc. The combined organic extracts were washed twice with water, then brine, dried over sodium sulfate, filtered, and the solvent was removed under reduced pressure. The residue was then purified by silica gel chromatography eluting with 0 to 40% EtOAc/Hexanes to afford the N-Boc piperazine intermediate.

### Step 2

The N-Boc intermediate was dissolved in dioxane (8.4 mL) and treated with an HCl solution in dioxane (1.68 mL of 4 M, 6.7 mmol) and the mixture was stirred at rt overnight. After one day, an extra 4 equiv. HCl in dioxane (4M) was added and. The volatiles were removed under reduced pressure to afford crude intermediate WW, ethyl 6-(3,3-dimethylpiperazin-1-yl)-5-methyl-pyridine-2-carboxylate (23 mg, 5%). ESI-MS m/z calc. 277.17902, found 278.31 (M+1)⁺; Retention time: 0.4 minutes using method J.

### Preparation of Intermediate XX: methyl 6-(3,3-dimethyl-2-oxo-piperazin-1-yl)pyridine-3-carboxylate

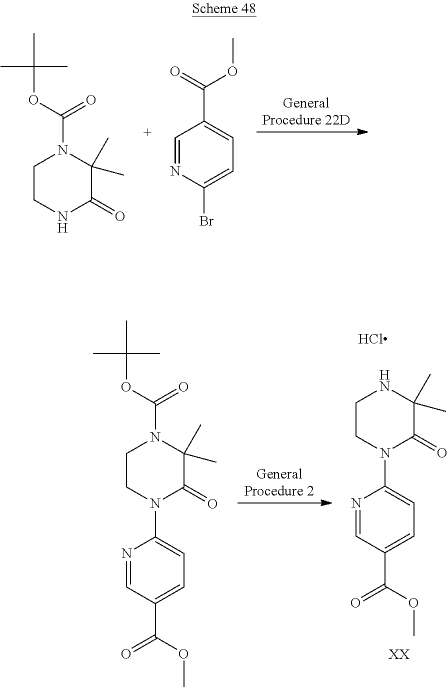

### Step 1: tert-butyl 4-(5-methoxycarbonyl-2-pyridyl)-2,2-dimethyl-3-oxo-piperazine-1-carboxylate

Under nitrogen, sodium hydride (40 mg, 1.0 mmol) was added to a stirred mixture of tert-butyl 2,2-dimethyl-3-oxo-piperazine-1-carboxylate (200 mg, 0.87 mmol) in DMF. The mixture was stirred at r.t. for and, methyl 6-bromopyridine-3-carboxylate (281 mg, 1.30 mmol) was then added. The reaction mixture was stirred for 2 h at 60° C., then ON and was quenched by adding water. The resulting solid was stirred for 1 h and then filtered on Buchner and washed with water followed by heptane to give tert-butyl 4-(5-methoxycarbonyl-2-pyridyl)-2,2-dimethyl-3-oxo-piperazine-1-carboxylate (73 mg, 19%) ESI-MS m/z calc. 363.1794, found 365.27 (M+1)+; Retention time: 0.96 using Method J.

### Step2: Intermediate XX

Tert-butyl 4-(5-methoxycarbonyl-2-pyridyl)-2,2-dimethyl-3-oxo-piperazne-1-carboxylate (73 mg, 0.166 mmol) was dissolved in DCM (2 mL) and TFA (2.5 mL, 32. 5 mmol) was then added. The reaction mixture was stirred for 1 h at r.t. After complete deprotection, volatiles were evaporated under reduced pressure. The solid was redissolved in DCM and treated with aqueous saturated solution of NaHCO₃. The layers were separated and concentrated to afford the intermediate XX, methyl 6-(3,3-dimethyl-2-oxo-piperazin-1-yl)pyridine-3-carboxylate (43.6 mg, 100%) ESI-MS m/z calc. 263.12698, found 264.21 (M+1)+; Retention time: 0.22 minutes using Method J.

### Preparation of intermediate YY

Mixture regioisomers 1-[2-[(4-methoxyphenyl)methyl]pyrazol-3-yl]-3,3-dimethyl-piperazin-2-one and 1-[1-[(4-methoxyphenyl)methyl]pyrazol-3-yl]-3,3-dimethyl piperazin-2-one (Hydrochloride salts)

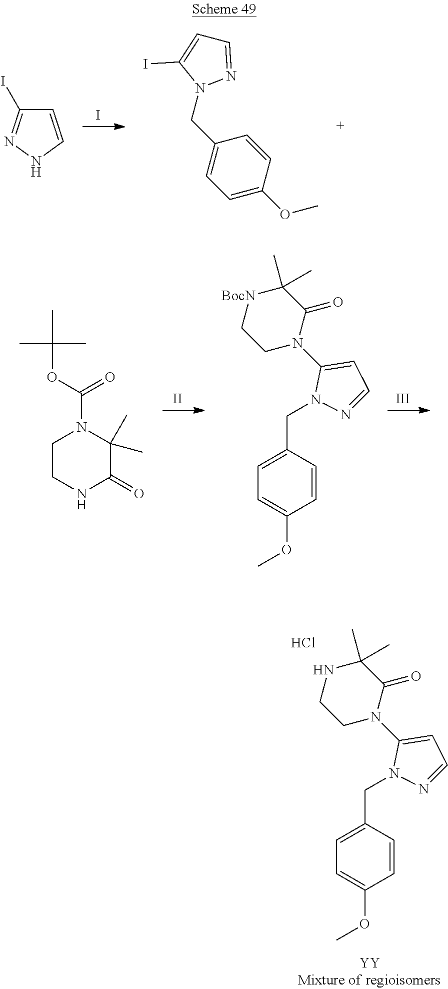

### Step I: 5-iodo-1-[(4-methoxyphenyl)methyl]pyrazole and 3-iodo-1-[(4-methoxyphenyl)methyl]pyrazole (Mixture of Regioisomers)

1-(chloromethyl)-4-methoxy-benzene (2.10 mL, 15.5 mmol) was added to a stirred mixture of 3-iodo-1H-pyrazole (2.01 g. 10.4 mmol) and Cs₂CO₃ (6.70 g, 20.6 mmol) in DMF (30.0 mL). The reaction mixture was stirred at 60° C. for 2 hours and then, cooled to room temperature. Water (100 mL) was added and aqueous was extracted with EtOAc (3×50 mL). Combined organic extracts were washed with aqueous saturated NH₄C (25 mL) solution, brine (25 mL) and dried over MgSO₄. The mixture was filtered and concentrated to afford 4.06 g of crude material. The residue was adsorbed on silica using DCM and purified by silica gel chromatography to afford 3-iodo-1-[(4-methoxyphenyl)methyl]pyrazole and 5-iodo-1-[(4-methoxyphenyl)methyl]pyrazole (3.18 g, 98%) as a white solid as a ca. 5:1 mixture of regioisomers. Major Regioisomer: ¹H NMR (400 MHz, CDCl₃) δ 7.23-7.15 (m, 3H), 7.12 (d, J=2.3 Hz, 1H), 6.92-6.85 (m, 2H), 6.40 (d, J=2.3 Hz, 1H), 5.24 (s, 2H): ESI-MS m/z calc. 313.9916, found 314.98 (M+1)+; Retention time: 0.93 minutes Using Method J.

### Step II: tert-butyl 4-[2-[(4-methoxyphenyl)methyl]pyrazol-3-yl]-2,2-dimethyl-3-oxo-piperazine-1-carboxylate and tert-butyl 4-[1-[(4-methoxyphenyl)methyl]pyrazol-3-yl]-2,2-dimethyl-3-oxo-piperazine-1-carboxylate (Mixture Regioisomers)

A mixture of tert-butyl 2,2-dimethyl-3-oxo-piperazine-1-carboxylate (915 mg, 4.01 mmol), regioisomers mix of 5-iodo-1-[(4-methoxyphenyl)methyl]pyrazole/3-iodo-1-[(4-methoxyphenyl)methyl]pyrazole (1.51 g, 4.81 mmol), iodocopper (381.7 mg, 2.00 mmol), N,N′-dimethylethane-1,2-diamine (353.3 mg, 426.7 μL, 4.01 mmol) and K₃PO₄ (1.702 g, 8.02 mmol) in DMF (18.3 mL) was heated at 120° C. for 3.5 hours. The reaction mixture was cooled to r.t. and filtered to remove the copper salts. The filtrate was diluted with water and aqueous was extracted twice with ethyl acetate. Organic extracts were washed with water, followed with brine, dried over Na₂SO₄, filtered and concentrated under reduced pressure. The recovered crude compound was purified by silica gel chromatography to give a mixture of the tert-butyl 4-[2-[(4-methoxyphenyl)methyl]pyrazol-3-yl]-2,2-dimethyl-3-oxo-piperazine-1-carboxylate and the tert-butyl 4-[1-[(4-methoxyphenyl)methyl]pyrazol-3-yl]-2,2-dimethyl-3-oxo-piperazine-1-carboxylate (1.603 g, 96%). Major regioisomer: ¹H NMR (400 MHz, DMSO-d6) δ 7.70 (d, J=2.3 Hz, 1H), 7.22-7.11 (m, 2H), 6.91-6.81 (m, 2H), 6.61 (d, J=2.3 Hz, 1H), 5.14 (s, 2H), 3.86-3.79 (m, 2H), 3.69 (s, 3H), 3.66-3.58 (m, 2H), 1.58 (s, 6H), 1.40 (s, 9H). ESI-MS m/z calc. 414.2267, found 416.38 (M+1)+.

### Step III (Intermediate YY): 1-[2-[(4-methoxyphenyl)methyl]pyrazol-3-yl]-3,3-dimethyl-piperazin-2-one and 1-[1-[(4-methoxyphenyl)methyl]pyrazol-3-yl]-3,3-dimethyl-piperazin-2-one (Hydrochloride Salts)

A HCl solution in dioxane (4.84 mL of 4 M, 19.3 mmol) was added to a stirred solution of the mixture of regioisomers tert-butyl 4-[2-[(4-methoxyphenyl)methyl]pyrazol-3-yl]-2,2-dimethyl-3-oxo-piperazine-1-carboxylate/tert-butyl 4-[1-[(4-methoxyphenyl)methyl]pyrazol-3-yl]-2,2-dimethyl-3-oxo-piperazine-1-carboxylate (1.60 g, 3.87 mmol) in DCM (16 mL). The reaction mixture was stirred 24 h at room temperature and then, solvents were removed by evaporation in vacuo. The recovered gummy residue was dissolved in DCM and sonicated. Diethyl ether was added dropwise until full precipitation of the desired product in the sonicator bath. The mixture was filtered. The recovered white solid was washed with diethyl ether and dried under vacuum to give a mixture of regioisomers 1-[2-[(4-methoxyphenyl)methyl]pyrazol-3-yl]-3,3-dimethyl-piperazin-2-one and 1-[1-[(4-methoxyphenyl)methyl]pyrazol-3-yl]-3,3-dimethyl-piperazin-2-one (1.153 g, 85%) as hydrochloride salt. Major isomer: ¹H NMR (400 MHz, DMSO-d6) δ 10.07 (broad s, 1H), 7.79 (d, J=2.3 Hz, 1H), 7.23-7.08 (m, 2H), 6.93-6.83 (m, 2H), 6.64 (d, J=2.3 Hz, 1H), 5.18 (s, 2H), 4.04 (t, J=5.8 Hz, 2H), 3.71 (s, 3H), 3.65-3.50 (m, 2H), 3.42-3.32 (m, 1H), 1.58 (s, 6H). ESI-MS m/z calc. 314.1743, found 315.78 (M+1)+; Retention time: 0.34 using Method J.

### Preparation of Intermediate ZZ: 1-[1-[(4-methoxyphenyl)methyl]pyrazol-4-yl]-3,3-dimethyl-piperazin-2-one (Hydrochloride Salt)

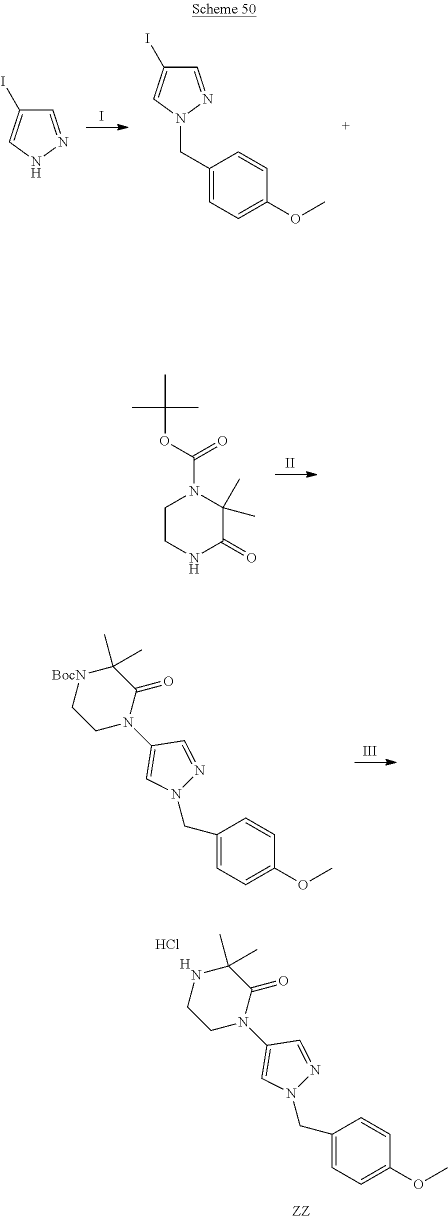

### Step I: 4-iodo-1-[(4-methoxyphenyl)methyl]pyrazole

A mixture of 4-iodo-1H-pyrazole (26 g, 134.0 mmol), 1-(chloromethyl)-4-methoxy-benzene (30.1 g, 192.4 mmol), cesium carbonate (64.7 g. 198.6 mmol) in DMF (267 mL) was heated at 60° C. overnight. The reaction mixture was diluted with 250 mL water and extracted with 2×250 mL ethyl acetate. The combined organic layers were washed with brine then dried over Na₂SO₄. The mixture was filtered then evaporated and the residue pre-adsorbed on silica gel. Purified by silica gel plug filtration using 0-10% EtOAc/Hexanes as eluent.

### Step II: 1-[1-[(4-methoxyphenyl)methyl]pyrazol-4-yl]-3,3-dimethyl-piperazin-2-one

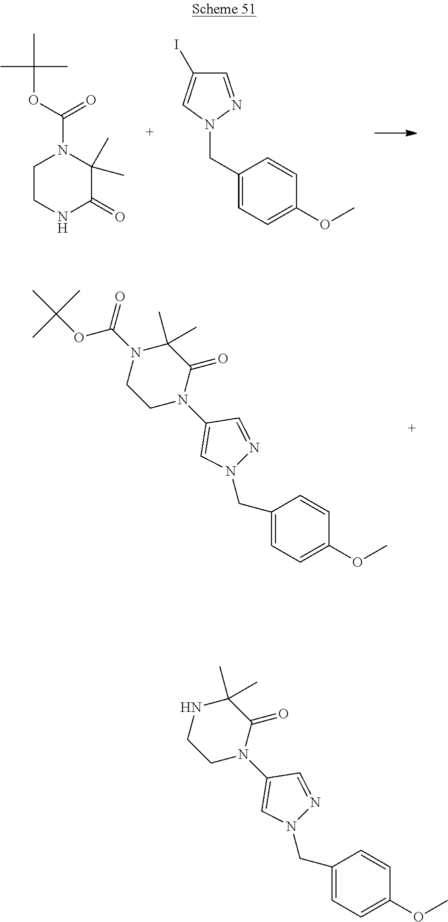 [SMILES: CC(C)(C)OC(=O)N1CCNC(=O)C1(C)C ; COc1ccc(Cn2cc(I)cn2)cc1 ; COc1ccc(Cn2cc(N3CCN(C(=O)OC(C)(C)C)C(C)(C)C3=O)cn2)cc1 ; COc1ccc(Cn2cc(N3CCNC(C)(C)C3=O)cn2)cc1]

A mixture of tert-butyl 2,2-dimethyl-3-oxo-piperazine-1-carboxylate (3.42 g, 15 mmol), 4-iodo-1-[(4-methoxyphenyl)methyl]pyrazole (5.18 g, 16.5 mmol), iodocopper (1.43 g, 7.50 mmol), N,N′-dimethylethane-1,2-diamine (1.322 g, 1.60 mL, 15.00 mmol), K₃PO₄ (6.368 g, 30.00 mmol) in DMF (78.8 mL) was heated at 120 C for 4 h. The reaction mixture is diluted with water (150 mL) and extracted (2×) with EtOAc (2×150 mL). The combined organics are washed with water (150 mL) and brine (100 mL), evaporated in vacuo and purified by flash column chromatography.

To the material obtained above, in dioxane (34.2 mL) and MeOH (1.7 mL) is added HCl (18.8 mL, 4 M, 75.0 mmol). The reaction is allowed to stir at RT for 4 h. The solvent is removed in vacuo to give a gummy solid. This is triturated with DCM (200 mL) to give a white suspension which is suction-filtered to give a white powder. 1-[1-[(4-methoxyphenyl)methyl]pyrazol-4-yl]-3,3-dimethyl-piperazin-2-one (Hydrochloride salt) (5.41 g, 103%) ¹H NMR (400 MHz, DMSO-d6) δ 10.18 (s, 2H), 8.13 (d, J=0.8 Hz, 1H), 7.66 (d, J=0.8 Hz, 1H), 7.24-7.11 (m, 2H), 6.93-6.75 (m, 2H), 5.19 (s, 2H), 3.93-3.83 (m, 2H), 3.69 (s, 3H), 3.58-3.45 (m, 2H), 1.55 (s, 6H).

### Synthesis of Intermediate BBB: Example 62

### Preparation of Intermediate CCC: (7-(tert-butyl)-5-(4,4-dichlorocyclohexyl)furo[3,2-b]pyridin-2-yl)(2,2-dimethylpiperazin-1-yl)methanone

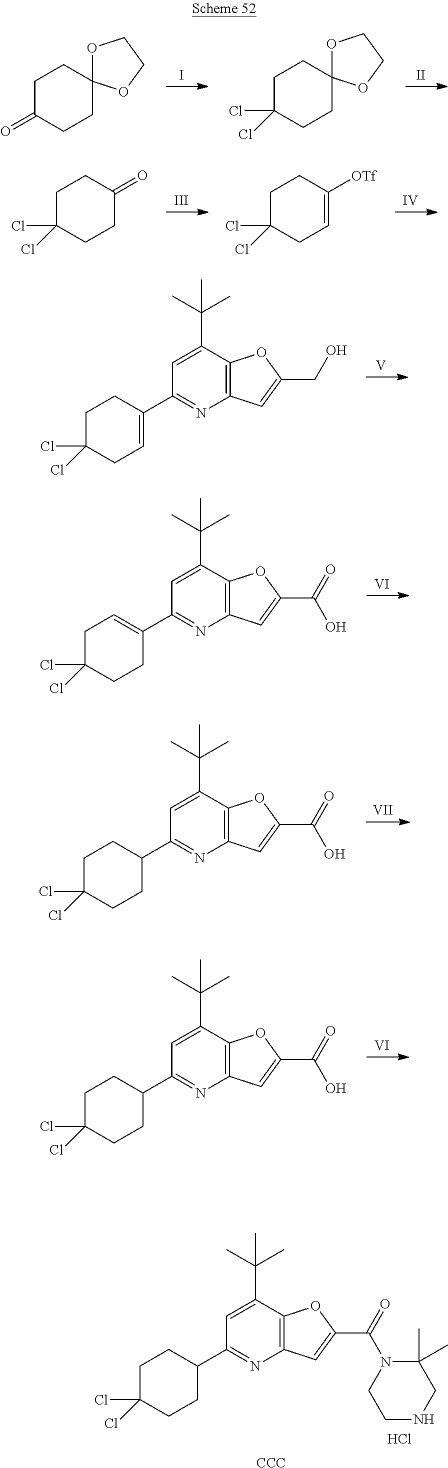

### Step I: 8,8-dichloro-1,4-dioxaspiro[4.5]decane

To a mixture of activated 4A molecular sieves and hydrazine hydrate (62.3 mL, 1.28 mol) in dry MeOH (267 mL) was added 1,4-dioxaspiro[4.5]decan-8-one (10 g, 64.03 mmol) in MeOH (160 mL) dropwise and the mixture was stirred at room temperature for 2 h. The suspension was then filtered on celite, washed with MeOH and the volatiles were removed under reduced pressure. In a separate flask, triethyl amine (22.7 g, 31.2 mL, 224.1 mmol) was added dropwise to a suspension of CuCl₂ (60.26 g, 448.2 mmol) in MeOH (250.0 mL) at 0° C. The mixture was stirred for 30 minutes and for an additional 2 h at room temperature. The mixture was cooled back to 0° C. and a solution of the hydrazone prepared earlier in MeOH (200 mL) and the solution stirred at room temperature for 16 h. The solution is diluted with EtOAc and 1N HCl was added. The phases were separated and the aqueous phase was extracted twice with EtOAc. The combined organic phase was washed with brine, dried over MgSO4, filtered and the filtrate evaporated under reduced pressure affording the title compound as a brown solid which was used in the subsequent step without further purification (13.5 g, 63.95 mmol, 99.85%) ¹H NMR (400 MHz, dmso) δ 3.89 (s, 4H), 2.46-2.39 (m, 4H), 1.79-1.73 (m, 4H).

### Step II: 4,4-dichlorocydohexanone

To a solution of 8,8-dichloro-1,4-dioxaspiro[4.5]decane (13.4 g, 63.5 mmol) in acetone (158.7 mL) was added HCl (222.2 mL of 2 M. 444.4 mmol) and the solution was stirred at room temperature for 16 h. Acetone was removed under reduced pressure and the aq. phase was extracted with EtOAc three times. The combined organic phase was washed with brine, dried over MgSO4, filtered and the filtrate evaporated under reduced pressure. The residue was purified by silica gel chromatography affording the title compound as a white solid. 4,4-dichlorocyclohexanone (3.8 g, 22.75 mmol, 35.85%) ¹H NMR (400 MHz, dmso) δ 2.74 (t, J=6.7 Hz, 4H), 2.47 (t, J=6.7 Hz, 4H).

### Step III: (4,4-dichlorocyclohexen-1-yl) trifluoromethanesulfonate

To a solution of diisopropylamine (726.9 mg, 1.007 mL, 7.184 mmol) in THF (10.65 mL) at 0° C. was added n-butyl lithium in hexanes (2.874 mL of 2.5 M, 7.184 mmol) and the resulting solution was stirred at this temperature for 15 minutes. The solution was cooled to −78° C. and 4,4-dichlorocyclohexanone (1000 mg, 5.99 mmol) in THF (15.97 mL) was added dropwise. The solution was stirred at −78° C. for 45 minutes before a solution of Comins' Reagent (2.59 g, 6.59 mmol) in THF (10.65 mL) was added dropwise over 15 minutes. The solution was stirred cold for 1 h, diluted with ammonium chloride and warmed to room temperature. The aqueous phase was extracted twice with EtOAc, the combined organic phase was washed with 1M NaOH and brine, dried over MgSO4, filtered and the filtrate evaporated under reduced pressure. The residue was purified by silica gel chromatography on a 50 g column eluting with 0 to 15% EtOAc/Hexanes affording the title compound (4,4-dichlorocyclohexen-1-yl) trifluoromethanesulfonate (1290 mg, 72%). ¹H NMR (400 MHz, Chloroform-d) δ 5.68-5.57 (m, 1H), 3.23-3.08 (m, 2H), 2.65-2.60 (m, 2H), 2.59-2.54 (m, 2H).

### Step IV: [7-tert-butyl-5-(4,4-dichlorocyclohexen-1-yl)furo[3,2-b]pyridin-2-yl]methanol

To a solution of (4,4-dichlorocyclohexen-1-yl) trifluoromethanesulfonate (277.0 mg, 0.926 mmol) and bis(pinacol)diboron (245.0 mg, 0.965 mmol) in dioxane (2.3 mL) was added potassium acetate (181.8 mg, 1.85 mmol) and the reaction mixture is degassed with nitrogen for 5 minutes. PdCl₂-dppf (84.22 mg, 0.115 mmol) was added and the solution was stirred at 80° C. for 2 h. The solution was cooled to room temperature and PdCl₂-dppf (84.2 mg, 0.115 mmol), Na₂CO₃ (1.75 mL of 2 M, 3.49 mmol) and (7-tert-butyl-5-chloro-furo[3,2-b]pyridin-2-yl)methanol (185 mg, 0.772 mmol) were added and the solution stirred at 80° C. for 2 h. The crude reaction mixture was purified by silica gel chromatography on a 25 g column eluting with 0 to 50% EtOAc/Hexanes (18 CV) affording [7-tert-butyl-5-(4,4-dichlorocyclohexen-1-yl)furo[3,2-b]pyridin-2-yl]methanol (165 mg, 0.466 mmol. 60.35%) ¹H NMR (400 MHz, Chloroform-d) δ 7.18 (s, 1H), 6.82 (s, 1H), 6.34 (s, OH), 4.78 (d, J=0.9 Hz, 2H), 3.28-3.15 (m, 2H), 2.96-2.79 (m, 1H), 2.57 (t, J=6.2 Hz, 2H), 1.46 (s, 9H), 1.31-1.24 (m, 1H). ESI-MS m/z calc. 353.09494, found 354.32 (M+1)+; Rt: 1.85 minutes using Method C.

### Step V: 7-tert-butyl-5-(4,4-dichlorocylohexen-1-yl)furo[3,2-b]pyridine-2-carboxylic acid

To a suspension of [7-tert-butyl-5-(4,4-dichlorocyclohexen-1-yl)furo[3,2-b]pyridin-2-yl]methanol (210 mg, 0.593 mmol) in MeCN (4.2 mL) was added 4-methyl-4-oxido-morpholin-4-ium hydrate (909.0 mg, 5.93 mmol) at room temperature. To the resulting solution was added TPAP (10.7 mg, 0.0304 mmol) at room temperature. After 2 h iPrOH is added (456 μL, 5.95 mmol) and the mixture allowed to stir overnight. Water (5 mL) was added and the mixture concentrated to remove volatiles then acidified with 2N HCl (2 mL, pH [3.0-4.0]). The resulting solid was collected by filtration, washed with H₂O and air dried overnight. 7-tert-butyl-5-(4,4-dichlorocyclohexen-1-yl)furo[3,2-b]pyridine-2-carboxylic acid (150 mg, 68.71%) ¹H NMR (400 MHz, DMSO-d6) δ 7.65 (s, 1H), 7.48 (s, 1H), 6.55 (t, J=1.7 Hz, 1H), 3.27-3.21 (m, 2H), 2.89-2.78 (m, 2H), 2.64-2.53 (m, 2H), 1.46 (s, 9H). ESI-MS m/z calc. 367.0742, found 368.27 (M+1)+; Rt: 1.96 minutes using Method C.

### Step VI: 7-tert-butyl-5-(4,4-dichlorocyclobexyl)furo[3,2-b]pyridine-2-carboxylic acid

A solution of 7-tert-butyl-5-(4,4-dichlorocyclohexen-1-yl)furo[3,2-b]pyridine-2-carboxylic acid (52 mg, 0.1412 mmol) in MeOH (520.0 μL) and EtOAc (1.0 mL) was prepared and added Pd on carbon (14.97 mg, 0.01407 mmol). The reaction vessel was then equipped with an H2-balloon (1 atm) and the reaction mixture is stirred for 6 h. Since there was concomitant over-hydrogenation, most probably of the furan moiety, the hydrogen balloon was removed at the end of the day, and placed back the morning after. After an additional 4 d, the reaction was complete by LCMS. ESI-MS m/z calc. 369.08984, found 370.28 (M+1)+. Rt: 3.75 minutes using Method A. ¹H NMR (400 MHz, Chloroform-d) δ 11.58 (s, 1H), 7.79 (s, 1H), 7.18 (s, 1H), 3.20-3.02 (m, 1H), 2.72-2.58 (m, 2H), 2.47-2.28 (m, 2H), 2.17-2.07 (m, 4H), 1.53 (s, 9H).

### Step VII: [7-tert-butyl-5-(4,4-dichlorocyclohexyl)furo[3,2-b]pyridin-2-yl]-(2,2-dimethylpiperazin-1-yl)methanone hydrochloride salt

To a solution of tert-butyl 3,3-dimethylpiperazine-1-carboxylate (152.4 mg, 0.711 mmol) in dry DMF (2.5 mL) was added 7-tert-butyl-5-(4,4-dichlorocyclohexyl)furo[3,2-b]pyridine-2-carboxylic acid (240 mg, 0.6482 mmol), DIPEA (212.3 mg, 286.1 μL, 1.64 mmol) and HATU (271.6 mg, 0.7143 mmol). After 30 min, H₂O (20 mL) was added. The mixture was extracted with EtOAc (×3), dried (MgSO₄), and the solvent was removed under reduced pressure to afford tert-butyl 4-[7-tert-butyl-5-(4,4-dichlorocyclohexyl)furo[3,2-b]pyridine-2-carbonyl]-3,3-dimethyl-piperazine-1-carboxylate (235 mg, 64%).

To the N-Boc intermediate in dioxane (4.94 mL) was added HCl in dioxane (810.2 μL 4 M, 3.24 mmol). The mixture was stirred for 24 h, and MeOH (0.05 mL) was added and stirring continued for 3 h. The solvent was removed in vacuo to give 7-tert-butyl-5-(4,4-dichlorocyclohexyl)furo[3,2-b]pyridin-2-yl]-(2,2-dimethylpiperazin-1-yl)methanone (Hydrochloride salt) (210 mg, 101%). ESI-MS m/z calc. 465.19498, found 466.3 (M+1)+; Rt: 1.27 minutes using Method C.

### Preparation of Intermediate DDD: 4,4,5,5-tetramethyl-2-(spiro[3,3]hept-1-en-2-yl)-1,3,2-dioxaborolane

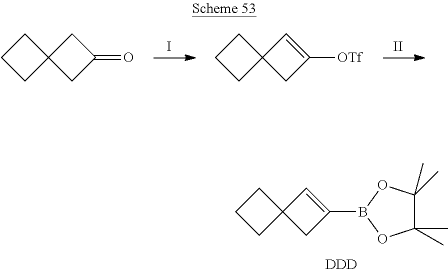

### Step 1: spiro[3,3]hept-1-en-2-yl trifluoromethanesulfonate

Spiro[3.3]heptan-2-one (25 g, 227.0 mmol) in THF (375.0 mL) was added dropwise to a solution of LiHMDS (295.1 mL of 1 M, 295.1 mmol) in THF at −78 OC under a nitrogen atmosphere. The solution is stirred at 0° C. for 2 h. After cooling to −78 OC a solution of PhN(SO₂CF₃)₂ (121.6 g, 340.5 mmol) in THF (375.0 mL) was added dropwise (the mixture becomes milky and thicker upon addition) and was stirred at −78° C. for 2 h before warming to RT. Ether was added to the mixture is washed (H₂O, brine), dried (Na₂SO₄) and evaporated to yield a brown residue that was filtered through a plug of silica using 10% EtOAc in hexanes as the eluent. The brown oil obtain after evaporation was taken in a minimum of hexanes and purified by silica gel chromatography using a gradient of 0 to 10% EtOAc in hexanes to obtain a clear oil (46 g).

### Step II: 4,4,5,5-tetramethyl-2-(spiro[3,3]hept-1-en-2-yl)-1,3,2-dioxaborolane

Cyclopentyl(diphenyl)phosphane;dichloropalladium;iron (6.947 g, 9.495 mmol) was added to spiro[3.3]hept-2-en-2-yl trifluoromethanesulfonate (46 g, 189.9 mmol), potassium acetate (74.55 g, 759.6 mmol) and 4,4,5,5-tetramethyl-2-(4,4,5,5-tetramethyl-1,3,2-dioxaborolan-2-yl)-1,3,2-dioxaborolane (96.45 g, 379.8 mmol) in dioxane (920.0 mL) and the mixture was heated to 75° C. under nitrogen for 4 h. The solvent was evaporated, and the brown residue was taken in Hexanes and filtrated on a silica gel plug using 10% EtOAc in Hexanes as the eluent. The filtrate was evaporated and dissolved in a minimum amount of hexane. On standing, a solid precipitated which was filtered, and remaining solution was purified by silica gel chromatography (0 to 15% EtOAc in hexanes to afford the title compound as a colorless oil (24.7 g). ¹H NMR (400 MHz, Chloroform-d) δ 6.94-6.89 (m, 1H), 2.57-2.50 (m, 2H), 2.18-2.00 (m, 4H), 1.90-1.67 (m, 2H), 1.26-1.19 (m, 12H).

### Preparation of Intermediate FFF: Example 63

### Preparation of Intermediate GGG: ethyl 2-(2,2-dimethylpiperidin-4-yl)acetate hydrochloride salt

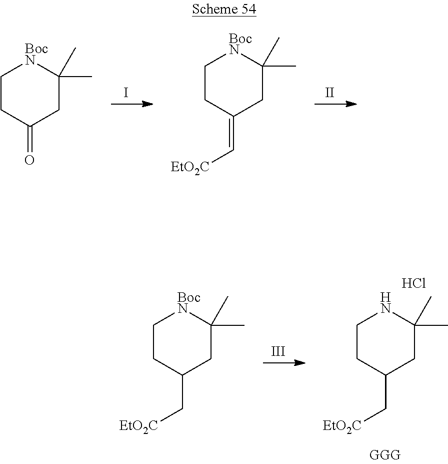

### Step I: tert-butyl (4E)-4-(2-ethoxy-2-oxo-ethylidene)-2,2-dimethyl-piperidine-1-carboxylate and tert-butyl (4Z)-4-(2-ethoxy-2-oxo-ethylidene)-2,2-dimethyl-piperidine-1-carboxylate

A stirred solution of triethylphosphonoacetate (3.157 g, 2.794 mL, 14.08 mmol) in THF (24 mL) was slowly treated with n-BuLi (5.280 mL of 2.5 M, 13.20 mmol) at −78° C., and the reaction mixture was stirred for 45 min. A solution of tert-butyl 2,2-dimethyl-4-oxo-piperidine-1-carboxylate (2 g, 8.799 mmol) in THF (20 mL) was then added to the mixture, and the reaction was left warming up to room temperature overnight, after which the conversion. The reaction mixture was then diluted with ammonium chloride and extracted three times with EtOAc, dried over sodium sulfate and filtered, and the filtrate was evaporated under reduced pressure. The residue was purified by silica gel chromatography on a 50 g column eluting with 0 to 10% EtOAc/Hexanes affording the title compound as a colorless oil, tert-butyl (4E)-4-(2-ethoxy-2-oxo-ethylidene)-2,2-dimethyl-piperidine-1-carboxylate (2.50 g, 95%) By NMR the product consisted of a 3:1 mixture of isomers. Major isomer: ¹H NMR (400 MHz, Chloroform-d) δ 5.70-5.62 (m, 1H), 4.09 (q, 2H), 3.58 (t, 2H), 2.89 (dt, J=6.8, 3.0 Hz, 2H), 2.44-2.33 (m, 2H), 1.42 (s, 9H), 1.35 (s, 6H), 1.23 (t, J=7.1 Hz, 3H).

### Step II: tert-butyl 4-(2-ethoxy-2-oxo-ethyl)-2,2-dimethyl-piperidine-1-carboxylate ethyl 2-(2,2-dimethyl-4-piperidyl)acetate

A solution of tert-butyl (4E)-4-(2-ethoxy-2-oxo-ethylidene)-2,2-dimethyl-piperidine-1-carboxylate (500 mg, 1.681 mmol) in EtOH (5 mL) was added palladium on carbon (357.8 mg, 0.3362 mmol), the reaction vessel was equipped with an H2-balloon (1 atm) and the reaction mixture was stirred overnight. Conversion was only partial after 24 h, therefore extra Pd/C was added (0.20 equiv.) and the reaction mixture was stirred for an extra 24 h. The solid was then filtered through a celite pad and the solvent was removed in vacuo to afford tert-butyl 4-(2-ethoxy-2-oxo-ethyl)-2,2-dimethyl-piperidine-1-carboxylate (503 mg, 100%) 1H NMR (400 MHz, Chloroform-d) δ 4.05 (qd, J=7.1, 1.8 Hz, 2H), 3.68 (ddd, J=13.7, 5.5, 4.5 Hz, 1H), 3.02 (ddd, J=13.9, 10.3, 3.8 Hz, 1H), 2.17-2.10 (m, 2H), 2.06-1.97 (m, 1H), 1.76-1.65 (m, 1H), 1.41 (s, 3H), 1.38 (s, 1H), 1.36 (s, 9H), 1.27-1.23 (m, 1H), 1.20 (s, 3H), 1.17 (t, J=7.1 Hz, 3H), 1.10-1.03 (m, 1H). ESI-MS m/z calc. 299.20966, found 300.47 (M+1)+.

### Step III: tert-butyl 4-(2-ethoxy-2-oxo-ethyl)-2,2-dimethyl-piperidine-1-carboxylate ethyl 2-(2,2-dimethyl-4-piperidyl)acetate

The mixture from the previous step was taken up in dioxane (5 mL) and HCl in dioxane was added (2.1 mL of 4 M, 8.4 mmol) at room temperature, then stirred for 16 h. The solvent was removed under reduced pressure to afford ethyl 2-(2,2-dimethyl-4-piperidyl)acetate hydrochloride salt (455 mg, 99%) 1H NMR (400 MHz. Chloroform-d) δ 9.26 (s, 1H), 9.04 (s, 1H), 4.00-3.81 (m, 2H), 3.16-2.99 (m, 1H), 2.80 (t, J=11.9 Hz, 1H), 2.16-1.90 (m, 3H), 1.72-1.59 (m, 1H), 1.55-1.41 (m, 3H), 1.33 (s, 3H), 1.20 (s, 3H), 1.00 (t, J=7.1 Hz, 3H). ESI-MS m/z calc. 199.15723, found 200.06 (M+1)+.

### Preparation of Intermediate III: 1-[1-[(4-methoxyphenyl)methyl]pyrazol-4-yl]-3,3-dimethyl-piperazin-2-one (Hydrochloride Salt)

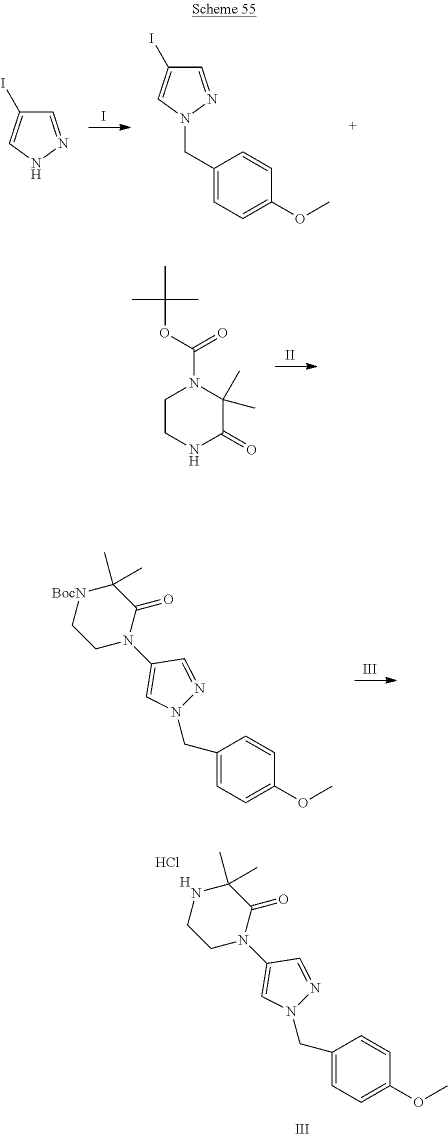

### Step I: 4-iodo-1-[(4-methoxyphenyl)methyl]pyrazole

A mixture of 4-iodo-1H-pyrazole (26 g, 134.0 mmol). 1-(chloromethyl)-4-methoxy-benzene (30.1 g, 192.4 mmol), cesium carbonate (64.7 g, 198.6 mmol) in DMF (267 mL) was heated at 60° C. overnight. The reaction mixture was diluted with 250 mL water and extracted with 2×250 mL ethyl acetate. The combined organic layers were washed with brine then dried over Na₂SO₄. The mixture was filtered then evaporated and the residue pre-adsorbed on silica gel. Purified by silica gel plug filtration using 0-10% EtOAc/Hexanes as eluent.

### Step II: 1-[1-[(4-methoxyphenyl)methyl]pyrazol-4-yl]-3,3-dimethyl-piperazin-2-one

A mixture of tert-butyl 2,2-dimethyl-3-oxo-piperazine-1-carboxylate (3.42 g, 15 mmol), 4-iodo-1-[(4-methoxyphenyl)methyl]pyrazole (5.18 g, 16.5 mmol), iodocopper (1.43 g, 7.50 mmol), N,N′-dimethylethane-1,2-diamine (1.322 g, 1.597 mL, 15.00 mmol), K₃PO₄ (6.368 g, 30.00 mmol) in DMF (78.75 mL) was heated at 120 C for 4 h. The reaction mixture was diluted with water (150 mL) and extracted (2×) with EtOAc (2×150 mL). The combined organics were washed with water (150 mL) and brine (100 mL), evaporated in vacuo and purified by flash column chromatography.

To the material obtained above, in dioxane (34.2 mL) and MeOH (1.7 mL) was added HCl (18.8 mL, 4 M, 75.0 mmol). The reaction was allowed to stir at RT for 4 h. The solvent was removed in vacuo to give a gummy solid. This material was triturated with DCM (200 mL) to give a white suspension which is suction-filtered to give a white powder. 1-[1-[(4-methoxyphenyl)methyl]pyrazol-4-yl]-3,3-dimethyl-piperazin-2-one (Hydrochloride salt) (5.41 g, 103%) ¹H NMR (400 MHz, DMSO-d6) δ 10.18 (s, 2H), 8.13 (d, J=0.8 Hz, 1H), 7.66 (d, J=0.8 Hz, 1H), 7.24-7.11 (m, 2H), 6.93-6.75 (m, 2H), 5.19 (s, 2H), 3.93-3.83 (m, 2H), 3.69 (s, 3H), 3.58-3.45 (m, 2H), 1.55 (s, 6H).

### Intermediate JJJ: Methyl 5-iodo-1-[(4-methoxyphenyl)methyl]pyrazole-3-carboxylate

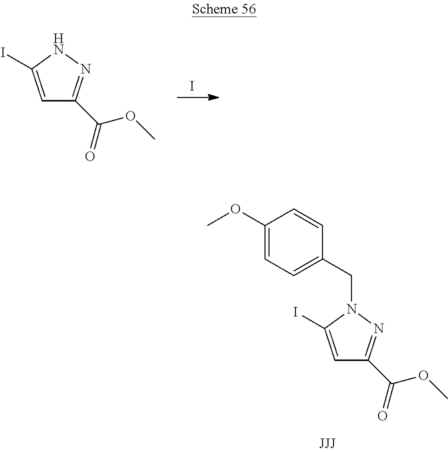

1-(Chloromethyl)-4-methoxy-benzene (985.2 mg, 853 μL, 6.291 mmol) was added to a stirred mixture of methyl 5-iodo-1H-pyrazole-3-carboxylate (1057 mg, 4.14 mmol) and Cs₂CO₃ (2.73 g, 8.39 mmol) in DMF (14 mL). The reaction mixture was stirred at 60° C. for 2 h. LC-MS analysis showed reaction completion. The reaction mixture was cooled to rt and quenched with water. Aqueous phase was extracted twice with EtOAc. The combined organic layers were washed with water/brine solution, dried over Na₂SO₄ and evaporated in vacuo. The recovered crude compound was purified on Biotage SNAP 25 g silica gel cartridge eluting with 0-20% EtOAc/Hexanes (loading with benzene) to give methyl 5-iodo-1-[(4-methoxyphenyl)methyl]pyrazole-3-carboxylate (1.15 g). ¹H NMR (400 MHz, DMSO-d6) δ 7.15-7.09 (m, 2H), 7.09-7.05 (m, 1H), 6.94-6.79 (m, 2H), 5.58 (s, 2H), 3.79 (s, 3H), 3.69 (s, 3H).

### Intermediate LLL: 3,3-dimethyl-1-(2-pyridyl)piperazine (HCl Salt)

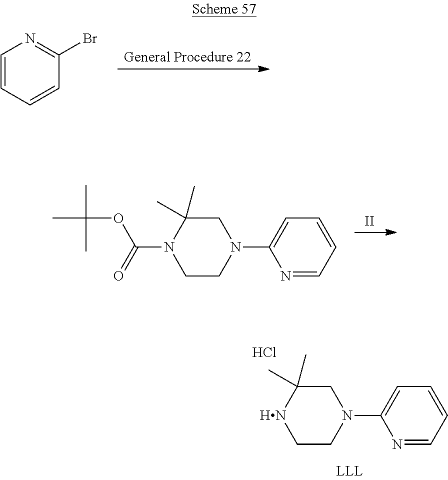

### Step I: tert-butyl 2,2-dimethyl-4-(2-pyridyl)piperazine-1-carboxylate

The title compound was prepared based on General Procedure 22, using tert-butyl 2,2-dimethylpiperazine-1-carboxylate (Hydrochloric Acid (1)) (238 mg, 0.949 mmol), rac-BINAP (24 mg, 0.03854 mmol), Pd₂(dba)₃ (17 mg, 0.01881 mmol) and NaOtBu (275 mg, 2.86 mmol) in toluene at 70° C. in a microwave vial. The crude was purified using flash chromatography eluting with EtOAc/hexanes in a gradient of 0-30% in 15 CV to provide tert-butyl 2,2-dimethyl-4-(2-pyridyl)piperazine-1-carboxylate (258 mg, 93%) as a bright yellow waxy solid. ¹H NMR (400 MHz, cdcl3) δ 8.15 (ddd, J=4.9, 2.0, 0.8 Hz, 1H), 7.48 (ddd, J=8.8, 7.1, 2.0 Hz, 1H), 6.57 (ddd, J=7.0, 4.9, 0.6 Hz, 1H), 6.42 (d, J=8.6 Hz, 1H), 3.91-3.83 (m, 2H), 3.81 (s, 2H), 3.52-3.43 (m, 2H), 1.49 (s, 9H), 1.40 (s, 6H).

### Step II: 3,3-dimethyl-1-(2-pyridyl)piperazine

To a solution of tert-butyl 2,2-dimethyl-4-(2-pyridyl)piperazine-1-carboxylate (258 mg, 0.8854 mmol) in dioxane (1.3 mL) was added 4M HCl in dioxane (2.2 mL of 4 M, 8.80 mmol). After 3.5 h, the mixture was concentrated to dryness to obtain 3,3-dimethyl-1-(2-pyridyl)piperazine (Hydrochloride salt) (231 mg, 115%) was obtained as an off-white solid. ¹H NMR (400 MHz, dmso) δ 9.25 (broad s, 2H), 8.18-8.02 (m, 1H), 7.71 (broad s, 1H), 7.07 (broad s, 1H), 6.89-6.63 (m, 1H), 3.77 (broad m, 2H), 3.65 (broad s, 2H), 3.23 (broad s, 2H), 1.33 (s, 6H).

### Preparation of Intermediate MMM: tert-butyl 6-(3,3-dimethylpiperazin-1-yl)pyridine-2-carboxylate

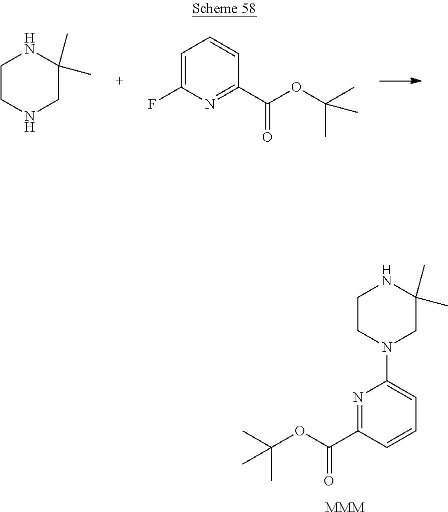

The intermediate was prepared according to General Procedure 22A using a solution of tert-butyl 6-fluoropyridine-2-carboxylate (30.05 g, 152.4 mmol), 2,2-dimethylpiperazine (21.77 g, 190.6 mmol) and cesium carbonate (61.02 g, 187.3 mmol) in NMP (300 mL) at 100° C. overnight.

### Preparation of Intermediate NNN: tert-butyl 6-(3,3-dimethylpiperazin-1-yl)pyridine-2-carboxylate

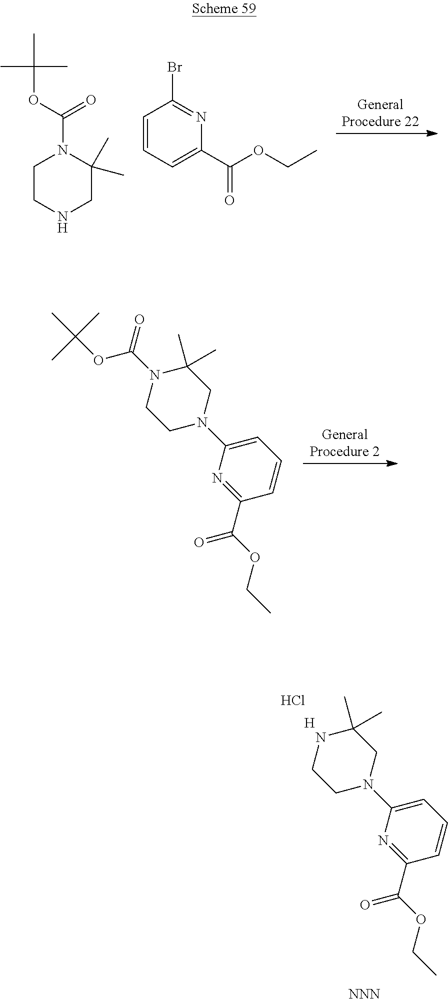

A mixture of tert-butyl 2,2-dimethylpiperazine-1-carboxylate (837.8 mg, 3.909 mmol), ethyl 6-bromopyridine-2-carboxylate (989.3 mg, 4.30 mmol). Cs₂CO₃ (3.184 g, 9.77 mmol), in NMP (8.38 mL) was heated o/n 120 C. Reaction went dry. The reaction mixture was diluted with DCM (2 mL) and purified on silica column eluting with 10-40% ethylacetate:hexanes to afford the N-Boc intermediate (254 mg, 18%). To tert-butyl 4-(6-ethoxycarbonyl-2-pyridyl)-2,2-dimethyl-piperazine-1-carboxylate in dioxane (8.4 mL) and MeOH (837.8 μL) was then added HCl (977.2 μL of 4 M, 3.909 mmol) and the reaction mixture stirred at rt overnight. The solvent was evaporated in vacuo to give the crude title product.

### Preparation of Intermediate OOO: (7-tert-butyl-5-chloro-furo[3,2-b]pyridin-2-yl)methanol

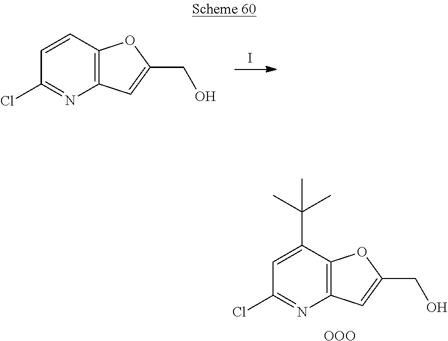

### Step I: (7-tert-butyl-5-chloro-furo[3,2-b]pyridin-2-yl)methanol

To a mixture of (5-chlorofuro[3,2-b]pyridin-2-yl)methanol (2.78 g, 15.14 mmol), 2,2-dimethylpropanoic acid (6.185 g, 3.5 mL, 60.6 mmol), water (13.90 mL), TFA (2.589 g, 1.75 mL, 22.71 mmol) and acetonitrile (28 mL) was added siver nitrate (1.286 g, 7.57 mmol). The reaction mixture was heated at 80° C. in the dark. A solution of ammonium persulfate (5.754 g, 27.25 mmol) in water (14 mL) was added dropwise to the reaction mixture and it is stirred at 80° C. for 30 minutes. Another solution of ammonium persulfate (5.754 g, 27.25 mmol) in water (14 mL) was added dropwise to the reaction mixture and it is stirred at 80° C. for an extra 30 minutes. The reaction mixture was cooled to room temperature and diluted with isopropyl acetate. The resulting bi-phasic mixture was allowed to cool down to room temperature, whereupon it was filtered over celite. The filtrate was cooled to 0° C. and basified to pH 9 by charging NH₄OH dropwise under strong stirring. The solution was stirred for 30 minutes and the layers were separated. The organic layer was washed with aqueous NaOH 1N and brine. The organic phase was concentrated under reduced pressure and the residue was purified by flash chromatography on silica gel eluting with 10-40% EtOAc: Hexanes affording the title compound: (7-tert-butyl-5-chloro-furo[3,2-b]pyridin-2-yl)methanol (833 mg, 23% yield). ¹H NMR (400 MHz, DMSO-d6) δ 7.15 (s, 1H), 6.90-6.85 (m, 1H), 5.66 (t, J=6.0 Hz, 1H), 4.64 (dd, J=6.0, 0.9 Hz, 2H), 1.45 (s, 9H) ESI-MS m/z calc. 239.07, found 239.91 (M+1)⁺; Retention time: 1.43 minutes using method C.

### Preparation of intermediate PPP: (7-(tert-butyl)-5-(4,4-difluorocyclohexyl)furo[3,2-b]pyridin-2-yl)(2,2-dimethylpiperazin-1-yl)methanone

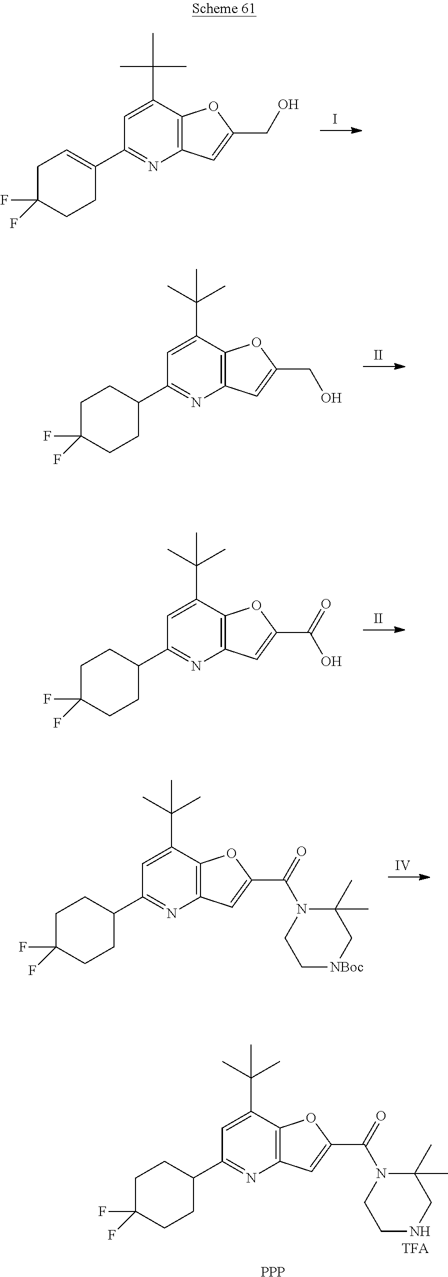

### Step I: [7-tert-butyl-5-(4,4-difluorocyclohexyl)furo[3,2-b]pyridin-2-yl]methanol

To a flask containing [7-tert-butyl-5-(4,4-difluorocyclohexen-1-yl)furo[3,2-b]pyridin-2-yl]methanol (788 mg, 2.45 mmol) and Pd—C (261 mg, 0.245 mmol) in EtOH (24.52 mL) was hydrogenated (balloon) at room temperature overnight. The suspension was filtered over celite, washed with DCM and the volatiles were removed under reduced pressure to afford [7-tert-butyl-5-(4,4-difluorocyclohexyl)furo[3,2-b]pyridin-2-yl]methanol (793 mg, 100%).

### Step II. 7-(tert-butyl)-5-(4,4-difluorocyclohexyl)furo[3,2-b]pyridine-2-carboxylic acid

A mixture of [7-tert-butyl-5-(4,4-difluorocyclohexyl)furo[3,2-b]pyridin-2-yl]methanol (793 mg, 2.45 mmol) and 4-methyl-4-oxido-morpholin-4-ium hydrate (Water (1)) (3.756 g, 24.52 mmol) in MeCN (11.1 mL) was treated with TPAP (86.2 mg, 0.245 mmol). The reaction mixture was treated with iPrOH (1.49 g, 1.89 mL, 24.7 mmol) after ca. 1 h the reaction mixture was diluted with H₂O and EtOAc and the phases were separated. The solvent was removed in vacuo to afford 7-tert-butyl-5-(4,4-difluorocyclohexyl)furo[3,2-b]pyridine-2-carboxylic acid (827 mg, 100%). ESI-MS m/z calc. 337.14896, found: 338.08. Rt: 1.46 min using Method C.

### Step III: tert-butyl 4-[7-tert-butyl-5-(4,4-difluorocyclohexyl)furo[3,2-b]pyridine-2-carbonyl]-3,3-dimethyl-piperazine-1-carboxylate

7-tert-butyl-5-(4,4-difluorocyclohexyl)furo[3,2-b]pyridine-2-carboxylic acid (910 mg, 2.697 mmol) and tert-butyl 3,3-dimethylpiperazine-1-carboxylate (578.0 mg, 2.697 mmol) was dissolved in DMF (3.3 mL). HATU (1.33 g, 3.51 mmol) and DIPEA (1.64 mL, 9.44 mmol) were successively added at RT. The reaction mixture was stirred overnight. EtOAc along with water were added. The phases were separated, and concentrated in vacuo to afford the title compound (950 mg, 66%). ESI-MS m/z calc. 533.3065, found 534.21. Rt: 2.48 min using Method C.

### Step IV: [7-tert-butyl-5-(4,4-difluorocyclohexyl)furo[3,2-b]pyridin-2-yl]-(2,2-dimethylpiperazin-1-yl)methanone

tert-butyl 4-[7-tert-butyl-5-(4,4-difluorocyclohexyl)furo[3,2-b]pyridine-2-carbonyl]-3,3-dimethyl-piperazine-1-carboxylate (500 mg, 0.937 mmol) was dissolved in DCM (3.1 mL) and TFA (1.44 mL, 18.7 mmol) was added at RT. The reaction mixture was stirred for 30 min. The solvents were removed under reduced pressure to afford [7-tert-butyl-5-(4,4-difluorocyclohexyl)furo[3,2-b]pyridin-2-yl]-(2,2-dimethylpiperazin-1-yl)methanone TFA salt (406.2 mg, 100%). ESI-MS m/z calc. 433.2541, found: 434.34.

### Preparation of Intermediates QQQ and RRR

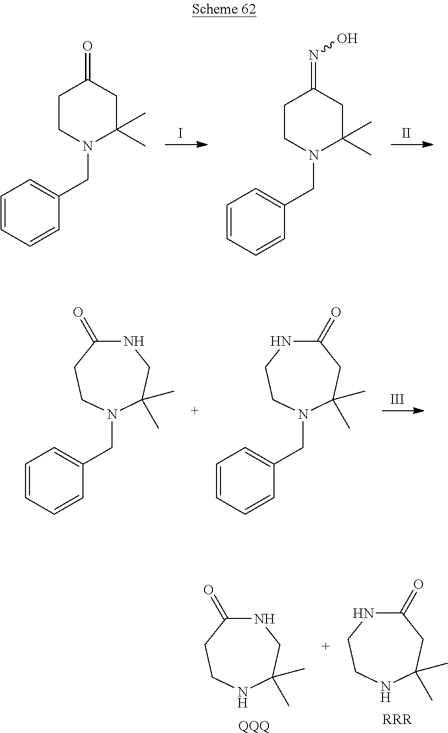

### Step I: 1-benzyl-2,2-dimethyl-piperidin-4-one oxime

1-benzyl-2,2-dimethyl-piperidin-4-one (Hydrochloric Acid (1)) (200 mg, 0.7881 mmol) was dissolved in EtOH (4 mL) in a 5 mL microwave flask. Hydroxylamine hydrochloride (110 mg, 1.58 mmol) followed by Et₃N (330 μL, 2.37 mmol) were added and the flask was capped. The reaction mixture was submitted to microwave irradiation at 120° C. for 30 minutes. Water was added along with EtOAc and the phases were separated. The organic phase was washed two other times with water and then sat. brine, dried over MgSO4, filtered and evaporated under reduced pressure. The residue was left under vacuum overnight to afford 1-benzyl-2,2-dimethyl-piperidin-4-one oxime (171 mg, 93%) 1H NMR (400 MHz, DMSO-d6) δ 10.23 (d, J=24.6 Hz, 1H), 7.38-7.25 (m, 4H), 7.26-7.16 (m, 1H), 3.51 (s, 2H), 2.48-2.42 (m, 1H), 2.41-2.29 (m, 3H), 2.11 (s, 1H), 2.07 (dd, J=6.6, 5.4 Hz, 1H), 1.12-1.02 (m, 6H).

### Step II: 1-benzyl-7,7-dimethyl-1,4-diazepan-5-one and 1-benzyl-2,2-dimethyl-1,4-diazepan-5-one

To a solution of 1-benzyl-2,2-dimethyl-piperidin-4-one oxime (171 mg, 0.736 mmol) and Na₂CO₃ (312.0 mg, 2.94 mmol) in a mixture of acetone (4 mL) and water (4 mL) was added benzenesulfonyl chloride (188 μL, 1.47 mmol) dropwise at 0° C. The reaction was allowed to warm to r.t. and was then stirred 18 h. Water was added along with EtOAc and the phases were separated. The organic phase was washed water (×2) and brine, dried over MgSO4, filtered and evaporated under reduced pressure to afford a 1:4 mixture regioisomers 1-benzyl-7,7-dimethyl-1,4-diazepan-5-one and 1-benzyl-2,2-dimethyl-1,4-diazepan-5-one. Major Regioisomer (1-benzyl-2,2-dimethyl-1,4-diazepan-5-one): ¹H NMR (400 MHz. Chloroform-d) δ 7.43-7.18 (m, 5H), 6.26 (s, 1H), 3.58 (s, 2H), 3.29-3.12 (m, 2H), 2.76-2.57 (m, 4H), 1.16 (s, 6H).

Minor Regioisomer (1-benzyl-7,7-dimethyl-1,4-diazepan-5-one): 1H NMR (400 MHz, Chloroform-d) δ 7.43-7.18 (m, 5H), 6.34 (s, 1H), 3.63 (s, 2H), 3.29-3.12 (m, 2H), 2.76-2.38 (m, 4H), 1.30-1.26 (m, 6H).

### Step III: 2,2-dimethyl-1,4-diazepan-5-one and 7,7-dimethyl-1,4-diazepan-5-one

To a solution of 1-benzyl-2,2-dimethyl-1,4-diazepan-5-one (1.01 g, 4.347 mmol) in Ethanol (21.74 mL) were added Methanesulfonic Acid (438.6 mg, 296.2 μL, 4.564 mmol) and Pd(OH)₂ (122.1 mg, 0.8694 mmol). The reaction flask was flushed with N₂ twice (vacuum and N₂) and the mixture was hydrogenated using a H₂ balloon and stirred at rt for 18 h (ON). The mixture was filtered and the solvent was removed under reduced pressure to afford 1.19 g (115%) of a mixture of 7,7-dimethyl-1,4-diazepan-5-one and 2,2-dimethyl-1,4-diazepan-5-one (1:4) as an off-white solid. ESI-MS m/z calc. 142.11061, found 143.22 (M+1)+;

### Intermediate SSS: 1-[2-[(4-methoxyphenyl)methyl]-5-methyl-pyrazol-3-yl]-3,3-dimethyl-piperazin-2-one (Hydrochloric Acid (1))

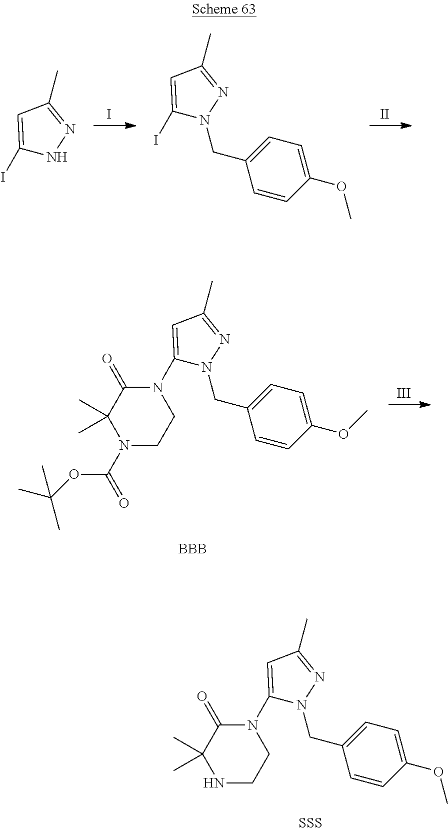

### Step I: 5-Iodo-1-[(4-methoxyphenyl)methyl]-3-methyl-pyrazole

1-(chloromethyl)-4-methoxy-benzene (2.25 g, 1.96 mL, 14.42 mmol) was added to a stirred mixture of 3-iodo-5-methyl-1H-pyrazole (2 g, 9.62 mmol) and Cs₂CO₃ (6.27 g, 19.24 mmol) in DMF (30 mL). After stirring at 60° C. for about 2 h, it was cooled down to room temperature, diluted with H₂O (100 mL) and extracted with EtOAc (3×50 mL). The combined organic extracts were washed with saturated NH₄Cl (25 mL), brine (25 mL), dried over MgSO₄, filtered and concentrated. The residue was purified on silica gel chromatography (100 g), eluting with EtOAc in hexanes. 0% (1CV), 0-20% (1° CV), 20% (2CV), to provide 5-iodo-1-[(4-methoxyphenyl)methyl]-3-methyl-pyrazole (2.97 g, 94%) as a white solid as a 3.7:1 mixture of regioisomers. For major regioisomer: ¹H NMR (400 MHz, CDCl₃) δ 7.12-7.02 (m, 2H), 6.88-6.79 (m, 2H), 6.19 (s, 1H), 5.21 (s, 2H), 3.78 (s, 3H), 2.17 (s, 3H). ESI-MS m/z calc. 328.00726, found 328.93 (M+1)⁺.

### Step II: tert-Butyl 4-[2-[(4-methoxyphenyl)methyl]-5-methyl-pyrazol-3-yl]-2,2-dimethyl-3-oxo-piperazine-1-carboxylate

A mixture of tert-butyl 2,2-dimethyl-3-oxo-piperazine-1-carboxylate (625 mg, 2.74 mmol), 5-iodo-1-[(4-methoxyphenyl)methyl]-3-methyl-pyrazole (1.07 g, 3.29 mmol), iodocopper (260.7 mg, 1.369 mmol), N,N′-dimethylethane-1,2-diamine (241.4 mg, 291 μL, 2.74 mmol), potassium phosphate (1.16 g, 5.48 mmol) in DMF (14 mL) was heated at 120° C. After 3.5 h, reaction was completed. The reaction mixture was cooled and filtered to remove Cu salts. The filtrate was diluted with water and extracted 2× with ethyl acetate. The combined organic layers were dried over Na₂SO₄ and evaporated in vacuo. The recovered crude compound was then purified on silica gel chromatography (25 g) eluting with 0-15% EtOAc/Hexanes (loading with benzene) to give tert-butyl 4-[2-[(4-methoxyphenyl)methyl]-5-methyl-pyrazol-3-yl]-2,2-dimethyl-3-oxo-piperazine-1-carboxylate (1.365 g, 116%).

### Step III: 1-(1-(4-Methoxybenzyl)-3-methyl-H-pyrazol-5-yl)-3,3-dimethylpiperazin-2-one (HCl Salt)

To a stirred solution of tert-butyl 4-[2-[(4-methoxyphenyl)methyl]-5-methyl-pyrazol-3-yl]-2,2-dimethyl-3-oxo-piperazine-1-carboxylate (107 mg, 0.25 mmol) in DCM (3.0 mL)/MeOH (0.25 mL) was added 4M HCl in dioxane (312 μL of 4 M, 1.25 mmol). The reaction mixture was stirred 2 hours at room temperature and then the volatiles were removed by evaporation. The crude (1-(1-(4-methoxybenzyl)-3-methyl-1H-pyrazol-5-yl)-3,3-dimethylpiperazin-2-one (HCl salt)) was used as such for the next step.

### Preparation of Intermediate TTT: methyl 6-(3,3-dimethylpiperazin-1-yl)-4-methoxy-pyridine-3-carboxylate

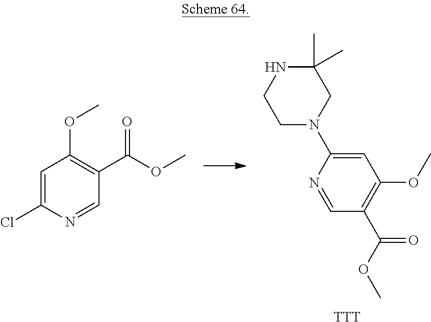 [SMILES: COC(=O)c1cnc(Cl)cc1OC ; COC(=O)c1cnc(N2CCNC(C)(C)C2)cc1OC ; "TTT"]

To a solution of tert-butyl 2,2-dimethylpiperazine-1-carboxylate (200 mg, 0.933 mmol) in dioxane (3 mL) are added methyl 6-chloro-4-methoxy-pyridine-3-carboxylate (244.5 mg, 1.21 mmol) and DIPEA (405 μL, 2.32 mmol). The mixture was heated in a microwave reactor at 160° C. for 4 h. The volatiles were removed under reduced pressure and the residue was purified silica gel chromatography (EtOAc/hexanes 0-35%6) to obtain tert-butyl 4-(4-methoxy-5-methoxycarbonyl-2-pyridyl)-2,2-dimethyl-piperazine-1-carboxylate (174 mg, 49%) as a white solid. ESI-LC-MS: calc. 380.21854; found: 380.36 Retention Time: 1.36 min using Method C.

To a solution of tert-butyl 4-(4-methoxy-5-methoxycarbonyl-2-pyridyl)-2,2-dimethyl-piperazine-1-carboxylate (174 mg, 0.459 mmol) in DCM (1 mL) was added 4M HCl/dioxane (1 mL of 4 M, 4.00 mmol). The mixture was allowed to stir at RT for 2 h. The volatiles were removed under reduced pressure to provide methyl 6-(3,3-dimethylpiperazin-1-yl)-4-methoxy-pyridine-3-carboxylate (Dihydrochloride salt) (168 mg, 104%) as a white solid. ESI-LC-MS: calc. 280.1661; found: 281.39 (M+H)⁺; RT=0.53 min using Method C.

### Preparation of Intermediate UUU: methyl 2-amino-6-(3,3-dimethylpiperazin-1-yl)pyridine-3-carboxylate

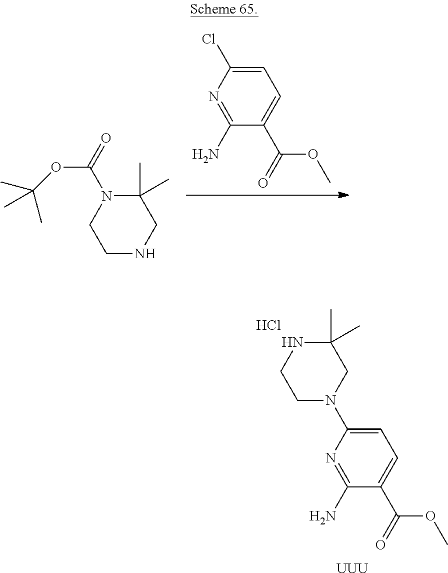 [SMILES: CC(C)(C)OC(=O)N1CCNCC1(C)C ; COC(=O)c1ccc(Cl)nc1N ; Cl ; COC(=O)c1ccc(N2CCNC(C)(C)C2)nc1N ; "UUU"]

To a solution of tert-butyl 2,2-dimethylpiperazine-1-carboxylate (280 mg, 1.31 mmol) in NMP (2 mL) were added methyl 2-amino-6-chloro-pyridine-3-carboxylate (295 mg, 1.58 mmol) and DIPEA (460 μL, 2.64 mmol). The mixture was heated in a microwave at 160° C. for 3 h. After cooling to RT, it was diluted with EtOAc (60 mL), washed with water and brine, dried over sodium sulfate and filtered. The filtrate was concentrated to dryness and the residue was purified by silica gel column chromatography (EtOAc/hexanes 0-25%) to obtain tert-butyl 4-(6-amino-5-methoxycarbonyl-2-pyridyl)-2,2-dimethyl-piperazine-1-carboxylate (270 mg, 57%) as an oil ESI-MS m/z calc. 364.21106, found 365.38 (M+1)+; Retention time: 1.65 minutes using Method C.

To a solution of tert-butyl 4-(6-amino-5-methoxycarbonyl-2-pyridyl)-2,2-dimethyl-piperazine-1-carboxylate (270 mg, 0.7409 mmol) in DCM (1 mL) was added 4M HCl/dioxane (1 mL of 4 M, 4.000 mmol). The mixture was stirred at rt overnight. The volatiles were removed under reduced pressure to afford methyl 2-amino-6-(3,3-dimethylpiperazin-1-yl)pyridine-3-carboxylate (Dihydrochloride salt) (215 mg, 86%) as a white solid. ESI-MS m/z calc. 264.15863, found: 265.18; Retention time: 0.68 minutes using Method C.

### Preparation of Intermediate VVV: ethyl 6-(3,3-dimethylpiperazin-1-yl)-4-methyl-pyridine-2-carboxylate

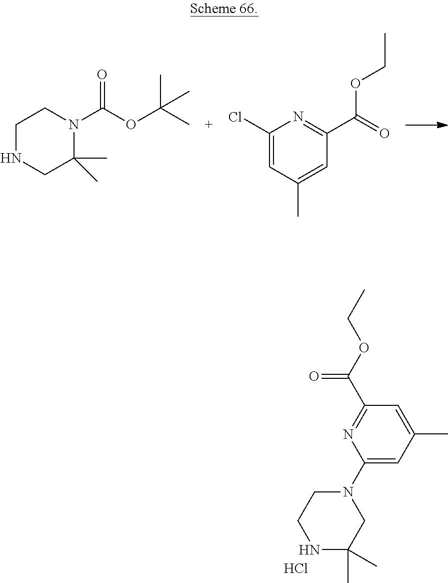 [SMILES: CC(C)(C)OC(=O)N1CCNCC1(C)C ; CCOC(=O)c1cc(C)cc(Cl)n1 ; Cl ; CCOC(=O)c1cc(C)cc(N2CCNC(C)(C)C2)n1]

According to General Procedure 22B a solution of tert-butyl 2,2-dimethylpiperazine-1-carboxylate (200 mg, 0.933 mmol) in dioxane (4 mL) were added ethyl 6-chloro-4-methyl-pyridine-2-carboxylate (242 mg, 1.21 mmol), Pd₂(dba)₃ (128 mg, 0.140 mmol), dicyclohexyl-[2-(2,4,6-triisopropylphenyl)phenyl]phosphane (89 mg, 0.19 mmol) and K₂CO₃ (258 mg, 1.87 mmol). The mixture was heated at 90° C. under nitrogen for 10 h. Then the mixture was diluted with EtOAc, filtered over a pad of celite, washed with EtOAc. The filtrate was concentrated to dryness. The residue was purified on Biotage SNAP 25 g silica gel cartridge eluting with EtOAc/hexanes in 20 CV to obtain tert-butyl 4-(6-ethoxycarbonyl-4-methyl-2-pyridyl)-2,2-dimethyl-piperazine-1-carboxylate (102 mg, 29%) as a white solid. ¹H NMR (400 MHz. Chloroform-d) δ 7.22-7.17 (m, 1H), 6.39 (t, J=1.0 Hz, 1H), 4.36 (q, J=7.1 Hz, 2H), 3.89-3.75 (m, 4H), 3.52 (dd, J=6.3, 5.1 Hz, 2H), 2.30 (s, 3H), 1.47 (s, 9H), 1.42-1.25 (m, 9H).

To a solution of tert-butyl 4-(6-ethoxycarbonyl-4-methyl-2-pyridyl)-2,2-dimethyl-piperazine-1-carboxylate (102 mg, 0.2702 mmol) in DCM (1 mL) was added 4M HCl/dioxane (500 μL of 4 M, 2.000 mmol). The mixture was stirred at RT overnight. Then the volatiles were removed under reduced pressure to afford ethyl 6-(3,3-dimethylpiperazin-1-yl)-4-methyl-pyridine-2-carboxylate (Dihydrochloride salt) (92 mg, 97%) as an off-white solid.

### Preparation of Intermediate WWW: methyl 6-(3,3-dimethylpiperazin-1-yl)-2-methoxy-pyridine-3-carboxylate

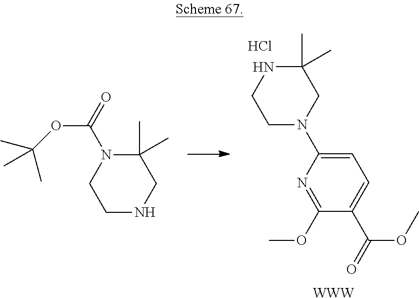

To a solution of tert-butyl 2,2-dimethylpiperazine-1-carboxylate (200 mg, 0.933 mmol) in NMP (3 mL) were added methyl 6-chloro-2-methoxy-pyridine-3-carboxylate (245 mg, 1.22 mmol) and DIPEA (490 μL, 2.813 mmol). The mixture was heated in a microwave reactor at 160° C. for 1 h. After cooling to RT, it was diluted with EtOAc (60 mL), washed with water and brine consecutively, dried over sodium sulfate, filtered and concentrated to dryness. The residue was purified on Biotage SNAP 25 g silica gel cartridge eluting with EtOAc/hexanes 0-35% in 20 CV to obtain tert-butyl 4-(6-methoxy-5-methoxycarbonyl-2-pyridyl)-2,2-dimethyl-piperazine-1-carboxylate (182 mg, 51%) as an oil.

To a solution of tert-butyl 4-(6-methoxy-5-methoxycarbonyl-2-pyridyl)-2,2-dimethyl-piperazine-1-carboxylate (182 mg, 0.4796 mmol) in DCM (1 mL) was added 4M HCl/dioxane (1 mL of 4 M. 4.000 mmol). The mixture was stirred at rt for 2 h. The volatiles were removed under reduced pressure to obtain methyl 6-(3,3-dimethylpiperazin-1-yl)-2-methoxy-pyridine-3-carboxylate (Dihydrochloride salt) (160 mg, 95%) as an off-white solid. ESI-MS m/z calc. 279.1583, found: 280.36 Retention time: 0.58 minutes using Method C.

### Example 1: 1-[4-[5-(4-Fluorophenyl)-7-isopropyl-furo[3,2-b]pyridine-2-carbonyl]-3,3-dimethyl-piperazin-1-yl]-2-hydroxy-2-methyl-propan-1-one

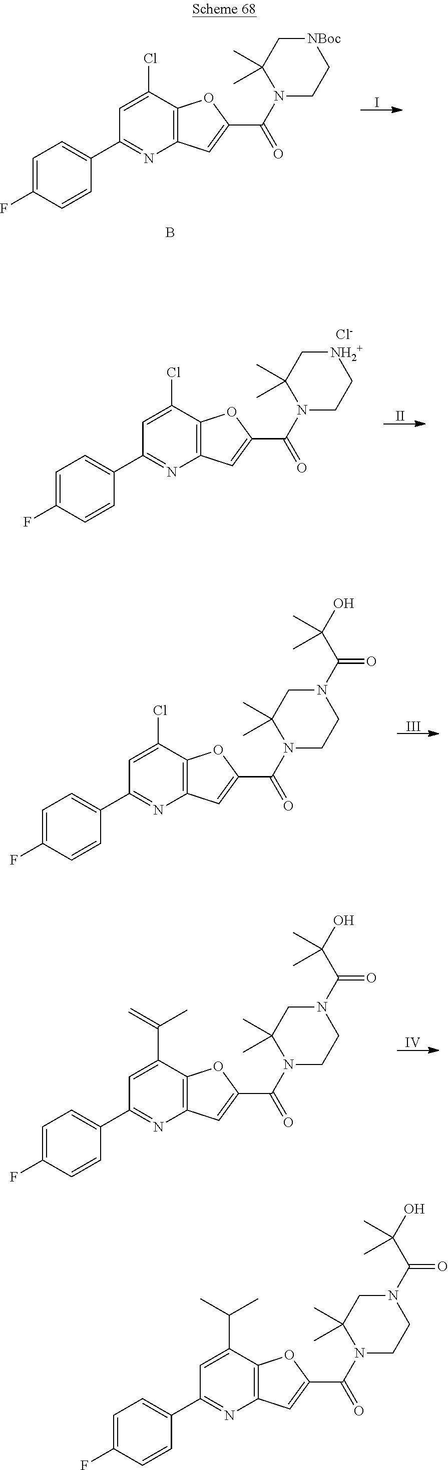

### Step I: [7-Chloro-5-(4-fluorophenyl)furo[3,2-b]pyridin-2-yl]-(2,2-dimethylpiperazin-1-yl)methanone hydrochloride

The product was prepared according to General Procedure 2 using Intermediate B (118 mg, 0.24 mmol), DCM (1.8 mL) and HCl in dioxane (907 μL of 4 M, 3.63 mmol) affording the title compound. [7-Chloro-5-(4-fluorophenyl)furo[3,2-b]pyridin-2-yl]-(2,2-dimethylpiperazin-1-yl)methanone hydrochloride (103 mg, 100% yield). ESI-MS m/z calc. 387.115, found 388.39 (M+1)⁺; Retention time: 1.31 minutes using method C.

### Step II: 1-[4-[7-Chloro-5-(4-fluorophenyl)furo[3,2-b]pyridine-2-carbonyl]-3,3-dimethyl-piperazin-1-yl]-2-hydroxy-2-methyl-propan-1-one

The product was prepared according to General Procedure 3 using [7-chloro-5-(4-fluorophenyl)furo[3,2-b]pyridin-2-yl]-(2,2-dimethylpiperazin-1-yl)methanone hydrochloride (103 mg, 0.24 mmol), HATU (111 mg, 0.29 mmol), DMF (6.2 mL), Hünig's base (157 mg, 212 μL, 1.21 mmol) and 2-hydroxy-2-methyl-propanoic acid (28 mg, 0.27 mmol) affording the title compound which was used in the subsequent step without purification. 1-[4-[7-Chloro-5-(4-fluorophenyl)furo[3,2-b]pyridine-2-carbonyl]-3,3-dimethyl-piperazin-1-yl]-2-hydroxy-2-methyl-propan-1-one (44 mg, 38% yield). ESI-MS m/z calc. 473.15176, found 474.45 (M+1)⁺; Retention time: 1.65 minutes using method C.

### Step III: 1-[(4-[5-(4-Fluorophenyl)-7-isopropenyl-furo[3,2-b]pyridine-2-carbonyl]-3,3-dimethyl-piperazin-1-yl]-2-hydroxy-2-methyl-propan-1-one

To a microwave vial containing 1-[4-[7-chloro-5-(4-fluorophenyl)furo[3,2-b]pyridine-2-carbonyl]-3,3-dimethyl-piperazin-1-yl]-2-hydroxy-2-methyl-propan-1-one (40 mg, 0.084 mmol), Pd₂dba₃ (1.2 mg, 0.0013 mmol) and S-Phos (2.1 mg, 0.0051 mmol) was added PhMe (0.4 mL), 2-isopropenyl-4,4,5,5-tetramethyl-1,3,2-dioxaborolane (21 mg, 24 μL, 0.13 mmol) and K₃PO₄ (127 μL of 2 M, 0.25 mmol). The vial put under an atmosphere of N₂ before it was sealed and stirred at 120° C. for 45 min under microwave irradiation. The solution was cooled to room temperature and the pH of the solution adjusted to 8 using 1N HCl and aq. NaHCO₃. The organic phase was extracted with DCM and the combined organic phase washed with brine, dried over MgSO₄, filtered and the filtrate evaporated under reduced pressure affording the title compound which was used in the subsequent step without further purification. 1-[4-[5-(4-Fluorophenyl)-7-isopropenyl-furo[3,2-b]pyridine-2-carbonyl]-3,3-dimethyl-piperazin-1-yl]-2-hydroxy-2-methyl-propan-1-one (40 mg, 99% yield). ESI-MS m/z calc. 479.22203, found 480.72 (M+1)⁺; Retention time: 1.71 minutes using method C.

### Step IV: 1-[4-[5-(4-Fluorophenyl)-7-iso-propyl-furo[3,2-b]pyridine-2-carbonyl]-3,3-dimethyl-piperazin-1-yl]-2-hydroxy-2-methyl-propan-1-one

To a flask containing palladium hydroxide on carbon (5.86 mg, 0.0042 mmol) and 1-[4-[5-(4-fluorophenyl)-7-isopropenyl-furo[3,2-b]pyridine-2-carbonyl]-3,3-dimethyl-piperazin-1-yl]-2-hydroxy-2-methyl-propan-1-one (40 mg, 0.083 mmol) under a nitrogen atmosphere was added EtOAc (834 μL). The flask was evacuated under reduced pressure and re-filled with hydrogen 3 times and the suspension was stirred under a hydrogen pressure at room temperature for 48 h. Hydrogen was evacuated and the flask backfilled with nitrogen. The catalyst was filtered on Celite® and washed with DCM and the filtrate was evaporated under reduced pressure. The residue was purified by flash chromatography on silica gel eluting with 20-80% EtOAc: Hexanes affording the title compound. 1-[4-[5-(4-Fluorophenyl)-7-isopropyl-furo[3,2-b]pyridine-2-carbonyl]-3,3-dimethyl-piperazin-1-yl]-2-hydroxy-2-methyl-propan-1-one (17 mg, 40% yield). ¹H NMR (400 MHz, Chloroform-d) δ 7.99-7.91 (m, 2H), 7.53 (s, 1H), 7.39 (s, 1H), 7.20-7.11 (m, 2H), 4.03-3.71 (m, 6H), 3.44 (hept, J=6.7 Hz, 1H), 1.64 (s, 7H), 1.53 (s, 6H), 1.44 (d, J=7.0 Hz, 6H). ESI-MS m/z calc. 481.23767, found 482.46 (M+1)⁺; Retention time: 1.71 minutes using method C.

### Example 2: 1-[4-[5-(4-Fluorophenyl)-7-methyl-furo[3,2-b]pyridine-2-carbonyl]-3,3-dimethyl-piperazin-1-yl]-2-hydroxy-2-methyl-propan-1-one

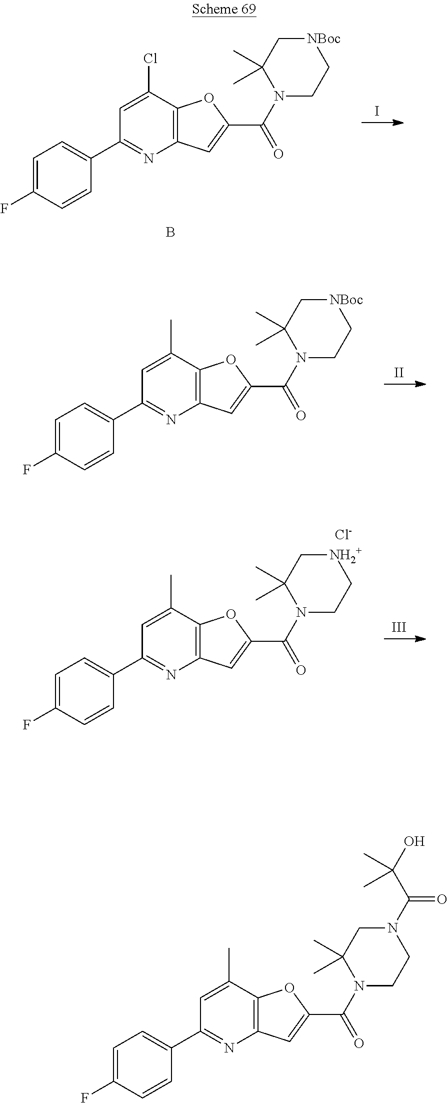

### Step I: tert-Butyl 4-[5-(4-fluorophenyl)-7-vinyl-furo[3,2-b]pyridine-2-carbonyl]-3,3-dimethyl-piperazine-1-carboxylate

To a round bottom flask containing a solution of Intermediate B (55 mg, 0.10 mmol) in dioxane (1.1 mL) was added Pd₂dba₃ (0.96 mg, 0.0010 mmol) and S-Phos (1.64 mg, 0.0040 mmol) and the solution was degassed with nitrogen for 5 minutes. K₃PO₄ (210 μL of 2 M, 0.42 mmol) and methylboronic acid (10 mg, 0.17 mmol) were added and the solution was stirred at 90° C. for 16 h. The solution was cooled to room temperature before aq. NaHCO₃ was added and the aq. phase was extracted with DCM. The combined organic phase was washed with brine, dried over MgSO₄, filtered and the filtrate evaporated under reduced pressure affording the title compound which was used in the subsequent step without further purification. tert-Butyl-4-[5-(4-fluorophenyl)-7-methyl-furo[3,2-b]pyridine-2-carbonyl]-3,3-dimethyl-piperazine-1-carboxylate (50 mg, 100% yield). ESI-MS m/z calc. 467.22203, found 468.74 (M+1)⁺; Retention time: 2.18 minutes using method C.

### Step II: (2,2-Dimethylpiperazin-1-yl)-[5-(4-fluorophenyl)-7-methyl-furo[3,2-b]pyridin-2-yl]methanone hydrochloride

The product was prepared according to General Procedure 2 using tert-butyl-4-[5-(4-fluorophenyl)-7-methyl-furo[3,2-b]pyridine-2-carbonyl]-3,3-dimethyl-piperazine-1-carboxylate (50 mg, 0.10 mmol), DCM (1.1 mL) and HCl in dioxane (535 μL of 4 M, 2.14 mmol) affording the title compound. (2,2-Dimethylpiperazin-1-yl)-[5-(4-fluorophenyl)-7-methyl-furo[3,2-b]pyridin-2-yl]methanone hydrochloride (43 mg, 100% yield). ESI-MS m/z calc. 367.16961, found 368.37 (M+1)⁺; Retention time: 1.33 minutes using method C.

### Step III: 1-[4-[5-(4-Fluorophenyl)-7-methyl-furo[3,2-b]pyridine-2-carbonyl]-3,3-dimethyl-piperazin-1-yl]-2-hydroxy-2-methyl-propan-1-one

The product was prepared according to General Procedure 3 using (2,2-dimethylpiperazin-1-yl)-[5-(4-fluorophenyl)-7-methyl-furo[3,2-b]pyridin-2-yl]methanone hydrochloride (43 mg, 0.11 mmol), HATU (57 mg, 0.15 mmol), DMF (888 μL), Hünig's base (111 μL, 0.64 mmol) and 2-hydroxy-2-methyl-propanoic acid (14 mg, 0.14 mmol) affording the title compound. 1-[4-[5-(4-Fluorophenyl)-7-methyl-furo[3,2-b]pyridine-2-carbonyl]-3,3-dimethyl-piperazin-1-yl]-2-hydroxy-2-methyl-propan-1-one (20 mg, 41% yield). ¹H NMR (400 MHz, DMSO-d6) δ 8.19-8.07 (m, 2H), 7.89 (s, 1H), 7.46 (s, 1H), 7.36-7.23 (m, 2H), 5.40 (s, 1H), 4.26-3.39 (m, 6H), 2.56 (s, 3H), 1.55-1.44 (m, 6H), 1.32 (s, 6H). ESI-MS m/z calc. 453.2064, found 454.58 (M+1)⁺; Retention time: 3.97 minutes using method D.

### Example 3: 1-[4-[7-Ethyl-5-(4-fluorophenyl)furo[3,2-b]pyridine-2-carbonyl]-3,3-dimethyl-piperazin-1-yl]-2-hydroxy-2-methyl-propan-1-one

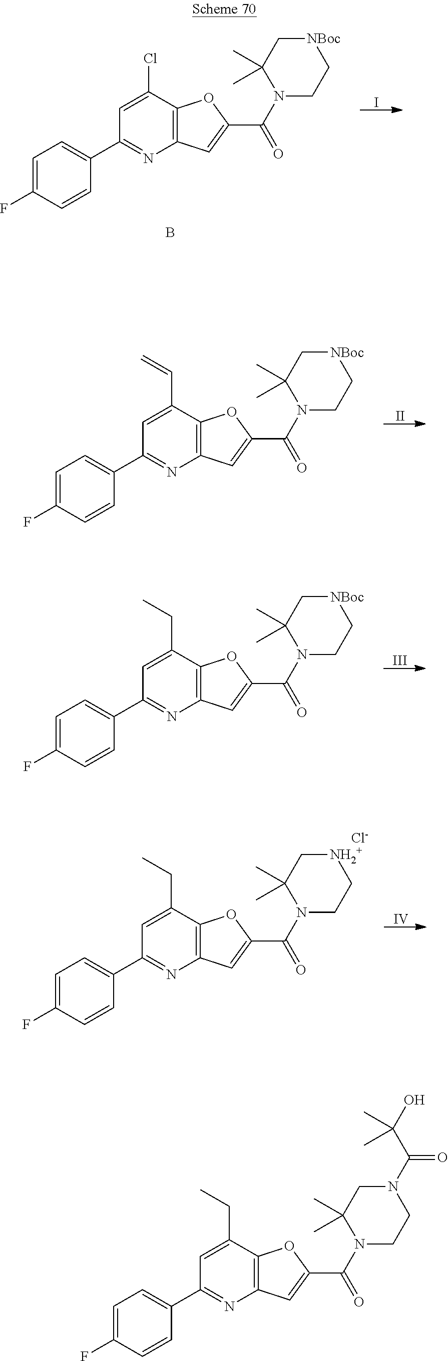

### Step I: tert-Butyl-4-[5-(4-fluorophenyl)-7-vinyl-furo[3,2-b]pyridine-2-carbonyl]-3,3-dimethyl-piperazine-1-carboxylate

To a round bottom flask containing a solution of Intermediate B (55 mg, 0.10 mmol) in dioxane (1.1 mL) was added Pd₂dba₃ (0.96 mg, 0.0010 mmol) and S-Phos (1.63 mg, 0.0040 mmol) and the solution was degassed with nitrogen for 5 minutes. K₃PO₄ (210 μL of 2 M, 0.42 mmol) and 2,4,6-Trivinylcyclotriboroxane pyridine complex (40 mg, 0.17 mmol) were added and the solution was stirred at 90° C. for 16 h. The solution was cooled to room temperature before aq. NaHCO₃ was added and the aq. phase was extracted with DCM. The combined organic phase was washed with brine, dried over MgSO₄, filtered and the filtrate evaporated under reduced pressure affording the title compound which was used in the subsequent step without further purification. tert-Butyl-4-[5-(4-fluorophenyl)-7-vinyl-furo[3,2-b]pyridine-2-carbonyl]-3,3-dimethyl-piperazine-1-carboxylate (48 mg, 96% yield). ESI-MS m/z calc. 479.22203, found 480.03 (M+1)⁺; Retention time: 2.25 minutes using method C.

### Step II: tert-Butyl 4-[7-ethyl-5-(4-fluorophenyl)furo[3,2-b]pyridine-2-carbonyl]-3,3-dimethyl-piperazine-1-carboxylate

To a flask containing palladium hydroxide on carbon (7.0 mg, 0.0050 mmol) under a nitrogen atmosphere was added tert-butyl 4-[5-(4-fluorophenyl)-7-vinyl-furo[3,2-b]pyridine-2-carbonyl]-3,3-dimethyl-piperazine-1-carboxylate (48 mg, 0.10 mmol) in solution in EtOH (2 mL). The flask was evacuated under reduced pressure and re-filled with hydrogen 3 times, and the suspension was stirred under a hydrogen pressure at room temperature for 3 h. Hydrogen was evacuated and the flask backfilled with nitrogen. The catalyst was filtered on Celite® and washed with DCM and the filtrate was evaporated under reduced pressure affording the title compounds. tert-Butyl 4-[7-ethyl-5-(4-fluorophenyl)furo[3,2-b]pyridine-2-carbonyl]-3,3-dimethyl-piperazine-1-carboxylate (47 mg, 98% yield). ESI-MS m/z calc. 481.23768, found 482.83 (M+1)⁺; Retention time: 2.28 minutes using method C.

### Step III: (2,2-Dimethylpiperazin-1-yl)-[7-ethyl-5-(4-fluorophenyl)furo[3,2-b]pyridin-2-yl]methanone hydrochloride

The product was prepared according to General Procedure 2 using tert-butyl 4-[7-ethyl-5-(4-fluorophenyl)furo[3,2-b]pyridine-2-carbonyl]-3,3-dimethyl-piperazine-1-carboxylate (47 mg, 0.098 mmol), DCM (1 mL) and HCl in Dioxane (488 μL of 4 M, 1.95 mmol) affording the title compound. (2,2-Dimethylpiperazin-1-yl)-[7-ethyl-5-(4-fluorophenyl)furo[3,2-b]pyridin-2-yl]methanone hydrochloride (39 mg, 96% yield). ESI-MS m/z calc. 381.18526, found 382.41 (M+1)⁺; Retention time: 1.42 minutes using method C.

### Step IV: 1-[4-[5-(4-Fluorophenyl)-7-methyl-furo[3,2-b]pyridine-2-carbonyl]-3,3-dimethyl-piperazin-1-yl]-2-hydroxy-2-methyl-propan-1-one

The product was prepared according to General Procedure 3 using (2,2-dimethylpiperazin-1-yl)-[7-ethyl-5-(4-fluorophenyl)furo[3,2-b]pyridin-2-yl]methanone hydrochloride (39 mg, 0.093 mmol), HATU (50 mg, 0.13 mmol), DMF (439 μL), Hünig's base (98 μL, 0.56 mmol) and 2-hydroxy-2-methyl-propanoic acid (13 mg, 0.12 mmol) affording the title compound. 1-[4-[7-Ethyl-5-(4-fluorophenyl)furo[3,2-b]pyridine-2-carbonyl]-3,3-dimethyl-piperazin-1-yl]-2-hydroxy-2-methyl-propan-1-one (17 mg, 39% yield). ¹H NMR (400 MHz, DMSO-d6) δ 8.21-8.08 (m, 2H), 7.88 (s, 1H), 7.47 (s, 1H), 7.38-7.23 (m, 2H), 5.41 (s, 1H), 4.27-3.38 (m, 6H), 2.95 (q, J=7.7 Hz, 2H), 1.59-1.41 (m, 6H), 1.39-1.27 (m, 9H). ESI-MS m/z calc. 467.22205, found 468.52 (M+1)⁺; Retention time: 4.21 minutes using method D.

### Example 4: 1-[4-[7-Cyclopropyl-5-(4-fluorophenyl)furo[3,2-b]pyridine-2-carbonyl]-3,3-dimethyl-piperazin-1-yl]-2-hydroxy-2-methyl-propan-1-one

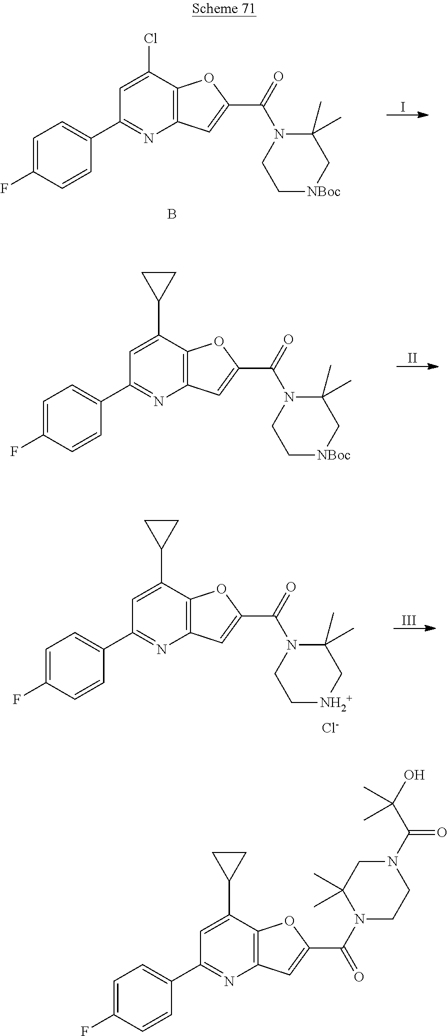

### Step I: tert-Butyl 4-[7-cyclopropyl-5-(4-fluorophenyl)furo[3,2-b]pyridine-2-carbonyl]-3,3-dimethyl-piperazine-1-carboxylate

To a round bottom flask containing a solution of Intermediate B (55 mg, 0.10 mmol) in dioxane (1.1 mL) was added Pd₂dba₃ (0.96 mg, 0.0010 mmol) and S-Phos (1.64 mg, 0.0040 mmol) and the solution was degassed with nitrogen for 5 minutes. K₃PO₄ (210 μL of 2 M. 0.42 mmol) and cyclopropylboronic acid (14 mg, 0.17 mmol) were added and the solution was stirred at 90° C. for 16 h. The solution was cooled to room temperature before aq. NaHCO₃ was added and the aq. phase was extracted with DCM. The combined organic phase was washed with brine, dried over MgSO₄ and filtered. The filtrate was evaporated under reduced pressure affording the title compound which was used in the subsequent step without further purification. tert-Butyl 4-[7-cyclopropyl-5-(4-fluorophenyl)furo[3,2-b]pyridine-2-carbonyl]-3,3-dimethyl-piperazine-1-carboxylate (47 mg, 91% yield). ESI-MS m/z calc. 493.23768, found 494.17 (M+1)⁺; Retention time: 2.28 minutes using method C.

### Step II: [7-Cyclopropyl-5-(4-fluorophenyl)furo[3,2-b]pyridin-2-yl]-(2,2-dimethylpiperazin-1-yl)methanone hydrochloride

The product was prepared according to General Procedure 2 using tert-butyl 4-[7-cyclopropyl-5-(4-fluorophenyl)furo[3,2-b]pyridine-2-carbonyl]-3,3-dimethyl-piperazine-1-carboxylate (47 mg, 0.095 mmol), DCM (1 mL) and HCl in Dioxane (476 μL of 4 M, 1.90 mmol) affording the title compound. [7-Cyclopropyl-5-(4-fluorophenyl)furo[3,2-b]pyridin-2-yl]-(2,2-dimethylpiperazin-1-yl)methanone hydrochloride (39 mg, 95% yield). ESI-MS m/z calc. 393.18526, found 394.40 (M+1)⁺; Retention time: 1.51 minutes using method C.

### Step III: 1-[4-[7-Cyclopropyl-5-(4-fluorophenyl)furo[3,2-b]pyridine-2-carbonyl]-3,3-dimethyl-piperazin-1-yl]-2-hydroxy-2-methyl-propan-1-one

The product was prepared according to General Procedure 3 using [7-cyclopropyl-5-(4-fluorophenyl)furo[3,2-b]pyridin-2-yl]-(2,2-dimethylpiperazin-1-yl)methanone hydrochloride (39 mg, 0.091 mmol), HATU (48 mg, 0.13 mmol), DMF (439 μL), Hünig's base (95 μL, 0.54 mmol) and 2-hydroxy-2-methyl-propanoic acid (12 mg, 0.12 mmol) affording the title compound. 1-[4-[7-Cyclopropyl-5-(4-fluorophenyl)furo[3,2-b]pyridine-2-carbonyl]-3,3-dimethyl-piperazin-1-yl]-2-hydroxy-2-methyl-propan-1-one (2.6 mg, 6% yield). ¹H NMR (400 MHz, DMSO-d6) δ 8.22-8.07 (m, 2H), 7.56 (s, 1H), 7.45 (s, 1H), 7.36-7.18 (m, 2H), 5.46-5.26 (m, 1H), 4.22-3.15 (m, 6H), 2.36-2.30 (m, 1H), 1.58-1.39 (m, 6H), 1.32 (s, 6H), 1.26-1.13 (m, 4H). ESI-MS m/z calc. 479.22205, found 480.32 (M+1)⁺; Retention time: 4.18 minutes using method D.

### Example 5: 4-[5-(4-Fluorophenyl)-7-isopropyl-furo[3,2-b]pyridine-2-carbonyl]-3,3-dimethyl-piperazin-2-one

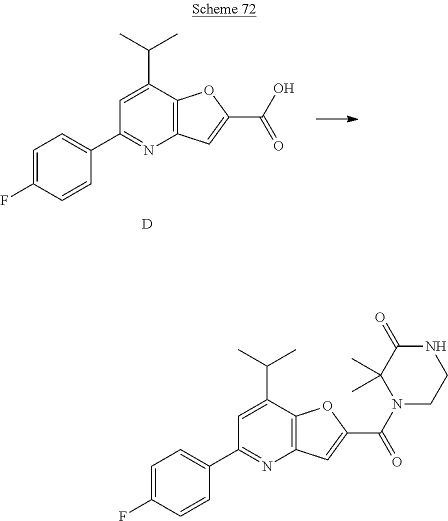

The product was prepared according to General Procedure 1 using Intermediate D (100 mg, 0.33 mmol), DMF (1.7 mL), Hünig's base (175 μL, 1.00 mmol), HATU (165 mg, 0.43 mmol) and 3,3-dimethylpiperazin-2-one (45 mg, 0.35 mmol) affording the title compound. 4-[5-(4-Fluorophenyl)-7-isopropyl-furo[3,2-b]pyridine-2-carbonyl]-3,3-dimethyl-piperazin-2-one (51 mg, 34% yield). ¹H NMR (400 MHz, DMSO-d6) δ 8.25-8.14 (m, 3H), 7.88 (s, 1H), 7.57 (s, 1H), 7.37-7.29 (m, 2H), 3.81-3.68 (m. 2H), 3.48-3.41 (m, 3H), 1.69 (s, 6H), 1.41 (d, J=6.9 Hz, 6H). ESI-MS m/z calc. 409.18017, found 410.69 (M+1)⁺; Retention time: 1.47 minutes using method C.

### Example 6: [5-(4-Fluorophenyl)-7-iso-propyl-furo[3,2-b]pyridin-2-yl]-[4-(1-hydroxycyclobutanecarbonyl)-2,2-dimethyl-piperazin-1-yl]methanone

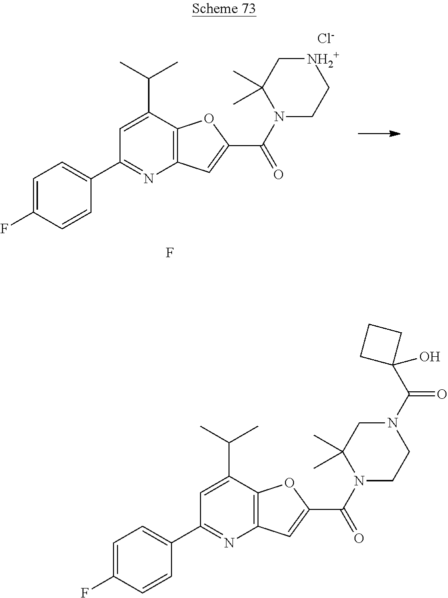

The product was prepared according to General Procedure 3 using Intermediate F (50 mg, 0.12 mmol), HATU (57 mg, 0.15 mmol), DMF (0.58 mL), Hünig's base (0.06 mL, 0.35 mmol) and 1-hydroxycyclobutanecarboxylic acid (14.1 mg, 0.12 mmol) affording the title compound. [5-(4-Fluorophenyl)-7-iso-propyl-furo[3,2-b]pyridin-2-yl]-[4-(1-hydroxycyclobutanecarbonyl)-2,2-dimethyl-piperazin-1-yl]methanone (12 mg, 20% yield). ¹H NMR (400 MHz, DMSO-d6) δ 8.21-8.16 (m, 2H), 7.88 (s, 1H), 7.51-7.49 (m, 1H), 7.37-7.29 (m, 2H), 6.03-5.95 (m, 1H), 3.96 (t, J=5.8 Hz, 1H), 3.81 (s, 1H), 3.74 (s, 1H), 3.62 (s, 1H), 3.53-3.47 (m, 1H), 3.47-3.38 (m, 1H), 2.62-2.54 (m, 3H), 2.11-1.98 (m, 2H), 1.85-1.71 (m, 1H), 1.55-1.46 (m, 7H), 1.42 (d, J=6.9 Hz, 6H). ESI-MS m/z calc. 493.23768, found 494.45 (M+1)⁺; Retention time: 1.62 minutes using method C.

### Example 7: 4-[5-(4-Fluorophenyl)-7-(1-methylcyclopropyl)furo[3,2-b]pyridine-2-carbonyl]-3,3-dimethyl-piperazin-2-one

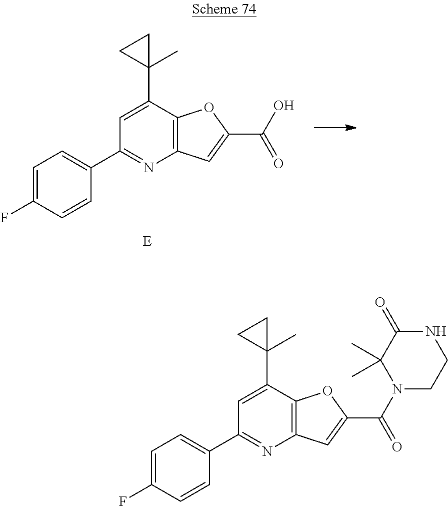

The product was prepared according to General Procedure 1 using Intermediate E (120 mg, 0.39 mmol), HATU (191 mg, 0.50 mmol). DMF (1.5 mL). Hünig's base (269 μL, 1.54 mmol) and 3,3-dimethylpiperazin-2-one (64 mg, 0.50 mmol) affording the title compound. 4-[5-(4-Fluorophenyl)-7-(1-methylcyclopropyl)furo[3,2-b]pyridine-2-carbonyl]-3,3-dimethyl-piperazin-2-one (58 mg, 35% yield). ¹H NMR (400 MHz, DMSO-d6) δ 8.21-8.09 (m, 3H), 7.74 (s, 1H), 7.53 (s, 1H), 7.34-7.23 (m, 2H), 3.78-3.65 (m, 2H), 1.66 (s, 6H), 1.53 (s, 3H), 1.28-1.21 (m, 2H), 0.93-0.83 (m, 2H). ESI-MS m/z calc. 421.18018, found 422.36 (M+1)+; Retention time: 1.49 minutes using method C.

### Example 8: [5-(4-Fluorophenyl)-7-(l-methylcyclopropyl)furo[3,2-b]pyridin-2-yl]-[4-(1-hydroxycyclopropanecarbonyl)-2,2-dimethyl-piperazin-1-yl]methanone

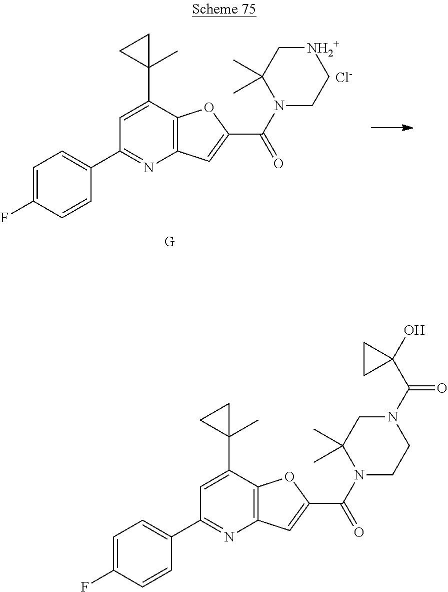

The product was prepared according to General Procedure 3 using Intermediate G (45 mg, 0.10 mmol), HATU (51 mg, 0.14 mmol), DMF (0.54 mL), Hünig's base (78 μL, 0.45 mmol) and 1-hydroxycyclopropane-1-carboxylic acid (200 μL of 0.5 M solution in NMP, 0.10 mmol) affording the title compound. [5-(4-Fluorophenyl)-7-(1-methylcyclopropyl)furo[3,2-b]pyridin-2-yl]-[4-(1-hydroxycyclopropanecarbonyl)-2,2-dimethyl-piperazin-1-yl]methanone (9.9 mg, 19% yield). ¹H NMR (400 MHz, DMSO-d6) δ 8.17-8.11 (m, 2H), 7.73 (s, 1H), 7.47 (s, 1H), 7.33-7.24 (m 2H), 6.34 (s, 1H), 4.26-3.34 (m, 5H), 1.54 (s, 3H), 1.49 (s, 6H), 1.30-1.23 (m, 2H), 0.97-0.74 (m, 6H). ESI-MS m/z calc. 491.22203, found 492.68 (M+1)⁺; Retention time: 1.55 minutes using method C.

### Example 9: [(3S)-4-(2,2-Difluorocyclopropanecarbonyl)-3-methyl-piperazin-1-yl]-[5-(4-fluorophenyl)-7-isopropyl-furo[3,2-b]pyridin-2-yl]methanone

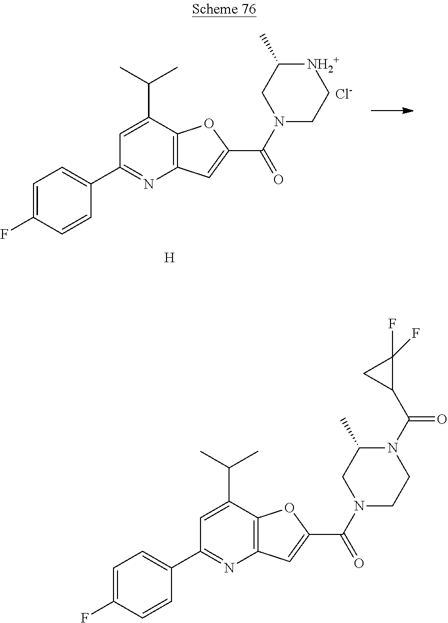

The product was prepared according to General Procedure 3 using Intermediate H (41 mg, 0.10 mmol), HATU (49 mg, 0.13 mmol), DMF (0.4 mL), Hünig's base (0.052 mL, 0.30 mmol) and 2,2-difluorocyclopropane-1-carboxylic acid (200 μL of 0.5 M solution in NMP, 0.10 mmol) affording the title compound. [(3S)-4-(2,2-Difluorocyclopropanecarbonyl)-3-methyl-piperazin-1-yl]-[5-(4-fluorophenyl)-7-isopropyl-furo[3,2-b]pyridin-2-yl]methanone (12.9 mg, 24% yield). ¹H NMR (400 MHz, DMSO-d6) δ 8.24-8.13 (m, 2H), 7.90 (s, 1H), 7.63 (s, 1H), 7.43-7.27 (m, 2H), 4.78-3.40 (m, 4H), 3.23-3.00 (m, 2H), 2.07-1.77 (m, 2H), 1.48-1.39 (m, 6H), 1.34-1.08 (m, 4H). ESI-MS m/z calc. 485.19263, found 486.75 (M+1)⁺, Retention time: 1.67 minutes using method C.

### Example 10: [5-(4-Fluorophenyl)-7-isopropyl-furo[3,2-b]pyridin-2-yl]-[4-(1-hydroxycyclobutanecarbonyl)piperazin-1-yl]methanone

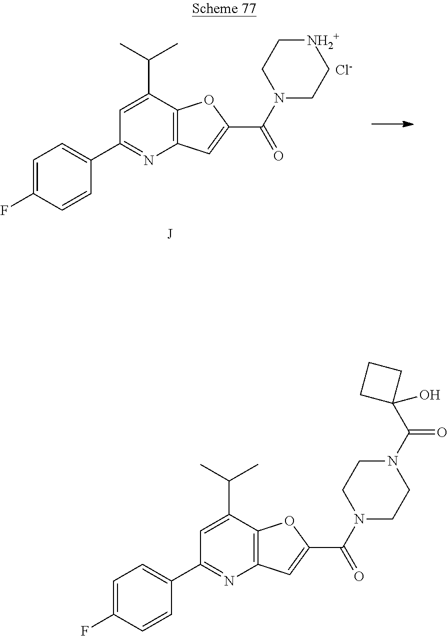

The product was prepared according to General Procedure 3 using Intermediate J (52 mg, 0.14 mmol). HATU (70 mg, 0.18 mmol), DMF (0.6 mL), Hünig's base (0.074 mL, 0.42 mmol) and 1-hydroxycyclobutanecarboxylic acid (18 mg, 0.16 mmol) affording the title compound. [5-(4-Fluorophenyl)-7-isopropyl-furo[3,2-b]pyridin-2-yl]-[4-(1-hydroxycyclobutanecarbonyl)piperazin-1-yl]methanone (2.9 mg, 4% yield). ¹H NMR (400 MHz, DMSO-d6) δ 8.22-8.16 (m, 2H), 7.89 (s, 1H), 7.59 (s, 1H), 7.38-7.29 (m 2H), 3.87-3.56 (m, 6H), 3.49-3.39 (m, 1H), 2.63-2.52 (m, 3H), 2.11-1.98 (m, 3H), 1.81-1.67 (m, 1H), 1.52-1.38 (m, 8H). ESI-MS m/z calc. 465.20638, found 466.69 (M+1)⁺; Retention time: 1.49 minutes using method C.

### Example 11: [5-(4-Fluorophenyl)-7-isopropyl-furo[3,2-b]pyridin-2-yl]-[(2R)-4-(1-hydroxycyclobutanecarbonyl)-2-methyl-piperazin-1-yl]methanone

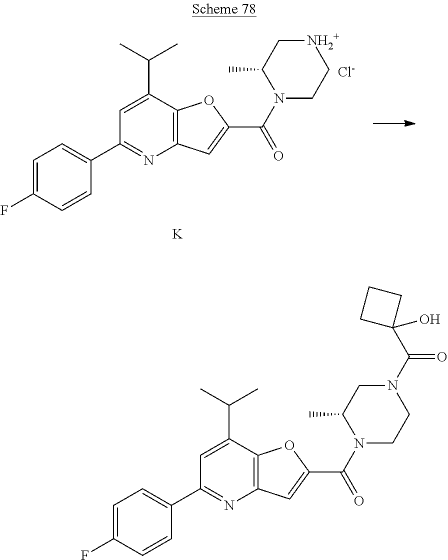

The product was prepared according to General Procedure 3 using Intermediate K (54 mg, 0.14 mmol), HATU (70 mg, 0.18 mmol), DMF (0.6 mL), Hünig's base (0.074 mL, 0.42 mmol) and 1-hydroxycyclobutanecarboxylic acid (18 mg, 0.16 mmol) affording the title compound. [5-(4-Fluorophenyl)-7-isopropyl-furo[3,2-b]pyridin-2-yl]-[(2R)-4-(1-hydroxycyclobutanecarbonyl)-2-methyl-piperazin-1-yl]methanone (12.1 mg, 18% yield). ¹H NMR (400 MHz, DMSO-d6) δ 8.23-8.15 (m, 2H), 7.89 (s, 1H), 7.59-7.55 (m, 1H), 7.37-7.29 (m, 2H), 6.20-5.91 (m, 1H), 4.84-4.46 (m, 1H), 4.41-4.08 (m, 2H), 4.06-3.57 (m, 2H), 3.52-3.27 (m, 1H), 3.24-2.56 (m, 2H), 2.18-1.95 (m, 2H), 1.84-1.68 (m, 1H), 1.55-1.37 (m 8H), 1.36-1.11 (m 4H). ESI-MS m/z calc. 479.22203, found 480.69 (M+1)⁺; Retention time: 1.54 minutes using method C.

### Example 12: [5-(4-Fluorophenyl)-7-isopropyl-furo[3,2-b]pyridin-2-yl]-[(2S)-4-(1-hydroxycyclobutanecarbonyl)-2-methyl-piperazin-1-yl]methanone

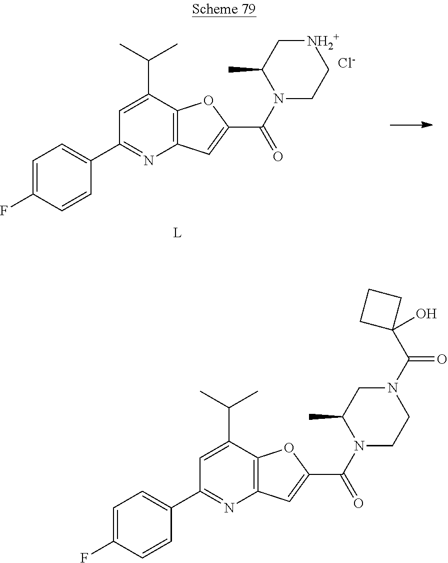 [SMILES: CC(C)c1cc(-c2ccc(F)cc2)nc2cc(C(=O)N3CC[NH2+]C[C@@H]3C)oc12 ; [Cl-] ; CC(C)c1cc(-c2ccc(F)cc2)nc2cc(C(=O)N3CCN(C(=O)C4(O)CCC4)C[C@@H]3C)oc12]

The product was prepared according to General Procedure 3 using Intermediate L (57 mg, 0.15 mmol), HATU (74 mg, 0.20 mmol), DMF (0.6 mL), Hünig's base (0.078 mL, 0.45 mmol) and 1-hydroxycyclobutanecarboxylic acid (19 mg, 0.17 mmol) affording the title compound. [5-(4-Fluorophenyl)-7-isopropyl-furo[3,2-b]pyridin-2-yl]-[(2S)-4-(1-hydroxycyclobutanecarbonyl)-2-methyl-piperazin-1-yl]methanone (7.2 mg, 10% yield). ¹H NMR (400 MHz, DMSO-d6) δ 8.23-8.15 (m, 2H), 7.89 (s, 1H), 7.59-7.55 (m, 1H), 7.38-7.29 (m, 2H), 4.86-3.85 (m, 5H), 3.52-3.28 (m, 1H), 3.22-3.05 (m, 1H), 2.96-2.87 (m, 1H), 2.73-2.58 (m, 1H), 2.48-2.41 (m, 1H), 2.17-1.92 (m, 2H), 1.84-1.68 (m, 1H), 1.54-1.37 (m, 7H), 1.35-1.15 (m, 3H). ESI-MS m/z calc. 479.22203, found 480.69 (M+1)⁺; Retention time: 1.55 minutes using method C.

### Example 13: (2,2-Dimethyl-4-propylsulfonyl-piperazin-1-yl)-[5-(4-fluorophenyl)-7-(1-methylcyclopropyl)furo[3,2-b]pyridin-2-yl]methanone

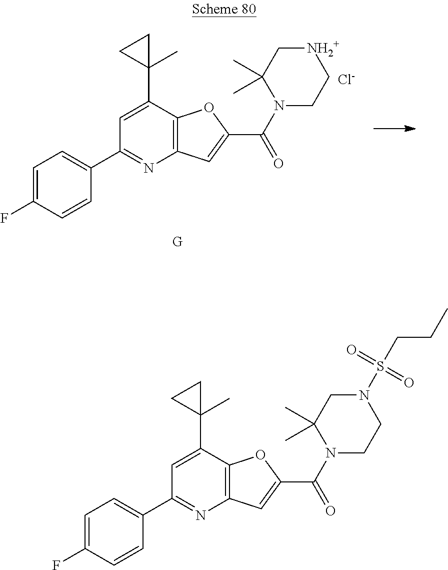

To a solution of Intermediate G (75 mg, 0.17 mmol) in DMF (845 μL) were added triethylamine (56 μL, 0.42 mmol) followed by propane-1-sulfonyl chloride (29 mg, 23 μL, 0.20 mmol) and the solution was stirred at room temperature for 16 h. The product was purified by mass-directed reverse phase HPLC affording the title compound. (2,2-Dimethyl-4-propylsulfonyl-piperazin-1-yl)-[5-(4-fluorophenyl)-7-(1-methylcyclopropyl)furo[3,2-b]pyridin-2-yl]methanone (9.0 mg, 10% yield). ¹H NMR (400 MHz, DMSO-d6) δ 8.20-8.09 (m, 2H), 7.74 (s, 1H), 7.49 (s, 1H), 7.36-7.23 (m, 2H), 3.79 (t, J=5.4 Hz, 2H), 3.48-3.41 (m, 2H), 3.27-3.23 (m, 2H), 3.14-3.06 (m, 2H), 1.75-1.61 (m, 2H), 1.54 (s, 3H), 1.49 (s, 6H), 1.30-1.22 (m, 2H), 0.97 (t, J=7.4 Hz, 3H), 0.93-0.86 (m, 2H). ESI-MS m/z calc. 513.2098, found 514.38 (M+1)⁺; Retention time: 4.35 minutes using method H.

### Example 14: Ethyl 4-[5-(4-fluorophenyl)-7-(1-methylcyclopropyl)furo[3,2-b]pyridine-2-carbonyl]-3,3-dimethyl-piperazine-1-carboxylate

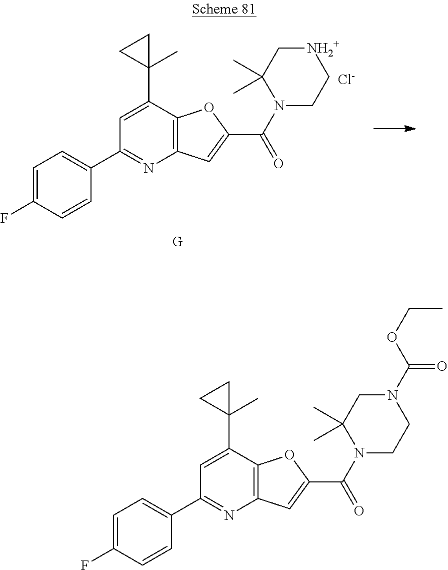

To a solution of Intermediate G (75 mg, 0.17 mmol) in DMF (845 μL) were added triethylamine (59 μL, 0.42 mmol) followed by ethyl chloroformate (22 mg, 19 μL, 0.20 mmol) and the solution was stirred at room temperature for 16 h. The product was purified by mass-directed reverse phase HPLC affording the title compound. Ethyl 4-[5-(4-fluorophenyl)-7-(1-methylcyclopropyl)furo[3,2-b]pyridine-2-carbonyl]-3,3-dimethyl-piperazine-1-carboxylate (5.7 mg, 7% yield). ¹H NMR (400 MHz, DMSO-d6) δ 8.21-8.07 (m, 2H), 7.73 (s, 1H), 7.46 (s, 1H), 7.34-7.23 (m, 2H), 4.06 (q, J=7.1 Hz, 2H), 3.85 (s, 2H), 3.52 (s, 2H), 1.54 (s, 3H), 1.47 (s, 6H), 1.31-1.21 (m, 2H), 1.17 (t, J=7.1 Hz, 3H), 0.93-0.84 (m, 2H). ESI-MS m/z calc. 479.22205, found 480.68 (M+1)⁺; Retention time: 3.70 minutes using method A.

### Example 15: [1-(2S)-4-[5-(4-Fluorophenyl)-7-(1-methylcyclopropyl)furo[3,2-b]pyridine-2-carbonyl]-2-methyl-piperazin-1-yl]-2-hydroxy-2-methyl-propan-1-one

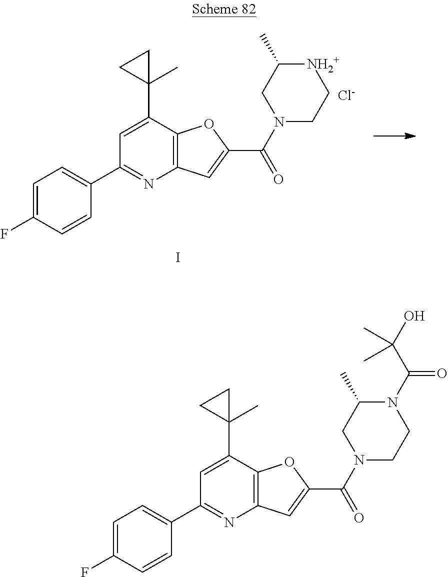

The product was prepared according to General Procedure 3 using Intermediate I (45 mg, 0.10 mmol), HATU (49 mg, 0.13 mmol), DMF (630 μL), Hünig's base (73 μL, 0.42 mmol) and 2-hydroxy-2-methyl-propanoic acid (200 μL of 0.5 M solution in NMP, 0.10 mmol) affording the title compound. [1-[(2S)-4-[5-(4-Fluorophenyl)-7-(1-methylcyclopropyl)furo[3,2-b]pyridine-2-carbonyl]-2-methyl-piperazin-1-yl]-2-hydroxy-2-methyl-propan-1-one (4.3 mg, 8% yield). ¹H NMR (400 MHz. Chloroform-d) δ 8.00-7.90 (m, 2H), 7.58 (s, 1H), 7.55 (s, 1H), 7.23-7.12 (m, 2H), 5.13-2.75 (m, 5H), 1.78-1.55 (m, 9H), 1.47-1.13 (m, 8H), 0.97 (s, 2H). ESI-MS m/z calc. 479.22203, found 480.38 (M+1)⁺; Retention time: 3.56 minutes using method C.

### Example 16: 4-[2-(4-Fluorophenyl)-4-isopropyl-furo[32-d]pyrimidine-6-carbonyl]-3,3-dimethyl-piperazin-2-one

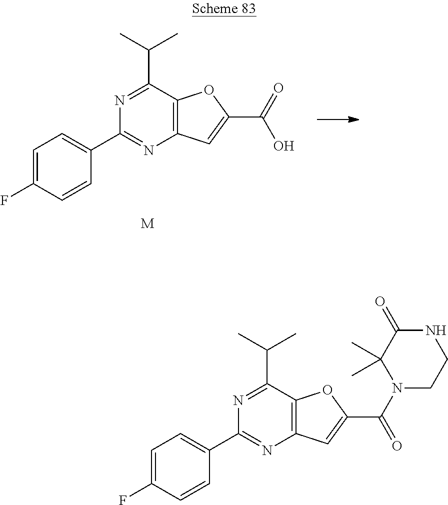

To a solution of Intermediate M (15 mg, 0.050 mmol) in DMF (1.5 mL) were added 3,3-dimethylpiperazin-2-one (9.6 mg, 0.075 mmol), HATU (29 mg, 0.075 mmol) and Hünig's base (18 μL, 0.10 mmol), and the solution was stirred at room temperature overnight. The volatiles were removed under reduced pressure and the residue was purified by flash chromatography on silica gel eluting with 50-100% EtOAc: Hexanes affording the title compound. 4-[2-(4-Fluorophenyl)-4-isopropyl-furo[3,2-d]pyrimidine-6-carbonyl]-3,3-dimethyl-piperazin-2-one (20 mg, 94% yield). ¹H NMR (400 MHz, Methanol-d4) δ 8.65-8.39 (m, 2H), 7.42 (s, 1H), 7.26-7.04 (m, 2H), 3.89-3.76 (m, 2H), 3.59 (p. J=6.9 Hz, 1H), 3.53-3.48 (m, 2H), 1.81 (s, 6H), 1.50 (d, J=6.9 Hz, 6H). ESI-MS m/z calc. 410.17542, found 411.14 (M+1)⁺; Retention time: 3.91 minutes using method A.

### Example 17: (2R)-1-[(2S)-4-[2-(4-Fluorophenyl)-4-isopropyl-furo[3,2-d]pyrimidine-6-carbonyl]-2-methyl-piperazin-1-yl]-2-hydroxy-4,4-dimethyl-pentan-1-one

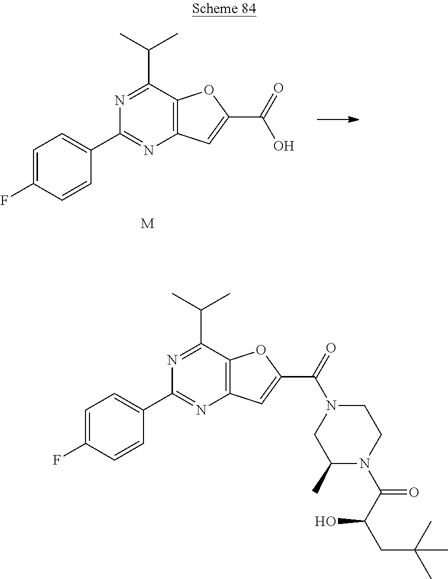

The product was prepared according to General Procedure 1, using Intermediate M (20 mg, 0.067 mmol), HATU (38 mg, 0.10 mmol), DMF (1.5 mL), Hünig's base (0.046 mL, 0.26 mmol) and Intermediate S (26 mg, 0.10 mmol) affording the title compound. (2R)-1-[(2S)-4-[2-(4-Fluorophenyl)-4-isopropyl-furo[3,2-d]pyrimidine-6-carbonyl]-2-methyl-piperazin-1-yl]-2-hydroxy-4,4-dimethyl-pentan-1-one (23 mg, 65% yield). ¹H NMR (400 MHz. Methanol-d4) δ 8.69-8.32 (m, 2H), 7.48 (s, 1H), 7.30-7.07 (m, 2H), 4.67-3.84 (m, 5H), 3.70-3.45 (m, 3H), 3.23-3.07 (m, 1H), 1.50 (dd, J=6.9, 2.0 Hz, 8H), 1.30 (dd, J=59.3, 6.7 Hz, 3H), 1.01 (s, 9H). ESI-MS m/z calc. 510.26423, found 511.52 (M+H)⁺; Retention time: 4.32 minutes using method A.

### Example 18: (2R)-1-[4-[2-(4-Fluorophenyl)-4-isopropyl-furo[3,2-d]pyrimidine-6-carbonyl]-3,3-dimethyl-piperazin-1-yl]-2-hydroxy-4,4-dimethyl-pentan-1-one

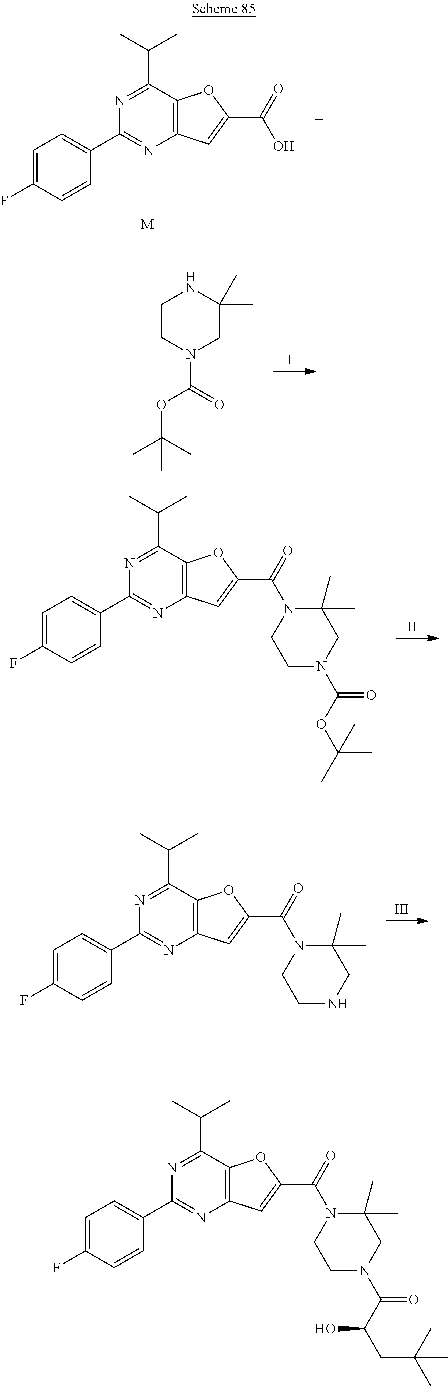

### Step I: tert-Butyl 4-[2-(4-Fluorophenyl)-4-isopropyl-furo[3,2-d]pyrimidine-6-carbonyl]-3,3-dimethyl-piperazine-1-carboxylate

To a solution of Intermediate M (27 mg, 0.090 mmol) in DMF (1.5 mL) were added tert-butyl 3,3-dimethylpiperazine-1-carboxylate (30 mg, 0.13 mmol), HATU (51 mg, 0.13 mmol) and Hünig's base (63 μL, 0.36 mmol) and the solution was stirred at room temperature overnight. The volatiles were removed under reduced pressure and the residue was purified by flash chromatography on silica gel eluting with 0-25% EtOAc: Hexanes affording the title compound. tert-Butyl 4-[2-(4-fluorophenyl)-4-isopropyl-furo[3,2-d]pyrimidine-6-carbonyl]-3,3-dimethyl-piperazine-1-carboxylate (32 mg, 72% yield). ¹H NMR (400 MHz, Chloroform-d) δ 8.63-8.37 (m, 2H), 7.27 (s, 1H), 7.19-6.95 (m, 2H), 3.79 (dd, J=6.6, 4.8 Hz, 2H), 3.66-3.40 (m, 5H), 1.59 (s, 6H), 1.47 (d, J=6.5 Hz, 15H).

### Step II: (2,2-Dimethylpiperazin-1-yl)-[2-(4-fluorophenyl)-4-isopropyl-furo[3,2-d]pyrimidin-6-yl]methanone hydrochloride

To the solution of tert-butyl 4-[2-(4-fluorophenyl)-4-isopropyl-furo[3,2-d]pyrimidine-6-carbonyl]-3,3-dimethyl-piperazine-1-carboxylate (32 mg, 0.064 mmol) in DCM (1 mL) was added a 4N HCl solution in 1,4-dioxane (0.50 mL, 2.00 mmol) and the solution was stirred at room temperature for 1 h. The volatiles were removed under reduced pressure affording the title compound. (2,2-Dimethylpiperazin-1-yl)-[2-(4-fluorophenyl)-4-isopropyl-furo[3,2-d]pyrimidin-6-yl]methanone hydrochloride (30 mg, 100% yield). ESI-MS m/z calc. 396.19615, found 397.59 (M+1)⁺; Retention time: 2.99 minutes using method A.

### Step III: (2R)-1-[4-[2-(4-Fluorophenyl)-4-isopropyl-furo[3,2-d]pyrimidine-6-carbonyl]-3,3-dimethyl-piperazin-1-yl]-2-hydroxy-4,4-dimethyl-pentan-1-one

To a solution of (2,2-dimethylpiperazin-1-yl)-[2-(4-fluorophenyl)-4-isopropyl-furo[3,2-d]pyrimidin-6-yl]methanone hydrochloride (30 mg, 0.069 mmol) in DMF (2 mL) were added (2R)-2-hydroxy-4,4-dimethyl-pentanoic acid (15 mg, 0.10 mmol), HATU (40 mg, 0.10 mmol) and Hünig's base (48 μL, 0.28 mmol) and the solution was stirred at room temperature overnight. The volatiles were removed under reduced pressure and the residue was purified by flash chromatography on silica gel eluting with 0-50% EtOAc: Hexanes affording the title compound. (2R)-1-[4-[2-(4-Fluorophenyl)-4-isopropyl-furo[3,2-d]pyrimidine-6-carbonyl]-3,3-dimethyl-piperazin-1-yl]-2-hydroxy-4,4-dimethyl-pentan-1-one (22 mg, 60% yield). ¹H NMR (400 MHz, Methanol-d4) δ 8.59-8.34 (m, 2H), 7.38 (d, J=3.6 Hz, 1H), 7.26-7.00 (m, 2H), 4.53 (ddd, J=9.7, 8.6, 3.2 Hz, 1H), 4.04-3.56 (m, 7H), 1.72-1.46 (m, 14H), 1.01 (d, J=4.1 Hz, 9H). ESI-MS m/z calc. 524.27988, found 525.55 (M+1)⁺; Retention time: 4.47 minutes using method A.

### Example 19: 4-(2-(4-Fluorophenyl)-4-(1-methylcyclopropyl)furo[3,2-d]pyrimidine-6-carbonyl)-3,3-dimethylpiperazin-2-one

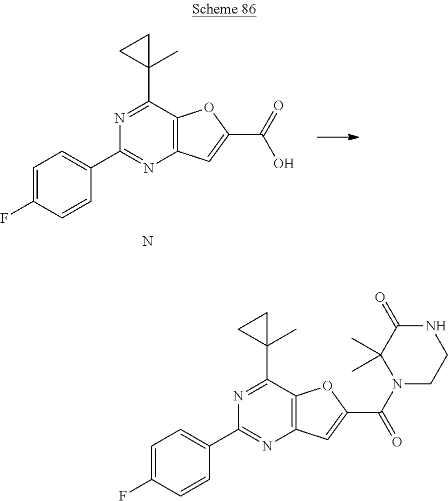

To a solution of Intermediate N (25 mg, 0.080 mmol) in DMF (1 mL) were added 3,3-dimethylpiperazin-2-one (15 mg, 0.12 mmol), HATU (46 mg, 0.12 mmol) and Hünig's base (56 μL, 0.32 mmol) and the solution was stirred at room temperature overnight. The volatiles were removed under reduced pressure and the residue was purified by flash chromatography on silica gel eluting with 50-100% EtOAc: Hexanes affording the title compound. 4-(2-(4-Fluorophenyl)-4-(1-methylcyclopropyl)furo[3,2-d]pyrimidine-6-carbonyl)-3,3-dimethylpiperazin-2-one (18 mg, 50% yield). ¹H NMR (400 MHz, Methanol-d4) δ 8.67-8.21 (m, 2H), 7.40 (s, 1H), 7.30-6.94 (m, 2H), 3.94-3.71 (m, 2H), 3.61-3.38 (m, 2H), 1.81 (s, 9H), 1.69 (q, J=3.9 Hz, 2H), 1.11-1.04 (m, 2H). ESI-MS m/z calc. 422.17542, found 423.35 (M+1)⁺; Retention time: 3.77 minutes using method A.

### Example 20: (R)-1-((S)-4-(2-(4-Fluorophenyl)-4-(1-methylcyclopropyl)furo[3,2-d]pyrimidine-6-carbonyl)-2-methylpiperazin-1-yl)-2-hydroxy-4,4-dimethylpentan-1-one

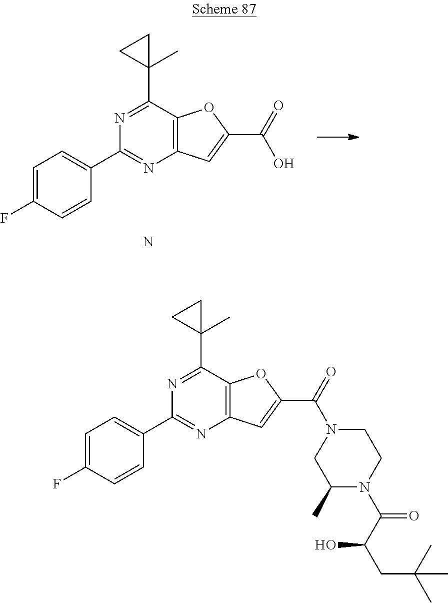

The product was prepared according to General Procedure 1 using Intermediate N (25 mg, 0.080 mmol), HATU (46 mg, 0.12 mmol), DMF (1 mL), Hünig's base (0.056 mL, 0.32 mmol) and Intermediate S (27 mg, 0.12 mmol) affording the title compound. (R)-1-((S)-4-(2-(4-Fluorophenyl)-4-(1-methylcyclopropyl)furo[3,2-d]pyrimidine-6-carbonyl)-2-methylpiperazin-1-yl)-2-hydroxy-4,4-dimethylpentan-1-one (28 mg, 67% yield). ¹H NMR (400 MHz, Methanol-d4) δ 8.55-8.35 (m, 2H), 7.47 (s, 1H), 7.27-7.09 (m, 2H), 4.48 (d, J=67.5 Hz, 3H), 3.96 (s, 1H), 3.80-3.43 (m, 1H), 3.28-3.02 (m, 3H), 1.82 (s, 3H), 1.73-1.67 (m, 2H), 1.60-1.44 (m, 2H), 1.35 (s, 1H), 1.20 (s, 2H), 1.10 (d, J=3.2 Hz; 2H), 1.04-0.98 (m, 9H). ESI-MS m/z calc. 522.26423, found 523.52 (M+1)⁺; Retention time: 4.63 minutes using method A.

### Example 21: (R)-1-(4-(2-(4-Fluorophenyl)-4-(1-methylcyclopropyl)furo[3,2-d]pyrimidine-6-carbonyl)-3,3-dimethylpiperazin-1-yl)-2-hydroxy-4,4-dimethylpentan-1-one

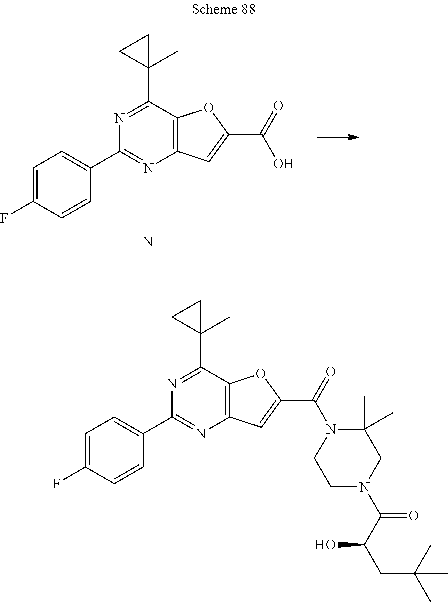

The product was prepared according to General Procedure 1 using Intermediate N (25 mg, 0.080 mmol), HATU (46 mg, 0.12 mmol), DMF (1 mL), Hünig's base (0.056 mL, 0.32 mmol) and Intermediate R (29 mg, 0.12 mmol) affording the title compound. (R)-1-(4-(2-(4-Fluorophenyl)-4-(1-methylcyclopropyl)furo[3,2-d]pyrimidine-6-carbonyl)-3,3-dimethylpiperazin-1-yl)-2-hydroxy-4,4-dimethylpentan-1-one (21 mg, 48% yield). ¹H NMR (400 MHz, Methanol-d4) δ 8.60-8.34 (m, 2H), 7.36 (d, J=3.7 Hz, 1H), 7.19 (t, J=8.8 Hz, 2H), 4.62-4.41 (m, 1H), 4.13-3.51 (m, 6H), 1.82 (d, J=1.7 Hz, 3H), 1.76-1.68 (m, 2H), 1.65-1.46 (m, 8H), 1.09 (q, J=3.7 Hz, 2H), 1.01 (d, J=5.6 Hz, 9H). ESI-MS m/z calc. 536.27988, found 537.17 (M+1)⁺; Retention time: 4.76 minutes using method A.

### Example 22: (R)-1-((S)-4-(5-(4-Fluorophenyl)-7-isopropylthiazolo[5,4-d]pyrimidine-2-carbonyl)-2-methylpiperazin-1-yl)-2-hydroxy-4,4-dimethylpentan-1-one

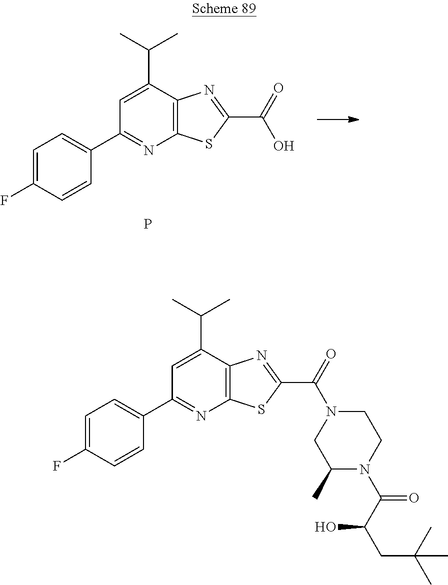

The product was prepared according to General Procedure 1 using Intermediate P (25 mg, 0.080 mmol), HATU (54 mg, 0.14 mmol), DMF (1 mL), Hünig's base (0.066 mL, 0.38 mmol) and Intermediate S (32 mg, 0.14 mmol) affording the title compound. (R)-1-((S)-4-(5-(4-Fluorophenyl)-7-isopropylthiazolo[5,4-d]pyrimidine-2-carbonyl)-2-methylpiperazin-1-yl)-2-hydroxy-4,4-dimethylpentan-1-one (26 mg, 47% yield). ¹H NMR (400 MHz, Methanol-d4) δ 8.10 (ddt, J=8.1, 5.2, 1.4 Hz 2H), 7.91-7.80 (m, 1H), 7.31-7.16 (m, 2H), 4.63-4.16 (m, 3H), 4.02-3.81 (m, 1H), 3.65-3.34 (m, 1H), 3.28-3.01 (m, 4H), 1.46-1.20 (m, 11H), 1.05-0.97 (m, 9H). ESI-MS m/z calc. 526.24139, found 527.44 (M+1)⁺; Retention time: 3.75 minutes using method A.

### Example 23: 4-(5-(4-Fluorophenyl)-7-isopropylthiazolo[5,4-d]pyrimidine-2-carbonyl)-3,3-dimethylpiperazin-2-one

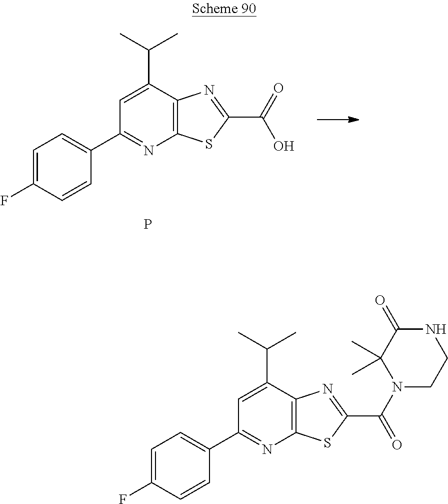

To a solution of Intermediate P (30 mg, 0.095 mmol) in DMF (1 mL) were added 3,3-dimethylpiperazin-2-one (18 mg, 0.14 mmol), HATU (54 mg, 0.14 mmol) and), Hünig's base (0.066 mL, 0.38 mmol) and the solution was stirred at room temperature overnight. The volatiles were removed under reduced pressure and the residue was purified by mass-directed reverse-phase HPLC affording the title compound. 4-(5-(4-Fluorophenyl)-7-isopropylthiazolo[5,4-d]pyrimidine-2-carbonyl)-3,3-dimethylpiperazin-2-one (4.9 mg, 1/1% yield). ESI-MS m/z calc. 426.15257, found 427.62 (M+1)⁺; Retention time: 3.46 minutes using method A.

### Example 24: (R)-1-(4-(5-(4-Fluorophenyl)-7-isopropylthiazolo[5,4-d]pyrimidine-2-carbonyl)-3,3-dimethylpiperazin-1-yl)-2-hydroxy-4,4-dimethylpentan-1-one

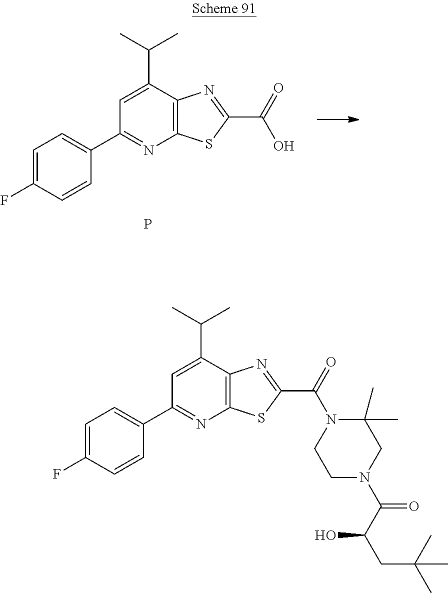

The product was prepared according to General Procedure 1 using Intermediate P (25 mg, 0.080 mmol), HATU (54 mg, 0.14 mmol), DMF (1 mL), Hünig's base (0.066 mL, 0.38 mmol) and Intermediate R (34 mg, 0.14 mmol) affording the title compound. (R)-1-((S)-4-(5-(4-Fluorophenyl)-7-isopropylthiazolo[5,4-d]pyrimidine-2-carbonyl)-2-methylpiperazin-1-yl)-2-hydroxy-4,4-dimethylpentan-1-one (3.4 mg, 6% yield). ESI-MS m/z calc. 540.25704, found 541.76 (M+1)⁺; Retention time: 4.37 minutes using method A.

### Example 25: 4-[5-(4-Fluorophenyl)-7-isopropyl-oxazolo[5,4-b]pyridine-2-carbonyl]-3,3-dimethyl-piperazin-2-one

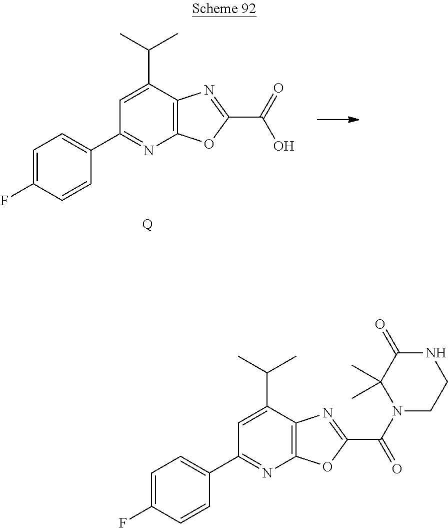

To a solution of Intermediate Q (30 mg, 0.098 mmol) in DCM (2 mL) was added a drop of DMF (10 μL) followed by oxalyl chloride (100 μL of 2 M in DCM. 0.20 mmol) dropwise and the solution was stirred at room temperature for 1 h. The volatiles were removed under reduced pressure and the residue was co-evaporated with DCM. The residue was dissolved in DCM (2 mL) and cooled to 0° C. before 3,3-dimethylpiperazin-2-one (19 mg, 0.15 mmol) and TEA (28 μL, 0.20 mmol) were added and the solution was stirred at room temperature for 1 h. The volatiles were removed under reduced pressure and the residue was purified by flash chromatography on silica gel eluting with 20-90% EtOAc: Hexanes affording the title compound. 4-[5-(4-Fluorophenyl)-7-isopropyl-oxazolo[5,4-b]pyridine-2-carbonyl]-3,3-dimethyl-piperazin-2-one (12 mg, 27% yield). ¹H NMR (400 MHz, Chloroform-d) δ 8.19-7.97 (m, 2H), 7.64 (s, 1H), 7.17 (t, J=8.5 Hz, 2H), 6.02 (s, 1H), 3.97 (t, J=4.8 Hz, 2H), 3.75-3.35 (m, 3H), 1.88 (s, 6H), 1.45 (d, J=6.9 Hz, 6H). ESI-MS m/z calc. 410.17542, found 411.31 (M+1)⁺; Retention time: 3.09 minutes using method A.

### Example 26: [5-(4-Fluorophenyl)-7-isopropyl-furo[3,2-b]pyridin-2-yl]-[4-(3-hydroxycyclobutanecarbonyl)-2,2-dimethyl-piperazin-1-yl]methanone

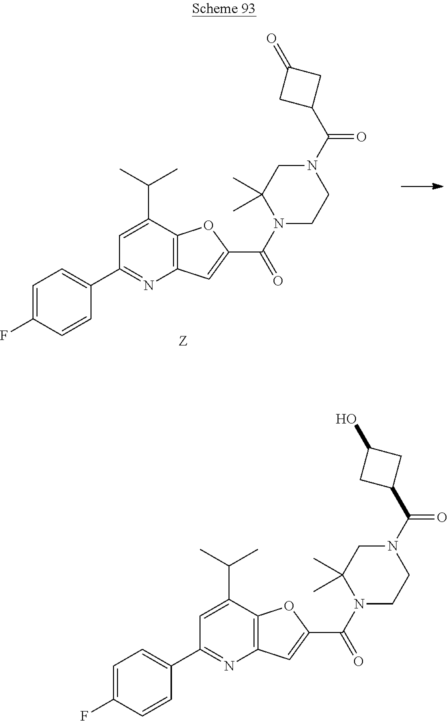

To a solution of Intermediate Z (166 mg, 0.34 mmol) in THF (901 μL) and MeOH (450 μL) at 0° C., was added NaBH₄ (14 mg, 0.37 mmol). The reaction was allowed to stir for 60 minutes, and then was quenched with water and extracted with EtOAc 3 times. The EtOAc layers were combined, dried over sodium sulfate, filtered and concentrated under reduced pressure. The crude product was purified by mass-directed reverse-phase HPLC affording the title compound. [5-(4-Fluorophenyl)-7-isopropyl-furo[3,2-b]pyridin-2-yl]-[4-(3-hydroxycyclobutanecarbonyl)-2,2-dimethyl-piperazin-1-yl]methanone (49 mg, 29% yield). ¹H NMR (400 MHz, DMSO-d6) δ 8.22-8.14 (m, 2H), 7.88 (d, J=2.0 Hz, 1H), 7.50 (d, J=10.1 Hz, 1H), 7.36-7.30 (m, 2H), 5.14-5.05 (m, 1H), 4.04-3.87 (m, 3H), 3.68-3.59 (m, 2H), 3.55 (s, 1H), 3.53-3.37 (m, 2H), 2.85-2.66 (m, 1H), 2.44-2.29 (m, 2H), 2.05-1.89 (m, 2H), 1.51-1.46 (m, 6H), 1.44-1.40 (m, 6H). ESI-MS m/z calc. 493.23767, found 494.69 (M+1)⁺; Retention time: 1.45 minutes using method C.

### Example 27: [5-(4-Fluorophenyl)-7-isopropyl-furo[3,2-b]pyridin-2-yl]-[4-(3-hydroxy-3-methyl-cyclobutanecarbonyl)-2,2-dimethyl-piperazin-1-yl]methanone

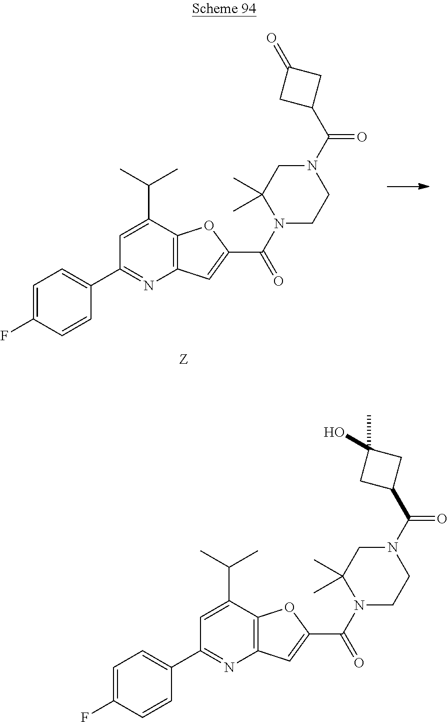

To a solution of Intermediate Z (166 mg, 0.34 mmol) in THF (3.38 mL) at −78° C., was added methyllithium (232 μL of 1.6 M in THF, 0.37 mmol). The reaction was cooled to 0° C. and quenched with a saturated aqueous solution of NH₄Cl. The aqueous layer was extracted with EtOAc 3 times. The combined organic extracts were dried over sodium sulfate, filtered and concentrated under reduced pressure. The crude product was purified by flash chromatography on silica gel eluting with 0-100% EtOAc: Hexanes, followed by mass-directed reverse-phase HPLC affording the title compound. [5-(4-Fluorophenyl)-7-isopropyl-furo[3,2-b]pyridin-2-yl]-[4-(3-hydroxy-3-methyl-cyclobutanecarbonyl)-2,2-dimethyl-piperazin-1-yl]methanone (6 mg, 3% yield). ¹H NMR (400 MHz, DMSO-d6) δ 8.20-8.10 (m, 2H), 7.85 (d, J=3.1 Hz, 1H), 7.48 (d, J=12.6 Hz, 1H), 7.36-7.24 (m, 2H), 3.88 (t, J=5.7 Hz, 2H), 3.66-3.56 (m, 2H), 3.53 (s, 1H), 3.50-3.32 (m, 2H), 2.92-2.76 (m, 1H), 2.21-1.99 (m, 4H), 1.46 (d, J=13.1 Hz, 6H), 1.39 (dd, J=6.9, 2.9 Hz, 6H), 1.25 (d, J=11.8 Hz, 3H). ESI-MS m/z calc. 507.25333, found 508.73 (M+1)⁺; Retention time: 1.52 minutes using method C.

### Example 28: [2,2-Dimethyl-4-(1-methylimidazol-4-yl)sulfonyl-piperazin-1-yl]-[5-(4-fluorophenyl)-7-isopropyl-furo[3,2-b]pyridin-2-yl]methanone

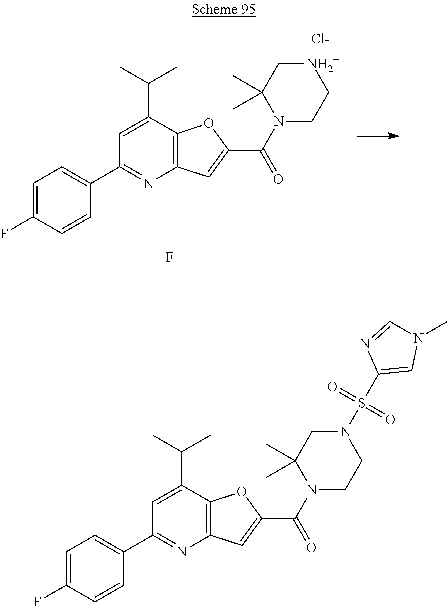

The product was prepared according to General Procedure 12 using Intermediate F (57 mg, 0.13 mmol) in DMF (0.66 mL), with triethylamine (46 μL, 0.33 mmol) and 1-methylimidazole-4-sulfonyl chloride (29 mg, 0.16 mmol). The reaction afforded the title compound. [2,2-Dimethyl-4-(1-methylimidazol-4-yl)sulfonyl-piperazin-1-yl]-[5-(4-fluorophenyl)-7-isopropyl-furo[3,2-b]pyridin-2-yl]methanone (25 mg, 35% yield). ¹H NMR (400 MHz, DMSO-d6) δ 8.21-8.14 (m, 2H), 7.90-7.86 (m, 3H), 7.51 (s. 1H), 7.37-7.29 (m, 2H), 3.77-3.70 (m, 5H), 3.43-3.35 (m, 3H), 3.12 (s, 2H), 1.53-1.48 (m, 6H), 1.43-1.36 (m, 6H). ESI-MS m/z calc. 539.20025, found 540.74 (M+1)⁺; Retention time: 1.78 minutes using method C.

### Example 29: [4-(2,3-Dimethylimidazol-4-yl)sulfonyl-2,2-dimethyl-piperazin-1-yl]-[5-(4-fluorophenyl)-7-isopropyl-furo[3,2-b]pyridin-2-yl]methanone

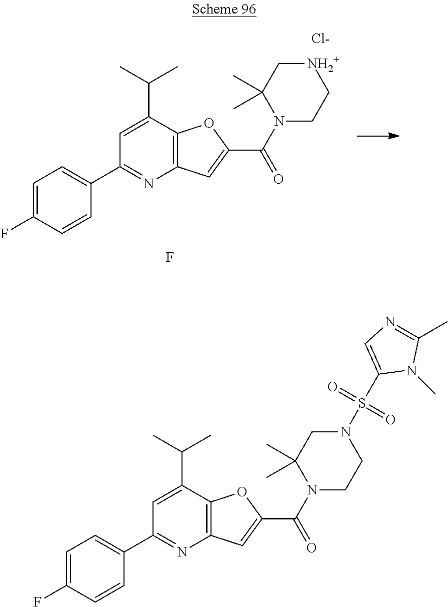

The product was prepared according to General Procedure 12 using Intermediate F (57 mg, 0.132 mmol) in DMF (0.66 mL), with triethylamine (46 μL, 0.33 mmol) and 2,3-dimethylimidazole-4-sulfonyl chloride (31 mg, 0.16 mmol). The reaction afforded the title compound. [4-(2,3-Dimethylimidazol-4-yl)sulfonyl-2,2-dimethyl-piperazin-1-yl]-[5-(4-fluorophenyl)-7-isopropyl-furo[3,2-b]pyridin-2-yl]methanone (21 mg, 27% yield). ¹H NMR (400 MHz, DMSO-d6) δ 8.23-8.13 (m, 2H), 7.88 (s, 1H), 7.53 (s, 1H), 7.51-7.47 (m, 1H), 7.33 (t, J=8.9 Hz, 2H), 3.82 (t, J=5.3 Hz, 2H), 3.70 (s, 3H), 3.42-3.32 (m, 3H), 3.25 (s, 2H), 2.39 (s, 3H), 1.55-1.46 (m, 6H), 1.44-1.35 (m, 6H). ESI-MS m/z calc. 553.21590, found 554.37 (M+1)⁺; Retention time: 1.71 minutes using method C.

### Example 30: 4-[7-tert-Butyl-5-(4-fluorophenyl)furo[3,2-b]pyridine-2-carbonyl]-3,3-dimethyl-piperazin-2-one

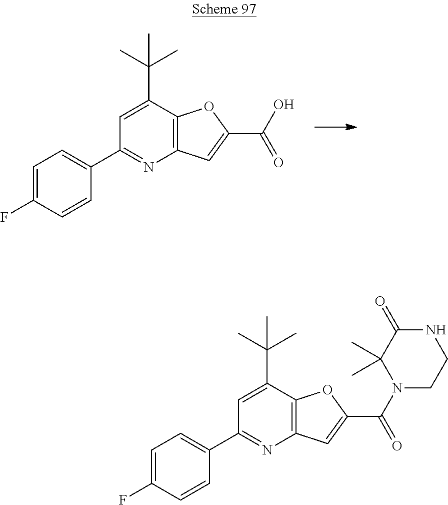 [SMILES: CC(C)(C)c1cc(-c2ccc(F)cc2)nc2cc(C(=O)O)oc12 ; CC(C)(C)c1cc(-c2ccc(F)cc2)nc2cc(C(=O)N3CCNC(=O)C3(C)C)oc12]

7-tert-Butyl-5-(4-fluorophenyl)furo[3,2-b]pyridine-2-carboxylic acid (50 mg, 0.16 mmol) and 3,3-dimethylpiperazin-2-one (23 mg, 0.18 mmol) were dissolved in DMF (0.53 mL) followed by Hünig's base (97 μL, 0.56 mmol) and HATU (73 mg, 0.19 mmol) and the resulting solution was stirred at room temperature for 1 h. Saturated aq. NH₄Cl. was added and the aqueous phase was extracted twice with EtOAc. The combined organic phases were washed with 1N HCl, sat. aq. NaHCO₃ and brine dried over Na₂SO₄ and filtered. The filtrate was evaporated under reduced pressure. The crude product was purified by flash chromatography on silica gel eluting with 50-100% EtOAc: Hexanes affording the title compound. 4-[5-(4-Fluorophenyl)-7-isopropyl-furo[3,2-b]pyridine-2-carbonyl]-3,3-dimethyl-piperazin-2-one (51 mg, 75% yield). ¹H NMR (400 MHz, Methanol-d4) δ 8.05-7.96 (m, 2H), 7.71 (s, 1H), 7.45 (s, 1H), 7.28-7.17 (m 2H), 3.90-3.84 (m, 2H), 3.56-3.49 (m, 2H), 2.79 (s, 1H), 1.82 (s, 6H), 1.57 (s, 9H). ESI-MS m/z calc. 423.1958, found 423.94 (M+1)⁺; Retention time: 1.67 minutes using method C.

### Example 31: [5-(4-Fluorophenyl)-7-(1-methylcyclopropyl)furo[3,2-b]pyridin-2-yl]-[4-(1-hydroxycyclopropanecarbonyl)-2,2-dimethyl-piperazin-1-yl]methanone

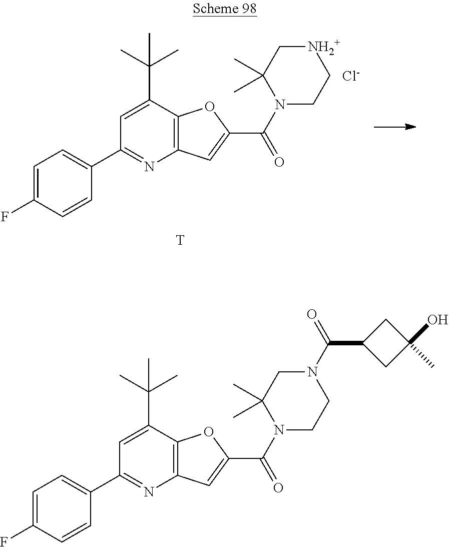

The product was prepared according to General Procedure 3 using Intermediate T (77 mg, 0.17 mmol), HATU (85 mg, 0.22 mmol), DMF (0.86 mL), Hünig's base (120 μL, 0.69 mmol) and 3-hydroxy-3-methyl-cyclobutanecarboxylic acid (27 mg, 0.21 mmol) affording the title compound. [7-tert-Butyl-5-(4-fluorophenyl)furo[3,2-b]pyridin-2-yl]-[4-(3-hydroxy-3-methyl-cyclobutanecarbonyl)-2,2-dimethyl-piperazin-1-yl]methanone (70 mg, 76% yield). ¹H NMR (400 MHz, DMSO-d6) δ 8.19-8.08 (m, 2H), 7.72 (d, J=2.6 Hz, 1H), 7.48 (d, J=11.7 Hz, 1H), 7.34-7.23 (m, 2H), 4.96 (d, J=18.8 Hz, 1H), 3.89 (s, 2H), 3.64-3.51 (m, 4H), 3.49-3.43 (m, 1H), 2.20-2.02 (m, 4H), 1.50 (d, J=3.1 Hz, 9H), 1.46 (d, J=12.8 Hz, 6H), 1.25 (d, J=12.0 Hz, 3H). ESI-MS m/z calc. 521.269, found 522.4 (M+1)⁺; Retention time: 1.7 minutes using method C.

### Example 32: 4-[7-iso-Propyl-5-[4-(trifluoromethyl)phenyl]furo[3,2-b]pyridine-2-carbonyl]-3,3-dimethyl-piperazin-2-one

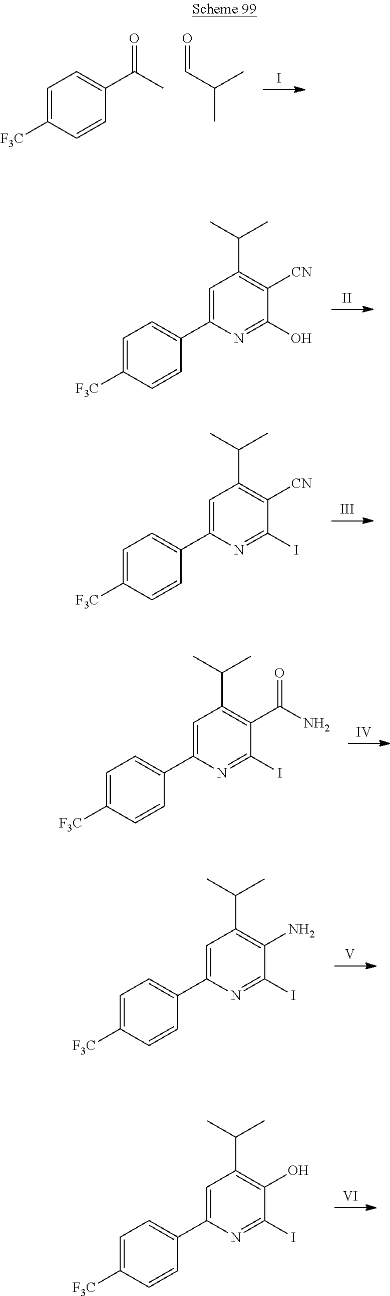

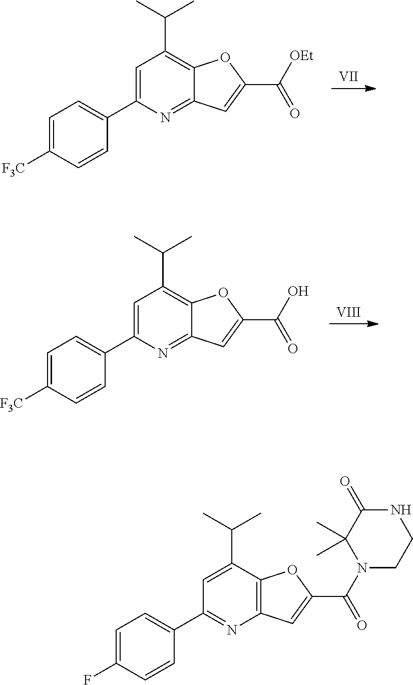

### Step I: 2-Hydroxy-4-isopropyl-6-[4-(trifluoromethyl)phenyl]pyridine-3-carbonitrile

The product was prepared according to General Procedure 4 using 1-[4-(trifluoromethyl)phenyl]ethanone (7.5 g, 39.86 mmol), isobutyraldehyde (3.47 mL, 38.02 mmol), ethyl 2-cyanoacetate (4.06 mL, 38.08 mmol), ethanol (50 mL) and ammonium acetate (27.9 g, 361.9 mmol) affording the title compound. 2-Hydroxy-4-isopropyl-6-[4-(trifluoromethyl)phenyl]pyridine-3-carbonitrile (6.96 g, 60% yield). ¹H NMR (400 MHz, Chloroform-d) δ 12.79 (s, 1H), 7.96 (d, J=8.3 Hz, 2H), 7.83 (d, J=8.7 Hz, 2H), 6.60 (s, 1H), 3.37 (p. J=6.9 Hz, 1H), 1.34 (d, J=6.9 Hz, 6H).

### Step II: 2-Iodo-4-isopropyl-6-[4-(trifluoromethyl)phenyl]pyridine-3-carbonitrile

The product was prepared according to General Procedure 5 using 2-hydroxy-4-isopropyl-6-[4-(trifluoromethyl)phenyl]pyridine-3-carbonitrile (8.48 g, 27.69 mmol), pyridine (2.58 mL, 31.90 mmol), acetonitrile (51 mL), triflic anhydride (5.13 mL, 30.49 mmol). NaI (14.53 g, 3.96 mL, 96.92 mmol) and HCl (2.54 mL of 12 M, 30.48 mmol) affording the title compound. 2-Iodo-4-isopropyl-6-[4-(trifluoromethyl)phenyl]pyridine-3-carbonitrile (4.97 g, 43% yield). ¹H NMR (400 MHz, Chloroform-d) δ 8.11 (d, J=8.1 Hz, 2H), 7.74 (d, J=8.1 Hz, 2H), 7.67 (s, 1H), 3.40 (dt, J=13.6, 6.6 Hz, 1H), 1.37 (d, J=6.8 Hz, 6H). ESI-MS m/z calc. 415.99973, found 417.21 (M+1)⁺; Retention time: 1.58 minutes using method I.

### Step III: 2-Iodo-4-isopropyl-6-[4-(trifluoromethyl)phenyl]pyridine-3-carboxamide

The product was prepared according to General Procedure 7 using 2-iodo-4-isopropyl-6-[4-(trifluoromethyl)phenyl]pyridine-3-carbonitrile (4.92 g, 11.82 mmol) and sulfuric acid (6 mL of 18 M. 108.0 mmol) affording the title compound. 2-Iodo-4-isopropyl-6-[4-(trifluoromethyl)phenyl]pyridine-3-carboxamide (4.65 g, 91% yield). ¹H NMR (400 MHz, Chloroform-d) δ 8.06 (d, J=8.1 Hz, 2H), 7.71 (d, J=8.0 Hz, 2H), 7.62 (s, 1H), 5.85 (d, J=81.7 Hz, 2H), 3.18 (p, J=7.1, 6.5 Hz, 1H), 1.31 (dd, J=6.8, 1.2 Hz, 6H). ESI-MS m/z calc. 434.01028, found 435.51 (M+1)⁺; Retention time: 0.81 minutes using method I.

### Step IV: 2-iodo-4-isopropyl-6-[4-(trifluoromethyl)phenyl]pyridin-3-amine

The product was prepared according to General Procedure 8 using KOH (4.21 g, 75.04 mmol), water (12 mL), bromine (720 μL, 13.98 mmol), 2-iodo-4-isopropyl-6-[4-(trifluoromethyl)phenyl]pyridine-3-carboxamide (4.65 g. 10.71 mmol), water (4 mL) and THF (4 mL) affording the title compound. 2-Iodo-4-isopropyl-6-[4-(trifluoromethyl)phenyl]pyridin-3-amine (4.02 g, 92% yield). ¹H NMR (400 MHz, Chloroform-d) δ 7.98 (d, J=8.7 Hz, 2H), 7.64 (d, J=8.7 Hz, 2H), 7.42 (s, 1H), 4.26 (s, 2H), 3.06-2.67 (m, 1H), 1.38 (d, J=6.9 Hz, 1H), 1.31 (d, J=6.8 Hz, 6H). ESI-MS m/z calc. 406.01538, found 407.26 (M+1)⁺; Retention time: 1.48 minutes using method I.

### Step V: 2-Iodo-4-isopropyl-6-[4-(trifluoromethyl)phenyl]pyridin-3-ol

The product was prepared according to General Procedure 9 using 2-iodo-4-isopropyl-6-[4-(trifluoromethyl)phenyl]pyridin-3-amine (4.02 g, 9.90 mmol), TFA (20 mL), isopentyl nitrite (8 mL, 59.55 mmol), MeOH (60 mL) and potassium carbonate (34 g, 246.0 mmol) affording the title compound. 2-Iodo-4-isopropyl-6-[4-(trifluoromethyl)phenyl]pyridin-3-ol (4.02 g, 100% yield). ¹H NMR (400 MHz, Chloroform-d) δ 7.99 (d, J=8.1 Hz, 2H), 7.67 (d, J=8.5 Hz, 2H), 7.47 (s, 1H), 3.54-3.12 (m, 1H), 1.29 (d, J=6.9 Hz, 6H). ESI-MS m/z calc. 406.9994, found 408.24 (M+1)⁺; Retention time: 1.41 minutes using method I.

### Step VI: Ethyl 7-isopropyl-5-[4-(trifluoromethyl)phenyl]furo[3,2-b]pyridine-2-carboxylate

The product was prepared according to General Procedure 10 using 2-iodo-4-isopropyl-6-[4-(trifluoromethyl)phenyl]pyridin-3-ol (4.02 g, 9.87 mmol), THF (22 mL), potassium carbonate (9.55 g, 69.10 mmol), PdCl₂(PPh₃)₂ (278 mg, 0.40 mmol). CuI (150 mg, 0.79 mmol), ethyl prop-2-ynoate (4 mL, 39.47 mmol) and THF (11 mL) affording the title compound. Ethyl 7-isopropyl-5-[4-(trifluoromethyl)phenyl]furo[3,2-b]pyridine-2-carboxylate (1.11 g. 30% yield). ¹H NMR (400 MHz, Chloroform-d) δ 8.11 (d, J=8.3 Hz, 2H), 7.73 (d, J=8.3 Hz, 2H), 7.69 (s, 1H), 7.65 (s, 1H), 4.47 (q, J=7.1 Hz, 2H), 3.57 (p, J=7.0 Hz, 1H), 1.47 (s, 3H), 1.45 (s, 6H). ESI-MS m/z calc. 377.12387, found 378.59 (M+1)⁺; Retention time: 1.64 minutes using method I.

### Step VII: 7-iso-Propyl-5-[4-(trifluoromethyl)phenyl]furo[3,2-b]pyridine-2-carboxylic acid

The product was prepared according to General Procedure 11 using ethyl 7-isopropyl-5-[4-(trifluoromethyl)phenyl]furo[3,2-b]pyridine-2-carboxylate (1.11 g, 2.94 mmol), dioxane (17 mL) and an aqueous solution of LiOH (2.94 mL of 2 M, 5.88 mmol) affording the title compound. 7-iso-Propyl-5-[4-(trifluoromethyl)phenyl]furo[3,2-b]pyridine-2-carboxylic acid (1.03 g, 88% yield). ¹H NMR (400 MHz, Chloroform-d) δ 8.11 (d, J=8.3 Hz, 2H), 7.85 (s, 1H), 7.75 (d, J=8.1 Hz, 2H), 7.68 (s, 1H), 3.58 (dt, J=13.7, 6.9 Hz, 1H), 1.47 (d, J=7.0 Hz, 6H). ESI-MS m/z calc. 349.0926, found 350.3 (M+1)⁺; Retention time: 1.28 minutes using method G.

### Step VIII: 4-[7-iso-Propyl-5-[4-(trifluoromethyl)phenyl]furo[3,2-b]pyridine-2-carbonyl]-3,3-dimethyl-piperazin-2-one

The product was prepared according to General Procedure 1 using 7-isopropyl-5-[4-(trifluoromethyl)phenyl]furo[3,2-b]pyridine-2-carboxylic acid (120 mg, 0.34 mmol), HATU (157 mg, 0.41 mmol), DMF (1.3 mL). Hünig's base (210 μL, 1.21 mmol) and 3,3-dimethylpiperazin-2-one (48 mg, 0.37 mmol) affording the title compound. 4-[7-iso-Propyl-5-[4-(trifluoromethyl)phenyl]furo[3,2-b]pyridine-2-carbonyl]-3,3-dimethyl-piperazin-2-one (130 mg, 80% yield). ¹H NMR (400 MHz, Chloroform-d) δ 8.10 (d, J=8.5 Hz, 2H), 7.73 (d, J=8.4 Hz, 2H), 7.61 (s, 1H), 7.49-7.42 (m, 1H), 6.03 (s, 1H), 3.86-3.80 (m, 2H), 3.63-3.56 (m, 2H), 3.44 (p, J=6.9 Hz, 1H), 1.87 (s, 6H), 1.46 (d, J=6.9 Hz, 6H). ESI-MS m/z calc. 459.17697, found 460.12 (M+1)⁺; Retention time: 0.99 minutes using method I.

### Example 33: 4-[7-tert-Butyl-5-[4-(trifluoromethyl)phenyl]furo[3,2-b]pyridine-2-carbonyl]-3,3-dimethyl-piperazin-2-one

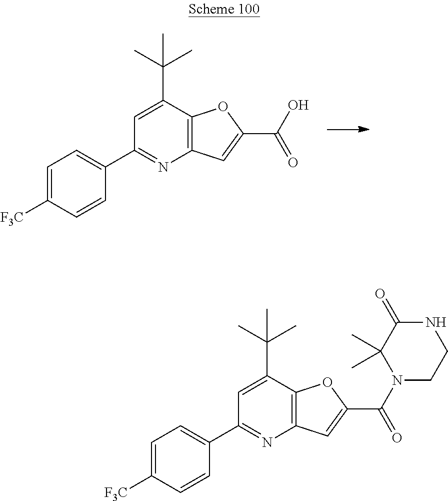 [SMILES: CC(C)(C)c1cc(-c2ccc(C(F)(F)F)cc2)nc2cc(C(=O)O)oc12 ; CC(C)(C)c1cc(-c2ccc(C(F)(F)F)cc2)nc2cc(C(=O)N3CCNC(=O)C3(C)C)oc12]

7-tert-Butyl-5-[4-(trifluoromethyl)phenyl]furo[3,2-b]pyridine-2-carboxylic acid (50 mg, 0.14 mmol) was dissolved in DMF (275 μL). DIPEA (84 μL, 0.48 mmol) and 3,3-dimethylpiperazin-2-one (17.64 mg, 0.14 mmol) were successively added at room temperature. After 2 minutes, HATU (68 mg, 0.18 mmol) was added and the reaction mixture was stirred overnight. Water is added and a solid precipitated. The heterogenous mixture was stirred for 1 h prior to filtration. The residue was purified by flash chromatography on silica gel eluting with 50-100% EtOAc: hexanes. The compound was then lyophilized to afford 4-[7-tert-butyl-5-[4-(trifluoromethyl)phenyl]furo[3,2-b]pyridine-2-carbonyl]-3,3-dimethyl-piperazin-2-one (45 mg, 68% yield). ¹H NMR (400 MHz, DMSO-d6) δ 8.37-8.28 (m, 2H), 8.17 (t, J=3.3 Hz, 1H), 7.90-7.78 (m, 3H), 7.59 (s, 1H), 3.81-3.69 (m, 2H), 3.42-3.33 (m, 2H), 1.67 (s, 6H), 1.51 (s, 9H). ESI-MS m/z calc. 473.19263, found 474.45 (M+1)⁺; Retention time: 1.07 minutes using method F.

### Example 34: 4-[7-tert-Butyl-5-(4-chlorophenyl)furo[3,2-b]pyridine-2-carbonyl]-3,3-dimethyl-piperazin-2-one

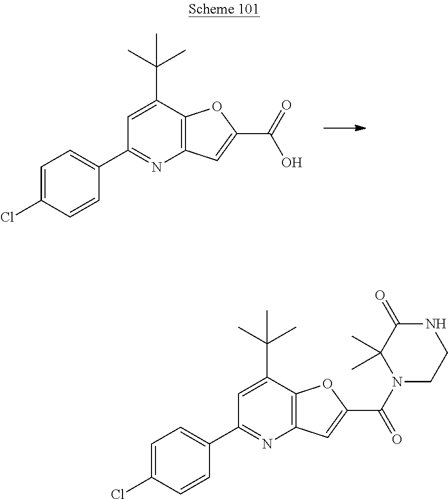 [SMILES: CC(C)(C)c1cc(-c2ccc(Cl)cc2)nc2cc(C(=O)O)oc12 ; CC(C)(C)c1cc(-c2ccc(Cl)cc2)nc2cc(C(=O)N3CCNC(=O)C3(C)C)oc12]

A protocol similar to General Procedure 1 was used. To a mixture of 7-tert-butyl-5-(4-chlorophenyl)furo[3,2-b]pyridine-2-carboxylic acid (75 mg, 0.23 mmol) 3,3-dimethylpiperazin-2-one (33 mg, 0.26 mmol) and (103 mg, 0.27 mmol) was added DMF (825 μL) then DIPEA (140 μL, 0.80 mmol). After stirring for 3 h, the reaction mixture was diluted with EtOAc (10 mL) and half-saturated aqueous NH₄Cl solution (5 mL). The layers were separated and the aqueous layer was first extracted with EtOAc (5 mL), and then extracted with a 4:1 mixture of CHCl₃/iPrOH (2×5 mL). The combined organic extracts were dried over MgSO₄, filtered, concentrated under reduced pressure. The residue was purified by flash chromatography on silica gel eluting with 50-100% EtOAc: Hexanes affording the title compound. The fractions were combined and concentrated, suspended in a H₂O/MeCN mixture (20% MeCN, 10 mL) with MeOH added (400 μL) and freeze-dried, providing the title compound (65.4 mg, 63% yield). ¹H NMR (400 MHz, DMSO-d6) δ 8.22-8.18 (m, 1H), 8.17-8.10 (m, 2H), 7.77 (s, 1H), 7.58 (s, 1H), 7.57-7.53 (m, 2H), 3.80-3.68 (m, 2H), 3.43-3.34 (m, 2H), 1.69 (s, 6H), 1.51 (s, 9H). ESI-MS m/z calc. 439.16626, found 440.41 (M+1)⁺; Retention time: 1.99 minutes using method C.

### Example 35: 4-(7-(tert-Butyl)-5-(4-fluoro-3-methylphenyl)furo[3,2-b]pyridine-2-carbonyl)-3,3-dimethylpiperazin-2-one

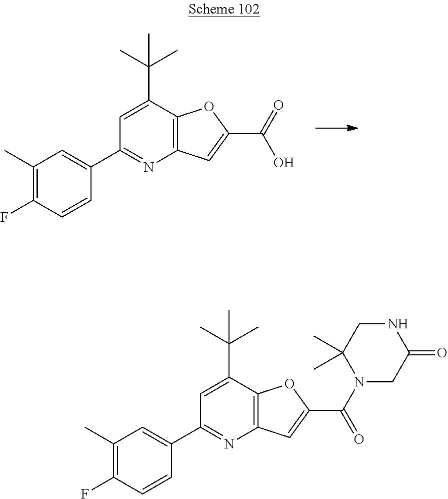 [SMILES: Cc1cc(-c2cc(C(C)(C)C)c3oc(C(=O)O)cc3n2)ccc1F ; Cc1cc(-c2cc(C(C)(C)C)c3oc(C(=O)N4CC(=O)NCC4(C)C)cc3n2)ccc1F]

7-tert-Butyl-5-(4-fluoro-3-methyl-phenyl)furo[3,2-b]pyridine-2-carboxylic acid (60 mg, 0.18 mmol) was dissolved in NMP (673 μL) before HATU (91 mg, 0.24 mmol), 3,3-dimethylpiperazin-2-one (28 mg, 0.22 mmol) and N-ethyl-N-isopropyl-propan-2-amine (128 μL, 0.73 mmol) were added. The solution was stirred at room temperature for 1 h before aq. NH₄Cl was added and the aq. phase was extracted twice with EtOAc. The combined organic phase was washed with sat. aq. NaHCO₃ and brine, dried over MgSO₄ and filtered. The filtrate was evaporated under reduced pressure. The residue was purified by flash chromatography on silica gel eluting with 0-15% MeOH: DCM affording the title compound. 4-(7-(tert-Butyl)-5-(4-fluoro-3-methylphenyl)furo[3,2-b]pyridine-2-carbonyl)-3,3-dimethylpiperazin-2-one (31 mg, 38% yield). ¹H NMR (400 MHz, DMSO-d6) δ 8.20 (t, J=3.2 Hz, 1H), 8.06 (ddd, J=7.7, 2.4, 0.9 Hz, 1H), 8.00-7.91 (m, 1H), 7.75 (s, 1H), 7.57 (s, 1H), 7.25 (dd, J=9.6, 8.6 Hz, 1H), 3.77 (dd, J=6.5, 3.4 Hz, 2H), 3.39 (dt, J=6.4, 3.6 Hz, 2H), 2.34 (d, J=1.9 Hz, 3H), 1.70 (s, 6H), 1.52 (s, 9H). ESI-MS m/z calc. 437.2115, found 438.13 (M+1)⁺; Retention time: 3.47 minutes using method A.

### Example 36: (7-(tert-Butyl)-5-(4-fluoro-3-methylphenyl)furo[3,2-b]pyridin-2-yl)(2,2-dimethyl-4-(1H-1,2,4-triazole-3-carbonyl)piperazin-1-yl)methanone

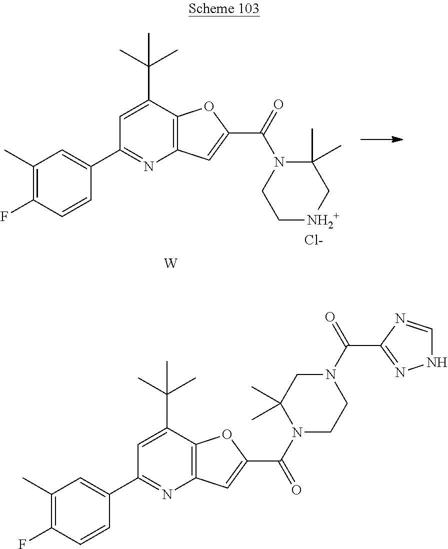

Intermediate W (75 mg, 0.15 mmol) was dissolved in NMP (841 μL) before HATU (75 mg, 0.20 mmol), 1H-1,2,4-triazole-3-carboxylic acid (21 mg, 0.18 mmol) and N-ethyl-N-isopropyl-propan-2-amine (105 μL, 0.60 mmol) were added. The solution was stirred at room temperature for 1 h before aq. NH₄Cl was added and the aq. phase was extracted twice with EtOAc. The combined organic phase was washed with sat. aq. NaHCO₃ and brine, dried over MgSO₄, filtered and the filtrate evaporated under reduced pressure. The residue was purified flash chromatography on silica gel eluting with 0-100% EtOAc: Hexanes affording the title compound. (7-(tert-Butyl)-5-(4-fluoro-3-methylphenyl)furo[3,2-b]pyridin-2-yl)(2,2-dimethyl-4-(1H-1,2,4-triazole-3-carbonyl)piperazin-1-yl)methanone (17 mg, 21% yield). ¹H NMR (400 MHz, DMSO-d6) δ 8.68 (s, 1H), 8.07-7.99 (m, 1H), 7.94 (ddd, J=8.0, 5.2, 2.4 Hz, 1H), 7.71 (d, J=8.5 Hz, 1H), 7.48 (d, J=9.8 Hz, 1H), 7.22 (td, J=9.1, 3.7 Hz, 1H), 4.48 (s, 1H), 4.10-3.61 (m, 6H), 2.31 (t, J=2.5 Hz, 3H), 1.61-1.39 (m, 15H). Signals doubled due to amide rotamers. ESI-MS m/z calc. 518.2442, found 519.09 (M+1)⁺; Retention time: 3.30 minutes using method A.

### Example 37: (7-(tert-Butyl)-5-(p-tolyl)furo[3,2-b]pyridin-2-yl)(4-((cis)-3-hydroxy-3-methylcyclobutane-1-carbonyl)-2,2-dimethylpiperazin-1-yl)methanone

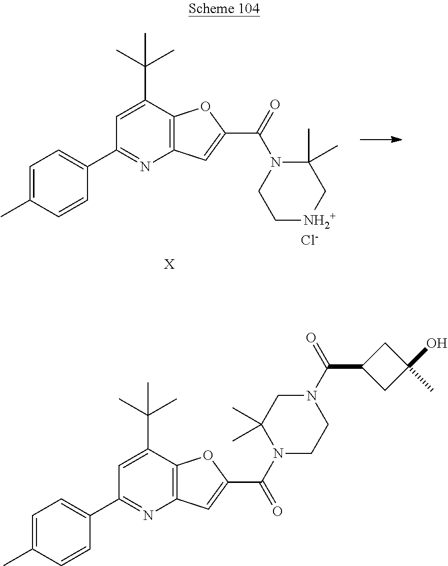

Intermediate X (60 mg, 0.13 mmol) was dissolved in NMP (673 μL) before HATU (62 mg, 0.16 mmol), 3-hydroxy-3-methyl-cyclobutanecarboxylic acid (20 mg, 0.15 mmol) and N-ethyl-N-isopropyl-propan-2-amine (87 μL, 0.50 mmol) were added. The solution was stirred at room temperature for 1 h before aq. NH₄Cl was added and the aq. phase was extracted twice with EtOAc. The combined organic phase was washed with sat. aq. NaHCO₃ and brine, dried over MgSO4, filtered and the filtrate evaporated under reduced pressure. The residue was by flash chromatography on silica gel affording the title compound. (7-(tert-Butyl)-5-(p-tolyl)furo[3,2-b]pyridin-2-yl)(4-((cis)-3-hydroxy-3-methylcyclobutane-1-carbonyl)-2,2-dimethylpiperazin-1-yl)methanone (42 mg, 59% yield). ESI-MS m/z calc. 517.2941, found 518.16 (M+1)⁺; Retention time: 3.44 minutes using method A.

### Example 38: 4-(7-(tert-Butyl)-5-(p-tolyl)furo[3,2-b]pyridine-2-carbonyl)-3,3-dimethylpiperazin-2-one

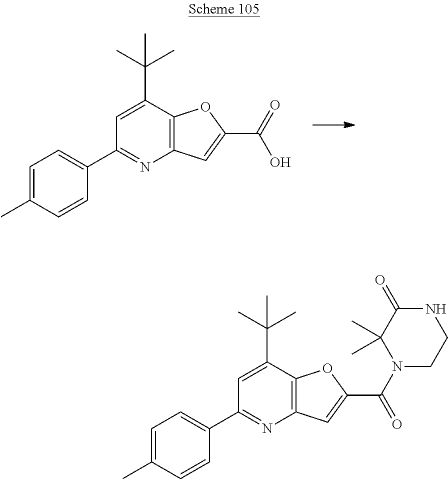 [SMILES: Cc1ccc(-c2cc(C(C)(C)C)c3oc(C(=O)O)cc3n2)cc1 ; Cc1ccc(-c2cc(C(C)(C)C)c3oc(C(=O)N4CCNC(=O)C4(C)C)cc3n2)cc1]

7-(tert-Butyl)-5-(p-tolyl)furo[3,2-b]pyridine-2-carboxylic acid (60 mg, 0.18 mmol) was dissolved in NMP (673 μL) before HATU (91 mg, 0.24 mmol), 3,3-dimethylpiperazin-2-one (28 mg, 0.22 mmol) and N-ethyl-N-isopropyl-propan-2-amine (128 μL, 0.73 mmol) were added. The solution was stirred at room temperature for 1 h before aq. NH₄Cl was added and the aq. phase was extracted twice with EtOAc. The combined organic phase was washed with sat. aq. NaHCO₃ and brine, dried over MgSO₄ and filtered. The filtrate was evaporated under reduced pressure. The residue was purified by flash chromatography on silica gel eluting with 0-100% EtOAc: Hexanes affording the title compound. 4-(7-(tert-Butyl)-5-(p-tolyl)furo[3,2-b]pyridine-2-carbonyl)-3,3-dimethylpiperazin-2-one (31 mg, 38% yield). ¹H NMR (400 MHz, DMSO-d6) δ 8.24-8.15 (m, 1H), 8.04-7.96 (m, 2H), 7.73 (s, 1H), 7.57 (s, 1H), 7.35-7.27 (m 2H), 3.82-3.73 (m, 2H), 3.40 (dd, J=6.1, 3.2 Hz, 2H), 2.37 (s, 3H), 1.70 (s, 6H), 1.52 (s, 9H). ESI-MS m/z calc. 418.2209, found 420.1 (M+1)⁺; Retention time: 3.36 minutes using method A.

### Example 39: 7-tert-Butyl-5-(4-chlorophenyl)furo[3,2-b]pyridin-2-yl]-[2,2-dimethyl-4-(1H-1,2,4-triazole-5-carbonyl)piperazin-1-yl]methanone

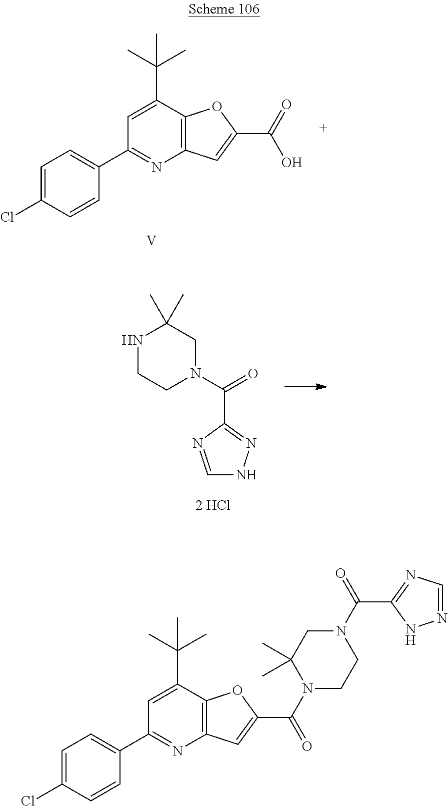

A protocol similar to General Procedure 1 was used. To a mixture of Intermediate V (100 mg, 0.30 mmol), (3,3-dimethylpiperazin-1-yl)-(1H-1,2,4-triazol-3-yl)methanone (Hydrochloric Acid (2)) (89 mg, 0.36 mmol) and HATU (152 mg, 0.40 mmol) was added DMF (1.10 mL) and DIPEA (240 μL, 1.38 mmol) and the reaction mixture was stirred overnight. Diluted with H₂O (4 mL), and extracted with EtOAc (5 mL, 2×3 mL). The combined organic extracts were washed sequentially with saturated aqueous NH₄Cl and brine (3 mL each), dried over MgSO₄, filtered and concentrated under reduced pressure. The residue was purified by flash chromatography on silica gel eluting with 0-10% MeOH: DCM, then purified again by reverse-phase flash chromatography on C18 support eluting with 10-90% MeCN: H₂O. The fractions were combined, concentrated to remove MeCN and freeze-dried, affording the title compound (75 mg, 45% yield) as a white solid. ¹H NMR (400 MHz, DMSO-d6) δ 8.53 (broad s, 1H), 8.22-8.08 (m, 2H), 7.77 (2s, 1H), 7.64-7.46 (m, 3H), 4.15-4.00 (m, 2H), 3.95 (broad s, 1H), 3.83 (s, 1H), 3.79-3.65 (m, 1H), 1.55 (2s, 9H), 1.48 (s, 6H). Signals doubled due to amide rotamers. ESI-MS m/z calc. 520.199, found 521.56 (M+1)⁺; Retention time: 0.93 minutes using method I.

### Example 40: [4-[7-tert-Butyl-5-[4-(trifluoromethyl)phenyl]furo[3,2-b]pyridine-2-carbonyl]-3,3-dimethyl-piperazin-1-yl]-(3-methyl-1H-pyrazol-5-yl)methanone

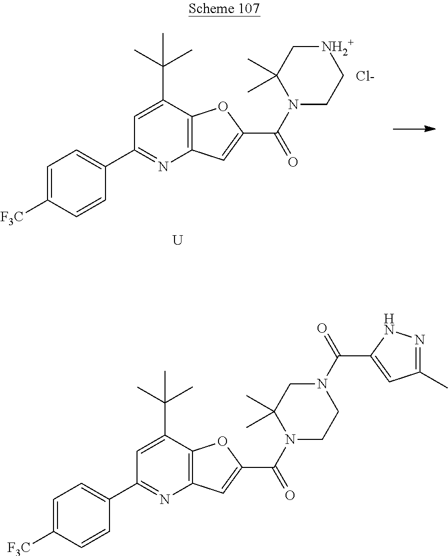

Intermediate U (100 mg, 0.20 mmol) was dissolved in DMF (550 μL). DIPEA (140 μL, 0.81 mmol) was added at room temperature, followed by 3-methyl-1H-pyrazole-5-carboxylic acid (28 mg, 0.22 mmol). After 2 minutes, HATU (100 mg, 0.26 mmol) was added and the reaction mixture was stirred overnight. The mixture was purified by mass-directed reverse phase HPLC to afford, upon lyophilization, [4-[7-tert-butyl-5-[4-(trifluoromethyl)phenyl]furo[3,2-b]pyridine-2-carbonyl]-3,3-dimethyl-piperazin-1-yl]-(3-methyl-1H-pyrazol-5-yl)methanone (53 mg, 44% yield). ¹H NMR (400 MHz, DMSO-d6) δ 8.42-8.24 (m, 2H), 7.96-7.71 (m, 3H), 7.53 (d, J=7.4 Hz, 1H), 6.36 (d, J=8.2 Hz, 1H), 4.25 (s, 1H), 4.09 (s, 1H), 4.00 (t, J=5.8 Hz, 1H), 3.91 (s, 1H), 3.77 (s, 1H), 3.64 (t, J=5.8 Hz, 1H), 2.23 (d, J=4.0 Hz, 3H), 1.52 (s, 8H), 1.49 (s, 4H), 1.45 (s, 4H). ESI-MS m/z calc. 567.24572, found 568.53 (M+1)⁺; Retention time: 1.11 minutes using method F.

### Example 41: 4-[7-tert-Butyl-5-(4-chloro-3-fluoro-phenyl)furo[3,2-b]pyridine-2-carbonyl]-3,3-dimethyl-piperazin-2-one

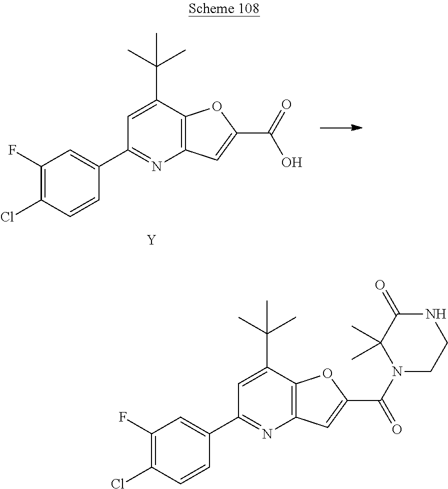

A protocol similar to General Procedure 1 was used. To a mixture of Intermediate Y (76 mg, 0.22 mmol), 3,3-dimethylpiperazin-2-one (32 mg, 0.25 mmol) and HATU (99 mg, 0.26 mmol) was added DMF (830 μL), and then DIPEA (132 μL, 0.76 mmol). After stirring for 1.5 h, the reaction mixture was diluted with H₂O (3 mL) and the precipitate was collected by filtration and washed with H₂O (2×0.5 mL). The solids were redissolved in DCM/MeOH and concentrated under reduced pressure. The residue was purified by flash chromatography on silica gel eluting with 50-100% EtOAc: Hexanes. The fractions were combined, concentrated under reduced pressure and suspended in a H₂O/MeCN mixture (20% MeCN) and freeze-dried, providing the title compound (64 mg, 64%6 yield). ¹H NMR (400 MHz, DMSO-d6) δ 8.23-8.13 (m, 2H), 8.03 (dd, J=8.4, 1.9 Hz, 1H), 7.83 (s, 1H), 7.71 (t, J=8.1 Hz, 1H), 7.59 (s, 1H), 3.82-3.71 (m, 2H), 3.45-3.34 (m, 2H), 1.69 (s, 6H), 1.52 (s, 9H). ¹⁹F NMR (376 MHz, DMSO-d6) δ −116.04 (dd, J=11.0, 7.8 Hz). ESI-MS m/z calc. 457.15686, found 458.44 (M+1)⁺; Retention time: 1.11 minutes using method I.

### Example 42: [7-tert-Butyl-5-(4-chlorophenyl)furo[3,2-b]pyridin-2-yl]-[2,2-dimethyl-4-(3-methyl-1H-1,2,4-triazole-5-carbonyl)piperazin-1-yl]methanone

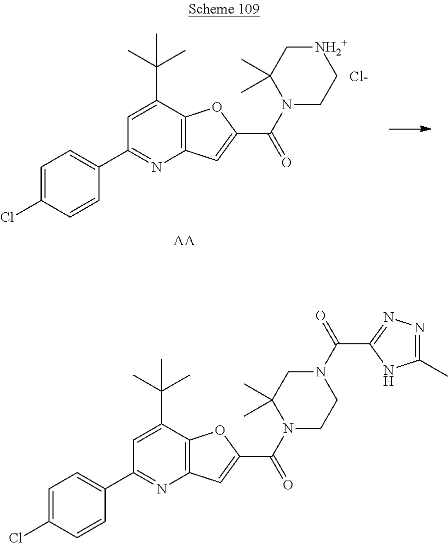 [SMILES: CC(C)(C)c1cc(-c2ccc(Cl)cc2)nc2cc(C(=O)N3CC[NH2+]CC3(C)C)oc12 ; [Cl-] ; "AA" ; Cc1nnc(C(=O)N2CCN(C(=O)c3cc4nc(-c5ccc(Cl)cc5)cc(C(C)(C)C)c4o3)C(C)(C)C2)[nH]1]

A protocol similar to General Procedure 3 was used. To a mixture of Intermediate AA (29 mg, 0.23 mmol) and HATU (89 mg, 0.23 mmol) were added DMF (1 mL) and DIPEA (52 μL, 0.30 mmol). After stirring for 5 min, Intermediate BB (100 mg, 0.20 mmol) was added followed by more DIPEA (69 μL, 0.40 mmol). The resulting mixture was stirred for 90 min, then diluted with H₂O (3 mL). The precipitated solid was collected by filtration and washed with H₂O (2×0.5 mL). It was then redissolved in a DCM/MeOH mixture, concentrated and purified by reverse-phase flash chromatography on C18 support eluting with 10-90% MeCN: H₂O. The fractions were combined, concentrated to remove the MeCN and freeze-dried to provide the title compound (61 mg, 55% yield). ¹H NMR (400 MHz, DMSO-d6) δ 8.25-8.07 (m, 2H), 7.76 (2s, 1H), 7.62-7.45 (m, 3H), 4.10-3.85 (m, 4H), 3.81 (s, 1H), 3.75-3.65 (m, 1H), 2.38 (2s, 3H), 1.54 (2s, 9H), 1.48 (2s, 6H). Signals doubled due to amide rotamers. ESI-MS m/z calc. 534.2146, found 535.52 (M+1)⁺; Retention time: 0.69 minutes using method I.

### Example 43: [7-tert-Butyl-5-(4-chloro-3-fluoro-phenyl)furo[3,2-b]pyridin-2-yl]-[2,2-dimethyl-4-(1H-1,2,4-triazole-5-carbonyl)piperazin-1-yl]methanone

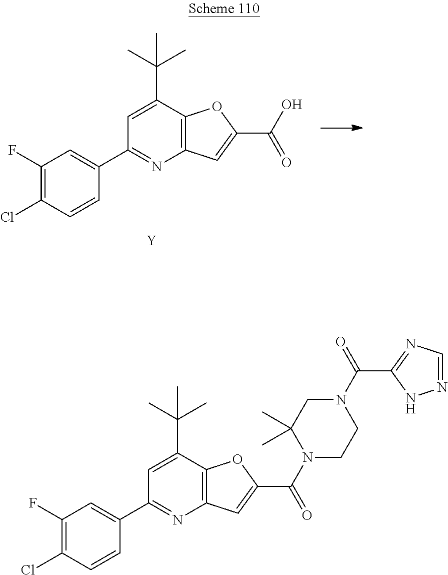

A protocol similar to General Procedure 1 was used. To a mixture of Intermediate Y (75 mg, 0.21 mmol), Intermediate BB (58 mg, 0.24 mmol) and HATU (104 mg, 0.27 mmol) was added DMF (820 μL) and DIPEA (168 μL, 0.96 mmol) and the mixture was stirred for 5 h at room temperature, then more 3,3-dimethylpiperazin-1-yl)-(1H-1,2,4-triazol-3-yl)methanone (23 mg) in DMF (0.4 mL) was added and the stirring was pursued overnight. The reaction mixture was diluted with H₂O (6 mL) and extracted with EtOAc (3×6 mL). The combined organic extracts were washed with saturated aqueous NH₄Cl (6 mL), brine (4 mL), dried over MgSO₄, filtered and concentrated under reduced pressure. The crude product was purified by reverse-phase flash chromatography on C18 support eluting with 10-90% MeCN: H₂O. The fractions were combined, concentrated to remove MeCN and freeze-dried, affording the title compound (43 mg. 36% yield). ¹H NMR (400 MHz, DMSO-d6) δ 8.22-8.10 (m, 1H), 8.07-7.96 (m, 1H), 7.82 (2s, 1H), 7.75-7.65 (m, 1H), 7.53 (2s, 1H), 4.13-3.89 (m, 4H), 3.83 (s, 1H), 3.79-3.68 (m, 1H), 1.55 (2s, 9H), 1.49 (s, 6H). Signals doubled due to amide rotamers. ¹⁹F NMR (376 MHz, DMSO-d6) δ −116.01-−116.10 (m). ESI-MS m/z calc. 538.1896, found 539.49 (M+1)⁺; Retention time: 0.77 minutes using method I.

### Example 44: 4-[5-(4-fluorophenyl)-7-sec-butyl-furo[3,2-b]pyridine-2-carbonyl]-3,3-dimethyl-piperazin-2-one

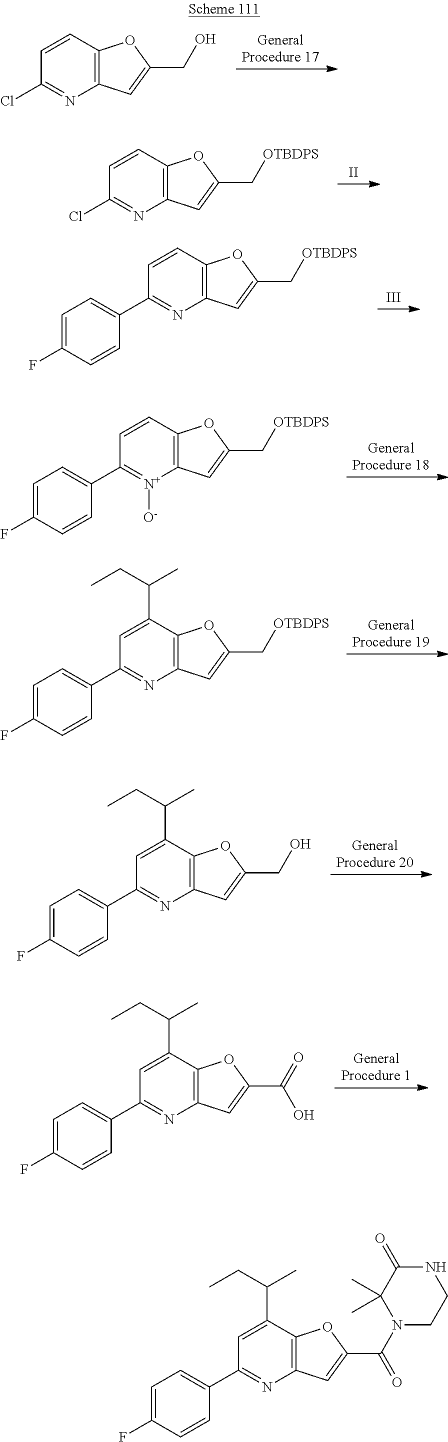

### Step I: tert-butyl-[(5-chlorofuro[3,2-b]pyridin-2-yl)methoxy]-diphenyl-silane

The product was prepared according to General Procedure 17 using (5-chlorofuro[3,2-b]pyridin-2-yl)methanol (12 g, 65.36 mmol), tert-butyl-chloro-diphenyl-silane (26.95 g, 25.50 mL, 98.04 mmol) and imidazole (11.09 g, 162.9 mmol) in DMF (109 mL) affording the title compound which was used in the subsequent step without further purification. tert-butyl-[(5-chlorofuro[3,2-b]pyridin-2-yl)methoxy]-diphenyl-silane (27.58 g, 65.36 mmol, 100%). ESI-MS m/z calc. 421.13, found 422.28 (M+1)⁺; Retention time: 2.34 minutes using method I.

### Step II: [5-(4-fluorophenyl)furo[3,2-b]pyridin-2-yl]methanol

To tert-butyl-[(5-chlorofuro[3,2-b]pyridin-2-yl)methoxy]-diphenyl-silane (2.28 g, 5.403 mmol) and (4-fluorophenyl)boronic acid (907.2 mg, 6.484 mmol) in 1,4-dioxane (10.81 mL) was added Na₂CO₃ (6.755 mL of 2 M, 13.51 mmol). Nitrogen was bubbled into the reaction mixture for 5 min before Pd(PPh₃)₄ (312.2 mg, 0.2702 mmol) was added and the mixture was stirred at 80° C. for 16 hrs. Water and EtOAc were added and the phases were separated. The aq. phase was washed twice with EtOAc and the combined organic phase dried over MgSO₄, filtered and the filtrate evaporated under reduced pressure. The residue was purified by silica gel chromatography affording the title compound. tert-butyl-[[5-(4-fluorophenyl)furo[3,2-b]pyridin-2-yl]methoxy]-diphenyl-silane (1.81 g, 3.758 mmol, 69.56%). ESI-MS m/z calc. 481.19, found 482.41 (M+1)⁺; Retention time: 1.97 minutes using method I.

### Step III: tert-butyl-[[5-(4-fluorophenyl)-4-oxido-furo[3,2-b]pyridin-4-ium-2-yl]methoxy]-diphenyl-silane

The product was prepared according to General Procedure 18 using tert-butyl-[[5-(4-fluorophenyl)furo[3,2-b]pyridin-2-yl]methoxy]-diphenyl-silane (1.81 g, 3.758 mmol) and m-CPBA (2.778 g, 11.27 mmol) in DCM (25.05 mL) affording the title compound. tert-butyl-[[5-(4-fluorophenyl)-4-oxido-furo[3,2-b]pyridin-4-ium-2-yl]methoxy]-diphenyl-silane (1.31 g, 2.632 mmol, 70.05%). ESI-MS m/z calc. 497.18, found 498.37 (M+1)⁺; Retention time: 1.41 minutes using method I.

### Step IV: tert-butyl-[[5-(4-fluorophenyl)-7-sec-butyl-furo[3,2-b]pyridin-2-yl]methoxy]-diphenyl-silane

The product was prepared according to General Procedure 19 using tert-butyl-[[5-(4-fluorophenyl)-4-oxido-furo[3,2-b]pyridin-4-ium-2-yl]methoxy]-diphenyl-silane (100 mg, 0.2010 mmol), iodocopper (15.31 mg, 0.08040 mmol), ethoxyethane (Boron Trifluoride (1)) (62.76 mg, 88.90 μL, 0.4422 mmol) and chloro(sec-butyl)magnesium (241.2 μL of 2 M. 0.4824 mmol) in THF (502.5 μL) affording the title compound which was used in the subsequent step without further purification. tert-butyl-[[5-(4-fluorophenyl)-7-sec-butyl-furo[3,2-b]pyridin-2-yl]methoxy]-diphenyl-silane (120 mg, 0.2232 mmol, 100.0%). ESI-MS m/z calc. 537.25, found 538.46 (M+1)⁺; Retention time: 2.34 minutes using method I.

### Step V: [5-(4-fluorophenyl)-7-sec-butyl-furo[3,2-b]pyridin-2-yl]methanol

The product was prepared according to General Procedure 20 using tert-butyl-[[5-(4-fluorophenyl)-7-sec-butyl-furo[3,2-b]pyridin-2-yl]methoxy]-diphenyl-silane (108 mg, 0.2008 mmol) and TBAF (401.6 μL of 1 M. 0.4016 mmol) in THF (542.7 μL) affording the title compound which was used in the subsequent step without further purification. [5-(4-fluorophenyl)-7-sec-butyl-furo[3,2-b]pyridin-2-yl]methanol (26 mg, 0.08686 mmol, 43.25%). ESI-MS m/z calc. 299.13, found 300.58 (M+1)⁺; Retention time: 2.34 minutes using method C.

### Step VI: 5-(4-fluorophenyl)-7-sec-butyl-furo[3,2-b]pyridine-2-carboxylic acid

The product was prepared according to General Procedure 21 using [5-(4-fluorophenyl)-7-sec-butyl-furo[3,2-b]pyridin-2-yl]methanol (26 mg, 0.08686 mmol), NMO monohydrate (133.0 mg, 0.8684 mmol) and TPAP (3.053 mg, 0.008686 mmol) in MeCN (363.2 μL) affording the title compound which was used in the subsequent step without further purification. 5-(4-fluorophenyl)-7-sec-butyl-furo[3,2-b]pyridine-2-carboxylic acid (10.6 mg, 0.03383 mmol, 38.94%). ESI-MS m/z calc. 313.11, found 314.38 (M+1)⁺; Retention time: 1.60 minutes using method C.

### Step VII: 4-[5-(4-fluorophenyl)-7-sec-butyl-furo[3,2-b]pyridine-2-carbonyl]-3,3-dimethyl-piperazin-2-one

The product was prepared according to General Procedure 1 using 5-(4-fluorophenyl)-7-sec-butyl-furo[3,2-b]pyridine-2-carboxylic acid (10.6 mg, 0.03383 mmol), 3,3-dimethylpiperazin-2-one (4.336 mg, 0.03383 mmol), HATU (16.72 mg, 0.04398 mmol) and DIPEA (15.30 mg, 20.62 μL, 0.1184 mmol) in DMF (54.06 μL) affording the title compound. 4-[5-(4-fluorophenyl)-7-sec-butyl-furo[3,2-b]pyridine-2-carbonyl]-3,3-dimethyl-piperazin-2-one (6.2 mg, 0.01458 mmol, 42.95%). ¹H NMR (400 MHz, DMSO-d6) δ 8.22-8.10 (m, 3H), 7.85 (s, 1H), 7.54 (s, 1H), 7.35-7.25 (m, 2H), 3.70 (dd, J=6.2, 3.5 Hz, 2H), 3.44-3.33 (m, 2H), 3.14 (q, J=7.1 Hz, 1H), 1.78 (ddq, J=20.5, 13.6, 7.2, 6.8 Hz, 2H), 1.66 (d, J=1.8 Hz, 6H), 1.37 (d, J=6.9 Hz, 3H), 0.81 (t, J=7.4 Hz, 3H). ESI-MS m/z found 424.29 (M+1)⁺; Retention time: 1.51 minutes using method C.

### Example 45: 4-(7-(tert-butyl)-5-(4-fluorophenyl)furo[3,2-b]pyridine-2-carbonyl)-1,3,3-trimethylpiperazin-2-one

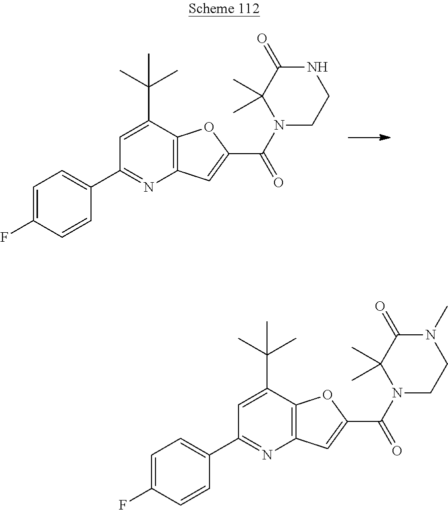 [SMILES: CC(C)(C)c1cc(-c2ccc(F)cc2)nc2cc(C(=O)N3CCNC(=O)C3(C)C)oc12 ; CN1CCN(C(=O)c2cc3nc(-c4ccc(F)cc4)cc(C(C)(C)C)c3o2)C(C)(C)C1=O]

4-(7-(tert-butyl)-5-(4-fluorophenyl)furo[3,2-b]pyridine-2-carbonyl)-3,3-dimethylpiperazin-2-one (50 mg, 0.1181 mmol) was dissolved in NMP (1.00 mL) followed by addition of 60% sodium hydride (5.7 mg, 0.1417 mmol) and the resulting mixture was stirred at room temperature for 15 minutes. MeI (63.8 mg, 36.8 μL, 0.5905 mmol) is added. The resulting mixture is stirred at 40° C. overnight. The crude product was purified by mass-directed reverse-phase HPLC affording the title compound4-(7-(tert-butyl)-5-(4-fluorophenyl)furo[3,2-b]pyridine-2-carbonyl)-1,3,3-trimethylpiperazin-2-one (17 mg, 32% yield). ¹H NMR (400 MHz, DMSO-d6) δ 8.17 (dd, J=8.9, 5.5 Hz, 2H), 7.76 (s, 1H), 7.59 (s, 1H), 7.33 (t, J=8.9 Hz, 2H), 3.83 (dd, J=6.9, 3.2 Hz, 2H), 3.59-3.52 (m, 2H), 2.94 (s, 3H), 1.71 (s, 6H), 1.53 (s, 9H). ESI-MS m/z found 438.08 (M+1)⁺; Retention time: 3.54 minutes using method A.

### Example 46: 2-[4-[7-tert-butyl-5-(4-fluorophenyl)furo[3,2-b]pyridine-2-carbonyl]-3,3-dimethyl-2-oxo-piperazin-1-yl]acetic acid

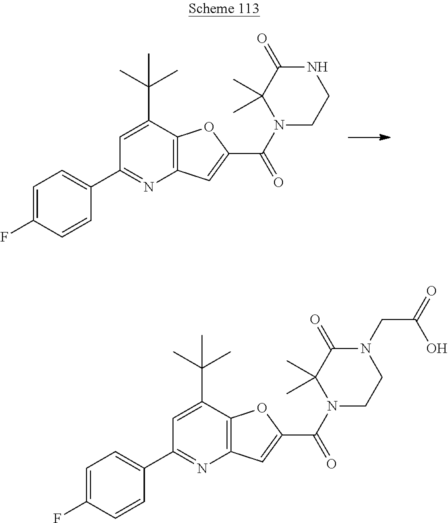 [SMILES: CC(C)(C)c1cc(-c2ccc(F)cc2)nc2cc(C(=O)N3CCNC(=O)C3(C)C)oc12 ; CC(C)(C)c1cc(-c2ccc(F)cc2)nc2cc(C(=O)N3CCN(CC(=O)O)C(=O)C3(C)C)oc12]

4-(7-(tert-butyl)-5-(4-fluorophenyl)furo[3,2-b]pyridine-2-carbonyl)-3,3-dimethylpiperazin-2-one (50 mg, 0.1181 mmol) was dissolved in NMP (1.00 mL) followed by addition of 60% sodium hydride (5.7 mg, 0.1417 mmol). The resulting mixture was stirred at room temperature for 15 minutes prior to tert-butyl 2-bromoacetate (23.04 mg, 0.1181 mmol) addition. It was then stirred at 40° C. for 18 hr. To the reaction mixture was added HCl 4M/dioxane (5 equiv.) and stirred at 40° C. for 72 hours. The wanted compound was purified by mass-directed reverse-phase HPLC affording the title compound 2-[4-[7-tert-butyl-5-(4-fluorophenyl)furo[3,2-b]pyridine-2-carbonyl]-3,3-dimethyl-2-oxo-piperazin-1-yl]acetic acid ¹H NMR (400 MHz, DMSO-d6) δ 12.82 (s, 1H), 8.17 (dd, J=8.9, 5.5 Hz, 2H), 7.76 (s, 1H), 7.60 (s, 1H), 7.33 (t, J=8.9 Hz, 2H), 4.10 (s, 2H), 3.88 (dd, J=6.6, 3.2 Hz, 2H), 3.61 (dd, J=6.4, 3.4 Hz, 2H), 1.72 (s, 6H), 1.53 (s, 9H). ESI-MS m/z found 482.09 (M+1)⁺; Retention time: 3.22 minutes using method A.

### Example 47: 4-(7-(tert-butyl)-5-(4-fluorophenyl)furo[3,2-b]pyridine-2-carbonyl)-1-(2-methoxyethyl)-3,3-dimethylpiperazin-2-one

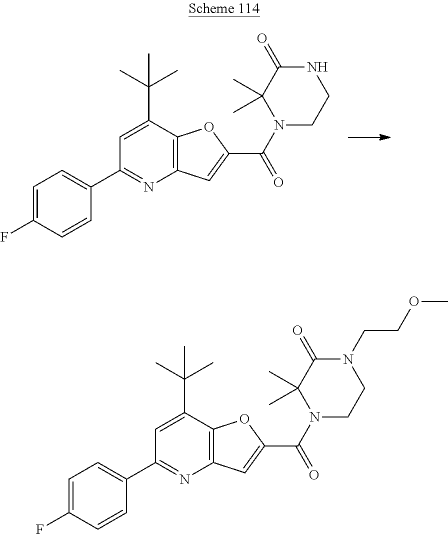 [SMILES: CC(C)(C)c1cc(-c2ccc(F)cc2)nc2cc(C(=O)N3CCNC(=O)C3(C)C)oc12 ; COCCN1CCN(C(=O)c2cc3nc(-c4ccc(F)cc4)cc(C(C)(C)C)c3o2)C(C)(C)C1=O]

4-(7-(tert-butyl)-5-(4-fluorophenyl)furo[3,2-b]pyridine-2-carbonyl)-3,3-dimethylpiperazin-2-one (50 mg, 0.1181 mmol) was dissolved in NMP (1.00 mL) followed by addition of 60% sodium hydride (5.7 mg, 0.1417 mmol) and the resulting mixture was stirred at room temperature for 15 minutes. 1-bromo-2-methoxy-ethane (82.1 mg, 55.5 μL, 0.5905 mmol) was added. The resulting mixture was stirred at 40 C for 1 hr. The crude product was purified by mass-directed reverse-phase HPLC affording the title compound 4-(7-(tert-butyl)-5-(4-fluorophenyl)furo[3,2-b]pyridine-2-carbonyl)-1-(2-methoxyethyl)-3,3-dimethylpiperazin-2-one (35 mg, 61% yield). ¹H NMR (400 MHz, DMSO-d6) δ 8.17 (dd, J=8.9, 5.5 Hz, 2H), 7.76 (s, 1H), 7.59 (s, 1H), 7.33 (t, J=8.9 Hz, 2H), 3.86-3.79 (min, 2H), 3.61 (dd, J=5.8, 3.9 Hz, 2H), 3.55 (dd, J=6.0, 4.2 Hz, 2H), 3.49 (td. J=5.2, 1.3 Hz, 2H), 3.27 (s, 3H), 1.71 (s, 6H), 1.53 (s, 9H). ESI-MS m/z found 482.13 (M+1)⁺; Retention time: 3.68 minutes using method A.

### Example 48: 4-[7-(4,4-difluorocyclohexyl)-5-(4-fluorophenyl)furo[3,2-b]pyridine-2-carbonyl]-3,3-dimethyl-piperazin-2-one

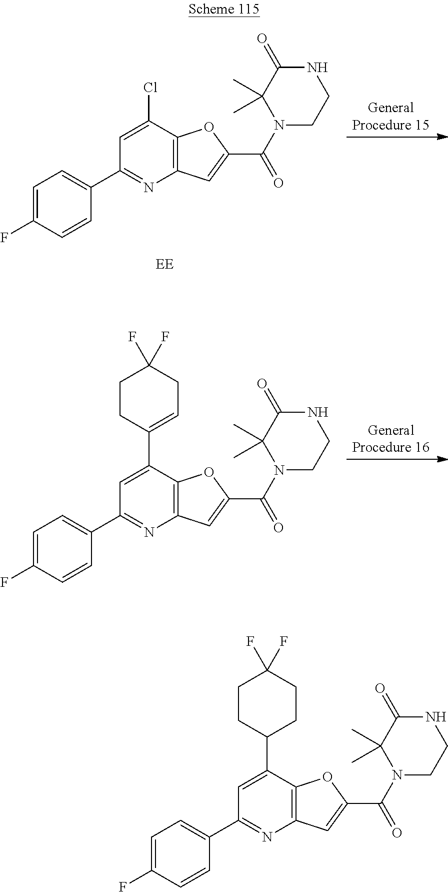 [SMILES: CC1(C)C(=O)NCCN1C(=O)c1cc2nc(-c3ccc(F)cc3)cc(Cl)c2o1 ; "GeneralProcedure 15" ; CC1(C)C(=O)NCCN1C(=O)c1cc2nc(-c3ccc(F)cc3)cc(C3=CCC(F)(F)CC3)c2o1 ; "GeneralProcedure 16" ; CC1(C)C(=O)NCCN1C(=O)c1cc2nc(-c3ccc(F)cc3)cc(C3CCC(F)(F)CC3)c2o1]

### Step I: 4-[7-(4,4-difluorocyclohexen-1-yl)-5-(4-fluorophenyl)furo[3,2-b]pyridine-2-carbonyl]-3,3-dimethyl-piperazin-2-one

The product was prepared according to General Procedure 15 using 4-[7-chloro-5-(4-fluorophenyl)furo[3,2-b]pyridine-2-carbonyl]-3,3-dimethyl-piperazin-2-one (Intermediate EE) (92 mg, 0.2290 mmol), Pd₂dba₃ (2.1 mg, 0.0023 mmol). S-Phos (3.6 mg, 0.0087 mmol), 2-(4,4-difluorocyclohexen-1-yl)-4,4,5,5-tetramethyl-1,3,2-dioxaborolane (111.8 mg, 0.46 mmol), 1,4-dioxane (1.9 mL) and K₃PO₄ (458.0 μL of 2 M, 0.92 mmol) affording the title compound. 4-[7-(4,4-difluorocyclohexen-1-yl)-5-(4-fluorophenyl)furo[3,2-b]pyridine-2-carbonyl]-3,3-dimethyl-piperazin-2-one (110 mg, 0.23 mmol, 99%) ESI-MS m/z calc. 483.17697, found 484.37 (M+1)⁺; Retention time: 1.66 minutes using method C.

### Step II: 4-[7-(4,4-difluorocyclohexyl)-5-(4-fluorophenyl)furo[3,2-b]pyridine-2-carbonyl]-3,3-dimethyl-piperazin-2-one

The product was prepared according to General Procedure 16 using 4-[7-(4,4-difluorocyclohexen-1-yl)-5-(4-fluorophenyl)furo[3,2-b]pyridine-2-carbonyl]-3,3-dimethyl-piperazin-2-one (110 mg, 0.23 mmol), PtO₂ (10 mg, 0.046 mmol) and methanol (4.6 mL) affording the title compound after mass-directed reverse phase HPLC. 4-[7-(4.4-difluorocyclohexyl)-5-(4-fluorophenyl)furo[3,2-b]pyridine-2-carbonyl]-3,3-dimethyl-piperazin-2-one (34 mg, 0.070 mmol, 31%). ¹H NMR (400 MHz, DMSO-d6) δ 8.24-8.16 (m, 3H), 7.91 (s, 1H), 7.59 (s, 1H), 7.36-7.27 (m, 2H), 3.78-3.71 (m, 2H), 3.40 (s, 2H), 3.29-3.21 (m, 1H), 2.24-1.97 (m, 8H), 1.69 (s, 6H). ESI-MS m/z calc. 485.19263, found 486.01 (M+1)⁺; Retention time: 3.28 minutes A.

### Example 49: 4-[7-tert-butyl-5-(4,4-difluorocyclohexyl)furo[3,2-b]pyridine-2-carbonyl]-3,3-dimethyl-piperazin-2-one

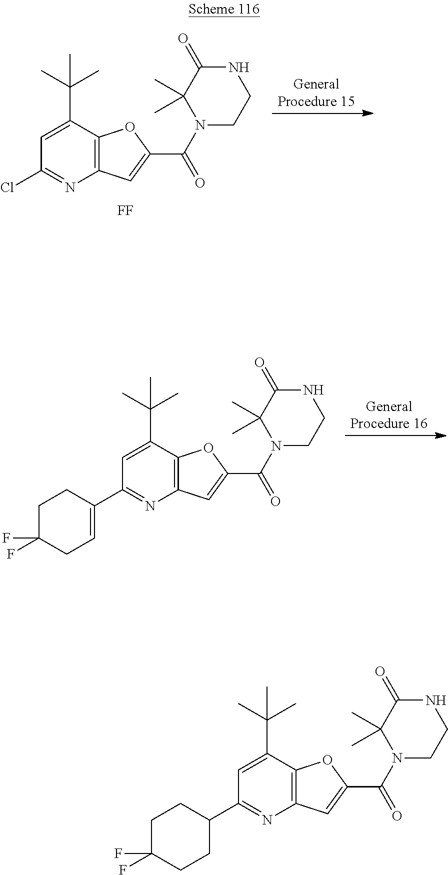

### Step I: 4-[7-tert-butyl-5-(4,4-difluorocyclohexen-1-yl)furo[3,2-b]pyridine-2-carbonyl]-3,3-dimethyl-piperazin-2-one

The product was prepared according to General Procedure 15 using 4-(7-tert-butyl-5-chloro-furo[3,2-b]pyridine-2-carbonyl)-3,3-dimethyl-piperazin-2-one (Intermediate FF) (88.21 mg, 0.2376 mmol), Pd2dba3 (6.8 mg, 0.0074 mmol), S-Phos (11.9 mg, 0.029 mmol), 2-(4,4-difluorocyclohexen-1-yl)-4,4,5,5-tetramethyl-1,3,2-dioxaborolane (116 mg, 0.48 mmol), 1,4-dioxane (1.24 mL) and K3PO4 (356 μL of 2 M, 0.71 mmol) affording the title compound. 4-[7-tert-butyl-5-(4,4-difluorocyclohexen-1-yl)furo[3,2-b]pyridine-2-carbonyl]-3,3-dimethyl-piperazin-2-one (19.3 mg, 0.043 mmol, 18%) ESI-MS m/z calc. 445.2177, found 446.70 (M+1)+; Retention time: 3.47 minutes using method A.

### Step II: 4-[7-tert-butyl-5-(4,4-difluorocyclohexyl)furo[3,2-b]pyridine-2-carbonyl]-3,3-dimethyl-piperazin-2-one

The product was prepared according to General Procedure 16 using -[7-tert-butyl-5-(4,4-difluorocyclohexen-1-yl)furo[3,2-b]pyridine-2-carbonyl]-3,3-dimethyl-piperazin-2-one (19.3 mg, 0.043 mmol), PtO₂ (1 mg, 0.0044 mmol) and methanol (1.5 mL) affording the title compound after mass-directed reverse phase HPLC. 4-[7-tert-butyl-5-(4,4-difluorocyclohexyl)furo[3,2-b]pyridine-2-carbonyl]-3,3-dimethyl-piperazin-2-one (12 mg, 0.02513 mmol, 57.11%). ¹H NMR (400 MHz, DMSO-d6) δ 8.16 (t, J=3.2 Hz. 1H), 7.45 (s, 1H), 7.17 (s, 1H), 3.70 (t, J=4.9 Hz, 2H), 3.38-3.32 (m, 2H), 3.04-2.88 (m, 1H), 2.16-1.97 (m, 3H), 1.97-1.76 (m, 6H), 1.65 (s, 6H), 1.42 (s, 9H). ESI-MS m/z calc. 447.2333, found 448.38 (M+1)⁺; Retention time: 3.02 minutes A. ESI-MS m/z calc. 479.17, found 481.39 (M+1)⁺; Retention time: 1.87 minutes using method C.

### Example 50: 4-[7-tert-butyl-5-(4,4-difluorocyclohexyl)furo[3,2-b]pyridine-2-carbonyl]-3,3-dimethyl-piperazin-2-one

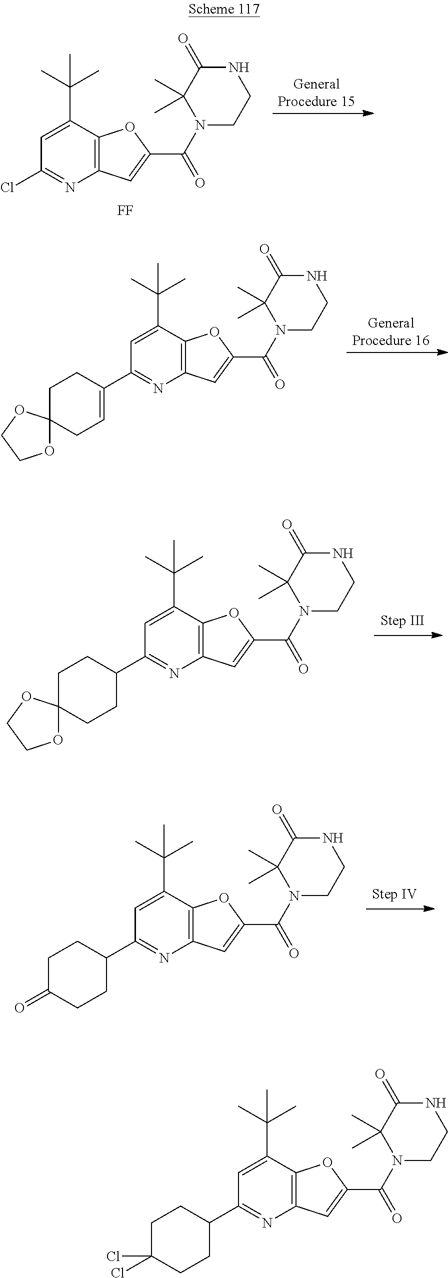

### Step I: 4-[7-tert-butyl-5-(1,4-dioxaspiro[4.5]dec-7-en-8-yl)furo[3,2-b]pyridine-2-carbonyl]-3,3-dimethyl-piperazin-2-one

The product was prepared according to General Procedure 15 using 4-(7-tert-butyl-5-chloro-furo[3,2-b]pyridine-2-carbonyl)-3,3-dimethyl-piperazin-2-one (200 mg, 0.5387 mmol) (Intermediate FF), Pd₂dba₃ (15.38 mg, 0.0168 mmol), dicyclohexyl-[2-(2,6-dimethoxyphenyl)phenyl]phosphane (26.91 mg, 0.06556 mmol), K₃PO₄ 2M in water (807.5 μL of 2 M, 1.62 mmol) and 2-(1,4-dioxaspiro[4.5]dec-7-en-8-yl)-4,4,5,5-tetramethyl-1,3,2-dioxaborolane (162.0 mg, 0.609 mmol) in 1,4-dioxane (2.8 mL) affording the title compound which was used in the subsequent step without further purification. 4-[7-tert-butyl-5-(1,4-dioxaspiro[4.5]dec-7-en-8-yl)furo[3,2-b]pyridine-2-carbonyl]-3,3-dimethyl-piperazin-2-one (251.9 mg, 100%). ESI-MS m/z calc. 467.24, found 468.08 (M+1)⁺; Retention time: 1.37 minutes using method C.

### Step II: 4-[7-tert-butyl-5-(1,4-dioxaspiro[4.5]decan-8-yl)furo[3,2-b]pyridine-2-carbonyl]-3,3-dimethyl-piperazin-2-one

The product was prepared according to General Procedure 16 using 4-[7-tert-butyl-5-(1,4-dioxaspiro[4.5]dec-7-en-8-yl)furo[3,2-b]pyridine-2-carbonyl]-3,3-dimethyl-piperazin-2-one (251.9 mg, 0.5388 mmol), dioxoplatinum (12.24 mg, 0.0539 mmol) and MeOH (19.38 mL) affording the title compound which was used in the subsequent step without further purification. 4-[7-tert-butyl-5-(1,4-dioxaspiro[4.5]decan-8-yl)furo[3,2-b]pyridine-2-carbonyl]-3,3-dimethyl-piperazin-2-one (253 mg, 0.5388 mmol, 100.0%). ESI-MS nm/z calc. 469.26, found 470.19 (M+1)⁺; Retention time: 2.51 minutes using method A.

### Step III: 4-[7-tert-butyl-5-(4-oxocyclohexyl)furo[3,2-b]pyridine-2-carbonyl]-3,3-dimethyl-piperazin-2-one

To a suspension of 4-[7-tert-butyl-5-(1,4-dioxaspiro[4.5]decan-8-yl)furo[3,2-b]pyridine-2-carbonyl]-3,3-dimethyl-piperazin-2-one (253 mg, 0.539 mmol) in dioxane (3.7 mL) and ethanol (913.3 μL) was added HCl (1.778 mL of 2 M, 3.56 mmol) and the mixture was stirred at 60° C. for 4 h. Water was added and the solutions cooled to room temperature. The mixture was extracted with EtOAc. The solvents were removed under reduced pressure and the residue was purified by silica chromatography affording the title compound. 4-[7-tert-butyl-5-(4-oxocyclohexyl)furo[3,2-b]pyridine-2-carbonyl]-3,3-dimethyl-piperazin-2-one (83 mg, 0.1951 mmol, 36.20%). ESI-MS m/z calc. 425.23, found 426.06 (M+1)⁺; Retention time: 1.20 minutes using method C.

### Step IV: 4-[7-tert-butyl-5-(4,4-dichlorocyclohexyl)furo[3,2-b]pyridine-2-carbonyl]-3,3-dimethyl-piperazin-2-one

To a mixture of 4A molecular sieves in MeOH (1.043 mL) was added hydrazine (Water (1)) (195.3 mg, 191.3 μL, 3.90 mmol) and 4-[7-tert-butyl-5-(4-oxocyclohexyl)furo[3,2-b]pyridine-2-carbonyl]-3,3-dimethyl-piperazin-2-one (83 mg, 0.195 mmol) and the mixture was stirred at room temp for 2 h. The suspension was then filtered on celite, washed with MeOH and the solvents were removed under reduced pressure.

In a seperated flask, Et₃N (69.10 mg, 95.18 μL, 0.683 mmol) was added dropwise to a suspension of CuCl₂ (183.7 mg, 1.366 mmol) in MeOH (622.5 μL) at 0° C. The mixture was stirred cold for 30 minutes and for 2 h at room temperature. The mixture was brought back to 0° C., the previously formed hydrazone was added and the solution was stirred for 2 h. Water was added and the aq. phase was extracted twice with EtOAc. The combined organic phase was washed with 1N HCl, saturated aqueous NaHCO₃ and brine. The organic phase was then dried over MgSO₄, filtered and the filtrate evaporated under reduced pressure by mass-directed reverse phase HPLC affording the title compound, 4-[7-tert-butyl-5-(4,4-dichlorocyclohexyl)furo[3,2-b]pyridine-2-carbonyl]-3,3-dimethyl-piperazin-2-one (6.3 mg, 6.2%). ¹H NMR (400 MHz, DMSO-d6) δ 8.16 (t, J=3.3 Hz, 1H), 7.48 (s, 1H), 7.15 (s, 1H), 3.74-3.64 (m, 2H), 3.35-3.31 (m, 2H), 2.55 (d, J=13.5 Hz, 2H), 2.44-2.33 (m, 2H), 2.10-1.93 (m, 3H), 1.88 (dd, J=9.7, 5.2 Hz, 2H), 1.65 (s, 6H), 1.42 (s, 8H). ESI-MS m/z calc. 479.17, found 481.39 (M+1)⁺; Retention time: 1.87 minutes using method C.

### Example 51: 6-[4-[7-tert-butyl-5-(4,4-dimethylcyclohexyl)furo[3,2-b]pyridine-2-carbonyl]-3,3-dimethyl-piperazin-1-yl]pyridine-2-carboxylic acid

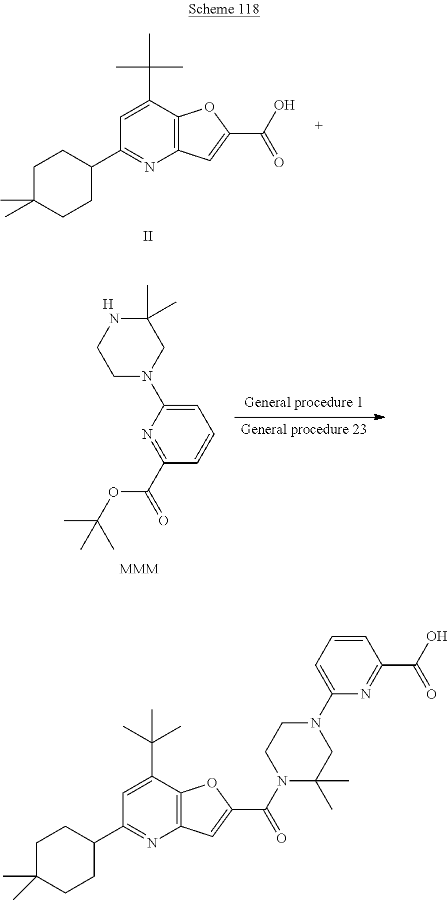

The product was prepared according to General Procedure 1 using Intermediates II and MMM in NMP (7.203 mL) using DIPEA (472.3 mg, 636.5 μL, 3.654 mmol) and HATU (715.6 mg, 1.88 mmol) to afford the tert-butyl intermediate (770 mg, 1.28 mmol). The ester intermediate was then hydrolyzed using General Procedure 23, in 1,4-dioxane (7.7 mL) with HCl (1.6 mL of 4 M. 6.4 mmol) in dioxane to generate 6-[4-[7-tert-butyl-5-(4,4-dimethylcyclohexyl)furo[3,2-b]pyridine-2-carbonyl]-3,3-dimethyl-piperazin-1-yl]pyridine-2-carboxylic acid. ESI-MS m/z calc. 546.3206, found 547.02 (M+1)+.

### Example 52: ethyl 2-[4-[7-tert-butyl-5-(4,4-dimethylcyclohexyl)furo[3,2-b]pyridine-2-carbonyl]-3,3-dimethyl-piperazin-1-yl]oxazole-4-carboxylate

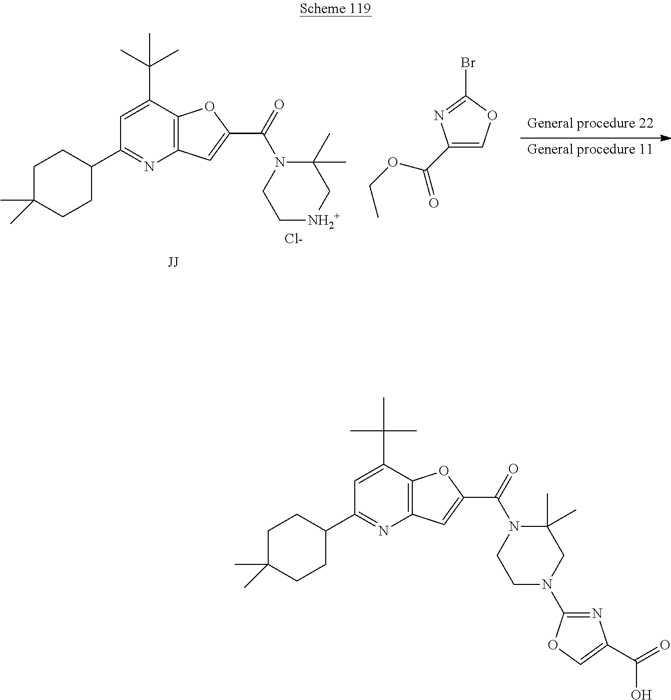

The ethyl ester was prepared according to General Procedure 22, Method A, using Intermediate JJ, ethyl 2-bromooxazole-4-carboxylate (99.98 mg, 0.454 mmol) and Cs2CO3 (422.9 mg, 1.30 mmol) in NMP (2.2 mL) at 100° C. for 4 h. ESI-MS m/z calc. 564.3312, found 565.66 (M+1)+; Retention time: 1.53 minutes. 1H NMR (400 MHz, Methanol-d4) δ 8.02 (s, 1H), 7.33 (s, 1H), 7.21 (s, 1H), 4.29 (q, J=7.1 Hz, 2H), 4.12-4.02 (m, 3H), 3.79-3.72 (m, 4H), 2.78-2.66 (m, 1H), 1.91-1.72 (m, 3H), 1.76-1.67 (m, 2H), 1.61 (s, 6H), 1.57-1.47 (m 3H), 1.51 (s, 9H), 1.45-1.34 (m, 2H), 1.32 (t, J=7.1 Hz, 3H), 1.02 (s, 4H), 0.96 (s, 3H). The product was then prepared from the ethyl ester intermediate using General Procedure 11 with a 2.5N aqueous solution of NaOH (318.7 μL of 2.5 M, 0.7968 mmol), in a 1:1 mixture of THF (664.0 μL)/Ethanol (664.0 μL) at rt for 1 h. ESI-MS m/z calc. 536.29987, found 537.96 (M+1)+; 1H NMR (400 MHz, DMSO-d6) (12.72 (s, 1H, broad), 8.23 (s, 1H), 7.43 (s, 1H), 7.16 (s, 1H), 3.97 (t, J=5.7 Hz, 2H), 3.69-3.60 (m, 4H), 2.77-2.64 (m, 1H), 1.87-1.64 (m, 4H), 1.53 (s, 6H), 1.51-1.43 (m, 11H), 1.40-1.27 (m, 2H), 0.98 (s, 3H), 0.95 (s, 3H).

### Example 53: 2-[4-[7-tert-butyl-5-(4,4-dimethylcyclohexyl)furo[3,2-b]pyridine-2-carbonyl]-3,3-dimethyl-piperazin-1-yl]pyrimidine-4-carboxylate

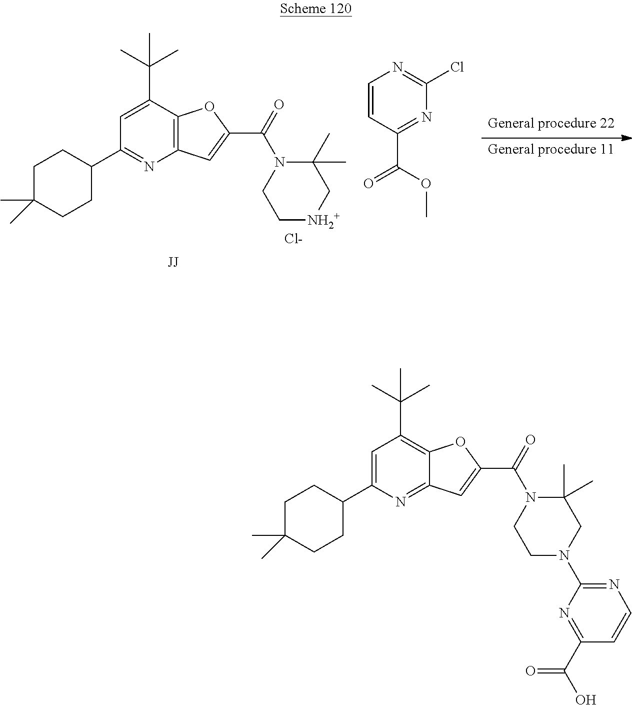

The ethyl ester was prepared according to General Procedure 22, Method A, using Intermediate JJ, methyl 2-chloropyrimidine-4-carboxylate (56.02 mg, 0.325 mmol) and DIPEA (168.4 mg, 227.0 μL, 1.30 mmol), in dioxane (1.500 mL) at 120° C. for 1 h in microwave. The product was then prepared from the ethyl ester intermediate using General Procedure 11 without purification of the ester, adding NaOH (486.9 μL of 2 M, 0.974 mmol) directly to the reaction mixture, at 80° C. for 2 h to afford 2-[4-[7-tert-butyl-5-(4,4-dimethylcyclohexyl)furo[3,2-b]pyridine-2-carbonyl]-3,3-dimethyl-piperazin-1-yl]pyrimidine-4-carboxylic acid 1H NMR (400 MHz, DMSO-d6) δ 8.58 (d, J=4.8 Hz, 1H), 7.40 (s, 1H), 7.13 (s, 1H), 7.10 (d, J=4.8 Hz, 1H), 4.11-3.93 (m, 4H), 3.73 (t, J=5.7 Hz, 2H), 2.75-2.65 (m, 1H), 1.81-1.58 (m, 4H), 1.50 (s, 6H), 1.43 (s, 9H), 1.42-1.41 (m, 2H), 1.30 (td, J=13.1, 4.2 Hz, 2H), 0.94 (s, 3H), 0.92 (s, 4H). ESI-MS m/z calc. 547.31586, found 550.43 (M+1)+;

### Example 54: 2-[4-[7-tert-butyl-5-(4,4-dimethylcyclohexyl)furo[3,2-b]pyridine-2-carbonyl]-3,3-dimethyl-piperazin-1-yl]pyrimidine-4-carboxylate

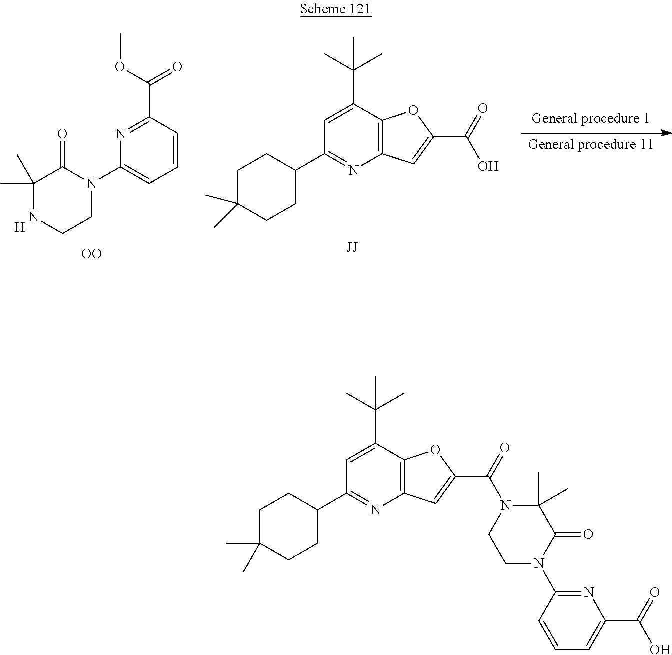

The ethyl ester was prepared according to General Procedure 1 using Intermediates JJ and OO, HATU (68 mg, 0.179 mmol), and DIPEA (75 μL, 0.431 mmol) in DMF (1.5 mL) for three days. ESI-MS m/z calc. 574.3155, found 575.45 (M+1)+: The methyl ester was then hydrolyzed according to General Procedure 11 using NaOH (500 μL of 1 M, 0.50 mmol) in MeOH (10 mL) for 2 h at r.t. to afford 6-[4-[7-tert-butyl-5-(4,4-dimethylcyclohexyl)furo[3,2-b]pyridine-2-carbonyl]-3,3-dimethyl-2-oxo-piperazin-1-yl]pyridine-2-carboxylic acid (7.5 mg, 11%) 1H NMR (400 MHz, DMSO-d6) δ 13.30 (s, 1H), 8.13-8.07 (m, 1H), 8.07-8.00 (m, 1H), 7.90 (dd, J=7.3, 1.0 Hz; 1H), 7.55 (s, 1H), 7.19 (s, 1H), 4.33-4.23 (m, 2H), 4.10-4.01 (m, 2H), 2.78-2.64 (m, 1H), 1.85 (s, 6H), 1.82-1.65 (m, 4H), 1.53-1.42 (m, 11H), 1.40-1.28 (m, 2H), 0.97 (d, J=9.9 Hz, 6H). ESI-MS m/z calc. 560.29987, found 561.28 (M+1)+.

### Example 55: 5-[4-[7-tert-butyl-5-(4-chlorophenyl)furo[3,2-b]pyridine-2-carbonyl]-3,3-dimethyl-piperazin-1-yl]pyridine-3-carboxylic acid

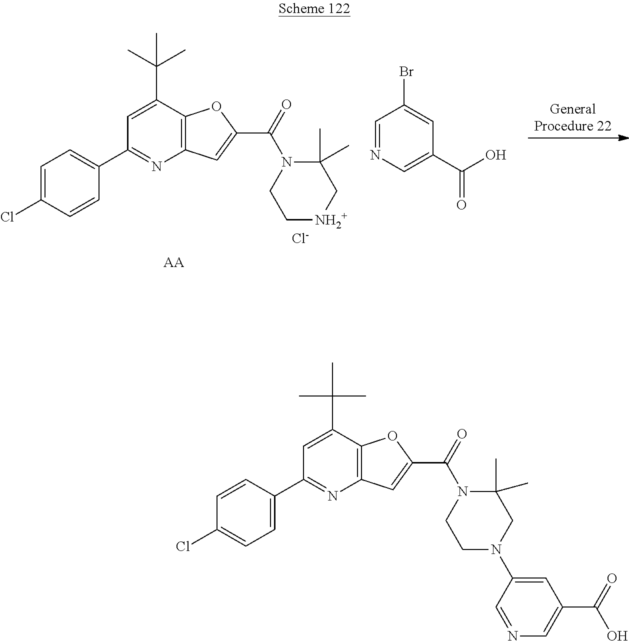 [SMILES: CC(C)(C)c1cc(-c2ccc(Cl)cc2)nc2cc(C(=O)N3CC[NH2+]CC3(C)C)oc12 ; O=C(O)c1cncc(Br)c1 ; "GeneralProcedure 22" ; [Cl-] ; "AA" ; CC(C)(C)c1cc(-c2ccc(Cl)cc2)nc2cc(C(=O)N3CCN(c4cncc(C(=O)O)c4)CC3(C)C)oc12]

The product was prepared according to General Procedure 22, Method B, using intermediate AA (100 mg, 0.2163 mmol). NaOtBu (94 mg, 0.978 mmol), RUPHOS (5 mg, 0.0107 mmol), Ruphos (IV) (CAS:1599466-85-9) (9 mg, 0.0106 mmol) and 5-bromopyridine-3-carboxylic acid (66 mg, 0.327 mmol) in dioxane (2 mL) at 90° C. for 18 h to afford 5-[4-[7-tert-butyl-5-(4-chlorophenyl)furo[3,2-b]pyridine-2-carbonyl]-3,3-dimethyl-piperazin-1-yl]pyridine-3-carboxylic acid (9.1 mg, 0.0154 mmol, 7%). 1H NMR (400 MHz, DMSO-d6) δ 8.39 (d, J=1.5 Hz, 1H), 8.36 (d, J=3.0 Hz, 1H), 8.21-8.11 (m, 2H), 7.79 (s, 1H), 7.60-7.54 (m, 3H), 7.52 (dd, J=3.1, 1.7 Hz, 1H), 4.09 (t, J=5.2 Hz, 2H), 3.69 (s, 2H), 3.56 (t, J=5.4 Hz, 2H), 1.59-1.48 (m, 15H). ESI-MS m/z calc. 546.20337, found 547.53 (M+1)+.

### Example 56: 5-[4-[7-tert-butyl-5-(4-chlorophenyl)furo[3,2-b]pyridine-2-carbonyl]-3,3-dimethyl-piperazin-1-yl]pyridine-3-carboxylic acid

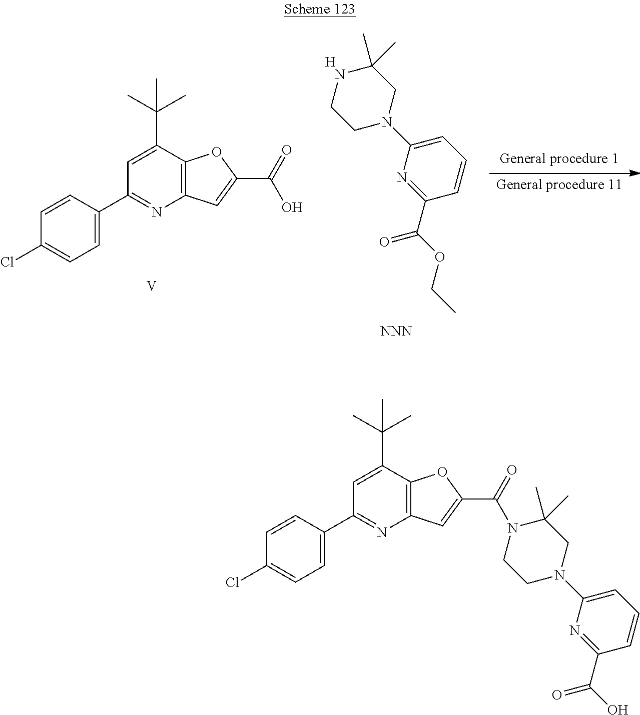

The ethyl ester was prepared according to General Procedure 1 using Intermediates AA and NNN, DIPEA (1.079 g, 1.45 mL, 8.35 mmol) and HATU (635.0 mg, 1.67 mmol) in DMF (4.4 mL) for 1 h at r.t. The ethyl ester was then hydrolyzed according to General Procedure 11 using LiOH (4 eq, 2.46 mmol, 1.2 mL, 2M) in dioxane (4.4 mL) for 4 h at r.t. to afford the title product (347 mg, 36%). 1H NMR (400 MHz, dmso) δ 12.57 (s, 1H), 8.19-8.07 (m, 2H), 7.75 (s, 1H), 7.69 (dd, J=8.6, 7.2 Hz, 1H), 7.62-7.51 (m, 3H), 7.27 (d, J=7.2 Hz, 1H), 6.84 (d, J=8.6 Hz, 1H), 4.12-4.03 (m, 2H), 3.98 (s, 2H), 3.66-3.60 (m, 2H), 1.61-1.40 (m, 15H). ESI-MS m/z calc. 546.20337, found 547.48 (M+1)+.

### Example 57: [7-tert-butyl-5-(4-chloro-3-fluoro-phenyl)furo[3,2-b]pyridin-2-yl]-[4-[(3R)-1,1-dioxo-1,2-thiazolidine-3-carbonyl]-2,2-dimethyl-piperazin-1-yl]methanone and [7-tert-butyl-5-(4-chloro-3-fluoro-phenyl)furo[3,2-b]pyridin-2-yl]-[4-[(3S)-1,1-dioxo-1,2-thiazolidine-3-carbonyl]-2,2-dimethyl-piperazin-1-yl]methanone

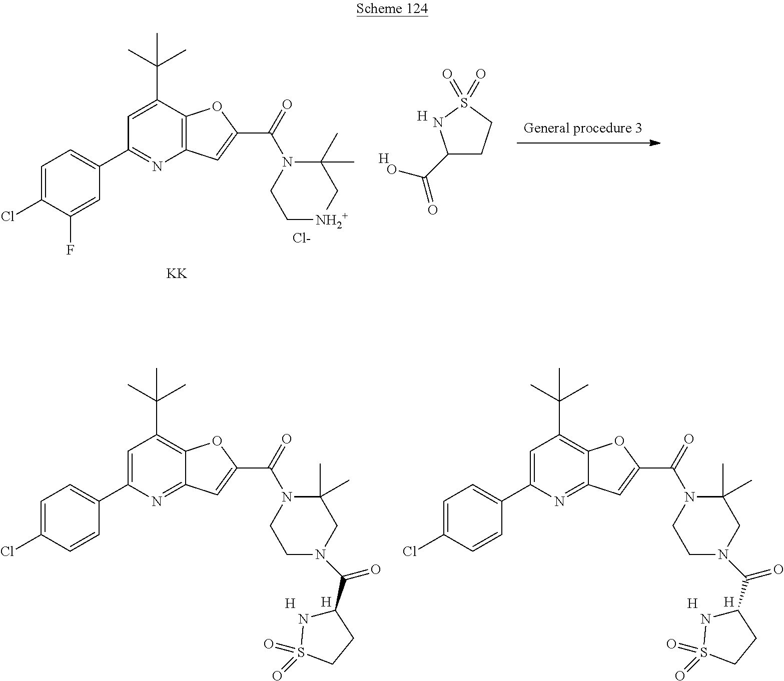

The ethyl ester was prepared according to General Procedure 3 using Intermediates KK. 1,1-dioxo-1,2-thiazolidine-3-carboxylic acid (50 mg, 0.302 mmol), DIPEA (0.26 mL, 1.51 mmol) and HATU (126 mg, 0.333 mmol) in DMF (1.5 mL) for 2.5 h at RT. The products were then subjected to SFC chiral separation to afford [7-tert-butyl-5-(4-chloro-3-fluoro-phenyl)furo[3,2-b]pyridin-2-yl]-[4-[(3R)-1,1-dioxo-1,2-thiazolidine-3-carbonyl]-2,2-dimethyl-piperazin-1-yl]methanone ESI-MS m/z calc. 590.1766, found 591.47 (M+1)+; Retention time: 3.73 minutes and [7-tert-butyl-5-(4-chloro-3-fluoro-phenyl)furo[3,2-b]pyridin-2-yl]-[4-[(3S)-1,1-dioxo-1,2-thiazolidine-3-carbonyl]-2,2-dimethyl-piperazin-1-yl]methanone ESI-MS m/z calc. 590.1766, found 591.47 (M+1)+; Retention time: 3.73 minutes.

### Example 58: 4-[7-tert-butyl-5-(4-chloro-3-fluoro-phenyl)furo[3,2-b]pyridine-2-carbonyl]-3,3-dimethyl-1-methylsulfonyl-piperazin-2-one

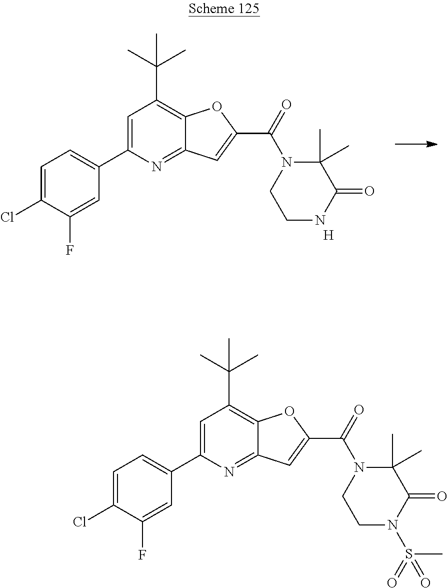

A solution of 4-[7-tert-butyl-5-(4-chloro-3-fluoro-phenyl)furo[3,2-b]pyridine-2-carbonyl]-3,3-dimethyl-piperazin-2-one (88 mg, 0.19 mmol) in THF (1 mL) was cooled to −78° C. and treated with LiHMDS (1M, 211 μL, 0.211 mmol). The mixture was stirred for 20 min, then a solution of methanesulfonyl chloride (18 μL, 0.23 mmol) in THF (88 μL) was added dropwise. The mixture was stirred for 2 h then allowed to warm to rt for 2 h. The reaction mixture was quenched with water, and the resulting mixture was extracted three times with EtOAc. The combined organic layers were dried over sodium sulfate, filtered, and the solvent was removed under reduced pressure. The crude product was purified by mass-directed reverse-phase HPLC affording the title compound, after lyophilization, 4-[7-tert-butyl-5-(4-chloro-3-fluoro-phenyl)furo[3,2-b]pyridine-2-carbonyl]-3,3-dimethyl-1-methylsulfonyl-piperazin-2-one (39 mg, 0.072 mmol, 38%). 1H NMR (400 MHz, DMSO-d6) δ 8.14 (dd, J=11.0, 2.1 Hz, 1H), 8.01 (dd, J=8.5, 2.0 Hz, 1H), 7.82 (s, 1H), 7.69 (t, J=8.2 Hz, 1H), 7.61 (s, 1H), 3.95 (s, 4H), 3.44 (s, 3H), 1.75 (s, 6H), 1.51 (s, 9H). ESI-MS m/z calc. 535.1344, found 536.63 (M+1)+; Retention time: 4.45 minutes using method A.

### Example 59: (S)-2-amino-5-(4-(7-(tert-butyl)-5-(4,4-dimethylcyclohexyl)furo[3,2-b]pyridine-2-carbonyl)-3,3-dimethylpiperazin-1-yl)-5-oxopentanoic acid formic acid salt

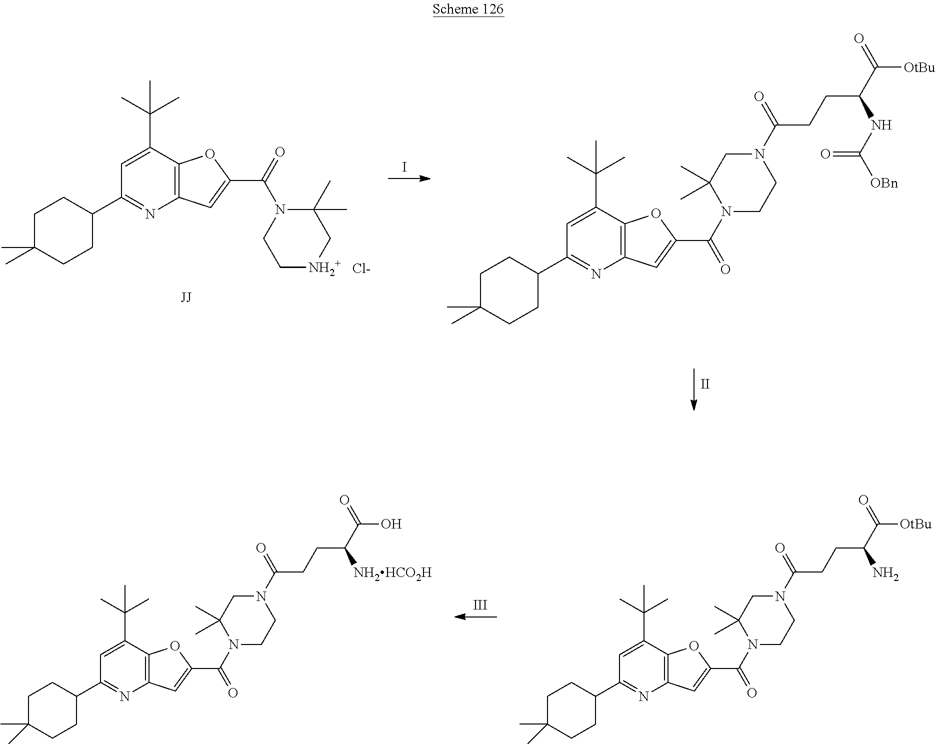

### Step I: tert-butyl (2S)-2-(benzyloxycarbonylamino)-5-[4-[7-tert-butyl-5-(4,4-dimethylcyclohexyl)furo[3,2-b]pyridine-2-carbonyl]-3,3-dimethyl-piperazin-1-yl]-5-oxo-pentanoate

According to General Procedure 3 using Intermediate JJ (90 mg, 0.1948 mmol), HATU (96 mg, 0.2525 mmol), DMF (1 mL), Hünig's base (140 μL, 0.8038 mmol) and (4S)-4-(benzyloxycarbonylamino)-5-tert-butoxy-5-oxo-pentanoic acid (85 mg, 0.2520 mmol) afforded the title compound tert-butyl (2S)-2-(benzyloxycarbonylamino)-5-[4-[7-tert-butyl-5-(4,4-dimethylcyclohexyl)furo[32-b]pyridine-2-carbonyl]-3,3-dimethyl-piperazin-1-yl]-5-oxo-pentanoate (105 mg, 72%). ESI-MS m/z calc. 744.4462, found 745.24 (M+1)⁺; Retention time: 2.37 minutes using method C.

### Step II: tert-butyl (S)-2-amino-5-(4-(7-(tert-butyl)-5-(4,4-dimethylcyclohexyl)furo[3,2-b]pyridine-2-carbonyl)-3,3-dimethylpiperazin-1-yl)-5-oxopentanoate

To a solution of tert-butyl (2S)-2-(benzyloxycarbonylamino)-5-[4-[7-tert-butyl-5-(4,4-dimethylcyclohexyl)furo[3,2-b]pyridine-2-carbonyl]3,3-dimethyl-piperazin-1-yl]-5-oxo-pentanoate (105 mg, 0.1409 mmol) in MeOH (5 mL) was added 10% Pd/C (20 mg, 0.019 mmol). The nitrogen atmosphere was evacuated under reduced pressure and re-filled with hydrogen 3 times, and the reaction mixture was stirred under a hydrogen atmosphere (normal pressure) at rt for 2 h. The reaction mixture was filtered and the volatiles were removed under reduced pressure to afford the title compound tert-butyl (2S)-2-amino-5-[4-[7-tert-butyl-5-(4,4-dimethylcyclohexyl)furo[3,2-b]pyridine-2-carbonyl]-3,3-dimethyl-piperazin-1-yl]-5-oxo-pentanoate (85 mg, 0.14 mmol, 99%). ESI-MS m/z calc. 610.4094, found 611.63 (M+1)⁺; Retention time: 1.55 minutes using method C.

### Step III: (S)-2-amino-5-(4-(7-(tert-butyl)-5-(4,4-dimethylcyclohexyl)furo[3,2-b]pyridine-2-carbonyl)-3,3-dimethylpiperazin-1-yl)-5-oxopentanoic acid formic acid salt

The solution of tert-butyl (2S)-2-amino-5-[4-[7-tert-butyl-5-(4,4-dimethylcyclohexyl)furo[3,2-b]pyridine-2-carbonyl]-3,3-dimethyl-piperazin-1-yl]-5-oxo-pentanoate (85 mg, 0.1392 mmol) in formic acid (1 mL) was heated at 60° C. for 5 h. The volatiles were removed under reduced pressure and the residue was freeze-dried to obtain (2S)-2-amino-5-[4-[7-tert-butyl-5-(4,4-dimethylcyclohexyl)furo[3,2-b]pyridine-2-carbonyl]-3,3-dimethyl-piperazin-1-yl]-5-oxo-pentanoic acid formic acid salt (78 mg). 1H NMR (400 MHz, DMSO-d6) δ 6.50 (d, J=0.6 Hz, 1H), 6.40 (d, J=1.0 Hz, 1H), 3.37-3.07 (m, 2H), 3.02-2.72 (m, 5H), 2.02-1.71 (m, 3H), 1.45-1.25 (m, 2H), 1.12-0.88 (m, 4H), 0.82-0.70 (m, 17H), 0.59 (m 2H), 0.22 (s, 3H), 0.16 (s, 3H). ESI-MS m/z calc. 554.3468, found 555.35 (M+1)+; Retention time: 2.77 minutes using Method A.

### Example 60: 2-(4-(7-(tert-butyl)-5-(4-chlorophenyl)furo[3,2-b]pyridine-2-carbonyl)-3,3-dimethyl-2-oxopiperazin-1-yl)pyrimidine-5-carboxylic acid

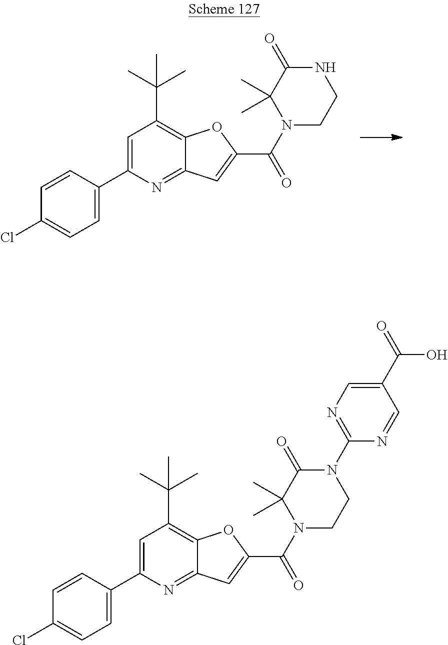 [SMILES: CC(C)(C)c1cc(-c2ccc(Cl)cc2)nc2cc(C(=O)N3CCNC(=O)C3(C)C)oc12 ; CC(C)(C)c1cc(-c2ccc(Cl)cc2)nc2cc(C(=O)N3CCN(c4ncc(C(=O)O)cn4)C(=O)C3(C)C)oc12]

A degassed mixture of methyl 2-chloropyrimidine-5-carboxylate (60 mg, 0.35 mmol), 4-[7-tert-butyl-5-(4-chlorophenyl)furo[3,2-b]pyridine-2-carbonyl]-3,3-dimethyl-piperazin-2-one (150 mg, 0.341 mmol). Pd(OAc)₂ (11 mg, 0.049 mmol), Xantphos (31 mg, 0.054 mmol) and Cs₂CO₃ (158 mg, 0.48 mmol) in dioxane (1.5 mL) was heated at 100° C. for 48 h. The mixture was allowed to cool to rt then diluted with EtOAc, water and 1N HCl. The layers were separated and the aqueous layer was back-extracted twice with EtOAc. The combined organic extracts were washed with brine, dried over MgSO4, filtered and concentrated under reduced pressure. The crude product was purified by mass-directed reverse-phase HPLC affording the title compound, after lyophilization2-(4-(7-(tert-butyl)-5-(4-chlorophenyl)furo[3,2-b]pyridine-2-carbonyl)-3,3-dimethyl-2-oxopiperazin-1-yl)pyrimidine-5-carboxylic acid (4.7 mg, 0.007242 mmol, 2.124%). 1H NMR (400 MHz, DMSO-d6) δ 9.14 (s, 2H), 8.23-8.02 (m, 2H), 7.79 (s, 1H), 7.65 (s, 1H), 7.60-7.47 (m, 2H), 4.31-4.20 (m, 2H), 4.14-3.98 (m, 2H), 1.83 (s, 5H), 1.52 (s, 9H). ESI-MS m/z calc. 561.1779, found 563.28 (M+1)⁺; Retention time: 3.97 minutes using method A.

### Example 61: Preparation of 4-[7-tert-butyl-5-(4-chloro-3-fluoro-phenyl)furo[3,2-b]pyridine-2-carbonyl]-3,3-dimethyl-1-(2-methylpyrazol-3-yl)sulfonyl-piperazin-2-one

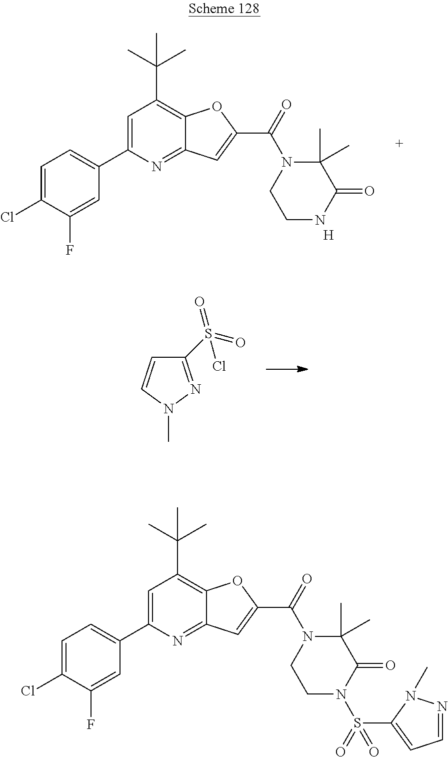

To A solution of 4-[7-tert-butyl-5-(4-chloro-3-fluoro-phenyl)furo[3,2-b]pyridine-2-carbonyl]-3,3-dimethyl-piperazin-2-one (88 mg, 0.1922 mmol) in THF (1.1 mL) was cooled to −78° C. and treated with LiHMDS (211 μL of 1 M, 0.211 mmol). The mixture was stirred for 20 min, then a solution of 1-methylpyrazole-3-sulfonyl chloride (41.65 mg, 0.2306 mmol) in THF (88 μL) was added dropwised. The mixture was stirred for 2 h at −78° C. then warmed to rt and was stirred for an extra 4 hr. Water was added to the solution and the aqueous phase was extracted 3 times with EtOAc. The combined organic phase was dried over Na₂SO₄ and filtered. The filtrate was evaporated under reduced pressure and the residue was purified using reverse phase preperative HPLC affording the title compound after lyophilisation. 4-[7-tert-butyl-5-(4-chloro-3-fluoro-phenyl)furo[3,2-b]pyridine-2-carbonyl]-3,3-dimethyl-1-(2-methylpyrazol-3-yl)sulfonyl-piperazin-2-one (34 mg, 29% yield). ESI-MS m/z calc. 601.1562, found 602.01 (M+1)+; Retention time: 4.34 minutes using method A.

### Example 62: Preparation of 4-[7-tert-butyl-5-(4,4-dimethylcyclohexyl)furo[3,2-b]pyridine-2-carbonyl]-3,3-dimethyl-1-(5-methyl-1H-pyrazol-3-yl)piperazin-2-one

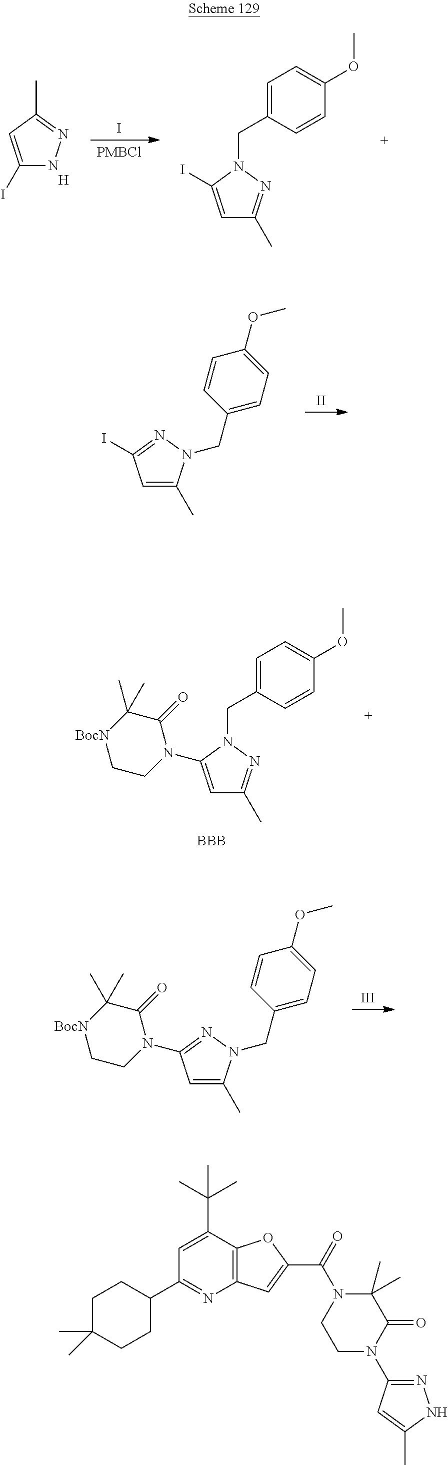

### Step I: 5-iodo-1-[(4-methoxyphenyl)methyl]-3-methyl-pyrazole and 3-iodo-1-[(4-methoxyphenyl)methyl]-5-methyl-pyrazole

1-(chloromethyl)-4-methoxy-benzene (1.60 mL, 11.80 mmol) was added to a stirred mixture of 5-iodo-3-methyl-1H-pyrazole hydrochloride salt (2.0 g, 8.18 mmol) and Cs₂CO₃ (8.02 g, 24.6 mmol) in DMF (30 mL) and the mixture was stirred at 60° C. for 1.5 h. The reaction was cooled toRT, diluted with H₂O (100 mL) and extracted with EtOAc (3×50 mL). The combined organic extracts were washed with saturated NH₄Cl (25 mL), brine (25 mL), dried over MgSO₄, filtered and concentrated. The residue was purified by flash chromatography on silica gel eluting with EtOAc in hexanes, 0% (1CV), 0-20% (10CV), 20% (2CV). Obtained a colorless solid as a (2.62 g, 98%). By NMR the material is a 2:1 mixture of isomers. For the major isomer: ¹H NMR (400 MHz, cdcl3) δ 7.10-7.01 (m, 2H), 6.89-6.79 (m, 2H), 6.19 (d, J=0.6 Hz, 1H), 5.21 (s, 2H), 3.78 (s, 3H), 2.17 (d, J=0.6 Hz, 3H). ESI-MS m/z calc. 328.00726, found 328.93 (M+1)+; Retention time: 0.8 minutes using Method J.

### Step II. tert-butyl 4-(1-(4-methoxybenzyl)-3-methyl-1H-pyrazol-5-yl)-2,2-dimethyl-3-oxopiperazine-1-carboxylate and tert-butyl 4-(1-(4-methoxybenzyl)-5-methyl-1H-pyrazol-3-yl)-2,2-dimethyl-3-oxopiperazine-1-carboxylate

A RBF was charged with tert-butyl 2,2-dimethyl-3-oxo-piperazine-1-carboxylate (2.00 g, 8.76 mmol), the mixture of regioisomers prepared above (2.97 g, 9.051 mmol), K₃PO₄ (3.77 g, 17.76 mmol) in DMF (40 mL). The mixture was degassed and N,N-dimethylethylenediamine (580 μL, 5.45 mmol) and CuI (848 mg, 4.45 mmol) were added and the reaction heated at 120° C. for 5.5 h. The reaction was diluted with H₂O (50 mL), saturated NH₄Cl (50 mL) and EtOAc (100 mL). The layers were separated and the aqueous layer extracted with EtOAc (2×100 mL). Combined organic extracts were washed with saturated NH₄Cl (2×50 mL), brine (50 mL), dried over MgSO₄, filtered and concentrated to give 4.57 g. The crude material was dissolved in DCM (20 mL), HCl in dioxane (11.0 mL 4 M, 43.8 mmol) and the mixture was stirred for 6 h. The reaction was concentrated in vacuo and coevaporated from DCM/Et₂O three times. The residue was triturated with heptane to afford the title compounds. 1-[2-[(4-methoxyphenyl)methyl]-5-methyl-pyrazol-3-yl]-3,3-dimethyl-piperazin-2-one (Hydrochloride salt) (3.44 g, 71%). ESI-MS m/z calc. 328.1899, found 330.42 (M+1)+; Retention time: 0.37 minutes using method J.

### Step III

### 4-[7-tert-butyl-5-(4,4-dimethylcyclohexyl)furo[3,2-b]pyridine-2-carbonyl]-3,3-dimethyl-1-(5-methyl-1H-pyrazol-3-yl)piperazin-2-one

According to General Procedure 3, a solution of the material prepared in the previous step 7-tert-butyl-5-(4,4-dimethylcyclohexyl)furo[3,2-b]pyridine-2-carboxylic acid (100.3 mg, 0.305 mmol) and the material prepared in the previous step (100 mg, 0.305 mmol) in DMF (1.5 mL) was reacted with DIPEA (196.7 mg, 265.1 μL, 1.52 mmol) and HATU (138.9 mg, 0.365 mmol) for 1 h at rt. The reaction mixture was then diluted with water (1.5 mL) and filtered to give a beige solid which was treated with TFA (1.5 mL) and heated in the microwave at 130° C. for 25 min. The reaction mixture was evaporated in vacuo, dissolved in DMSO and purified by prepartive HPLC to give 4-[7-tert-butyl-5-(4,4-dimethylcyclohexyl)furo[3,2-b]pyridine-2-carbonyl]-3,3-dimethyl-1-(5-methyl-1H-pyrazol-3-yl)piperazin-2-one (1.8 mg, 1%). ¹H NMR (400 MHz, DMSO-d6) δ 12.24 (d, J=2.3 Hz, 1H), 7.49 (s, 1H), 7.15 (s, 1H), 6.44 (t, J=1.5 Hz, 1H), 4.23-3.87 (m, 4H), 2.80-2.62 (m, 1H), 2.19 (s, 3H), 1.89-1.71 (m, 8H), 1.70-1.58 (m, 2H), 1.52-1.45 (m, 2H), 1.43 (s, 9H), 1.35-1.17 (m, 2H), 0.95 (s, 3H), 0.92 (s, 3H). ESI-MS m/z calc. 519.3209, found 520.59 (M+1)+; Retention time: 2.16 minutes using Method C.

### Example 63: Preparation of 1-[5-(4-chloro-3-fluoro-phenyl)-2-[4-(3-hydroxy-3-methyl-cyclobutanecarbonyl)-2,2-dimethyl-piperazine-1-carbonyl]furo[3,2-b]pyridin-7-yl]-3,3-difluoro-cyclobutanecarbonitrile

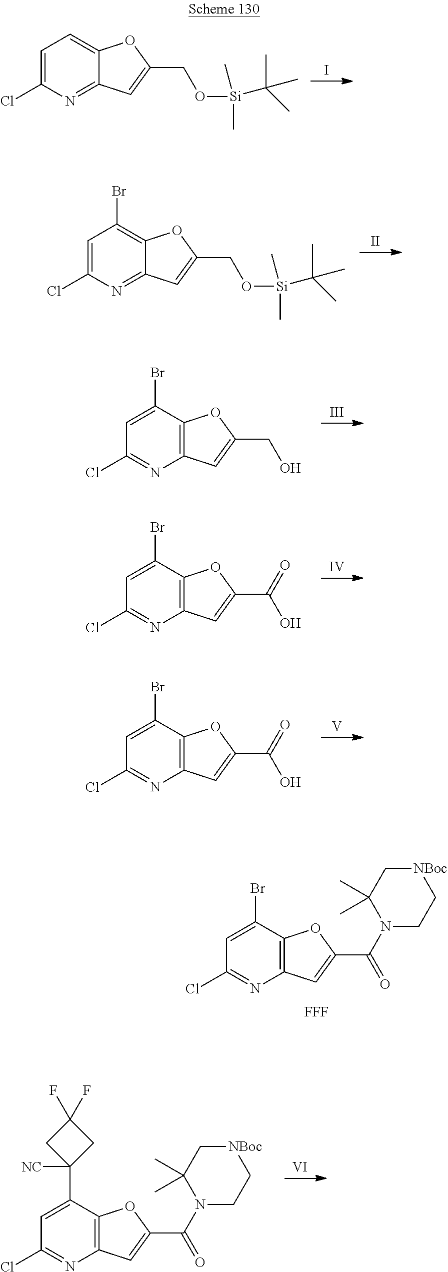

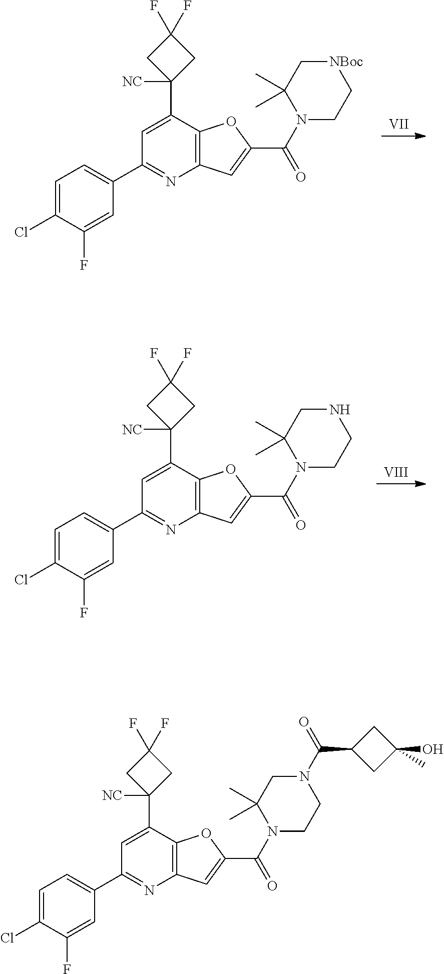

### Step I: (7-bromo-5-chloro-furo[3,2-b]pyridin-2-yl)methoxy-tert-butyl-dimethyl-silane

A solution of 5-chloro-furo[3,2-b]pyridin-2-yl)methoxy-tert-butyl-dimethyl-silane (500 mg, 1.68 mmol) in THF (20 mL) was cooled to −78° C. and nBuLi (915.9 μL, 2.2 M, 2.02 mmol) is added dropwise over 5 minutes. The mixture was stirred for 60 minutes at −78 C and 1,2-dibromo-1,1,2,2-tetrachloro-ethane (683.5 mg, 2.1 mmol) was added. The mixture was stirred at −78° C. for 60 minutes then warmed up to 0° C. over 60 minutes. The reaction mixture was absorbed on silica gel then purified by flash chromatography using 0-50% DCM/Hexanes as eluent to generate the title compound.

### Step II: (7-bromo-5-chloro-furo[3,2-b]pyridin-2-yl)methanol

A solution of (7-bromo-5-chloro-furo[3,2-b]pyridin-2-yl)methoxy-tert-butyl-dimethyl-silane (20.2 g, 53.7 mmol) in 1,4-dioxane (202.2 mL) was stirred at RT. HCl (40.25 mL 4 M, 161.0 mmol) in dioxane was added dropwise then stirred at RT under N2 (g) overnight. The suspension was cooled to 5-10° C. then filtered. The solids were dried under vacuum to generate the title compound. The crude material was used as is.

### Step III: 7-bromo-5-chloro-furo[3,2-b]pyridine-2-carboxylic acid

To (7-bromo-5-chloro-furo[3,2-b]pyridin-2-yl)methanol (627 mg, 2.39 mmol) and 4-methyl-4-oxido-morpholin-4-ium hydrate (3.66 g. 23.9 mmol) in MeCN (8.76 mL) was added TPAP (84.0 mg, 0.239 mmol). After stirring for 30 min, the reaction mixture was treated with iPrOH (1.447 g, 1.843 mL, 24.08 mmol). The mixture was diluted with H₂O and EtOAc, the phases were separated and the solvent removed in vacuo to give 7-bromo-5-chloro-furo[3,2-b]pyridine-2-carboxylic acid (660.5 mg, 2.389 mmol, 100.0%).

### Step IV: tert-butyl 4-(7-bromo-5-chloro-furo[3,2-b]pyridine-2-carbonyl)-3,3-dimethyl-piperazine-1-carboxylate

To a solution of 7-bromo-5-chloro-furo[3,2-b]pyridine-2-carboxylic acid (200 mg, 0.723 mmol) and tert-butyl 3,3-dimethylpiperazine-1-carboxylate (164.3 mg, 0.767 mmol) in DMF (2.13 mL) was added N-methyl morpholine (329.2 mg, 357.8 μL, 3.26 mmol) at ambient temperature. T3P (575.4 mg, 0.904 mmol) 50% W/W in DMF was added dropwise and the solution was stirred at room temperature for 30 minutes. NaHCO₃ was added and the aqueous phase was extracted with EtOAc 3 times. The combined organic phase was washed with sat aq. ammonium chloride and brine, dried over MgSO4, filtered and evaporated under reduced pressure. The residue was purified by silica gel chromatography affording the title compound, tert-butyl 4-(7-bromo-5-chloro-furo[3,2-b]pyridine-2-carbonyl)-3,3-dimethyl-piperazine-1-carboxylate (295 mg, 0.6240 mmol, 86.26%). ESI-MS m/z calc. 471.05606, found 472.24 (M+1)+; Rt: 2.05 min using Method C.

### Step V: tert-butyl 4-[5-chloro-7-(1-cyano-3,3-difluoro-cyclobutyl)furo[3,2-b]pyridine-2-carbonyl]-3,3-dimethyl-piperazine-1-carboxylate

To an oven-dried round bottom flask containing 3,3-difluorocyclobutanecarbonitrile (475.5 mg, 399.6 μL, 4.06 mmol) in THF (2.707 mL) was added bis(2,2,6,6-tetramethyl-1-piperidyl)zinc (13.54 mL of 0.35 M, 4.74 mmol) and the solution was stirred for 20 minutes. In a separate flask were mixed rac-BINAP (105.4 mg, 0.169 mmol) and Pd(OAc)₂ (38.0 mg, 0.169 mmol) in THF (4.1 mL) and the solution was warmed to 50° C. To the catalyst mixture was added tert-butyl 4-(7-bromo-5-chloro-furo[3,2-b]pyridine-2-carbonyl)-3,3-dimethyl-piperazine-1-carboxylate (800 mg, 1.692 mmol) in THF (4 mL) and the solution of nitrile was added portionwise over 1 h. The reaction mixture was stirred at 50° C. for 3.5 h. The solution was cooled to room temperature and poured into a stirred mixture of EtOAc and 1N HCl. The phases were separated and the aqueous phase was extracted twice with EtOAc. The combined organic phase was washed with sat. NaHCO3 and brine, dried over MgSO4, filtered and the filtrate evaporated under reduced pressure. The residue was purified by silica gel chromatography affording the title compound. ESI-MS m/z calc. 508.16888, found: 453.28 (M-tBu) Rt: 3.61 minutes using Method A.

### Step VI: tert-butyl 4-[5-(4-chloro-3-fluoro-phenyl)-7-(1-cyano-3,3-difluoro-cyclobutyl)furo[3,2-b]pyridine-2-carbonyl]-3,3-dimethyl-piperazine-1-carboxylate

To a solution of tert-butyl 4-[5-chloro-7-(1-cyano-3,3-difluoro-cyclobutyl)furo[3,2-b]pyridine-2-carbonyl]-3,3-dimethyl-piperazine-1-carboxylate (750 mg, 1.47 mmol) in 1,4-dioxane (14.7 mL) was added Pd(PPh₃)₄ (85.2 mg, 0.0737 mmol), (4-chloro-3-fluoro-phenyl)boronic acid (321.2 mg, 1.84 mmol) and Na₂CO₃ (1.84 mL of 2 M, 3.68 mmol). The solution was degassed by bubbling nitrogen for 5 minutes before being stirred at 70° C. for 18 h. The solution was cooled to room temperature, water was added and the aqueous phase is extracted with EtOAc (×3). The combined organic phase was washed with 1N HCl, sat. NaHCO₃ and brine, dried over MgSO4, filtered and the filtrate evaporated under reduced pressure. The residue was purified by silica gel chromatography affording the title compound, tert-butyl 4-[5-(4-chloro-3-fluoro-phenyl)-7-(1-cyano-3,3-difluoro-cyclobutyl)furo[3,2-b]pyridine-2-carbonyl]-3,3-dimethyl-piperazine-1-carboxylate (642 mg, 1.04 mmol, 70%). ESI-MS m/z calc. 602.1908, found 603.51 (M+1)+. Retention time: 4.36 minutes using Method A.

### Step VII: 1-[5-(4-chloro-3-fluoro-phenyl)-2-(2,2-dimethylpiperazine-1-carbonyl)furo[3,2-b]pyridin-7-yl]-3,3-difluoro-cyclobutanecarbonitrile

To a solution of tert-butyl 4-[5-(4-chloro-3-fluoro-phenyl)-7-(1-cyano-3,3-difluoro-cyclobutyl)furo[3,2-b]pyridine-2-carbonyl]-3,3-dimethyl-piperazine-1-carboxylate (629 mg, 1.015 mmol) in DCM (2.239 mL) was added TFA (1.389 g, 938.5 μL, 12.18 mmol) and the solution is stirred for 3 h. The volatiles were removed under reduced pressure and the residue dissolved in EtOAc. A saturated solution of sodium bicarbonate was added and the aqueous phase was extracted with EtOAc twice. The combined organic phase was washed with brine, dried over K₂CO₃, filtered and the filtrate evaporated under reduced pressure affording the title compound which was used in the subsequent step without further purification. 1-[5-(4-chloro-3-fluoro-phenyl)-2-(2,2-dimethylpiperazine-1-carbonyl)furo[3,2-b]pyridin-7-yl]-3,3-difluoro-cyclobutanecarbonitrile (510 mg, 100%) ESI-MS m/z calc. 502.13834, found 503.41 (M+1)+; Rt: 2.52 minutes using Method A.

### Step VIII: 1-[5-(4-chloro-3-fluoro-phenyl)-2-[4-(3-hydroxy-3-methyl-cyclobutanecarbonyl)-2,2-dimethyl-piperazine-1-carbonyl]furo[3,2-b]pyridin-7-yl]-3,3-difluoro-cyclobutanecarbonitrile

According to general Method 1, 3-hydroxy-3-methyl-cyclobutanecarboxylic acid (27.2 mg, 0.209 mmol) in DMF (1 mL) was reacted with N-methylmorpholine (109 μL, 0.994 mmol) at ambient temperature. T3P (177 mg, 0.278 mmol) 50% W/W in DMF was added dropwise and the solution was stirred at room temperature for 30 minutes. Aq. NaHCO₃ was added and the aqueous phase was extracted with EtOAc 3 times. The combined organic phase was washed with sat. NH₄Cl and brine, dried over MgSO₄, filtered and the filtrate evaporated under reduced pressure. The residue was purified by silica gel chromatography and by reverse phase preparative HPLC affording the title compound as a white solid. 1-[5-(4-chloro-3-fluoro-phenyl)-2-[4-(3-hydroxy-3-methyl-cyclobutanecarbonyl)-2,2-dimethyl-piperazine-1-carbonyl]furo[3,2-b]pyridin-7-yl]-3,3-difluoro-cyclobutanecarbonitrile (58.8 mg, 47%) ¹H NMR (400 MHz, cdcl3) δ 7.87 (d, J=10.0 Hz, 1H), 7.77-7.63 (m, 2H), 7.54 (t, J=7.7 Hz, 1H), 7.49 (s, 1H), 3.98-3.84 (m, 2H), 3.81-3.43 (m, 8H), 2.96-2.83 (m, 1H), 2.45-2.30 (m, 4H), 1.62 (d, J=4.1 Hz, 6H), 1.41 (d, J=10.3 Hz, 3H). ESI-MS m/z calc. 614.1908, found 615.5 (M+1)+; Rt: 3.37 minutes using Method A.

### Example 64: 1-(tert-butyl)-4-(7-(tert-butyl)-5-(4-fluorophenyl)furo[3,2-b]pyridine-2-carbonyl)-3,3-dimethylpiperazin-2-one

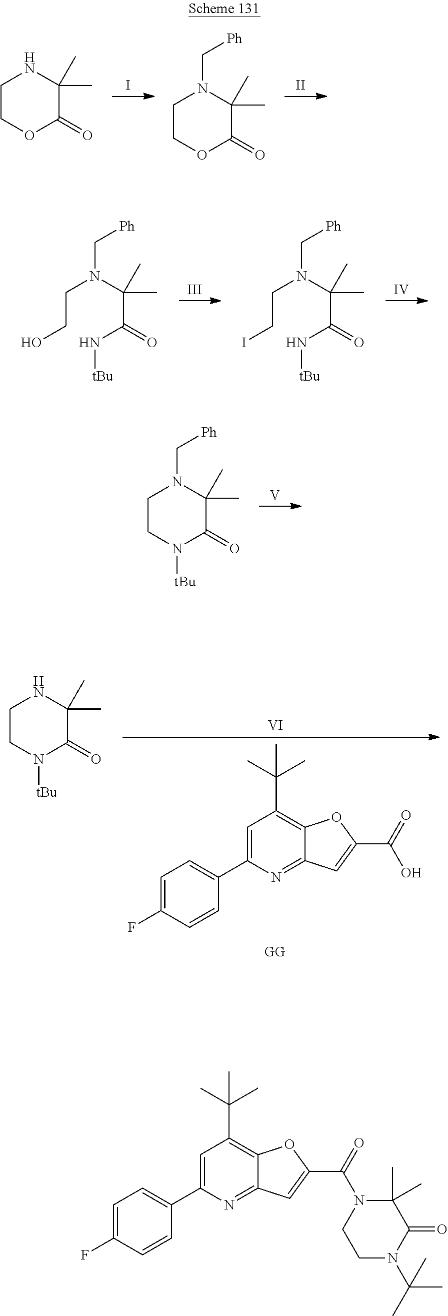

### Step I: 4-benzyl-3,3-dimethylmorpholin-2-one

3,3-dimethylmorpholin-2-one (500 mg, 3.871 mmol) and anhydrous potassium carbonate (1.873 g, 13.55 mmol) in dry DMF (6.110 mL) was stirred for 30 min at room temperature. BnBr (761.4 mg, 529.5 μL, 4.45 mmol) was added dropwise into the reaction mixture and heated at 65° C. for 18 h. The reaction mixture was cooled to room temperature and quenched with water (25 mL). EtOAc was added and the phases were separated. The organic phase was washed twice with water and brine (1:1 mixture), dried over MgSO4, filtered and evaporated under reduced pressure. The residue was purified using a 40 g silica gel Redisep Isco column and a gradient of 5 to 50% EtOAc/Hexanes in 18 CV to afford 4-benzyl-3,3-dimethyl-morpholin-2-one (376 mg, 44%) 1H NMR (400 MHz, DMSO-d6) δ 7.39-7.29 (m, 4H), 7.28-7.20 (m, 1H), 4.21-4.13 (m, 2H), 3.59 (s, 2H), 2.68-2.58 (m, 2H), 1.43 (s, 6H). ESI-MS m/z calc. 219.12593, found 220.32 (M+1)+.

### Step II: 2-(benzyl(2-hydroxyethyl)amino)-N-(tert-butyl)-2-methylpropanamide

AlCl₃ (468.2 mg, 191.9 μL, 3.511 mmol) in DCM (2.8 mL) was cooled in an ice-water bath. Et₃N (533.0 mg, 734.2 μL, 5.267 mmol) was added dropwise. The mixture was stirred for an additional 15 min and then warmed to 25° C. A solution of 4-benzyl-3,3-dimethyl-morpholin-2-one (350 mg, 1.60 mmol) in DCM (200 μL) and t-butylamine (1.167 g. 1.67 mL, 15.96 mmol) were added dropwise to this mixture which was stirred at rt for 18 h (ON). A solution of Na₂CO₃ (2M) was added to quench the reaction followed by water and DCM. The phases were separated and the aqueous phase was extracted once more with DCM and once with EtOAc. The organic phases were combined, dried over MgSO₄, filtered and evaporated under reduced pressure to afford 2-[benzyl(2-hydroxyethyl)amino]-N-tert-butyl-2-methyl-propanamide (467 mg, 100%).

### Step III: 2-(benzyl(2-iodoethyl)amino)-N-(tert-butyl)-2-methylpropanamide

A mixture of 2-[benzyl(2-hydroxyethyl)amino]-N-tert-butyl-2-methyl-propanamide (467 mg, 1.60 mmol), imidazole (391.4 mg, 5.75 mmol) and triphenylphosphine (1.089 g, 962.0 μL, 4.15 mmol) in toluene (16.55 mL) was added iodine (1.054 g. 213.8 μL, 4.15 mmol) and the reaction mixture was stirred at 85° C. for 18 h. Water was added and the organic phase was extracted with EtOAc. The extracted organic phase was washed with a solution of Na₂S₂O₃ before being dried over MgSO₄, filtered and evaporated under reduced pressure. A white solid containing O═PPh3 was obtained. Crude reaction mixture was purified using a 40 g silica gel Redisep Isco colomn and a gradient of 5 to 20%0/EtOAc/Hexanes in 15 CV to afford 2-[benzyl(2-iodoethyl)amino]-N-tert-butyl-2-methyl-propanamide (487 mg, 76%). 1H NMR (400 MHz, DMSO-d6) δ 7.46-6.95 (m, 6H), 3.61-3.51 (m, 2H), 2.95-2.86 (m, 2H), 2.84-2.73 (m, 2H), 1.32-1.23 (m, 9H), 1.22-1.10 (m, 6H). ESI-MS m/z calc. 402.11682, found 402.99 (M+1)+.

### Step IV: 4-benzyl-1-(tert-butyl)-3,3-dimethylpiperazin-2-one

Under a nitrogen atmosphere, 2-[benzyl(2-iodoethyl)amino]-N-tert-butyl-2-methyl-propanamide (487 mg, 1.210 mmol) was dissolved in DMF (24.21 mL) and NaH (53.23 mg, 1.33 mmol) is added portion-wise. The reaction mixture was stirred at rt for 30 minutes. The reaction was treated with sat. NH₄Cl followed by water and the resulting mixture was allowed to stir at rt for 30 minutes. The solid was collected and dried in vacuo overnight to afford 4-benzyl-1-tert-butyl-3,3-dimethyl-piperazin-2-one (290 mg, 1.057 mmol, 87.35%). 1H NMR (400 MHz, DMSO-d6) δ 7.37-7.27 (m, 4H), 7.26-7.18 (m, 1H), 3.52 (s, 2H), 3.17-3.07 (m, 2H), 2.54-2.43 (m, 2H), 1.33 (s, 9H), 1.27 (s, 6H). ESI-MS m z calc. 274.2045, found 275.42 (M+1).

### Step V: 1-(tert-butyl)-3,3-dimethylpiperazin-2-one

To a solution of 4-benzyl-1-tert-butyl-3,3-dimethyl-piperazin-2-one (290 mg, 1.06 mmol) in EtOAc (2.114 mL) was added Pd—C (11.25 mg, 0.106 mmol). The mixture was hydrogenated using a balloon for 4.5 h. The reaction was filtered on celite, washed with EtOAc and the solvent was removed under reduced pressure to afford 1-tert-butyl-3,3-dimethyl-piperazin-2-one (176 mg, 90%). 1H NMR (400 MHz, Chloroform-d) δ 3.40-3.30 (m, 2H), 3.07-2.95 (m, 2H), 1.42 (s, 9H), 1.37 (s, 6H). ESI-MS m/z calc. 184.15756, found 185.37 (M+1)+.

### Step VI. 1-(tert-butyl)-4-(7-(tert-butyl)-5-(4-fluorophenyl)furo[3,2-b]pyridine-2-carbonyl)-3,3-dimethylpiperazin-2-one

According to General Procedure 1, 7-tert-butyl-5-(4-fluorophenyl)furo[3,2-b]pyridine-2-carboxylic acid (50 mg, 0.1554 mmol) (intermediate GG) in DMF (0.5 mL) containing DIPEA (94.7 μL, 0.544 mmol) and 1-tert-butyl-3,3-dimethyl-piperazin-2-one (31.5 mg, 0.171 mmol) was treated with HATU (88.6 mg, 0.233 mmol). The reaction mixture was stirred at RT for 2 h and partitioned between H₂O and EtOAc. The organic phase was washed (water, brine), dried (MgSO₄), filtered and evaporated under reduced pressure. The residue was purified by preparative HPLC (MeCN—H₂O-formic acid) to afford 1-tert-butyl-4-[7-tert-butyl-5-(4-fluorophenyl)furo[3,2-b]pyridine-2-carbonyl]-3,3-dimethyl-piperazin-2-one (43.6 mg, 58%). ¹H NMR (400 MHz, DMSO-d6) δ 8.20-8.13 (m, 2H), 7.76 (s, 1H), 7.59 (s, 1H), 7.38-7.28 (m, 2H), 3.80-3.72 (m, 2H), 3.64-3.57 (m, 2H), 1.67 (s, 6H), 1.53 (s, 9H), 1.39 (s, 9H). ESI-MS m/z calc. 479.25842, found 480.22 (M+1)+; Retention time: 4.72 min using method A.

### Example 65. Preparation of 4-[7-(1-ethylpropyl)-5-(4-fluorophenyl)furo[3,2-b]pyridine-2-carbonyl]-3,3-dimethyl-1-(1H-pyrazol-4-yl)piperazin-2-one

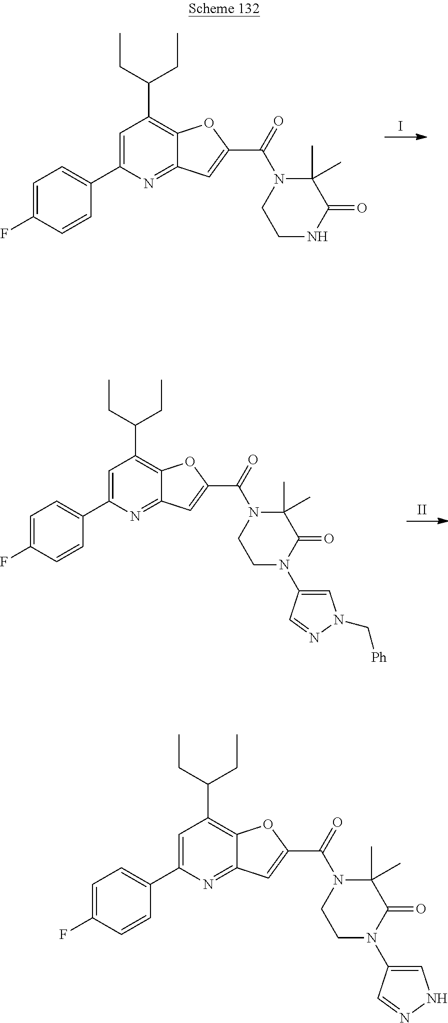

### Step I: 1-(1-benzylpyrazol-4-yl)-4-[7-(1-ethylpropyl)-5-(4-fluorophenyl)furo[3,2-b]pyridine-2-carbonyl]-3,3-dimethyl-piperazin-2-one

To a solution of 4-[7-(1-ethylpropyl)-5-(4-fluorophenyl)furo[3,2-b]pyridine-2-carbonyl]-3,3-dimethyl-piperazin-2-one (70 mg, 0.160 mmol) in DMF (1.750 mL) were added 1-benzyl-4-iodo-pyrazole (68.18 mg, 0.240) mmol), CuI (15.23 mg, 0.080 mmol) and K₃PO₄ (67.93 mg, 0.320 mmol). The mixture was degassed for 10 min, then to it was added N,N′-dimethylethane-1,2-diamine (14 mg, 16.91 μL, 0.159 mmol). The mixture was allowed to stir overnight at 100° C. The suspension was cooled down, water was added along with EtOAc. The phases were separated to afford a crude mixture of 1-(1-benzylpyrazol-4-yl)-4-[7-(1-ethylpropyl)-5-(4-fluorophenyl)furo[3,2-b]pyridine-2-carbonyl]-3,3-dimethyl-piperazin-2-one (95.0 mg, 100.0%).

### Step II: 4-[7-(1-ethylpropyl)-5-(4-fluorophenyl)furo[3,2-b]pyridine-2-carbonyl]-3,3-dimethyl-1-(1H-pyrazol-4-yl)piperazin-2-one

To a solution of 1-(1-benzylpyrazol-4-yl)-4-[7-(1-ethylpropyl)-5-(4-fluorophenyl)furo[3,2-b]pyridine-2-carbonyl]-3,3-dimethyl-piperazin-2-one (18.8 mg, 0.0317 mmol) in EtOH (376.0 μL) was added 20% Pd(OH)₂ (4.447 mg, 0.00633 mmol). The mixture was hydrogenated using a H₂ balloon and stirred at RT for 48 h. The mixture was filtered on celite and purified by preparative HPLC to give 4-[7-(1-ethylpropyl)-5-(4-fluorophenyl)furo[3,2-b]pyridine-2-carbonyl]-3,3-dimethyl-1-(1H-pyrazol-4-yl)piperazin-2-one (2 mg, 12%). ESI-MS m/z calc. 503.23328, found 504.53 (M+1)+. 1H NMR (400 MHz, DMSO-d6) δ 12.80 (s, 1H), 8.22-8.12 (m, 2H), 8.03 (s, 1H), 7.85 (s, 1H), 7.73 (s, 1H), 7.60 (s, 1H), 7.35-7.22 (m, 2H), 3.93 (s, 4H), 2.95 (m, 1H), 2.04 (s, 2H), 1.94-1.77 (m, 4H), 1.76 (s, 6H), 0.77 (t, J=7.4 Hz, 6H).

### Example 66. 4-[7-(1-ethylpropyl)-5-(4-fluorophenyl)furo[3,2-b]pyridine-2-carbonyl]-3,3-dimethyl-2-oxo-piperazine-1-carbonitrile

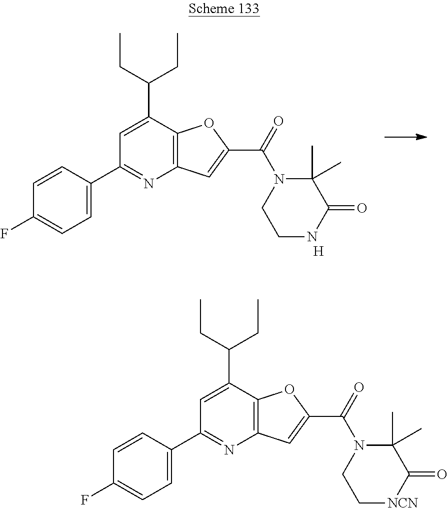

To a solution of 4-[7-(1-ethylpropyl)-5-(4-fluorophenyl)furo[3,2-b]pyridine-2-carbonyl]-3,3-dimethyl-piperazin-2-one (70 mg, 0.16 mmol) in DMF (1.4 mL) was added NaH (60% dispersion in mineral oil, 10.9 mg, 0.272 mmol) at 0° C. and the solution was allowed to stir for 10 min. Cyanogen bromide (28.8 mg, 27.71 μL, 0.272 mmol) at 0° C. and the mixture was allowed to warm to RT and stirred for 7 h. The volatiles were removed under reduced pressure and the residue was purified by Preparative-HPLC (MeCN—H2O-formic acid) to give 4-[7-(1-ethylpropyl)-5-(4-fluorophenyl)furo[3,2-b]pyridine-2-carbonyl]-3,3-dimethyl-2-oxo-piperazine-1-carbonitrile (3 mg, 4%). ESI-MS m/z calc. 462.20673, found 463.47 (M+1)+; Rt: 1.98 minutes, using method C. ¹H NMR (400 MHz, DMSO-d6) δ 8.22-8.09 (m, 2H), 7.86 (s, 1H), 7.59 (s, 1H), 7.36-7.24 (m, 2H), 4.15-4.02 (m, 2H), 3.94 (t, J=4.8 Hz, 2H), 2.97-2.81 (m, 1H), 1.88-1.77 (m, 4H), 1.77 (s, 6H), 0.76 (t, J=7.4 Hz, 6H).

### Example 67: Preparation of cis-4-[4-(7-tert-butyl-5-spiro[3.3]heptan-2-yl-furo[3,2-b]pyridine-2-carbonyl)-3,3-dimethyl-piperazine-1-carbonyl]-1-methyl-cyclohexanecarboxylic acid and trans-4-[4-(7-tert-butyl-5-spiro[3.3]heptan-2-yl-furo[3,2-b]pyridine-2-carbonyl)-3,3-dimethyl-piperazine-1-carbonyl]-1-methyl-cyclohexanecarboxylic acid

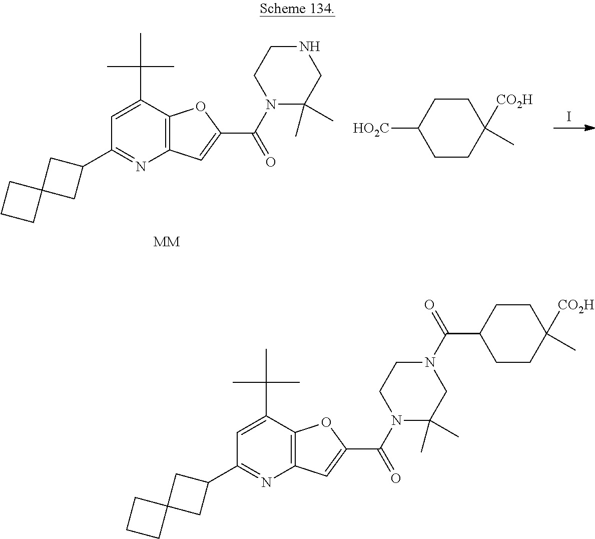

A mixture of intermediate MM (30) mg, 0.733 mmol) and 1-methylcyclohexane-1,4-dicarboxylic acid (204.6 mg, 1.10 mmol) in DMF (5 mL) was treated with DIPEA (568.0 mg, 765.5 μL, 4.40 mmol) and T3P (559.4 μL of 50% w/v, 0.879 mmol). The resulting clear light brown solution was stirred at room temp overnight. The reaction mixture was diluted with water (5 mL) and 5% citric acid (5 mL), extracted twice with EtOAc. Organic layers dried (MgSO₄), filtered and concentrated in vacuo. The residue was purified by preparative HPLC to give two fractions.

Fraction 1: cis-4-[4-(7-tert-butyl-5-spiro[3.3]heptan-2-yl-furo[3,2-b]pyridine-2-carbonyl)-3,3-dimethyl-piperazine-1-carbonyl]-1-methyl-cyclohexanecarboxylic acid. ESI-MS m/z calc. 577.35156, found 578.0 (M+1)+; Rt: 3.86 minutes using Method A.

Fraction 2: trans-4-[4-(7-tert-butyl-5-spiro[3.3]heptan-2-yl-furo[3,2-b]pyridine-2-carbonyl)-3,3-dimethyl-piperazine-1-carbonyl]-1-methyl-cyclohexanecarboxylic acid. ESI-MS m/z calc. 577.35156, found 578.0 (M+1)+; Rt: 3.63 minutes using Method A.

### Example 67

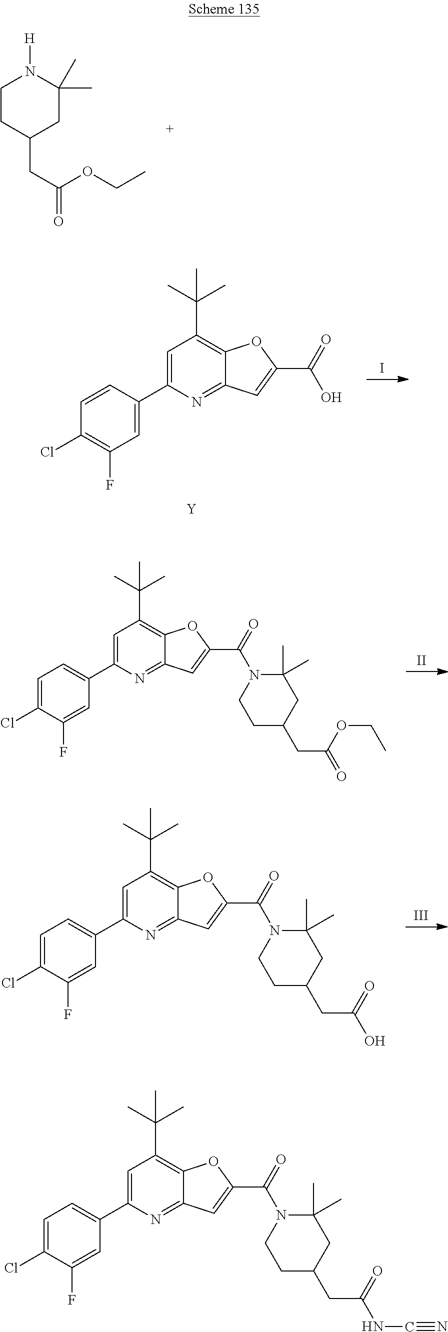

### Step I: ethyl 2-[1-[7-tert-butyl-5-(4-chloro-3-fluoro-phenyl)furo[3,2-b]pyridine-2-carbonyl]-2,2-dimethyl-4-piperidyl]acetate

To a solution of Intermediates Y (206.5 mg, 0.594 mmol) and GGG (140 mg, 0.594 mmol) in 3.85 DMF (1.4 mL) was added DIPEA (517 μL, 2.97 mmol) and HATU (248.8 mg, 0.654 mmol). The reaction mixture was then diluted with water, extracted three times with EtOAc, dried over sodium sulfate, filtered, and the solvent was removed under reduced pressure. The residue was then purified by silica gel chromatography eluting with 0 to 20% EtOAc/Hexanes affording the title compound, ethyl 2-[1-[7-tert-butyl-5-(4-chloro-3-fluoro-phenyl)furo[3,2-b]pyridine-2-carbonyl]-2,2-dimethyl-4-piperidyl]acetate (280 mg, 89%). 1H NMR (400 MHz, Chloroform-d) δ 7.78 (d, J=2.0 Hz, 1H), 7.66 (ddd, J=8.5, 2.1, 0.8 Hz, 1H), 7.50 (s, 1H), 7.40 (dd, J=8.4, 7.5 Hz, 1H), 7.33 (s, 1H), 4.21-4.06 (m, 2H), 3.85-3.76 (m, 1H), 3.33-3.18 (m, 1H), 2.24 (s, 2H), 2.21-2.12 (m, 1H), 1.93-1.84 (m, 1H), 1.60 (s, 3H), 1.56 (d, J=1.6 Hz, 1H), 1.50 (s, 9H), 1.47 (s, 3H), 1.43 (s, 1H), 1.35 (dd, J=12.4, 3.0 Hz, 1H), 1.22 (t, J=7.1 Hz, 3H). ESI-MS m/z calc. 528.2191, found 529.41 (M+1)+; Retention time: 2.46 minutes (method A).

### Step II: 2-[1-[7-tert-butyl-5-(4-chloro-3-fluoro-phenyl)furo[3,2-b]pyridine-2-carbonyl]-2,2-dimethyl-4-piperidyl]acetic acid

Ethyl 2-[1-[7-tert-butyl-5-(4-chloro-3-fluoro-phenyl)furo[3,2-b]pyridine-2-carbonyl]-2,2-dimethyl-4-piperidyl]acetate (280 mg, 0.529 mmol) was dissolved in MeOH (7 mL) and NaOH (5.29 mL of 1 M, 5.29 mmol) was added. The mixture was stirred at r.t. for one day. The reaction mixture was extracted with diethyl ether, then brought to pH=3 using citric acid. The aqueous phase was extracted three times with EtOAc, and the combined organic extracts were washed with water, brine, and dried over sodium sulfate and the solvent was removed under reduced pressure. The residue was then taken up in MeCN/water and put on the lyophilizer for one day to afford 2-[1-[7-tert-butyl-5-(4-chloro-3-fluoro-phenyl)furo[3,2-b]pyridine-2-carbonyl]-2,2-dimethyl-4-piperidyl]acetic acid (204 mg, 74%). 1H NMR (400 MHz, DMSO-d6) δ 12.11 (s, 1H), 8.14 (dd, J=11.0, 2.1 Hz, 1H), 8.00 (ddd, J=8.5, 2.1, 0.7 Hz, 1H), 7.79 (s, 1H), 7.68 (dd, J=8.5, 7.8 Hz, 1H), 7.46 (s, 1H), 3.84-3.66 (m, 1H), 3.27-3.18 (m, 1H), 2.19 (d, J=6.9 Hz, 2H), 2.09 (s, 1H), 1.86 (d, J=12.4 Hz, 1H), 1.61-1.54 (m, 1H), 1.51 (s, 3H), 1.50 (s, 9H), 1.45 (s, 1H), 1.41 (s, 3H), 1.35-1.26 (m, 1H). ESI-MS m/z calc. 500.1878, found 501.45 (M+1)+; Retention time: 2.02 minutes (method A).

### Step III: 2-[1-[7-tert-butyl-5-(4-chloro-3-fluoro-phenyl)furo[3,2-b]pyridine-2-carbonyl]-2,2-dimethyl-4-piperidyl]-N-cyano-acetamide

To a solution of (cyanoamino) sodium (5.37 mg, 0.0838 mmol) and 2-[l-[7-tert-butyl-5-(4-chloro-3-fluoro-phenyl)furo[3,2-b]pyridine-2-carbonyl]-2,2-dimethyl-4-piperidyl]acetic acid (42 mg, 0.0838 mmol) in dry DMF (1.3 mL) was added DIPEA (73.0 μL, 0.419 mmol) and HATU (38.3 mg, 0.101 mmol). The reaction mixture was diluted with water, extracted three times with EtOAc, dried over sodium sulfate, filtered, and the solvent was removed under reduced pressure. The reaction mixture was directly submitted for reverse phase prep HPLC to afford after lyophilizaton 2-[1-[7-tert-butyl-5-(4-chloro-3-fluoro-phenyl)furo[3,2-b]pyridine-2-carbonyl]-2,2-dimethyl-4-piperidyl]-N-cyano-acetamide (12 mg, 53%). 1H NMR (400 MHz, DMSO-d6) δ 8.17 (dd, J=11.0, 2.1 Hz, 1H), 8.03 (ddd, J=8.5, 2.1, 0.7 Hz, 1H), 7.82 (s, 1H), 7.72 (dd, J=8.5, 7.8 Hz, 1H), 7.49 (s, 1H), 3.83-3.67 (m, 1H), 3.26-3.18 (m, 1H), 2.27 (d, J=6.7 Hz, 2H), 2.17 (s, 1H), 1.91-1.81 (m, 1H), 1.58 (dd, J=14.2, 3.9 Hz, 1H), 1.54 (s, 3H), 1.53 (s, 9H), 1.47 (s, 1H), 1.44 (s, 3H), 1.37-1.26 (m, 1H). ESI-MS m/z calc. 524.19904, found 525.5 (M+1)+; Retention time: 4.15 minutes (method A).

### Example 68: 2-[1-[7-tert-butyl-5-(4-chloro-3-fluoro-phenyl)furo[3,2-b]pyridine-2-carbonyl]-2,2-dimethyl-4-piperidyl]-N-(trifluoromethylsulfonyl)acetamide

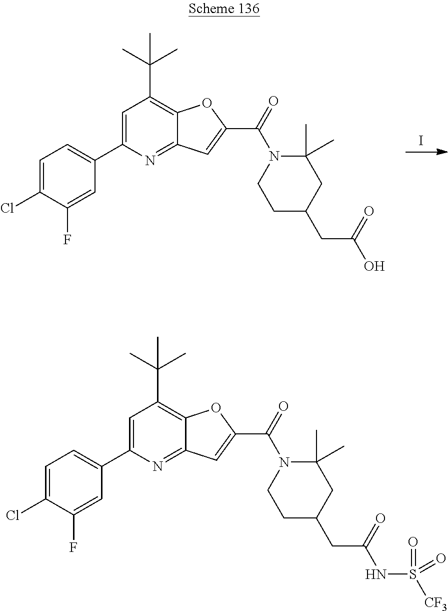

### Step I

To a solution of 2-[1-[7-tert-butyl-5-(4-chloro-3-fluoro-phenyl)furo[3,2-b]pyridine-2-carbonyl]-2,2-dimethyl-4-piperidyl]acetic acid (30 mg, 0.060 mmol) and trifluoromethanesulfonamide (8.9 mg, 0.060 mmol) in dry DMF (683.7 μL) was added DIPEA (52.2 μL, 0.299 mmol) and HATU (25.1 mg, 0.0659 mmol). The reaction mixture was stirred 30 min, then diluted with water, extracted three times with EtOAc, dried over sodium sulfate, filtered, and the solvent was removed under reduced pressure. The residue was then purified by silica gel chromatography on a 10 g column eluting with 50 to 100% EtOAc/Hexanes affording the title compound, 2-[1-[7-tert-butyl-5-(4-chloro-3-fluoro-phenyl)furo[3,2-b]pyridine-2-carbonyl]-2,2-dimethyl-4-piperidyl]-N-(trifluoromethylsulfonyl)acetamide (25 mg, 0.0389 mmol, 65.1%). 1H NMR (400 MHz, DMSO-d6) δ 8.17 (dd, J=11.0, 2.1 Hz, 1H), 8.03 (ddd, J=8.5, 2.1, 0.7 Hz, 1H), 7.81 (s, 1H), 7.71 (dd, J=8.5, 7.8 Hz, 1H), 7.47 (s, 1H), 3.80-3.69 (m, 1H), 3.27-3.16 (m, 1H), 2.16-2.07 (m, 1H), 2.04-1.97 (m, 2H), 1.91-1.81 (m, 1H), 1.63-1.57 (m, 1H), 1.53 (d, J=1.5 Hz, 12H), 1.43 (s, 3H), 1.41-1.33 (m, 1H), 1.32-1.21 (m, 1H). ESI-MS m/z calc. 631.1531, found 632.43 (M+1)+; Retention time: 2.81 minutes (method A).

### Example 69: [7-tert-butyl-5-[(2S)-2-(trifluoromethyl)pyrrolidin-1-yl]furo[3,2-b]pyridin-2-yl]-[4-(3-hydroxy-3-methyl-cyclobutanecarbonyl)-2,2-dimethyl-piperazin-1-yl]methanone

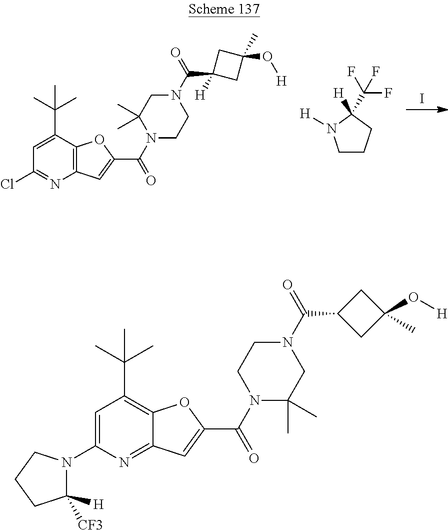

### Step 1

To a solution of (2S)-2-(trifluoromethyl)pyrrolidine (200 μL of 0.5 M, 0.100 mmol) and NaOtBu (18.3 mg, 0.191 mmol) was added a solution of [2-(2-aminophenyl)phenyl]-chloro-palladium;dicyclohexyl-[2-(2,4,6-triisopropylphenyl)phenyl]phosphane (3.8 mg, 0.0048 mmol), (7-tert-butyl-5-chloro-furo[3,2-b]pyridin-2-yl)-[4-(3-hydroxy-3-methyl-cyclobutanecarbonyl)-2,2-dimethyl-piperazin-1-yl]methanone (44 mg, 0.095 mmol) and X-Phos (2.3 mg, 0.0048 mmol) in dioxane (0.8 mL) and the solution was stirred at 90° C. for 1 h. The solution was cooled to room temperature, acidified with 12N HCl, basified to pH 8 using sat. aq. NaHCO₃ and diluted with DCM. The phases were separated using an Isolute Phase Separator and the aq. phase washed with DCM. The combined organic phase was treated with Silicycle's Siliamet S metal scavenver overnight, filtered and the filtrate was evaporated under reduced pressure. The product was purified by reverse phase preparative HPLC affording [7-tert-butyl-5-[(2S)-2-(trifluoromethyl)pyrrolidin-1-yl]furo[3,2-b]pyridin-2-yl]-[4-(3-hydroxy-3-methyl-cyclobutanecarbonyl)-2,2-dimethyl-piperazin-1-yl]methanone (7.3 mg, 14%). ¹H NMR (400 MHz, dmso) δ 7.24 (d, J=12.1 Hz, 1H), 6.59 (d, J=2.0 Hz, 1H), 5.19-5.06 (m, 1H), 5.00 (d, J=18.4 Hz, 1H), 3.92-3.84 (m, 2H), 3.79-3.69 (m, 1H), 3.65-3.59 (m, 2H), 3.53 (s, 1H), 3.51-3.45 (m, 2H), 2.94-2.76 (m, 1H), 2.20-2.04 (m, 7H), 1.52-1.38 (m, 15H), 1.27 (d, J=10.8 Hz, 3H). ESI-MS m/z calc. 564.29236, found 565.59 (M+1)+; Retention time: 3.42 minutes (method A).

### Example 70: [7-tert-butyl-5-(4-chlorophenyl)furo[3,2-b]pyridin-2-yl]-[2,2-dimethyl-4-[6-(1H-tetrazol-5-yl)-2-pyridyl]piperazin-1-yl]methanone

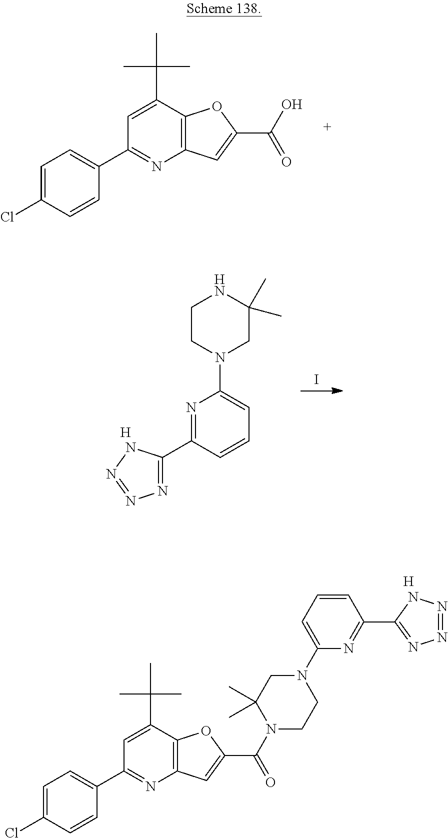

### Step I

3,3-dimethyl-1-[6-(1H-tetrazol-5-yl)-2-pyridyl]piperazine (intermediate TT, 51.8 mg, 0.1872 mmol) is dissolved in DMF (700 μL), 7-tert-butyl-5-(4-chlorophenyl)furo[3,2-b]pyridine-2-carboxylic acid (intermediate V, 61.7 mg, 0.1871 mmol) was added followed by HATU (85.5 mg, 0.2249 mmol) and DIPEA (97.8 μL, 0.5615 mmol) and the mixture was stirred at room temperature overnight. The crude reaction mixture was purified by reverse phase chromatography using a 20 to 90% ACN in water gradient to afford [7-tert-butyl-5-(4-chlorophenyl)furo[3,2-b]pyridin-2-yl]-[2,2-dimethyl-4-[6-(1H-tetrazol-5-yl)-2-pyridyl]piperazin-1-yl]methanone (53.3 mg, 0.09287 mmol, 49.61%). 1H NMR (400 MHz, DMSO-d6) δ 8.13 (d, J=8.6 Hz, 2H), 7.75 (s, 1H), 7.69 (dd, J=8.6, 7.3 Hz, 1H), 7.56-7.51 (m, 3H), 7.37 (d, J=7.2 Hz, 1H), 6.69 (d, J=8.6 Hz, 1H), 4.08 (d, J=5.5 Hz, 4H), 3.63 (t, J=5.7 Hz, 2H), 1.52 (m, 15H). ESI-MS m/z calc. 570.2258, found 571.5 (M+1)+; Retention time: 4.3 minutes (method A).

### Example 71: [7-tert-butyl-5-(4,4-dimethylcyclohexyl)furo[3,2-b]pyridin-2-yl]-[2,2-dimethyl-4-[5-(1H-tetrazol-5-yl)pyrimidin-2-yl]piperazin-1-yl]methanone

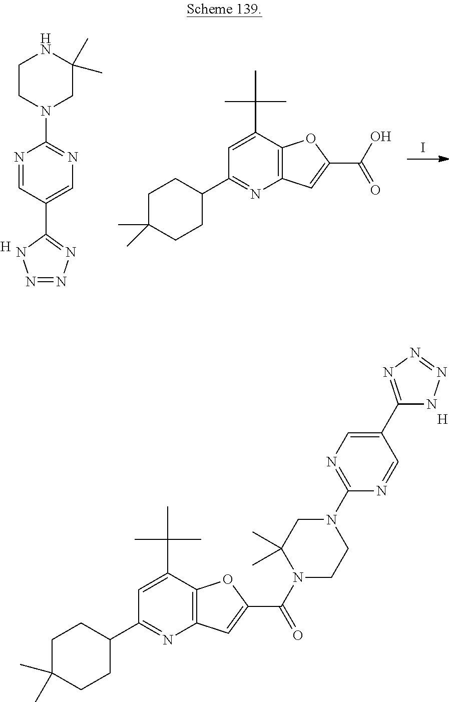

### Step I

2-(3,3-dimethylpiperazin-1-yl)-5-(1H-tetrazol-5-yl)pyrimidine (Hydrochloric Acid (1)) (intermediate SS, 55.6 mg, 0.187 mmol) was dissolved in DMSO (1.1 mL), 7-tert-butyl-5-(4,4-dimethylcyclohexyl)furo[3,2-b]pyridine-2-carboxylic acid (intermediate II, 61.6 mg, 0.187 mmol) was added followed by HATU (85.4 mg, 0.225 mmol) and DIPEA (114 μL, 0.655 mmol) and the mixture was stirred at room temperature for three hours. The reaction mixture was diluted with 2 mL of water and purified by reverse phase chromatography, using 0 to 30% water in ACN gradient to afford [7-tert-butyl-5-(4,4-dimethylcyclohexyl)furo[3,2-b]pyridin-2-yl]-[2,2-dimethyl-4-[5-(1H-tetrazol-5-yl)pyrimidin-2-yl]piperazin-1-yl]methanone (51 mg, 47%) 1H NMR (400 MHz, Methanol-d4) δ 8.95 (s, 2H), 7.33 (s, 1H), 7.22 (s, 1H), 4.18-4.07 (m, 4H), 3.90 (t, J=5.7 Hz, 2H), 2.74 (tt, J=12.1, 3.8 Hz, 1H), 1.90-1.69 (m, 6H), 1.63 (s, 6H), 1.54 (s, 9H), 1.46-1.36 (m, 2H), 1.04 (s, 3H), 0.97 (s, 3H). ESI-MS m/z calc. 571.3383, found 571.7 (M+1)+; Retention time: 4.17 minutes (method A).

### Example 72: Preparation of 4-[7-tert-butyl-5-(4-chlorophenyl)furo[3,2-b]pyridine-2-carbonyl]-1-(2,4-dimethyloxazol-5-yl)-3,3-dimethyl-piperazin-2-one

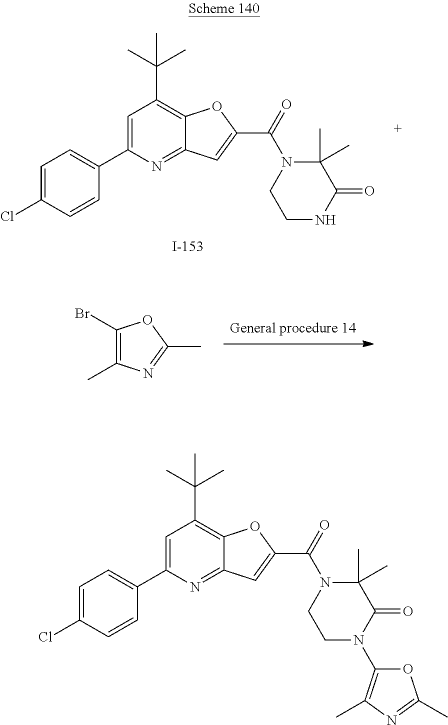

According to General Procedure 14, I-153 was treated with 60% NaH (1.95 mg, 0.0487 mmol) was added to a stirred mixture of 4-[7-tert-butyl-5-(4-chlorophenyl)furo[3,2-b]pyridine-2-carbonyl]-3,3-dimethyl-piperazin-2-one (20 mg, 0.0443 mmol) in DMF (250 μL). The mixture was stirred for 30 minutes at rt and then, a solution of 5-bromo-2,4-dimethyl-oxazole (11.7 mg, 0.0664 mmol) in DMF (150 μL) was added dropwise. The reaction mixture was stirred for overnight at 60° C. and then quenched by adding water (precipitation of the product). Aqueous was extracted twice with ethyl acetate. The combined organics layers were washed with brine solution, dried over Na₂SO₄, filtered and evaporated in vacuo. The residue was diluted in NMP and was purified by mass-directed reverse phase HPLC to afford upon lyophilization the desired 4-[7-tert-butyl-5-(4-chlorophenyl)furo[3,2-b]pyridine-2-carbonyl]-1-(2,4-dimethyloxazol-5-yl)-3,3-dimethyl-piperazin-2-one. 1H NMR (400 MHz, DMSO-d6) δ 8.20-8.13 (m, 2H), 7.80 (s, 1H), 7.65 (s, 1H), 7.61-7.53 (m, 2H), 4.09-4.02 (m, 2H), 3.89-3.81 (m, 2H), 2.35 (s, 3H), 1.94 (s, 3H), 1.81 (s, 6H), 1.53 (s, 9H). MS m/z calc. 534.20337, found 535.47 (M+1).

### Example 73: Preparation of 6-[4-[7-tert-butyl-5-(4-chlorophenyl)furo[3,2-b]pyridine-2-carbonyl]-3,3-dimethyl-piperazin-1-yl]-5-methyl-pyridine-2-carboxylic acid

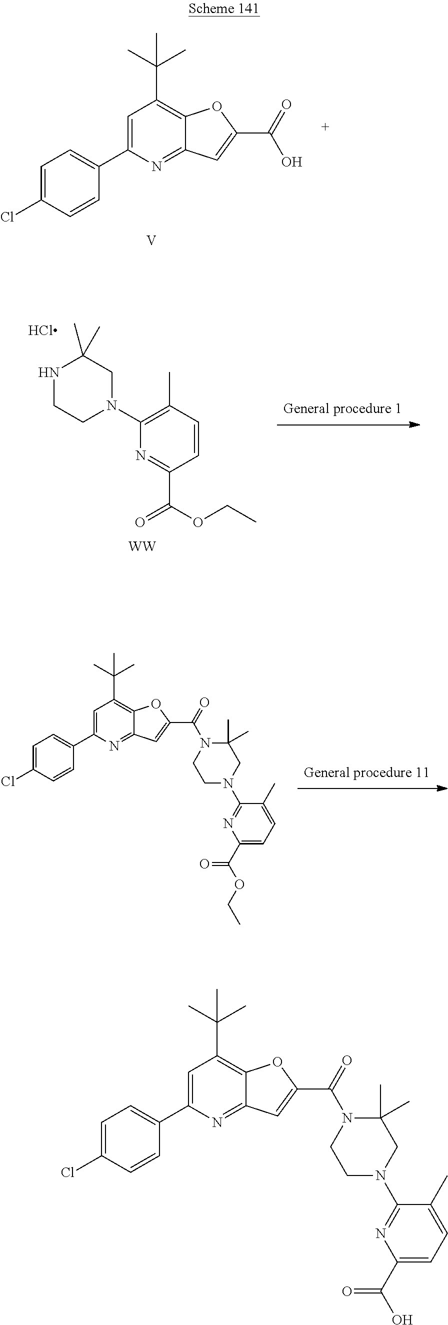

### Step I

The product was prepared according to General Procedure 1 using Intermediates V (27.4 mg, 0.0829 mmol) and WW (27.4 mg, 0.0829 mmol) in dry DMF (460 μL) using DIPEA (53.6 mg, 72 μL, 0.415 mmol) and HATU (31.5 mg, 0.0829 mmol) to afford crude ethyl ester (48.8 mg, 0.0829 mmol, quantitative).

### Step II

The ester intermediate was then hydrolyzed using General Procedure 11. A solution of the crude ethyl ester in dioxane (460 μL) was treated with NaOH (124.4 μL of 2 M, 0.249 mmol) and heated at 80° C. for 2 h. The reaction mixture was cooled down to rt and water is added. Aqueous was extracted with diethyl ether, then acidified to pH 3 using aqueous 3N HCl and, extracted three times with EtOAc. The combined organic extracts were washed with water, then brine, dried over sodium sulfate, filtered, and the solvent was removed under reduced pressure. The result compound was dissolved in DMSO and purified by mass-directed reverse-phase preparative HPLC to afford upon lyophilization the desired 6-[4-[7-tert-butyl-5-(4-chlorophenyl)furo[3,2-b]pyridine-2-carbonyl]-3,3-dimethyl-piperazin-1-yl]-5-methyl-pyridine-2-carboxylic acid (7 mg, 15%). 1H NMR (400 MHz, DMSO-d6) δ 12.80 (s, 1H), 8.16 (d, J=8.6 Hz, 2H), 7.78 (s, 1H), 7.67-7.63 (m, 1H), 7.59-7.50 (m, 4H), 4.05-3.84 (m, 2H), 3.50 (dd, J=6.4, 4.0 Hz, 2H), 3.37 (s, 2H), 2.36 (s, 3H), 1.60 (s, 6H), 1.54 (s, 9H). ESI-MS m/z calc. 560.21906. found 563.22 (M+1)+.

### Example 74: 6-[4-[7-tert-butyl-5-(4-chloro-3-fluoro-phenyl)furo[3,2-b]pyridine-2-carbonyl]-3,3-dimethyl-2-oxo-piperazin-1-yl]pyridine-3-carboxylic acid

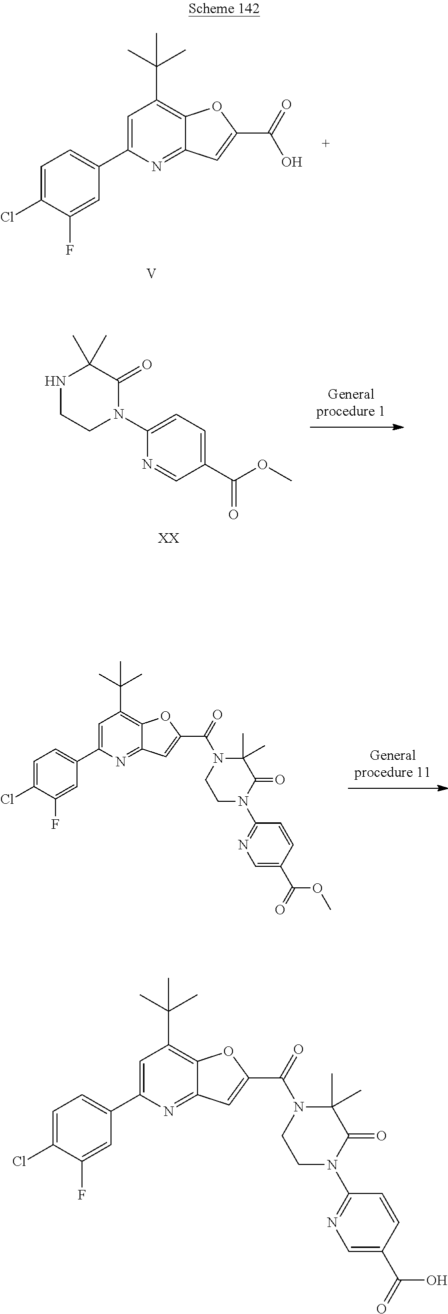

### Step 1

Intermediate V (62 mg, 0.1783 mmol) was dissolved in DMF (1 mL) and DIPEA (115 μL, 0.6602 mmol), followed by intermediate XX (43 mg, 0.163 mmol) were added. The mixture was stirred 2 min. before addition of HATU (75 mg, 0.197 mmol) in a single protion. The reaction mixture was stirred at r.t. for 72 h. Water was added and a solid crashed out. The heterogenous mixture was stirred for 1 h at r.t. and then filtered on Buchner. The recovered light yellow solid was dried under vacuum to afford the desired methyl 6-[4-[7-tert-butyl-5-(4-chloro-3-fluoro-phenyl)furo[3,2-b]pyridine-2-carbonyl]-3,3-dimethyl-2-oxo-piperazin-1-yl]pyridine-3-carboxylate (72 mg, 70%). ESI-MS m/z calc. 592.1889, found 594.25 (M+1)+.

### Step 2

Methyl 6-[4-[7-tert-butyl-5-(4-chloro-3-fluoro-phenyl)furo[3,2-b]pyridine-2-carbonyl]-3,3-dimethyl-2-oxo-piperazin-1-yl]pyridine-3-carboxylate (72 mg, 0.1214 mmol) was dissolved in DMSO (5 mL) and aqueous LiOH (242.8 μL of 2 M, 0.4856 mmol) was added. Reaction mixture was stirred 40 minutes and then quenched with HCl 1M (a solid crashed out). Water was added and the solid was stirred for 1 h before being filtered, giving the desired product. The solid was redissolved in 1.25 mL of DMSO and was purified by UV-directed reverse-phase preparative HPLC to afford upon lyophilization the desired 6-[4-[7-tert-butyl-5-(4-chloro-3-fluoro-phenyl)furo[3,2-b]pyridine-2-carbonyl]-3,3-dimethyl-2-oxo-piperazin-1-yl]pyridine-3-carboxylic acid (12.3 mg, 17%). 1H NMR (400 MHz, DMSO-d6) δ 8.98-8.92 (m, 1H), 8.31 (dd, J=8.8, 2.3 Hz, 1H), 8.19 (dd, J=11.0, 2.1 Hz, 1H), 8.12 (d, J=8.6 Hz, 1H), 8.08-8.01 (m, 1H), 7.86 (s, 1H), 7.77-7.68 (m, 1H), 7.66 (s, 1H), 4.35-4.22 (m, 2H), 4.14-3.97 (m, 2H), 1.86 (s, 6H), 1.55 (s, 9H). ESI-MS m/z calc. 578.1732, found 579.4 (M+1)+.

### Example 75: 1-[7-tert-butyl-5-(4-fluorophenyl)furo[3,2-b]pyridine-2-carbonyl]-7,7-dimethyl-1,4-diazepan-5-one

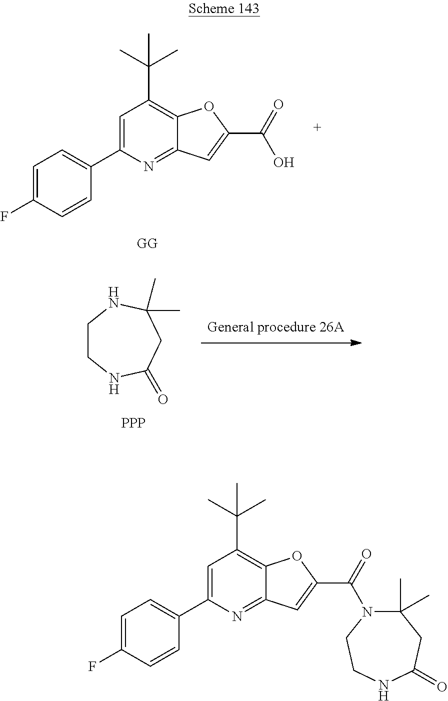

1-[7-tert-butyl-5-(4-fluorophenyl)furo[3,2-b]pyridine-2-carbonyl]-7,7-dimethyl-1,4-diazepan-5-one was prepared using General Procedure 1. Intermediate GG (65 mg, 0.2066 mmol) was dissolved in DMF (1.5 mL) and DIPEA (145 μL, 0.833 mmol), followed by intermediate PPP (104 mg, 0.205 mmol) were added. It was stirred 2 min. before addition of HATU (118 mg, 0.3103 mmol) in a single portion. The reaction mixture was stirred at RT overnight. Water was added along with EtOAc and the phases were separated. The organic phase was washed twice with water and brine (1:1 mixture), dried over MgSO₄, filtered and evaporated under reduced pressure. The residue was purified by SFC (Column: Chiralpak IF SFC Semi-Prep, 250×10 mm, 5 um. Isocratic 40% MeOH during 20 minutes, 15 mL/min flow rate) to afford 1-[7-tert-butyl-5-(4-fluorophenyl)furo[3,2-b]pyridine-2-carbonyl]-7,7-dimethyl-1,4-diazepan-5-one (11.9 mg. 13%). ¹H NMR (400 MHz, Chloroform-d) δ 8.01-7.93 (m, 2H), 7.57 (s, 1H), 7.46 (s, 1H), 7.24-7.13 (m, 2H), 5.68 (s, 1H), 4.06 (t, J=5.8 Hz, 2H), 3.59 (td, J=5.8, 2.9 Hz, 2H), 2.82 (s, 2H), 1.72 (s, 6H), 1.55 (s, 9H). ESI-MS m/z calc. 437.21146, found 438.42 (M+1)⁺.

### Example 76: 4-[7-tert-butyl-5-(4-chlorophenyl)furo[3,2-b]pyridine-2-carbonyl]-3,3-dimethyl-1-(3-methyl-1H-pyrazol-5-yl)piperazin-2-one (I-285)

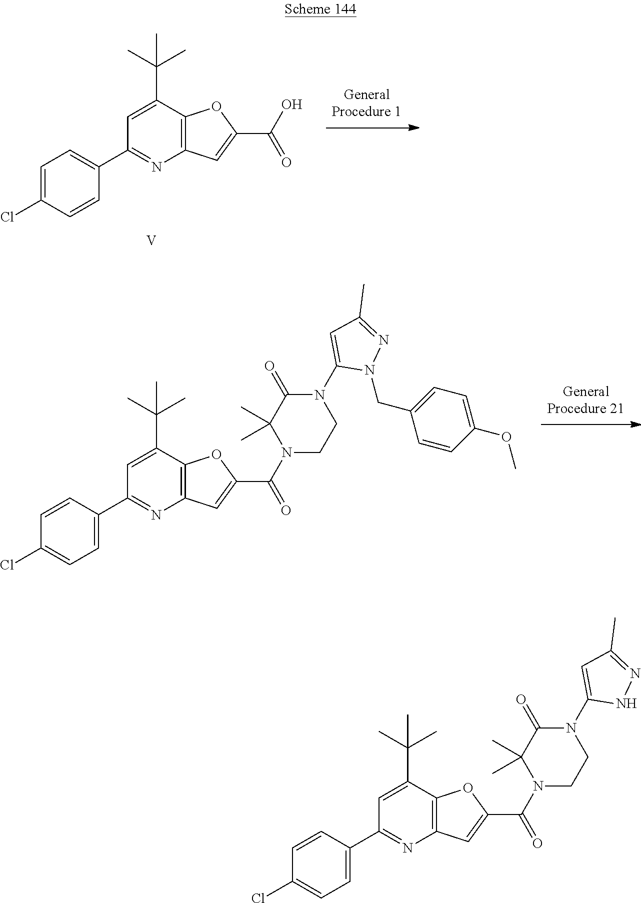

### Step I: 4-[7-tert-butyl-5-(4-chlorophenyl)furo[3,2-b]pyridine-2-carbonyl]-1-[2-[(4-methoxyphenyl)methyl]-5-methyl-pyrazol-3-yl]-3,3-dimethyl-piperazin-2-one

The product was prepared according to General Procedure 1 using 7-tert-butyl-5-(4-chlorophenyl)furo[3,2-b]pyridine-2-carboxylic acid (103 mg, 0.3123 mmol) (Intermediate V). 1-[2-[(4-methoxyphenyl)methyl]-5-methyl-pyrazol-3-yl]-3,3-dimethyl-piperazin-2-one hydrochloride (Intermediate CCC) (144 mg, 0.320 mmol) and HATU (187 mg, 0.492 mmol), and DIPEA (264 μL, 1.516 mmol) in DMF (1.5 mL). After 4.5 h, it was diluted with NH₄Cl sat. (1 mL), H₂O (2 mL) and EtOAc (3 mL). Layers were separated. The aqueous layer was extracted with EtOAc (2×2 mL). The combined organic extracts were washed with saturated NH₄Cl (1 mL), brine (1 mL), dried over Na₂SO₄, filtered and concentrated in a MW vial to obtain 4-[7-tert-butyl-5-(4-chlorophenyl)furo[3,2-b]pyridine-2-carbonyl]-1-[2-[(4-methoxyphenyl)methyl]-5-methyl-pyrazol-3-yl]-3,3-dimethyl-piperazin-2-one (373 mg, 187%).

### Step II: 4-[7-tert-butyl-5-(4-chlorophenyl)furo[3,2-b]pyridine-2-carbonyl]-3,3-dimethyl-1-(3-methyl-1H-pyrazol-5-yl)piperazin-2-one

The intermediate was dissolved in TFA (4 mL) (General Procedure 21). The tube was capped and submitted to microwave for 30 min at 130° C. Then the mixture was concentrated and the residue was purified using prep-HPLC to provide 4-[7-tert-butyl-5-(4-chlorophenyl)furo[3,2-b]pyridine-2-carbonyl]-3,3-dimethyl-1-(3-methyl-1H-pyrazol-5-yl)piperazin-2-one (71 mg, 43%). ¹H NMR (400 MHz, DMSO-d6) δ 12.26 (d, J=1.3 Hz, 1H), 8.16 (d, J=8.6 Hz, 2H), 7.79 (s, 1H), 7.63 (s, 1H), 7.56 (d, J=8.6 Hz, 2H), 6.47 (d, J=1.3 Hz, 1H), 4.15-4.02 (m, 2H), 4.03-3.89 (m, 2H), 2.22 (s, 3H), 1.79 (s, 6H), 1.53 (s, 9H). ESI-MS m/z calc. 519.20374, found 520.65 (M+1)⁺; Retention time: 3.97, using method A.

### Example 77: 4-[7-tert-butyl-5-(4-chloro-3-fluoro-phenyl)furo[3,2-b]pyridine-2-carbonyl]-3,3-dimethyl-1-(3-methyl-1H-pyrazol-5-yl)piperazin-2-one (I-284)

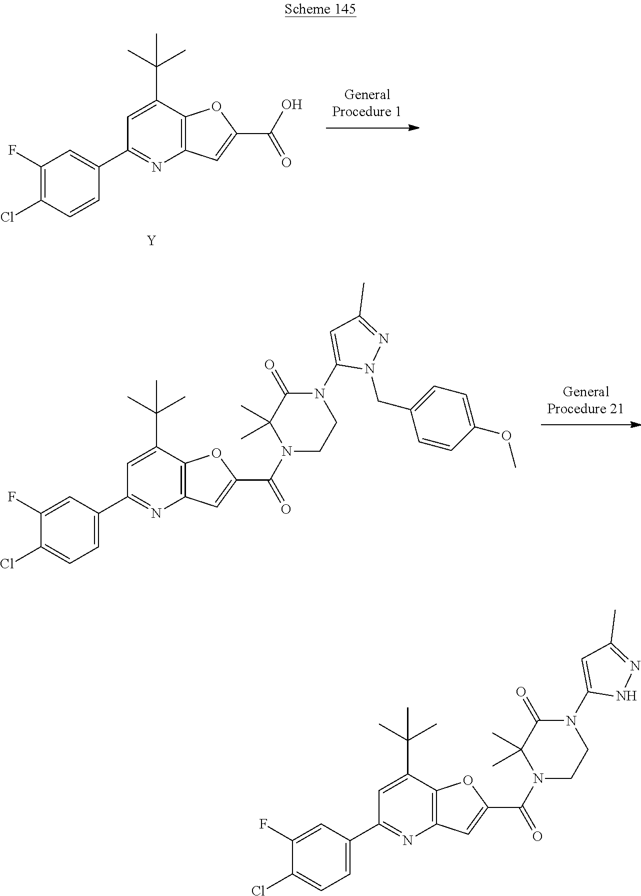

### Step I: 4-[7-tert-butyl-5-(4-chloro-3-fluoro-phenyl)furo[3,2-b]pyridine-2-carbonyl]-1-[2-[(4-methoxyphenyl)methyl]-5-methyl-pyrazol-3-yl]-3,3-dimethyl-piperazin-2-one

The product was prepared according to General Procedure 1. To a vial charged with 7-tert-butyl-5-(4-chloro-3-fluoro-phenyl)furo[3,2-b]pyridine-2-carboxylic acid (106 mg, 0.3048 mmol) (Intermediate V), 1-[2-[(4-methoxyphenyl)methyl]-5-methyl-pyrazol-3-yl]-3,3-dimethyl-piperazin-2-one (Hydrochloric Acid (1)) (144 mg, 0.3197 mmol) (Intermediate CCC) and HATU (Phosphorus Hexafluoride Ion) (179 mg, 0.4708 mmol) was added DMF (1.5 mL) and DIPEA (264 μL, 1.516 mmol). After 4.5 h, the resulting mixture was diluted with NH₄Cl sat. (1 mL), H₂O (2 mL) and EtOAc (3 mL). Layers were separated. Aqueous layer were extracted with EtOAc (2×2 mL). Combined organic extracts were washed with saturated NH₄Cl (1 mL), brine (1 mL), dried over Na₂SO₄, filtered and concentrated to obtain 4-[7-tert-butyl-5-(4-chloro-3-fluoro-phenyl)furo[3,2-b]pyridine-2-carbonyl]-1-[2-[(4-methoxyphenyl)methyl]-5-methyl-pyrazol-3-yl]-3,3-dimethyl-piperazin-2-one (365 mg, 0.5546 mmol).

### Step II: 4-[7-tert-butyl-5-(4-chloro-3-fluorophenyl)furo[3,2-b]pyridine-2-carbonyl]-3,3-dimethyl-1-(3-methyl-1H-pyrazol-5-yl)piperazin-2-one

The intermediate from Step I was dissolved in TFA (4 mL) (General procedure 21). The tube was capped and submitted to microwave for 30 min at 130° C. The mixture was concentrated, and then purified using Prep HPLC to obtain 4-[7-tert-butyl-5-(4-chloro-3-fluoro-phenyl)furo[3,2-b]pyridine-2-carbonyl]-3,3-dimethyl-1-(3-methyl-1H-pyrazol-5-yl)piperazin-2-one (67 mg, 40%). ¹H NMR (400 MHz, DMSO-d6) δ 12.26 (s, 1H), 8.17 (dd, J=11.0, 2.0 Hz, 1H), 8.03 (dd, J=8.5, 1.7 Hz, 1H), 7.84 (s, 1H), 7.71 (t, J=8.2 Hz, 1H), 7.64 (s, 1H), 6.47 (s, 1H), 4.15-4.04 (m, 2H), 4.03-3.89 (m, 2H), 2.22 (s, 3H), 1.79 (s, 6H), 1.53 (s, 9H). ¹⁹F NMR (376 MHz, DMSO-d6) δ −116.04 (dd, J=11.0, 7.9 Hz). ESI-MS m/z calc. 537.1943, found 539.55 (M+1)⁺; Retention time: 4.08 minutes, using Method A).

### Example 78: 4-[7-tert-butyl-5-(4,4-dimethylcyclohexyl)furo[3,2-b]pyridine-2-carbonyl]-3,3-dimethyl-1-(1H-pyrazol-5-yl)piperazin-2-one (I-283)

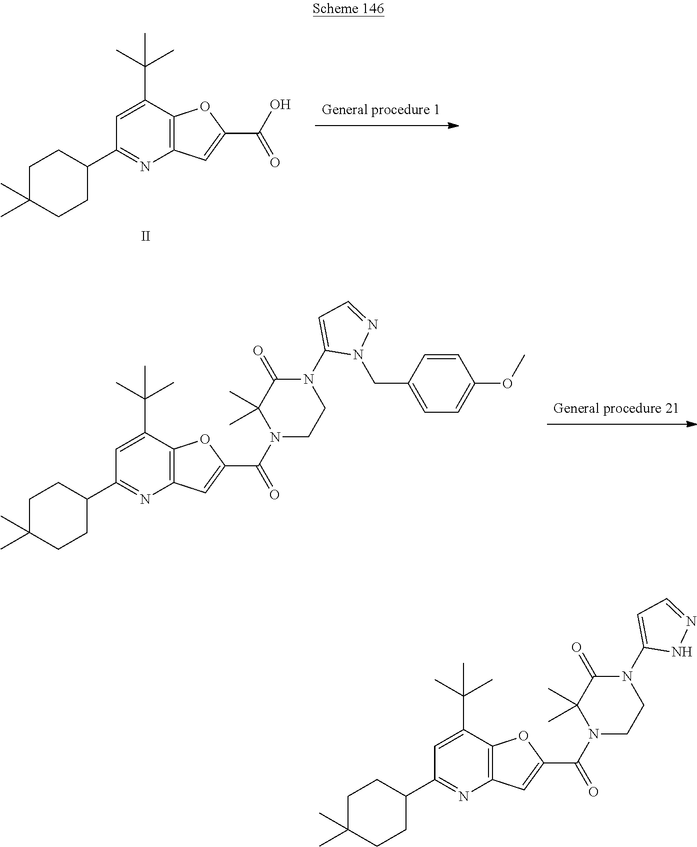

### Step I: 4-[7-tert-butyl-5-(4,4-dimethylcyclohexyl)furo[3,2-b]pyridine-2-carbonyl]-1-[2-[(4-methoxyphenyl)methyl]pyrazol-3-yl]-3,3-dimethyl-piperazin-2-one

According to General procedure 1, to a vial charged with 7-tert-butyl-5-(4,4-dimethylcyclohexyl)furo[3,2-b]pyridine-2-carboxylic acid (105 mg, 0.3187 mmol) (Intermediate II), 1-[2-[(4-methoxyphenyl)methyl]pyrazol-3-yl]-3,3-dimethyl-piperazin-2-one (132 mg, 0.32 mmol) (Intermediate ZZ) and HATU (Phosphorus Hexafluoride Ion) (182 mg, 0.4787 mmol) were added DMF (1.5 mL) and DIPEA (264 μL, 1.516 mmol). After stirring for 4 h, it was diluted with H₂O and EtOAc (2 mL each). The layers were separated. The aqueous layer was extracted with EtOAc (2×2 mL). The combined organic extracts were washed with saturated NH₄Cl (1 mL), brine (1 mL), dried over Na₂SO₄, filtered and concentrated to dryness to obtain 4-[7-tert-butyl-5-(4,4-dimethylcyclohexyl)furo[3,2-b]pyridine-2-carbonyl]-1-[2-[(4-methoxyphenyl)methyl]pyrazol-3-yl]-3,3-dimethyl-piperazin-2-one (408 mg, 0.6520 mmol. 204.6%) is obtained as a brown gum.

### Step II: 4-[7-tert-butyl-5-(4,4-dimethylcyclohexyl)furo[3,2-b]pyridine-2-carbonyl]-3,3-dimethyl-1-(1H-pyrazol-5-yl)piperazin-2-one (I-283)

4-[7-tert-butyl-5-(4,4-dimethylcyclohexyl)furo[3,2-b]pyridine-2-carbonyl]-1-[2-[(4-methoxyphenyl)methyl]pyrazol-3-yl]-3,3-dimethyl-piperazin-2-one was dissolved in TFA (4 mL) in a microwave tube (General procedure 21). The tube was capped and submitted to microwave for 30 min at 130° C. The mixture was concentrated to dryness and the residue was using silica gel chromatography (10 g), eluting with EtOAc in hexanes in a gradient 0% to 80% to provide 4-[7-tert-butyl-5-(4,4-dimethylcyclohexyl)furo[3,2-b]pyridine-2-carbonyl]-3,3-dimethyl-1-(1H-pyrazol-5-yl)piperazin-2-one (76 mg, 45%). ¹H NMR (400 MHz, dmso) δ 12.58 (s, 1H), 7.80-7.63 (m, 1H), 7.52 (s, 1H), 7.17 (s, 1H), 6.70 (t, J=2.2 Hz, 1H), 4.15-4.06 (m, 2H), 4.01-3.87 (m, 2H), 2.70 (tt, J=11.8, 3.9 Hz, 1H), 1.79 (s, 6H), 1.77-1.63 (m, 4H), 1.46 (s+m, 11H), 1.32 (td, J=13.2, 4.1 Hz, 2H), 0.97 (s, 3H), 0.94 (s, 3H). ESI-MS m/z calc. 505.3053, found 507.82 (M+1)⁺; Retention time: 3.97 minutes, Method A.

### Example 79: 6-[4-[7-tert-butyl-5-(4-chlorophenyl)furo[3,2-b]pyridine-2-carbonyl]-3,3-dimethyl-2-oxo-piperazin-1-yl]pyridine-3-carboxylic acid (I-282)

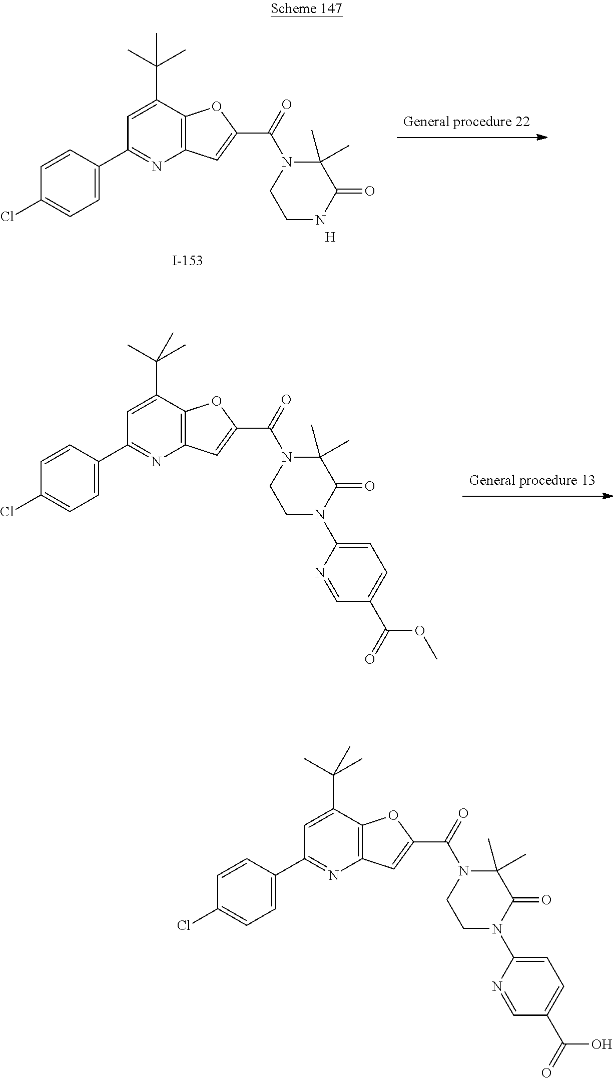

### Step I: methyl 6-[4-[7-tert-butyl-5-(4-chlorophenyl)furo[3,2-b]pyridine-2-carbonyl]-3,3-dimethyl-2-oxo-piperazin-1-yl]pyridine-3-carboxylate

Based on General Procedure 22, in an oven-dry microwave vial under argon were added 4-[7-tert-butyl-5-(4-chlorophenyl)furo[3,2-b]pyridine-2-carbonyl]-3,3-dimethyl-piperazin-2-one (75 mg, 0.159 mmol) (I-153). Pd(OAC)₂ (4 mg, 0.0178 mmol), dioxane (2 mL) and followed by Xantphos (16 mg, 0.0276 mmol), Cs₂CO₃ (72 mg, 0.221 mmol) and methyl 6-bromopyridine-3-carboxylate (38 mg, 0.176 mmol). The heterogenous solution was bubbled with N₂ for 5 minutes before being capped and put in an oil bath at 110° C. for 18 h (ON). Water was added along with EtOAc and the phases were separated. The organic phase was washed twice with water and brine, dried over MgSO₄, filtered and evaporated under reduced pressure. Crude reaction mixture was purified using silica gel chromatography to afford methyl 6-[4-[7-tert-butyl-5-(4-chlorophenyl)furo[3,2-b]pyridine-2-carbonyl]-3,3-dimethyl-2-oxo-piperazin-1-yl]pyridine-3-carboxylate (56 mg. 55%). ¹H NMR (400 MHz, DMSO-d6) δ 8.99 (dd, J=2.4, 0.8 Hz, 1H), 8.35 (dd, J=8.8, 2.4 Hz, 1H), 8.21-8.10 (m, 3H), 7.80 (s, 1H), 7.65 (s, 1H), 7.61-7.52 (m, 2H), 4.36-4.27 (m, 2H), 4.12-4.06 (m, 2H), 3.89 (s, 3H), 1.87 (s, 6H), 1.54 (s, 9H). ESI-MS m/z calc. 574.1983, found 576.35 (M+1)⁺.

### Step II: 6-[4-[7-tert-butyl-5-(4-chlorophenyl)furo[3,2-b]pyridine-2-carbonyl]-3,3-dimethyl-2-oxo-piperazin-1-yl]pyridine-3-carboxylic acid (I-282)

Based on General Procedure 13, methyl 6-[4-[7-tert-butyl-5-(4-chlorophenyl)furo[3,2-b]pyridine-2-carbonyl]-3,3-dimethyl-2-oxo-piperazin-1-yl]pyridine-3-carboxylate (56 mg, 0.0974 mmol) was dissolved in Dioxane (2.5 mL) and then 1M NaOH (300 μL of 1 M. 0.300 mmol) was added. The reaction mixture was stirred at rt for 18 h. 1M HCl was added until acidic pH was reached. Water was added along with EtOAc and the phases were separated. The organic phase was washed twice with water and brine, dried over MgSO₄, filtered and evaporated under reduced pressure. The crude reaction mixture was purified UV directed reverse phase HPLC to afford 6-[4-[7-tert-butyl-5-(4-chlorophenyl)furo[3,2-b]pyridine-2-carbonyl]-3,3-dimethyl-2-oxo-piperazin-1-yl]pyridine-3-carboxylic acid (23 mg, 42%). ¹H NMR (400 MHz, DMSO-d6) δ 8.96 (dd, J=2.3, 0.8 Hz, 1H), 8.32 (dd, J=8.8, 2.4 Hz, 1H), 8.21-8.12 (m, 2H), 8.12 (dd, J=8.8, 0.8 Hz, 1H), 7.80 (s, 1H), 7.65 (s, 1H), 7.61-7.52 (m, 2H), 4.33-4.26 (m, 2H), 4.13-4.03 (m, 2H), 1.86 (s, 6H), 1.54 (s, 9H). ESI-MS m/z calc. 560.1827, found 562.63 (M+1)⁺; Retention time: 4.23 min, Method A.

### Example 80: 6-[4-[7-tert-butyl-5-(4,4-dimethylcyclohexyl)furo[3,2-b]pyridine-2-carbonyl]-3,3-dimethyl-2-oxo-piperazin-1-yl]pyridine-3-carboxylic acid (I-281)

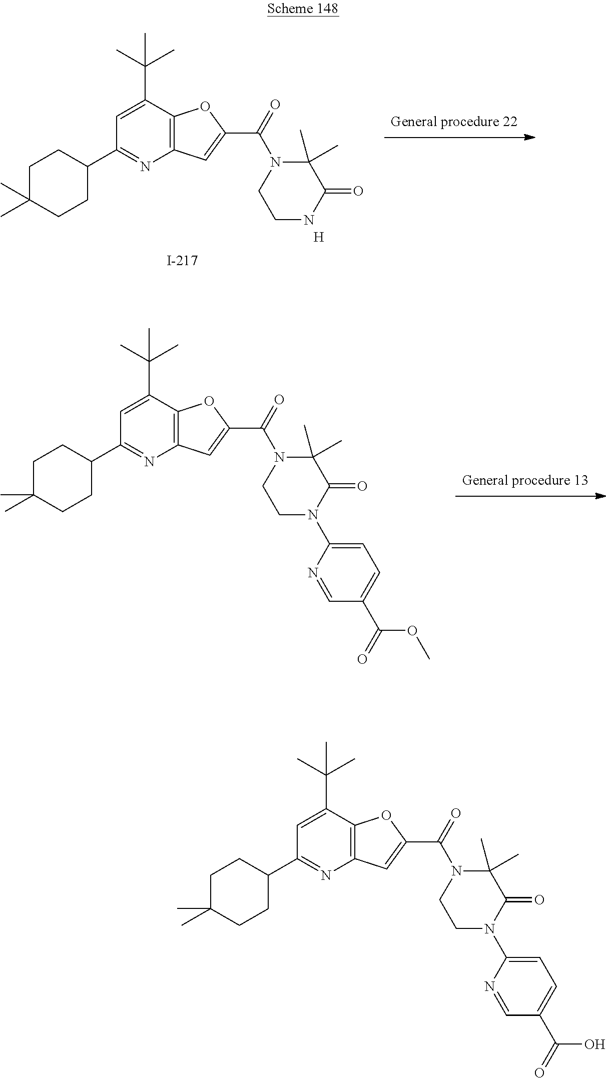

### Step I: methyl 6-[4-[7-tert-butyl-5-(4,4-dimethylcyclohexyl)furo[3,2-b]pyridine-2-carbonyl]-3,3-dimethyl-2-oxo-piperazin-1-yl]pyridine-3-carboxylate

Using General Procedure 22, in an oven-dry microwave vial under argon was dissolved 4-[7-tert-butyl-5-(4,4-dimethylcyclohexyl)furo[3,2-b]pyridine-2-carbonyl]-3,3-dimethyl-piperazin-2-one (100 mg, 0.209 mmol) (I-217) in Dioxane (1.5 mL) and Pd(OAC)₂ (6 mg, 0.0267 mmol) followed by Xantphos (22 mg, 0.0380 mmol), Cs₂CO₃ (95 mg, 0.292 mmol) and methyl 6-bromopyridine-3-carboxylate (54 mg, 0.250 mmol) were added. The heterogeneous solution was bubbled with N₂ for 5 minutes before being capped and put in an oil bath at 110° C. for 18 h (ON). Water was added along with EtOAc and the phases were separated. The organic phase was washed twice with water and brine (1:1 mixture), dried over MgSO₄, filtered and evaporated under reduced pressure. Crude reaction mixture was purified by silic gel chromatography to afford methyl 6-[4-[7-tert-butyl-5-(4,4-dimethylcyclohexyl)furo[3,2-b]pyridine-2-carbonyl]-3,3-dimethyl-2-oxo-piperazin-1-yl]pyridine-3-carboxylate (57 mg, 48%). ¹H NMR (400 MHz, DMSO-d6) δ 8.98 (dd, J=2.4, 0.8 Hz, 1H), 8.35 (dd, J=8.8, 2.4 Hz, 1H), 8.16 (dd, J=8.8, 0.8 Hz, 1H), 7.54 (s, 1H), 7.18 (s, 1H), 4.31-4.25 (m, 2H), 4.09-4.01 (m, 2H), 3.89 (s, 3H), 1.85 (s, 6H), 1.82-1.64 (m, 4H), 1.47 (s, 12H), 1.40-1.28 (m, 2H), 0.97 (d, J=9.4 Hz, 6H). ESI-MS m/z calc. 574.3155, found 575.3 (M+1)⁺;

### Step II: 6-[4-[7-tert-butyl-5-(4,4-dimethylcyclohexyl)furo[3,2-b]pyridine-2-carbonyl]-3,3-dimethyl-2-oxo-piperazin-1-yl]pyridine-3-carboxylic acid (I-281)

Based on General Procedure 13, methyl 6-[4-[7-tert-butyl-5-(4,4-dimethylcyclohexyl)furo[3,2-b]pyridine-2-carbonyl]-3,3-dimethyl-2-oxo-piperazin-1-yl]pyridine-3-carboxylate (57 mg, 0.0992 mmol) was dissolved in Dioxane (2.5 mL) and 1M NaOH (300 μL of 1 M, 0.300 mmol) was added. The reaction mixture was stirred at rt for 18 h. Then 1 M HCl was added until acidic pH was reached. Water was added along with EtOAc and the phases were separated. The organic phase was twice with water and brine, dried over MgSO₄, filtered and evaporated under reduced pressure. The crude mixture was dissolved in 1 mL of DMF, filtered purified using mass directed reverse phase prep-HPLC to afford 6-[4-[7-tert-butyl-5-(4,4-dimethylcyclohexyl)furo[3,2-b]pyridine-2-carbonyl]-3,3-dimethyl-2-oxo-piperazin-1-yl]pyridine-3-carboxylic acid (6.5 mg, 11%). ¹H NMR (400 MHz, DMSO-d6) δ 8.97-8.92 (m, 1H), 8.31 (dd, J=8.8, 2.3 Hz, 1H), 8.10 (d, J=8.8 Hz, 1H), 7.53 (s, 1H), 7.18 (s, 1H), 4.32-4.22 (m, 2H), 4.09-3.98 (m, 2H), 2.79-2.63 (m, 1H), 1.90-1.63 (m, 10H), 1.47 (s, 11H), 1.41-1.27 (m, 2H), 0.97 (d, J=9.6 Hz, 6H). ESI-MS m/z calc. 560.29987, found 561.93 (M+1)⁺; Retention time: 4.39 minutes, Method A.

### Example 81

### 5-[4-[7-tert-butyl-5-(4-chloro-3-fluoro-phenyl)furo[3,2-b]pyridine-2-carbonyl]-3,3-dimethyl-2-oxo-piperazin-1-yl]-1H-pyrazole-3-carboxylic acid (I-280)

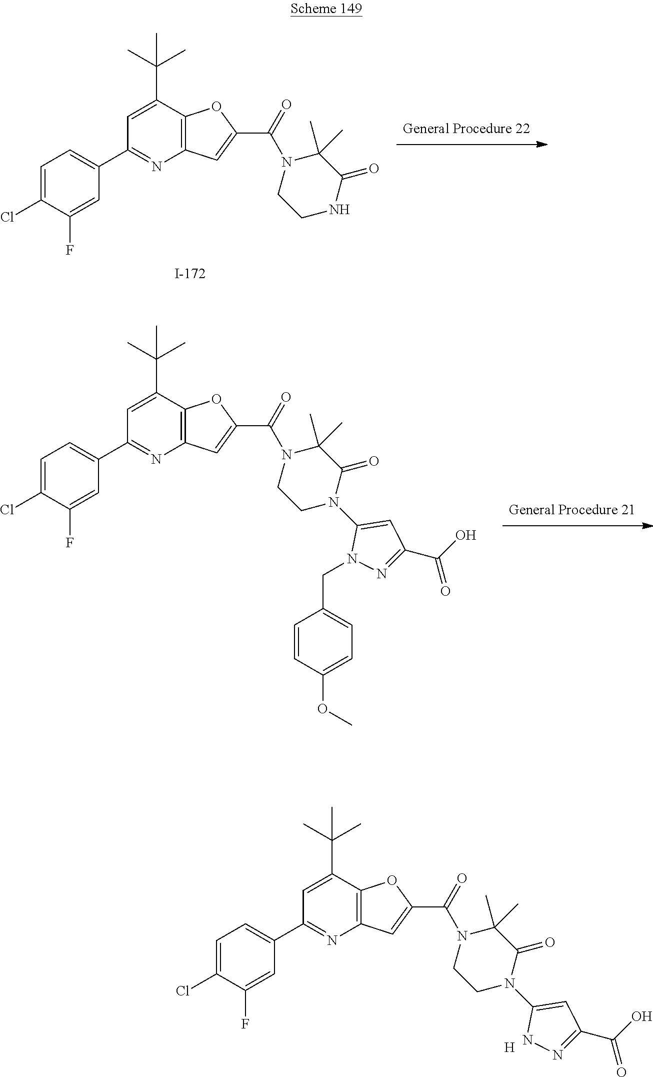

### Step I: 4-[4-[7-tert-butyl-5-(4-chloro-3-fluoro-phenyl)furo[3,2-b]pyridine-2-carbonyl]-3,3-dimethyl-2-oxo-piperazin-1-yl]-1-[(4-methoxyphenyl)methyl]pyrazole-3-carboxylic acid

According to General Procedure 22, a mixture of 4-[7-tert-butyl-5-(4-chloro-3-fluoro-phenyl)furo[3,2-b]pyridine-2-carbonyl]-3,3-dimethyl-piperazin-2-one (251 mg, 0.510 mmol) (Intermediate JJJ), methyl 5-iodo-1-[(4-methoxyphenyl)methyl]pyrazole-3-carboxylate (252 mg, 0.6771 mmol) (I-172), CuI (62 mg, 0.326 mmol), K₃PO₄ (232 mg. 1.09 mmol) was charged in a microwave vial. Then to it was added DMF (2.5 mL) and N,N′-dimethylethane-1,2-diamine (66 μL, 0.602 mmol). It was degassed again and transferred to a preheated (120° C.) oil bath and stirred overnight. The reaction was cooled down to rt, it was then diluted with EtOAc (5 mL) and 1 N HCl (3 mL). The Layers were separated. Then the aqueous layer was extracted with EtOAc (2×2 mL). The combined organic extracts were washed with brine (2 mL), dried over Na₂SO₄, filtered and concentrated.

### Step III: 5-[4-[7-tert-butyl-5-(4-chloro-3-fluoro-3-fluoro-phenyl)furo[3,2-b]pyridine-2-carbonyl]-3,3-dimethyl-2-oxo-piperazin-1-yl]-1H-pyrazole-3-carboxylic acid (I-280)

The crude residue from Step II was transferred to a microwave vial using TFA (4.0 mL). The tube was capped and submitted to microwave for 30 min at 130° C. (General procedure 21). The volatiles were removed under reduced pressure. The residue was purified by UV directed reverse phase prep-HPLC to provide 5-[4-[7-tert-butyl-5-(4-chloro-3-fluoro-phenyl)furo[3,2-b]pyridine-2-carbonyl]-3,3-dimethyl-2-oxo-piperazin-1-yl]-1H-pyrazole-3-carboxylic acid (95 mg, 31%). ¹H NMR (400 MHz, DMSO-d6) δ 13.64 (broad s, 1H), 13.49 (broad s, 1H), 8.18 (dd, J=11.0, 2.0 Hz, 1H), 8.04 (dd, J=8.4, 1.6 Hz, 1H), 7.85 (s, 1H), 7.71 (t, J=8.2 Hz, 1H), 7.65 (s, 1H), 7.15 (s, 1H), 4.21-4.08 (m, 2H), 4.07-3.95 (m, 2H), 1.81 (s, 6H), 1.54 (s, 9H), 9F NMR (376 MHz, DMSO-d6) δ −116.04 (dd, J=10.9, 7.9 Hz). 0.47% wt TFA. ESI-MS m/z calc. 567.16846, found 569.53 (M+1)⁺; Retention time: 3.92 minutes, Method A.

### Example 82: 5-[4-[7-tert-butyl-5-(4-chlorophenyl)furo[3,2-b]pyridine-2-carbonyl]-3,3-dimethyl-2-oxo-piperazin-1-yl]-1H-pyrazole-3-carboxylic acid (I-278)

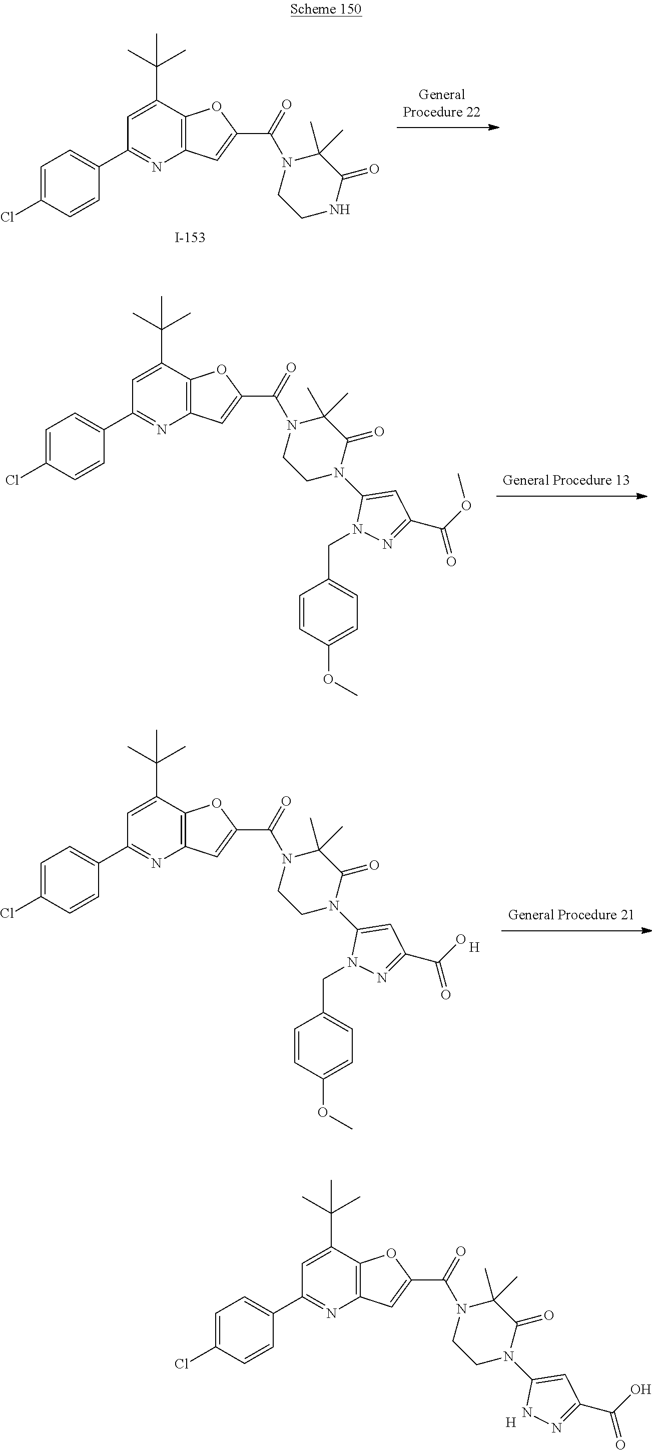

### Step I: methyl 5-[4-[7-tert-butyl-5-(4-chlorophenyl)furo[3,2-b]pyridine-2-carbonyl]-3,3-dimethyl-2-oxo-piperazin-1-yl]-1-[(4-methoxyphenyl)methyl]pyrazole-3-carboxylate

Based on General Procedure 22, a mixture of 4-[7-tert-butyl-5-(4-chlorophenyl)furo[3,2-b]pyridine-2-carbonyl]-3,3-dimethyl-piperazin-2-one (247 mg, 0.522 mmol) (I-153), methyl 5-iodo-1-[(4-methoxyphenyl)methyl]pyrazole-3-carboxylate (248 mg, 0.666 mmol) (Intermediate JJJ), CuI (49.71 mg, 0.261 mmol), K₃PO₄ (225 mg, 1.06 mmol) was charged in a microwave vial. It was degassed (house-vac then N₂, 3×). Then DMF (2.5 mL) and N,N′-dimethylethane-1,2-diamine (56 μL, 0.526 mmol) were added. It was dgassed again and transferred to a preheated (120° C.) oil bath. After 5 h, it was cooled down to room temperature, diluted with EtOAc (5 mL) and 1 N HCl (3 mL). The layers were separated and the aqueous layer was extracted with EtOAc (2×2 mL). The combined organic extracts were washed with brine (2 mL), dried over Na₂SO₄, filtered and concentrated to obtain 620 mg of the desired product.

### Step II: 5-[4-[7-tert-butyl-5-(4-chlorophenyl)furo[3,2-b]pyridine-2-carbonyl]-3,3-dimethyl-2-oxo-piperazin-1-yl]-1-[(4-methoxyphenyl)methyl]pyrazole-3-carboxylic acid

The mixture from Step I was hydrolyzed based General Procedure 13. The crude mixture was further purified on mass directed reverse phase prep-HPLC to provide 5-[4-[7-tert-butyl-5-(4-chlorophenyl)furo[3,2-b]pyridine-2-carbonyl]-3,3-dimethyl-2-oxo-piperazin-1-yl]-1-[(4-methoxyphenyl)methyl]pyrazole-3-carboxylic acid (250 mg, 71%). ESI-MS m/z calc. 669.2354, found 670.13 (M+1)⁺.

### Step III: 5-[4-[7-tert-butyl-5-(4-chlorophenyl)furo[3,2-b]pyridine-2-carbonyl]-3,3-dimethyl-2-oxo-piperazin-1-yl]-1H-pyrazole-3-carboxylic acid (I-279)

5-[4-[7-tert-butyl-5-(4-chlorophenyl)furo[3,2-b]pyridine-2-carbonyl]-3,3-dimethyl-2-oxo-piperazin-1-yl]-1-[(4-methoxyphenyl)methyl]pyrazole-3-carboxylic acid (250 mg, 0.373 mmol) was deprotected using General procedure 21 in microwave reactor. The mixture was purified by mass directed reverse phase prep-HPLC to provide 5-[4-[7-tert-butyl-5-(4-chlorophenyl)furo[3,2-b]pyridine-2-carbonyl]-3,3-dimethyl-2-oxo-piperazin-1-yl]-1H-pyrazole-3-carboxylic acid (14 mg, 5%). ¹H NMR (400 MHz, DMSO-d6) δ 12.90 (broad s, 1H), 8.28-8.09 (m, 2H), 7.79 (s, 1H), 7.63 (s, 1H), 7.60-7.46 (m, 2H), 6.88 (s, 1H), 4.19-4.07 (m, 2H), 4.06-3.90 (m, 2H), 1.80 (s, 6H), 1.53 (s, 9H). ESI-MS m/z calc. 549.1779, found 551.52 (M+1)⁺; Retention time: 3.8 minutes, Method A.

### Example 83: 4-[7-tert-butyl-5-(4-chloro-3-fluoro-phenyl)furo[3,2-b]pyridine-2-carbonyl]-3,3-dimethyl-1-pyrimidin-2-yl-piperazin-2-one

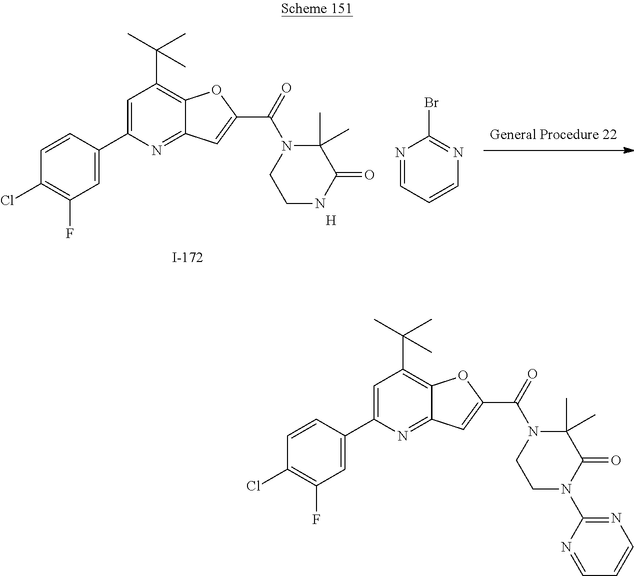

### 4-[7-tert-butyl-5-(4-chloro-3-fluoro-phenyl)furo[3,2-b]pyridine-2-carbonyl]-3,3-dimethyl-1-pyrimidin-2-yl-piperazin-2-one (I-172)

According to General procedure 22, 4-[7-tert-butyl-5-(4-chloro-3-fluoro-phenyl)furo[3,2-b]pyridine-2-carbonyl]-3,3-dimethyl-piperazin-2-one (50 mg) (I-172), 2-bromopyrimidine, Pd(OAc)₂, Xantphos and Cs₂CO₃ were charged in a 4 mL vial. The mixture was flushed with N₂ and anhydrous dioxane (330 μL) was added via syringe. The reaction was stirred at 100° C. for overnight. The mixture was cooled down to rt and dissolved in DCM, followed by addition of SilisMetS Thiol powder as the heavy metal scavenger. The mixture was stirred for 2 hours and filtered through a celite pad, washed with DCM and concentrated. The residue was purified using reverse phase Prep HPLC to give 4-[7-tert-butyl-5-(4-chloro-3-fluoro-phenyl)furo[3,2-b]pyridine-2-carbonyl]-3,3-dimethyl-piperazin-2-one (50 mg, 0.109 mmol). ¹H NMR (400 MHz, DMSO-d6) δ 8.88-8.82 (m, 2H), 8.24-8.16 (m, 1H), 8.11-8.00 (m, 1H), 7.86 (s, 1H), 7.79-7.68 (m, 1H), 7.67 (s, 1H), 7.48-7.37 (m, 1H), 4.25-4.17 (m, 2H), 4.10-4.02 (m, 2H), 1.83 (s, 6H), 1.53 (s, 9H). ESI-MS m/z calc. 535.17865, found 537.33 (M+1)⁺; Retention time: 4.15 minutes, Method A.

### Example 84: 4-[7-tert-butyl-5-(4-chloro-3-methyl-phenyl)furo[3,2-b]pyridine-2-carbonyl]-3,3-dimethyl-1-(1H-pyrazol-4-yl)piperazin-2-one (I-275)

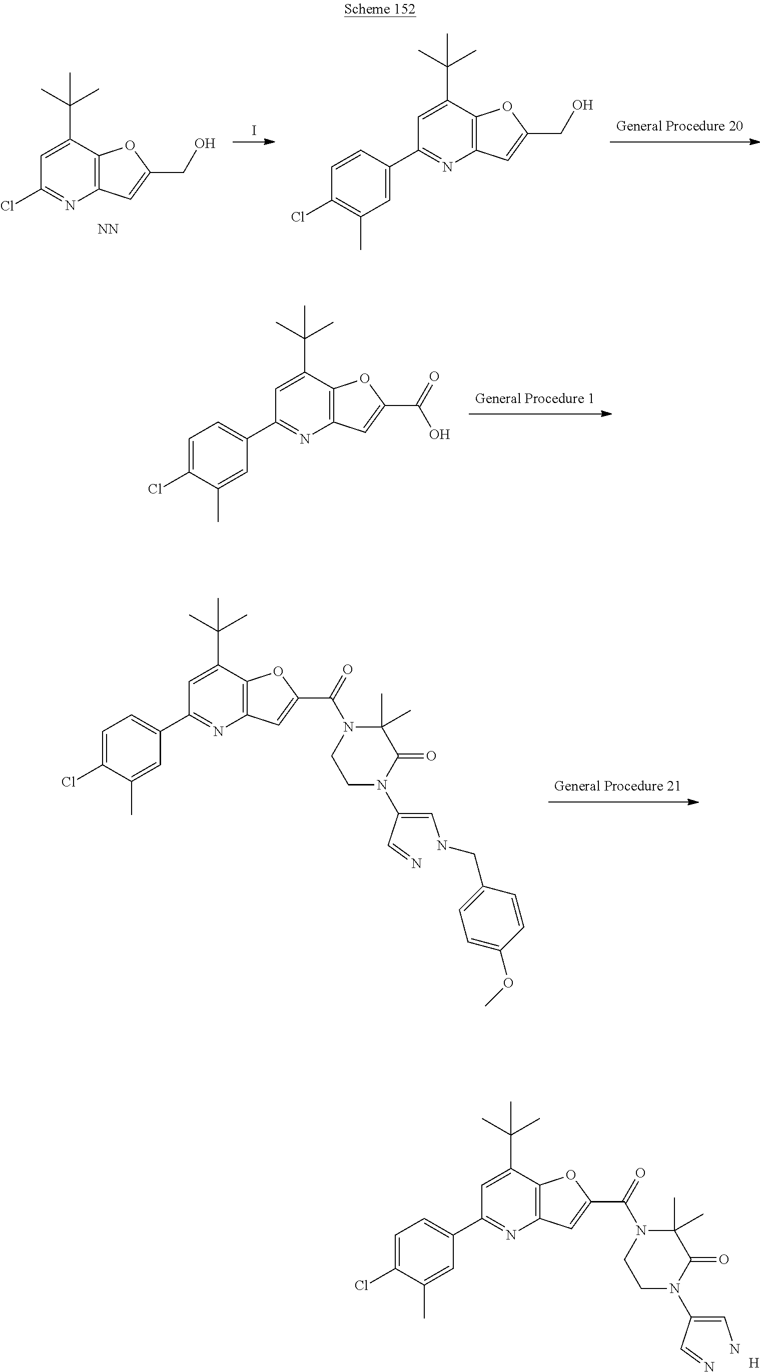

### Step I: [7-tert-butyl-5-(4-chloro-3-methyl-phenyl)furo[3,2-b]pyridin-2-yl]methanol

In a pressured vessel were charged with (7-tert-butyl-5-chloro-furo[3,2-b]pyridin-2-yl)methanol (2 g, 8.34 mmol) (Intermediate NN), (4-chloro-3-methyl-phenyl)boronic acid (1.6 g, 9.39 mmol) and Pd(PPh₃)₄ (499 mg, 0.432 mmol). It was flushed with N₂ for 10 min, and to it was added 1,4-dioxane (30 mL) and 2M Na₂CO₃ aqueous (10.5 mL of 2 M, 21.00 mmol). It was then degassed and heated at 80° C. overnight (20 h) under nitrogen. After cooling down to room temperature, it was then diluted with EtOAc (100 mL) and H₂O (50 mL). The layers were separated. The aqueous layer was extracted with EtOAc (2×50 mL). The combined organic extracts were washed with brine (50 mL), dried over MgSO₄, filtered over a 10 g silica cartridge and concentrated. The residue was purified by silica gel chromatography to obtain [7-tert-butyl-5-(4-chloro-3-methyl-phenyl)furo[3,2-b]pyridin-2-yl]methanol (2.68 g, 97%). ¹H NMR (400 MHz, DMSO-d6) δ 8.08 (d, J=1.8 Hz, 1H), 7.92 (dd, J=8.4, 2.1 Hz, 1H), 7.61 (s, 1H), 7.49 (d, J=8.4 Hz, 1H), 6.92 (s, 1H), 5.63 (t, J=5.9 Hz, 1H), 4.65 (d, J=5.9 Hz, 2H), 2.42 (s, 3H), 1.50 (s, 9H). ESI-MS m/z calc. 329.11826, found 329.52 (M+1)⁺; Retention time: 0.97 minutes.

### Step II: 7-tert-butyl-5-(4-chloro-3-methyl-phenyl)furo[3,2-b]pyridine-2-carboxylic acid

The title compound was prepared based on General Procedure 20 using [7-tert-butyl-5-(4-chloro-3-methyl-phenyl)furo[3,2-b]pyridin-2-yl]methanol (2.68 g, 8.126 mmol), NMO (8.81 g. 75.21 mmol) and TPAP (142.8 mg, 0.4063 mmol). MeCN (10 mL) and IPA (2 mL) to provide 7-tert-butyl-5-(4-chloro-3-methyl-phenyl)furo[3,2-b]pyridine-2-carboxylic acid (2.22 g, 6.457 mmol. 79.46%). ¹H NMR (400 MHz, DMSO-d6) δ 13.90 (s, 1H), 8.12 (d, J=1.8 Hz, 1H), 7.96 (dd, J=8.0, 2.3 Hz, 1H), 7.81 (s, 1H), 7.79 (s, 1H), 7.53 (d, J=8.4 Hz, 1H), 2.43 (s, 3H), 1.53 (s, 9H). ESI-MS m/z calc. 343.09753, found 346.42 (M+1)⁺; Retention time: 0.99 minutes.

### Step III: 4-[7-tert-butyl-5-(4-chloro-3-methyl-phenyl)furo[3,2-b]pyridine-2-carbonyl]-1-[1-[(4-methoxyphenyl)methyl]pyrazol-4-yl]-3,3-dimethyl-piperazin-2-one

The title compound was prepared according to General Procedure 1, using 7-tert-butyl-5-(4-chloro-3-methyl-phenyl)furo[3,2-b]pyridine-2-carboxylic acid (251 mg, 0.730 mmol), 1-[1-[(4-methoxyphenyl)methyl]pyrazol-4-yl]-3,3-dimethyl-piperazin-2-one (Hydrochloric Acid) (264 mg, 0.745 mmol) (Intermediate JJJ), DIPEA (640 μL, 3.67 mmol) and HATU (359 mg, 0.944 mmol) in DMF (3.8 mL) to provide 4-[7-tert-butyl-5-(4-chloro-3-methyl-phenyl)furo[3,2-b]pyridine-2-carbonyl]-1-[1-[(4-methoxyphenyl)methyl]pyrazol-4-yl]-3,3-dimethyl-piperazin-2-one was obtained.

### Step IV: 4-[7-tert-butyl-5-(4-chloro-3-methyl-phenyl)furo[3,2-b]pyridine-2-carbonyl]-3,3-dimethyl-1-(1H-pyrazol-4-yl)piperazin-2-one (I-275)

The title compound was prepared based on General Procedure 21, using the intermediate from Step III and TFA (4 mL). After workup, the residue was purified using reverse phase prep-HPLC C18 column eluting with CAN/water to obtain an off-white solid. It was then crystalized in EtOAc to obtain 4-[7-tert-butyl-5-(4-chloro-3-methyl-phenyl)furo[3,2-b]pyridine-2-carbonyl]-3,3-dimethyl-1-(1H-pyrazol-4-yl)piperazin-2-one (127 mg, 33%). ¹H NMR (400 MHz, DMSO-d6) δ 12.82 (broad s, 1H), 8.12 (d, J=2.0 Hz, 1H), 8.05 (d, J=1.1 Hz, 1H), 7.97 (dd, J=8.4, 2.0 Hz, 1H), 7.79 (s, 1H), 7.75 (d, J=1.8 Hz, 1H), 7.63 (s, 1H), 7.53 (d, J=8.4 Hz, 1H), 4.06-3.97 (m, 2H), 3.98-3.88 (m, 2H), 2.44 (s, 3H), 1.79 (s, 6H), 1.54 (s, 9H). ESI-MS m/z calc. 519.20374, found 521.5 (M+1)⁺; Retention time: 4.03 minutes, Method A.

### Example 85: 7-tert-butyl-5-(4-chloro-3-fluoro-phenyl)furo[3,2-b]pyridin-2-yl]-[2,2-dimethyl-4-(2-pyridyl)piperazin-1-yl]methanone (I-271)

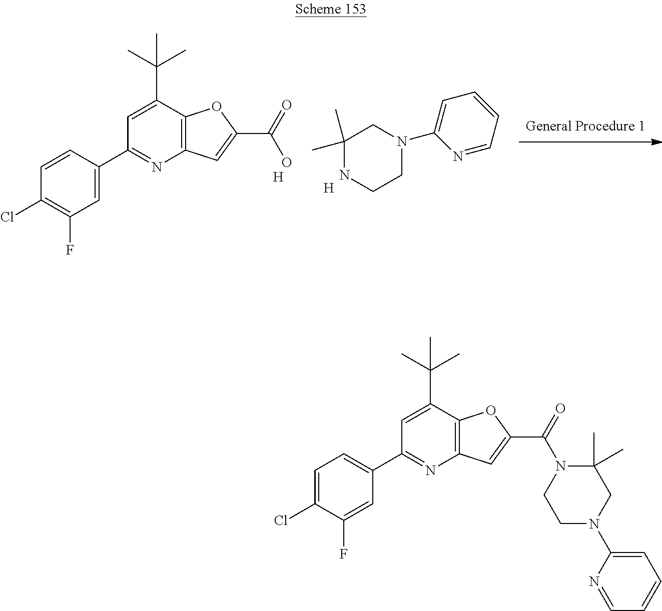

The title compound was prepared according to General Procedure 1, 7-tert-butyl-5-(4-chloro-3-fluoro-phenyl)furo[3,2-b]pyridine-2-carboxylic acid (50 mg, 0.144 mmol) (Intermediate Y), 3,3-dimethyl-1-(2-pyridyl)piperazine (Hydrochloric Acid (1)) (37 mg, 0.163 mmol) (Intermediate MMM) and HATU (Phosphorus Hexafluoride Ion) (84 mg, 0.221 mmol) in DMF (750 μL) and DIPEA (100 μL, 0.574 mmol). The product 7-tert-butyl-5-(4-chloro-3-fluoro-phenyl)furo[3,2-b]pyridin-2-yl]-[2,2-dimethyl-4-(2-pyridyl)piperazin-1-yl]methanone (53 mg, 69%) was obtained. ¹H NMR (400 MHz, DMSO-d6) δ 8.17 (dd, J=11.0, 2.1 Hz, 1H), 8.09 (ddd, J=4.9, 2.0, 0.8 Hz, 1H), 8.07-7.95 (m, 1H), 7.83 (s, 1H), 7.78-7.64 (m, 1H), 7.61-7.50 (m, 2H), 6.66-6.51 (m, 2H), 4.15-4.01 (m, 2H), 3.90 (s, 2H), 3.58 (t, J=5.6 Hz, 2H), 1.53 (s, 10H), 1.51 (s, 6H). ¹⁹F NMR (376 MHz, DMSO-d6) δ −116.05 (dd, J=11.0, 7.9 Hz). ESI-MS m/z calc. 520.20416, found 523.27 (M+1)⁺; Retention time: 3.45 minutes, Method A.

### Example 86: [7-tert-butyl-5-(4-chloro-3-fluoro-phenyl)furo[3,2-b]pyridin-2-yl]-[2,2-dimethyl-4-(3-pyridyl)piperazin-1-yl]methanone (I-267)

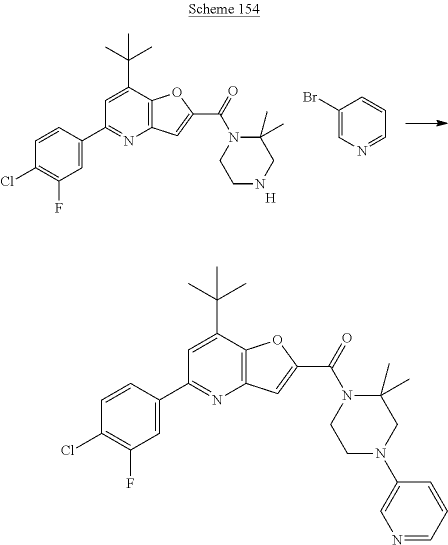

The title compound was prepared according to General Procedure 22, using [7-tert-butyl-5-(4-chloro-3-fluoro-phenyl)furo[3,2-b]pyridin-2-yl]-(2,2-dimethylpiperazin-1-yl)methanone (Hydrochloric Acid (2)) (74 mg, 0.137 mmol) (Intermediate KK), rac-BINAP (11.4 mg, 0.0183 mmol), Pd₂(dba)₃ (7.2 mg, 0.00797 mmol) and NaOtBu (42 mg, 0.437 mmol) in toluene (750 μL). The compound [7-tert-butyl-5-(4-chloro-3-fluoro-phenyl)furo[3,2-b]pyridin-2-yl]-[2,2-dimethyl-4-(3-pyridyl)piperazin-1-yl]methanone (56 mg, 77%) was obtained. ¹H NMR (400 MHz, DMSO-d6) δ 8.17 (dd+broad s, J=11.0, 2.0 Hz, 2H), 8.03 (dd, J=8.4, 1.7 Hz, 1H), 7.89 (broad s, 1H), 7.83 (s, 1H), 7.76-7.64 (m, 1H), 7.55 (s, 1H), 7.26-7.05 (m, 2H), 4.05 (t, J=5.4 Hz, 2H), 3.61 (s, 2H), 3.49 (t, J=5.4 Hz, 2H), 1.53 (s, 15H). ¹⁹F NMR (376 MHz, DMSO-d6) δ −116.05 (dd, J=11.0, 7.9 Hz). ESI-MS nm/z calc. 520.20416, found 522.39 (M+1)⁺; Retention time: 3.33 minutes, Method A.

### Example 87: (3S,5S,6S)-6-[6-[4-[7-tert-butyl-5-(4-chloro-3-fluoro-phenyl)furo[3,2-b]pyridine-2-carbonyl]-3,3-dimethyl-piperazin-1-yl]pyridine-2-carbonyl]oxy-3,4,5-trihydroxy-tetrahydropyran-2-carboxylic acid (I-253)

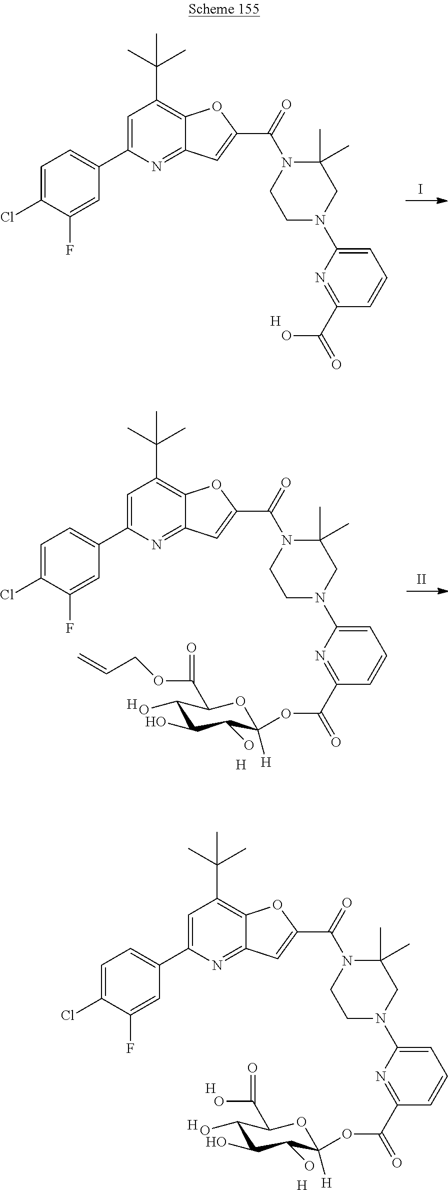

### Step I: [(2S,3S,5S)-6-allyloxycarbonyl-3,4,5-trihydroxy-tetrahydropyran-2-yl]6-[4-[7-tert-butyl-5-(4-chloro-3-fluoro-phenyl)furo[3,2-b]pyridine-2-carbonyl]-3,3-dimethyl-piperazin-1-yl]pyridine-2-carboxylate

To 6-[4-[7-tert-butyl-5-(4-chloro-3-fluoro-phenyl)furo[3,2-b]pyridine-2-carbonyl]-3,3-dimethyl-piperazin-1-yl]pyridine-2-carboxylic acid (108.9 mg, 0.1927 mmol) (I-448) in DCM (3 mL) was added oxalyl dichloride (115 μL of 2 M, 0.231 mmol) and refluxed 2 h. The reaction mixture was azetroped with toluene and re-suspended in DCM. To this was added allyl (3S,5S,6R)-3,4,5,6-tetrahydroxytetrahydropyran-2-carboxylate (90.26 mg, 0.3854 mmol) and TEA (39.00 mg, 54 μL, 0.385 mmol). The reaction mixture was stirred at rt overnight. The reaction mixture was used directly in the next step without purification.

### Step II: (3S,5S,6S)-6-[6-[4-[7-tert-butyl-5-(4-chloro-3-fluoro-phenyl)furo[3,2-b]pyridine-2-carbonyl]-3,3-dimethyl-piperazin-1-yl]pyridine-2-carbonyl]oxy-3,4,5-trihydroxy-tetrahydropyran-2-carboxylic acid

To the mixture in Step I in THF (3 mL) was added palladium;triphenylphosphane (22.2 mg, 0.0193 mmol) and pyrrolidine (13.7 mg, 16.09 μL, 0.193 mmol) and stirred at rt for 1 h. The reaction mixture was filtered and purified by reverse phase prep HPLC to give (3S,5S,6S)-6-[6-[4-[7-tert-butyl-5-(4-chloro-3-fluoro-phenyl)furo[3,2-b]pyridine-2-carbonyl]-3,3-dimethyl-piperazin-1-yl]pyridine-2-carbonyl]oxy-3,4,5-trihydroxy-tetrahydropyran-2-carboxylic acid (20 mg). ¹H NMR (400 MHz, DMSO-d6) δ 8.18 (dd, J=11.0, 2.1 Hz, 1H), 8.05 (dd, J=8.5, 2.0 Hz, 1H), 7.84 (s, 1H), 7.74 (dt, J=11.4, 7.9 Hz, 2H), 7.66-7.49 (m, 1H), 7.40 (d, J=7.3 Hz, 1H), 6.98 (d, J=8.6 Hz, 1H), 5.61 (d, J=6.8 Hz, 1H), 5.51-5.42 (m, 1H), 5.33-5.24 (m, 1H), 4.16-4.03 (m, 2H), 4.01-3.88 (m, 2H), 3.79 (d, J=8.9 Hz, 1H), 3.74-3.62 (m, 2H), 1.55 (d, J=4.0 Hz, 15H). ESI-MS m/z calc. 741.2338, found 741.22 (M+1)⁺; Retention time: 3.53 minutes, Method A.

### Example 88: 3-[4-[7-tert-butyl-5-(4-chloro-3-fluoro-phenyl)furo[3,2-b]pyridine-2-carbonyl]-3,3-dimethyl-2-oxo-piperazin-1-yl]-2,2-dimethyl-propanoic acid

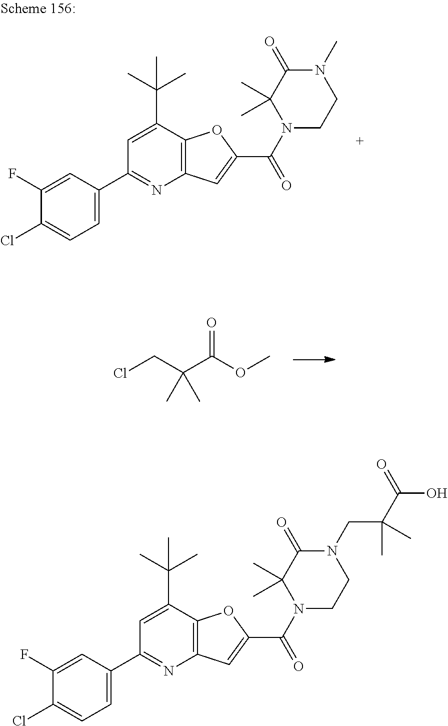

A solution of 4-[7-tert-butyl-5-(4-chloro-3-fluoro-phenyl)furo[3,2-b]pyridine-2-carbonyl]-3,3-dimethyl-piperazin-2-one (100 mg, 0.218 mmol) in DMF (1 mL) was treated with sodium hydride (9.5 mg, 0.40 mmol) and the solution was allowed to stir for 30 min. The mixture was then treated with a solution of methyl 3-chloro-2,2-dimethyl-propanoate (46.1 mg, 0.31 mmol) in DMF (0.3 mL). The mixture was then treated with tetrabutylammoniumiodide (5 mg, 0.014 mmol) and allowed to stir at RT overnight. It was treated with a further portion of NaH (60 mg) and allowed to stir for 30 min, followed by the addition of the alkyl chloride (120 mg). The mixture was heated at 60° C. overnight. The mixture was quenched with sat NH₄Cl and extracted with EtOAc (×3). The combined organic layers were washed (H₂O, brine), dried (Na₂SO₄) and concentrated in vacuo. The residue was dissolved in DMSO and purified by mass directed preparative HPLC. The appropriate tubes were lyophylized to give the desired product. ESI-MS m/z calc. 557.2093, found 558.2 (M+1)+; Retention time: 3.9 minutes using Method A; ¹H NMR (400 MHz, DMSO-d6) δ 12.35 (s, 1H), 8.11 (dd, J=11.0, 2.1 Hz, 1H), 8.01-7.94, (m. 1H), 7.78 (s, 1H), 7.65 (t, J=8.2 Hz, 1H), 7.53 (s, 1H), 3.75 (t, J=4.9 Hz, 2H), 3.57 (s, 2H), 3.50 (t, J=4.8 Hz, 2H), 1.65 (s, 6H), 1.46 (s, 9H), 1.06 (s, 6H).

### Example 89: 2-[4-[7-tert-butyl-5-(4-chloro-3-fluoro-phenyl)furo[3,2-b]pyridine-2-carbonyl]-3,3-dimethyl-piperazin-1-yl]-4-methyl-pyridine-3-carboxylic acid

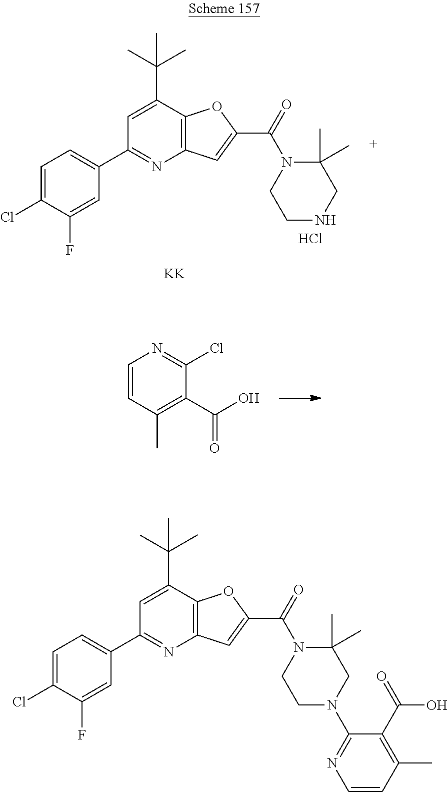 [SMILES: CC(C)(C)c1cc(-c2ccc(Cl)c(F)c2)nc2cc(C(=O)N3CCNCC3(C)C)oc12 ; Cl ; "KK" ; Cc1ccnc(Cl)c1C(=O)O ; Cc1ccnc(N2CCN(C(=O)c3cc4nc(-c5ccc(Cl)c(F)c5)cc(C(C)(C)C)c4o3)C(C)(C)C2)c1C(=O)O]

According to General Procedure 22A, a mixture of Intermediate KK (60 mg, 0.12 mmol), and 2-chloro-4-methyl-pyridine-3-carboxylic acid (21.4 mg, 0.125 mmol) in DMSO (1 mL) containing DIPEA (48.4 mg, 65 μL, 0.37 mmol) was heated at 120° C. overnight. The reaction mixture was treated with 0.2 mL formic acid and 0.1 mL H₂O. The solution was filtered and purified by mass directed prep HPLC. The appropriate tubes were lyophyllized to give the desired product. ESI-MS m/z calc. 578.2096, found 579.48 (M+1)+; Retention time: 3.82 minutes using Method A; ¹H NMR (400 MHz, DMSO-d6) δ 8.17 (dd, J=11.0, 2.1 Hz, 1H), 8.09-8.00 (m, 2H), 7.83 (s, 1H), 7.72 (t, J=8.1 Hz, 1H), 7.54 (s, 1H), 6.66 (d, J=5.1 Hz, 1H), 3.99 (t, J=5.4 Hz, 2H), 3.63 (s, 2H), 3.59-3.51 (m, 2H), 2.25 (s, 3H), 1.54 (s, 9H), 1.53 (s, 6H).

### Example 90: 4-[2-[4-[7-tert-butyl-5-(4-chloro-3-fluoro-phenyl)furo[3,2-b]pyridine-2-carbonyl]-3,3-dimethyl-piperazin-1-yl]-2-oxo-ethyl]tetrahydropyran-4-carboxylic acid

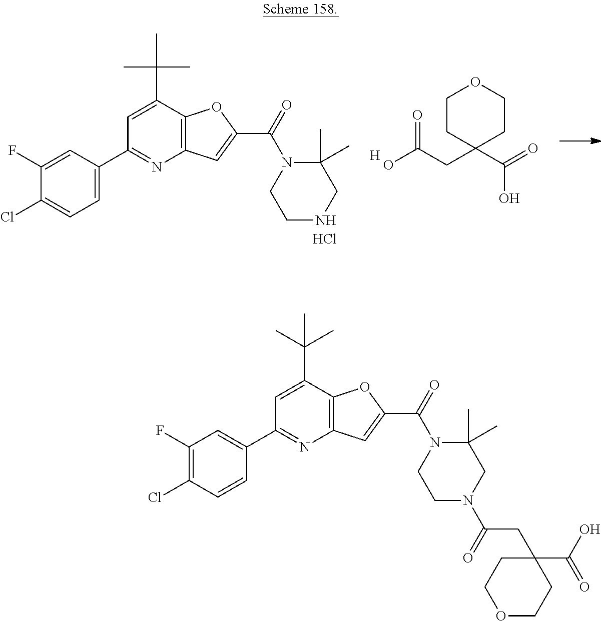

According to General Procedure 3, a solution of [7-tert-butyl-5-(4-chloro-3-fluoro-phenyl)furo[3,2-b]pyridin-2-yl]-(2,2-dimethylpiperazin-1-yl)methanone (Hydrochloride salt) (150 mg, 0.312 mmol) in DMF (2 mL) were added 4-(carboxymethyl)tetrahydropyran-4-carboxylic acid (65 mg, 0.35 mmol), DIPEA (280 μL, 1.61 mmol) and HATU (130.6 mg, 0.343 mmol). The mixture was stirred at rt for 2 h. It was then diluted with water, acidified with formic acid to pH 4, filtered. The solid was washed with water, dried in vacuo and purified using mass directed prep-HPLC to obtain 4-[2-[4-[7-tert-butyl-5-(4-chloro-3-fluoro-phenyl)furo[3,2-b]pyridine-2-carbonyl]-3,3-dimethyl-piperazin-1-yl]-2-oxo-ethyl]tetrahydropyran-4-carboxylic acid (125 mg, 64%). ESI-MS m/z calc. 613.2355, found 614.25 (M+1)+; Retention time: 3.67 minutes using Method A. ¹H NMR (400 MHz. Methanol-d4) δ 7.92 (dt, J=10.7, 2.3 Hz, 1H), 7.86-7.78 (m, 1H), 7.74 (d J=4.2 Hz, 1H), 7.57 (ddd, J=8.5, 7.6, 2.3 Hz, 1H), 7.41 (d, J=5.5 Hz, 1H), 4.04 (dt, J=19.1, 5.8 Hz, 2H), 3.89-3.55 (m 8H), 2.83 (s, 1H), 2.76 (s, 1H), 2.23-2.06 (m, 2H), 1.73-1.53 (m, 17H).

### Example 91: N-(benzenesulfonyl)-4-[7-tert-butyl-5-(4-chloro-3-fluoro-phenyl)furo[3,2-b]pyridine-2-carbonyl]-3,3-dimethyl-piperazine-1-carboxamide

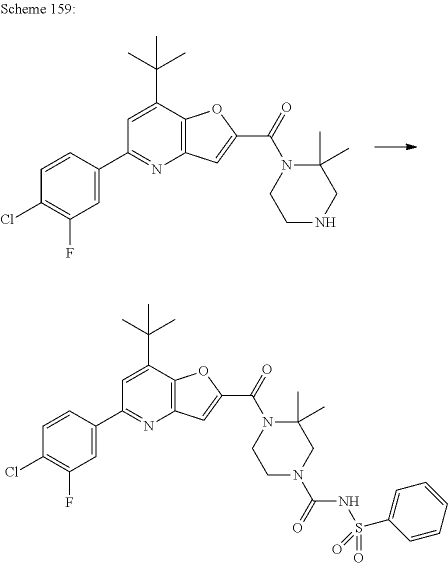 [SMILES: CC(C)(C)c1cc(-c2ccc(Cl)c(F)c2)nc2cc(C(=O)N3CCNCC3(C)C)oc12 ; CC(C)(C)c1cc(-c2ccc(Cl)c(F)c2)nc2cc(C(=O)N3CCN(C(=O)NS(=O)(=O)c4ccccc4)CC3(C)C)oc12]

7-tert-butyl-5-(4-chloro-3-fluoro-phenyl)furo[3,2-b]pyridin-2-yl I-(2,2-dimethylpiperazin-1-yl)methanone (Hydrochloride salt) (150 mg, 0.312 mmol) and N-(oxomethylene)benzenesulfonamide (68.6 mg, 50 μL, 0.375 mmol) were dissolved in DCM (1.6 mL) and NEt₃ (174.1 μL, 1.25 mmol) was added. The reaction mixture was stirred at RT for 16 h (ON) and then evaporated. The crude residue was dissolved in NMP, filtered and purified by UV directed reverse phase preparative HPCL. Desired fractions were combined and lyophilized to give N-(benzenesulfonyl)-4-[7-tert-butyl-5-(4-chloro-3-fluoro-phenyl)furo[3,2-b]pyridine-2-carbonyl]-3,3-dimethyl-piperazine-1-carboxamide (47 mg, 23%). ¹H NMR (400 MHz, DMSO-d6) δ 10.94 (s, 1H, br), 8.21-8.13 (m, 1H), 8.07-8.00 (m, 1H), 7.95-7.88 (m, 2H), 7.83 (s, 1H), 7.76-7.55 (m, 4H), 7.52 (s, 1H), 3.93-3.88 (m, 2H), 3.70-3.45 (m 4H), 1.53 (s, 9H), 1.46 (s, 6H). ESI-MS m/z calc. 626.1766, found 627.25 (M+1)+; Retention time: 4.1 minutes using Method A.

### Example 92: 4-[7-tert-butyl-5-(4-chloro-3-fluoro-phenyl)furo[3,2-b]pyridine-2-carbonyl]-3,3-dimethyl-piperazine-1-carboxamide

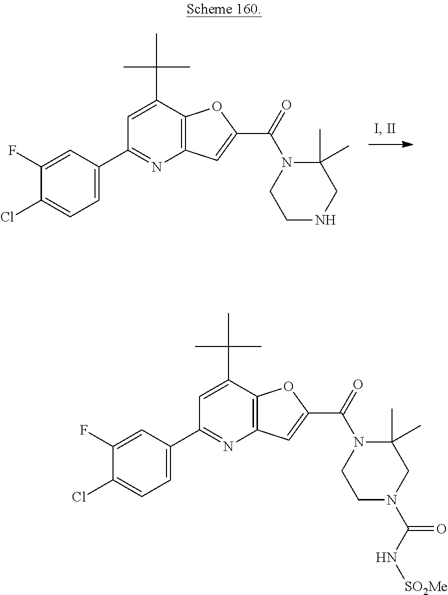 [SMILES: CC(C)(C)c1cc(-c2ccc(Cl)c(F)c2)nc2cc(C(=O)N3CCNCC3(C)C)oc12 ; CC(C)(C)c1cc(-c2ccc(Cl)c(F)c2)nc2cc(C(=O)N3CCN(C(=O)NS(C)(=O)=O)CC3(C)C)oc12]

### Step I: 4-(7-(tert-butyl)-5-(4-chloro-3-fluorophenyl)furo[3,2-b]pyridine-2-carbonyl)-3,3-dimethylpiperazine-1-carboxamide

To a stirred solution of [7-tert-butyl-5-(4-chloro-3-fluoro-phenyl)furo[3,2-b]pyridin-2-yl]-(2,2-dimethylpiperazin-1-yl)methanone (Hydrochloride salt) (500 mg, 1.04 mmol) and DIPEA (148.0 mg, 199.5 μL, 1.15 mmol) in anhydrous DCM (3.5 mL), was added trimethylsilylisocyanate (2.16 g, 2.54 mL, 18.74 mmol). The resulting reaction mixture was stirred at room temperature overnight. The solvent was then evaporated under vacuum to give the crude 4-[7-tert-butyl-5-(4-chloro-3-fluoro-phenyl)furo[3,2-b]pyridine-2-carbonyl]-3,3-dimethyl-piperazine-1-carboxamide (873 mg, 172%). ESI-MS m/z calc. 486.1834, found 487.2 (M+1)+; Retention time: 3.45 minutes using Method A.

### Step II: 4-[7-tert-butyl-5-(4-chloro-3-fluoro-phenyl)furo[3,2-b]pyridine-2-carbonyl]-3,3-dimethyl-N-methylsulfonyl-piperazine-1-carboxamide

[7-tert-butyl-5-(4-chloro-3-fluoro-phenyl)furo[3,2-b]pyridin-2-yl]-(2,2-dimethylpiperazin-1-yl)methanone (100 mg, 0.225 mmol) and TEA (27.36 mg, 37.7 μL, 0.270 mmol) were suspended in acetonitrile (1.0 mL) and CDI (36.5 mg, 0.225 mmol) was added at room temperature, followed by stirring until it becomes a white thick solid (2 hours). To the reaction mixture were added methanesulphonamide (64.3 mg, 0.676 mmol) and DBU (102.9 mg, 101.1 μL, 0.676 mmol). The reaction was then stirred to 60° C. overnight and then concentrated under reduced pressure. Saturated NH₄Cl and DCM were added to the residue and the phases were separated. The aqueous phase was washed with DCM and the combined organic layers were concentrated under reduced pressure. The crude residue was dissolved in NMP and purified by UV directed reverse phase HPLC to give 4-[7-tert-butyl-5-(4-chloro-3-fluoro-phenyl)furo[3,2-b]pyridine-2-carbonyl]-3,3-dimethyl-N-methylsulfonyl-piperazine-1-carboxamide (45 mg, 35%). ¹H NMR (400 MHz, DMSO-d6) δ 10.48 (s, 1H), 8.17 (dd, J=11.0, 2.1 Hz, 1H), 8.07-8.00 (m, 1H), 7.83 (s, 1H), 7.76-7.67 (m, 1H), 7.53 (s, 1H), 3.98-3.90 (m, 2H), 3.64-3.53 (m, 4H), 3.26 (s, 3H), 1.53 (s, 9H), 1.51 (s, 6H). ESI-MS m/z calc. 564.16095, found 567.1 (M+1)+; Retention time: 3.68 minutes using Method A.

### Example 93: 4-[4-[7-tert-butyl-5-(4-chloro-3-fluoro-phenyl)furo[3,2-b]pyridine-2-carbonyl]-3,3-dimethyl-piperazin-1-yl]pyridine-3-carboxylic acid

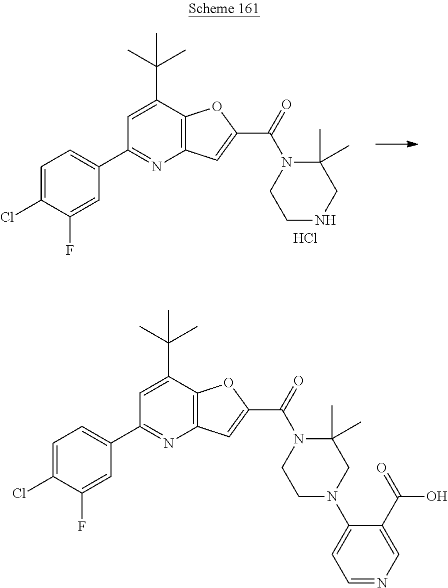 [SMILES: CC(C)(C)c1cc(-c2ccc(Cl)c(F)c2)nc2cc(C(=O)N3CCNCC3(C)C)oc12 ; Cl ; CC(C)(C)c1cc(-c2ccc(Cl)c(F)c2)nc2cc(C(=O)N3CCN(c4ccncc4C(=O)O)CC3(C)C)oc12]

A solution of [7-tert-butyl-5-(4-chloro-3-fluoro-phenyl)furo[3,2-b]pyridin-2-yl]-(2,2-dimethylpiperazin-1-yl)methanone (Hydrochloride salt) (125 mg, 0.260 mmol), in DMSO (2 mL) containing DIPEA (136.0 μL, 0.781 mmol) was heated at 120° C. overnight. The reaction mixture was added dropwise to 0.5M HCl and the mixture was extracted with EtOAc (×3). The combined organic layers were washed (H₂O, brine), dried (Na₂SO₄) and concentrated in vacuo. The aq mphase was basified to pH 5 and saturated with brine. It was extracted twice more with EtOAc and these were combined with the original organic phases. The solvent was again removed in vacuo and the residue was purified by mass directed HPLC to give 4-[4-[7-tert-butyl-5-(4-chloro-3-fluoro-phenyl)furo[3,2-b]pyridine-2-carbonyl]-3,3-dimethyl-piperazin-1-yl]pyridine-3-carboxylic acid (11.6 mg, 8%). ¹H NMR (400 MHz, DMSO-d6) δ 12.94 (s, 1H), 8.41 (s, 1H), 8.18 (d, J=6.0 Hz, 1H), 8.16-8.03 (m, 1H), 7.97 (dd, J=8.5, 2.1 Hz, 1H), 7.77 (s, 1H), 7.65 (t, J=8.1 Hz, 1H), 7.50 (s, 1H), 6.81 (d, J=6.1 Hz, 1H), 4.00 (d, J=5.5 Hz, 2H), 3.45 (s, 4H), 1.47 (s, 6H), 1.47 (s, 9H). ESI-MS m/z calc. 564.194, found 565.66 (M+1)+; Retention time: 3.19 minutes using Method A.

### Example 94: cis-(rac)-2-[4-[7-tert-butyl-5-(4-chloro-3-fluoro-phenyl)furo[3,2-b]pyridine-2-carbonyl]-3,3-dimethyl-piperazine-1-carbonyl]cyclohexanecarboxylic acid

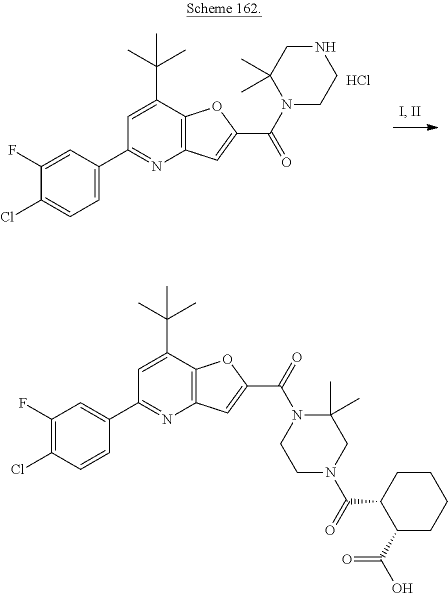 [SMILES: CC(C)(C)c1cc(-c2ccc(Cl)c(F)c2)nc2cc(C(=O)N3CCNCC3(C)C)oc12 ; Cl ; CC(C)(C)c1cc(-c2ccc(Cl)c(F)c2)nc2cc(C(=O)N3CCN(C(=O)[C@@H]4CCCC[C@@H]4C(=O)O)CC3(C)C)oc12]

### Step I

To a solution of [7-tert-butyl-5-(4-chloro-3-fluoro-phenyl)furo[3,2-b]pyridin-2-yl]-(2,2-dimethylpiperazin-1-yl)methanone (Hydrochloride salt) (150 mg, 0.312 mmol) in DMF (2 mL) were added (cis)-2-methoxycarbonylcyclohexanecarboxylic acid (69 mg, 0.37 mmol). DIPEA (270 μL, 1.55 mmol) and HATU (142.4 mg, 0.375 mmol). The mixture was stirred at RT for 2 h. The volatiles were removed under reduced pressure and the residue was purified by flash chromatography on silica gel eluting with EtOAc/hexanes 0-60% to obtain methyl (cis)-2-[4-[7-tert-butyl-5-(4-chloro-3-fluoro-phenyl)furo[3,2-b]pyridine-2-carbonyl]-3,3-dimethyl-piperazine-1-carbonyl]cyclohexanecarboxylate (130 mg, 68%) as a white solid. ESI-MS m/z calc. 611.2562, found: 612.05. Retention time: 2.23 minutes using Method C.

### Step II

To a solution of methyl (cis)-2-[4-[7-tert-butyl-5-(4-chloro-3-fluoro-phenyl)furo[3,2-b]pyridine-2-carbonyl]-3,3-dimethyl-piperazine-1-carbonyl]cyclohexanecarboxylate (130 mg, 0.212 mmol) in dioxane (3 mL) was added 1M KOH (1 mL, 1.0 mmol). The mixture was heated at 95° C. for 16 h. After cooling to RT, the mixture was neutralized with Resin Amberlite IR120 (H), filtered. The filtrate was concentrated to dryness and the residue was purified using mass directed reverse-phase Prep-HPLC to provide (cis)-2-[4-[7-tert-butyl-5-(4-chloro-3-fluoro-phenyl)furo[3,2-b]pyridine-2-carbonyl]-3,3-dimethyl-piperazine-1-carbonyl]cyclohexanecarboxylic acid (68 mg, 52%). ESI-LC-MS: calc. 598.2484, found: 599.45; RT=4.20 min using method A. ¹H NMR (400 MHz. Methanol-d4) δ 7.93 (dd, J=10.7, 2.0 Hz, 1H), 7.83 (dd, J=8.5, 2.0 Hz, 1H), 7.75 (d, J=2.1 Hz, 1H), 7.58 (t, J=8.0 Hz, 1H), 7.41 (d, J=3.2 Hz, 1H), 4.07-3.90 (m, 2H), 3.89-3.65 (m, 2H), 3.54-3.40 (m, 1H), 2.59-2.48 (m, 1H), 2.29 (t, J=12.7 Hz, 1H), 2.00-1.75 (m, 3H), 1.74-1.23 (m, 20H).

### Example 95: phosphonooxymethyl 4-[4-[7-tert-butyl-5-(4,4-dimethylcyclohexyl)furo[3,2-b]pyridine-2-carbonyl]-3,3-dimethyl-piperazine-1-carbonyl]-1-methyl-cyclohexanecarboxylate

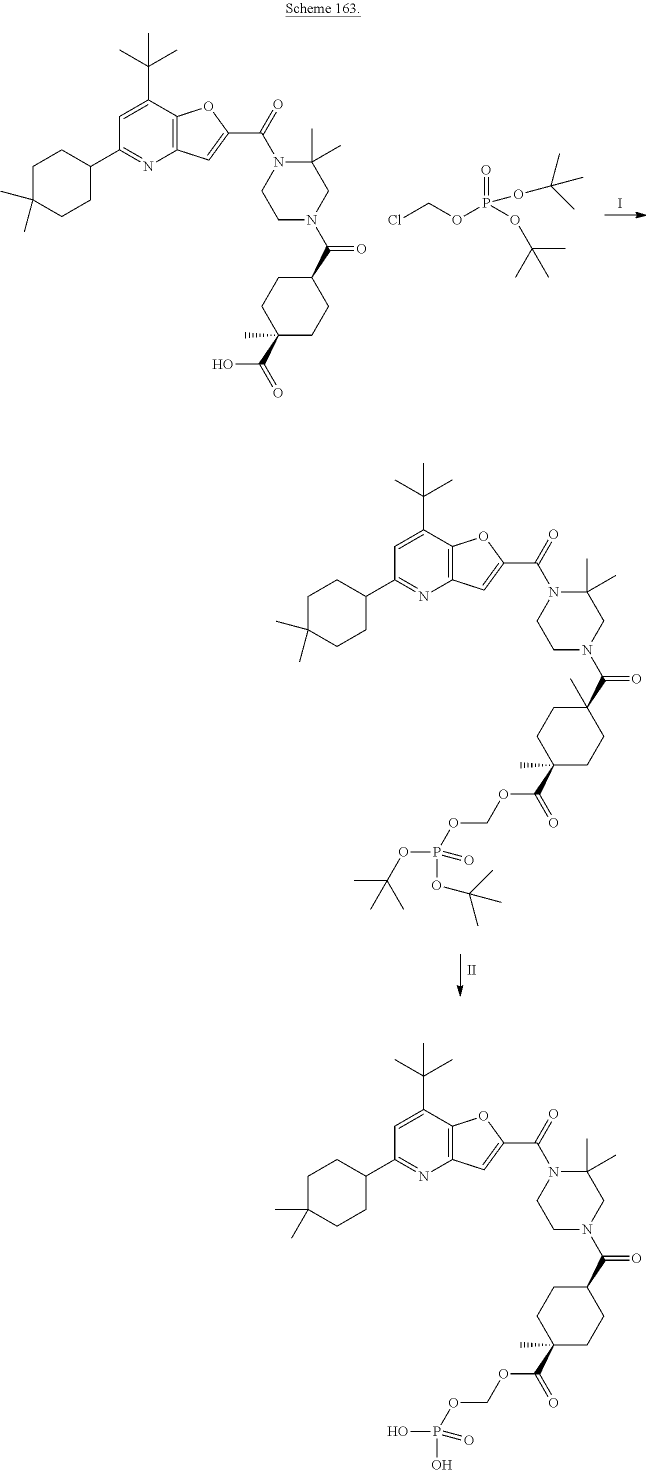

### Step I: di-tert-butoxyphosphoryloxymethyl 4-[4-[7-tert-butyl-5-(4,4-dimethylcyclohexyl)furo[3,2-b]pyridine-2-carbonyl]-3,3-dimethyl-piperazine-1-carbonyl]-1-methyl-cyclohexanecarboxylate

A mixture of 4-[4-[7-tert-butyl-5-(4,4-dimethylcyclohexyl)furo[3,2-b]pyridine-2-carbonyl]-3,3-dimethyl-piperazine-1-carbonyl]-1-methyl-cyclohexanecarboxylic acid (404.1 mg, 0.675 mmol), potassium iodide (152.1 mg, 0.916 mmol) and cesium carbonate (682.2 mg, 2.09 mmol) in DMSO (8 mL) was heated to 40° C. then di-tert-butyl chloromethyl phosphate (200 μL, 0.862 mmol) was added dropwise over 5 minutes. The reaction mixture was heated at that temperature for 3 h then cooled to 5° C. prior to being diluted with isopropyl acetate (8 mL). While keeping the temperature below 10° C., water (8 mL) was added dropwise. Layers were separated and the aqueous layer was extracted with iPrOAc. The combined organic layers were washed twice with water, dried over MgSO₄ and concentrated to dryness under reduced pressure to afford di-tert-butoxyphosphoryloxymethyl 4-[4-[7-tert-butyl-5-(4,4-dimethylcyclohexyl)furo[3,2-b]pyridine-2-carbonyl]-3,3-dimethyl-piperazine-1-carbonyl]-1-methyl-cyclohexanecarboxylate (550.8 mg, quantitative), which was used directly in the next step without further purification. ESI-MS m/z calc. 815.485, found 816.02 (M+1)+; Retention time: 1.26 minutes using method J (Ammonium bicarbonate).

### Step II: phosphonooxymethyl 4-[4-[7-tert-butyl-5-(4,4-dimethylcyclohexyl)furo[3,2-b]pyridine-2-carbonyl]-3,3-dimethyl-piperazine-1-carbonyl]-1-methyl-cyclohexanecarboxylate

A solution of ditert-butoxyphosphoryloxymethyl 4-[4-[7-tert-butyl-5-(4,4-dimethylcyclohexyl)furo[3,2-b]pyridine-2-carbonyl]-3,3-dimethyl-piperazine-1-carbonyl]-1-methyl-cyclohexanecarboxylate (653.2 mg, 0.8005 mmol) in IPA (6 mL) was heated to 55° C., then water (6 mL) was added dropwise until the solution was just about to turn hazy. The reaction mixture was heated at that temperature for 3 h, the allowed to cool to rt. The solvent (IPA) was removed under reduced pressure then the aqueous layer was extracted three times with MTBE. The combined organic layers were dried over MgSO4 and concentrated under reduced pressure. The residue was purified by mass-directed reverse phase HPLC to afford, upon lyophilization, the title compound phosphonooxymethyl 4-[4-[7-tert-butyl-5-(4,4-dimethylcyclohexyl)furo[3,2-b]pyridine-2-carbonyl]-3,3-dimethyl-piperazine-1-carbonyl]-1-methyl-cyclohexanecarboxylate (75.2 mg, 13%). 1H NMR (400 MHz, DMSO-d6) δ 7.42, 7.38 (s, 1H), 7.15, 7.14 (s, 1H), 5.45 (d, J=12.2 Hz, 2H), 3.96-3.81 (m, 2H), 3.80-3.72 (m, 1H), 3.68 (s, 1H), 3.61 (s, 1H), 3.51-3.37 (m, 1H), 2.77-2.55 (m, 1H), 2.21-2.07 (m, 2H), 1.89-1.16 (m, 30H), 1.12, 1.13 (s, 3H), 0.97 (s, 3H), 0.95 (s, 3H). ESI-MS m/z calc. 703.35974, found 704.73 (M+1)+; Retention time: 3.47 minutes using method A.

### Example 96: (4-amino-2,2-dimethyl-1-piperidyl)-[7-tert-butyl-5-(4-chloro-3-fluoro-phenyl)furo[3,2-b]pyridin-2-yl]methanone

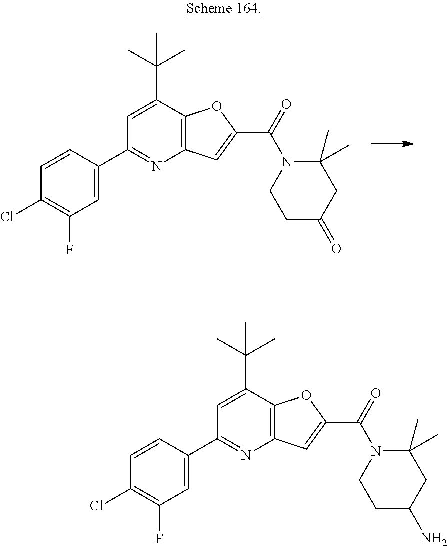 [SMILES: CC(C)(C)c1cc(-c2ccc(Cl)c(F)c2)nc2cc(C(=O)N3CCC(=O)CC3(C)C)oc12 ; CC(C)(C)c1cc(-c2ccc(Cl)c(F)c2)nc2cc(C(=O)N3CCC(N)CC3(C)C)oc12]

A mixture of 1-[7-tert-butyl-5-(4-chloro-3-fluoro-phenyl)furo[3,2-b]pyridine-2-carbonyl]-2,2-dimethyl-piperidin-4-one (350 mg, 0.766 mmol), Titanium(IV) isopropoxide (435.4 mg, 452.1 μL, 1.53 mmol) and Ammonia in MeOH (547.1 μL of 7 M, 3.830 mmol) were stirred under argon in a capped flask at rt for 6.5 h. NaBH₄ (43.5 mg, 46.00 μL, 1.15 mmol) was added and the reaction was stirred at r.t. for 2 days. The reaction was quenched by pouring into ammonium hydroxide 10% aqueous solution. DCM was added and the organic phase was separated. The aqueous phase was washed with DCM and combined filtrates were concentrated to give upon trituration crude (4-amino-2,2-dimethyl-1-piperidyl)-[7-tert-butyl-5-(4-chloro-3-fluoro-phenyl)furo[3,2-b]pyridin-2-yl]methanone (Formate (0.5)) (307 mg, 81%). ESI-MS m/z calc. 457.19324, found 456.15 (M+1)+; Retention time: 2.72 minutes using Method A. The material was further purified by UV directed reverse phase HPLC to give (4-amino-2,2-dimethyl-1-piperidyl)-[7-tert-butyl-5-(4-chloro-3-fluoro-phenyl)furo[3,2-b]pyridin-2-yl]methanone (95 mg). ¹H NMR (400 MHz, DMSO-d6) δ 8.17 (dd, J=11.0, 2.1 Hz, 1H), 8.07-8.00 (m, 1H), 7.84 (s, 1H), 7.77-7.68 (m, 1H), 7.53 (s, 1H), 7.26 (s, 2H, br), 3.90-3.79 (m, 1H), 3.47-3.17 (m, 2H), 2.12-1.98 (m, 1H), 1.87-1.77 (m, 1H), 1.71-1.49 (m, 14H), 1.45 (s, 3H).

ESI-MS m/z calc. 457.19324, found 456.15 (M+1)+; Retention time: 2.72 minutes using method A.

### Example 97: 1-[l-[7-tert-butyl-5-(4-chloro-3-fluoro-phenyl)furo[3,2-b]pyridine-2-carbonyl]-2,2-dimethyl-4-piperidyl]-3-methylsulfonyl-urea

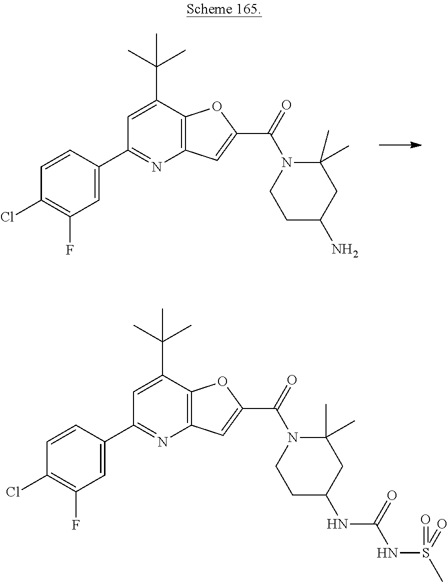 [SMILES: CC(C)(C)c1cc(-c2ccc(Cl)c(F)c2)nc2cc(C(=O)N3CCC(N)CC3(C)C)oc12 ; CC(C)(C)c1cc(-c2ccc(Cl)c(F)c2)nc2cc(C(=O)N3CCC(NC(=O)NS(C)(=O)=O)CC3(C)C)oc12]

A solution of (4-amino-2,2-dimethyl-1-piperidyl)-[7-tert-butyl-5-(4-chloro-3-fluoro-phenyl)furo[3,2-b]pyridin-2-yl]methanone (100 mg, 0.218 mmol) and TEA (46.41 mg, 63 μL, 0.459 mmol) in Acetonitrile (1. mL) was treated with CDI (35.41 mg, 0.218 mmol) at room temperature. The reaction was stirred for 4 hours and then, Methanesulphonamide (62.32 mg, 0.655 mmol), followed by DBU (99.8 mg, 98 μL, 0.655 mmol) were added. The reaction mixture was then heated to 60° C. and stirred for overnight at that temperature. The reaction was diluted in NMP and purified by UV directed reverse phase HPLC to give 1-[1-[7-tert-butyl-5-(4-chloro-3-fluoro-phenyl)furo[3,2-b]pyridine-2-carbonyl]-2,2-dimethyl-4-piperidyl]-3-methylsulfonyl-urea (44.8 mg, 35%). ¹H NMR (400 MHz, DMSO-d6) δ 10.02 (s, 1H), 8.17 (dd, J=11.0, 2.0 Hz, 1H), 8.03 (dd, J=8.5, 2.0 Hz, 1H), 7.83 (s, 1H), 7.76-7.67 (m, 1H), 7.52 (s, 1H), 6.58 (d, J=7.4 Hz, 1H), 3.84 (s, 2H), 3.32-3.27 (m, 1H), 3.23 (s, 3H), 2.14-1.93 (m, 1H), 1.83-1.74 (m, 1H), 1.71-1.50 (m, 14H), 1.47 (s, 3H). ESI-MS m/z calc. 578.1766, found 581.2 (M+1)+; Retention time: 3.81 minutes using Method A.

### Protocol for Testing PAR2 Compounds in ANTAGONIST Mode Using the Ca FLIPR^{TETRA} Assay (384 Well).

23000 HT-29 cells (ATCC# HTB-38) were plated in 384-well black, transparent-flat bottom plates and incubated in 25 uL of 1% FBS/McCoy's media ON at 37° C. under an atmosphere of 5% CO₂. The following day, 25 uL FLIPR calcium 5 Molecular Devices assay reagent (Cat: R-8186) was added. Plates were incubated at 37° C. and r. t. for 45 min and 15 min, respectively. Compounds dilutions were prepared in 100% DMSO using a Biomek FX and diluted 20-fold in HBSS and then 5.5 uL was added directly to the cells by the FLIPR^{TETRA} (10× dilution) using FLIPR Tetra Pipette tips, black, non-sterile, 384 Molecular Devices (Cat: 9000-0764).

A first read was performed to determine whether the test compounds on their own can activate Ca²⁺ responses. The cells were incubated with the compounds for 30 min at r. t. and then read in ANTAGONIST mode in the same plate by stimulating with 6.2 uL of a fixed concentration of the activator: final concentrations 8 μM SLIGKV, 1 U/mL Thrombin, 3.1 U/mL trypsin, or 0.6 μM UTP.

TABLE 1
Compound Analytical Data

| Com- | LCMS | | | | |
|---|---|---|---|---|---|
| pound | Retention | | LCMS | | General |
| Number | Time | M + 1 | Method | NMR | Procedure |
| 1 | 1.71 | 482.46 | C | ¹H NMR (400 MHz, Chloroform-d) δ | N/A |
| | | | | 7.99-7.91 (m, 2H), 7.53 (s, 1H), 7.39 | |
| | | | | (s, 1H), 7.20-7.11 (m, 2H), 4.03-3.71 | |
| | | | | (m, 6H), 3.44 (hept, J = 6.7 Hz, 1H), | |
| | | | | 1.64 (s, 7H), 1.53 (s, 6H), 1.44 (d, J = | |
| | | | | 7.0 Hz, 6H). | |
| 2 | 2.16 | 488.39 | C | N/A | 1 |
| 3 | 3.97 | 454.58 | D | ¹H NMR (400 MHz, DMSO-d6) δ 8.19- | 3 |
| | | | | 8.07 (m, 2H), 7.89 (s, 1H), 7.46 (s, | |
| | | | | 1H), 7.36-7.23 (m, 2H), 5.40 (s, 1H), | |
| | | | | 4.26-3.39 (m, 6H), 2.56 (s, 3H), 1.55- | |
| | | | | 1.44 (m, 6H), 1.32 (s, 6H). | |
| 4 | 4.21 | 468.52 | D | ¹H NMR (400 MHz, DMSO-d6) δ 8.21- | 3 |
| | | | | 8.08 (m, 2H), 7.88 (s, 1H), 7.47 (s, | |
| | | | | 1H), 7.38-7.23 (m, 2H), 5.41 (s, 1H), | |
| | | | | 4.27-3.38 (m, 6H), 2.95 (q, J = 7.7 | |
| | | | | Hz, 2H), 1.59-1.41 (m, 6H), 1.39- | |
| | | | | 1.27 (m, 9H). | |
| 5 | 4.18 | 480.32 | D | ¹H NMR (400 MHz, DMSO-d6) δ 8.22- | 3 |
| | | | | 8.07 (m, 2H), 7.56 (s, 1H), 7.45 (s, | |
| | | | | 1H), 7.36-7.18 (m, 2H), 5.46-5.26 | |
| | | | | (m, 1H), 4.22-3.15 (m, 6H), 2.36- | |
| | | | | 2.30 (m, 1H), 1.58-1.39 (m, 6H), 1.32 | |
| | | | | (s, 6H), 1.26-1.13 (m, 4H). | |
| 6 | 2.02 | 496.79 | C | ¹H NMR (400 MHz, DMSO-d6) δ 8.23- | 1 |
| | | | | 8.14 (m, 2H), 7.88 (s, 1H), 7.50 (s, | |
| | | | | 1H), 7.33 (t, J = 8.9 Hz, 2H), 3.86 (s, | |
| | | | | 2H), 3.58-3.39 (m, 5H), 1.49 (s, 6H), | |
| | | | | 1.42 (d, J = 7.7 Hz, 15H). | |
| 7 | 1.47 | 410.69 | C | ¹H NMR (400 MHz, DMSO-d6) δ 8.25- | 1 |
| | | | | 8.14 (m, 3H), 7.88 (s, 1H), 7.57 (s, | |
| | | | | 1H), 7.37-7.29 (m, 2H), 3.81-3.68 | |
| | | | | (m, 2H), 3.48-3.41 (m, 3H), 1.69 (s, | |
| | | | | 6H), 1.41 (d, J = 6.9 Hz, 6H). | |
| 8 | 1.62 | 494.45 | C | ¹H NMR (400 MHz, DMSO-d6) δ 8.21- | 3 |
| | | | | 8.16 (m, 2H), 7.88 (s, 1H), 7.51-7.49 | |
| | | | | (m, 1H), 7.37-7.29 (m, 2H), 6.03- | |
| | | | | 5.95 (m, 1H), 3.96 (t, J = 5.8 Hz, 1H), | |
| | | | | 3.81 (s, 1H), 3.74 (s, 1H), 3.62 (s, 1H), | |
| | | | | 3.53-3.47 (m, 1H), 3.47-3.38 (m, | |
| | | | | 1H). 2.62-2.54 (m, 3H), 2.11-1.98 | |
| | | | | (m, 2H), 1.85-1.71 (m, 1H), 1.55- | |
| | | | | 1.46 (m, 7H), 1.42 (d, J = 6.9 Hz, 6H). | |
| 9 | 1.56 | 463.42 | C | ¹H NMR (400 MHz, DMSO-d6) δ 8.22- | 3 |
| | | | | 8.15 (m, 2H), 7.88 (s, 1H), 7.53-7.49 | |
| | | | | (m, 1H), 7.37-7.29 (m, 2H), 4.08 (d, J = | |
| | | | | 1.6 Hz, 2H), 3.96 (dt, J = 18.5, 5.8 Hz, | |
| | | | | 2H), 3.70-3.51 (m, 4H), 3.49-3.39 (m, | |
| | | | | 1H), 1.57-1.47 (m, 6H), 1.42 (d, J = 6.9 | |
| | | | | Hz, 6H). | |
| 10 | 1.60 | 494.45 | C | ¹H NMR (400 MHz, DMSO-d6) δ 8.18 | 3 |
| | | | | (dd, J = 8.8, 5.5 Hz, 2H), 7.88 (s, 1H), | |
| | | | | 7.54-7.46 (m, 1H), 7.33 (t, J = 8.7 Hz, | |
| | | | | 2H), 4.77-4.62 (m, 1H), 3.99-3.90 | |
| | | | | (m, 2H), 3.84-3.69 (m, 3H), 3.64 (s, | |
| | | | | 1H), 3.55-3.38 (m, 1H), 3.31-3.30 | |
| | | | | (m, 2H), 2.44-2.40 (m, 1H), 2.10- | |
| | | | | 1.99 (m, 2H), 1.94-1.79 (m, 1H), 1.53 | |
| | | | | (d, J = 2.6 Hz, 3H), 1.49 (s, 3H), 1.45- | |
| | | | | 1.39 (m, 6H). | |
| 11 | 1.77 | 500.8 | C | ¹H NMR (400 MHz, DMSO-d6) δ 8.15 | 3 |
| | | | | (s, 2H), 7.84 (d, J = 19.8 Hz, 1H), 7.48 | |
| | | | | (d, J = 20.9 Hz, 6H), 7.30 (t, J = 8.5 | |
| | | | | Hz, 2H), 4.00 (s, 1H), 3.88-3.62 (m, | |
| | | | | 3H), 3.52 (s, 1H), 3.41 (s, 1H), 1.57 (s, | |
| | | | | 2H), 1.45-1.20 (m, 10H). | |
| 12 | 1.70 | 496.79 | C | ¹H NMR (400 MHz, DMSO-d6) δ 8.23- | 3 |
| | | | | 8.15 (m, 2H), 7.88 (d, J = 1.9 Hz, 1H), | |
| | | | | 7.51 (d, J = 11.5 Hz, 1H), 7.37-7.30 | |
| | | | | (m, 2H), 4.80 (dd, J = 7.5, 6.2 Hz, 1H), | |
| | | | | 4.13-3.89 (m, 3H), 3.88-3.38 (m, 5H), | |
| | | | | 2.02-1.82 (m, 1H), 1.58-1.46 (m, 6H), | |
| | | | | 1.47-1.39 (m, 6H), 0.91-0.82 (m, 6H). | |
| 13 | 1.65 | 491.71 | C | ¹H NMR (400 MHz, DMSO-d6) δ 8.80 | 3 |
| | | | | (t, J = 2.0 Hz, 1H), 8.23-8.14 (m, 2H), | |
| | | | | 7.88 (d, J = 7.0 Hz, 1H), 7.54 (d, J = | |
| | | | | 3.8 Hz, 1H), 7.38-7.29 (m, 2H), 7.13- | |
| | | | | 7.00 (m, 1H), 4.09-4.02 (m, 1H), 4.02- | |
| | | | | 3.97 (m, 1H), 3.96-3.89 (m, 1H), | |
| | | | | 3.82 (s, 1H), 3.78 (s, 1H), 3.72 (t, J = | |
| | | | | 5.7 Hz, 1H), 3.49-3.36 (m, 1H), 1.58 | |
| | | | | (s, 3H), 1.50 (s, 3H), 1.47-1.36 (m, 6H). | |
| 14 | 1.84 | 480.78 | C | ¹H NMR (400 MHz, DMSO-d6) δ 8.21- | 3 |
| | | | | 8.15 (m, 2H), 7.88 (s, 1H), 7.51 (s, 1H), | |
| | | | | 7.37-7.30 (m, 2H), 3.95-3.88 (m, 2H), | |
| | | | | 3.77-3.73 (m, 2H), 3.69-3.59 (m, 2H), | |
| | | | | 3.48-3.39 (m, 1H), 1.52 (s, 6H), 1.42 | |
| | | | | (d, J = 6.9 Hz, 6H), 1.23 (s, 9H). | |
| 15 | 1.49 | 468.74 | C | ¹H NMR (400 MHz, DMSO-d6) δ 8.22- | 3 |
| | | | | 8.15 (m, 2H), 7.88 (s, 1H), 7.53-7.49 | |
| | | | | (m, 1H), 7.37-7.30 (m, 2H), 5.02 (dd, | |
| | | | | J = 10.5, 7.2 Hz, 1H), 4.56-4.34 (m, | |
| | | | | 1H), 4.01-3.84 (m, 3H), 3.73 (s, 1H), | |
| | | | | 3.65 (s, 1H), 3.57-3.47 (m, 1H), 3.47- | |
| | | | | 3.37 (m, 1H), 1.53 (s, 3H), 1.51-1.47 | |
| | | | | (m, 3H), 1.42 (d, J = 6.9 Hz, 6H), 1.25- | |
| | | | | 1.18 (m, 3H). | |
| 16 | 1.81 | 484.75 | C | ¹H NMR (400 MHz, DMSO-d6) δ 8.22- | 3 |
| | | | | 8.16 (m, 2H), 7.88 (d, J = 0.6 Hz, 1H), | |
| | | | | 7.51 (s, 1H), 7.37-7.29 (m, 2H), 4.04- | |
| | | | | 3.84 (m, 4H), 3.70 (s, 1H), 3.53 (s, 1H), | |
| | | | | 3.48-3.38 (m, 1H), 1.61 (s, 3H), 1.58- | |
| | | | | 1.48 (m, 8H), 1.42 (d, J = 7.0 Hz, 6H). | |
| 17 | 1.52 | 468.46 | C | ¹H NMR (400 MHz, DMSO-d6) δ 8.22- | 3 |
| | | | | 8.15 (m, 2H), 7.88 (s, 1H), 7.51 (d, J = | |
| | | | | 6.5 Hz, 1H), 7.37-7.29 (m, 2H), 4.15- | |
| | | | | 4.11 (m, 2H), 3.99-3.90 (m, 2H), 3.69- | |
| | | | | 3.63 (m, 2H), 3.58 (s, 1H), 3.55-3.49 | |
| | | | | (m, 1H), 3.47-3.39 (m, 1H), 3.33-3.29 | |
| | | | | (m, 3H), 1.51 (d, J = 9.9 Hz, 6H), 1.42 | |
| | | | | (d, J = 6.9 Hz, 6H). | |
| 18 | 1.87 | 524.83 | C | ¹H NMR (400 MHz, DMSO-d6) δ 8.22- | 3 |
| | | | | 8.15 (m, 2H), 7.88 (s, 1H), 7.51 (d, J = | |
| | | | | 6.5 Hz, 1H), 7.37-7.29 (m, 2H), 4.95 | |
| | | | | (d, J = 7.4 Hz, 1H), 4.75 (d, J = 7.7 Hz, | |
| | | | | 1H), 4.45-4.32 (m, 1H), 4.02-3.60 | |
| | | | | (m, 5H), 3.55-3,38 (m, 2H), 1.62- | |
| | | | | 1.47 (m, 6H), 1.45-1.35 (m, 7H), 1.00- | |
| | | | | 0.92 (m, 9H). | |
| 19 | 1.70 | 478.77 | C | ¹H NMR (400 MHz DMSO-d6) δ 8.22- | 3 |
| | | | | 8.15 (m, 2H), 7.88 (s, 1H), 7.51 (s, | |
| | | | | 1H), 7.37-7.29 (m, 2H), 3.97-3.87 | |
| | | | | (m, 2H), 3.87-3.48 (m, 3H), 3.48- | |
| | | | | 3.38 (m, 1H), 1.53 (s, 6H), 1.42 (d, J = | |
| | | | | 6.9 Hz, 6H), 1.27 (s, 3H), 0.88-0.80 | |
| | | | | (m, 2H), 0.61-0.55 (m, 2H). | |
| 20 | 1.68 | 462.67 | C | ¹H NMR (400 MHz, DMSO-d6) δ 8.22- | 3 |
| | | | | 8.15 (m, 2H), 7.90-7.86 (m, 1H), 7.54- | |
| | | | | 7.49 (m, 1H), 7.33 (td, J = 8.9, 1.4 | |
| | | | | Hz, 2H), 3.93 (t, J = 5.8 Hz, 2H), 3.83 | |
| | | | | (d, J = 5.1 Hz, 2H), 3.64 (s, 1H), 3.59- | |
| | | | | 3.50 (m, 1H), 3.48-3.38 (m, 1H), 2.08- | |
| | | | | 2.03 (m, 3H), 1.51 (d, J = 18.5 Hz, | |
| | | | | 6H), 1.42 (dd, J = 7.0, 4.5 Hz, 6H). | |
| 21 | 1.90 | 494.83 | C | ¹H NMR (400 MHz, DMSO-d6) δ 8.20- | 3 |
| | | | | 8.11 (m, 2H), 7.87-7.83 (m, 1H), 7.51- | |
| | | | | 7.44 (m, 1H), 7.35-7.26 (m, 2H), | |
| | | | | 3.88 (q, J = 7.4, 6.8 Hz, 2H), 3.74- | |
| | | | | 3.60 (m, 3H), 3.52-3.34 (m, 2H), 2.26 | |
| | | | | (s, 1H), 2.21-2.15 (m, 1H), 1.54-1.43 | |
| | | | | (m, 6H), 1.43-1.31 (m, 6H), 1.04- | |
| | | | | 0.94 (m, 9H). | |
| 22 | 1.83 | 492.82 | C | ¹H NMR (400 MHz, DMSO-d6) δ 8.22- | 3 |
| | | | | 8.15 (m, 2H), 7.90-7.87 (m, 1H), 7.54- | |
| | | | | 7.49 (m, 1H), 7.38-7.29 (m, 2H), | |
| | | | | 4.02-3.74 (m, 4H), 3.72-3.62 (m, | |
| | | | | 1H), 3.58-3.50 (m, 1H), 3.48-3.38 | |
| | | | | (m, 1H), 1.85-1.65 (m, 1H), 1.61- | |
| | | | | 1.37 (m, 12H), 1.19 (s, 3H), 1.00 (s, | |
| | | | | 4H), 0.68 (dd, J = 7.8, 3.9 Hz, 1H). | |
| 23 | 1.50 | 524.83 | C | ¹H NMR (400 MHz, DMSO-d6) δ 8.22- | 3 |
| | | | | 8.15 (m, 2H), 7.88 (s, 1H), 7.51 (s, | |
| | | | | 1H), 7.39-7.29 (m, 2H), 5.63-5.53 | |
| | | | | (m, 1H), 4.12 (s, 2H), 3.96 (s, 1H), | |
| | | | | 3.82 (s, 1H), 3.66 (d, J = 12.0 Hz, 5H), | |
| | | | | 3.55-3.37 (m, 2H), 1.97 (s, 2H), 1.61 | |
| | | | | (d, J = 13.4 Hz, 2H), 1.53 (s, 6H), 1.42 | |
| | | | | (d, J = 7.0 Hz, 6H). | |
| 24 | 1.51 | 438.78 | C | ¹H NMR (400 MHz, DMSO-d6) δ 8.22- | 3 |
| | | | | 8.14 (m, 2H), 7.90-7.87 (m, 1H), 7.54- | |
| | | | | 7.48 (m, 1H), 7.37-7.29 (m, 2H), | |
| | | | | 3.96 (t, J = 5.6 Hz, 1H), 3.93-3.86 (m, | |
| | | | | 1H), 3.68 (t, J = 5.6 Hz, 1H), 3.63 (d, | |
| | | | | J = 3.3 Hz, 2H), 3.53-3.47 (m, 1H), | |
| | | | | 3.47-3.37 (m, 1H), 2.08-1.99 (m, 3H), | |
| | | | | 1.56-1.47 (m, 6H), 1.46-1.38 (m, 6H). | |
| 25 | 1.49 | 422.36 | C | ¹H NMR (400 MHz, DMSO-d6) δ 8.21- | 1 |
| | | | | 8.09 (m, 3H), 7.74 (s, 1H), 7.53 (s, 1H), | |
| | | | | 7.34-7.23 (m, 2H), 3.78-3.65 (m, 2H), | |
| | | | | 1.66 (s, 6H), 1.53 (s, 3H), 1.28-1.21 (m, | |
| | | | | 2H), 0.93-0.83 (m, 2H). | |
| 26 | 1.55 | 492.68 | C | ¹H NMR (400 MHz, DMSO-d6) δ 8.17- | 3 |
| | | | | 8.11 (m, 2H), 7.73 (s, 1H), 7.47 (s, | |
| | | | | 1H), 7.33-7.24 (m, 2H), 6.34 (s, 1H), | |
| | | | | 4.26-3.34 (m, 5H), 1.54 (s, 3H), 1.49 | |
| | | | | (s, 6H), 1.30-1.23 (m, 2H), 0.97-0.74 | |
| | | | | (m, 6H). | |
| 27 | 1.59 | 494.73 | C | ¹H NMR (400 MHz, DMSO-d6) δ 8.16- | 3 |
| | | | | 8.08 (m, 2H), 7.71 (s, 1H), 7.44 (s, | |
| | | | | 1H), 7.28 (t, J = 8.9 Hz, 2H), 4.18- | |
| | | | | 3.67 (m, 6H), 1.52 (s, 3H), 1.47 (d, J = | |
| | | | | 12.3 Hz, 7H), 1.31 (s, 6H), 1.24 (s, | |
| | | | | 2H), 0.93-0.86 (m, 2H). | |
| 28 | 1.60 | 494.78 | C | ¹H NMR (400 MHz, DMSO-d6) δ 8.21- | 3 |
| | | | | 8.09 (m, 2H), 7.74 (d, J = 2.8 Hz, 1H), | |
| | | | | 7.47 (d, J = 9.6 Hz, 1H), 7.34-7.22 (m, | |
| | | | | 2H), 4.94-4.81 (m, 1H), 4.30-4.07 (m, | |
| | | | | 1H), 3.91 (q, J = 5.2 Hz, 2H), 3.78-3.39 | |
| | | | | (m, 4H), 1.70-1.56 (m, 1H), 1.54 (s, | |
| | | | | 3H), 1.52-1.41 (m, 7H), 1.33-1.21 (m, | |
| | | | | 2H), 0.94-0.90 (m, 2H), 0.86 (t, J = 7.3 | |
| | | | | Hz, 3H). | |
| 29 | 1.60 | 494.73 | C | ¹H NMR (400 MHz, DMSO-d6) δ 8.20- | 3 |
| | | | | 8.11 (m, 2H), 7.74 (d, J = 3.4 Hz, 1H), | |
| | | | | 7.47 (d, J = 10.3 Hz, 1H), 7.35-7.24 | |
| | | | | (m, 2H), 4.22 (dq, J = 35.9, 6.5 Hz, | |
| | | | | 1H), 3.92 (t, J = 5.9 Hz, 2H), 3.88- | |
| | | | | 3.40 (m, 4H), 3.18 (d, J = 3.0 Hz, 3H), | |
| | | | | 1.54 (s, 3H), 1.52-1.45 (m, 6H), 1.30- | |
| | | | | 1.24 (m, 2H), 1.21 (dd, J = 6.6, 3.1 Hz, | |
| | | | | 3H), 0.94-0.88 (m, 2H). | |
| 30 | 1.62 | 506.73 | C | ¹H NMR (400 MHz, DMSO-d6) δ 8.19- | 3 |
| | | | | 8.09 (m, 2H), 7.73 (d, J = 2.2 Hz, 1H), | |
| | | | | 7.47 (d, J = 8.3 Hz, 1H), 7.33-7.24 | |
| | | | | (m, 2H), 4.71-4.60 (m, 1H), 3.94-3.36 | |
| | | | | (m, 8H), 2.07-1.97 (m, 2H), 1.91-1.73 | |
| | | | | (m, 2H), 1.54 (s, 3H), 1.52-1.43 (m, | |
| | | | | 6H), 1.28-1.24 (m, 2H), 0.94-0.87 | |
| | | | | (m, 2H). | |
| 31 | 2.71 | 506.09 | H | N/A | 3 |
| 32 | 1.71 | 508.78 | C | ¹H NMR (400 MHz, DMSO-d6) δ 8.18- | 3 |
| | | | | 8.12 (m, 2H), 7.74 (d, J = 3.5 Hz, 1H), | |
| | | | | 7.47 (d, J = 11.4 Hz, 1H), 7.33-7.24 | |
| | | | | (m, 2H), 4.10-3.42 (m, 6H), 1.88 (m, | |
| | | | | 1H), 1.54 (s, 3H), 1.51-1.43 (m, 6H), | |
| | | | | 1.29-1.24 (m, 2H), 0.94-0.88 (m, | |
| | | | | 2H), 0.88-0.78 (m, 6H). | |
| 33 | 1.55 | 520.4 | C | ¹H NMR (400 MHz, DMSO-d6) δ 8.21- | 3 |
| | | | | 8.08 (m, 2H), 7.78-7.68 (m, 1H), 7.50- | |
| | | | | 7.45 (m, 1H), 7.35-7.22 (m, 2H), 5.56- | |
| | | | | 5.34 (m, 1H), 4.06-3.39 (m, 6H), 2.46- | |
| | | | | 2.09 (m, 1H), 1.54 (s, 3H), 1.53-1.45 | |
| | | | | (m, 6H), 1.33-1.22 (m, 2H), 0.95-0.86 | |
| | | | | (m, 2H). | |
| 34 | 1.79 | 523.43 | C | ¹H NMR (400 MHz, DMSO-d6) δ 8.20- | 3 |
| | | | | 8.09 (m, 2H), 7.73 (d, J = 2.8 Hz, 1H), | |
| | | | | 7.47 (d, J = 8.9 Hz, 1H), 7.34-7.23 | |
| | | | | (m, 2H), 4.88 (dd, J = 21.4, 7.3 Hz, | |
| | | | | 1H), 4.35-4.14 (m, 1H), 4.00-3.41 | |
| | | | | (m, 6H), 1.54 (s, 3H), 1.51-1.43 (m, | |
| | | | | 6H), 1.37-1.14 (m, 6H), 0.95-0.87 | |
| | | | | (m, 2H), 0.84 (q, J = 7.1 Hz, 3H). | |
| 35 | 1.69 | 508.78 | C | ¹H NMR (400 MHz, DMSO-d6) δ 8.19- | 3 |
| | | | | 8.10 (m, 2H), 7.73 (s, 1H), 7.46 (s, | |
| | | | | 1H), 7.34-7.23 (m, 2H), 5.32-5.13 | |
| | | | | (m, 1H), 4.25-3.38 (m, 5H), 1.57- | |
| | | | | 1.39 (m, 9H), 1.31-1.22 (m, 5H), 0.92- | |
| | | | | 0.88 (m, 2H), 0.79 (t, J = 7.5 Hz, 3H). | |
| 36 | 1.79 | 522.78 | C | ¹H NMR (400 MHz, DMSO-d6) δ 8.19- | 3 |
| | | | | 8.10 (m, 2H), 7.73 (d, J = 1.8 Hz, 1H), | |
| | | | | 7.47 (d, J = 6.5 Hz, 1H), 7.34-7.23 | |
| | | | | (m, 2H), 4.90 (dd, J = 67.1, 7.4 Hz, | |
| | | | | 1H), 4.36-4.20 (m, 1H), 4.04-3.43 | |
| | | | | (m, 6H), 1.81-1.64 (m, 1H), 1.54 (s, | |
| | | | | 3H), 1.52-1.44 (m, 6H), 1.43-1.31 | |
| | | | | (m, 2H), 1.29-1.23 (m, 2H), 0.92- | |
| | | | | 0.83 (m, 8H). | |
| 37 | 1.56 | 524.74 | C | ¹H NMR (400 MHz, DMSO-d6) δ 8.19- | 3 |
| | | | | 8.11 (m, 2H), 7.73 (d, J = 1.6 Hz, 1H), | |
| | | | | 7.47 (d, J = 5.6 Hz, 1H), 7.34-7.24 | |
| | | | | (m, 2H), 4.16 (d, J = 7.0 Hz, 2H), 3.91 | |
| | | | | (q, J = 6.4 Hz, 2H), 3.70-3.40 (m, 8H), | |
| | | | | 3.22 (d, J = 9.5 Hz, 3H), 1.54 (s, 3H), | |
| | | | | 1.49 (s, 3H), 1.47 (s, 3H), 1.31-1.22 | |
| | | | | (m, 2H), 0.94-0.86 (m, 2H). | |
| 38 | 1.88 | 536.82 | C | ¹H NMR (400 MHz, DMSO-d6) δ 8.18- | 3 |
| | | | | 8.11 (m, 2H), 7.73 (d, J = 1.5 Hz, 1H), | |
| | | | | 7.47 (d, J = 5.8 Hz, 1H), 7.34-7.24 | |
| | | | | (m, 2H), 4.84 (dd, J = 73.6, 7.5 Hz, | |
| | | | | 1H), 4.43-4.29 (m, 1H), 3.98-3.35 | |
| | | | | (m, 6H), 1.54 (s, 3H), 1.53-1.44 (m, | |
| | | | | 7H), 1.31-1.25 (m, 2H), 0.98-0.87 | |
| | | | | (m, 11H). | |
| 39 | 1.74 | 548.44 | C | ¹H NMR (400 MHz, DMSO-d6) δ 8.21- | 3 |
| | | | | 8.07 (m, 2H), 7.74 (s, 1H), 7.47 (s, 1H), | |
| | | | | 7.34-7.23 (m, 2H), 7.20-7.02 (m, 1H), | |
| | | | | 4.23-3.39 (m, 6H), 1.61-1.42 (m, 12H), | |
| | | | | 1.32-1.20 (m, 2H), 0.96-0.86 (m, 2H). | |
| 40 | 3.85 | 548.44 | A | ¹H NMR (400 MHz, DMSO-d6) δ 8.21- | 3 |
| | | | | 8.10 (m, 2H), 7.91-7.81 (m, 1H), 7.78- | |
| | | | | 7.68 (m, 1H), 7.53-7.44 (m, 1H), | |
| | | | | 7.39-7.14 (m, 2H), 4.77 (dd, J = 7.5, | |
| | | | | 3.0 Hz, 1H), 4.14-3.39 (m, 7H), 1.73- | |
| | | | | 1.43 (m, 14H), 1.31-0.86 (m, 10H). | |
| 41 | 1.77 | 556.75 | C | ¹H NMR (400 MHz, DMSO-d6) δ 8.19- | 3 |
| | | | | 8.10 (m, 2H), 7.73 (s, 1H), 7.46 (d, J = | |
| | | | | 1.4 Hz, 1H), 7.33-7.26 (m, 2H), 7.26- | |
| | | | | 7.10 (m, 5H), 5.22 (t, J = 7.8 Hz, 1H), | |
| | | | | 4.57-4.38 (m, 1H), 3.98-3.42 (m, 6H), | |
| | | | | 2.99-2.67 (m, 2H), 1.54 (d, J = 3.9 Hz, | |
| | | | | 3H), 1.51-1.33 (m, 7H), 1.26 (dt, J = | |
| | | | | 5.9, 4.1 Hz, 2H), 0.94-0.87 (m, 2H). | |
| 42 | 1.93 | 518.81 | C | ¹H NMR (400 MHz, DMSO-d6) δ 8.21- | 3 |
| | | | | 8.10 (m, 2H), 7.74 (d, J = 5.9 Hz, 1H), | |
| | | | | 7.47 (d, J = 15.3 Hz, 1H), 7.35-7.24 | |
| | | | | (m, 2H), 3.93-3.42 (m, 6H), 1.77-1.52 | |
| | | | | (m, 8H), 1.50 (s, 3H), 1.43 (s, 3H), | |
| | | | | 1.39-1.07 (m, 7H), 0.95-0.86 (m, 2H). | |
| 43 | 2.07 | 532.81 | C | ¹H NMR (400 MHz, DMSO-d6) δ 8.19- | 3 |
| | | | | 8.11 (m, 2H), 7.73 (s, 1H), 7.46 (s, | |
| | | | | 1H), 7.32-7.24 (m, 2H), 3.96-3.42 | |
| | | | | (m, 6H), 2.10-1.98 (m, 2H), 1.54 (s, | |
| | | | | 3H), 1.53-1.19 (m, 14H), 1.17 (s, 3H), | |
| | | | | 0.93-0.87 (m, 2H). | |
| 44 | 4.21 | 492.44 | H | ¹H NMR (400 MHz, DMSO-d6) δ 8.18- | 3 |
| | | | | 8.10 (m, 2H), 7.73 (d, J = 4.1 Hz, 1H), | |
| | | | | 7.46 (d, J = 12.3 Hz, 1H), 7.34-7.25 | |
| | | | | (m, 2H), 4.01-3.43 (m, 6H), 2.85- | |
| | | | | 2.52 (m, 1H), 1.67-1.23 (m, 13H), | |
| | | | | 0.98 (dd, J = 7.9, 6.7 Hz, 3H), 0.94-0.87 | |
| | | | | (m, 2H), 0.80 (td, J = 7.4, 3.2 Hz, 3H). | |
| 45 | 3.98 | 490.43 | H | ¹H NMR (400 MHz, DMSO-d6) δ 8.19- | 3 |
| | | | | 8.09 (m, 2H), 7.74 (s, 1H), 7.47 (s, | |
| | | | | 1H), 7.34-7.24 (m, 2H), 3.98-3.41 | |
| | | | | (m, 6H), 1.55 (s, 3H), 1.49 (s, 6H), | |
| | | | | 1.32-1.22 (m, 5H), 0.93-0.87 (m, 2H), | |
| | | | | 0.85-0.77 (m, 2H), 0.60-0.51 (m, 2H). | |
| 46 | 4.00 | 478.43 | H | ¹H NMR (400 MHz, DMSO-d6) δ 8.18- | 3 |
| | | | | 8.11 (m, 2H), 7.74 (d, J = 4.1 Hz, 1H), | |
| | | | | 7.47 (d, J = 12.6 Hz, 1H), 7.34-7.24 | |
| | | | | (m, 2H), 3.96-3.44 (m, 6H), 2.84 (dt, | |
| | | | | J = 58.6, 6.7 Hz, 1H), 1.54 (s, 3H), | |
| | | | | 1.50 (s, 3H), 1.45 (s, 3H), 1.29-1.24 | |
| | | | | (m, 2H), 1.00 (dd, J = 6.6, 5.7 Hz, 7H), | |
| | | | | 0.94-0.87 (m, 2H). | |
| 47 | 2.00 | 554.84 | C | ¹H NMR (400 MHz, DMSO-d6) δ 8.19- | 3 |
| | | | | 8.09 (m, 2H), 7.72 (d, J = 2.3 Hz, 1H), | |
| | | | | 7.41 (d, J = 11.5 Hz, 1H), 7.37-7.24 | |
| | | | | (m, 6H), 7.23-7.16 (m, 1H), 3.97- | |
| | | | | 3.42 (m, 6H), 2.02-1.89 (m, 1H), 1.64- | |
| | | | | 1.41 (m, 7H), 1.25 (d, J = 5.4 Hz, 3H), | |
| | | | | 1.13 (s, 1H), 0.96-0.85 (m, 2H), 0.79 | |
| | | | | (td, J = 7.3, 1.8 Hz, 3H). | |
| 48 | 4.15 | 490.43 | H | ¹H NMR (400 MHz, DMSO-d6) δ 8.20- | 3 |
| | | | | 8.10 (m, 2H), 7.73 (d, J = 2.8 Hz, 1H), | |
| | | | | 7.46 (d, J = 8.7 Hz, 1H), 7.34-7.25 (m, | |
| | | | | 2H), 3.98-3.36 (m, 6H), 2.27-2.02 (m, | |
| | | | | 5H), 1.99-1.64 (m, 2H), 1.54 (d, J = | |
| | | | | 2.0 Hz, 3H), 1.52-1.42 (m, 6H), | |
| | | | | 1.32-1.22 (m, 2H), 0.99-0.84 (m, 2H). | |
| 49 | 3.91 | 476.42 | H | ¹H NMR (400 MHz, DMSO-d6) δ 8.20- | 3 |
| | | | | 8.20-8.09 (m, 2H), 7.73 (d, J = 2.3 Hz, | |
| | | | | 1H), 7.47 (d, J = 11.3 Hz, 1H), 7.34- | |
| | | | | 7.21 (m, 2H), 4.05-3.47 (m, 6H), 2.14- | |
| | | | | 1.75 (m, 1H), 1.60-1.49 (m, 6H), 1.45 | |
| | | | | (s, 3H), 1.32-1.22 (m, 2H), 0.94-0.86 | |
| | | | | (m, 2H), 0.80-0.67 (m, 4H). | |
| 50 | 4.83 | 532.39 | H | ¹H NMR (400 MHz, DMSO-d6) δ 8.19- | 3 |
| | | | | 8.19-8.09 (m, 2H), 7.73 (d, J = 2.2 Hz, | |
| | | | | 1H), 7.46 (d, J = 5.0 Hz, 1H), 7.34- | |
| | | | | 7.23 (m, 2H), 4.05-3.43 (m, 6H), 1.57- | |
| | | | | 1.52 (m, 3H), 1.52-1.42 (m, 6H), 1.30- | |
| | | | | 1.24 (m, 2H), 1.19-1.07 (m, 12H), | |
| | | | | 0.94-0.87 (m, 2H). | |
| 51 | 3.93 | 501.41 | H | ¹H NMR (400 MHz, DMSO-d6) δ 8.19- | 3 |
| | | | | 8.11 (m, 2H), 7.74 (s, 1H), 7.49 (s, | |
| | | | | 1H), 7.35-7.24 (m, 2H), 4.08-3.44 | |
| | | | | (m, 6H), 1.70-1.42 (m, 13H), 1.31- | |
| | | | | 1.24 (m, 2H), 0.94-0.84 (m, 2H). | |
| 52 | 1.92 | 506.49 | C | ¹H NMR (400 MHz, DMSO-d6) δ 8.15- | 3 |
| | | | | (dt, J = 8.6, 5.6 Hz, 2H), 7.73 (s, 1H), | |
| | | | | 7.46 (s, 1H), 7.29 (td, J = 8.8, 3.9 Hz, | |
| | | | | 2H), 3.92-3.60 (m, 7H), 1.61 (q, J = | |
| | | | | 7.3 Hz, 2H), 1.57-1.46 (m, 9H), 1.33 - | |
| | | | | 1.24 (m, 2H), 1.16 (S, 6H), 0.96-0.87 | |
| | | | | (m, 2H), 0.79 (t, J = 7.4 Hz, 3H). | |
| 53 | 4.72 | 586.37 | H | ¹H NMR (400 MHz, DMSO-d6) δ 8.19- | 3 |
| | | | | 8.09 (m, 2H), 7.71 (s, 1H), 7.46-7.11 | |
| | | | | (m, 7H), 3.99-3.40 (m, 6H), 1.65- | |
| | | | | 1.14 (m, 15H), 0.93-0.81 (m, 2H). | |
| 54 | 3.68 | 520.46 | H | ¹H NMR (400 MHz, DMSO-d6) δ 8.23- | 3 |
| | | | | 8.06 (m, 2H), 7.74 (d, J = 5.1 Hz, 1H), | |
| | | | | 7.47 (d, J = 16.1 Hz, 1H), 7.34-7.22 | |
| | | | | (m, 2H), 4.00-3.34 (m, 9H), 1.67- | |
| | | | | 1.46 (m, 10H), 1.44 (s, 3H), 1.31-1.24 | |
| | | | | (m, 2H), 0.95-0.83 (m, 2H). | |
| 55 | 4.27 | 544.44 | H | ¹H NMR (400 MHz, DMSO-d6) δ 8.21- | 3 |
| | | | | 8.09 (m, 2H), 7.74 (s, 1H), 7.47 (s, | |
| | | | | 1H), 7.35-7.23 (m, 2H), 4.03-3.41 | |
| | | | | (m, 6H), 1.59-1.40 (m, 9H), 1.37- | |
| | | | | 1.15 (m, 6H), 0.94-0.86 (m, 2H). | |
| 56 | 3.83 | 506.42 | H | ¹H NMR (400 MHz, DMSO-d6) δ 8.27- | 3 |
| | | | | 8.07 (m, 2H), 7.73 (d, J = 1.0 Hz, 1H), | |
| | | | | 7.46 (d, J = 3.2 Hz, 1H), 7.38-7.22 | |
| | | | | (m, 2H), 6.02-5.87 (m, 1H), 3.98- | |
| | | | | 3.43 (m, 6H), 2.58-2.50 (m, 2H), 2.08- | |
| | | | | 1.95 (m, 2H), 1.58-1.52 (m, 3H), | |
| | | | | 1.51-1.41 (m, 6H), 1.30-1.22 (m, | |
| | | | | 2H), 0.95-0.85 (m, 2H). | |
| 57 | 3.54 | 536.45 | H | ¹H NMR (400 MHz, DMSO-d6) δ 8.21- | 3 |
| | | | | 8.05 (m, 2H), 7.73 (s, 1H), 7.47 (s, | |
| | | | | 1H), 7.38-7.17 (m, 2H), 5.58 (s, 1H), | |
| | | | | 4.19-3.39 (m, 8H), 2.05-1.86 (m, | |
| | | | | 2H), 1.68-1.41 (m, 11H), 1.30-1.22 | |
| | | | | (m, 2H), 0.93-0.87 (m, 2H). | |
| 58 | 3.96 | 574.44 | H | ¹H NMR (400 MHz, DMSO-d6) δ 8.20- | 3 |
| | | | | 8.06 (m, 2H), 7.74 (d, J = 5.4 Hz, 1H), | |
| | | | | 7.47 (d, J = 15.5 Hz, 1H), 7.35-7.22 | |
| | | | | (m, 2H), 6.59 (d, J = 26.1 Hz, 1H), 3.88 | |
| | | | | (s, 2H), 3.68-3.56 (m, 2H), 3.54-3.46 | |
| | | | | (m, 2H), 3.07-2.88 (m, 1H), 2.60 (dt, | |
| | | | | J = 20.2, 11.0 Hz, 2H), 2.38 (t, J = 11.1 | |
| | | | | Hz, 2H), 1.56-1.51 (m, 3H), 1.48 (s, | |
| | | | | 3H), 1.45 (s, 3H), 1.30-1.23 (m, 2H), | |
| | | | | 0.95-0.83 (m, 2H). | |
| 59 | 1.61 | 494.69 | C | ¹H NMR (400 MHz, DMSO-d6) δ 8.20- | 3 |
| | | | | 8.07 (m, 2H), 7.74 (d, J = 2.8 Hz, 1H), | |
| | | | | 7.47 (d, J = 9.6 Hz, 1H), 7.39-7.21 | |
| | | | | (m, 2H), 4.87 (s, 1H), 4.39-4.13 (m, | |
| | | | | 1H), 3.91 (q, J = 5.2 Hz, 2H), 3.76- | |
| | | | | 3.40 (m, 4H), 1.71-1.57 (m, 1H), 1.54 | |
| | | | | (s, 3H), 1.52-1.42 (m, 7H), 1.30-1.23 | |
| | | | | (m, 2H), 0.94-0.89 (m, 2H), 0.86 (t, | |
| | | | | J = 7.4 Hz, 3H). | |
| 60 | 1.55 | 494.41 | C | ¹H NMR (400 MHz, DMSO-d6) δ 8.23- | 3 |
| | | | | 8.14 (m, 2H), 7.90-7.86 (m, 1H), 7.56- | |
| | | | | 7.47 (m, 1H), 7.38-7.30 (m, 2H), 3.99- | |
| | | | | 3.90 (m, 3H), 3.82-3.64 (m, 6H), 3.54- | |
| | | | | 3.48 (m, 1H), 3.47-3.43 (m, 1H), 3.35- | |
| | | | | 3.27 (m, 1H), 2.16-1.91 (m, 2H), 1.55- | |
| | | | | 1.51 (m, 3H), 1.50-1.47 (m, 3H), 1.44- | |
| | | | | 1.40 (m, 6H). | |
| 61 | 1.58 | 482.69 | C | ¹H NMR (400 MHz, DMSO-d6) δ 8.22- | 3 |
| | | | | 8.16 (m, 2H), 7.90-7.86 (m, 1H), 7.54- | |
| | | | | 7.49 (m, 1H), 7.38-7.29 (m, 2H), | |
| | | | | 4.32-4.14 (m, 1H), 4.03-3.42 (m, | |
| | | | | 5H), 1.71-1.59 (m, 1H), 1.57-1.46 | |
| | | | | (m, 7H), 1.45-1.37 (m, 6H), 0.94- | |
| | | | | 0.84 (m, 3H). | |
| 62 | 1.59 | 496.46 | C | ¹H NMR (400 MHz, DMSO-d6) δ 8.22- | 3 |
| | | | | 8.15 (m, 2H), 7.89-7.86 (m, 1H), 7.54- | |
| | | | | 7.48 (m, 1H), 7.38-7.28 (m, 2H), | |
| | | | | 4.00-3.89 (m, 2H), 3.77 (t, J = 5.4 Hz, | |
| | | | | 1H), 3.70 (d, J = 13.6 Hz, 2H), 3.58- | |
| | | | | 3.51 (m, 1H), 3.50-3.39 (m, 1H), 2.44 | |
| | | | | (s, 1H), 1.55-1.47 (m, 6H), 1.45-1.38 | |
| | | | | (m, 6H), 1.23-1.17 (m, 6H). | |
| 63 | 1.58 | 482.41 | C | ¹H NMR (400 MHz, DMSO-d6) δ 8.21- | 3 |
| | | | | 8.11 (m, 2H), 7.88-7.82 (m, 1H), 7.52- | |
| | | | | 7.45 (m, 1H), 7.37-7.24 (m, 2H), | |
| | | | | 4.22 (dd, J = 36.8, 6.6 Hz, 1H), 3.98- | |
| | | | | 3.41 (m, 7H), 3.18 (d, J = 4.0 Hz, 3H), | |
| | | | | 1.51 (d, J = 1.5 Hz, 3H), 1.46 (s, 3H), | |
| | | | | 1.41-1.36 (m, 6H), 1.24-1.16 (m, 3H). | |
| 64 | 1.71 | 508.45 | C | ¹H NMR (400 MHz, DMSO-d6) δ 8.18 | 3 |
| | | | | (s, 2H), 7.90-7.86 (m, 1H), 7.54-7.49 | |
| | | | | (m, 1H), 7.38-7.30 (m, 2H), 4.22- | |
| | | | | 4.11 (m, 1H), 4.01-3.39 (m, 9H), 1.83 | |
| | | | | (d, J = 9.7 Hz, 1H), 1.69-1.46 (m, | |
| | | | | 11H), 1.42 (dd, J = 6.9, 3.6 Hz, 6H). | |
| 65 | 1.48 | 490.39 | C | ¹H NMR (400 MHz, DMSO-d6) δ 8.24- | 3 |
| | | | | 8.14 (m, 2H), 7.91-7.86 (m, 1H), 7.84- | |
| | | | | 7.77 (m, 1H), 7.54-7.49 (m, 1H), | |
| | | | | 7.37-7.29 (m, 2H), 6.71-6.63 (m, 1H), | |
| | | | | 4.24 (s, 1H), 4.13-3.92 (m, 3H), 3.85- | |
| | | | | 3.79 (m, 1H), 3.51-3.33 (m, 1H), 1.56 | |
| | | | | (s, 3H), 1.48 (s, 3H), 1.45-1.37 (m, 6H). | |
| 66 | 1.79 | 544.43 | C | ¹H NMR (400 MHz, DMSO-d6) δ 8.22- | 3 |
| | | | | 8.14 (m, 2H), 7.89-7.86 (m, 1H), 7.49- | |
| | | | | 7.28 (m, 8H), 5.31-5.11 (m, 1H), 3.98- | |
| | | | | 3.54 (m, 5H), 3.52-3.37 (m, 1H), 3.33- | |
| | | | | 3.29 (m, 3H), 1.52-1.36 (m, 9H), 1.32 | |
| | | | | (d, J = 4.9 Hz, 3H). | |
| 67 | 1.86 | 544.43 | C | ¹H NMR (400 MHz, DMSO-d6) δ 8.22- | 3 |
| | | | | 8.14 (m, 2H), 7.88 (s, 1H), 7.54-7.49 | |
| | | | | (m, 1H), 7.37-7.23 (m, 4H), 6.98- | |
| | | | | 6.91 (m, 1H), 6.90-6.84 (m, 2H), 5.38- | |
| | | | | 5.13 (m, 1H), 4.04-3.59 (m, 5H), | |
| | | | | 3.56-3.48 (m, 1H), 3.47-3.36 (m, | |
| | | | | 1H), 1.64-1.35 (m, 15H). | |
| 68 | 1.72 | 536.4 | C | ¹H NMR (400 MHz, DMSO-d6) δ 8.23- | 3 |
| | | | | 8.15 (m, 2H), 7.88 (s, 1H), 7.51 (s, | |
| | | | | 1H), 7.37-7.30 (m, 2H), 7.20-7.07 | |
| | | | | (m, 1H), 4.22-3.83 (m, 4H), 3.67 (s, | |
| | | | | 1H), 3.57-3.49 (m, 1H), 3.47-3.38 | |
| | | | | (m, 1H), 1.61-1.46 (m, 9H), 1.46- | |
| | | | | 1.37 (m, 6H). | |
| 69 | 1.77 | 510.46 | C | ¹H NMR (400 MHz, DMSO-d6) δ 8.20- | 3 |
| | | | | 8.09 (m, 2H), 7.85 (s, 1H), 7.47 (s, | |
| | | | | 1H), 7.37-7.24 (m, 2H), 4.98-4.80 | |
| | | | | (m, 1H), 4.19-4.02 (m, 2H), 4.00- | |
| | | | | 3.75 (m, 2H), 3.65 (s, 1H), 3.58-3.45 | |
| | | | | (m, 1H), 3.44-3.36 (m, 1H), 1.83- | |
| | | | | 1.69 (m, 2H), 1.60-1.45 (m, 8H), 1.39 | |
| | | | | (d, J = 6.9 Hz, 6H), 0.87-0.69 (m, 6H). | |
| 70 | 1.52 | 480.36 | C | ¹H NMR (400 MHz, DMSO-d6) δ 8.21- | 3 |
| | | | | 8.15 (m, 2H), 7.88 (s, 1H), 7.51 (s, | |
| | | | | 1H), 7.38-7.30 (m, 2H), 4.08-3.88 (m, | |
| | | | | 5H), 3.49-3.38 (m, 1H), 1.52 (s, 7H), | |
| | | | | 1.45-1.39 (m, 6H), 1.01-0.92 (m, 2H), | |
| | | | | 0.87-0.77 (m, 2H). | |
| 71 | 1.84 | 492.12 | C | ¹H NMR (400 MHz, DMSO-d6) δ 8.19- | 3 |
| | | | | 8.12 (m, 2H), 7.86-7.84 (m, 1H), 7.51- | |
| | | | | 7.45 (m, 1H), 7.34-7.26 (m, 2H), 3.95- | |
| | | | | 3.85 (m, 2H), 3.73 (t, J = 5.5 Hz, 1H), | |
| | | | | 3.67 (s, 1H), 3.62 (s, 1H), 3.50-3.45 | |
| | | | | (m, 1H), 3.44-3.34 (m, 1H), 3.05-2.86 | |
| | | | | (m, 1H), 1.88-1.69 (m, 3H), 1.69-1.56 | |
| | | | | (m, 4H), 1.55-1.47 (m, 4H), 1.46-1.43 | |
| | | | | (m, 3H), 1.42-1.37 (m, 6H). | |
| 72 | 1.72 | 500.66 | C | ¹H NMR (400 MHz, DMSO-d6) δ 8.21- | 3 |
| | | | | 8.15 (m, 2H), 7.89-7.87 (m, 1H), 7.54- | |
| | | | | 7.50 (m, 1H), 7.36-7.30 (m, 2H), 4.06- | |
| | | | | 3.92 (m, 3H), 3.89-3.48 (m, 3H), 3.49- | |
| | | | | 3.38 (m, 1H), 3.30-3.02 (m, 1H), 2.05- | |
| | | | | 1.81 (m, 2H), 1.60-1.45 (m, 6H), 1.45- | |
| | | | | 1.38 (m, 6H). | |
| 73 | 1.68 | 491.33 | C | ¹H NMR (400 MHz, DMSO-d6) δ 9.16- | 3 |
| | | | | 9.11 (m, 1H), 8.23-8.15 (m, 2H), 7.91- | |
| | | | | 7.85 (m, 1H), 7.56-7.49 (m, 1H), | |
| | | | | 7.38-7.29 (m, 2H), 6.95-6.87 (m, | |
| | | | | 1H), 4.09-4.01 (m, 1H), 3.97-3.91 | |
| | | | | (m, 1H), 3.89-3.80 (m, 3H), 3.78- | |
| | | | | 3.70 (m, 1H), 3.47-3.33 (m, 1H), 1.59- | |
| | | | | 1.47 (m, 6H), 1.45-1.34 (m, 6H). | |
| 74 | 1.69 | 496.41 | C | ¹H NMR (400 MHz, DMSO-d6) δ 8.22- | 3 |
| | | | | 8.15 (m, 2H), 7.90-7.87 (m, 1H), 7.55- | |
| | | | | 7.49 (m, 1H), 7.38-7.30 (m, 2H), | |
| | | | | 4.08 (d, J = 6.1 Hz, 1H), 4.03-3.91 | |
| | | | | (m, 2H), 3.88-3.27 (m, 6H), 2.02- | |
| | | | | 1.79 (m, 1H), 1.58-1.46 (m, 6H), 1.46- | |
| | | | | 1.37 (m, 6H), 0.93-0.80 (m, 6H). | |
| 75 | 1.60 | 494.41 | C | ¹H NMR (400 MHz, DMSO-d6) δ 8.22- | 3 |
| | | | | 8.15 (m, 2H), 7.89-7.87 (m, 1H), 7.53- | |
| | | | | 7.49 (m, 1H). 7.37-7.29 (m, 2H), | |
| | | | | 4.75-4.64 (m, 1H), 3.99-3.84 (m, | |
| | | | | 3H), 3.84-3.69 (m, 4H), 3.66-3.61 | |
| | | | | (m, 1H), 3.50 (t, J = 5.9 Hz, 1H), 3.47- | |
| | | | | 3.40 (m, 1H), 2.11-1.98 (m, 2H), 1.94- | |
| | | | | 1.77 (m, 1H), 1.57-1.51 (m, 3H), | |
| | | | | 1.49 (s, 3H), 1.45-1.39 (m, 6H). | |
| 76 | 1.67 | 486.75 | C | ¹H NMR (400 MHz, DMSO-d6) δ 8.24- | 3 |
| | | | | 8.13 (m, 2H), 7.90 (s, 1H), 7.63 (s, | |
| | | | | 1H), 7.43-7.27 (m, 2H), 4.78-3.40 | |
| | | | | (m, 4H), 3.23-3.00 (m, 2H), 2.07- | |
| | | | | 1.77 (m, 2H), 1.48-1.39 (m, 6H), 1.34- | |
| | | | | 1.08 (m, 4H). | |
| 77 | 1.89 | 468.37 | C | ¹H NMR (400 MHz, Chloroform-d) δ | 1 |
| | | | | 8.00-7.94 (m, 2H), 7.54 (s, 1H), 7.47 | |
| | | | | (s, 1H), 7.20-7.13 (m, 2H), 3.87-3.75 | |
| | | | | (m, 4H), 3.62-3.53 (m, 4H), 3.49- | |
| | | | | 3.38 (m, 1H), 1.49 (s, 9H), 1.45 (d, J = | |
| | | | | 6.9 Hz, 6H). | |
| 78 | 1.95 | 482.09 | C | ¹H NMR (400 MHz, Chloroform-d) δ | 1 |
| | | | | 8.03-7.93 (m, 2H), 7.54 (s, 1H), 7.45 | |
| | | | | (s, 1H), 7.23-7.13 (m, 2H), 5.00-3.85 | |
| | | | | (m, 4H), 3.57-2.87 (m, 4H), 1.58- | |
| | | | | 1.35 (m, 18H). | |
| 79 | 1.95 | 482.13 | C | ¹H NMR (400 MHz, Chloroform-d) δ | 1 |
| | | | | 8.07-7.92 (m, 2H), 7.54 (s, 1H), 7.45 | |
| | | | | (s, 1H), 7.23-7.10 (m, 2H), 4.84-3.86 | |
| | | | | (m, 4H), 3.55-2.86 (m, 4H), 1.77- | |
| | | | | 1.18 (m, 18H). | |
| 80 | 1.49 | 480.03 | C | ¹H NMR (400 MHz, DMSO-d6) δ 8.24- | 3 |
| | | | | 8.16 (m, 2H), 7.90 (s, 1H), 7.69-7.53 | |
| | | | | (m, 1H), 7.38-7.30 (m, 2H), 4.66 (d, | |
| | | | | J = 15.6 Hz, 1H), 4.52-3.59 (m, 8H), | |
| | | | | 3.57-2.77 (m, 4H), 2.21-1.84 (m, 2H), | |
| | | | | 1.47-1.38 (m, 6H), 1.30-1.03 (m, 3H). | |
| 81 | 1.79 | 478.12 | C | ¹H NMR (400 MHz, Chloroform-d) δ | 3 |
| | | | | 8.25-8.13 (m, 2H), 7.90 (s, 1H), 7.74- | |
| | | | | 7.52 (m, 1H), 7.42-7.28 (m, 2H), 4.80- | |
| | | | | 4.57 (m, 1H), 4.51-3.81 (m, 3H), | |
| | | | | 3.59-2.79 (m, 5H), 1.93-1.48 (m, 8H), | |
| | | | | 1.47-1.38 (m, 6H), 1.32-1.00 (m, 3H). | |
| 82 | 1.81 | 480.41 | C | ¹H NMR (400 MHz, DMSO-d6) δ 8.23- | 3 |
| | | | | 8.16 (m, 2H), 7.90 (s, 1H), 7.61 (s, | |
| | | | | 1H), 7.38-7.30 (m, 2H), 4.77 (s, 1H), | |
| | | | | 4.59-3.95 (m, 4H), 3.43 (h, J = 6.9 | |
| | | | | Hz, 1H), 3.08 (d, J = 59.0 Hz, 2H), | |
| | | | | 2.72-2.56 (m, 1H), 1.67-1.31 (m, 10H), | |
| | | | | 1.17 (dd, J = 47.4, 6.6 Hz, 3H), 0.93- | |
| | | | | 0.71 (m, 6H). | |
| 83 | 1.68 | 534.4 | C | ¹H NMR (400 MHz, DMSO-d6) δ 8.23- | 3 |
| | | | | 8.15 (m, 2H), 7.88 (s, 1H), 7.56 (s, | |
| | | | | 1H), 7.48-7.28 (m, 4H), 7.27-7.07 | |
| | | | | (m, 2H), 6.19-5.32 (m, 1H), 4.82- | |
| | | | | 4.56 (m, 1H), 4.53-3.79 (m, 4H), 3.72- | |
| | | | | 2.57 (m, 4H), 1.49-1.32 (m, 6H), | |
| | | | | 1.20-0.97 (m, 2H), 0.85-0.69 (m, 1H). | |
| 84 | 1.74 | 530.43 | C | ¹H NMR (400 MHz, DMSO-d6) δ 8.21- | 3 |
| | | | | 8.14 (m, 2H), 7.88 (s, 1H), 7.61-7.51 | |
| | | | | (m, 1H), 7.44-7.28 (m, 7H), 5.27- | |
| | | | | 5.17 (m, 1H), 4.78-4.62 (m, 1H), 4.55- | |
| | | | | 3.80 (m, 2H), 3.48-3.27 (m, 6H), | |
| | | | | 3.24-2.95 (m, 1H), 2.93-2.74 (m, 1H), | |
| | | | | 1.47-1.34 (m, 6H), 1.08-0.95 (m, 3H). | |
| 85 | 1.81 | 530.8 | C | ¹H NMR (400 MHz, DMSO-d6) δ 8.27- | 3 |
| | | | | 8.14 (m, 2H), 7.90 (s, 1H), 7.71-7.52 | |
| | | | | (m, 1H), 7.41-7.20 (m, 4H), 7.03- | |
| | | | | 6.77 (m, 3H), 5.37-5.16 (m, 1H), 4.73- | |
| | | | | 4.51 (m, 1H), 4.50-3.88 (m, 3H), | |
| | | | | 3.67-2.91 (m, 5H), 1.55-1.36 (m, | |
| | | | | 8H), 1.32-0.99 (m, 3H). | |
| 86 | 1.59 | 477.33 | C | ¹H NMR (400 MHz, DMSO-d6) δ 8.75 | 3 |
| | | | | (d, J = 1.9 Hz, 1H), 8.19-8.12 (m, 2H), | |
| | | | | 7.87 (s, 1H), 7.59 (s, 1H), 7.35-7.25 | |
| | | | | (m, 2H), 6.96 (d, J = 1.9 Hz, 1H), 4.31 | |
| | | | | (d, J = 43.9 Hz, 3H), 3.97-2.97 (m, | |
| | | | | 5H), 1.46-1.13 (m, 9H). | |
| 87 | 1.51 | 468.37 | C | ¹H NMR (400 MHz, DMSO-d6) δ | 3 |
| | | | | 8.16 (m, 2H), 7.90 (s, 1H), 7.68-7.51 | |
| | | | | (m, 1H), 7.39-7.29 (m, 2H), 5.37 (d, | |
| | | | | J = 104.7 Hz, 1H), 4.73 (d, J = 75.6 Hz, | |
| | | | | 1H), 4.22 (s, 2H), 3.54 (s, 5H), 1.58- | |
| | | | | 1.02 (m, 15H). | |
| 88 | 1.65 | 494.41 | C | ¹H NMR (400 MHz, DMSO-d6) δ 8.22- | 3 |
| | | | | 8.16 (m, 2H), 7.90 (s, 1H), 7.67-7.52 | |
| | | | | (m, 1H), 7.38-7.30 (m, 2H), 4.62 (s, | |
| | | | | 1H), 4.52-3.80 (m, 6H), 3.58-2.87 | |
| | | | | (m, 3H), 1.89-1.75 (m, 1H), 1.71- | |
| | | | | 1.38 (m, 12H), 1.30- 1.04 (m, 3H). | |
| 89 | 1.55 | 480.36 | C | ¹H NMR (400 MHz, DMSO-d6) δ 8.23- | 3 |
| | | | | 8.16 (m, 2H), 7.89 (s, 1H), 7.69-7.50 | |
| | | | | (m, 1H), 7.39-7.26 (m, 2H). 6.19- | |
| | | | | 5.84 (m, 1H), 4.74-3.96 (m, 4H), 3.89- | |
| | | | | 2.73 (m, 5H), 2.71-2.55 (m, 1H), 2.14- | |
| | | | | 1.95 (m, 2H), 1.86-1.68 (m, 1H), 1.57- | |
| | | | | 1.35 (m, 7H), 1.29-1.07 (m, 3H). | |
| 90 | 1.82 | 510.13 | C | ¹H NMR (400 MHz, DMSO-d6) δ 8.27- | 3 |
| | | | | 8.15 (m, 2H), 7.90 (s, 1H), 7.75-7.52 | |
| | | | | (m, 1H), 7.40-7.29 (m, 2H), 5.55- | |
| | | | | 4.90 (m, 1H), 4.73 (s, 1H), 4.53-3.81 | |
| | | | | (m, 4H), 3.61-3.39 (m, 1H), 3.27- | |
| | | | | 2.91 (m, 2H), 1.51-1.38 (m, 6H), 1.36- | |
| | | | | 1.04 (m, 4H), 1.01-0.82 (m, 11H). | |
| 91 | 1.45 | 510.74 | C | ¹H NMR (400 MHz, DMSO-d6) δ 8.23- | 3 |
| | | | | 8.15 (m, 2H), 7.90 (S, 1H), 7.69-7.53 | |
| | | | | (m, 1H), 7.38-7.30 (m, 2H), 5.88- | |
| | | | | 5.55 (m, 1H), 5.40-4.57 (m, 2H), 4.54- | |
| | | | | 3.84 (m, 6H), 3.51-2.77 (m, 3H), | |
| | | | | 2.13-1.79 (m, 2H), 1.72-1.51 (m, 2H), | |
| | | | | 1.48-1.35 (m, 6H), 1.32-1.05 (m, 4H). | |
| 92 | 1.49 | 466.69 | C | ¹H NMR (400 MHz, DMSO-d6) δ 8.22- | 3 |
| | | | | 8.16 (m, 2H), 7.89 (s, 1H), 7.59 (s, | |
| | | | | 1H), 7.38-7.29 (m, 2H), 3.87-3.56 | |
| | | | | (m, 6H), 3.49-3.39 (m, 1H), 2.63- | |
| | | | | 2.52 (m, 3H), 2.11-1.98 (m, 3H), 1.81- | |
| | | | | 1.67 (m, 1H), 1.52-1.38 (m, 8H). | |
| 93 | 3.22 | 440.34 | H | ¹H NMR (400 MHz, DMSO-d6) δ 8.25- | 3 |
| | | | | 8.16 (m, 2H), 7.90 (s, 1H), 7.60 (s, | |
| | | | | 1H), 7.38-7.29 (m, 2H), 4.54-4.41 | |
| | | | | (m, 1H), 4.41-3.50 (m, 8H), 3.49- | |
| | | | | 3.36 (m, 1H), 1.43 (d, J = 6.9 Hz, 6H), | |
| | | | | 1.27-1.18 (m, 4H). | |
| 94 | 1.75 | 496.09 | C | ¹H NMR (400 MHz, DMSO-d6) δ 8.23- | 3 |
| | | | | 8.16 (m, 2H), 7.90 (s, 1H), 7.61 (s, | |
| | | | | 1H), 7.37-7.30 (m, 2H), 4.90 (d, J = | |
| | | | | 7.2 Hz, 1H), 4.49-4.39 (m, 1H), 3.99- | |
| | | | | 3.58 (m, 8H), 3.57-3.37 (m, 2H), 1.60- | |
| | | | | 1.49 (m, 1H), 1.43 (d, J = 6.9 Hz, 6H), | |
| | | | | 0.95 (s, 9H). | |
| 95 | 1.39 | 496.37 | C | ¹H NMR (400 MHz, DMSO-d6) δ 8.23- | 3 |
| | | | | 8.14 (m, 2H), 7.90 (s, 1H), 7.60 (s, | |
| | | | | 1H), 7.39-7.28 (m, 2H), 5.85-5.61 | |
| | | | | (m, 1H), 4.87-4.47 (m, 2H), 4.24- | |
| | | | | 3.51 (m, 10H), 3.51-3.34 (m, 1H), | |
| | | | | 2.05-1.89 (m, 2H), 1.67-1.53 (m, | |
| | | | | 2H), 1.42 (d, J = 7.0 Hz, 6H). | |
| 96 | 1.45 | 454.32 | C | ¹H NMR (400 MHz, DMSO-d6) δ 8.22- | 3 |
| | | | | 8.15 (m, 2H), 7.90 (s, 1H), 7.60 (s, | |
| | | | | 1H), 7.37-7.30 (m, 2H), 4.23-3.50 | |
| | | | | (m, 9H), 3.49-3.39 (m, 1H), 1.42 (d, | |
| | | | | J = 6.9 Hz, 6H), 1.38-1.30 (m, 6H). | |
| 97 | 1.54 | 480.69 | C | ¹H NMR (400 MHz, DMSO-d6) δ 8.23- | 3 |
| | | | | 8.15 (m, 2H), 7.89 (s, 1H), 7.59-7.55 | |
| | | | | (m, 1H), 7.37-7.29 (m, 2H), 6.20- | |
| | | | | 5.91 (m, 1H), 4.84-4.46 (m, 1H), 4.41- | |
| | | | | 4.08 (m, 2H), 4.06-3.57 (m, 2H), 3.52- | |
| | | | | 3.27 (m, 1H), 3.24-2.56 (m, 2H), 2.18- | |
| | | | | 1.95 (m, 2H), 1.84-1.68 (m, 1H), 1.55- | |
| | | | | 1.37 (m, 8H), 1.36-1.11 (m, 4H). | |
| 98 | 1.42 | 454.04 | C | ¹H NMR (400 MHz, DMSO-d6) δ 8.24- | 3 |
| | | | | 8.16 (m, 2H), 7.90 (s, 1H), 7.58 (d, J = | |
| | | | | 2.9 Hz, 1H), 7.33 (t, J = 8.9 Hz, 2H), | |
| | | | | 4.47 (dd, J = 26.4, 6.4 Hz, 1H), 4.22 | |
| | | | | (d, J = 13.1 Hz, 1H), 3.98-3.74 (m, 1H), | |
| | | | | 3.43 (p, J = 6.9 Hz, 2H), 3.24 (s, 1H), | |
| | | | | 2.94 (s, 1H), 1.43 (d, J = 7.0 Hz, 6H), | |
| | | | | 1.24 (ddd, J = 32.0, 22.0, 11.8 Hz, 9H). | |
| 99 | 1.81 | 510.69 | C | ¹H NMR (400 MHz, DMSO-d6) δ 8.24- | 3 |
| | | | | 8.15 (m, 2H), 7.89 (s, 1H), 7.58 (s, | |
| | | | | 1H), 7.33 (t, J = 8.9 Hz, 2H), 5.11- | |
| | | | | 4.80 (m, 1H), 4.72-3.72 (m, 3H), | |
| | | | | 3.54-3.39 (m, 1H), 3.36-3.31 (m, 3H), | |
| | | | | 3.04-2.90 (m, 1H), 1.65-1.40 (m, 8H), | |
| | | | | 1.39-1.18 (m, 4H), 1.00-0.89 (m, 9H). | |
| 100 | 1.44 | 510.74 | C | ¹H NMR (400 MHz, DMSO-d6) δ 8.24- | 3 |
| | | | | 8.24-8.16 (m, 2H), 7.89 (s, 1H), 7.57 | |
| | | | | (s, 1H), 7.38-7.29 (m, 1H), 5.80-5.63 | |
| | | | | (m, 1H), 5.02-4.06 (m, 3H), 3.76-3.59 | |
| | | | | (m, 6H), 3.53-2.75 (m, 1H), 2.11-1.88 | |
| | | | | (m, 2H), 1.67-1.56 (m, 3H), 1.49-1.38 | |
| | | | | (m, 6H), 1.30-1.15 (m, 5H). | |
| 101 | 1.50 | 468.65 | C | ¹H NMR (400 MHz, DMSO-d6) δ 8.22- | 3 |
| | | | | 8.22-8.16 (m, 2H), 7.89 (s, 1H), 7.57 | |
| | | | | (s, 1H), 7.38-7.30 (m, 2H), 5.15-3.62 | |
| | | | | (m, 7H), 3.49-3.39 (m, 1H), 3.30-2.69 | |
| | | | | (m, 1H), 1.43 (d, J = 7.0 Hz, 6H), | |
| | | | | 1.40-1.19 (m, 9H). | |
| 102 | 1.55 | 480.69 | C | ¹H NMR (400 MHz, DMSO-d6) δ 8.23- | 3 |
| | | | | 8.15 (m, 2H), 7.89 (s, 1H), 7.59-7.55 | |
| | | | | (m, 1H), 7.38-7.29 (m, 2H), 4.86- | |
| | | | | 3.85 (m, 5H), 3.52-3.28 (m, 1H), 3.22- | |
| | | | | 3.05 (m, 1H), 2.96-2.87 (m, 1H), 2.73- | |
| | | | | 2.58 (m, 1H), 2.48-2.41 (m, 1H), 2.17- | |
| | | | | 1.92 (m, 2H), 1.84-1.68 (m, 1H), 1.54- | |
| | | | | 1.37 (m, 7H), 1.35-1.15 (m, 3H). | |
| 103 | 3.32 | 454.38 | H | ¹H NMR (400 MHz, DMSO-d6) δ 8.25- | 3 |
| | | | | 8.14 (m, 2H), 7.89 (s, 1H), 7.62-7.56 | |
| | | | | (m, 1H), 7.38-7.28 (m, 2H), 4.72- | |
| | | | | 3.52 (m, 3H), 3.50-3.07 (m, 2H), 3.01- | |
| | | | | 2.83 (m, 1H), 1.51-1.39 (m, 7H), | |
| | | | | 1.37-1.14 (m, 9H). | |
| 104 | 1.81 | 510.79 | C | ¹H NMR (400 MHz, DMSO-d6) δ 8.22- | 3 |
| | | | | 8.17 (m, 2H), 7.89 (s, 1H), 7.59-7.56 | |
| | | | | (m, 1H), 7.37-7.30 (m, 2H), 5.13- | |
| | | | | 4.87 (m, 1H), 4.76-4.44 (m, 2H), 4.41- | |
| | | | | 4.14 (m, 1H), 4.12-3.92 (m, 1H), | |
| | | | | 3.50-3.37 (m, 2H), 3.29-3.14 (m, | |
| | | | | 1H), 3.00-2.86 (m, 1H), 1.66-1.50 | |
| | | | | (m, 1H), 1.48-1.40 (m, 7H), 1.38- | |
| | | | | 1.15 (m, 4H), 1.01-0.90 (m, 9H). | |
| 105 | 1.44 | 510.41 | C | ¹H NMR (400 MHz, DMSO-d6) δ 8.23- | 3 |
| | | | | 8.16 (m, 2H), 7.89 (s, 1H), 7.57 (s, | |
| | | | | 1H), 7.40-7.29 (m, 2H), 5.84-5.62 | |
| | | | | (m, 1H), 5.05-3.84 (m, 6H), 3.78- | |
| | | | | 3.58 (m, 4H), 3.51-3.36 (m, 1H), 3.33- | |
| | | | | 2.78 (m, 1H), 2.07-1.86 (m, 2H), | |
| | | | | 1.70-1.55 (m, 2H), 1.42 (d, J = 6.9 | |
| | | | | Hz, 6H), 1.37-1.12 (m, 3H). | |
| 106 | 1.50 | 468.69 | C | ¹H NMR (400 MHz, DMSO-d6) δ 8.22- | 3 |
| | | | | 8.16 (m, 2H), 7.89 (s, 1H), 7.57 (s, | |
| | | | | 1H), 7.37-7.30 (m, 2H), 5.61-3.93 | |
| | | | | (m, 6H), 3.66-2.76 (m, 3H), 1.42 (d, | |
| | | | | J = 6.9 Hz, 6H), 1.39-1.12 (m, 9H). | |
| 107 | 4.35 | 514.38 | H | ¹H NMR (400 MHz, DMSO-d6) δ 8.20- | 12 |
| | | | | 8.09 (m, 2H), 7.74 (s, 1H), 7.49 (s, | |
| | | | | 1H), 7.36-7.23 (m, 2H), 3.79 (t, J = | |
| | | | | 5.4 Hz, 2H), 3.48-3.41 (m, 2H), 3.27- | |
| | | | | 3.23 (m, 2H), 3.14-3.06 (m, 2H), 1.75- | |
| | | | | 1.61 (m, 2H), 1.54 (s, 3H), 1.49 (s, | |
| | | | | 6H), 1.30-1.22 (m, 2H), 0.97 (t, J = | |
| | | | | 7.4 Hz, 3H), 0.93-0.86 (m, 2H). | |
| 108 | 3.70 | 480.68 | A | ¹H NMR (400 MHz, DMSO-d6) δ 8.21- | N/A |
| | | | | 8.07 (m, 2H), 7.73 (s, 1H), 7.46 (s, | |
| | | | | 1H), 7.34-7.23 (m, 2H), 4.06 (q, J = | |
| | | | | 7.1 Hz, 2H), 3.85 (s, 2H), 3.52 (s, 2H), | |
| | | | | 1.54 (s, 3H), 1.47 (s, 6H), 1.31-1.21 | |
| | | | | (m, 2H), 1.17 (t, J = 7.1 Hz, 3H), 0.93- | |
| | | | | 0.84 (m, 2H). | |
| 109 | 3.56 | 480.38 | H | ¹H NMR (400 MHz, Chloroform-d) δ | 3 |
| | | | | 8.00-7.90 (m, 2H), 7.58 (s, 1H), 7.55 | |
| | | | | (s, 1H), 7.23-7.12 (m, 2H), 5.13-2.75 | |
| | | | | (m, 5H), 1.78-1.55 (m, 9H), 1.47- | |
| | | | | 1.13 (m, 8H), 0.97 (s, 2H). | |
| 110 | 3.60 | 480.38 | H | ¹H NMR (400 MHz, Chloroform-d) δ | 3 |
| | | | | 8.01-7.90 (m, 2H), 7.58 (s, 1H), 7.56 | |
| | | | | (s, 1H), 7.22-7.11 (m, 2H), 5.01-3.11 | |
| | | | | (m, 8H), 1.79-1.43 (m, 6H), 1.41- | |
| | | | | 1.15 (m, 5H), 1.08-0.88 (m, 5H). | |
| 111 | 3.63 | 492.38 | H | ¹H NMR (400 MHz, Chloroform-d) δ | 3 |
| | | | | 7.99-7.92 (m, 2H), 7.58 (s, 1H), 7.57- | |
| | | | | 7.55 (m, 1H), 7.21-7.13 (m, 2H), 5.05- | |
| | | | | 2.73 (m, 8H), 1.77-1.46 (m, 6H), | |
| | | | | 1.45-0.90 (m, 6H), 0.63-0.39 (m, 4H). | |
| 112 | 3.86 | 494.36 | H | ¹H NMR (400 MHz, Chloroform-d) δ | 3 |
| | | | | 8.01-7.90 (m, 2H), 7.57 (s, 1H), 7.55 | |
| | | | | (s, 1H), 7.20-7.11 (m, 2H), 5.13-3.37 | |
| | | | | (m, 8H), 1.64-1.46 (m, 6H), 1.42- | |
| | | | | 1.02 (m, 7H), 0.94 (s, 2H), 0.90-0.74 | |
| | | | | (m, 3H). | |
| 113 | 4.04 | 508.4 | H | ¹H NMR (400 MHz, DMSO-d6) δ 8.21- | 3 |
| | | | | 8.10 (m, 2H), 7.76 (s, 1H), 7.56 (s, | |
| | | | | 1H), 7.35-7.22 (m, 2H), 5.20-3.44 | |
| | | | | (m, 8H), 1.85-1.65 (m, 1H), 1.62- | |
| | | | | 1.51 (m, 3H), 1.38-0.75 (m, 16H). | |
| 114 | 3.97 | 546.36 | H | ¹H NMR (400 MHz, DMSO-d6) δ 8.23- | 3 |
| | | | | 7.97 (m, 2H), 7.74 (s, 1H), 7.56-7.02 | |
| | | | | (m, 7H), 4.89-3.72 (m, 4H), 3.28- | |
| | | | | 3.17 (m, 2H), 1.66-0.65 (m, 12H). | |
| 115 | 4.00 | 476.39 | H | ¹H NMR (400 MHz, DMSO-d6) δ 8.21- | 3 |
| | | | | 8.06 (m, 2H), 7.75 (s, 1H), 7.54 (s, 1H), | |
| | | | | 7.35-7.22 (m, 2H), 4.87-2.77 (m, 6H), | |
| | | | | 2.23-1.63 (m, 7H), 1.54 (s, 3H), 1.35- | |
| | | | | 0.85 (m, 8H). | |
| 116 | 3.77 | 462.35 | H | ¹H NMR (400 MHz, DMSO-d6) δ 8.27- | 3 |
| | | | | 8.03 (m, 2H), 7.76 (s, 1H), 7.67-7.44 | |
| | | | | (m, 1H), 7.38-7.23 (m, 2H), 4.78- | |
| | | | | 3.47 (m, 4H), 3.30-3.02 (m, 3H), 1.54 | |
| | | | | (s, 3H), 1.44-0.56 (m, 12H). | |
| 117 | 3.49 | 478.34 | H | ¹H NMR (400 MHz, DMSO-d6) δ 8.22- | 3 |
| | | | | 8.11 (m, 2H), 7.76 (s, 1H), 7.56 (s, | |
| | | | | 1H), 7.35-7.19 (m, 2H), 5.03-3.84 | |
| | | | | (m, 4H), 3.28-3.20 (m, 0H), 1.54 (s, | |
| | | | | 3H), 1.35-0.65 (m, 12H). | |
| 118 | 3.83 | 476.39 | H | ¹H NMR (400 MHz, DMSO-d6) δ 8.21- | 3 |
| | | | | 8.11 (m, 2H), 7.76 (s, 1H), 7.56 (s, | |
| | | | | 1H), 7.36-7.24 (m, 2H), 4.83-3.40 | |
| | | | | (m, 6H), 3.29-3.17 (m, 1H), 1.54 (s, | |
| | | | | 3H), 1.35-1.07 (m, 8H), 0.96-0.87 | |
| | | | | (m, 2H), 0.86-0.72 (m, 2H), 0.61- | |
| | | | | 0.45 (m, 2H). | |
| 119 | 3.88 | 487.37 | H | ¹H NMR (400 MHz, DMSO-d6) δ 8.26- | 3 |
| | | | | 8.07 (m, 2H), 7.76 (s, 1H), 7.58 (s, | |
| | | | | 1H), 7.41-7.19 (m, 2H), 4.86-3.40 | |
| | | | | (m, 6H), 1.72-1.38 (m, 7H), 1.35- | |
| | | | | 1.10 (m, 6H), 0.96-0.82 (m, 2H). | |
| 120 | 3.54 | 506.39 | H | ¹H NMR (400 MHz, DMSO-d6) δ 8.24- | 3 |
| | | | | 8.10 (m, 2H), 7.76 (s, 1H), 7.56 (s, | |
| | | | | 1H), 7.36-7.23 (m, 2H), 4.91-3.67 | |
| | | | | (m, 7H), 3.30-3.23 (m, 1H), 2.85 (s, | |
| | | | | 1H), 1.76-1.41 (m, 7H), 1.35-1.17 | |
| | | | | (m, 2H), 1.06 (d, J = 6.7 Hz, 2H), 0.94- | |
| | | | | 0.83 (m, 2H). | |
| 121 | 3.42 | 522.38 | H | ¹H NMR (400 MHz, DMSO-d6) δ 8.20- | 3 |
| | | | | 8.08 (m, 2H), 7.75 (s, 1H), 7.65-7.46 | |
| | | | | (m, 1H), 7.36-7.24 (m, 2H), 5.68 (s, | |
| | | | | 1H), 5.32-3.40 (m, 7H), 3.28-3.12 | |
| | | | | (m, 1H), 2.11-1.73 (m, 2H), 1.67- | |
| | | | | 1.39 (m, 4H), 1.39-0.97 (m, 5H), 0.95- | |
| | | | | 0.84 (m, 2H). | |
| 122 | 3.39 | 487.53 | H | ¹H NMR (400 MHz, DMSO-d6) δ 8.20- | 3 |
| | | | | 8.10 (m, 2H), 7.78 (d, J = 2.4 Hz, 1H), | |
| | | | | 7.76 (s, 1H), 7.53 (s, 1H), 7.36-7.23 | |
| | | | | (m, 2H), 6.58 (d, J = 2.3 Hz, 1H), 5.25- | |
| | | | | 3.51 (m, 4H), 3.09 (s, 2H), 1.65-1.46 | |
| | | | | (m, 4H), 1.37-1.09 (m, 6H), 0.97- | |
| | | | | 0.85 (m, 2H). | |
| 123 | 3.69 | 516.4 | H | ¹H NMR (400 MHz, DMSO-d6) δ 8.20- | 3 |
| | | | | 8.11 (m, 2H), 7.76 (s, 1H), 7.57 (s, | |
| | | | | 1H), 7.34-7.24 (m, 2H), 6.25 (s, 1H), | |
| | | | | 4.92-3.40 (m, 7H), 3.27-2.93 (m, | |
| | | | | 2H), 2.13 (s, 3H), 1.64-1.45 (m, 4H), | |
| | | | | 1.38-1.15 (m, 5H), 0.95-0.80 (m, 2H). | |
| 124 | 3.91 | 411.14 | A | ¹H NMR (400 MHz, Methanol-d4) δ | 1 |
| | | | | 8.65-8.39 (m, 2H), 7.42 (s, 1H), 7.26- | |
| | | | | 7.04 (m, 2H), 3.89-3.76 (m, 2H), 3.59 | |
| | | | | (p, J = 6.9 Hz, 1H), 3.53-3.48 (m, 2H), | |
| | | | | 1.81 (s, 6H), 1.50 (d, J = 6.9 Hz, 6H). | |
| 125 | 4.32 | 511.52 | A | ¹H NMR (400 MHz, Methanol-d4) δ | 1 |
| | | | | 8.69-8.32 (m, 2H), 7.48 (s, 1H), 7.30- | |
| | | | | 7.07 (m, 2H), 4.67-3.84 (m, 5H), 3.70- | |
| | | | | 3.45 (m, 3H), 3.23-3.07 (m, 1H), | |
| | | | | 1.50 (dd, J = 6.9, 2.0 Hz, 8H), 1.30 | |
| | | | | (dd, J = 59.3, 6.7 Hz, 3H), 1.01 (s, 9H). | |
| 126 | 4.47 | 525.55 | A | ¹H NMR (400 MHz, Methanol-d4) δ | N/A |
| | | | | 8.59-8.34 (m, 2H), 7.38 (d, J = 3.6 | |
| | | | | Hz, 1H), 7.26-7.00 (m, 2H), 4.53 | |
| | | | | (ddd, J = 9.7, 8.6, 3.2 Hz, 1H), 4.04- | |
| | | | | 3.56 (m, 7H), 1.72-1.46 (m, 14H), | |
| | | | | 1.01 (d, J = 4.1 Hz, 9H). | |
| 127 | 3.77 | 423.35 | A | ¹H NMR (400 MHz, Methanol-d4) δ | 1 |
| | | | | 8.67-8.21 (m, 2H), 7.40 (s, 1H), 7.30- | |
| | | | | 6.94 (m, 2H), 3.94-3.71 (m, 2H), 3.61- | |
| | | | | 3.38 (m, 2H), 1.81 (s, 9H), 1.69 (q, | |
| | | | | J = 3.9 Hz, 2H), 1.11-1.04 (m, 2H). | |
| 128 | 4.63 | 523.52 | A | ¹H NMR (400 MHz, Methanol-d4) δ | 1 |
| | | | | 8.55-8.35 (m, 2H), 7.47 (s, 1H), 7.27- | |
| | | | | 7.09 (m, 2H), 4.48 (d, J = 67.5 Hz, 3H), | |
| | | | | 3.96 (s, 1H), 3.80-3.43 (m, 1H), 3.28- | |
| | | | | 3.02 (m, 3H), 1.82 (s, 3H), 1.73-1.67 | |
| | | | | (m, 2H), 1.60-1.44 (m, 2H), 1.35 (s, | |
| | | | | 1H), 1.20 (s, 2H), 1.10 (d, J = 3.2 Hz, | |
| | | | | 2H), 1.04-0.98 (m, 9H). | |
| 139 | 4.76 | 537.17 | A | ¹H NMR (400 MHz, Methanol-d4) δ | 1 |
| | | | | 8.60-8.34 (m, 2H), 7.36 (d, J = 3.7 | |
| | | | | Hz, 1H), 7.19 (t, J = 8.8 Hz, 2H), 4.62- | |
| | | | | 4.41 (m, 1H), 4.13-3.51 (m, 6H), 1.82 | |
| | | | | (d, J = 1.7 Hz, 3H), 1.76-1.68 (m, 2H), | |
| | | | | 1.65-1.46 (m, 8H), 1.09 (q, J = 3.7 | |
| | | | | Hz, 2H), 1.01 (d, J = 5.6 Hz, 9H). | |
| 130 | 3.75 | 527.44 | A | ¹H NMR (400 MHz, Methanol-d4) δ | 1 |
| | | | | 8.10 (ddt, J = 8.1, 5.2, 1.4 Hz, 2H), | |
| | | | | 7.91-7.80 (m, 1H), 7.31-7.16 (m, | |
| | | | | 2H), 4.63-4.16 (m, 3H), 4.02-3.81 | |
| | | | | (m, 1H), 3.65-3.34 (m, 1H), 3.28- | |
| | | | | 3.01 (m, 4H), 1.46-1.20 (m, 11H), | |
| | | | | 1.05-0.97 (m, 9H). | |
| 131 | 3.46 | 427.62 | A | N/A | 1 |
| 132 | 4.37 | 541.76 | A | N/A | 1 |
| 133 | 3.09 | 411.31 | A | ¹H NMR (400 MHz, Chloroform-d) δ | N/A |
| | | | | 8.19-7.97 (m, 2H), 7.64 (s, 1H), 7.17 | |
| | | | | (t, J = 8.5 Hz, 2H), 6.02 (s, 1H), 3.97 | |
| | | | | (t, J = 4.8 Hz, 2H), 3.75-3.35 (m, 3H), | |
| | | | | 1.88 (s, 6H), 1.45 (d, J = 6.9 Hz, 6H). | |
| 134 | 1.68 | 562.16 | C | ¹H NMR (400 MHz, DMSO-d6) δ 8.22- | 3 |
| | | | | 8.14 (m, 2H), 7.88 (d, J = 2.3 Hz, 1H), | |
| | | | | 7.52 (d, J = 16.0 Hz, 1H), 7.37-7.30 | |
| | | | | (m, 2H), 6.61 (d, J = 25.0 Hz, 1H), | |
| | | | | 3.95-3.87 (m, 2H), 3.69-3.60 (m, 2H), | |
| | | | | 3.56-3.49 (m, 2H), 3.47-3.37 (m, 1H), | |
| | | | | 3.09-2.96 (m, 1H), 2.71-2.56 (m, 2H), | |
| | | | | 2.46-2.36 (m, 2H), 1.50 (d, J = 10.7 | |
| | | | | Hz, 6H), 1.42 (d, J = 6.9 Hz, 6H). | |
| 135 | 1.45 | 494.69 | C | ¹H NMR (400 MHz, DMSO-d6) δ 8.22- | N/A |
| | | | | 8.14 (m, 2H), 7.88 (d, J = 2.0 Hz, 1H), | |
| | | | | 7.50 (d, J = 10.1 Hz, 1H), 7.36-7.30 | |
| | | | | (m, 2H), 5.14-5.05 (m, 1H), 4.04- | |
| | | | | 3.87 (m, 3H), 3.68-3.59 (m, 2H), 3.55 | |
| | | | | (s, 1H), 3.53-3.37 (m, 2H), 2.85-2.66 | |
| | | | | (m, 1H), 2.44-2.29 (m, 2H), 2.05- | |
| | | | | 1.89 (m, 2H), 1.51-1.46 (m, 6H), 1.44- | |
| | | | | 1.40 (m, 6H). | |
| 136 | 1.52 | 508.73 | C | ¹H NMR (400 MHz, DMSO-d6) δ 8.20- | N/A |
| | | | | 8.10 (m, 2H), 7.85 (d, J = 3.1 Hz, 1H), | |
| | | | | 7.48 (d, J = 12.6 Hz, 1H), 7.36-7.24 | |
| | | | | (m, 2H), 3.88 (t, J = 5.7 Hz, 2H), 3.66- | |
| | | | | 3.56 (m, 2H), 3.53 (s, 1H), 3.50-3.32 | |
| | | | | (m, 2H), 2.92-2.76 (m, 1H), 2.21- | |
| | | | | 1.99 (m, 4H), 1.46 (d, J = 13.1 Hz, 6H), | |
| | | | | 1.39 (dd, J = 6.9, 2.9 Hz, 6H), 1.25 (d, | |
| | | | | J = 11.8 Hz, 3H). | |
| 137 | 1.66 | 562.77 | C | ¹H NMR (400 MHz, DMSO-d6) δ 8.23- | 3 |
| | | | | 8.13 (m, 2H), 7.92-7.85 (m, 1H), 7.56- | |
| | | | | 7.47 (m, 1H), 7.40-7.27 (m, 2H), | |
| | | | | 6.62-6.50 (m, 1H), 3.99-3.86 (m, | |
| | | | | 2H), 3.68-3.38 (m, 6H), 2.61-2.52 | |
| | | | | (m, 2H), 2.29-2.16 (m, 2H), 1.54- | |
| | | | | 1.46 (m, 6H), 1.45-1.38 (m, 6H). | |
| 138 | 1.85 | 505.4 | C | ¹H NMR (400 MHz, DMSO-d6) δ 8.23- | 3 |
| | | | | 8.14 (m, 2H), 7.92-7.83 (m, 1H), 7.57- | |
| | | | | 7.50 (m, 1H), 7.39-7.27 (m, 2H), | |
| | | | | 7.00-6.83 (m, 1H), 4.08-4.01 (m, | |
| | | | | 1H), 4.01-3.95 (m, 1H), 3.94-3.89 | |
| | | | | (m, 1H), 3.80 (d, J = 9.1 Hz, 2H), 3.73- | |
| | | | | 3.66 (m, 1H), 3.49-3.37 (m, 1H), 2.31 | |
| | | | | (d, J = 6.4 Hz, 3H), 1.57 (s, 3H), 1.50 | |
| | | | | (s, 3H), 1.45-1.37 (m, 6H). | |
| 139 | 1.75 | 504.42 | C | ¹H NMR (400 MHz, DMSO-d6) δ 8.23- | 3 |
| | | | | 8.12 (m, 2H), 7.92-7.84 (m, 1H), 7.56- | |
| | | | | 7.47 (m, 2H), 7.38-7.28 (m, 2H), | |
| | | | | 6.71-6.50 (m, 1H), 4.05 (d, J = 6.5 | |
| | | | | Hz, 1H), 3.96-3.83 (m, 4H), 3.82- | |
| | | | | 3.66 (m, 4H), 3.50-3.36 (m, 1H), 1.65- | |
| | | | | 1.31 (m, 12H). | |
| 140 | 1.89 | 505.4 | C | ¹H NMR (400 MHz, DMSO-d6) δ 8.66- | 3 |
| | | | | (s, 1H), 8.23-8.14 (m, 2H), 7.91-7.85 | |
| | | | | (m, 1H), 7.55-7.51 (m, 1H), 7.37- | |
| | | | | 7.29 (m, 2H), 4.08-4.02 (m, 1H), 3.95 | |
| | | | | (t, J = 5.6 Hz, 1H), 3.86-3.77 (m, 2H), | |
| | | | | 3.76-3.64 (m, 2H), 3.48-3.37 (m, | |
| | | | | 1H), 2.17-2.09 (m, 3H), 1.60-1.35 | |
| | | | | (m, 12H). | |
| 141 | 1.74 | 504.42 | C | ¹H NMR (400 MHz, DMSO-d6) δ 8.22- | 3 |
| | | | | 8.15 (m, 2H), 7.90-7.86 (m, 1H), 7.80- | |
| | | | | 7.77 (m, 1H), 7.54-7.48 (m, 1H), | |
| | | | | 7.38-7.29 (m, 2H), 6.66-6.60 (m, | |
| | | | | 1H), 4.22 (s, 1H), 4.11 (t, J = 5.6 Hz, | |
| | | | | 1H), 4.06-3.99 (m, 1H), 3.96-3.87 | |
| | | | | (m, 4H), 3.80 (s, 1H), 3.70-3.63 (m, | |
| | | | | 1H), 3.49-3.36 (m, 1H), 1.59-1.46 | |
| | | | | (m, 6H), 1.46-1.34 (m, 6H). | |
| 142 | 1.54 | 491.44 | C | ¹H NMR (400 MHz, DMSO-d6) δ 8.49- | 3 |
| | | | | (s, 1H), 8.24-8.14 (m, 2H), 7.93-7.83 | |
| | | | | (m, 1H), 7.57-7.48 (m, 1H), 7.41- | |
| | | | | 7.26 (m, 2H), 4.35-3.90 (m, 5H), 3.84 | |
| | | | | (s, 1H), 3.78-3.67 (m, 1H), 3.51-3.32 | |
| | | | | (m, 1H), 1.53 (d, J = 31.6 Hz, 6H), | |
| | | | | 1.46-1.37 (m, 6H). | |
| 143 | 5.87 | 560.6 | D | ¹H NMR (400 MHz, Chloroform-d) δ | 3 |
| | | | | 8.04-7.92 (m, 2H), 7.62 (d, J = 0.8 | |
| | | | | Hz, 1H), 7.44 (d, J = 2.7 Hz, 1H), 7.21- | |
| | | | | 7.12 (m, 2H), 6.02 (dt, J = 5.8, 1.1 | |
| | | | | Hz, 1H), 5.62 (dt, J = 3.4, 1.5 Hz, 1H), | |
| | | | | 4.12 (s, 1H), 3.95 (td, J = 7.2, 5.5 Hz, | |
| | | | | 2H), 3.79-3.70 (m, 2H), 3.66 (t, J = | |
| | | | | 5.7 Hz, 1H), 3.46 (s, 1H), 3.18-3.01 | |
| | | | | (m, 1H), 2.84-2.71 (m, 2H), 2.61- | |
| | | | | 2.45 (m, 2H), 2.37-2.29 (m, 3H), 1.61 | |
| | | | | (d, J = 4.5 Hz, 6H). | |
| 144 | 1.78 | 540.74 | C | ¹H NMR (400 MHz, DMSO-d6) δ 8.21- | 12 |
| | | | | 8.14 (m, 2H), 7.90-7.86 (m, 3H), 7.51 | |
| | | | | (s, 1H), 7.37-7.29 (m, 2H), 3.77-3.70 | |
| | | | | (m, 5H), 3.43-3.35 (m, 3H), 3.12 (s, | |
| | | | | 2H), 1.53-1.48 (m, 6H), 1.43-1.36 | |
| | | | | (m, 6H). | |
| 145 | 1.71 | 554.37 | C | ¹H NMR (400 MHz, DMSO-d6) δ 8.23- | 12 |
| | | | | 8.13 (m, 2H), 7.88 (s, 1H), 7.53 (s, | |
| | | | | 1H), 7.51-7.47 (m, 1H), 7.33 (t, J = | |
| | | | | 8.9 Hz, 2H), 3.82 (t, J = 5.3 Hz, 2H), | |
| | | | | 3.70 (s, 3H), 3.42-3.32 (m, 3H), 3.25 | |
| | | | | (s, 2H), 2.39 (s, 3H), 1.55-1.46 (m, | |
| | | | | 6H), 1.44-1.35 (m,6H). | |
| 146 | 1.94 | 554.7 | C | ¹H NMR (400 MHz, DMSO-d6) δ 8.27- | 12 |
| | | | | (s, 1H), 8.21-8.14 (m, 2H), 7.87 (s, | |
| | | | | 1H), 7.54 (s, 1H), 7.36-7.28 (m, 2H), | |
| | | | | 3.84 (s, 3H), 3.75 (t, J = 5.3 Hz, 2H), | |
| | | | | 3.44-3.36 (m, 1H), 3.25-3.19 (m, | |
| | | | | 2H), 3.04-2.97 (m, 2H), 2.32 (s, 3H), | |
| | | | | 1.54-1.48 (m, 6H), 1.43-1.36 (m, 6H). | |
| 147 | 1.78 | 526.7 | C | ¹H NMR (400 MHz, DMSO-d6) δ 8.41- | 12 |
| | | | | (s, 1H), 8.21-8.12 (m, 2H), 7.87 (s, | |
| | | | | 1H), 7.52 (s, 1H), 7.37-7.27 (m, 2H), | |
| | | | | 3.78-3.69 (m, 2H), 3.50-3.30 (m, | |
| | | | | 3H), 3.26-3.17 (m, 2H), 2.99 (s, 2H), | |
| | | | | 1.55-1.47 (m, 6H), 1.44-1.36 (m, 6H). | |
| 148 | 1.92 | 540.7 | C | ¹H NMR (400 MHz, DMSO-d6) δ 8.21- | 12 |
| | | | | 8.13 (m, 2H), 8.02-7.98 (m, 1H), 7.87 | |
| | | | | (s, 1H), 7.52 (s, 1H), 7.37-7.29 (m, | |
| | | | | 2H), 6.73 (d, J = 2.3 Hz, 1H), 3.98 (s, | |
| | | | | 3H), 3.80-3.73 (m, 2H), 3.45-3.34 | |
| | | | | (m, 3H), 3.12 (s, 2H), 1.54-1.47 (m, | |
| | | | | 6H), 1.42-1.37 (m, 6H). | |
| 149 | 1.67 | 423.94 | C | ¹H NMR (400 MHz, Methanol-d4) δ | 1 |
| | | | | 8.05-7.96 (m, 2H), 7.71 (s, 1H), 7.45 | |
| | | | | (s, 1H), 7.28-7.17 (m, 2H), 3.90-3.84 | |
| | | | | (m, 2H), 3.56-3.49 (m, 2H), 2.79 (s, | |
| | | | | 1H), 1.82 (s, 6H), 1.57 (s, 9H). | |
| 150 | 3.45 | 548.4 | A | ¹H NMR (400 MHz, DMSO-d6) δ 8.22- | 3 |
| | | | | 8.13 (m, 2H), 7.93-7.87 (m, 1H), 7.51 | |
| | | | | (d, J = 16.1 Hz, 1H), 7.33 (ddd, J = 9.0, | |
| | | | | 7.9, 0.8 Hz, 2H), 6.59 (d, J = 21.8 Hz, | |
| | | | | 1H), 3.90 (dd, J = 6.8, 3.9 Hz, 2H), | |
| | | | | 3.70-3.59 (m, 2H), 3.52 (d, J = 6.3 | |
| | | | | Hz, 2H), 3.09-2.92 (m, 3H), 2.69- | |
| | | | | 2.56 (m, 2H), 2.41 (t, J = 11.1 Hz, 2H), | |
| | | | | 1.50 (d, J = 10.4 Hz, 6H), 1.42-1.33 | |
| | | | | (m, 3H). | |
| 151 | 1.7 | 522.4 | C | ¹H NMR (400 MHz, DMSO-d6) δ 8.19- | 3 |
| | | | | 8.08 (m, 2H), 7.72 (d, J = 2.6 Hz, 1H), | |
| | | | | 7.48 (d, J = 11.7 Hz, 1H), 7.34-7.23 | |
| | | | | (m, 2H), 4.96 (d, J = 18.8 Hz, 1H), 3.89 | |
| | | | | (s, 2H), 3.64-3.51 (m, 4H), 3.49-3.43 | |
| | | | | (m, 1H), 2.20-2.02 (m, 4H), 1.50 (d, | |
| | | | | J = 3.1 Hz, 9H), 1.46 (d, J = 12.8 Hz, | |
| | | | | 6H), 1.25 (d, J = 12.0 Hz, 3H). | |
| 152 | 1.67 | 504.39 | C | ¹H NMR (400 MHz, DMSO-d6) δ 8.21- | 3 |
| | | | | 8.06 (m, 2H), 7.84-7.78 (m, 1H), 7.75- | |
| | | | | 7.67 (m, 1H), 7.52-7.45 (m, 1H), | |
| | | | | 7.33-7.24 (m, 2H), 6.68-6.54 (m, 1H), | |
| | | | | 4.25 (s, 1H), 4.16-3.64 (m, 6H), 1.58- | |
| | | | | 1.49 (m, 9H), 1.46 (d, J = 4.7 Hz, 6H). | |
| 153 | 1.7 | 518.39 | C | ¹H NMR (400 MHz, DMSO-d6) δ 8.22- | 3 |
| | | | | 8.06 (m, 2H), 7.76-7.68 (m, 1H), 7.52- | |
| | | | | 7.44 (m, 1H), 7.34-7.23 (m, 2H), | |
| | | | | 6.39-6.31 (m, 1H), 4.24 (s, 1H), 4.14- | |
| | | | | 3.59 (m, 6H), 2.66 (s, 3H), 1.51 (s, | |
| | | | | 9H), 1.46 (d, J = 10.3 Hz, 6H). | |
| 154 | 1.7 | 505.5 | C | ¹H NMR (400 MHz, DMSO-d6) δ 8.68 | 3 |
| | | | | (s, 1H), 8.14 (dd, J = 8.8, 5.7 Hz, 2H), | |
| | | | | 7.74-7.67 (m, 1H), 7.54-7.44 (m, | |
| | | | | 1H), 7.37-7.23 (m, 2H), 4.48 (s, 1H), | |
| | | | | 4.32-3.62 (m, 6H), 1.60-1.38 (m, 15H). | |
| 155 | 0.99 | 460.12 | B | ¹H NMR (400 MHz, Chloroform-d) δ | N/A |
| | | | | 8.10 (d, J = 8.5 Hz, 2H), 7.73 (d, J = | |
| | | | | 8.4 Hz, 2H), 7.61 (s, 1H), 7.49-7.42 | |
| | | | | (m, 1H), 6.03 (s, 1H), 3.86-3.80 (m, | |
| | | | | 2H), 3.63-3.56 (m, 2H), 3.44 (p, J = | |
| | | | | 6.9 Hz, 1H), 1.87 (s, 6H), 1.46 (d, J = | |
| | | | | 6.9 Hz, 6H). | |
| 156 | 1.07 | 474.45 | I | ¹H NMR (400 MHz, DMSO-d6) δ 8.37- | 1 |
| | | | | 8.28 (m, 2H), 8.17 (t, J = 3.3 Hz, 1H), | |
| | | | | 7.90-7.78 (m, 3H), 7.59 (s, 1H), 3.81- | |
| | | | | 3.69 (m, 2H), 3.42-3.33 (m, 2H), 1.67 | |
| | | | | (s, 6H), 1.51 (s, 9H). | |
| 157 | 1.99 | 440.41 | C | ¹H NMR (400 MHz, DMSO-d6) δ 8.22- | 1 |
| | | | | 8.18 (m, 1H), 8.17-8.10 (m, 2H), 7.77 | |
| | | | | (s, 1H), 7.58 (s, 1H), 7.57-7.53 (m, | |
| | | | | 2H), 3.80-3.68 (m, 2H), 3.43-3.34 | |
| | | | | (m, 2H), 1.69 (s, 6H), 1.51 (s, 9H). | |
| 158 | 1.09 | 572.31 | I | ¹H NMR (400 MHz, DMSO-d6) δ 8.32- | 3 |
| | | | | (d, 2H), 7.87-7.80 (m, 3H), 7.53 (d, | |
| | | | | J = 11.8 Hz, 1H), 4.96 (d, J = 19.0 Hz, | |
| | | | | 1H), 3.95-3.85 (m, 2H), 3.65-3.57 | |
| | | | | (m, 2H), 3.54 (s, 1H), 3.47 (t, J = 5.8 | |
| | | | | Hz, 1H), 2.83 (tt, J = 17.4, 8.8 Hz, 1H), | |
| | | | | 2.22-2.02 (m, 4H), 1.51 (d, J = 3.0 | |
| | | | | Hz, 9H), 1.46 (d, J = 12.9 Hz, 6H), | |
| | | | | 1.25 (d, J = 12.0 Hz, 3H). | |
| 159 | 2.03 | 538.51 | C | ¹H NMR (400 MHz, DMSO-d6) δ 8.19- | 3 |
| | | | | 8.11 (m, 2H), 7.77 (s, 0.5H), 7.76 (s, | |
| | | | | 0.5H), 7.59-7.53 (m, 2H), 7.53 (s, | |
| | | | | 0.5H), 7.50 (s, 0.5H), 5.00 (s, 0.5H), | |
| | | | | 4.95 (s, 0.5H), 3.97-3.86 (m, 2H), | |
| | | | | 3.70-3.59 (m, 2H), 3.55 (s, 1H), 3.52- | |
| | | | | 3.44 (m, 1H), 2.95-2.76 (m, 1H), 2.22- | |
| | | | | 2.14 (m, 2H), 2.13-2.05 (m, 2H), | |
| | | | | 1.52 (s, 4.5H), 1.52 (s, 4.5H), 1.50 (s, | |
| | | | | 3H), 1.47 (s, 3H), 1.29 (s, 1.5H), 1.26 | |
| | | | | (s, 1.5H). Signals doubled due to | |
| | | | | presence of rotamers, | |
| 160 | 1.90 | 519.16 | C | ¹H NMR (400 MHz, DMSO-d6) δ 8.26- | 3 |
| | | | | 8.10 (m, 2H), 7.96 (s, 1H), 7.77 (s, | |
| | | | | 1H), 7.39-7.26 (m, 2H), 4.57-3.60 | |
| | | | | (m, 6H), 2.74 (s, 3H), 1.62-1.45 (m, | |
| | | | | 15H). | |
| 161 | 3.47 | 438.13 | A | ¹H NMR (400 MHz, DMSO-d6) δ 8.20 | 1 |
| | | | | (t, J = 3.2 Hz, 1H), 8.06 (ddd, J = 7.7, | |
| | | | | 2.4, 0.9 Hz, 1H), 8.00-7.91 (m, 1H), | |
| | | | | 7.75 (s, 1H), 7.57 (s, 1H), 7.25 (dd, | |
| | | | | J = 9.6, 8.6 Hz, 1H), 3.77 (dd, J = | |
| | | | | 6.5, 3.4 Hz, 2H), 3.39 (dt, J = 6.4, | |
| | | | | 3.6 Hz, 2H), 2.34 (d, J = 1.9 Hz, | |
| | | | | 3H), 1.70 (s, 6H), 1.52 (s, 9H). | |
| 162 | 3.3 | 519.09 | A | ¹H NMR (400 MHz, DMSO-d6) δ 8.68 | 3 |
| | | | | (s, 1H), 8.07-7.99 (m, 1H), 7.94 (ddd, | |
| | | | | J = 8.0, 5.2, 2.4 Hz, 1H), 7.71 (d, J = | |
| | | | | 8.5 Hz, 1H), 7.48 (d, J = 9.8 Hz, 1H), | |
| | | | | 7.22 (td, J = 9.1, 3.7 Hz, 1H), 4.48 (s, | |
| | | | | 1H), 4.10-3.61 (m, 6H), 2.31 (t, J = | |
| | | | | 2.5 Hz, 3H), 1.61-1.39 (m, 15H). | |
| | | | | Signals doubled due to amide rotamers, | |
| 163 | 3.55 | 537.76 | A | N/A | 3 |
| 164 | 3.44 | 518.16 | A | N/A | 3 |
| 165 | 3.36 | 420.1 | A | ¹H NMR (400 MHz, DMSO-d6) δ 8.24- | 1 |
| | | | | 8.15 (m, 1H), 8.04-7.96 (m, 2H), 7.73 | |
| | | | | (s, 1H), 7.57 (s, 1H), 7.35-7.27 (m, | |
| | | | | 2H), 3.82-3.73 (m, 2H), 3.40 (dd, J = | |
| | | | | 6.1, 3.2 Hz, 2H), 2.37 (s, 3H), 1.70 (s, | |
| | | | | 6H), 1.52 (s, 9H). | |
| 166 | 3.32 | 533.2 | A | N/A | 3 |
| 167 | 3.5 | 518.1 | A | N/A | 3 |
| 168 | 1.06 | 520.53 | I | ¹H NMR (400 MHz, DMSO-d6) δ | 3 |
| | | | | 13.25 (broad s, 1H), 8.25-8.07 (m, 2H), | |
| | | | | 7.93-7.69 (m, 2H), 7.62-7.43 (m, 3H), | |
| | | | | 6.73-6.54 (m, 1H), 4.27 (broad s, 1H), | |
| | | | | 4.17-4.00 (m, 2H), 3.95 (broad s, 1H), | |
| | | | | 3.81 (s, 1H), 3.75-3.62 (m, 1H), 1.78- | |
| | | | | 1.16 (m, 15H). | |
| 169 | 0.93 | 521.56 | I | ¹H NMR (400 MHz, DMSO-d6) δ 8.53 | |
| | | | | (broad s, 1H), 8.22-8.08 (m, 2H), 7.77 | |
| | | | | (2s, 1H), 7.64-7.46 (m, 3H), 4.15- | |
| | | | | 4.00 (m, 2H), 3.95 (broad s, 1H), 3.83 | |
| | | | | (s, 1H), 3.79-3.65 (m, 1H), 1.55 (2s, | |
| | | | | 9H), 1.48 (s, 6H). Signals doubled due | |
| | | | | to amide rotamers, | |
| 170 | 0.83 | 568.53 | I | ¹H NMR (400 MHz, DMSO-d6) δ 8.32 | 3 |
| | | | | d, J = 8.1 Hz, 2H), 7.90-7.77 (m, 3H), | |
| | | | | 7.58-7.42 (m, 2H), 4.55-3.91 (m, 4H), | |
| | | | | 3.62 (d, 2H), 2.29 (s, 3H), 1.50 (s, | |
| | | | | 15H). | |
| 171 | 1.11 | 568.53 | I | ¹H NMR (400 MHz, DMSO-d6) δ 8.42- | 3 |
| | | | | 8.24 (m, 2H), 7.96-7.71 (m, 3H), 7.53 | |
| | | | | (d, J = 7.4 Hz, 1H), 6.36 (d, J = 8.2 Hz, | |
| | | | | 1H), 4.25 (s, 1H), 4.09 (s, 1H), 4.00 (t, | |
| | | | | J = 5.8 Hz, 1H), 3.91 (s, 1H), 3.77 (s, | |
| | | | | 1H), 3.64 (t, J = 5.8 Hz, 1H), 2.23 (d, | |
| | | | | J = 4.0 Hz, 3H), 1.52 (s, 8H), 1.49 (s, | |
| | | | | 4H), 1.45 (s, 4H). | |
| 172 | 1.06 | 554.52 | I | ¹H NMR (400 MHz, DMSO-d6) δ 8.32 | 3 |
| | | | | (dd, J = 8.2, 3.9 Hz, 2H), 7.89-7.78 | |
| | | | | (m, 4H), 7.54 (d, J = 9.2 Hz, 1H), 6.68- | |
| | | | | 6.57 (m, 1H), 4.25 (s, 1H), 4.11 (s, | |
| | | | | 1H), 4.01 (t, J = 5.8 Hz, 1H), 3.92 (s, | |
| | | | | 1H), 3.79 (s, 1H), 3.67 (t, J = 5.7 Hz, | |
| | | | | 1H), 1.53 (s, 8H), 1.47 (d, J = 8.7 Hz, | |
| | | | | 8H). | |
| 173 | 0.94 | 555.5 | I | ¹H NMR (400 MHz, DMSO-d6) δ 8.46 | 3 |
| | | | | (s, 1H), 8.36-8.25 (m, 2H), 7.84 (dd, | |
| | | | | J = 8.3, 4.6 Hz, 3H), 7.54 (d, J = 9.7 | |
| | | | | Hz, 1H), 4.22-3.97 (m, 3H), 3.92 (d, | |
| | | | | J = 5.8 Hz, 1H), 3.82 (s, 1H), 3.71 (t, | |
| | | | | J = 5.7 Hz, 1H), 1.54 (d, J = 7.4 Hz, | |
| | | | | 8H), 1.47 (d, J = 3.9 Hz, 7H). | |
| 174 | 0.95 | 569.51 | I | ¹H NMR (400 MHz, DMSO-d6) δ 8.42- | 3 |
| | | | | 8.23 (m, 2H), 7.92-7.75 (m, 3H), 7.54 | |
| | | | | (d, J = 8.7 Hz, 1H), 4.02 (t, J = 5.8 Hz, | |
| | | | | 2H), 3.92 (s, 1H), 3.79 (s, 1H), 3.68 (t, | |
| | | | | J = 5.8 Hz, 1H), 2.35 (d, J = 8.7 Hz, | |
| | | | | 3H), 1.53 (d, J = 4.0 Hz, 8H), 1.47 (d, | |
| | | | | J = 9.3 Hz, 7H). | |
| 175 | 1.26 | 570.49 | I | ¹H NMR (400 MHz, DMSO-d6) δ 8.40- | 3 |
| | | | | 8.25 (m, 2H), 7.91-7.78 (m, 3H), 7.56 | |
| | | | | (d, J = 4.5 Hz, 1H), 4.22-4.11 (m, 2H), | |
| | | | | 4.11-3.97 (m, 2H), 3.83 (s, 1H), 3.76- | |
| | | | | 3.67 (m, 1H), 2.57 (d, J = 3.4 Hz, 3H), | |
| | | | | 1.55 (s, 3H), 1.54-1.49 (m, 12H). | |
| 176 | 1.11 | 458.44 | I | ¹H NMR (400 MHz, DMSO-d6) δ 8.23- | 1 |
| | | | | 8.13 (m, 2H), 8.03 (dd, J = 8.4, 1.9 Hz, | |
| | | | | 1H), 7.83 (s, 1H), 7.71 (t, J = 8.1 Hz, | |
| | | | | 1H), 7.59 (s, 1H), 3.82-3.71 (m, 2H), | |
| | | | | 3.45-3.34 (m, 2H), 1.69 (s, 6H), 1.52 | |
| | | | | (s, 9H). | |
| 177 | 1.08 | 534.54 | I | ¹H NMR (400 MHz, DMSO-d6) δ | 3 |
| | | | | 12.90 (broad s, 1H), 8.23-8.07 (m, 2H), | |
| | | | | 7.76 (2s, 1H), 7.61-7.45 (m, 3H), 6.37 | |
| | | | | (2s, 1H), 4.26 (s, 1H), 4.15-3.53 (m, | |
| | | | | 5H), 2.25 (2s, 3H), 1.53 (s, 9H), 1.48 | |
| | | | | (2s, 6H). | |
| 178 | 0.69 | 535.52 | I | ¹H NMR (400 MHz, DMSO-d6) δ 8.25- | 3 |
| | | | | 8.07 (m, 2H), 7.76 (2s, 1H), 7.62-7.45 | |
| | | | | (m, 3H), 4.10-3.85 (m, 4H), 3.81 (s, | |
| | | | | 1H), 3.75-3.65 (m, 1H), 2.38 (2s, 3H), | |
| | | | | 1.54 (2s, 9H), 1.48 (2s, 6H). Signals | |
| | | | | doubled due to amide rotamers, | |
| 179 | 0.98 | 536.5 | I | ¹H NMR (400 MHz, DMSO-d6) δ 8.19- | 3 |
| | | | | 8.09 (m, 2H), 7.77 (2s, 1H), 7.59-7.52 | |
| | | | | (m, 3H), 4.22-3.98 (m, 4H), 3.85 (s, | |
| | | | | 1H), 3.73-3.69 (m, 1H), 2.59 (2s, 3H), | |
| | | | | 1.54 (3s, 15H). | |
| 180 | 0.51 | 534.59 | I | ¹H NMR (400 MHz, DMSO-d6) δ | 3 |
| | | | | 12.29 (2s, 1H), 8.23-8.06 (m, 2H), 7.77 | |
| | | | | (s, 1H), 7.63-7.44 (m, 4H), 4.64-3.52 | |
| | | | | (m, 6H), 2.28 (2s, 3H), 1.51 (2s, 15H). | |
| 181 | 0.77 | 539.49 | I | ¹H NMR (400 MHz, DMSO-d6) δ 8.22- | 1 |
| | | | | 8.10 (m, 1H), 8.07-7.96 (m, 1H), 7.82 | |
| | | | | (2s, 1H), 7.75-7.65 (m, 1H), 7.53 (2s, | |
| | | | | 1H), 4.13-3.89 (m, 4H), 3.83 (s, 1H), | |
| | | | | 3.79-3.68 (m, 1H), 1.55 (2s, 9H), 1.49 | |
| | | | | (s, 6H). Signals doubled due to amide | |
| | | | | rotamers, | |
| 182 | 4.95 | 490.26 | D | ¹H NMR (400 MHz, DMSO-d6) δ | 1 |
| | | | | 12.79 (s, 1H), 8.20-8.05 (m, 2H), 8.02 | |
| | | | | (s, 1H), 7.74 (s, 2H), 7.60 (s, 1H), 7.31 | |
| | | | | (t, J = 8.8 Hz, 2H), 4.05-3.79 (m, 4H), | |
| | | | | 1.77 (s, 6H), 1.52 (s, 9H). | |
| 183 | 5.27 | 519.97 | D | ¹H NMR (400 MHz, DMSO-d6) δ 8.14 | 3 |
| | | | | (tdd, J = 6.9, 3.2, 1.4 Hz, 2H), 7.72 | |
| | | | | (dd, J = 4.4, 1.4 Hz, 1H), 7.51 (dd, J = | |
| | | | | 4.4, 1.4 Hz, 1H), 7.35-7.20 (m, 2H), | |
| | | | | 4.20-4.11 (m, 2H), 4.08-3.96 (m, | |
| | | | | 2H), 3.83 (s, 1H), 3.76-3.65 (m, 1H), | |
| | | | | 2.57 (s, 3H), 1.60-1.41 (m, 15H). | |
| 184 | 4.92 | 515.97 | D | ¹H NMR (400 MHz, DMSO-d6) δ 8.97- | 3 |
| | | | | 8.82 (m, 2H), 8.21-8.02 (m, 2H), 7.76- | |
| | | | | 7.66 (m, 1H), 7.65-7.58 (m, 1H), | |
| | | | | 7.54-7.44 (m, 1H), 7.40-7.19 (m, | |
| | | | | 2H), 4.09-3.96 (m, 1H), 3.88-3.77 | |
| | | | | (m, 2H), 3.77-3.67 (m, 1H), 3.49- | |
| | | | | 3.41 (m, 1H), 3.39 (s, 1H), 1.60-1.46 | |
| | | | | (m, 9H), 1.46-1.27 (m, 6H). | |
| 185 | 5.23 | 515.62 | D | ¹H NMR (400 MHz, DMSO-d6) δ 8.67- | 3 |
| | | | | 8.51 (m, 1H), 8.20-8.04 (m, 2H), 8.03- | |
| | | | | 7.87 (m, 1H), 7.71 (d, J = 18.2 Hz, | |
| | | | | 1H), 7.62 (d, J = 3.4 Hz, 1H), 7.55- | |
| | | | | 7.39 (m, 2H), 7.29 (q, J = 8.6 Hz, 2H), | |
| | | | | 4.11-3.97 (m, 1H), 3.91-3.83 (m, | |
| | | | | 1H), 3.80 (s, 1H), 3.75-3.65 (m, 3H), | |
| | | | | 1.60-1.45 (m, 6H), 1.40 (s, 6H). | |
| 186 | 5.68 | 519.68 | D | ¹H NMR (400 MHz, DMSO-d6) δ 8.13 | 3 |
| | | | | (dt, J = 8.2, 5.5 Hz, 2H), 7.72 (d, J = | |
| | | | | 7.2 Hz, 1H), 7.49 (d, J = 7.7 Hz, 1H), | |
| | | | | 7.30 (td, J = 8.8, 3.3 Hz, 2H), 6.56- | |
| | | | | 6.40 (m, 1H), 4.10-3.96 (m, 1H), 3.96- | |
| | | | | 3.85 (m, 1H), 3.85-3.77 (m, 3H), | |
| | | | | 3.76-3.60 (m, 1H), 2.53-2.34 (m, | |
| | | | | 3H), 1.58-1.34 (m, 15H). | |
| 187 | 4.58 | 521.53 | D | ¹H NMR (400 MHz, DMSO-d6) δ | 3 |
| | | | | 12.14-11.86 (m, 2H), 8.23-8.01 (m, | |
| | | | | 2H), 7.72 (d, J = 3.8 Hz, 1H), 7.49 (d, | |
| | | | | J = 5.0 Hz, 1H), 7.30 (t, J = 8.7 Hz, | |
| | | | | 2H), 4.22 (s, 1H), 4.10 (s, 1H), 4.01 (s, | |
| | | | | 1H), 3.95 (d, J = 5.6 Hz, 1H), 3.77 (s, | |
| | | | | 1H), 3.72-3.57 (m, 1H), 1.65-1.35 (m, | |
| | | | | 15H). | |
| 188 | 5.45 | 550.62 | D | ¹H NMR (400 MHz, DMSO-d6) δ 8.25- | 3 |
| | | | | 8.04 (m, 2H), 7.72 (t, J = 1.5 Hz, 1H), | |
| | | | | 7.51-7.42 (m, 1H), 7.35-7.22 (m, | |
| | | | | 2H), 3.90 (d, J = 5.8 Hz, 2H), 3.66- | |
| | | | | 3.40 (m, 8H), 3.06 (td, J = 9.4, 4.3 Hz, | |
| | | | | 1H), 2.43-2.22 (m, 4H), 1.55-1.36 | |
| | | | | (m, 15H). | |
| 189 | 1.66 | 536.5 | C | ¹H NMR (400 MHz, DMSO-d6) δ 8.19- | 13 |
| | | | | 8.08 (m, 2H), 7.71 (d, J = 1.5 Hz, 1H), | |
| | | | | 7.48 (d, J = 9.4 Hz, 1H), 7.30 (M, 2H), | |
| | | | | 3.94-3.85 (m, 2H), 3.63-3.45 (m, | |
| | | | | 5H), 3.05-2.88 (m, 1H), 2.44-2.24 (m, | |
| | | | | 4H), 1.52-1.44 (m, 15H). | |
| 190 | 1.87 | 562.54 | C | ¹H NMR (400 MHz, DMSO-d6) δ 8.04- | 3 |
| | | | | 7.94 (m, 2H), 7.71-7.65 (m, 1H), 7.43- | |
| | | | | 7.35 (m, 1H), 7.26-7.13 (m, 2H), 4.10- | |
| | | | | 3.98 (m, 2H), 3.90 (t, J = 5.6 Hz, 1H), | |
| | | | | 3.80 (s, 1H), 3.74 (s, 1H), 3.70-3.59 (m, | |
| | | | | 4H), 2.41 (d, J = 9.1 Hz, 6H), 1.64-1.52 | |
| | | | | (m, 15H). | |
| 191 | 1.66 | 548.21 | C | ¹H NMR (400 MHz, DMSO-d6) δ | 13 |
| | | | | 12.47 (s, 1H), 8.21-8.06 (m, 2H), 7.72 | |
| | | | | (d, J = 4.0 Hz, 1H), 7.49 (d, J = 15.7 | |
| | | | | Hz, 1H), 7.36-7.24 (m, 2H), 4.00-3.83 | |
| | | | | (m, 2H), 3.82-3.75 (m, 1H), 3.69 (s, | |
| | | | | 1H), 3.62 (s, 1H), 3.49-3 41 (m, 1H), | |
| | | | | 2.32-2.20 (m, 6H), 1.56-1.43 (m, 15H). | |
| 192 | 1.68 | 558.49 | C | ¹H NMR (400 MHz, DMSO-d6) δ 8.24- | 13 |
| | | | | 8.08 (m, 2H), 8.06-7.91 (m, 2H), | |
| | | | | 7.72 (2s, 1H), 7.63-7.42 (m, 3H), 7.39- | |
| | | | | 7.18 (m, 2H), 4.15-3.65 (m, 4H), 3.52 | |
| | | | | (s, 2H), 1.57 (2s, 9H), 1.40 (2s, 6H). | |
| | | | | rotamers, | |
| 193 | 1.59 | 522.5 | C | ¹H NMR (400 MHz, DMSO-d6) δ 8.22- | 13 |
| | | | | 8.09 (m, 2H), 7.74 (s, 1H), 7.50 (2s, | |
| | | | | 1H), 7.32 (t, J = 8.8 Hz, 2H), 4.02-3.87 | |
| | | | | (m, 2H), 3.80-3.71 (m, 1H), 3.68-3.59 | |
| | | | | (m, 2H), 3.56-3.45 (m, 1H), 1.57-1.46 | |
| | | | | (m, 15H), 1.39-1.33 (m, 1H), 1.33-1.28 | |
| | | | | (m, 1H), 1.27-1.15 (m, 2H). rotamers, | |
| 194 | 3.19 | 510.18 | A | ¹H NMR (400 MHz, DMSO-d6) δ | 13 |
| | | | | 12.06 (s, 1H), 8.20-8.13 (m, 2H), 7.75 | |
| | | | | (d, J = 1.7 Hz, 1H), 7.52 (d, J = 10.6 | |
| | | | | Hz, 1H), 7.33 (td, J = 8.9, 1.2 Hz, 2H), | |
| | | | | 3.99 (t, J = 5.6 Hz, 1H), 3.92 (dd, J = | |
| | | | | 7.2, 4.4 Hz, 1H), 3.77-3.70 (m, 1H), | |
| | | | | 3.66 (s, 2H), 3.52 (dd, J = 6.9, 4.4 Hz, | |
| | | | | 1H), 2.59 (dd, J = 7.5, 5.5 Hz, 1H), | |
| | | | | 2.53 (dd, J = 6.0, 1.3 Hz, 1H), 2.47 | |
| | | | | (dd, J = 7.5, 5.5 Hz, 2H), 1.57-1.46 | |
| | | | | (m, 15H). | |
| 195 | 3.45 | 524.18 | A | ¹H NMR (400 MHz, DMSO-d6) δ | 13 |
| | | | | 13.03 (s, 1H), 8.17 (dd, J = 8.9, 5.5 | |
| | | | | Hz, 2H), 7.75 (s, 1H), 7.51 (s, 1H), | |
| | | | | 7.33 (t, J = 8.9 Hz, 2H), 3.93 (s, 2H), | |
| | | | | 3.56 (s, 4H), 1.53 (s, 15H), 1.33 (s, 6H). | |
| 196 | 3.55 | 538.22 | A | ¹H NMR (400 MHz, DMSO-d6) δ | 13 |
| | | | | 11.62 (s, 1H), 8.25-8.09 (m, 2H), 7.75 | |
| | | | | (d, J = 2.7 Hz, 1H), 7.52 (d, J = 15.1 | |
| | | | | Hz, 1H), 7.33 (td, J = 8.9, 2.0 Hz, 2H), | |
| | | | | 3.94 (dt, J = 22.1, 5.8 Hz, 2H), 3.70 (t, | |
| | | | | J = 5.5 Hz, 1H), 3.64 (d, J = 3.6 Hz, | |
| | | | | 2H), 3.49 (dd, J = 7.0, 4.5 Hz, 1H), | |
| | | | | 2.60 (d, J = 23.1 Hz, 2H), 1.56-1.51 | |
| | | | | (m, 12H), 1.48 (s, 3H), 1.18 (d, J = | |
| | | | | 2.1 Hz, 6H). | |
| 197 | 3.27 | 522.17 | A | ¹H NMR (400 MHz, DMSO-d6) δ | 13 |
| | | | | 12.53 (s, 1H), 8.27-8.06 (m, 2H), 7.75 | |
| | | | | (d, J = 1.3 Hz, 1H), 7.52 (d, J = 12.2 | |
| | | | | Hz, 1H), 7.39-7.24 (m, 2H), 4.07-3.81 | |
| | | | | (m, 4H), 3.67 (s, 1H), 3.55 (q, J = 5.4 | |
| | | | | Hz, 1H), 2.35 (dddd, J = 75.1, 9.3, 6.0, | |
| | | | | 3.8 Hz, 1H), 1.95-1.84 (m, 1H), 1.61- | |
| | | | | 1.44 (m, 15H), 1.25 (tdd, J = 8.9, 5.8, | |
| | | | | 3.3 Hz, 2H). | |
| 198 | 3.76 | 578.31 | A | ¹H NMR (400 MHz, DMSO-d6) δ | 13 |
| | | | | 12.18 (s, 1H), 8.17 (ddd, J = 9.1, 5.5, | |
| | | | | 2.4 Hz, 2H), 7.75 (d, J = 3.8 Hz, 1H), | |
| | | | | 7.52 (d, J = 14.9 Hz, 1H), 7.33 (td, J = | |
| | | | | 8.9, 2.2 Hz, 2H), 3.93 (dt, J = 16.1, 6.0 | |
| | | | | Hz, 2H), 3.77 (t, J = 5.5 Hz, 1H), 3.70 | |
| | | | | (s, 1H), 3.63 (s, 1H), 3.48 (dd, J = 7.0, | |
| | | | | 4.4 Hz, 1H), 2.08 (dd, J = 8.6, 5.8 Hz, | |
| | | | | 2H), 1.66-1.34 (m, 20H), 1.29-1.05 | |
| | | | | (m, 5H). | |
| 199 | 3.1 | 521.1 | A | ¹H NMR (400 MHz, DMSO-d6) δ 8.17 | 3 |
| | | | | (dd, J = 8.6, 5.6 Hz, 2H), 7.75 (s, 1H), | |
| | | | | 7.51 (d, J = 6.7 Hz, 1H), 7.33 (t, J = 8.8 | |
| | | | | Hz, 2H), 7.27-7.03 (m, 2H), 4.04- | |
| | | | | 3.88 (m, 2H), 3.78-3.49 (m, 4H), 1.53 | |
| | | | | (s, 15H), 1.27 (q, J = 4.0, 3.6 Hz, 2H), | |
| | | | | 1.11 (d, J = 11.3 Hz, 2H). | |
| 200 | 5.53 | 513.85 | D | ¹H NMR (400 MHz, DMSO-d6) δ 8.14 | 12 |
| | | | | (dd, J = 8.7, 5.6 Hz, 2H), 7.73 (s, 1H), | |
| | | | | 7.53 (s, 1H), 7.30 (t, J = 8.9 Hz, 1H), | |
| | | | | 3.79 (t, J = 5.3 Hz, 2H), 3.45 (t, J = 5.3 | |
| | | | | Hz, 2H), 3.26 (s, 2H), 2.70 (tt, J = 7.8, | |
| | | | | 5.0 Hz, 2H), 1.56-1.41 (m, 15H), 1.02- | |
| | | | | 0.88 (m, 4H). | |
| 201 | 4.68 | 539.85 | D | ¹H NMR (400 MHz, DMSO-d6) δ | 12 |
| | | | | 12.92 (s, 1H), 8.12 (td, J = 5.8, 2.7 | |
| | | | | Hz, 2H), 7.90 (q, J = 1.3 Hz, 1H), | |
| | | | | 7.87-7.80 (m, 1H), 7.71 (d, J = 2.1 | |
| | | | | Hz, 1H), 7.49 (d, J = 1.9 Hz, 1H), | |
| | | | | 7.30 (td, J = 9.0, 2.2 Hz, 2H), 3.71 | |
| | | | | (t, J = 5.3 Hz, 2H), 3.33 (d, J = 4.8 | |
| | | | | Hz, 2H), 3.09 (d, J = 2.2 Hz, 2H), | |
| | | | | 1.60-1.31 (m, 15H). | |
| 202 | 1.51 | 424.29 | C | ¹H NMR (400 MHz, DMSO-d6) δ 8.22- | N/A |
| | | | | 8.10 (m, 3H), 7.85 (s, 1H), 7.54 (s, | |
| | | | | 1H), 7.35-7.25 (m, 2H), 3.70 (dd, J = | |
| | | | | 6.2, 3.5 Hz, 2H), 3.44-3.33 (m, 2H), | |
| | | | | 3.14 (q, J = 7.1 Hz, 1H), 1.78 (ddq, J = | |
| | | | | 20.5, 13.6, 7.2, 6.8 Hz, 2H), 1.66 (d, | |
| | | | | J = 1.8 Hz, 6H), 1.37 (d, J = 6.9 Hz, | |
| | | | | 3H), 0.81 (t, J = 7.4 Hz, 3H). | |
| 203 | 1.71 | 436.61 | C | ¹H NMR (400 MHz, DMSO-d6) δ 8.19- | 16 |
| | | | | 8.10 (m, 3H), 7.84 (s, 1H), 7.53 (s, | |
| | | | | 1H), 7.34-7.25 (m. 2H), 3.71 (t, J = | |
| | | | | 4.8 Hz, 2H), 3.43 (q, J = 8.3, 7.8 Hz, | |
| | | | | 1H), 3.38-3.32 (m, 2H), 2.13 (q, J = | |
| | | | | 9.3, 8.8 Hz, 2H), 1.90-1.78 (m, 4H), | |
| | | | | 1.76-1.68 (m, 2H), 1.66 (s, 6H). | |
| 204 | 1.54 | 581.48 | C | ¹H NMR (400 MHz, DMSO-d6) δ 8.27- | 3 |
| | | | | 8.16 (m, 2H), 8.04 (d, J = 2.5 Hz, 1H), | |
| | | | | 7.62 (d, J = 9.9 Hz, 1H), 7.39-7.30 | |
| | | | | (m, 2H), 4.96 (d, J = 16.8 Hz, 1H), | |
| | | | | 3.92-3.41 (m, 10H), 2.85 (h, J = 8.6 | |
| | | | | Hz, 1H), 2.11 (dt, J = 30.6, 9.8 Hz, | |
| | | | | 4H), 1.48 (s, 3H), 1.45 (s, 3H), 1.25 | |
| | | | | (d, J = 12.7 Hz, 3H). | |
| 205 | 1.7 | 635.43 | C | ¹H NMR (400 MHz, DMSO-d6) δ 8.26- | 3 |
| | | | | 8.19 (m, 2H), 8.04 (d, J = 2.2 Hz, 1H), | |
| | | | | 7.63 (d, J = 11.0 Hz, 1H), 7.39-7.30 | |
| | | | | (m, 2H), 6.56 (d, J = 19.3 Hz, 1H), | |
| | | | | 3.94-3.44 (m, 10H), 3.09-2.92 (m, 1H), | |
| | | | | 2.69-2.52 (m, 2H), 2.43-2.33 (m, 2H), | |
| | | | | 1.49 (s, 3H), 1.46 (s, 3H). | |
| 206 | 3.54 | 438.08 | A | ¹H NMR (400 MHz, DMSO-d6) δ 8.17 | 14 |
| | | | | (dd, J = 8.9, 5.5 Hz, 2H), 7.76 (s, 1H), | |
| | | | | 7.59 (s, 1H), 7.33 (t, J = 8.9 Hz, 2H), | |
| | | | | 3.83 (dd, J = 6.9, 3.2 Hz, 2H), 3.59- | |
| | | | | 3.52 (m, 2H), 2.94 (s, 3H), 1.71 (s, | |
| | | | | 6H), 1.53 (s, 9H). | |
| 207 | 4.58 | 564.59 | D | ¹H NMR (400 MHz, DMSO-d6) δ | 13 |
| | | | | 12.04 (s, 1H), 8.14 (ddt, J = 7.8, 5.5, | |
| | | | | 2.3 Hz, 2H), 7.72 (d, J = 3.4 Hz, 1H), | |
| | | | | 7.49 (d, J = 16.1 Hz, 1H), 7.30 (td, J = | |
| | | | | 8.9, 1.7 Hz, 2H), 3.97-3.84 (m, 2H), | |
| | | | | 3.84-3.72 (m, 1H), 3.65 (d, J = 28.5 | |
| | | | | Hz, 2H), 3.53-3.41 (m, 1H), 2.21-2.08 | |
| | | | | (m, 1H), 1.95-1.80 (m, 2H), 1.80-1.60 | |
| | | | | (m, 2H), 1.57-1.21 (m, 20H). | |
| 208 | 4.72 | 564.63 | D | ¹H NMR (400 MHz, DMSO-d6) δ | 13 |
| | | | | 12.13 (s, 1H), 8.13 (ddd, J = 8.9, 5.5, | |
| | | | | 1.8 Hz, 2H), 7.71 (d, J = 3.0 Hz, 1H), | |
| | | | | 7.49 (d, J = 14.4 Hz, 1H), 7.30 (td, J = | |
| | | | | 8.8, 1.8 Hz, 2H), 3.97-3.84 (m, 2H), | |
| | | | | 3.79-3.71 (m, 1H), 3.63 (d, J = 24.6 | |
| | | | | Hz, 2H), 3.50-3.40 (m, 1H), 2.03-1.88 | |
| | | | | (m, 2H), 1.63-1.31 (m, 23H). | |
| 209 | 3.22 | 482.09 | A | ¹H NMR (400 MHz, DMSO-d6) δ | N/A |
| | | | | 12.82 (s, 1H), 8.17 (dd, J = 8.9, 5.5 Hz, | |
| | | | | 2H), 7.76 (s, 1H), 7.60 (s, 1H), 7.33 (t, | |
| | | | | J = 8.9 Hz, 2H), 4.10 (s, 2H), 3.88 (dd, | |
| | | | | J = 6.6, 3.2 Hz, 2H), 3.61 (dd, J = 6.4, | |
| | | | | 3.4 Hz, 2H), 1.72 (s, 6H), 1.53 (s, 9H). | |
| 210 | 3.68 | 482.13 | A | ¹H NMR (400 MHz, DMSO-d6) δ 8.17 | 14 |
| | | | | (dd, J = 8.9, 5.5 Hz, 2H), 7.76 (s, 1H), | |
| | | | | 7.59 (s, 1H), 7.33 (t, J = 8.9 Hz, 2H), | |
| | | | | 3.86-3.79 (m, 2H), 3.61 (dd, J = 5.8, | |
| | | | | 3.9 Hz, 2H), 3.55 (dd, J = 6.0, 4.2 Hz, | |
| | | | | 2H), 3.49 (td, J = 5.2, 1.3 Hz, 2H), | |
| | | | | 3.27 (s, 3H), 1.71 (s, 6H), 1.53 (s, 9H). | |
| 211 | 3.62 | 450.07 | A | ¹H NMR (400 MHz, DMSO-d6) δ 8.15 | 16 |
| | | | | (td, J = 5.6, 2.2 Hz, 3H), 7.84 (s, 1H), | |
| | | | | 7.54 (s, 1H), 7.35-7.22 (m, 2H), 3.72 | |
| | | | | (t, J = 4.7 Hz, 2H), 3.37 (dt, J = 6.6, | |
| | | | | 3.5 Hz, 2H), 3.04 (tt, J = 12.0, 3.3 Hz, | |
| | | | | 1H), 1.97-1.80 (m, 4H), 1.73 (td, J = | |
| | | | | 12.4, 3.2 Hz, 3H), 1.66 (s, 6H), 1.53- | |
| | | | | 1.24 (m, 3H). | |
| 212 | 3.28 | 486.01 | A | ¹H NMR (400 MHz, DMSO-d6) δ 8.24- | 16 |
| | | | | 8.16 (m, 3H), 7.91 (s, 1H), 7.59 (s, | |
| | | | | 1H), 7.36-7.27 (m, 2H), 3.78-3.71 (m, | |
| | | | | 2H), 3.40 (s, 2H), 3.29-3.21 (m, 1H), | |
| | | | | 2.24-1.97 (m, 8H), 1.69 (s, 6H). | |
| 213 | 1.71 | 544.43 | C | ¹H NMR (400 MHz, DMSO-d6) δ 8.18- | 3 |
| | | | | 8.09 (m, 2H), 7.72 (d, J = 1.5 Hz, 1H), | |
| | | | | 7.50 (d, J = 11.3 Hz, 1H), 7.35-7.25 | |
| | | | | (m, 2H), 3.98 (t, J = 5.5 Hz, 1H), 3.91 | |
| | | | | (t, J = 5.8 Hz, 1H), 3.73 (t, J = 5.7 Hz, | |
| | | | | 1H), 3.66 (d, J = 7.0 Hz, 2H), 3.51 (t, | |
| | | | | J = 5.8 Hz, 1H), 3.38-3.31 (m, 2H), | |
| | | | | 2.99 (d, J = 7.4 Hz, 3H), 2.80 (dt, J = | |
| | | | | 11.3, 7.6 Hz, 2H), 1.57-1.39 (m, 15H). | |
| 214 | 1.6 | 426.39 | F | ¹H NMR (400 MHz, DMSO-d6) δ 8.18- | 16 |
| | | | | 8.09 (m, 1H), 7.42 (s, 1H), 7.11 (s, | |
| | | | | 1H), 3.75-3.65 (m, 2H), 3.39-3.30 | |
| | | | | (m, 2H), 1.90-1.80 (m, 2H), 1.80- | |
| | | | | 1.69 (m, 4H), 1.65 (s, 6H), 1.63-1.47 | |
| | | | | (m, 6H), 1.41 (s, 9H). | |
| 215 | 1.48 | 398.38 | F | ¹H NMR (400 MHz, DMSO-d6) δ 8.14 | 16 |
| | | | | (s, 1H), 7.42 (s, 1H), 7.14 (s, 1H), | |
| | | | | 3.78-3.61 (m, 2H), 2.08-1.90 (m, 3H), | |
| | | | | 1.76 (s, 5H), 1.65 (s, 9H), 1.42 (s, 9H). | |
| 216 | 3.02 | 448.38 | A | ¹H NMR (400 MHz, DMSO-d6) δ 8.16 | 16 |
| | | | | (t, J = 3.2 Hz, 1H), 7.45 (s, 1H), 7.17 | |
| | | | | (s, 1H), 3.70 (t, J = 4.9 Hz, 2H), 3.38- | |
| | | | | 3.32 (m, 2H), 3.04-2.88 (m, 1H), | |
| | | | | 2.16-1.97 (m, 3H), 1.97-1.76 (m, 6H), | |
| | | | | 1.65 (s, 6H), 1.42 (s, 9H). | |
| 217 | 3.7 | 440.13 | A | ¹H NMR (400 MHz, DMSO-d6) δ 8.15 | 16 |
| | | | | (t, J = 3.2 Hz, 1H), 7.44 (s, 1H), 7.13 | |
| | | | | (s, 1H), 3.70 (t, J = 4.7 Hz, 2H), 3.38- | |
| | | | | 3.32 (m, 2H), 2.75-2.61 (m, 1H), 1.80- | |
| | | | | 1.67 (m, 2H), 1.65 (s, 7H), 1.53 (d, | |
| | | | | J = 10.8 Hz, 1H), 1.45 (s, 1H), 1.42 (s, | |
| | | | | 9H), 1.37-1.18 (m, 3H), 0.93 (d, J = | |
| | | | | 8.7 Hz, 6H). | |
| 218 | 1.87 | 481.39 | C | N/A | N/A |
| 219 | 1.59 | 569.13 | E | 1H NMR (400 MHz, DMSO-d6) δ 7.40 | 3 |
| | | | | (d, J = 5.5 Hz, 1H), 7.16 (d, J = 1.2 Hz, | |
| | | | | 1H), 7.09 (dq, J = 6.6, 4.9 Hz, 1H), | |
| | | | | 4.27 (d, J = 45.3 Hz, 2H), 3.97-3.87 | |
| | | | | (m, 2H), 3.83 (t, J = 5.4 Hz, 1H), 3.69 | |
| | | | | (d, J = 36.5 Hz, 2H), 3.54-3.48 (m, | |
| | | | | 1H), 3.02-2.91 (m, 1H), 2.60 (dd, J = | |
| | | | | 4.9, 3.4 Hz, 3H), 2.15-1.76 (m, 8H), | |
| | | | | 1.48 (d, J = 10.5 Hz, 6H), 1.43 (d, J = | |
| | | | | 1.7 Hz, 9H). | |
| 220 | 1.87 | 602.86 | E | 1H NMR (400 MHz, DMSO-d6) δ 7.39 | 3 |
| | | | | (d, J = 16.6 Hz, 1H), 7.16 (d, J = 4.1 | |
| | | | | Hz, 1H), 3.94-3.78 (m, 3H), 3.72 (d, | |
| | | | | J = 5.6 Hz, 1H), 3.70-3.51 (m, 3H), | |
| | | | | 3.49-3.38 (m, 2H), 3.03-2.88 (m, 1H), | |
| | | | | 2.16-1.76 (m, 10H), 1.60-1.52 (m, | |
| | | | | 2H), 1.52-1.45 (m, 4H), 1.45-1.39 | |
| | | | | (m, 11H), 1.34 (d, J = 4.6 Hz, 1H), | |
| | | | | 1.29-1.25 (m, 1H), 1.20 (s, 1H), 1.06 | |
| | | | | (d, J = 2.9 Hz, 3H). | |
| 221 | 1.8 | 543.54 | E | 1H NMR (400 MHz, DMSO-d6) δ 7.39 | 3 |
| | | | | (d, J = 7.9 Hz, 1H), 7.16 (d, J = 7.3 Hz, | |
| | | | | 1H), 3.97 (t, J = 5.8 Hz, 2H), 3.91- | |
| | | | | 3.83 (m, 1H), 3.77 (s, 1H), 3.65 (t, J = | |
| | | | | 5.7 Hz, 2H), 3.03-2.90 (m, 1H), 2.35 | |
| | | | | (d, J = 8.5 Hz, 3H), 2.18-2.04 (m, 2H), | |
| | | | | 2.04-1.77 (m, 6H), 1.51 (s, 2H), 1.44 | |
| | | | | (d, J = 3.0 Hz, 9H), 1.39 (s, 4H). | |
| 222 | 1.74 | 569.44 | E | 1H NMR (400 MHz, DMSO-d6) δ 7.40 | 3 |
| | | | | (d, J = 10.9 Hz, 1H), 7.22-7.13 (m, | |
| | | | | 2H), 3.99-3.81 (m, 4H), 3.69-3.56 | |
| | | | | (m, 3H), 3.49 (t, J = 5.7 Hz, 1H), 2.97 | |
| | | | | (t, J = 11.3 Hz, 1H), 2.91 (d, J = 4.3 | |
| | | | | Hz, 3H), 2.15-2.03 (m, 2H), 2.03- | |
| | | | | 1.79 (m, 6H), 1.47 (d, J = 11.7 Hz, | |
| | | | | 6H), 1.43 (d, J = 2.1 Hz, 9H). | |
| 223 | 1.76 | 542.47 | E | 1H NMR (400 MHz, DMSO-d6) δ | 3 |
| | | | | 12.88 (s, 1H), 7.39 (d, J = 7.4 Hz, 1H), | |
| | | | | 7.16 (d, J = 5.4 Hz, 1H), 6.35 (d, J = | |
| | | | | 7.9 Hz, 1H), 4.22 (s, 1H), 4.13-4.01 | |
| | | | | (m, 1H), 4.02-3.91 (m, 1H), 3.92- | |
| | | | | 3.80 (m, 1H), 3.75 (s, 1H), 3.67-3.54 | |
| | | | | (m, 1H), 3.05-2.91 (m, 1H), 2.25- | |
| | | | | 2.16 (m, 3H), 2.16-1.77 (m, 9H), 1.50 | |
| | | | | (s, 3H), 1.43 (d, J = 3.7 Hz, 9H), 1.40 | |
| | | | | (s, 4H). | |
| 224 | 1.89 | 600.2 | E | 1H NMR (400 MHz, DMSO-d6) δ 7.40 | 3 |
| | | | | (d, J = 16.0 Hz, 1H), 7.16 (d, J = 2.2 | |
| | | | | Hz, 1H), 3.86 (t, J = 5.7 Hz, 2H), 3.66- | |
| | | | | 3.54 (m, 2H), 3.54-3.38 (m, 2H), 3.08- | |
| | | | | 2.84 (m, 2H), 2.74-2.49 (m, 2H), | |
| | | | | 2.37 (t, J = 11.0 Hz, 2H), 2.16-1.73 | |
| | | | | (m, 8H), 1.47 (s, 3H), 1.43 (d, J = 5.8 | |
| | | | | Hz, 11H). | |
| 225 | 1.72 | 560.25 | E | 1H NMR (400 MHz, DMSO-d6) δ 7.39 | 3 |
| | | | | (d, J = 14.8 Hz, 1H), 7.16 (d, J = 2.1 | |
| | | | | Hz, 1H), 4.27 (dd, J = 6.2, 2.9 Hz, 1H), | |
| | | | | 3.93-3.81 (m, 2H), 3.81-3.76 (m, | |
| | | | | 1H), 3.72 (t, J = 5.4 Hz, 1H), 3.62 (d, | |
| | | | | J = 18.7 Hz, 2H), 3.47-3.37 (m, 1H), | |
| | | | | 2.97 (t, 1H), 2.17-1.53 (m, 12H), 1.48 | |
| | | | | (s, 4H), 1.43 (d, J = 1.4 Hz, 13H), 1.39- | |
| | | | | 1.28 (m, 3H). | |
| 226 | 1.7 | 583.21 | E | 1H NMR (400 MHz, DMSO-d6) δ 7.40 | 3 |
| | | | | (d, J = 9.5 Hz, 1H), 7.16 (d, J = 1.3 Hz, | |
| | | | | 1H), 6.91 (dd, J = 15.4, 5.0 Hz, 1H), | |
| | | | | 3.98-3.91 (m, 1H), 3.91-3.81 (m, | |
| | | | | 1H), 3.72-3.65 (m, 1H), 3.62 (d, J = | |
| | | | | 6.7 Hz, 2H), 3.51-3.45 (m, 1H), 3.24 | |
| | | | | (t, J = 7.3 Hz, 3H), 3.02-2.92 (m, 1H), | |
| | | | | 2.70 (dt, J = 15.3, 7.5 Hz, 2H), 2.54 | |
| | | | | (dd, J = 5.8, 5.0 Hz, 3H), 2.15-1.78 | |
| | | | | (m, 8H), 1.48 (d, J = 18.5 Hz, 5H), 1.43 | |
| | | | | (d, J = 3.0 Hz, 8H). | |
| 227 | 3.77 | 535.56 | A | 1H NMR (400 MHz, DMSO-d6) δ 7.39 | 3 |
| | | | | (d, J = 7.9 Hz, 1H), 7.12 (d, J = 7.3 Hz, | |
| | | | | 1H), 4.05-3.95 (m, 1H), 3.87 (s, 1H), | |
| | | | | 3.77 (s, 1H), 3.68-3.62 (m, 1H), 2.74- | |
| | | | | 2.62 (m, 1H), 2.35 (d, J = 8.5 Hz, 3H), | |
| | | | | 1.84-1.61 (m, 3H), 1.51 (s, 2H), 1.44 | |
| | | | | (s, 8H), 1.35-1.19 (m, 2H), 0.94 (d, J = | |
| | | | | 3.4 Hz, 3H), 0.92 (d, J = 2.3 Hz, 3H). | |
| 228 | 3.91 | 538.36 | A | 1H NMR (400 MHz, dmso) δ 7.38 (d, | 3 |
| | | | | J = 13.1 Hz, 1H), 7.13 (d, J = 2.3 Hz, | |
| | | | | 1H), 3.85 (s, 2H), 3.62-3.41 (m, 4H), | |
| | | | | 2.87-2.74 (m, 1H), 2.72-2.65 (m, | |
| | | | | 1H), 2.18-2.02 (m, 4H), 1.81-1.60 | |
| | | | | (m, 4H), 1.48-1.40 (m, 17H), 1.35- | |
| | | | | 1.22 (m, 5H), 0.93 (d, J = 9.1 Hz, 6H). | |
| 229 | 1.83 | 588.25 | E | 1H NMR (400 MHz, DMSO-d6) δ 7.39 | 3, 13 |
| | | | | (d, J = 16.0 Hz, 1H), 7.16 (d, J = 3.4 | |
| | | | | Hz, 1H), 3.85 (dd, J = 11.8, 6.1 Hz, | |
| | | | | 2H), 3.77-3.69 (m, 1H), 3.62 (d, J = | |
| | | | | 24.1 Hz, 2H), 3.42 (t, J = 5.8 Hz, 1H), | |
| | | | | 3.04-2.92 (m, 1H), 2.16-1.75 (m, | |
| | | | | 11H), 1.60-1.44 (m, 9H), 1.45-1.37 | |
| | | | | (m, 13H). | |
| 230 | 1.81 | 540.79 | E | 1H NMR (400 MHz, DMSO-d6) δ 8.89 | 3 |
| | | | | (dd, J = 12.9, 1.5 Hz, 1H), 8.76 (t, J = | |
| | | | | 2.5 Hz, 1H), 8.68 (ddd, J = 10.1, 2.6, | |
| | | | | 1.5 Hz, 1H), 7.40 (d, J = 13.5 Hz, 1H), | |
| | | | | 7.15 (d, J = 15.9 Hz, 1H), 4.00 (t, J = | |
| | | | | 5.8 Hz, 1H), 3.89-3.78 (m, 2H), 3.78- | |
| | | | | 3.63 (m, 3H), 2.97 (d, J = 11.8 Hz, 1H), | |
| | | | | 2.15-1.76 (m, 8H), 1.56 (s, 3H), 1.43 | |
| | | | | (d, J = 15.4 Hz, 9H), 1.34 (s, 4H). | |
| 231 | 1.73 | 540.51 | E | 1H NMR (400 MHz, DMSO-d6) δ 8.90 | 3 |
| | | | | (dd, J = 5.0, 3.1 Hz, 2H), 7.60 (td, J = | |
| | | | | 5.0, 3.0 Hz, 1H), 7.39 (d, J = 16.0 Hz, | |
| | | | | 1H), 7.15 (d, J = 18.6 Hz, 1H), 3.98 (t, | |
| | | | | J = 5.8 Hz, 1H), 3.84-3.75 (m, 2H), | |
| | | | | 3.69 (t, J = 5.8 Hz, 1H), 3.49-3.34 (m, | |
| | | | | 2H), 2.96 (d, J = 12.8 Hz, 1H), 2.18- | |
| | | | | 1.74 (m, 9H), 1.55 (s, 2H), 1.45 (s, 5H), | |
| | | | | 1.39 (s, 4H), 1.32 (s, 4H). | |
| 232 | 1.71 | 568.46 | E | 1H NMR (400 MHz, DMSO-d6) δ 7.40 | 3 |
| | | | | (d, J = 12.3 Hz, 1H), 7.16 (d, J = 1.4 | |
| | | | | Hz, 1H), 3.94 (t, J = 5.5 Hz, 1H), 3.87 | |
| | | | | (t, J = 5.9 Hz, 1H), 3.70 (t, J = 5.6 Hz, | |
| | | | | 1H), 3.64 (d, J = 6.6 Hz, 2H), 3.48 (t, | |
| | | | | J = 5.7 Hz, 1H), 2.99 (d, J = 7.3 Hz, | |
| | | | | 4H), 2.87-2.66 (m, 2H), 2.17-1.96 (m, | |
| | | | | 3H), 1.97-1.76 (m, 5H), 1.50 (s, 3H), | |
| | | | | 1.45 (s, 3H), 1.43 (d, J = 3.0 Hz, 9H). | |
| 233 | 1.87 | 438.41 | E | 1H NMR (400 MHz, DMSO-d6) δ 8.25- | 16 |
| | | | | 8.03 (m, 3H), 7.84 (s, 1H), 7.54 (s, | |
| | | | | 1H), 7.42-7.16 (m, 2H), 3.74-3.64 | |
| | | | | (m, 2H), 3.35 (dt, J = 6.8, 3.5 Hz, 2H), | |
| | | | | 3.00-2.78 (m, 1H), 1.92-1.71 (m, 4H), | |
| | | | | 1.66 (s, 6H), 0.75 (t, J = 7.4 Hz, 6H). | |
| 234 | 1.61 | 546.45 | E | 1H NMR (400 MHz, cdcl3) δ 7.34 (s, | 3 |
| | | | | 1H), 7.06 (2s, 1H), 4.00-3.85 (m, 2H), | |
| | | | | 3.80-3.69 (m, 2H), 3.65 (t, J = 5.6 Hz, | |
| | | | | 1H), 3.47 (s, 1H), 2.99-2.71 (m, 2H), | |
| | | | | 2.44-2.32 (m, 4H), 2.30-2.17 (m, | |
| | | | | 2H), 2.11-1.76 (m, 6H), 1.73-1.55 | |
| | | | | (m, 7H), 1.50 (2s, 9H), 1.41 (2s, 3H). | |
| | | | | Signals doubled due to amide rotamers, | |
| 235 | 3.93 | 491.47 | A | 1H NMR (400 MHz, DMSO-d6) δ | 21 |
| | | | | 12.57 (s, 1H), 8.20-8.10 (m, 2H), 7.77- | |
| | | | | 7.66 (m, 2H), 7.61 (s, 1H), 7.35-7.26 | |
| | | | | (m, 2H), 6.69 (dd, 1H), 4.16-4.08 (m, | |
| | | | | 2H), 4.02-3.95 (m, 2H), 1.78 (s, 6H), | |
| | | | | 1.51 (s, 9H). | |
| 236 | 1.9 | 454.38 | A | 1H NMR (400 MHz, DMSO-d6) δ 8.15- | 14 |
| | | | | 8.10 (m, 2H), 7.76 (s, 1H), 7.57 (s, | |
| | | | | 1H), 7.56-7.51 (m, 2H), 3.85-3.76 | |
| | | | | (m, 2H), 3.58-3.47 (m, 2H), 2.91 (s, | |
| | | | | 3H), 1.67 (s, 6H), 1.50 (s, 9H). | |
| 237 | 4.24 | 475.65 | A | 1H NMR (400 MHz, DMSO-d6) δ 8.15 | 14 |
| | | | | (td, J = 1.8, 0.6 Hz, 1H), 8.07 (dt, J = | |
| | | | | 7.3, 1.7 Hz, 1H), 7.79 (s, 1H), 7.58 (s, | |
| | | | | 1H), 7.55-7.46 (m, 2H), 3.85-3.76 | |
| | | | | (m, 2H), 3.59-3.47 (m, 2H), 1.68 (s, | |
| | | | | 6H), 1.51 (s, 9H). | |
| 238 | 1.9 | 457.37 | A | 1H NMR (400 MHz, DMSO-d6) δ 8.15 | 14 |
| | | | | (dd, J = 11.0, 2.1 Hz, 1H), 8.01 (ddd, | |
| | | | | J = 8.5, 2.1, 0.7 Hz, 1H), 7.82 (s, 1H), | |
| | | | | 7.69 (dd, J = 8.5, 7.8 Hz, 1H), 7.57 (s, | |
| | | | | 1H), 3.85-3.76 (m, 2H), 3.57-3.47 | |
| | | | | (m, 2H), 1.67 (s, 6H), 1.50 (s, 9H). | |
| 239 | 1.92 | 475.34 | A | 1H NMR (400 MHz, DMSO-d6) δ 8.32 | 14 |
| | | | | (dd, J = 7.3, 2.3 Hz, 1H), 8.14 (ddd, J = | |
| | | | | 8.7, 4.7, 2.3 Hz, 1H), 7.79 (s, 1H), 7.57 | |
| | | | | (s, 1H), 7.56-7.49 (m, 1H), 3.85-3.75 | |
| | | | | (m, 2H), 3.58-3.48 (m, 2H), 1.67 (s, | |
| | | | | 6H), 1.50 (s, 9H). | |
| 240 | 4.13 | 457.45 | A | 1H NMR (400 MHz, DMSO-d6) δ 8.16- | 14 |
| | | | | 8.10 (m, 2H), 7.76 (s, 1H), 7.57 (s, | |
| | | | | 1H), 7.56-7.51 (m, 2H), 3.84-3.76 (m, | |
| | | | | 2H), 3.56-3.48 (m, 2H), 1.67 (s, 6H), | |
| | | | | 1.50 (s, 9H). | |
| 241 | 1.94 | 472.3 | A | 1H NMR (400 MHz, DMSO-d6) δ 8.15 | 14 |
| | | | | (dd, J = 11.0, 2.1 Hz, 1H), 8.01 (ddd, | |
| | | | | J = 8.4, 2.1, 0.7 Hz, 1H), 7.82 (s, 1H), | |
| | | | | 7.69 (dd, J = 8.5, 7.8 Hz, 1H), 7.57 (s, | |
| | | | | 1H), 3.86-3.76 (m, 2H), 3.57-3.47 | |
| | | | | (m, 2H), 2.91 (s, 3H), 1.67 (s, 6H), | |
| | | | | 1.50 (s, 9H). | |
| 242 | 1.91 | 454.34 | E | 1H NMR (400 MHz, DMSO-d6) δ 8.16 | 14 |
| | | | | (td, J = 1.8, 0.6 Hz, 1H), 8.07 (dt, J = | |
| | | | | 7.3, 1.7 Hz, 1H), 7.79 (s, 1H), 7.58 (s, | |
| | | | | 1H), 7.55-7.45 (m, 2H), 3.86-3.76 | |
| | | | | (m, 2H), 3.57-3.48 (m, 2H), 2.91 (s, | |
| | | | | 3H), 1.68 (s, 6H), 1.51 (s, 9H). | |
| 243 | 1.93 | 472.3 | E | 1H NMR (400 MHz, DMSO-d6) δ 8.32 | 14 |
| | | | | (dd, J = 7.3, 2.3 Hz, 1H), 8.14 (ddd, J = | |
| | | | | 8.7, 4.7, 2.3 Hz, 1H), 7.79 (s, 1H), 7.57 | |
| | | | | (s, 1H), 7.52 (t, J = 9.0 Hz, 1H), 3.85- | |
| | | | | 3.76 (m, 2H), 3.57-3.48 (m, 2H), 2.91 | |
| | | | | (s, 3H), 1.67 (s, 6H), 1.50 (s, 9H). | |
| 244 | 1.86 | 440.32 | E | 1H NMR (400 MHz, DMSO-d6) δ 8.18 | 3 |
| | | | | (t, J = 3.1 Hz, 1H), 8.15 (td, J = 1.8, | |
| | | | | 0.6 Hz, 1H), 8.07 (dt, J = 7.2, 1.7 Hz, | |
| | | | | 1H), 7.79 (s, 1H), 7.58 (s, 1H), 7.55- | |
| | | | | 7.45 (m, 2H), 3.79-3.69 (m, 2H), 3.36 | |
| | | | | (dt, J = 5.4, 3.6 Hz, 2H), 1.67 (s, 6H), | |
| | | | | 1.50 (s, 9H). | |
| 245 | 1.75 | 424.64 | E | 1H NMR (400 MHz, DMSO-d6) δ 8.20- | 16 |
| | | | | 8.11 (m, 3H), 7.86 (d, J = 3.9 Hz, 1H), | |
| | | | | 7.55 (d, J = 7.5 Hz, 1H), 7.34-7.25 | |
| | | | | (m, 2H), 3.74-3.67 (m, 2H), 3.41-3.33 | |
| | | | | (m, 2H), 3.14 (q, J = 7.1 Hz, 1H), | |
| | | | | 1.78 (qt, J = 13.7, 7.2 Hz, 2H), 1.70- | |
| | | | | 1.61 (m, 6H), 1.37 (dd, J = 6.9, 4.0 Hz, | |
| | | | | 3H), 0.87-0.74 (m, 3H). | |
| 246 | 1.75 | 424.64 | E | 1H NMR (400 MHz, DMSO-d6) δ 8.24- | 16 |
| | | | | 8.11 (m, 3H), 7.86 (d, J = 4.0 Hz, 1H), | |
| | | | | 7.55 (d, J = 7.5 Hz, 1H), 7.36-7.22 | |
| | | | | (m, 2H), 3.70 (dd, J = 6.4, 3.5 Hz, 2H), | |
| | | | | 3.40-3.32 (m, 2H), 3.13 (p, J = 7.0 | |
| | | | | Hz, 1H), 1.89-1.70 (m, 3H), 1.71- | |
| | | | | 1.62 (m, 6H), 1.37 (dd, J = 6.9, 4.0 Hz, | |
| | | | | 3H), 0.81 (td, J = 7.2, 2.9 Hz, 4H). | |
| 247 | 3.7 | 505.42 | A | 1H NMR (400 MHz, DMSO-d6) δ | 21 |
| | | | | 12.47 (s, 1H), 8.19-8.10 (m, 2H), 7.73 | |
| | | | | (s, 1H), 7.61 (s, 1H), 7.51-7.46 (m, | |
| | | | | 1H), 7.35-7.26 (m, 2H), 4.02-3.97 | |
| | | | | (m, 2H), 3.93-3.87 (m, 2H), 1.84- | |
| | | | | 1.78 (m, 3H), 1.79 (s, 6H), 1.49 (s, 9H). | |
| 248 | 3.73 | 504.39 | A | 1H NMR (400 MHz, DMSO-d6) δ | 21 |
| | | | | 12.24 (s, 1H), 8.19-8.10 (m, 2H), 7.74 | |
| | | | | (s, 1H), 7.60 (s, 1H), 7.35-7.26 (m, | |
| | | | | 2H), 6.45 (s, 1H), 4.09-4.03 (m, 2H), | |
| | | | | 3.99-3.92 (m, 2H), 2.22-2.17 (m, | |
| | | | | 3H), 1.77 (s, 6H), 1.51 (s, 9H). | |
| 249 | 3.73 | 452.41 | A | 1H NMR (400 MHz, DMSO-d6) δ 8.46 | 16 |
| | | | | (s, 1H), 8.17 (dd, J = 6.0, 2.7 Hz, 2H), | |
| | | | | 7.85 (s, 1H), 7.54 (s, 1H), 7.30 (t, J = | |
| | | | | 8.9 Hz, 2H), 3.68 (t, J = 4.9 Hz, 2H), | |
| | | | | 3.39-3.35 (m, 2H), 3.10-2.95 (m, | |
| | | | | 2H), 1.85-1.70 (m, 4H), 1.66 (s, 6H), | |
| | | | | 1.22-1.08 (m, 2H), 0.77 (dt, J = 27.0, | |
| | | | | 7.3 Hz, 6H). | |
| 250 | 3.57 | 504.44 | A | 1H NMR (400 MHz, dmso) δ 8.14 (dd, | 1 |
| | | | | J = 8.7, 5.5 Hz, 2H), 7.73 (s, 1H), 7.60 | |
| | | | | (s, 1H), 7.30 (t, J = 8.8 Hz, 2H), 4.00- | |
| | | | | 3.90 (m, 2H), 3.81-3.74 (m, 2H), 2.05 | |
| | | | | (s, 3H), 1.77 (s, 6H), 1.49 (s, 9H). | |
| 251 | 2.96 | 480.73 | A | 1H NMR (400 MHz, DMSO-d6) δ 8.13 (t, J = 3.2 Hz, 1H), 8.08-7.98 (m, 2H), 7.56-7.51 (m, 2H), 7.47-7.37 (m, 2H), 4.74 (d, J = 6.3 Hz, 0H), 4.04 (s, 1H), 3.89-3.71 (m, 2H), 3.60-3.51 (m, 0H), 2.86-2.63 (m, 1H), 2.11- 1.90 (m, 2H), 1.75-1.55 (m, 10H), 1.52-1.36 (m, 2H), 1.13 (s, 3H). | 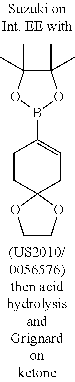 |
| 252 | 3.58 | 593.2 | A | 1H NMR (400 MHz, CD3OD) δ 8.82 | 3, 11 |
| | | | | (d, J = 6.6 Hz, 1H), 8.29 (t, J = 9.2 Hz, | |
| | | | | 1H), 8.15 (t, J = 9.7 Hz, 1H), 8.04- | |
| | | | | 7.71 (m, 3H), 7.63 (t, J = 8.1 Hz, 1H), | |
| | | | | 7.46 (d, J = 16.3 Hz, 1H), 4.20 (d, J = | |
| | | | | 6.0 Hz, 1H), 4.09-3.88 (m, 3H), 3.72 | |
| | | | | (d, J = 16.3 Hz, 2H), 1.75 (s, 2H), 1.64 | |
| | | | | (s, 6H), 1.57 (s, 4H), 1.51 (s, 3H). | |
| 253 | 3.53 | 741.22 | A | 1H NMR (400 MHz, DMSO-d6) δ 8.18 | 3, 11 |
| | | | | (dd, J = 11.0, 2.1 Hz, 1H), 8.05 (dd, J = | |
| | | | | 8.5, 2.0 Hz, 1H), 7.84 (s, 1H), 7.74 (dt, | |
| | | | | J = 11.4, 7.9 Hz, 2H), 7.66-7.49 (m, | |
| | | | | 1H), 7.40 (d, J = 7.3 Hz, 1H), 6.98 (d, | |
| | | | | J = 8.6 Hz, 1H), 5.61 (d, J = 6.8 Hz, | |
| | | | | 1H), 5.51-5.42 (m, 1H), 5.33-5.24 (m, | |
| | | | | 1H), 4.16-4.03 (m, 2H), 4.01-3.88 (m, | |
| | | | | 2H), 3.79 (d, J = 8.9 Hz, 1H), 3.74- | |
| | | | | 3.62 (m, 2H), 1.55 (d, J = 4.0 Hz, 15H). | |
| 254 | 4.06 | 568.85 | A | 1H NMR (400 MHz, CDCl3) δ 13.56 | 3, 11 |
| | | | | (s, 1H), 7.50 (d, J = 4.7 Hz, 1H), 7.15 | |
| | | | | (d, J = 2.6 Hz, 1H), 5.01 (ddd, J = | |
| | | | | 22.6, 7.6, 5.3 Hz, 1H), 4.80-4.65 (m, | |
| | | | | 1H), 4.15-3.62 (m, 5H), 3.54 (q, J = | |
| | | | | 13.7 Hz, 1H), 2.78 (d, J = 18.4 Hz, | |
| | | | | 1H), 2.59-2.38 (m, 2H), 2.29 (ddd, | |
| | | | | J = 12.6, 10.3, 4.6 Hz, 1H), 2.06 | |
| | | | | (dh, J = 12.2, 6.1 Hz, 1H), 1.89- | |
| | | | | 1.81 (m, 4H), 1.71-1.62 (m, 6H), | |
| | | | | 1.54 (d, J = 2.0 Hz, 11H), 1.41 (dt, | |
| | | | | J = 12.9, 8.5 Hz, 2H), 1.05 (s, 3H), | |
| | | | | 1.00 (d, J = 1.0 Hz, 3H). | |
| 255 | 4.02 | 586.25 | A | 1H NMR (400 MHz, CDCl3) δ 13.50 | 3, 11 |
| | | | | (s, 1H), 7.87 (dd, J = 10.3, 1.9 Hz, 1H), | |
| | | | | 7.79 (d, J = 8.4 Hz, 1H), 7.63 (d, J = | |
| | | | | 1.5 Hz, 1H), 7.54 (td, J = 6.8, 6.1, 3.5 | |
| | | | | Hz, 2H), 5.19-4.94 (m, 1H), 4.74 (dt, | |
| | | | | J = 7.5, 5.4 Hz, 1H), 4.18-3.66 (m, | |
| | | | | 5H), 3.56 (q, J = 13.8 Hz, 1H), 2.54- | |
| | | | | 2.38 (m, 2H), 2.33-2.23 (m, 1H), 2.06 | |
| | | | | (dt, J = 11.1, 4.9 Hz, 1H), 1.74-1.59 | |
| | | | | (m, 15H). | |
| 256 | 3.9 | 568.25 | A | 1H NMR (400 MHz, CDCl3) δ 13.48 | 3, 11 |
| | | | | (s, 1H), 8.03 (d, J = 7.7 Hz, 2H), 7.83- | |
| | | | | 7.67 (m, 2H), 7.61-7.47 (m, 2H), 5.03 | |
| | | | | (dd, J = 16.5, 10.7 Hz, 1H), 4.83-4.64 | |
| | | | | (m, 1H), 4.22-3.65 (m, 5H), 3.56 (q, | |
| | | | | J = 13.7 Hz, 1H), 2.56-2.39 (m, 2H), | |
| | | | | 2.28 (dd, J = 12.0, 6.4 Hz, 1H), 2.07 | |
| | | | | (d, J = 8.6 Hz, 1H), 1.72-1.61 (m, 15H). | |
| 257 | 3.26 | 521.64 | A | 1H NMR (400 MHz, DMSO-d6) δ 8.20- | 22 |
| | | | | 8.12 (m, 3H), 8.03 (dd, J = 8.4, 1.9 | |
| | | | | Hz, 1H), 7.83 (s, 1H), 7.71 (t, J = 8.2 | |
| | | | | Hz, 1H), 7.55 (s, 1H), 6.71 (d, J = 6.5 | |
| | | | | Hz, 2H), 4.10 (t, J = 5.4 Hz, 2H), 3.70 | |
| | | | | (s, 2H), 3.51 (t, J = 5.5 Hz, 2H), 1.54 | |
| | | | | (s, 9H), 1.52 (s, 6H). | |
| 258 | 3.98 | 493.68 | A | 1H NMR (400 MHz, DMSO-d6) δ | 3, 21 |
| | | | | 12.31 (s, 1H), 7.42 (s, 1H), 7.28 (broad | |
| | | | | s, 2H), 7.15 (s, 1H), 3.77-3.65 (m, | |
| | | | | 2H), 3.09-2.97 (m, 2H), 2.90 (s, 2H), | |
| | | | | 2.77-2.61 (m, 1H), 1.83-1.62 (m, | |
| | | | | 4H), 1.51 (s, 6H), 1.49-1.46 (m, 1H), | |
| | | | | 1.44 (s, 10H), 1.33 (td, J = 13.5, 4.4 | |
| | | | | Hz, 2H), 0.97 (s, 3H), 0.94 (s, 3H). | |
| 259 | 4.29 | 510.67 | A | 1H NMR (400 MHz, DMSO-d6) δ | 3, 21 |
| | | | | 12.31 (s, 1H), 8.16 (dd, J = 11.0, 2.0 | |
| | | | | Hz, 1H), 8.03 (dd, J = 8.4, 1.9 Hz, 1H), | |
| | | | | 7.83 (s, 1H), 7.71 (t, J = 8.1 Hz, 1H), | |
| | | | | 7.54 (s, 1H), 7.29 (s, 2H), 3.81-3.66 | |
| | | | | (m, 2H), 3.13-3.02 (m, 2H), 2.93 (s, | |
| | | | | 2H), 1.53 (s, 6H), 1.52 (s, 9H). | |
| 260 | 3.77 | 524.2 | A | 1H NMR (400 MHz, DMSO-d6) δ | 3, 21 |
| | | | | 12.81 (s, 1H), 8.17 (dd, J = 11.0, 2.0 | |
| | | | | Hz, 1H), 8.04 (dd, J = 8.4, 1.8 Hz, 1H), | |
| | | | | 7.87 (broad s, 2H), 7.85 (s, 1H), 7.71 | |
| | | | | (t, J = 8.1 Hz, 1H), 7.64 (s, 1H), 4.04- | |
| | | | | 3.98 (m, 2H), 3.98-3.91 (m, 2H), 1.79 | |
| | | | | (s, 6H), 1.54 (s, 9H). | |
| 261 | 3.18 | 567.18 | A | 1H NMR (400 MHz, DMSO-d6) δ 8.15 | 22A, 11 |
| | | | | (dd, J = 11.0, 2.1 Hz, 1H), 8.06-7.99 | |
| | | | | (m, 2H), 7.82 (s, 1H), 7.69 (t, J = 8.1 | |
| | | | | Hz, 1H), 7.56 (s, 1H), 7.34 (s, 1H), | |
| | | | | 7.01 (s, 1H), 4.15 (t, J = 5.4 Hz, 2H), | |
| | | | | 3.92 (s, 2H), 3.71 (t, J = 5.6 Hz, 2H), | |
| | | | | 1.53 (s, 6H), 1.52 (s, 9H). | |
| 262 | 3.23 | 503.53 | A | 1H NMR (400 MHz, DMSO-d6) δ 8.21- | 22A |
| | | | | 8.06 (m, 4H), 7.77 (s, 1H), 7.60-7.49 | |
| | | | | (m, 3H), 6.68 (d, J = 6.5 Hz, 2H), 4.09 | |
| | | | | (t, J = 5.4 Hz, 2H), 3.68 (s, 2H), 3.50 (t, | |
| | | | | J = 5.6 Hz, 2H), 1.53 (s, 9H), 1.52 | |
| | | | | (s, 6H). | |
| 263 | 3.38 | 518.42 | A | 1H NMR (400 MHz, DMSO-d6) δ 8.27- | 22A |
| | | | | 8.01 (m, 2H), 7.78 (s, 1H), 7.58-7.54 | |
| | | | | (m, 2H), 7.54 (s, 1H), 7.29 (d, J = 9.4 | |
| | | | | Hz, 1H), 7.06 (d, J = 9.3 Hz, 1H), 4.22- | |
| | | | | 4.00 (m, 2H), 3.91 (s, 2H), 3.73-3.53 | |
| | | | | (m, 2H), 2.43 (s, 3H), 1.53 (s, 9H), | |
| | | | | 1.53 (s, 6H). | |
| 264 | 4.83 | 518.66 | A | 1H NMR (400 MHz, DMSO-d6) δ 8.23- | 22A |
| | | | | 8.11 (m, 1H), 8.05 (d, J = 1.5 Hz, 1H), | |
| | | | | 7.96 (dd, J = 1.4, 0.6 Hz, 1H), 7.77 (s, | |
| | | | | 1H), 7.59-7.54 (m, 2H), 7.54 (s, 1H), | |
| | | | | 4.19-4.01 (m, 2H), 3.88 (s, 2H), 3.64 | |
| | | | | (t, J = 5.7 Hz, 2H), 2.31 (s, 3H), 1.53 | |
| | | | | (s, 9H), 1.52 (s, 6H). | |
| 265 | 3.36 | 518.52 | A | 1H NMR (400 MHz, DMSO-d6) δ 8.44 | 22A |
| | | | | (s, 1H), 8.19-8.11 (m, 2H), 8.08 (s, | |
| | | | | 1H), 7.77 (s, 1H), 7.61-7.49 (m, 3H), | |
| | | | | 4.12-3.92 (m, 2H), 3.91-3.70 (m, 4H), | |
| | | | | 2.29 (s, 3H), 1.56 (s, 6H), 1.53 (s, 9H). | |
| 266 | 4.97 | 519.45 | A | 1H NMR (400 MHz, DMSO-d6) δ 8.19- | 22A |
| | | | | 8.11 (m, 2H), 8.07 (dd, J = 2.6, 0.5 Hz, | |
| | | | | 1H), 8.02 (d, J = 2.7 Hz, 1H), 7.78 (s, | |
| | | | | 1H), 7.60-7.51 (m, 3H), 3.96-3.84 (m, | |
| | | | | 2H), 3.56-3.49 (m, 2H), 3.39 (s, 2H), | |
| | | | | 2.52 (s, 3H), 1.58 (s, 6H), 1.53 (s, 9H). | |
| 267 | 3.33 | 522.39 | A | 1H NMR (400 MHz, DMSO-d6) δ 8.17 | 22B |
| | | | | (dd + broad s, J = 11.0, 2.0 Hz, 2H), | |
| | | | | 8.03 (dd, J = 8.4, 1.7 Hz, 1H), 7.89 | |
| | | | | (broad s, 1H), 7.83 (s, 1H), 7.76-7.64 | |
| | | | | (m, 1H), 7.55 (s, 1H), 7.26-7.05 (m, | |
| | | | | 2H), 4.05 (t, J = 5.4 Hz, 2H), 3.61 (s, | |
| | | | | 2H), 3.49 (t, J = 5.4 Hz, 2H), 1.53 | |
| | | | | (s, 15H). | |
| 268 | 4.76 | 579.3 | A | 1H NMR (400 MHz, DMSO-d6) δ 8.22- | 22A |
| | | | | 8.10 (m, 1H), 8.08-7.98 (m, 1H), 7.81 | |
| | | | | (s, 1H), 7.74-7.63 (m, 2H), 7.53 (s, 1H), | |
| | | | | 7.27 (dd, J = 7.2, 0.6 Hz, 1H), 6.89 | |
| | | | | (d, J = 8.6 Hz, 1H), 4.13-4.01 (m, 2H), | |
| | | | | 3.90 (s, 2H), 3.80 (s, 3H), 3.72-3.55 | |
| | | | | (m, 2H), 1.56-1.35 (m, 18H). | |
| 269 | 2.96 | 505.25 | A | 1H NMR (400 MHz, DMSO-d6) δ 8.20- | 22B |
| | | | | 8.06 (m, 3H), 7.89 (dd, J = 4.3, 1.3 | |
| | | | | Hz, 1H), 7.77 (s, 1H), 7.58-7.52 (m, | |
| | | | | 3H), 7.19 (dd, J = 8.2, 4.3 Hz, 1H), | |
| | | | | 7.15 (ddd, J = 8.6, 2.8, 1.5 Hz, 1H), | |
| | | | | 4.05 (t, J = 5.5 Hz, 2H), 3.61 (s, 2H), | |
| | | | | 3.49 (t, J = 5.5 Hz, 2H), 1.54 (s, 6H), | |
| | | | | 1.53 (s, 9H). | |
| 270 | 3.49 | 504.37 | A | 1H NMR (400 MHz, DMSO-d6) δ 8.08 | 1 |
| | | | | (ddd, J = 4.9, 1.9, 0.8 Hz, 1H), 7.61- | |
| | | | | 7.45 (m, 1H), 7.42 (s, 1H), 7.15 (s, | |
| | | | | 1H), 6.66-6.50 (m, 2H), 4.11-3.98 | |
| | | | | (m, 2H), 3.88 (s, 2H), 3.56 (t, J = 5.6 | |
| | | | | Hz, 2H), 2.70 (tt, J = 11.9, 4.0 Hz, 1H), | |
| | | | | 1.84-1.62 (m, 4H), 1.50 (s, 6H), 1.49- | |
| | | | | 1.47 (m, 2H), 1.46 (s, 9H), 1.33 (td, | |
| | | | | J = 13.1, 3.9 Hz, 2H), 0.97 (s, 3H), | |
| | | | | 0.94 (s, 3H). | |
| 271 | 3.45 | 523.27 | A | 1H NMR (400 MHz, DMSO-d6) δ 8.17 | 1 |
| | | | | (dd, J = 11.0, 2.1 Hz, 1H), 8.09 (ddd, | |
| | | | | J = 4.9, 2.0, 0.8 Hz, 1H), 8.07-7.95 (m, | |
| | | | | 1H), 7.83 (s, 1H), 7.78-7.64 (m, 1H), | |
| | | | | 7.61-7.50 (m, 2H), 6.66-6.51 (m, 2H), | |
| | | | | 4.15-4.01 (m, 2H), 3.90 (s, 2H), 3.58 | |
| | | | | (t, J = 5.6 Hz, 2H), 1.53 (s, 10H), 1.51 | |
| | | | | (s, 6H). | |
| 272 | 3.42 | 503.67 | A | 1H NMR (400 MHz, DMSO-d6) δ 8.21- | 1 |
| | | | | 8.12 (m, 2H), 8.09 (ddd, J = 4.9, 1.9, | |
| | | | | 0.7 Hz, 1H), 7.77 (s, 1H), 7.61-7.47 | |
| | | | | (m, 4H), 6.66-6.54 (m, 2H), 4.14-4.01 | |
| | | | | (m, 2H), 3.90 (s, 2H), 3.58 (t, J = 5.6 | |
| | | | | Hz, 2H), 1.53 (s, 9H), 1.52 (s, 6H). | |
| 273 | 4.04 | 493.4 | A | 1H NMR (400 MHz, DMSO-d6) δ | 1, 23 |
| | | | | 12.33 (broad s, 1H), 8.21-8.04 (m, | |
| | | | | 2H), 7.77 (s, 1H), 7.58-7.54 (m, | |
| | | | | 2H), 7.54 (s, 1H), 7.29 (s, 2H), | |
| | | | | 3.82-3.61 (m, 2H), 3.14-3.03 (m, | |
| | | | | 2H), 2.92 (s, 2H), 1.53 (s, 6H), | |
| | | | | 1.52 (s, 9H). | |
| 274 | 3.46 | 547.21 | A | 1H NMR (400 MHz, DMSO-d6) δ 8.22 | 22A, 13 |
| | | | | (s, 1H), 8.16 (d, J = 8.6 Hz, 2H), 7.88 | |
| | | | | (d, J = 8.8 Hz, 1H), 7.78 (s, 1H), 7.58 | |
| | | | | (d, J = 2.1 Hz, 1H), 7.56 (s, 2H), 7.20 | |
| | | | | (dd, J = 9.0, 2.9 Hz, 1H), 4.13 (t, J = | |
| | | | | 5.4 Hz, 2H), 3.78 (s, 2H), 3.61 (t, J = | |
| | | | | 5.5 Hz, 2H), 1.55 (s, 6H), 1.54 (s, 9H). | |
| 275 | 4.03 | 521.5 | A | 1H NMR (400 MHZ, DMSO-d6) δ | 1, 23 |
| | | | | 12.82 (broad s, 1H), 8.12 (d, J = 2.0 | |
| | | | | Hz, 1H), 8.05 (d, J = 1.1 Hz, 1H), 7.97 | |
| | | | | (dd, J = 8.4, 2.0 Hz, 1H), 7.79 (s, 1H), | |
| | | | | 7.75 (d, J = 1.8 Hz, 1H), 7.63 (s, 1H), | |
| | | | | 7.53 (d, J = 8.4 Hz, 1H), 4.06-3.97 | |
| | | | | (m, 2H), 3.98-3.88 (m, 2H), 2.44 (s, | |
| | | | | 3H), 1.79 (s, 6H), 1.54 (s, 9H). | |
| 276 | 4.29 | 551.56 | A | 1H NMR (400 MHz, DMSO-d6) δ 8.72- | 22B |
| | | | | 8.65 (m, 1H), 8.23-8.14 (m, 1H), 8.09- | |
| | | | | 8.01 (m, 1H), 7.85 (s, 1H), 7.77-7.68 | |
| | | | | (m, 1H), 7.66 (s, 1H), 7.33-7.27 (m, | |
| | | | | 1H), 4.20-4.13 (m, 2H), 4.08-4.02 (m, | |
| | | | | 2H), 3.31 (s, 3H), 1.83 (s, 6H), 1.53 | |
| | | | | (s, 9H). | |
| 277 | 4.15 | 537.33 | A | 1H NMR (400 MHz, DMSO-d6) δ 8.88- | 22B |
| | | | | 8.82 (m, 2H), 8.24-8.16 (m, 1H), 8.11- | |
| | | | | 8.00 (m, 1H), 7.86 (s, 1H), 7.79-7.68 | |
| | | | | (m, 1H), 7.67 (s, 1H), 7.48-7.37 (m, | |
| | | | | 1H), 4.25-4.17 (m, 2H), 4.10-4.02 (m, | |
| | | | | 2H), 1.83 (s, 6H), 1.53 (s, 9H). | |
| 278 | 3.8 | 551.52 | A | 1H NMR (400 MHz, DMSO-d6) δ | 22C, 13, 21 |
| | | | | 12.90 (broad s, 1H), 8.28-8.09 (m, | |
| | | | | 2H), 7.79 (s, 1H), 7.63 (s, 1H), 7.60- | |
| | | | | 7.46 (m, 2H), 6.88 (s, 1H), 4.19-4.07 | |
| | | | | (m, 2H), 4.06-3.90 (m, 2H), 1.80 (s, | |
| | | | | 6H), 1.53 (s, 9H). | |
| 279 | 4.21 | 561.21 | L | 1H NMR (400 MHz, DMSO-d6) δ 8.55 | 22A |
| | | | | (d, J = 2.2 Hz, 1H), 8.16 (d, J = 8.7 Hz, | |
| | | | | 2H), 7.94-7.86 (m, 1H), 7.78 (s, 1H), | |
| | | | | 7.64-7.49 (m, 3H), 4.06-3.87 (m, | |
| | | | | 2H), 3.72-3.59 (m, 2H), 3.54 (s, 2H), | |
| | | | | 2.34 (s, 3H), 1.59 (s, 6H), 1.54 (s, 9H). | |
| 280 | 3.92 | 569.53 | A | 1H NMR (400 MHz, DMSO-d6) δ | 22C, 21 |
| | | | | 13.64 (broad s, 1H), 13.49 (broad s, | |
| | | | | 1H), 8.18 (dd, J = 11.0, 2.0 Hz, 1H), | |
| | | | | 8.04 (dd, J = 8.4, 1.6 Hz, 1H), 7.85 (s, | |
| | | | | 1H), 7.71 (t, J = 8.2 Hz, 1H), 7.65 (s, | |
| | | | | 1H), 7.15 (s, 1H), 4.21-4.08 (m, 2H), | |
| | | | | 4.07-3.95 (m, 2H), 1.81 (s, 6H), 1.54 | |
| | | | | (s, 9H). | |
| 281 | 4.39 | 561.93 | A | 1H NMR (400 MHz, DMSO-d6) δ 8.97- | 22C, 13 |
| | | | | 8.92 (m, 1H), 8.31 (dd, J = 8.8, 2.3 | |
| | | | | Hz, 1H), 8.10 (d, J = 8.8 Hz, 1H), 7.53 | |
| | | | | (s, 1H), 7.18 (s, 1H), 4.32-4.22 (m, | |
| | | | | 2H), 4.09-3.98 (m, 2H), 2.79-2.63 | |
| | | | | (m, 1H), 1.90-1.63 (m, 10H), 1.47 (s, | |
| | | | | 11H), 1.41-1.27 (m, 2H), 0.97 (d, J = | |
| | | | | 9.6 Hz, 6H). | |
| 282 | 4.23 | 562.63 | A | 1H NMR (400 MHz, DMSO-d6) δ 8.96- | 22A, 13 |
| | | | | (dd, J = 2.3, 0.8 Hz, 1H), 8.32 (dd, J = | |
| | | | | 8.8, 2.4 Hz, 1H), 8.21-8.12 (m, 2H), | |
| | | | | 8.12 (dd, J = 8.8, 0.8 Hz, 1H), 7.80 (s, | |
| | | | | 1H), 7.65 (s, 1H), 7.61-7.52 (m, 2H), | |
| | | | | 4.33-4.26 (m, 2H), 4.13-4.03 (m, | |
| | | | | 2H), 1.86 (s, 6H), 1.54 (s, 9H). | |
| 283 | 3.97 | 507.82 | A | 1H NMR (400 MHz, DMSO-d6) δ | 1, 21 |
| | | | | 12.58 (s, 1H), 7.80-7.63 (m, 1H), 7.52 | |
| | | | | (s, 1H), 7.17 (s, 1H), 6.70 (t, J = 2.2 | |
| | | | | Hz, 1H), 4.15-4.06 (m, 2H), 4.01- | |
| | | | | 3.87 (m, 2H), 2.70 (tt, J = 11.8, 3.9 Hz, | |
| | | | | 1H), 1.79 (s, 6H), 1.77-1.63 (m, 4H), | |
| | | | | 1.46 (s + m, 11H), 1.32 (td, J = 13.2, | |
| | | | | 4.1 Hz, 2H), 0.97 (s, 3H), 0.94 (s, 3H). | |
| 284 | 4.08 | 539.55 | A | 1H NMR (400 MHz, DMSO-d6) δ | 1, 21 |
| | | | | 12.26 (s, 1H), 8.17 (dd, J = 11.0, 2.0 | |
| | | | | Hz, 1H), 8.03 (dd, J = 8.5, 1.7 Hz, 1H), | |
| | | | | 7.84 (s, 1H), 7.71 (t, J = 8.2 Hz, 1H), | |
| | | | | 7.64 (s, 1H), 6.47 (s, 1H), 4.15-4.04 (m, | |
| | | | | 2H), 4.03-3.89 (m, 2H), 2.22 (s, 3H), | |
| | | | | 1.79 (s, 6H), 1.53 (s, 9H). | |
| 285 | 3.97 | 520.65 | A | 1H NMR (400 MHz, DMSO-d6) δ | 1, 21 |
| | | | | 12.26 (d, J = 1.3 Hz, 1H), 8.16 (d, J = | |
| | | | | 8.6 Hz, 2H), 7.79 (s, 1H), 7.63 (s, 1H), | |
| | | | | 7.56 (d, J = 8.6 Hz, 2H), 6.47 (d, J = | |
| | | | | 1.3 Hz, 1H), 4.15-4.02 (m, 2H), 4.03- | |
| | | | | 3.89 (m, 2H), 2.22 (s, 3H), 1.79 (s, 6H), | |
| | | | | 1.53 (s, 9H). | |
| 286 | 3.96 | 565.45 | A | 1H NMR (400 MHz, DMSO-d6) δ | 22A, 11 |
| | | | | 13.41 (s, 1H), 8.22 (d, J = 5.1 Hz, 1H), | |
| | | | | 8.15 (dd, J = 11.0, 2.1 Hz, 1H), 8.02 | |
| | | | | (dd, J = 8.4, 2.1 Hz, 1H), 7.81 (s, 1H), | |
| | | | | 7.69 (t, J = 8.1 Hz, 1H), 7.54 (s, 1H), | |
| | | | | 7.11-6.91 (m, 2H), 4.13-4.03 (m, 2H), | |
| | | | | 3.93 (s, 2H), 3.73-3.54 (m, 2H), 1.66- | |
| | | | | 1.34 (m, 15H). | |
| 287 | 4.01 | 566.46 | A | 1H NMR (400 MHz, DMSO-d6) δ 8.61 | 22A, 11 |
| | | | | (dd, J = 2.3, 0.7 Hz, 1H), 8.15 (dd, J = | |
| | | | | 11.0, 2.1 Hz, 1H), 7.95 (dd, J = 9.0, 2.4 | |
| | | | | Hz, 2H), 7.81 (s, 1H), 7.69 (t, J = 8.5, | |
| | | | | 7.8 Hz, 1H), 7.53 (s, 1H), 6.65 (d, J = | |
| | | | | 9.0 Hz, 1H), 4.09 (t, J = 5.6 Hz, 2H), | |
| | | | | 3.98 (s, 2H), 3.64 (t, J = 5.7 Hz, 2H), | |
| | | | | 1.52 (s, 10H), 1.50 (s, 6H). | |
| 288 | 4.3 | 395.48 | A | 1H NMR (400 MHz, DMSO-d6) δ 8.19- | 1 |
| | | | | 8.09 (m, 2H), 7.71 (s, 1H), 7.50 (s, 1H), | |
| | | | | 7.36-7.25 (m, 2H), 3.76-3.68 (m, 2H), | |
| | | | | 3.63-3.55 (m, 2H), 1.84-1.68 (m, 2H), | |
| | | | | 1.59-1.43 (m, 6H), 1.50 (s, 9H). | |
| 289 | 3.85 | 555.44 | A | 1H NMR (400 MHz, DMSO-d6) δ 8.15 | 22A, 11 |
| | | | | (dd, J = 11.0, 2.1 Hz, 1H), 8.05-7.97 | |
| | | | | (m, 2H), 7.81 (s, 1H), 7.69 (t, J = 8.5, | |
| | | | | 7.8 Hz, 1H), 7.53 (s, 1H), 3.97 (t, J = | |
| | | | | 5.5 Hz, 2H), 3.66-3.60 (m, 4H), 1.60- | |
| | | | | 1.43 (m, 15H). | |
| 290 | 2.97 | 502.43 | A | 1H NMR (400 MHz, DMSO-d6) δ 8.13 | 14 |
| | | | | (dd, J = 11.0, 2.1 Hz, 1H), 8.00 (ddd, | |
| | | | | 1H), 7.79 (s, 1H), 7.68 (dd, J = 8.5, 7.8 | |
| | | | | Hz, 1H), 7.48 (s, 1H), 3.58-3.49 (m, | |
| | | | | 3H), 3.09 (s, 2H), 2.65 (t, J = 4.8 Hz, | |
| | | | | 2H), 2.45 (s, 1H), 1.49 (s, 9H), 1.44 (s, | |
| | | | | 6H). | |
| 291 | 3.44 | 562 | A | | 1, 11 |
| 292 | 3.22 | 510 | A | | 1 |
| 293 | 3.51 | 559 | C | | 1 |
| 294 | 3.45 | 518 | A | | 1 |
| 295 | 3.12 | 519 | A | | 1 |
| 296 | 3.42 | 522 | A | | 1 |
| 297 | 3.86 | 578 | A | | Ex. 67 |
| 298 | 3.63 | 578 | A | | Ex. 67 |
| 299 | 2.02 | 501.45 | C | 1H NMR (400 MHz, DMSO-d6) δ | 1 |
| | | | | 12.11 (s, 1H), 8.14 (dd, J = 11.0, 2.1 | |
| | | | | Hz, 1H), 8.00 (ddd, J = 8.5, 2.1, 0.7 | |
| | | | | Hz, 1H), 7.79 (s, 1H), 7.68 (dd, J = 8.5, | |
| | | | | 7.8 Hz, 1H), 7.46 (s, 1H), 3.84-3.66 | |
| | | | | (m, 1H), 3.27-3.18 (m, 1H), 2.19 (d, | |
| | | | | J = 6.9 Hz, 2H), 2.09 (s, 1H), 1.86 (d, | |
| | | | | J = 12.4 Hz, 1H), 1.61-1.54 (m, 1H), | |
| | | | | 1.51 (s, 3H), 1.50 (s, 9H), 1.45 (s, 1H), | |
| | | | | 1.41 (s, 3H), 1.35-1.26 (m, 1H). | |
| 300 | 3.37 | 615.5 | A | 1H NMR (400 MHz, CDCl3) δ 7.87 | Ex. 63 |
| | | | | (d, J = 10.0 Hz, 1H), 7.77-7.63 (m, 2H), | |
| | | | | 7.54 (t, J = 7.7 Hz, 1H), 7.49 (s, 1H), | |
| | | | | 3.98-3.84 (m, 2H), 3.81-3.43 (m, | |
| | | | | 8H), 2.96-2.83 (m, 1H), 2.45-2.30 | |
| | | | | (m, 4H), 1.62 (d, J = 4.1 Hz, 6H), 1.41 | |
| | | | | (d, J = 10.3 Hz, 3H). | |
| 301 | 1.72 | 504.53 | C | 1H NMR (400 MHz, DMSO-d6) δ | Ex. 65 |
| | | | | 12.80 (s, 1H), 8.22-8.12 (m, 2H), 8.03 | |
| | | | | (s, 1H), 7.85 (s, 1H), 7.73 (s, 1H), 7.60 | |
| | | | | (s, 1H), 7.35-7.22 (m, 2H), 3.93 (s, | |
| | | | | 4H), 2.95 (m, 1H), 2.04 (s, 2H), 1.94- | |
| | | | | 1.77 (m, 4H), 1.76 (s, 6H), 0.77 (t, J = | |
| | | | | 7.4 Hz, 6H). | |
| 302 | 1.98 | 463.47 | C | 1H NMR (400 MHz, DMSO-d6) δ 8.22- | Ex. 66 |
| | | | | 8.09 (m, 2H), 7.86 (s, 1H), 7.59 (s, | |
| | | | | 1H), 7.36-7.24 (m, 2H), 4.15-4.02 | |
| | | | | (m, 2H), 3.94 (t, J = 4.8 Hz, 2H), 2.97- | |
| | | | | 2.81 (m, 1H), 1.88-1.77 (m, 4H), 1.77 | |
| | | | | (s, 6H), 0.76 (t, J = 7.4 Hz, 6H). | |
| 303 | 2.12 | 634.5 | C | 1H NMR (400 MHz, DMSO-d6) δ | 1 |
| | | | | 12.21 (s, 1H), 7.42 (d, J = 17.3 Hz, | |
| | | | | 1H), 7.14 (d, J = 3.9 Hz, 1H), 3.95- | |
| | | | | 3.82 (m, 2H), 3.72 (t, J = 5.8 Hz, 1H), | |
| | | | | 3.62 (d, J = 26.4 Hz, 2H), 3.42 (dd, J = | |
| | | | | 7.1, 4.4 Hz, 1H), 3.04-2.92 (m, 1H), | |
| | | | | 2.67-2.50 (m, 3H), 2.45-2.30 (m, | |
| | | | | 2H), 2.15-1.96 (m, 4H), 1.91-1.76 | |
| | | | | (m, 2H), 1.63-1.52 (m, 2H), 1.46 (d, | |
| | | | | J = 19.9 Hz, 6H), 1.42 (s, 9H), 1.39- | |
| | | | | 1.32 (m, 1H), 1.26-1.11 (m, 2H), 1.06 | |
| | | | | (d, J = 2.9 Hz, 3H). | |
| 304 | 1.93 | 575.42 | C | 1H NMR (400 MHz, DMSO-d6) δ 7.42 | 1 |
| | | | | (d, J = 8.1 Hz, 1H), 7.14 (d, J = 7.4 Hz, | |
| | | | | 1H), 4.08-3.94 (m, 2H), 3.87 (s, 1H), | |
| | | | | 3.77 (s, 1H), 3.65 (dd, J = 7.0, 4.4 Hz, | |
| | | | | 1H), 3.01-2.89 (m, 1H), 2.59-2.51 | |
| | | | | (m, 2H), 2.45-2.37 (m, 2H), 2.35 (d, | |
| | | | | J = 8.7 Hz, 3H), 2.05-1.97 (m, 2H), | |
| | | | | 1.92-1.82 (m, 2H), 1.51 (s, 3H), 1.47- | |
| | | | | 1.45 (m, 1H), 1.44 (s, 9H), 1.39 (s, 3H). | |
| 305 | 1.99 | 578.5 | C | 1H NMR (400 MHz, DMSO-d6) δ 7.41 | 1 |
| | | | | (d, J = 13.4 Hz, 1H), 7.14 (d, J = 2.7 | |
| | | | | Hz, 1H), 4.97 (d, J = 18.8 Hz, 1H), | |
| | | | | 3.94-3.80 (m, 2H), 3.69-3.58 (m, 2H), | |
| | | | | 3.51 (s, 1H), 3.48-3.40 (m, 1H), 3.09- | |
| | | | | 2.95 (m, 1H), 2.88-2.74 (m, 1H), 2.62- | |
| | | | | 2.51 (m, 2H), 2.44-2.32 (m, 2H), | |
| | | | | 2.20-1.97 (m, 6H), 1.90-1.81 (m, | |
| | | | | 2H), 1.44 (d, J = 11.7 Hz, 6H), 1.42 (s, | |
| | | | | 9H), 1.24 (d, J = 11.8 Hz, 3H). | |
| 306 | 2.08 | 595.62 | C | 1H NMR (400 MHz, DMSO-d6) δ 7.38 | 1 |
| | | | | (d, J = 17.5 Hz, 1H), 7.12 (d, J = 2.4 | |
| | | | | Hz, 1H), 3.96-3.82 (m, 2H), 3.76- | |
| | | | | 3.69 (m, 1H), 3.63 (d, J = 23.2 Hz, 2H), | |
| | | | | 3.49-3.42 (m, 1H), 2.75-2.60 (m, | |
| | | | | 1H), 1.79-1.45 (m, 21H), 1.42 (s, 9H), | |
| | | | | 1.31-1.25 (m, 2H), 1.11 (s, 3H), 0.94 | |
| | | | | (s, 3H), 0.92 (s, 3H). | |
| 307 | 1.86 | 465.25 | C | 1H NMR (400 MHz, DMSO-d6) δ 7.46 | 14 |
| | | | | (s, 1H), 7.18 (s, 1H), 3.86-3.66 (m, | |
| | | | | 2H), 3.59-3.39 (m, 2H), 2.98 (t, J = | |
| | | | | 11.3 Hz, 1H), 2.17-1.77 (m, 9H), 1.66 | |
| | | | | (s, 6H), 1.43 (s, 9H). | |
| 308 | 4.72 | 480.22 | A | 1H NMR (400 MHz, DMSO-d6) δ 8.2- | Ex. 64 |
| | | | | 8.13 (m, 2H), 7.76 (s, 1H), 7.59 (s, | |
| | | | | 1H), 7.38-7.28 (m, 2H), 3.80-3.72 | |
| | | | | (m, 2H), 3.64-3.57 (m, 2H), 1.67 (s, | |
| | | | | 6H), 1.53 (s, 9H), 1.39 (s, 9H). | |
| 309 | 2.16 | 520.59 | C | 1H NMR (400 MHz, DMSO-d6) δ | Ex. 62 |
| | | | | 12.24 (d, J = 2.3 Hz, 1H), 7.49 (s, 1H), | |
| | | | | 7.15 (s, 1H), 6.44 (t, J = 1.5 Hz, 1H), | |
| | | | | 4.23-3.87 (m, 4H), 2.80-2.62 (m, | |
| | | | | 1H), 2.19 (s, 3H), 1.89-1.71 (m, 8H), | |
| | | | | 1.70-1.58 (m, 2H), 1.52-1.45 (m, | |
| | | | | 2H), 1.43 (s, 9H), 1.35-1.17 (m, 2H), | |
| | | | | 0.95 (s, 3H), 0.92 (s, 3H). | |
| 310 | 1.71 | 514.43 | C | 1H NMR (400 MHz, DMSO-d6) δ 7.93- | 1, 21 |
| | | | | 7.84 (m, 2H), 7.51 (s, 1H), 7.19 (s, | |
| | | | | 1H), 4.01-3.82 (m, 4H), 3.06-2.94 (m, | |
| | | | | 1H), 2.18-2.05 (m, 2H), 1.94-1.82 (m, | |
| | | | | 6H), 1.75 (s, 6H), 1.44 (s, 9H). | |
| 311 | 1.65 | 529.5 | C | 1H NMR (400 MHz, DMSO-d6) δ 7.40 | 3 |
| | | | | (d, J = 8.8 Hz, 1H), 7.16 (d, J = 8.3 Hz, | |
| | | | | 1H), 3.99 (t, J = 5.8 Hz, 2H), 3.93-3.84 | |
| | | | | (m, 1H), 3.80 (s, 1H), 3.68 (t, J = 5.8 | |
| | | | | Hz, 1H), 3.04-2.88 (m, 1H), 2.15-2.04 | |
| | | | | (m, 2H), 2.04-1.77 (m, 6H), 1.52 (s, 2H), | |
| | | | | 1.44 (s, 9H), 1.39 (s, 3H). | |
| 312 | 1.83 | 462.49 | C | 1H NMR (400 MHz, DMSO-d6) δ 7.46 | 14 |
| | | | | (s, 1H), 7.18 (s, 1H), 3.87-3.72 (m, | |
| | | | | 2H), 3.58-3.46 (m, 2H), 3.06-2.94 (m, | |
| | | | | 1H), 2.90 (s, 3H), 2.16-1.79 (m, 8H), | |
| | | | | 1.66 (s, 6H), 1.43 (s, 9H). | |
| 313 | 2.09 | 520.54 | C | 1H NMR (400 MHz, DMSO-d6) δ | 3, 21 |
| | | | | 12.47 (s, 1H), 7.50 (s, 1H), 7.47 (s, | |
| | | | | 1H), 7.14 (s, 1H), 4.07-3.85 (m, 4H), | |
| | | | | 2.81-2.63 (m, 1H), 1.80 (d, J = 0.8 | |
| | | | | Hz, 3H), 1.77 (s, 6H), 1.68-1.58 (m, | |
| | | | | 4H), 1.46 (s, 2H), 1.42 (s, 9H), 1.36- | |
| | | | | 1.23 (m, 2H), 0.94 (s, 3H), 0.92 (s, 3H). | |
| 314 | 1.69 | 522.69 | C | 1H NMR (400 MHz, DMSO-d6) δ 8.23- | 1 |
| | | | | 8.06 (m, 2H), 7.76 (s, 1H), 7.71 (s, | |
| | | | | 0.5H), 7.59 (s, 0.5H), 7.40-7.26 (m, | |
| | | | | 2H), 4.95 (s, 1H), 4.02-3.96 (m, 1H), | |
| | | | | 3.92 (s, 1H), 3.82-3.53 (m, 4H), 2.93- | |
| | | | | 2.67 (m, 1H), 2.28-1.91 (m, 4H), 1.52 | |
| | | | | (s, 9H), 1.45 (s, 3H), 1.37 (s, 3H), 1.25 | |
| | | | | (d, J = 6.9 Hz, 3H). Presence of | |
| | | | | rotamers, | |
| 315 | 2.03 | 506.49 | C | 1H NMR (400 MHz, DMSO-d6) δ | 1, 21 |
| | | | | 12.85 (s, 1H), 7.87 (s, 2H), 7.50 (s, | |
| | | | | 1H), 7.15 (s, 1H), 3.93 (d, J = 11.8 Hz, | |
| | | | | 4H), 2.75-2.59 (m, 1H), 1.85-1.78 | |
| | | | | (m, 2H), 1.75 (s, 6H), 1.70-1.63 (m, | |
| | | | | 2H), 1.48-1.45 (m, 2H), 1.44 (s, 9H), | |
| | | | | 1.30 (ddd, J = 15.9, 9.6, 4.1 Hz, 2H), | |
| | | | | 0.94 (s, 3H), 0.92 (s, 3H). | |
| 316 | 2.25 | 595.58 | J | 1H NMR (400 MHz, DMSO-d6) δ | 3 |
| | | | | 12.21 (brs, 1H), 7.38 (d, J = 17.0 Hz, | |
| | | | | 1H), 7.12 (d, J = 3.8 Hz, 1H), 3.92- | |
| | | | | 3.80 (m, 2H), 3.73 (d, J = 5.4 Hz, 1H), | |
| | | | | 3.62 (d, J = 26.4 Hz, 2H), 3.48-3.39 | |
| | | | | (m, 1H), 2.77-2.58 (m, 1H), 2.43 (d, J = | |
| | | | | 3.7 Hz, 1H), 2.09-1.99 (m, 2H), 1.78- | |
| | | | | 1.69 (m, 2H), 1.70-1.62 (m, 2H), | |
| | | | | 1.61-1.54 (m, 2H), 1.54 (d, J = 3.6 | |
| | | | | Hz, 3H), 1.43 (s, 6H), 1.41 (s, 9H), | |
| | | | | 1.38 (s, 1H), 1.33-1.12 (m, 5H), 1.06 | |
| | | | | (d, J = 3.1 Hz, 3H), 0.94 (s, 3H), 0.92 | |
| | | | | (d, J = 1.3 Hz, 3H). | |
| 317 | 2.2 | 454.51 | C | 1H NMR (400 MHz, DMSO-d6) δ 7.45 | 14 |
| | | | | (s, 1H), 7.14 (s, 1H), 3.81-3.69 (m, | |
| | | | | 2H), 3.52-3.45 (m, 2H), 2.90 (s, 3H), | |
| | | | | 2.75-2.60 (m, 1H), 1.82-1.70 (m, | |
| | | | | 2H), 1.66 (s, 6H), 1.65-1.58 (m, 2H), | |
| | | | | 1.48-1.43 (m, 2H), 1.42 (s, 9H), 1.30 | |
| | | | | (td, J = 13.0, 3.9 Hz, 2H), 0.94 (s, 3H), | |
| | | | | 0.92 (s, 3H). | |
| 318 | 3.59 | 525.47 | A | 1H NMR (400 MHz, DMSO-d6) δ 8.18 | 22A |
| | | | | (s, 1H), 8.14 (dd, J = 11.0, 2.1 Hz, 1H), | |
| | | | | 8.04-7.94 (m, 1H), 7.81 (s, 1H), 7.69 | |
| | | | | (t, J = 8.1 Hz, 1H), 7.56 (s, 1H), 3.79 (t, | |
| | | | | J = 5.1 Hz, 2H), 3.50 (s, 3H), 3.35 (t, | |
| | | | | J = 5.3 Hz, 2H), 3.13 (s, 2H), 1.55 (s, | |
| | | | | 6H), 1.51 (s, 9H). | |
| 319 | 4.06 | 568.8 | A | 1H NMR (400 MHz, CD3OD) δ 7.31 | 3, 11 |
| | | | | (d, J = 5.0 Hz, 1H), 7.21 (d, J = 2.9 Hz, | |
| | | | | 1H), 4.12-3.54 (m, 6H), 2.92-2.65 | |
| | | | | (m, 3H), 2.53-2.29 (m, 1H), 2.09-1.96 | |
| | | | | (m, 1H), 1.91-1.69 (m, 4H), 1.65-1.46 | |
| | | | | (m, 17H), 1.40 (m, 2H), 1.08-0.92 (m, | |
| | | | | 12H). | |
| 320 | 3.95 | 568.3 | A | 1H NMR (400 MHz, CD3OD) δ 8.09- | 3, 11 |
| | | | | 7.93 (m, 2H), 7.72 (d, J = 3.3 Hz, 1H), | |
| | | | | 7.54-7.45 (m, 2H), 7.40 (d, J = 6.2 Hz, | |
| | | | | 1H), 4.23-3.58 (m, 6H), 2.90-2.72 (m, | |
| | | | | 2H), 2.52-2.28 (m, 1H), 2.02 (m, 1H), | |
| | | | | 1.69-1.52 (m, 15H), 1.06-0.93 (m, 6H). | |
| 321 | 4.06 | 586.3 | A | 1H NMR (400 MHz, CD3OD) δ 7.93 | 3, 11 |
| | | | | (m, 1H), 7.83 (m, 1H), 7.75 (d, J = 3.4 | |
| | | | | Hz, 1H), 7.58 (m, 1H), 7.41 (d, J = 6.6 | |
| | | | | Hz, 1H), 4.21-3.87 (m, 3H), 3.86-3.74 | |
| | | | | (m, 2H), 3.68-3.59 (m, 1H), 2.98-2.69 | |
| | | | | (m, 2H), 2.53-2.27 (m, 1H), 2.02 (m, | |
| | | | | 1H), 1.67-1.56 (m, 15H), 1.09-0.87 | |
| | | | | (m, 6H). | |
| 322 | 3.36 | 456.5 | A | | 1 |
| 323 | 3.42 | 453.65 | A | | 1 |
| 324 | 4.21 | 598.1 | A | Note: There 2 protons that are not | 3 |
| | | | | accounted for and may be obscured by | |
| | | | | solvent or H2O. 1H NMR (400 MHz, | |
| | | | | DMSO-d6) δ 11.68(s, 1H), 8.10 (dt, | |
| | | | | J = 11.0, 1.7 Hz, 1H), 7.97 (dt, J = | |
| | | | | 8.4, 1.8 Hz, 1H), 7.76 (d, J = 2.6 Hz, | |
| | | | | 1H), 7.65 (td, J = 8.1, 2.1 Hz, 1H), 7.47 | |
| | | | | (d, J = 14.8 Hz, 1H), 3.89 (d, J = 5.8 | |
| | | | | Hz, 1H), 3.83 (t, J = 5.7 Hz, 1H), 3.64 | |
| | | | | (d, J = 5.6 Hz, 1H), 3.57 (s, 1H), 3.56 | |
| | | | | (s, 1H), 3.41 (t, J = 5.8 Hz, 1H), 2.67 | |
| | | | | (s, 1H), 2.61 (d, J = 4.0 Hz, 1H), 2.06- | |
| | | | | 1.90 (m, 2H), 1.53 (d, J = 6.9 Hz, 4H), | |
| | | | | 1.47 (d, J = 3.8 Hz, 12H), 1.41 (s, 3H). | |
| 325 | 4.17 | 536.44 | A | 1H NMR (400 MHz, DMSO-d6) δ 8.65 | 1 |
| | | | | (d, J = 2.3 Hz, 1H), 8.48 (dd, J = 4.7, | |
| | | | | 1.4 Hz, 1H), 8.17 (dd, J = 11.0, 2.0 Hz, | |
| | | | | 1H), 8.03 (dd, J = 8.4, 1.7 Hz, 1H), | |
| | | | | 7.88-7.82 (m, 2H), 7.71 (t, J = 8.1 Hz, | |
| | | | | 1H), 7.64 (s, 1H), 7.48 (ddd, J = 8.3, | |
| | | | | 4.8, 0.4 Hz, 1H), 4.13-4.02 (m, 2H), | |
| | | | | 4.03-3.92 (m, 2H), 1.83 (s, 6H), | |
| | | | | 1.53 (s, 9H). | |
| 326 | 4.03 | 517.67 | A | 1H NMR (400 MHz, DMSO-d6) δ 8.65 | 1 |
| | | | | (d, J = 2.3 Hz, 1H), 8.48 (dd, J = 4.7, 1.4 | |
| | | | | Hz, 1H), 8.19-8.06 (m, 2H), 7.86 (ddd, | |
| | | | | J = 8.2, 2.5, 1.5 Hz, 1H), 7.79 (s, 1H), | |
| | | | | 7.63 (s, 1H), 7.60-7.53 (m, 2H), 7.48 | |
| | | | | (ddd, J = 8.2, 4.7, 0.5 Hz, 1H), 4.14- | |
| | | | | 4.03 (m, 2H), 4.02-3.92 (m, 2H), 1.83 | |
| | | | | (s, 6H), 1.52 (s, 9H) | |
| 327 | 3.57 | 536.49 | A | 1H NMR (400 MHz, DMSO-d6) δ 8.64 | 1 |
| | | | | (apparent d, J = 6.4 Hz, 2H), 8.17 (dd, | |
| | | | | J = 11.0, 2.0 Hz, 1H), 8.04 (dd, J = 8.4, | |
| | | | | 1.8 Hz, 1H), 7.85 (s, 1H), 7.71 (t, J = | |
| | | | | 8.1 Hz, 1H), 7.67 (dd, J = 5.1, 1.4 Hz, | |
| | | | | 2H), 7.65 (s, 1H), 4.07 (s, 4H), 1.83 | |
| | | | | (s, 6H), 1.54 (s, 9H). | |
| 328 | 3.47 | 517.3 | A | 1H NMR (400 MHz, DMSO-d6) δ 8.63 | 1 |
| | | | | (d, J = 6.3 Hz, 2H), 8.20-8.02 (m, 2H), | |
| | | | | 7.79 (s, 1H), 7.69-7.60 (m, 3H), 7.59- | |
| | | | | 7.51 (m, 2H), 4.07 (s, 1H), 1.83 (s, 6H), | |
| | | | | 1.53 (s, 9H). | |
| 329 | 4.87 | 536.44 | A | 1H NMR (400 MHz, DMSO-d6) δ 8.53- | 1 |
| | | | | 8.39 (m, 1H), 8.17 (dd, J = 11.0, 2.0 Hz, | |
| | | | | 1H), 8.04 (dd, J = 8.4, 1.8 Hz, 1H), | |
| | | | | 7.95-7.78 (m, 3H), 7.71 (t, J = 8.1 Hz, | |
| | | | | 1H), 7.64 (s, 1H), 7.26 (ddd, J = 6.7, | |
| | | | | 4.9, 1.7 Hz, 1H), 4.26- 4.15 (m, 2H), | |
| | | | | 4.07-3.98 (m, 2H), 1.84 (s, 6H), 1.53 | |
| | | | | (s, 9H). | |
| 330 | 4.75 | 517.67 | A | 1H NMR (400 MHz, DMSO-d6) δ 8.54- | 1 |
| | | | | 8.38 (m, 1H), 8.20-8.04 (m, 2H), 7.94- | |
| | | | | 7.81 (m, 2H), 7.79 (s, 1H), 7.63 (s, 1H), | |
| | | | | 7.60-7.50 (m, 2H), 7.26 (ddd, J = 6.6, | |
| | | | | 4.9, 1.8 Hz, 1H), 4.28-4.14 (m, 2H), | |
| | | | | 4.10-3.94 (m, 2H), 1.84 (s, 6H), 1.53 | |
| | | | | (s, 9H). | |
| 331 | 4.12 | 590.5 | A | 1H NMR (400 MHz, DMSO-d6) δ 8.94 | 3 |
| | | | | (s, 2H), 8.15 (dd, J = 11.0, 2.1 Hz, 1H), | |
| | | | | 8.01 (dd, J = 8.5, 2.1 Hz, 1H), 7.81 (s, | |
| | | | | 1H), 7.69 (t, J = 8.1 Hz, 1H), 7.53 (s, | |
| | | | | 1H), 4.08 (d, J = 5.5 Hz, 4H), 3.80 (t, | |
| | | | | J = 5.7 Hz, 2H), 1.53 (s, 6H), 1.52 (s, | |
| | | | | 9H). | |
| 332 | 4.06 | 568.75 | A | 1H NMR (400 MHz, CD3OD) δ 7.31 | 3, 11 |
| | | | | (d, J = 5.0 Hz, 1H), 7.21 (d, J = 3.0 | |
| | | | | Hz, 1H), 4.17-3.86 (m, 3H), 3.86- | |
| | | | | 3.54 (m, 3H), 2.95-2.67 (m, 3H), | |
| | | | | 2.50-2.29 (m, 1H), 2.10-1.96 (m, | |
| | | | | 1H), 1.93-1.69 (m, 4H), 1.66-1.48 | |
| | | | | (m, 17H), 1.40 (td, J = 13.4, 4.0 Hz, | |
| | | | | 2H), 1.08-0.91 (m, 12H). | |
| 333 | 3.94 | 568.75 | A | 1H NMR (400 MHz, CD3OD) δ 8.08- | 3, 11 |
| | | | | 7.90 (m, 2H), 7.72 (d, J = 3.3 Hz, | |
| | | | | 1H), 7.49 (dd, J = 8.6, 1.8 Hz, 2H), | |
| | | | | 7.40 (d, J = 6.3 Hz, 1H), 4.17-3.89 (m, | |
| | | | | 3H), 3.86-3.58 (m, 3H), 2.97-2.73 (m, | |
| | | | | 2H), 2.55-2.25 (m, 1H), 2.01 (dt, J = | |
| | | | | 12,0, 3.9 Hz, 1H), 1.74-1.51 (m, 15H), | |
| | | | | 1.06-0.90 (m, 6H). | |
| 334 | 4.62 | 550.09 | A | 1H NMR (400 MHz, DMSO-d6) δ 9.03 | 22C |
| | | | | (s, 1H), 8.37 (s, 1H), 8.17 (dd, J = 11.0, | |
| | | | | 2.1 Hz, 1H), 8.04 (dt, J = 8.5, 1.3 Hz, | |
| | | | | 1H), 7.85 (s, 1H), 7.71 (t, J = 8.1 Hz, | |
| | | | | 1H), 7.65 (s, 1H), 4.20 (dd, J = 6.6, 3.2 | |
| | | | | Hz, 2H), 4.08 (dd, J = 6.7, 3.2 Hz, 2H), | |
| | | | | 1.86 (s, 6H), 1.54 (s, 9H). | |
| 335 | 3.5 | 474.15 | A | 1H NMR (400 MHz, DMSO-d6) δ 8.17 | 1 |
| | | | | (dd, J = 11.0, 2.1 Hz, 1H), 8.07-8.00 | |
| | | | | (m, 1H), 7.94-7.86 (m, 1H), 7.82 (s, | |
| | | | | 1H), 7.75-7.66 (m, 1H), 7.45 (s, 1H), | |
| | | | | 3.81-3.72 (m, 2H), 3.31-3.14 (m, 0H), | |
| | | | | 2.84-2.76 (m, 2H), 1.53 (s, 9H), 1.48 | |
| | | | | (s, 6H). | |
| 336 | 3.54 | 474.15 | A | 1H NMR (400 MHz, DMSO-d6) δ 8.64 | 1 |
| | | | | (d, J = 5.0 Hz, 1H), 7.50 (s, 1H), 7.26 | |
| | | | | (dd, J = 5.1, 0.6 Hz, 1H), 7.14 (s, 1H), | |
| | | | | 4.11 (dd, J = 6.0, 3.7 Hz, 2H), 3.98 (t, | |
| | | | | J = 4.7 Hz, 2H), 2.68 (tt, J = 11.7, 3.9 | |
| | | | | Hz, 1H), 2.45 (s, 3H), 1.78 (s, 6H), | |
| | | | | 1.76-1.60 (m, 4H), 1.46 (s, 2H), 1.42 | |
| | | | | (s, 9H), 1.35-1.22 (m, 2H), 0.94 (s, | |
| | | | | 3H), 0.92 (s, 3H). | |
| 337 | 3.89 | 566.75 | A | 1H NMR (400 MHz, DMSO-d6) δ 7.97 | 3, 11 |
| | | | | (dq, J = 9.1, 2.8 Hz, 2H), 7.72 (d, J = | |
| | | | | 4.7 Hz, 1H), 7.55-7.44 (m, 2H), 7.40 | |
| | | | | (d, J = 5.6 Hz, 1H), 4.04 (dt, J = 24.7, | |
| | | | | 5.9 Hz, 2H), 3.81 (t, J = 5.6 Hz, 1H), | |
| | | | | 3.73 (s, 1H), 3.70 (s, 1H), 3.61 (t, J = | |
| | | | | 5.7 Hz, 1H), 3.01 (s, 1H), 2.93 (s, 1H), | |
| | | | | 2.64-2.43 (m, 2H), 2.12-1.92 (m, J = | |
| | | | | 4.4, 3.8 Hz, 4H), 1.69-1.54 (m, 15H). | |
| 338 | 4.01 | 565.85 | A | 1H NMR (400 MHz, CD3OD) δ 7.31 | 3, 11 |
| | | | | (d, J = 4.0 Hz, 1H), 7.21 (d, J = 4.5 | |
| | | | | Hz, 1H), 4.01 (ddd, J = 24.1, 6.4, 4.4 | |
| | | | | Hz, 2H), 3.85-3.77 (m, 1H), 3.72 (s, | |
| | | | | 1H), 3.69 (s, 1H), 3.59 (t, J = 5.8 Hz, | |
| | | | | 1H), 3.00 (s, 1H), 2.92 (s, 1H), 2.72 | |
| | | | | (tq, J = 11.8, 3.7 Hz, 1H), 2.58-2.41 | |
| | | | | (m, 2H), 2.00 (qt, J = 5.9, 3.8 Hz, | |
| | | | | 4H), 1.91-1.69 (m, 4H), 1.63 (s, 3H), | |
| | | | | 1.58-1.47 (m, 14H), 1.44-1.32 (m, | |
| | | | | 2H), 1.03 (d, J = 1.8 Hz, 3H), 0.97 | |
| | | | | (d, J = 1.7 Hz, 3H). | |
| 339 | 4.51 | 532.75 | A | 1H NMR (400 MHz, DMSO-d6) δ 8.64 | 22A |
| | | | | (d, J = 5.0 Hz, 1H). 7.50 (s, 1H), 7.26 | |
| | | | | (dd, J = 5.1, 0.6 Hz, 1H), 7.14 (s, 1H), | |
| | | | | 4.11 (dd, J = 6.0, 3.7 Hz, 2H), 3.98 (t, | |
| | | | | J = 4.7 Hz, 2H), 2.68 (tt, J = 11.7, 3.9 | |
| | | | | Hz, 1H), 2.45 (s, 3H), 1.78 (s, 6H), | |
| | | | | 1.76-1.60 (m, 4H), 1.46 (s, 2H), 1.42 | |
| | | | | (s, 9H), 1.35-1.22 (m, 2H), 0.94 (s, | |
| | | | | 3H), 0.92 (s, 3H). | |
| 340 | 4.3 | 532.38 | A | 1H NMR (400 MHz, DMSO-d6) δ 8.65 | 22A |
| | | | | (d, J = 5.1 Hz, 1H), 8.13 (d, J = 8.6 Hz, | |
| | | | | 2H), 7.76 (s, 1H), 7.62 (s, 1H), 7.53 (d, | |
| | | | | J = 8.6 Hz, 2H), 7.26 (dd, J = 5.1, 0.6 | |
| | | | | Hz, 1H), 4.13 (t, J = 4.7 Hz, 2H), 4.02 | |
| | | | | (d, J = 5.3 Hz, 2H), 2.46 (s, 3H), 1.80 | |
| | | | | (s, 6H), 1.50 (s, 9H). | |
| 341 | 4.03 | 572.1 | A | 1H NMR (400 MHz, DMSO-d6) δ 8.94 | 3 |
| | | | | (s, 2H), 8.13 (d, J = 8.5 Hz, 2H), 7.76 | |
| | | | | (s, 1H), 7.56-7.49 (m, 3H), 4.08 (d, J = | |
| | | | | 6.3 Hz, 4H), 3.80 (t, J = 5.7 Hz, 2H), | |
| | | | | 1.53 (s, 6H), 1.52 (s, 9H). | |
| 342 | 4.34 | 602.01 | A | 1H NMR (400 MHz, DMSO-d6) δ 8.18 | Ex. 61 |
| | | | | (dd, J = 11.0, 2.1 Hz, 1H), 8.05 (ddd, | |
| | | | | J = 8.5, 2.2, 0.7 Hz, 1H), 7.96 (d, J = | |
| | | | | 2.4 Hz, 1H), 7.86 (s, 1H), 7.73 (dd, J = | |
| | | | | 8.4, 7.8 Hz, 1H), 7.67 (s, 1H), 6.93 (d, | |
| | | | | J = 2.4 Hz, 1H), 4.14 (dd, J = 7.1, 2.7 | |
| | | | | Hz, 2H), 4.05-3.99 (m, 2H), 3.95 (s, | |
| | | | | 3H), 1.69 (s, 6H), 1.55 (s, 9H). | |
| 343 | 4.04 | 586.85 | A | 1H NMR (400 MHz, CD3OD) δ 7.91 | 1, 11 |
| | | | | (m, 1H), 7.81 (m, 1H), 7.74 (d, J = 3.5 | |
| | | | | Hz, 1H), 7.56 m, 1H), 7.40 (d, J = 6.8 | |
| | | | | Hz, 1H), 4.14-3.88 (m, 3H), 3.87- | |
| | | | | 3.73 (m, 2H), 3.70-3.52 (m, 1H), | |
| | | | | 2.99-2.72 (m, 2H), 2.53-2.28 (m, 1H), | |
| | | | | 2.01 (m, 1H), 1.69-1.49 (m, 15H), | |
| | | | | 1.06-0.93 (m, 6H). | |
| 344 | 4.42 | 589.45 | A | 1H NMR (400 MHz, DMSO-d6) δ 8.15 | 3 |
| | | | | (dd, J = 11.0, 2.1 Hz, 1H), 8.03-7.99 | |
| | | | | (m, 1H), 7.81 (s, 1H), 7.72-7.65 (m, | |
| | | | | 2H), 7.53 (s, 1H), 7.36 (d, J = 7.2 Hz, | |
| | | | | 1H), 6.66 (d, J = 8.6 Hz, 1H), 4.14- | |
| | | | | 4.01 (m, 4H), 3.62 (t, J = 5.6 Hz, 2H), | |
| | | | | 1.60-1.45 (m, 15H). | |
| 345 | 4.51 | 570.8 | A | 1H NMR (400 MHz, CD3OD) δ 7.69 | 3 |
| | | | | (dd, J = 8.6, 7.3 Hz, 1H), 7.44 (dd, J = | |
| | | | | 7.4, 0.6 Hz, 1H), 7.32 (s, 1H), 7.22 (s, | |
| | | | | 1H), 6.71 (d, J = 8.6 Hz, 1H), 4.16 (d, | |
| | | | | J = 4.9 Hz, 4H), 3.77-3.70 (m, 2H), | |
| | | | | 2.74 (tt, J = 12.0, 3.8 Hz, 1H), 1.91- | |
| | | | | 1.70 (m, 6H), 1.63 (s, 6H), 1.53 (s, 9H), | |
| | | | | 1.42 (dd, J = 13.3, 4.0 Hz, 2H), 1.04 (s, | |
| | | | | 3H), 0.97 (s, 3H). | |
| 346 | 4.17 | 571.7 | A | 1H NMR (400 MHz, CD3OD) δ 8.95 | 3 |
| | | | | (s, 2H), 7.33 (s, 1H), 7.22 (s, 1H), 4.18- | |
| | | | | 4.07 (m, 4H), 3.90 (t, J = 5.7 Hz, 2H), | |
| | | | | 2.74 (tt, J = 12.1, 3.8 Hz, 1H), 1.90- | |
| | | | | 1.69 (m, 6H), 1.63 (s, 6H), 1.54 (s, | |
| | | | | 9H), 1.46-1.36 (m, 2H), 1.04 (s, 3H), | |
| | | | | 0.97 (s, 3H). | |
| 347 | 3.12 | 555.72 | A | 1H NMR (400 MHz, CD3OD) δ 8.00 | 1 |
| | | | | (d, J = 8.3 Hz, 2H), 7.75 (d, J = 1.4 Hz, | |
| | | | | 1H), 7.52 (d, J = 8.4 Hz, 2H), 7.43 (s, | |
| | | | | 1H), 4.20-4.15 (m, 2H), 4.15-4.04 | |
| | | | | (m, 2H), 3.82 (s, 1H), 3.75 (dd, J = | |
| | | | | 11.6, 6.2 Hz, 2H), 3.70 (s, 1H), 3.64 (d, | |
| | | | | J = 8.4 Hz, 2H), 2.90 (d, J = 11.4 Hz, | |
| | | | | 3H), 1.66 (s, 3H), 1.64 (s, 3H), 1.60 | |
| | | | | (2s, 9H). Some splitting in signal due | |
| | | | | to rotamers, | |
| 348 | 3.13 | 522.58 | A | 1H NMR (400 MHz, DMSO-d6) δ 8.89 | 1 |
| | | | | (d, J = 3.0 Hz, 1H), 8.61 (d, J = 6.3 Hz, | |
| | | | | 1H), 8.17 (dd, J = 11.0, 2.0 Hz, 1H), | |
| | | | | 8.03 (dd, J = 8.5, 1.8 Hz, 1H), 7.83 (s, | |
| | | | | 1H), 7.71 (t, J = 8.1 Hz, 1H), 7.55 (s, | |
| | | | | 1H), 6.80 (dd, J = 6.3, 3.3 Hz, 1H), | |
| | | | | 4.12 (t, J = 5.3 Hz, 2H), 3.78 (s, 2H), | |
| | | | | 3.56 (t, J = 5.4 Hz, 2H), 1.54 (s, 9H), | |
| | | | | 1.53 (s, 6H). | |
| 349 | 2.98 | 504.51 | A | 1H NMR (400 MHz, DMSO-d6) δ 8.89 | 1 |
| | | | | (d, J = 3.0 Hz, 1H), 8.61 (d, J = 6.3 Hz, | |
| | | | | 1H), 8.15 (d, J = 8.6 Hz, 2H), 7.77 (s, | |
| | | | | 1H), 7.58-7.53 (m, 3H), 6.80 (dd, J = | |
| | | | | 6.3, 3.2 Hz, 1H), 4.12 (t, J = 5.5 Hz, | |
| | | | | 2H), 3.78 (s, 2H), 3.56 (t, J = 5.5 Hz, | |
| | | | | 2H), 1.53 (s, 15H). | |
| 350 | 4.3 | 571.5 | A | 1H NMR (400 MHz, DMSO-d6) δ 8.13 | 3 |
| | | | | (d, J = 8.6 Hz, 2H), 7.75 (s, 1H), 7.69 | |
| | | | | (dd, J = 8.6, 7.3 Hz, 1H), 7.56-7.51 | |
| | | | | (m, 3H), 7.37 (d, J = 7.2 Hz, 1H), | |
| | | | | 6.69 (d, J = 8.6 Hz, 1H), 4.08 (d, J = | |
| | | | | 5.5 Hz, 4H), 3.63 (t, J = 5.7 Hz, 2H), | |
| | | | | 1.52 (m, 15H). | |
| 351 | 4.13 | 565 | A | 1H NMR (400 MHz, DMSO-d6) δ | 22A, 11 |
| | | | | 12.95 (s, 1H), 8.26 (dd, J = 4.7, 1.9 Hz, | |
| | | | | 1H), 8.17 (dd, J = 11.0, 2.1 Hz, 1H), | |
| | | | | 8.04 (dd, J = 8.5, 2.0 Hz, 1H), 7.94 | |
| | | | | (dd, J = 7.5, 1.9 Hz, 1H), 7.83 (s, 1H), | |
| | | | | 7.71 (t, J = 8.1 Hz, 1H), 7.55 (s, 1H), | |
| | | | | 6.78 (dd, J = 7.6, 4.7 Hz, 1H), 4.04 (t, | |
| | | | | J = 5.4 Hz, 2H), 3.65 (s, 2H), 3.58- | |
| | | | | 3.47 (m, 2H), 1.54 (s, 6H), 1.53 | |
| | | | | (s, 9H). | |
| 352 | 4.53 | 579.15 | A | 1H NMR (400 MHz, DMSO-d6) δ | 22A |
| | | | | 12.72 (s, 1H), 8.17 (dd, J = 11.0, 2.1 | |
| | | | | Hz, 1H), 8.03 (dd, J = 8.5, 2.0 Hz, 1H), | |
| | | | | 7.85 (d, J = 7.7 Hz, 2H), 7.83 (s, 1H), | |
| | | | | 7.71 (t, J = 8.1 Hz, 1H), 7.54 (s, 1H), | |
| | | | | 6.64 (d, J = 7.7 Hz, 1H), 4.02 (t, J = 5.3 | |
| | | | | Hz, 2H), 3.64 (s, 2H), 3.51 (t, J = 5.5 | |
| | | | | Hz, 2H), 2.37 (s, 3H), 1.54 (s, 14H), | |
| | | | | 1.53 (s, 14H). | |
| 353 | 4.45 | 536.63 | A | 1H NMR (400 MHz, DMSO-d6) δ 8.14 | 12 |
| | | | | (dd, J = 11.0, 2.1 Hz, 1H), 8.01 (dd, J = | |
| | | | | 8.5, 2.0 Hz, 1H), 7.82 (s, 1H), 7.69 (t, | |
| | | | | J = 8.2 Hz, 1H), 7.61 (s, 1H), 3.95 (s, | |
| | | | | 4H), 3.44 (s, 3H), 1.75 (s, 6H), 1.51 (s, | |
| | | | | 9H). | |
| 354 | 4.19 | 568.55 | A | 1H NMR (400 MHz, DMSO-d6) δ | 3 |
| | | | | 12.20 (s, 1H), 7.43, 7.41 (s, 1H), 7.16 | |
| | | | | (s, 1H), 3.91 (dt, J = 19.9, 5.7 Hz, 2H), | |
| | | | | 3.68 (t, J = 5.5 Hz, 1H), 3.63 (d, J = 3.4 | |
| | | | | Hz, 2H), 3.49 (t, J = 5.8 Hz, 1H), 2.78- | |
| | | | | 2.64 (m, 1H), 2.32-2.19 (m, 2H), 1.85- | |
| | | | | 1.64 (m, 6H), 1.52 (s, 3H), 1.47 (s, | |
| | | | | 3H), 1.46, 1.46 (s, 9H), 1.34 (td, J = | |
| | | | | 13.3, 4.3 Hz, 2H), 1.12 (s, 3H), 1.11 (s, | |
| | | | | 3H), 0.98 (s, 3H), 0.96 (s, 3H). | |
| 355 | 4.26 | 550.09 | A | 1H NMR (400 MHz, DMSO-d6) δ 8.15 | 22C |
| | | | | (dd, J = 11.0, 2.1 Hz, 1H), 8.04-8.00 | |
| | | | | (m, 1H), 7.98 (d, J = 9.1 Hz, 1H), 7.83 | |
| | | | | (s, 1H), 7.69 (t, J = 8.1 Hz, 1H), 7.63 | |
| | | | | (s, 1H), 7.60 (d, J = 9.1 Hz, 1H), 4.40- | |
| | | | | 4.25 (m, 2H), 4.06 (t, J = 4.9 Hz, 2H), | |
| | | | | 2.59 (s, 3H), 1.83 (s, 6H), 1.51 (s, 9H). | |
| 356 | 4.24 | 539.09 | A | 1H NMR (400 MHz, DMSO-d6) δ 8.36 | 22C |
| | | | | (s, 1H), 8.15 (dd, J = 11.0, 2.1 Hz, 1H), | |
| | | | | 8.05-7.97 (m, 1H), 7.83 (s, 1H), 7.69 | |
| | | | | (t, J = 8.1 Hz, 1H), 7.64 (s, 1H), 4.22 | |
| | | | | (dd, J = 6.2, 3.5 Hz, 2H), 4.04 (s, 5H), | |
| | | | | 1.79 (s, 6H), 1.52 (s, 9H). | |
| 357 | 4.2 | 536.09 | A | 1H NMR (400 MHz, DMSO-d6) δ 9.07 | 22C |
| | | | | (dd, J = 4.7, 1.4 Hz, 1H), 8.19-8.09 | |
| | | | | (m, 2H), 8.04-7.98 (m, 1H), 7.83 (s, | |
| | | | | 1H), 7.73 (dd, J = 9.0, 4.7 Hz, 1H), | |
| | | | | 7.69 (t, J = 8.2 Hz, 1H), 7.64 (s, 1H), | |
| | | | | 4.34 (dd, J = 6.5, 3.3 Hz, 2H), 4.08 | |
| | | | | (dd, J = 6.5, 3.3 Hz, 2H), 1.84 (s, 6H), | |
| | | | | 1.52 (s, 9H). | |
| 358 | 4.7 | 539.47 | A | 1H NMR (400 MHz, DMSO-d6) δ 8.18 | 22C |
| | | | | (dd, J = 11.0, 2.1 Hz, 1H), 8.08-7.97 | |
| | | | | (m, 2H), 7.86 (s, 1H), 7.76-7.70 (m, | |
| | | | | 1H), 7.65 (s, 1H), 4.12 (s, 5H), 4.08- | |
| | | | | 3.99 (m, 2H), 1.83 (s, 6H), 1.55 (s, 9H). | |
| 359 | 4.46 | 550.44 | A | 1H NMR (400 MHz, DMSO-d6) δ 8.52 | 22C |
| | | | | (d, J = 2.5 Hz, 1H), 8.44 (dd, J = 2.5, | |
| | | | | 0.8 Hz, 1H), 8.15 (dd, J = 11.0, 2.1 Hz, | |
| | | | | 1H), 8.06-7.92 (m, 1H), 7.82 (s, 1H), | |
| | | | | 7.69 (t, J = 8.1 Hz, 1H), 7.64 (s, 1H), | |
| | | | | 4.07 (s, 4H), 2.39 (s, 3H), 1.83 (s, 6H), | |
| | | | | 1.50 (s, 9H). | |
| 360 | 4.63 | 536.3 | A | 1H NMR (400 MHz, DMSO-d6) δ 9.24 | 22C |
| | | | | (d, J = 1.5 Hz, 1H), 8.57 (dd, J = 2.6, | |
| | | | | 1.5 Hz, 1H), 8.48 (d, J = 2.5 Hz, 1H), | |
| | | | | 8.18 (dd, J = 11.0, 2.1 Hz, 1H), 8.04 | |
| | | | | (ddd, J = 8.5, 2.1, 0.7 Hz, 1H), 7.86 (s, | |
| | | | | 1H), 7.72 (dd, J = 8.5, 7.8 Hz, 1H), | |
| | | | | 7.66 (s, 1H), 4.22 (dd, J = 6.3, 3.4 Hz, | |
| | | | | 2H), 4.09 (dd, J = 6.6, 3.2 Hz, 2H), | |
| | | | | 1.87 (s, 6H), 1.54 (s, 9H). | |
| 361 | 4.08 | 519.24 | A | 1H NMR (400 MHz, DMSO-d6) δ 8.84 | 22A |
| | | | | (d, J = 4.8 Hz, 2H), 7.54 (S, 1H), 7.40 | |
| | | | | (t, J = 4.8 Hz, 1H), 7.18 (s, 1H), 4.18 | |
| | | | | (dd, J = 6.0, 3.8 Hz, 2H), 4.02 (t, J = | |
| | | | | 4.9 Hz, 2H), 2.71 (ddt, J = 11.6, 7.8, | |
| | | | | 3.9 Hz, 1H), 1.82 (s, 6H), 1.80-1.61 | |
| | | | | (m, 4H), 1.49 (s, 1H), 1.45 (s, 9H), | |
| | | | | 1.33 (td, J = 13.3, 4.2 Hz, 2H), 0.97 (s, | |
| | | | | 3H), 0.95 (s, 3H). | |
| 362 | 3.91 | 610.3 | A | 1H NMR (400 MHz, CD3OD) δ 7.92 | 3 |
| | | | | (dt, J = 10.7, 2.0 Hz, 1H), 7.82 (dtd, | |
| | | | | J = 8.5, 2.1, 0.8 Hz, 1H), 7.75 (d, J = | |
| | | | | 3.6 Hz, 1H), 7.58 (ddd, J = 8.4, 7.6, | |
| | | | | 1.7 Hz, 1H), 7.40 (d, J = 3.1 Hz, 1H), | |
| | | | | 4.02 (dd, J = 7.0, 4.5 Hz, 2H), 3.78- | |
| | | | | 3.48 (m, 4H), 3.25 (d, J = 8.5 Hz, | |
| | | | | 1H), 2.97 (tt, J = 8.4, 4.2 Hz, 1H), | |
| | | | | 2.41-2.28 (m, 4H), 2.24-2.11 (m, | |
| | | | | 4H), 1.61-1.53 (m, 15H). | |
| 363 | 4.17 | 551.15 | A | 1H NMR (400 MHz, DMSO-d6) δ 8.15 | 3 |
| | | | | (dd, J = 11.0, 2.1 Hz, 1H), 8.01 (dd, | |
| | | | | J = 8.5, 2.0 Hz, 1H), 7.86 (s, 1H), | |
| | | | | 7.81 (s, 1H), 7.69 (t, J = 8.1 Hz, 1H), | |
| | | | | 7.56 (s, 1H), 7.48-6.00 (bs, 2H), 3.73- | |
| | | | | 3.63 (m, 2H), 2.86 (t, J = 5.2 Hz, 2H), | |
| | | | | 1.80 (s, 6H), 1.52 (s, 9H). | |
| 364 | 3.89 | 592.4 | A | 1H NMR (400 MHz, CD3OD) δ 7.30 | 3 |
| | | | | (d, J = 1.5 Hz, 1H), 7.21 (d, J = 3.1 | |
| | | | | Hz, 1H), 4.05-3.93 (m, 2H), 3.76-3.54 | |
| | | | | (m, 4H), 3.24 (q, J = 8.5 Hz, 1H), 2.98 | |
| | | | | (pd, J = 8.4, 4.6 Hz, 1H), 2.81-2.67 (m, | |
| | | | | 1H), 2.47-2.28 (m, 4H), 2.24-2.12 (m, | |
| | | | | 4H), 1.90-1.68 (m, 4H), 1.62-1.49 (m, | |
| | | | | 17H), 1.40 (td, J = 13.5, 4.1 Hz, 2H), | |
| | | | | 1.03 (d, J = 1.1 Hz, 3H), 0.97 (d, J = | |
| | | | | 1.1 Hz, 3H). | |
| 365 | 4.62 | 599.15 | A | 1H NMR (400 MHz, DMSO-d6) δ 8.60 | 22A |
| | | | | (d, J = 1.9 Hz, 1H), 8.14 (dd, J = 11.0, | |
| | | | | 2.1 Hz, 1H), 8.05-7.95 (m, 2H), 7.80 | |
| | | | | (s, 1H), 7.68 (t, J = 8.1 Hz, 1H), 7.53 | |
| | | | | (s, 1H), 3.97 (t, J = 5.4 Hz, 2H), 3.82 | |
| | | | | (dd, J = 6.8, 4.0 Hz, 2H), 3.75 (s, 2H), | |
| | | | | 1.55 (s, 6H), 1.51 (s, 9H). | |
| 366 | 3.88 | 568.15 | A | 1H NMR (400 MHz, DMSO-d6) δ | 3 |
| | | | | 12.22 (s, 1H), 8.19-8.13 (m, 2H), 7.78 | |
| | | | | (d, J = 1.1 Hz, 1H), 7.60-7.55 (m, 2H), | |
| | | | | 7.54, 7.52 (s, 1H), 3.97 (t, J = 5.5 Hz, | |
| | | | | 1H), 3.92 (t, J = 5.8 Hz, 1H), 3.70 (t, | |
| | | | | J = 5.5 Hz, 1H), 3.65 (d, J = 3.5 Hz, | |
| | | | | 2H), 3.51 (dd, J = 6.9, 4.6 Hz, 1H), | |
| | | | | 2.33-2.20 (m, 2H), 1.73 (dq, J = 12.6, | |
| | | | | 7.4, 5.9 Hz, 2H), 1.54, 1.53 (s, J = | |
| | | | | 2.0 Hz, 12H, Me obscured by tBu), | |
| | | | | 1.49 (s, 3H), 1.13 (s, 3H), 1.11 (s, 3H). | |
| 367 | 4 | 586.15 | A | 1H NMR (400 MHz, DMSO-d6) δ | 3 |
| | | | | 12.17 (s, 1H), 8.17 (dd, J = 11.0, 2.0 | |
| | | | | Hz, 1H), 8.03 (dd, J = 8.5, 2.0 Hz, 1H), | |
| | | | | 7.83, 7.83 (s, 1H), 7.75-7.66 (m, 1H), | |
| | | | | 7.54, 7.52 (s, 1H), 3.96 (t, J = 5.5 Hz, | |
| | | | | 1H), 3.91 (dd, J = 7.2, 4.5 Hz, 1H), | |
| | | | | 3.69 (t, J = 5.6 Hz, 1H), 3.64 (d, J = | |
| | | | | 4.2 Hz, 2H), 3.50 (t, J = 5.7 Hz, 1H), | |
| | | | | 2.34-2.17 (m, 2H), 1.73 (dt, J = 12.2, | |
| | | | | 4.5 Hz, 2H), 1.53, 1.53 (s, 12H, methyl | |
| | | | | obscured by this peak), 1.48 (s, 3H), | |
| | | | | 1.12 (s, 3H), 1.10 (s, 3H). | |
| 368 | 4.17 | 505.4 | A | 1H NMR (400 MHz, DMSO-d6) δ 8.49 | 3 |
| | | | | (s, 1H), 8.36 (s, 2H), 8.18-8.09 (m, 2H), | |
| | | | | 7.77 (s, 1H), 7.60-7.51 (m, 3H), 4.08 (t, | |
| | | | | J = 5.5 Hz, 2H), 3.68 (s, 2H), 3.54 (t, | |
| | | | | J = 5.5 Hz, 2H), 1.54 (s, 6H), 1.53 | |
| | | | | (s, 9H). | |
| 369 | 3.97 | 563.28 | A | 1H NMR (400 MHz, DMSO-d6) δ 9.14 | 22B |
| | | | | (s, 2H), 8.23-8.02 (m, 2H), 7.79 (s, | |
| | | | | 1H), 7.65 (s, 1H), 7.60-7.47 (m, 2H), | |
| | | | | 4.31-4.20 (m, 2H), 4.14-3.98 (m, 2H), | |
| | | | | 1.83 (s, 5H), 1.52 (s, 9H). | |
| 370 | 4.89 | 522.09 | A | 1H NMR (400 MHz, DMSO-d6) δ 8.49 | 22A |
| | | | | (d, J = 0.9 Hz, 2H), 8.16 (d, J = 8.7 Hz, | |
| | | | | 2H), 7.78 (s, 1H), 7.56 (d, J = 8.7 Hz, | |
| | | | | 2H), 7.53 (s, 1H), 4.06 (t, J = 5.8 Hz, | |
| | | | | 2H), 3.97 (s, 2H), 3.71 (t, J = 5.7 Hz, | |
| | | | | 2H), 1.54 (s, 9H), 1.53 (s, 6H). | |
| 371 | 4.52 | 552.44 | A | 1H NMR (400 MHz, DMSO-d6) δ 9.06 | 22C |
| | | | | (d, J = 1.5 Hz, 1H), 8.44 (dd, J = 1.5, | |
| | | | | 0.7 Hz, 1H), 8.18 (dd, J = 11.0, 2.1 Hz, | |
| | | | | 1H), 8.04 (dt, J = 8.4, 1.3 Hz, 1H), 7.85 | |
| | | | | (s, 1H), 7.72 (t, J = 8.1 Hz, 1H), 7.65 | |
| | | | | (s, 1H), 4.17 (dd, J = 6.5, 3.2 Hz, 2H), | |
| | | | | 4.07 (d, J = 5.4 Hz, 2H), 2.50 (s, 3H), | |
| | | | | 1.86 (s, 6H), 1.54 (s, 9H). | |
| 372 | | | | 1H NMR (400 MHz, CD3OD) δ 7.92 | 3 |
| | | | | (ddd, J = 10.7, 2.9, 2.0 Hz, 1H), 7.87- | |
| | | | | 7.79 (m, 1H), 7.74 (d, J = 4.9 Hz, 1H), | |
| | | | | 7.58 (ddd, J = 8.4, 7.6, 2.6 Hz, 1H), | |
| | | | | 7.41 (d, J = 6.1 Hz, 1H), 4.25-3.97 | |
| | | | | (m, 2H), 3.81 (dd, J = 6.3, 4.9 Hz, 1H), | |
| | | | | 3.72 (d, J = 11.2 Hz, 2H), 3.65-3.56 | |
| | | | | (m, 1H), 3.01 (s, 1H), 2.93 (s, 1H), | |
| | | | | 2.59-2.47 (m, 2H), 2.15-1.95 (m, | |
| | | | | 4H), 1.66-1.53 (m, 15H). | |
| 373 | 4.08 | 586.25 | A | 1H NMR (400 MHz, CD3OD) δ 7.93 | 3 |
| | | | | (ddd, J = 10.7, 2.1, 1.0 Hz, 1H), 7.83 | |
| | | | | (ddt, J = 8.4, 2.0, 1.0 Hz, 1H), 7.75 (d, | |
| | | | | J = 2.1 Hz, 1H), 7.58 (ddd, J = 8.8, 7.6, | |
| | | | | 1.3 Hz, 1H), 7.41 (d, J = 2.6 Hz, 1H), | |
| | | | | 4.20 (dt, J = 11.7, 5.9 Hz, 1H), 4.06 (t, | |
| | | | | J = 5.7 Hz, 2H), 3.92-3.80 (m, 2H), | |
| | | | | 3.73-3.59 (m, 1H), 3.19-2.87 (m, | |
| | | | | 1H), 2.72 (dd, J = 17.0, 11.2 Hz, 1H), | |
| | | | | 2.53 (td, J = 17.0, 3.5 Hz, 1H), 1.93- | |
| | | | | 1.77 (m, 1H), 1.69-1.54 (m, 15H), | |
| | | | | 1.03-0.93 (m, 6H). | |
| 374 | 4.08 | 586.25 | A | 1H NMR (400 MHz, CD3OD) δ 7.93 | 3 |
| | | | | (ddd, J = 10.7, 2.1, 1.1 Hz, 1H), 7.83 | |
| | | | | (ddt, J = 8.4, 1.9, 1.0 Hz, 1H), 7.75 | |
| | | | | (d, J = 2.1 Hz, 1H), 7.58 (ddd, J = | |
| | | | | 8.8, 7.6, 1.3 Hz, 1H), 7.41 (d, J = | |
| | | | | 2.5 Hz, 1H), 4.20 (dt, J = 11.6, 5.9 | |
| | | | | Hz, 1H), 4.06 (dd, J = 7.3, 4.1 Hz, | |
| | | | | 2H), 3.93-3.78 (m, 2H), 3.77-3.55 | |
| | | | | (m, 1H), 3.16-2.86 (m, 1H), 2.79- | |
| | | | | 2.67 (m, 1H), 2.53 (td, J = 17.1, 3.5 | |
| | | | | Hz, 1H), 1.86 (dp, J = 20.6, 6.8 Hz, | |
| | | | | 1H), 1.67 (d, J = 4.5 Hz, 2H), 1.62- | |
| | | | | 1.54 (m, 13H), 1.09-0.91 (m, 6H). | |
| 375 | 3.9 | 598.25 | A | 1H NMR (400 MHz, CD3OD) δ 7.93 | 3 |
| | | | | (dt, J = 10.7, 1.8 Hz, 1H), 7.83 (dt, | |
| | | | | J = 8.6, 1.9 Hz, 1H), 7.75 (d, J = | |
| | | | | 2.9 Hz, 1H), 7.63-7.53 (m, 1H), 7.41 | |
| | | | | (d, J = 4.7 Hz, 1H), 4.05 (td, J = 7.7, | |
| | | | | 4.5 Hz, 2H), 3.97-3.83 (m, 1H), 3.78 | |
| | | | | (d, J = 12.4 Hz, 2H), 3.69-3.57 (m, | |
| | | | | 1H), 2.87-2.62 (m, 1H), 2.55-2.28 | |
| | | | | (m, 1H), 2.00 (d, J = 12.4 Hz, 2H), | |
| | | | | 1.94-1.86 (m, 1H), 1.76 (d, J = 17.9 | |
| | | | | Hz, 1H), 1.65-1.57 (m, 15H), 1.50- | |
| | | | | 1.23 (m, 4H). | |
| 376 | 3.2 | 578.2 | A | 1H NMR (400 MHz, DMSO-d6) δ 7.11 | 3 |
| | | | | (ddd, J = 10.7, 2.1, 1.2 Hz, 1H), 7.01 | |
| | | | | (ddt, J = 8.4, 2.0, 1.0 Hz, 1H), 6.93 (d, | |
| | | | | J = 1.9 Hz, 1H), 6.83-6.69 (m, 1H), | |
| | | | | 6.60 (d, J = 3.2 Hz, 1H), 3.26 (m, 2H), | |
| | | | | 3.09-2.91 (m, 3H), 2.87 (t, J = 5.8 Hz, | |
| | | | | 1H), 1.89 (m, 2H), 1.32-1.20 (m, 2H), | |
| | | | | 0.84-0.75 (m, 15H), 0.66 (m, 3H). | |
| 377 | 2.77 | 555.35 | A | 1H NMR (400 MHz, DMSO-d6) δ 6.50 | 3 |
| | | | | (d, J = 0.6 Hz, 1H), 6.40 (d, J = 1.0 Hz, | |
| | | | | 1H), 3.37-3.07 (m, 2H), 3.02-2.72 (m, | |
| | | | | 5H), 2.02-1.71 (m, 3H), 1.45-1.25 (m, | |
| | | | | 2H), 1.12-0.88 (m, 4H), 0.82-0.70 (m, | |
| | | | | 17H), 0.59 (m, 2H), 0.22 (s, 3H), 0.16 | |
| | | | | (s, 3H). | |
| 378 | 3.6 | 540.35 | A | 1H NMR (400 MHz, CD3OD) δ 7.35 (s, | 3 |
| | | | | 1H), 7.25 (s, 1H), 4.05 (dt, J = 13.9, | |
| | | | | 5.8 Hz, 2H), 3.87-3.75 (m, 3H), 3.68 (t, | |
| | | | | J = 5.8 Hz, 1H), 2.77 (tt, J = 12.0, 3.9 | |
| | | | | Hz, 1H), 2.57-2.36 (m, 4H), 1.99-1.72 | |
| | | | | (m, 6H), 1.69-1.52 (m, 17H), 1.44 (td, | |
| | | | | J = 13.4, 4.1 Hz, 2H), 1.07 (s, 3H), 1.01 | |
| | | | | (s, 3H). | |
| 379 | 3.59 | 575.7 | A | 1H NMR (400 MHz, CD3OD) δ 9.20 | 3 |
| | | | | (dd, J = 9.9, 2.1 Hz, 1H), 8.53 (td, J = | |
| | | | | 7.7, 2.1 Hz, 1H), 7.83 (d, J = 8.0 Hz, | |
| | | | | 1H), 7.36 (d, J = 14.2 Hz, 1H), 7.25 | |
| | | | | (d, J = 19.1 Hz, 1H), 4.17 (dd, J = | |
| | | | | 7.1, 4.5 Hz, 1H), 4.04-3.75 (m, 5H), | |
| | | | | 2.86-2.67 (m, 1H), 1.93-1.69 (m, | |
| | | | | 6H), 1.57 (d, J = 4.4 Hz, 11H), 1.46 | |
| | | | | (s, 6H), 1.04 (dd, J = 26.0, 7.1 Hz, 6H). | |
| 380 | 3.57 | 575.35 | A | 1H NMR (400 MHz, CD3OD) δ 8.80 | 3 |
| | | | | (s, (s, 1H), 8.27 (t, J = 8.9 Hz, 1H), 8.12 | |
| | | | | (d, J = 5.3 Hz, 1H), 7.36 (d, J = 16.2 | |
| | | | | Hz, 1H), 7.24 (d, J = 21.0 Hz, 1H), 4.17 | |
| | | | | (s, 1H), 3.94 (m, 3H), 3.70 (m, 2H), | |
| | | | | 2.76 (m, 1H), 1.99-1.70 (m, 6H), 1.63- | |
| | | | | 1.35 (m, 17H), 1.04 (m, 6H). | |
| 381 | 4.11 | 583.2 | A | 1H NMR (400 MHz, CD3OD) δ 7.97 | 22B |
| | | | | (dd, J = 10.7, 2.0 Hz, 1H), 7.92-7.83 | |
| | | | | (m, 2H), 7.79 (s, 1H), 7.62 (t, J = 8.0 | |
| | | | | Hz, 1H), 7.47 (s, 1H), 7.20 (s, 1H), | |
| | | | | 4.02 (t, J = 4.9 Hz, 2H), 3.44 (bs, 2H), | |
| | | | | 3.30 (bs, 2H), 1.68 (s, 6H), 1.61 (s, 9H). | |
| 382 | 4.46 | 508.27 | A | 1H NMR (400 MHz, DMSO-d6) δ 8.20- | 22A, 11 |
| | | | | 8.13 (m, 2H), 7.80 (s, 1H), 7.65 (s, 1H), | |
| | | | | 7.61-7.53 (m, 2H), 4.09-4.02 (m, 2H), | |
| | | | | 3.89-3.81 (m, 2H), 2.35 (s, 3H), 1.94 (s, | |
| | | | | 3H), 1.81 (s, 6H), 1.53 (s, 9H). | |
| 383 | 3.33 | 517.51 | A | 1H NMR (400 MHz, DMSO-d6) δ 8.13 | 22A |
| | | | | (d, J = 8.1 Hz, 2H), 7.75 (s, 1H), 7.58- | |
| | | | | 7.48 (m, 3H), 7.40 (t, J = 7.8 Hz, 1H), | |
| | | | | 6.44 (d, J = 7.2 Hz, 1H), 6.38 (d, J = | |
| | | | | 8.5 Hz, 1H), 4.03 (t, J = 5.6 Hz, 2H), | |
| | | | | 3.86 (s, 2H), 3.56 (t, J = 5.4 Hz, 2H), | |
| | | | | 2.28 (s, 3H), 1.51 (s, 9H), 1.49 (s, 6H). | |
| 384 | 4.11 | 563.73 | A | 1H NMR (400 MHz, DMSO-d6) δ | 22A, 11 |
| | | | | 12.58 (s, 1H), 8.57 (s, 1H), 7.45 (s, 1H), | |
| | | | | 7.16 (s, 1H), 4.17-3.93 (m, 2H), 3.78 (t, | |
| | | | | J = 5.4 Hz, 2H), 3.74 (s, 2H), 2.71 (tt, | |
| | | | | J = 11.7, 3.9 Hz, 1H), 2.58 (s, 3H), | |
| | | | | 1.87-1.64 (m, 4H), 1.57 (s, 6H), 1.50- | |
| | | | | 1.47 (m, 2H), 1.46 (s, 9H), 1.33 (td, J = | |
| | | | | 13.3, 4.0 Hz, 2H), 0.97 (s, 3H), 0.95 | |
| | | | | (s, 3H). | |
| 385 | 4.1 | 581.23 | A | 1H NMR (400 MHz, DMSO-d6) δ 12.81 | 22A, 11 |
| | | | | (s, 1H), 8.57 (d, J = 0.7 Hz, 1H), 8.17 | |
| | | | | (dd, J = 11.0, 2.1 Hz, 1H), 8.04 (ddd, | |
| | | | | J = 8.5, 2.1, 0.8 Hz, 1H), 7.84 (s, 1H), | |
| | | | | 7.72 (dd, J = 8.5, 7.8 Hz, 1H), 7.57 (s, | |
| | | | | 1H), 4.11-3.98 (m, 2H), 3.81 (dd, J = | |
| | | | | 6.7, 4.0 Hz, 2H), 3.76 (s, 2H), 2.59 (s, | |
| | | | | 3H), 1.58 (s, 6H), 1.54 (s, 9H). | |
| 386 | 4.08 | 535.47 | A | 1H NMR (400 MHz, DMSO-d6) δ 8.20- | 14 Ex. 72 |
| | | | | 8.13 (m, 2H), 7.80 (s, 1H), 7.65 (s, | |
| | | | | 1H), 7.61-7.53 (m, 2H), 4.09-4.02 (m, | |
| | | | | 2H), 3.89-3.81 (m, 2H), 2.35 (s, 3H), | |
| | | | | 1.94 (s, 3H), 1.81 (s, 6H), 1.53 (s, 9H). | |
| 387 | 4.57 | 518.53 | A | 1H NMR (400 MHz, CDCl3) δ 8.09 (d, | 22A |
| | | | | J = 7.6 Hz, 2H), 8.01-7.93 (m, 1H), | |
| | | | | 7.76-7.68 (m, 2H), 7.62-7.50 (m, | |
| | | | | 3H), 4.18 (s, 1H), 4.09 (s, 1H), 4.03- | |
| | | | | 3.90 (m, 2H), 3.81-3.65 (m, 2H), 2.61- | |
| | | | | 2.48 (m, 3H), 1.66 (s, 3H), 1.63 (s, 3H), | |
| | | | | 1.63 (s, 9H). | |
| 388 | 4.77 | 518.53 | A | 1H NMR (400 MHz, CDCl3) δ 8.19 (s, | 22A |
| | | | | 2H), 7.93 (d, J = 7.4 Hz, 2H), 7.56 (s, | |
| | | | | 1H), 7.48-7.39 (m, 3H), 4.08-4.00 (m, | |
| | | | | 2H), 3.98 (s, 2H), 3.88-3.80 (m, 2H), | |
| | | | | 2.14 (s, 3H), 1.63 (s, 6H), 1.55 (d, J = | |
| | | | | 1.2 Hz, 9H). | |
| 389 | 4.77 | 519.51 | A | 1H NMR (400 MHz, CDCl3) δ 8.33- | 22A |
| | | | | 8.13 (m, 1H), 7.98 (d, J = 8.1 Hz, 2H), | |
| | | | | 7.68-7.52 (m, 2H), 7.48 (d, J = 8.0 | |
| | | | | Hz, 2H), 4.21-4.05 (m, 3H), 4.02 (dd, | |
| | | | | J = 6.1, 5.2 Hz, 1H), 3.98 (s, 1H), 3.87- | |
| | | | | 3.77 (m, 1H), 2.55 (s, 2H), 2.44 (s, 2H), | |
| | | | | 1.68 (s, 3H), 1.63 (s, 3H), 1.58 (s, 9H). | |
| 390 | 2.9 | 518.48 | A | 1H NMR (400 MHz, CDCl3) δ 8.01 (d, | 22A |
| | | | | J = 6.6 Hz, 2H), 7.77 (s, 1H), 7.67 (s, | |
| | | | | 1H), 7.53 (d, J = 7.0 Hz, 2H), 7.27 (s, | |
| | | | | 2H), 4.24 (s, 2H), 4.19 (s, 2H), 4.14 (s, | |
| | | | | 2H), 2.81 (s, 3H), 1.70 (s, 6H), 1.60 (s, | |
| | | | | 9H). | |
| 391 | 4.42 | 566.6 | A | 1H NMR (400 MHz, DMSO-d6) δ 8.44 | 22A, 11 |
| | | | | (s, 1H), 7.72 (dd, J = 14.5, 1.8 Hz, 1H), | |
| | | | | 7.40 (s, 1H), 7.13 (s, 1H), 4.04-3.95 | |
| | | | | (m, 2H), 3.91-3.80 (m, 4H), 2.74-2.59 | |
| | | | | (m, 1H), 1.81-1.58 (m, 4H), 1.52 (s, | |
| | | | | 6H), 1.43 (s, 10H), 1.39-1.24 (m, 3H), | |
| | | | | 0.93 (d, J = 9.6 Hz, 6H). | |
| 392 | 4.01 | 563.96 | A | 1H NMR (400 MHz, DMSO-d6) δ | 22A, 11 |
| | | | | 12.81 (s, 1H), 8.57 (s, 1H), 8.16 (d, J = | |
| | | | | 8.6 Hz, 2H), 7.78 (s, 1H), 7.57 (d, J = | |
| | | | | 3.7 Hz, 2H), 7.55 (s, 1H), 4.13-3.98 (m, | |
| | | | | 2H), 3.88-3.79 (m, 2H), 3.76 (s, 2H), | |
| | | | | 2.59 (s, 3H), 1.59 (s, 6H), 1.54 (s, 9H). | |
| 393 | 4.3 | 565.52 | A | 1H NMR (400 MHz, DMSO-d6) δ 8.48- | 22A, 11 |
| | | | | 8.41 (m, 1H), 8.16-8.05 (m, 2H), 7.75 | |
| | | | | (s, 2H), 7.58-7.49 (m, 3H), 4.09-3.99 | |
| | | | | (m, 2H), 3.95-3.81 (m, 4H), 1.59-1.43 | |
| | | | | (m, 15H). | |
| 394 | 4.41 | 517.43 | A | 1H NMR (400 MHz, DMSO-d6) δ 8.13 | 22A |
| | | | | (d, J = 8.0 Hz, 2H), 8.07 (d, J = 5.6 Hz, | |
| | | | | 1H), 7.75 (s, 1H), 7.58-7.50 (m, 3H), | |
| | | | | 7.48 (d, J = 7.3 Hz, 1H), 6.89 (dd, J = | |
| | | | | 6.7, 4.8 Hz, 1H), 3.84-3.75 (m, 2H), | |
| | | | | 3.37-3.35 (m, 2H), 3.17 (s, 2H), 2.26 | |
| | | | | (s, 3H), 1.56 (s, 6H), 1.53-1.48 (m, 9H). | |
| 395 | 3.51 | 517.43 | A | 1H NMR (400 MHz, DMSO-d6) δ 8.13 | 22A |
| | | | | (d, J = 8.5 Hz, 2H), 7.90 (s, 1H), 7.75 | |
| | | | | (s, 1H), 7.57-7.48 (m, 3H), 7.36 (d, | |
| | | | | J = 8.6 Hz, 1H), 6.53 (d, J = 8.6 Hz, | |
| | | | | 1H), 4.12-3.96 (m, 2H), 3.83 (s, 2H), | |
| | | | | 3.57-3.49 (m, 2H), 2.11 (s, 3H), 1.51 (s, | |
| | | | | 9H), 1.48 (s, 6H). | |
| 396 | 1.1 | 583.25 | K | 1H NMR (400 MHz, DMSO-d6) δ 8.45 | 22A, 11 |
| | | | | (t, J = 1.8 Hz, 1H), 8.22-8.10 (m, 1H), | |
| | | | | 8.08-7.96 (m, 1H), 7.80 (s, 1H), 7.73- | |
| | | | | 7.64 (m, 2H), 7.53 (s, 1H), 4.12-3.99 | |
| | | | | (m, 2H), 3.98-3.79 (m, 4H), 1.60- | |
| | | | | 1.38 (m, 15H). | |
| 397 | 4.52 | 563.22 | A | 1H NMR (400 MHz, DMSO-d6) δ | Ex. 73 |
| | | | | 12.80 (s, 1H), 8.16 (d, J = 8.6 Hz, 2H), | |
| | | | | 7.78 (s, 1H), 7.67-7.63 (m, 1H), 7.59- | |
| | | | | 7.50 (m, 4H), 4.05-3.84 (m, 2H), 3.50 | |
| | | | | (dd, J = 6.4, 4.0 Hz, 2H), 3.37 (s, 2H), | |
| | | | | 2.36 (s, 3H), 1.60 (s, 6H), 1.54 (s, 9H). | |
| 398 | 4.14 | 562.38 | A | 1H NMR (400 MHz, DMSO-d6) δ | 22A, 11 |
| | | | | 12.40 (s, 1H), 8.58 (s, 1H), 7.40 (s, | |
| | | | | 1H), 7.13 (s, 1H), 6.48 (s, 1H), 4.09- | |
| | | | | 4.00 (m, 2H), 3.94 (s, 2H), 3.62 (t, J = | |
| | | | | 5.6 Hz, 2H), 2.74-2.62 (m, 1H), 1.83- | |
| | | | | 1.63 (m, 4H), 1.48 (s, 6H), 1.47-1.45 | |
| | | | | (m, 2H), 1.44 (s, 12H), 1.31 (td, J = | |
| | | | | 13.2, 3.9 Hz, 2H), 0.95 (s, 3H), 0.92 | |
| | | | | (s, 3H). | |
| 399 | 3.98 | 563.22 | C | 1H NMR (400 MHz, DMSO-d6) δ 12.29 | 22A, 11 |
| | | | | (s, 1H), 8.59 (s, 1H), 8.13 (d, J = 8.6 | |
| | | | | Hz, 2H), 7.75 (s, 1H), 7.54 (d, J = 8.7 | |
| | | | | Hz, 2H), 7.52 (s, 1H), 6.49 (s, 1H), | |
| | | | | 4.08 (t, J = 5.6 Hz, 2H), 3.96 (s, 2H), | |
| | | | | 3.64 (t, J = 5.5 Hz, 2H), 1.51 (s, 12H), | |
| | | | | 1.50 (s, 6H). | |
| 400 | 4.11 | 581.23 | A | 1H NMR (400 MHz, DMSO-d6) δ | 22A, 11 |
| | | | | 12.51-12.11 (m, 1H), 8.62 (s, 1H), 8.18 | |
| | | | | (dd, J = 11.0, 2.1 Hz, 1H), 8.04 (dd, J = | |
| | | | | 8.5, 2.1 Hz, 1H), 7.84 (s, 1H), 7.72 (t, | |
| | | | | J = 8.1 Hz, 1H), 7.56 (s, 1H), 6.52 (s, | |
| | | | | 1H), 4.11 (t, J = 5.5 Hz, 2H), 3.99 (s, | |
| | | | | 2H), 3.75-3.62 (m, 2H), 1.55 (s, 12H), | |
| | | | | 1.53 (s, 6H). | |
| 401 | 4.45 | 564.24 | A | 1H NMR (400 MHz, DMSO-d6) δ | 22A, 11 |
| | | | | 13.30 (s, 1H), 8.13 (d, J = 8.6 Hz, 2H), | |
| | | | | 7.75 (s, 1H), 7.53 (d, J = 8.6 Hz, 2H), | |
| | | | | 7.51 (s, 1H), 7.03 (s, 1H), 4.03 (dd, J = | |
| | | | | 11.5, 5.3 Hz, 4H), 3.75 (dd, J = 8.0, | |
| | | | | 3.6 Hz, 2H), 2.37 (s, 3H), 1.51 (s, 15H). | |
| 402 | 4.72 | 504.46 | A | 1H NMR (400 MHz, DMSO-d6) δ 8.16- | 22A |
| | | | | 8.13 (m, 2H), 8.12 (s, 1H), 8.05 (dd, | |
| | | | | J = 2.7, 1.5 Hz, 1H), 7.80 (d, J = 2.6 | |
| | | | | Hz, 1H), 7.75 (s, 1H), 7.55 (s, 1H), | |
| | | | | 7.54-7.51 (m, 2H), 4.12-4.03 (m, 2H), | |
| | | | | 3.90 (s, 2H), 3.69-3.62 (m, 2H), 1.51 | |
| | | | | (s, 9H), 1.51 (s, 6H). | |
| 403 | 5.19 | 518.5 | A | 1H NMR (400 MHz, DMSO-d6) δ 8.21 | 22A |
| | | | | (d, J = 4.9 Hz, 1H), 8.13 (d, J = 8.8 Hz, | |
| | | | | 2H), 7.75 (s, 1H), 7.54 (d, J = 8.7 Hz, | |
| | | | | 2H), 7.51 (s, 1H), 6.53 (d, J = 5.0 Hz, | |
| | | | | 1H), 4.08-3.98 (m, 2H), 3.96 (s, 2H), | |
| | | | | 3.77-3.63 (m, 2H), 2.27 (s, 3H), 1.51 | |
| | | | | (s, 9H), 1.49 (s, 6H). | |
| 404 | 4.97 | 504.46 | A | 1H NMR (400 MHz, DMSO-d6) δ 8.36 | 22A |
| | | | | (dd, J = 4.7, 1.0 Hz, 2H), 8.13 (d, J = | |
| | | | | 7.9 Hz, 2H), 7.75 (s, 1H), 7.54 (d, J = | |
| | | | | 7.8 Hz, 2H), 7.51 (d, J = 1.1 Hz, 1H), | |
| | | | | 6.64 (t, J = 4.7 Hz, 1H), 4.10-3.99 (m, | |
| | | | | 2H), 3.97 (s, 2H), 3.75-3.62 (m, 2H), | |
| | | | | 1.51 (s, 9H), 1.50 (s, 6H). | |
| 405 | 4.54 | 579.4 | A | 1H NMR (400 MHz, DMSO-d6) δ 8.98 | 1, 11 |
| | | | | 8.92 (m, 1H), 8.31 (dd, J = 8.8, 2.3 Hz, | |
| | | | | 1H), 8.19 (dd, J = 11.0, 2.1 Hz, 1H), | |
| | | | | 8.12 (d, J = 8.6 Hz, 1H), 8.08-8.01 (m, | |
| | | | | 1H), 7.86 (s, 1H), 7.77-7.68 (m, 1H), | |
| | | | | 7.66 (s, 1H), 4.35-4.22 (m, 2H), 4.14- | |
| | | | | 3.97 (m, 2H), 1.86 (s, 6H), 1.55 (s, 9H). | |
| 406 | 4.35 | 524.48 | A | 1H NMR (400 MHz, DMSO-d6) δ | Example 74 |
| | | | | 12.59 (broad s, 1H), 8.18 (dd, J = 11.0, | |
| | | | | 2.0 Hz, 1H), 8.04 (dd, J = 8.5, 1.8 Hz, | |
| | | | | 1H), 7.85 (s, 1H), 7.76-7.66 (m, 2H), | |
| | | | | 7.65 (s, 1H), 6.80-6.57 (m, 1H), 4.22- | |
| | | | | 4.07 (m, 2H), 4.04-3.88 (m, 2H), 1.80 | |
| | | | | (s, 6H), 1.54 (s, 9H). | |
| 407 | 4.2 | 506.46 | A | 1H NMR (400 MHz, DMSO-d6) δ | 1, 21 |
| | | | | 12.60 (broad s, 1H), 8.16 (d, J = 8.6 Hz, | |
| | | | | 2H), 7.79 (s, 1H), 7.71 (s, 1H), 7.64 (s, | |
| | | | | 1H), 7.56 (d, J = 8.6 Hz, 2H), 6.71 (s, | |
| | | | | 1H), 4.21-4.07 (m, 2H), 4.06-3.89 (m, | |
| | | | | 2H), 1.80 (s, 6H), 1.53 (s, 9H). | |
| 408 | 4.56 | 563.73 | A | 1H NMR (400 MHz, DMSO-d6) δ | 1, 21 |
| | | | | 13.40 (s, 1H), 7.40 (s, 1H), 7.13 (s, | |
| | | | | 1H), 7.02 (s, 1H), 4.33-3.91 (m, 4H), | |
| | | | | 3.73 (dd, J = 7.0, 4.5 Hz, 2H), 2.78- | |
| | | | | 2.62 (m, 1H), 2.36 (s, 3H), 1.82-1.60 | |
| | | | | (m, 4H), 1.49 (s, 6H), 1.46-1.44 (m, | |
| | | | | 2H), 1.43 (s, 9H), 1.30 (td, J = 13.1, | |
| | | | | 4.1 Hz, 2H), 0.94 (s, 3H), 0.92 (s, 3H). | |
| 409 | 4.56 | 582.26 | C | 1H NMR (400 MHz, DMSO-d6) δ 8.15 | 22A, 11 |
| | | | | (dd, J = 11.0, 2.1 Hz, 1H), 8.05-7.94 | |
| | | | | (m, 1H), 7.81 (s, 1H), 7.69 (t, J = 8.1 | |
| | | | | Hz, 1H), 7.52 (s, 1H), 7.03 (s, 1H), | |
| | | | | 4.03 (dd, J = 11.0, 4.7 Hz, 4H), 3.75 | |
| | | | | (dd, J = 7.0, 4.3 Hz, 2H), 2.37 (s, 3H), | |
| | | | | 1.51 (s, 9H), 1.51 (s, 6H). | |
| 410 | 4.31 | 561.46 | A | 1H NMR (400 MHz, DMSO-d6) δ | 22A, 11 |
| | | | | 12.27 (s, 1H), 8.18-8.08 (m, 2H), 7.96 | |
| | | | | (d, J = 8.9 Hz, 1H), 7.75 (s, 1H), 7.58- | |
| | | | | 7.49 (m, 3H), 6.51 (d, J = 9.0 Hz, 1H), | |
| | | | | 4.15-4.04 (m, 2H), 3.96 (s, 2H), 3.72- | |
| | | | | 3.58 (m, 2H), 2.59 (s, 3H), 1.57-1.41 | |
| | | | | (m, 15H). | |
| 411 | 4.44 | 579.48 | A | 1H NMR (400 MHz, DMSO-d6) δ 8.15 | 22A, 11 |
| | | | | (dd, J = 11.0, 2.0 Hz, 1H), 8.01 (dd, | |
| | | | | J = 8.6, 2.1 Hz, 1H), 7.96 (d, J = 8.9 | |
| | | | | Hz, 1H), 7.81 (s, 1H), 7.69 (t, J = 8.2 | |
| | | | | Hz, 1H), 7.53 (s, 1H), 6.51 (d, J = 8.9 | |
| | | | | Hz, 1H), 4.13-4.00 (m, 2H), 3.96 (s, | |
| | | | | 2H), 3.71-3.55 (m, 2H), 2.59 (s, 3H), | |
| | | | | 1.57-1.41 (m, 15H). | |
| 412 | 3.14 | 522.19 | A | 1H NMR (400 MHz, CDCl3) δ 8.72 (s, | 22A |
| | | | | 1H), 8.35 (s, 1H), 7.84 (dd, J = 10.1, | |
| | | | | 2.0 Hz, 1H), 7.79-7.72 (m, 1H), 7.61 | |
| | | | | (s, 1H), 7.58 (s, 1H), 7.52 (t, J = 7.8 | |
| | | | | Hz, 1H), 6.87 (d, J = 85.5 Hz, 1H), | |
| | | | | 4.26 (s, 2H), 4.12 (d, J = 29.7 Hz, 2H), | |
| | | | | 3.84 (s, 2H), 1.69 (d, J = 12.0 Hz, 6H), | |
| | | | | 1.57 (s, 9H). | |
| 413 | 4.09 | 548.61 | A | 1H NMR (400 MHz, DMSO-d6) δ | 22A, 11 |
| | | | | 12.54 (s, 1H, br), 8.65-8.63 (m, 1H), | |
| | | | | 8.01-7.93 (m, 1H), 7.44 (s, 1H), 7.16 | |
| | | | | (s, 1H), 6.71-6.64 (m, 1H), 4.13-4.05 | |
| | | | | (m, 2H), 4.00 (s, 2H), 3.69-3.62 (m, | |
| | | | | 2H), 2.79-2.62 (m, 1H), 1.86-1.65 | |
| | | | | (m, 4H), 1.52 (s, 6H), 1.47 (s, 11H), | |
| | | | | 1.40-1.28 (m, 2H), 0.98 (s, 3H), 0.96 | |
| | | | | (s, 3H). | |
| 414 | 2.99 | 548.75 | A | 1H NMR (400 MHz, DMSO-d6) δ 8.29 | 22A |
| | | | | (d, J = 6.2 Hz, 1H), 7.41 (s, 1H), 7.13 | |
| | | | | (s, 1H), 4.06 (s, 2H), 3.85-3.55 (m, | |
| | | | | 4H), 2.84-2.61 (m, 1H), 1.85-1.60 | |
| | | | | (m, 4H), 1.49 (s, 6H), 1.47-1.44 (m, | |
| | | | | 2H), 1.44 (s, 9H), 1.30 (td, J = 13.2, | |
| | | | | 4.0 Hz, 3H), 0.95 (s, 3H), 0.92 (s, 3H). | |
| 415 | 2.95 | 549.26 | A | 1H NMR (400 MHz, DMSO-d6) δ 8.33 | 22A |
| | | | | (d, J = 6.2 Hz, 1H), 8.21-8.08 (m, 2H), | |
| | | | | 7.79 (s, 1H), 7.57 (d, J = 8.4 Hz, 3H), | |
| | | | | 6.91 (d, J = 161.0 Hz, 1H), 4.13 (s, | |
| | | | | 2H), 3.88-3.61 (m, 4H), 1.54 (s, 15H). | |
| 416 | 4.07 | 555.88 | A | 1H NMR (400 MHz, DMSO-d6) δ 7.39 | 1, 11 |
| | | | | (d, J = 17.2 Hz, 1H), 7.12 (d, J = 2.5 | |
| | | | | Hz, 1H), 3.94-3.80 (m, 2H), 3.72-3.54 | |
| | | | | (m, 3H), 3.43 (t, J = 5.8 Hz, 1H), 2.72- | |
| | | | | 2.62 (m, 1H), 2.55 (d, J = 23.0 Hz, | |
| | | | | 2H), 1.81-1.57 (m, 4H), 1.56-1.36 (m, | |
| | | | | 18H), 1.36-1.23 (m, 2H), 1.14 (s, 6H), | |
| | | | | 0.99-0.85 (m, 6H). | |
| 417 | 4.05 | 573.57 | A | 1H NMR (400 MHz, DMSO-d6) δ | 1, 11 |
| | | | | 11.55 (bs, 1H), 8.14 (dt, J = 11.1, 1.7 | |
| | | | | Hz, 1H), 8.01 (d, J = 8.5 Hz, 1H), 7.80 | |
| | | | | (d, J = 2.7 Hz, 1H), 7.69 (td, J = 8.2, 2.2 | |
| | | | | Hz, 1H), 7.51 (d, J = 15.3 Hz, 1H), | |
| | | | | 4.01-3.84 (m, 2H), 3.73-3.55 (m, 3H), | |
| | | | | 3.55-3.43 (m, 1H), 2.57 (d, J = 23.2 Hz, | |
| | | | | 2H), 1.57-1.37 (m, 15H), 1.15 (d, J = | |
| | | | | 2.1 Hz, 6H). | |
| 418 | 3.7 | 527.04 | A | 1H NMR (400 MHz, DMSO-d6) δ 7.41 | 1, 23 |
| | | | | (d, J = 10.1 Hz, 1H), 7.19 (s, 1H), 3.97- | |
| | | | | 3.81 (m, 2H), 3.70-3.65 (m, 1H), 3.62 | |
| | | | | (s, 2H), 3.50-3.42 (m, 1H), 2.71 (tt, | |
| | | | | J = 11.8, 3.8 Hz, 1H), 2.58-2.51 (m, | |
| | | | | 1H), 2.46-2.39 (m, 2H), 1.83-1.57 (m, | |
| | | | | 4H), 1.52-1.40 (m, 18H), 1.40-1.22 | |
| | | | | (m, 2H), 0.94 (d, J = 11.3 Hz, 6H). | |
| 419 | 3.69 | 545.48 | A | 1H NMR (400 MHz, DMSO-d6) δ 8.14 | 1, 23 |
| | | | | (dt, J = 10.9, 1.7 Hz, 1H), 8.01 (dd, J = | |
| | | | | 8.5, 1.7 Hz, 1H), 7.80 (d, J = 1.8 Hz, | |
| | | | | 1H), 7.69 (td, J = 8.2, 1.3 Hz, 1H), 7.51 | |
| | | | | (d, J = 10.6 Hz, 1H), 4.00-3.83 (m, | |
| | | | | 2H), 3.76-3.67 (m, 1H), 3.63 (d, J = | |
| | | | | 1.8 Hz, 2H), 3.52-3.45 (m, 1H), 2.61- | |
| | | | | 2.52 (m, 1H), 1.57-1.37 (m, 15H). | |
| 420 | 3.58 | 526.48 | A | 1H NMR (400 MHz, DMSO-d6) δ 8.18- | 1, 23 |
| | | | | 8.06 (m, 2H), 7.75 (d, J = 1.8 Hz, 1H), | |
| | | | | 7.59-7.45 (m, 3H), 3.96-3.86 (m, | |
| | | | | 2H), 3.74-3.67 (m, 1H), 3.63 (s, 2H), | |
| | | | | 3.56-3.44 (m, 1H), 2.62-2.50 (m, | |
| | | | | 1H), 1.58-1.38 (m, 15H). | |
| 421 | 3.79 | 533.63 | A | 1H NMR (400 MHz, DMSO-d6) δ | 22A, 11 |
| | | | | 12.66 (s, 1H), 8.88-8.64 (m, 2H), 8.20- | |
| | | | | 8.06 (m, 2H), 7.72 (s, 1H), 7.51 (s, | |
| | | | | 1H), 7.30 (t, J = 8.8 Hz, 2H), 4.07 (d, | |
| | | | | J = 5.3 Hz, 4H), 3.78 (dd, J = 7.2, 4.3 | |
| | | | | Hz, 2H), 1.51 (s, 6H), 1.51 (s, 9H). | |
| 422 | 4.1 | 612.56 | A | 1H NMR (400 MHz, DMSO-d6) δ 8.14 | 3 |
| | | | | (dt, J = 11.0, 2.2 Hz, 1H), 8.01 (dt, J = | |
| | | | | 8.5, 2.2 Hz, 1H), 7.80 (d, J = 3.8 Hz, | |
| | | | | 1H), 7.69 (td, J = 8.2, 2.3 Hz, 1H), | |
| | | | | 7.51 (d, J = 15.0 Hz, 1H), 3.99-3.83 | |
| | | | | (m, 2H), 3.79-3.53 (m, 3H), 3.49-3.40 | |
| | | | | (m, 1H), 2.10-1.97 (m, 2H), 1.70- | |
| | | | | 1.28 (m, 20H), 1.28-0.99 (m, 5H). | |
| 423 | 3.99 | 594.6 | A | 1H NMR (400 MHz, DMSO-d6) δ | 3 |
| | | | | 12.00 (bs, 1H), 8.22-8.06 (m, 2H), | |
| | | | | 7.75 (d, J = 3.7 Hz, 1H), 7.64-7.39 | |
| | | | | (m, 3H), 3.97-3.84 (m, 2H), 3.79- | |
| | | | | 3.56 (m, 3H), 3.55-3.40 (m, 1H), 2.13- | |
| | | | | 1.97 (m, 2H), 1.67-1.28 (m, 20H), | |
| | | | | 1.28-0.79 (m, 5H). | |
| 424 | 4.08 | 573.25 | A | 1H NMR (400 MHz, DMSO-d6) δ 8.15 | 22A, 11 |
| | | | | (dd, J = 11.0, 2.1 Hz, 1H), 8.01 (ddd, | |
| | | | | J = 8.5, 2.1, 0.7 Hz, 1H), 7.81 (s, 1H), | |
| | | | | 7.69 (t, J = 8.1 Hz, 1H), 7.54 (s, 1H), | |
| | | | | 7.46 (s, 1H), 4.05 (t, J = 5.5 Hz, 2H), | |
| | | | | 3.73 (s, 2H), 3.57 (t, J = 5.5 Hz, 2H), | |
| | | | | 1.53 (s, 6H), 1.51 (s, 9H). | |
| 425 | 4.13 | 551.64 | A | 1H NMR (400 MHz, DMSO-d6) δ | 22A, 11 |
| | | | | 12.56 (s, 1H), 7.40 (s, 1H), 7.13 (s, | |
| | | | | 1H), 3.97-3.80 (m, 2H), 3.62-3.51 | |
| | | | | (m, 4H), 2.73-2.62 (m, 1H), 2.43 (s, | |
| | | | | 3H), 1.82-1.61 (m, 4H), 1.50 (s, 6H), | |
| | | | | 1.47-1.44 (m, 2H), 1.43 (s, 9H), 1.34- | |
| | | | | 1.27 (m, 2H), 0.94 (s, 3H), 0.92 (s, 3H). | |
| 426 | 4.01 | 555.28 | A | 1H NMR (400 MHz, DMSO-d6) δ 8.13 | 22A, 11 |
| | | | | (d, J = 8.6 Hz, 2H), 7.75 (s, 1H), 7.58- | |
| | | | | 7.48 (m, 3H), 7.40 (s, 1H), 4.05 (dd, | |
| | | | | J = 6.3, 4.7 Hz, 2H), 3.72 (s, 2H). 3.57 | |
| | | | | (dd, J = 6.3, 4.7 Hz, 2H), 1.53 (s, 6H), | |
| | | | | 1.51 (s, 9H). | |
| 427 | 4.189 | 549.45 | A | 1H NMR (400 MHz, DMSO-d6) δ | 22A, 11 |
| | | | | 12.65 (s, 1H), 8.90-8.59 (m, 2H), 7.40 | |
| | | | | (s, 1H), 7.13 (s, 1H), 4.14-3.98 (m, | |
| | | | | 4H), 3.82-3.66 (m, 2H), 2.72-2.62 | |
| | | | | (m, 1H), 1.84-1.61 (m, 4H), 1.50 (s, | |
| | | | | 6H), 1.47-1.45 (m, 2H), 1.43 (s, 9H), | |
| | | | | 1.30 (td, J = 13.2, 4.0 Hz, 2H), 0.94 (s, | |
| | | | | 3H), 0.92 (s, 3H). | |
| 428 | 4.1 | 566.5 | A | 1H NMR (400 MHz, DMSO-d6) δ | 22A, 11 |
| | | | | 12.88 (s, 1H), 8.82 (d, J = 5.5 Hz, 2H), | |
| | | | | 8.18 (dd, J = 11.0, 2.1 Hz, 1H), 8.10- | |
| | | | | 7.98 (m, 1H), 7.84 (s, 1H), 7.72 (dd, | |
| | | | | J = 8.5, 7.8 Hz, 1H), 7.56 (s, 1H), 4.27- | |
| | | | | 4.04 (m, 4H), 3.91-3.69 (m, 2H), 1.54 | |
| | | | | (s, 15H). | |
| 429 | 3.99 | 548.49 | A | 1H NMR (400 MHz, DMSO-d6) δ | 22A, 11 |
| | | | | 12.85 (d, J = 35.9 Hz, 1H), 8.88-8.77 | |
| | | | | (m, 2H), 8.16 (d, J = 8.6 Hz, 2H), 7.78 | |
| | | | | (s, 1H), 7.57 (s, 2H), 7.55 (d, J = 2.7 | |
| | | | | Hz, 1H), 4.16-4.04 (m, 4H), 3.88- | |
| | | | | 3.73 (m, 2H), 1.54 (s, 6H), 1.54 (s, 9H). | |
| 430 | 3.97 | 569.49 | A | 1H NMR (400 MHz, DMSO-d6) δ | 22A, 11 |
| | | | | 12.53 (s, 1H), 8.15 (dd, J = 11.0, 2.1 | |
| | | | | Hz, 1H), 8.05-7.94 (m, 1H), 7.81 (s, | |
| | | | | 1H), 7.73-7.64 (m, 1H), 7.53 (s, 1H), | |
| | | | | 3.96 (t, J = 5.4 Hz, 2H), 3.60 (s, 3H), | |
| | | | | 3.13 (d, J = 4.8 Hz, 1H), 2.81-2.56 | |
| | | | | (m, 1H), 2.43 (s, 2H), 1.51 (s, 6H), | |
| | | | | 1.51 (s, 9H). | |
| 431 | 3.85 | 551.48 | A | 1H NMR (400 MHz, DMSO-d6) δ | 22A, 11 |
| | | | | 12.61 (d, J = 22.7 Hz, 1H), 8.13 (d, J = | |
| | | | | 8.6 Hz, 2H), 7.75 (s, 1H), 7.64-7.43 | |
| | | | | (m, 3H), 3.96 (t, J = 5.5 Hz, 2H), 3.60 | |
| | | | | (s, 3H), 2.66-2.61 (m, 1H), 2.43 (s, | |
| | | | | 2H), 2.32-2.27 (m, 1H), 1.52 (s, 6H), | |
| | | | | 1.51 (s, 9H). | |
| 432 | 4.15 | 525.5 | A | 1H NMR (400 MHz, DMSO-d6) δ 8.17 | 1 |
| | | | | (dd, J = 11.0, 2.1 Hz, 1H), 8.03 (ddd, | |
| | | | | J = 8.5, 2.1, 0.7 Hz, 1H), 7.82 (s, 1H), | |
| | | | | 7.72 (dd, J = 8.5, 7.8 Hz, 1H), 7.49 (s, | |
| | | | | 1H), 3.83-3.67 (m, 1H), 3.26-3.18 | |
| | | | | (m, 1H), 2.27 (d, J = 6.7 Hz, 2H), 2.17 | |
| | | | | (s, 1H), 1.91-1.81 (m, 1H), 1.58 (dd, | |
| | | | | J = 14.2, 3.9 Hz, 1H), 1.54 (s, 3H), 1.53 | |
| | | | | (s, 9H), 1.47 (s, 1H), 1.44 (s, 3H), 1.37- | |
| | | | | 1.26 (m, 1H). | |
| 433 | 2.81 | 632.43 | A | 1H NMR (400 MHz, DMSO-d6) δ 8.17 | Ex. 69 |
| | | | | (dd, J = 11.0, 2.1 Hz, 1H), 8.03 (ddd, | |
| | | | | J = 8.5, 2.1, 0.7 Hz, 1H), 7.81 (s, 1H), | |
| | | | | 7.71 (dd, J = 8.5, 7.8 Hz, 1H), 7.47 (s, | |
| | | | | 1H), 3.80-3.69 (m, 1H), 3.27-3.16 (m, | |
| | | | | 1H), 2.16-2.07 (m, 1H), 2.04-1.97 (m, | |
| | | | | 2H), 1.91-1.81 (m, 1H), 1.63-1.57 (m, | |
| | | | | 1H), 1.53 (d, J = 1.5 Hz, 12H), 1.43 | |
| | | | | (s, 3H), 1.41-1.33 (m, 1H), 1.32-1.21 | |
| | | | | (m, 1H). | |
| 434 | 3.42 | 565.59 | A | 1H NMR (400 MHz, DMSO-d6) δ 7.24 | Ex. 69 |
| | | | | (d, J = 12.1 Hz, 1H), 6.59 (d, J = 2.0 | |
| | | | | Hz, 1H), 5.19-5.06 (m, 1H), 5.00 (d, | |
| | | | | J = 18.4 Hz, 1H), 3.92-3.84 (m, 2H), | |
| | | | | 3.79-3.69 (m, 1H), 3.65-3.59 (m, 2H), | |
| | | | | 3.53 (s, 1H), 3.51-3.45 (m, 2H), 2.94- | |
| | | | | 2.76 (m, 1H), 2.20-2.04 (m, 7H), 1.52- | |
| | | | | 1.38 (m, 15H), 1.27 (d, J = 10.8 Hz, 3H). | |
| 435 | 2.96 | 537.59 | A | 1H NMR (400 MHz, DMSO-d6) δ 7.17 | Ex. 69 |
| | | | | (d, J = 11.5 Hz, 1H), 6.72 (d, J = 2.1 | |
| | | | | Hz, 1H), 5.00 (d, J = 18.5 Hz, 1H), | |
| | | | | 3.92-3.84 (m, 2H), 3.60 (d, J = 6.6 Hz, | |
| | | | | 6H), 3.53 (s, 1H), 3.51-3.44 (m, 1H), | |
| | | | | 2.92-2.78 (m, 1H), 2.22-2.04 (m, 4H), | |
| | | | | 1.48-1.38 (m, 19H), 1.27 (d, J = 11.0 | |
| | | | | Hz, 3H), 0.34 (s, 4H). | |
| 436 | 2.54 | 523.59 | A | 1H NMR (400 MHz, DMSO-d6) δ 7.18 | Ex. 69 |
| | | | | (d, J = 12.7 Hz, 1H), 6.20 (d, J = 2.0 | |
| | | | | Hz, 1H), 5.00 (d, J = 18.4 Hz, 1H), | |
| | | | | 3.93 (d, J = 1.2 Hz, 4H), 3.91-3.84 (m, | |
| | | | | 2H), 3.65-3.58 (m, 2H), 3.53 (s, 1H), | |
| | | | | 3.49-3.45 (m, 1H), 2.92-2.77 (m, 1H), | |
| | | | | 2.17 (t, J = 7.7 Hz, 6H), 2.12-2.05 (m, | |
| | | | | 2H), 1.82 (p, J = 8.2, 7.8 Hz, 2H), 1.50- | |
| | | | | 1.37 (m, 15H), 1.27 (d, J = 11.0 Hz, 3H). | |
| 437 | 3.3 | 579.63 | A | 1H NMR (400 MHz, DMSO-d6) δ 7.19 | Ex. 69 |
| | | | | (d, J = 11.4 Hz, 1H), 6.75 (d, J = 2.1 | |
| | | | | Hz, 1H), 5.00 (d, J = 18.6 Hz, 1H), 4.40 | |
| | | | | (d, J = 13.5 Hz, 2H), 3.92-3.84 (m, | |
| | | | | 2H), 3.65-3.43 (m, 4H), 1.96-1.86 (m, | |
| | | | | 2H), 2.86 (t, J = 11.7 Hz, 2H), 2.65-2.53 | |
| | | | | (m, 1H), 2.23-2.04 (m, 4H), 1.50-1.40 | |
| | | | | (m, 15H), 1.27 (d, J = 11.1 Hz, 3H). | |
| 438 | 2.58 | 536.61 | A | 1H NMR (400 MHz, DMSO-d6) δ 7.19 | Ex. 69 |
| | | | | (d, J = 11.4 Hz, 1H), 6.74 (d, J = 2.2 | |
| | | | | Hz, 1H), 5.00 (d, J = 18.7 Hz, 1H), | |
| | | | | 3.93-3.75 (m, 4H), 3.65-3.59 (m, 2H), | |
| | | | | 3.53 (s, 1H), 3.50-3.45 (m, 1H), 3.15- | |
| | | | | 3.04 (m, 1H), 2.94-2.77 (m, 1H), 2.22- | |
| | | | | 2.05 (m, 4H), 2.00-1.89 (m, 2H), | |
| | | | | 1.76 (dd, J = 8.2, 4.3 Hz, 2H), 1.50-1.40 | |
| | | | | (m, 15H), 1.27 (d, J = 11.0 Hz, 3H). | |
| 439 | 2.86 | 533.53 | A | 1H NMR (400 MHz, DMSO-d6) δ 7.22 | Ex. 69 |
| | | | | (d, J = 12.3 Hz, 1H), 6.41 (d, J = 1.9 | |
| | | | | Hz, 1H), 5.00 (d, J = 18.5 Hz, 1H), | |
| | | | | 3.95-3.82 (m, 4H), 3.71-3.59 (m, 4H), | |
| | | | | 3.53 (s, 1H), 3.51-3.45 (m, 1H), 2.93- | |
| | | | | 2.75 (m, 1H), 2.62-2.52 (m, 2H), 2.22- | |
| | | | | 2.04 (m, 4H), 1.51-1.40 (m, 15H), | |
| | | | | 1.27 (d, J = 11.1 Hz, 3H). | |
| 440 | 3.42 | 565.54 | A | 1H NMR (400 MHz, DMSO-d6) δ 7.24 | Ex. 69 |
| | | | | (d, J = 12.1 Hz, 1H), 6.59 (d, J = 2.0 | |
| | | | | Hz, 1H), 5.171-5.05 (m, 1H), 5.00 (d, | |
| | | | | J = 18.4 Hz, 1H), 3.93-3.83 (m, 2H), | |
| | | | | 3.79-3.69 (m, 1H), 3.61 (s, 2H), 3.53 | |
| | | | | (s, 1H), 3.50-3.45 (m, 2H), 2.93-2.77 | |
| | | | | (m, 1H), 2.21-1.99 (m, 7H), 1.52-1.41 | |
| | | | | (m, 15H), 1.27 (d, J = 10.9 Hz, 3H). | |
| 441 | 2.62 | 559.52 | A | 1H NMR (400 MHz, DMSO-d6) δ 7.20 | Ex. 69 |
| | | | | (d, J = 12.7 Hz, 1H), 6.24 (d, J = 1.8 | |
| | | | | Hz, 1H), 4.99 (d, J = 18.7 Hz, 1H), 4.07 | |
| | | | | (s, 4H), 3.92-3.80 (m, 2H), 3.66-3.59 | |
| | | | | (m, 2H), 3.50-3.43 (m, 1H), 3.53 (s, | |
| | | | | 1H), 2.92-2.79 (m, 5H), 2.23-2.02 | |
| | | | | (m, 4H), 1.49-1.39 (m, 15H), 1.27 (d, | |
| | | | | J = 11.2 Hz, 3H). | |
| 442 | 2.29 | 569.65 | A | 1H NMR (400 MHz, DMSO-d6) δ 7.14 | Ex. 69 |
| | | | | (d, J = 11.6 Hz, 1H), 6.67 (d, J = 2.1 | |
| | | | | Hz, 1H), 4.97 (d, J = 18.4 Hz, 1H), 4.33 | |
| | | | | (d, J = 12.9 Hz, 2H), 4.10 (s, 1H), 3.90- | |
| | | | | 3.79 (m, 2H), 3.65-3.55 (m, 2H), 3.55- | |
| | | | | 3.41 (m, 2H), 2.91-2.74 (m, 1H), 2.66 | |
| | | | | (t, J = 12.0 Hz, 2H), 2.19-1.98 (m, 4H), | |
| | | | | 1.74 (d, J = 11.3 Hz, 2H), 1.46-1.36 (m, | |
| | | | | 15H), 1.24 (d, J = 10.9 Hz, 3H), 1.02 | |
| | | | | (s, 6H). | |
| 443 | 3.91 | 531.57 | A | 1H NMR (400 MHz, DMSO-d6) δ 3.62- | 22A, 11 |
| | | | | 3.54 (m, 1H), 1.45 (s, 9H), 2.39-2.25 | |
| | | | | (m, 4H), 2.17-2.09 (m, 2H), 1.96- | |
| | | | | 1.76 (m, 4H), 1.52 (s, 6H), 3.70-3.63 | |
| | | | | (m, 2H), 8.63 (d, J = 2.3 Hz, 1H), 7.97 | |
| | | | | (dd, J = 8.9, 2.3 Hz, 1H), 7.45 (s, 1H), | |
| | | | | 7.10 (s, 1H), 6.66 (d, J = 9.1 Hz, 1H), | |
| | | | | 4.11-4.05 (m, 2H), 3.99 (s, 2H). | |
| 444 | 2.61 | 523.54 | A | 1H NMR (400 MHz, DMSO-d6) δ 7.43 | 3 |
| | | | | (d, J = 8.6 Hz, 1H), 7.10 (d, J = 2.2 Hz, | |
| | | | | 1H), 4.17 (ddd, J = 15.0, 9.0, 3.2 Hz, | |
| | | | | 1H), 3.95-3.46 (m, 9H), 2.88-2.61 | |
| | | | | (m, 4H), 2.39-2.22 (m, 4H), 2.12 (t, | |
| | | | | J = 7.1 Hz, 2H), 1.91 (t, J = 7.0 Hz, | |
| | | | | 2H), 1.82 (q, J = 7.9 Hz, 2H), 1.51 (s, | |
| | | | | 3H), 1.49-1.40 (m, 12H). | |
| 445 | 2.55 | 523.59 | A | 1H NMR (400 MHz, DMSO-d6) d7.40 | 3 |
| | | | | (d, J = 8.6 Hz, 1H), 7.07 (d, J = 2.2 Hz, | |
| | | | | 1H), 4.14 (ddd, J = 15.0, 8.9, 3.1 Hz, | |
| | | | | 1H), 3.92-3.38 (m, 9H), 2.85-2.58 | |
| | | | | (m, 4H), 2.35-2.20 (m, 4H), 2.09 (t, | |
| | | | | J = 7.1 Hz, 2H), 1.88 (t, J = 6.9 Hz, | |
| | | | | 2H), 1.84-1.73 (m, 2H), 1.48 (s, 3H), | |
| | | | | 1.46-1.38 (m, 12H). | |
| 446 | 2.62 | 523.54 | A | 1H NMR (400 MHz, DMSO-d6) δ 7.40 | 3 |
| | | | | (d, J = 14.5 Hz, 1H), 7.07 (d, J = 2.5 | |
| | | | | Hz, 1H), 4.02-3.35 (m, 12H), 2.80- | |
| | | | | 2.64 (m, 2H), 2.35-2.19 (m, 4H), 2.09 | |
| | | | | (t, J = 7.2 Hz, 2H), 1.88 (t, J = 6.9 Hz, | |
| | | | | 2H), 1.79 (q, J = 7.8 Hz, 2H), 1.49 (d, | |
| | | | | J = 1.7 Hz, 3H), 1.46-1.36 (m, 12H). | |
| 447 | 2.63 | 523.59 | A | 1H NMR (400 MHz, DMSO-d6) δ 7.40 | 3 |
| | | | | (d, J = 14.5 Hz, 1H), 7.07 (d, J = 2.5 | |
| | | | | Hz, 1H), 3.99-3.41 (m, 12H), 2.82- | |
| | | | | 2.62 (m, 2H), 2.34-2.20 (m, 4H), 2.09 | |
| | | | | (t, J = 7.2 Hz, 2H), 1.92-1.84 (m, 2H), | |
| | | | | 1.84-1.73 (m, 2H), 1.49 (d, J = 1.8 Hz, | |
| | | | | 3H), 1.46-1.37 (m, 12H). | |
| 448 | 4.27 | 565.45 | A | 1H NMR (400 MHz, DMSO-d6) δ | 3, 11 |
| | | | | 12.57 (s, 1H), 8.15 (dd, J = 10.9, 2.1 | |
| | | | | Hz, 1H), 8.02 (dd, J = 8.6, 2.1 Hz, 1H), | |
| | | | | 7.81 (s, 1H), 7.73-7.66 (m, 2H), 7.53 | |
| | | | | (s, 1H), 7.27 (d, J = 7.2 Hz, 1H), 6.84 | |
| | | | | (d, J = 8.6 Hz, 1H), 4.14-4.02 (m, 2H), | |
| | | | | 3.98 (s, 2H), 3.70-3.56 (m, 2H), 1.70- | |
| | | | | 1.44 (m, 15H). | |
| 449 | 4.27 | 547.02 | A | | 3, 23 |
| 450 | 4.08 | 537.96 | A | 1H NMR (400 MHz, DMSO-d6) δ | 22A, 11 |
| | | | | 12.72 (s, 1H, broad), 8.23 (s, 1H), 7.43 | |
| | | | | (s, 1H), 7.16 (s, 1H), 3.97 (t, J = 5.7 | |
| | | | | Hz, 2H), 3.69-3.60 (m, 4H), 2.77- | |
| | | | | 2.64 (m, 1H), 1.87-1.64 (m, 4H), 1.53 | |
| | | | | (s, 6H), 1.51-1.43 (m, 11H), 1.40- | |
| | | | | 1.27 (m, 2H), 0.98 (s, 3H), 0.95 (s, 3H). | |
| 451 | 4.07 | 548.58 | A | 1H NMR (400 MHz, DMSO-d6) δ | 22S |
| | | | | 12.69 (s, 1H), 8.65 (d, J = 1.3 Hz, 1H), | |
| | | | | 8.21 (s, 1H), 7.42 (s, 1H), 7.13 (s, 1H), | |
| | | | | 4.08 (t, J = 5.6 Hz, 2H), 3.99 (s, 2H), | |
| | | | | 3.74 (dd, J = 6.7, 4.4 Hz, 2H), 2.68 (tt, | |
| | | | | J = 11.6, 3.9 Hz, 1H), 1.92-1.59 (m, | |
| | | | | 4H), 1.51 (s, 6H), 1.44 (s, 9H), 1.43- | |
| | | | | 1.41 (m, 2H), 1.31 (td, J = 13.5, 4.3 Hz, | |
| | | | | 2H), 0.95 (s, 3H), 0.92 (s, 3H). | |
| 452 | 3.97 | 548.44 | A | 1H NMR (400 MHz, DMSO-d6) δ | 22A, 11 |
| | | | | 12.73 (s, 1H), 8.68 (d, J = 1.2 Hz, 1H), | |
| | | | | 8.25 (s, 1H), 8.16 (d, J = 8.6 Hz, 2H), | |
| | | | | 7.79 (s, 1H), 7.63-7.50 (m, 3H), 4.15 | |
| | | | | (dd, J = 6.7, 4.4 Hz, 2H), 4.04 (s, 2H), | |
| | | | | 3.80 (dd, J = 6.6, 4.7 Hz, 2H), 1.55 (s, | |
| | | | | 6H), 1.54 (s, 9H). | |
| 453 | 3.25 | 438.45 | A | 1H NMR (400 MHz, CDCl3) δ 8.01- | 1 |
| | | | | 7.94 (m, 2H), 7.57 (s, 1H), 7.54-7.46 | |
| | | | | (m, 1H), 7.22-7.12 (m, 2H), 6.09- | |
| | | | | 6.01 (m, 1H, 3.88 (t, J = 6.6 Hz, 2H), | |
| | | | | 3.38 (d, J = 6.4 Hz, 2H), 3.05 (t, J = | |
| | | | | 6.6 Hz, 2H), 1.65 (s, 6H), 1.57 (s, 9H). | |
| 454 | 3.33 | 438.42 | A | 1H NMR (400 MHz, CDCl3) δ 8.01- | 1 |
| | | | | 7.93 (m, 2H), 7.57 (s, 1H), 7.46 (s, | |
| | | | | 1H), 7.24-7.13 (m, 2H), 5.68 (s, 1H), | |
| | | | | 4.06 (t, J = 5.8 Hz, 2H), 3.59 (td, J = | |
| | | | | 5.8, 2.9 Hz, 2H), 2.82 (s, 2H), 1.72 (s, | |
| | | | | 6H), 1.55 (s, 9H). | |
| 455 | 4.23 | 550.43 | A | 1H NMR (400 MHz, DMSO-d6) δ 8.58 | 22A, 11 |
| | | | | (d, J = 4.8 Hz, 1H), 7.40 (s, 1H), 7.13 | |
| | | | | (s, 1H), 7.10 (d, J = 4.8 Hz, 1H), 4.11- | |
| | | | | 3.93 (m, 4H), 3.73 (t, J = 5.7 Hz, 2H), | |
| | | | | 2.75-2.65 (m, 1H), 1.81-1.58 (m, 4H), | |
| | | | | 1.50 (s, 6H), 1.43 (s, 9H), 1.42-1.41 | |
| | | | | (m, 2H), 1.30 (td, J = 13.1, 4.2 Hz, 2H), | |
| | | | | 0.94 (s, 3H), 0.92 (s, 4H). | |
| 456 | 3.95 | 567.23 | A | 1H NMR (400 MHz, DMSO-d6) δ | 22A, 11 |
| | | | | 12.67 (s, 1H), 8.65 (d, J = 1.3 Hz, 1H), | |
| | | | | 8.22 (s, 1H), 8.15 (dd, J = 11.0, 2.1 Hz, | |
| | | | | 1H), 8.06-7.93 (m, 1H), 7.81 (s, 1H), | |
| | | | | 7.69 (t, J = 8.1 Hz, 1H), 7.54 (s, 1H), | |
| | | | | 4.12 (dd, J = 7.1, 4.2 Hz, 2H), 4.01 (s, | |
| | | | | 2H), 3.85-3.59 (m, 2H), 1.52 (s, 6H), | |
| | | | | 1.51 (s, 9H). | |
| 457 | 4.16 | 550.24 | A | 1H NMR (400 MHz, DMSO-d6) δ | 22A, 11 |
| | | | | 13.49 (s, 1H), 8.62 (d, J = 4.8 Hz, 1H), | |
| | | | | 8.16 (d, J = 8.6 Hz, 2H), 7.78 (s, 1H), | |
| | | | | 7.57 (d, J = 8.6 Hz, 2H), 7.55 (s, 1H), | |
| | | | | 7.14 (d, J = 4.8 Hz, 1H), 4.08 (dd, J = | |
| | | | | 12.6, 6.3 Hz, 4H), 3.78 (t, J = 5.7 Hz, | |
| | | | | 2H), 1.54 (s, 15H). | |
| 458 | 4.31 | 561.28 | A | 1H NMR (400 MHz, DMSO-d6) δ | 3, 11 |
| | | | | 13.30 (s, 1H), 8.13-8.07 (m, 1H), 8.07- | |
| | | | | 8.00 (m, 1H), 7.90 (dd, J = 7.3, 1.0 | |
| | | | | Hz, 1H), 7.55 (s, 1H), 7.19 (s, 1H), | |
| | | | | 4.33-4.23 (m, 2H), 4.10-4.01 (m, | |
| | | | | 2H), 2.78-2.64 (m, 1H), 1.85 (s, 6H), | |
| | | | | 1.82-1.65 (m, 4H), 1.53-1.42 (m, | |
| | | | | 11H), 1.40-1.28 (m, 2H), 0.97 (d, J = | |
| | | | | 9.9 Hz, 6H). | |
| 459 | 4.35 | 580.43 | A | | 3, 11 |
| 460 | 4.19 | 562.46 | A | | 3, 11 |
| 461 | 4.43 | 566.46 | A | 1H NMR (400 MHz, DMSO-d6) δ | 22A, 11 |
| | | | | 13.48 (s, 1H), 8.62 (d, J = 4.8 Hz, 1H), | |
| | | | | 8.18 (dd, J = 11.0, 2.1 Hz, 1H), 8.09- | |
| | | | | 7.96 (m, 1H), 7.84 (s, 1H), 7.72 (t, J = | |
| | | | | 8.1 Hz, 1H), 7.56 (s, 1H), 7.14 (d, J = | |
| | | | | 4.8 Hz, 1H), 4.08 (dd, J = 12.0, 6.0 Hz, | |
| | | | | 4H), 3.87-3.68 (m, 2H), 1.55 (s, 15H). | |
| 462 | 4.32 | 564.29 | A | 1H NMR (400 MHz, DMSO-d6) δ | 22A, 11 |
| | | | | 12.69 (s, 1H), 8.77 (d, J = 2.7 Hz, 1H), | |
| | | | | 8.16 (d, J = 8.6 Hz, 2H), 7.78 (s, 1H), | |
| | | | | 7.56 (d, J = 8.6 Hz, 2H), 7.54 (s, 1H), | |
| | | | | 4.53-4.03 (m, 4H), 3.81 (s, 2H), 2.61 (s, | |
| | | | | 3H), 1.54 (s, 16H). | |
| 463 | 4.03 | 548.44 | A | 1H NMR (400 MHz, DMSO-d6) δ | 22A, 11 |
| | | | | 13.41-12.89 (m, 1H), 8.39 (s, 2H), 8.16 | |
| | | | | (d, J = 8.6 Hz, 2H), 7.79 (s, 1H), 7.67- | |
| | | | | 7.39 (m, 3H), 4.13 (dd, J = 7.4, 4.0 Hz, | |
| | | | | 2H), 3.99 (s, 2H), 3.81-3.66 (m, 2H), | |
| | | | | 1.55 (s, 6H), 1.54 (s, 9H). | |
| 464 | 4.03 | 548.44 | A | 1H NMR (400 MHz, DMSO-d6) δ | 22A, 11 |
| | | | | 13.23 (s, 1H), 8.39 (s, 1H), 8.38 (s, | |
| | | | | 1H), 7.45 (s, 1H), 7.16 (s, 1H), 4.10 | |
| | | | | (t, J = 5.6 Hz, 2H), 3.98 (s, 2H), 3.78- | |
| | | | | 3.69 (m, 2H), 2.81-2.65 (m, 1H), | |
| | | | | 1.86-1.61 (m, 4H), 1.54 (s, 6H), 1.47 | |
| | | | | (s, 9H), 1.46-1.43 (m, 2H), 1.34 (td, | |
| | | | | J = 13.3, 4.1 Hz, 2H), 0.98 (s, 3H), | |
| | | | | 0.95 (s, 3H). | |
| 465 | 4.56 | 562.63 | A | 1H NMR (400 MHz, DMSO-d6) δ | 22A, 11 |
| | | | | 12.62 (brs, 1H), 8.73 (d, J = 2.7 Hz, | |
| | | | | 1H), 7.41 (s, 1H), 7.15 (s, 1H), 4.02 | |
| | | | | (d, J = 5.2 Hz, 4H), 3.76 (s, 2H), 2.69 | |
| | | | | (tt, J = 11.8, 4.0 Hz, 1H), 2.57 (s, 3H), | |
| | | | | 1.84-1.61 (m, 4H), 1.49 (s, 6H), 1.46- | |
| | | | | 1.45 (m, 2H), 1.44 (s, 9H), 1.30 (td, | |
| | | | | J = 13.0, 3.8 Hz, 2H), 0.94 (s, 3H), | |
| | | | | 0.91 (s, 3H). | |
| 466 | 4.5 | 580.46 | A | 1H NMR (400 MHz, DMSO-d6) δ | 22A, 11 |
| | | | | 12.69 (s, 1H), 8.77 (d, J = 2.6 Hz, 1H), | |
| | | | | 8.18 (dd, J = 11.0, 2.1 Hz, 1H), 8.08- | |
| | | | | 7.96 (m, 1H), 7.84 (s, 1H), 7.72 (t, J = | |
| | | | | 8.1 Hz, 1H), 7.55 (s, 1H), 4.26-3.99 (m, | |
| | | | | 4H), 3.87-3.67 (m, 2H), 2.61 (s, 3H), | |
| | | | | 1.54 (s, 9H), 1.54 (s, 6H). | |
| 467 | 3.71 | 575.46 | A | 1H NMR (400 MHz, DMSO-d6) δ 8.16 | 3 |
| | | | | (d, J = 8.5 Hz, 2H), 7.78 (d, J = 1.0 Hz, | |
| | | | | 1H), 7.63-7.45 (m, 3H), 7.20 (dt, J = | |
| | | | | 8.8, 5.8 Hz, 1H), 3.99 (t, J = 5.4 Hz, | |
| | | | | 1H), 3.97-3.88 (m, 3H), 3.74-3.69 (m, | |
| | | | | 1H), 3.68 (s, 1H), 3.63 (s, 1H), 3.54 | |
| | | | | (t, J = 5.8 Hz, 1H), 3.10-2.98 (m, 2H), | |
| | | | | 1.53 (s, 9H), 1.53 (s, 3H), 1.50 (s, 3H), | |
| | | | | 1.23 (td, J = 7.3, 2.7 Hz, 3H). | |
| 468 | 3.83 | 593.43 | A | 1H NMR (400 MHz, DMSO-d6) δ 8.14 | 3 |
| | | | | (dd, J = 11.0, 2.0 Hz, 1H), 8.01 (dd, J = | |
| | | | | 8.5, 2.0 Hz, 1H), 7.80 (d, J = 1.1 Hz, | |
| | | | | 1H), 7.69 (td, J = 8.2, 1.1 Hz, 1H), 7.52 | |
| | | | | (d, J = 9.8 Hz, 1H), 7.17 (dt, J = 8.6, 5.7 | |
| | | | | Hz, 1H), 3.96 (t, J = 5.6 Hz, 1H), 3.93- | |
| | | | | 3.82 (m, 3H), 3.67 (t, J = 5.8 Hz, 1H), | |
| | | | | 3.65 (s, 1H), 3.60 (s, 1H), 3.51 (dd, J = | |
| | | | | 7.0, 4.6 Hz, 1H), 3.08-2.91 (m, 2H), | |
| | | | | 1.50 (s, 9H), 1.50 (s, 3H), 1.47 (s, 3H), | |
| | | | | 1.20 (td, J = 7.3, 2.6 Hz, 3H). | |
| 469 | 4.14 | 566.47 | A | 1H NMR (400 MHz, DMSO-d6) δ 8.36 | 22A, 11 |
| | | | | (s, 2H), 8.16-8.10 (m, 1H), 8.01 (dd, | |
| | | | | J = 8.3, 2.1 Hz, 1H), 7.82 (d, J = 5.7 | |
| | | | | Hz, 1H), 7.69 (t, J = 8.1 Hz, 1H), 7.54 | |
| | | | | (s, 0H), 4.15-4.03 (m, 2H), 3.96 (s, | |
| | | | | 2H), 3.78-3.66 (m, 2H), 1.58-1.43 (m, | |
| | | | | 15H). | |
| 470 | 4.19 | 566.47 | A | 1H NMR (400 MHz, DMSO-d6) δ 8.25 | 22A, 11 |
| | | | | (d, J = 2.3 Hz, 1H), 8.14 (dd, J = 11.0, | |
| | | | | 2.0 Hz, 1H), 8.03-7.98 (m, 1H), 7.94 | |
| | | | | (d, J = 2.3 Hz, 1H), 7.80 (s, 1H), 7.69 | |
| | | | | (td, J = 8.1, 2.1 Hz, 1H), 7.52 (s, 1H), | |
| | | | | 4.12-3.97 (m, 2H), 3.71 (s, 2H), 3.57- | |
| | | | | 3.45 (m, 2H), 1.58-1.41 (m, 15H). | |
| 471 | 3.5 | 548.44 | A | 1H NMR (400 MHz, DMSO-d6) δ 8.16 | 22A, 11 |
| | | | | (d, J = 8.6 Hz, 2H), 7.90 (d, J = 9.5 Hz, | |
| | | | | 1H), 7.79 (s, 1H), 7.58 (s, 1H), 7.56 (d, | |
| | | | | J = 2.2 Hz, 2H), 7.19 (d, J = 9.6 Hz, | |
| | | | | 1H), 4.17 (t, J = 5.6 Hz, 2H), 4.09 (s, | |
| | | | | 2H), 3.79 (t, J = 5.7 Hz, 2H), 1.56 (s, | |
| | | | | 6H), 1.55 (s, 9H). | |
| 472 | 3.73 | 591.47 | A | | 3 |
| 473 | 3.73 | 591.47 | A | | 3 |
| 474 | 3.59 | 573.46 | A | | 3 |
| 475 | 3.6 | 573.46 | A | | 3 |
| 476 | 3.9 | 569.66 | A | 1H NMR (400 MHz, DMSO-d6) δ 8.27- | 22A, 11 |
| | | | | 8.13 (m, 1H), 8.06-7.93 (m, 1H), | |
| | | | | 7.83 (s, 1H), 7.71 (t, J = 8.2 Hz, 1H), | |
| | | | | 7.56 (s, 1H), 4.02 (dd, J = 7.0, 3.9 Hz, | |
| | | | | 2H), 3.77-3.67 (m, 4H), 2.27 (s, 3H), | |
| | | | | 1.55 (s, 6H), 1.53 (s, 9H). | |
| 477 | 3.37 | 547.53 | A | 1H NMR (400 MHz, DMSO-d6) δ 8.39 | 22B |
| | | | | (d, J = 1.5 Hz, 1H), 8.36 (d, J = 3.0 Hz, | |
| | | | | 1H), 8.21-8.11 (m, 2H), 7.79 (s, 1H), | |
| | | | | 7.60-7.54 (m, 3H), 7.52 (dd, J = 3.1, | |
| | | | | 1.7 Hz, 1H), 4.09 (t, J = 5.2 Hz, 2H), | |
| | | | | 3.69 (s, 2H), 3.56 (t, J = 5.4 Hz, 2H), | |
| | | | | 1.59-1.48 (m, 15H). | |
| 478 | 3.89 | 551.45 | A | 1H NMR (400 MHz, DMSO-d6) δ 8.18- | 22B, 11 |
| | | | | 8.13 (m, 2H), 7.78 (d, J = 0.8 Hz, 1H), | |
| | | | | 7.58 (d, J = 2.0 Hz, 1H), 7.55 (d, J = | |
| | | | | 2.3 Hz, 2H), 4.00 (dt, J = 22.5, 5.6 Hz, | |
| | | | | 2H), 3.77-3.65 (m, 3H), 3.62 (s, 1H), | |
| | | | | 2.26 (s, 2H), 2.07 (s, 3H), 1.60-1.47 | |
| | | | | (m, 15H). | |
| 479 | 4.13 | 549.31 | A | 1H NMR (400 MHz, DMSO-d6) δ 8.16 | 22A, 11 |
| | | | | (d, J = 8.6 Hz, 2H), 7.78 (s, 1H), 7.69 | |
| | | | | (dd, J = 8.5, 7.3 Hz, 1H), 7.61-7.41 | |
| | | | | (m, 3H), 7.27 (d, J = 7.2 Hz, 1H), 6.84 | |
| | | | | (d, J = 8.5 Hz, 1H), 4.10 (t, J = 5.5 Hz, | |
| | | | | 2H), 4.00 (s, 2H), 3.65 (t, J = 5.6 Hz, | |
| | | | | 2H), 1.54 (s, 9H), 1.54 (s, 6H). | |
| 480 | 4.11 | 547.48 | A | 1H NMR (400 MHz, DMSO-d6) δ 8.65 | 22B, 11 |
| | | | | (dd, J = 2.3, 0.7 Hz, 1H), 8.20-8.12 (m, | |
| | | | | 2H), 7.98 (dd, J = 9.0, 2.4 Hz, 1H), | |
| | | | | 7.79 (s, 1H), 7.59-7.57 (m, 1H), 7.57- | |
| | | | | 7.54 (m, 2H), 6.69 (d, J = 9.0 Hz, 1H), | |
| | | | | 4.12 (t, J = 5.6 Hz, 2H), 4.02 (s, 2H), | |
| | | | | 3.68 (t, J = 5.6 Hz, 2H), 1.60-1.46 (m, | |
| | | | | 15H). | |
| 481 | 3.94 | 537.5 | A | 1H NMR (400 MHz, DMSO-d6) δ 8.19- | 22B, 11 |
| | | | | 8.12 (m, 3H), 7.78 (s, 1H), 7.59-7.53 | |
| | | | | (m, 3H), 4.04-3.96 (m, 2H), 3.72-3.60 | |
| | | | | (m, 4H), 1.58-1.48 (m, 15H). | |
| I-482 | 4.18 | 595.3 | A | 1H NMR (400 MHz, Methanol-d4) δ | 1, 11 |
| | | | | 7.93 (dd, J = 10.7, 2.1 Hz, 1H), 7.83 | |
| | | | | (m, 1H), 7.75 (s, 1H), 7.63-7.53 (m, | |
| | | | | 2H), 7.42 (s, 1H), 6.92 (d, J = 9.3 Hz, | |
| | | | | 1H), 4.14 (dd, J = 6.4, 4.9 Hz, 2H), | |
| | | | | 3.91 (s, 2H), 3.84 (s, 3H), 3.69 (dd, J = | |
| | | | | 6.3, 5.0 Hz, 2H), 1.62 (s, 6H), 1.59 (s, | |
| | | | | 9H). | |
| I-483 | 4.23 | 518.38 | A | 1H NMR (400 MHz, DMSO-d6) δ 8.85 | 22A |
| | | | | (s, 1H), 8.84 (s, 1H), 8.16 (d, J = 8.7 | |
| | | | | Hz, 2H), 7.79 (s, 1H), 7.65 (s, 1H), | |
| | | | | 7.56 (d, J = 8.7 Hz, 2H), 7.40 (t, J = | |
| | | | | 4.8 Hz, 1H), 4.25-4.14 (m, 2H), 4.05 | |
| | | | | (t, J = 4.9 Hz, 2H), 1.83 (s, 6H), 1.53 | |
| | | | | (s, 9H). | |
| I-484 | 3.9 | 558.2 | A | 1H NMR (400 MHz, DMSO-d6) δ | Ex. 88 |
| | | | | 12.35 (s, 1H), 8.11 (dd, J = 11.0, 2.1 | |
| | | | | Hz, 1H), 8.01-7.94 (m, 1H), 7.78 (s, | |
| | | | | 1H), 7.65 (t, J = 8.2 Hz, 1H), 7.53 (s, | |
| | | | | 1H), 3.75 (t, J = 4.9 Hz, 2H), 3.57 (s, | |
| | | | | 2H), 3.50 (t, J = 4.8 Hz, 2H), 1.65 (s, | |
| | | | | 6H), 1.46 (s, 9H), 1.06 (s, 6H). | |
| I-485 | 3.82 | 579.48 | A | 1H NMR (400 MHz, DMSO-d6) δ 8.17 | Ex. 89 |
| | | | | (dd, J = 11.0, 2.1 Hz, 1H), 8.09-8.00 | |
| | | | | (m, 2H), 7.83 (s, 1H), 7.72 (t, J = 8.1 | |
| | | | | Hz, 1H), 7.54 (s, 1H), 6.66 (d, J = 5.1 | |
| | | | | Hz, 1H), 3.99 (t, J = 5.4 Hz, 2H), 3.63 | |
| | | | | (s, 2H), 3.59-3.51 (m, 2H), 2.25 (s, | |
| | | | | 3H), 1.54 (s, 9H), 1.53 (s, 6H). | |
| I-486 | 3.52 | 455.55 | A | 1H NMR (400 MHz, DMSO-d6) δ 7.53- | 3 |
| | | | | 7.42 (m, 1H), 7.34 (s, 1H), 7.15 (s, | |
| | | | | 1H), 3.96-3.88 (m, 2H), 3.39-3.34 | |
| | | | | (m, 2H), 2.77-2.64 (m, 3H), 1.85- | |
| | | | | 1.63 (m, 4H), 1.54 (s, 6H), 1.45 (s, | |
| | | | | 11H), 1.40-1.27 (m, 2H), 0.98 (s, 3H), | |
| | | | | 0.95 (s, 3H). | |
| I-487 | 4.54 | 579.1 | A | 1H NMR (400 MHz, DMSO-d6) δ | 22B, 11 |
| | | | | 12.44 (s, 1H), 8.14 (dd, J = 11.0, 2.1 | |
| | | | | Hz, 1H), 8.06-7.95 (m, 1H), 7.80 (s, | |
| | | | | 1H), 7.69 (t, J = 8.1 Hz, 1H), 7.52 (s, | |
| | | | | 1H), 7.50 (d, J = 8.8 Hz, 1H), 6.77 (d, | |
| | | | | J = 8.7 Hz, 1H), 4.04 (t, J = 5.7 Hz, | |
| | | | | 2H), 3.89 (s, 2H), 3.60 (t, J = 5.6 Hz, | |
| | | | | 2H), 2.30 (s, 3H), 1.51 (s, 9H), 1.50 | |
| | | | | (s, 6H). | |
| I-488 | 4.46 | 575.6 | A | 1H NMR (400 MHz, DMSO-d6) δ | 22B, 11 |
| | | | | 13.07 (s, 1H), 7.87 (d, J = 8.4 Hz, 1H), | |
| | | | | 7.79 (d, J = 8.5 Hz, 1H), 7.50 (s, 1H), | |
| | | | | 7.15 (s, 1H), 4.18 (dd, J = 6.7, 3.3 Hz, | |
| | | | | 2H), 3.99 (t, J = 4.8 Hz, 2H), 2.68 (tt, | |
| | | | | J = 11.7, 4.1 Hz, 1H), 2.42 (s, 3H), | |
| | | | | 1.80 (s, 6H), 1.77-1.62 (m, 4H), 1.46 | |
| | | | | (s, 2H), 1.43 (s, 9H), 1.31 (td, J = 13.0, | |
| | | | | 3.9 Hz, 2H), 0.95 (s, 3H), 0.92 (s, 3H). | |
| I-489 | 3.77 | 454.75 | A | 1H NMR (400 MHz, DMSO-d6) δ 7.91- | 1 |
| | | | | 7.83 (m, 1H), 7.32 (s, 1H), 7.16 (s, | |
| | | | | 1H), 3.78-3.69 (m, 2H), 3.30-3.18 | |
| | | | | (m, 2H), 2.82-2.74 (m, 2H), 2.76- | |
| | | | | 2.64 (m, 1H), 1.86-1.60 (m, 4H), 1.49 | |
| | | | | (s, 2H), 1.46 (s, 15H), 1.40-1.27 (m, | |
| | | | | 2H), 0.98 (s, 3H), 0.95 (s, 3H). | |
| I-490 | 3.2 | 566.2 | A | 1H NMR (400 MHz, Methanol-d4) δ | 22A, 11 |
| | | | | 8.63 (d, J = 0.9 Hz, 1H), 7.93 (dd, J = | |
| | | | | 10.7, 2.1 Hz, 1H), 7.83 (dd, J = 8.1, | |
| | | | | 2.0 Hz, 1H), 7.76 (s, 1H), 7.59 (q, J = | |
| | | | | 8.2 Hz, 1H), 7.45 (s, 1H), 7.26 (s, 1H), | |
| | | | | 4.11 (m, 6H), 1.66 (s, 6H), 1.59 (s, 9H). | |
| I-491 | 3.19 | 565.66 | A | 1H NMR (400 MHz, DMSO-d6) δ | 22A |
| | | | | 12.94 (s, 1H), 8.41 (s, 1H), 8.18 (d, J = | |
| | | | | 6.0 Hz, 1H), 8.16-8.03 (m, 1H), 7.97 | |
| | | | | (dd, J = 8.5, 2.1 Hz, 1H), 7.77 (s, 1H), | |
| | | | | 7.65 (t, J = 8.1 Hz, 1H), 7.50 (s, 1H), | |
| | | | | 6.81 (d, J = 6.1 Hz, 1H), 4.00 (d, J = | |
| | | | | 5.5 Hz, 2H), 3.45 (s, 4H), 1.47 (s, 6H), | |
| | | | | 1.47 (s, 9H). | |
| I-492 | 4.66 | 579.29 | A | 1H NMR (400 MHz, DMSO-d6) δ | 22A |
| | | | | 12.91 (s, 1H), 8.17 (dd, J = 11.0, 2.1 | |
| | | | | Hz, 1H), 8.10 (d, J = 2.3 Hz, 1H), 8.04 | |
| | | | | (dd, J = 8.5, 2.0 Hz, 1H), 7.83 (s, 1H), | |
| | | | | 7.81-7.75 (m, 1H), 7.72 (t, J = 8.1 Hz, | |
| | | | | 1H), 7.55 (s, 1H), 4.01 (t, J = 5.2 Hz, | |
| | | | | 2H), 3.59 (s, 2H), 3.50 (t, J = 5.6 Hz, | |
| | | | | 2H), 2.20 (s, 3H), 1.53 (s, 9H), 1.53 | |
| | | | | (s, 6H). | |
| I-493 | 4.43 | 580.46 | A | 1H NMR (400 MHz, DMSO-d6) δ | 3 |
| | | | | 11.70 (s, 1H), 8.16, 8.17 (app d, J = | |
| | | | | 8.7 Hz, 2H), 7.79, 7.78 (s, 1H), 7.58 (d, | |
| | | | | J = 2.0 Hz, 1H), 7.58, 7.56 (app d, J = | |
| | | | | 8.7 Hz, 2H), 7.54, 7.52 (s, 1H), 3.97 (t, | |
| | | | | J = 5.6 Hz, 1H), 3.91 (t, J = 5.8 Hz, | |
| | | | | 1H), 3.71 (t, J = 5.7 Hz, 1H), 3.65 (s, | |
| | | | | 1H), 3.63 (s, 1H), 3.49 (t, J = 5.8 Hz, | |
| | | | | 1H), 2.75 (s, 1H), 2.69 (s, 1H), 2.06 | |
| | | | | (dt, J = 11.2, 5.6 Hz, 2H), 1.62 (d, J = | |
| | | | | 7.0 Hz, 3H), 1.54 (s, 6H), 1.54 (d, J = | |
| | | | | 1.5 Hz, 9H), 1.48 (s, 3H). | |
| I-494 | 4.23 | 579.25 | A | 1H NMR (400 MHz, Methanol-d4) δ | 1, 11 |
| | | | | 7.93 (dd, J = 10.7, 2.1 Hz, 1H), 7.83 | |
| | | | | (ddd, J = 8.5, 2.1, 0.8 Hz, 1H), 7.75 (s, | |
| | | | | 1H), 7.58 (dd, J = 8.4, 7.6 Hz, 1H), | |
| | | | | 7.42 (s, 1H), 7.29 (d, J = 1.0 Hz, 1H), | |
| | | | | 6.74 (t, J = 1.1 Hz, 1H), 4.26-4.15 (m, | |
| | | | | 2H), 4.05 (s, 2H), 3.79-3.67 (m, 2H), | |
| | | | | 2.36 (s, 3H), 1.62 (s, 6H), 1.59 (s, 9H). | |
| I-495 | 4.66 | 575.6 | A | 1H NMR (400 MHz, DMSO-d6) δ 8.20- | 22B, 11 |
| | | | | 8.08 (m, 2H), 7.94-7.81 (m, 2H), 7.80 | |
| | | | | (s, 1H), 7.65 (s, 1H), 7.60-7.51 (m, 2H), | |
| | | | | 4.23 (d, J = 5.4 Hz, 2H), 4.06 (s, 2H), | |
| | | | | 1.85 (s, 6H), 1.54 (s, 9H). | |
| I-496 | 4.8 | 561.74 | A | 1H NMR (400 MHz, DMSO-d6) δ | 22A, 11 |
| | | | | 12.25 (s, 1H), 7.95 (d, J = 8.9 Hz, 1H), | |
| | | | | 7.40 (s, 1H), 7.13 (s, 1H), 6.49 (d, J = | |
| | | | | 8.9 Hz, 1H), 4.03 (t, J = 5.7 Hz, 2H), | |
| | | | | 3.94 (s, 2H), 3.63 (t, J = 5.5 Hz, 2H), | |
| | | | | 2.75-2.63 (m, 1H), 1.83-1.63 (m, 3H), | |
| | | | | 1.48 (s, 6H), 1.46-1.45 (m, 2H), 1.44 | |
| | | | | (s, 9H), 1.37-1.25 (m, 2H), 0.95 (s, 3H), | |
| | | | | 0.92 (s, 3H). | |
| I-497 | 3.67 | 614.25 | A | 1H NMR (400 MHz, Methanol-d4) δ | Ex. 92 |
| | | | | 7.92 (dt, J = 10.7, 2.3 Hz, 1H), 7.86- | |
| | | | | 7.78 (m, 1H), 7.74 (d, J = 4.2 Hz, 1H), | |
| | | | | 7.57 (ddd, J = 8.5, 7.6, 2.3 Hz, 1H), | |
| | | | | 7.41 (d, J = 5.5 Hz, 1H), 4.04 (dt, J = | |
| | | | | 19.1, 5.8 Hz, 2H), 3.89-3.55 (m, 8H), | |
| | | | | 2.83 (s, 1H), 2.76 (s, 1H), 2.23-2.06 | |
| | | | | (m, 2H), 1.73-1.53 (m, 17H). | |
| I-498 | 4.39 | 595.25 | A | 1H NMR (400 MHz, DMSO-d6) δ | 1 |
| | | | | 11.83 (s, 1H), 8.14 (dd, J = 11.0, 2.1 | |
| | | | | Hz, 1H), 8.05-7.98 (m, 1H), 7.94 (d, | |
| | | | | J = 8.6 Hz, 1H), 7.81 (s, 1H), 7.69 (t, | |
| | | | | J = 8.1 Hz, 1H), 7.53 (s, 1H), 6.23 (s, | |
| | | | | 1H), 4.16-4.02 (m, 2H), 3.97 m, 2H), | |
| | | | | 3.85 (s, 3H), 3.67 (s, 2H), 1.51 (s, 15H). | |
| I-499 | 3.55 | 596.75 | A | 1H NMR (400 MHz, Methanol-d4) δ | 3 |
| | | | | 8.07-7.91 (m, 2H), 7.72 (d, J = 3.9 | |
| | | | | Hz, 1H), 7.55-7.46 (m, 2H), 7.40 (d, | |
| | | | | J = 4.9 Hz, 1H), 4.04 (dt, J = 18.7, 5.8 | |
| | | | | Hz, 2H), 3.81 (t, J = 5.6 Hz, 1H), 3.72 | |
| | | | | (d, J = 8.5 Hz, 6H), 3.62 (t, J = 5.8 Hz, | |
| | | | | 1H), 2.63 (s, 1H), 2.76 (s, 1H), 2.13 (d, | |
| | | | | J = 13.9 Hz, 2H), 1.80-1.51 (m, 17H). | |
| I-500 | 4.2 | 598.65 | A | 1H NMR (400 MHz. Methanol-d4) δ | 3, 11 |
| | | | | 7.93 (dd, J = 10.7, 2.0 Hz, 1H), 7.83 | |
| | | | | (dd, J = 8.5, 2.0 Hz, 1H), 7.75 (d, J = | |
| | | | | 2.1 Hz, 1H), 7.58 (t, J = 8.0 Hz, 1H), | |
| | | | | 7.41 (d, J = 3.2 Hz, 1H), 4.07-3.90 | |
| | | | | (m, 2H), 3.89-3.65 (m, 2H), 3.54- | |
| | | | | 3.40 (m, 1H), 2.59-2.48 (m, 1H), 2.29 | |
| | | | | (t, J = 12.7 Hz, 1H), 2.00-1.75 (m, | |
| | | | | 3H), 1.74-1.23 (m, 20H). | |
| I-501 | 5.15 | 584.42 | A | 1H NMR (400 MHz, DMSO-d6) δ 8.15 | 22A, 11 |
| | | | | (dd, J = 11.0, 2.1 Hz, 1H), 8.01 (dd, J = | |
| | | | | 8.5, 2.1 Hz, 1H), 7.81 (s, 1H), 7.69 (t, | |
| | | | | J = 8.1 Hz, 1H), 7.61-7.54 (m, 1H), 7.53 | |
| | | | | (s, 1H), 7.38 (dd, J = 8.1, 2.8 Hz, 1H), | |
| | | | | 3.98 (d, J = 5.7 Hz, 2H), 3.80 (s, 4H), | |
| | | | | 1.55 (s, 6H), 1.51 (s, 9H). | |
| I-502 | 5.05 | 566.41 | A | 1H NMR (400 MHz, DMSO-d6) δ | 22A, 11 |
| | | | | 12.77 (s, 1H), 8.13 (d, J = 8.6 Hz, 2H), | |
| | | | | 7.75 (s, 1H), 7.62-7.57 (m, 1H), 7.55- | |
| | | | | 7.50 (m, 3H), 7.40 (dd, J = 8.0, 2.8 Hz, | |
| | | | | 1H), 3.99 (t, J = 5.6 Hz, 2H), 3.81 (s, | |
| | | | | 4H), 1.55 (s, 6H), 1.51 (s, 9H). | |
| I-503 | 5.21 | 566.08 | A | 1H NMR (400 MHz, DMSO-d6) δ 7.59 | 22A, 11 |
| | | | | (dd, J = 13.4, 8.0 Hz, 1H), 7.43 (s, 1H), | |
| | | | | 7.41 (dd, J = 8.1, 2.8 Hz, 1H), 7.16 (s, | |
| | | | | 1H), 3.98 (t, J = 5.4 Hz, 2H), 3.81 (s, | |
| | | | | 4H), 2.76-2.65 (m, 1H), 1.88-1.65 | |
| | | | | (m, 4H), 1.56 (s, 6H), 1.49 (d, J = 3.5 | |
| | | | | Hz, 2H), 1.46 (s, 9H), 1.39-1.29 (m, | |
| | | | | 2H), 0.98 (s, 3H), 0.95 (s, 3H). | |
| I-504 | 4.42 | 554.46 | A | 1H NMR (400 MHz, DMSO-d6) δ | 3 |
| | | | | 12.87 (s, 1H), 8.14 (d, J = 11.4 Hz, | |
| | | | | 1H), 8.00 (d, J = 8.1 Hz, 1H), 7.80 (d, | |
| | | | | J = 5.2 Hz, 1H), 7.68 (t, J = 8.1 Hz, | |
| | | | | 1H), 7.50 (d, J = 6.7 Hz, 1H), 6.34 (s, | |
| | | | | 1H), 4.24 (s, 1H), 4.12-3.86 (m, 3H), | |
| | | | | 3.77 (s, 1H), 3.64 (t, J = 5.7 Hz, 1H), | |
| | | | | 2.23 (d, J = 4.6 Hz, 3H), 1.51 (d, J = | |
| | | | | 2.5 Hz, 9H), 1.48 (s, 3H), 1.45 (s, 3H). | |
| I-505 | 3.69 | 507.45 | A | 1H NMR (400 MHz, DMSO-d6) δ 8.21 | 22A |
| | | | | (s, 1H), 8.16 (d, J = 8.7 Hz, 2H), 7.79 | |
| | | | | (s, 1H), 7.58 (s, 1H), 7.57 (d, J = 8.6 | |
| | | | | Hz, 2H), 3.82 (t, J = 5.2 Hz, 2H), 3.53 | |
| | | | | (s, 3H), 3.43-3.37 (m, 2H), 3.16 (s, | |
| | | | | 2H), 1.58 (s, 6H), 1.54 (s, 9H). | |
| I-506 | 4.08 | 598.3 | K | 1H NMR (400 MHz, Methanol-d4) δ | 3, 11 |
| | | | | 7.93 (m, 1H), 7.83 (m, 1H), 7.75 (d, | |
| | | | | J = 3.9 Hz, 1H), 7.58 (m, 1H), 7.41 (d, | |
| | | | | J = 6.1 Hz, 1H), 4.08 (m, 3H), 3.89- | |
| | | | | 3.73 (m, 2H), 3.70-3.47 (m, 1H), 2.99- | |
| | | | | 2.62 (m, 2H), 2.15 (s, 1H), 1.84 (m, | |
| | | | | 3H), 1.69-1.53 (m, 15H), 1.37 (m, 4H). | |
| I-507 | 4.1 | 627.25 | A | 1H NMR (400 MHz, DMSO-d6) δ | Ex. 92 |
| | | | | 10.94 (s, 1H, br), 8.21-8.13 (m, 1H), | |
| | | | | 8.07-8.00 (m, 1H), 7.95-7.88 (m, 2H), | |
| | | | | 7.83 (s, 1H), 7.76-7.55 (m, 4H), 7.52 | |
| | | | | (s, 1H), 3.93-3.88 (m, 2H), 3.70-3.45 | |
| | | | | (m, 4H), 1.53 (s, 9H), 1.46 (s, 6H). | |
| I-508 | 4.14 | 524.15 | A | 1H NMR (400 MHz, DMSO-d6) δ 8.21- | 12 |
| | | | | 8.13 (m, 1H), 8.07-8.00 (m, 1H), 7.84 | |
| | | | | (s, 1H), 7.76-7.67 (m, 1H), 7.56 (s, 1H), | |
| | | | | 3.87-3.79 (m, 2H), 3.46-3.38 (m, 2H), | |
| | | | | 3.24 (s, 2H), 2.98 (s, 3H), 1.57-1.48 | |
| | | | | (m, 15H). | |
| I-509 | 3.47 | 704.73 | A | 1H NMR (400 MHz, DMSO-d6) δ 7.42, | Ex. 95 |
| | | | | 7.38 (s, 1H), 7.15, 7.14 (s, 1H), 5.45 (d, | |
| | | | | J = 12.2 Hz, 2H), 3.96-3.81 (m, 2H), | |
| | | | | 3.80-3.72 (m, 1H), 3.68 (s, 1H), 3.61 (s, | |
| | | | | 1H), 3.51-3.37 (m, 1H), 2.77-2.55 (m, | |
| | | | | 1H), 2.21-2.07 (m, 2H), 1.89-1.16 (m, | |
| | | | | 30H), 1.12, 1.13 (s, 3H), 0.97 (s, 3H), | |
| | | | | 0.95 (s, 3H). | |
| I-510 | 4.04 | 580.1 | A | 1H NMR (400 MHz, Methanol-d4) δ | 1, 11 |
| | | | | 8.05-7.89 (m, 2H), 7.84 (m, 1Hz, 1H), | |
| | | | | 7.76 (s, 1H), 7.59 (m, 1H), 7.42 (s, 1H), | |
| | | | | 5.94 (d, J = 8.9 Hz, 1H), 4.15 (t, J = 5.7 | |
| | | | | Hz, 2H), 3.99 (s, 2H), 3.71 (t, J = 5.7 | |
| | | | | Hz, 2H), 1.61 (s, 6H), 1.59 (s, 9H). | |
| I-511 | 3.98 | 580.25 | A | 1H NMR (400 MHz, Methanol-d4) δ | 3, 11 |
| | | | | 8.08-7.94 (m, 2H), 7.72 (d, J = 3.8 Hz, | |
| | | | | 1H), 7.55-7.46 (m, 2H), 7.40 (d, J = | |
| | | | | 5.8 Hz, 1H), 4.20-4.00 (m, 3H), 3.91- | |
| | | | | 3.76 (m, 2H), 3.72-3.55 (m, 1H), | |
| | | | | 2.99-2.66 (m, 2H), 2.15 (m, 1H), | |
| | | | | 1.84 (m, 3H), 1.68-1.57 (m, 15H), | |
| | | | | 1.37 (m, 4H). | |
| I-512 | 3.84 | 584.3 | A | 1H NMR (400 MHz, Methanol-d4) δ | 3, 11 |
| | | | | 7.93 (m, 1H), 7.83 (m, 1H), 7.75 (d, | |
| | | | | J = 2.6 Hz, 1H), 7.58 (m, 1H), 7.41 | |
| | | | | (d, J = 3.0 Hz, 1H), 4.14-3.99 (m, | |
| | | | | 2H), 3.85 (t, J = 5.9 Hz, 1H), 3.77 | |
| | | | | (d, J = 6.1 Hz, 2H), 3.69-3.61 (m, | |
| | | | | 1H), 3.24-3.03 (m, 1H), 2.83 (m, | |
| | | | | 1H), 2.21 (m, 1H), 2.14-1.86 (m, | |
| | | | | 5H), 1.67-1.57 (m, 15H). | |
| I-513 | 3.71 | 558.7 | A | 1H NMR (400 MHz, Methanol-d4) δ | 3, 11 |
| | | | | 7.93 (m, 1H), 7.83 (m, 1H), 7.75 (d, | |
| | | | | J = 2.1 Hz, 1H), 7.62-7.56 (m, 1H), | |
| | | | | 7.42 (d, J = 3.3 Hz, 1H), 4.15-3.99 | |
| | | | | (m, 3H), 3.94-3.81 (m, 2H), 3.74- | |
| | | | | 3.60 (m, 1H), 3.23-3.09 (m, 1H), | |
| | | | | 2.79 (m, 1H), 2.39 (m, 1H), 1.71- | |
| | | | | 1.57 (m, 15H), 1.14 (dd, J = 7.0, 4.2 | |
| | | | | Hz, 3H). | |
| I-514 | 3.71 | 558.6 | A | 1H NMR (400 MHz, Methanol-d4) δ | 3, 11 |
| | | | | 7.93 (m, 1H), 7.83 (m, 1H), 7.75 (s, | |
| | | | | 1H), 7.58 (m, 1H), 7.42 (s, 1H), 4.17- | |
| | | | | 4.00 (m, 3H), 3.89-3.79 (m, 2H), | |
| | | | | 3.71-3.58 (m, 1H), 3.22-3.07 (m, 1H), | |
| | | | | 2.79 (m, 1H), 2.40 (m, 1H), 1.70-1.56 | |
| | | | | (m, 15H), 1.14 (dd, J = 7.0, 4.2 Hz, 3H). | |
| I-515 | 4.84 | 633.53 | A | 1H NMR (400 MHz, DMSO-d6) δ 8.34- | 22A |
| | | | | 8.26 (m, 1H), 8.17 (dd, J = 11.0, 2.1 | |
| | | | | Hz, 1H), 8.04 (dd, J = 8.5, 2.0 Hz, 1H), | |
| | | | | 7.98 (d, J = 2.1 Hz, 1H), 7.83 (s, 1H), | |
| | | | | 7.72 (t, J = 8.1 Hz, 1H), 7.62 (d, J = 1.8 | |
| | | | | Hz, 2H), 7.55 (s, 1H), 4.02 (t, J = 5.1 | |
| | | | | Hz, 2H), 3.50 (d, J = 6.2 Hz, 4H), 1.56 | |
| | | | | (s, 6H), 1.53 (s, 9H). | |
| I-516 | 5.45 | 641.65 | A | 1H NMR (400 MHz, DMSO-d6) δ 8.17 | 22A |
| | | | | (dd, J = 11.0, 2.1 Hz, 1H), 8.16-8.11 | |
| | | | | (m, 2H), 8.09-7.99 (m, 2H), 7.83 (s, | |
| | | | | 1H), 7.71 (t, J = 8.1 Hz, 1H), 7.56 (s, | |
| | | | | 1H), 7.53-7.41 (m, 3H), 7.38 (d, J = | |
| | | | | 7.9 Hz, 1H), 4.08 (dd, J = 7.4, 3.7 Hz, | |
| | | | | 2H), 3.77 (s, 2H), 3.72-3.63 (m, 2H), | |
| | | | | 1.57 (s, 6H), 1.54 (s, 9H). | |
| I-517 | 4.93 | 583.35 | A | 1H NMR (400 MHz, DMSO-d6) δ 8.11 | 22A |
| | | | | (dt, J = 11.0, 2.0 Hz, 1H), 8.04 (s, 1H), | |
| | | | | 7.97 (dt, J = 8.5, 2.1 Hz, 1H), 7.77 (d, | |
| | | | | J = 1.9 Hz, 1H), 7.65 (td, J = 8.1, 2.3 | |
| | | | | Hz, 1H), 7.49 (d, J = 4.8 Hz, 1H), 6..91, | |
| | | | | 6.72, 6.51 (s, 1H), 4.00 (t, J = 5.7 Hz, | |
| | | | | 1H), 3.79 (d, J = 10.0 Hz, 2H), 3.53 (t, | |
| | | | | J = 5.7 Hz, 1H), 3.20 (s, 2H, partially | |
| | | | | obscured by H2O), 1.51-1.41 (m, 15H). | |
| I-518 | 5.05 | 583.3 | A | 1H NMR (400 MHz, DMSO-d6) δ | 22A |
| | | | | 13.27 (s, 1H), 8.33 (d, J = 3.0 Hz, 1H), | |
| | | | | 8.18 (dd, J = 11.0, 2.1 Hz, 1H), 8.05 | |
| | | | | (dd, J = 8.4, 2.0 Hz, 1H), 7.87 (dd, J = | |
| | | | | 8.6, 3.1 Hz, 1H), 7.84 (s, 1H), 7.72 (t, | |
| | | | | J = 8.2 Hz, 1H), 7.56 (s, 1H), 4.03 (t, | |
| | | | | J = 5.4 Hz, 2H), 3.60 (s, 2H), 3.55-3.45 | |
| | | | | (m, 2H), 1.55 (s, 6H), 1.54 (s, 9H). [2], | |
| | | | | 1H NMR (400 MHz, DMSO-d6) d | |
| | | | | 13.36 (s, 1H), 8.24 (d, J = 3.0 Hz, 1H), | |
| | | | | 8.17 (dd, J = 11.0, 2.1 Hz, 1H), 8.04 | |
| | | | | (dd, J = 8.4, 2.0 Hz, 1H), 7.83 (s, 1H), | |
| | | | | 7.79-7.74 (m, 1H), 7.71 (t, J = 8.2 Hz, | |
| | | | | 1H), 7.54 (s, 1H), 4.07-3.93 (m, 2H), | |
| | | | | 3.59 (s, 2H), 3.54-3.46 (m, 2H), 1.53 | |
| | | | | (d, J = 1.5 Hz, 15H). | |
| I-519 | | | A | 1H NMR (400 MHz, DMSO-d6) δ 8.47 | 22A |
| | | | | (s, 1H), 8.38 (s, 1H), 8.11 (dd, J = 11.0, | |
| | | | | 2.1 Hz, 1H), 7.97 (dd, J = 8.5, 2.1 Hz, | |
| | | | | 1H), 7.76 (s, 1H), 7.65 (t, J = 8.1 Hz, | |
| | | | | 1H), 7.48 (s, 1H), 6.52 (s, 1H), 4.00 | |
| | | | | (dd, J = 7.3, 3.9 Hz, 2H), 3.76 (s, 2H), | |
| | | | | 3.56 (t, J = 5.6 Hz, 2H), 1.46 (s, 16H). | |
| I-520 | 5.02 | 580.55 | A | 1H NMR (400 MHz, DMSO-d6) δ | 22A |
| | | | | 13.43 (s, 1H), 8.38 (s, 1H), 8.14 (dd, | |
| | | | | J = 11.0, 2.0 Hz, 1H), 8.01 (dd, J = 8.5, | |
| | | | | 2.0 Hz, 1H), 7.80 (s, 1H, 7.68 (t, J = | |
| | | | | 8.1 Hz, 1H), 7.51 (s, 1H), 4.03 (d, J = | |
| | | | | 6.5 Hz, 2H), 3.96 (s, 2H), 3.70 (t, J = | |
| | | | | 5.7 Hz, 2H), 2.16 (s, 3H), 1.51 (s, 9H), | |
| | | | | 1.50 (s, 6H). | |
| I-521 | 4.91 | 562.67 | A | 1H NMR (400 MHz, DMSO-d6) δ | 22A |
| | | | | 13.44 (s, 1H), 8.38 (d, J = 0.7 Hz, 1H), | |
| | | | | 8.13 (d, J = 8.7 Hz, 2H), 7.75 (s, 1H), | |
| | | | | 7.53 (d, J = 8.6 Hz, 2H), 7.50 (s, 1H), | |
| | | | | 4.03 (t, J = 5.7 Hz, 2H), 3.96 (s, 2H), | |
| | | | | 3.70 (t, J = 5.7 Hz, 2H), 2.26-2.04 (m, | |
| | | | | 3H), 1.51 (s, 9H), 1.50 (s, 6H). | |
| I-522 | 3.81 | 488.45 | A | 1H NMR (400 MHz, Chloroform-d) δ | 14 |
| | | | | 7.84 (dd, J = 10.3, 2.1 Hz, 1H), 7.78- | |
| | | | | 7.70 (m, 1H), 7.58 (s, 1H), 7.55-7.42 | |
| | | | | (m, 2H), 4.11-4.03 (m, 2H), 3.67- | |
| | | | | 3.59 (m, 2H), 3.04 (s, 3H), 2.87 (s, | |
| | | | | 2H), 1.59-1.53 (m, 15H). | |
| I-523 | 4.11 | 535.04 | A | 1H NMR (400 MHz, DMSO-d6) δ | 3 |
| | | | | 12.90 (s, 1H), 7.41 (d, J = 5.6 Hz, 1H), | |
| | | | | 7.16 (d, J = 5.2 Hz, 1H), 6.39 (d, J = | |
| | | | | 13.2 Hz, 1H), 4.24 (s, 1H), 4.07 (s, | |
| | | | | 1H), 3.99 (t, J = 5.8 Hz, 1H), 3.91 (s, | |
| | | | | 1H), 3.78 (s, 1H), 3.65 (t, J = 5.8 Hz, | |
| | | | | 1H), 2.80-2.64 (m, 1H), 2.25 (d, J = | |
| | | | | 5.2 Hz, 3H), 1.88-1.64 (m, 4H), 1.53 | |
| | | | | (s, 3H), 1.50-1.47 (m, 2H), 1.46 (s, | |
| | | | | 9H), 1.43 (s, 3H), 1.34 (dt, J = 14.1, | |
| | | | | 7.1 Hz, 2H), 0.98 (s, 3H), 0.95 (s, 3H). | |
| I-524 | 4.33 | 580.55 | A | 1H NMR (400 MHz, Methanol-d4) δ | 22A, 11 |
| | | | | 7.98-7.91 (m, 2H), 7.84 (dt, J = 8.5, | |
| | | | | 1.3 Hz, 1H), 7.76 (s, 1H), 7.59 (t, | |
| | | | | J = 8.0 Hz, 1H), 7.44 (s, 1H), 4.22 | |
| | | | | (t, J = 5.6 Hz, 2H), 4.09 (s, 2H), 3.85 | |
| | | | | (t, J = 5.7 Hz, 2H), 2.70 (s, 3H), 1.64 | |
| | | | | (s, 6H), 1.59 (s, 9H). | |
| I-525 | 4.83 | 563.09 | A | 1H NMR (400 MHz, DMSO-d6) δ | 22A |
| | | | | 13.42 (s, 1H), 8.38 (s, 1H), 7.39 (s, | |
| | | | | 1H), 7.13 (s, 1H), 3.99 (t, J = 5.7 Hz, | |
| | | | | 2H), 3.94 (s, 2H), 3.68 (t, J = 5.7 Hz, | |
| | | | | 2H), 2.68 (tt, J = 11.8, 4.0 Hz, 1H), | |
| | | | | 2.16 (s, 3H), 1.82-1.62 (m, 4H), 1.48 | |
| | | | | (s, 6H), 1.47-1.44 (m, 2H), 1.43 (s, | |
| | | | | 9H), 1.36-1.22 (m, 2H), 0.95 (s, 3H), | |
| | | | | 0.92 (s, 3H). | |
| I-526 | 3.77 | 486.55 | A | | 14 |
| I-527 | 3.68 | 567.1 | A | 1H NMR (400 MHz, DMSO-d6) δ | Ex. 92 |
| | | | | 10.48 (s, 1H), 8.17 (dd, J = 11.0, 2.1 | |
| | | | | Hz, 1H), 8.07-8.00 (m, 1H), 7.83 (s, | |
| | | | | 1H), 7.76-7.67 (m, 1H), 7.53 (s, 1H), | |
| | | | | 3.98-3.90 (m, 2H), 3.64-3.53 (m, | |
| | | | | 4H), 3.26 (s, 3H), 1.53 (s, 9H), 1.51 | |
| | | | | (s, 6H). | |
| I-528 | 3.23 | 580.41 | A | 1H NMR (400 MHz, Chloroform-d) δ | 22A |
| | | | | 7.89 (s, 1H), 7.83 (dd, J = 10.3, 2.0 Hz, | |
| | | | | 1H), 7.75-7.69 (m, 1H), 7.58 (s, 1H), | |
| | | | | 7.55-7.48 (m, 2H), 4.03 (t, J = 5.2 Hz, | |
| | | | | 2H), 3.62 (t, J = 5.2 Hz, 2H), 3.49 (s, | |
| | | | | 2H), 2.79 (s, 3H), 1.71 (s, 6H), 1.55 | |
| | | | | (s, 9H). | |
| I-529 | 4.29 | 589.75 | A | 1H NMR (400 MHz, DMSO-d6) δ 8.75- | 1 |
| | | | | 8.70 (m, 1H), 8.18-8.12 (m, 1H), 8.11- | |
| | | | | 8.06 (m, 1H), 8.03-7.97 (m, 1H), 7.81 | |
| | | | | (d, J = 0.9 Hz, 1H), 7.73-7.64 (m, 1H), | |
| | | | | 7.53 (d, J = 1.0 Hz, 1H), 6.82 (d, J = | |
| | | | | 8.9 Hz, 1H), 4.14-4.07 (m, 2H), 3.99 | |
| | | | | (s, 2H), 3.67 (t, J = 5.7 Hz, 2H), 1.52 | |
| | | | | (s, 15H). | |
| I-530 | 3.24 | 507.24 | A | 1H NMR (400 MHz, DMSO-d6) δ 8.21 | 22A |
| | | | | (s, 1H), 7.47 (s, 1H), 7.17 (s, 1H), 3.85- | |
| | | | | 3.71 (m, 2H), 3.53 (s, 3H), 3.43-3.32 | |
| | | | | (m, 2H), 3.14 (s, 2H), 2.83-2.65 (m, | |
| | | | | 1H), 1.88-1.65 (m, 4H), 1.57 (s, 6H), | |
| | | | | 1.52-1.48 (m, 2H), 1.47 (s, 9H), 1.34 | |
| | | | | (td, J = 13.3, 3.9 Hz, 2H), 0.98 (s, 3H), | |
| | | | | 0.96 (s, 4H). | |
| I-531 | 3.92 | 568.97 | A | 1H NMR (400 MHz, DMSO-d6) δ 8.73 | 1 |
| | | | | 8.73 (d, J = 2.4 Hz, 1H), 8.13 (d, J = | |
| | | | | 8.6 Hz, 2H), 8.09 (dd, J = 9.0, 2.4 Hz, | |
| | | | | 1H), 7.75 (s, 1H), 7.55 (s, 1H), 7.52 (s, | |
| | | | | 2H), 6.83 (d, J = 9.0 Hz, 1H), 4.11 (t, | |
| | | | | J = 5.6 Hz, 2H), 3.99 (s, 2H), 3.67 (t, | |
| | | | | J = 5.7 Hz, 2H), 1.52 (s, 9H). | |
| I-532 | 4.65 | 603.52 | A | 1H NMR (400 MHz, DMSO-d6) δ 8.15 | 1 |
| | | | | (dd, J = 11.0, 2.1 Hz, 1H), 8.05-7.96 | |
| | | | | (m, 1H), 7.81 (s, 1H), 7.73-7.63 (m, | |
| | | | | 1H), 7.64-7.57 (m, 1H), 7.53 (s, 1H), | |
| | | | | 6.73 (d, J = 8.7 Hz, 1H), 4.30-3.84 | |
| | | | | (m, 4H), 3.63 (t, J = 5.6 Hz, 2H), 2.53 | |
| | | | | (s, 3H), 1.52 (d, J = 2.5 Hz, 15H). | |
| I-533 | 4.72 | 586.01 | A | 1H NMR (400 MHz, DMSO-d6) δ 7.62- | 1 |
| | | | | 7.55 (m, 1H), 7.40 (s, 1H), 7.13 (s, 1H), | |
| | | | | 6.70 (d, J = 8.6 Hz, 1H), 4.03 (d, J = 7.0 | |
| | | | | Hz, 4H), 3.60 (t, J = 5.5 Hz, 2H), 2.68 | |
| | | | | (tt, J = 11.6, 4.0 Hz, 1H), 2.50 (s, 3H), | |
| | | | | 1.82-1.70 (m, 2H), 1.73-1.62 (m, 2H), | |
| | | | | 1.50 (s, 6H), 1.44 (s, 11H), 1.31 (td, | |
| | | | | J = 13.0, 3.9 Hz, 2H), 0.95 (s, 3H), 0.92 | |
| | | | | (s, 3H). | |
| I-534 | 3.7 | 595.3 | A | 1H NMR (400 MHz, Methanol-d4) δ | 1, 11 |
| | | | | 8.57 (s, 1H), 7.93 (dd, J = 10.7, 2.1 Hz, | |
| | | | | 1H), 7.83 (ddd, J = 8.4, 2.0, 0.8 Hz, | |
| | | | | 1H), 7.76 (s, 1H), 7.65-7.52 (m, 1H), | |
| | | | | 7.44 (s, 1H), 6.10 (s, 1H), 4.21 (dd, J = | |
| | | | | 7.6, 3.8 Hz, 2H), 4.05 (s, 2H), 3.94 (s, | |
| | | | | 3H), 3.74 (t, J = 5.7 Hz, 2H), 1.63 (s, | |
| | | | | 6H), 1.59 (s, 9H). | |
| I-535 | 4.75 | 578.22 | A | 1H NMR (400 MHz, Methanol-d4) δ | 1, 11 |
| | | | | 8.08 (d, J = 8.7 Hz, 1H), 7.33 (s, 1H), | |
| | | | | 7.23 (s, 1H), 6.23 (d, J = 8.7 Hz, 1H), | |
| | | | | 4.15 (t, J = 5.7 Hz, 2H), 4.03 (s, 2H), | |
| | | | | 3.98 (s, 3H), 3.76 (t, J = 5.6 Hz, 2H), | |
| | | | | 2.74 (m, 1H), 1.91-1.70 (m, 4H), 1.57 | |
| | | | | (m, 17H), 1.41 (m, 2H), 1.04 (s, 3H), | |
| | | | | 0.97 (s, 3H). | |
| I-536 | 3.8 | 567.67 | A | 1H NMR (400 MHz, Methanol-d4) δ | 3, 11 |
| | | | | 8.04-7.90 (m, 2H), 7.71 (d, J = 2.6 | |
| | | | | Hz, 1H), 7.55-7.45 (m, 2H), 7.40 (d, | |
| | | | | J = 2.9 Hz, 1H), 4.16-4.00 (m, 2H), | |
| | | | | 3.86 (d, J = 5.3 Hz, 1H), 3.77 (d, J = | |
| | | | | 5.9 Hz, 2H), 3.65 (d, J = 11.0 Hz, | |
| | | | | 1H), 3.24-3.04 (m, 1H), 2.82 (tt, J = | |
| | | | | 10.4, 7.8 Hz, 1H), 2.21 (dtd, J = | |
| | | | | 13.2, 7.7, 5.8 Hz, 1H), 2.12-1.84 (m, | |
| | | | | 5H), 1.69-1.53 (m, 15H). | |
| I-537 | 4.56 | 585.64 | A | 1H NMR (400 MHz, DMSO-d6) δ 8.13 | 1 |
| | | | | (d, J = 8.6 Hz, 2H), 7.75 (s, 1H), 7.58 | |
| | | | | (d, J = 8.7 Hz, 1H), 7.54 (d, J = 8.7 Hz, | |
| | | | | 2H), 7.52 (s, 1H), 6.71 (d, J = 8.7 Hz, | |
| | | | | 1H), 4.36-3.85 (m, 4H), 3.62 (t, J = | |
| | | | | 5.6 Hz, 2H), 2.49 (s, 3H), 1.54-1.49 | |
| | | | | (m, 15H). | |
| I-538 | 4.12 | 579.71 | A | 1H NMR (400 MHz, Methanol-d4) δ | 3, 11 |
| | | | | 8.18 (dd, J = 5.1, 0.7 Hz, 1H), 7.93 | |
| | | | | (dd, J = 10.7, 2.1 Hz, 1H), 7.83 (ddd, | |
| | | | | J = 8.4, 2.1, 0.8 Hz, 1H), 7.75 (s, 1H), | |
| | | | | 7.58 (dd, J = 8.5, 7.6 Hz, 1H), 7.44 (s, | |
| | | | | 1H), 7.28 (d, J = 5.1 Hz, 1H), 3.95 | |
| | | | | (dd, J = 6.3, 4.2 Hz, 2H), 3.50-3.40 | |
| | | | | (m, 2H), 3.25 (s, 2H), 2.48 (s, 3H), | |
| | | | | 1.67 (s, 6H), 1.58 (s, 9H). | |
| I-539 | 4.03 | 568.79 | | 1H NMR (400 MHz, DMSO-d6) δ 8.72 | 1 |
| | | | | (d, J = 2.4 Hz, 1H), 8.08 (dd, J = 9.0, | |
| | | | | 2.4 Hz, 1H), 7.41 (s, 1H), 7.13 (s, 1H), | |
| | | | | 6.82 (d, J = 9.0 Hz, 1H), 4.07 (t, J = | |
| | | | | 5.6 Hz, 2H), 3.97 (s, 2H), 3.65 (t, J = | |
| | | | | 5.6 Hz, 2H), 2.76-2.60 (m, 1H), 1.85- | |
| | | | | 1.58 (m, 4H), 1.50 (s, 6H), 1.44 (s, | |
| | | | | 11H), 1.31 (td, J = 13.1, 4.3 Hz, 2H), | |
| | | | | 0.95 (s, 3H), 0.92 (s, 3H). | |
| I-540 | 4.48 | 643.38 | A | 1H NMR (400 MHz, DMSO-d6) δ | |
| | | | | 11.21 (s, 1H), 8.18 (dd, J = 11.0, | |
| | | | | 2.1 Hz, 1H), 8.08-8.01 (m, 1H), 7.84 | |
| | | | | (s, 1H), 7.80-7.68 (m, 2H), 7.56 (s, 1H), | |
| | | | | 7.33 (d, J = 7.2 Hz, 1H), 6.97 (d, J = | |
| | | | | 8.6 Hz, 1H), 4.11 (t, J = 5.6 Hz, 2H), | |
| | | | | 4.01 (s, 2H), 3.72 (d, J = 5.6 Hz, 2H), | |
| | | | | 3.35 (s, 3H), 1.59-1.53 (m, 15H). | |
| I-541 | 3.81 | 581.2 | A | 1H NMR (400 MHz, DMSO-d6) δ | Ex. 91 |
| | | | | 10.02 (s, 1H), 8.17 (dd, J = 11.0, 2.0 | |
| | | | | Hz, 1H), 8.03 (dd, J = 8.5, 2.0 Hz, 1H), | |
| | | | | 7.83 (s, 1H), 7.76-7.67 (m, 1H), 7.52 (s, | |
| | | | | 1H), 6.58 (d, J = 7.4 Hz, 1H), 3.84 (s, | |
| | | | | 2H), 3.32-3.27 (m, 1H), 3.23 (s, 3H), | |
| | | | | 2.14-1.93 (m, 1H), 1.83- 1.74 (m, 1H), | |
| | | | | 1.71-1.50 (m, 14H), 1.47 (s, 3H). | |
| I-542 | 4.35 | 565.2 | A | | Ex. 91 |
| I-543 | 3.72 | 570.65 | A | 1H NMR (400 MHz, Methanol-d4) δ | 3, 11 |
| | | | | 7.92 (dd, J = 10.7, 2.1 Hz, 1H), 7.83 | |
| | | | | (ddd, J = 8.4, 2.1, 0.8 Hz, 1H), 7.75 | |
| | | | | (d, J = 1.1 Hz, 1H), 7.58 (ddd, J = 8.5, | |
| | | | | 7.6, 1.0 Hz, 1H), 7.40 (d, J = 0.6 Hz, | |
| | | | | 1H), 4.00 (dt, J = 8.7, 5.7 Hz, 2H), | |
| | | | | 3.85-3.41 (m, 6H), 2.55-2.42 (m, 1H), | |
| | | | | 2.38-2.00 (m, 3H), 1.65-1.53 (m, 15H). | |
| I-544 | 4.35 | 562.4 | A | 1H NMR (400 MHz, Methanol-d4) δ | |
| | | | | 7.94 (s, 1H), 7.33 (s, 1H), 7.23 (s, 1H), | |
| | | | | 4.19 (t, J = 5.7 Hz, 2H), 4.08 (s, 2H), | |
| | | | | 3.83 (t, J = 5.7 Hz, 2H), 2.70 (s, 4H), | |
| | | | | 2.04-1.69 (m, 4H), 1.63 (s, 6H), 1.53 | |
| | | | | (m, 11H), 1.41 (m, 2H), 1.04 (s, 3H), | |
| | | | | 0.97 (s, 3H). | |
| I-545 | 3.92 | 584.65 | A | 1H NMR (400 MHz, Methanol-d4) δ | 3, 11 |
| | | | | 7.93 (m, 1H), 7.83 (dd, J = 8.3, 2.0 Hz, | |
| | | | | 1H), 7.75 (d, J = 2.5 Hz, 1H), 7.58 (m, | |
| | | | | 1H), 7.44-7.33 (m, 1H), 4.14-3.55 (m, | |
| | | | | 6H), 3.48-3.38 (m, 1H), 3.25-2.90 | |
| | | | | (m, 1H), 2.22-1.73 (m, 5H), 1.68- | |
| | | | | 1.55 (m, 16H). | |
| I-546 | 4.17 | 564.63 | A | 1H NMR (400 MHz, DMSO-d6) δ 8.15 | 1 |
| | | | | (dd, J = 11.0, 2.1 Hz, 1H), 8.05-7.98 | |
| | | | | (m, 1H), 8.00 (s, 1H), 7.81 (s, 1H), | |
| | | | | 7.74-7.62 (m, 2H), 7.52 (s, 1H), 7.48 | |
| | | | | (s, 1H), 7.24 (d, J = 7.6 Hz, 1H), | |
| | | | | 6.76 (d, J = 8.5 Hz, 1H), 4.05 (t, J = | |
| | | | | 5.6 Hz, 2H), 4.01 (s, 2H), 3.62 (t, | |
| | | | | J = 5.5 Hz, 2H), 1.54-1.49 (m, 15H). | |
| I-547 | 4.3 | 612.65 | A | 1H NMR (400 MHz, Methanol-d4) δ | 3 |
| | | | | 7.93 (m, 1H), 7.83 (m, 1H), 7.75 (d, | |
| | | | | J = 4.1 Hz, 1H), 7.58 (m, 1H), 7.41 (m, | |
| | | | | 1H), 4.03 (m, 2H), 3.82 (t, J = 5.6 Hz, | |
| | | | | 1H), 3.72 (d, J = 8.6 Hz, 2H), 3.64- | |
| | | | | 3.53 (m, 1H), 2.76 (s, 1H), 2.70 (s, | |
| | | | | 1H), 1.99 (d, J = 12.1 Hz, 2H), 1.67- | |
| | | | | 1.38 (m, 23H). | |
| I-548 | 2.72 | 456.15 | A | 1H NMR (400 MHz, DMSO-d6) δ 8.17 | Ex. 96 |
| | | | | (dd, J = 11.0, 2.1 Hz, 1H), 8.07-8.00 | |
| | | | | (m, 1H), 7.84 (s, 1H), 7.77-7.68 (m, | |
| | | | | 1H), 7.53 (s, 1H), 7.26 (s, 2H, br), | |
| | | | | 3.90-3.79 (m, 1H), 3.47-3.17 (m, 2H), | |
| | | | | 2.12-1.98 (m, 1H), 1.87-1.77 (m, | |
| | | | | 1H), 1.71-1.49 (m, 14H), 1.45 (s, 3H). | |
| I-549 | 2.7 | 472.25 | A | | Cf. Ex. 96 |

N/A = not available
Note:
General procedures and LCMS methods are described under the “Scheme and Examples” section.

TABLE 2
Compound Biological Data

| Com- | SLIGKV | | | | |
|---|---|---|---|---|---|
| pound | IC50 | Trypsin IC50 | | Thrombin | UTP IC50 |
| number | (μM) | (μM) | Method* | IC50 (μM) | (μM) |
| I-1 | 0.068 | 0.064 | A | 19 | 20 |
| I-2 | 0.022 | 0.029 | A | >50 | >50 |
| I-3 | 0.71 | 0.81 | B | 26 | 18 |
| I-4 | 0.21 | 0.19 | A | 8.4 | 12 |
| I-5 | 0.12 | 0.16 | A | 11 | 8.8 |
| I-6 | 0.0032 | 0.0042 | A | >50 | 36 |
| I-7 | 0.023 | 0.020 | A | 3.1 | 2.7 |
| I-8 | 0.029 | 0.028 | A | 3.6 | 20 |
| I-9 | 0.090 | 0.083 | A | 9.6 | 4.5 |
| I-10 | 0.023 | 0.027 | A | 15 | 16 |
| I-11 | 0.0024 | 0.0035 | A | 1.4 | 59% @ 50 |
| I-12 | 0.0083 | 0.0086 | A | 8.7 | 5.8 |
| I-13 | 0.0030 | 0.0045 | A | 1 | 1.2 |
| I-14 | 0.0028 | 0.0030 | A | 57% @ 50 | 59% @ 50 |
| I-15 | 0.23 | 0.19 | A | 14 | 14 |
| I-16 | 0.0072 | 0.0068 | A | 6.9 | 61% @ 50 |
| I-17 | 0.059 | 0.067 | A | 14 | 15 |
| I-18 | 0.0014 | 0.0011 | A | 7.9 | 17 |
| I-19 | 0.0056 | 0.0044 | A | 10 | 5.7 |
| I-20 | 0.011 | 0.010 | A | 7.1 | 1.5 |
| I-21 | 0.0014 | 0.0026 | A | >50 | 59% @ 50 |
| I-22 | 0.0024 | 0.0020 | A | >50 | 64% @ 50 |
| I-23 | 0.080 | 0.071 | A | 7.8 | 11 |
| I-24 | 0.048 | 0.044 | A | 8.7 | 9.9 |
| I-25 | 0.014 | 0.018 | A | 4.3 | 2.7 |
| I-26 | 0.0061 | 0.0080 | A | 4.8 | 7.0 |
| I-27 | 0.019 | 0.033 | A | 6.0 | 7.4 |
| I-28 | 0.0042 | 0.0035 | A | 8.8 | 11 |
| I-29 | 0.023 | 0.032 | A | 19 | 16 |
| I-30 | 0.016 | 0.014 | A | 20 | 13 |
| I-31 | 17 | 21 | B | >50 | >50 |
| I-32 | 0.0059 | 0.0046 | A | 11 | 6.7 |
| I-33 | 0.094 | 0.064 | A | 3.6 | 5.8 |
| I-34 | 0.0046 | 0.0059 | A | >25 | 26 |
| I-35 | 0.034 | 0.049 | A | 14 | 11 |
| I-36 | 0.00097 | 0.0015 | A | 31 | >26 |
| I-37 | 0.057 | 0.067 | A | 21 | 17 |
| I-38 | 0.0035 | 0.0031 | A | >50 | 39 |
| I-39 | 0.014 | 0.020 | A | 21 | 23 |
| I-40 | 0.0050 | 0.0073 | A | 37 | 30 |
| I-41 | 0.0065 | 0.0085 | A | >50 | >50 |
| I-42 | 0.0012 | 0.0012 | A | >50 | >50 |
| I-43 | 0.0069 | 0.0082 | A | >50 | >50 |
| I-44 | 0.0029 | 0.0032 | A | >40 | 36 |
| I-45 | 0.0040 | 0.0041 | A | 20 | 4.7 |
| I-46 | 0.0079 | 0.0070 | A | 4.1 | 5.3 |
| I-47 | 0.029 | 0.078 | A | >50 | >50 |
| I-48 | 0.0051 | 0.0059 | A | 16 | 16 |
| I-49 | 0.0059 | 0.012 | A | 15 | 18 |
| I-50 | 0.0049 | 0.0051 | A | >50 | >50 |
| I-51 | 0.0058 | 0.0053 | A | 24 | >50 |
| I-52 | 0.0042 | 0.006 | A | >50 | >50 |
| I-53 | 0.053 | 0.074 | A | >50 | >50 |
| I-54 | 0.0045 | 0.0063 | A | 9.6 | 7.9 |
| I-55 | 0.0028 | 0.0046 | A | 29 | >33 |
| I-56 | 0.0160 | 0.031 | A | 9 | 5.2 |
| I-57 | 0.053 | 0.077 | A | 6.8 | 8.6 |
| I-58 | 0.00031 | 0.00064 | A | 19 | 30 |
| I-59 | 0.021 | 0.049 | A | 15 | 15 |
| I-60 | 0.048 | 0.067 | A | 14 | 13 |
| I-61 | 0.0048 | 0.011 | A | 17 | 13 |
| I-62 | 0.04 | 0.039 | A | 4.7 | 5.2 |
| I-63 | 0.062 | 0.11 | A | 18 | 19 |
| I-64 | 0.0045 | 0.0060 | A | 30 | 21 |
| I-65 | 0.010 | 0.016 | A | 2.7 | 2.8 |
| I-66 | 0.013 | 0.015 | A | >50 | >50 |
| I-67 | 0.0052 | 0.0074 | A | >50 | >50 |
| I-68 | 0.010 | 0.026 | A | 29 | 21 |
| I-69 | 0.032 | 0.052 | A | 37 | >32 |
| I-70 | 0.017 | 0.023 | A | 15 | 16 |
| I-71 | 0.0026 | 0.0047 | A | 35 | 36 |
| I-72 | 0.0023 | 0.0031 | A | 14 | 13 |
| I-73 | 0.0024 | 0.0042 | A | 8.5 | 17 |
| I-74 | 0.0053 | 0.0077 | A | 11 | 13 |
| I-75 | 0.024 | 0.14 | A | 31 | 23 |
| I-76 | 0.033 | 0.10 | A | >50 | 56% @ 50 |
| I-77 | 64% @ 50 | 23 | B | >50 | >50 |
| I-78 | 70% @ 50 | 65% @ 50 | B | >50 | >50 |
| I-79 | 1.7 | 60% @ 50 | B | 26% @ 50 | >50 |
| I-80 | 1.6 | 4.7 | B | 19 | 20 |
| I-81 | 0.022 | 66% @ 50 | B | >50 | 46% @ 50 |
| I-82 | 0.18 | 70% @ 50 | B | >50 | 47 |
| I-83 | 0.0045 | 0.0078 | A | 11 | 29 |
| I-84 | 0.0063 | 0.029 | A | 50 | >25 |
| I-85 | 0.030 | 0.25 | A | >50 | >50 |
| I-86 | 0.39 | 0.53 | B | >50 | 60% @ 50 |
| I-87 | 3.7 | 3.8 | B | 19 | 16 |
| I-88 | 0.16 | 0.34 | A | 17 | 17 |
| I-89 | 1.0 | 1.1 | B | 13 | 11 |
| I-90 | 0.0026 | 0.0072 | A | >50 | 42 |
| I-91 | 3.3 | 3.8 | B | 9.4 | 11 |
| I-92 | 4.7 | 6.2 | B | 7.9 | 7.4 |
| I-93 | 18 | 16 | B | 25 | 29 |
| I-94 | 1.2 | 0.62 | B | 6.7 | 74% @ 50 |
| I-95 | 10 | 8.9 | B | 15 | 14 |
| I-96 | 13 | 12 | B | 17 | 14 |
| I-97 | 4.1 | 5.1 | B | 18 | 15 |
| I-98 | 5.3 | 7.0 | B | 18 | 20 |
| I-99 | 0.062 | 0.43 | A | 3.9 | 63% @ 50 |
| I-100 | 9.1 | 11 | B | 16 | 13 |
| I-101 | 7.6 | 9.1 | B | 18 | 13 |
| I-102 | 4.7 | 5.2 | B | 16 | 5.4 |
| I-103 | 7.9 | 8.5 | B | 29 | 24 |
| I-104 | 0.73 | 0.83 | B | 2.6 | 64% @ 50 |
| I-105 | 8.7 | 7.7 | B | 12 | 15 |
| I-106 | 7.8 | 7.7 | B | 15 | 14 |
| I-107 | 0.034 | 0.028 | A | >50 | >50 |
| I-108 | 0.0022 | 0.0018 | A | >50 | >50 |
| I-109 | 2.3 | 3.4 | B | 12 | 11 |
| I-110 | 0.040 | 0.064 | A | 18 | 14 |
| I-111 | 0.0565 | 0.041 | A | 19 | 12 |
| I-112 | 0.013 | 0.027 | A | 13 | 11 |
| I-113 | 0.011 | 0.0020 | A | 17 | 12 |
| I-114 | 0.0022 | 0.0027 | A | 27 | 30 |
| I-115 | 0.046 | 0.092 | A | >50 | 66% @ 50 |
| I-116 | 0.35 | 0.99 | B | 13 | 6.9 |
| I-117 | 1.1 | 2.3 | B | 12 | 7.7 |
| I-118 | 0.6 | 0.78 | B | 9.8 | 6.1 |
| I-119 | 0.62 | 0.56 | B | 1.4 | 1.6 |
| I-120 | 0.36 | 1.3 | B | 13.0 | 13 |
| I-121 | 1.1 | 1.8 | B | 5.5 | 6.3 |
| I-122 | 1.0 | 0.33 | B | 1.9 | 1.6 |
| I-123 | 0.34 | 0.93 | B | 4.5 | 4.1 |
| I-124 | 0.94 | 1.9 | B | 24 | 23 |
| I-125 | 0.26 | 1.9 | B | 32 | 20 |
| I-126 | 0.11 | 0.067 | A | 13 | 22 |
| I-127 | 0.74 | 49% @ 50 | B | >50 | >50 |
| I-128 | 0.54 | 5.3 | B | >50 | 44 |
| I-129 | 0.25 | 0.27 | A | >50 | >50 |
| I-130 | 2.1 | 2.6 | B | >35 | 30 |
| I-131 | 0.066 | 0.068 | A | 19 | 12 |
| I-132 | 0.0064 | 0.0090 | A | >50 | >50 |
| I-133 | 5.9 | 7.5 | B | 20 | 20 |
| I-134 | 0.0006 | 0.0009 | A | >1 | >1 |
| I-135 | 0.0240 | 0.042 | A | >1 | >1 |
| I-136 | 0.0064 | 0.0074 | A | >1 | >1 |
| I-137 | 0.0078 | 0.0094 | A | 0.50 | 0.83 |
| I-138 | 0.0020 | 0.0022 | A | >1 | >1 |
| I-139 | 0.024 | 0.033 | A | >1 | >1 |
| I-140 | 0.0048 | 0.0035 | A | >1 | >1 |
| I-141 | 0.0032 | 0.0042 | A | >1 | >1 |
| I-142 | 0.0072 | 0.013 | A | >1 | >1 |
| I-143 | 0.0021 | 0.0022 | A | >1 | >1 |
| I-144 | 0.024 | 0.031 | A | >1 | >1 |
| I-145 | 0.040 | 0.042 | A | >1 | >1 |
| I-146 | 0.013 | 0.021 | A | >1 | >1 |
| I-147 | 0.020 | 0.034 | A | >1 | >1 |
| I-148 | 0.046 | 0.036 | A | >1 | >1 |
| I-149 | 0.0076 | 0.0096 | A | >1 | >1 |
| I-150 | 0.0011 | 0.0016 | A | >1 | >1 |
| I-151 | 0.0015 | 0.0027 | A | >1 | >1 |
| I-152 | 0.0081 | 0.0077 | A | >1 | >1 |
| I-153 | 0.0049 | 0.0030 | A | >1 | >1 |
| I-154 | 0.0047 | 0.0036 | A | >1 | >1 |
| I-155 | 0.014 | 0.028 | A | >1 | >1 |
| I-156 | 0.018 | 0.026 | A | >1 | >1 |
| I-157 | 0.0092 | 0.0079 | A | >1 | >1 |
| I-158 | 0.0031 | 0.0039 | A | >1 | >1 |
| I-159 | 0.0006 | 0.0011 | A | >1 | >1 |
| I-160 | 0.0016 | 0.0031 | A | >1 | >1 |
| I-161 | 0.016 | 0.025 | A | >1 | >1 |
| I-162 | 0.010 | 0.015 | A | >1 | >1 |
| I-163 | 0.0029 | 0.0036 | A | >1 | >1 |
| I-164 | 0.0029 | 0.0040 | A | >1 | >1 |
| I-165 | 0.016 | 0.019 | A | >1 | >1 |
| I-166 | 0.0013 | 0.0033 | A | >1 | >1 |
| I-167 | 0.0084 | 0.019 | A | >1 | >1 |
| I-168 | 0.0028 | 0.003 | A | >1 | >1 |
| I-169 | 0.0009 | 0.0024 | A | >1 | >1 |
| I-170 | 0.0060 | 0.0082 | A | >1 | >1 |
| I-171 | 0.0056 | 0.0066 | A | >1 | >1 |
| I-172 | 0.010 | 0.012 | A | >1 | >1 |
| I-173 | 0.0048 | 0.0075 | A | >1 | >1 |
| I-174 | 0.0019 | 0.0032 | A | >1 | >1 |
| I-175 | 0.016 | 0.016 | A | >1 | >1 |
| I-176 | 0.0048 | 0.0050 | A | >1 | >1 |
| I-177 | 0.0014 | 0.0020 | A | >1 | >1 |
| I-178 | 0.0007 | 0.0010 | A | >1 | >1 |
| I-179 | 0.0028 | 0.0055 | A | >1 | >1 |
| I-180 | 0.0011 | 0.0023 | A | >1 | >1 |
| I-181 | 0.0009 | 0.0016 | A | >1 | >1 |
| I-182 | 0.0011 | 0.0058 | A | >1 | >1 |
| I-183 | 0.0026 | 0.0091 | A | >1 | >1 |
| I-184 | 0.0057 | 0.012 | A | >1 | >1 |
| I-185 | 0.0012 | 0.0051 | A | >1 | >1 |
| I-186 | 0.0007 | 0.0010 | A | >1 | >1 |
| I-187 | 0.01 | 0.02 | A | >1 | >1 |
| I-188 | 0.003 | 0.006 | A | >1 | >1 |
| I-189 | 0.017 | 0.028 | A | >1 | >1 |
| I-190 | 0.011 | 0.013 | A | 0.52 | 0.76 |
| I-191 | 0.403 | 0.713 | A | >1 | >1 |
| I-192 | 0.014 | 0.013 | A | >1 | >1 |
| I-193 | 0.26 | 0.19 | A | >1 | >1 |
| I-194 | 0.009 | 0.022 | A | >1 | >1 |
| I-195 | 0.096 | 0.233 | A | >1 | >1 |
| I-196 | 0.0007 | 0.001 | A | >1 | >1 |
| I-197 | 0.048 | 0.076 | A | >1 | >1 |
| I-198 | 0.0001 | 0.0008 | A | >1 | >1 |
| I-199 | 0.017 | 0.027 | A | >1 | >1 |
| I-200 | 0.003 | 0.006 | A | >1 | >1 |
| I-201 | 0.004 | 0.007 | A | >1 | >1 |
| I-202 | 0.005 | 0.005 | A | >1 | >1 |
| I-203 | 0.005 | 0.01 | A | >1 | >1 |
| I-204 | 0.006 | 0.004 | A | >1 | >1 |
| I-205 | 0.0006 | 0.001 | A | >1 | >1 |
| I-206 | 0.004 | 0.004 | A | >1 | >1 |
| I-207 | 0.05 | 0.035 | A | >1 | >1 |
| I-208 | 0.0007 | 0.001 | A | >1 | >1 |
| I-209 | 0.22 | 0.37 | A | >1 | >1 |
| I-210 | 0.002 | 0.003 | A | >1.5 | >1.5 |
| I-211 | 0.007 | 0.017 | A | >2 | >2 |
| I-212 | 0.013 | 0.043 | A | >2 | >2 |
| I-213 | 0.008 | 0.053 | A | >2 | >2 |
| I-214 | 0.027 | 0.026 | A | >1 | >1 |
| I-215 | 0.096 | 0.30 | A | >1 | >1 |
| I-216 | 0.068 | 0.24 | A | >1 | >1 |
| I-217 | 0.013 | 0.022 | A | >2 | >2 |
| I-218 | 0.036 | 0.044 | A | >2 | >2 |
| I-219 | 0.039 | 0.05 | A | >2 | >2 |
| I-220 | 0.0067 | 0.013 | A | >2 | >2 |
| I-221 | 0.032 | 0.058 | A | >2 | >2 |
| I-222 | 0.275 | 0.73 | A | >2 | >2 |
| I-223 | 0.0138 | 0.039 | A | >2 | >2 |
| I-224 | 0.0023 | 0.0033 | A | >2 | >2 |
| I-225 | 0.013 | 0.024 | A | >2 | >2 |
| I-226 | 0.076 | 0.23 | A | >2 | >2 |
| I-227 | 0.0008 | 0.0012 | A | >2 | >2 |
| I-228 | 0.0009 | 0.0014 | A | >2 | >2 |
| I-229 | 0.024 | 0.028 | A | >2 | >2 |
| I-230 | 0.081 | 0.15 | A | >2 | >2 |
| I-231 | 0.084 | 0.16 | A | >2 | >2 |
| I-232 | 0.215 | 0.2 | A | >2 | >2 |
| I-233 | 0.0062 | 0.0081 | A | >2 | >2 |
| I-234 | 0.022 | 0.038 | A | >2 | >2 |
| I-235 | 0.0056 | 0.0094 | A | >2 | >2 |
| I-236 | 0.0035 | 0.005 | A | >2 | >2 |
| I-237 | 0.0018 | 0.0024 | A | >2 | >2 |
| I-238 | 0.018 | 0.028 | A | >2 | >2 |
| I-239 | 0.0061 | 0.0054 | A | >2 | >2 |
| I-240 | 0.0027 | 0.0018 | A | >2 | >2 |
| I-241 | 0.0017 | 0.0016 | A | >2 | >2 |
| I-242 | 0.019 | 0.024 | A | >2 | >2 |
| I-243 | 0.004 | 0.0074 | A | >2 | >2 |
| I-244 | 0.019 | 0.027 | A | >2 | >2 |
| I-245 | 0.0025 | 0.0048 | A | >2 | >2 |
| I-246 | 0.011 | 0.016 | A | >2 | >2 |
| I-247 | 0.012 | 0.020 | A | >2 | >2 |
| I-248 | 0.011 | 0.041 | A | >2 | >2 |
| I-249 | 0.004 | 0.011 | A | >2 | >2 |
| I-250 | 0.015 | 0.051 | A | >2 | >2 |
| I-251 | >2 | >2 | A | >2 | >2 |
| I-252 | 0.12 | 0.14 | A | >2 | >2 |
| I-253 | 0.0035 | 0.0095 | A | >2 | >2 |
| I-254 | 0.0085 | 0.020 | A | >2 | >2 |
| I-255 | 0.011 | 0.013 | A | >2 | >2 |
| I-256 | 0.022 | 0.031 | A | >2 | >2 |
| I-257 | 0.21 | 0.18 | A | >2 | >2 |
| I-258 | 0.023 | 0.017 | A | >2 | >2 |
| I-259 | 0.008 | 0.0079 | A | >2 | >2 |
| I-260 | 0.0033 | 0.0044 | A | >2 | >2 |
| I-261 | 0.045 | 0.031 | A | >2 | >2 |
| I-262 | 0.77 | 1.9 | A | >2 | >2 |
| I-263 | 0.0026 | 0.0039 | A | >2 | >2 |
| I-264 | 0.015 | 0.018 | A | >2 | >2 |
| I-265 | 0.0058 | 0.0055 | A | >2 | >2 |
| I-266 | 0.036 | 0.033 | A | >2 | >2 |
| I-267 | 0.033 | 0.025 | A | >2 | >2 |
| I-268 | 0.0049 | 0.0094 | A | >2 | >2 |
| I-269 | 0.057 | 0.041 | A | >2 | >2 |
| I-270 | 0.021 | 0.051 | A | >2 | >2 |
| I-271 | 0.0074 | 0.0078 | A | >2 | >2 |
| I-272 | 0.021 | 0.014 | A | >2 | >2 |
| I-273 | 0.026 | 0.014 | A | >2 | >2 |
| I-274 | 0.061 | 0.034 | A | >2 | >2 |
| I-275 | 0.016 | 0.020 | A | >2 | >2 |
| I-276 | 0.0012 | 0.0018 | A | >2 | >2 |
| I-277 | 0.0033 | 0.0033 | A | >2 | >2 |
| I-278 | 0.052 | 0.089 | A | >2 | >2 |
| I-279 | 0.0017 | 0.0017 | A | >2 | >2 |
| I-280 | 0.036 | 0.0525 | A | >2 | >2 |
| I-281 | 0.0036 | 0.0074 | A | 1.75 | >2 |
| I-282 | 0.0075 | 0.0083 | A | 1.6 | 1.8 |
| I-283 | 0.018 | 0.0615 | A | >2 | >2 |
| I-284 | 0.013 | 0.0145 | A | >2 | >2 |
| I-285 | 0.028 | 0.0235 | A | >2 | >2 |
| I-286 | 0.0014 | 0.0026 | A | >2 | >2 |
| I-287 | 0.0003 | 0.0005 | A | 1.8 | 1.625 |
| I-288 | | | | | |
| I-289 | 0.0001 | 0.0003 | A | >2 | >2 |
| I-290 | 0.0032 | 0.0061 | A | >2 | >2 |
| I-291 | 0.0007 | 0.0011 | A | >2 | >2 |
| I-292 | 0.0035 | 0.0071 | A | >2 | >2 |
| I-293 | 0.0054 | 0.0067 | A | >2 | >2 |
| I-294 | 0.0015 | 0.0036 | A | >2 | >2 |
| I-295 | 0.0073 | 0.022 | A | >2 | >2 |
| I-296 | 0.0007 | 0.0007 | A | >2 | >2 |
| I-297 | 0.0002 | 0.0002 | A | 1.4 | 1.2 |
| I-298 | 0.014 | 0.018 | A | 0.51 | 0.51 |
| I-299 | 0.0002 | 0.0002 | A | >2 | 1.7 |
| I-300 | 0.0016 | 0.0016 | A | >2 | >2 |
| I-301 | 0.003 | 0.004 | A | >2 | >2 |
| I-302 | 0.014 | 0.0195 | A | >2 | >2 |
| I-303 | 0.0004 | 0.0006 | A | 1.3 | 1.8 |
| I-304 | 0.002 | 0.0028 | A | >2 | >2 |
| I-305 | 0.0015 | 0.003 | A | >2 | >2 |
| I-306 | 0.0047 | 0.017 | A | 1.4 | 1.8 |
| I-307 | 0.124 | 0.21 | A | >2 | >2 |
| I-308 | 0.014 | 0.037 | A | >2 | >2 |
| I-309 | 0.032 | 0.04 | A | >2 | >2 |
| I-310 | 0.061 | 0.11 | A | >2 | >2 |
| I-311 | 0.081 | 0.083 | A | >2 | >2 |
| I-312 | 0.17 | 0.26 | A | >2 | >2 |
| I-313 | 0.075 | 0.31 | A | >2 | >2 |
| I-314 | 0.19 | 0.73 | A | >2 | >2 |
| I-315 | 0.0057 | 0.012 | A | >2 | >2 |
| I-316 | 0.0002 | 0.0002 | A | >2 | >2 |
| I-317 | 0.0082 | 0.01 | A | >2 | >2 |
| I-318 | 0.0028 | 0.0046 | A | >2 | >2 |
| I-319 | 0.0002 | 0.0002 | A | >2 | >2 |
| I-320 | 0.0001 | 0.0002 | A | >2 | >2 |
| I-321 | 0.0001 | 0.0001 | A | >2 | >2 |
| I-322 | 0.092 | 0.24 | A | >2 | >2 |
| I-323 | 0.012 | 0.018 | A | >2 | >2 |
| I-324 | 0.0002 | 0.0004 | A | >2 | >2 |
| I-325 | 0.02 | 0.02 | A | >2 | >2 |
| I-326 | 0.023 | 0.038 | A | >2 | >2 |
| I-327 | 0.010 | 0.012 | A | >2 | >2 |
| I-328 | 0.016 | 0.022 | A | >2 | >2 |
| I-329 | 0.012 | 0.013 | A | >2 | >2 |
| I-330 | 0.021 | 0.032 | A | >2 | >2 |
| I-331 | 0.0013 | 0.0028 | A | 1.8 | >2 |
| I-332 | 0.0004 | 0.0006 | A | 1.3 | 1.4 |
| I-333 | 0.0004 | 0.0007 | A | 1.8 | 2 |
| I-334 | 0.0014 | 0.0016 | A | >2 | >2 |
| I-335 | 0.091 | 0.0285 | A | >2 | >2 |
| I-336 | 0.0063 | 0.0096 | A | >2 | >2 |
| I-337 | 0.0004 | 0.0009 | A | >2 | >2 |
| I-338 | 0.0003 | 0.0005 | A | >2 | >2 |
| I-339 | 0.0013 | 0.0031 | A | >2 | >2 |
| I-340 | 0.0009 | 0.0017 | A | >2 | >2 |
| I-341 | 0.0016 | 0.0044 | A | >2 | 1.7 |
| I-342 | 0.094 | 0.09 | A | >2 | >2 |
| I-343 | 0.0002 | 0.0004 | A | 1.7 | 2.1 |
| I-344 | 0.0001 | 0.0002 | A | >2 | >2 |
| I-345 | 0.0003 | 0.0007 | A | >2 | >2 |
| I-346 | 0.0016 | 0.0041 | A | 2.1 | 1 |
| I-347 | 0.29 | 0.50 | A | >2 | >2 |
| I-348 | 0.067 | 0.038 | A | >2 | >2 |
| I-349 | 0.08 | 0.075 | A | >2 | >2 |
| I-350 | 0.00008 | 0.0003 | A | >2 | >2 |
| I-351 | 0.0051 | 0.009 | A | >2 | >2 |
| I-352 | 0.0021 | 0.0031 | A | >2 | 2 |
| I-353 | 0.038 | 0.034 | A | >2 | >2 |
| I-354 | 0.0003 | 0.0004 | A | >2 | >2 |
| I-355 | 0.014 | 0.016 | A | >2 | >2 |
| I-356 | 0.012 | 0.0141 | A | >2 | >2 |
| I-357 | 0.0083 | 0.010 | A | >2 | >2 |
| I-358 | 0.0034 | 0.0064 | A | >2 | >2 |
| I-359 | 0.037 | 0.043 | A | >2 | >2 |
| I-360 | 0.0024 | 0.0037 | A | >2 | >2 |
| I-361 | 0.0028 | 0.0046 | A | >2 | >2 |
| I-362 | 0.0007 | 0.001 | A | >1.1 | >1.1 |
| I-363 | 0.0006 | 0.0009 | A | >2 | >2 |
| I-364 | 0.0012 | 0.0012 | A | >2 | 1.7 |
| I-365 | 0.0002 | 0.0003 | A | >2 | >2 |
| I-366 | 0.0008 | 0.0011 | A | >2 | >2 |
| I-367 | 0.0002 | 0.0004 | A | >1.55 | >1.55 |
| I-368 | 0.012 | 0.0095 | A | >2 | >2 |
| I-369 | 0.012 | 0.019 | A | >2 | >2 |
| I-370 | 0.017 | 0.017 | A | >2 | >2 |
| I-371 | 0.0052 | 0.0052 | A | >2 | >2 |
| I-372 | 0.0002 | 0.0003 | A | >2 | >2 |
| I-373 | 0.0006 | 0.0008 | A | >1.1 | >1.1 |
| I-374 | 0.0036 | 0.0041 | A | >2 | >2 |
| I-375 | 0.0003 | 0.0007 | A | >2 | >2 |
| I-376 | 0.28 | 0.29 | A | >2 | >2 |
| I-377 | 0.4 | 0.47 | A | >2 | >2 |
| I-378 | 0.0017 | 0.0035 | A | >2 | >2 |
| I-379 | 0.026 | 0.056 | A | >2 | >2 |
| I-380 | 0.21 | 0.24 | A | >2 | >2 |
| I-381 | 0.0009 | 0.0027 | A | >2 | >2 |
| I-382 | 0.12 | 0.10 | A | >2 | >2 |
| I-383 | 0.019 | 0.012 | A | >2 | >2 |
| I-384 | 0.0025 | 0.0022 | A | >2 | >2 |
| I-385 | 0.0014 | 0.0012 | A | >2 | >2 |
| I-386 | 0.050 | 0.040 | A | >2 | >2 |
| I-387 | 0.012 | 0.01 | A | >2 | >2 |
| I-388 | 0.036 | 0.0285 | A | >2 | >2 |
| I-389 | 0.022 | 0.023 | A | >2 | >2 |
| I-390 | 0.019 | 0.0125 | A | >2 | >2 |
| I-391 | 0.0004 | 0.0004 | A | >1.01 | 1.01 |
| I-392 | 0.0038 | 0.0028 | A | >2 | >2 |
| I-393 | 0.0004 | 0.0003 | A | >1.01 | 0.86 |
| I-394 | 0.058 | 0.055 | A | >2 | >2 |
| I-395 | 0.056 | 0.0405 | A | >2 | >2 |
| I-396 | 0.0003 | 0.0003 | A | 1.01 | 1.06 |
| I-397 | 0.0006 | 0.0006 | A | >1.01 | 0.76 |
| I-398 | 0.0004 | 0.0005 | A | >1.01 | 1.01 |
| I-399 | 0.0004 | 0.0006 | A | >1.01 | 0.86 |
| I-400 | 0.0003 | 0.0004 | A | 1.01 | 0.81 |
| I-401 | 0.0003 | 0.0005 | A | 0.96 | 0.71 |
| I-402 | 0.021 | 0.015 | A | >2 | >2 |
| I-403 | 0.023 | 0.024 | A | >2 | >2 |
| I-404 | 0.025 | 0.016 | A | >2 | >2 |
| I-405 | 0.0018 | 0.0017 | A | 0.75 | 0.58 |
| I-406 | 0.01 | 0.0088 | A | >2 | >2 |
| I-407 | 0.017 | 0.017 | A | >2 | >2 |
| I-408 | 0.0007 | 0.001 | A | 0.86 | 0.51 |
| I-409 | 0.0003 | 0.0005 | A | >1.01 | 0.56 |
| I-410 | 0.0006 | 0.0009 | A | >1.01 | >1.01 |
| I-411 | 0.0002 | 0.0003 | A | >2 | >2 |
| I-412 | 0.0131 | 0.015 | A | >2 | >2 |
| I-413 | 0.0006 | 0.0008 | A | 0.81 | 0.81 |
| I-414 | 0.0085 | 0.016 | A | >2 | >2 |
| I-415 | 0.019 | 0.019 | A | >2 | >2 |
| I-416 | 0.0004 | 0.0007 | A | >1.63 | >1.63 |
| I-417 | 0.0002 | 0.0004 | A | >1.63 | >1.63 |
| I-418 | 0.0021 | 0.0056 | A | >1.25 | >1.25 |
| I-419 | 0.0011 | 0.003 | A | >1.25 | >1.25 |
| I-420 | 0.0046 | 0.0098 | A | >1.25 | >1.25 |
| I-421 | 0.0015 | 0.0034 | A | 0.6 | 0.75 |
| I-422 | 0.0002 | 0.0003 | A | 2 | 1.5 |
| I-423 | 0.0004 | 0.0007 | A | >2 | >2 |
| I-424 | 0.0001 | 0.0003 | A | 1.8 | 1.2 |
| I-425 | 0.0003 | 0.0004 | A | >2 | >2 |
| I-426 | 0.0001 | 0.0001 | A | 1.8 | 1.3 |
| I-427 | 0.0005 | 0.0008 | A | 0.42 | 0.52 |
| I-428 | 0.0006 | 0.0007 | A | 1.6 | 1.7 |
| I-429 | 0.0004 | 0.0005 | A | 0.4 | 0.44 |
| I-430 | 0.0006 | 0.0009 | A | >2 | >2 |
| I-431 | 0.0007 | 0.0009 | A | >2 | >2 |
| I-432 | 0.0002 | 0.0003 | A | 1.1 | 0.97 |
| I-433 | 0.0027 | 0.0034 | A | >2 | >2 |
| I-434 | 0.026 | 0.075 | A | >2 | >2 |
| I-435 | 0.0016 | 0.0038 | A | >2 | >2 |
| I-436 | 0.03 | 0.039 | A | >2 | >2 |
| I-437 | 0.0064 | 0.0078 | A | >2 | >2 |
| I-438 | 0.32 | 0.42 | A | >2 | >2 |
| I-439 | 0.078 | 0.096 | A | >2 | >2 |
| I-440 | 0.52 | 0.64 | A | >2 | >2 |
| I-441 | 0.076 | 0.13 | A | >2 | >2 |
| I-442 | >2 | >2 | A | >2 | >2 |
| I-443 | 0.0005 | 0.0006 | A | >2 | 1.6 |
| I-444 | 0.065 | 0.076 | A | >2 | >2 |
| I-445 | 0.26 | 0.26 | A | >2 | >2 |
| I-446 | 0.106 | 0.13 | A | >2 | >2 |
| I-447 | 0.46 | 0.59 | A | >2 | >2 |
| I-448 | 0.0002 | 0.0003 | A | 2 | 1.7857 |
| I-449 | 0.0009 | 0.0019 | A | >1.01 | 0.66 |
| I-450 | 0.0004 | 0.0008 | A | >1.01 | >1.01 |
| I-451 | 0.0067 | 0.0059 | A | >2 | >2 |
| I-452 | 0.0089 | 0.0066 | A | >2 | >2 |
| I-453 | 0.45 | 0.66 | A | >2 | >2 |
| I-454 | 0.041 | 0.067 | A | >2 | >2 |
| I-455 | 0.0012 | 0.0024 | A | 0.45 | 0.415 |
| I-456 | 0.0032 | 0.0042 | A | >2 | >2 |
| I-457 | 0.0008 | 0.0009 | A | 1.9 | 1.5 |
| I-458 | 0.0087 | 0.018 | A | 1.1533 | 1.19 |
| I-459 | 0.0018 | 0.0013 | A | 0.35 | 0.66 |
| I-460 | 0.0106 | 0.014 | A | 1.7333 | 1.8667 |
| I-461 | 0.0004 | 0.0005 | A | 0.66 | 0.56 |
| I-462 | 0.0006 | 0.0008 | A | 1.6 | 1.25 |
| I-463 | 0.0007 | 0.0009 | A | 1.01 | 0.86 |
| I-464 | 0.0005 | 0.0009 | A | 0.81 | 0.61 |
| I-465 | 0.0005 | 0.0012 | A | >1.01 | 0.96 |
| I-466 | 0.0002 | 0.0004 | A | 1.15 | 0.995 |
| I-467 | 0.0043 | 0.0077 | A | >2 | >2 |
| I-468 | 0.0025 | 0.0047 | A | >2 | >2 |
| I-469 | 0.0003 | 0.0006 | A | >2 | >2 |
| I-470 | 0.0057 | 0.0062 | A | >2 | >2 |
| I-471 | 0.0011 | 0.0014 | A | >2 | >2 |
| I-472 | 0.0339 | 0.02 | A | >2 | >2 |
| I-473 | 0.018 | 0.0277 | A | >2 | >2 |
| I-474 | 0.039 | 0.053 | A | >2 | >2 |
| I-475 | 0.026 | 0.034 | A | >2 | >2 |
| I-476 | 0.0007 | 0.0016 | A | >0.5 | >0.5 |
| I-477 | 0.0007 | 0.0018 | A | >0.5 | >0.5 |
| I-478 | 0.0019 | 0.0041 | A | >0.5 | >0.5 |
| I-479 | 0.0002 | 0.0003 | A | 1.5 | 1.0333 |
| I-480 | 0.0003 | 0.0007 | A | >1.25 | >1.25 |
| I-481 | 0.0003 | 0.0004 | A | >1.25 | >1.25 |
| I-482 | 0.0002 | 0.0002 | A | >2 | 1.2 |
| I-483 | 0.0036 | 0.0028 | A | >2 | >2 |
| I-484 | 0.0005 | 0.0011 | A | >1.1 | >1.1 |
| I-485 | 0.016 | 0.018 | A | >2 | >2 |
| I-486 | 0.042 | 0.07 | A | >2 | >2 |
| I-487 | 0.0002 | 0.0003 | A | >2 | >2 |
| I-488 | 0.0065 | 0.014 | A | >2 | >2 |
| I-489 | 0.115 | 0.27 | A | >2 | >2 |
| I-490 | 0.0031 | 0.0039 | A | >2 | >2 |
| I-491 | 0.19 | 0.23 | A | >2 | >2 |
| I-492 | 0.0075 | 0.0073 | A | >2 | >2 |
| I-493 | 0.0004 | 0.0004 | A | >2 | >2 |
| I-494 | >2 | 0.0001 | A | >2 | >2 |
| I-495 | 0.013 | 0.015 | A | >2 | >2 |
| I-496 | 0.0004 | 0.0006 | A | >2 | >2 |
| I-497 | 0.0022 | 0.0022 | A | >2 | >2 |
| I-498 | 0.0003 | 0.0003 | A | >2 | >2 |
| I-499 | 0.0036 | 0.005 | A | >2 | >2 |
| I-500 | 0.0004 | 0.0005 | A | >2 | >2 |
| I-501 | 0.0001 | 0.0001 | A | >2 | >2 |
| I-502 | 0.0002 | 0.0002 | A | >2 | >2 |
| I-503 | 0.0002 | 0.0003 | A | >2 | >2 |
| I-504 | 0.0012 | 0.0017 | A | >2 | >2 |
| I-505 | 0.014 | 0.0096 | A | >2 | >2 |
| I-506 | 0.023 | 0.021 | A | >2 | >2 |
| I-507 | 0.0006 | 0.0005 | A | >2 | >2 |
| I-508 | 0.0081 | 0.010 | A | >2 | >2 |
| I-509 | 0.0002 | 0.0005 | A | >2 | >2 |
| I-510 | 0.0003 | 0.0008 | A | >2 | >2 |
| I-511 | 0.043 | 0.0495 | A | >2 | >2 |
| I-512 | 0.019 | 0.018 | A | >2 | >2 |
| I-513 | 0.0012 | 0.0017 | A | >2 | >2 |
| I-514 | 0.0013 | 0.0011 | A | >2 | >2 |
| I-515 | 0.016 | 0.021 | A | >2 | >2 |
| I-516 | 0.0018 | 0.0027 | A | >2 | >2 |
| I-517 | 0.0014 | 0.0023 | A | >2 | >2 |
| I-518 | 0.010 | 0.017 | A | >2 | >2 |
| I-519 | 0.014 | 0.026 | A | >2 | >2 |
| I-520 | 0.0003 | 0.0004 | A | >2 | >2 |
| I-521 | 0.0007 | 0.0012 | A | >2 | >2 |
| I-522 | 0.0025 | 0.0026 | A | >2 | >2 |
| I-523 | 0.002 | 0.0019 | A | >2 | >2 |
| I-524 | 0.002 | 0.0026 | A | >2 | >2 |
| I-525 | 0.0007 | 0.0014 | A | 1.6 | 1.65 |
| I-526 | 0.24 | 0.147 | A | >2 | >2 |
| I-527 | 0.031 | 0.045 | A | >2 | >2 |
| I-528 | 0.27 | 0.25 | A | >2 | >2 |
| I-529 | 0.001 | 0.0012 | A | >2 | >2 |
| I-530 | 0.0068 | 0.005 | A | >2 | >2 |
| I-531 | 0.0015 | 0.0028 | A | >2 | >2 |
| I-532 | 0.0003 | 0.0003 | A | >2 | >2 |
| I-533 | 0.0007 | 0.0017 | A | >2 | >2 |
| I-534 | 0.0002 | 0.0001 | A | >2 | >2 |
| I-535 | 0.0021 | 0.0012 | A | >2 | >2 |
| I-536 | 0.0023 | 0.0022 | A | >2 | >2 |
| I-537 | 0.0003 | 0.0004 | A | >2 | >2 |
| I-538 | 0.0006 | 0.0006 | A | 1.5 | 1.47 |
| I-539 | 0.0015 | 0.0022 | A | >2 | >2 |
| I-540 | 0.0003 | 0.0003 | A | >2 | >2 |
| I-541 | 0.0015 | 0.001 | A | >2 | >2 |
| I-542 | 0.032 | 0.028 | A | >2 | >2 |
| I-543 | 0.0006 | 0.0008 | A | >2 | >2 |
| I-544 | 0.0025 | 0.0033 | A | >2 | >2 |
| I-545 | 0.0006 | 0.0009 | A | >2 | >2 |
| I-546 | 0.0054 | 0.0074 | A | >2 | >2 |
| I-547 | 0.00009 | 0.0001 | A | >2 | >2 |
| I-548 | 0.69 | 0.64 | A | >2 | 1.8 |
| I-549 | 1.1 | 1.4 | A | >2 | >2 |

*Method refers to the maximal concentration of antagonist present for SLIGKV and Trypsin activation: Method A: 0.1, 1 or 2 μM; Method B: 50 μM.
*Method refers to the maximal concentration of antagonist present for SLIGKV and Trypsin activation: Method A: 0.1, 1 or 2 μM; Method B: 50 μM.

### Example 99: Protocol for Testing PAR2 Compounds In Vivo Rat Pharmacokinetics Experiments

Compounds delivered by intravenous route were formulated using a solution formulation consisting of 5% N-methylpyrrolidone 10% Vitamin E-TPGS and 85% water (v/v/) or a solution formulation consisting of 25% N-methylpyrrolidone 35% polyethylene glycol 400 and 40% water (v/v/). Intravenous formulations were administered by a bolus injection (over approximately 10 seconds) using a hypodermic needle attaches to a syringe (tail vein). The dose volume was varied from 1 to 2 mL/kg and the dose was varied from 0.5 to 1 mg/kg. Compounds delivered orally were formulated as a solution formulation consisting of 5% N-methylpyrrolidone 10% Vitamin E-TPGS and 85% water (v/v/) or as fine suspension using a cellulose derivative suspending agent with or without the addition of a wetting agent/surfactant. Milling of large particles was performed as needed to reduce particle size using focused electroacoustic milling, homogenization or low energy media milling. A spray-dried solid dispersion was prepared for key compounds using 1:1 combination with cellulose derivatives. Test compounds were administered orally by gavage using a gavage plastic needle attached to a syringe. The dose volume was varied from 5 to 10 mL/kg and the dose was varied from 1 to 600 mg/kg.

Rats were bled by venipuncture (jugular vein) and the samples were collected into tubes containing K₃EDTA. The tubes were placed on wet ice until processed. The samples were centrifuged for approximately 10 minutes (at approximately 4° C.) within approximately 30 minutes of collection to prepare plasma or diluted in citrate buffer (1:3) and stored frozen (approximately −30° C.). Thawed plasma or whole blood samples were extracted by protein precipitation using an organic solvent followed by centrifugation. The supernatants were separated by high-performance liquid chromatography coupled to mass spectrometry detection. A calibration curve was prepared using reference standard compounds spiked into either blank plasma or whole blood matrices and extracted similarly to unknown samples. Pharmacokinetic parameters were calculated using non-compartmental analysis.

TABLE 3
Compound pharmacokinetic data in rat

| Compound | Clₚ | T ½ | | |
|---|---|---|---|---|
| number | (mL/min/kg) | (h) | L/kg | F (%) |
| I-316 | 13.9 | 1.0 | 0.64 | 49 |
| I-287 | 5.2 | 3.7 | 0.89 | 58 |
| I-448 | 9.3 | 5.2 | 0.83 | 125 |
| I-429 | 5.5 | 4.0 | 0.79 | 36 |
| I-427 | 10.6 | 1.9 | 0.78 | 79 |
| I-449 | 5.0 | 5.3 | 1.7 | 119 |
| I-423 | 18.6 | 0.44 | 0.48 | 77 |
| I-422 | 12.8 | 0.86 | 0.53 | 18 |
| I-352 | 6.5 | 5.6 | 1.6 | 48 |
| I-346 | 16.5 | 0.44 | 0.17 | 4.7 |
| I-345 | 7.6 | 1.8 | 0.64 | 52 |
| I-344 | 10.2 | 0.99 | 0.44 | 24 |
| I-343 | 53 | 0.33 | 1.1 | 27 |
| I-411 | 6.5 | 2.0 | 0.76 | 70 |
| I-406 | 3.6 | 23.2 | 6.8 | 66 |
| I-277 | 5.1 | 14.9 | 5.7 | 78 |
| I-260 | 3.4 | 30.7 | 4.1 | 87 |
| I-367 | 28 | 0.32 | 0.71 | 2.7 |
| I-354 | 32 | 0.57 | 1.8 | 52 |
| I-324 | 21.3 | 0.47 | 0.74 | 19 |
| I-465 | 9.2 | 5.1 | 1.3 | 46 |
| I-338 | 33 | 0.5 | 0.97 | |
| I-331 | 8.9 | 1.6 | 0.21 | |

### Example 100: In Vivo Pharmacological Evaluation of PAR2 Pathway Inhibitors Subjects and Housing and Rat Carrageenan-Induced Paw Edema Model

Male Sprague Dawley rats were housed in groups in a temperature controlled room and were acclimatized in the animal facility for at least three days prior to use. Experiments were performed during the light phase of the cycle. Animals have food and water ad libitum.

Peripheral inflammation was induced by intra-plantar administration of 100 ul of a 1% w/volume (in saline) carrageenan solution under isoflurane anesthesia. The degree of swelling was assessed using the plethysmometer at 2-3-4-5-6 h after carrageenan injection. Test compound or vehicle (0.5% MC/0.1% SDS) was systemically administered at the indicated doses in a volume of 5 mL/kg either 0.5-3 hour before carrageenan challenge (Table 4).

TABLE 4
Inhibition of carrageenan induced paw edema in rats

| Compound | Dose (mg/kg) | % inhibition |
|---|---|---|
| I-316 | 20 | 38 |
| I-287 | 200 | 30 |
| I-448 | 0.8 | 11 |
| | 3 | 18 |
| | 10 | 30 |
| | 20 | 21 |
| I-429 | 50 | 60 |
| I-427 | 50 | 46 |
| I-449 | 15 | 22 |
| | 40 | 49 |
| | 100 | 73 |
| I-316 | 5 | 25 |
| | 15 | 50 |
| | 40 | 66 |
| | 100 | 81 |
| | 250 | 79 |
| I-423 | 20 | 56 |
| I-422 | 20 | 32 |
| I-421 | 20 | 59 |
| I-352 | 20 | 61 |
| I-346 | 20 | 60 |
| I-345 | 15 | 28 |
| I-344 | 20 | 43 |
| I-343 | 20 | 29 |
| I-411 | 8 | 36 |
| I-406 | 20 | 21 |
| I-277 | 20 | 61 |
| I-260 | 20 | 0.4 |
| I-367 | 20 | 68 |
| I-354 | 20 | 60 |
| I-324 | 20 | 71 |
| I-465 | 20 | 6 |
| I-358 | 20 | 61 |
| I-338 | 20 | 0 |
| I-331 | 20 | 27 |

While we have described a number of embodiments of this invention, it is apparent that our basic examples may be altered to provide other embodiments that utilize the compounds, methods, and processes of this invention. Therefore, it will be appreciated that the scope of this invention is to be defined by the appended claims rather than by the specific embodiments that have been represented by way of example herein.

All references provided herein are incorporated herein in its entirety by reference. As used herein, all abbreviations, symbols and conventions are consistent with those used in the contemporary scientific literature. See. e.g., Janet S. Dodd, ed., The ACS Style Guide: A Manual for Authors and Editors, 2nd Ed., Washington, D.C.: American Chemical Society. 1997.

1. A compound represented by Structural Formula (I) or a pharmaceutically acceptable salt thereof: 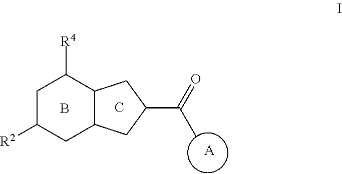
wherein
Ring B-C is selected from the group consisting of 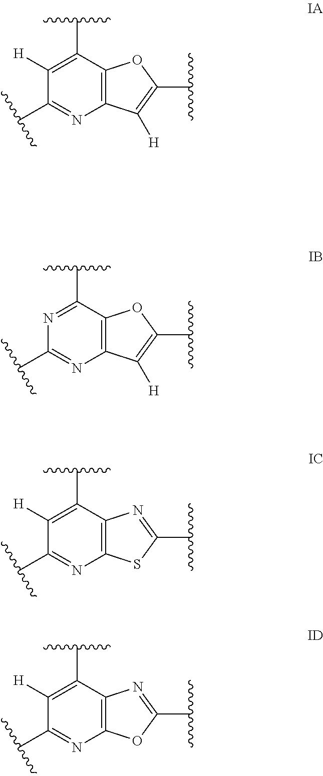 [SMILES: *c1cc(*)c2oc(*)cc2n1 ; *c1nc(*)c2oc(*)cc2n1 ; *c1cc(*)c2nc(*)sc2n1 ; *c1cc(*)c2nc(*)oc2n1]
Ring A is 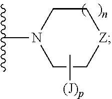
wherein
n is 1 or 2;
Z is —O—, —CH₂—, —NX—, or —CRX³;
X is R⁵, —C(O)R⁵, or —S(O)₂R⁵;
X³ is —(CR₂)ᵣ—C(O)OR⁶, —(CR₂)ᵣ—N(R)R⁶, —(CR₂)ᵣ—C(O)N(R)R⁶ or —(CR₂)ᵣ—C(O)N(R)S(O)₂R⁶; or optionally X³ and J, together with the atoms to which they are bound, form a 5-6 membered aromatic monocyclic ring having 0-2 heteroatoms selected from oxygen, nitrogen, or sulfur: wherein said 5-6 membered ring forms a fused ring together with Ring A, and is optionally substituted with 1-4 occurrences of substituents selected from oxo, halogen, CN, —OH, —O(C₁₋₄ alkyl), —O(haloC₁₋₄ alkyl), C₁₋₄ alkyl, or haloC₁₋₄ alkyl;
J is CN, oxo, a C₁₋₆aliphatic group wherein up to three carbon units of said C₁₋₆aliphatic can each be optionally and independently replaced with —O—, —NR—, —S—, or —C(O)—; or a 3-7 membered, saturated, partially unsaturated or aromatic, monocyclic ring having 0-4 heteroatoms selected from oxygen, nitrogen, or sulfur; wherein said J is optionally and independently substituted with 1-4 occurrences of halogen, CN, or C₁₋₄alkyl, wherein up to one methylene unit of said C₁₋₄alkyl is optionally replaced with —O—, —NR—, or —S—, and wherein said C₁₋₄alkyl is optionally substituted with 1-4 occurrences of halogen or CN;
or two J groups on the same or different atom(s), together with the atom(s) to which they are bound, form a 3-6 membered, saturated monocyclic ring having 0-2 heteroatoms selected from oxygen, nitrogen, or sulfur; wherein said 3-6 membered ring is optionally substituted with one occurrence of oxo;
p is 0, 1, 2, 3, or 4;
each r is independently 0, 1 or 2;
each of R⁵ and R⁶ is independently —(V)ₐ—Y; wherein
V is C₁₋₆aliphatic wherein up to three carbon units of said C₁₋₆aliphatic can each be optionally and independently replaced with —O—, —NR—, —S—, —C(O)— or —S(O)₂—; wherein V is optionally substituted with 1-4 occurrences of J^{V};
J^{V} is halogen, CN, haloC₁₋₄alkyl, or C₁₋₄alkyl, wherein up to one methylene unit of each of said C₁₋₄alkyl and haloC₁₋₄alkyl is optionally replaced with —O—, —NR—, —S—, or —C(O)—;
Y is H, —CN, a 3-7 membered, saturated, partially unsaturated or aromatic, monocyclic ring having 0-4 heteroatoms selected from oxygen, nitrogen, or sulfur; or a 6-10 membered, saturated, partially unsaturated or aromatic, bicyclic ring having 0-6 heteroatoms selected from oxygen, nitrogen, or sulfur: wherein Y is optionally substituted with 1-4 occurrences of J^{Y};
J^{Y} is H; oxo; halogen; —CN: phenyl; 5-6-membered heteroaryl having 1-4 heteteroatoms selected from oxygen, nitrogen, or sulfur; or C₁₋₆aliphatic, wherein up to three carbon units of said C₁₋₆aliphatic can each be optionally and independently replaced with —O—, —NR—, —S—, —C(O)—, or —S(O)₂—; and wherein each of the phenyl, 5-6 membered heteroaryl and the C₁₋₆aliphatic is optionally and independently substituted with 1-4 occurrences of substituents selected from the group consisting of halogen, —CN, —OH, —OCH₃, —C(O)OH, —OP(O)(OH)₂, —P(O)(R)(OH), or 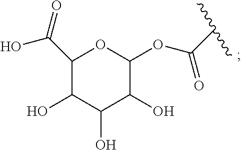 [SMILES: *C(=O)OC1OC(C(=O)O)C(O)C(O)C1O]
and
each R is independently H or C₁₋₄alkyl;
R² is —(V)_{b}—Y²: wherein
V² is a C₁₋₄aliphatic;
Y² is halogen; C₁₋₆aliphatic; a 3-7 membered, saturated, partially unsaturated or aromatic, monocyclic ring having 0-4 heteroatoms selected from oxygen, nitrogen, or sulfur: or a 6-10 membered, saturated, partially unsaturated or aromatic, bicyclic ring having 0-6 heteroatoms selected from oxygen, nitrogen, or sulfur: wherein Y² is optionally substituted with 1-4 occurrences of J^{Y}; and
a and b are each independently 0 or 1;
R⁴ is halogen: —CN; C₁₋₆aliphatic therein up to three carbon units of said C₁₋₆aliphatic can each be optionally and independently replaced with —O—, —NR—, —S— or —C(O)—; a 3-7 membered saturated, partially saturated, or aromatic monocyclic ring having 0-4 heteroatoms selected from oxygen, nitrogen, or sulfur; or a 6-10 membered, saturated, partially unsaturated or aromatic, bicyclic ring having 0-6 heteroatoms selected from oxygen, nitrogen, or sulfur; wherein said R⁴ is optionally and independently substituted with 1-4 occurrences of oxo, halogen, CN, or C₁₋₆aliphatic wherein up to three carbon units of said C₁₋₆aliphatic can each be optionally and independently replaced with —O—, —NR—, —S— or —C(O)—.

2. A compound represented by Structural Formula (I) or a pharmaceutically acceptable salt thereof: 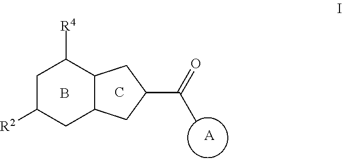
wherein
Ring B-C is selected from the group consisting of 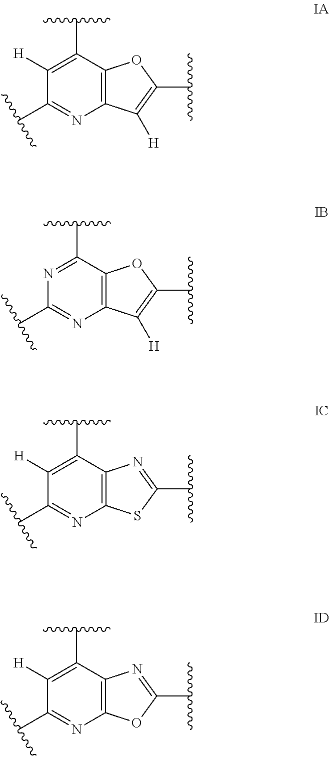 [SMILES: *c1cc(*)c2oc(*)cc2n1 ; *c1nc(*)c2oc(*)cc2n1 ; *c1cc(*)c2nc(*)sc2n1 ; *c1cc(*)c2nc(*)oc2n1]
Ring A is 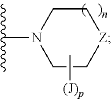
wherein
n is 1 or 2;
Z is —O—, —CH₂—, or —NX—;
X is R⁵, —C(O)R⁵, or —S(O)₂R⁵;
J is CN, oxo, a C₁₋₆aliphatic group wherein up to three carbon units of said C₁₋₆aliphatic can each be optionally and independently replaced with —O—, —NR—, —S— or —C(O)—; or a 3-7 membered, saturated, partially unsaturated or aromatic, monocyclic ring having 0-4 heteroatoms selected from oxygen, nitrogen, or sulfur; wherein said J is optionally and independently substituted with 1-4 occurrences of halogen, CN, or C₁₋₄alkyl, wherein up to one methylene unit of said C₁₋₄alkyl is optionally replaced with —O—, —NR—, or —S—, and wherein said C₁₋₄alkyl is optionally substituted with 1-4 occurrences of halogen or —CN;
or two J groups on the same or different atom(s), together with the atom(s) to which they are bound, form a 3-6 membered, saturated, monocyclic ring having 0-2 heteroatoms selected from oxygen, nitrogen, or sulfur; wherein said 3-6 membered ring is optionally substituted with one occurrence of oxo;
p is 0-4;
each R⁵ is independently —(V)ₐ—Y: wherein
V is C₁₋₆aliphatic wherein up to three carbon units of said C₁₋₆aliphatic can each be optionally and independently replaced with —O—, —NR—, —S—, or —C(O)—; wherein V is optionally substituted with 1-4 occurrences of J^{V};
J^{V} is halogen, CN, or C₁₋₄alkyl, wherein up to one methylene unit of said C₁₋₄alkyl is optionally replaced with —O—, —NR—, or —S—;
Y is H, a 3-7 membered, saturated, partially unsaturated or aromatic, monocyclic ring having 0-4 heteroatoms selected from oxygen, nitrogen, or sulfur; or a 6-10 membered, saturated, partially unsaturated or aromatic, bicyclic ring having 0-6 heteroatoms selected from oxygen, nitrogen, or sulfur; wherein Y is optionally substituted with 1-4 occurrences of J^{Y};
J^{Y} is H, oxo, halogen, CN, phenyl, or C₁₋₆aliphatic, wherein up to three carbon units of said C₁₋₆aliphatic can each be optionally and independently replaced with —O—, —NR—, —S—, or —C(O)—; and wherein each of the phenyl and the C₁₋₆aliphatic is optionally and independently substituted with 1-4 occurrences of halogen or —CN; and
each R is independently H or C₁₋₄alkyl;
R² is —(V²)_{b}—Y²; wherein
V² is a C₁₋₄aliphatic;
Y² is halogen; C₁₋₆aliphatic; or a 3-7 membered, saturated, partially unsaturated or aromatic, monocyclic ring having 0-4 heteroatoms selected from oxygen, nitrogen, or sulfur: wherein Y² is optionally substituted with 1-4 occurrences of J^{Y}; and
a and b are each independently 0 or 1;
R⁴ is halogen: CN; C₁₋₆aliphatic wherein up to three carbon units of said C₁₋₆aliphatic can each be optionally and independently replaced with —O—, —NR—, —S— or —C(O)—; a 3-7 membered saturated, partially saturated, or aromatic monocyclic ring having 0-4 heteroatoms selected from oxygen, nitrogen, or sulfur; or a 6-10 membered, saturated, partially unsaturated or aromatic, bicyclic ring having 0-6 heteroatoms selected from oxygen, nitrogen, or sulfur; wherein said R⁴ is optionally and independently substituted with 1-4 occurrences of oxo, halogen, —CN, or C₁₋₆aliphatic wherein up to three carbon units of said C₁₋₆aliphatic can each be optionally and independently replaced with —O—, —NR—, —S— or —C(O)—.

3. The compound of claim 1 or 2, wherein Z is —NX—.

4. The compound of claim 3, wherein J is methyl.

5. The compound of any one of claims 1-4, wherein p is 1 or 2.

6. The compound of any one of claims 1-5, wherein n is 1.

7. The compound of claim 1, wherein Ring A is 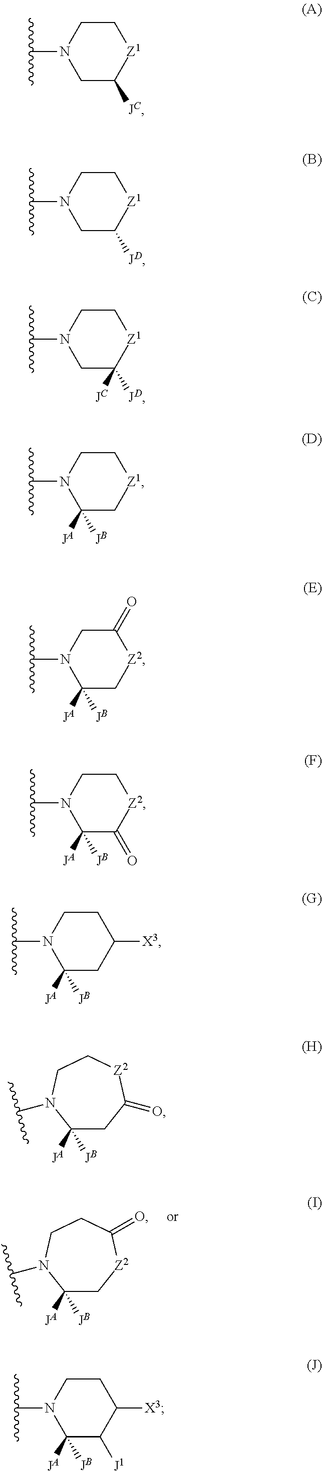
wherein
each Z¹ is independently —O—, —CH₂—, or —NX—; wherein X is X¹;
each Z² is independently —CH₂— or —NX—; wherein X is X²;
X¹ is R⁵, —C(O)R⁵, or —S(O)₂R⁵;
X² is R⁵;
J^{A} is C₁₋₄alkyl;
J^{B} is C₁₋₄alkyl;
or J^{A} and J^{B}, together with the carbon atom to which they are bound, form a 3-6 membered saturated monocyclic ring having 0-1 heteroatom selected from oxygen, nitrogen, or sulfur; and
X³ in formula (G) is —(CR₂)ᵣC(O)OR⁶, —(CR₂)ᵣ—N(R)R⁶, —(CR₂)ᵣ—C(O)N(R)R⁶ or —(CR₂)ᵣ—C(O)N(R)S(O)₂R⁶;
J¹ and X³ in formula (J), together with the atoms to which they are bound, form a 5-6 membered, aromatic, monocyclic ring having 1-2 heteroatoms selected from oxygen, nitrogen, or sulfur, wherein said 5-6 membered ring forms a fused ring together with Ring A, and is optionally substituted with 1-4 occurrences of substituents selected from oxo, halogen, CN, —OH, —O(C₁₋₄ alkyl), —O(haloC₁₋₄ alkyl), C₁₋₄ alkyl, or haloC₁₋₄alkyl;
each R⁶ is independently —H, C₁₋₆ aliphatic or a 3-7 membered, saturated, partially unsaturated or aromatic, monocyclic ring having 0-4 heteroatoms selected from oxygen, nitrogen, or sulfur, wherein said C₁₋₆ aliphatic is optionally substituted with 1-4 occurrences of J^{V}, and wherein said monocyclic ring is optionally substituted with 1-4 occurrences of J^{Y}.
J^{C} is methyl; and
J^{D} is methyl.

8. The compound of claim 2, wherein Ring A is 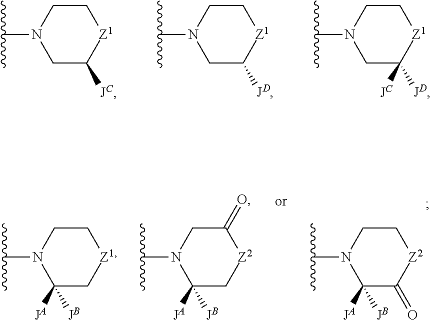
wherein
each Z¹ is independently —O—, —CH₂—, or —NX—; wherein X is X¹;
each Z² is independently —CH₂— or —NX—: wherein X is X²;
X¹ is R⁵, —C(O)R⁵, or —S(O)₂R⁵;
X² is R⁵;
each R⁵ is independently —(V)ₐ—Y; wherein
V is C₁₋₆aliphatic wherein up to three carbon units of said C₁₋₆aliphatic can each be optionally and independently replaced with —O—, —NR—, —S—, or —C(O)—; wherein V is optionally substituted with 1-3 occurrences of halogen, C₁₋₄alkyl, OH, NH₂, or —NRC(O)C₁₋₄alkyl;
Y is H or a 3-7 membered saturated, partially unsaturated, or aromatic monocyclic ring having 0-4 heteroatoms selected from oxygen, nitrogen, or sulfur; wherein Y is optionally substituted with 1-4 occurrences of J^{Y};
J^{Y} is H, oxo, CN, halogen, phenyl, or C₁₋₆aliphatic, wherein up to three carbon units of said C₁₋₆aliphatic can each be optionally and independently replaced with —O—, —NR—, —S— or —C(O)—; and wherein the C₁₋₆aliphatic is optionally substituted with 1-4 halogen;
each R is independently H or C₁₋₄alkyl;
a is 0 or 1;
J^{A} is C₁₋₄alkyl;
J^{B} is C₁₋₄alkyl;
or J^{A} and J^{B}, together with the carbon atom to which they are bound, form a 3-6 membered saturated monocyclic ring having 0-1 heteroatom selected from oxygen, nitrogen, or sulfur; and
J^{C} is methyl; and
J^{D} is methyl.

9. The compound of claim 8, wherein Ring A is

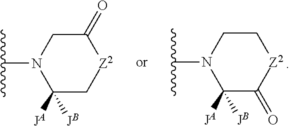

10. The compound of claim 8, wherein Ring A is

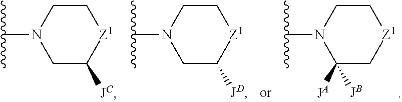

11. The compound of claim 10, wherein Ring A is

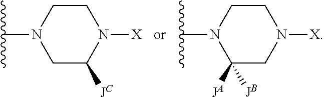

12. The compound of claim 7, wherein Ring A is

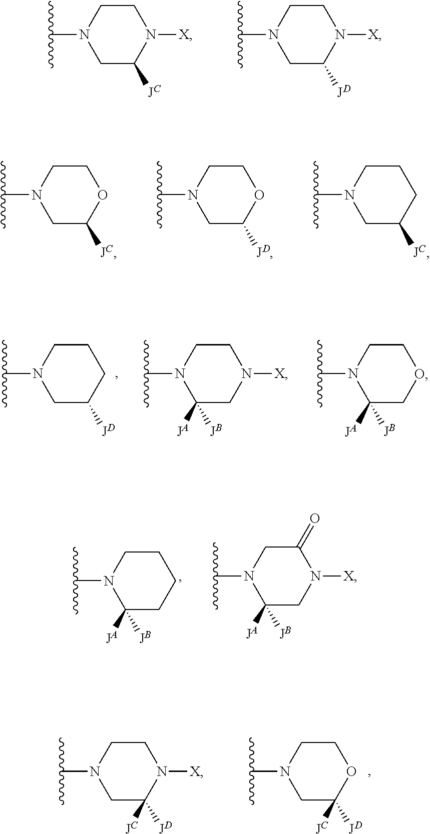

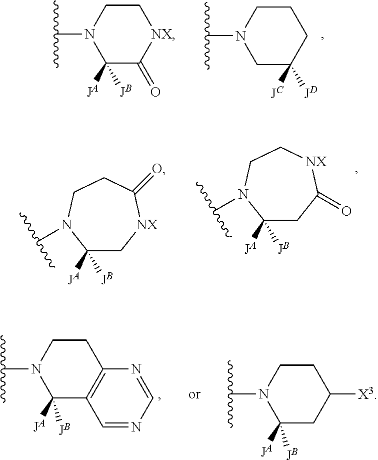

13. The compound of claim 8, wherein Ring A is

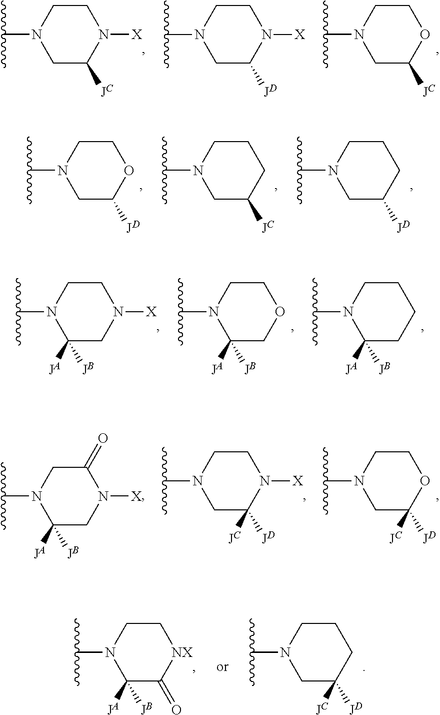

14. The compound of any one of claims 9-13, wherein R⁵ is —(V)ₐ—Y: wherein
V is C₁₋₆aliphatic wherein up to three carbon units of said C₁₋₆aliphatic can each be optionally and independently replaced with —O—, —NR—, —S—, —C(O)—, or —S(O)₂; wherein V is optionally substituted with 1-3 occurrences of halogen, C₁₋₄alkyl, OH, NH₂, or —NRC(O)C₁₋₄alkyl; and
Y is H, cyclopropyl, cyclobutyl, cyclopentyl, cxyclohexyl, oxetanyl, azetidinyl, tetrahydrofuranyl, pyrrolidinyl, tetrahydropyranyl, piperidinyl, piperazinyl, thiomorpholinyl, morpholinyl, imidazolyl, isoxazolyl, pyrazolyl, pyridinyl, pyrazinyl, pyridazinyl, phenyl, oxadiazolyl, thienyl, pyrimidinyl, triazolyl, tetrazolyl, oxazolyl, thiazolyl, spiro[3,3]hepatanyl, or 1,1-dioxide-isothiazolidinyl; wherein Y is optionally and independently substituted with 1-4 occurrences of J^{Y};
J^{Y} is H, oxo, CN, halogen, C₁₋₄alkyl, haloC₁₋₄alkyl, —OH, —O(C₁₋₄alkyl), NH(C₁₋₄alkyl), N(C₁₋₄alkyl)₂, S(O)₂(C₁₋₄alkyl), phenyl, NH₂, tetrazole, (CH₂)ₜ—C(O)OH, (CH₂)ₜ—C(O)O(C₁₋₂alkyl), (CH(C₁₋₂ alkyl))ₜ—C(O)OH, (CH(C₁₋₂ alkyl))ₜ—C(O)O(C₁₋₂ alkyl), (C(C₁₋₂ alkyl)₂)ₜ—C(O)OH, (C(C₁₋₂ alkyl)₂)ₜ—C(O)O(C₁₋₂ alkyl), or —C(O)O—CH₂—O—P(O)(OH)₂;
a is 0 or 1; and
each t is independently 0 or 1.

15. The compound of any one of claims 9-13, wherein R⁵ is —(V)ₐ—Y; wherein
V is C₁₋₆aliphatic wherein up to three carbon units of said C₁₋₆aliphatic can each be optionally and independently replaced with —O—, —NR—, —S—, or —C(O)—; wherein V is optionally and independently substituted with 1-3 occurrences of halogen, C₁₋₄ alkyl, OH, NH₂, or —NRC(O)C₁₋₄alkyl; and
Y is H, cyclopropyl, cyclobutyl, cyclopentyl, cxyclohexyl, oxetanyl, azetidinyl, tetrahydrofuranyl, pyrrolidinyl, tetrahydropyranyl, piperidinyl, piperazinyl, thiomorpholinyl, morpholinyl, imidazolyl, isoxazolyl, pyrazolyl, pyridinyl, pyrazinyl, pyridazinyl, or phenyl; wherein Y is optionally and independently substituted with 1-4 occurrences of J^{Y};
J^{Y} is H, oxo, CN, halogen, C₁₋₄alkyl, haloC₁₋₄alkyl, OH, O(C₁₋₄alkyl), NH(C₁₋₄alkyl), N(C₁₋₄alkyl)₂, or phenyl; and
a is 0 or 1.

16. The compound of claim 1, 7, or 12, wherein:
X³ is —CR₂—C(O)OR⁶, —N(R)R⁶, —CR₂—C(O)N(R)R⁶ or —CR₂—C(O)N(R)S(O)₂R⁶.

17. The compound of claim 16, wherein X³ is —CH₂—C(O)OH, —NH₂, —NH(C₁₋₆ alkyl), —CH₂—C(O)NH—C₁₋₄ alkyl, —CH₂—C(O)NHS(O)₂C₁₋₆ alkyl, or —CH₂—C(O)NH—CN, wherein each of said C₁₋₆ alkyl is optionally and independently substituted with 1-4 occurrences of substituents selected from CN, halogen, C₁₋₄alkyl, haloC₁₋₄alkyl, —OH, —O(C₁₋₄alkyl), NH(C₁₋₄alkyl), N(C₁₋₄alkyl)₂, S(O)₂(C₁₋₄alkyl), NH₂, (CH₂)ₜ—C(O)OH, (CH₂)ₜ—C(O)O(C₁₋₂ alkyl), (CH(C₁₋₂ alkyl))ₜ—C(O)OH, (CH(C₁₋₂ alkyl))ₜ—C(O)O(C₁₋₂ alkyl), (C(C₁₋₂ alkyl)₂)ₜ—C(O)OH, or (C(C₁₋₂ alkyl)₂)ₜ—C(O)O(C₁₋₂ alkyl), wherein each t is independently 0 or 1.

18. The compound of any one of claims 1, 2, 11, 12, and 13 wherein X is —R⁵ or C(O)R⁵.

19. The compound of any one of claims 1, 2, 11, 12, and 13, wherein:
X is C₁₋₆alkyl, —Y, —C(O)—C₁₋₆ alkyl; —S(O)₂—C₁₋₃ alkyl; —C(O)—(CH₂)_{q}—C₁₋₃ alkyl; —C(O)N(R)S(O)₂—C₁₋₃ alkyl; —C(O)—(CH₂)_{q}—N(R)S(O)₂—C₁₋₃ alkyl; —C(O)N(R)S(O)₂—C₁₋₃ alkyl; —C(O)—(CH₂)_{q}—S(O)₂—N(R)—C₁₋₂ alkyl; —C(O)N(R)—C₁₋₃ alkyl; —C(O)—(CH₂)_{q}—S(O)₂—C₁₋₃ alkyl; —C(O)Y; —S(O)₂Y; —C(O)—(CH₂)_{q}—Y; —C(O)N(R)S(O)₂Y; —C(O)—(CH₂)_{q}—N(R)S(O)₂Y; —C(O)N(R)S(O)₂Y; —C(O)—(CH₂)_{q}—S(O)₂—N(R)Y; —C(O)N(R)Y: —C(O)—(CH₂)_{q}—S(O)₂Y;
each q is independently 1 or 2;
wherein each of said C₁₋₆ alkyl, C₁₋₃ alkyl and C₁₋₂ alkyl is optionally and independently substituted with 1-4 occurrences of J^{V}, each independently selected from CN, halogen, C₁₋₄alkyl, haloC₁₋₄alkyl, —OH, —O(C₁₋₂alkyl), —NH(C₁₋₂alkyl), —N(C₁₋₂alkyl)₂, —S(O)₂(C₁₋₂alkyl), —NH₂, —C(O)OH, —CH₂—C(O)OH, —(CH₂)₂—C(O)OH, —CH₂—C(O)OCH₃, or —C(O)O(C₁₋₂ alkyl);
each Y is independently optionally substituted with 1-4 occurrences of J^{Y}, each independently selected from oxo, CN, halogen, C₁₋₄alkyl, haloC₁₋₄alkyl, —OH, —O(C₁₋₄alkyl), NH(C₁₋₄alkyl), N(C₁₋₄alkyl)₂, S(O)₂(C₁₋₄alkyl), phenyl, NH₂, tetrazole, (CH₂)ₜ—C(O)OH, (CH₂)ₜ—C(O)O(C₁₋₂ alkyl), (CH(C₁₋₂ alkyl))ₜ—C(O)OH, (CH(C₁₋₂ alkyl))ₜ—C(O)O(C₁₋₂ alkyl), (C(C₁₋₂ alkyl)₂)ₜ—C(O)OH, (C(C₁₋₂ alkyl)₂)ₜ—C(O)O(C₁₋₂ alkyl), or —C(O)O—CH₂—O—P(O)(OH)₂, wherein each t is independently 0 or 1.

20. The compound of claim 19, wherein each Y is independently H, cyclopropyl, cyclobutyl, cyclopentyl, cyclohexyl, oxetanyl, azetidinyl, tetrahydrofuranyl, pyrrolidinyl, tetrahydropyranyl, piperidinyl, piperazinyl, thiomorpholinyl, morpholinyl, imidazolyl, isoxazolyl, pyrazolyl, pyridinyl, pyrazinyl, pyridazinyl, phenyl, pyrimidinyl, triazolyl, tetrazolyl, oxazolyl, thiazolyl, spiro[3,3]hepatanyl, or 1,1-dioxide-isothiazolidinyl; wherein Y is optionally and independently substituted with 1-4 occurrences of J^{Y}.

21. The compound of claim 20, wherein Y is optionally and independently substituted with 1-4 occurrences of J^{Y}, wherein J^{Y} is each independently —CN, halogen, —CH₃, —CF₃, —OH, —OCH₃, —NH₂, —NHCH₃, N(CH₃)₂, —C(O)OH, or —C(O)O(C₁₋₂ alkyl); and J^{V} is —CN, halogen, —CH₃, —CF₃, —OH, —OCH₃, —NH₂, —NHCH₃, N(CH₃)₂, —C(O)OH, or —C(O)O(C₁₋₂ alkyl).

22. The compound of any one of claims 1-21, wherein R² is —(V²)_{b}—Y², and b is 0.

23. The compound of claim 22, wherein Y² is a C₅₋₇ cycloalkyl group or 5-6 membered aromatic monocyclic ring having 0-4 heteroatoms selected from oxygen, nitrogen, or sulfur: wherein Y² is optionally substituted with 1-4 occurrences of J^{Y}.

24. The compound of claim 23, wherein Y² is a phenyl optionally substituted with 1-4 occurrences of halogen.

25. The compound of claim 23, wherein Y² is a C₅₋₇ cycloalkyl group optionally substituted with 1-4 occurrences of halogen or C₁₋₄alkyl.

26. The compound of claim 22, wherein Y² is a non-aromatic, 5-7-membered, heterocycylic ring having 1-2 heteroatoms selected from oxygen, nitrogen, or sulfur, and optionally substituted with 1-4 occurrences of halogen or C₁₋₄alkyl.

27. The compound of claim any one of claims 1-26, wherein R⁴ is C₁₋₆alkyl; cyclopropyl optionally substituted with one occurrence of CN or CH₃; or halogen.

28. The compound of claim 27, wherein R⁴ iso-propyl, tert-butyl, or cyclopropyl optionally substituted with one occurrence of CN or CH₃.

29. The compound of claim 1, represented by Structural Formula II or III, or a pharmaceutically acceptable salt thereof: 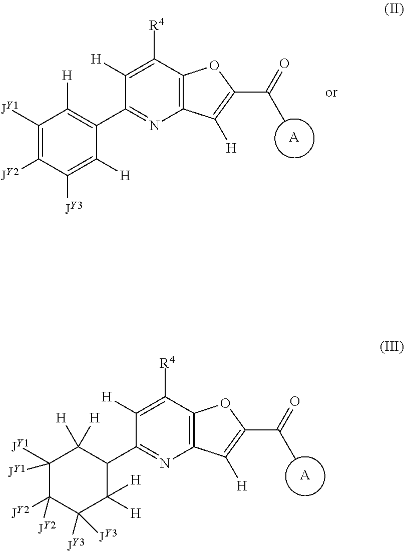
wherein each of J^{Y1}, J^{Y2}, and J^{Y3} is independently as defined for J^{Y}; and J^{Y}, R⁴, and Ring A are independently as defined in claim 1.

30. The compound of claim 29, wherein
Ring A is 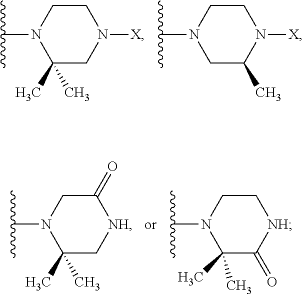
R⁴ is iso-propyl, tert-butyl, methylcyclopropyl, or cyanocyclopropyl;
X is R⁵ or —C(O)R⁵;
each R⁵ is independently (V)ₐ—Y; wherein
V is C₁₋₆aliphatic wherein up to two carbon units of said C₁₋₆aliphatic can each be optionally and independently replaced with —O— or —C(O)—; wherein V is optionally substituted with 1-3 occurrences of halogen, CN, C₁₋₄alkyl, OH, O(C₁₋₄alkyl), NH₂, or —NHC(O)C₁₋₄alkyl; and
Y is H, a 3-6 membered cycloalkyl, isoxazolyl, oxadiazolyl, pyrazolyl, imidazolyl, triazolyl, thienyl, pyrazolyl, tetrahydrofuranyl, tetrahydropyranyl, pyridinyl, pyrimidinyl, phenyl, oxetanyl, azetidinyl, pyrrolidinyl, piperidinyl, piperazinyl, thiomorpholinyl, morpholinyl, pyrazinyl, pyridazinyl, tetrazolyl, oxazolyl, thiazolyl, spiro[3,3]hepatanyl, or 1,1-dioxide-isothiazolidinyl; wherein Y is optionally substituted with 1-4 occurrences of J^{Y}; and
each J^{Y1}, J^{Y2}, and J^{Y3} is independently H, halogen, CN, C₁₋₄alkyl, haloC₁₋₄alkyl, or OH.

31. The compound of claim 29, wherein:
Ring A is 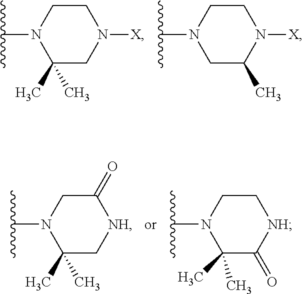
each X is independently selected from the group consisting of: 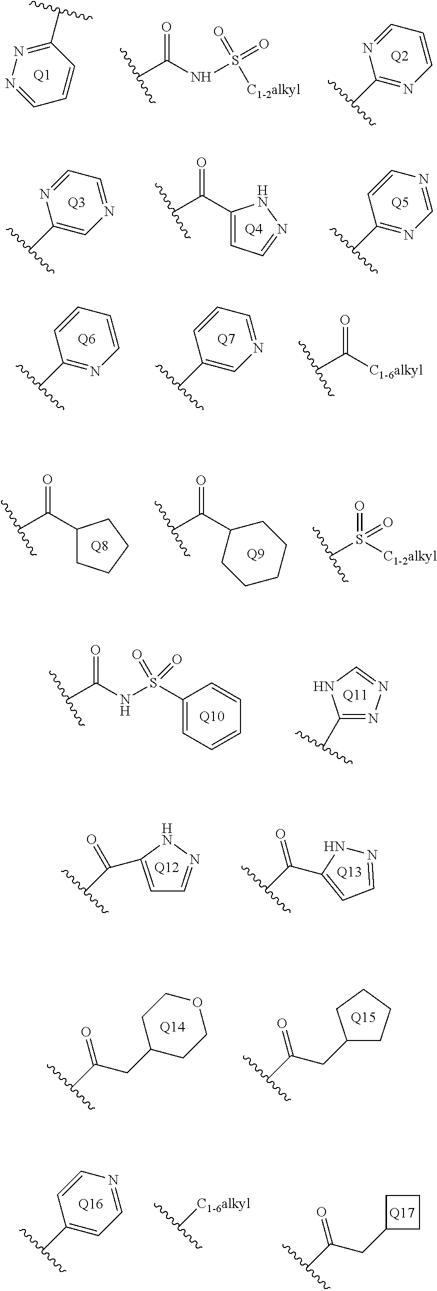 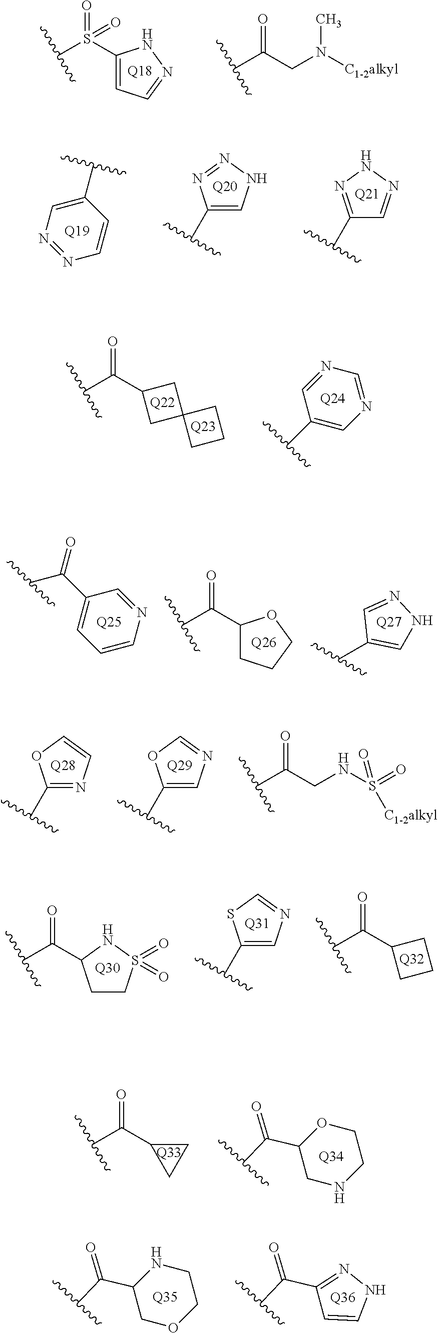 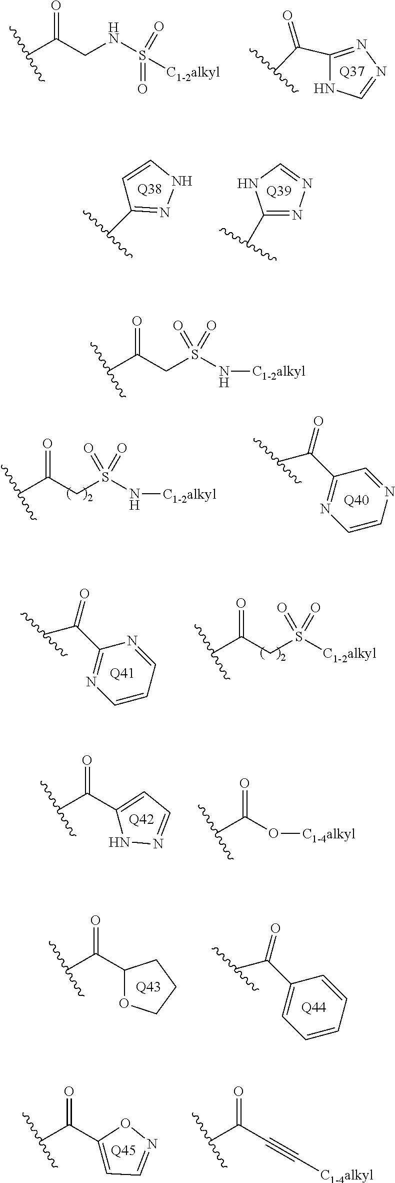 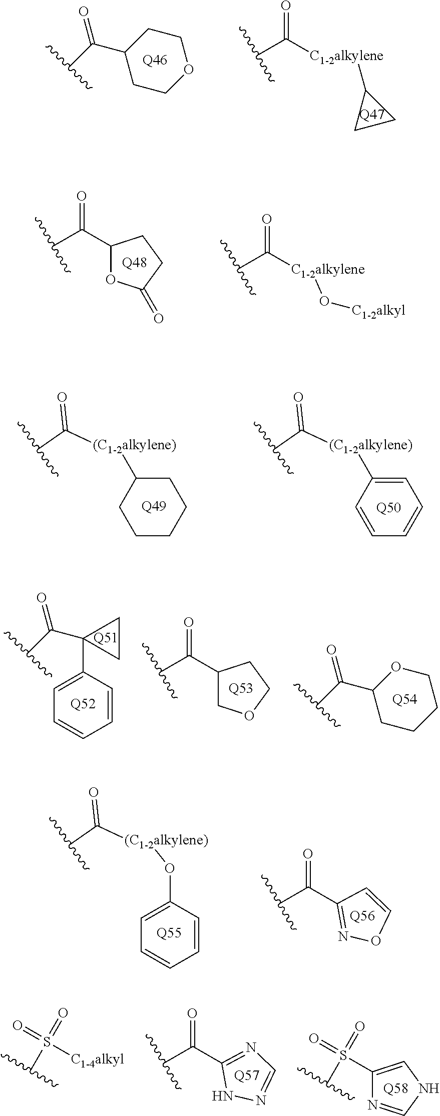 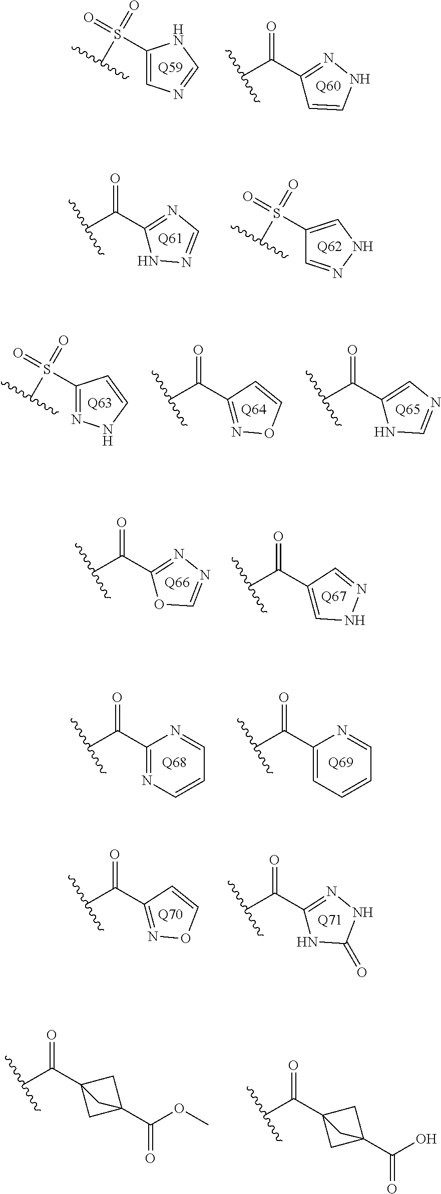
wherein each of Rings Q1-Q71 is optionally and independently substituted with 1-4 occurrences of J^{Y}, wherein J^{Y} is selected from —CN, halogen, —CH₃, —CF₃, —OH, —OCH₃, —NH₂, —NHCH₃, N(CH₃)₂, —C(O)OH, or —C(O)O(C₁₋₂ alkyl), and wherein each of said C₁₋₂alkyl, C₁₋₂alkylene, C₁₋₄alkyl, and C₁₋₆alkyl of X is optionally and independently substituted with 1-4 occurrences of J^{V} selected from —CN, halogen, —CH₃, —CF₃, —OH, —OCH₃, —NH₂, —NHCH₃, N(CH₃)₂, —C(O)OH, or —C(O)O(C₁₋₂ alkyl);
R⁴ is iso-propyl, tert-butyl, methylcyclopropyl, or cyanocyclopropyl; and
each J^{Y1}, J^{Y2} and J^{Y3} is independently H, halogen, CN, C₁₋₄alkyl, haloC₁₋₄alkyl, or OH.

32. The compound of claim 31, wherein X

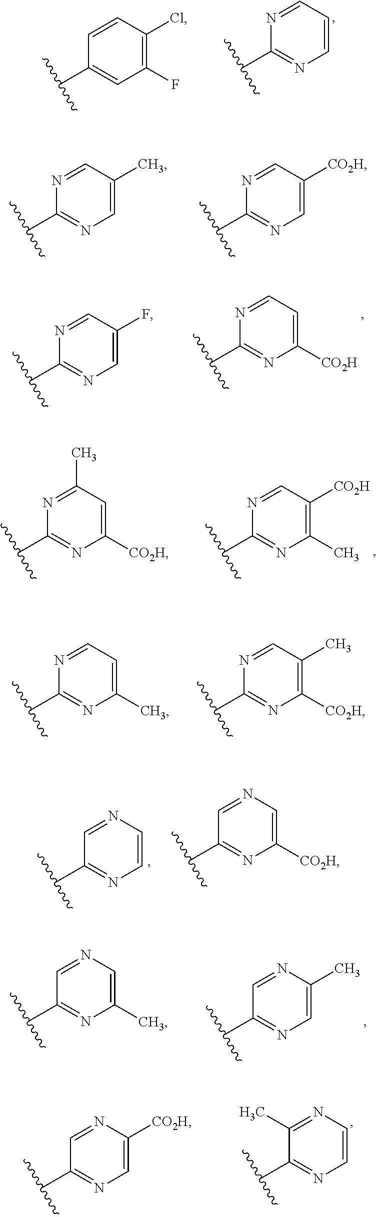

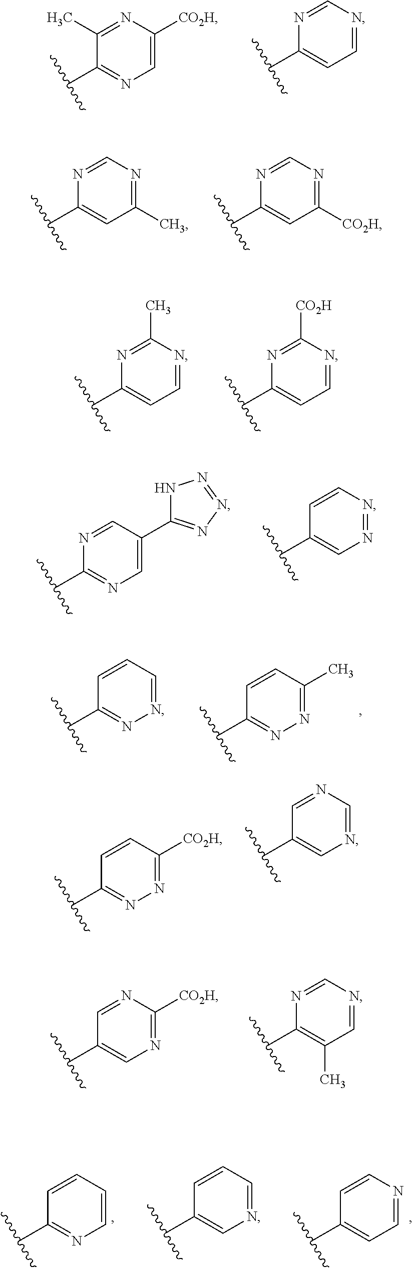

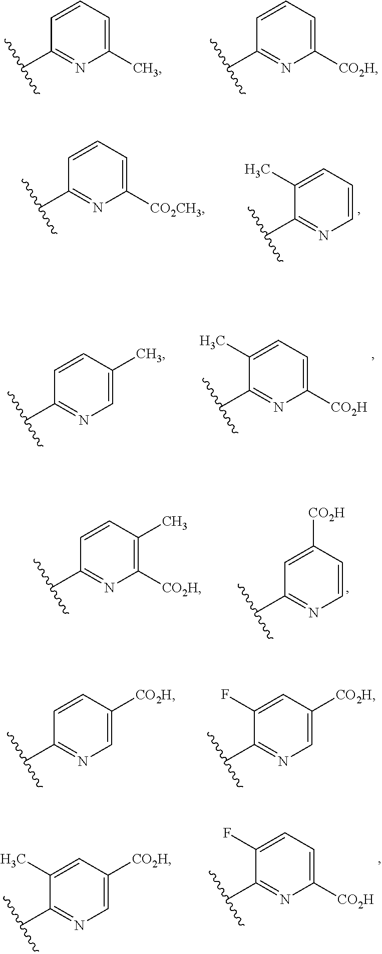

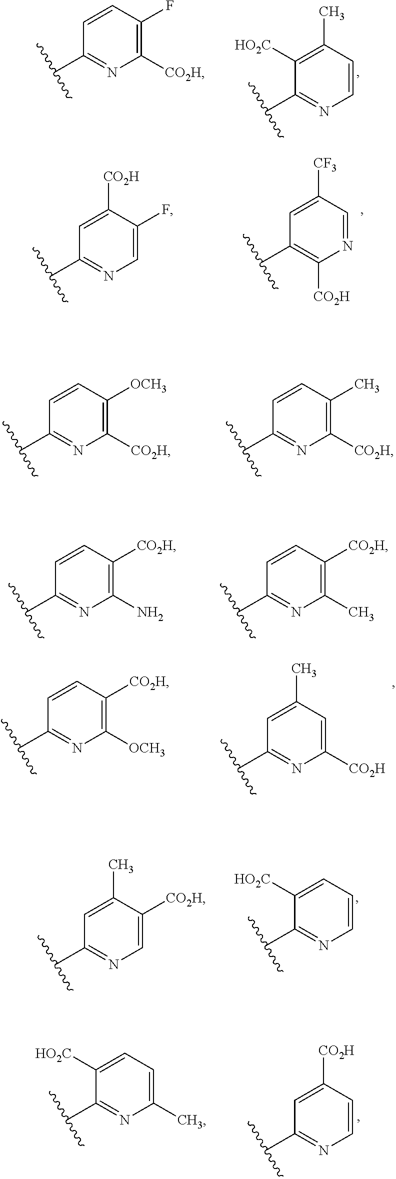

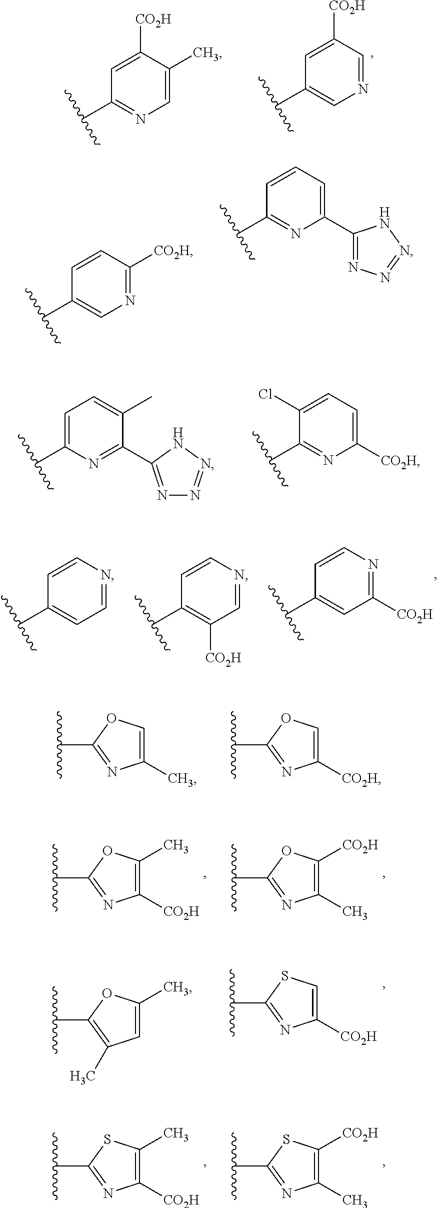

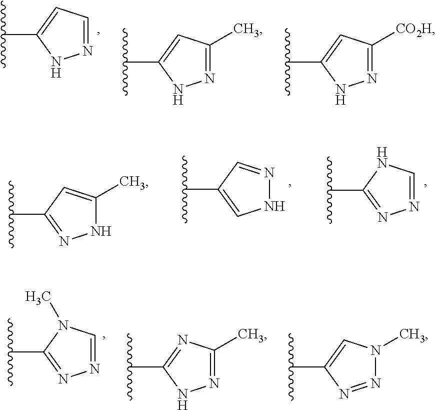

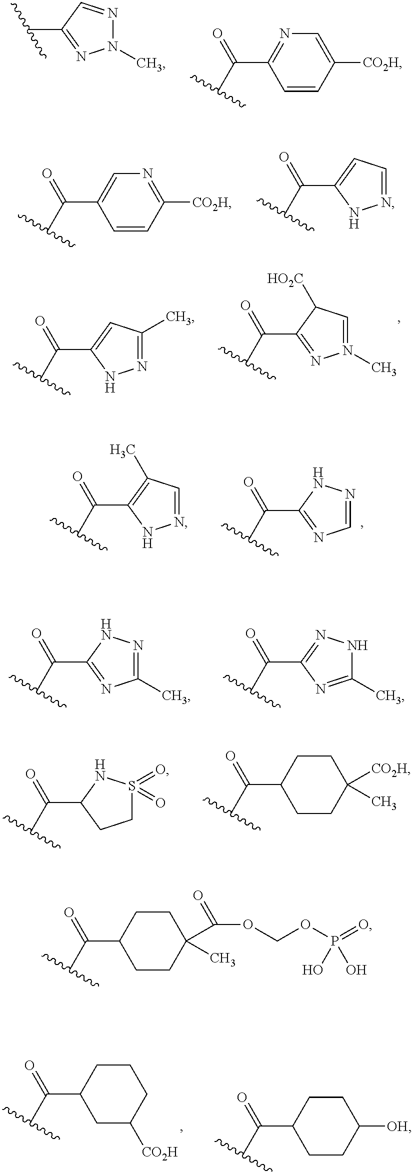

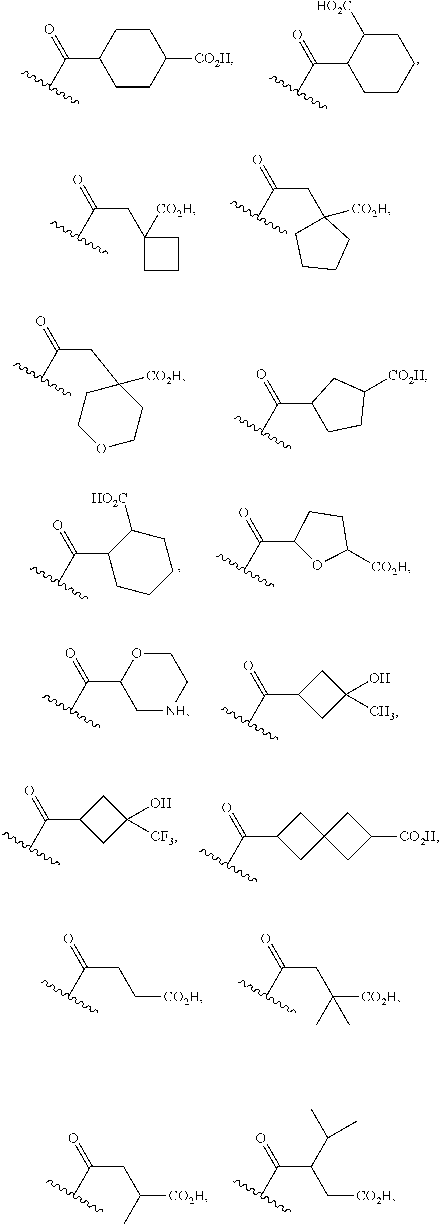

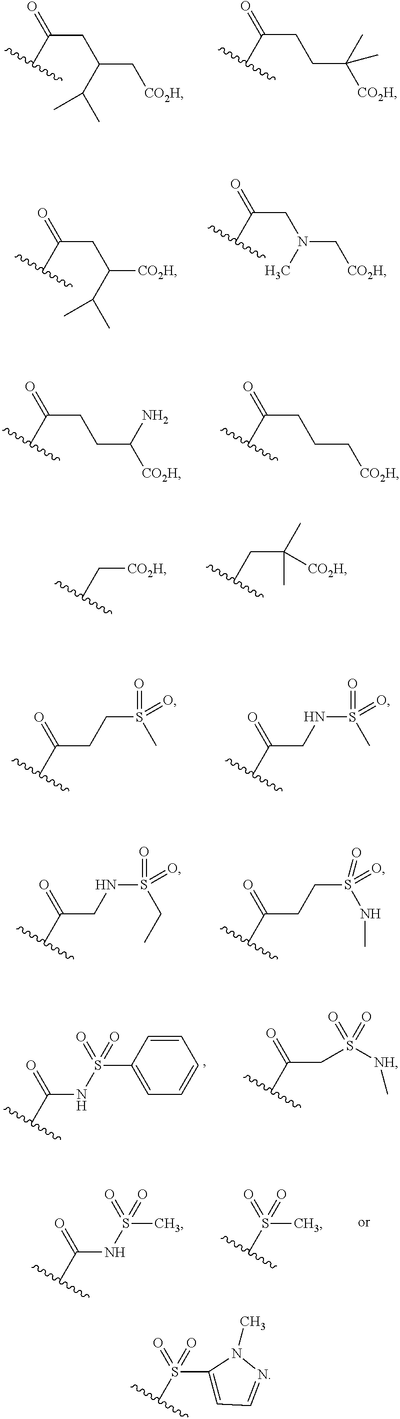

33. The compound of claim 31, wherein Ring A is

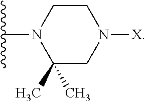

34. The compound of claim 33, where:
R⁴ is iso-propyl, tert-butyl, methylcyclopropyl, or C(CH₃)₂CH₂OCH₃;
X is 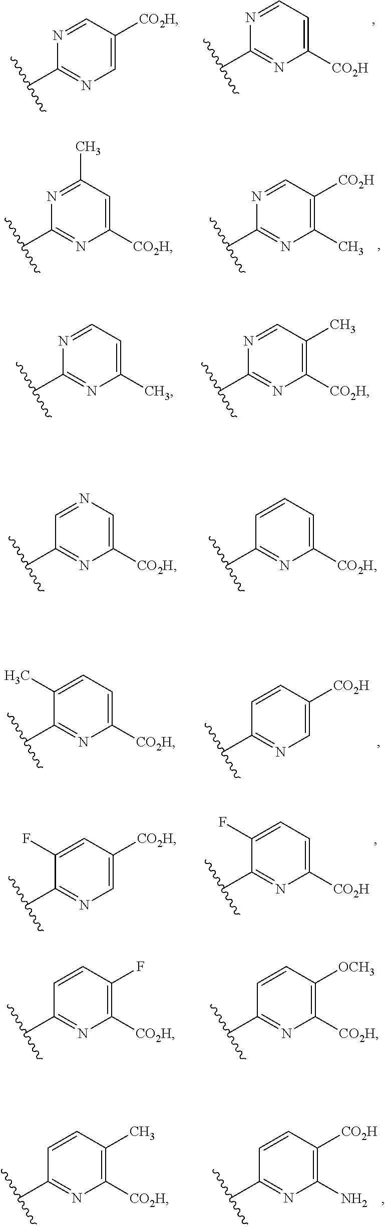 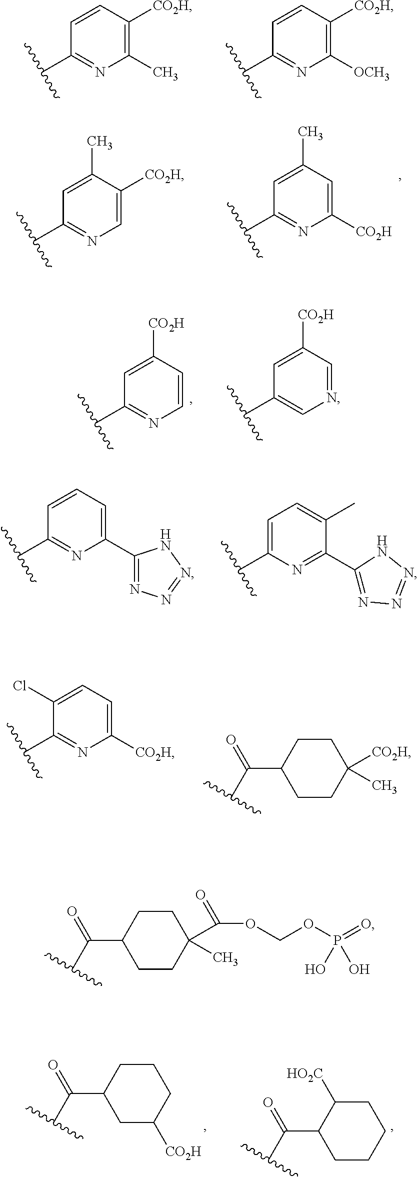 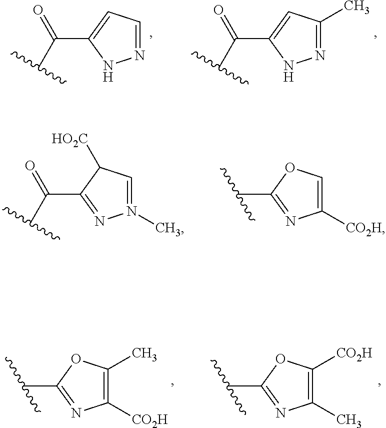 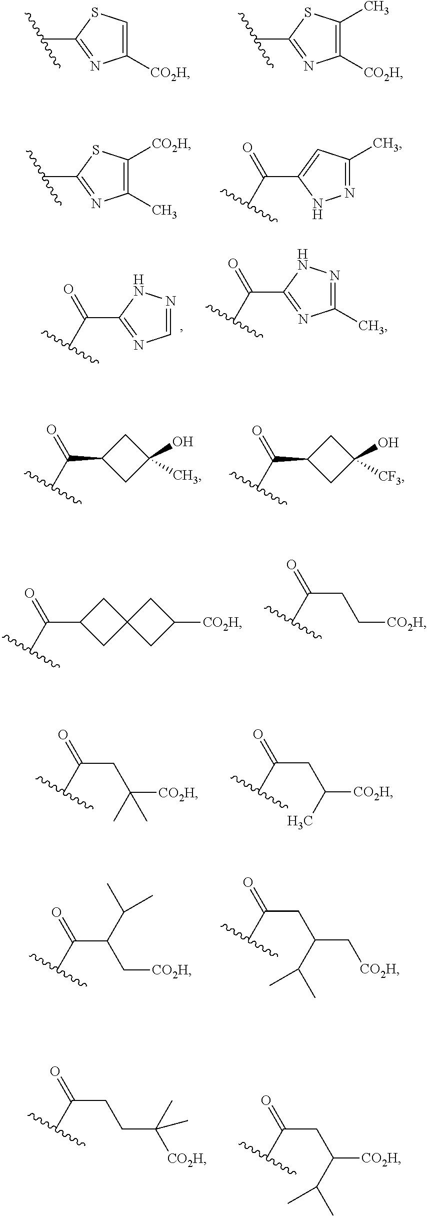 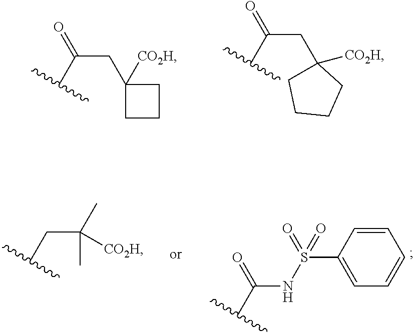
and
each J^{Y1}, J^{Y2}, and J^{Y3} is independently H, halogen, C₁₋₄alkyl, or haloC₁₋₄alkyl.

35. The compound of claim 29, wherein:
Ring A is 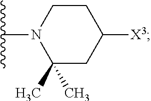
X³ is —CR₂—C(O)OR⁶, —N(R)R⁶, —CR₂—C(O)N(R)R⁶ or —CR₂—C(O)N(R)S(O)₂R⁶;
each R⁶ is independently —H or C₁₋₆ alkyl optionally substituted with with 1-4 occurrences of substituents selected from CN, halogen, C₁₋₄alkyl, haloC₁₋₄alkyl, —OH, —O(C₁₋₄alkyl), NH(C₁₋₄alkyl), N(C₁₋₄alkyl)₂, S(O)₂(C₁₋₄alkyl), NH₂, (CH₂)ₜ—C(O)OH, (CH₂)ₜ—C(O)O(C₁₋₂ alkyl), (CH(C₁₋₂ alkyl))ₜ—C(O)OH, (CH(C₁₋₂ alkyl))ₜ—C(O)O(C₁₋₂ alkyl), (C(C₁₋₂ alkyl)₂)ₜ—C(O)OH, or (C(C₁₋₂ alkyl)₂)ₜ—C(O)O(C₁₋₂ alkyl), wherein each t is independently 0 or 1.

36. The compound of claim 35, wherein:
R⁴ is iso-propyl, tert-butyl, methylcyclopropyl, or C(CH₃)₂CH₂OCH₃;
X³ is —CH₂—C(O)OH, —NH₂, —NH(C₁₋₂ alkyl), —CH₂—C(O)NH—C₁₋₂ alkyl, —CH₂—C(O)NHS(O)₂C₁₋₂ alkyl, or —CH₂—C(O)NH—CN, wherein each of said C₁₋₂ alkyl is optionally and independently substituted with 1-4 occurrences of substitutents selected from CN, halogen, —OH, —OCH₃, or —C(O)OH;
each J^{Y1}, J^{Y2}, and J^{Y3} is independently H, halogen, C₁₋₄alkyl, or haloC₁₋₄alkyl.

37. The compound of claim 2, represented by Structural Formula II or III, or a pharmaceutically acceptable salt thereof: 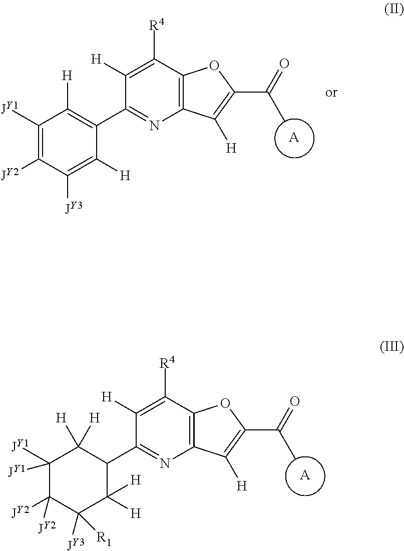
wherein each of J^{Y1}, J^{Y2}, and J^{Y3} is independently as defined for J^{Y}, and J^{Y}, R⁴, and Ring A are independently as defined in claim 2.

38. The compound of claim 37, wherein
Ring A is 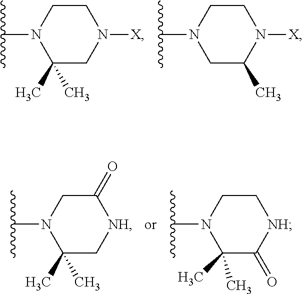
R⁴ is iso-propyl, tert-butyl, methylcyclopropyl, or cyanocyclopropyl:
each X is independently R⁵ or —C(O)R⁵;
each R⁵ is independently (V)ₐ—Y; wherein
V is C₁₋₆aliphatic wherein up to two carbon units of said C₁₋₆aliphatic can each be optionally and independently replaced with —O— or —C(O)—; wherein V is optionally substituted with 1-3 occurrences of halogen, CN, C₁₋₄alkyl, OH, O(C₁₋₄alkyl), NH₂, or —NHC(O)C₁₋₄alkyl; and
Y is H, a 3-6 membered cycloalkyl, isoxazolyl, oxadiazolyl, pyrazolyl, imidazolyl, triazolyl, thienyl, pyrazolyl, tetrahydrofuranyl, tetrahydropyranyl, pyridinyl, pyrimidinyl, or phenyl; wherein Y is optionally substituted with 1-4 occurrences of J^{Y}; and
each J^{Y1}, J^{Y2}, and J^{Y3} is independently H, halogen, CN, C₁₋₄alkyl, haloC₁₋₄alkyl, or OH.

39. The compound of claim 37, wherein:
Ring A is 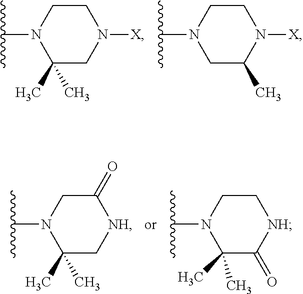
R⁴ is iso-propyl, tert-butyl, methylcyclopropyl, or cyanocyclopropyl;
each J^{Y1}, J^{Y2}, and J^{Y3} is independently H, halogen, CN, C₁₋₄alkyl, haloC₁₋₄alkyl, or OH;
each X is independently selected from the group consisting of —C(O)CH(CH₃)OH, —C(O)OC(CH₃)₃, —C(O)C(CH₃)₂OH, —C(O)C(OH)(cyclobutyl), —C(O)CH₂CN, —C(O)tetrahydrofuranyl, —C(O)phenyl, —C(O)isoxazolyl, —C(O)CH(OH)CH(CH₃)₂, —C(O)CH(CH₃)₃, —C(O)CH(OH)CH₃, —C(O)C(CH₃)₂F, —C(O)CH₂OCH₃, —C(O)CH(OH)CH₂C(CH₃)₃, —C(O)methylcyclopropyl, —C(O)dimethylcyclopropyl, —C(O)gem dimethylcyclopropyl, —C(O)C═CCH₃, —C(O)CH₂C(CH₃)₃, —C(O)hydroxyl-tetrahydropyranyl, —C(O)CH₃, —C(O)CH(OH)CH₂CH₃, —C(O)CH(OCH₃)CH₃, —C(O)tetrahydrofuranyl, —C(O)CH(OH)(cyclopropyl), 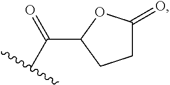 [SMILES: *C(=O)C1CCC(=O)O1]
.
C(O)CH(OH)(CH₂)₃CH₃, —C(O)C(CH₃)(CH₂CH₃)OH, —C(O)CH(OH)CH₂CH(CH₃)₂, —C(O)CH₂OCH₂CH₂OCH₃, —C(O)CH(OH)CH₂(CH₃)₃, —C(O)C(CH₃)(CF₃)OH, —CH(OH)(cyclohexyl), —CH(OH)CH₂phenyl, —C(O)cyclohexyl, —C(O)(methylcyclohexyl), —C(O)CH(CH₃)CH₂CH₃, —C(O)CH(phenyl)CH₂CH₃, —C(O)cyclobutyl, —C(O)(cyclopropyl), —C(O)(tetramethylcyclopropyl), —C(O)(cyanocyclopropyl), —C(O)C(CH₃)₂CH₂CH₃, 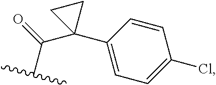 [SMILES: *C(=O)C1(c2ccc(Cl)cc2)CC1]
—C(O)(tetrahydropyranyl), —C(O)(CF₃-cyclopropyl), —C(O)(hydroxycyclobutyl), —C(O)(hydroxytetrahydropyranyl), 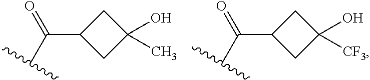
—C(O)CH₂C(CH₃)₂OH, —C(O)pyrazolyl, —C(O)CH(OCH₃)(phenyl), —C(O)CH(CH₃)(phenyl), —C(O)C(CF₃)(OH)CH₃, —C(O)(OH)CH₂CH₃)₂, 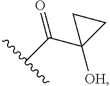 [SMILES: *C(=O)C1(O)CC1]
—C(O)cyclopentyl, —C(O)(difluorocyclopropyl), —C(O)isoxazolyl, —C(O)C(CH₃)₃, —C(O)CH(OH)(4-fluorphenyl), —C(O)OCH₂CH₃, —C(CH₃)₂CH₂OCH₃, and —C(O)dimethylpyrazolyl.

40. The compound of claim 37, wherein:
Ring A is 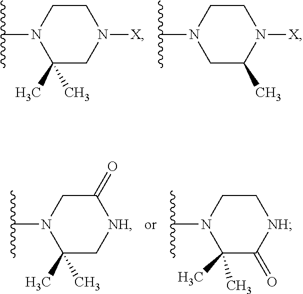
R⁴ is iso-propyl, tert-butyl, methylcyclopropyl, or cyanocyclopropyl;
each J^{Y1}, J^{Y2}, and J^{Y3} is independently H, halogen, CN, C₁₋₄alkyl, haloC₁₋₄alkyl, or OH: each X is independently selected from the group consisting of

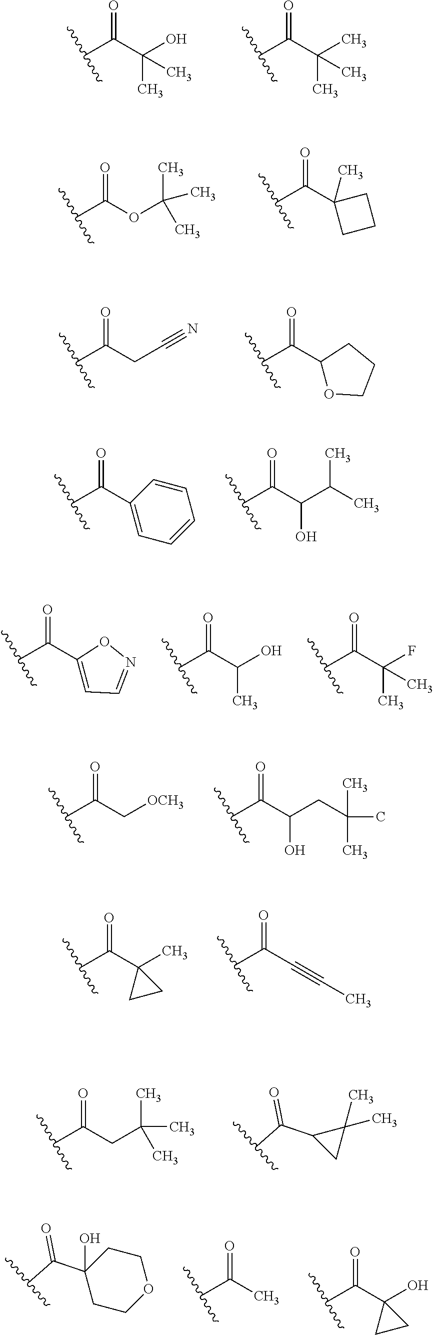

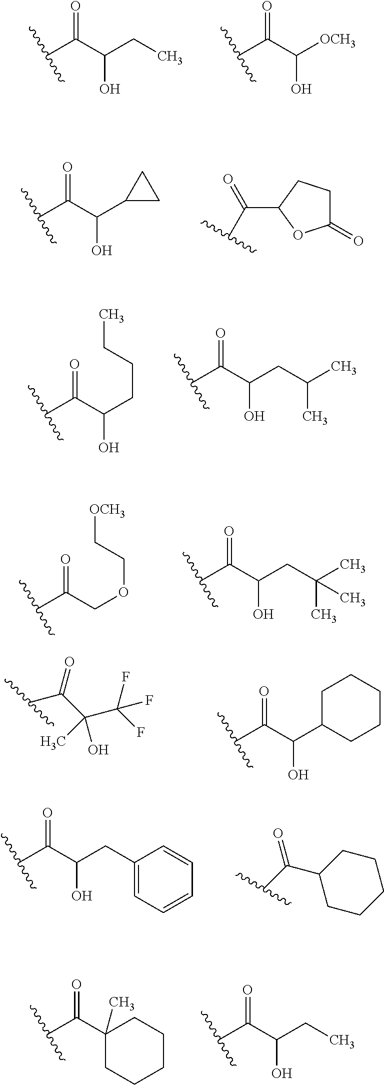

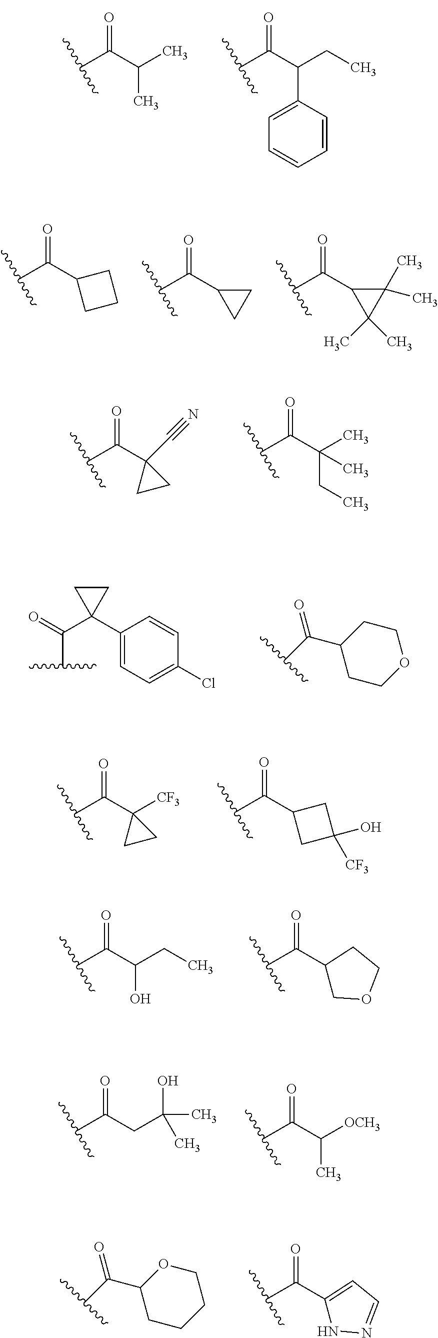

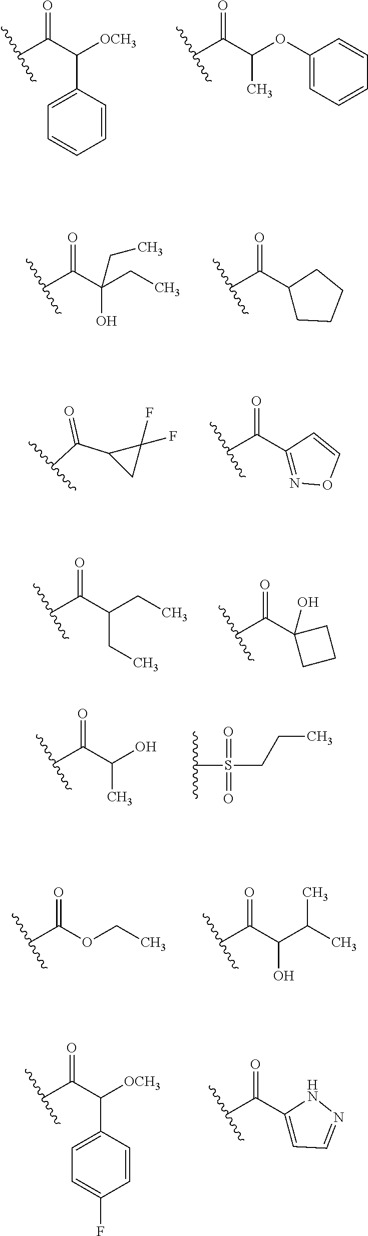

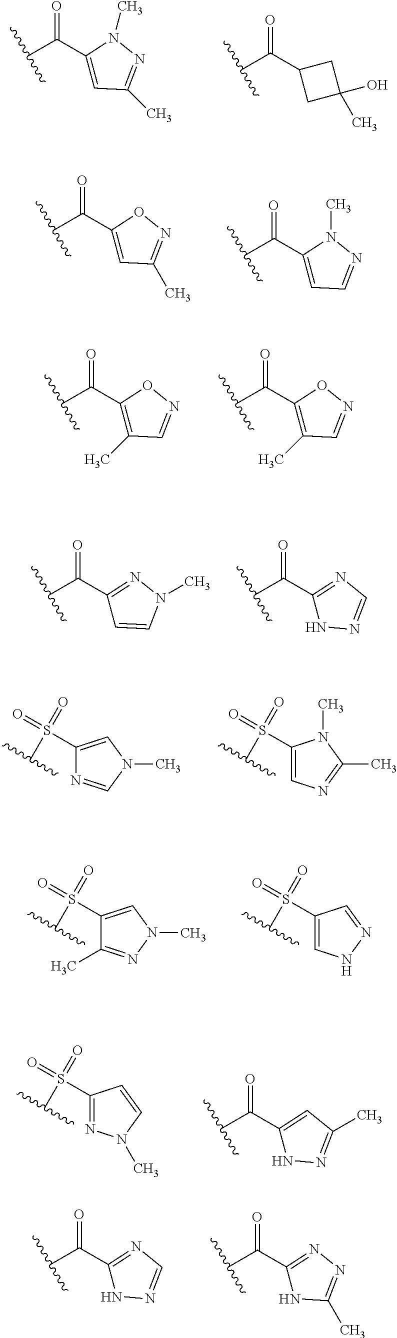

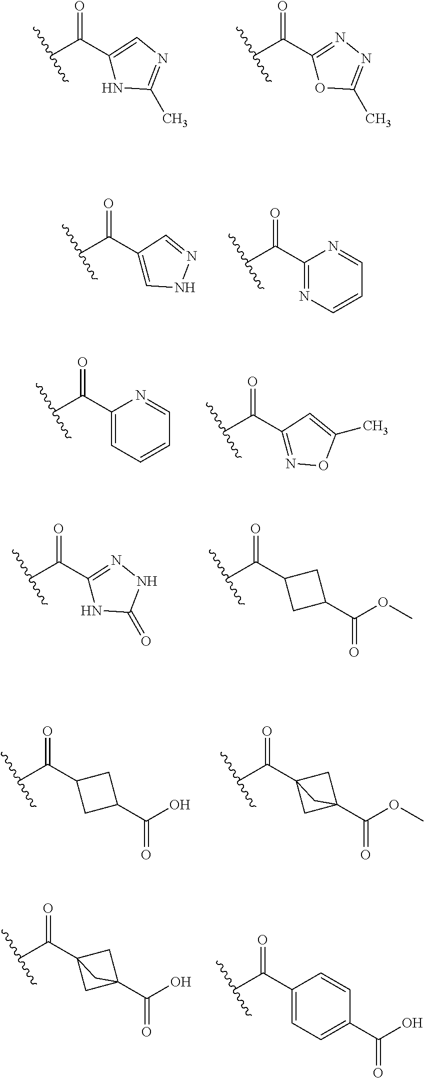

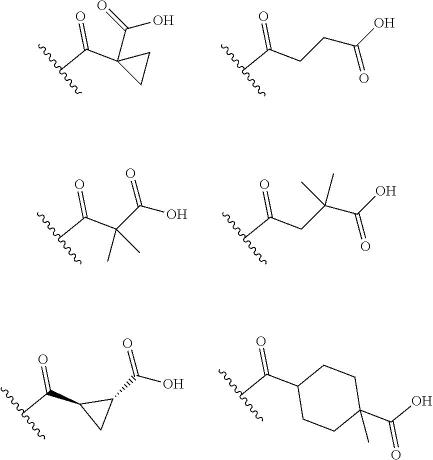

41. The compound of any one of claims 29-40, wherein each of J^{Y1}, J^{Y2} and J^{Y3} is independently H, Cl, F, CH₃, or CF₃.

42. The compound of claim 37, represented by Structural Formula (II) or a pharmaceutically acceptable salt thereof.

43. The compound of claim 42, wherein
Ring A is 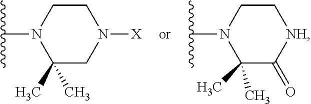
R⁴ is iso-propyl, tert-butyl, methylcyclopropyl, or C(CH₃)₂CH₂OCH₃;
X is 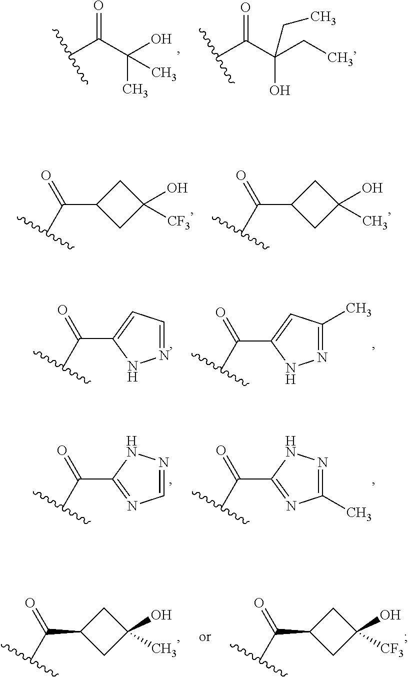
and each J^{Y1}, J^{Y2}, and J^{Y3} is independently H, halogen, C₁₋₄alkyl, or haloC₁₋₄alkyl.

44. The compound of claim 43, wherein J^{Y1} is H, F, or CH₃; J^{Y3} is H; J^{Y2} is Cl, F, CH₃, or CF₃.

45. The compound of any one of claims 42-44, wherein Ring A is

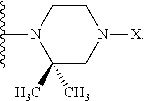

46. The compound of claim 45, wherein X is

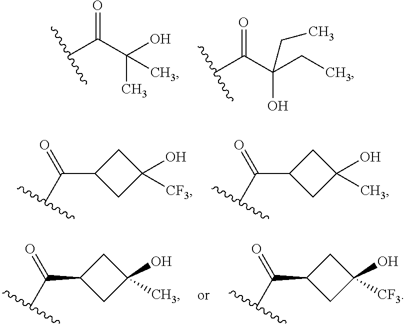

47. The compound of claim 45, wherein X is

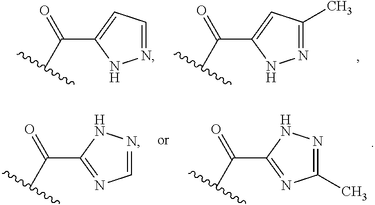

48. The compound of any one of claims 42-44, wherein Ring A is

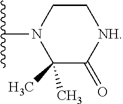

49. The compound of claim 37, represented by Structural Formula (III) or a pharmaceutically acceptable salt thereof.

50. The compound of claim 49, wherein
Ring A is 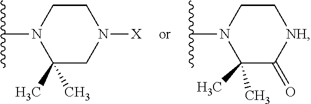
R⁴ is iso-propyl, tert-butyl, methylcyclopropyl, or C(CH₃)₂CH₂OCH₃;
X is 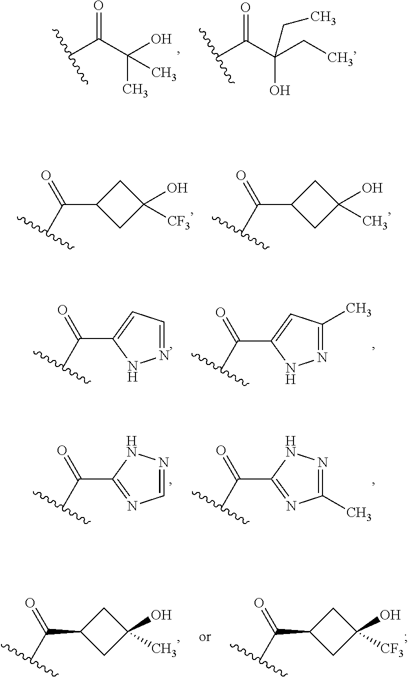
and
each J^{Y1}, J^{Y2}, and J^{Y3} is independently H, halogen, C₁₋₄alkyl, or haloC₁₋₄alkyl.

51. The compound of claim 50, wherein each of J^{Y1}, J^{Y2} and J^{Y3} is independently H, Cl, F, CH₃, or CF₃.

52. The compound of claim 50 or 51, wherein Ring A is

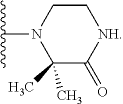

53. A compound represented by any one of the following chemical structures or a pharmaceutically acceptable salt thereof:

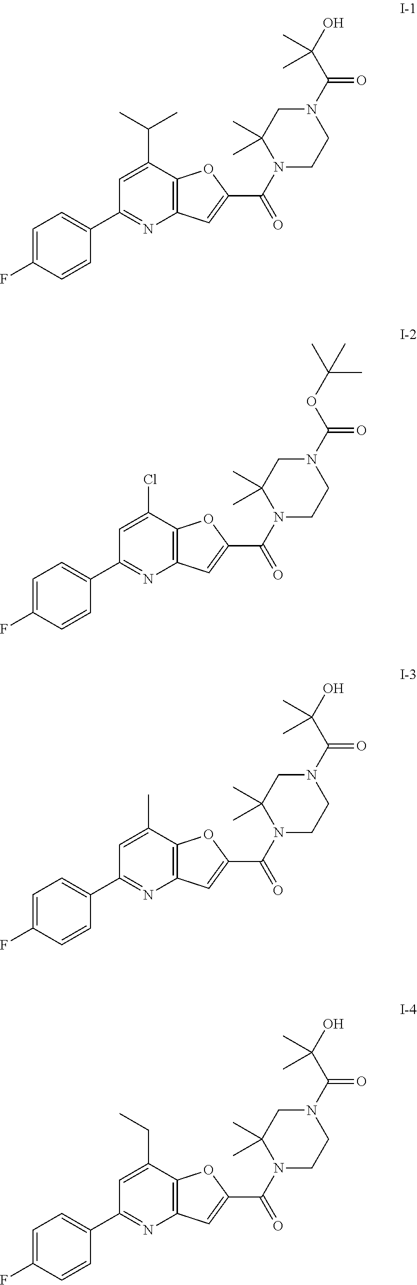 [SMILES: CC(C)c1cc(-c2ccc(F)cc2)nc2cc(C(=O)N3CCN(C(=O)C(C)(C)O)CC3(C)C)oc12 ; CC(C)(C)OC(=O)N1CCN(C(=O)c2cc3nc(-c4ccc(F)cc4)cc(Cl)c3o2)C(C)(C)C1 ; Cc1cc(-c2ccc(F)cc2)nc2cc(C(=O)N3CCN(C(=O)C(C)(C)O)CC3(C)C)oc12 ; CCc1cc(-c2ccc(F)cc2)nc2cc(C(=O)N3CCN(C(=O)C(C)(C)O)CC3(C)C)oc12]

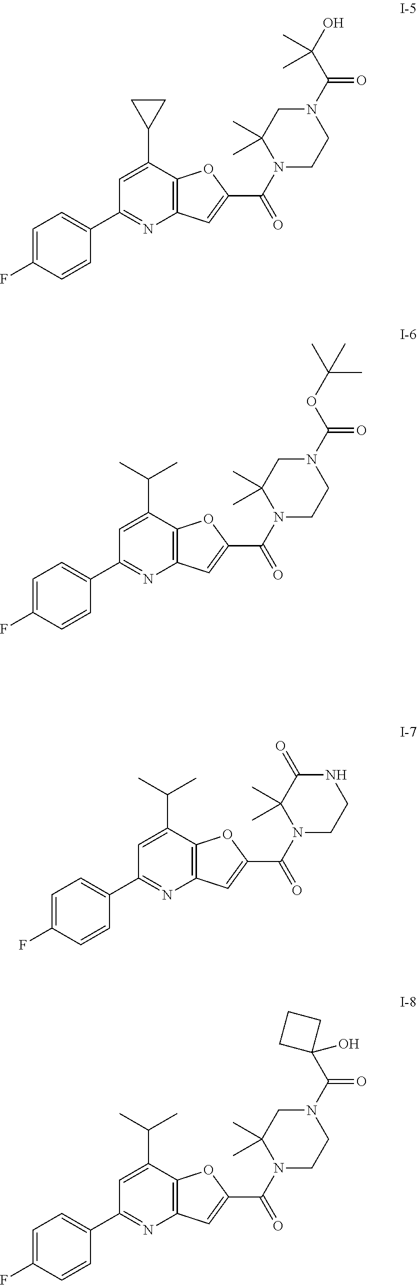 [SMILES: CC(C)(O)C(=O)N1CCN(C(=O)c2cc3nc(-c4ccc(F)cc4)cc(C4CC4)c3o2)C(C)(C)C1 ; CC(C)c1cc(-c2ccc(F)cc2)nc2cc(C(=O)N3CCN(C(=O)OC(C)(C)C)CC3(C)C)oc12 ; CC(C)c1cc(-c2ccc(F)cc2)nc2cc(C(=O)N3CCNC(=O)C3(C)C)oc12 ; CC(C)c1cc(-c2ccc(F)cc2)nc2cc(C(=O)N3CCN(C(=O)C4(O)CCC4)CC3(C)C)oc12]

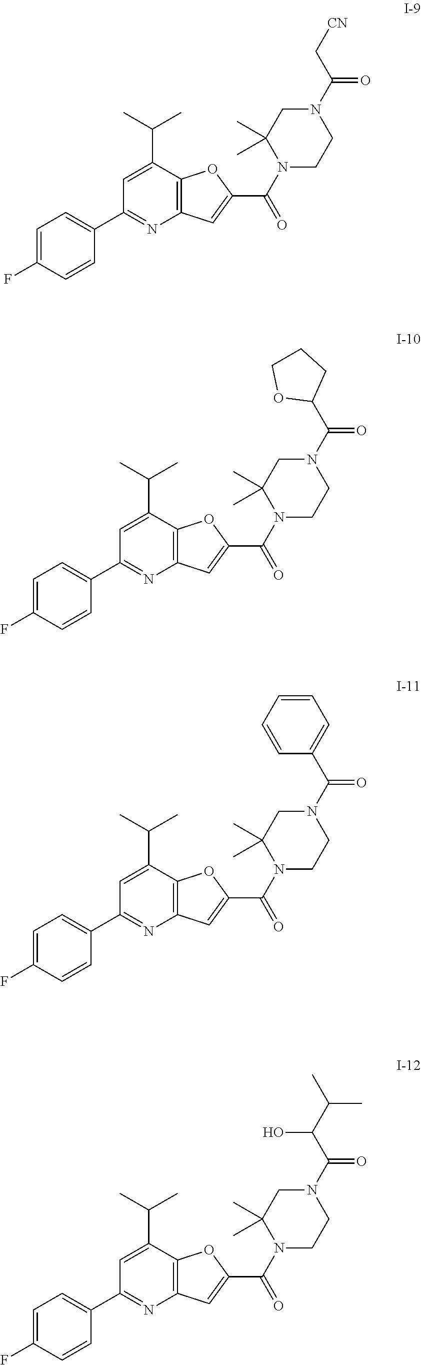 [SMILES: CC(C)c1cc(-c2ccc(F)cc2)nc2cc(C(=O)N3CCN(C(=O)CC#N)CC3(C)C)oc12 ; CC(C)c1cc(-c2ccc(F)cc2)nc2cc(C(=O)N3CCN(C(=O)C4CCCO4)CC3(C)C)oc12 ; CC(C)c1cc(-c2ccc(F)cc2)nc2cc(C(=O)N3CCN(C(=O)c4ccccc4)CC3(C)C)oc12 ; CC(C)c1cc(-c2ccc(F)cc2)nc2cc(C(=O)N3CCN(C(=O)C(O)C(C)C)CC3(C)C)oc12]

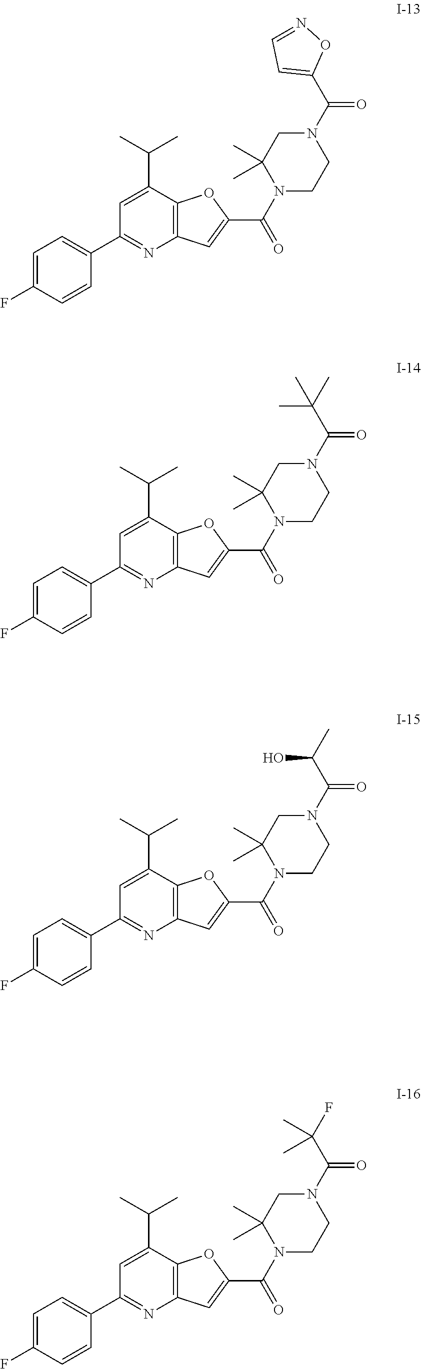 [SMILES: CC(C)c1cc(-c2ccc(F)cc2)nc2cc(C(=O)N3CCN(C(=O)c4ccno4)CC3(C)C)oc12 ; CC(C)c1cc(-c2ccc(F)cc2)nc2cc(C(=O)N3CCN(C(=O)C(C)(C)C)CC3(C)C)oc12 ; CC(C)c1cc(-c2ccc(F)cc2)nc2cc(C(=O)N3CCN(C(=O)[C@H](C)O)CC3(C)C)oc12 ; CC(C)c1cc(-c2ccc(F)cc2)nc2cc(C(=O)N3CCN(C(=O)C(C)(C)F)CC3(C)C)oc12]

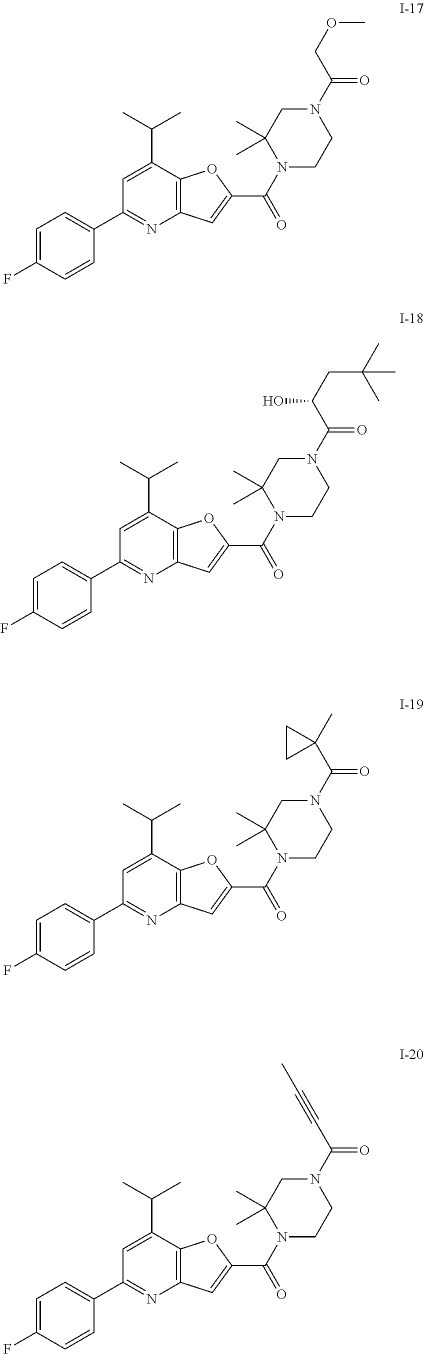 [SMILES: COCC(=O)N1CCN(C(=O)c2cc3nc(-c4ccc(F)cc4)cc(C(C)C)c3o2)C(C)(C)C1 ; CC(C)c1cc(-c2ccc(F)cc2)nc2cc(C(=O)N3CCN(C(=O)[C@H](O)CC(C)(C)C)CC3(C)C)oc12 ; CC(C)c1cc(-c2ccc(F)cc2)nc2cc(C(=O)N3CCN(C(=O)C4(C)CC4)CC3(C)C)oc12 ; CC#CC(=O)N1CCN(C(=O)c2cc3nc(-c4ccc(F)cc4)cc(C(C)C)c3o2)C(C)(C)C1]

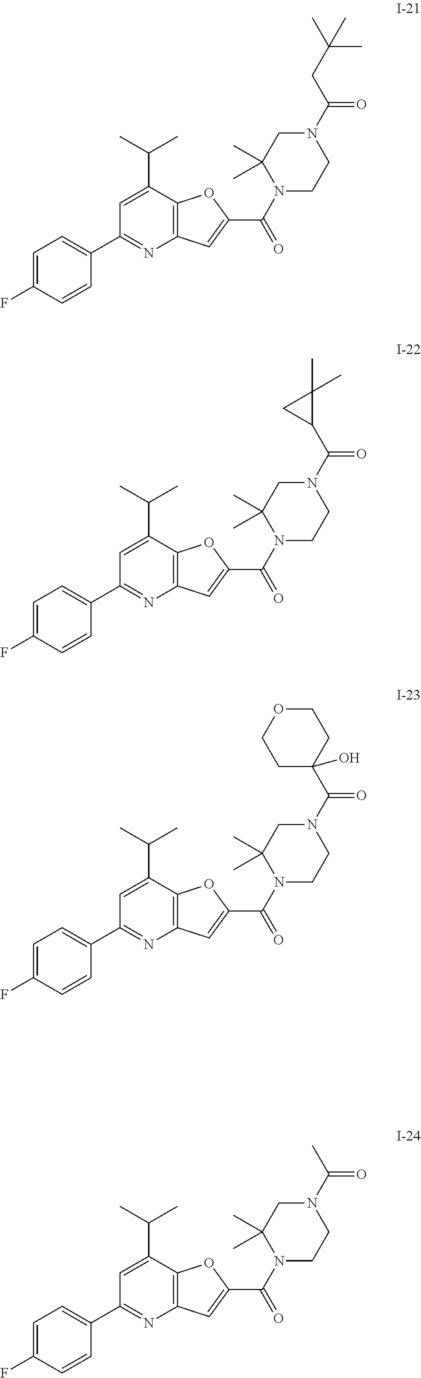 [SMILES: CC(C)c1cc(-c2ccc(F)cc2)nc2cc(C(=O)N3CCN(C(=O)CC(C)(C)C)CC3(C)C)oc12 ; CC(C)c1cc(-c2ccc(F)cc2)nc2cc(C(=O)N3CCN(C(=O)C4CC4(C)C)CC3(C)C)oc12 ; CC(C)c1cc(-c2ccc(F)cc2)nc2cc(C(=O)N3CCN(C(=O)C4(O)CCOCC4)CC3(C)C)oc12 ; CC(=O)N1CCN(C(=O)c2cc3nc(-c4ccc(F)cc4)cc(C(C)C)c3o2)C(C)(C)C1]

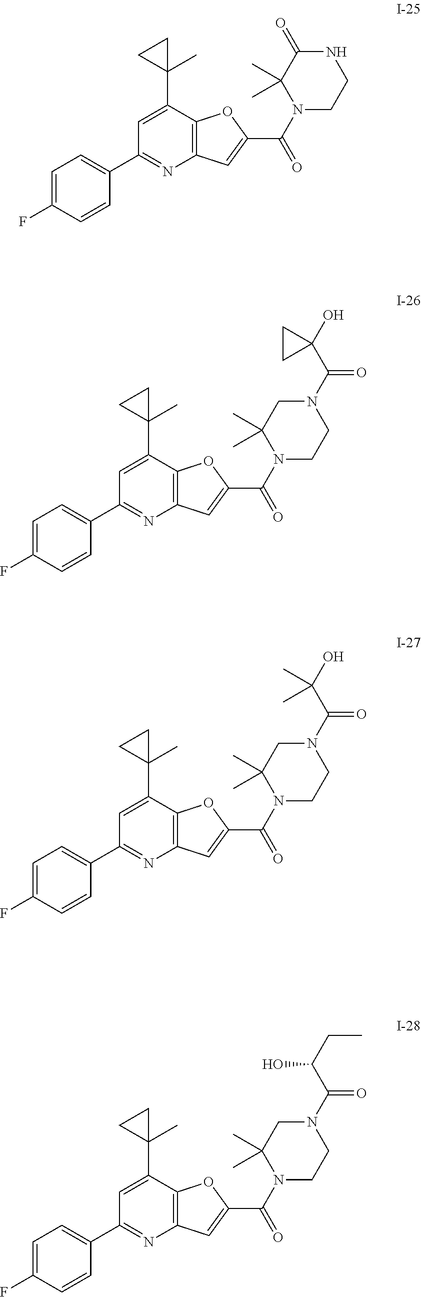 [SMILES: CC1(c2cc(-c3ccc(F)cc3)nc3cc(C(=O)N4CCNC(=O)C4(C)C)oc23)CC1 ; CC1(c2cc(-c3ccc(F)cc3)nc3cc(C(=O)N4CCN(C(=O)C5(O)CC5)CC4(C)C)oc23)CC1 ; CC(C)(O)C(=O)N1CCN(C(=O)c2cc3nc(-c4ccc(F)cc4)cc(C4(C)CC4)c3o2)C(C)(C)C1 ; CC[C@@H](O)C(=O)N1CCN(C(=O)c2cc3nc(-c4ccc(F)cc4)cc(C4(C)CC4)c3o2)C(C)(C)C1]

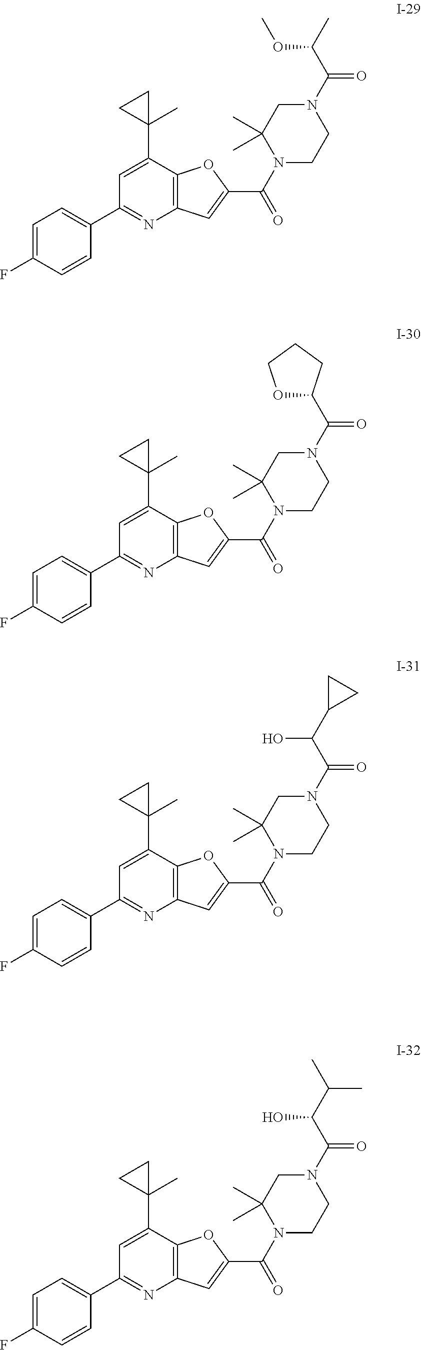 [SMILES: CO[C@H](C)C(=O)N1CCN(C(=O)c2cc3nc(-c4ccc(F)cc4)cc(C4(C)CC4)c3o2)C(C)(C)C1 ; CC1(c2cc(-c3ccc(F)cc3)nc3cc(C(=O)N4CCN(C(=O)[C@H]5CCCO5)CC4(C)C)oc23)CC1 ; CC1(c2cc(-c3ccc(F)cc3)nc3cc(C(=O)N4CCN(C(=O)C(O)C5CC5)CC4(C)C)oc23)CC1 ; CC(C)[C@@H](O)C(=O)N1CCN(C(=O)c2cc3nc(-c4ccc(F)cc4)cc(C4(C)CC4)c3o2)C(C)(C)C1]

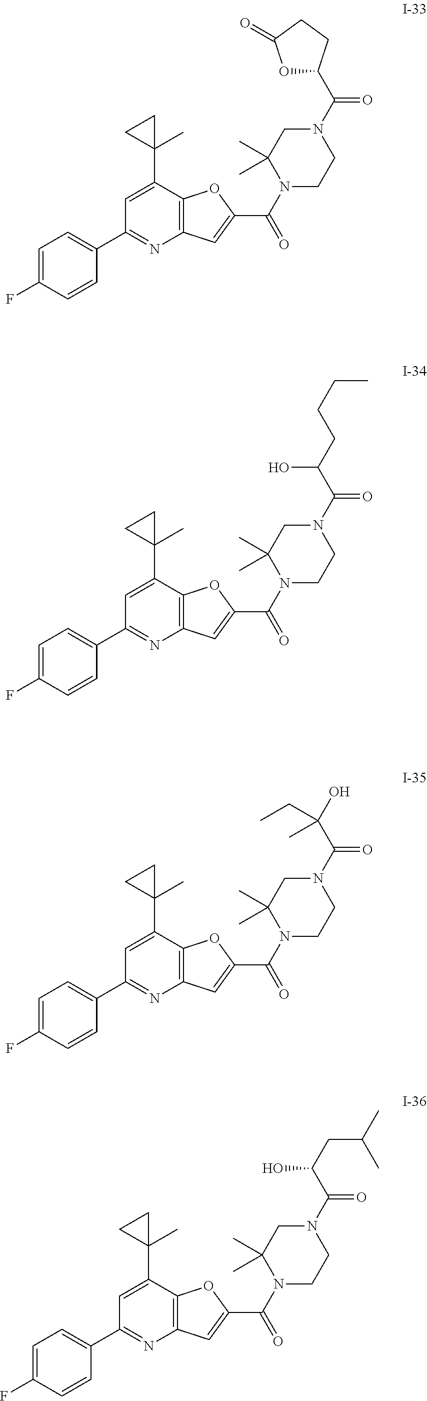 [SMILES: CC1(c2cc(-c3ccc(F)cc3)nc3cc(C(=O)N4CCN(C(=O)[C@H]5CCC(=O)O5)CC4(C)C)oc23)CC1 ; CCCCC(O)C(=O)N1CCN(C(=O)c2cc3nc(-c4ccc(F)cc4)cc(C4(C)CC4)c3o2)C(C)(C)C1 ; CCC(C)(O)C(=O)N1CCN(C(=O)c2cc3nc(-c4ccc(F)cc4)cc(C4(C)CC4)c3o2)C(C)(C)C1 ; CC(C)C[C@@H](O)C(=O)N1CCN(C(=O)c2cc3nc(-c4ccc(F)cc4)cc(C4(C)CC4)c3o2)C(C)(C)C1]

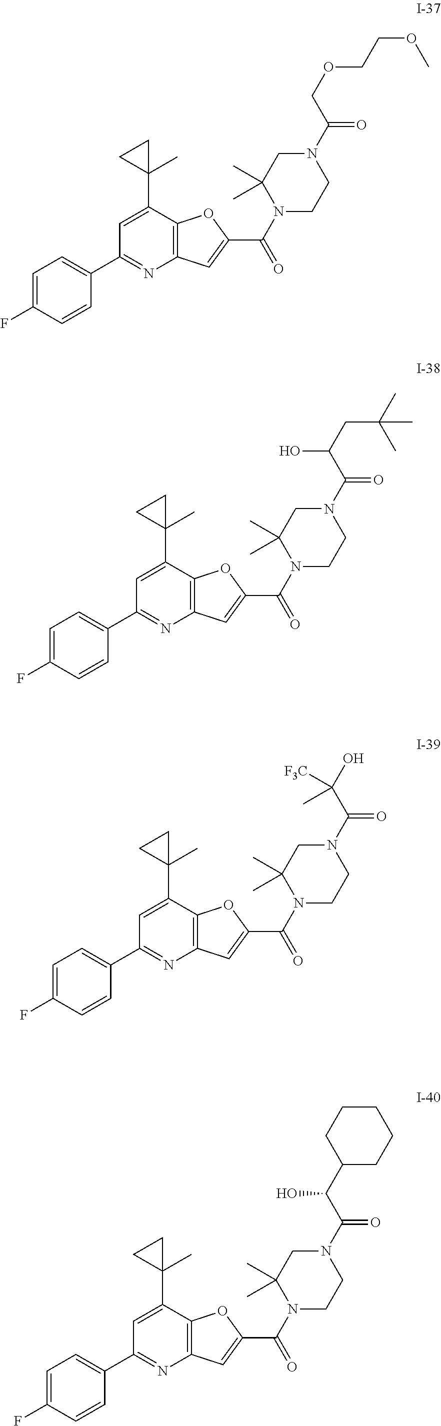 [SMILES: COCCOCC(=O)N1CCN(C(=O)c2cc3nc(-c4ccc(F)cc4)cc(C4(C)CC4)c3o2)C(C)(C)C1 ; CC(C)(C)CC(O)C(=O)N1CCN(C(=O)c2cc3nc(-c4ccc(F)cc4)cc(C4(C)CC4)c3o2)C(C)(C)C1 ; CC1(c2cc(-c3ccc(F)cc3)nc3cc(C(=O)N4CCN(C(=O)C(C)(O)C(F)(F)F)CC4(C)C)oc23)CC1 ; CC1(c2cc(-c3ccc(F)cc3)nc3cc(C(=O)N4CCN(C(=O)[C@H](O)C5CCCCC5)CC4(C)C)oc23)CC1]

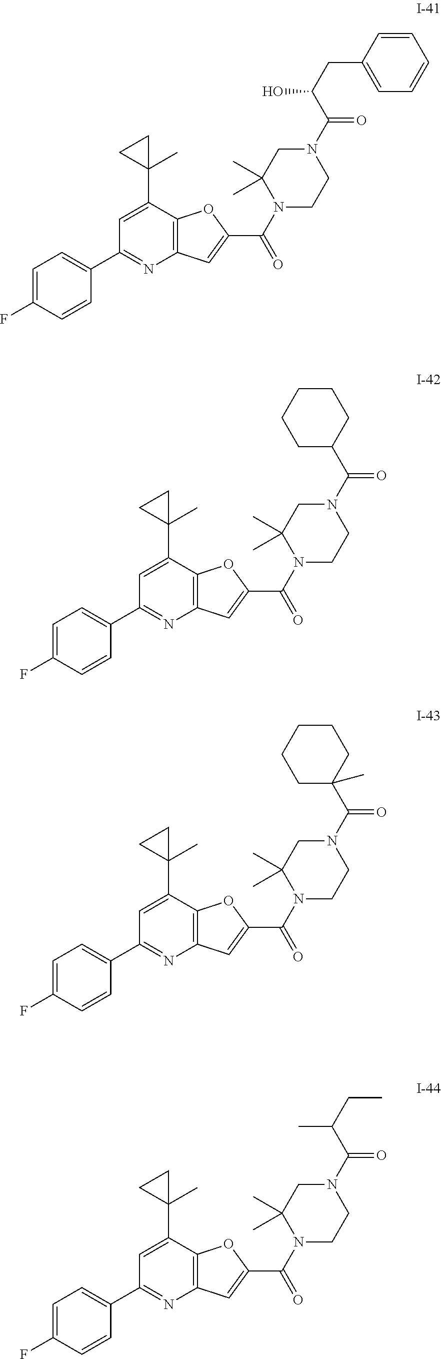 [SMILES: CC1(c2cc(-c3ccc(F)cc3)nc3cc(C(=O)N4CCN(C(=O)[C@H](O)Cc5ccccc5)CC4(C)C)oc23)CC1 ; CC1(c2cc(-c3ccc(F)cc3)nc3cc(C(=O)N4CCN(C(=O)C5CCCCC5)CC4(C)C)oc23)CC1 ; CC1(C(=O)N2CCN(C(=O)c3cc4nc(-c5ccc(F)cc5)cc(C5(C)CC5)c4o3)C(C)(C)C2)CCCCC1 ; CCC(C)C(=O)N1CCN(C(=O)c2cc3nc(-c4ccc(F)cc4)cc(C4(C)CC4)c3o2)C(C)(C)C1]

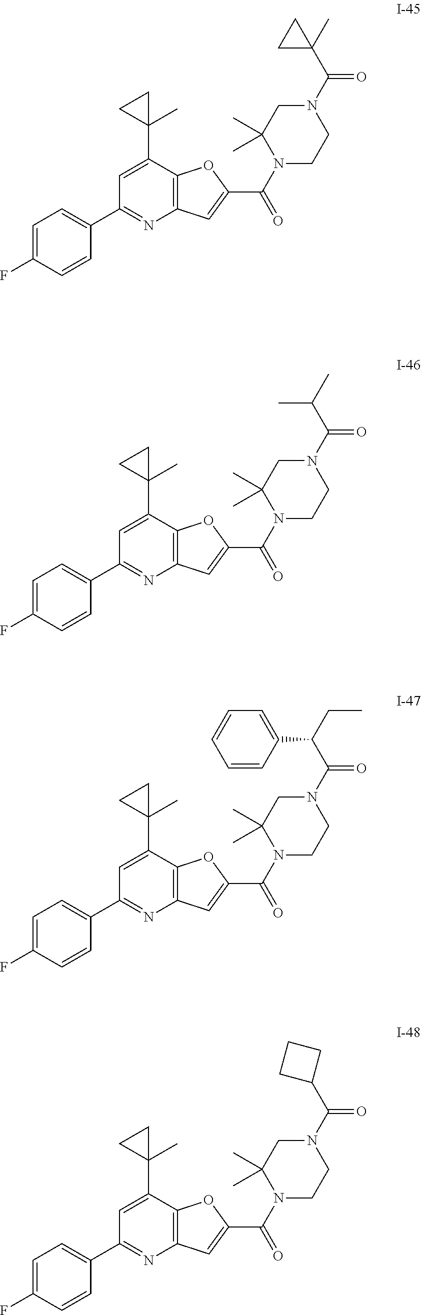 [SMILES: CC1(C(=O)N2CCN(C(=O)c3cc4nc(-c5ccc(F)cc5)cc(C5(C)CC5)c4o3)C(C)(C)C2)CC1 ; CC(C)C(=O)N1CCN(C(=O)c2cc3nc(-c4ccc(F)cc4)cc(C4(C)CC4)c3o2)C(C)(C)C1 ; CC[C@H](C(=O)N1CCN(C(=O)c2cc3nc(-c4ccc(F)cc4)cc(C4(C)CC4)c3o2)C(C)(C)C1)c1ccccc1 ; CC1(c2cc(-c3ccc(F)cc3)nc3cc(C(=O)N4CCN(C(=O)C5CCC5)CC4(C)C)oc23)CC1]

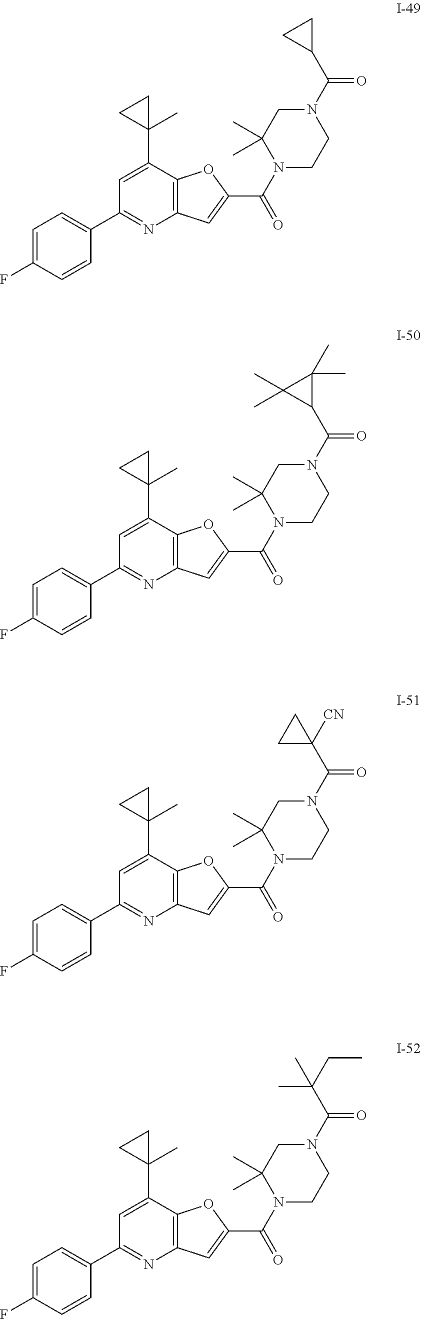 [SMILES: CC1(c2cc(-c3ccc(F)cc3)nc3cc(C(=O)N4CCN(C(=O)C5CC5)CC4(C)C)oc23)CC1 ; CC1(c2cc(-c3ccc(F)cc3)nc3cc(C(=O)N4CCN(C(=O)C5C(C)(C)C5(C)C)CC4(C)C)oc23)CC1 ; CC1(c2cc(-c3ccc(F)cc3)nc3cc(C(=O)N4CCN(C(=O)C5(C#N)CC5)CC4(C)C)oc23)CC1 ; CCC(C)(C)C(=O)N1CCN(C(=O)c2cc3nc(-c4ccc(F)cc4)cc(C4(C)CC4)c3o2)C(C)(C)C1]

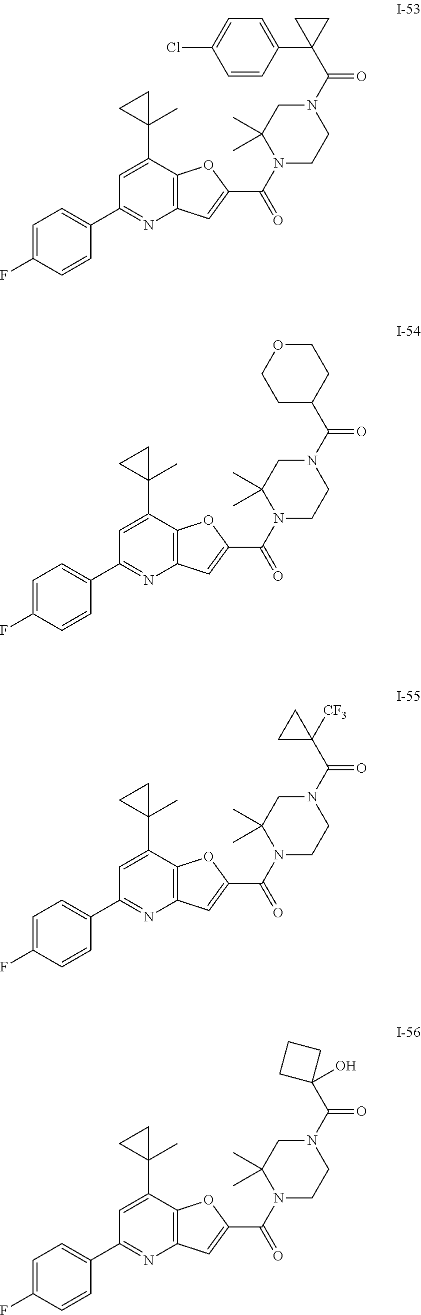 [SMILES: CC1(c2cc(-c3ccc(F)cc3)nc3cc(C(=O)N4CCN(C(=O)C5(c6ccc(Cl)cc6)CC5)CC4(C)C)oc23)CC1 ; CC1(c2cc(-c3ccc(F)cc3)nc3cc(C(=O)N4CCN(C(=O)C5CCOCC5)CC4(C)C)oc23)CC1 ; CC1(c2cc(-c3ccc(F)cc3)nc3cc(C(=O)N4CCN(C(=O)C5(C(F)(F)F)CC5)CC4(C)C)oc23)CC1 ; CC1(c2cc(-c3ccc(F)cc3)nc3cc(C(=O)N4CCN(C(=O)C5(O)CCC5)CC4(C)C)oc23)CC1]

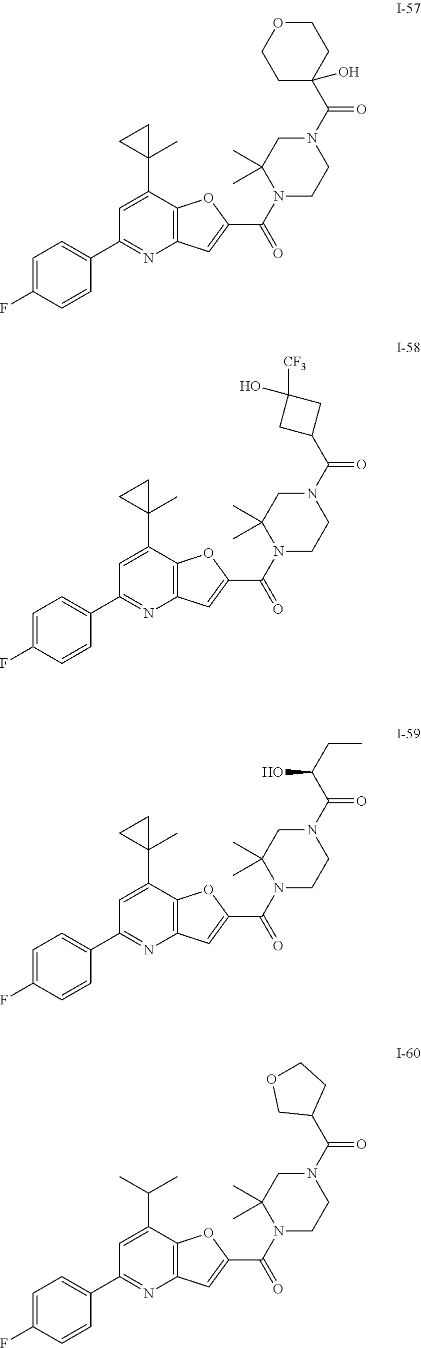 [SMILES: CC1(c2cc(-c3ccc(F)cc3)nc3cc(C(=O)N4CCN(C(=O)C5(O)CCOCC5)CC4(C)C)oc23)CC1 ; CC1(c2cc(-c3ccc(F)cc3)nc3cc(C(=O)N4CCN(C(=O)C5CC(O)(C(F)(F)F)C5)CC4(C)C)oc23)CC1 ; CC[C@H](O)C(=O)N1CCN(C(=O)c2cc3nc(-c4ccc(F)cc4)cc(C4(C)CC4)c3o2)C(C)(C)C1 ; CC(C)c1cc(-c2ccc(F)cc2)nc2cc(C(=O)N3CCN(C(=O)C4CCOC4)CC3(C)C)oc12]

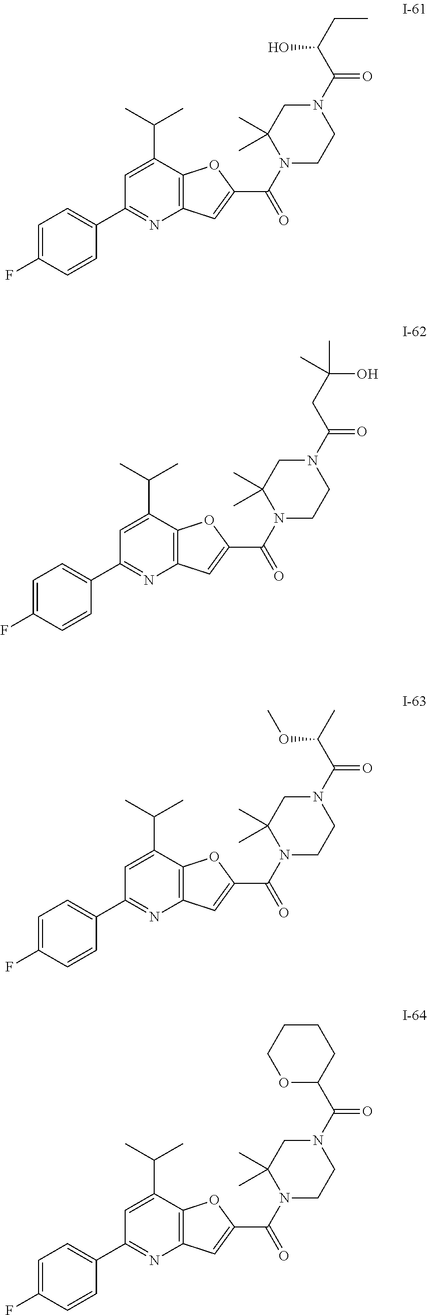 [SMILES: CC[C@@H](O)C(=O)N1CCN(C(=O)c2cc3nc(-c4ccc(F)cc4)cc(C(C)C)c3o2)C(C)(C)C1 ; CC(C)c1cc(-c2ccc(F)cc2)nc2cc(C(=O)N3CCN(C(=O)CC(C)(C)O)CC3(C)C)oc12 ; CO[C@H](C)C(=O)N1CCN(C(=O)c2cc3nc(-c4ccc(F)cc4)cc(C(C)C)c3o2)C(C)(C)C1 ; CC(C)c1cc(-c2ccc(F)cc2)nc2cc(C(=O)N3CCN(C(=O)C4CCCCO4)CC3(C)C)oc12]

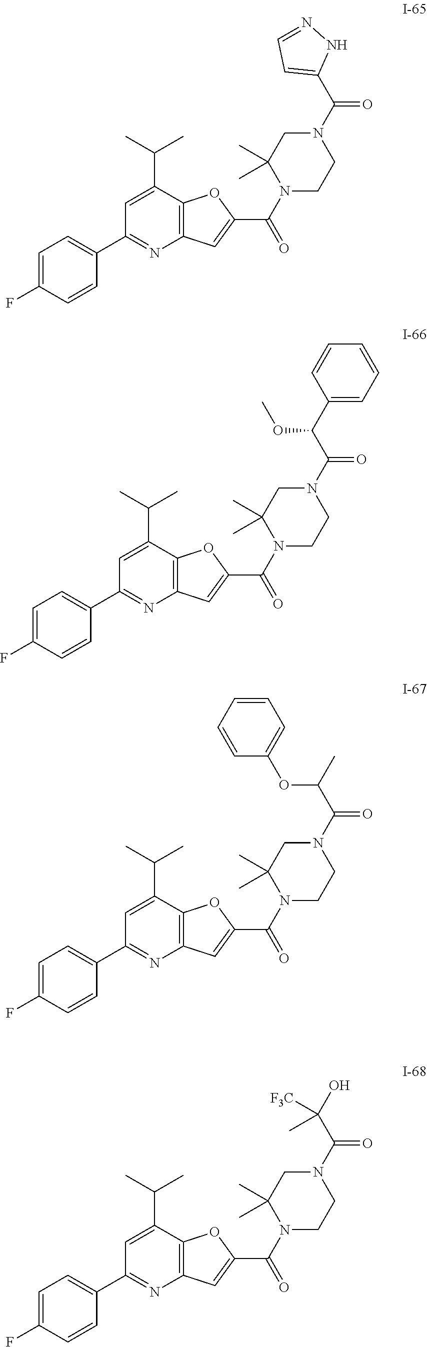 [SMILES: CC(C)c1cc(-c2ccc(F)cc2)nc2cc(C(=O)N3CCN(C(=O)c4ccn[nH]4)CC3(C)C)oc12 ; CO[C@@H](C(=O)N1CCN(C(=O)c2cc3nc(-c4ccc(F)cc4)cc(C(C)C)c3o2)C(C)(C)C1)c1ccccc1 ; CC(Oc1ccccc1)C(=O)N1CCN(C(=O)c2cc3nc(-c4ccc(F)cc4)cc(C(C)C)c3o2)C(C)(C)C1 ; CC(C)c1cc(-c2ccc(F)cc2)nc2cc(C(=O)N3CCN(C(=O)C(C)(O)C(F)(F)F)CC3(C)C)oc12]

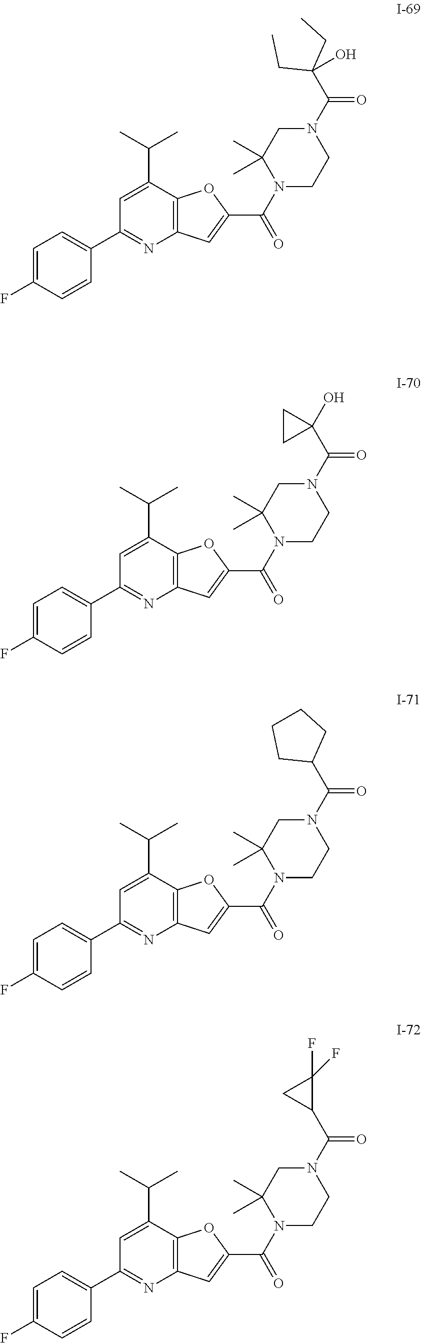 [SMILES: CCC(O)(CC)C(=O)N1CCN(C(=O)c2cc3nc(-c4ccc(F)cc4)cc(C(C)C)c3o2)C(C)(C)C1 ; CC(C)c1cc(-c2ccc(F)cc2)nc2cc(C(=O)N3CCN(C(=O)C4(O)CC4)CC3(C)C)oc12 ; CC(C)c1cc(-c2ccc(F)cc2)nc2cc(C(=O)N3CCN(C(=O)C4CCCC4)CC3(C)C)oc12 ; CC(C)c1cc(-c2ccc(F)cc2)nc2cc(C(=O)N3CCN(C(=O)C4CC4(F)F)CC3(C)C)oc12]

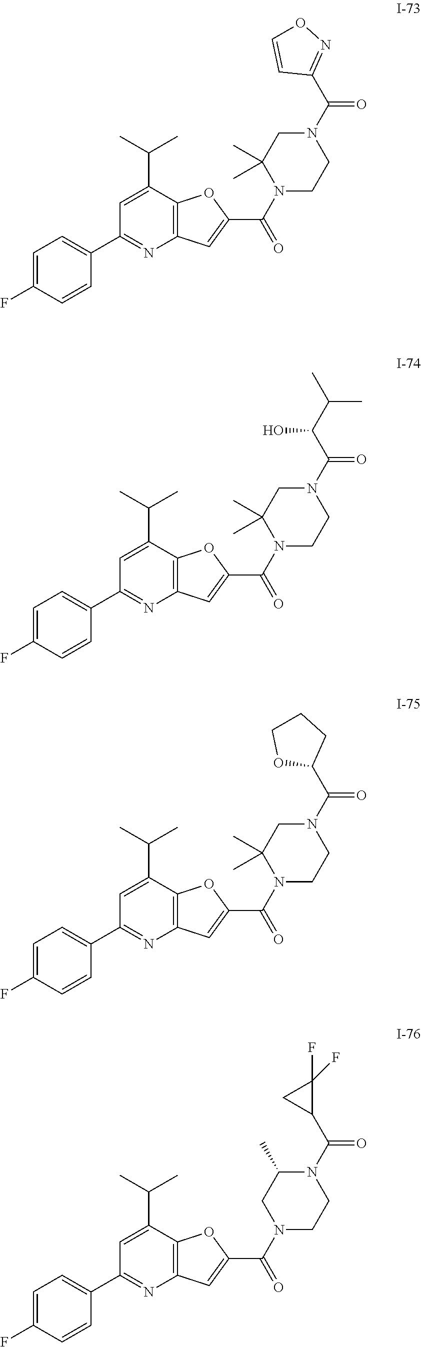 [SMILES: CC(C)c1cc(-c2ccc(F)cc2)nc2cc(C(=O)N3CCN(C(=O)c4ccon4)CC3(C)C)oc12 ; CC(C)c1cc(-c2ccc(F)cc2)nc2cc(C(=O)N3CCN(C(=O)[C@H](O)C(C)C)CC3(C)C)oc12 ; CC(C)c1cc(-c2ccc(F)cc2)nc2cc(C(=O)N3CCN(C(=O)[C@H]4CCCO4)CC3(C)C)oc12 ; CC(C)c1cc(-c2ccc(F)cc2)nc2cc(C(=O)N3CCN(C(=O)C4CC4(F)F)[C@@H](C)C3)oc12]

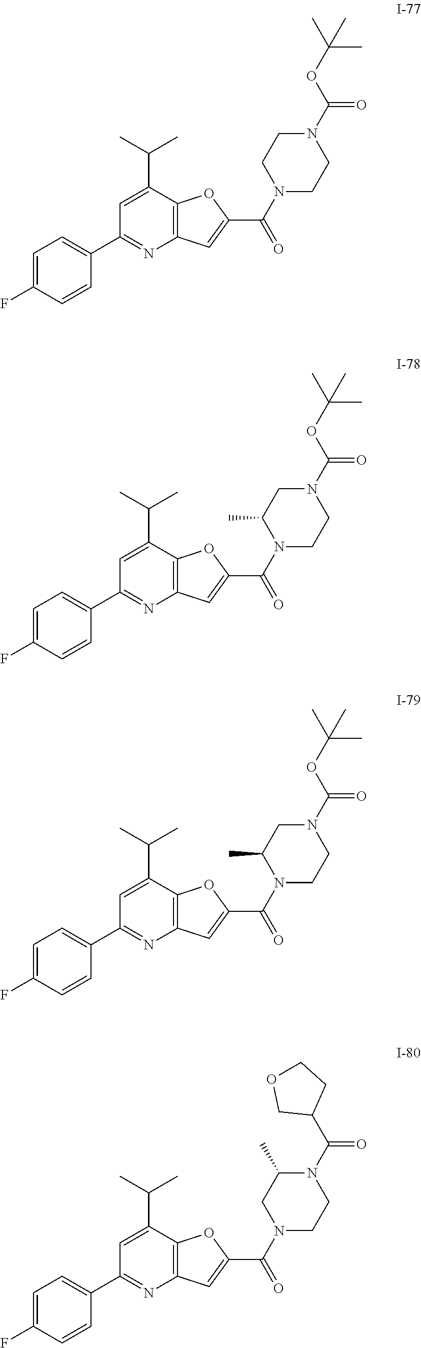 [SMILES: CC(C)c1cc(-c2ccc(F)cc2)nc2cc(C(=O)N3CCN(C(=O)OC(C)(C)C)CC3)oc12 ; CC(C)c1cc(-c2ccc(F)cc2)nc2cc(C(=O)N3CCN(C(=O)OC(C)(C)C)C[C@H]3C)oc12 ; CC(C)c1cc(-c2ccc(F)cc2)nc2cc(C(=O)N3CCN(C(=O)OC(C)(C)C)C[C@@H]3C)oc12 ; CC(C)c1cc(-c2ccc(F)cc2)nc2cc(C(=O)N3CCN(C(=O)C4CCOC4)[C@@H](C)C3)oc12]

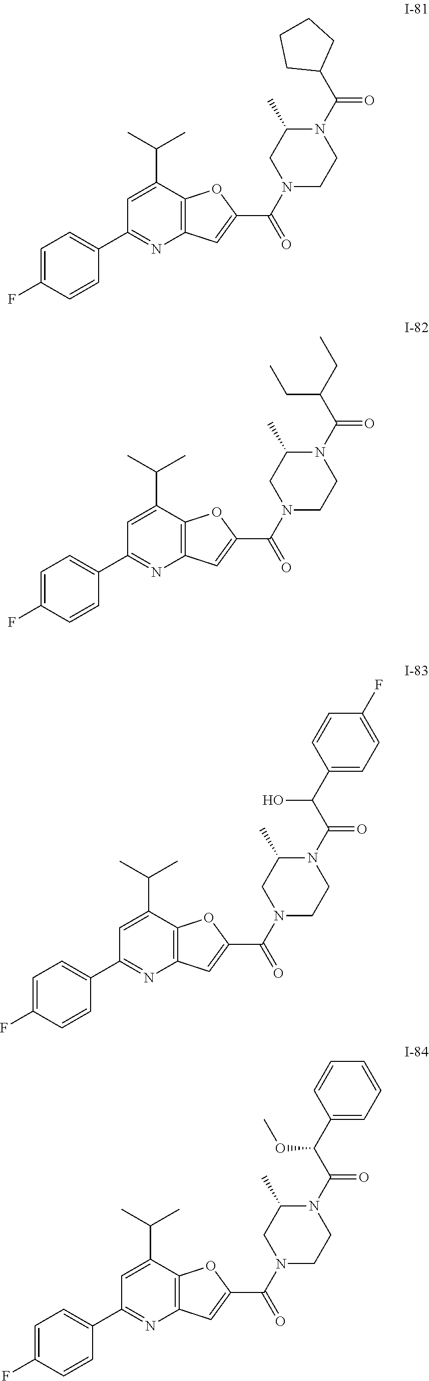 [SMILES: CC(C)c1cc(-c2ccc(F)cc2)nc2cc(C(=O)N3CCN(C(=O)C4CCCC4)[C@@H](C)C3)oc12 ; CCC(CC)C(=O)N1CCN(C(=O)c2cc3nc(-c4ccc(F)cc4)cc(C(C)C)c3o2)C[C@@H]1C ; CC(C)c1cc(-c2ccc(F)cc2)nc2cc(C(=O)N3CCN(C(=O)C(O)c4ccc(F)cc4)[C@@H](C)C3)oc12 ; CO[C@@H](C(=O)N1CCN(C(=O)c2cc3nc(-c4ccc(F)cc4)cc(C(C)C)c3o2)C[C@@H]1C)c1ccccc1]

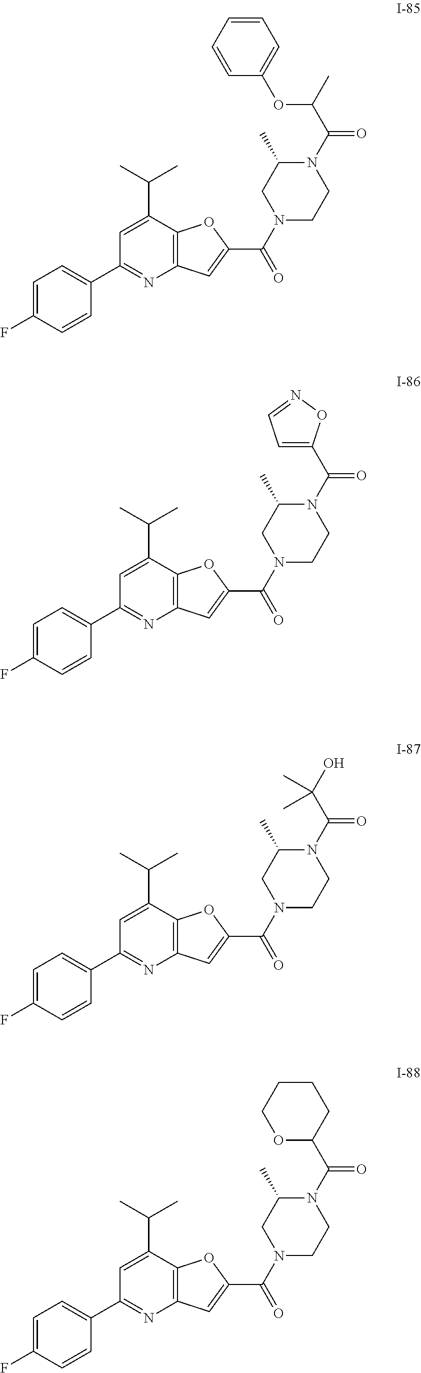 [SMILES: CC(Oc1ccccc1)C(=O)N1CCN(C(=O)c2cc3nc(-c4ccc(F)cc4)cc(C(C)C)c3o2)C[C@@H]1C ; CC(C)c1cc(-c2ccc(F)cc2)nc2cc(C(=O)N3CCN(C(=O)c4ccno4)[C@@H](C)C3)oc12 ; CC(C)c1cc(-c2ccc(F)cc2)nc2cc(C(=O)N3CCN(C(=O)C(C)(C)O)[C@@H](C)C3)oc12 ; CC(C)c1cc(-c2ccc(F)cc2)nc2cc(C(=O)N3CCN(C(=O)C4CCCCO4)[C@@H](C)C3)oc12]

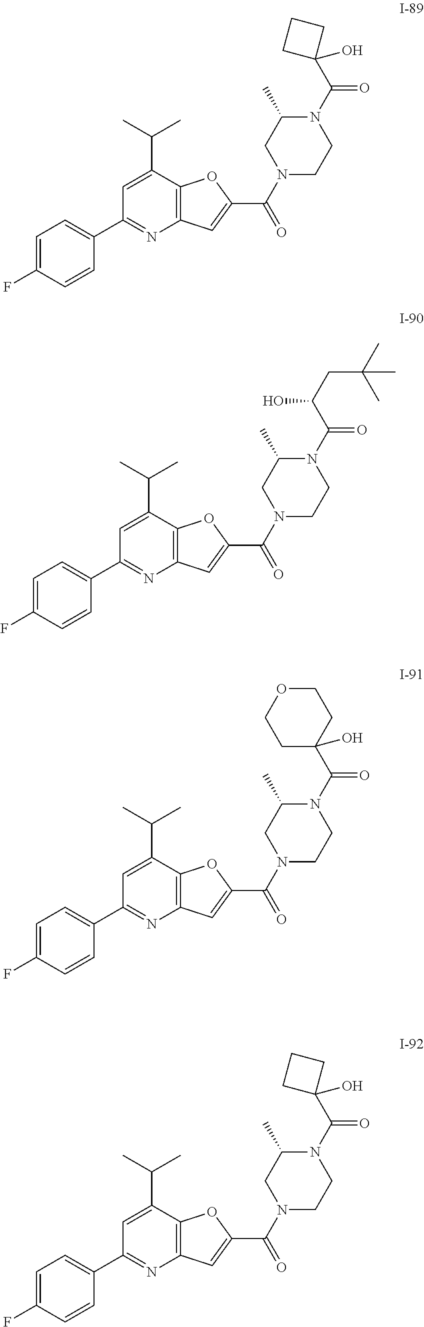 [SMILES: CC(C)c1cc(-c2ccc(F)cc2)nc2cc(C(=O)N3CCN(C(=O)C4(O)CCC4)[C@@H](C)C3)oc12 ; CC(C)c1cc(-c2ccc(F)cc2)nc2cc(C(=O)N3CCN(C(=O)[C@H](O)CC(C)(C)C)[C@@H](C)C3)oc12 ; CC(C)c1cc(-c2ccc(F)cc2)nc2cc(C(=O)N3CCN(C(=O)C4(O)CCOCC4)[C@@H](C)C3)oc12 ; CC(C)c1cc(-c2ccc(F)cc2)nc2cc(C(=O)N3CCN(C(=O)C4(O)CCC4)[C@@H](C)C3)oc12]

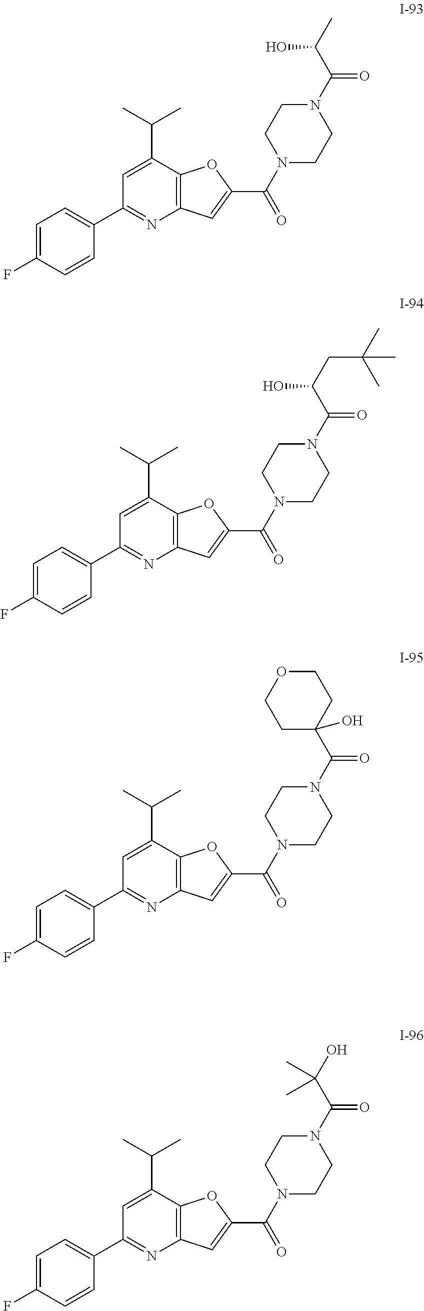 [SMILES: CC(C)c1cc(-c2ccc(F)cc2)nc2cc(C(=O)N3CCN(C(=O)[C@@H](C)O)CC3)oc12 ; CC(C)c1cc(-c2ccc(F)cc2)nc2cc(C(=O)N3CCN(C(=O)[C@H](O)CC(C)(C)C)CC3)oc12 ; CC(C)c1cc(-c2ccc(F)cc2)nc2cc(C(=O)N3CCN(C(=O)C4(O)CCOCC4)CC3)oc12 ; CC(C)c1cc(-c2ccc(F)cc2)nc2cc(C(=O)N3CCN(C(=O)C(C)(C)O)CC3)oc12]

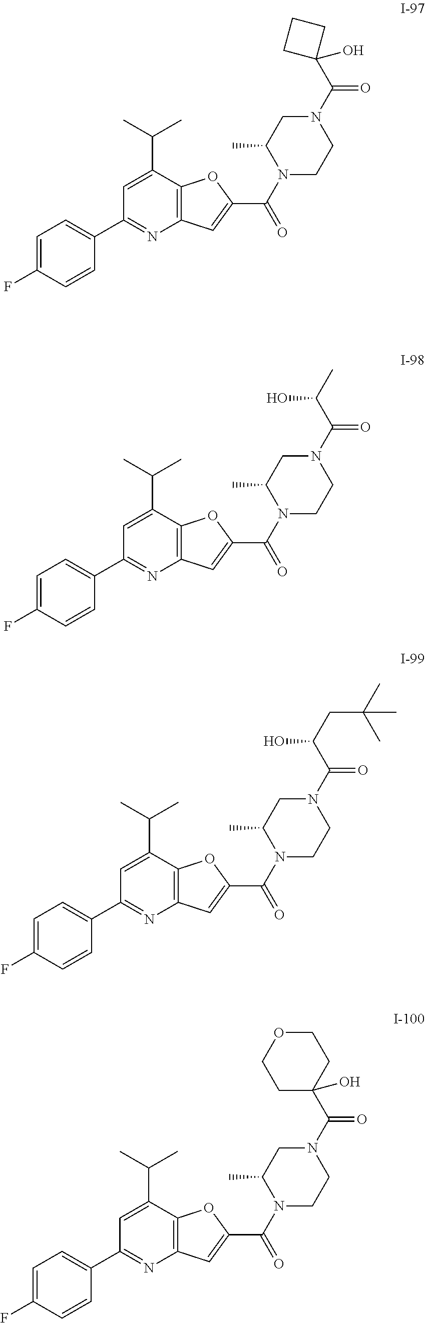 [SMILES: CC(C)c1cc(-c2ccc(F)cc2)nc2cc(C(=O)N3CCN(C(=O)C4(O)CCC4)C[C@H]3C)oc12 ; CC(C)c1cc(-c2ccc(F)cc2)nc2cc(C(=O)N3CCN(C(=O)[C@@H](C)O)C[C@H]3C)oc12 ; CC(C)c1cc(-c2ccc(F)cc2)nc2cc(C(=O)N3CCN(C(=O)[C@H](O)CC(C)(C)C)C[C@H]3C)oc12 ; CC(C)c1cc(-c2ccc(F)cc2)nc2cc(C(=O)N3CCN(C(=O)C4(O)CCOCC4)C[C@H]3C)oc12]

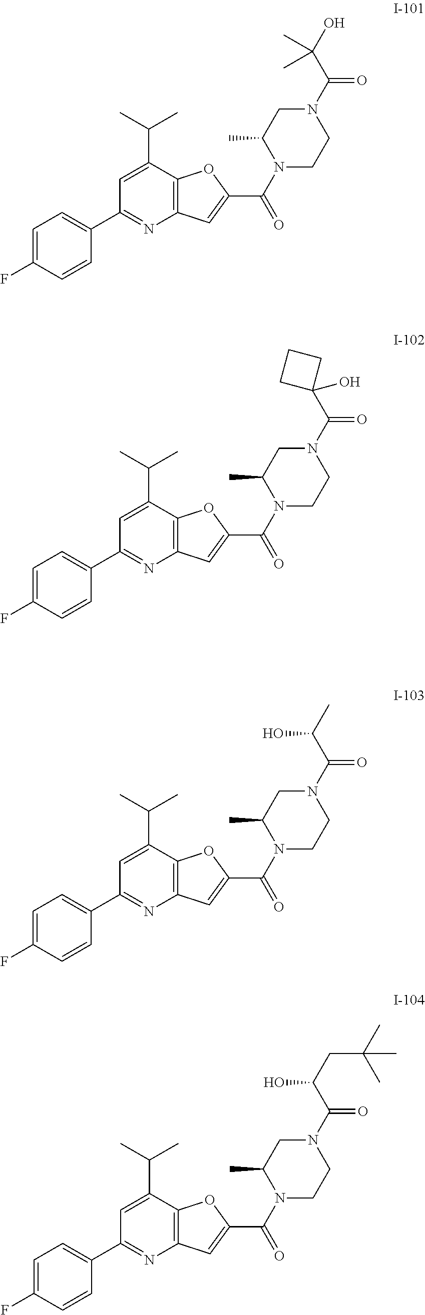 [SMILES: CC(C)c1cc(-c2ccc(F)cc2)nc2cc(C(=O)N3CCN(C(=O)C(C)(C)O)C[C@H]3C)oc12 ; CC(C)c1cc(-c2ccc(F)cc2)nc2cc(C(=O)N3CCN(C(=O)C4(O)CCC4)C[C@@H]3C)oc12 ; CC(C)c1cc(-c2ccc(F)cc2)nc2cc(C(=O)N3CCN(C(=O)[C@@H](C)O)C[C@@H]3C)oc12 ; CC(C)c1cc(-c2ccc(F)cc2)nc2cc(C(=O)N3CCN(C(=O)[C@H](O)CC(C)(C)C)C[C@@H]3C)oc12]

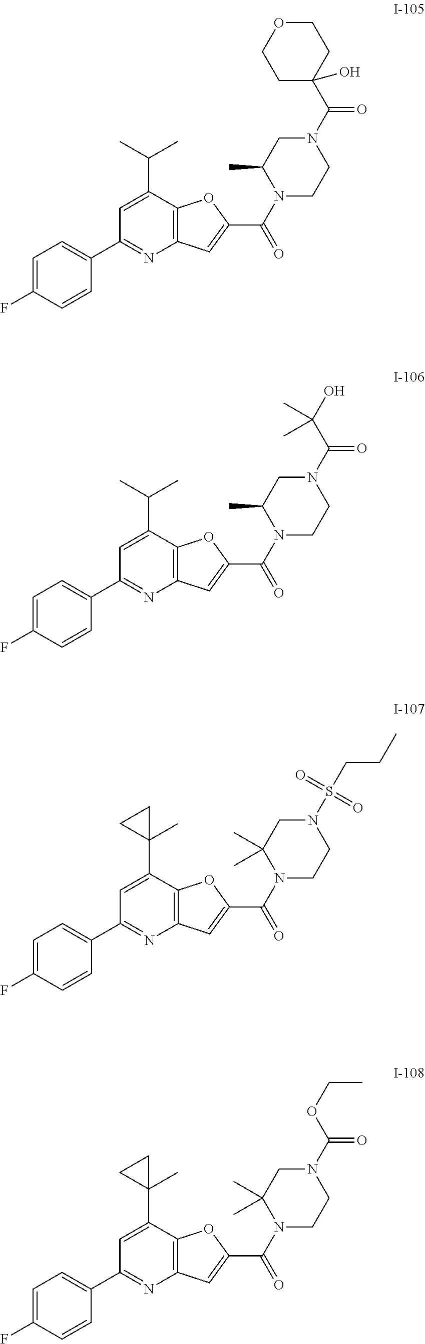 [SMILES: CC(C)c1cc(-c2ccc(F)cc2)nc2cc(C(=O)N3CCN(C(=O)C4(O)CCOCC4)C[C@@H]3C)oc12 ; CC(C)c1cc(-c2ccc(F)cc2)nc2cc(C(=O)N3CCN(C(=O)C(C)(C)O)C[C@@H]3C)oc12 ; CCCS(=O)(=O)N1CCN(C(=O)c2cc3nc(-c4ccc(F)cc4)cc(C4(C)CC4)c3o2)C(C)(C)C1 ; CCOC(=O)N1CCN(C(=O)c2cc3nc(-c4ccc(F)cc4)cc(C4(C)CC4)c3o2)C(C)(C)C1]

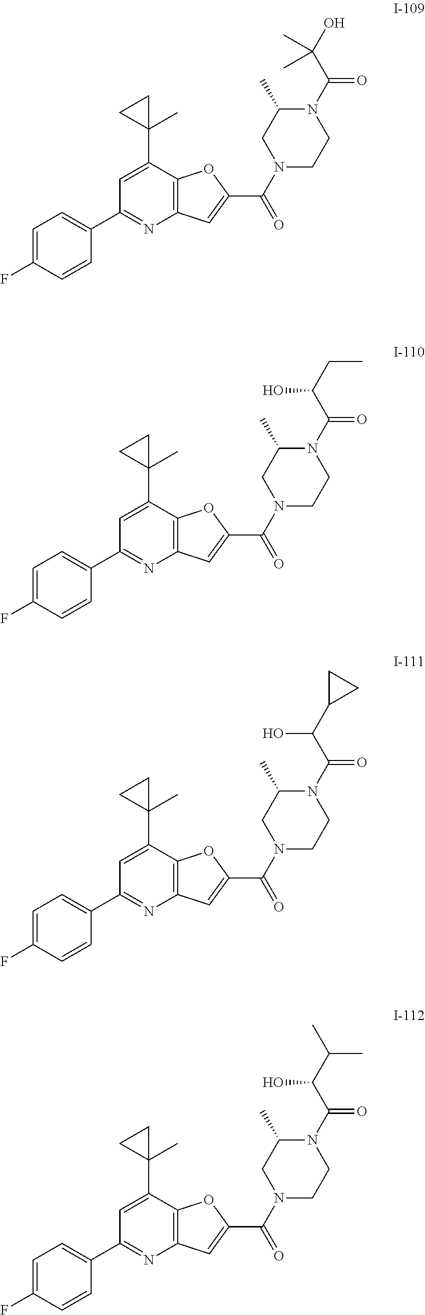 [SMILES: C[C@H]1CN(C(=O)c2cc3nc(-c4ccc(F)cc4)cc(C4(C)CC4)c3o2)CCN1C(=O)C(C)(C)O ; CC[C@@H](O)C(=O)N1CCN(C(=O)c2cc3nc(-c4ccc(F)cc4)cc(C4(C)CC4)c3o2)C[C@@H]1C ; C[C@H]1CN(C(=O)c2cc3nc(-c4ccc(F)cc4)cc(C4(C)CC4)c3o2)CCN1C(=O)C(O)C1CC1 ; CC(C)[C@@H](O)C(=O)N1CCN(C(=O)c2cc3nc(-c4ccc(F)cc4)cc(C4(C)CC4)c3o2)C[C@@H]1C]

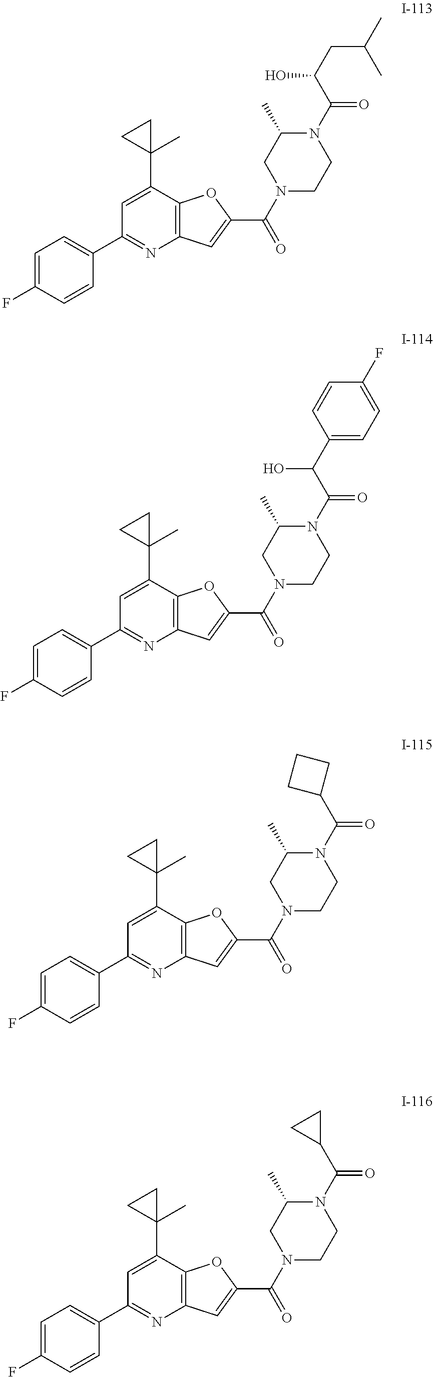 [SMILES: CC(C)C[C@@H](O)C(=O)N1CCN(C(=O)c2cc3nc(-c4ccc(F)cc4)cc(C4(C)CC4)c3o2)C[C@@H]1C ; C[C@H]1CN(C(=O)c2cc3nc(-c4ccc(F)cc4)cc(C4(C)CC4)c3o2)CCN1C(=O)C(O)c1ccc(F)cc1 ; C[C@H]1CN(C(=O)c2cc3nc(-c4ccc(F)cc4)cc(C4(C)CC4)c3o2)CCN1C(=O)C1CCC1 ; C[C@H]1CN(C(=O)c2cc3nc(-c4ccc(F)cc4)cc(C4(C)CC4)c3o2)CCN1C(=O)C1CC1]

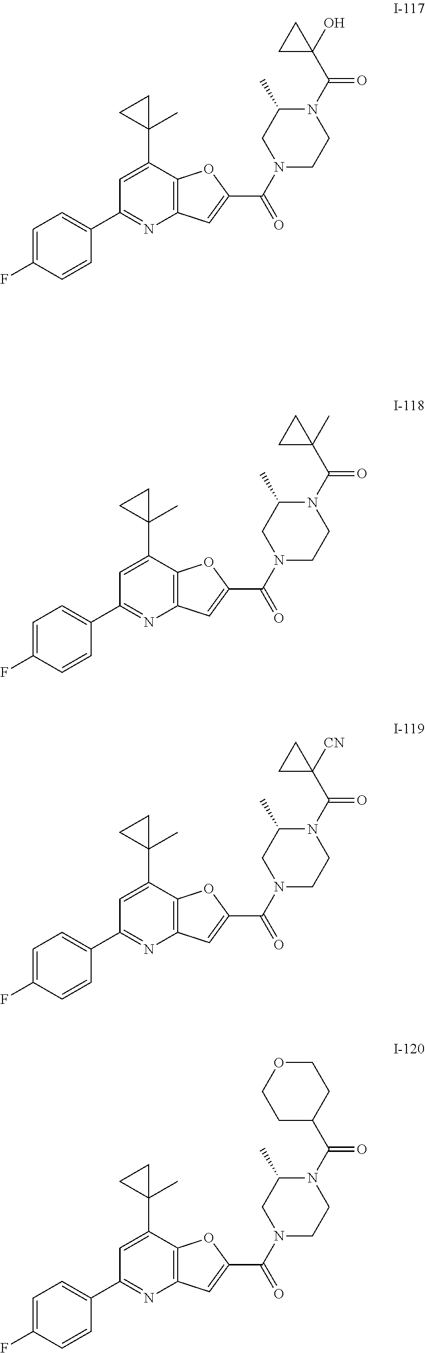 [SMILES: C[C@H]1CN(C(=O)c2cc3nc(-c4ccc(F)cc4)cc(C4(C)CC4)c3o2)CCN1C(=O)C1(O)CC1 ; C[C@H]1CN(C(=O)c2cc3nc(-c4ccc(F)cc4)cc(C4(C)CC4)c3o2)CCN1C(=O)C1(C)CC1 ; C[C@H]1CN(C(=O)c2cc3nc(-c4ccc(F)cc4)cc(C4(C)CC4)c3o2)CCN1C(=O)C1(C#N)CC1 ; C[C@H]1CN(C(=O)c2cc3nc(-c4ccc(F)cc4)cc(C4(C)CC4)c3o2)CCN1C(=O)C1CCOCC1]

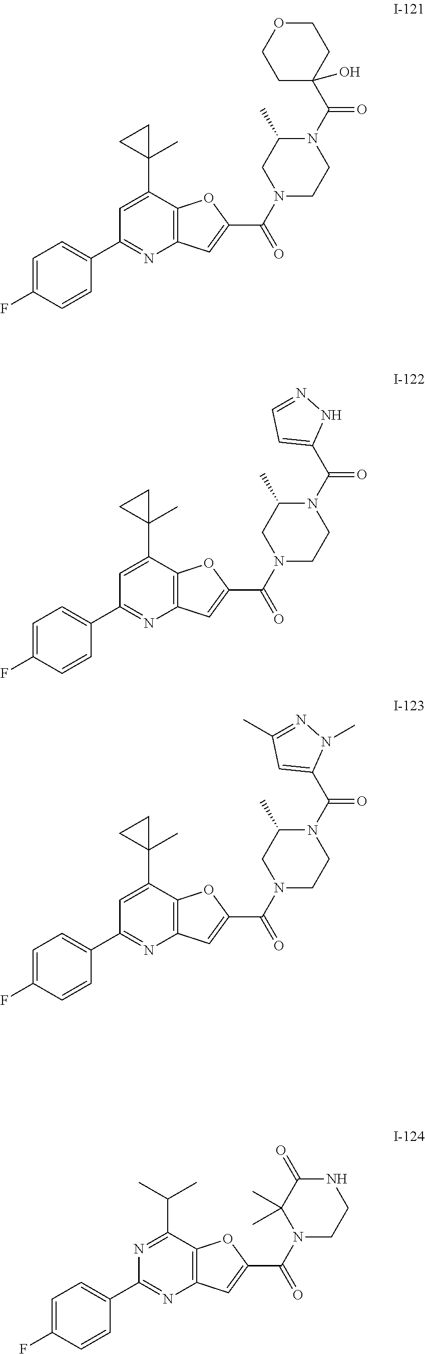 [SMILES: C[C@H]1CN(C(=O)c2cc3nc(-c4ccc(F)cc4)cc(C4(C)CC4)c3o2)CCN1C(=O)C1(O)CCOCC1 ; C[C@H]1CN(C(=O)c2cc3nc(-c4ccc(F)cc4)cc(C4(C)CC4)c3o2)CCN1C(=O)c1ccn[nH]1 ; Cc1cc(C(=O)N2CCN(C(=O)c3cc4nc(-c5ccc(F)cc5)cc(C5(C)CC5)c4o3)C[C@@H]2C)n(C)n1 ; CC(C)c1nc(-c2ccc(F)cc2)nc2cc(C(=O)N3CCNC(=O)C3(C)C)oc12]

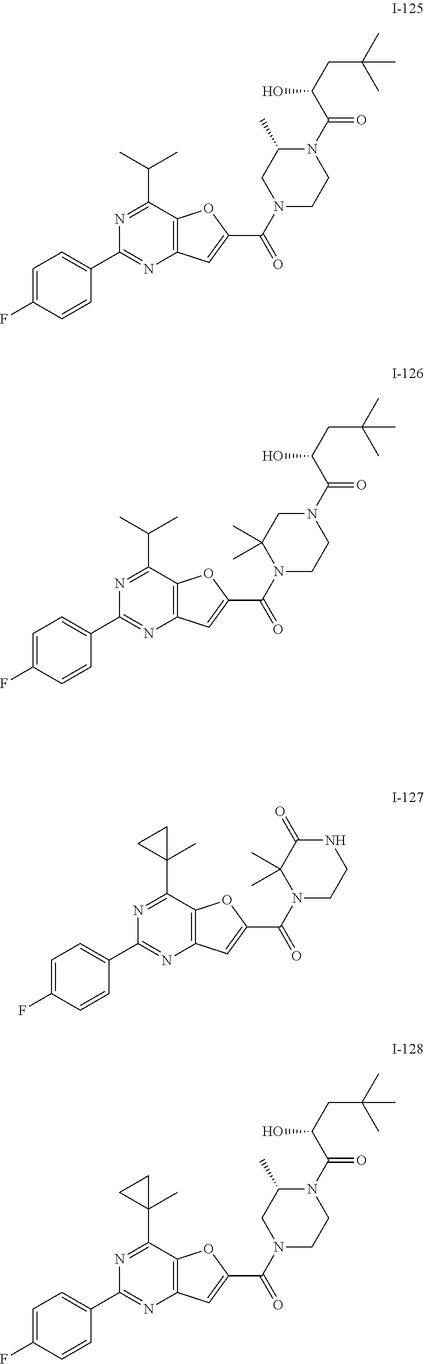 [SMILES: CC(C)c1nc(-c2ccc(F)cc2)nc2cc(C(=O)N3CCN(C(=O)[C@H](O)CC(C)(C)C)[C@@H](C)C3)oc12 ; CC(C)c1nc(-c2ccc(F)cc2)nc2cc(C(=O)N3CCN(C(=O)[C@H](O)CC(C)(C)C)CC3(C)C)oc12 ; CC1(c2nc(-c3ccc(F)cc3)nc3cc(C(=O)N4CCNC(=O)C4(C)C)oc23)CC1 ; C[C@H]1CN(C(=O)c2cc3nc(-c4ccc(F)cc4)nc(C4(C)CC4)c3o2)CCN1C(=O)[C@H](O)CC(C)(C)C]

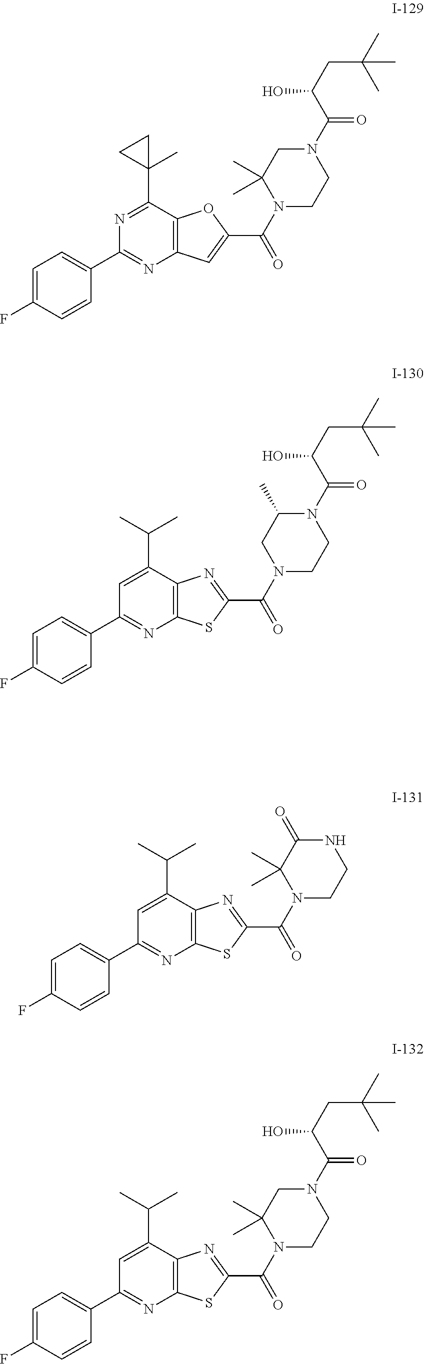 [SMILES: CC(C)(C)C[C@@H](O)C(=O)N1CCN(C(=O)c2cc3nc(-c4ccc(F)cc4)nc(C4(C)CC4)c3o2)C(C)(C)C1 ; CC(C)c1cc(-c2ccc(F)cc2)nc2sc(C(=O)N3CCN(C(=O)[C@H](O)CC(C)(C)C)[C@@H](C)C3)nc12 ; CC(C)c1cc(-c2ccc(F)cc2)nc2sc(C(=O)N3CCNC(=O)C3(C)C)nc12 ; CC(C)c1cc(-c2ccc(F)cc2)nc2sc(C(=O)N3CCN(C(=O)[C@H](O)CC(C)(C)C)CC3(C)C)nc12]

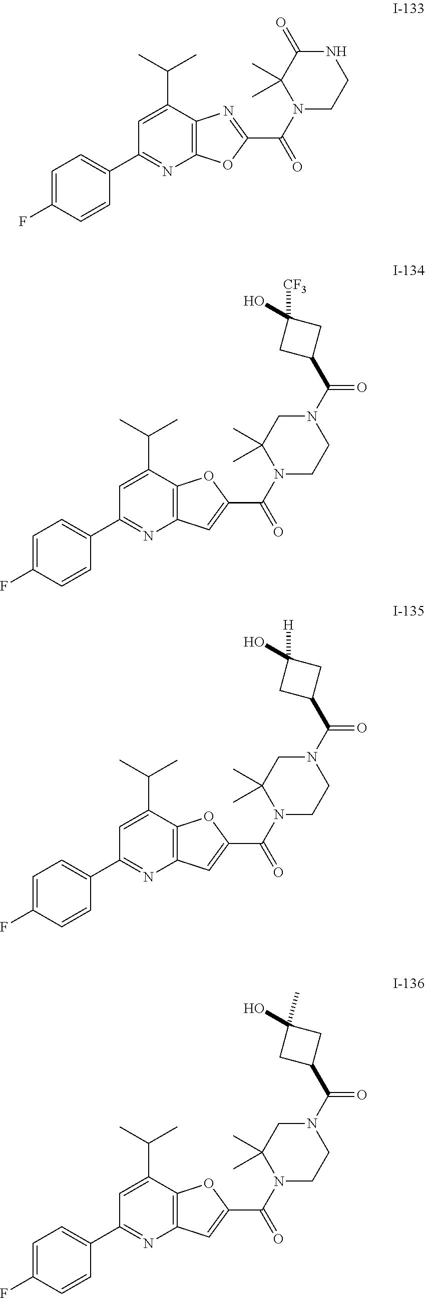 [SMILES: CC(C)c1cc(-c2ccc(F)cc2)nc2oc(C(=O)N3CCNC(=O)C3(C)C)nc12 ; CC(C)c1cc(-c2ccc(F)cc2)nc2cc(C(=O)N3CCN(C(=O)[C@H]4C[C@](O)(C(F)(F)F)C4)CC3(C)C)oc12 ; CC(C)c1cc(-c2ccc(F)cc2)nc2cc(C(=O)N3CCN(C(=O)[C@H]4C[C@@H](O)C4)CC3(C)C)oc12 ; CC(C)c1cc(-c2ccc(F)cc2)nc2cc(C(=O)N3CCN(C(=O)[C@H]4C[C@@](C)(O)C4)CC3(C)C)oc12]

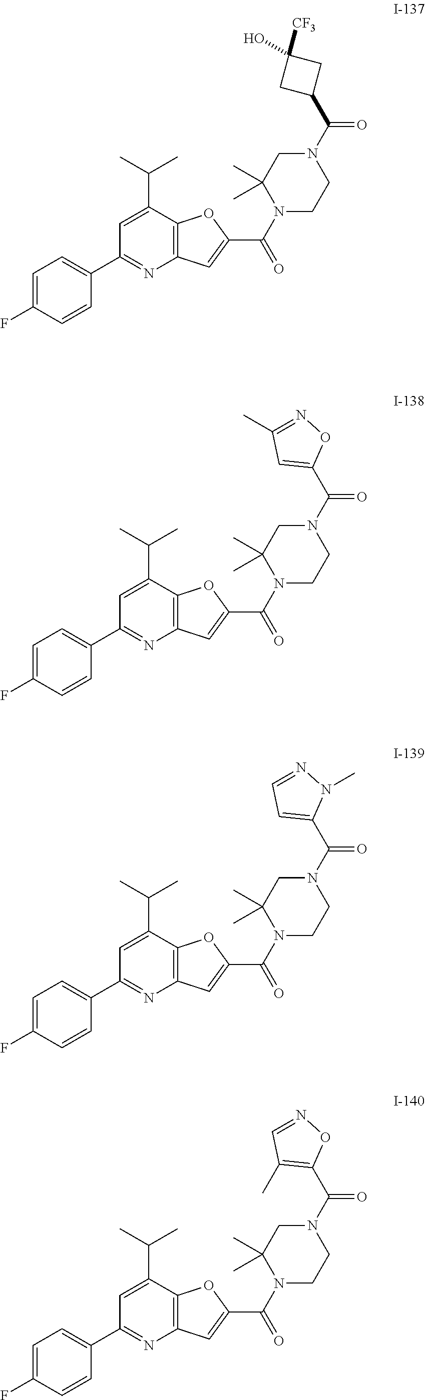 [SMILES: CC(C)c1cc(-c2ccc(F)cc2)nc2cc(C(=O)N3CCN(C(=O)[C@H]4C[C@@](O)(C(F)(F)F)C4)CC3(C)C)oc12 ; Cc1cc(C(=O)N2CCN(C(=O)c3cc4nc(-c5ccc(F)cc5)cc(C(C)C)c4o3)C(C)(C)C2)on1 ; CC(C)c1cc(-c2ccc(F)cc2)nc2cc(C(=O)N3CCN(C(=O)c4ccnn4C)CC3(C)C)oc12 ; Cc1cnoc1C(=O)N1CCN(C(=O)c2cc3nc(-c4ccc(F)cc4)cc(C(C)C)c3o2)C(C)(C)C1]

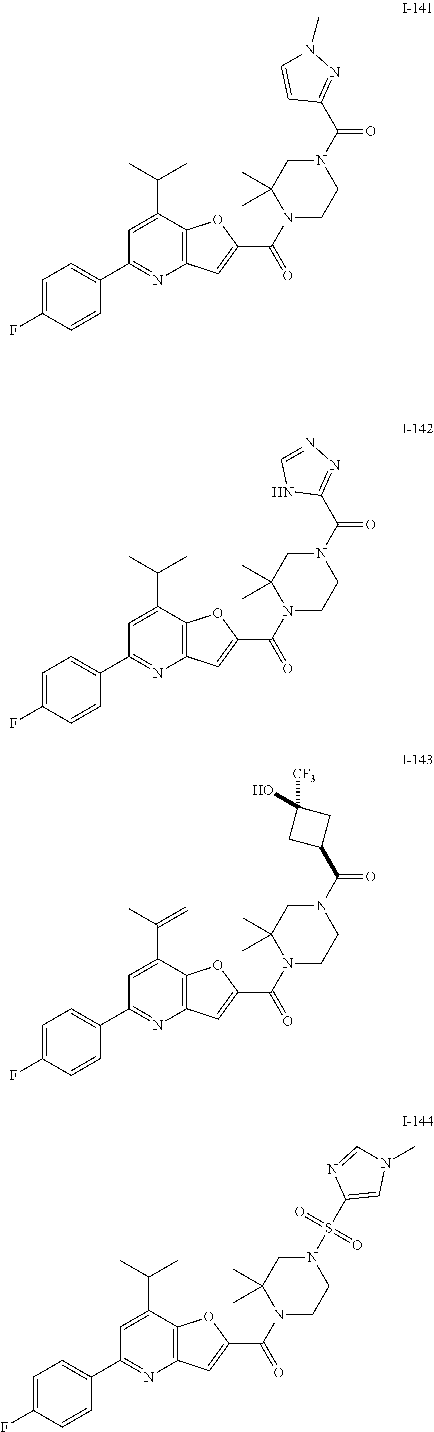 [SMILES: CC(C)c1cc(-c2ccc(F)cc2)nc2cc(C(=O)N3CCN(C(=O)c4ccn(C)n4)CC3(C)C)oc12 ; CC(C)c1cc(-c2ccc(F)cc2)nc2cc(C(=O)N3CCN(C(=O)c4nnc[nH]4)CC3(C)C)oc12 ; C=C(C)c1cc(-c2ccc(F)cc2)nc2cc(C(=O)N3CCN(C(=O)[C@H]4C[C@](O)(C(F)(F)F)C4)CC3(C)C)oc12 ; CC(C)c1cc(-c2ccc(F)cc2)nc2cc(C(=O)N3CCN(S(=O)(=O)c4cn(C)cn4)CC3(C)C)oc12]

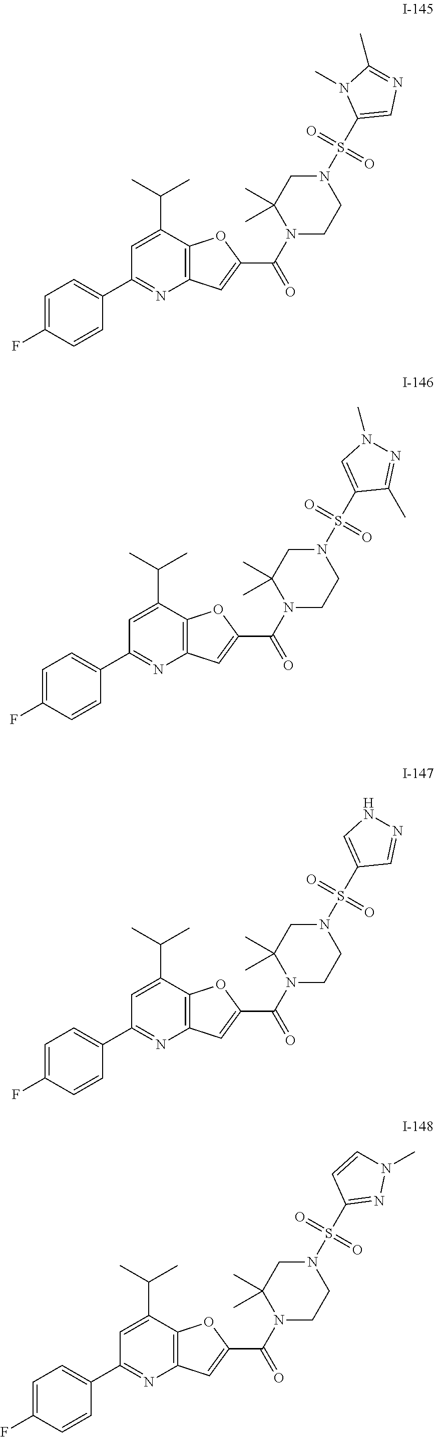 [SMILES: Cc1ncc(S(=O)(=O)N2CCN(C(=O)c3cc4nc(-c5ccc(F)cc5)cc(C(C)C)c4o3)C(C)(C)C2)n1C ; Cc1nn(C)cc1S(=O)(=O)N1CCN(C(=O)c2cc3nc(-c4ccc(F)cc4)cc(C(C)C)c3o2)C(C)(C)C1 ; CC(C)c1cc(-c2ccc(F)cc2)nc2cc(C(=O)N3CCN(S(=O)(=O)c4cn[nH]c4)CC3(C)C)oc12 ; CC(C)c1cc(-c2ccc(F)cc2)nc2cc(C(=O)N3CCN(S(=O)(=O)c4ccn(C)n4)CC3(C)C)oc12]

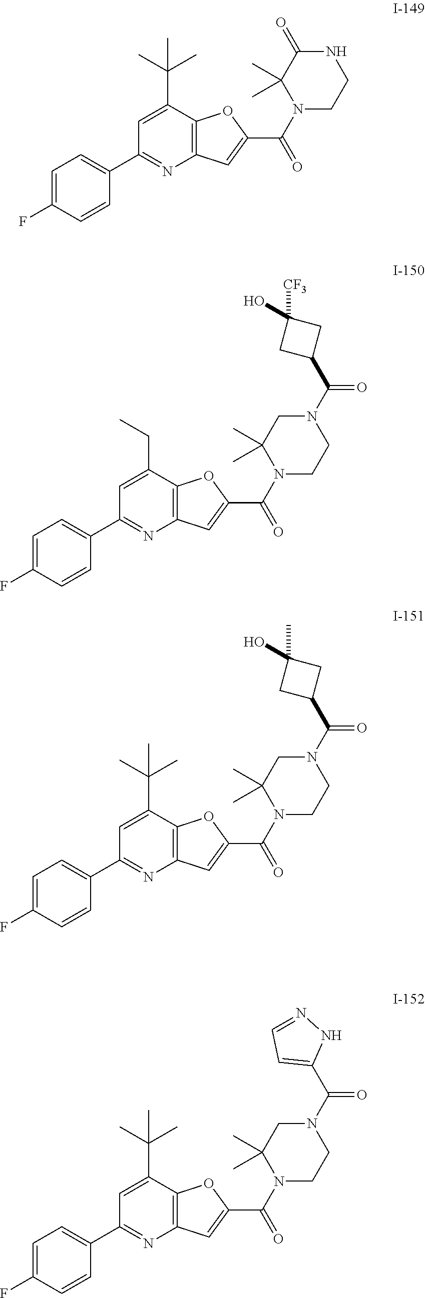 [SMILES: CC(C)(C)c1cc(-c2ccc(F)cc2)nc2cc(C(=O)N3CCNC(=O)C3(C)C)oc12 ; CCc1cc(-c2ccc(F)cc2)nc2cc(C(=O)N3CCN(C(=O)[C@H]4C[C@](O)(C(F)(F)F)C4)CC3(C)C)oc12 ; CC(C)(C)c1cc(-c2ccc(F)cc2)nc2cc(C(=O)N3CCN(C(=O)[C@H]4C[C@@](C)(O)C4)CC3(C)C)oc12 ; CC(C)(C)c1cc(-c2ccc(F)cc2)nc2cc(C(=O)N3CCN(C(=O)c4ccn[nH]4)CC3(C)C)oc12]

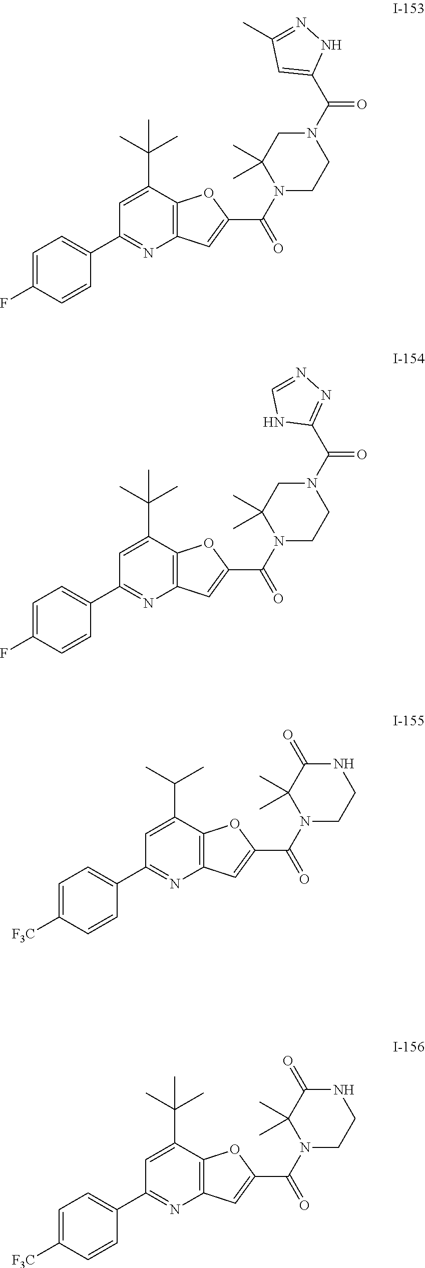 [SMILES: Cc1cc(C(=O)N2CCN(C(=O)c3cc4nc(-c5ccc(F)cc5)cc(C(C)(C)C)c4o3)C(C)(C)C2)[nH]n1 ; CC(C)(C)c1cc(-c2ccc(F)cc2)nc2cc(C(=O)N3CCN(C(=O)c4nnc[nH]4)CC3(C)C)oc12 ; CC(C)c1cc(-c2ccc(C(F)(F)F)cc2)nc2cc(C(=O)N3CCNC(=O)C3(C)C)oc12 ; CC(C)(C)c1cc(-c2ccc(C(F)(F)F)cc2)nc2cc(C(=O)N3CCNC(=O)C3(C)C)oc12]

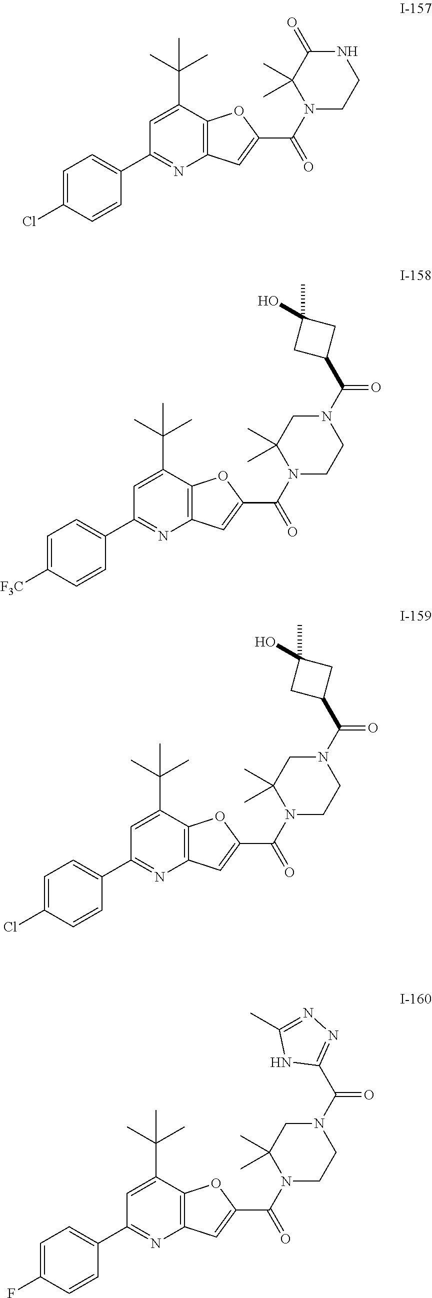 [SMILES: CC(C)(C)c1cc(-c2ccc(Cl)cc2)nc2cc(C(=O)N3CCNC(=O)C3(C)C)oc12 ; CC(C)(C)c1cc(-c2ccc(C(F)(F)F)cc2)nc2cc(C(=O)N3CCN(C(=O)[C@H]4C[C@@](C)(O)C4)CC3(C)C)oc12 ; CC(C)(C)c1cc(-c2ccc(Cl)cc2)nc2cc(C(=O)N3CCN(C(=O)[C@H]4C[C@@](C)(O)C4)CC3(C)C)oc12 ; Cc1nnc(C(=O)N2CCN(C(=O)c3cc4nc(-c5ccc(F)cc5)cc(C(C)(C)C)c4o3)C(C)(C)C2)[nH]1]

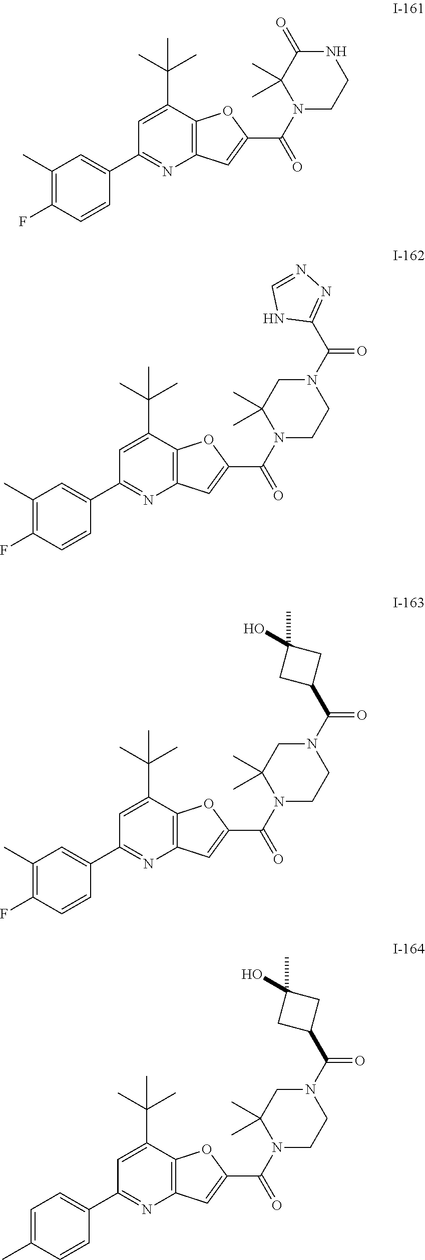 [SMILES: Cc1cc(-c2cc(C(C)(C)C)c3oc(C(=O)N4CCNC(=O)C4(C)C)cc3n2)ccc1F ; Cc1cc(-c2cc(C(C)(C)C)c3oc(C(=O)N4CCN(C(=O)c5nnc[nH]5)CC4(C)C)cc3n2)ccc1F ; Cc1cc(-c2cc(C(C)(C)C)c3oc(C(=O)N4CCN(C(=O)[C@H]5C[C@@](C)(O)C5)CC4(C)C)cc3n2)ccc1F ; Cc1ccc(-c2cc(C(C)(C)C)c3oc(C(=O)N4CCN(C(=O)[C@H]5C[C@@](C)(O)C5)CC4(C)C)cc3n2)cc1]

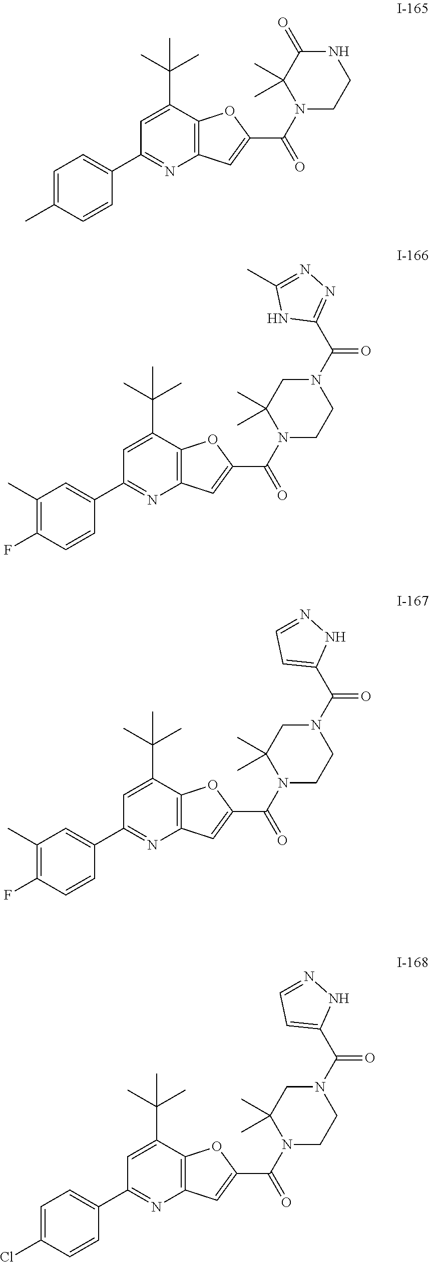 [SMILES: Cc1ccc(-c2cc(C(C)(C)C)c3oc(C(=O)N4CCNC(=O)C4(C)C)cc3n2)cc1 ; Cc1nnc(C(=O)N2CCN(C(=O)c3cc4nc(-c5ccc(F)c(C)c5)cc(C(C)(C)C)c4o3)C(C)(C)C2)[nH]1 ; Cc1cc(-c2cc(C(C)(C)C)c3oc(C(=O)N4CCN(C(=O)c5ccn[nH]5)CC4(C)C)cc3n2)ccc1F ; CC(C)(C)c1cc(-c2ccc(Cl)cc2)nc2cc(C(=O)N3CCN(C(=O)c4ccn[nH]4)CC3(C)C)oc12]

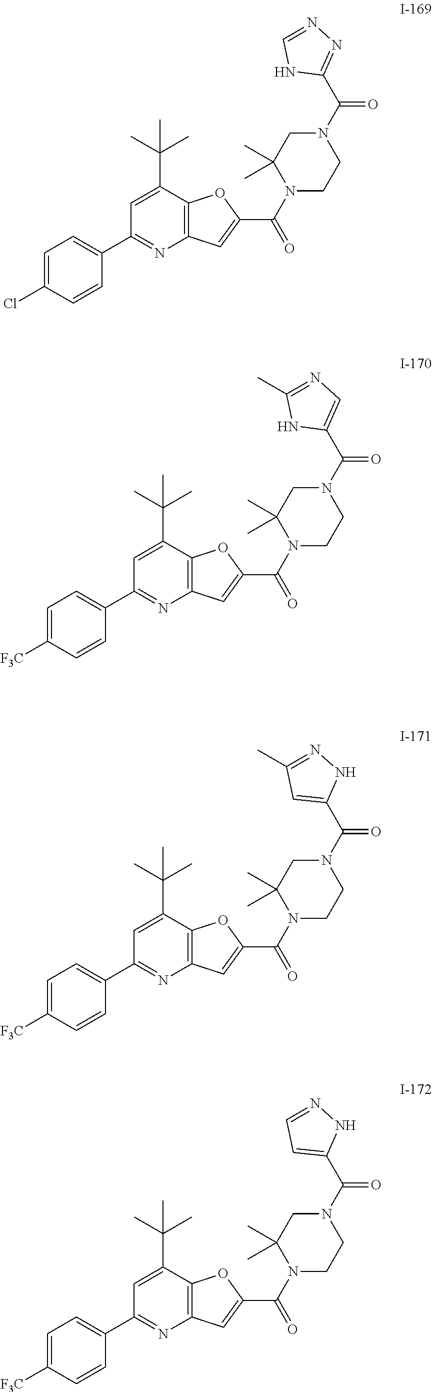 [SMILES: CC(C)(C)c1cc(-c2ccc(Cl)cc2)nc2cc(C(=O)N3CCN(C(=O)c4nnc[nH]4)CC3(C)C)oc12 ; Cc1ncc(C(=O)N2CCN(C(=O)c3cc4nc(-c5ccc(C(F)(F)F)cc5)cc(C(C)(C)C)c4o3)C(C)(C)C2)[nH]1 ; Cc1cc(C(=O)N2CCN(C(=O)c3cc4nc(-c5ccc(C(F)(F)F)cc5)cc(C(C)(C)C)c4o3)C(C)(C)C2)[nH]n1 ; CC(C)(C)c1cc(-c2ccc(C(F)(F)F)cc2)nc2cc(C(=O)N3CCN(C(=O)c4ccn[nH]4)CC3(C)C)oc12]

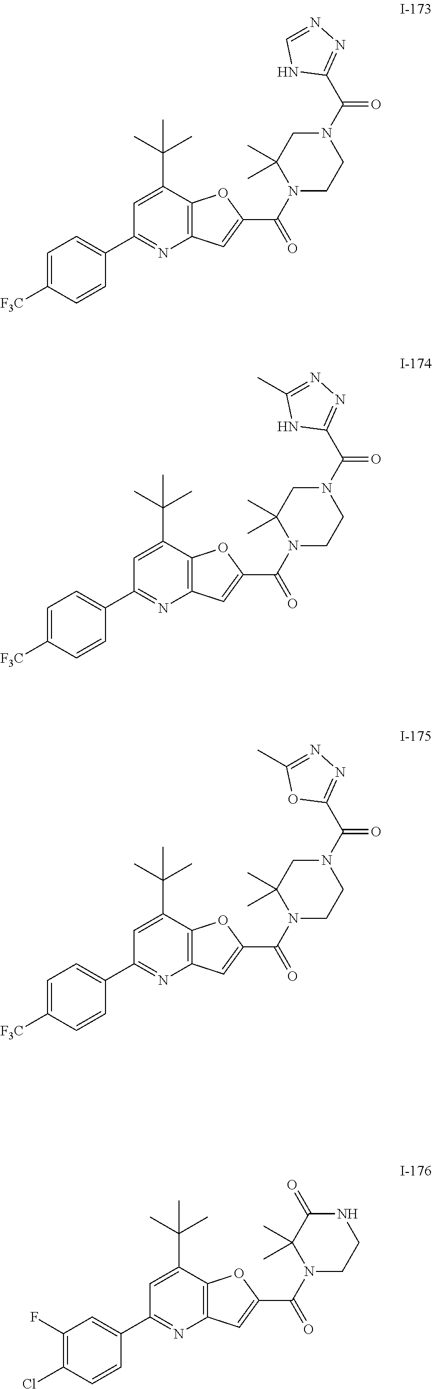 [SMILES: CC(C)(C)c1cc(-c2ccc(C(F)(F)F)cc2)nc2cc(C(=O)N3CCN(C(=O)c4nnc[nH]4)CC3(C)C)oc12 ; Cc1nnc(C(=O)N2CCN(C(=O)c3cc4nc(-c5ccc(C(F)(F)F)cc5)cc(C(C)(C)C)c4o3)C(C)(C)C2)[nH]1 ; Cc1nnc(C(=O)N2CCN(C(=O)c3cc4nc(-c5ccc(C(F)(F)F)cc5)cc(C(C)(C)C)c4o3)C(C)(C)C2)o1 ; CC(C)(C)c1cc(-c2ccc(Cl)c(F)c2)nc2cc(C(=O)N3CCNC(=O)C3(C)C)oc12]

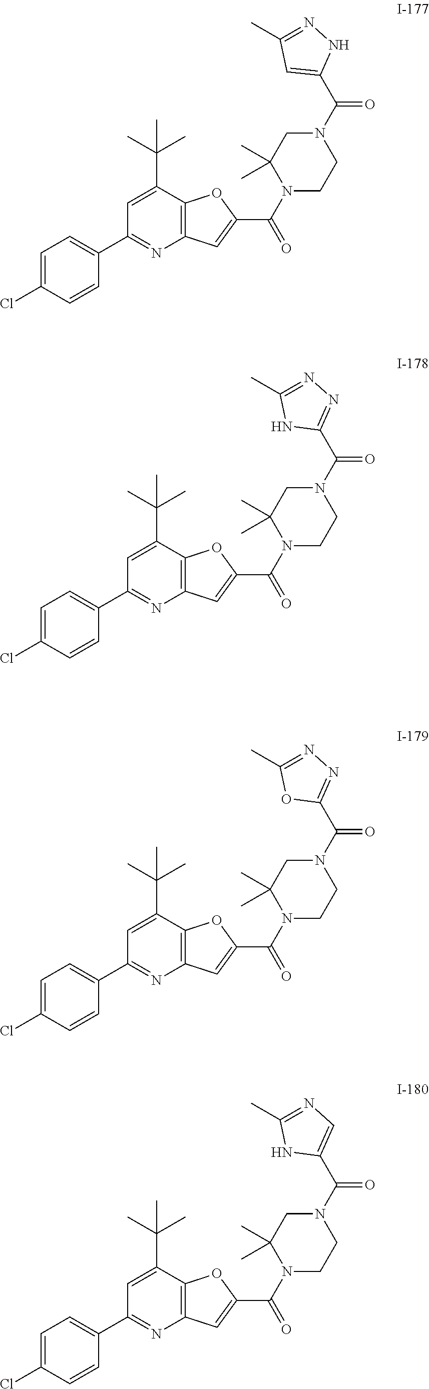 [SMILES: Cc1cc(C(=O)N2CCN(C(=O)c3cc4nc(-c5ccc(Cl)cc5)cc(C(C)(C)C)c4o3)C(C)(C)C2)[nH]n1 ; Cc1nnc(C(=O)N2CCN(C(=O)c3cc4nc(-c5ccc(Cl)cc5)cc(C(C)(C)C)c4o3)C(C)(C)C2)[nH]1 ; Cc1nnc(C(=O)N2CCN(C(=O)c3cc4nc(-c5ccc(Cl)cc5)cc(C(C)(C)C)c4o3)C(C)(C)C2)o1 ; Cc1ncc(C(=O)N2CCN(C(=O)c3cc4nc(-c5ccc(Cl)cc5)cc(C(C)(C)C)c4o3)C(C)(C)C2)[nH]1]

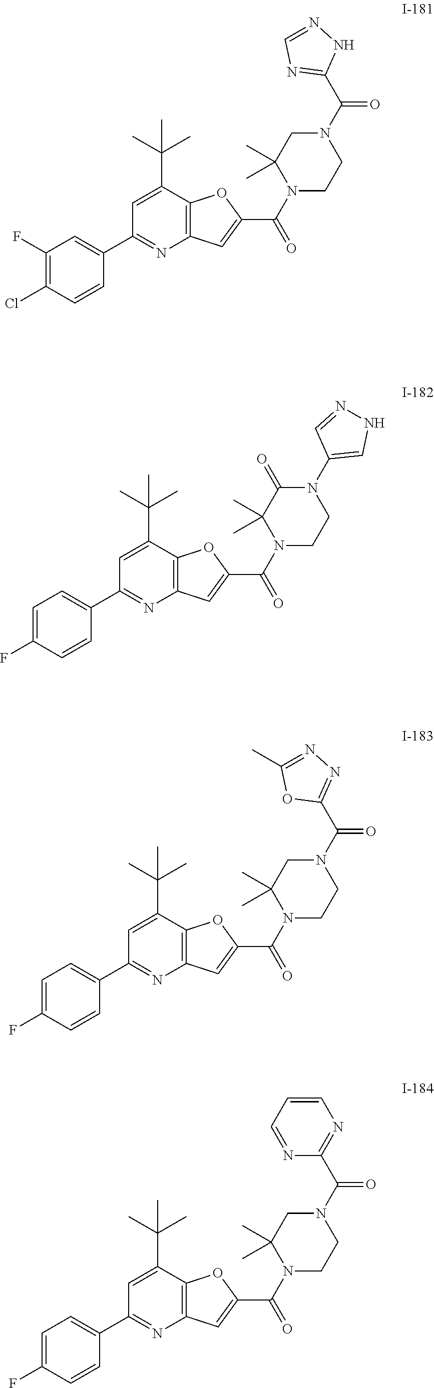 [SMILES: CC(C)(C)c1cc(-c2ccc(Cl)c(F)c2)nc2cc(C(=O)N3CCN(C(=O)c4ncn[nH]4)CC3(C)C)oc12 ; CC(C)(C)c1cc(-c2ccc(F)cc2)nc2cc(C(=O)N3CCN(c4cn[nH]c4)C(=O)C3(C)C)oc12 ; Cc1nnc(C(=O)N2CCN(C(=O)c3cc4nc(-c5ccc(F)cc5)cc(C(C)(C)C)c4o3)C(C)(C)C2)o1 ; CC(C)(C)c1cc(-c2ccc(F)cc2)nc2cc(C(=O)N3CCN(C(=O)c4ncccn4)CC3(C)C)oc12]

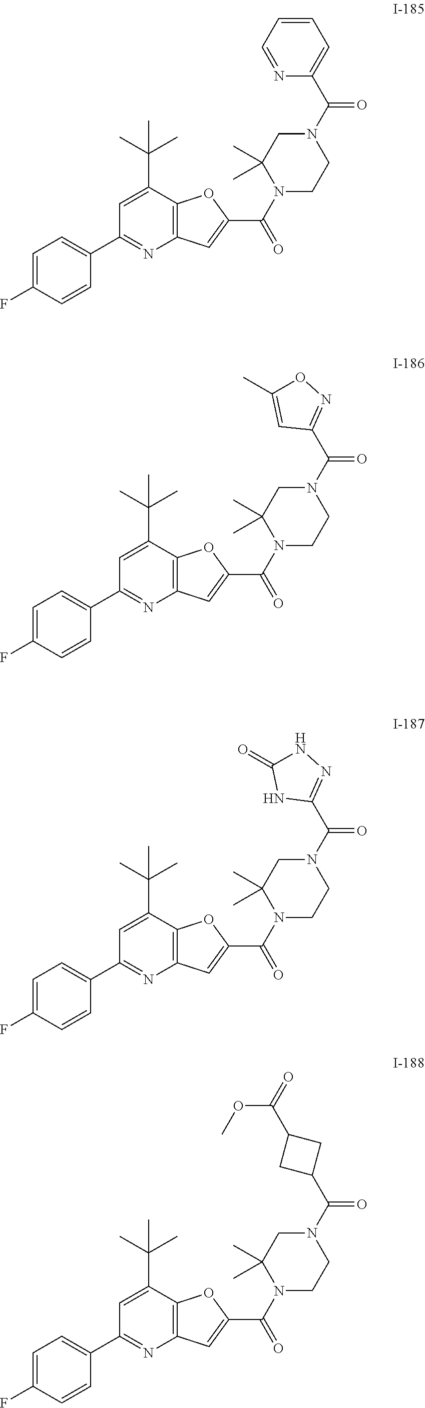 [SMILES: CC(C)(C)c1cc(-c2ccc(F)cc2)nc2cc(C(=O)N3CCN(C(=O)c4ccccn4)CC3(C)C)oc12 ; Cc1cc(C(=O)N2CCN(C(=O)c3cc4nc(-c5ccc(F)cc5)cc(C(C)(C)C)c4o3)C(C)(C)C2)no1 ; CC(C)(C)c1cc(-c2ccc(F)cc2)nc2cc(C(=O)N3CCN(C(=O)c4n[nH]c(=O)[nH]4)CC3(C)C)oc12 ; COC(=O)C1CC(C(=O)N2CCN(C(=O)c3cc4nc(-c5ccc(F)cc5)cc(C(C)(C)C)c4o3)C(C)(C)C2)C1]

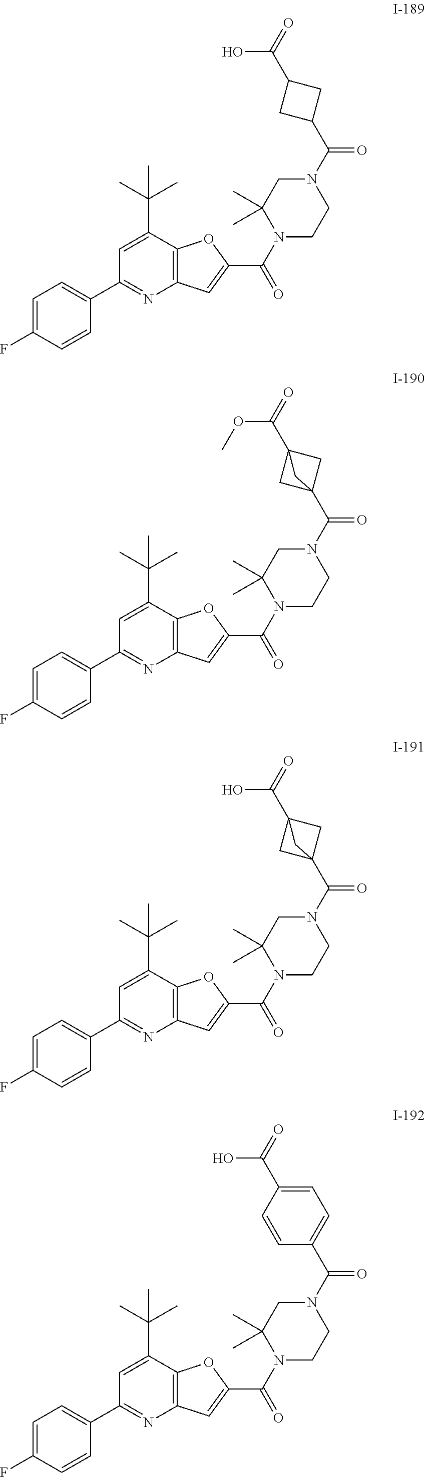 [SMILES: CC(C)(C)c1cc(-c2ccc(F)cc2)nc2cc(C(=O)N3CCN(C(=O)C4CC(C(=O)O)C4)CC3(C)C)oc12 ; COC(=O)C12CC(C(=O)N3CCN(C(=O)c4cc5nc(-c6ccc(F)cc6)cc(C(C)(C)C)c5o4)C(C)(C)C3)(C1)C2 ; CC(C)(C)c1cc(-c2ccc(F)cc2)nc2cc(C(=O)N3CCN(C(=O)C45CC(C(=O)O)(C4)C5)CC3(C)C)oc12 ; CC(C)(C)c1cc(-c2ccc(F)cc2)nc2cc(C(=O)N3CCN(C(=O)c4ccc(C(=O)O)cc4)CC3(C)C)oc12]

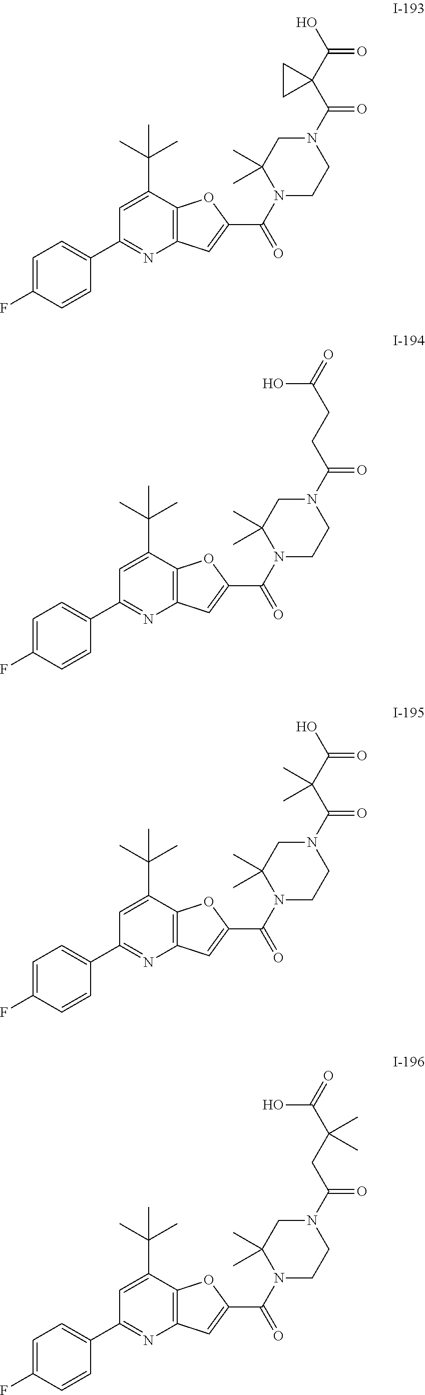 [SMILES: CC(C)(C)c1cc(-c2ccc(F)cc2)nc2cc(C(=O)N3CCN(C(=O)C4(C(=O)O)CC4)CC3(C)C)oc12 ; CC(C)(C)c1cc(-c2ccc(F)cc2)nc2cc(C(=O)N3CCN(C(=O)CCC(=O)O)CC3(C)C)oc12 ; CC(C)(C(=O)O)C(=O)N1CCN(C(=O)c2cc3nc(-c4ccc(F)cc4)cc(C(C)(C)C)c3o2)C(C)(C)C1 ; CC(C)(CC(=O)N1CCN(C(=O)c2cc3nc(-c4ccc(F)cc4)cc(C(C)(C)C)c3o2)C(C)(C)C1)C(=O)O]

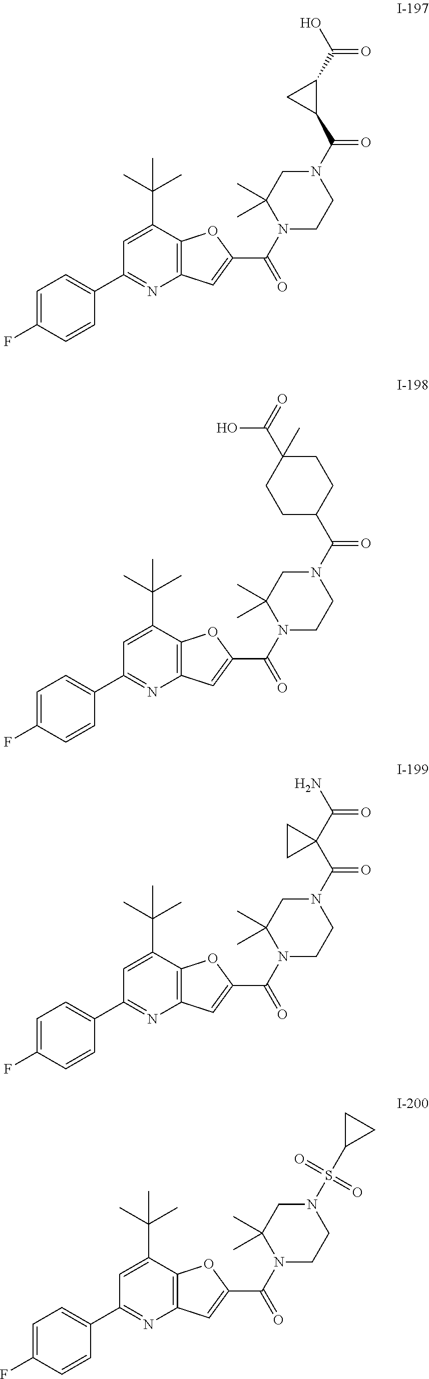 [SMILES: CC(C)(C)c1cc(-c2ccc(F)cc2)nc2cc(C(=O)N3CCN(C(=O)[C@H]4C[C@@H]4C(=O)O)CC3(C)C)oc12 ; CC1(C(=O)O)CCC(C(=O)N2CCN(C(=O)c3cc4nc(-c5ccc(F)cc5)cc(C(C)(C)C)c4o3)C(C)(C)C2)CC1 ; CC(C)(C)c1cc(-c2ccc(F)cc2)nc2cc(C(=O)N3CCN(C(=O)C4(C(N)=O)CC4)CC3(C)C)oc12 ; CC(C)(C)c1cc(-c2ccc(F)cc2)nc2cc(C(=O)N3CCN(S(=O)(=O)C4CC4)CC3(C)C)oc12]

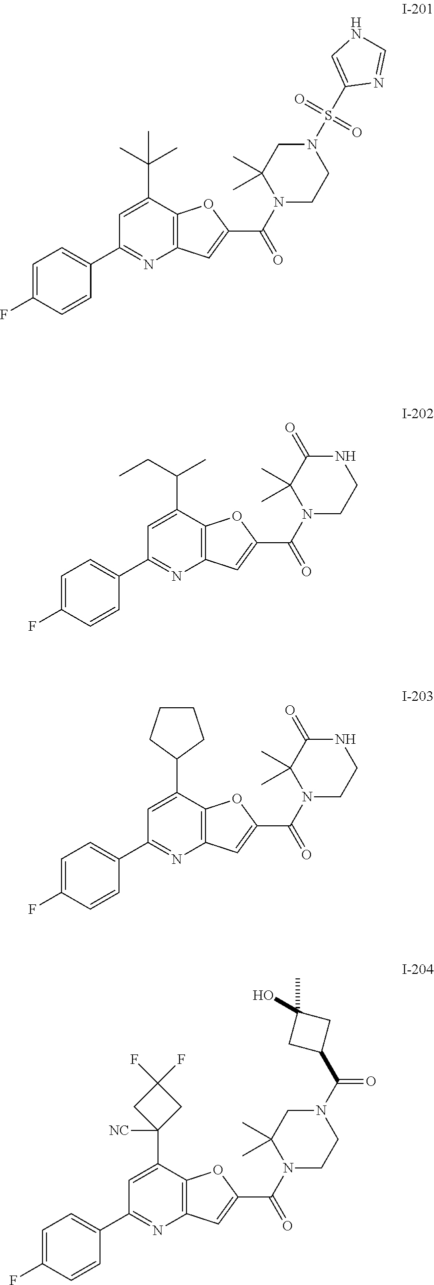

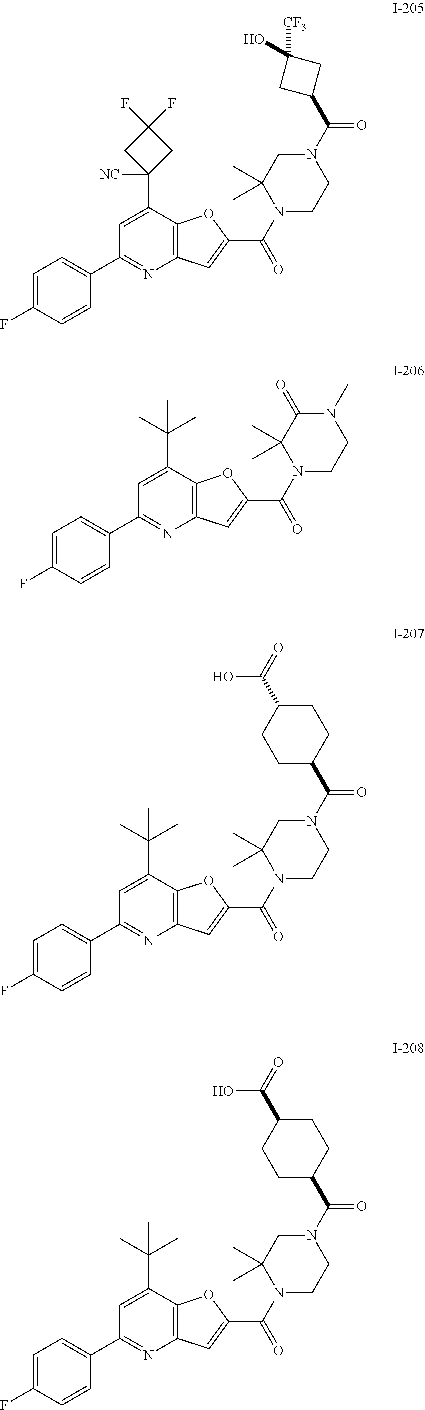

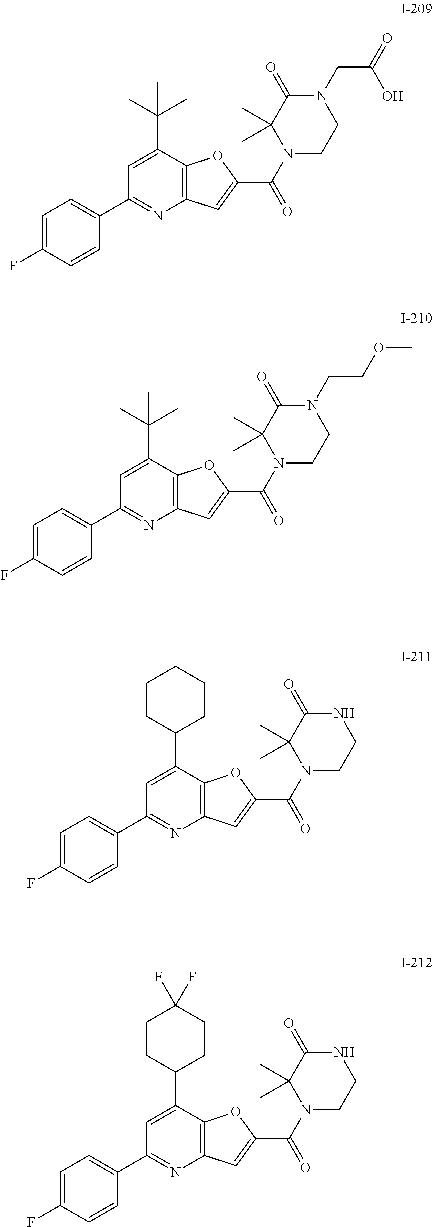 [SMILES: CC(C)(C)c1cc(-c2ccc(F)cc2)nc2cc(C(=O)N3CCN(CC(=O)O)C(=O)C3(C)C)oc12 ; COCCN1CCN(C(=O)c2cc3nc(-c4ccc(F)cc4)cc(C(C)(C)C)c3o2)C(C)(C)C1=O ; CC1(C)C(=O)NCCN1C(=O)c1cc2nc(-c3ccc(F)cc3)cc(C3CCCCC3)c2o1 ; CC1(C)C(=O)NCCN1C(=O)c1cc2nc(-c3ccc(F)cc3)cc(C3CCC(F)(F)CC3)c2o1]

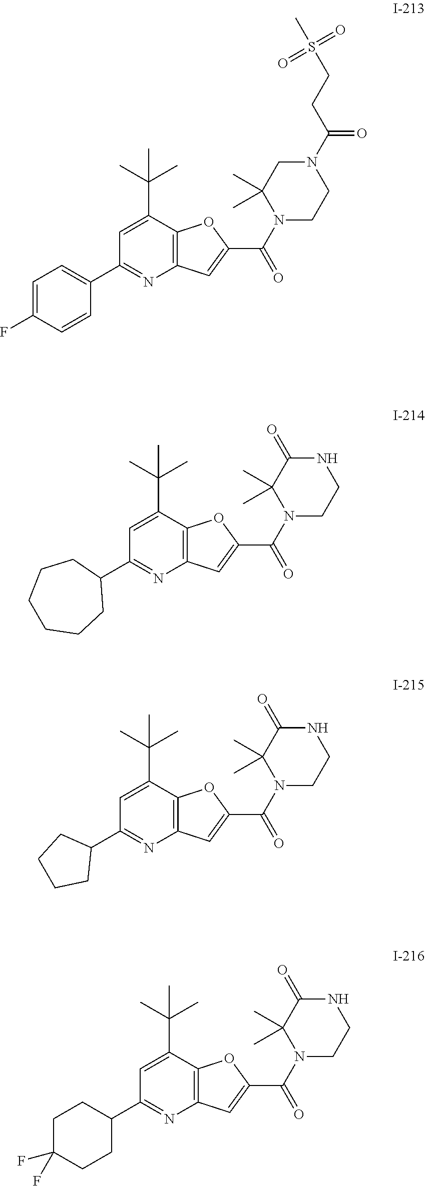 [SMILES: CC(C)(C)c1cc(-c2ccc(F)cc2)nc2cc(C(=O)N3CCN(C(=O)CCS(C)(=O)=O)CC3(C)C)oc12 ; CC(C)(C)c1cc(C2CCCCCC2)nc2cc(C(=O)N3CCNC(=O)C3(C)C)oc12 ; CC(C)(C)c1cc(C2CCCC2)nc2cc(C(=O)N3CCNC(=O)C3(C)C)oc12 ; CC(C)(C)c1cc(C2CCC(F)(F)CC2)nc2cc(C(=O)N3CCNC(=O)C3(C)C)oc12]

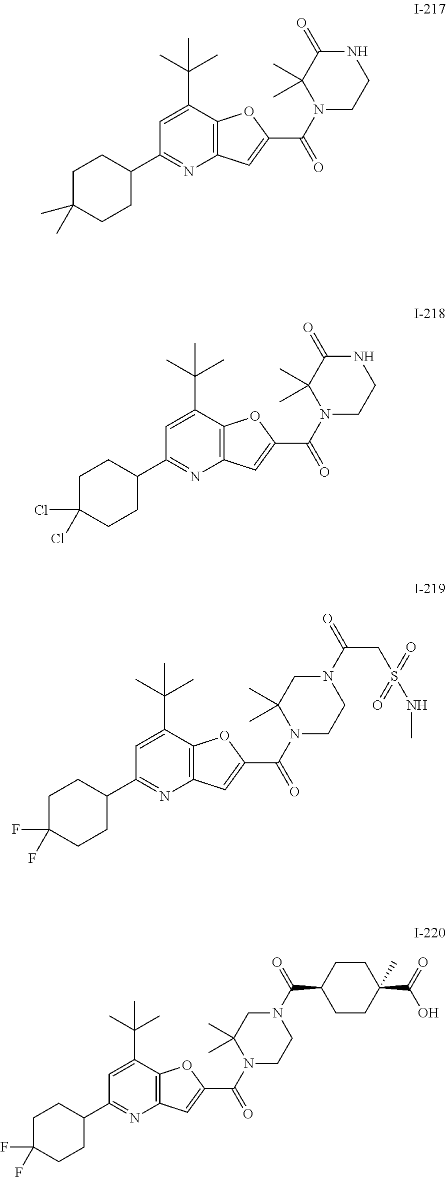 [SMILES: CC1(C)CCC(c2cc(C(C)(C)C)c3oc(C(=O)N4CCNC(=O)C4(C)C)cc3n2)CC1 ; CC(C)(C)c1cc(C2CCC(Cl)(Cl)CC2)nc2cc(C(=O)N3CCNC(=O)C3(C)C)oc12 ; CNS(=O)(=O)CC(=O)N1CCN(C(=O)c2cc3nc(C4CCC(F)(F)CC4)cc(C(C)(C)C)c3o2)C(C)(C)C1 ; CC(C)(C)c1cc(C2CCC(F)(F)CC2)nc2cc(C(=O)N3CCN(C(=O)[C@H]4CC[C@@](C)(C(=O)O)CC4)CC3(C)C)oc12]

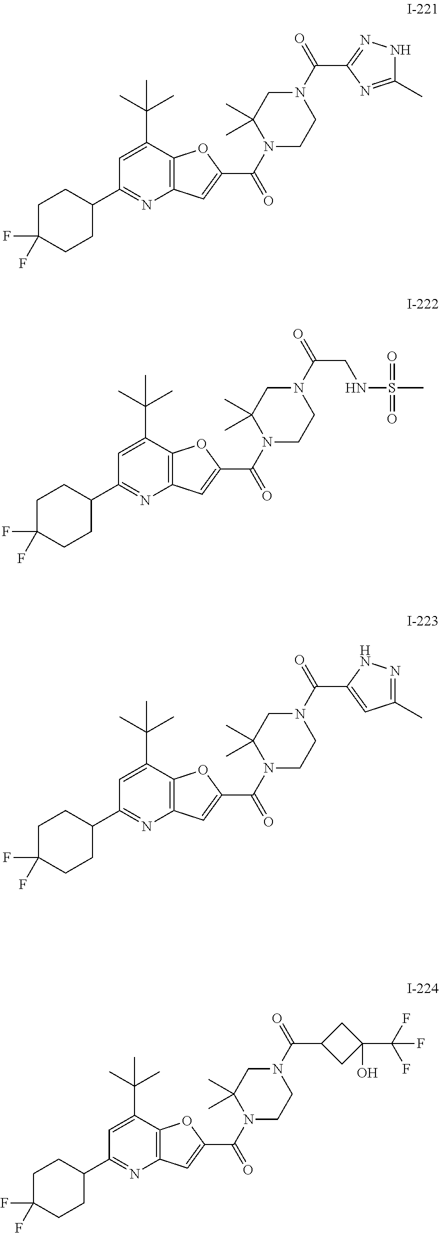 [SMILES: Cc1nc(C(=O)N2CCN(C(=O)c3cc4nc(C5CCC(F)(F)CC5)cc(C(C)(C)C)c4o3)C(C)(C)C2)n[nH]1 ; CC(C)(C)c1cc(C2CCC(F)(F)CC2)nc2cc(C(=O)N3CCN(C(=O)CNS(C)(=O)=O)CC3(C)C)oc12 ; Cc1cc(C(=O)N2CCN(C(=O)c3cc4nc(C5CCC(F)(F)CC5)cc(C(C)(C)C)c4o3)C(C)(C)C2)[nH]n1 ; CC(C)(C)c1cc(C2CCC(F)(F)CC2)nc2cc(C(=O)N3CCN(C(=O)C4CC(O)(C(F)(F)F)C4)CC3(C)C)oc12]

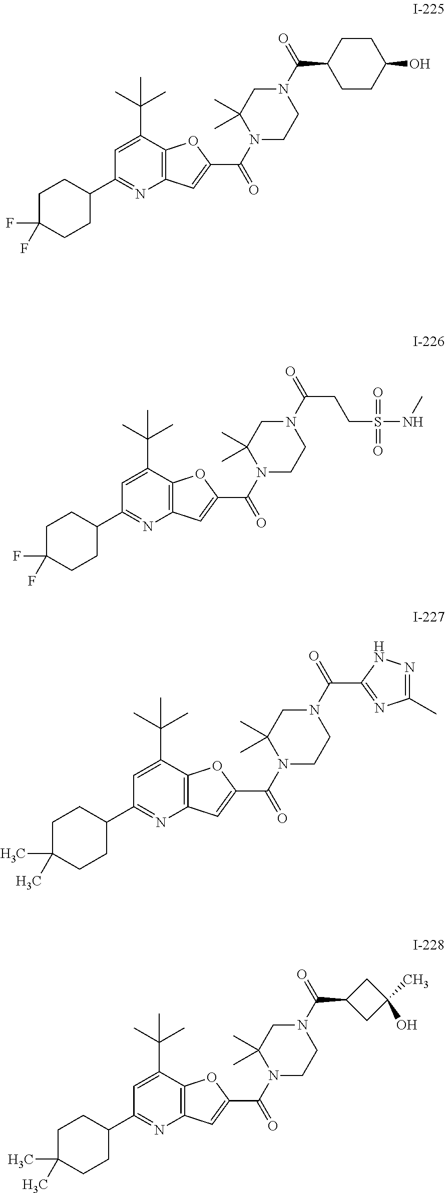

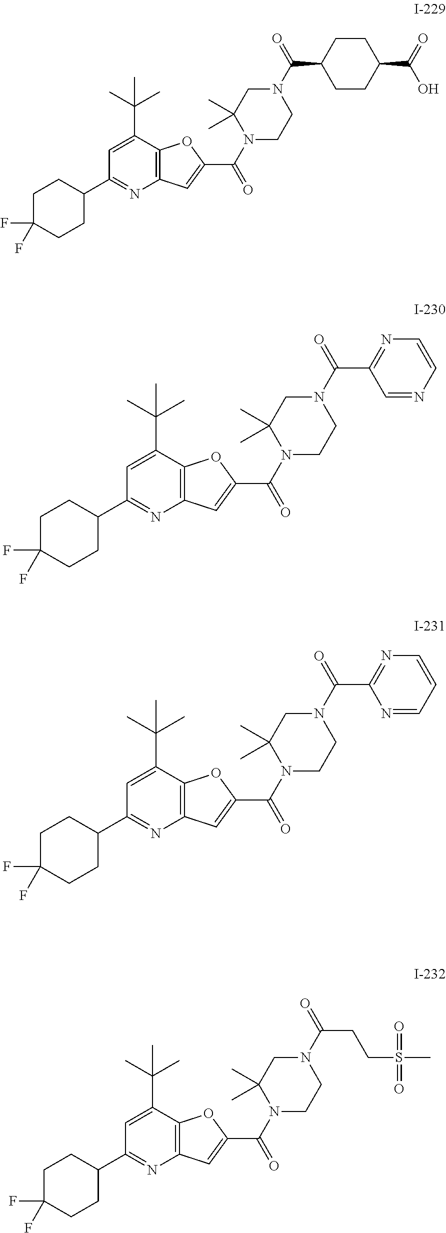 [SMILES: CC(C)(C)c1cc(C2CCC(F)(F)CC2)nc2cc(C(=O)N3CCN(C(=O)[C@H]4CC[C@@H](C(=O)O)CC4)CC3(C)C)oc12 ; CC(C)(C)c1cc(C2CCC(F)(F)CC2)nc2cc(C(=O)N3CCN(C(=O)c4cnccn4)CC3(C)C)oc12 ; CC(C)(C)c1cc(C2CCC(F)(F)CC2)nc2cc(C(=O)N3CCN(C(=O)c4ncccn4)CC3(C)C)oc12 ; CC(C)(C)c1cc(C2CCC(F)(F)CC2)nc2cc(C(=O)N3CCN(C(=O)CCS(C)(=O)=O)CC3(C)C)oc12]

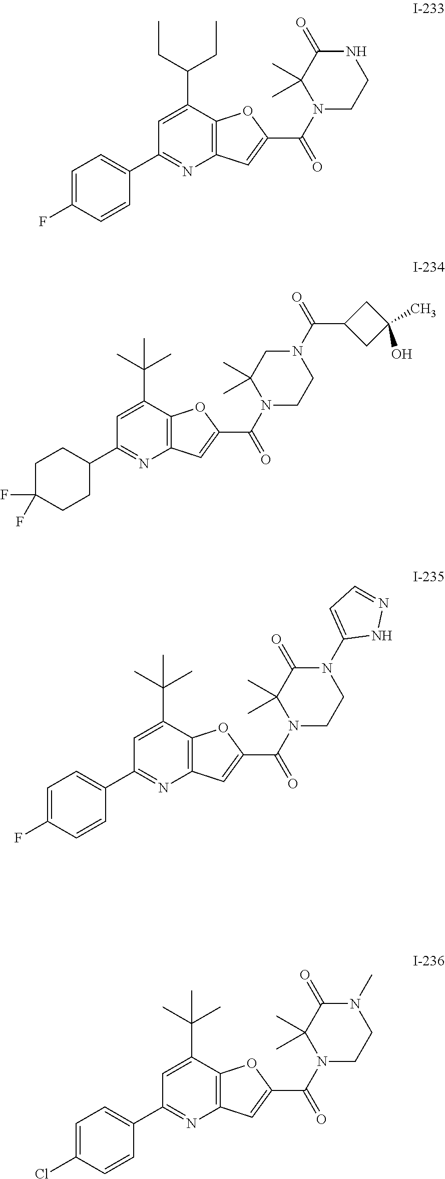

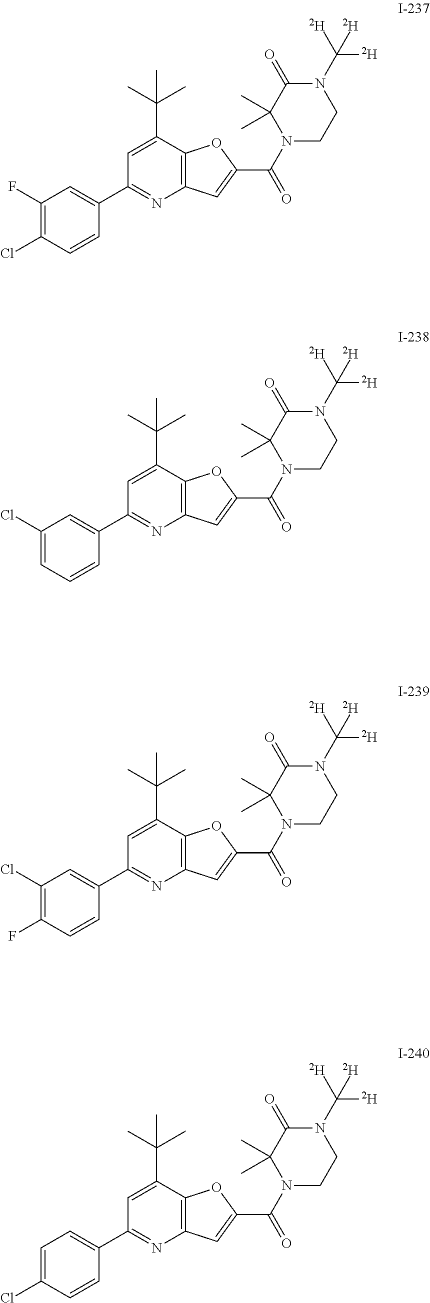 [SMILES: [2H]C([2H])([2H])N1CCN(C(=O)c2cc3nc(-c4ccc(Cl)c(F)c4)cc(C(C)(C)C)c3o2)C(C)(C)C1=O ; [2H]C([2H])([2H])N1CCN(C(=O)c2cc3nc(-c4cccc(Cl)c4)cc(C(C)(C)C)c3o2)C(C)(C)C1=O ; [2H]C([2H])([2H])N1CCN(C(=O)c2cc3nc(-c4ccc(F)c(Cl)c4)cc(C(C)(C)C)c3o2)C(C)(C)C1=O ; [2H]C([2H])([2H])N1CCN(C(=O)c2cc3nc(-c4ccc(Cl)cc4)cc(C(C)(C)C)c3o2)C(C)(C)C1=O]

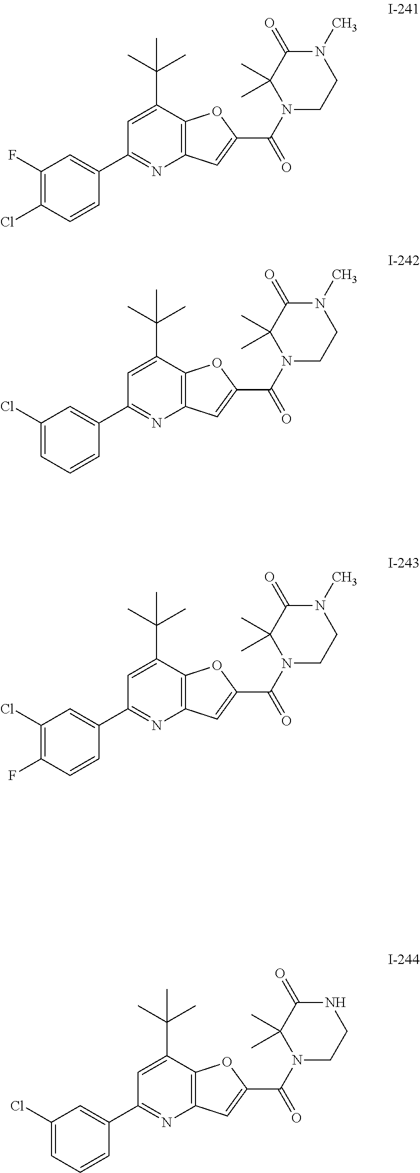 [SMILES: CN1CCN(C(=O)c2cc3nc(-c4ccc(Cl)c(F)c4)cc(C(C)(C)C)c3o2)C(C)(C)C1=O ; CN1CCN(C(=O)c2cc3nc(-c4cccc(Cl)c4)cc(C(C)(C)C)c3o2)C(C)(C)C1=O ; CN1CCN(C(=O)c2cc3nc(-c4ccc(F)c(Cl)c4)cc(C(C)(C)C)c3o2)C(C)(C)C1=O ; CC(C)(C)c1cc(-c2cccc(Cl)c2)nc2cc(C(=O)N3CCNC(=O)C3(C)C)oc12]

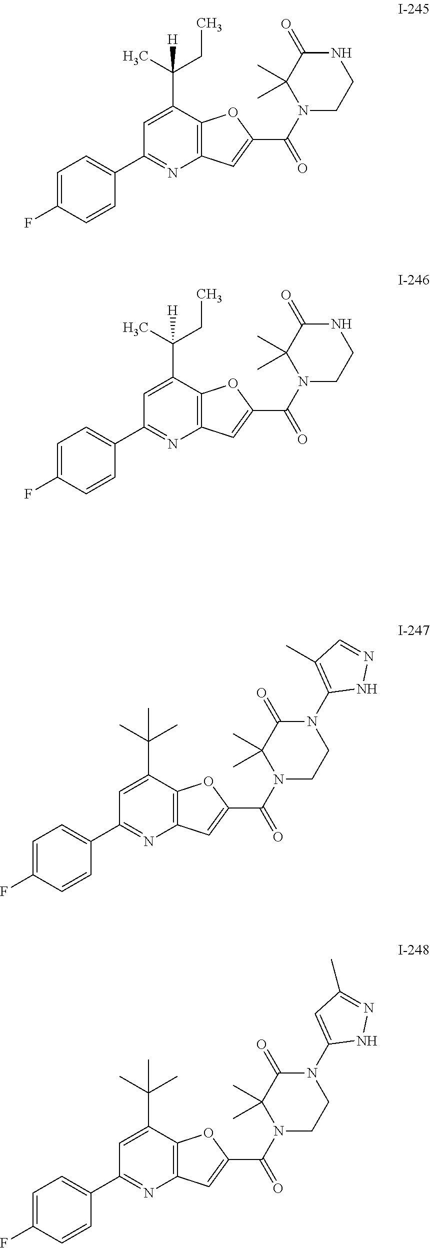 [SMILES: CC[C@@H](C)c1cc(-c2ccc(F)cc2)nc2cc(C(=O)N3CCNC(=O)C3(C)C)oc12 ; CC[C@H](C)c1cc(-c2ccc(F)cc2)nc2cc(C(=O)N3CCNC(=O)C3(C)C)oc12 ; Cc1cn[nH]c1N1CCN(C(=O)c2cc3nc(-c4ccc(F)cc4)cc(C(C)(C)C)c3o2)C(C)(C)C1=O ; Cc1cc(N2CCN(C(=O)c3cc4nc(-c5ccc(F)cc5)cc(C(C)(C)C)c4o3)C(C)(C)C2=O)[nH]n1]

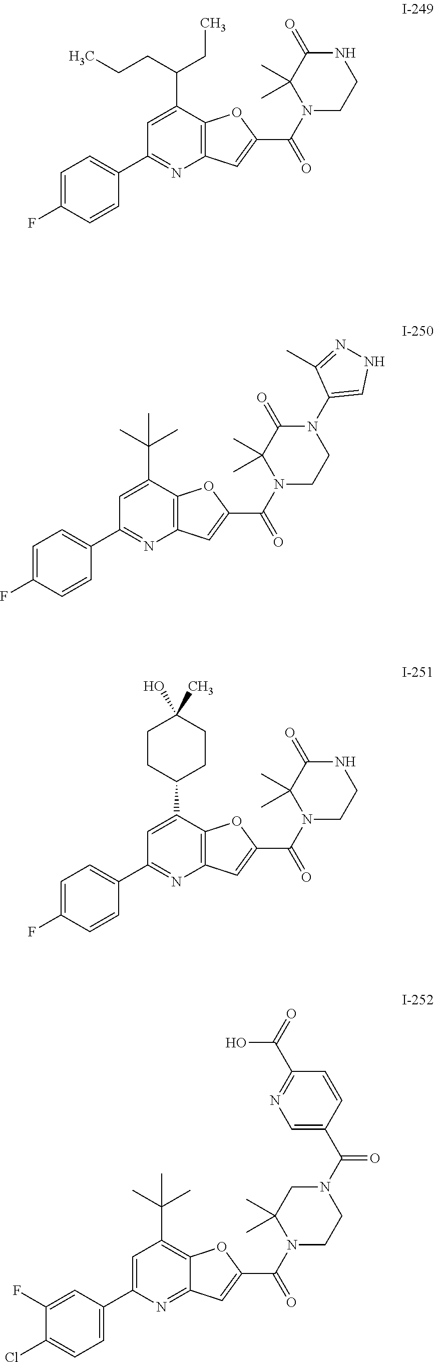 [SMILES: CCCC(CC)c1cc(-c2ccc(F)cc2)nc2cc(C(=O)N3CCNC(=O)C3(C)C)oc12 ; Cc1n[nH]cc1N1CCN(C(=O)c2cc3nc(-c4ccc(F)cc4)cc(C(C)(C)C)c3o2)C(C)(C)C1=O ; CC1(C)C(=O)NCCN1C(=O)c1cc2nc(-c3ccc(F)cc3)cc([C@H]3CC[C@@](C)(O)CC3)c2o1 ; CC(C)(C)c1cc(-c2ccc(Cl)c(F)c2)nc2cc(C(=O)N3CCN(C(=O)c4ccc(C(=O)O)nc4)CC3(C)C)oc12]

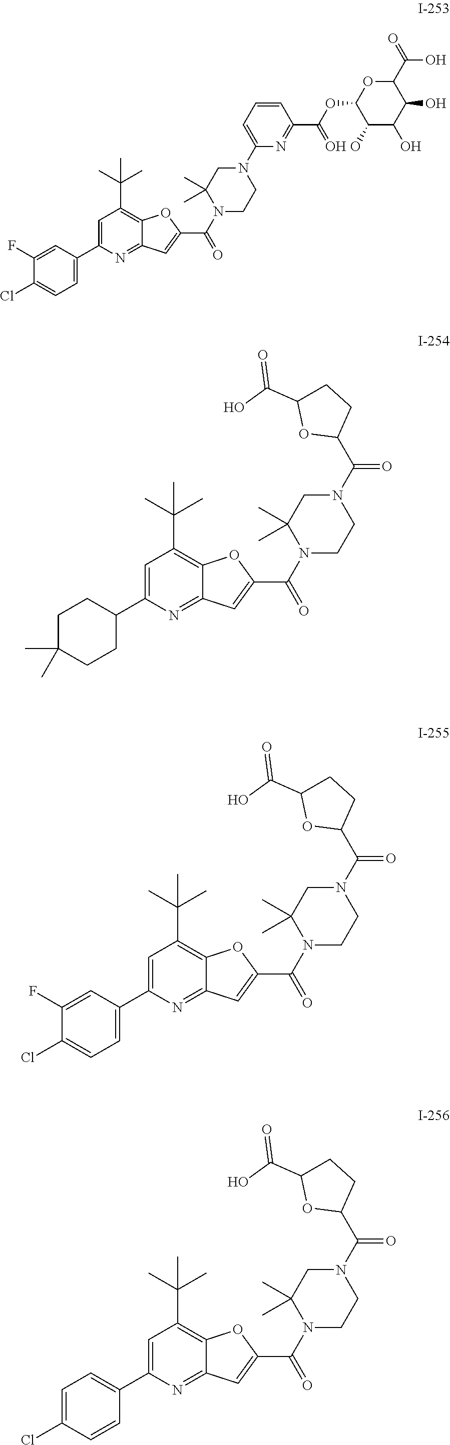 [SMILES: CC(C)(C)c1cc(-c2ccc(Cl)c(F)c2)nc2cc(C(=O)N3CCN(c4cccc(C(=O)O[C@@H]5OC(C(=O)O)[C@@H](O)C(O)[C@@H]5O)n4)CC3(C)C)oc12 ; CC1(C)CCC(c2cc(C(C)(C)C)c3oc(C(=O)N4CCN(C(=O)C5CCC(C(=O)O)O5)CC4(C)C)cc3n2)CC1 ; CC(C)(C)c1cc(-c2ccc(Cl)c(F)c2)nc2cc(C(=O)N3CCN(C(=O)C4CCC(C(=O)O)O4)CC3(C)C)oc12 ; CC(C)(C)c1cc(-c2ccc(Cl)cc2)nc2cc(C(=O)N3CCN(C(=O)C4CCC(C(=O)O)O4)CC3(C)C)oc12]

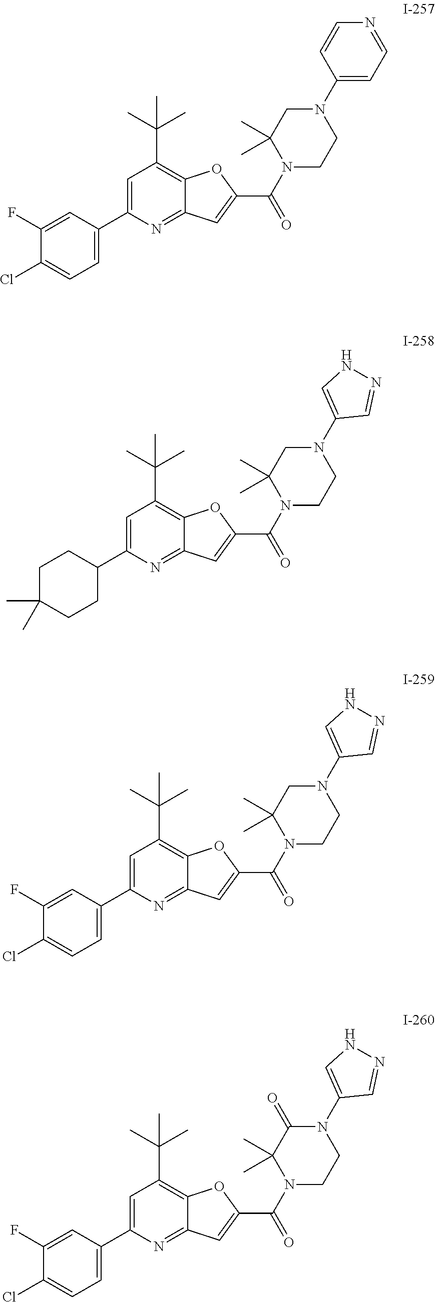 [SMILES: CC(C)(C)c1cc(-c2ccc(Cl)c(F)c2)nc2cc(C(=O)N3CCN(c4ccncc4)CC3(C)C)oc12 ; CC1(C)CCC(c2cc(C(C)(C)C)c3oc(C(=O)N4CCN(c5cn[nH]c5)CC4(C)C)cc3n2)CC1 ; CC(C)(C)c1cc(-c2ccc(Cl)c(F)c2)nc2cc(C(=O)N3CCN(c4cn[nH]c4)CC3(C)C)oc12 ; CC(C)(C)c1cc(-c2ccc(Cl)c(F)c2)nc2cc(C(=O)N3CCN(c4cn[nH]c4)C(=O)C3(C)C)oc12]

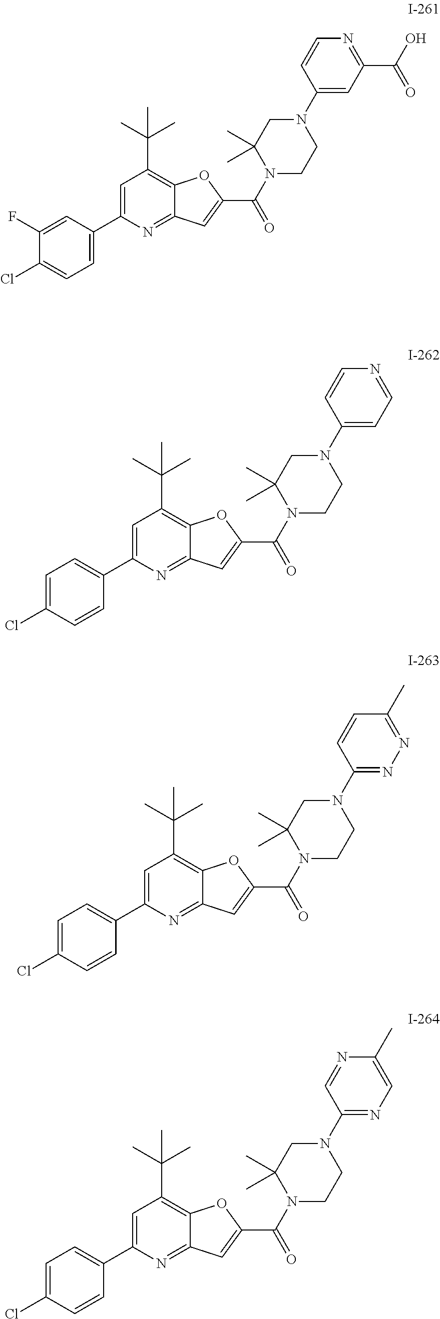 [SMILES: CC(C)(C)c1cc(-c2ccc(Cl)c(F)c2)nc2cc(C(=O)N3CCN(c4ccnc(C(=O)O)c4)CC3(C)C)oc12 ; CC(C)(C)c1cc(-c2ccc(Cl)cc2)nc2cc(C(=O)N3CCN(c4ccncc4)CC3(C)C)oc12 ; Cc1ccc(N2CCN(C(=O)c3cc4nc(-c5ccc(Cl)cc5)cc(C(C)(C)C)c4o3)C(C)(C)C2)nn1 ; Cc1cnc(N2CCN(C(=O)c3cc4nc(-c5ccc(Cl)cc5)cc(C(C)(C)C)c4o3)C(C)(C)C2)cn1]

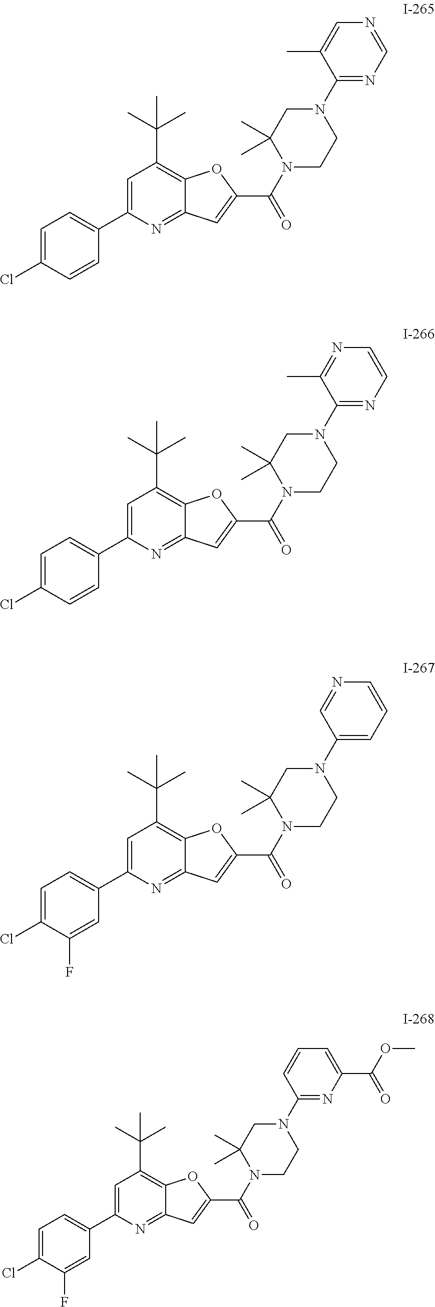 [SMILES: Cc1cncnc1N1CCN(C(=O)c2cc3nc(-c4ccc(Cl)cc4)cc(C(C)(C)C)c3o2)C(C)(C)C1 ; Cc1nccnc1N1CCN(C(=O)c2cc3nc(-c4ccc(Cl)cc4)cc(C(C)(C)C)c3o2)C(C)(C)C1 ; CC(C)(C)c1cc(-c2ccc(Cl)c(F)c2)nc2cc(C(=O)N3CCN(c4cccnc4)CC3(C)C)oc12 ; COC(=O)c1cccc(N2CCN(C(=O)c3cc4nc(-c5ccc(Cl)c(F)c5)cc(C(C)(C)C)c4o3)C(C)(C)C2)n1]

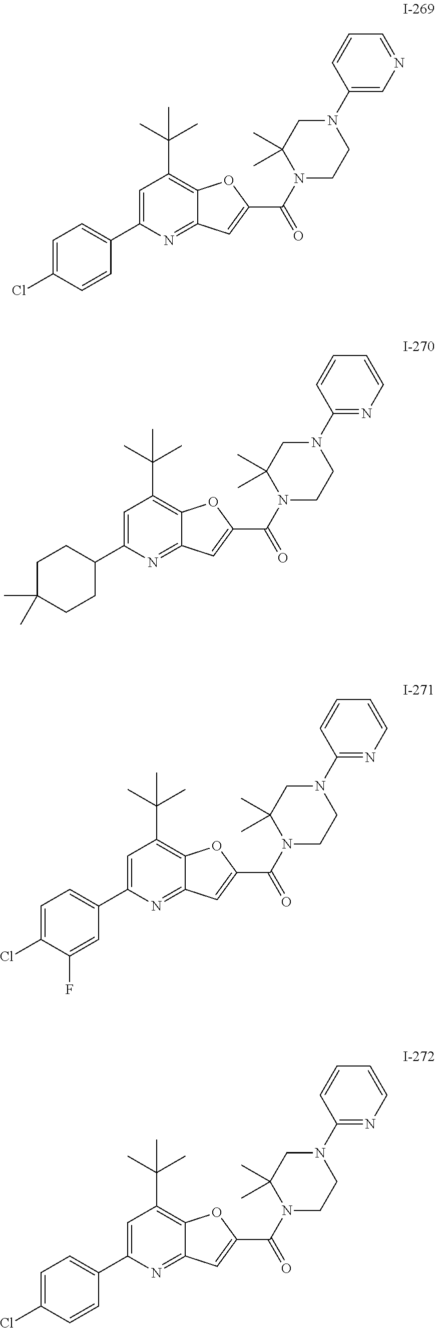 [SMILES: CC(C)(C)c1cc(-c2ccc(Cl)cc2)nc2cc(C(=O)N3CCN(c4cccnc4)CC3(C)C)oc12 ; CC1(C)CCC(c2cc(C(C)(C)C)c3oc(C(=O)N4CCN(c5ccccn5)CC4(C)C)cc3n2)CC1 ; CC(C)(C)c1cc(-c2ccc(Cl)c(F)c2)nc2cc(C(=O)N3CCN(c4ccccn4)CC3(C)C)oc12 ; CC(C)(C)c1cc(-c2ccc(Cl)cc2)nc2cc(C(=O)N3CCN(c4ccccn4)CC3(C)C)oc12]

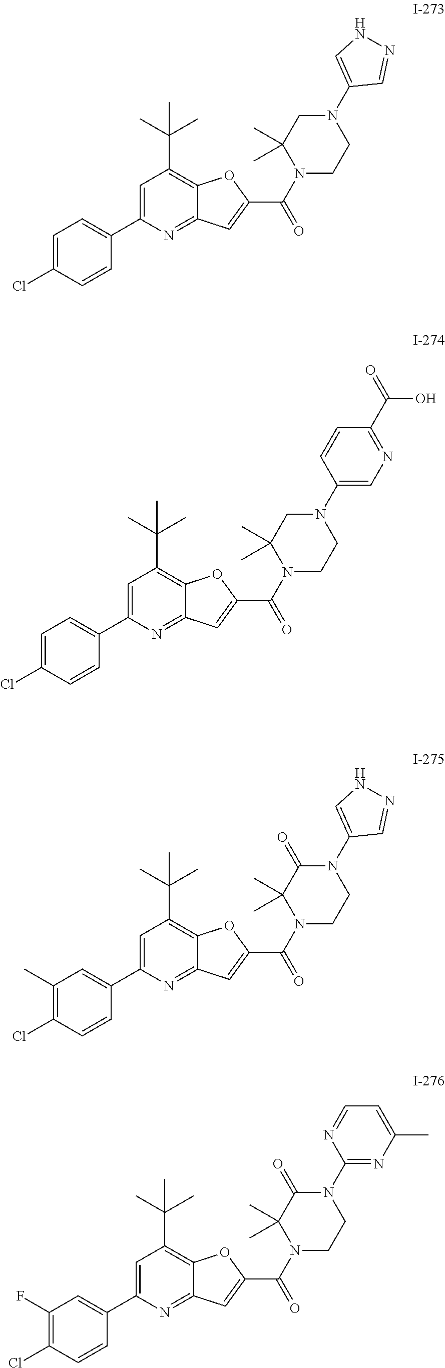 [SMILES: CC(C)(C)c1cc(-c2ccc(Cl)cc2)nc2cc(C(=O)N3CCN(c4cn[nH]c4)CC3(C)C)oc12 ; CC(C)(C)c1cc(-c2ccc(Cl)cc2)nc2cc(C(=O)N3CCN(c4ccc(C(=O)O)nc4)CC3(C)C)oc12 ; Cc1cc(-c2cc(C(C)(C)C)c3oc(C(=O)N4CCN(c5cn[nH]c5)C(=O)C4(C)C)cc3n2)ccc1Cl ; Cc1ccnc(N2CCN(C(=O)c3cc4nc(-c5ccc(Cl)c(F)c5)cc(C(C)(C)C)c4o3)C(C)(C)C2=O)n1]

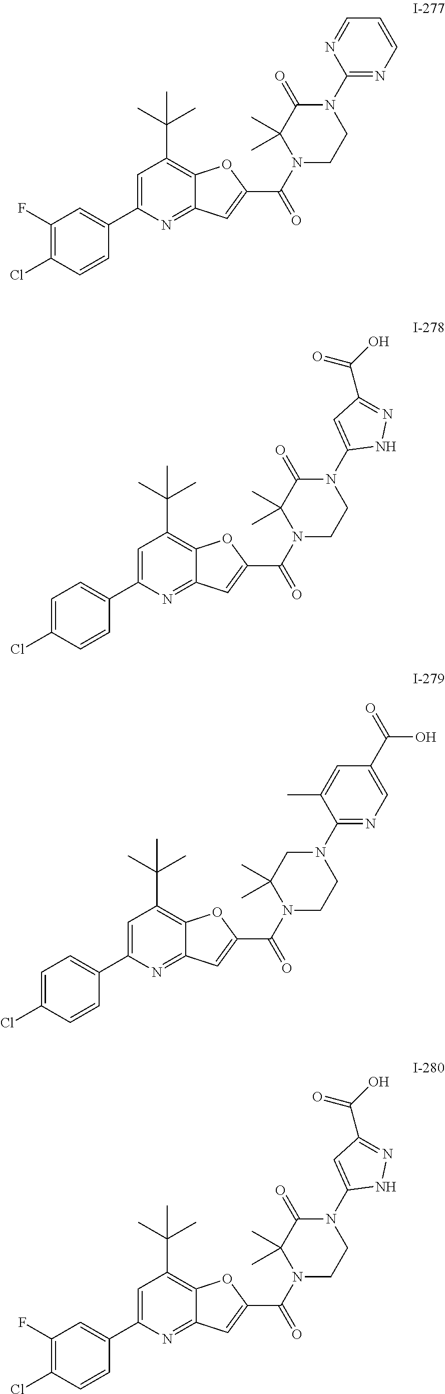 [SMILES: CC(C)(C)c1cc(-c2ccc(Cl)c(F)c2)nc2cc(C(=O)N3CCN(c4ncccn4)C(=O)C3(C)C)oc12 ; CC(C)(C)c1cc(-c2ccc(Cl)cc2)nc2cc(C(=O)N3CCN(c4cc(C(=O)O)n[nH]4)C(=O)C3(C)C)oc12 ; Cc1cc(C(=O)O)cnc1N1CCN(C(=O)c2cc3nc(-c4ccc(Cl)cc4)cc(C(C)(C)C)c3o2)C(C)(C)C1 ; CC(C)(C)c1cc(-c2ccc(Cl)c(F)c2)nc2cc(C(=O)N3CCN(c4cc(C(=O)O)n[nH]4)C(=O)C3(C)C)oc12]

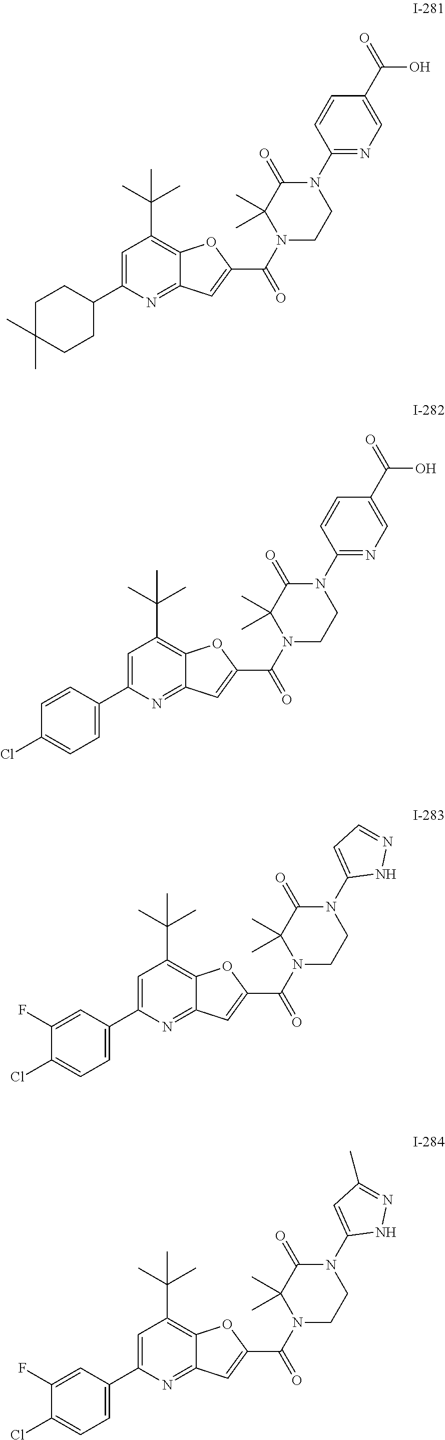 [SMILES: CC1(C)CCC(c2cc(C(C)(C)C)c3oc(C(=O)N4CCN(c5ccc(C(=O)O)cn5)C(=O)C4(C)C)cc3n2)CC1 ; CC(C)(C)c1cc(-c2ccc(Cl)cc2)nc2cc(C(=O)N3CCN(c4ccc(C(=O)O)cn4)C(=O)C3(C)C)oc12 ; CC(C)(C)c1cc(-c2ccc(Cl)c(F)c2)nc2cc(C(=O)N3CCN(c4ccn[nH]4)C(=O)C3(C)C)oc12 ; Cc1cc(N2CCN(C(=O)c3cc4nc(-c5ccc(Cl)c(F)c5)cc(C(C)(C)C)c4o3)C(C)(C)C2=O)[nH]n1]

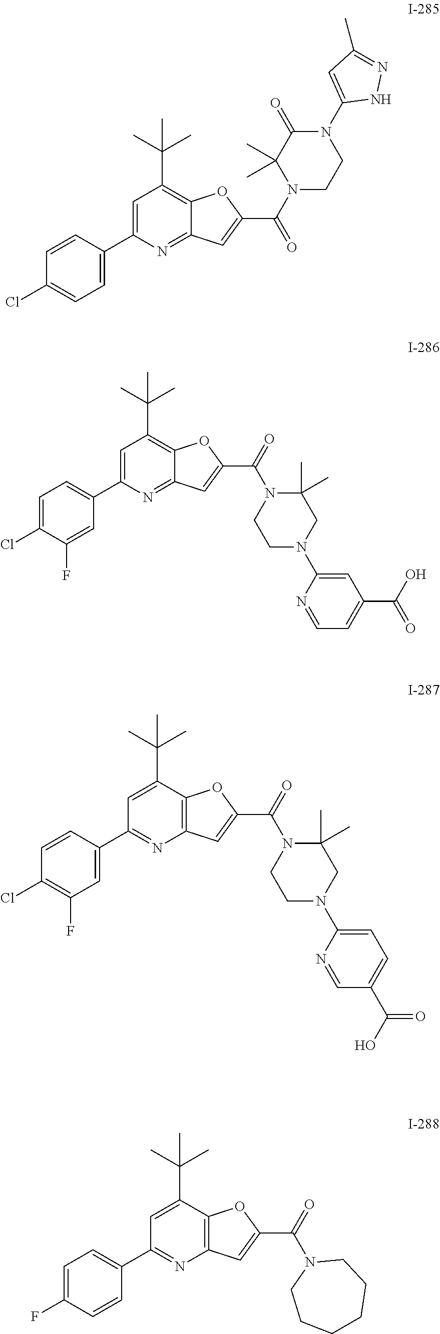 [SMILES: Cc1cc(N2CCN(C(=O)c3cc4nc(-c5ccc(Cl)cc5)cc(C(C)(C)C)c4o3)C(C)(C)C2=O)[nH]n1 ; CC(C)(C)c1cc(-c2ccc(Cl)c(F)c2)nc2cc(C(=O)N3CCN(c4cc(C(=O)O)ccn4)CC3(C)C)oc12 ; CC(C)(C)c1cc(-c2ccc(Cl)c(F)c2)nc2cc(C(=O)N3CCN(c4ccc(C(=O)O)cn4)CC3(C)C)oc12 ; CC(C)(C)c1cc(-c2ccc(F)cc2)nc2cc(C(=O)N3CCCCCC3)oc12]

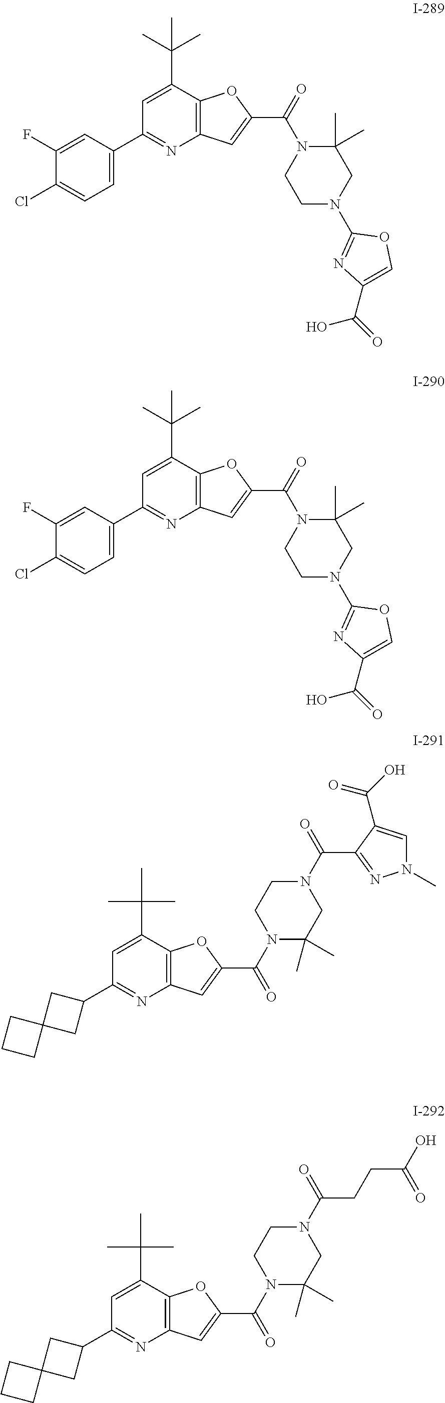 [SMILES: CC(C)(C)c1cc(-c2ccc(Cl)c(F)c2)nc2cc(C(=O)N3CCN(c4nc(C(=O)O)co4)CC3(C)C)oc12 ; CC(C)(C)c1cc(-c2ccc(Cl)c(F)c2)nc2cc(C(=O)N3CCN(c4nc(C(=O)O)co4)CC3(C)C)oc12 ; Cn1cc(C(=O)O)c(C(=O)N2CCN(C(=O)c3cc4nc(C5CC6(CCC6)C5)cc(C(C)(C)C)c4o3)C(C)(C)C2)n1 ; CC(C)(C)c1cc(C2CC3(CCC3)C2)nc2cc(C(=O)N3CCN(C(=O)CCC(=O)O)CC3(C)C)oc12]

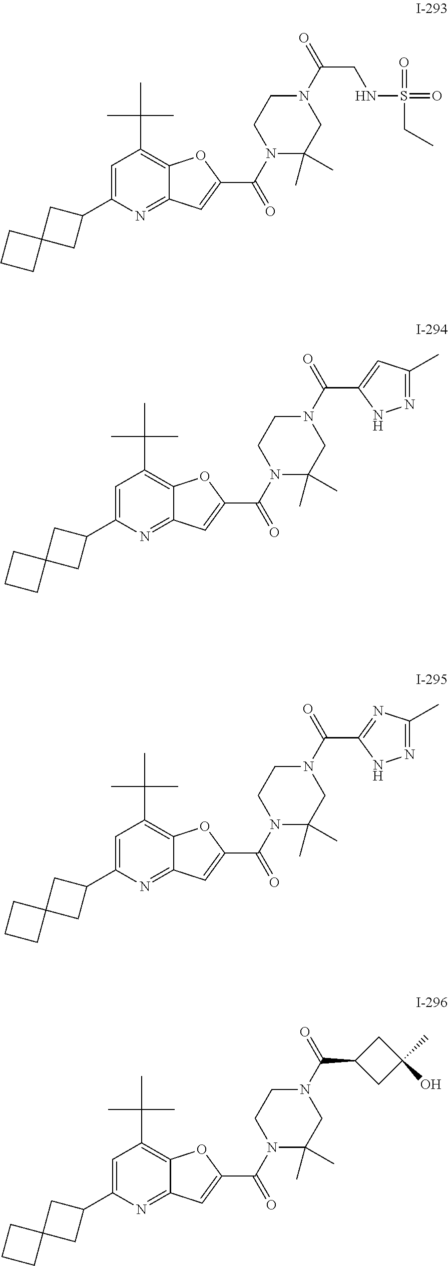 [SMILES: CCS(=O)(=O)NCC(=O)N1CCN(C(=O)c2cc3nc(C4CC5(CCC5)C4)cc(C(C)(C)C)c3o2)C(C)(C)C1 ; Cc1cc(C(=O)N2CCN(C(=O)c3cc4nc(C5CC6(CCC6)C5)cc(C(C)(C)C)c4o3)C(C)(C)C2)[nH]n1 ; Cc1n[nH]c(C(=O)N2CCN(C(=O)c3cc4nc(C5CC6(CCC6)C5)cc(C(C)(C)C)c4o3)C(C)(C)C2)n1 ; CC(C)(C)c1cc(C2CC3(CCC3)C2)nc2cc(C(=O)N3CCN(C(=O)[C@H]4C[C@@](C)(O)C4)CC3(C)C)oc12]

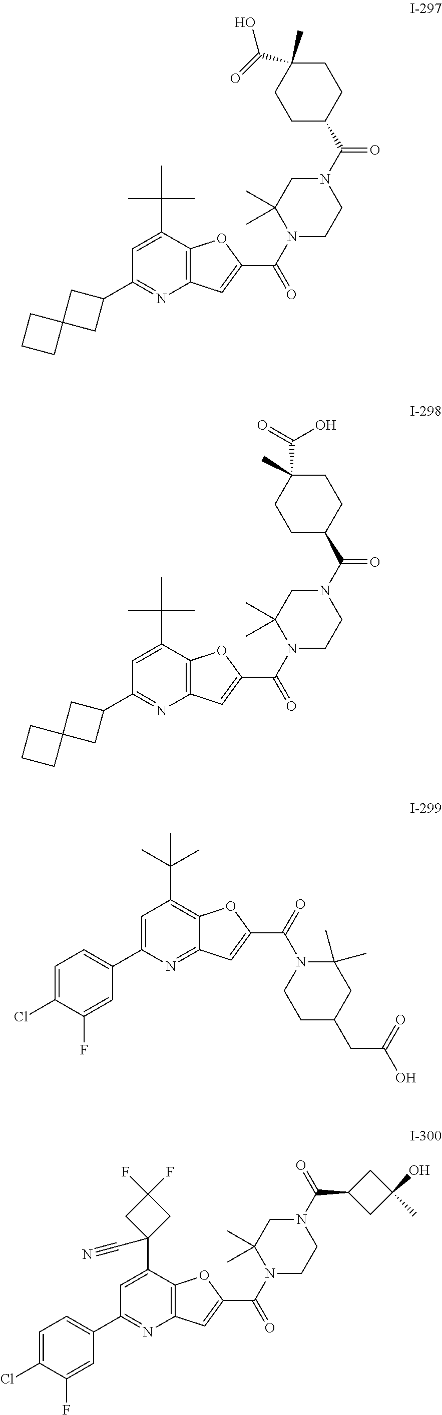 [SMILES: CC(C)(C)c1cc(C2CC3(CCC3)C2)nc2cc(C(=O)N3CCN(C(=O)[C@H]4CC[C@@](C)(C(=O)O)CC4)CC3(C)C)oc12 ; CC(C)(C)c1cc(C2CC3(CCC3)C2)nc2cc(C(=O)N3CCN(C(=O)[C@H]4CC[C@](C)(C(=O)O)CC4)CC3(C)C)oc12 ; CC(C)(C)c1cc(-c2ccc(Cl)c(F)c2)nc2cc(C(=O)N3CCC(CC(=O)O)CC3(C)C)oc12 ; CC1(C)CN(C(=O)[C@H]2C[C@@](C)(O)C2)CCN1C(=O)c1cc2nc(-c3ccc(Cl)c(F)c3)cc(C3(C#N)CC(F)(F)C3)c2o1]

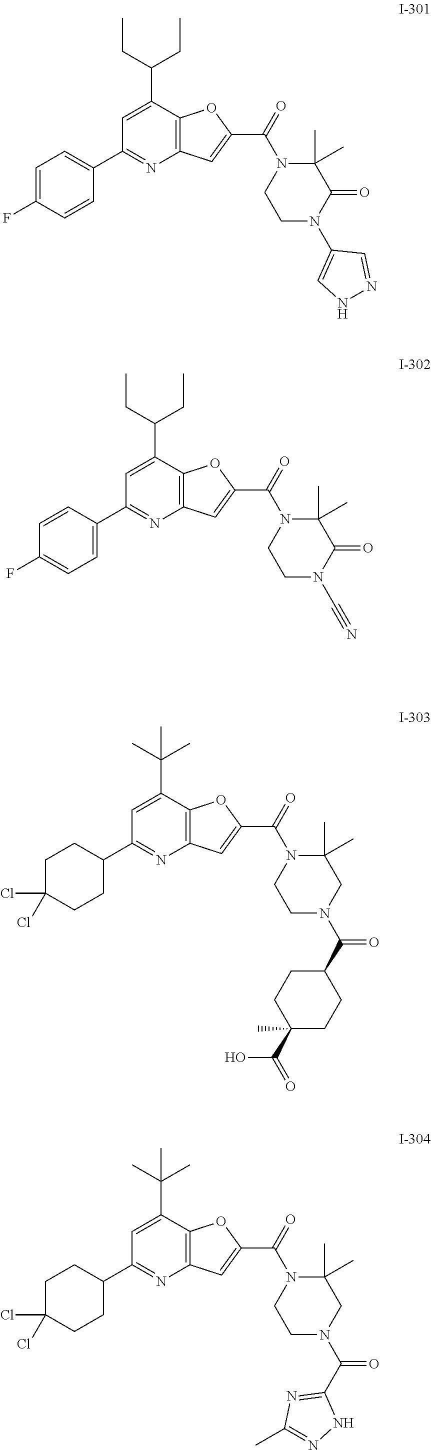 [SMILES: CCC(CC)c1cc(-c2ccc(F)cc2)nc2cc(C(=O)N3CCN(c4cn[nH]c4)C(=O)C3(C)C)oc12 ; CCC(CC)c1cc(-c2ccc(F)cc2)nc2cc(C(=O)N3CCN(C#N)C(=O)C3(C)C)oc12 ; CC(C)(C)c1cc(C2CCC(Cl)(Cl)CC2)nc2cc(C(=O)N3CCN(C(=O)[C@H]4CC[C@@](C)(C(=O)O)CC4)CC3(C)C)oc12 ; Cc1n[nH]c(C(=O)N2CCN(C(=O)c3cc4nc(C5CCC(Cl)(Cl)CC5)cc(C(C)(C)C)c4o3)C(C)(C)C2)n1]

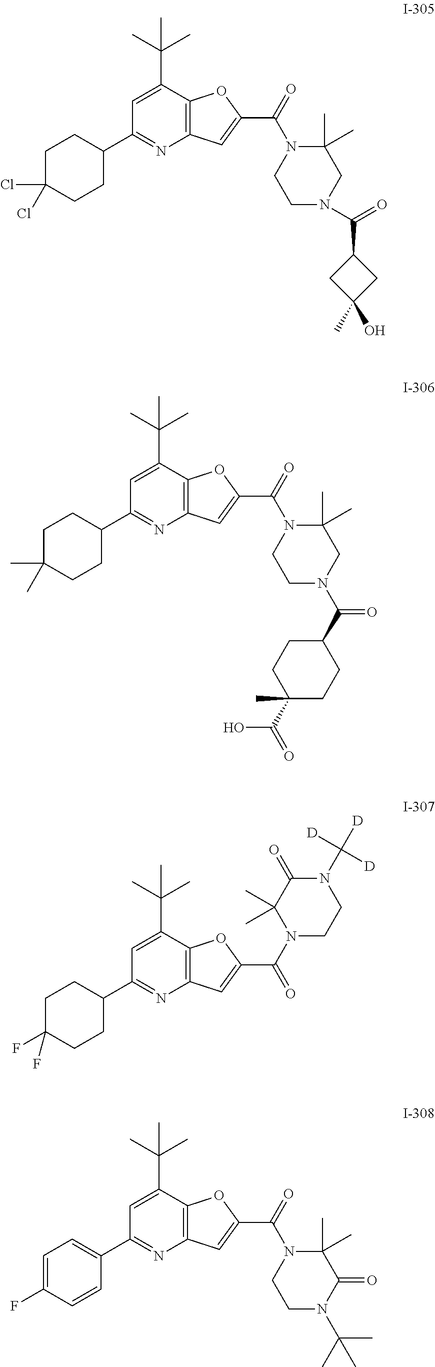 [SMILES: CC(C)(C)c1cc(C2CCC(Cl)(Cl)CC2)nc2cc(C(=O)N3CCN(C(=O)[C@H]4C[C@@](C)(O)C4)CC3(C)C)oc12 ; CC1(C)CCC(c2cc(C(C)(C)C)c3oc(C(=O)N4CCN(C(=O)[C@H]5CC[C@](C)(C(=O)O)CC5)CC4(C)C)cc3n2)CC1 ; [2H]C([2H])([2H])N1CCN(C(=O)c2cc3nc(C4CCC(F)(F)CC4)cc(C(C)(C)C)c3o2)C(C)(C)C1=O ; CC(C)(C)c1cc(-c2ccc(F)cc2)nc2cc(C(=O)N3CCN(C(C)(C)C)C(=O)C3(C)C)oc12]

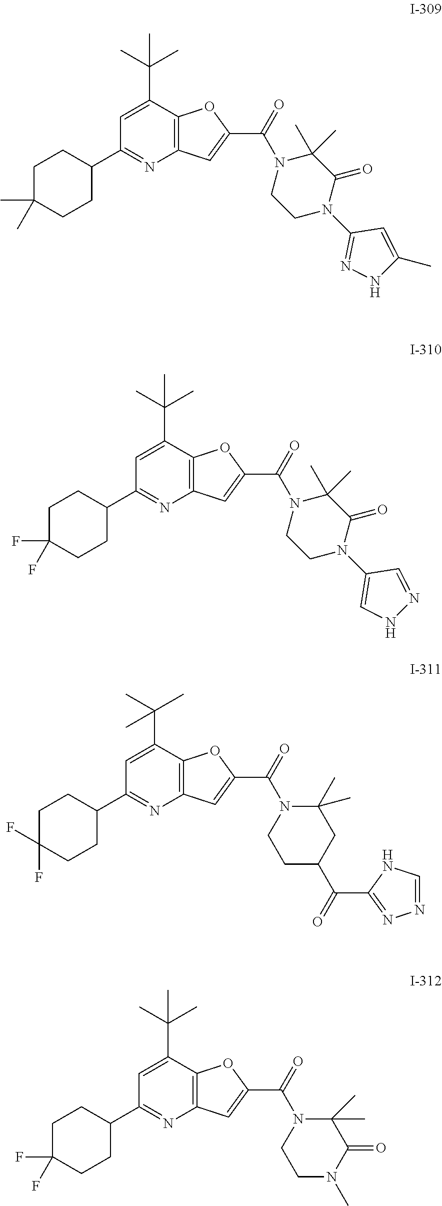

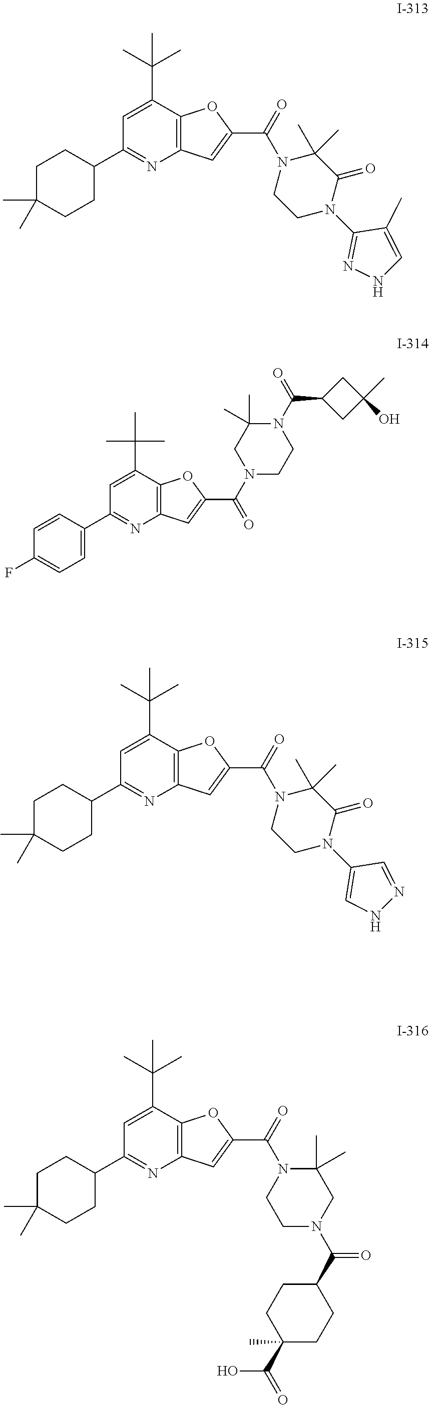

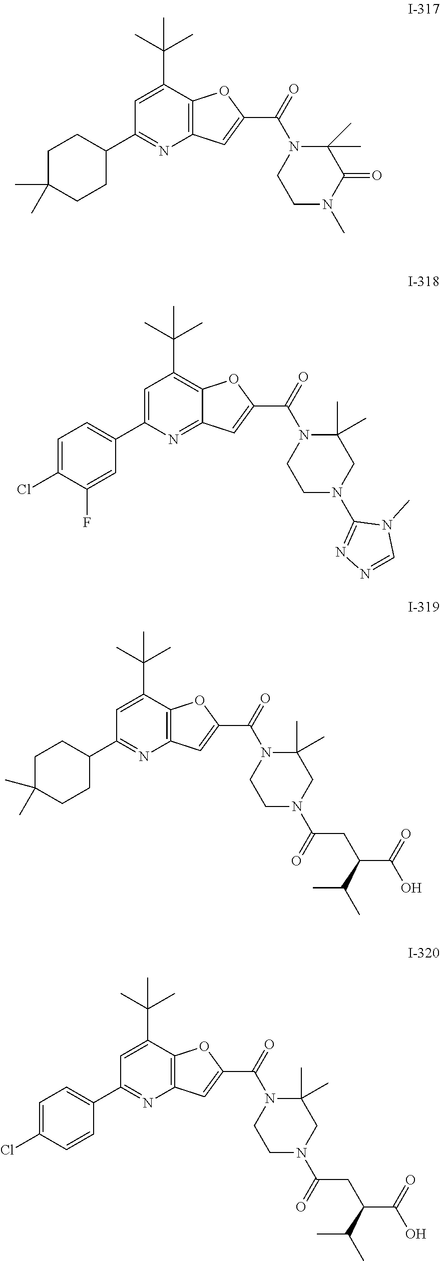

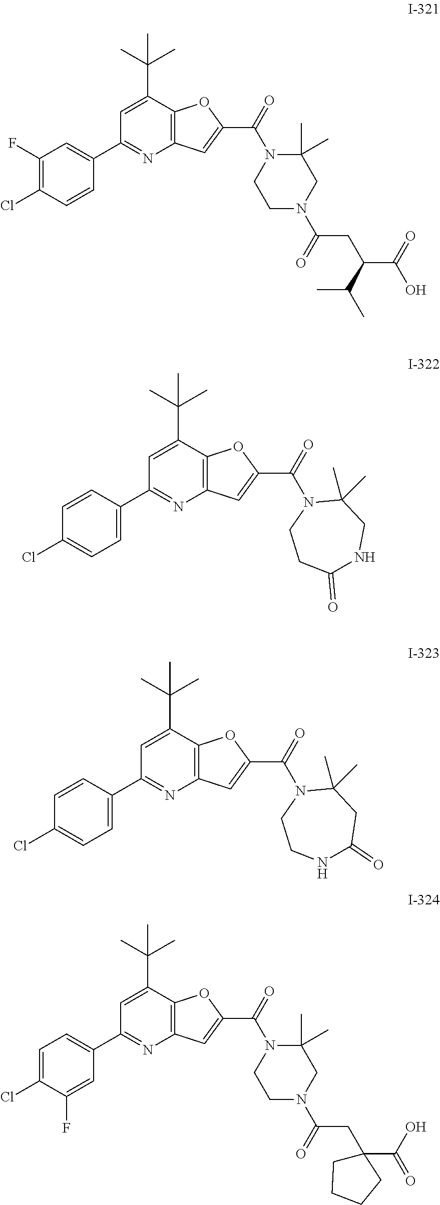 [SMILES: CC(C)[C@@H](CC(=O)N1CCN(C(=O)c2cc3nc(-c4ccc(Cl)c(F)c4)cc(C(C)(C)C)c3o2)C(C)(C)C1)C(=O)O ; CC(C)(C)c1cc(-c2ccc(Cl)cc2)nc2cc(C(=O)N3CCC(=O)NCC3(C)C)oc12 ; CC(C)(C)c1cc(-c2ccc(Cl)cc2)nc2cc(C(=O)N3CCNC(=O)CC3(C)C)oc12 ; CC(C)(C)c1cc(-c2ccc(Cl)c(F)c2)nc2cc(C(=O)N3CCN(C(=O)CC4(C(=O)O)CCCC4)CC3(C)C)oc12]

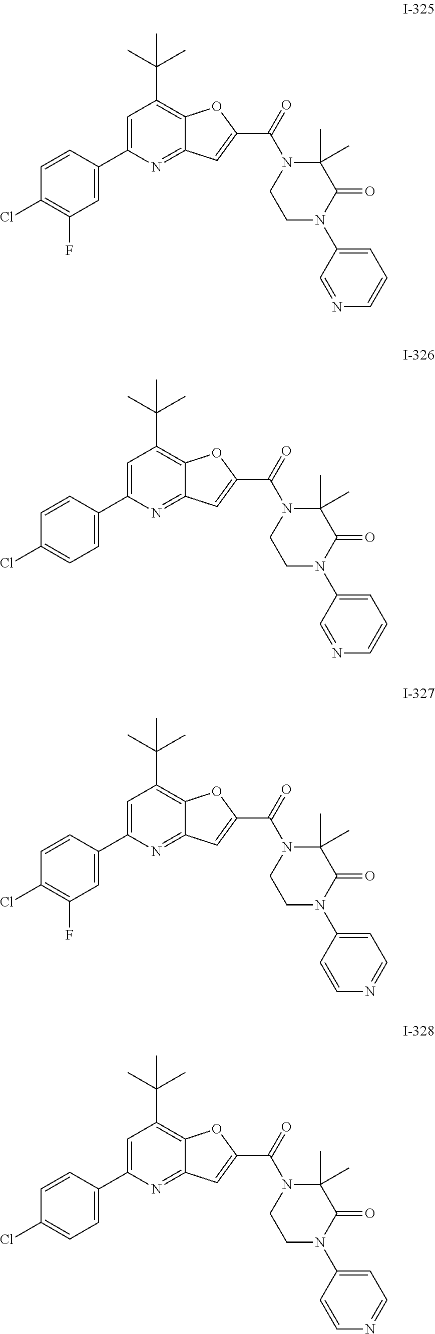 [SMILES: CC(C)(C)c1cc(-c2ccc(Cl)c(F)c2)nc2cc(C(=O)N3CCN(c4cccnc4)C(=O)C3(C)C)oc12 ; CC(C)(C)c1cc(-c2ccc(Cl)cc2)nc2cc(C(=O)N3CCN(c4cccnc4)C(=O)C3(C)C)oc12 ; CC(C)(C)c1cc(-c2ccc(Cl)c(F)c2)nc2cc(C(=O)N3CCN(c4ccncc4)C(=O)C3(C)C)oc12 ; CC(C)(C)c1cc(-c2ccc(Cl)cc2)nc2cc(C(=O)N3CCN(c4ccncc4)C(=O)C3(C)C)oc12]

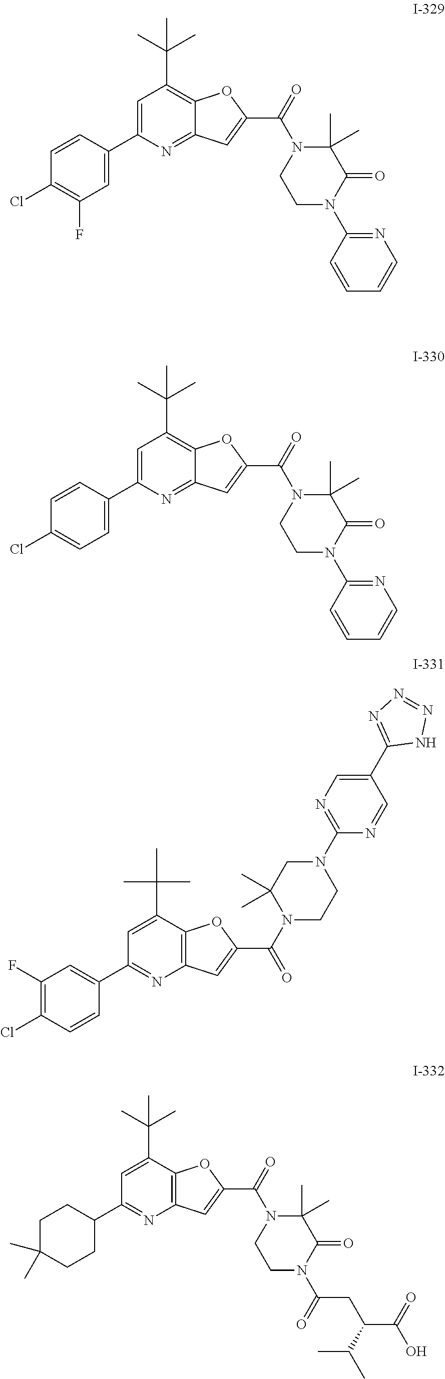 [SMILES: CC(C)(C)c1cc(-c2ccc(Cl)c(F)c2)nc2cc(C(=O)N3CCN(c4ccccn4)C(=O)C3(C)C)oc12 ; CC(C)(C)c1cc(-c2ccc(Cl)cc2)nc2cc(C(=O)N3CCN(c4ccccn4)C(=O)C3(C)C)oc12 ; CC(C)(C)c1cc(-c2ccc(Cl)c(F)c2)nc2cc(C(=O)N3CCN(c4ncc(-c5nnn[nH]5)cn4)CC3(C)C)oc12 ; CC(C)[C@H](CC(=O)N1CCN(C(=O)c2cc3nc(C4CCC(C)(C)CC4)cc(C(C)(C)C)c3o2)C(C)(C)C1=O)C(=O)O]

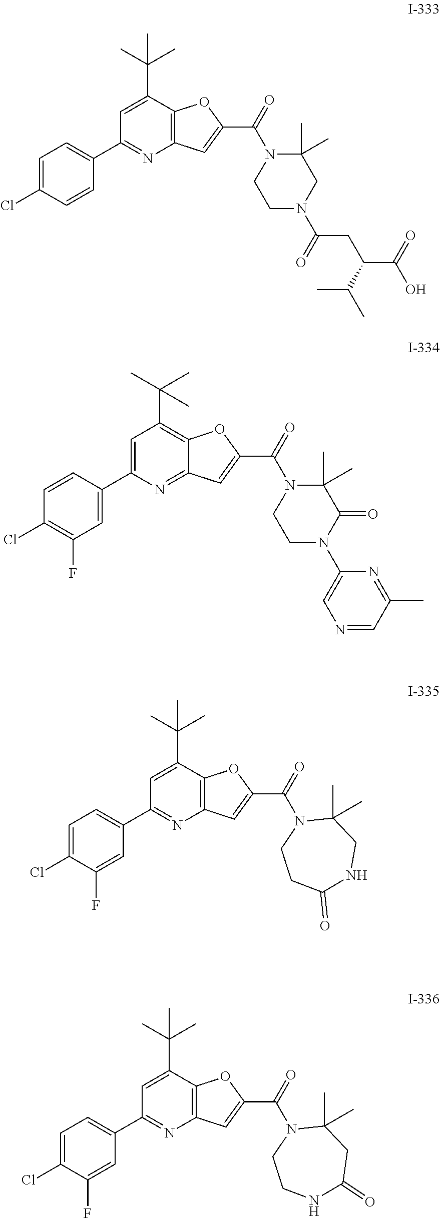 [SMILES: CC(C)[C@H](CC(=O)N1CCN(C(=O)c2cc3nc(-c4ccc(Cl)cc4)cc(C(C)(C)C)c3o2)C(C)(C)C1)C(=O)O ; Cc1cncc(N2CCN(C(=O)c3cc4nc(-c5ccc(Cl)c(F)c5)cc(C(C)(C)C)c4o3)C(C)(C)C2=O)n1 ; CC(C)(C)c1cc(-c2ccc(Cl)c(F)c2)nc2cc(C(=O)N3CCC(=O)NCC3(C)C)oc12 ; CC(C)(C)c1cc(-c2ccc(Cl)c(F)c2)nc2cc(C(=O)N3CCNC(=O)CC3(C)C)oc12]

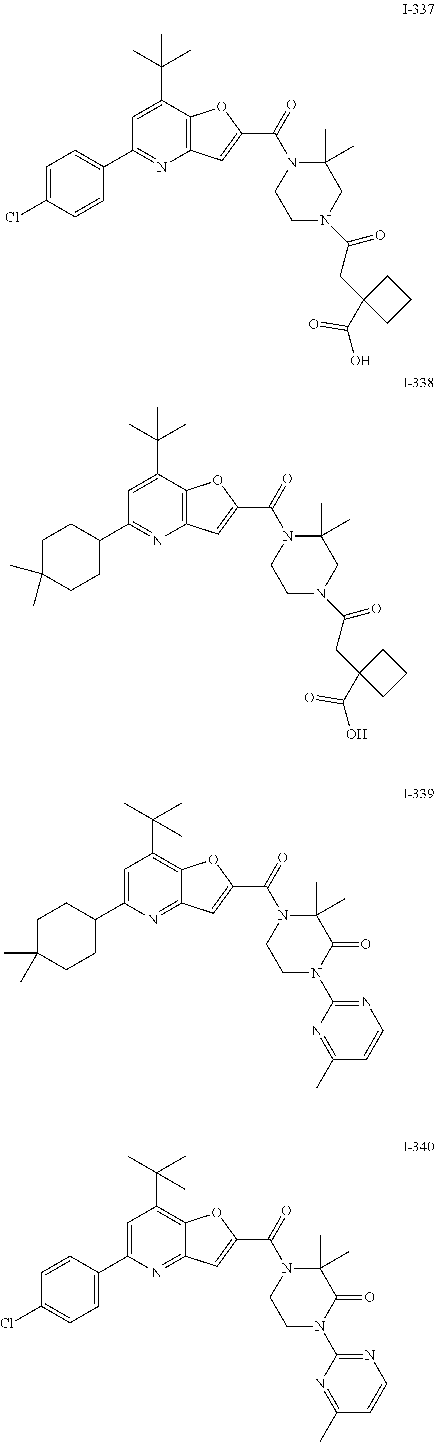 [SMILES: CC(C)(C)c1cc(-c2ccc(Cl)cc2)nc2cc(C(=O)N3CCN(C(=O)CC4(C(=O)O)CCC4)CC3(C)C)oc12 ; CC1(C)CCC(c2cc(C(C)(C)C)c3oc(C(=O)N4CCN(C(=O)CC5(C(=O)O)CCC5)CC4(C)C)cc3n2)CC1 ; Cc1ccnc(N2CCN(C(=O)c3cc4nc(C5CCC(C)(C)CC5)cc(C(C)(C)C)c4o3)C(C)(C)C2=O)n1 ; Cc1ccnc(N2CCN(C(=O)c3cc4nc(-c5ccc(Cl)cc5)cc(C(C)(C)C)c4o3)C(C)(C)C2=O)n1]

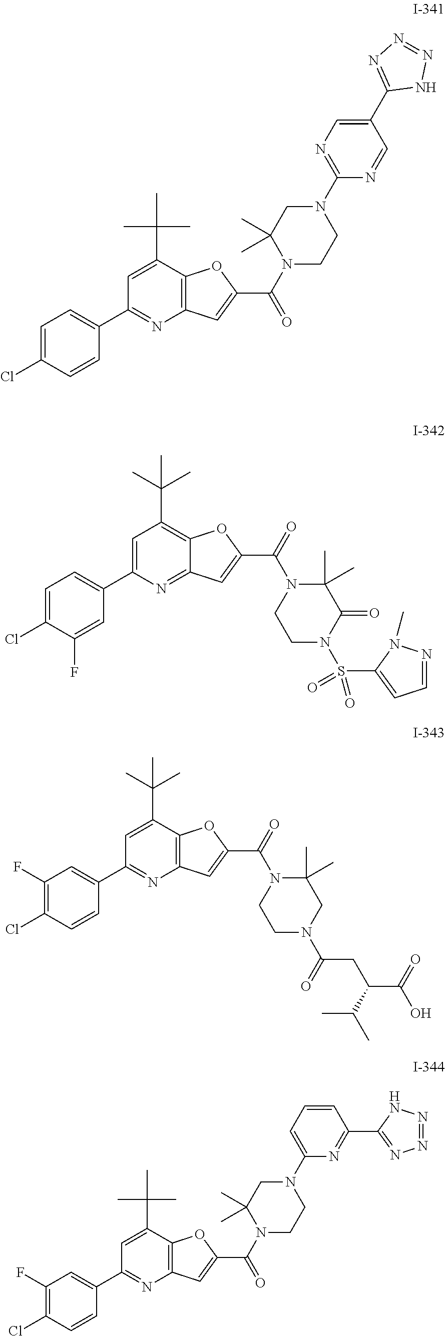 [SMILES: CC(C)(C)c1cc(-c2ccc(Cl)cc2)nc2cc(C(=O)N3CCN(c4ncc(-c5nnn[nH]5)cn4)CC3(C)C)oc12 ; Cn1nccc1S(=O)(=O)N1CCN(C(=O)c2cc3nc(-c4ccc(Cl)c(F)c4)cc(C(C)(C)C)c3o2)C(C)(C)C1=O ; CC(C)[C@H](CC(=O)N1CCN(C(=O)c2cc3nc(-c4ccc(Cl)c(F)c4)cc(C(C)(C)C)c3o2)C(C)(C)C1)C(=O)O ; CC(C)(C)c1cc(-c2ccc(Cl)c(F)c2)nc2cc(C(=O)N3CCN(c4cccc(-c5nnn[nH]5)n4)CC3(C)C)oc12]

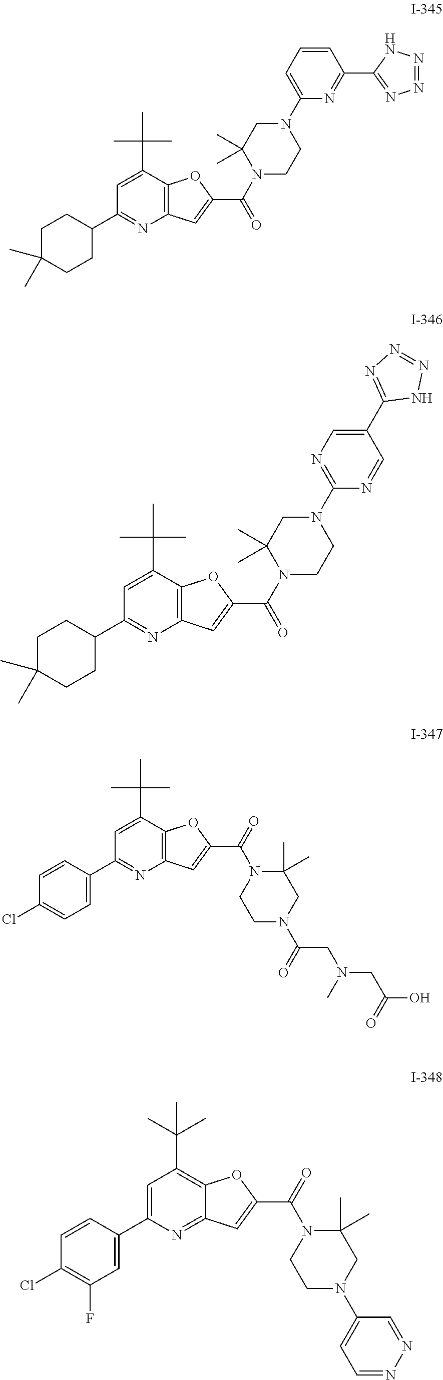 [SMILES: CC1(C)CCC(c2cc(C(C)(C)C)c3oc(C(=O)N4CCN(c5cccc(-c6nnn[nH]6)n5)CC4(C)C)cc3n2)CC1 ; CC1(C)CCC(c2cc(C(C)(C)C)c3oc(C(=O)N4CCN(c5ncc(-c6nnn[nH]6)cn5)CC4(C)C)cc3n2)CC1 ; CN(CC(=O)O)CC(=O)N1CCN(C(=O)c2cc3nc(-c4ccc(Cl)cc4)cc(C(C)(C)C)c3o2)C(C)(C)C1 ; CC(C)(C)c1cc(-c2ccc(Cl)c(F)c2)nc2cc(C(=O)N3CCN(c4ccnnc4)CC3(C)C)oc12]

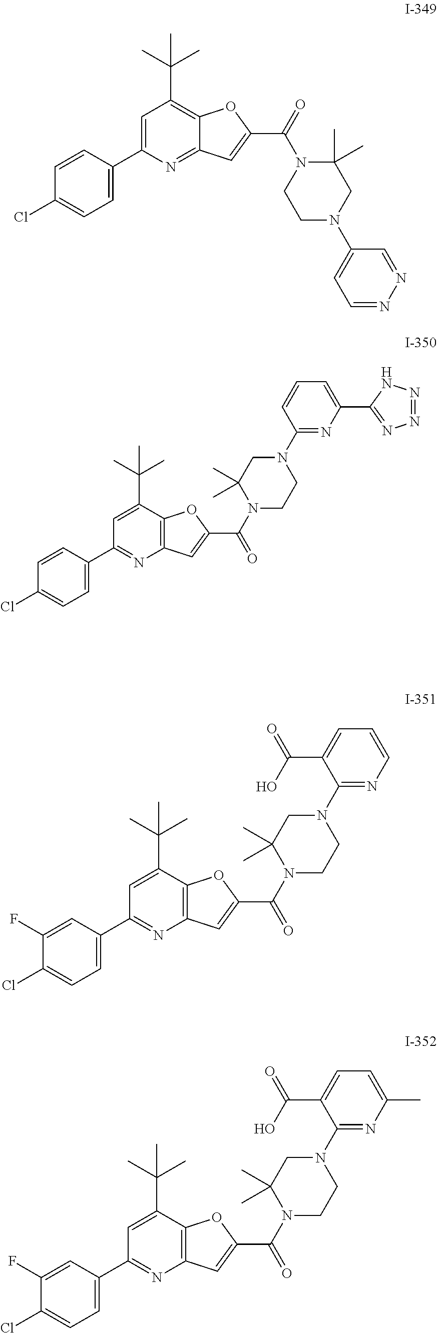 [SMILES: CC(C)(C)c1cc(-c2ccc(Cl)cc2)nc2cc(C(=O)N3CCN(c4ccnnc4)CC3(C)C)oc12 ; CC(C)(C)c1cc(-c2ccc(Cl)cc2)nc2cc(C(=O)N3CCN(c4cccc(-c5nnn[nH]5)n4)CC3(C)C)oc12 ; CC(C)(C)c1cc(-c2ccc(Cl)c(F)c2)nc2cc(C(=O)N3CCN(c4ncccc4C(=O)O)CC3(C)C)oc12 ; Cc1ccc(C(=O)O)c(N2CCN(C(=O)c3cc4nc(-c5ccc(Cl)c(F)c5)cc(C(C)(C)C)c4o3)C(C)(C)C2)n1]

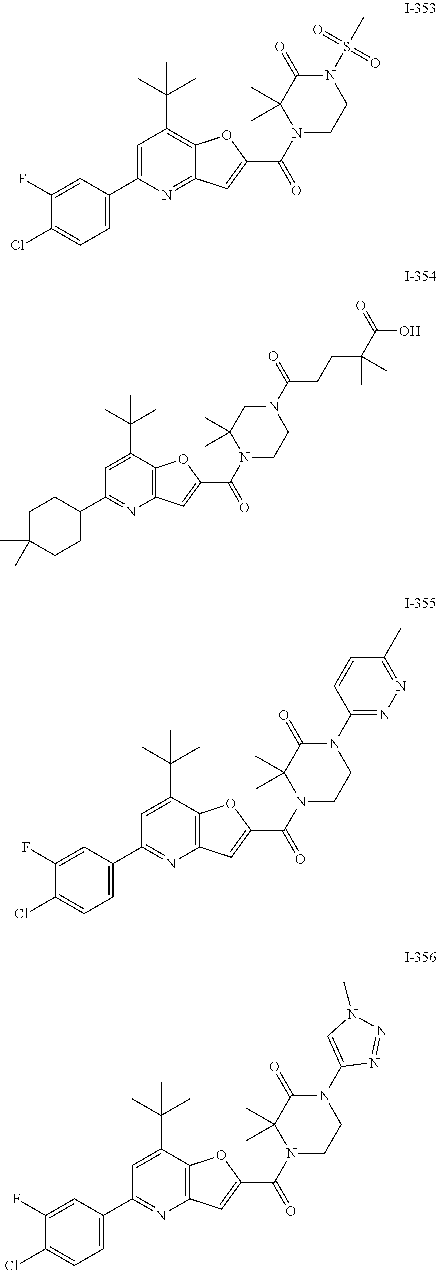 [SMILES: CC(C)(C)c1cc(-c2ccc(Cl)c(F)c2)nc2cc(C(=O)N3CCN(S(C)(=O)=O)C(=O)C3(C)C)oc12 ; CC1(C)CCC(c2cc(C(C)(C)C)c3oc(C(=O)N4CCN(C(=O)CCC(C)(C)C(=O)O)CC4(C)C)cc3n2)CC1 ; Cc1ccc(N2CCN(C(=O)c3cc4nc(-c5ccc(Cl)c(F)c5)cc(C(C)(C)C)c4o3)C(C)(C)C2=O)nn1 ; Cn1cc(N2CCN(C(=O)c3cc4nc(-c5ccc(Cl)c(F)c5)cc(C(C)(C)C)c4o3)C(C)(C)C2=O)nn1]

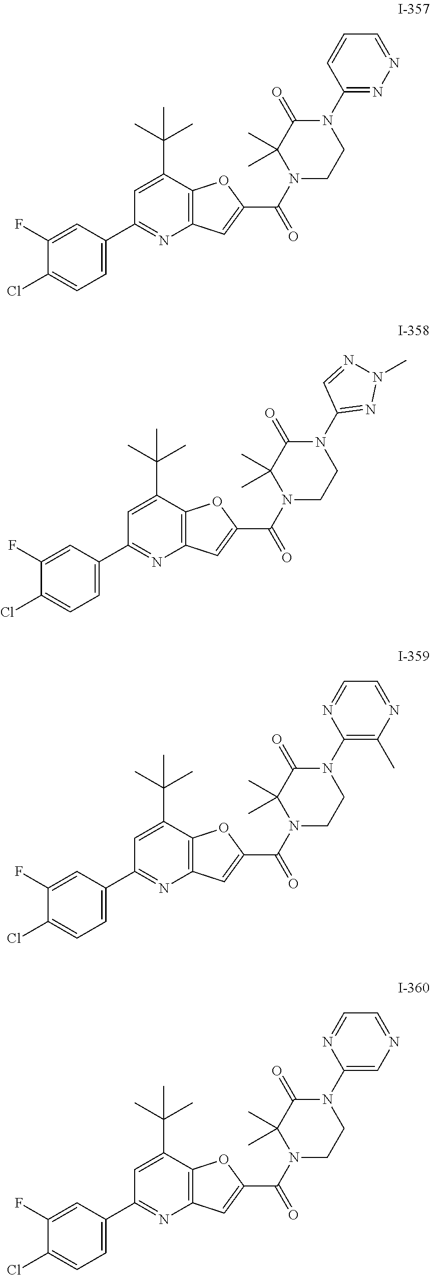 [SMILES: CC(C)(C)c1cc(-c2ccc(Cl)c(F)c2)nc2cc(C(=O)N3CCN(c4cccnn4)C(=O)C3(C)C)oc12 ; Cn1ncc(N2CCN(C(=O)c3cc4nc(-c5ccc(Cl)c(F)c5)cc(C(C)(C)C)c4o3)C(C)(C)C2=O)n1 ; Cc1nccnc1N1CCN(C(=O)c2cc3nc(-c4ccc(Cl)c(F)c4)cc(C(C)(C)C)c3o2)C(C)(C)C1=O ; CC(C)(C)c1cc(-c2ccc(Cl)c(F)c2)nc2cc(C(=O)N3CCN(c4cnccn4)C(=O)C3(C)C)oc12]

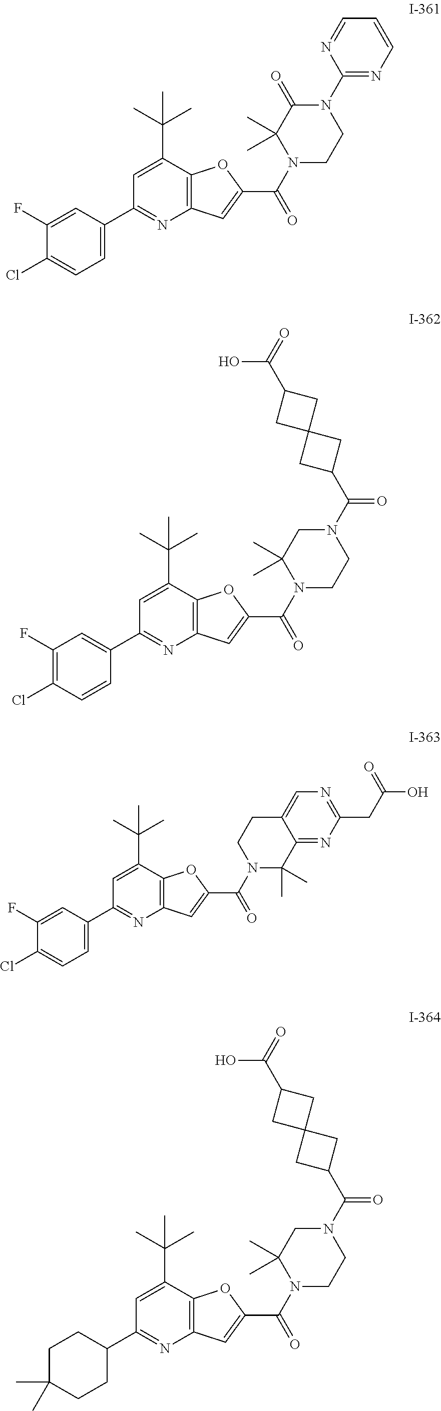 [SMILES: CC(C)(C)c1cc(-c2ccc(Cl)c(F)c2)nc2cc(C(=O)N3CCN(c4ncccn4)C(=O)C3(C)C)oc12 ; CC(C)(C)c1cc(-c2ccc(Cl)c(F)c2)nc2cc(C(=O)N3CCN(C(=O)C4CC5(CC(C(=O)O)C5)C4)CC3(C)C)oc12 ; CC(C)(C)c1cc(-c2ccc(Cl)c(F)c2)nc2cc(C(=O)N3CCc4cnc(CC(=O)O)nc4C3(C)C)oc12 ; CC1(C)CCC(c2cc(C(C)(C)C)c3oc(C(=O)N4CCN(C(=O)C5CC6(CC(C(=O)O)C6)C5)CC4(C)C)cc3n2)CC1]

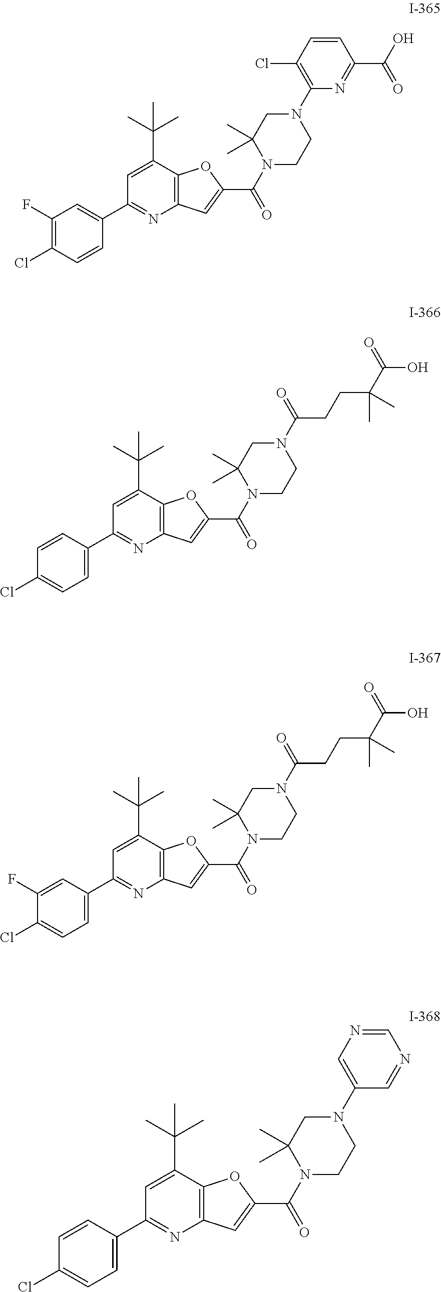 [SMILES: CC(C)(C)c1cc(-c2ccc(Cl)c(F)c2)nc2cc(C(=O)N3CCN(c4nc(C(=O)O)ccc4Cl)CC3(C)C)oc12 ; CC(C)(CCC(=O)N1CCN(C(=O)c2cc3nc(-c4ccc(Cl)cc4)cc(C(C)(C)C)c3o2)C(C)(C)C1)C(=O)O ; CC(C)(CCC(=O)N1CCN(C(=O)c2cc3nc(-c4ccc(Cl)c(F)c4)cc(C(C)(C)C)c3o2)C(C)(C)C1)C(=O)O ; CC(C)(C)c1cc(-c2ccc(Cl)cc2)nc2cc(C(=O)N3CCN(c4cncnc4)CC3(C)C)oc12]

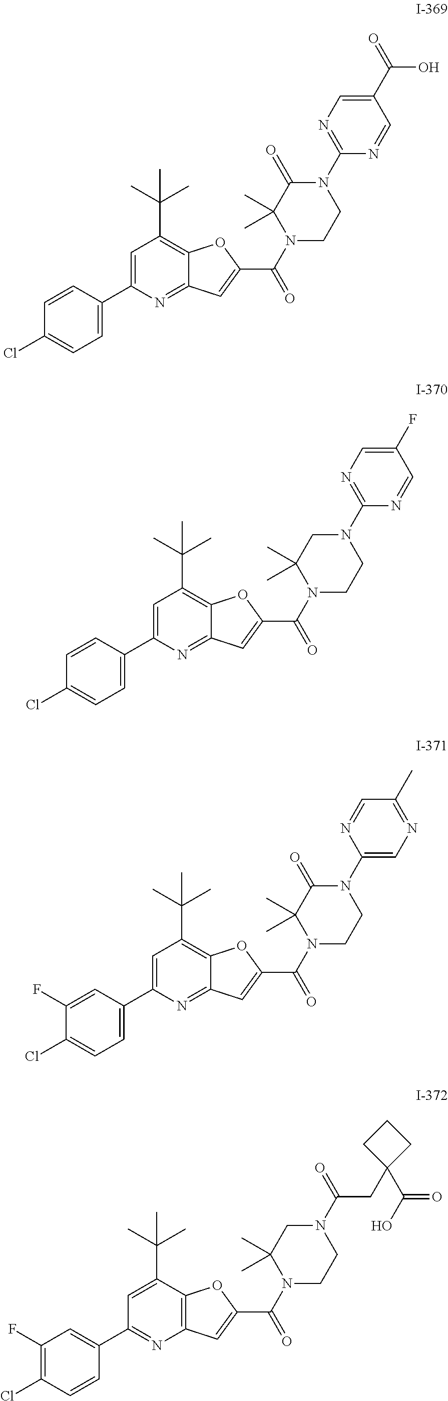 [SMILES: CC(C)(C)c1cc(-c2ccc(Cl)cc2)nc2cc(C(=O)N3CCN(c4ncc(C(=O)O)cn4)C(=O)C3(C)C)oc12 ; CC(C)(C)c1cc(-c2ccc(Cl)cc2)nc2cc(C(=O)N3CCN(c4ncc(F)cn4)CC3(C)C)oc12 ; Cc1cnc(N2CCN(C(=O)c3cc4nc(-c5ccc(Cl)c(F)c5)cc(C(C)(C)C)c4o3)C(C)(C)C2=O)cn1 ; CC(C)(C)c1cc(-c2ccc(Cl)c(F)c2)nc2cc(C(=O)N3CCN(C(=O)CC4(C(=O)O)CCC4)CC3(C)C)oc12]

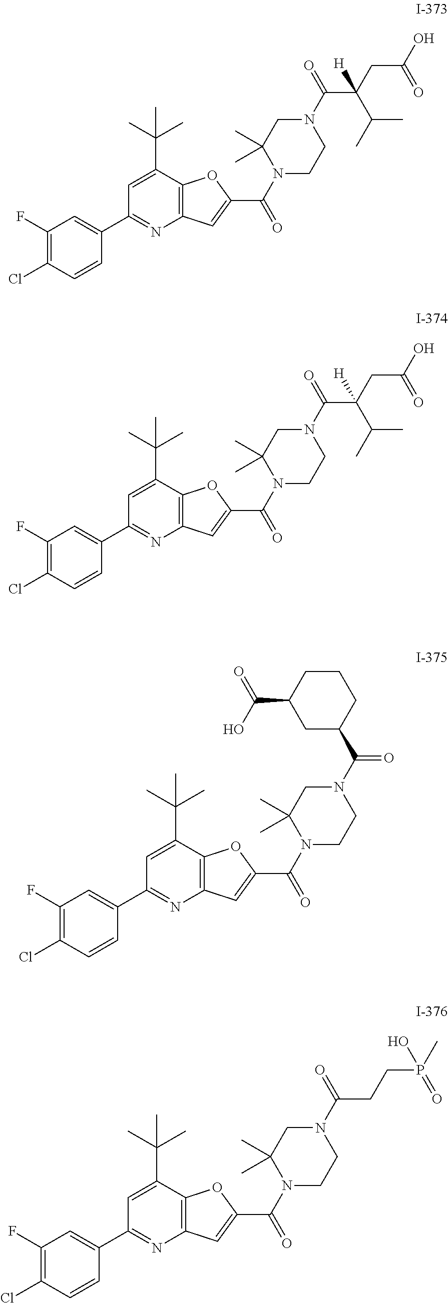 [SMILES: CC(C)[C@@H](CC(=O)O)C(=O)N1CCN(C(=O)c2cc3nc(-c4ccc(Cl)c(F)c4)cc(C(C)(C)C)c3o2)C(C)(C)C1 ; CC(C)[C@H](CC(=O)O)C(=O)N1CCN(C(=O)c2cc3nc(-c4ccc(Cl)c(F)c4)cc(C(C)(C)C)c3o2)C(C)(C)C1 ; CC(C)(C)c1cc(-c2ccc(Cl)c(F)c2)nc2cc(C(=O)N3CCN(C(=O)[C@@H]4CCC[C@H](C(=O)O)C4)CC3(C)C)oc12 ; CC(C)(C)c1cc(-c2ccc(Cl)c(F)c2)nc2cc(C(=O)N3CCN(C(=O)CCP(C)(=O)O)CC3(C)C)oc12]

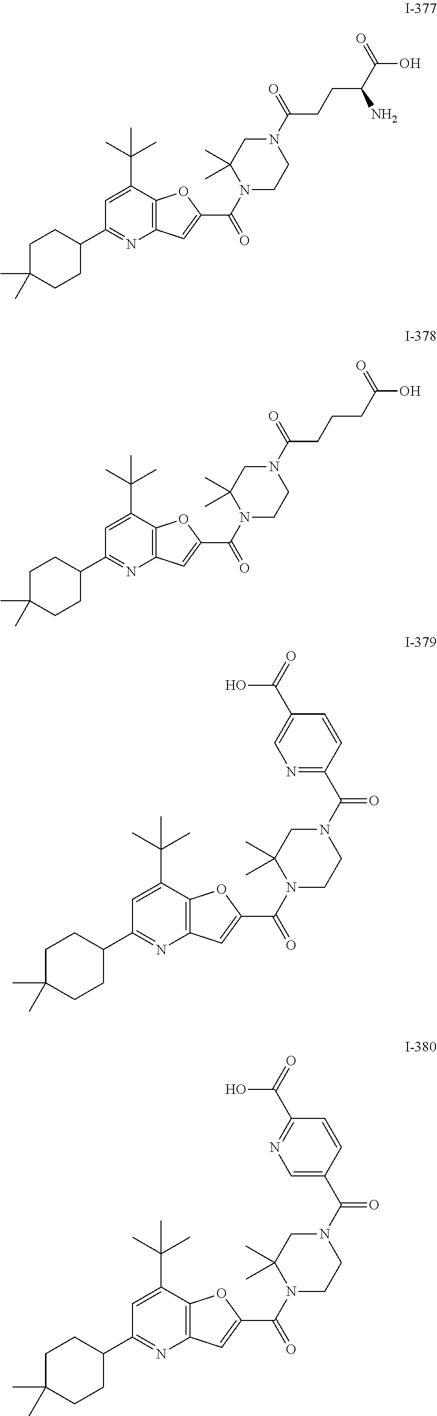 [SMILES: CC1(C)CCC(c2cc(C(C)(C)C)c3oc(C(=O)N4CCN(C(=O)CC[C@H](N)C(=O)O)CC4(C)C)cc3n2)CC1 ; CC1(C)CCC(c2cc(C(C)(C)C)c3oc(C(=O)N4CCN(C(=O)CCCC(=O)O)CC4(C)C)cc3n2)CC1 ; CC1(C)CCC(c2cc(C(C)(C)C)c3oc(C(=O)N4CCN(C(=O)c5ccc(C(=O)O)cn5)CC4(C)C)cc3n2)CC1 ; CC1(C)CCC(c2cc(C(C)(C)C)c3oc(C(=O)N4CCN(C(=O)c5ccc(C(=O)O)nc5)CC4(C)C)cc3n2)CC1]

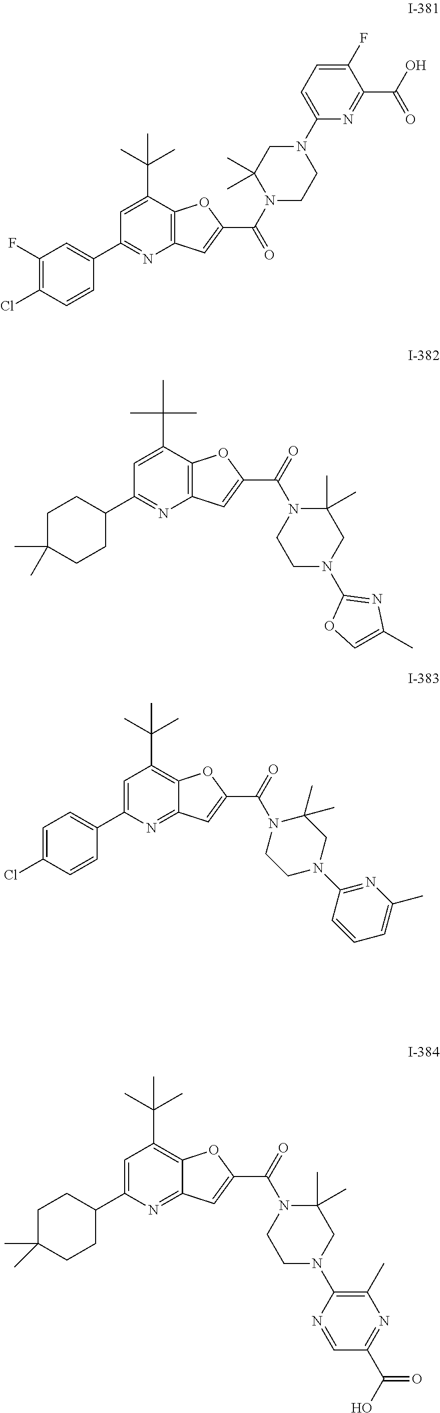

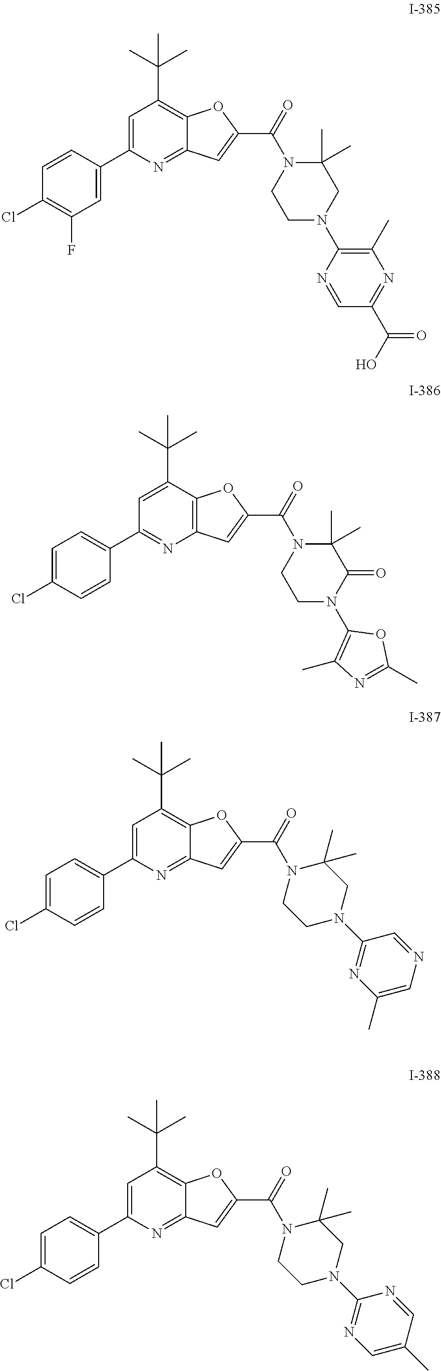 [SMILES: Cc1nc(C(=O)O)cnc1N1CCN(C(=O)c2cc3nc(-c4ccc(Cl)c(F)c4)cc(C(C)(C)C)c3o2)C(C)(C)C1 ; Cc1nc(C)c(N2CCN(C(=O)c3cc4nc(-c5ccc(Cl)cc5)cc(C(C)(C)C)c4o3)C(C)(C)C2=O)o1 ; Cc1cncc(N2CCN(C(=O)c3cc4nc(-c5ccc(Cl)cc5)cc(C(C)(C)C)c4o3)C(C)(C)C2)n1 ; Cc1cnc(N2CCN(C(=O)c3cc4nc(-c5ccc(Cl)cc5)cc(C(C)(C)C)c4o3)C(C)(C)C2)nc1]

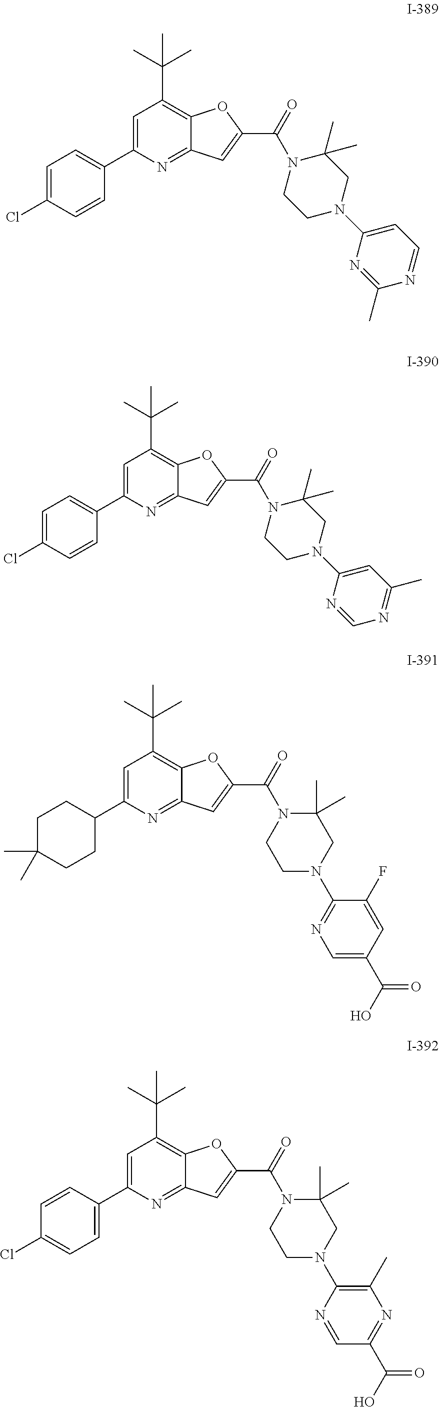

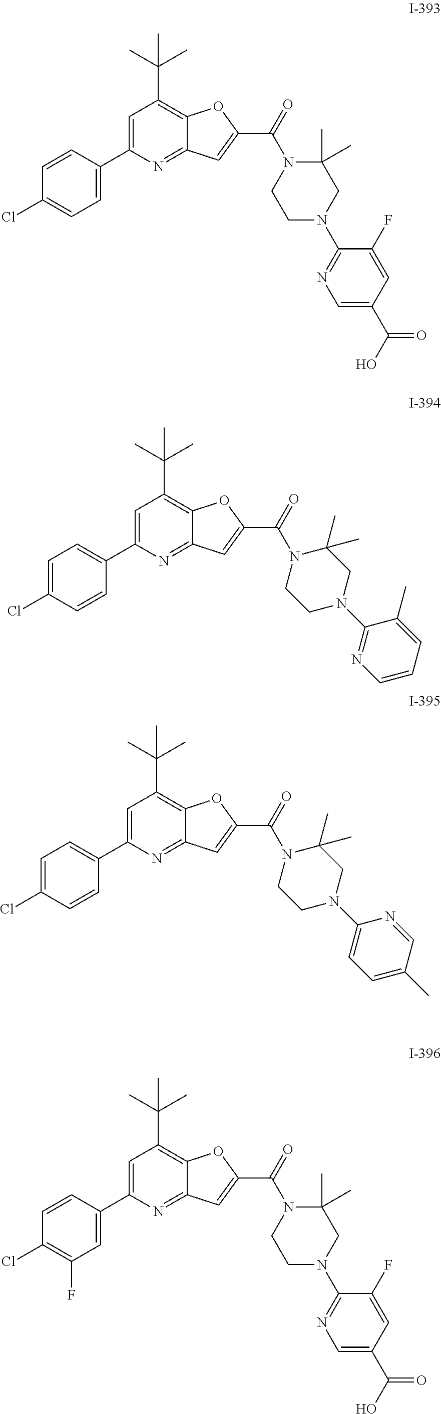 [SMILES: CC(C)(C)c1cc(-c2ccc(Cl)cc2)nc2cc(C(=O)N3CCN(c4ncc(C(=O)O)cc4F)CC3(C)C)oc12 ; Cc1cccnc1N1CCN(C(=O)c2cc3nc(-c4ccc(Cl)cc4)cc(C(C)(C)C)c3o2)C(C)(C)C1 ; Cc1ccc(N2CCN(C(=O)c3cc4nc(-c5ccc(Cl)cc5)cc(C(C)(C)C)c4o3)C(C)(C)C2)nc1 ; CC(C)(C)c1cc(-c2ccc(Cl)c(F)c2)nc2cc(C(=O)N3CCN(c4ncc(C(=O)O)cc4F)CC3(C)C)oc12]

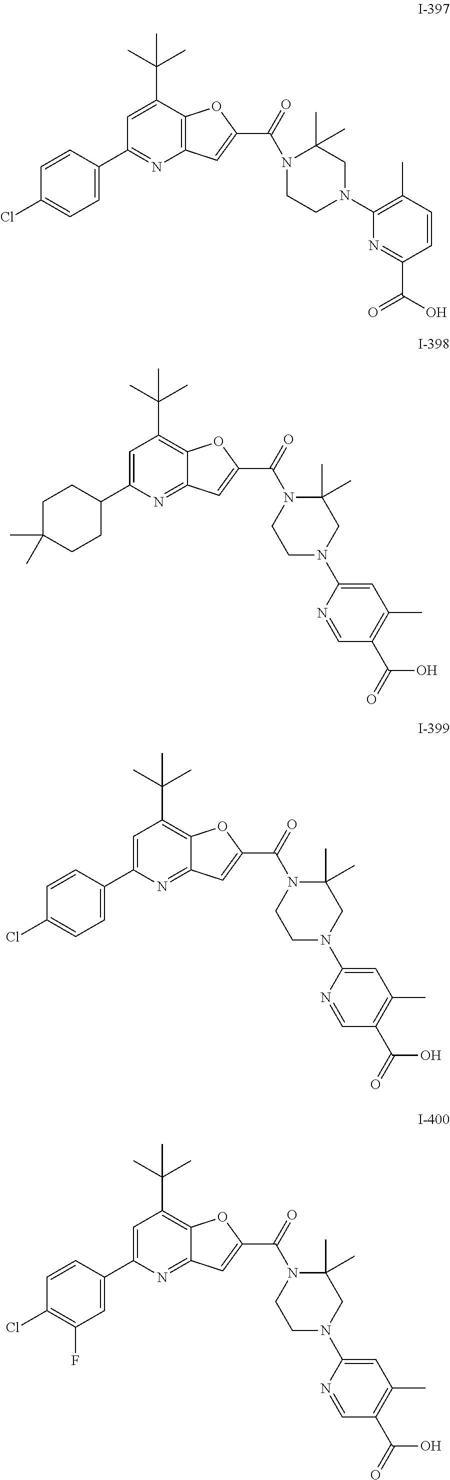

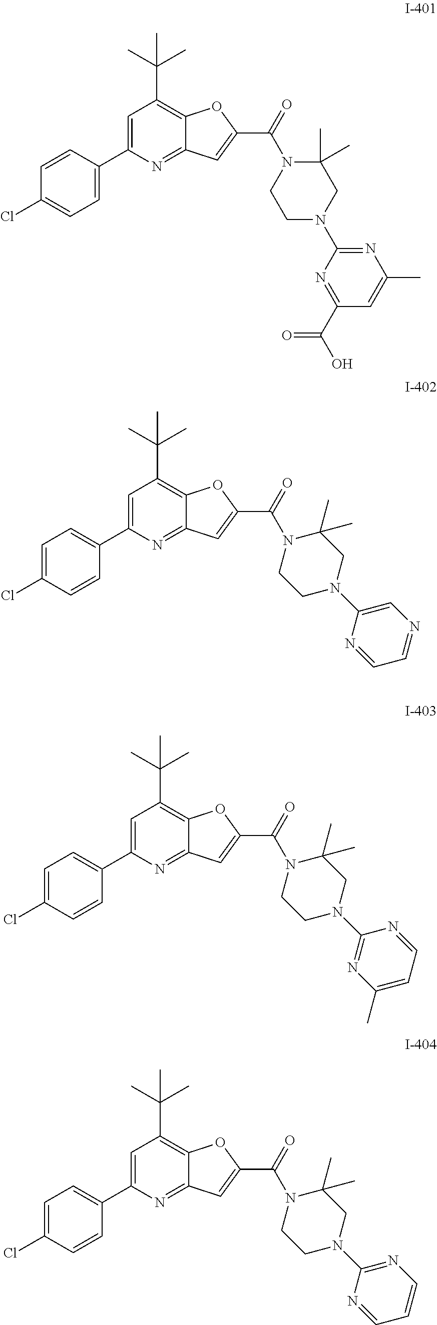 [SMILES: Cc1cc(C(=O)O)nc(N2CCN(C(=O)c3cc4nc(-c5ccc(Cl)cc5)cc(C(C)(C)C)c4o3)C(C)(C)C2)n1 ; CC(C)(C)c1cc(-c2ccc(Cl)cc2)nc2cc(C(=O)N3CCN(c4cnccn4)CC3(C)C)oc12 ; Cc1ccnc(N2CCN(C(=O)c3cc4nc(-c5ccc(Cl)cc5)cc(C(C)(C)C)c4o3)C(C)(C)C2)n1 ; CC(C)(C)c1cc(-c2ccc(Cl)cc2)nc2cc(C(=O)N3CCN(c4ncccn4)CC3(C)C)oc12]

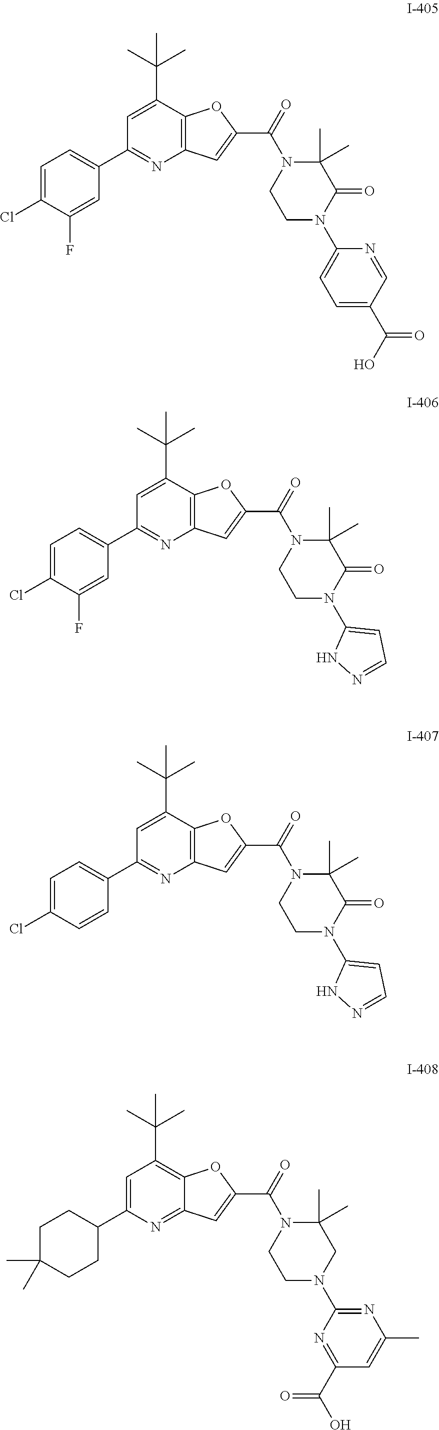

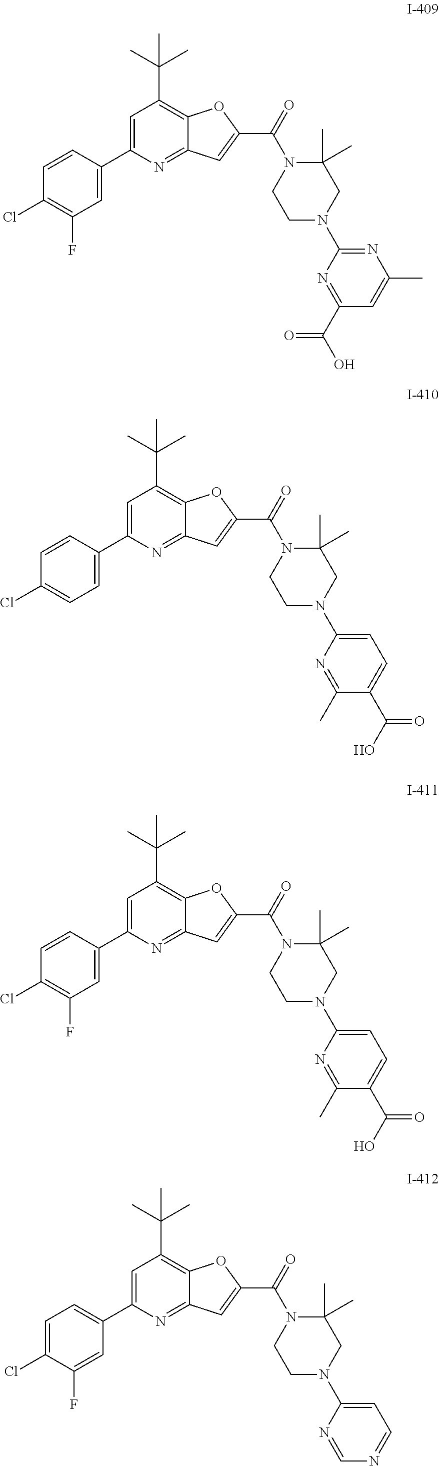 [SMILES: Cc1cc(C(=O)O)nc(N2CCN(C(=O)c3cc4nc(-c5ccc(Cl)c(F)c5)cc(C(C)(C)C)c4o3)C(C)(C)C2)n1 ; Cc1nc(N2CCN(C(=O)c3cc4nc(-c5ccc(Cl)cc5)cc(C(C)(C)C)c4o3)C(C)(C)C2)ccc1C(=O)O ; Cc1nc(N2CCN(C(=O)c3cc4nc(-c5ccc(Cl)c(F)c5)cc(C(C)(C)C)c4o3)C(C)(C)C2)ccc1C(=O)O ; CC(C)(C)c1cc(-c2ccc(Cl)c(F)c2)nc2cc(C(=O)N3CCN(c4ccncn4)CC3(C)C)oc12]

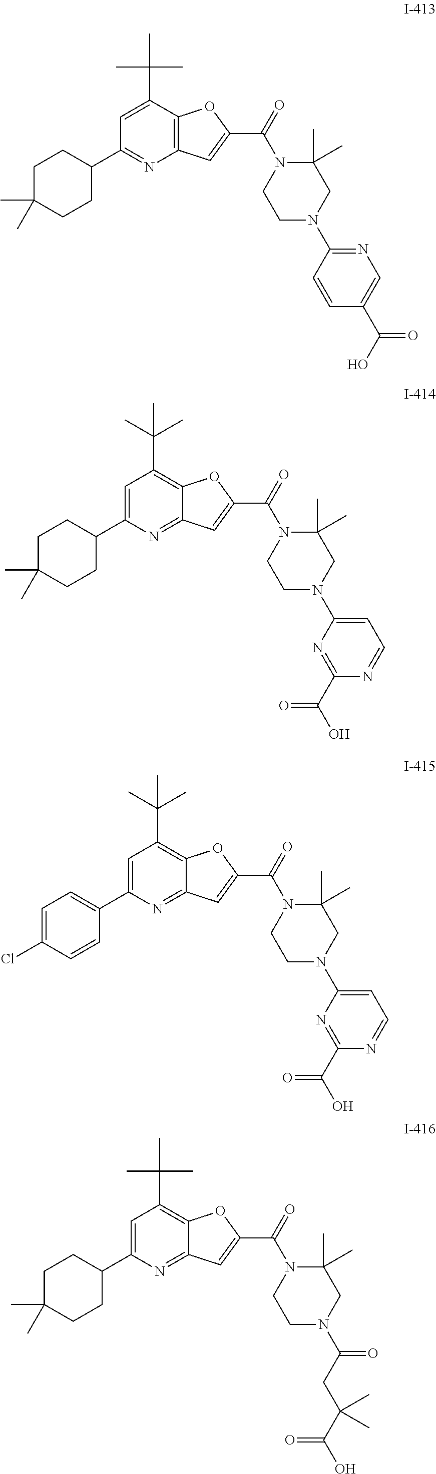 [SMILES: CC1(C)CCC(c2cc(C(C)(C)C)c3oc(C(=O)N4CCN(c5ccc(C(=O)O)cn5)CC4(C)C)cc3n2)CC1 ; CC1(C)CCC(c2cc(C(C)(C)C)c3oc(C(=O)N4CCN(c5ccnc(C(=O)O)n5)CC4(C)C)cc3n2)CC1 ; CC(C)(C)c1cc(-c2ccc(Cl)cc2)nc2cc(C(=O)N3CCN(c4ccnc(C(=O)O)n4)CC3(C)C)oc12 ; CC1(C)CCC(c2cc(C(C)(C)C)c3oc(C(=O)N4CCN(C(=O)CC(C)(C)C(=O)O)CC4(C)C)cc3n2)CC1]

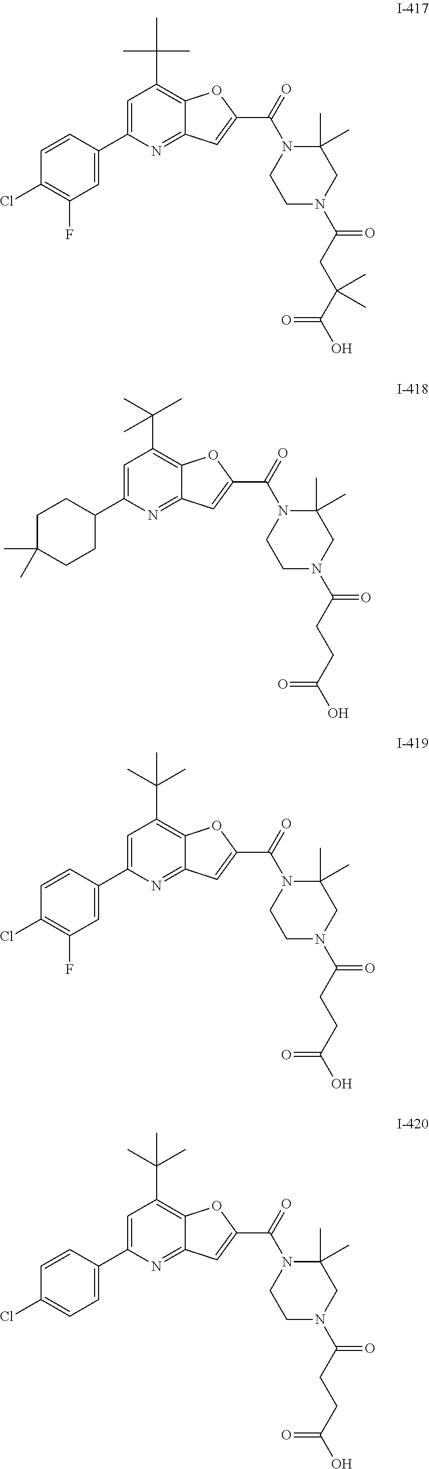 [SMILES: CC(C)(CC(=O)N1CCN(C(=O)c2cc3nc(-c4ccc(Cl)c(F)c4)cc(C(C)(C)C)c3o2)C(C)(C)C1)C(=O)O ; CC1(C)CCC(c2cc(C(C)(C)C)c3oc(C(=O)N4CCN(C(=O)CCC(=O)O)CC4(C)C)cc3n2)CC1 ; CC(C)(C)c1cc(-c2ccc(Cl)c(F)c2)nc2cc(C(=O)N3CCN(C(=O)CCC(=O)O)CC3(C)C)oc12 ; CC(C)(C)c1cc(-c2ccc(Cl)cc2)nc2cc(C(=O)N3CCN(C(=O)CCC(=O)O)CC3(C)C)oc12]

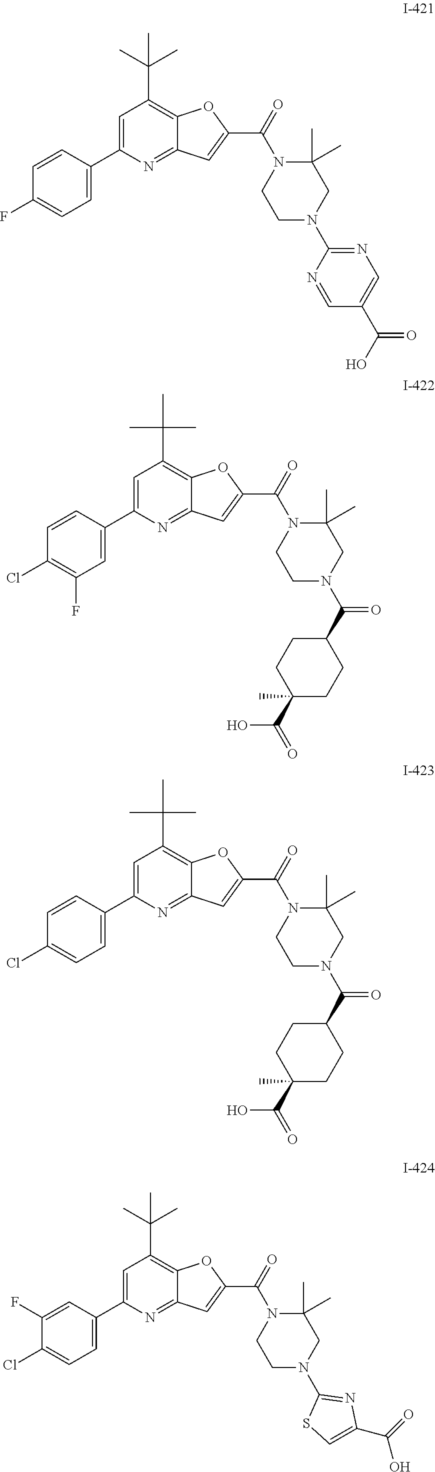 [SMILES: CC(C)(C)c1cc(-c2ccc(F)cc2)nc2cc(C(=O)N3CCN(c4ncc(C(=O)O)cn4)CC3(C)C)oc12 ; CC(C)(C)c1cc(-c2ccc(Cl)c(F)c2)nc2cc(C(=O)N3CCN(C(=O)[C@H]4CC[C@@](C)(C(=O)O)CC4)CC3(C)C)oc12 ; CC(C)(C)c1cc(-c2ccc(Cl)cc2)nc2cc(C(=O)N3CCN(C(=O)[C@H]4CC[C@@](C)(C(=O)O)CC4)CC3(C)C)oc12 ; CC(C)(C)c1cc(-c2ccc(Cl)c(F)c2)nc2cc(C(=O)N3CCN(c4nc(C(=O)O)cs4)CC3(C)C)oc12]

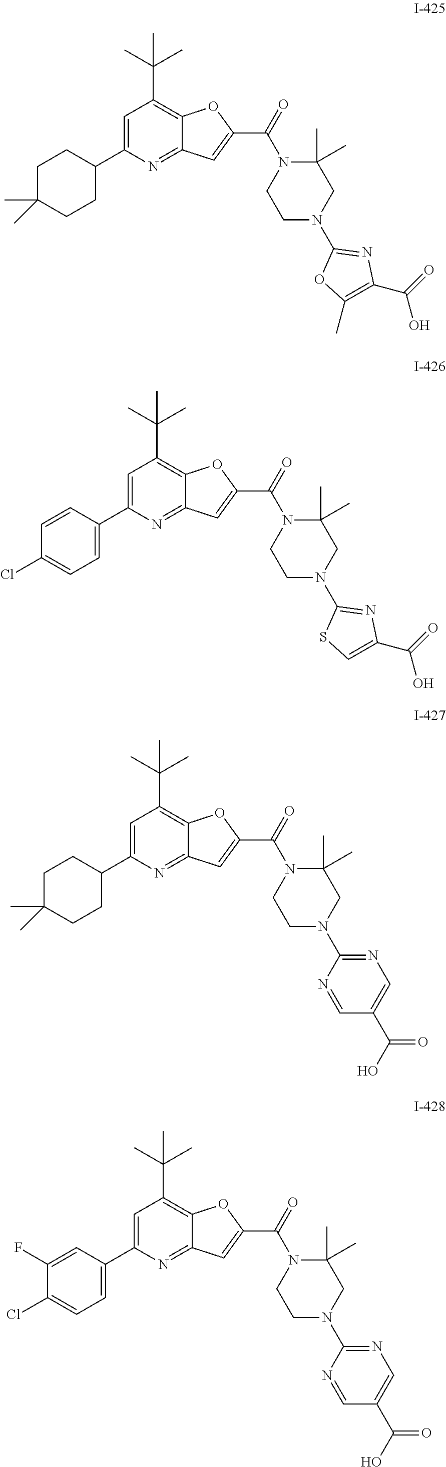 [SMILES: Cc1oc(N2CCN(C(=O)c3cc4nc(C5CCC(C)(C)CC5)cc(C(C)(C)C)c4o3)C(C)(C)C2)nc1C(=O)O ; CC(C)(C)c1cc(-c2ccc(Cl)cc2)nc2cc(C(=O)N3CCN(c4nc(C(=O)O)cs4)CC3(C)C)oc12 ; CC1(C)CCC(c2cc(C(C)(C)C)c3oc(C(=O)N4CCN(c5ncc(C(=O)O)cn5)CC4(C)C)cc3n2)CC1 ; CC(C)(C)c1cc(-c2ccc(Cl)c(F)c2)nc2cc(C(=O)N3CCN(c4ncc(C(=O)O)cn4)CC3(C)C)oc12]

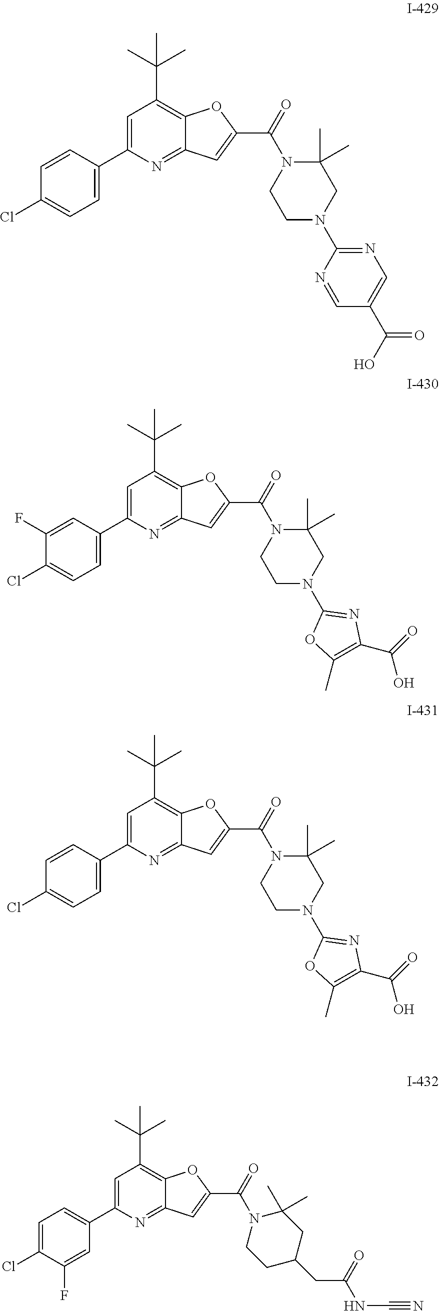 [SMILES: CC(C)(C)c1cc(-c2ccc(Cl)cc2)nc2cc(C(=O)N3CCN(c4ncc(C(=O)O)cn4)CC3(C)C)oc12 ; Cc1oc(N2CCN(C(=O)c3cc4nc(-c5ccc(Cl)c(F)c5)cc(C(C)(C)C)c4o3)C(C)(C)C2)nc1C(=O)O ; Cc1oc(N2CCN(C(=O)c3cc4nc(-c5ccc(Cl)cc5)cc(C(C)(C)C)c4o3)C(C)(C)C2)nc1C(=O)O ; CC(C)(C)c1cc(-c2ccc(Cl)c(F)c2)nc2cc(C(=O)N3CCC(CC(=O)NC#N)CC3(C)C)oc12]

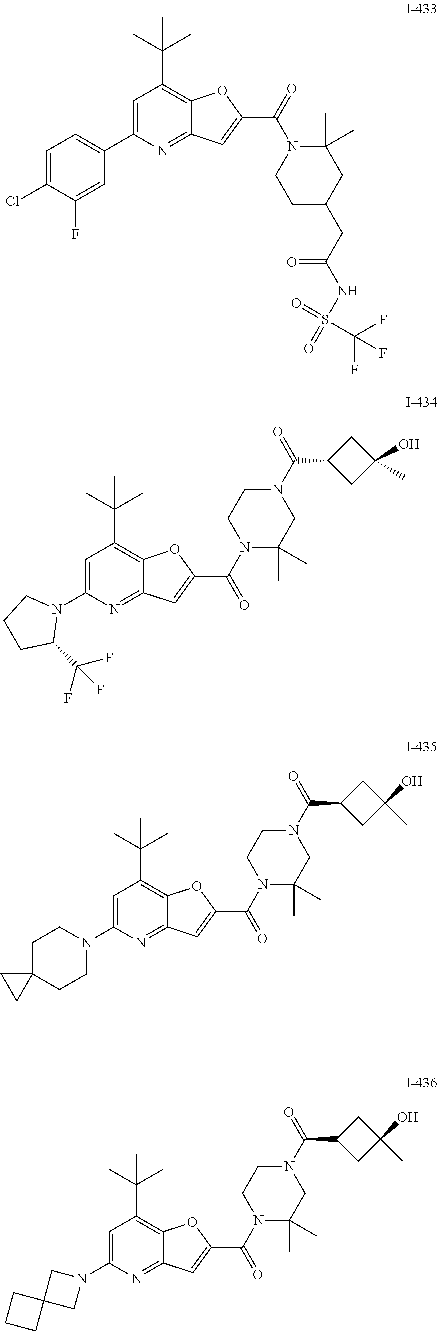

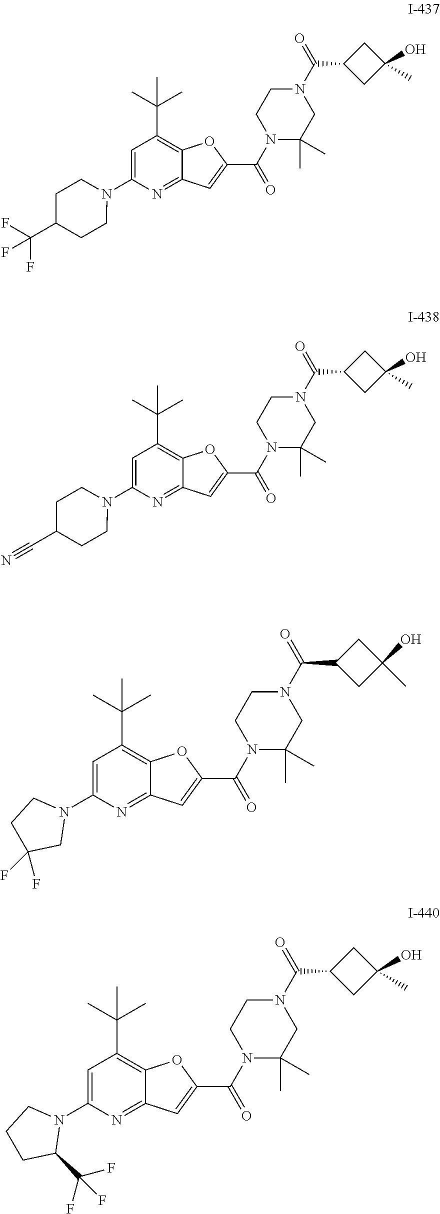

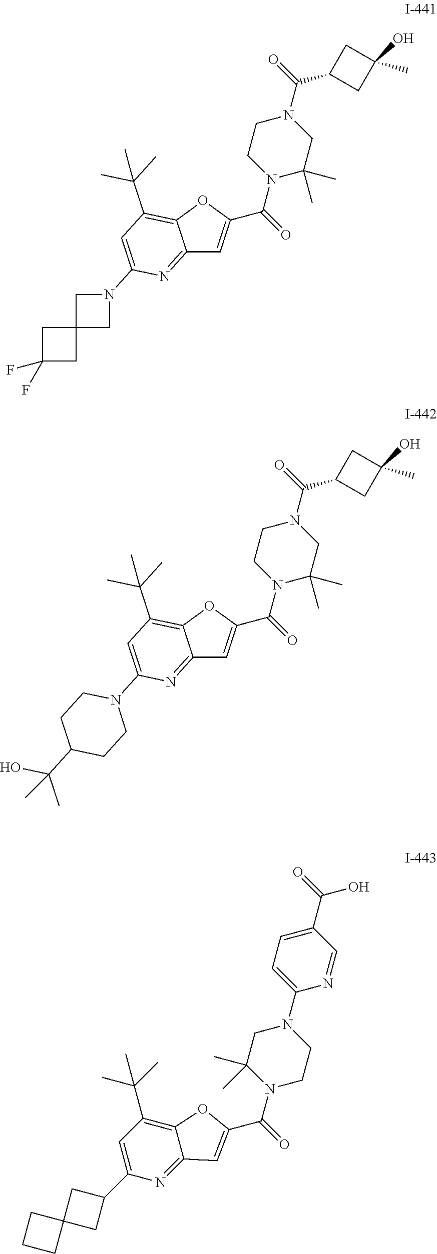 [SMILES: CC(C)(C)c1cc(N2CC3(C2)CC(F)(F)C3)nc2cc(C(=O)N3CCN(C(=O)[C@H]4C[C@](C)(O)C4)CC3(C)C)oc12 ; CC(C)(C)c1cc(N2CCC(C(C)(C)O)CC2)nc2cc(C(=O)N3CCN(C(=O)[C@H]4C[C@](C)(O)C4)CC3(C)C)oc12 ; CC(C)(C)c1cc(C2CC3(CCC3)C2)nc2cc(C(=O)N3CCN(c4ccc(C(=O)O)cn4)CC3(C)C)oc12]

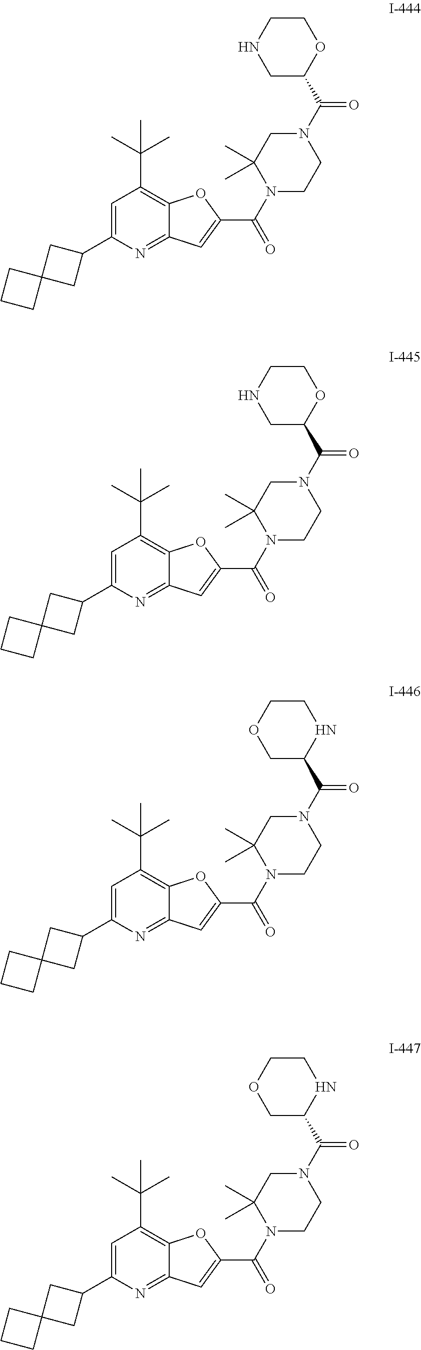 [SMILES: CC(C)(C)c1cc(C2CC3(CCC3)C2)nc2cc(C(=O)N3CCN(C(=O)[C@@H]4CNCCO4)CC3(C)C)oc12 ; CC(C)(C)c1cc(C2CC3(CCC3)C2)nc2cc(C(=O)N3CCN(C(=O)[C@H]4CNCCO4)CC3(C)C)oc12 ; CC(C)(C)c1cc(C2CC3(CCC3)C2)nc2cc(C(=O)N3CCN(C(=O)[C@H]4COCCN4)CC3(C)C)oc12 ; CC(C)(C)c1cc(C2CC3(CCC3)C2)nc2cc(C(=O)N3CCN(C(=O)[C@@H]4COCCN4)CC3(C)C)oc12]

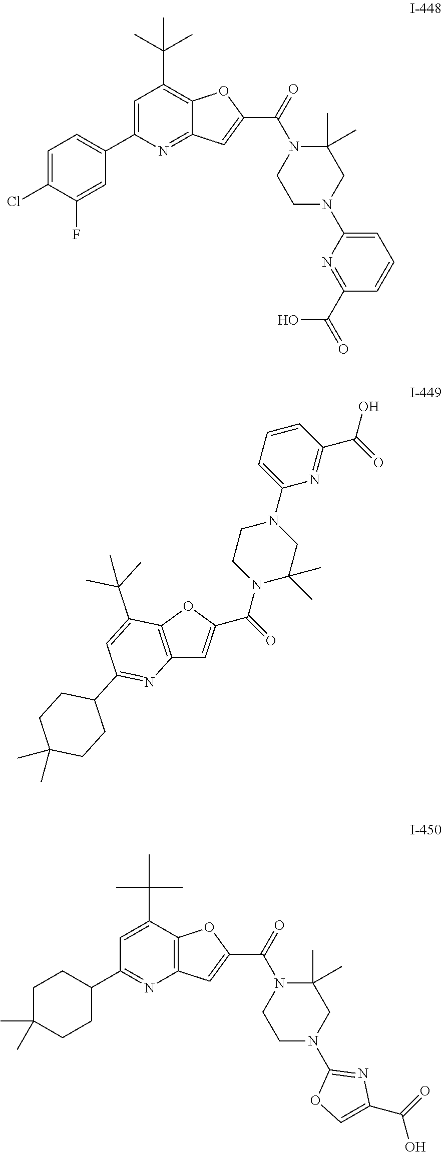 [SMILES: CC(C)(C)c1cc(-c2ccc(Cl)c(F)c2)nc2cc(C(=O)N3CCN(c4cccc(C(=O)O)n4)CC3(C)C)oc12 ; CC1(C)CCC(c2cc(C(C)(C)C)c3oc(C(=O)N4CCN(c5cccc(C(=O)O)n5)CC4(C)C)cc3n2)CC1 ; CC1(C)CCC(c2cc(C(C)(C)C)c3oc(C(=O)N4CCN(c5nc(C(=O)O)co5)CC4(C)C)cc3n2)CC1]

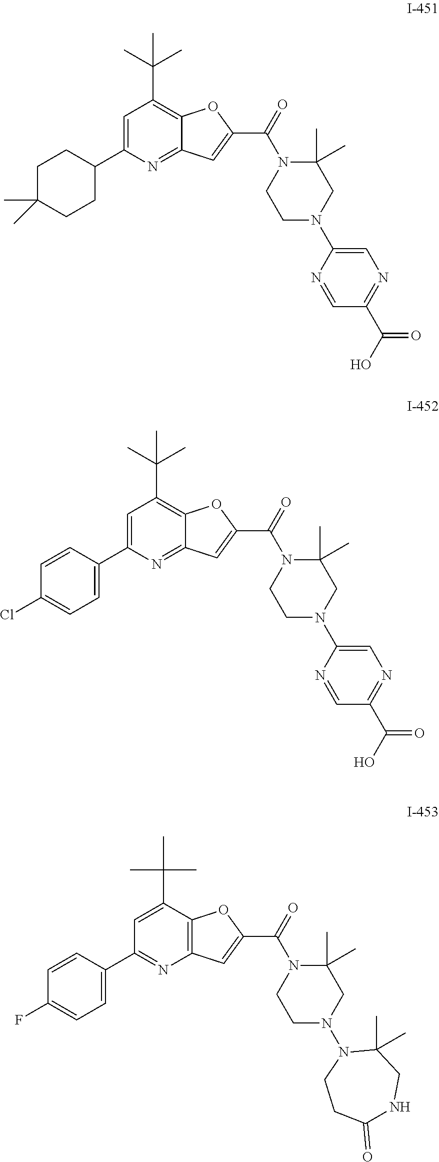 [SMILES: CC1(C)CCC(c2cc(C(C)(C)C)c3oc(C(=O)N4CCN(c5cnc(C(=O)O)cn5)CC4(C)C)cc3n2)CC1 ; CC(C)(C)c1cc(-c2ccc(Cl)cc2)nc2cc(C(=O)N3CCN(c4cnc(C(=O)O)cn4)CC3(C)C)oc12 ; CC(C)(C)c1cc(-c2ccc(F)cc2)nc2cc(C(=O)N3CCN(N4CCC(=O)NCC4(C)C)CC3(C)C)oc12]

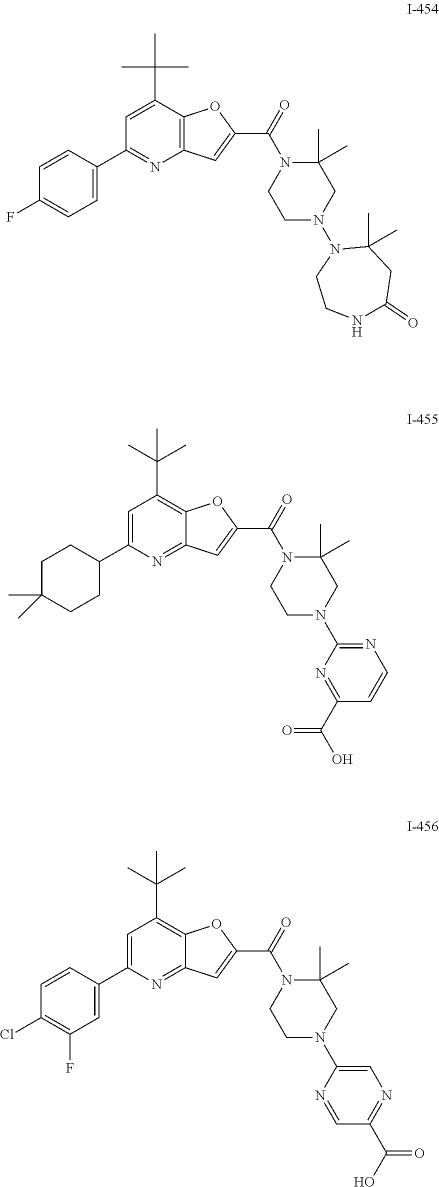 [SMILES: CC(C)(C)c1cc(-c2ccc(F)cc2)nc2cc(C(=O)N3CCN(N4CCNC(=O)CC4(C)C)CC3(C)C)oc12 ; CC1(C)CCC(c2cc(C(C)(C)C)c3oc(C(=O)N4CCN(c5nccc(C(=O)O)n5)CC4(C)C)cc3n2)CC1 ; CC(C)(C)c1cc(-c2ccc(Cl)c(F)c2)nc2cc(C(=O)N3CCN(c4cnc(C(=O)O)cn4)CC3(C)C)oc12]

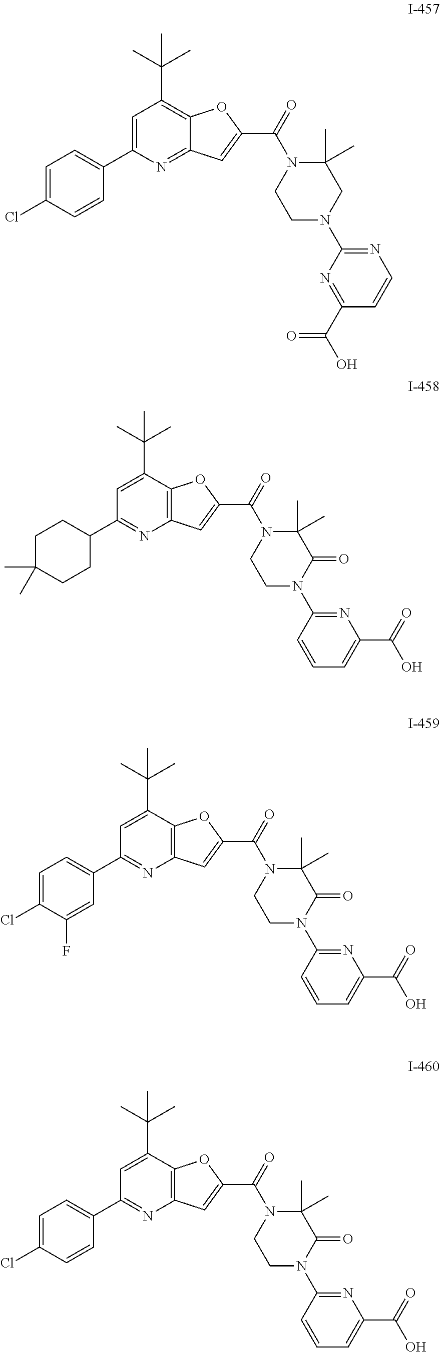 [SMILES: CC(C)(C)c1cc(-c2ccc(Cl)cc2)nc2cc(C(=O)N3CCN(c4nccc(C(=O)O)n4)CC3(C)C)oc12 ; CC1(C)CCC(c2cc(C(C)(C)C)c3oc(C(=O)N4CCN(c5cccc(C(=O)O)n5)C(=O)C4(C)C)cc3n2)CC1 ; CC(C)(C)c1cc(-c2ccc(Cl)c(F)c2)nc2cc(C(=O)N3CCN(c4cccc(C(=O)O)n4)C(=O)C3(C)C)oc12 ; CC(C)(C)c1cc(-c2ccc(Cl)cc2)nc2cc(C(=O)N3CCN(c4cccc(C(=O)O)n4)C(=O)C3(C)C)oc12]

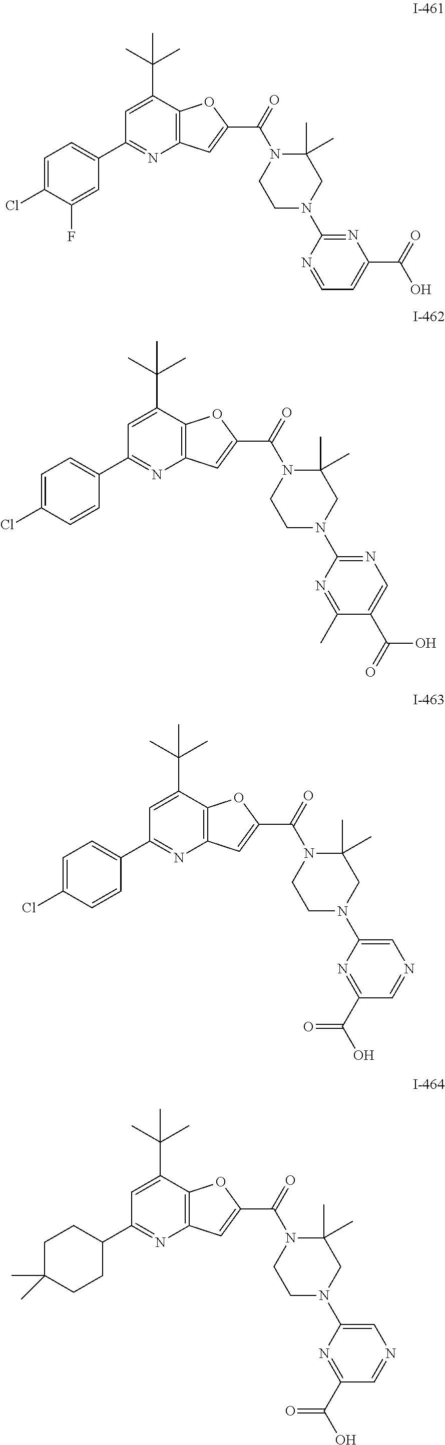 [SMILES: CC(C)(C)c1cc(-c2ccc(Cl)c(F)c2)nc2cc(C(=O)N3CCN(c4nccc(C(=O)O)n4)CC3(C)C)oc12 ; Cc1nc(N2CCN(C(=O)c3cc4nc(-c5ccc(Cl)cc5)cc(C(C)(C)C)c4o3)C(C)(C)C2)ncc1C(=O)O ; CC(C)(C)c1cc(-c2ccc(Cl)cc2)nc2cc(C(=O)N3CCN(c4cncc(C(=O)O)n4)CC3(C)C)oc12 ; CC1(C)CCC(c2cc(C(C)(C)C)c3oc(C(=O)N4CCN(c5cncc(C(=O)O)n5)CC4(C)C)cc3n2)CC1]

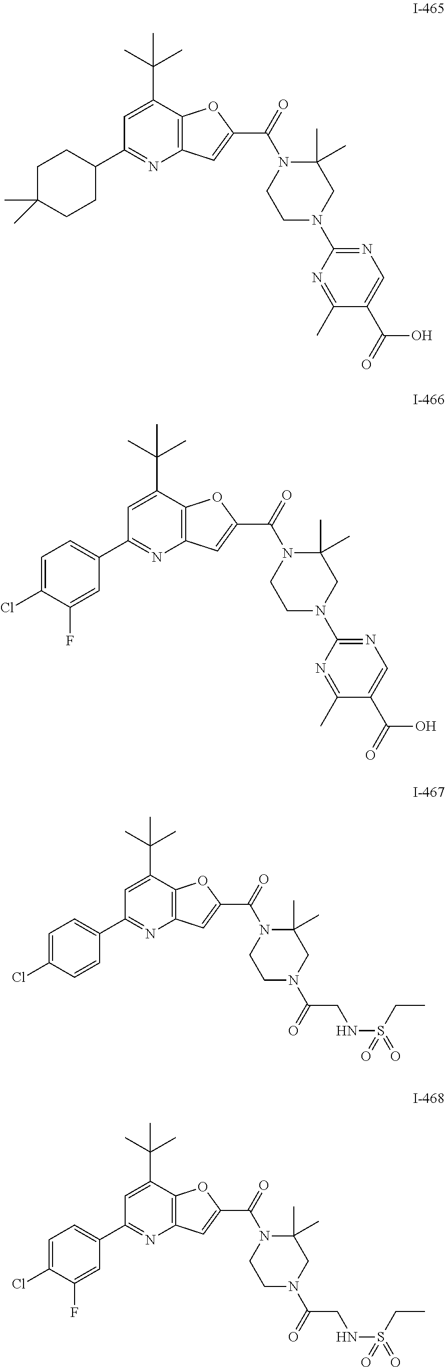 [SMILES: Cc1nc(N2CCN(C(=O)c3cc4nc(C5CCC(C)(C)CC5)cc(C(C)(C)C)c4o3)C(C)(C)C2)ncc1C(=O)O ; Cc1nc(N2CCN(C(=O)c3cc4nc(-c5ccc(Cl)c(F)c5)cc(C(C)(C)C)c4o3)C(C)(C)C2)ncc1C(=O)O ; CCS(=O)(=O)NCC(=O)N1CCN(C(=O)c2cc3nc(-c4ccc(Cl)cc4)cc(C(C)(C)C)c3o2)C(C)(C)C1 ; CCS(=O)(=O)NCC(=O)N1CCN(C(=O)c2cc3nc(-c4ccc(Cl)c(F)c4)cc(C(C)(C)C)c3o2)C(C)(C)C1]

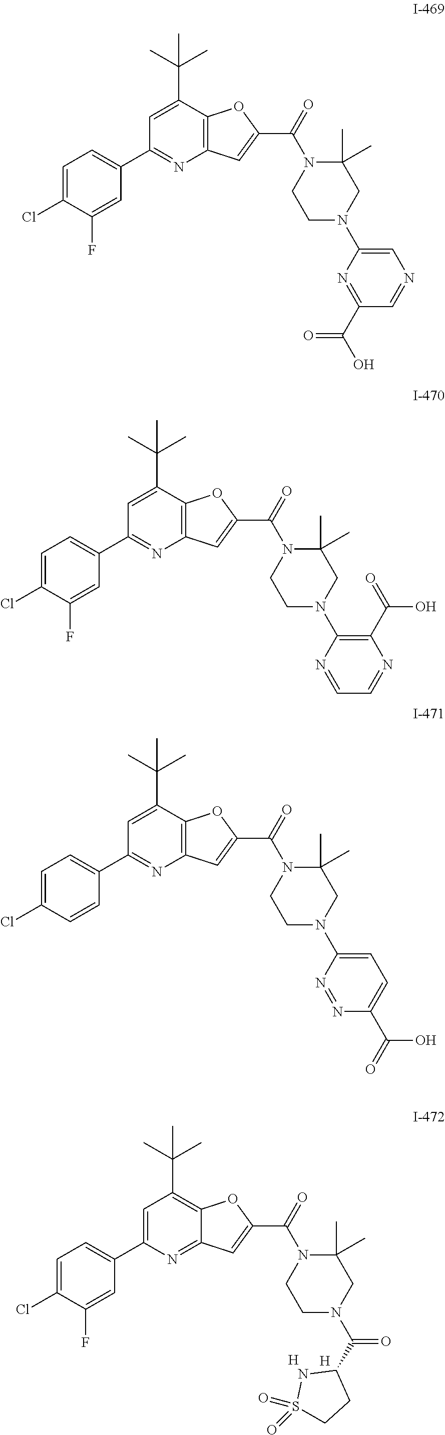

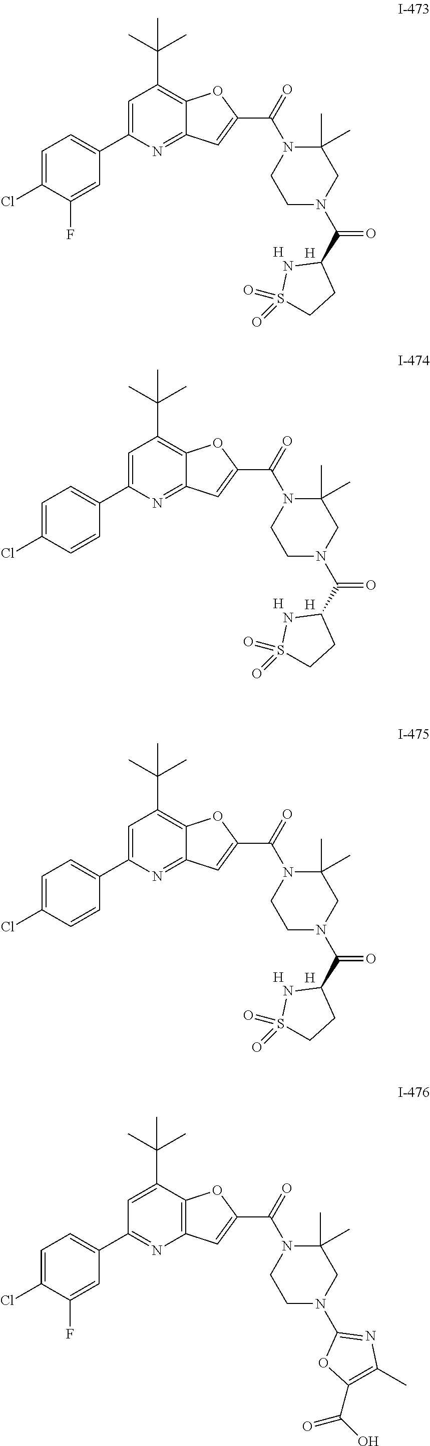

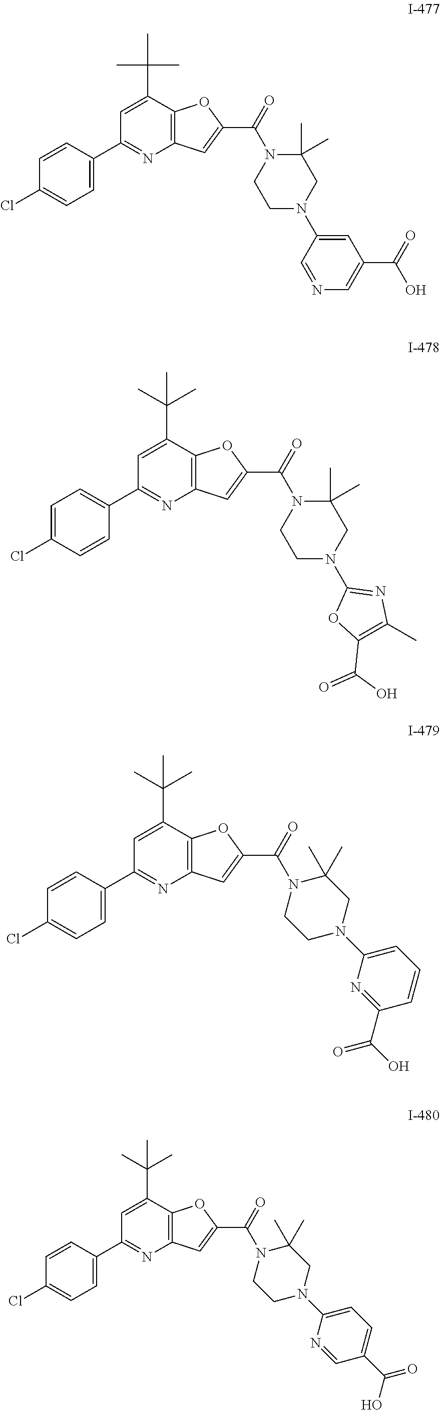 [SMILES: CC(C)(C)c1cc(-c2ccc(Cl)cc2)nc2cc(C(=O)N3CCN(c4cncc(C(=O)O)c4)CC3(C)C)oc12 ; Cc1nc(N2CCN(C(=O)c3cc4nc(-c5ccc(Cl)cc5)cc(C(C)(C)C)c4o3)C(C)(C)C2)oc1C(=O)O ; CC(C)(C)c1cc(-c2ccc(Cl)cc2)nc2cc(C(=O)N3CCN(c4cccc(C(=O)O)n4)CC3(C)C)oc12 ; CC(C)(C)c1cc(-c2ccc(Cl)cc2)nc2cc(C(=O)N3CCN(c4ccc(C(=O)O)cn4)CC3(C)C)oc12]

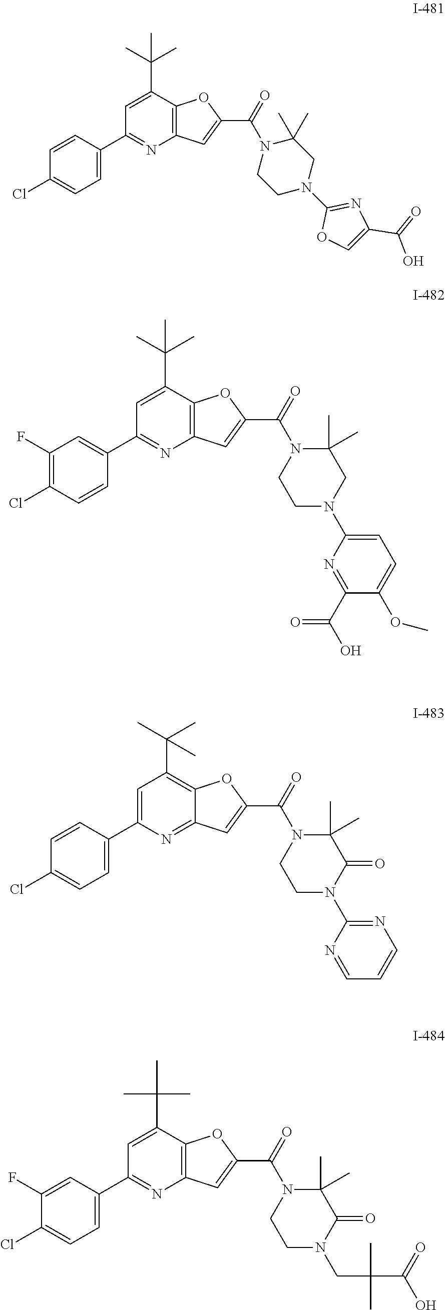 [SMILES: CC(C)(C)c1cc(-c2ccc(Cl)cc2)nc2cc(C(=O)N3CCN(c4nc(C(=O)O)co4)CC3(C)C)oc12 ; COc1ccc(N2CCN(C(=O)c3cc4nc(-c5ccc(Cl)c(F)c5)cc(C(C)(C)C)c4o3)C(C)(C)C2)nc1C(=O)O ; CC(C)(C)c1cc(-c2ccc(Cl)cc2)nc2cc(C(=O)N3CCN(c4ncccn4)C(=O)C3(C)C)oc12 ; CC(C)(CN1CCN(C(=O)c2cc3nc(-c4ccc(Cl)c(F)c4)cc(C(C)(C)C)c3o2)C(C)(C)C1=O)C(=O)O]

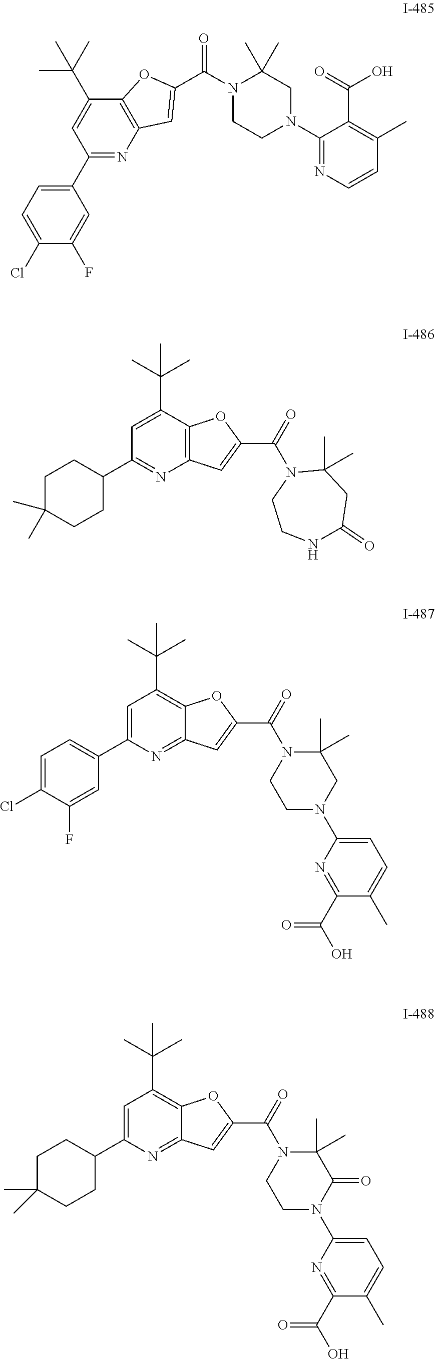

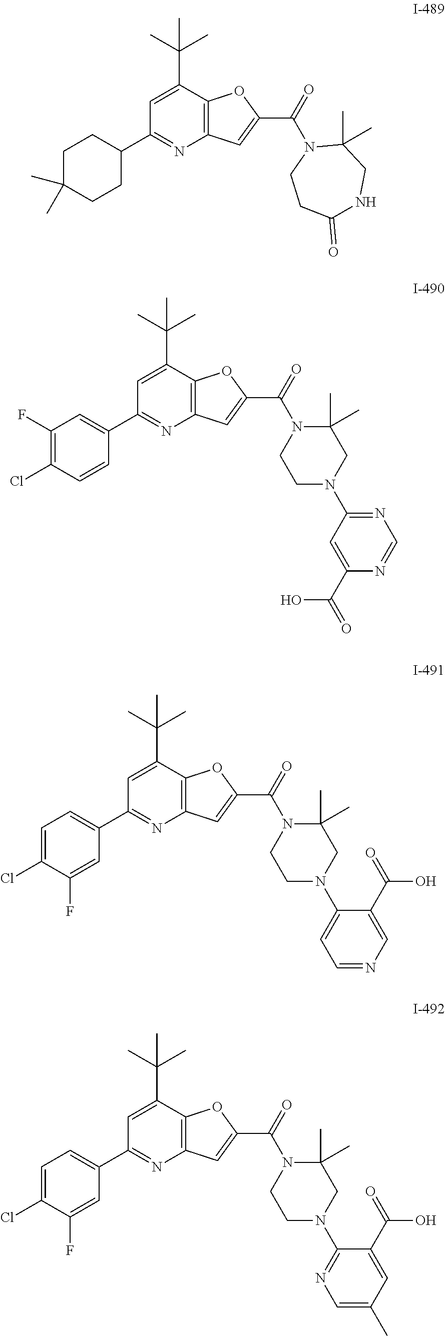

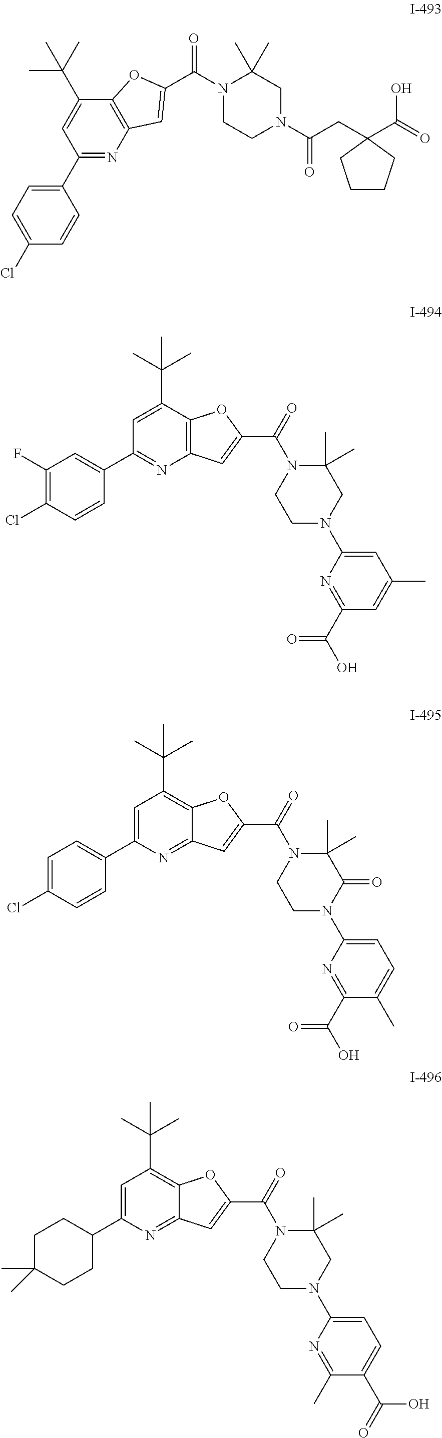

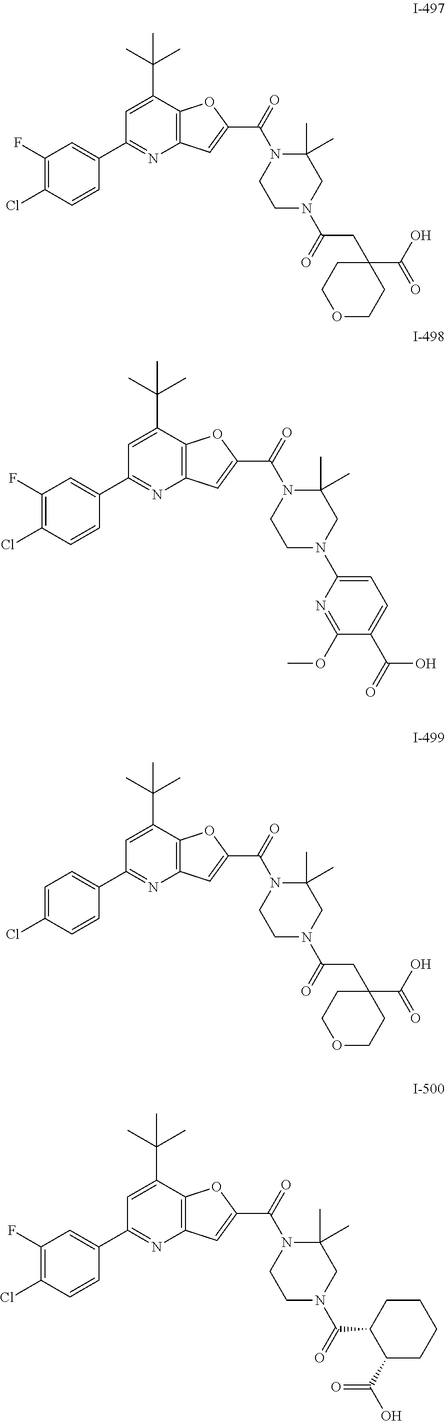 [SMILES: CC(C)(C)c1cc(-c2ccc(Cl)c(F)c2)nc2cc(C(=O)N3CCN(C(=O)CC4(C(=O)O)CCOCC4)CC3(C)C)oc12 ; COc1nc(N2CCN(C(=O)c3cc4nc(-c5ccc(Cl)c(F)c5)cc(C(C)(C)C)c4o3)C(C)(C)C2)ccc1C(=O)O ; CC(C)(C)c1cc(-c2ccc(Cl)cc2)nc2cc(C(=O)N3CCN(C(=O)CC4(C(=O)O)CCOCC4)CC3(C)C)oc12 ; CC(C)(C)c1cc(-c2ccc(Cl)c(F)c2)nc2cc(C(=O)N3CCN(C(=O)[C@@H]4CCCC[C@@H]4C(=O)O)CC3(C)C)oc12]

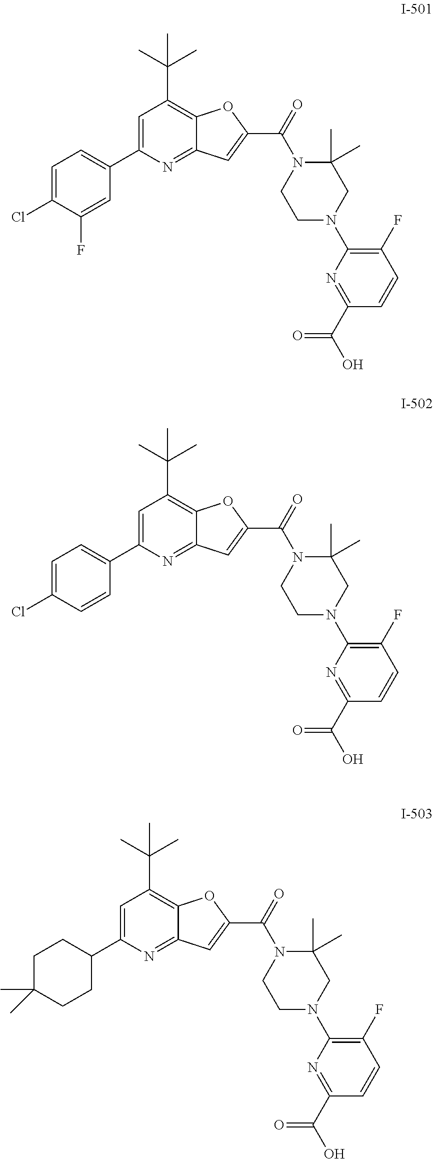 [SMILES: CC(C)(C)c1cc(-c2ccc(Cl)c(F)c2)nc2cc(C(=O)N3CCN(c4nc(C(=O)O)ccc4F)CC3(C)C)oc12 ; CC(C)(C)c1cc(-c2ccc(Cl)cc2)nc2cc(C(=O)N3CCN(c4nc(C(=O)O)ccc4F)CC3(C)C)oc12 ; CC1(C)CCC(c2cc(C(C)(C)C)c3oc(C(=O)N4CCN(c5nc(C(=O)O)ccc5F)CC4(C)C)cc3n2)CC1]

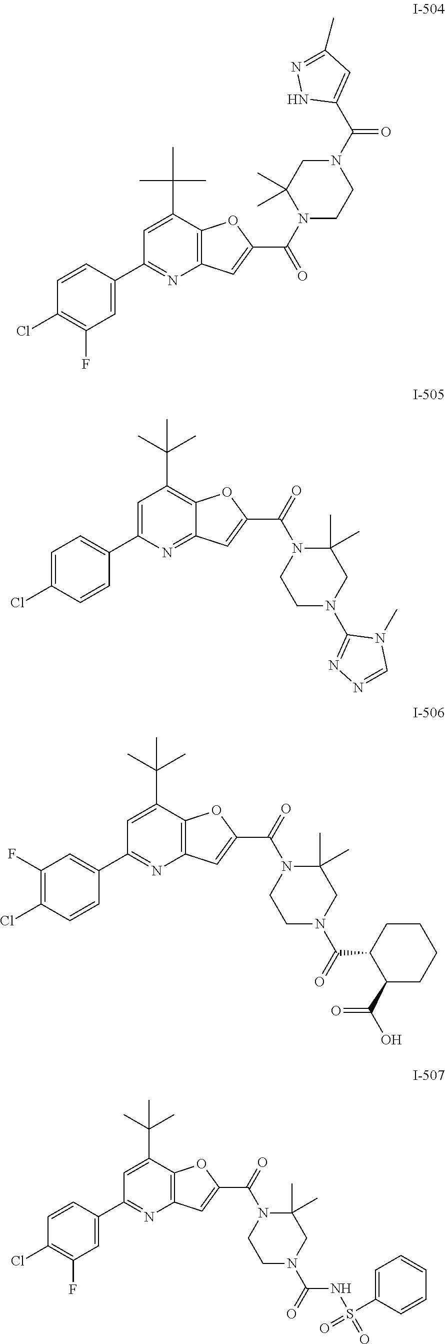 [SMILES: Cc1cc(C(=O)N2CCN(C(=O)c3cc4nc(-c5ccc(Cl)c(F)c5)cc(C(C)(C)C)c4o3)C(C)(C)C2)[nH]n1 ; Cn1cnnc1N1CCN(C(=O)c2cc3nc(-c4ccc(Cl)cc4)cc(C(C)(C)C)c3o2)C(C)(C)C1 ; CC(C)(C)c1cc(-c2ccc(Cl)c(F)c2)nc2cc(C(=O)N3CCN(C(=O)[C@@H]4CCCC[C@H]4C(=O)O)CC3(C)C)oc12 ; CC(C)(C)c1cc(-c2ccc(Cl)c(F)c2)nc2cc(C(=O)N3CCN(C(=O)NS(=O)(=O)c4ccccc4)CC3(C)C)oc12]

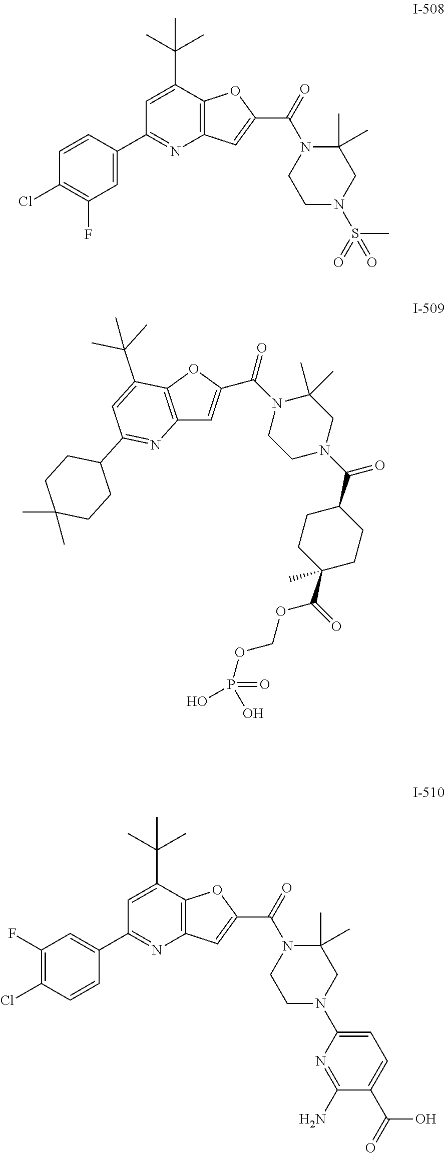

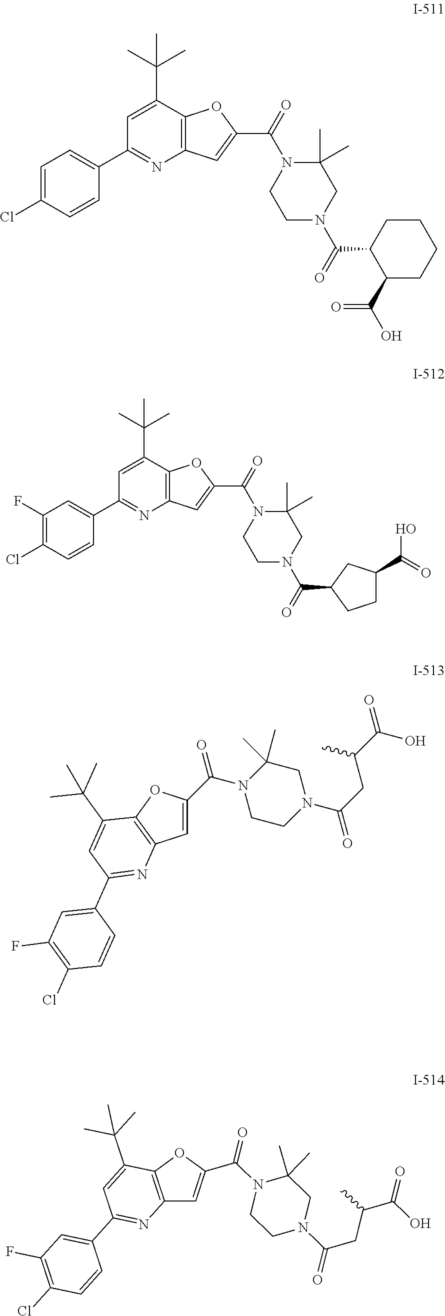

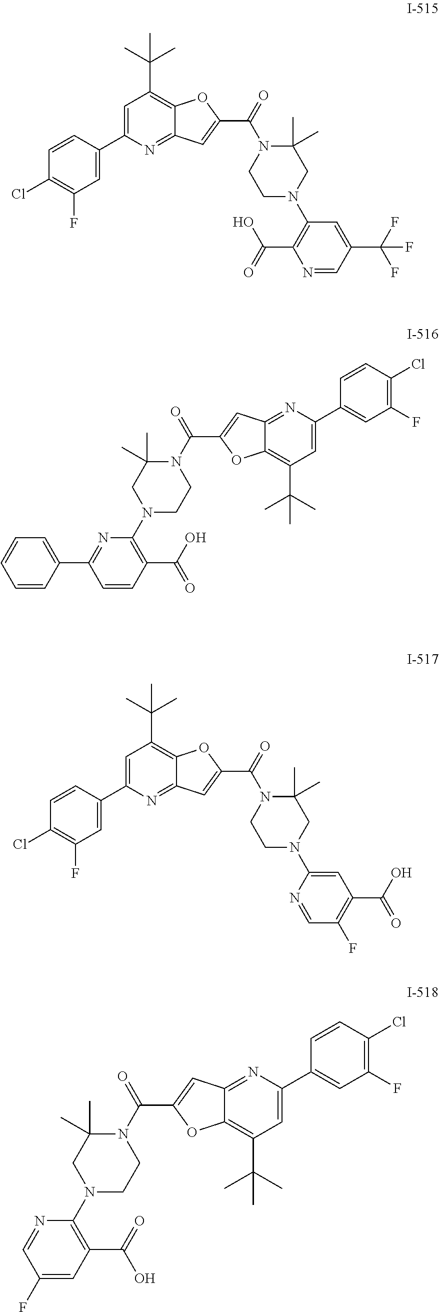 [SMILES: CC(C)(C)c1cc(-c2ccc(Cl)c(F)c2)nc2cc(C(=O)N3CCN(c4cc(C(F)(F)F)cnc4C(=O)O)CC3(C)C)oc12 ; CC(C)(C)c1cc(-c2ccc(Cl)c(F)c2)nc2cc(C(=O)N3CCN(c4nc(-c5ccccc5)ccc4C(=O)O)CC3(C)C)oc12 ; CC(C)(C)c1cc(-c2ccc(Cl)c(F)c2)nc2cc(C(=O)N3CCN(c4cc(C(=O)O)c(F)cn4)CC3(C)C)oc12 ; CC(C)(C)c1cc(-c2ccc(Cl)c(F)c2)nc2cc(C(=O)N3CCN(c4ncc(F)cc4C(=O)O)CC3(C)C)oc12]

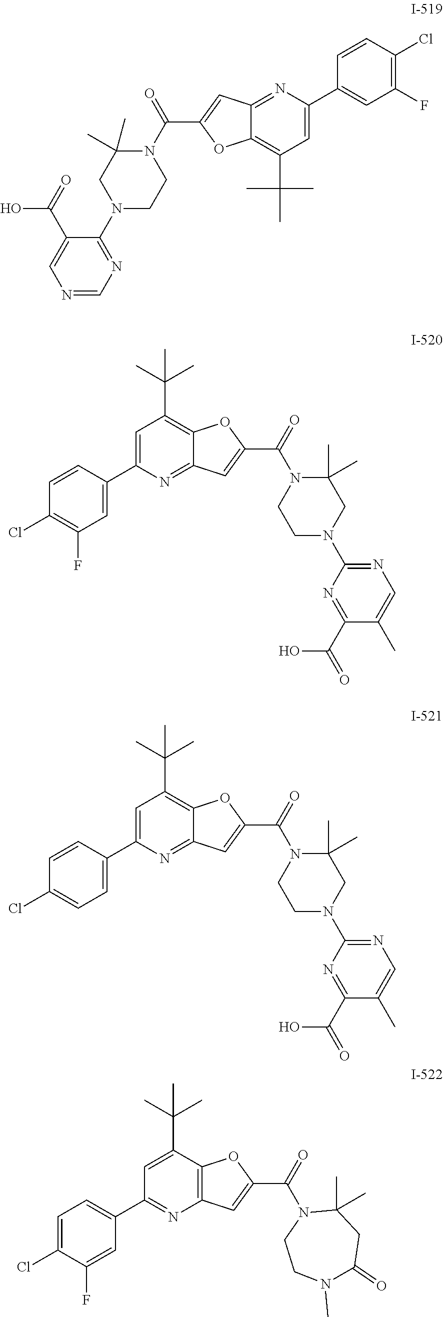 [SMILES: CC(C)(C)c1cc(-c2ccc(Cl)c(F)c2)nc2cc(C(=O)N3CCN(c4ncncc4C(=O)O)CC3(C)C)oc12 ; Cc1cnc(N2CCN(C(=O)c3cc4nc(-c5ccc(Cl)c(F)c5)cc(C(C)(C)C)c4o3)C(C)(C)C2)nc1C(=O)O ; Cc1cnc(N2CCN(C(=O)c3cc4nc(-c5ccc(Cl)cc5)cc(C(C)(C)C)c4o3)C(C)(C)C2)nc1C(=O)O ; CN1CCN(C(=O)c2cc3nc(-c4ccc(Cl)c(F)c4)cc(C(C)(C)C)c3o2)C(C)(C)CC1=O]

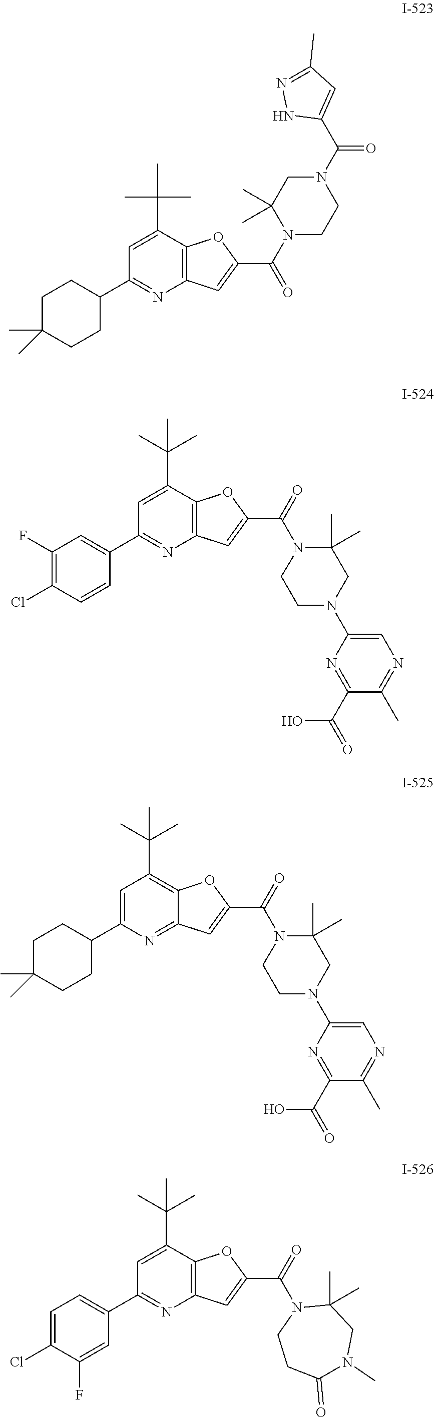

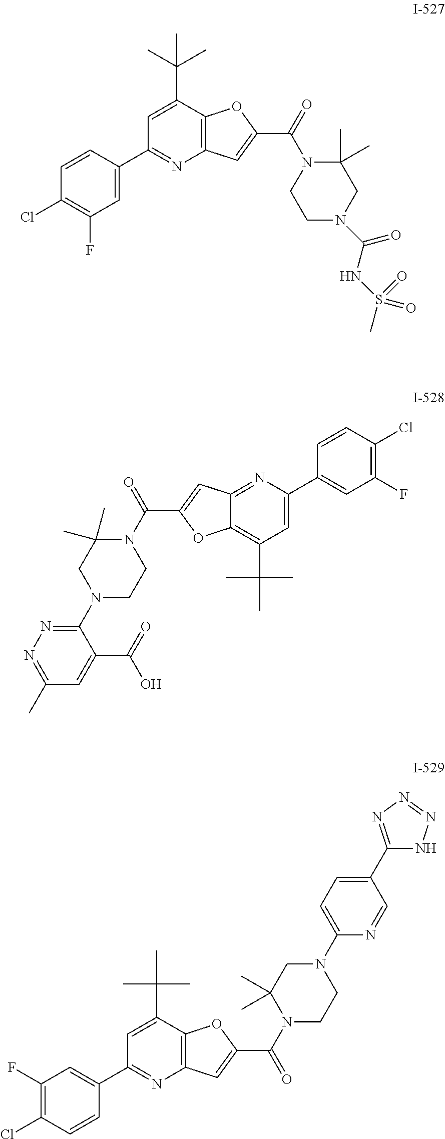 [SMILES: CC(C)(C)c1cc(-c2ccc(Cl)c(F)c2)nc2cc(C(=O)N3CCN(C(=O)NS(C)(=O)=O)CC3(C)C)oc12 ; Cc1cc(C(=O)O)c(N2CCN(C(=O)c3cc4nc(-c5ccc(Cl)c(F)c5)cc(C(C)(C)C)c4o3)C(C)(C)C2)nn1 ; CC(C)(C)c1cc(-c2ccc(Cl)c(F)c2)nc2cc(C(=O)N3CCN(c4ccc(-c5nnn[nH]5)cn4)CC3(C)C)oc12]

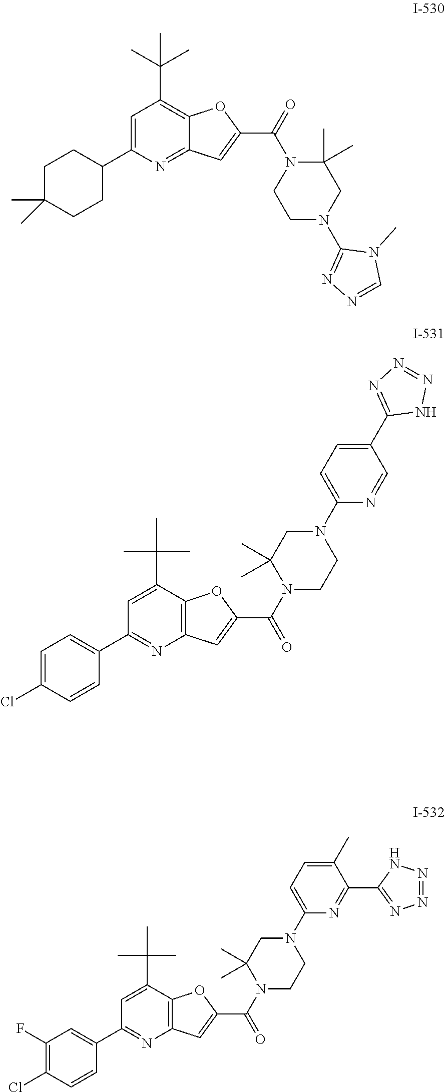

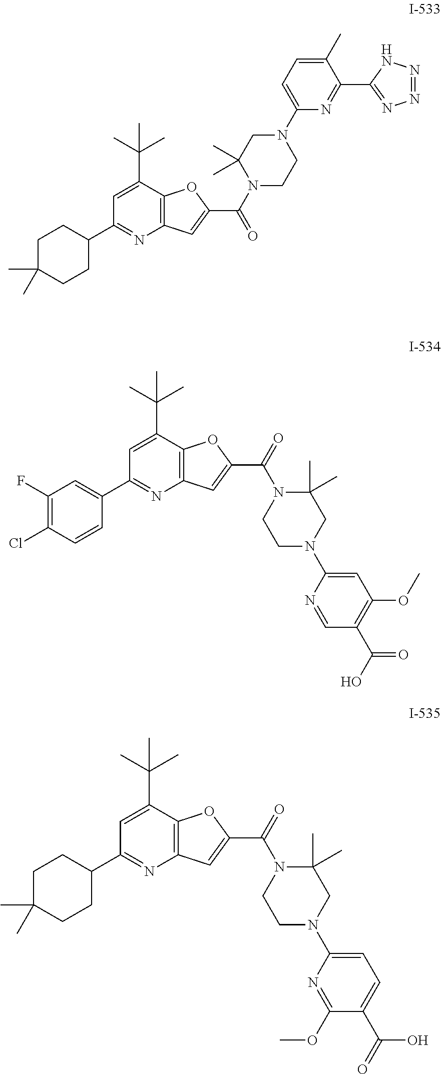

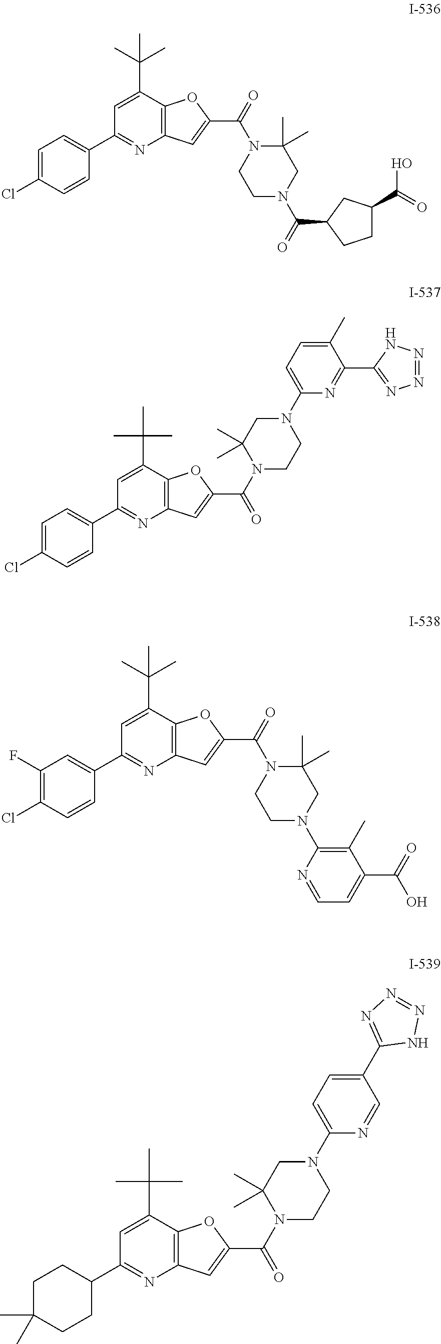 [SMILES: CC(C)(C)c1cc(-c2ccc(Cl)cc2)nc2cc(C(=O)N3CCN(C(=O)[C@@H]4CC[C@H](C(=O)O)C4)CC3(C)C)oc12 ; Cc1ccc(N2CCN(C(=O)c3cc4nc(-c5ccc(Cl)cc5)cc(C(C)(C)C)c4o3)C(C)(C)C2)nc1-c1nnn[nH]1 ; Cc1c(C(=O)O)ccnc1N1CCN(C(=O)c2cc3nc(-c4ccc(Cl)c(F)c4)cc(C(C)(C)C)c3o2)C(C)(C)C1 ; CC1(C)CCC(c2cc(C(C)(C)C)c3oc(C(=O)N4CCN(c5ccc(-c6nnn[nH]6)cn5)CC4(C)C)cc3n2)CC1]

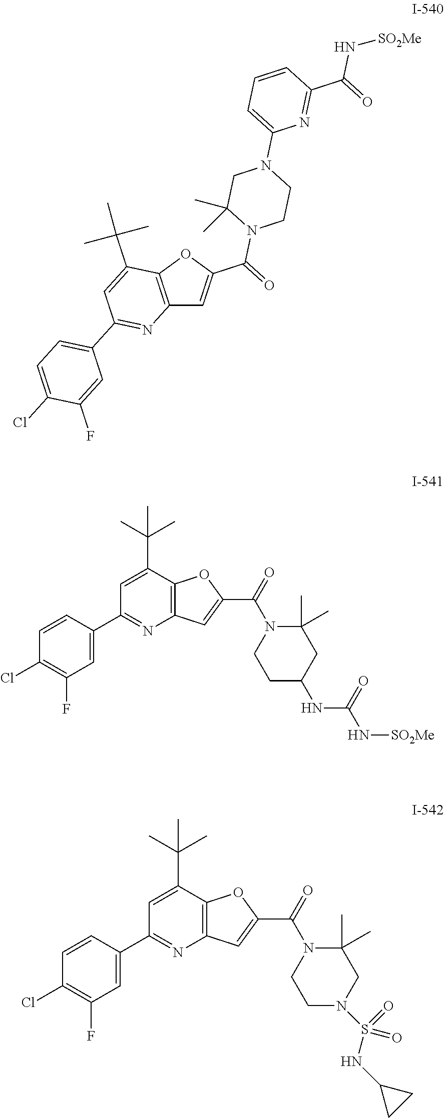 [SMILES: CC(C)(C)c1cc(-c2ccc(Cl)c(F)c2)nc2cc(C(=O)N3CCN(c4cccc(C(=O)NS(C)(=O)=O)n4)CC3(C)C)oc12 ; CC(C)(C)c1cc(-c2ccc(Cl)c(F)c2)nc2cc(C(=O)N3CCC(NC(=O)NS(C)(=O)=O)CC3(C)C)oc12 ; CC(C)(C)c1cc(-c2ccc(Cl)c(F)c2)nc2cc(C(=O)N3CCN(S(=O)(=O)NC4CC4)CC3(C)C)oc12]

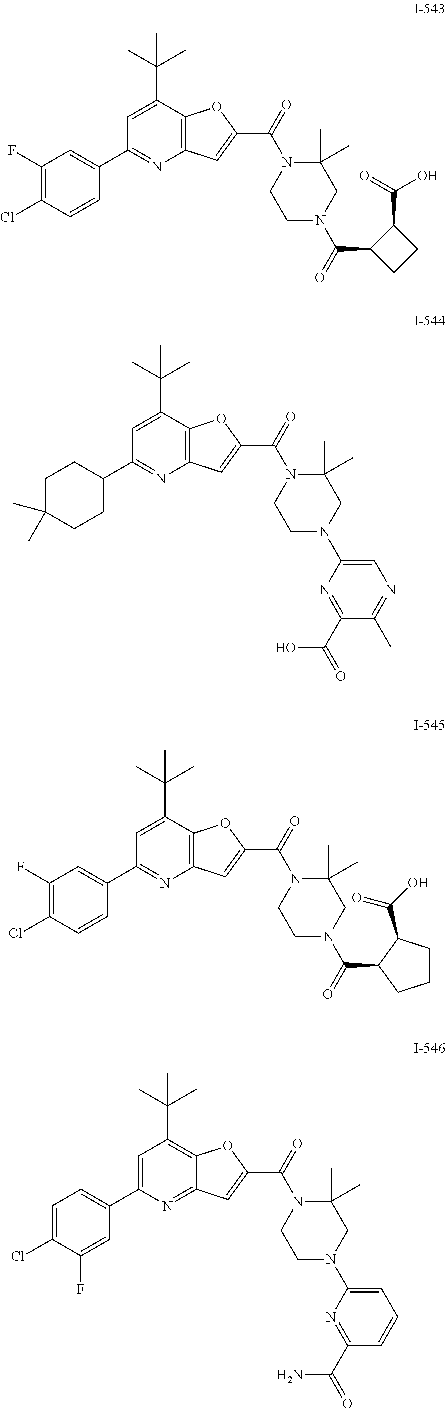

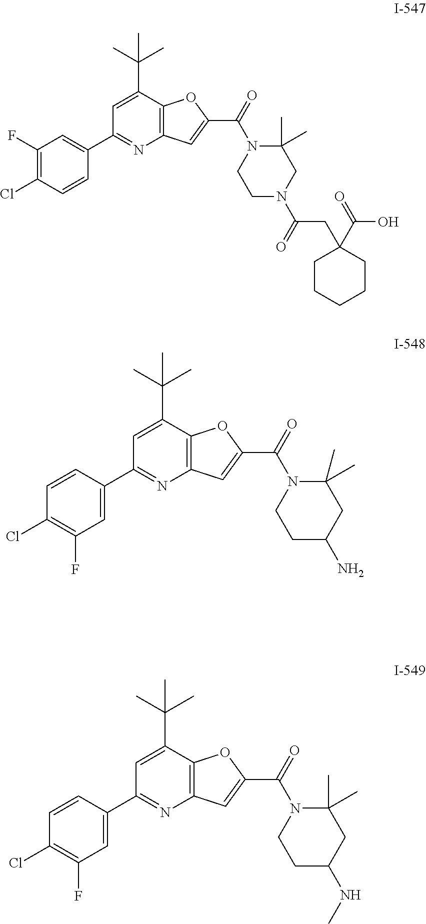 [SMILES: CC(C)(C)c1cc(-c2ccc(Cl)c(F)c2)nc2cc(C(=O)N3CCN(C(=O)CC4(C(=O)O)CCCCC4)CC3(C)C)oc12 ; CC(C)(C)c1cc(-c2ccc(Cl)c(F)c2)nc2cc(C(=O)N3CCC(N)CC3(C)C)oc12 ; CNC1CCN(C(=O)c2cc3nc(-c4ccc(Cl)c(F)c4)cc(C(C)(C)C)c3o2)C(C)(C)C1]

54. The compound of claim 53, represented by any one of the following chemical structures or a pharmaceutically acceptable salt thereof:

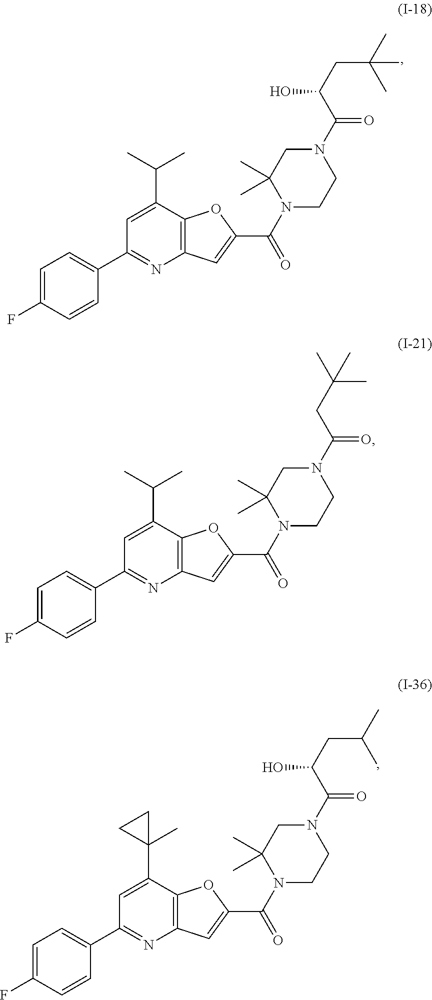

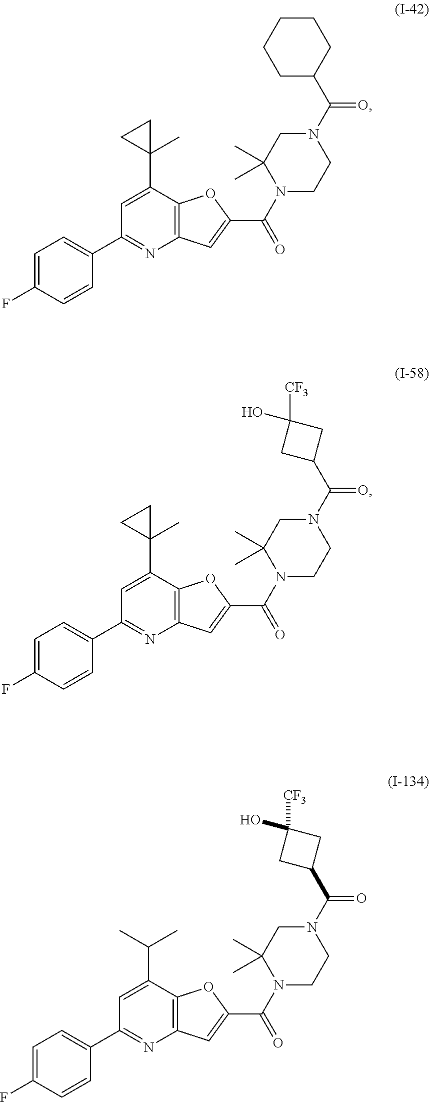

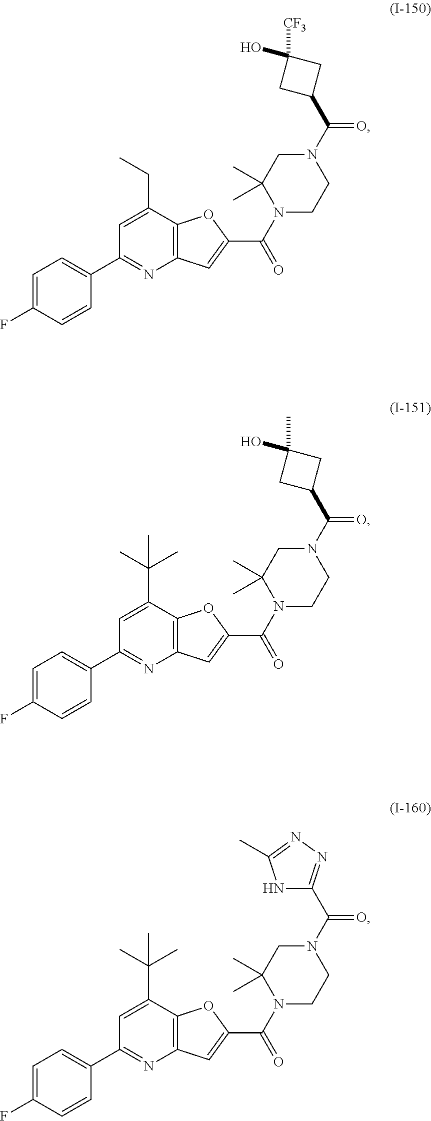

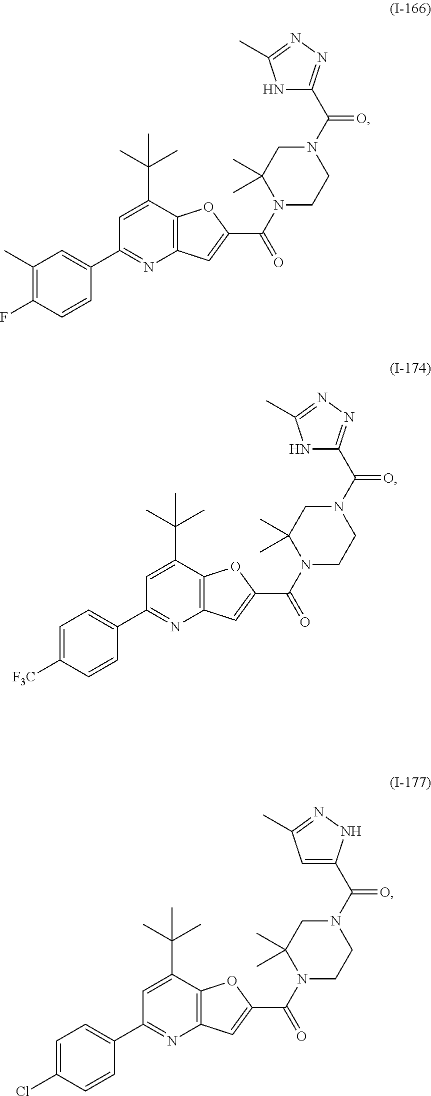 [SMILES: Cc1nnc(C(=O)N2CCN(C(=O)c3cc4nc(-c5ccc(F)c(C)c5)cc(C(C)(C)C)c4o3)C(C)(C)C2)[nH]1 ; Cc1nnc(C(=O)N2CCN(C(=O)c3cc4nc(-c5ccc(C(F)(F)F)cc5)cc(C(C)(C)C)c4o3)C(C)(C)C2)[nH]1 ; Cc1cc(C(=O)N2CCN(C(=O)c3cc4nc(-c5ccc(Cl)cc5)cc(C(C)(C)C)c4o3)C(C)(C)C2)[nH]n1]

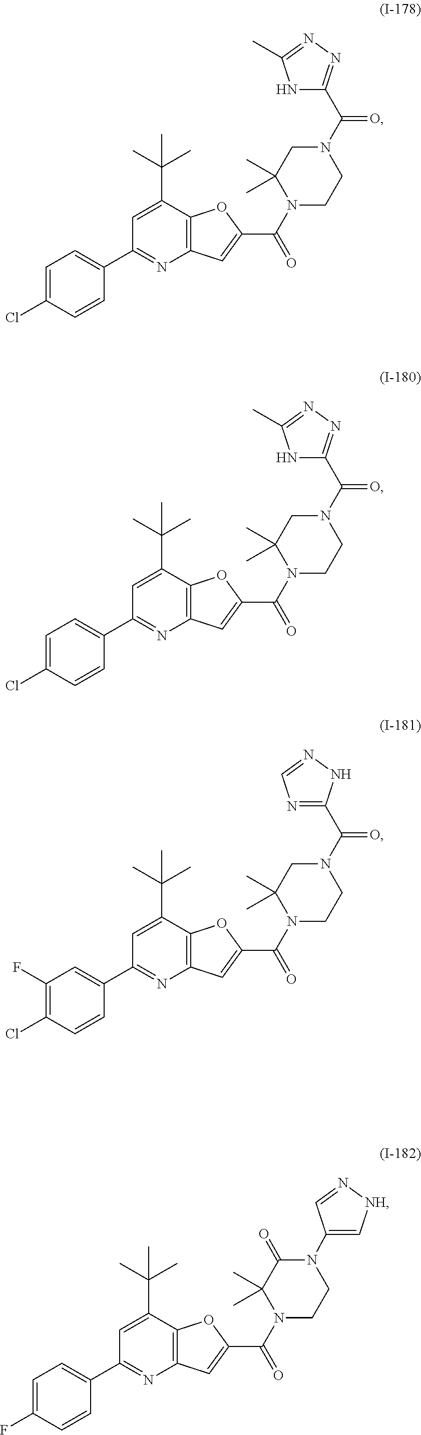 [SMILES: Cc1nnc(C(=O)N2CCN(C(=O)c3cc4nc(-c5ccc(Cl)cc5)cc(C(C)(C)C)c4o3)C(C)(C)C2)[nH]1 ; Cc1nnc(C(=O)N2CCN(C(=O)c3cc4nc(-c5ccc(Cl)cc5)cc(C(C)(C)C)c4o3)C(C)(C)C2)[nH]1 ; CC(C)(C)c1cc(-c2ccc(Cl)c(F)c2)nc2cc(C(=O)N3CCN(C(=O)c4ncn[nH]4)CC3(C)C)oc12 ; CC(C)(C)c1cc(-c2ccc(F)cc2)nc2cc(C(=O)N3CCN(c4cn[nH]c4)C(=O)C3(C)C)oc12]

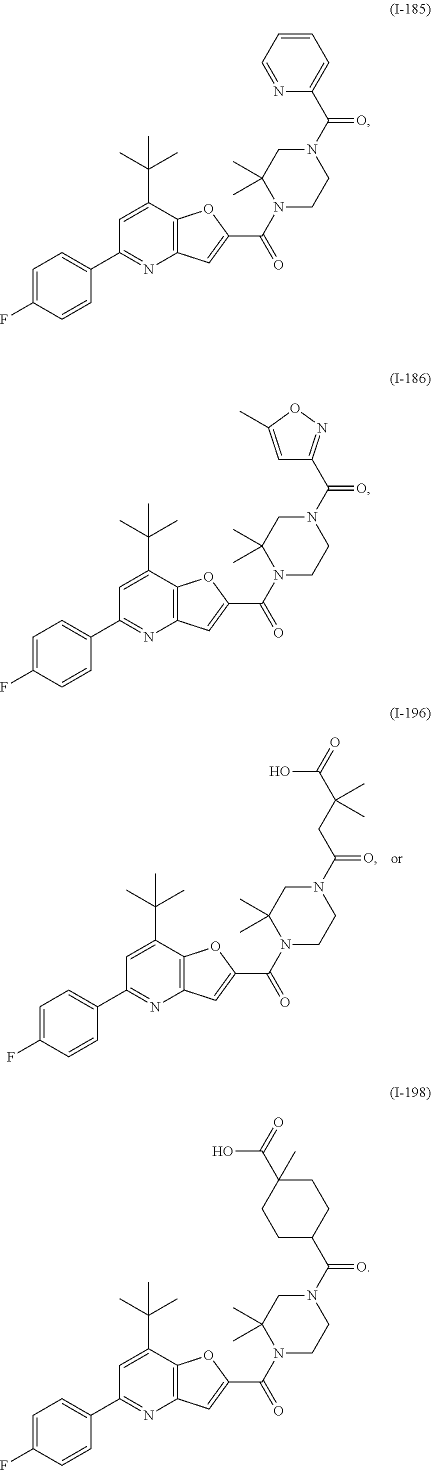 [SMILES: CC(C)(C)c1cc(-c2ccc(F)cc2)nc2cc(C(=O)N3CCN(C(=O)c4ccccn4)CC3(C)C)oc12 ; Cc1cc(C(=O)N2CCN(C(=O)c3cc4nc(-c5ccc(F)cc5)cc(C(C)(C)C)c4o3)C(C)(C)C2)no1 ; CC(C)(CC(=O)N1CCN(C(=O)c2cc3nc(-c4ccc(F)cc4)cc(C(C)(C)C)c3o2)C(C)(C)C1)C(=O)O ; CC1(C(=O)O)CCC(C(=O)N2CCN(C(=O)c3cc4nc(-c5ccc(F)cc5)cc(C(C)(C)C)c4o3)C(C)(C)C2)CC1]

55. The compound of claim 53, represented by any one of the following chemical structures or a pharmaceutically acceptable salt thereof:

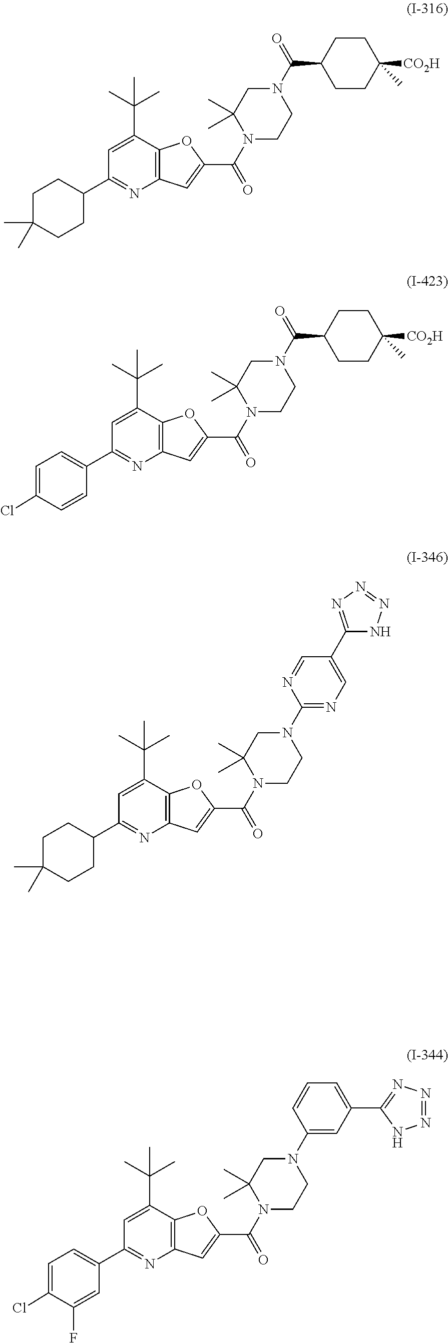 [SMILES: CC1(C)CCC(c2cc(C(C)(C)C)c3oc(C(=O)N4CCN(C(=O)[C@H]5CC[C@@](C)(C(=O)O)CC5)CC4(C)C)cc3n2)CC1 ; CC(C)(C)c1cc(-c2ccc(Cl)cc2)nc2cc(C(=O)N3CCN(C(=O)[C@H]4CC[C@@](C)(C(=O)O)CC4)CC3(C)C)oc12 ; CC1(C)CCC(c2cc(C(C)(C)C)c3oc(C(=O)N4CCN(c5ncc(-c6nnn[nH]6)cn5)CC4(C)C)cc3n2)CC1 ; CC(C)(C)c1cc(-c2ccc(Cl)c(F)c2)nc2cc(C(=O)N3CCN(c4cccc(-c5nnn[nH]5)c4)CC3(C)C)oc12]

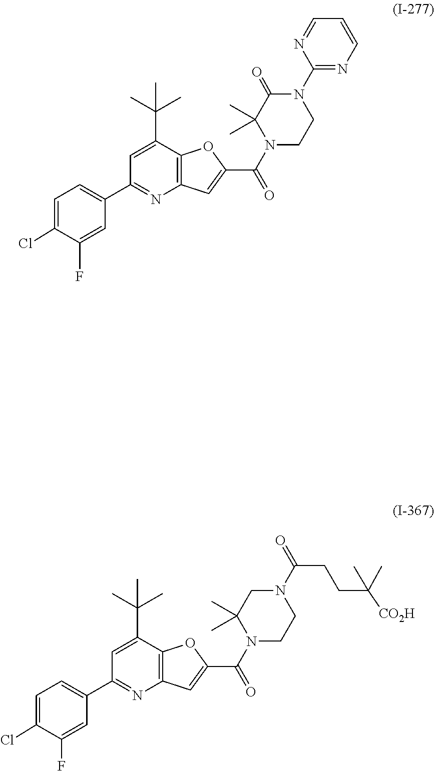 [SMILES: CC(C)(C)c1cc(-c2ccc(Cl)c(F)c2)nc2cc(C(=O)N3CCN(c4ncccn4)C(=O)C3(C)C)oc12 ; CC(C)(CCC(=O)N1CCN(C(=O)c2cc3nc(-c4ccc(Cl)c(F)c4)cc(C(C)(C)C)c3o2)C(C)(C)C1)C(=O)O]

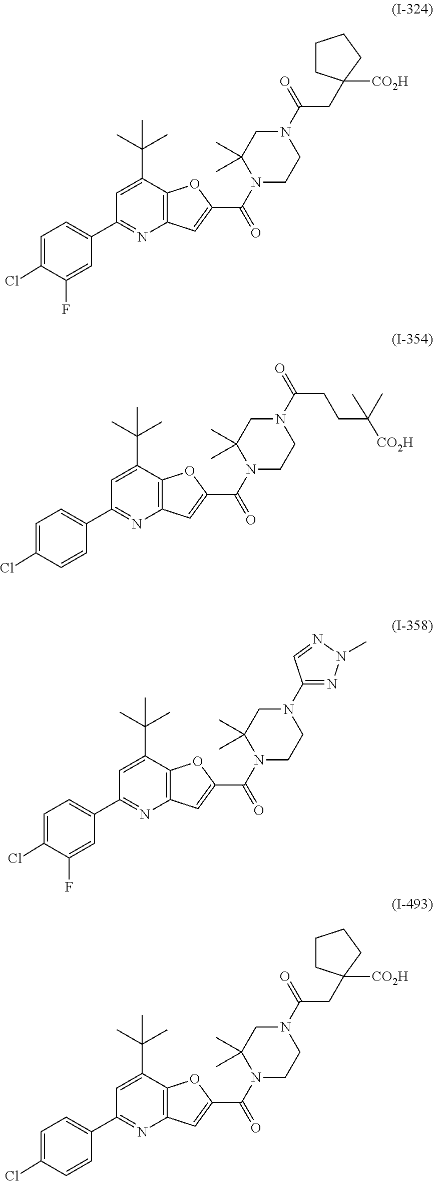 [SMILES: CC(C)(C)c1cc(-c2ccc(Cl)c(F)c2)nc2cc(C(=O)N3CCN(C(=O)CC4(C(=O)O)CCCC4)CC3(C)C)oc12 ; CC(C)(CCC(=O)N1CCN(C(=O)c2cc3nc(-c4ccc(Cl)cc4)cc(C(C)(C)C)c3o2)C(C)(C)C1)C(=O)O ; Cn1ncc(N2CCN(C(=O)c3cc4nc(-c5ccc(Cl)c(F)c5)cc(C(C)(C)C)c4o3)C(C)(C)C2)n1 ; CC(C)(C)c1cc(-c2ccc(Cl)cc2)nc2cc(C(=O)N3CCN(C(=O)CC4(C(=O)O)CCCC4)CC3(C)C)oc12]

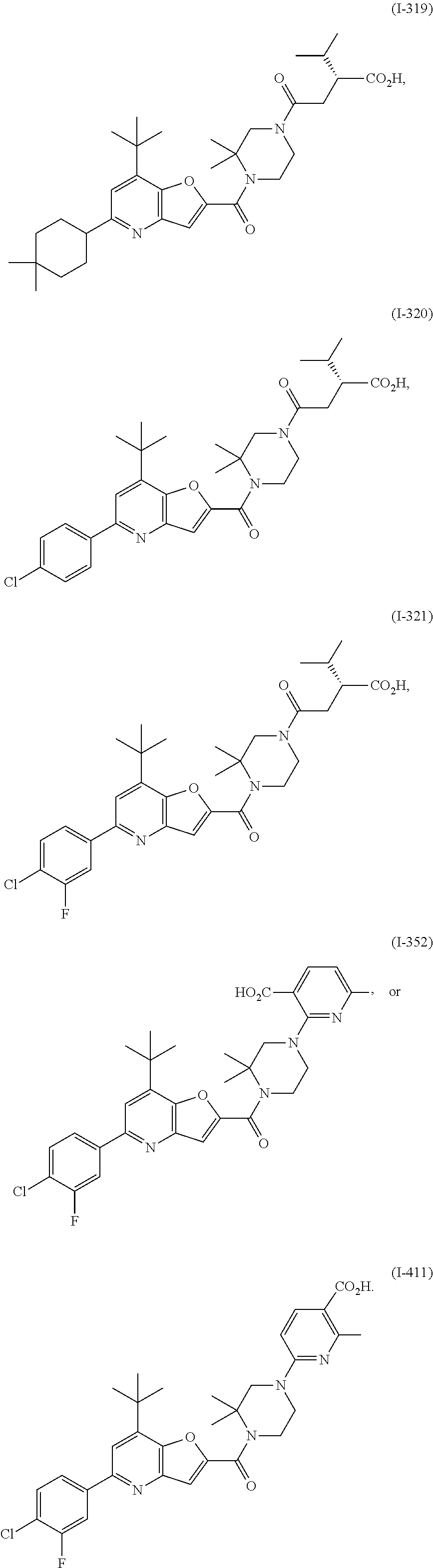

56. A pharmaceutical composition comprising a compound of Formula (I) or a pharmaceutically acceptable salt thereof: 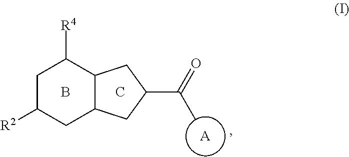
wherein the variables of Formula (I) are each and independently as described in any one of claims 1-55; and a pharmaceutically acceptable carrier, adjuvant, or excipient.

57. A method for treating a PAR-2 mediated disease in a patient comprising administering to the patient a compound of any one of claims 1-55 or a pharmaceutical composition of claim 56.

58. A method for treating, preventing, or reducing inflammation or nociception (pain) in a patient comprising administering to the patient a compound of any one of claims 1-55 or a pharmaceutical composition of claim 56.

59. A method for treating inflammatory bowel disease, Crohn's disease, irritable bowel syndrome, ulcerative colitis, asthma, rheumatoid arthritis, osteoarthritis, gingivitis, atopic dermatitis, psoriasis, systemic lupus erythematosus (SLE), scleroderma, interstitial lung disease, polymyositis, periodontitis, vasculitis, Netherton syndrome, atopic dermatitis, dermatomyositis, uveitis, Alzheimer's disease, Parkinson's disease, multiple sclerosis, inflammatory pain, post-operative incision pain, neuropathic pain, fracture pain, osteroporotic fracture pain, gout joint pain, fibrosis, cancer, diet-induced obesity, adipose inflammation, and metabolic dysfunction correlating with PAR2 expression in a patient comprising administering a compound of any one of claims 1-55 or a pharmaceutical composition of claim 56.

60. The method of claim 59, wherein the fibrosis is liver fibrosis, pulmonary fibrosis, cystic fibrosis, renal fibrosis, peritoneal fibrosis, pancreatic fibrosis, or cardiac fibrosis.

61. The method of claim 59, wherein the cancer is a protease-driven cancer.

62. The method of claim 59, wherein the method is for treating atopic dermatitis.

63. A method of inhibiting proteolytic activation of PAR-2 in a cell comprising administering to a patient or to a biological sample a compound of any one of claims 1-55 or a pharmaceutical composition of claim 56.

64. A method of inhibiting PAR-2 activity in a cell comprising administering to a patient or to a biological sample a compound of any one of claims 1-55 or a pharmaceutical composition of claim 56.

65. A method of preparing a compound represented by Structural Formula (I) or a pharmaceutically acceptable salt thereof: 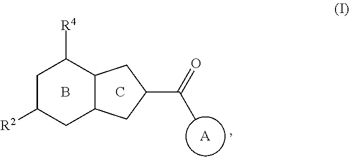
wherein Rings A, B, and C, and R² and R⁴ are each and independently as defined in any one of claims 1-55, comprising:
reacting Compound (X-1) with Compound (Y-1) to form a compound of formula (I) or a pharmaceutically acceptable salt thereof: 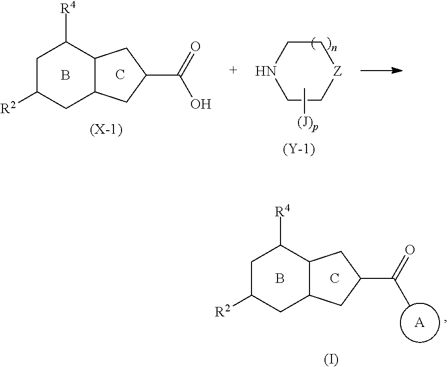
wherein the variables of Compound (X-1) with Compound (Y-1) are each and independently as described for formula (I) in any one of claims 1-55.

66. A method of preparing a compound represented by Structural Formula (I) or pharmaceutically acceptable salt thereof: 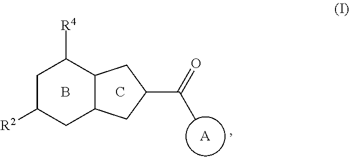
wherein Rings B and C, and R² and R⁴ are each and independently as defined in any one of claims 1-55, and Ring A is 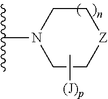
wherein Z is —NX—; comprising:
reacting Compound (X-2) with X-L¹ to form a compound of formula (I) or a pharmaceutically acceptable salt thereof: 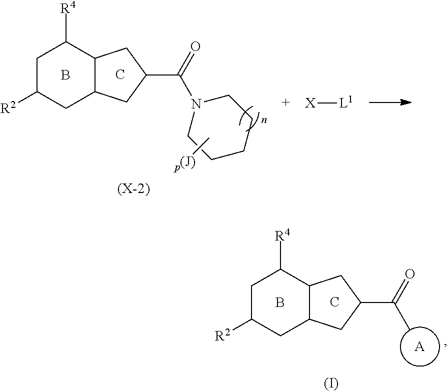
wherein X of X-L¹ is as described for formula (I) in any one of claims 1-53 and L¹ of X-L¹ is from halogen or —OH, and wherein the variables of Compound (X-2) are each and independently as described for Formula (I) in any one of claims 1-55.

67. A method of preparing a compound of formula (I) or pharmaceutically acceptable salt thereof: 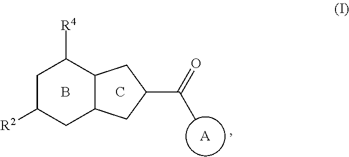
wherein the variables of formula (I) are each and independently as described above in any one of claims 1-55, comprising:
reacting Compound (X-3) with R²-L³ to form a compound of formula (I) or a pharmaceutically acceptable salt thereof: 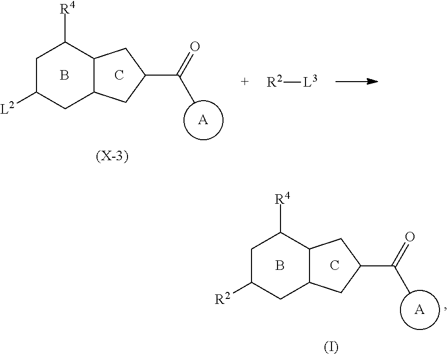
wherein L^{Z} of Compound (X-3) is halogen, and the remaining variables of Compound (X-3) are each and independently as described for formula (I) in any one of claims 1-55, and wherein L³ of R²-L³ is —B(OR^{a})₂, wherein R^{a} is —H or two R^{a} together with the atom to which they are attached form a dioxaborolane optionally substituted with C₁₋₂alkyl, and R² of R²-L³ is as described for formula (I) in any one of claims 1-55.
